(19) United States
(12) Patent Application Publication
(10) Pub. No.: US 2024/0343704 A1
(43) Pub. Date: Oct. 17, 2024

(54) HALO-SUBSTITUTED AMINO PYRIDINE COMPOUNDS AS INHIBITORS OF THE HAEMATOPOIETIC PROGENITOR KINASE 1 (HPK1)

(71) Applicant: Ontario Institute for Cancer Research (OICR), Toronto (CA)

(72) Inventors: Anh Chau, Scarborough (CA); Methvin Isaac, Brampton (CA); Babu Joseph, Oakville (CA); Radek Laufer, Oakville (CA); Gennady Poda, Toronto (CA); Michael Prakesch, Toronto (CA); Pandiaraju Subramanian, Oakville (CA); David Uehling, Toronto (CA); Iain Watson, Toronto (CA); Brian Wilson, Mississauga (CA); Tao Xin, Woodbridge (CA)

(73) Assignee: Ontario Institute for Cancer Research (OICR), Toronto, ON (CA)

(21) Appl. No.: 18/288,213

(22) PCT Filed: May 2, 2022
(86) PCT No.: PCT/CA2022/050678

§ 371 Date: Oct. 25, 2023

Related U.S. Application Data
(60) Provisional application No. 63/182,203, filed on Apr. 30, 2021.

(51) Int. Cl. C07D 401/04; A61K 31/4725; A61K 31/496; A61K 31/4995; A61K 31/517; A61K 31/519; A61K 31/53; A61K 31/5377; A61K 31/538; A61K 31/5383; A61K 31/5386; A61K 31/541; A61K 31/5415; A61K 31/55; A61K 31/551; A61K 31/553; A61K 31/695; C07D 401/14; C07D 405/14; C07D 413/04; C07D 413/14; C07D 417/04; C07D 471/04; C07D 471/10; C07D 487/04; C07D 487/10; C07D 491/048; C07D 491/107; C07D 491/113; C07D 495/04; C07D 498/04; C07D 498/10; C07F 7/08

(52) U.S. Cl. CPC C07D 401/04; A61K 31/4725; A61K 31/496; A61K 31/4995; A61K 31/517; A61K 31/519; A61K 31/53; A61K 31/5377; A61K 31/538; A61K 31/5383; A61K 31/5386; A61K 31/541; A61K 31/5415; A61K 31/55; A61K 31/551; A61K 31/553; A61K 31/695; C07D 401/14; C07D 405/14; C07D 413/04; C07D 413/14; C07D 417/04; C07D 471/04; C07D 471/10; C07D 487/04; C07D 487/10; C07D 491/048; C07D 491/107; C07D 491/113; C07D 495/04; C07D 498/04; C07D 498/10; C07F 7/0836

(57) ABSTRACT

The present application relates to halo-substituted heterocyclic compounds of Formula (I): or pharmaceutically acceptable salts, solvates and/or prodrugs thereof, to compositions comprising these compounds or pharmaceutically acceptable salts, solvates and/or prodrugs thereof, and various uses in the treatment of diseases, disorders or conditions that are treatable by inhibiting HPK1, such as cancer.

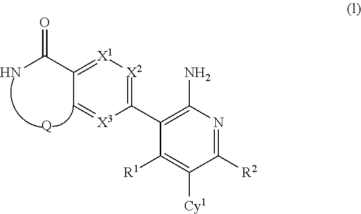

### RELATED APPLICATIONS

The present application claims the benefit of priority of co-pending U.S. provisional patent application No. 63/182,203 filed on Apr. 30, 2021, the contents of which are incorporated herein by reference in their entirety.

### FIELD

The present application relates to halo-substituted amino pyridine compounds, to processes for their preparation, to compositions comprising them, and to their use in therapy. More particularly, it relates to halo substituted amino pyridine compounds useful in the treatment of diseases, disorders or conditions treatable by inhibiting HPK1.

### BACKGROUND

Tumors are genetically heterogeneous and have evolved mechanisms to hijack cellular growth and regulatory pathways, which makes it unlikely a single therapy will have a significant impact on patient survival. For this reason, immunotherapy has become an important paradigm in the treatment of some types of cancers. Immune effector cells such as T-cells and B-cells can suppress the proliferation of cancer cells by targeting abnormal, tumor-expressed antigens. For example, recent clinical testing of novel immunotherapy strategies (e.g. anti-PD1 and anti-PDL1) has demonstrated unprecedented and durable survival benefit even in advanced patients suffering from metastatic cancers. However, the overall excitement for this therapeutic approach is tempered by the observation that these responses to agents targeting the PD-1 axis are limited to a minority of cancer patients. Hence, in order to broaden the response rates in cancer patients, there is an urgent need to build on the tremendous promise of immunotherapy to more rapidly test rational combinations of small molecules with immuno-therapeutics. One such approach is the combination of a small molecule hematopoietic progenitor kinase 1 (HPK1) inhibitor with current anti-PD1/PDL1 immunotherapies. An HPK1 inhibitor should potentiate anti-tumor immune responses by stimulating T-cell proliferation and triggering tumor cell senescence and tumor clearance by T cells.

The hematopoietic progenitor kinase 1 (HPK1, MAP4K1), is a T-cell receptor (TCR)-proximal kinase involved in the regulation of proliferation and survival of primary T cells [Nat Immunol. 2007; 8(1):84-91.] HPK1 is exclusively expressed in hematopoietic tissues and activates the c-Jun N-terminal kinase (JNK) and the NF-kB pathways [7]. Transient knockdown of HPK1 in T cells blocks activation of NF-kB [Crit Rev Oncol Hematol. 2008; 66(1):52-64]. Most strikingly, mice that received adoptive transfer of HPKI (−/−) T cells became resistant to lung tumor growth [Immunol Res. 2012; 54(1-3):262-5]. HPK1 has an N-terminal kinase domain and a C-terminal citron homology domain. Antigen receptor cross-linking leads to activation of HPK1 in T and B cells resulting in HPK1 relocation to the plasma membrane, autophosphorylation and transphosphorylation by protein kinase D1 (PKD1). Subsequent transphosphorylation by PKD1 and auto-phosphorylation within the kinase domain result in full activation of HPK1, which then regulates different cellular responses including apoptosis, activation-induced cell death and autoimmunity. HPK1 mediates negative regulation of the immune response via phosphorylation of SLP-76 (S376). Mutation of lysine-46 to methionine (designated HPK1-M46) in the ATP-binding site of the kinase domain abolishes catalytic activation of HPK1 resulting in a kinase-dead version of the full length kinase [Genes Dev. 1996; 10 (18):2251-64]. It has been reported that HPK1 inhibition in HPKI kinase-dead knock-in mice, when treated with anti-PD-1 or anti-PDL1 antibodies demonstrate enhanced efficacy in colon cancer models relative to anti-PD-1 or anti-PDL1 treatment alone (Cell Reports 2018, 25, 80-94, and PCT Patent Application Publication Nos. WO2016/205942 and WO2016/090300). Combining or sequencing immunotherapies that target distinct immune pathways is therefore a rational strategy to increase the magnitude of the antitumor immune response over that generated with a single agent.

HPK1 plays significant roles in regulating lymphocyte receptor signaling and function. Moreover, the restricted expression of HPK1 in hematopoietic cells and the roles of HPK1 in immune cells suggest that HPK1 would be an ideal drug target for enhancing antitumor immunity. Furthermore, data from preclinical studies suggest that gene-targeted disruption of HPK1 can promote the proliferation, survival, and function of various immune cells [e.g., T cells, NK cells, and dendritic cells (DC)], and synergistically inhibit tumor growth with anti-PD-1/PDL-1 mAb. Support for this strong rationale was evident by some reports in the literature that HPK1 kinase-dead knock-in mouse bearing colorectal tumors (MC38) showed significant growth arrest treated with an anti-PD1 or anti-PDL1 antibody (PCT Patent Application Publication No. WO2016/090300). Thus, combining a small molecule that inhibits HPK1 with another immunotherapy would appear to be a rational and more effective approach toward treating cancers.

Inhibiting kinases such as HPK1 therefore represents promising targets for immunooncology due to their role in limiting T-cell activation. At the same time, in the pursuit of these targets it is desirable that there be selectivity against other kinases that are involved in a robust T cell activation. An example of such a kinase includes, but is not limited to Lck (Sawasdikosol, et. al. Structure 27, 2019, 1-3).

### SUMMARY

Current cancer immunotherapy strategies seek to reverse immune tolerance either by modulating T cell co-receptor signals or boosting the recognition of tumor-associated antigens by using native biomolecules or mAbs. Selective HPK1 inhibitors, combined with other immuno-modulating agents should amplify the anti-tumor activity of immune cells. The present application discloses novel compounds that have such activity.

Accordingly, the application includes a compound of Formula (I), or a pharmaceutically acceptable salt, solvate, and/or prodrug thereof,

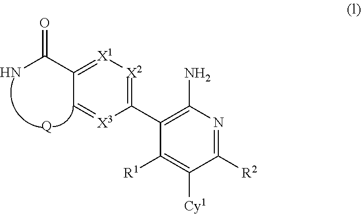

wherein:
X¹ is selected from N and CR³;
X² and X³ are independently selected from N and CR⁴;
Q is C₁₋₄alkylene optionally interrupted by a heteromoiety selected from O, S, S(O), SO₂, and NR⁵ and/or optionally substituted with one or more of R⁶ and/or optionally disubstituted on one carbon with R^{6a} and R^{6b}; provided that when Q comprises the heteromoiety the heteromoiety is separated from the ring amide NH by other than methylene; or
Q is C₂₋₄alkenylene optionally substituted with one or more of R^{6c}; or
Q is selected from C═N and N═C, and is optionally substituted with R^{6c};
R¹ and R² are independently selected from H and halo, provided at least one of R¹ and R² is halo;
R³ is selected from H, halo, OR^{5a}, NR⁷R⁸, C₁₋₆alkyleneNR⁷R⁸ and C₁₋₆alkyl;
each R⁴is independently selected from H, halo, and C₁₋₆alkyl;
R⁵ and R^{5a} are independently selected from H and C₁₋₆alkyl;
each R⁶ is independently selected from ═O, halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR^{7a}R^{8a}, and C₁₋₆alkyleneNR⁷R^{8a};
R^{6a} and R^{6b} are joined to form, together with the carbon atom therebetween, a 3- to 6-membered, saturated or unsaturated ring optionally containing one heteromoiety selected from N, NH, NC₁₋₆alkyl, O, S, S(O), and SO₂ and optionally substituted with one or more of halo and C₁₋₆alkyl;
each R^{6c} is independently selected from halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl NR^{7a}R^{8a}, and C₁₋₆alkyleneNR^{7a}R^{8a};
R⁷, R^{7a}, R⁸, and R^{8a} are each independently selected from H and C₁₋₆alkyl; or
R⁷ and R⁸ or R^{7a} and R^{8a} are joined to form, together with the nitrogen atom therebetween, a 3- to 7-membered saturated or unsaturated ring, optionally containing one additional heteromoiety selected from N, NH, NC₁₋₆alkyl, O, S, S(O), and SO₂ and optionally substituted with one or more of halo and C₁₋₆alkyl;
Cy¹ is C₆₋₂₀aryl or C₅₋₂₀heteroaryl, and Cy¹ is unsubstituted or is substituted with one or more independently selected R⁹ groups or
Cy¹ is substituted with Z-Cy², or
Cy¹ is substituted with Z-Cy² and one or more independently selected R⁹;
each R⁹ is independently selected from halo, ═O, CN, NO₂, C₁₋₆alkyl, C₂₋₆alkenyl, C₂₋₆alkynyl, OR¹⁰, C(O)R¹⁰, CO₂R¹⁰, P(O)R¹⁰R¹¹, P(O)(OR¹⁰)(OR¹¹), SR¹⁰, S(O)R¹⁰, SO₂R¹⁰, S(O)(═NR¹¹)R¹⁰, SO₂NR¹⁰R¹¹, SiR¹²R^{12a}R^{12b}, C₁₋₆alkyleneOR¹⁰, OC₁₋₆alkyleneOR¹⁰, C₁₋₆alkyleneNR^{10a}R^{11a}, OC₁₋₆alkyleneNR¹⁰R¹¹, NR¹¹C₁₋₆alkyleneNR¹⁰R¹¹, NR¹¹C₁₋₆alkyleneOR¹⁰, NR^{10a}R^{11a} NR¹¹COR¹⁰, NR¹¹CO₂R¹⁰, NR¹¹SO₂R¹⁰, C₃₋₇cycloalkyl, C₃₋₇heterocycloalkyl, C₁₋₆alkyleneC₃₋₇cycloalkyl and C₁₋₆alkyleneC₃₋₇heterocycloalkyl, the latter four groups being optionally substituted with one or more of R¹³;
R¹⁰ is selected from H, C₁₋₆alkyl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneOR¹⁴, and C₁₋₆alkyleneNR^{14a}R^{14b}, and all alkyl, alkenyl, alkynyl, alkylene, heterocycloalkyl and cycloalkyl groups of R¹⁰ are optionally substituted with one or more of R¹⁵;
R¹¹ is selected from H and C₁₋₆alkyl; or
R¹⁰ and R¹¹ are joined to form, together with the atom(s) therebetween, a 4- to 6-membered saturated or unsaturated ring, optionally containing one additional heteromoiety selected from N, NR¹⁶, O, S, S(O), and SO₂, and optionally substituted with one or more of R¹⁵;
R^{10a} is selected from H, C₁₋₆alkyl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneOR¹⁴, and C₁₋₆alkyleneNR^{14a}R^{14b}, and all alkyl, alkenyl, alkynyl, alkylene, heterocycloalkyl and cycloalkyl groups of R^{10a} are optionally substituted with one or more of R¹⁵;
R^{11a} is selected from H and C₁₋₆alkyl;
R¹², R^{12a} and R^{12b} are independently selected from OR¹⁷, C₁₋₆alkyl, C₃₋₁₁cycloalkyl, C₃₋₁₀heterocycloalkyl, C₁₋₆alkyleneC₃₋₁₀cycloalkyl, and C₁₋₆alkyleneC₃₋₁₀heterocycloalkyl;
each R¹³ is independently selected from halo, OH, C₁₋₆alkyl, OC₁₋₆alkyl, CN and NR^{13a}R^{13b}R^{13a} and R^{13b} are each independently selected from H and C₁₋₆alkyl;
R¹⁴, R^{14a} and R^{14b} are independently selected from H and C₁₋₆alkyl;
each R¹⁵ is independently selected from halo, C₁₋₆alkyl, CN and NR^{15a}R^{15b};
R^{15a} and R^{15b} are each independently selected from H and C₁₋₆alkyl;
R¹⁶ is selected from H and C₁₋₆alkyl;
R¹⁷ is selected from H, C₁₋₆alkyl, C₃₋₁₁cycloalkyl, C₃₋₁₀heterocycloalkyl, C₁₋₆alkyleneC₃₋₁₀cycloalkyl, C₁₋₆alkyleneC₃₋₁₀heterocycloalkyl C₁₋₆alkyleneOR¹⁸, and C₁₋₆alkyleneNR¹⁸R¹⁹;
R¹⁸ and R¹⁹ are independently selected from H and C₁₋₆alkyl;
Z is absent, or is selected from C₁₋₆alkylene, O, C(O), CO₂, S, S(O), SO₂, S(O)(═NR^{11b}), NR^{11b}, C₁₋₆alkyleneO, C₁₋₆alkyleneC(O), C₁₋₆alkyleneCO₂, C₁₋₆alkyleneS, C₁₋₆alkyleneS(O), C₁₋₆alkyleneSO₂, C₁₋₆alkyleneS(O)(═NR^{11b}), C₁₋₆alkyleneNR^{11b}, OC₁₋₆alkylene, C(O)C₁₋₆alkylene, CO₂C₁₋₆alkylene, SC₁₋₆alkylene, S(O)C₁₋₆alkylene, SO₂C₁₋₆alkylene, S(O)(═NR^{11b})C₁₋₆alkylene and NR^{11b}C₁₋₆alkylene;
R^{11b} is selected from H and C₁₋₆alkyl;
Cy² is selected from C₃₋₁₄cycloalkyl and C₃₋₁₄heterocycloalkyl, and Cy² is unsubstituted or substituted with one or more independently selected R²⁰;
each R²⁰ is independently selected from halo, ═O, CN, OH, C₁₋₆alkyl, C₂₋₆alkenyl, C₂₋₆alkynyl, C₃₋₁₀cycloalkyl, C₃₋₁₀heterocycloalkyl, C₆₋₁₁aryl, C₅₋₁₄heteroaryl, C₁₋₆alkyleneC₃₋₁₀cycloalkyl, C₁₋₆alkyleneC₃₋₁₀heterocycloalkyl, C₁₋₆alkyleneC₆₋₁₁aryl, C₁₋₆alkyleneC₅₋₁₄heteroaryl, OC₁₋₆alkyl, OC₂₋₆alkenyl, OC₂₋₆alkynyl, C₁₋₆alkyleneOR²¹, OC₁₋₆alkyleneOR²¹, C₁₋₆alkyleneNR²²R²³, OC₁₋₆alkyleneNR²²R²³, SC₁₋₆alkyl, SC₂₋₆alkenyl, SC₂₋₆alkynyl, C(O)C₁₋₆alkyl, C(O)C₂₋₆alkenyl, C(O)C₂₋₆alkynyl, C(O)C₃₋₁₀cycloalkyl, C(O)C₃₋₁₀heterocycloalkyl, C(O)C₆₋₁₁aryl, C(O)C₅₋₁₄heteroaryl, C(O)C₁₋₆alkyleneC₃₋₁₀cycloalkyl, C(O)C₁₋₆alkyleneC₃₋₁₀heterocycloalkyl, C(O)C₁₋₆alkyleneC₆₋₁₁aryl, C(O)C₁₋₆alkyleneC₅₋₁₄heteroaryl, C(O)C₁₋₆alkylenylOR²¹, C(O)C₁₋₆alkyleneNR²²R²³, C(O)C₁₋₆alkyleneOC₁₋₆alkyleneNR²²R²³, C(O)NR²²R²³, CO₂C₁₋₆alkyl, CO₂C₂₋₆alkenyl, CO₂C₂₋₆alkynyl, CO₂C₁₋₆alkyleneOR²¹, CO₂C₁₋₆alkyleneOC₁₋₆alkyleneNR²²R²³, NR²²R²³, NR²⁴C₃₋₁₀cycloalkyl, NR²⁴C₃₋₁₀heterocycloalkyl, NR²⁴C₆₋₁₁aryl, NR²⁴C₅₋₁₄heteroaryl NR²⁴C₁₋₆alkyleneOR²¹, NR²⁴C₁₋₆alkyleneNR²²R²³, NR²⁴C₁₋₆alkyleneC₃₋₁₀cycloalkyl, NR²⁴C₁₋₆alkyleneC₃₋₁₀heterocycloalkyl, NR²⁴C₁₋₆alkyleneC₆₋₁₁aryl, NR²⁴C₁₋₆alkyleneC₅₋₁₄heteroaryl NR²⁴SO₂C₁₋₆alkyl, SO₂C₁₋₆alkyl, SO₂C₂₋₆alkenyl, SO₂C₂₋₆alkynyl, and SO₂NR²²R²³, and alkyl, alkenyl, alkynyl, alkylene, aryl, heteroaryl, heterocycloalkyl and cycloalkyl groups of R²⁰ are optionally substituted with one or more of R25
R²¹ is selected from H, C₁₋₆alkyl, C₁₋₆alkyleneOC₁₋₆alkyl, C₃₋₁₁cycloalkyl, C₃₋₁₀heterocycloalkyl, C₆₋₁₁aryl, C₅₋₁₄heteroaryl, C₂₋₆alkenyl, C₂₋₆alkynyl, C₁₋₆alkyleneC₃₋₁₀cycloalkyl, C₁₋₆alkyleneC₃₋ₗ₀heterocycloalkyl, C₁₋₆alkyleneC₆₋₁₁aryl and C₁₋₆alkyleneC₅₋₁₄heteroaryl;
R²² is selected from H and C₁₋₆alkyl;
R²³ is selected from H, C₁₋₆alkyl, C₁₋₆alkyleneOC₁₋₆alkyl, C₃₋₁₀cycloalkyl, C₃₋₁₀heterocycloalkyl, C₆₋₁₁aryl and C₅₋₁₄heteroaryl C₁₋₆alkyleneC₃₋₁₀cycloalkyl, C₁₋₆alkyleneC₃₋₁₀heterocycloalkyl, C₁₋₆alkyleneC₆₋₁₁aryl and C₁₋₆alkyleneC₅₋₁₄heteroaryl, or R²² and R²³ are joined to form, together with the nitrogen atom therebetween, a 4- to 6-membered saturated or unsaturated ring, optionally containing one additional heteromoiety selected from N, NR²⁶, O, S, S(O), and SO₂, and optionally substituted with one or more of halo and C₁₋₆alkyl;
R²⁴ is selected from H and C₁₋₆alkyl;
each R²⁵ is independently selected from halo, C₁₋₆alkyl, CN and NR^{25a}R^{25b}R^{25a} and R^{25b} are each independently selected from H and C₁₋₆alkyl; and
R²⁶ is selected from H and C₁₋₆alkyl;
wherein all available hydrogen atoms are optionally substituted with a fluorine atom.

The present application also includes a composition comprising one or more compounds of the application and a carrier. In an embodiment, the composition is a pharmaceutical composition comprising one or more compounds of the application and a pharmaceutically acceptable carrier.

In an embodiment, the compounds of the application are used as medicaments. Accordingly, the application also includes a compound of the application for use as a medicament.

The compounds of the application have been shown to inhibit HPK1. Therefore the compounds of the application are useful for treating diseases, disorders or conditions that are treatable by inhibiting HPK1 activity. Accordingly, the present application also includes a method of treating a disease, disorder or condition that is treatable by inhibiting HPK1, comprising administering a therapeutically effective amount of one or more compounds of the application to a subject in need thereof.

The present application also includes a use of one or more compounds of the application for treatment of a disease, disorder or condition that is treatable by inhibiting HPK1, as well as a use of one or more compounds of the application for the preparation of a medicament for treatment of a disease, disorder or condition that is treatable by inhibiting HPK1. The application further includes one or more compounds of the application for use in treating a disease, disorder or condition that is treatable by inhibiting HPK1.

In a further embodiment, the disease, disorder or condition that is treatable by inhibiting HPK1 is cancer and the one or more compounds of the application are administered in combination with one or more additional cancer treatments. In another embodiment, the additional cancer treatment is selected from radiotherapy, chemotherapy, targeted therapies such as antibody therapies and small molecule therapies such as tyrosine-kinase inhibitors, immunotherapy, hormonal therapy and anti-angiogenic therapies. In another embodiment, the additional cancer treatment is selected from an antibody that binds to PD-1 or PD-1.

The application additionally provides a process for the preparation of compounds of the application. General and specific processes are discussed in more detail and set forth in the Examples below.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component or effect, such as an additional or second compound, the second compound as used herein is different from the other compounds or first compound. A “third” compound is different from the other, first, and second compounds, and further enumerated or “additional” compounds are similarly different.

The term “and/or” as used herein means that the listed items are present, or used, individually or in combination. In effect, this term means that “at least one of” or “one or more” of the listed items is used or present. The term “and/or” with respect to enantiomers, prodrugs, salts and/or solvates thereof means that the compounds of the application exist as individual enantiomers, prodrugs, salts and hydrates, as well as a combination of, for example, a salt of a solvate of a compound of the application.

The term “compound of the application” or “compound of the present application” and the like as used herein refers to a compound of Formula (I) or Formula I-A, I-B, I-C, I-D, I-E, I-F, I-G, I-H, I-J, I-K, I-L, I-M, I-N, I-O, I-P, I-Q, I-R, I-S, I-T and I-U or salts, solvates and/or prodrugs thereof.

The term “composition of the application” or “composition of the present application” and the like as used herein refers to a composition comprising one or more compounds of the application.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

The present description refers to a number of chemical terms and abbreviations used by those skilled in the art. Nevertheless, definitions of selected terms are provided for clarity and consistency.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “inert organic solvent” as used herein refers to a solvent that is generally considered as non-reactive with the functional groups that are present in the compounds to be combined together in any given reaction so that it does not interfere with or inhibit the desired synthetic transformation. Organic solvents are typically non-polar and dissolve compounds that are non soluble in aqueous solutions.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms. All alkyl groups are optionally fluoro-substituted unless otherwise indicated.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms. All alkylene groups are optionally fluoro-substituted unless otherwise indicated.

The term “alkenyl” as used herein, whether it is used alone or as part of another group, means straight or branched chain, unsaturated alkyl groups containing at least one double bond. The number of carbon atoms that are possible in the referenced alkylene group are indicated by the prefix “Cₙ₁₋ₙ₂”. For example, the term C₂₋₆alkenyl means an alkenyl group having 2, 3, 4, 5 or 6 carbon atoms and at least one double bond. All alkenyl groups are optionally fluoro-substituted unless otherwise indicated.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms. All alkynyl groups are optionally fluoro-substituted unless otherwise indicated.

The term “cycloalkyl,” as used herein, whether it is used alone or as part of another group, means a saturated carbocyclic group containing from 3 to 20 carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms. All cycloalkyl groups are optionally fluoro-substituted unless otherwise indicated.

The term “aryl” as used herein, whether it is used alone or as part of another group, refers to carbocyclic groups containing at least one aromatic ring and contains 6 to 20 carbon atoms. All aryl groups are optionally fluoro-substituted unless otherwise indicated.

The term “heterocycloalkyl” as used herein, whether it is used alone or as part of another group, refers to cyclic groups containing at least one non-aromatic ring containing from 3 to 20 atoms in which one or more of the atoms are a heteroatom selected from O, S and N and the remaining atoms are C. Heterocycloalkyl groups are either saturated or unsaturated (i.e. contain one or more double bonds). When a heterocycloalkyl group contains the prefix Cₙ₁₋ₙ₂ this prefix indicates the number of carbon atoms in the corresponding carbocyclic group, in which one or more, suitably 1 to 5, of the ring atoms is replaced with a heteroatom as selected from O, S and N and the remaining atoms are C. Heterocycloalkyl groups are optionally benzofused. The heteroatom may be substituted or oxidized where valency allows. All heterocycloalkyl groups are optionally fluoro-substituted unless otherwise indicated.

The term “heteroaryl” as used herein, whether it is used alone or as part of another group, refers to cyclic groups containing at least one heteroaromatic ring containing 5-20 atoms in which one or more of the atoms are a heteroatom selected from O, S and N and the remaining atoms are C. When a heteroaryl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above. Heteroaryl groups are optionally benzofused. The heteroatom may be substituted or oxidized where valency allows. All heteroaryl groups are optionally fluoro-substituted unless otherwise indicated.

All cyclic groups, including aryl, heteroaryl, heterocycloalkyl, and cycloalkyl groups, contain one or more than one ring (i.e. are polycyclic). When a cyclic group contains more than one ring, the rings may be fused, bridged, and/or spirofused.

The term “benzofused” as used herein refers to a polycyclic group in which a benzene ring is fused with another ring.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “fluoro-substituted” refers to the substitution of one or more, including all, available hydrogens in a referenced group with fluoro.

The terms “halo” or “halogen” as used herein, whether it is used alone or as part of another group, refers to a halogen atom and includes fluoro, chloro, bromo and iodo.

The term “available”, as in “available hydrogen atoms” or “available atoms” refers to atoms that would be known to a person skilled in the art to be capable of replacement by a substituent, such as a fluorine atom.

When a group is said to be substituted with multiple substituents, said substituents are independently selected therefore can be the same or different.

It is to be clear that all available hydrogen atoms in the compounds of the application, and all embodiments thereof, are optionally substituted with a fluorine atom unless otherwise indicated.

The term “cross-coupling” as used herein refers to chemical reactions in which two different starting materials, each of which is usually endowed with an activating group, are reacted together with the aid of a metal catalyst. The result is the loss of the two activating groups and the formation of a new covalent bond between the remaining fragments.

The term “cell” as used herein refers to a single cell or a plurality of cells and includes a cell either in a cell culture or in a subject.

The term “subject” as used herein includes all members of the animal kingdom including mammals, and suitably refers to humans. Thus the methods and uses of the present application are applicable to both human therapy and veterinary applications.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

The term “solvate” as used herein means a compound, or a salt and/or prodrug of a compound, wherein molecules of a suitable solvent are incorporated in the crystal lattice. A suitable solvent is physiologically tolerable at the dosage administered.

The term “prodrug” as used herein means a compound, or salt and/or solvate of a compound, that, after administration, is converted into an active drug.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations.

“Palliating” a disease or disorder means that the extent and/or undesirable clinical manifestations of a disorder or a disease state are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition treatable by inhibiting HPK1, or manifesting a symptom associated with a disease, disorder or condition treatable by inhibition of HPK1.

As used herein, the term “effective amount” or “therapeutically effective amount” means an amount of a compound, or one or more compounds, of the application that is effective, at dosages and for periods of time necessary to achieve the desired result.

The expression “inhibiting HPK1” as used herein refers to inhibiting, blocking and/or disrupting HPK1 enzymatic activity in a cell, in particular a T-cell or B-cell. The inhibiting, blocking and/or disrupting causes a therapeutic effect in the cell.

By “inhibiting, blocking and/or disrupting” it is meant any detectable inhibition, block and/or disruption in the presence of a compound compared to otherwise the same conditions, except for in the absence in the compound.

The term “disease, disorder or condition treatable by inhibiting HPK1” means that the disease, disorder or condition to be treated is affected by, modulated by and/or has some biological basis, either direct or indirect, that includes HPK1 activity, in particular, increased HPK1 activity. These diseases respond favourably when HPK1 activity associated with the disease, disorder or condition is inhibited by one or more of the compounds or compositions of the application.

The term “HPK1” as used herein refers to the hematopoetic progenitor kinase 1.

The term “administered” as used herein means administration of a therapeutically effective amount of a compound, or one or more compounds, or a composition of the application to a cell either in cell culture or in a subject.

The term “neoplastic disorder” as used herein refers to a disease, disorder or condition characterized by cells that have the capacity for autonomous growth or replication, e.g., an abnormal state or condition characterized by proliferative cell growth. The term “neoplasm” as used herein refers to a mass of tissue resulting from the abnormal growth and/or division of cells in a subject having a neoplastic disorder. Neoplasms can be benign (such as uterine fibroids and melanocytic nevi), potentially malignant (such as carcinoma in situ) or malignant (i.e. cancer).

The term “cancer” as used herein refers to cellular-proliferative disease states.

As used herein, the term “effective amount” means an amount effective, at dosages and for periods of time, necessary to achieve a desired result.

### II. Compounds and Compositions

The present application describes a novel class of halo-substituted heterocyclic HPK1 inhibitors.

Accordingly, the application includes a compound of Formula (I) or a pharmaceutically acceptable salt, solvate and/or prodrug thereof,

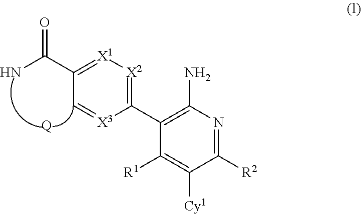

wherein:
X¹ is selected from N and CR³;
X² and X³ are independently selected from N and CR⁴;
Q is C₁₋₄alkylene optionally interrupted by a heteromoiety selected from O, S, S(O), SO₂, and NR⁵ and/or optionally substituted with one or more of R⁶ and/or optionally disubstituted on one carbon with R^{6a} and R^{6b}; provided that when Q comprises the heteromoiety the heteromoiety is separated from the ring amide NH by other than methylene; or
Q is C₂₋₄alkenylene optionally substituted with one or more of R^{6c}; or
Q is selected from C═N and N═C, and is optionally substituted with R^{6c};
R¹ and R² are independently selected from H and halo, provided at least one of R¹ and R² is halo;
R³ is selected from H, halo, OR^{5a}, NR⁷R⁸, C₁₋₆alkyleneNR⁷R⁸ and C₁₋₆alkyl;
R⁴is selected from H, halo, and C₁₋₆alkyl;
R⁵ and R^{5a} are independently selected from H and C₁₋₆alkyl;
each R⁶ is independently selected from ═O, halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR^{7a}R^{8a}, and C₁₋₆alkyleneNR^{7a}R^{8a};
R^{6a} and R^{6b} are joined to form, together with the carbon atom therebetween, a 3- to 6-membered, saturated or unsaturated ring optionally containing one heteromoiety selected from N, NH, NC₁₋₆alkyl, O, S, S(O), and SO₂ and optionally substituted with one or more of halo and C₁₋₆alkyl;
each R^{6c} is independently selected from halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl NR^{7a}R^{8a}, and C₁₋₆alkyleneNR^{7a}R^{8a};
R⁷, R^{7a}, R⁸, and R^{8a} are each independently selected from H and C₁₋₆alkyl; or
R⁷ and R⁸ or R^{7a} and R^{8a} are joined to form, together with the nitrogen atom therebetween, a 3- to 7-membered saturated or unsaturated ring, optionally containing one additional heteromoiety selected from N, NH, NC₁₋₆alkyl, O, S, S(O), and SO₂ and optionally substituted with one or more of halo and C₁₋₆alkyl;
Cy¹ is C₆₋₂₀aryl or C₅₋₂₀heteroaryl, and Cy¹ is unsubstituted or is substituted with one or more of R⁹, or
Cy¹ is substituted with Z-Cy², or
Cy¹ is substituted with Z-Cy² and one or more independently selected R⁹;
each R⁹ is independently selected from halo, ═O, CN, NO₂, C₁₋₆alkyl, C₂₋₆alkenyl, C₂₋₆alkynyl, OR¹⁰, C(O)R¹⁰, CO₂R¹⁰, P(O)R¹⁰R¹¹, P(O)(OR¹⁰)(OR¹¹), SR¹⁰, S(O)R¹⁰, SO₂R¹⁰, S(O)(═NR¹¹)R¹⁰, SO₂NR¹⁰R¹¹, SiR¹²R^{12a}R^{12b}, C₁₋₆alkyleneOR¹⁰, OC₁₋₆alkyleneOR¹⁰, C₁₋₆alkyleneNR^{10a}R^{11a}, OC₁₋₆alkyleneNR¹⁰R¹¹, NR¹¹C₁₋₆alkyleneNR¹⁰R¹¹, NR¹¹C₁₋₆alkyleneOR¹⁰, NR^{10a}R^{11a} NR¹¹COR¹⁰, NR¹¹CO₂R¹⁰, NR¹¹SO₂R¹⁰, C₃₋₇cycloalkyl, C₃₋₇heterocycloalkyl, C₁₋₆alkyleneC₃₋₇cycloalkyl and C₁₋₆alkyleneC₃₋₇heterocycloalkyl, the latter four groups being optionally substituted with one or more of R¹³;
R¹⁰ is selected from H, C₁₋₆alkyl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneOR¹⁴, and C₁₋₆alkyleneNR^{14a}R^{14b}, and all alkyl, alkenyl, alkynyl, alkylene, heterocycloalkyl and cycloalkyl groups of R¹⁰ are optionally substituted with one or more of R¹⁵;
R¹¹ is selected from H and C₁₋₆alkyl; or
R¹⁰ and R¹¹ are joined to form, together with the atom(s) therebetween, a 4- to 6-membered saturated or unsaturated ring, optionally containing one additional heteromoiety selected from N, NR¹⁶, O, S, S(O), and SO₂, and optionally substituted with one or more of R¹⁵;
R^{10a} is selected from H, C₁₋₆alkyl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneOR¹⁴, and C₁₋₆alkyleneNR^{14a}R^{14b}, and all alkyl, alkenyl, alkynyl, alkylene, heterocycloalkyl and cycloalkyl groups of R¹⁰ are optionally substituted with one or more of R¹⁵;
R^{11a} is selected from H and C₁₋₆alkyl;
R¹², R^{12a} and R^{12b} are independently selected from OR¹⁷, C₁₋₆alkyl, C₃₋₁₁cycloalkyl, C₃₋₁₀heterocycloalkyl, C₁₋₆alkyleneC₃₋₁₀cycloalkyl, and C₁₋₆alkyleneC₃₋₁₀heterocycloalkyl;
each R¹³ is independently selected from halo, OH, CN, C₁₋₆alkyl, OC₁₋₆alkyl, and NR^{13a}R^{13b}R^{13a} and R^{13b} are each independently selected from H and C₁₋₆alkyl;
R¹⁴, R^{14a} and R^{14b} are independently selected from H and C₁₋₆alkyl;
each R¹⁵ is independently selected from halo, C₁₋₆alkyl, CN and NR^{15a}R^{15b};
R^{15a} and R^{15b} are each independently selected from H and C₁₋₆alkyl;
R¹⁶ is selected from H and C₁₋₆alkyl;
R¹⁷ is selected from H, C₁₋₆alkyl, C₃₋₁₁cycloalkyl, C₃₋₁₀heterocycloalkyl, C₁₋₆alkyleneC₃₋₁₀cycloalkyl, C₁₋₆alkyleneC₃₋₁₀heterocycloalkyl C₁₋₆alkyleneOR¹⁸, and C₁₋₆alkyleneNR¹⁸R¹⁹;
R¹⁸ and R¹⁹ are independently selected from H and C₁₋₆alkyl;
Z is absent, or is selected from C₁₋₆alkylene, 0, C(O), CO₂, S, S(O), SO₂, S(O)(═NR^{11b}), NR^{11b}, C₁₋₆alkyleneO, C₁₋₆alkyleneC(O), C₁₋₆alkyleneCO₂, C₁₋₆alkyleneS, C₁₋₆alkyleneS(O), C₁₋₆alkyleneSO₂, C₁₋₆alkyleneS(O)(═NR^{11b}), C₁₋₆alkyleneNR^{11b}, OC₁₋₆alkylene, C(O)C₁₋₆alkylene, CO₂C₁₋₆alkylene, SC₁₋₆alkylene, S(O)C₁₋₆alkylene, SO₂C₁₋₆alkylene, S(O)(═NR^{11b})C₁₋₆ alkylene and NR^{11b}C₁₋₆alkylene;
R^{11b} is selected from H and C₁₋₆alkyl;
Cy² is selected from C₃₋₁₄cycloalkyl and C₃₋₁₄heterocycloalkyl, and Cy² is unsubstituted or substituted with one or more independently selected R²⁰ each R²⁰ is independently selected from halo, ═O, CN, OH, C₁₋₆alkyl, C₂₋₆alkenyl, C₂₋₆alkynyl, C₃₋₁₀cycloalkyl, C₃₋₁₀heterocycloalkyl, C₆₋₁₁aryl, C₅₋₁₄heteroaryl, C₁₋₆alkyleneC₃₋₁₀cycloalkyl, C₁₋₆alkyleneC₃₋₁₀heterocycloalkyl, C₁₋₆alkyleneC₆₋₁₁aryl, C₁₋₆alkyleneC₅₋₁₄heteroaryl, OC₁₋₆alkyl, OC₂₋₆alkenyl, OC₂₋₆alkynyl, C₁₋₆alkyleneOR²¹, OC₁₋₆alkyleneOR²¹, C₁₋₆alkyleneNR²²R²³, OC₁₋₆alkyleneNR²²R²³, SC₁₋₆alkyl, SC₂₋₆alkenyl, SC₂₋₆alkynyl, C(O)C₁₋₆alkyl, C(O)C₂₋₆alkenyl, C(O)C₂₋₆alkynyl, C(O)C₃₋₁₀cycloalkyl, C(O)C₃₋₁₀heterocycloalkyl, C₆₋₁₁ aryl, C₅₋₁₄heteroaryl, C(O)C₁₋₆alkyleneC₃₋₁₀cycloalkyl, C(O)C₁₋₆alkyleneC₃₋₁₀heterocycloalkyl, C(O)C₁₋₆alkyleneC₆₋₁₁aryl, C(O)C₁₋₆alkyleneC₅₋₁₄heteroaryl, C(O)C₁₋₆alkylenylOR²¹, C(O)C₁₋₆alkyleneNR²²R²³, C(O)C₁₋₆alkyleneOC₁₋₆alkyleneNR²²R²³, C(O)NR²²R²³, CO₂C₁₋₆alkyl, CO₂C₂₋₆alkenyl, CO₂C₂₋₆alkynyl, CO₂C₁₋₆alkyleneOR²¹, CO₂C₁₋₆alkyleneOC₁₋₆alkyleneNR²²R²³, NR²²R²³, NR²⁴C₃₋₁₀cycloalkyl, NR²⁴C₃₋₁₀heterocycloalkyl, NR²⁴C₆₋₁₁aryl, NR²⁴C₅₋₁₄heteroaryl NR²⁴C₁₋₆alkyleneOR²¹, NR²⁴C₁₋₆alkyleneNR²²R²³, NR²⁴C₁₋₆alkyleneC₃₋₁₀cycloalkyl, NR²⁴C₁₋₆alkyleneC₃₋₁₀heterocycloalkyl, NR²⁴C₁₋₆alkyleneC₆₋₁₁aryl, NR²⁴C₁₋₆alkyleneC₅₋₁₄heteroaryl NR²⁴SO₂C₁₋₆alkyl, SO₂C₁₋₆alkyl, SO₂C₂₋₆alkenyl, SO₂C₂₋₆alkynyl, and SO₂NR²²R²³, and alkyl, alkenyl, alkynyl, alkylene, aryl, heteroaryl, heterocycloalkyl and cycloalkyl groups of R²⁰ are optionally substituted with one or more of R²⁵;
R²¹ is selected from H, C₁₋₆alkyl, C₁₋₆alkyleneOC₁₋₆alkyl, C₃₋₁₁cycloalkyl, C₃₋₁₀heterocycloalkyl, C₆₋₁₁aryl, heteroaryl C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneC₆₋₁₁aryl and C₁₋₆alkyleneC₅₋₁₄heteroaryl;
R²² is selected from H and C₁₋₆alkyl;
R²³ is selected from H, C₁₋₆alkyl, C₁₋₆alkyleneOC₁₋₆alkyl, C₃₋₁₀cycloalkyl, C₃₋₁₀heterocycloalkyl, C₆₋₁₁aryl, heteroaryl C₁₋₆alkyleneC₃₋₁₀cycloalkyl, C₁₋₆alkyleneC₃₋₁₀heterocycloalkyl, C₁₋₆alkyleneC₆₋₁₁aryl and C₁₋₆alkyleneC₅₋₁₄heteroaryl, or
R²² and R²³ are joined to form, together with the nitrogen atom therebetween, a 4- to 6-membered saturated or unsaturated ring, optionally containing one additional heteromoiety selected from N, NR²⁶, O, S, S(O), and SO₂, and optionally substituted with one or more of halo and C₁₋₆alkyl;
R²⁴ is selected from H and C₁₋₆alkyl;
each R²⁵ is independently selected from halo, C₁₋₆alkyl, CN and NR^{25a}R^{25b};
R^{25a} and R^{25b} are each independently selected from H and C₁₋₆alkyl; and
R²⁶ is selected from H and C₁₋₆alkyl;
wherein all available hydrogen atoms are optionally substituted with a fluorine atom.

The application also includes a compound of Formula (I) or a pharmaceutically acceptable salt, solvate and/or prodrug thereof,

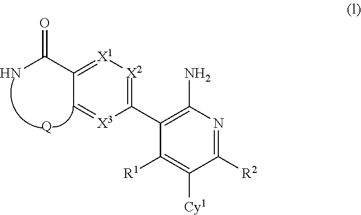

wherein:
X¹ is selected from N and CR³;
X² and X³ are independently selected from N and CR⁴;
Q is C₁₋₄alkylene optionally interrupted by a heteromoiety selected from O, S, S(O), SO₂, and NR⁵ and/or optionally substituted with one or more of R⁶ and/or optionally disubstituted on one carbon with R^{6a} and R^{6b}; provided that when Q comprises the heteromoiety the heteromoiety is separated from the ring amide NH by other than methylene; or
Q is C₂₋₄alkenylene optionally substituted with one or more of R^{6c}; or
Q is selected from C═N and N═C, and is optionally substituted with R^{6c};
R¹ and R² are independently selected from H and halo, provided at least one of R¹ and R² is halo;
R³ is selected from H, halo, OR^{5a}, NR⁷R⁸, C₁₋₆alkyleneNR⁷R⁸ and C₁₋₆alkyl;
R⁴ is selected from H, halo, and C₁₋₆alkyl;
R⁵ and R^{5a} are independently selected from H and C₁₋₆alkyl;
each R⁶ is independently selected from ═O, halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR^{7a}R^{8a}, and C₁₋₆alkyleneNR^{7a}R^{8a};
R^{6a} and R^{6b} are joined to form, together with the atom therebetween, a 3- to 6-membered, saturated or unsaturated ring optionally containing one additional heteromoiety selected from N, NH, NC₁₋₆alkyl, O, S, S(O), and SO₂ and optionally substituted with one or more of halo and C₁₋₆alkyl;
each R^{6c} is independently selected from halo, C₁₋₆alkyl, C₃₋₆cycloalkyl, C₃₋₆heterocycloalkyl, C₁₋₆alkyleneC₃₋₆cycloalkyl and C₁₋₆alkyleneC₃₋₆heterocycloalkyl, OH, OC₁₋₆alkyl NR^{7a}R^{8a}, and C₁₋₆alkyleneNR^{7a}R^{8a};
R⁷, R^{7a}, R⁸, and R^{8a} are each independently selected from H and C₁₋₆alkyl; or
R⁷ and R⁸ or R^{7a} and R^{8a} are joined to form, together with the atom therebetween, a 3- to 7-membered saturated or unsaturated ring, optionally containing one additional heteromoiety selected from N, NH, NC₁₋₆alkyl, O, S, S(O), and SO₂ and optionally substituted with one or more of halo and C₁₋₆alkyl;
Cy¹ is C₆₋₂₀aryl or C₅₋₂₀heteroaryl, and Cy¹ is unsubstituted or is substituted with one or more of R⁹, or
Cy¹ is substituted with Z-Cy², or
Cy¹ is substituted with Z-Cy² and one or more independently selected R⁹;
each R⁹ is independently selected from halo, ═O, CN, NO₂, C₁₋₆alkyl, C₂₋₆alkenyl, C₂₋₆alkynyl, OR¹⁰, C(O)R¹⁰, CO₂R¹⁰, P(O)R¹⁰R¹¹, P(O)(OR¹⁰)(OR¹¹), SR¹⁰, S(O)R¹⁰, SO₂R¹⁰, S(O)(═NR¹¹)R¹⁰, SO₂NR¹⁰R¹¹, SiR¹²R^{12a}R^{12b}, C₁₋₆alkyleneOR¹⁰, OC₁₋₆alkyleneOR¹⁰, C₁₋₆alkyleneNR^{10a}R^{11a}, OC₁₋₆alkyleneNR¹⁰R¹¹, NR¹¹C₁₋₆alkyleneNR¹⁰R¹¹, NR¹¹C₁₋₆alkyleneOR¹⁰, NR^{10a}R^{11a} NR¹¹COR¹⁰, NR¹¹CO₂R¹⁰, NR¹¹SO₂R¹⁰, C₃₋₇cycloalkyl, C₃₋₇heterocycloalkyl, C₁₋₆alkyleneC₃₋₇cycloalkyl and C₁₋₆alkyleneC₃₋₇heterocycloalkyl, the latter four groups being optionally substituted with one or more of R¹³;
R¹⁰ is selected from H, C₁₋₆alkyl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneOR¹⁴, and C₁₋₆alkyleneNR^{14a}R^{14b}, and all alkyl, alkenyl, alkynyl, alkylene, and cycloalkyl groups of R¹⁰ are optionally substituted with one or more of R¹⁵;
R¹¹ is selected from H and C₁₋₆alkyl; or
R¹⁰ and R¹¹ are joined to form, together with the atom therebetween, a 4- to 6-membered saturated or unsaturated ring, optionally containing one additional heteromoiety selected from N, NR¹⁶, O, S, S(O), and SO₂, and optionally substituted with one or more of R¹⁵;
R^{10a} is selected from H, C₁₋₆alkyl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneOR¹⁴, and C₁₋₆alkyleneNR^{14a}R^{14b}, and all alkyl, alkenyl, alkynyl, alkylene, and cycloalkyl groups of R^{10a} are optionally substituted with one or more of R¹⁵;
R^{11a} is selected from H and C₁₋₆alkyl;
R¹², R^{12a} and R^{12b} are independently selected from OR¹⁷, C₁₋₆alkyl, C₃₋₁₁cycloalkyl, C₃₋₁₀heterocycloalkyl, C₁₋₆alkyleneC₃₋₁₀cycloalkyl, and C₁₋₆alkyleneC₃₋₁₀heterocycloalkyl;
each R¹³ is independently selected from halo, C₁₋₆alkyl, CN and NR^{13a}R^{13b};
R^{13a} and R^{13b} are each independently selected from H and C₁₋₆alkyl;
R¹⁴, R^{14a} and R^{14b} are independently selected from H and C₁₋₆alkyl;
each R¹⁵ is independently selected from halo, C₁₋₆alkyl, CN and NR^{15a}R^{15b};
R^{15a} and R^{15b} are each independently selected from H and C₁₋₆alkyl;
R¹⁶ is selected from H and C₁₋₆alkyl;
R¹⁷ is selected from H, C₁₋₆alkyl, C₃₋₁₀cycloalkyl, C₃₋₁₀heterocycloalkyl, C₁₋₆alkyleneC₃₋₁₀cycloalkyl, C₁₋₆alkyleneC₃₋₁₀heterocycloalkyl C₁₋₆alkyleneOR¹⁸, and C₁₋₆alkyleneNR¹⁸R¹⁹;
R¹⁸ and R¹⁹ are independently selected from H and C₁₋₆alkyl;
Z is absent, or is selected from C₁₋₆alkylene, O, C(O), CO₂, S, S(O), SO₂, S(O)(═NR^{11b}), and NR^{11b};
R^{11b} is selected from H and C₁₋₆alkyl;
Cy² is selected from C₃₋₁₄cycloalkyl and C₃₋₁₄heterocycloalkyl, and Cy² is unsubstituted or substituted with one or more independently selected R²⁰;
each R²⁰ is independently selected from halo, ═O, CN, OH, C₁₋₆alkyl, C₂₋₆alkenyl, C₂₋₆alkynyl, C₃₋₁₀cycloalkyl, C₃₋₁₀heterocycloalkyl, C₁₋₆alkyleneC₃₋₁₀cycloalkyl, C₁₋₆alkyleneC₃₋₁₀heterocycloalkyl, OC₁₋₆alkyl, OC₂₋₆alkenyl, OC₂₋₆alkynyl, C₁₋₆alkyleneOR²¹, OC₁₋₆alkyleneOR²¹, C₁₋₆alkyleneNR²²R²³, OC₁₋₆alkyleneNR²²R²³, SC₁₋₆alkyl, SC₂₋₆alkenyl, SC₂₋₆alkynyl, C(O)C₁₋₆alkyl, C(O)C₂₋₆alkenyl, C(O)C₂₋₆alkynyl, C(O)C₃₋₁₀cycloalkyl, C(O)C₃₋₁₀heterocycloalkyl, C(O)C₁₋₆alkyleneC₃₋₁₀cycloalkyl, C(O)C₁₋₆alkyleneC₃₋₁₀heterocycloalkyl, C(O)C₁₋₆alkylenylOR²¹, C(O)C₁₋₆alkyleneNR²²R²³, C(O)C₁₋₆alkyleneOC₁₋₆alkyleneNR²²R²³ C(O)NR²²R²³, CO₂C₁₋₆alkyl, CO₂C₂₋₆alkenyl, CO₂C₂₋₆alkynyl, CO₂C₁₋₆alkyleneOR²¹, CO₂C₁₋₆alkyleneOC₁₋₆alkyleneNR²²R²³, NR²²R²³, NR²⁴C₃₋₁₀cycloalkyl, NR²⁴C₃₋₁₀heterocycloalkyl, NR²⁴C₁₋₆alkyleneOR²¹, NR²⁴C₁₋₆alkyleneC₃₋₁₀cycloalkyl, NR²⁴C₁₋₆alkyleneC₃₋ₗ₀heterocycloalkyl, NR²⁴SO₂C₁₋₆alkyl, SO₂C₁₋₆alkyl, SO₂C₂₋₆alkenyl, SO₂C₂₋₆alkynyl, and SO₂NR²²R²³, and alkyl, alkenyl, alkynyl, alkylene and cycloalkyl groups of R²⁰ are optionally substituted with one or more of R²⁵
R²¹ is selected from H, C₁₋₆alkyl, C₁₋₆alkyleneOC₁₋₆alkyl, C₃₋₁₁cycloalkyl, C₃₋₁₀heterocycloalkyl, C₂₋₆alkenyl, C₂₋₆alkynyl, C₁₋₆alkyleneC₃₋₁₀cycloalkyl, and C₁₋₆alkyleneC₃₋₁₀heterocycloalkyl;
R²² is selected from H and C₁₋₆alkyl;
R²³ is selected from H, C₁₋₆alkyl, C₁₋₆alkyleneOC₁₋₆alkyl, C₃₋₁₀cycloalkyl, C₃₋₁₀heterocycloalkyl, C₁₋₆alkyleneC₃₋₁₀cycloalkyl, and C₁₋₆alkyleneC₃₋₁₀heterocycloalkyl, or R²² and R²³ are joined to form, together with the atom therebetween, a 4- to 6-membered saturated or unsaturated ring, optionally containing one additional heteromoiety selected from N, NR²⁶, O, S, S(O), and SO₂, and optionally substituted with one or more of halo and C₁₋₆alkyl;
R²⁴ is selected from H and C₁₋₆alkyl;
each R²⁵ is independently selected from halo, C₁₋₆alkyl, CN and NR^{25a}R^{25b}R^{25a} and R^{25b} are each independently selected from H and C₁₋₆alkyl; and
R²⁶ is selected from H and C₁₋₆alkyl;
wherein all available hydrogen atoms are optionally substituted with a fluorine atom.

In all embodiments below it is to be understood that all available hydrogen atoms are optionally substituted with a fluorine atom. This has not been repeated throughout. Thus in each embodiment where a group is listed that comprises available hydrogen atoms, it is to be understood that all such atoms are optionally replaced with fluorine atoms, for example each recitation of C₁₋₆alkyl is also a recitation of C₁₋₆fluoroalkyl, unless stated otherwise.

In an embodiment, X¹ is N.

In an embodiment, X¹ is CR³.

In an embodiment, R³ is selected from H, F, Cl, OR^{5a}, NR⁷R⁸, C₁₋₄alkyleneNR^{7a}R^{8a} and C₁₋₄alkyl.

In an embodiment, R³ is selected from H, F, Cl, OR^{5a}, NR⁷R⁸, C₁₋₄alkyleneNR⁷R⁸ and C₁₋₄alkyl. In an embodiment, R³ is selected from H, F, Cl and C₁₋₄alkyl. In an embodiment, R³ is selected from H, F, Cl, CH₃, CH₂CH₃, CF₂H, CF₃, CFH₂, CH₂CF₂H, and CH₂CF₃. In an embodiment, R³ is selected from H, F, CF₃, CF₂H, CH₂CF₂H and CH₃. In an embodiment, R³ is selected from H, F, CF₃, CF₂H and CH₂CF₂H. In an embodiment, R³ is selected from H, F, CF₃, and CH₃. In an embodiment, R³ is selected from H and F. In an embodiment, R³ is F. In an embodiment, R³ is H.

In an embodiment, R³ is OR^{5a}. In an embodiment, R^{5a} is selected from H and C₁₋₄alkyl. In an embodiment, R^{5a} is selected from H, CH₃, CH₂CH₃, CF₃, CFH₂, CF₂H, CH₂CF₂H, and CH₂CF₂H. In an embodiment, R^{5a} is selected from H, CH₃, CH₂CH₃, CF₃, CFH₂, CF₂H, CH₂CF₂H, and CH₂CF₂H. In an embodiment, R^{5a} is selected from H, CH₃, CF₃ and CF₂H. In an embodiment, R^{5a} is CF₂H. Accordingly, in an embodiment, R³ is selected from OH, OCH₃, OCH₂CH₃, OCF₃, OCFH₂, OCHF₂, OCH₂CF₂H, and OCH₂CF₂H.

In an embodiment, R³ is selected from NR⁷R⁸ and C₁₋₄alkyleneNR⁷R⁸. In an embodiment, R³ is selected from NR⁷R⁸ and C₁₋₂alkyleneNR⁷R⁸.

In an embodiment, R⁷ and R⁸ are each independently selected from H and C₁₋₄alkyl. In an embodiment, R⁷ and R⁸ are each independently selected from H, CH₃ and CF₃ In an embodiment, one of R⁷ and R⁸ is H and the other is CH₃. In an embodiment, R⁷ and R⁸ are both CH₃. In an embodiment, R⁷ and R⁸ are both H.

In an embodiment, R⁷ and R⁸ are joined to form, together with the nitrogen atom therebetween, a 3 to 7-membered saturated or unsaturated ring optionally containing one additional heteromoiety selected from N, NH, NC₁₋₆alkyl, O, S, S(O), and SO₂, and optionally substituted with one or more of halo and C₁₋₆alkyl. In an embodiment, R⁷ and R⁸ are joined to form, together with the nitrogen atom therebetween to form a 3- to 7-membered heterocyclic ring selected from azetidinyl, diazetidinyl, pyrrolidinyl, imidazolidinyl, pyrazolidinyl, thiazolidinyl, isothiozolidinyl, piperidinyl, diazinanyl (e.g. piperazinyl), morpholinyl and azepanyl, and optionally substituted with one to four of halo and C₁₋₆alkyl. In an embodiment, R⁷ and R⁸ are joined to form, together with the nitrogen atom therebetween, a 4- to 6-saturated ring, and optionally substituted with one to four of halo and C₁₋₄alkyl. In an embodiment R⁷ and R⁸ are joined to form, together with the nitrogen atom therebetween, aziridinyl, azetidinyl, pyrrolidinyl, or piperidinyl, and optionally substituted with one to three of Cl, F and C₁₋₄alkyl.

In an embodiment, one of X² and X³ is N and the other is CR⁴. In an embodiment, X² is N and X³ is CR⁴. In an embodiment, X² is CR⁴ and X³ is N. In an embodiment, both X² and X³are, independently, CR⁴. In an embodiment, both X² and X³are N.

In an embodiment, X¹ is N, and X² and X³are both, independently, CR⁴. In an embodiment, X¹ is CR³, and one of X² and X³ is N and the other is CR⁴. In an embodiment, X¹ is CR³, X² is CR⁴ and X³ is N. In an embodiment, X¹ is CR³ and X² is N and X³ is CR⁴. In an embodiment, X¹ is CR³ and X² and X³ are both, independently, CR⁴. In an embodiment, X¹ is CR³ and both X² and X³ are N.

In an embodiment, each R⁴ is independently selected from H, halo, and C₁₋₄alkyl. In an embodiment, each R⁴ is independently selected from H, F, Cl, and C₁₋₄alkyl. In an embodiment, each R⁴is independently selected from H, F, Cl, CH₃, CF₃, CH₂F, and CHF₂. In an embodiment, each R⁴ is independently selected from H, F, Cl, CH₃, and CF₃. In an embodiment, each R⁴ is independently selected from H and F.

In an embodiment, one of X² and X³ is N and the other is CH. In an embodiment, X² is N and X³is CH. In an embodiment, X² is selected from CH, CF, CCl, CCH₃, and CCF₃ and X³ is CH. In an embodiment, X² is CF or CCl and X³is CH. In an embodiment, X² is CH and X³ is selected from CH, CF, CCl, CCH₃, and CCF₃. In an embodiment, one of X² and X³ is CF and the other is CH. In an embodiment, X² is CF and X³ is CH. In an embodiment, X² and X³ are both CF. In an embodiment, X² and X³ are both CH. In an embodiment, X² and X³are both N.

In an embodiment, Q is C₁₋₃alkylene optionally interrupted by a heteromoiety selected from O, S, S(O), SO₂, and NR⁵ and/or optionally substituted with one or more of R⁶.

In an embodiment, Q is C₁₋₃alkylene optionally interrupted by a heteromoiety selected from O, S, S(O), SO₂, and NR⁵. In an embodiment, Q is C₁₋₃alkylene optionally interrupted by a heteromoiety selected from O, SO₂, and NR⁵. In an embodiment, Q is C₁₋₃alkylene optionally interrupted by a heteromoiety selected from O and NR⁵. In an embodiment, Q is C₁₋₃alkylene optionally interrupted by a heteromoiety selected from O and SO₂. In an embodiment, Q is C₁₋₃alkylene optionally interrupted by O. In an embodiment, Q is C₁₋₂alkylene optionally interrupted by SO₂.

In an embodiment, R⁵ is selected from H and C₁₋₄alkyl. In an embodiment, R⁵ is selected from CH₃, CH₂CH₃, CH(CH₃)₂, C(CH₃)₄ and CH₂CH(CH₃)₂. In an embodiment, R⁵ is selected from CH₃, CH₂CH₃, CF₂H, CF₃, CFH₂, CH₂CF₂H, and CH₂CF₃. In an embodiment, R⁵ is selected from CF₂H, CH₃ and CF₃

In an embodiment, R⁵ and R^{5a} are independently selected from H and C. ₄alkyl. In an embodiment, R⁵ and R^{5a} are independently selected from CH₃, CH₂CH₃, CH(CH₃)₂, C(CH₃)₄ and CH₂CH(CH₃)₂. In an embodiment, R⁵ and R^{5a} are independently selected from CH₃, CH₂CH₃, CF₂H, CF₃, CFH₂, CH₂CF₂H, and CH₂CF₃. In an embodiment, R⁵ and R^{5a} are independently selected from CH₃ and CF₃

In an embodiment, Q is C₁₋₃alkylene and optionally substituted with one to three of R⁶. In an embodiment, Q is CH₂ or CH₂CH₂ and optionally substituted with one or two of R⁶. In an embodiment, Q is CH₂. In an embodiment, Q is CH₂CH₂.

In an embodiment, each R⁶ is independently selected from ═O, F, Cl, C₁₋₄alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR^{7a}R^{8a}, and C₁₋₆alkyleneNR^{7a}R^{8a}. In an embodiment, each R⁶ is independently selected from ═O, F, Cl, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC. ₄alkyl, NR^{7a}R^{8a}, and C₁₋₄alkyleneNR^{7a}R^{8a}. In an embodiment, one of R⁶ is ═O. In an embodiment, Q is substituted with one R⁶ and R⁶ is ═O.

In an embodiment, each R⁶ is independently selected from F, C, OH, C₁₋₄alkyl OC₁₋₄alkyl and NR^{7a}R^{8a}. In an embodiment, each R⁶ is independently selected from F, C, OH, CH₃, CH₂CH₃, CF₂H, CF₃, CFH₂, CH₂CF₂H, OCH₃, OCH₂CH₃, OCF₃, OCF₂H, OCH(CH₃)₂ and NR^{7a}R^{8a}. In an embodiment, each R⁶ is independently selected from F, C, OH, CH₃, CF₂H, CF₃, CFH₂, OCH₃, OCF₃, OCF₂H and NR^{7a}R^{8a}. In an embodiment, one to three of R⁶ are independently selected from F, C, CH₃, CF₂H, CF₃, OCH₃, OCF₃, OCF₂H and NR^{7a}R^{8a}. In an embodiment, one to three of R⁶ are independently selected from F, C, CH₃, CF₂H, CF₃, OCH₃, OCF₃ and OCF₂H. In an embodiment, one to four of R⁶ are independently selected from F, CH₃, and OCH₃.

In an embodiment, each R⁶ is independently selected from F, C and C₁₋₄alkyl. In an embodiment, each R⁶ is independently selected from F, C, CH₃, CH₂CH₃, CF₂H, CF₃, CFH₂, CH₂CF₂H, and CH₂CFH₂. In an embodiment, each R⁶ is independently selected from F, C, CH₃, CF₂H, CF₃ and CH₂CF₂H. In an embodiment, each R⁶ is independently selected from F, Cl, CH₃, and CF₃. In an embodiment, each R⁶ is independently selected from F, CH₃, and CF₃. In an embodiment, each R⁶ is independently selected from F, CH₃, and CF₃. In an embodiment, each R⁶ is independently selected from F and CH₃. In an embodiment, each R⁶ is independently F. In an embodiment, each R⁶ is independently CH₃. In an embodiment, one or two of R⁶ is F. In an embodiment, one or two of R⁶ is CH₃.

In an embodiment, each R⁶ is independently selected from C₃₋₆cycloalkyl, C₃₋₆heterocycloalkyl, C₁₋₄alkyleneC₃₋₆cycloalkyl and C₁₋₄alkyleneC₃₋₆heterocycloalkyl. In an embodiment, each R⁶ is independently selected from C₃₋₆cycloalkyl, C₃₋₆heterocycloalkyl, C₁₋₂alkyleneC₃₋₆cycloalkyl and C₁₋₂alkyleneC₃₋₆heterocycloalkyl. In an embodiment, the cycloalkyl in R⁶ is selected from cyclopropyl, cyclobutyl, cyclopentyl and cyclohexyl. In an embodiment, the cycloalkyl in R⁶ is selected from cyclopropyl and cyclobutyl.

In an embodiment, the heterocycloalkyl R⁶ is selected from aziridinyl, oxiranyl, thiiranyl, oxaxiridinyl, dioxiranyl, azetidinyl, oxetanyl, thietanyl, diazetidinyl, dioxetanyl, dithietanyl, tetrahydrofuranyl, tetrahydrothiophenyl, pyrrolidinyl, imidazolidinyl, pyrazolidinyl, isoxthiolidinyl, thiazolidinyl, isothiazolidinyl, dioxolanyl, dithiolanyl, piperidinyl, triazolyl, furazanyl, oxadiazolyl, thiadiazolyl, dioxazolyl, dithiazolyl, tetrazolyl, oxatetrazolyl, tetrahydropyranyl, diazinanyl (e.g., piperazinyl), morpholinyl, thiomorpholinyl, dioxanyl and dithianyl. In an embodiment, the heterocycloalkyl in R⁶ is selected from azetidinyl, oxetanyl, thietanyl, tetrahydrofuranyl, pyrrolidinyl, imidazolidiny and pyrazolidinyl.

In an embodiment, each R⁶ is independently selected from OH and OC₁₋₄alkyl. In an embodiment, each R⁶ is independently selected from OH and OC₁₋₄alkyl. In an embodiment, each R⁶ is independently selected from OH, OCH₃, OCH₂CH₃, OCF₃, OCF₂H, and OCH(CH₃)₂. In an embodiment, one or two of R⁶ are independently selected from OH, OCH₃, OCH₂CH₃, OCF₃, OCF₂H, and OCH(CH₃)₂. In an embodiment, one R⁶ is selected from OH, OCH₃, OCF₃, and OCF₂H.

In an embodiment, one of R⁶ is NR^{7a}R^{8a} or C₁₋₄alkyleneNR^{7a}R^{8a}. In an embodiment, one of R⁶ is NR^{7a}R^{8a} or C₁₋₂alkyleneNR^{7a}R^{8a}. In an embodiment, one of R⁶ is NR^{7a}R^{8a}. In an embodiment, one of R⁶ is C₁₋₄alkyleneNR^{7a}R^{8a}. In an embodiment, the R^{7a} and R^{8a} in NR^{7a}R^{8a} or C₁₋₂alkylene NR^{7a}R^{8a} of R⁶ are both CH₃ or are both H. In an embodiment, one R⁴ is NR^{7a}R^{8a} and R^{7a} and R^{8a} in R⁶ are both H.

In an embodiment, R^{7a} and R^{8a} in R⁶ are each independently selected from H and C₁₋₄alkyl. In an embodiment, R^{7a} and R^{8a} in R⁶ are each independently selected from H, CH₃, and CF₃ In an embodiment, one of R^{7a} and R^{8a} in R⁶ is H and the other is CH₃. In an embodiment, R^{7a} and R^{8a} in R⁶ are both CH₃. In an embodiment, one R⁶ is NR^{7a}R^{8a} and R^{7a} and R^{8a}in R⁶ are both CH₃ or both H. In an embodiment, one R⁶ is NR^{7a}R^{8a} and R^{7a} and R^{8a} in R⁶ are both H.

In an embodiment, R^{7a} and R^{8a} are joined to form, together with the nitrogen atom therebetween, a 3- to 7-saturated or unsaturated ring optionally containing one additional heteromoiety selected from N, NH, NC₁₋₆alkyl, O, S, S(O), and SO₂, and optionally substituted with one or more of halo and C₁₋₆alkyl. In an embodiment, R^{7a} and R^{8a} are taken together with the nitrogen atom therebetween to form a 3- to 7-membered heterocyclic ring selected from azetidinyl, diazetidinyl, pyrrolidinyl, imidazolidinyl, pyrazolidinyl, thiazolidinyl, isothiozolidinyl, piperidinyl, diazinanyl (e.g. piperazinyl), morpholinyl and azepanyl, optionally substituted with one to four of halo and C₁₋₆alkyl. In an embodiment, R^{7a} and R^{8a}are joined to form, together with the nitrogen atom therebetween, a 4- to 6-heterocyclic ring, optionally substituted with one to four of halo and C₁₋₄alkyl. In an embodiment R^{7a} and R^{8a} are joined to form, together with the nitrogen atom therebetween, aziridinyl, azetidinyl, pyrrolidinyl, or piperidinyl, optionally substituted with one to three of Cl, F and C₁₋₄alkylatom.

In an embodiment, Q is unsubstituted. In an embodiment, Q is substituted with one or two of R⁶. In an embodiment, Q is substituted with one or two of R⁶, and R⁶ is C₁₋₄alkyl. In an embodiment, Q is substituted with one or two of R⁶, and R⁶ is CH₃. In an embodiment, Q is C₁₋₃alkylene and is unsubstituted. In an embodiment, Q is C₁₋₃alkylene and is substituted with one or two of R⁶. In an embodiment, Q is C₁₋₃alkylene and is substituted with one or two of R⁶, and one of R⁶ is C₁₋₄alkyl. In an embodiment, Q is C₁₋₃alkylene and is substituted with one or two of R⁶, and one of R⁶ is CH₃ln an embodiment, Q is C₁₋₃alkylene and is substituted with one to four of R⁶, and each R⁶ is independently selected from F, Cl, CH₃, CF₂H, CF₃, OCH₃, OCF₃ and OCF₂H. In an embodiment, Q is C₁₋₃alkylene and is substituted with one to four of R⁶, and each R⁶ is independently selected from F, CH₃ and OCH₃.

In an embodiment, Q is C₁₋₃alkylene and disubstituted on one carbon atom with R^{6c} and R^{6b}. In an embodiment, Q is C₂alkylene or C₂alkylene, and disubstituted on one carbon atom with R^{6a} and R^{6b}. In an embodiment, Q is CR^{6a}R^{6b}.

In an embodiment, R^{6a} and R^{6b} are joined to form, together with the carbon atom therebetween, a 3- to 6-membered saturated or unsaturated ring optionally containing one heteromoiety selected from N, NH, NC₁₋₆alkyl, O, S, S(O), and SO₂ and optionally substituted with one or more, or one to four, of halo and C₁₋₄alkyl.

In an embodiment, R^{6a} and R^{6b} are joined to form, together with the carbon atom therebetween, a 3- to 6-membered cycloalkyl ring and optionally substituted with one or more of halo and C₁₋₄alkyl. In an embodiment, R^{6a} and R^{6b} are joined to form, together with the carbon atom therebetween, a 3- to 6-membered cycloalkyl selected from cyclopropyl, cyclobutyl, cyclopentyl and cyclohexyl and optionally substituted with one or more of halo and C₁₋₄alkyl. In an embodiment, R^{6a} and R^{6b} are joined to form, together with the atom therebetween, a 3- to 5-membered cycloalkyl ring, and optionally substituted with one to four of halo and C₁₋₄alkyl. In an embodiment, R^{6a} and R^{6b} are joined to form, together with the carbon atom therebetween, a cyclopropyl, a cyclobutyl, or a cyclopentyl ring, and optionally substituted with one to three of halo and C₁₋₄alkyl. In an embodiment, R^{6a} and R^{6b} are joined to form, together with the carbon atom therebetween, a cyclopropyl or a cyclobutyl ring, and optionally substituted with one or two of halo and C₁₋₄alkyl. In an embodiment, R^{6a} and R^{6b}are joined to form, together with the carbon atom therebetween, a cyclopropyl or a cyclobutyl ring. In an embodiment, R^{6a} and R^{6b} are joined to form, together with the carbo atom therebetween, a cyclopropyl ring and optionally substituted with one or two of Cl, F and C₁₋₄alkyl. In an embodiment, R^{6a} and R^{6b} are joined to form, together with the carbon atom therebetween, a cyclopropyl or a cyclobutyl ring. In an embodiment, R^{6a} and R^{6b} are joined to form, together with the carbon atom therebetween, an unsubstituted cyclopropyl ring.

In an embodiment, R^{6a} and R^{6b} are joined to form, together with the carbon atom therebetween, a 3- to 6-membered heterocycloalkyl ring, and optionally substituted with one or more of halo and C₁₋₄alkyl. In an embodiment, R^{6a} and R^{6b} are joined to form, together with the carbon atom therebetween, a 3- to 6-membered heterocycloalkyl ring selected from aziridinyl, oxiranyl, thiiranyl, oxaxiridinyl, dioxiranyl, azetidinyl, oxetanyl, thietanyl, diazetidinyl, dioxetanyl, dithietanyl, tetrahydrofuranyl, tetrahydrothiophenyl, pyrrolidinyl, imidazolidinyl, pyrazolidinyl, isoxthiolidinyl, thiazolidinyl, isothiazolidinyl, dioxolanyl, dithiolanyl, piperidinyl, triazolyl, furazanyl, oxadiazolyl, thiadiazolyl, dioxazolyl, dithiazolyl, tetrazolyl, oxatetrazolyl, tetrahydropyranyl, diazinanyl (e.g., piperazinyl), morpholinyl, thiomorpholinyl, dioxanyl, and dithianyl. In an embodiment, R^{6a} and R^{6b} are joined to form, together with the carbon atom therebetween, a 3- to 5-membered heterocycloalkyl ring. In an embodiment, R^{6a} and R^{6b} are joined to form, together with the carbon atom therebetween, a 3- to 5-membered heterocycloalkyl ring selected from oxiranyl, oxetanyl, azetidinyl, thietanyl, pyrrolidinyl, tetrahydrofuranyl, and tetrahydrothiophenyl ring. In an embodiment, R^{6a} and R^{6b} are joined to form, together with the carbon atom therebetween, oxetanyl or azetidinyl. In an embodiment, R^{6a} and R^{6b} are joined to form, together with the carbon atom therebetween, oxetanyl. In an embodiment, R^{6a} and R^{6b} are joined to form, together with the atom therebetween to form [symbol] herein “·” indicates a point of attachment to Q.

In an embodiment, Q is C₂₋₄alkenylene optionally substituted with one or two of R^{6c}. In an embodiment, Q is C═C optionally substituted with one or two of R^{6c}.

In an embodiment, Q is selected from C═N and N═C and is optionally substituted with R^{6c}. In an embodiment, Q is C═N or N═C.

In an embodiment, each R^{6c}is independently selected from F, Cl, C₁₋₄alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR^{7a}R^{8a}, and C₁₋₆alkyleneNR^{7a}R^{8a}. In an embodiment, each R^{6c} is independently selected from F, Cl, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC₁₋₄alkyl, NR^{7a}R^{8a}, and C. ₄alkyleneNR^{7a}R^{8a}.

In an embodiment, each R^{6c} is independently selected from F, Cl, OH, C. 4alkyl, OC₁₋₄alkyl, C₁₋₂alkyleneNR^{7a}R^{8a} and NR^{7a}R^{8a}. In an embodiment, each R^{6c} is independently selected from F, Cl, OH, CH₃, CH₂CH₃, CF₂H, CF₃, CFH₂, CH₂CF₂H, OCH₃, OCH₂CH₃, OCF₃, OCF₂H, OCH(CH₃)₂, C₁₋₂alkyleneNR^{7a}R^{8a} and NR^{7a}R^{8a}. In an embodiment, each R^{6c} is independently selected from F, Cl, OH, CH₃, CF₂H, CF₃, CFH₂, OCH₃, OCF₃, OCF₂H and NR^{7a}R^{8a}. In an embodiment, each R^{6c} are independently selected from F, Cl, CH₃, CF₂H, CF₃, OCH₃, OCF₃, OCF₂H, C₁₋₂alkyleneNR^{7a}R^{8a} and NR^{7a}R^{8a}. In an embodiment, each R^{6c} are independently selected from F, Cl, CH₃, CF₂H, CF₃, OCH₃, OCF₃, OCF₂H and NR^{7a}R^{8a}.

In an embodiment, each R^{6c}is independently selected from F and C₁₋₄alkyl. In an embodiment, each R^{6c} is independently selected from F, C, CH₃, CH₂CH₃, CF₂H, CF₃, CFH₂, CH₂CF₂H, and CH₂CH₂F. In an embodiment, each R^{6c} is independently selected from F, C, CH₃, CF₂H, CF₃ and CH₂CF₂H. In an embodiment, each R^{6c} is independently selected from F, C, CH₃, and CF₃. In an embodiment, each R^{6c} is independently selected from F, CH₃, and CF₃. In an embodiment, each R^{6c} is independently selected from F and CH₃. In an embodiment, each R^{6c} is independently F. In an embodiment, each R^{6c} is independently CH₃. In an embodiment, each R^{6c} is independently F. In an embodiment, each R^{6c} is independently CH₃. In an embodiment, one or two R^{6c} is F. In an embodiment, one or two R^{6c} is CH₃. In an embodiment, one or two R^{6c} is F. In an embodiment, each R^{6c} is CH₃.

In an embodiment, each R^{6c}is independently selected from C₃₋₆cycloalkyl, C₃₋₆heterocycloalkyl, C₁₋₄alkyleneC₃₋₆cycloalkyl and C₁₋₄alkyleneC₃₋₆heterocycloalkyl. In an embodiment, each R^{6c} is independently selected from C₃₋₆cycloalkyl, C₃₋₆heterocycloalkyl, C₁₋₂alkyleneC₃₋₆cycloalkyl and C₁₋₂alkyleneC₃₋₆heterocycloalkyl. In an embodiment, the cycloalkyl in R^{6c} is selected from cyclopropyl, cyclobutyl, cyclopentyl and cyclohexyl. In an embodiment, the cycloalkyl in R^{6c} is selected from cyclopropyl and cyclobutyl.

In an embodiment, the heterocycloalkyl R^{6c} is selected from aziridinyl, oxiranyl, thiiranyl, oxaxiridinyl, dioxiranyl, azetidinyl, oxetanyl, thietanyl, diazetidinyl, dioxetanyl, dithietanyl, tetrahydrofuranyl, tetrahydrothiophenyl, pyrrolidinyl, imidazolidinyl, pyrazolidinyl, isoxthiolidinyl, thiazolidinyl, isothiazolidinyl, dioxolanyl, dithiolanyl, piperidinyl, triazolyl, furazanyl, oxadiazolyl, thiadiazolyl, dioxazolyl, dithiazolyl, tetrazolyl, oxatetrazolyl, tetrahydropyranyl, diazinanyl (e.g., piperazinyl), morpholinyl, thiomorpholinyl, dioxanyl, and dithianyl. In an embodiment, the heterocycloalkyl in R^{6c} is selected from azetidinyl, oxetanyl, thietanyl, tetrahydrofuranyl, pyrrolidinyl, imidazolidiny, and pyrazolidinyl.

In an embodiment, each R^{6c} is independently selected from OH and OC. ₄alkyl. In an embodiment, each R^{6c} is independently selected from OH and OC₁₋₄alkyl. In an embodiment, each R⁶ is independently selected from OH, OCH₃, OCF₃, OCF₂H, OCH₂CH₃ and OCH(CH₃)₂. In an embodiment, one R^{6c} is selected from OH, OCH₃, OCF₃, and OCF₂H.

In an embodiment, one of R^{6c} is NR^{7a}R^{8a} or C₁₋₄alkyleneNR^{7a}R^{8a}. In an embodiment, one of R^{6c} is NR^{7a}R^{8a} or C₁₋₂alkyleneNR^{7a}R^{8a}. In an embodiment, one of R^{6c} is NR^{7a}R^{8a}. In an embodiment, R^{6c} is NR^{7a}R^{8a} and R^{7a} and R^{8a} in R^{6c} are both CH₃ or both H. In an embodiment, one R^{6c} is NR^{7a}R^{8a} and R^{7a} and R^{8a} in R^{6c} are both H.

In an embodiment, R^{7a} and R⁸a in R^{6c}are each independently selected from H and C₁₋₄alkyl. In an embodiment, R^{7a} and R^{8a} in R^{6c} are each independently selected from H, CH₃, and CF₃ In an embodiment, one of R^{7a} and R^{8a} in R^{6c}is H and the other is CH₃. In an embodiment, R^{7a} and R^{8a} in R^{6c} are both CH₃.

In an embodiment, R^{7a} and R^{8a} in R^{6c} are joined to form, together with the nitrogen atom therebetween, a 3- to 7-membered saturated or unsaturated ring optionally containing one additional heteromoiety selected from N, NH, NC₁₋₆alkyl, O, S, S(O), and SO₂, and optionally substituted with one or more of halo and C₁₋₆alkyl. In an embodiment, R^{7a} and R^{8a} in R^{6c} are taken together with the nitrogen atom therebetween to form a 3- to 7-membered heterocyclic ring selected from azetidinyl, diazetidinyl, pyrrolidinyl, imidazolidinyl, pyrazolidinyl, thiazolidinyl, isothiozolidinyl, piperidinyl, diazinanyl (e.g. piperazinyl), morpholinyl and azepanyl, optionally substituted with one to four of halo and C₁₋₆alkyl. In an embodiment, R^{7a} and R^{8a} in R^{6c} are joined to form, together with the nitrogen atom therebetween, a 4- to 6-membered heterocyclic ring, optionally substituted with one to four of halo and C₁₋₄alkyl. In an embodiment R^{7a} and R^{8a} in R^{6c} are joined to form, together with the nitrogen atom therebetween, aziridinyl, azetidinyl, pyrrolidinyl, or piperidinyl, optionally substituted with one to three of F, Cl and C₁₋₄alkyl.

In an embodiment, Q is C₁₋₃alkylene optionally interrupted by NR⁵. In an embodiment, R⁵is selected from H and C₁₋₄alkyl. In an embodiment, R⁵ is selected from H, CH₃, CH₂CH₃, CF₂H, CF₃, CFH₂, CH₂CF₂H and CH₂CF₃.

In an embodiment, Q is C₁₋₃alkylene optionally interrupted by a heteromoiety selected from O, S, S(O), SO₂, and NR⁵. In an embodiment, Q is C₁₋₃alkylene optionally interrupted by a heteromoiety selected from O, SO₂, and NR⁵. In an embodiment, Q is C₁₋₃alkylene optionally interrupted by a heteromoiety selected from O and NR⁵. In an embodiment, Q is C₁₋₃alkylene optionally interrupted by a heteromoiety selected from O and SO₂. In an embodiment, Q is C₁₋₃alkylene optionally interrupted by O. In an embodiment, Q is C₂alkylene optionally interrupted by O. In an embodiment, Q is C₁₋₂alkylene optionally interrupted by SO₂.

In an embodiment, R⁵is selected from H and C₁₋₄alkyl. In an embodiment, R⁵ is selected from H, CH₃, CH₂CH₃, CF₂H, CF₃, CFH₂, CH₂CF₂H, and CH₂CF₃.

In an embodiment, Q is C₁alkylene or C₂alkylene, and is disubstituted on one carbon atom with R^{6a} and R^{6b} and R^{6a} and R^{6b} are joined to form, together with the carbon atom therebetween, a 3- to 5-membered cycloalkyl ring, and optionally substituted with one or more of halo and C₁₋₄alkyl. In an embodiment, Q is CR^{6a}R^{6b}. In R^{6a} and R^{6b} are joined to form, together with the carbon atom therebetween, a cyclopropyl or a cyclobutyl ring, and optionally substituted with one or more of halo and C₁₋₄alkyl.

In an embodiment, Q is C₂₋₄alkenylene optionally substituted with one or two of R^{6c} , and each R^{6c} is independently selected from F, Cl, C₁₋₄alkyl and OC₁₋₄alkyl. In an embodiment, Q is C═C optionally substituted with one or two of R^{6c} , and each R^{6c} is independently selected from F, Cl, C₁₋₄alkyl and OC₁₋₄alkyl. In an embodiment, Q is C═C optionally substituted with one or two of R^{6c} , and each R^{6c} is independently selected from F, Cl, CH₃ and OCH₃OCF₃ and OCF₂H. In an embodiment, Q is C═C optionally substituted with one or two of R^{6c} , and each R^{6c} is independently selected from F and C₁₋₄alkyl. In an embodiment, R^{6c} is selected from F and CH₃. In an embodiment, Q is C═C substituted with F. In an embodiment, Q is C═C substituted with C. In an embodiment, Q is C═C substituted with OCH₃. In an embodiment, Q is C═C substituted with CH₃. In an embodiment, Q is C═C substituted with F and CH₃.

In an embodiment, Q is selected from C═N and N═C and is optionally substituted with one or two of R^{6c} , and each R^{6c} is independently selected from F, Cl, C. ₄alkyl, OC₁₋₄alkyl, NR^{7a}R^{8a}, cyclopropyl and cyclobutyl. In an embodiment, Q is selected from C═N and N═C and is optionally substituted with one or two of R^{6c} , and each R^{6c} is independently selected from F, Cl, CH₃, OCH₃, OCF₃ OCF₂H, NR^{7a}R^{8a}, cyclopropyl and cyclobutyl. In an embodiment, Q is selected from C═N and N═C and is optionally substituted with one or two R^{6c} , and each R^{6c} is independently selected from F, Cl, CH₃, OCH₃, OCF₃ and OCF₂H. In an embodiment, Q is selected from C═N and N═C and is optionally substituted with one or two R^{6c} , and each R^{6c} is independently selected from F and CH₃. In an embodiment, Q is selected from C═N or N═C and is optionally substituted with one or two R^{6c} , and each R^{6c} is independently selected from cyclopropyl and cyclobutyl. In an embodiment, Q is selected from C═N or N═C and is optionally substituted with one or two R^{6c} , and each R^{6c} is independently selected from F and C₁₋₄alkyl. In an embodiment, Q is selected from C═N and N═C is optionally substituted with R^{6c} , and R^{6c} is selected from F and CH₃. In an embodiment, Q is selected from C═N or N═C and is optionally substituted with R^{6c} , and R^{6c} is selected from cyclopropyl and cyclobutyl. In an embodiment, Q is selected from C═N or N═C and is optionally substituted with R^{6c} and R^{6c} is selected from F and C₁₋₄alkyl. In an embodiment, Q is selected from C═N or N═C and is optionally substituted with one or two R^{6c} , and one R^{6c} is NR^{7a}R^{8a}. In an embodiment, Q is selected from C═N or N═C and is optionally substituted with one or two R^{6c} , and one R^{6c} is C₁₋₂alkyleneNR^{7a}R^{8a}.

In an embodiment, R¹ and R² are independently selected from H, F and Cl. In an embodiment, one of R¹ and R² is F or Cl and the other is H. In an embodiment, one of R¹ and R² is F and the other is H. In an embodiment, R¹ is F and R² is H. In an embodiment, R¹ is H and R² is F.

In an embodiment, Cy¹ is C₆₋₁₆aryl or C₅₋₁₇heteroaryl, and Cy¹ is unsubstituted. In an embodiment, Cy¹ is C₆₋₁₆aryl or C₅₋₁₇heteroaryl, Cy¹ is substituted with one to three of R⁹. In an embodiment, Cy¹ is C₆₋₁₆aryl or C₅₋₁₇heteroaryl, Cy¹ is substituted with Z-Cy². In an embodiment, Cy¹ is C₆₋₁₆aryl or C₅₋₁₇heteroaryl, Cy¹ is substituted with Z-Cy² and one to three of R⁹.

In an embodiment, Cy¹ is C₆₋₁₀aryl. In an embodiment, Cy¹ is phenyl, indanyl, or naphthyl. In an embodiment, Cy¹ is phenyl.

In an embodiment, Cy¹ is a bicyclic C₉₋₁₁aryl wherein the aryl group is fused to a heterocycloalkyl group, and Cy¹ is unsubstituted or is substituted with one or more of R⁹ or is substituted with Z-Cy², or is substituted with Z-Cy² and one or more of R⁹ . In an embodiment, Cy¹ is benzo-fused to a C₉₋₁₁heterocycloalkyl. In an embodiment, Cy¹ is benzo-fused to a C₄₋₈heterocycloalkyl. In an embodiment, Cy¹ is selected from indolinyl, dihydrobenzofuranyl, dihydrobenzothiophenyl, benzofuranonyl, 1,1-dioxido-dihydrobenzothiophenyl, benzodioxolyl, benzodioxanyl, tetrahydroisoquinolinyl, tetrahydroquinolinyl, 1-oxo-tetrahydroisoquinolinyl, 1-oxo-tetrahydroquinolinyl, dihydrobenzodioxepinyl, benzoxazinyl, isochromanyl, chromanyl, chromanonyl, isochromenyl, chromenyl, and chromenonyl. In an embodiment, Cy¹ is selected from

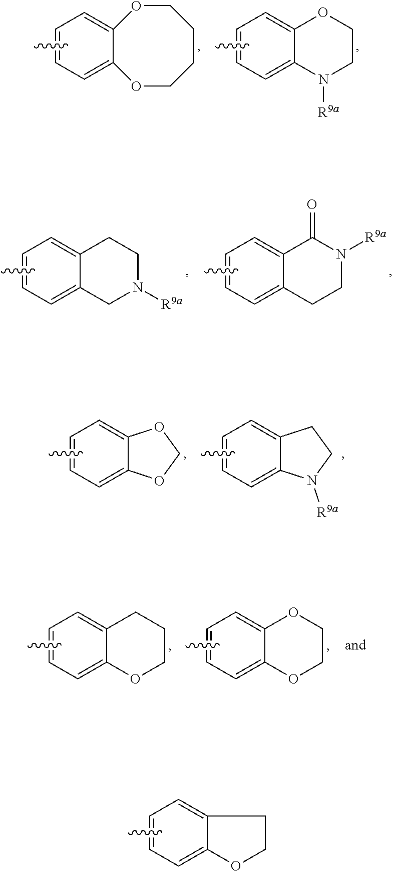

wherein [symbol] represents the point of attachment of Cy¹ to the remainder of molecule and the Cy¹ is unsubstituted or is substituted with one or more of R⁹, and wherein R^{9a} is selected from H, R⁹, and Z-Cy².

In an embodiment, Cy¹ is a tricyclic C₁₁₋₁₇aryl, wherein the aryl group is fused and/or spirofused to one or two heterocycloalkyl groups and Cy¹ is unsubstituted or is substituted with one or more of R⁹ or is substituted with Z-Cy², or is substituted with Z-Cy² and one or more of R⁹. In an embodiment, Cy¹ is a benzofused spirofused tricyclic C₁₁₋₁₇aryl. In an embodiment, Cy¹ is selected from spirobenzodioxinepiperidinyl, oxospirochromanepiperidinyl, and spirochromanepiperidinyl. In an embodiment, Cy¹ is selected from

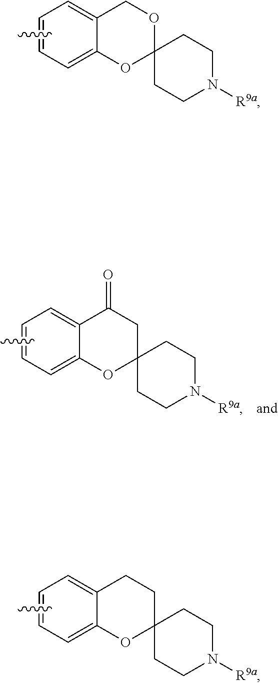

wherein [symbol] represents the point of attachment of Cy¹ to the remainder of molecule and the Cy¹ is unsubstituted or is substituted with one or more of R⁹, and wherein R^{9a}is selected from H, R⁹, and Z-Cy².

In an embodiment, Cy¹ is C₅₋₆heteroaryl, and Cy¹ is unsubstituted or is substituted with one or more of R⁹ or is substituted with Z-Cy², or is substituted with Z-Cy² and one or more of R⁹. In an embodiment, Cy¹ is selected from pyrrolyl, imidazolyl, oxazolyl, pyrazolyl, thiazolyl, pyridinyl, pyrazinyl, pyridazinyl, and pyrimidinyl. In an embodiment, Cy¹ is selected from pyrrolyl, pyrazolyl, thiazolyl, pyridinyl, pyrazinyl, and pyrimidinyl. In an embodiment, Cy¹ is selected from pyrazolyl, pyridinyl, pyrazinyl, and pyrimidinyl. In an embodiment, Cy¹ is pyridinyl. In an embodiment, Cy¹ is pyrazolyl.

In an embodiment, Cy¹ is bicyclic C₉₋₁₁heteroaryl, and Cy¹ is unsubstituted or is substituted with one or more of R⁹or is substituted with Z-Cy², or is substituted with Z-Cy² and one or more of R⁹ In an embodiment, Cy¹ is selected from benzofuranyl, benzothiophenyl, benzodioxolyl, quinolinyl, and isoquinolyl.

In an embodiment, Cy¹ is a pyrazolodiazepinonyl. In an embodiment, Cy¹ is

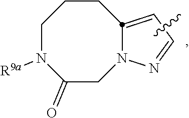

wherein [symbol] represents the point of attachment of Cy¹ to the remainder of molecule and the Cy¹ is unsubstituted or is substituted with one or more of R⁹, and wherein R^{9a}is selected from H, R⁹, and Z-Cy².

In an embodiment, Cy¹ is unsubstituted or substituted with one to four of R⁹. In an embodiment, Cy¹ is unsubstituted or substituted with one to three of R⁹. In an embodiment, Cy¹ is unsubstituted or substituted with one to two of R⁹. In an embodiment, Cy¹ is unsubstituted or substituted with one R⁹. In an embodiment, Cy¹ is unsubstituted or substituted with Z-Cy² and one to four of R⁹. In an embodiment, Cy¹ is unsubstituted or substituted with Z-Cy² and one to three of R⁹. In an embodiment, Cy¹ is unsubstituted or substituted with Z-Cy² and one to two of R⁹. In an embodiment, Cy¹ is unsubstituted or substituted with one of Z-Cy² and R⁹.

In an embodiment, each R⁹is independently selected from halo, ═O, CN, NO₂, C₁₋₆alkyl, C₂₋₆alkenyl, C₂₋₆alkynyl, OR¹⁰, C(O)R¹⁰, CO₂R¹⁰, P(O)R¹⁰R¹¹, P(O)(OR¹⁰)(OR¹¹), SR¹⁰, S(O)R¹⁰, SO₂R¹⁰, S(O)(═NR¹¹)R¹⁰, SO₂NR¹⁰R¹¹, SiR¹²R^{12a}R^{12a}, C₁₋₆alkyleneOR¹⁰, OC₁₋₆alkyleneOR¹⁰, C₁₋₆alkyleneNR^{10a}R^{11a} OC₁₋₆alkyleneNR¹⁰R¹¹, NR¹¹C₁₋₆alkyleneNR¹⁰R¹¹, NR¹¹C₁₋₆alkyleneOR¹⁰, NR^{10a}R^{11a}, NR¹¹COR¹⁰, NR¹¹CO₂R¹⁰, NR¹¹SO₂R¹⁰, C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to three of R¹³

In an embodiment, each R⁹ is independently selected from F, Cl, CN, NO₂, C₁₋₄alkyl, OR¹⁰, C(O)R¹⁰, CO₂R¹⁰, P(O)R¹⁰R¹¹, P(O)(OR¹⁰)(OR¹¹), SR¹⁰, S(O)R¹⁰, SO₂R¹⁰, S(O)(═NR¹¹)R¹⁰, SO₂NR¹⁰R¹¹, SiR¹²R^{12a}R^{12b}, C₁₋₄alkyleneOR¹⁰, OC₁₋₄alkyleneOR¹⁰, C₁₋₄alkyleneNR^{10a}R^{11a}, OC₁₋₄alkyleneNR¹⁰R¹¹, NR—C₁₋₄alkyleneNR¹⁰R¹¹, NR—C₁₋₄alkyleneOR¹⁰, NR^{10a}R^{11a} NR¹¹COR¹⁰, NR¹¹CO₂R¹⁰, NR¹¹SO₂R¹⁰, C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to three of R¹³.

In an embodiment, each R⁹ is independently selected from F, Cl, CN, NO₂, CH₃, CH₂CH₃, CH(CH₃)₂, OR¹⁰, C(O)R¹⁰, Co₂R¹⁰, P(O)R¹⁰R¹¹, P(O)(OR¹⁰)(OR¹¹), SR¹⁰, S(O)R¹⁰, SO₂R¹⁰, S(O)(═NR¹¹)R¹⁰, SO₂NR¹⁰R¹¹, SiR¹²R^{12a}R^{12b}, C₁₄alkyleneOR¹⁰, OC₁₋₄alkyleneOR¹⁰, C₁₋₄alkyleneNR^{10a}R^{11a} OC₁₋₄alkyleneNR¹⁰R¹¹, NR¹¹C₁₋₄alkyleneNR¹⁰R¹¹, NR¹¹C₁₋₄alkyleneOR¹⁰, NR^{10a}R^{11a}, NR¹¹COR¹⁰, NR¹¹CO₂R¹⁰, NR¹¹SO₂R¹⁰, C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to four of R¹³.

In an embodiment, each R⁹ is independently selected from F, CH₃, CH₂CH₃, CH(CH₃)₂, P(O)R¹⁰R¹¹, SO₂NR¹⁰R¹¹, SiR¹²R^{12a}R^{12b}, OC₁₋₄alkyleneOR¹⁰, C₁₋₄alkyleneNR^{10a}R^{11a} NR^{10a}R^{11a} NR¹¹COR¹⁰, NR¹¹SO₂R¹⁰, C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to three R¹³. In an embodiment, each R⁹ is independently selected from F, CH₃, CH₂CH₃, CH(CH₃)₂, SO₂NR¹⁰R¹¹, OC₁₋₄alkyleneOR¹⁰, NR¹⁻⁴alkyleneNR¹⁰R¹¹, C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to three of R¹³

In an embodiment, each R⁹ is independently selected from C₁₋₄alkyleneNR^{10a}R^{11a}, NR^{10a}R^{11a}, NR¹¹COR¹⁰, SO₂NR¹⁰R¹¹, NR¹¹C₁₋₄alkyleneNR¹⁰R¹¹, C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to four of R¹³ In an embodiment, one or two R⁹ are selected from C₁₋₄alkyleneNR^{10a}R^{11a}, NR^{10a}R^{11a}, NR¹¹COR¹⁰, SO₂NR¹⁰R¹¹, NR¹¹C₁₋₄alkyleneNR¹⁰R¹¹, C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to four of R¹³. In an embodiment, one or two R⁹ are selected from C₁₋₄alkyleneNR^{10a}R^{11a}, NR^{10a}R^{11a}, NR¹¹COR¹⁰, SO₂NR¹⁰R¹¹, NR¹¹C₁₋₄alkyleneNR¹⁰R¹¹, C₃₋₇heterocycloalkyl, and C₁₋₄alkyleneC₃₋₇heterocycloalkyl, the latter four groups being optionally substituted with one to four of R¹³. In an embodiment, one R¹¹ is SO₂NR¹⁰R¹¹. In an embodiment, one or two R⁹ are selected from C₁₋₄alkyleneNR^{10a}R^{11a}, NR^{10a}R^{11a}, NR¹¹COR¹⁰, NR¹¹C₁₋₄alkyleneNR¹⁰R¹¹, C₃₋₇heterocycloalkyl, and C₁₋₄alkyleneC₃₋₇heterocycloalkyl, the latter two groups being optionally substituted with one to four of R¹³. In an embodiment, one or two R⁹ are selected from C₁₋₄alkyleneNR^{10a}R^{11a}, NR^{10a}R^{11a}, C₃₋₇heterocycloalkyl, and C₁₋₄alkyleneC₃₋₇heterocycloalkyl, the latter two groups being optionally substituted with one to four of R¹³.

In an embodiment, each R⁹ is independently selected from C₁₋₄alkyleneNR^{10a}R^{11a}, NR^{10a}R^{11a}, NR¹¹COR¹⁰, SO₂NR¹⁰R¹¹, NR¹¹C₁₋₄alkyleneNR¹⁰R¹¹. In an embodiment, one of R⁹ is SO₂NR¹⁰R¹¹. In an embodiment, one of R⁹ is selected from C₁₋₄alkyleneNR^{10a}R^{11a}, NR^{10a}R^{11a}, and NR¹¹C₁₋₄alkyleneNR¹⁰R¹¹. In an embodiment, one or two R⁹ are selected from C₁₋₄alkyleneNR^{10a}R^{11a}, NR^{10a}R^{11a}, NR¹¹COR¹⁰, SO₂NR¹⁰R¹¹, NR¹¹C₁₋₄alkyleneNR¹⁰R¹¹. In an embodiment, one of R⁹ is SO₂NR¹⁰R¹¹. In an embodiment, one or two of R⁹ is selected from C₁₋₄alkyleneNR^{10a}R^{11a}, NR^{10a}R^{11a}, and NR¹¹C₁₋₄alkyleneNR¹⁰R¹¹. In an embodiment, one of R⁹ is NR¹¹C₁₋₄alkyleneNR¹⁰R¹¹. In an embodiment, one of R⁹ is selected from C₁₋₄alkyleneNR^{10a}R^{11a} and NR^{10a}R^{11a}. In an embodiment, one of R⁹ is C₁₋₄alkyleneNR^{10a}R^{11a}.

In an embodiment, one of R⁹ is NR^{10a}R^{11a} R^{10a} is selected from H and C₁₋₄alkyl wherein C₁₋₄alkyl is optionally substituted with one to three of R¹⁵ and R^{11a} is selected from H and C₁₋₄alkyl. In an embodiment, one of R⁹ is NR^{10a}R^{11a}, R^{10a} is selected from H, CH₃, CH₂CH₃, CF₂H, CF₃, CFH₂, CH₂CF₂H, CH₂CF₃ wherein CH₃, CH₂CH₃, CF₂H, CF₃, CFH₂, CH₂CF₂H, CH₂CF₃ are optionally substituted with one to three of R¹⁵ and R^{11a} is selected from H, CH₃, CH₂CH₃, CF₂H, CF₃, CFH₂, CH₂CF₂H and CH₂CF. In an embodiment, one of R⁹ is NR^{10a}R^{11a} and R¹⁰ and R^{11a} are independently selected from H, CH₃, CH₂CH₃, CF₂H, CF₃, CFH₂, CH₂CF₂H and CH₂CF₃. In an embodiment, one of R⁹ is NR^{10a}R^{11a} and R¹⁰ and R¹a are independently selected from H, CF₃, CH₃ and CH₂CH₃. Accordingly, in an embodiment, R⁹ is selected from NH₂, N(CH₃)₂, NH(CH₃), N(CH₃)(CH₂CH₂), NH(CH₂CH₂) and N(CH₂CH₂). In an embodiment, one of R⁹ is selected from NH₂, N(CH₃)₂ and NH(CH₃).

In an embodiment, one of R⁹ is C₁₋₄alkyleneNR^{10a}R^{11a} and R¹⁰ is selected from H, C₁₋₄alkyl, C₂₋₄alkenyl, C₂₋₄alkynyl, C₁₋₄alkyleneC₃₋₁₀cycloalkyl, C₁₋₆alkyleneC₃₋₁₀heterocycloalkyl, C₁₋₆alkyleneOR¹⁴, and C₁₋₆alkyleneNR^{14a}R^{14b}, and all alkyl, alkenyl, alkynyl, alkylene, heterocycloalkyl and cycloalkyl groups in R^{10a} are optionally substituted with one to three of R¹⁵ and R¹⁰ is selected from H and C₁₋₄alkyl. In an embodiment, one of R⁹ is C₁₋₄alkyleneNR^{10a}R^{11a} and R¹⁰ is selected from H, C₁₋₄alkyl, C₁₋₄alkyleneC₃₋₁₀cycloalkyl, C₁₋₄alkyleneC₃₋₁₀heterocycloalkyl, C₁₋₄alkyleneOR¹⁴, and C₁₋₄alkyleneNR^{14a}R^{14b} and each alkyl, alkylene, heterocycloalkyl and cycloalkyl groups of R¹⁰ are optionally substituted with one to three of R¹⁵ and R¹⁰ is selected from H and C₁₋₄alkyl.

In an embodiment, one of R⁹ is C₁₋₄alkyleneNR^{10a}R^{11a} and R¹⁰ is selected from H and C₁₋₄alkyl wherein C₁₋₄alkyl is optionally substituted with one to three of R¹⁵ and R^{11a} is selected from H and C₁₋₄alkyl. In an embodiment, one of R⁹ is C₁₋₄alkyleneNR^{10a}R^{11a}, R^{10a} is selected from H, CH₃, CH₂CH₃, CF₂H, CF₃, CFH₂, CH₂CF₂H, CH₂CF₃wherein CH₃, CH₂CH₃, CF₂H, CF₃, CFH₂, CH₂CF₂H, CH₂CF₃ are optionally substituted with one to three of R¹⁵ and R^{11a} is selected from H, CH₃, CH₂CH₃, CF₂H, CF₃, CFH₂, CH₂CF₂H and CH₂CF. In an embodiment, one of R⁹ is C₁₋₄alkyleneNR^{10a}R^{11a} and R^{10a} and R^{11a} are independently selected from H, CH₃, CH₂CH₃, CF₂H, CF₃, CFH₂, CH₂CF₂H and CH₂CF₃. In an embodiment, one of R⁹ is C₁₋₃alkyleneNR^{10a}R^{11a} and R¹⁰ and R^{11a} are independently selected from H, CF₃, CH₃ and CH₂CH₃. Accordingly, in an embodiment, R⁹ is CH₂N(CH₂CH₃)₂, C(CH₃)₂NH₂, CH₂N(CH₃)₂, CH₂CH₂N(CH₃)₂ and CH₂N(CH₃)₂ and CH₂N(CH₃)₂. In an embodiment, R⁹ is CH₂N(CH₃)₂. In an embodiment, one of R⁹ is C₁₋₃alkyleneNR^{10a}R^{11a} and R^{10a} and R^{11a} are independently selected from H and CH₃. In an embodiment, one of R⁹ is C₁₋₃alkyleneNR^{10a}R^{11a} and R¹⁰ and R^{11a} are both H or are both CH₃.

In an embodiment, one of R⁹ is C₁₋₄alkyleneNR^{10a}R^{11a} and R¹⁰ is selected from C₁₋₆alkyleneOR¹⁴ and C₁₋₆alkyleneNR^{14a}R^{14b} and all alkylene groups in R^{10a} are optionally substituted with one to three of R¹⁵. In an embodiment, one of R⁹ is C₁₋₄alkyleneNR^{10a}R^{11a} and R¹⁰ is selected from C₁₋₄alkyleneOR¹⁴ and C₁₋₄alkyleneNR^{14a}R^{14b}and all alkylene groups in R^{10a} are optionally substituted with one to three of R¹⁵. In an embodiment, one of R⁹ is C₁₋₄alkyleneNR^{10a}R^{11a} R^{10a} is selected from C₁₋₄alkyleneOR¹⁴ and C₁₋₄alkyleneNR^{14a}R^{14b} and R^{11a} is selected from H and C₁₋₄alkyl and all alkylene groups in R^{10a} are optionally substituted with one to three of R¹⁵. Accordingly, in an embodiment, one of R⁹ is C₁₋₄alkyleneN(R^{11a})(C₁₋₆alkyleneOR¹⁴) and R^{11a} is selected from H and C₁₋₄alkyl and all alkylene groups in R^{10a} are optionally substituted with one to three of R¹⁵.

In an embodiment, one of R⁹ is C₁₋₄alkyleneNR^{10a}R^{11a} R^{10a} is selected from C₁₋₄alkyleneOR¹⁴ and C₁₋₄alkyleneNR^{14a}R^{14b} and R^{11a} is selected from H and C₁₋₄alkyl. In an embodiment, one of R⁹ is selected from C₁₋₄alkyleneNR^{10a}R^{11a}, R^{10a} is C₁₋₄alkyleneOR¹⁴ and C₁₋₄alkyleneNR^{14a}R^{14b} and R^{11a} is selected from H, CH₃, CH₂CH₃, CF₂H, CF₃, CFH₂, CH₂CF₂H and CH₂CF₃. In an embodiment, one of R⁹ is C₁₋₄alkyleneNR^{10a}R^{11a}, R^{10a} is selected from C₁₋₄alkyleneOR¹⁴ and C₁₋₄alkyleneNR^{14a}R^{14b} and R^{11a} is selected from H, CH₃ and CH₂CH₃. In an embodiment, one of R⁹ is C₁₋₄alkyleneNR^{10a}R^{11a} R^{10a} is selected from C₁₋₄alkyleneOR¹⁴ and R^{11a} is selected from H and CH₃.

In an embodiment, one of R⁹ is C₁₋₄alkyleneNR^{10a}R^{11a} and R¹⁰ is selected from C₁₋₆alkyleneOR¹⁴ and R¹⁴ in R^{10a} is selected from H and C₁₋₄alkyl. In an embodiment, one of R⁹ is C₁₋₄alkyleneNR^{10a}R^{11a} and R^{10a} is selected from C₁₋₆alkyleneOR¹⁴ and R¹⁴ is selected from H, CH₃, CH₂CH₃, CF₂H, CF₃, CFH₂, CH₂CF₂H and CH₂CF₃. In an embodiment, one of R⁹ is C₁₋₄alkyleneNR^{10a}R^{11a} and R^{10a} is selected from C₁₋₆alkyleneOR¹⁴ and R¹⁴ is selected from H, CF₃ and CH₃. In an embodiment, one of R⁹ is C₁₋₄alkyleneNR^{10a}R^{11a} and R^{10a} is selected from C₁₋₆alkyleneOR¹⁴ and each R¹⁴ is selected from H and CH₃.

Accordingly, in an embodiment, one of R⁹ is selected from C₁₋₄alkyleneNH(C₁₋₆ alkyleneOH), C₁₋₄alkyleneNCH₃(C₁₋₆alkyleneOH), C₁₋₄alkyleneNH(C₁₋₆alkyleneOCH₃), C₁₋₄alkyleneNCH₃(C₁₋₆alkyleneOCH₃). In an embodiment, one of R⁹ is selected from C₁₋₄alkyleneNH(C₁₋₄alkyleneOH), C₁₋₄alkyleneNCH₃(C₁₋₆alkyleneOH), C₁₋₄alkyleneNH(C₁₋₄ alkyleneO CH₃) and C₁₋₄alkyleneNCH₃(C₁₋₆alkyleneOCH₃). In an embodiment, one of R⁹ is C₁₋₄alkyleneNR^{10a}R^{11a} and R^{10a} is C₁₋₆alkyleneC₃₋₇heterocycloalkyl and R^{11a} is selected from H and C₁₋₄alkyl. In an embodiment, one of R⁹ is selected from C₁₋₄alkyleneC₃₋₇heterocycloalkyl and R^{10a} is C₁₋₄alkyleneC₃₋₇heterocycloalkyl and R^{11a} is selected from H and C₁₋₄alkyl. In an embodiment, one of R⁹ is C₁₋₄alkyleneC₃₋₇heterocycloalkyl and R¹⁰ is C₁₋₂alkyleneC₃₋₇heterocycloalkyl and R^{11a} is selected from H and C₁₋₄alkyl. In an embodiment, the C₃₋₇heterocycloalkyl in the C₁₋₄alkyleneC₃₋₇heterocycloalkyl of R¹⁰ is selected from azetidinyl, oxetanyl, tetrahydrofuranyl, tetrahydrothiophenyl, pyrrolidinyl, pyrrolidin-2-onyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, tetrahydropyranyl, thiomorpholinyl, morpholinyl, dioxanyl, azepanyl, diazepanyl, oxepanyl, thiepanyl, azabicyclohexanyl, azabicycloheptanyl, oxabicyclohexanyl, and oxabicycloheptanyl. In an embodiment, the C₃₋₇heterocycloalkyl in the C₁₋₄alkyleneC₃₋₇heterocycloalkyl of R¹⁰ is selected from selected from azetidinyl, tetrahydrofuranyl, pyrrolidinyl, pyrrolidin-2-onyl, piperidinyl, piperazinyl and morpholinyl.

In an embodiment, each R⁹ is independently selected from C₃₋₇cycloalkyl and C₃₋₇heterocycloalkyl, C₁₋₄alkyleneC₃₋₇cycloalkyl and C₁₋₄alkyleneC₃₋₇heterocycloalkyl, optionally substituted with one to four of R¹³. In an embodiment, one or two R⁹ are selected from C₃₋₇cycloalkyl, C₃₋₇heterocycloalkyl, C₁₋₄alkyleneC₃₋₇cycloalkyl and C₁₋₄alkyleneC₃₋₇heterocycloalkyl, each of which is optionally substituted with one to four of R¹³. In an embodiment, one R⁹ is selected from C₁₋₃alkyleneC₃₋₇cycloalkyl and C₃₋₇cycloalkyl, optionally substituted with one or two of R¹³. In an embodiment, the C₃₋₇cycloalkyl in the C₁₋₃alkyleneC₃₋₇cycloalkyl and C₃₋₇cycloalkyl of R⁹ is selected from C₃₋₇cycloalkyl selected from cyclopropyl, cyclobutyl, cyclopentyl, cyclohexyl, cycloheptyl, bicyclo[2.2.1]hexanyl and bicyclo[2.2.1]heptanyl, optionally substituted with one or two of R¹³

In an embodiment, one of R⁹ is selected from C₁₋₃alkyleneC₃₋₇cycloalkyl and C₃₋₇cycloalkyl, optionally substituted with one to three of R¹³. In an embodiment, the C₃₋₇cycloalkyl in the C₁₋₃alkyleneC₃₋₇cycloalkyl and C₃₋₇cycloalkyl of R⁹ is selected from cyclopropyl, cyclobutyl, cyclopentyl, cyclohexyl, cycloheptyl, bicyclo[2.2.1]hexanyl and bicyclo[2.2.1]heptanyl, optionally substituted with one or two of R¹³.

In an embodiment, one of R⁹ is selected from C₃₋₇heterocycloalkyl and C₁₋₄alkyleneC₃₋₇heterocycloalkyl, each of which is optionally substituted with one to four of R¹³ In an embodiment, the C₃₋₇heterocycloalkyl in the C₃₋₇heterocycloalkyl and C₁₋₄alkyleneC₃₋₇heterocycloalkyl of R⁹ is selected from azetidinyl, oxetanyl, tetrahydrofuranyl, tetrahydrothiophenyl, pyrrolidinyl, pyrrolidin-2-onyl, azabicyclohexanyl, azabicycloheptanyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, tetrahydropyranyl, thiomorpholinyl, morpholinyl, dioxanyl, azepanyl, diazepanyl, oxepanyl, thiepanyl, azabicyclohexanyl, azabicycloheptanyl, oxabicyclohexanyl, and oxabicycloheptanyl optionally substituted with one to three of R¹³. In an embodiment, one of R⁹ is selected from C₄₋₆heterocycloalkyl and C₁₋₄alkyleneC₄₋₆heterocycloalkyl, and the C₄₋₆heterocycloalkyl in the C₄₋₆heterocycloalkyl and C₁₋₄alkyleneC₄₋₆heterocycloalkyl of R⁹ is selected from azetidinyl, oxetanyl, tetrahydrofuranyl, tetrahydrothiophenyl, pyrrolidinyl, pyrrolidin-2-onyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, tetrahydropyranyl, thiomorpholinyl, morpholinyl, and dioxanyl, optionally substituted with one to three of R¹³. In an embodiment, the C₄₋₆heterocycloalkyl in the C₄₋₆heterocycloalkyl and C₁₋₄alkyleneC₄₋₆heterocycloalkyl of R⁹ is selected from tetrahydrofuranyl, tetrahydrothiophenyl, pyrrolidinyl, pyrrolidin-2-onyl, imidazolidinyl, pyrazolidinyl, thiazolidinyl and isothiazolidinyl, optionally substituted with one to three of R¹³ In an embodiment, the C₄₋₆heterocycloalkyl in the C₄₋₆heterocycloalkyl and C₁₋₄alkyleneC₄₋₆ heterocycloalkyl of R⁹ is selected from pyrrolidinyl, pyrrolidin-2-onyl, imidazolidinyl, pyrazolidinyl, thiazolidinyl, and isothiazolidinyl, optionally substituted with one to three of R¹³

In an embodiment, each R⁹ is independently selected from C₃₋₇heterocycloalkyl and C₁₋₄alkyleneC₃₋₇heterocycloalkyl, optionally substituted with one to four of R¹³. In an embodiment, the C₃₋₇heterocycloalkyl in the C₃₋₇heterocycloalkyl and C₁₋₄alkyleneC₃₋₇heterocycloalkyl of R⁹ is selected from azetidinyl, oxetanyl, tetrahydrofuranyl, tetrahydrothiophenyl, pyrrolidinyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, tetrahydropyranyl, thiomorpholinyl, morpholinyl, dioxanyl, azepanyl, diazepanyl, oxepanyl, thiepanyl, azabicyclohexanyl, azabicycloheptanyl, oxabicyclohexanyl, and oxabicycloheptanyl optionally substituted with one to three of R¹³. In an embodiment, one of R⁹ is selected from C₄₋₆heterocycloalkyl and C₁₋₄alkyleneC₄₋₆heterocycloalkyl, and the C₄₋₆heterocycloalkyl in the C₄₋₆heterocycloalkyl and C₁₋₄alkyleneC₄₋₆heterocycloalkyl of R⁹ is selected from azetidinyl, oxetanyl, tetrahydrofuranyl, tetrahydrothiophenyl, pyrrolidinyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, tetrahydropyranyl, thiomorpholinyl, morpholinyl, and dioxanyl, optionally substituted with one to three of R¹³. In an embodiment, the C₄₋₆heterocycloalkyl in the C₄₋₆heterocycloalkyl and C₁₋₄alkyleneC₄₋₆heterocycloalkyl of R⁹ is selected from tetrahydrofuranyl, tetrahydrothiophenyl, pyrrolidinyl, imidazolidinyl, pyrazolidinyl, thiazolidinyl and isothiazolidinyl, optionally substituted with one to three of R¹³. In an embodiment, the C₄₋₆heterocycloalkyl in the C₄₋₆heterocycloalkyl and C₁₋₄alkyleneC₄₋₆heterocycloalkyl of R⁹ is selected from pyrrolidinyl, imidazolidinyl, pyrazolidinyl, thiazolidinyl, and isothiazolidinyl, optionally substituted with one to three of R¹³. In some embodiments, the C₄₋₆heterocycloalkyl in the C₄₋₆heterocycloalkyl and C₁₋₄alkyleneC₄₋₆heterocycloalkyl of R¹³ is selected from pyrrolidin-2-onyl, azabicyclohexanyl and azabicycloheptanyl. In some embodiments, the C₄₋₆heterocycloalkyl in the C₄₋₆heterocycloalkyl and C₁₋₄alkyleneC₄₋₆heterocycloalkyl of R¹³ is pyrrolidin-2-onyl.

In an embodiment, the C₃₋₇heterocycloalkyl in the C₃₋₇heterocycloalkyl and C₁₋₄alkyleneC₃₋₇heterocycloalkyl of R⁹ comprises at least one N atom. In an embodiment, the C₃₋₇heterocycloalkyl in the C₃₋₇heterocycloalkyl and C₁₋₄alkyleneC₃₋₇heterocycloalkyl of R⁹ is selected from azetidinyl, pyrrolidinyl, pyrrolidin-2-onyl, azabicyclohexanyl, azabicycloheptanyl, piperidinyl, piperazinyl and morpholinyl each of which optionally substituted with one or two of R¹³. In an embodiment, the C₄₋₆heterocycloalkyl in the C₄₋₆heterocycloalkyl and C₁₋₄alkyleneC₄₋₆heterocycloalkyl of R⁹ is pyrrolidinyl, optionally substituted with one or two of R¹³.

In an embodiment, one of R⁹ is C₃₋₇heterocycloalkyl, optionally substituted with one to four of R¹³ and the C₃₋₇heterocycloalkyl is selected from azabicyclohexanyl, azabicycloheptanyl, pyrrolidinyl and pyrrolidin-2-onyl optionally substituted with one to four of R¹³. In an embodiment, one of R⁹ is C₄₋₆heterocycloalkyl, optionally substituted with one to four of R¹³ and the C₄₋₆heterocycloalkyl is pyrrolidin-2-onyl. In an embodiment, one of R⁹ is pyrrolidin-2-onyl selected from

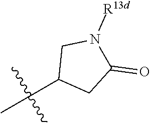

optionally substituted with one to three of R¹³, wherein R^{13d} is selected from H and R¹³, and [symbol] indicates a point of covalent attachment to Cy¹. In an embodiment, one of R⁹ is C₄₋₆heterocycloalkyl, optionally substituted with one to four of R¹³ and the C₄₋₆heterocycloalkyl is pyrrolidinyl. In an embodiment, one of R⁹ is pyrrolidine selected from

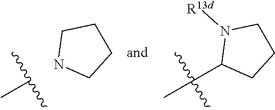

and optionally substituted with one to three of R¹³, wherein R^{13d} is selected from H and R¹³, and [symbol] indicates a point of covalent attachment to Cy¹. In an embodiment, one of R⁹ is C₄₋₆heterocycloalkyl, and the C₄₋₆heterocycloalkyl is pyrrolidinyl selected from

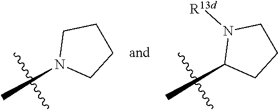

optionally substituted with one or two of R¹³, wherein R^{13d} is selected from H and R¹³, and [symbol] indicates a point of covalent attachment to Cy¹. In an embodiment, R^{13d} is H. In an embodiment, R^{13d} is R¹³.

In an embodiment, one of R⁹ is C₁₋₆alkyleneC₃₋₇heterocycloalkyl, optionally substituted with one to four of R¹³, and the C₃₋₇heterocycloalkyl in the C₁₋₆alkyleneC₃₋₇heterocycloalkyl is selected from azetidinyl, pyrrolidinyl, pyrrolidin-2-onyl, piperidinyl, piperazinyl and morpholinyl. In an embodiment, one of R⁹ is C₁₋₃alkyleneC₄₋₇heterocycloalkyl, optionally substituted with one to four of R¹³, and the C₄₋₇heterocycloalkyl in the C₁₋₆alkyleneC₃₋₇heterocycloalkyl is selected from azetidinyl, pyrrolidinyl, piperidinyl, piperazinyl and morpholinyl. Accordingly, in an embodiment, one of R⁹ is selected from C₁₋₃alkyleneazetidinyl, C₁₋₃alkylenepyrrolidinyl, C₁₋₃alkylenepiperidinyl, C₁₋₃alkylenepiperazinyl and C₁₋₃alkylenemorpholinyl optionally substituted with one to four of R¹³. In an embodiment, one of R⁹ is selected from CH₂azetidinyl, CH₂pyrrolidinyl, CH₂piperidinyl, CH₂piperazinyl and CH₂morpholinyl optionally substituted with one to four of R¹³. In an embodiment, one of R⁹ is selected from

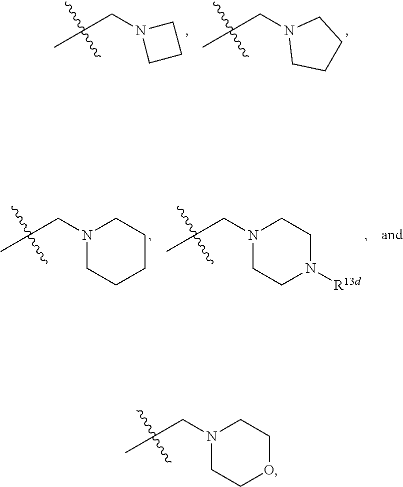

optionally substituted with one or two of R¹³, wherein R^{13d} is selected from H and R¹³, and [symbol] indicates a point of covalent attachment to Cy¹.

In an embodiment, one of R⁹ is C₁₋₆alkyleneC₃₋₇heterocycloalkyl, optionally substituted with one to four of R¹³, and the C₃₋₇heterocycloalkyl in the C₁₋₆alkyleneC₃₋₇heterocycloalkyl is pyrrolidinyl. In an embodiment, one of R⁹ is C₁₋₄alkylenepyrrolidinyl optionally substituted with one to three of R¹³. In an embodiment, one of R⁹ is C₁₋₄alkylenepyrrolidinyl optionally substituted with one or two of R¹³, wherein a R¹³ is F. In an embodiment, one of R⁹ is selected from CH₂pyrrolidinyl optionally substituted with one to four of R¹³. In an embodiment, the one of R⁹ is

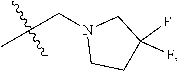 [SMILES: *CN1CCC(F)(F)C1]

wherein [symbol] indicates a point of covalent attachment to Cy¹.

In an embodiment, each R¹³ is independently selected from F, Cl, CN, OH, C₁₋₄alkyl, OC₁₋₄alkyl and NR^{13a}R^{13b}. In an embodiment, each R¹³ is independently selected from F, Cl, OC₁₋₄alkyl, C₁₋₄alkyl and NR^{13a}R^{13b}.

In an embodiment, one R¹³ is selected from OH, OC₁₋₄alkyl and NR^{13a}R^{13b}In an embodiment, one of R¹³ is selected from OH and OC₁₋₄alkyl. In an embodiment, one R¹³ is OC₁₋₄alkyl. In an embodiment, one of R¹³ is OCH₃.

In an embodiment, one of R⁹ is C₁₋₆alkyleneC₃₋₇heterocycloalkyl as described above, substituted with one or two R¹³ and one R¹³ is selected from OH and OC₁₋₄alkyl. In an embodiment, one of R⁹ is C₁₋₆alkyleneC₃₋₇heterocycloalkyl as described above, substituted with one R¹³ and R¹³ is OCH₃.

In an embodiment, each R¹³ is independently selected from F, Cl, CN, C₁₋₄alkyl and NR^{13a}R^{13b}. In an embodiment, each R¹³ is independently selected from F, C₁₋₄alkyl and NR^{13a}R^{13b}. In an embodiment, one or two R¹³ is independently selected from F, Cl, CN, C₁₋₄alkyl and NR^{13a}R^{13b}. In an embodiment, one or two R¹³ is selected from F, C₁₋₄alkyl and NR^{13a}R^{13b}. In an embodiment, one of R¹³ is F. In an embodiment, one of R¹³ is NR^{13a}R^{13b}

In an embodiment, R^{13a} and R^{13b} are independently selected from H and C₁₋₄alkyl. In an embodiment, R^{13a} and R^{13b} are independently selected from H, CH₃, CH₂CH₃, CF₂H, CF₃, CFH₂, CH₂CF₂H and CH₂CF₃. In an embodiment, R^{13a}and R^{13b} are independently selected from H, CF₃ and CH₃. In an embodiment, R^{13a} and R^{13b} are independently selected from H and CH₃.

In an embodiment, one of R⁹ is selected from C₃₋₇cycloalkyl, C₃₋₇heterocycloalkyl, C₁₋₄alkyleneC₃₋₇cycloalkyl and C₁₋₄alkyleneC₃₋₇heterocycloalkyl which are unsubstituted. In an embodiment, one of R⁹ is C₃₋₇heterocycloalkyl as defined above which is unsubstituted. In an embodiment, one of R⁹ is selected from pyrrolidinyl, pyrrolidin-2-onyl, imidazolidinyl, pyrazolidinyl, thiazolidinyl, and isothiazolidinyl, which is unsubstituted. In an embodiment, one of R⁹is pyrrolidinyl which is unsubstituted. In an embodiment, one of R⁹is selected from pyrrolidinyl, imidazolidinyl, pyrazolidinyl, thiazolidinyl, and isothiazolidinyl, which is unsubstituted. In an embodiment, one of R⁹is pyrrolidinyl which is unsubstituted. In an embodiment, Cy¹ is substituted with one R⁹ and R⁹ is pyrrolidin-2-only which is unsubstituted.

In an embodiment, one of R⁹ is selected from

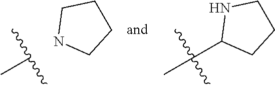

wherein [symbol] indicates a point of covalent attachment to Cy¹. In an embodiment, one of R⁹ is selected from

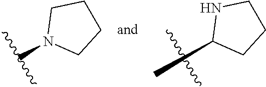

wherein [symbol] indicates a point of covalent attachment to Cy¹.

In an embodiment, one to three R⁹ are independently selected from F, Cl, CN, NO₂ and C₁₋₄alkyl. In an embodiment, each R⁹ is independently selected from F, Cl, CN, NO₂, CH₃, CH₂CH₃, and CH(CH₃)₂. In an embodiment, one to three R⁹are independently selected from F, Cl, CN, NO₂, CH₃, CH₂CH₃, CH₂CH₂CH₃, CH₂CH₂CH₂CH₃, CH(CH₃)₂, CH₂CH(CH₃)₂, C(CH₃) and CH(CH₃)₂. In an embodiment, each R⁹ is independently selected from F, Cl, CN, CH₃, CH₂CH₃, CF₂H, CF₃, CFH₂, CH₂CH₂F, CH₂CF₂H, and CH(CH₃)₂. In an embodiment, one to three R⁹ are independently selected from F, Cl, CN, CH₃, CH₂CH₃, CH₂CH₂CH₃, CH(CH₃)₂, CH₂CH₂CH₂CH₃, CF₂H, CF₃, CFH₂, CH₂CH₂F, CH₂CF₂H, CH₂CH₂F₂H, CH₂CH₂CH₂F₂H and CH(CH₃)₂. In an embodiment, each R⁹ is independently selected from F, C, CN, CH₃, CH₂CH₃, CF₃, CH₂CF₂H, CH₂CF₃ and CH(CH₃)₂ In an embodiment, one to three R⁹ are independently selected from F, C, CN, CH₃, CH₂CH₃, CF₃, CF₂H, CH₂CF₂H, CH₂CF₃, CH₂CH₂F₂H, CH₂CH₂CH₂F₂H and CH(CH₃)₂ In an embodiment, one to three R⁹ are independently selected from CH₃, CH₂CH₃, CF₃, CF₂H, CH₂CF₂H, CH₂CF₃, CH₂CH₂F₂H, CH₂CH₂CH₂F₂H and CH(CH₃)₂. In an embodiment, one or two R⁹ are selected from CF₂H, CH₂CF₂H, CH₂CH₂F₂H, and CH₂CH₂CH₂F₂H. In an embodiment, one or two R⁹ are selected from CH₂CH₂F₂H and CH₂CH₂CH₂F₂H. In an embodiment, each R⁹ is independently selected from F, C, CN, CH₃, CH₂CH₃, CF₃, CH₂CF₂H, CH₂CF₃, and CH(CH₃)₂. In an embodiment, each R⁹ is independently selected from F, CN, CH₃, CH₂CH₃, CF₃, CH₂CF₂H, CH₂CF₃, and CH(CH₃)₂. In an embodiment each R⁹is independently selected from CF₃, CH₂CF₃, and F. In an embodiment, one to three R⁹ are independently selected from F, C, CN, CH₃, CH₂CH₃, CF₃, CH₂CF₂H, CH₂CF₃, and CH(CH₃)₂. In an embodiment, one to three R⁹ are independently selected from F, CN, CH₃, CH₂CH₃, CF₃, CH₂CF₂H, CH₂CF₃, and CH(CH₃)₂. In an embodiment, one R⁹is CN. In an embodiment, one to three R⁹ are independently selected from CF₃, CH₂CF₃, and F. In an embodiment, one to three R⁹ are independently selected from CF₂H, CH₂CF₂H, CH₂CF₂H, CH₂CH₂F₂H and CH₂CH₂CH₂F₂H. In an embodiment, one to three of R⁹ is F. In an embodiment, one of R⁹ is F.

In an embodiment, each R⁹ is independently selected from F, CH₃, CH₂CH₃, CH(CH₃)₂, OR¹⁰, C(O)R¹⁰, CO₂R¹⁰, SR¹⁰, SO₂R¹⁰, S(O)(═NR¹¹)R¹⁰, C₁₋₄alkyleneOR¹⁰, OC₁₋₄alkyleneOR¹⁰, C₁₋₄alkyleneNR^{10a}R^{11a}, OC₁₋₄alkyleneOR¹⁰, NR¹¹C₁₋₄alkyleneOR¹⁰, NR¹¹COR¹⁰, and NR¹¹SO₂R¹⁰, and R¹⁰ is selected from H, CH₃, CH₂CH₃, CF₂H, CF₃, CFH₂, CH₂CF₂H, CH₂CF₃, C₁₋₂alkyleneC₃₋₁₀cycloalkyl, C₁₋₂alkyleneC₃₋₁₀heterocycloalkyl, C₁₋₂alkylalkyleneOR¹⁴, and C₁₋₂alkylalkyleneNR^{14a}R^{14b} and all alkyl, alkenyl, alkynyl, alkylene and cycloalkyl groups of R¹⁰ are optionally substituted with one to three of R¹⁵. In an embodiment, one to three R⁹ are independently selected from OR¹⁰, C(O)R¹⁰, CO₂R¹⁰, SR¹⁰, SO₂R¹⁰, S(O)(═NR¹¹)R¹⁰, C₁₋₄alkyleneOR¹⁰, OC₁₋₄alkyleneOR¹⁰, NR¹¹C₁₋₄alkyleneOR¹⁰, NR¹¹COR¹⁰, and NR¹¹SO₂R¹⁰

In an embodiment, R¹⁰ and R^{10a} are independently selected from H, C₁₋₄alkyl, C₂₋₄alkenyl, C₂₋₄alkynyl, C₁₋₄alkyleneC₃₋₁₀cycloalkyl, C₁₋₆alkyleneC₃₋₁₀heterocycloalkyl, C₁₋₆alkylalkyleneOR¹⁴, and C₁₋₆alkyleneNR^{14a}R^{14b}, and all alkyl, alkenyl, alkynyl, alkylene, ₁heterocycloalkyl and cycloalkyl groups of R¹⁰ or R^{10a} are optionally substituted with one to three of R¹⁵. In an embodiment, R¹⁰ and R^{10a} are independently selected from H, C₁₋₄alkyl, C₁₋₄alkyleneC₃₋₁₀cycloalkyl, C₁₋₄alkyleneC₃₋₁₀heterocycloalkyl, C₁₋₄alkyleneOR¹⁴, and C₁₋₄alkylalkyleneNR^{14a}R^{14b} and alkyl, alkenyl, alkynyl, alkylene, heterocycloalkyl and cycloalkyl groups of R¹⁰ or R^{10a} are optionally substituted with one to three of R¹⁵. In an embodiment, R¹⁰ and R^{10a} are independently selected from H, CH₃, CH₂CH₃, CF₂H, CF₃, CFH₂, CH₂CF₂H, CH₂CF₃, C₁₋₂alkyleneC₃₋₁₀cycloalkyl, C₁₋₂alkyleneC₃₋₁₀heterocycloalkyl, C₁₋₂alkyleneOR¹⁴, and C₁₋₂alkyleneNR^{14a}R^{14b} and all alkyl, alkenyl, alkynyl, alkylene heterocycloalkyl and cycloalkyl groups of R¹⁰ or R^{10a} are optionally substituted with one to three of R¹⁵. In an embodiment, each R¹⁰ is selected from H, CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃), C₁₋₂alkyleneC₃₋₁₀cycloalkyl, C₁₋₂alkyleneC₃₋₁₀heterocycloalkyl, C₁₋₂alkyleneOR¹⁴, and C₁₋₂alkyleneNR^{14a}R^{14b} and CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃), C₁₋₂alkyleneC₃₋₁₀cycloalkyl, C₁₋₂alkyleneC₃₋₁₀heterocycloalkyl, C₁₋₂alkyleneOR¹⁴, and C₁₋₂alkyleneNR^{14a}R^{14b}are optionally substituted with one to three of R¹⁵.

In an embodiment, R¹¹ and R^{11a} are independently selected from H and C₁₋₄alkyl. In an embodiment, R¹¹ and R^{11a} are independently selected from H, CH₃, CH₂CH₃, CF₂H, CF₃, CFH₂, CH₂CF₂H and CH₂CF₃. In an embodiment, R¹¹ and R^{11a} are independently selected from H, CF₃ and CH₃. In an embodiment, R¹¹ and R^{11a} are independently selected from H and CH₃. In an embodiment, each R¹¹ is independently selected from H and C₁₋₄alkyl. In an embodiment, each R¹¹ is independently selected from H, CH₃, CH₂CH₃, CH(CH₃)₂, CH₂CH(CH₃)₂, C(CH₃)₃, CF₂H, CF₃, CFH₂, CH₂CF₂H and CH₂CF₃. In an embodiment, each R¹¹ is independently selected from H, CF₃ and CH₃. In an embodiment, each R¹¹ is independently selected from H and CH₃.

In an embodiment, R¹⁰ and R¹¹ are joined to form, together with the atom(s) therebetween, a 4- to 6-membered saturated or unsaturated ring, optionally containing one additional heteromoiety selected from N, NR¹⁶, O, S, SO, and SO₂ and optionally substituted with one to three of R¹⁵. In an embodiment, R¹⁰ and R¹¹are joined to form, together with the atom(s) therebetween, to form a 4- to 6-membered heterocycloalkyl ring selected from diazetidinyl, pyrrolidinyl, imidazolidinyl, pyrazolidinyl, thiazolidinyl, isothiozolidinyl, piperidinyl, diazinanyl (e.g. piperazinyl), and morpholinyl, and optionally substituted with one to three of R¹⁵. In an embodiment, R¹⁰ and R¹¹ are joined to form, together with the atom(s) therebetween, to form a 5- to 6-membered heterocycloalkyl ring selected from pyrrolidinyl, imidazolidinyl, pyrazolidinyl, thiazolidinyl, isothiozolidinyl, piperidinyl, diazinanyl (e.g. piperazinyl) and morpholinyl, and optionally substituted with one to three of R¹⁵.

In an embodiment, one or two R⁹ are independently selected from OR¹⁰, C(O)R¹⁰, CO₂R¹⁰, SR¹⁰, SO₂R¹⁰, S(O)(═NR¹¹)R¹⁰, C₁₋₄alkyleneOR¹⁰, OC₁₋₄alkyleneOR¹⁰, NR¹C₁₋₄alkyleneOR¹⁰, NR¹¹COR¹⁰, and NR¹¹SO₂R¹⁰ and each R¹⁰ is independently selected from H, C₁₋₄alkyl, C₁₋₄alkyleneC₃₋₁₀cycloalkyl, C₁₋₄alkyleneC₃₋₁₀heterocycloalkyl, C₁₋₄alkyleneOR¹⁴, and C₁₋₄alkyleneNR^{14a}R^{14b} and alkylene, heterocycloalkyl and cycloalkyl groups of R¹⁰ are optionally substituted with one to three of R¹⁵ and R¹¹ is selected from H and C₁₋₄alkyl. In an embodiment, one or two R⁹ are independently selected from OR¹⁰, C(O)R¹⁰, CO₂R¹⁰, SR¹⁰, SO₂R¹⁰, S(O)(═NR¹¹)R¹⁰, C₁₋₄alkyleneOR¹⁰, OC₁₋₄alkyleneOR¹⁰, NR¹C₁₋₄alkyleneOR¹⁰, NR¹¹COR¹⁰, and NR¹¹SO₂R¹⁰ and R¹⁰ is selected from H, CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃)₃, C₁₋₂alkyleneC₃₋₁₀cycloalkyl, C₁₋₂alkyleneC₃₋₁₀heterocycloalkyl, C₁₋₂alkyleneOR¹⁴, and C₁₋₂alkyleneNR^{14a}R^{14b} and CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃)₃, C₁₋₂alkyleneC₃₋₁₀cycloalkyl, C₁₋₂alkyleneC₃₋₁₀heterocycloalkyl, C₁₋₂alkyleneOR¹⁴, and C₁₋₂alkyleneNR^{14a}R^{14b}groups of R¹⁰ are optionally substituted with one to three of R¹⁵ and R¹¹ is selected from H and C₁₋₄alkyl.

In an embodiment, one or two R⁹ are independently selected from SR¹⁰, SO₂R¹⁰, S(O)(═NR¹¹)R¹⁰, NR¹¹COR¹⁰, and NR¹¹SO₂R¹⁰ and R¹⁰ is selected from H, CH₃, CH₂CH₃, CF₂H, CF₃, CFH₂, CH₂CF₂H, CH₂CF₃, CH(CH₃)₂, CH₂CH(CH₃)₂, C(CH₃), C₁₋₂alkyleneC₃₋₁₀cycloalkyl, C₁₋₂alkyleneC₃₋₁₀heterocycloalkyl, C₁₋₂alkyleneOR¹⁴, and C₁₋₂alkyleneNR^{14a}R^{14b} and CH₃, CH₂CH₃, CF₂H, CF₃, CFH₂, CH₂CF₂H, CH₂CF₃, CH(CH₃)₂, CH₂CH(CH₃)₂, C(CH₃), C₁₋₂alkyleneC₃₋₁₀cycloalkyl, C₁₋₂alkyleneC₃₋₁₀heterocycloalkyl, C₁₋₂alkyleneOR¹⁴, and C₁₋₂alkyleneNR^{14a}R^{14b} groups of R¹⁰ are optionally substituted with one to three of R¹⁵ and R¹¹ is selected from H and C₁₋₄alkyl.

In an embodiment, one or two R⁹ are independently selected from SR¹⁰ and SO₂R¹⁰, R¹⁰ is selected from H, CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃), C₁₋₂alkyleneC₃₋₁₀cycloalkyl, C₁₋₂alkyleneC₃₋₁₀heterocycloalkyl, C₁₋₂alkyleneOR¹⁴, and C₁₋₂alkyleneNR^{14a}R^{14b} and all CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃)₃, C₁₋₂alkyleneC₃₋₁₀cycloalkyl, C₁₋₂alkyleneC₃₋₁₀heterocycloalkyl, C₁₋₂alkyleneOR¹⁴, and C₁₋₂alkyleneNR^{14a}R^{14b} groups of R¹⁰ are optionally substituted with one to three of R¹⁵. In an embodiment, one or two R⁹ are independently selected from SR¹⁰ and SO₂R¹⁰, R¹⁰ is selected from H, CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂ and C(CH₃)₃wherein CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃)₃ are optionally substituted with one to three of R¹⁵.

In an embodiment, one or two R⁹ are independently selected from OR¹⁰, C(O)R¹⁰, CO₂R¹⁰, C₁₋₄alkyleneOR¹⁰, OC₁₋₄alkyleneOR¹⁰, NR¹C₁₋₄alkyleneOR¹⁰ and R¹⁰ is selected from H, CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃)₃, C₁₋₂alkyleneC₃₋₁₀cycloalkyl, C₁₋₂alkyleneC₃₋₁₀heterocycloalkyl, C₁₋₂alkyleneOR¹⁴, and C₁₋₂alkyleneNR^{14a}R^{14b} and all CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃)₃, C₁₋₂alkyleneC₃₋₁₀cycloalkyl, C₁₋₂alkyleneC₃₋₁₀heterocycloalkyl, C₁₋₂alkyleneOR¹⁴, and C₁₋₂alkyleneNR^{14a}R^{14b} groups of R¹⁰ are optionally substituted with one to three of R¹⁵ and R¹¹ is selected from H and C₁₋₄alkyl.

In an embodiment, one or two R⁹ are independently selected from OR¹⁰, C(O)R¹⁰, CO₂R¹⁰, C₁₋₄alkyleneOR¹⁰, OC₁₋₄alkyleneOR¹⁰, OC₁₋₄alkyleneOR¹⁰, NR¹¹C₁₋₄alkyleneOR¹⁰ and R¹⁰ is selected from H, CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, and C(CH₃)₃ and wherein CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, and C(CH₃)₃ in R¹⁰ are optionally substituted with one to three of R¹⁵ and R¹¹ is selected from H and C₁₋₄alkyl. In an embodiment, one or two R⁹ are independently selected from OR¹⁰, C(O)R¹⁰, CO₂R¹⁰, C₁₋₄alkyleneOR¹⁰ and OC₁₋₄alkyleneOR¹⁰, and each R¹⁰ is selected from H, CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, and C(CH₃)₃ wherein the CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, and C(CH₃)₃ in R¹⁰ are optionally substituted with one to three of R¹⁵. In an embodiment, one R⁹ is selected from OR¹⁰, C₁₋₄alkyleneOR¹⁰, OC₁₋₄alkyleneOR¹⁰, and R¹⁰ is selected from H, CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, and C(CH₃)₃ wherein the CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, and C(CH₃)₃ in R¹⁰ are optionally substituted with one to three of R¹⁵. In an embodiment, one R⁹ is selected from OH, OCH₃, OCFH₂, OCH₂CF₂H, OCH₂CH₂F₂H, OCH₂CH₂CH₂F₂H, C₁₋₄alkyleneOH, C₁₋₄alkyleneOCH₃, OC₁₋₄alkyleneOH and OC₁₋₄alkyleneOCH₃. In an embodiment, one R⁹ is selected from OH, OCH₃, OCFH₂, OCH₂CF₂H, OCH₂CH₂F₂H, OCH₂CH₂CH₂F₂H, CH₂OH, CH₂CH₂OH, CH₂CH₂OCH₃, CH₂OCH₃, OCH₂CH₂OH and OCH₂CH₂OCH₃.

In an embodiment, one R⁹ is selected from OR¹⁰, C₁₋₄alkyleneOR¹⁰, OC₁₋₄alkyleneOR¹⁰, and R¹⁰ is selected from C₁₋₂alkyleneC₃₋₁₀cycloalkyl, C₁₋₂alkyleneC₃₋₁₀heterocycloalkyl, C₁₋₂alkyleneOR¹⁴, and C₁₋₂alkyleneNR^{14a}R^{14b} and alkylene, heterocycloalkyl and cycloalkyl groups in R¹⁰ are optionally substituted with one to three of R¹⁵. In an embodiment, one R⁹ is selected from OR¹⁰, C₁₋₄alkyleneOR¹⁰ and OC₁₋₄alkyleneOR¹⁰, and R¹⁰ is selected from C₁₋₂alkyleneC₃₋₆cycloalkyl, C₁₋₂alkyleneC₄₋₇heterocycloalkyl, and C₁₋₂alkyleneNR^{14a}R^{14b} and alkylene, heterocycloalkyl and cycloalkyl groups in R¹⁰ are optionally substituted with one to three of R¹⁵. In an embodiment, the C₄₋₇heterocycloalkyl in R10 is selected from cyclopropyl and cyclobutyl. Therefore, in an embodiment, one R⁹ is selected from OR¹⁰, C₁₋₄alkyleneOR¹⁰ and OC₁₋₄alkyleneOR¹⁰, and R¹⁰ is selected from C₁₋₂alkylenecyclopropyl, C₁₋₂alkylenecyclobutyl, and C₁₋₂alkyleneC₄₋₇heterocycloalkyl. In an embodiment, one R⁹ is selected from OR¹⁰ and C₁₋₄alkyleneOR¹⁰, and R¹⁰ is selected from C₁₋₂alkylenecyclopropyl, C₁₋₂alkylenecyclobutyl, and C₁₋₂alkyleneC₄₋₇heterocycloalkyl. Accordingly, in an embodiment, one R⁹ is selected from OC₁₋₂alkylenecyclopropyl, C₁₋₄alkyleneOC₁₋₂alkylenecyclobutyl, C₁₋₄alkyleneOC₁₋₂alkylenebutyl, C₁₋₂alkyleneC₄₋₇heterocycloalkyl and OC₁₋₂alkyleneC₄₋₇heterocycloalkyl.

In an embodiment, one or two R⁹ are independently selected from C(O)R¹⁰ and CO₂R¹⁰ and each R¹⁰ is selected from H, CH₃, CH₂CH₃, CF₂H, CF₃, CFH₂, CH₂CF₂H, CH₂CF₃, CH(CH₃)₂, CH₂CH(CH₃)₂, and C(CH₃)₃ each of which is optionally substituted with one to three of R¹⁵. In an embodiment, one or two R⁹ is selected from C(O)CH₃, C(O)C(CH₃)₃, CO₂CH₃ and CO₂C(CH₃)₃.

In an embodiment, each R¹⁵ is independently selected from F, Cl, CN, C₁₋₄alkyl and NR^{15a}R^{15b}. In an embodiment, each R¹⁵ is independently selected from F, C₁₋₄alkyl and NR^{15a}R^{15b}. In an embodiment, one of R¹⁵ is F. In an embodiment, one of R¹⁵ is NR^{15a}R^{15b}.

In an embodiment, R^{15a} and R^{15b} are independently selected from H and C₁₋₄alkyl. In an embodiment, R^{15a} and R^{15b} are independently selected from H, CH₃, CH₂CH₃, CF₂H, CF₃, CFH₂, CH₂CF₂H and CH₂CF₃. In an embodiment, R^{15a} and R^{15b} are independently selected from H, CF₃ and CH₃. In an embodiment, R^{15a} and R^{15b} are independently selected from H and CH₃.

In an embodiment, R¹², R^{12a}, and R^{12b} are independently selected from OR¹⁷, C₁₋₄alkyl, C₃₋₁₁cycloalkyl, C₃₋₁₀heterocycloalkyl, C₁₋₄alkyleneC₃₋₁₀cycloalkyl, and C₁₋₄alkyleneC₃₋₁₀heterocycloalkyl. In an embodiment, R¹², R^{12a}, and R^{12b} are independently selected from OR¹⁷, C₁₋₄alkyl, C₃₋₁₁cycloalkyl, C₃₋₁₀heterocycloalkyl, C₁₋₄alkyleneC₃₋₁₀cycloalkyl, and C₁₋₄alkyleneC₃₋₁₀heterocycloalkyl. In an embodiment, one of R¹², R^{12a} and R^{12b} is OR¹⁷ and the two of R¹², R^{12a} and R^{12b} are independently selected from OR¹⁷, C₁₋₄alkyl, C₃₋₁₁cycloalkyl, C₃₋₁₀heterocycloalkyl, C₁₋₄alkyleneC₃₋₁₀cycloalkyl, and C₁₋₄alkyleneC₃₋₁₀heterocycloalkyl. In an embodiment, one of R¹², R^{12a} and R^{12b} is OR¹⁷ and the other two are independently selected from C₁₋₄alkyl, C₃₋₁₁cycloalkyl, C₃₋₁₀heterocycloalkyl, C₁₋₄alkyleneC₃₋₁₀cycloalkyl, and C₁₋₄alkyleneC₃₋₁₀heterocycloalkyl. In an embodiment, one of R¹², R^{12a} and R^{12b} is OR¹⁷ and the other two of R¹², R^{12a} and R^{12b} are selected from OR¹⁷, C₁₋₄alkyl, C₃₋₁₁cycloalkyl, C₃₋₁₀heterocycloalkyl, C₁₋₄alkyleneC₃₋₁₀cycloalkyl, and C₁₋₄alkyleneC₃₋₁₀heterocycloalkyl.

In an embodiment, R¹⁴, R^{14a}, R^{14b} and R¹⁶ are independently selected from H and C₁₋₄alkyl. In an embodiment, R¹⁴, R^{14a}, R^{14b} and R¹⁶ are independently selected from H, CH₃, CH₂CH₃, CF₂H, CF₃, CFH₂, CH₂CF₂H and CH₂CF₃. In an embodiment, R¹⁴, R^{14a}, R^{14b} and R¹⁶ are independently selected from H, CF₃ and CH₃. In an embodiment, R¹⁴, R^{14a}, R^{14b} and R¹⁶ are independently selected from H and CH₃.

In an embodiment, R¹⁷ is selected from H, C₁₋₄alkyl, C₃₋₁₁cycloalkyl, C₃₋₁₀heterocycloalkyl, C₁₋₄alkyleneC₃₋₁₀cycloalkyl, C₁₋₄alkyleneC₃₋₁₀heterocycloalkyl, C₁₋₄alkyleneOR¹⁸, and C₁₋₄alkyleneNR¹⁸R¹⁹. In an embodiment, R¹⁷ is selected from H and C₁₋₄alkyl. In an embodiment, R¹⁷ is H, CH₃, CH₂CH₃, CF₂H, CF₃, CFH₂, CH₂CF₂H and CH₂CF₃. In an embodiment, R¹⁷ is selected from H and C₁₋₄alkyl. In an embodiment, R¹⁷ is H. In an embodiment R¹⁷ is CH₃.

In an embodiment, R¹⁸ and R¹⁹ are independently selected from H and C₁₋₄alkyl.

In an embodiment, Cy¹ is phenyl, pyrrole, or pyridinyl which is substituted with Z-Cy² and one or two of R⁹. In an embodiment, Cy¹ is phenyl, pyrrole, or pyridinyl which is substituted with Z-Cy² and one or two of R⁹, and at least one R⁹ is F. In an embodiment, Cy¹ is phenyl which is substituted with Z-Cy² and one or two of R⁹, and at least one R⁹ is F.

In an embodiment, Cy¹ is phenyl, pyrrole, or pyridinyl which is substituted with one or two of R⁹, or with Z-Cy² and one or two of R⁹. In an embodiment, Cy¹ is phenyl, pyrrole, or pyridinyl which is substituted with one or two of R⁹, Z-Cy² and one or two of R⁹, and at least one R⁹ is F. In an embodiment, Cy¹ is phenyl which is substituted with one or two of R⁹, or with Z-Cy² and one or two of R⁹, and at least one R⁹ is F.

In an embodiment, Cy¹ is phenyl, pyrrole, or pyridinyl which is substituted with one or two of R⁹, or is substituted with Z-Cy² and one to three R⁹ and at least one R⁹ is selected from OR¹⁰, C(O)R¹⁰, CO₂R¹⁰, SR¹⁰, SO₂R¹⁰, S(O)(═NR¹¹)R¹⁰, NR¹¹COR¹⁰, and NR¹¹SO₂R¹⁰. In an embodiment, Cy¹ is phenyl which is substituted with one or two of R⁹, or is substituted with Z-Cy² and one to three R⁹ and at least one R⁹ is selected from OR¹⁰, C(O)R¹⁰, CO₂R¹⁰, SR¹⁰, SO₂R¹⁰, S(O)(═NR¹¹)R¹⁰, NR¹¹COR¹⁰, and NR¹¹SO₂R¹⁰. In an embodiment, Cy¹ is phenyl, pyrrole, or pyridinyl which is substituted with one or two of R⁹, and at least one R⁹ is selected from OR¹⁰, C(O)R¹⁰, CO₂R¹⁰, SR¹⁰, SO₂R¹⁰, S(O)(═NR¹¹)R¹⁰, NR¹¹COR¹⁰, and NR¹¹SO₂R¹⁰. In an embodiment, Cy¹ is phenyl which is substituted with one or two of R⁹, and at least one R⁹ is selected from OR¹⁰, C(O)R¹⁰, CO₂R¹⁰, SR¹⁰, SO₂R¹⁰, S(O)(═NR¹¹)R¹⁰, NR¹¹COR¹⁰, and NR¹¹SO₂R¹⁰. In an embodiment, R¹⁰ is selected from H, CH₃, CH₂CH₃, CH(CH₃)₂, C(CH₃)₃, C₁₋₂alkyleneC₃₋₁₀cycloalkyl, C₁₋₂alkyleneC₃₋₁₀heterocycloalkyl, C₁₋₂alkyleneOR¹⁴, and C₁₋₂alkyleneNR^{14a}R^{14b}. In an embodiment, R¹⁰ is selected from C₁₋₂alkyleneC₃₋₁₀cycloalkyl, and C₁₋₂alkyleneC₃₋₁₀heterocycloalkyl.

In an embodiment, Cy¹ is phenyl, pyrrole, or pyridinyl which is substituted with one to three of R⁹, or is substituted with Z-Cy² and one to three R⁹ and at least one R⁹ is selected from SO₂NR¹⁰R¹¹ and NR¹¹C₁₋₆alkyleneNR¹⁰R¹¹. In an embodiment, Cy¹ is phenyl which is substituted with one to three of R⁹, or is substituted with Z-Cy² and one to three R⁹ and at least one R⁹ is selected from SO₂NR¹⁰R¹¹ and NR¹¹C₁₋₆alkyleneNR¹⁰R¹¹. In an embodiment, Cy¹ is phenyl which is substituted with one to three of R⁹, or is substituted with Z-Cy² and one to three R⁹ and at least one R⁹ is SO₂NR¹⁰R¹¹. In an embodiment, Cy¹ is phenyl which is substituted with one to three of R⁹, or is substituted with Z-Cy² and one to three R⁹ and at least one R⁹ is NR¹¹C₁₋₆alkyleneNR¹⁰R¹¹. In an embodiment each R¹⁰ and R¹¹ are independently selected from H and C₁₋₄alkyl. In an embodiment, Cy¹ is phenyl, pyrrole, or pyridinyl which is substituted with one to three of R⁹, and at least one R⁹ is selected from SO₂NR¹⁰R¹¹ and NR¹¹C₁₋₆alkyleneNR¹⁰R¹¹. In an embodiment, Cy¹ is phenyl which is substituted with one to three of R⁹, and at least one R⁹ is selected from SO₂NR¹⁰R¹¹ and NR¹¹C₁₋₆alkyleneNR¹⁰R¹¹. In an embodiment, Cy¹ is phenyl which is substituted with one to three of R⁹, and at least one R⁹ is SO₂NR¹⁰R¹¹. In an embodiment, Cy¹ is phenyl which is substituted with one to three of R⁹, and at least one R⁹ is NR¹¹C₁₋₆alkyleneNR¹⁰R¹¹. In an embodiment each R¹⁰ and R¹¹ are independently selected from H and C₁₋₄alkyl.

In an embodiment, Cy¹ is phenyl, pyrrole, or pyridinyl which is substituted with one to three of R⁹, or is substituted with Z-Cy² and one to three R⁹ and at least one R⁹ is SiR¹²R^{12a}R^{12b}. In an embodiment, Cy¹ is phenyl which is substituted with one to three of R⁹, or is substituted with Z-Cy² and one to three R⁹ and at least one R⁹ is SiR¹²R^{12a}R^{12b}. In an embodiment, Cy¹ is phenyl, pyrrole, or pyridinyl which is substituted with one to three of R⁹, and at least one R⁹ is SiR¹²R^{12a}R^{12b}. In an embodiment, Cy¹ is phenyl which is substituted with one to three of R⁹, and at least one R⁹ is SiR¹²R^{12a}R^{12b}. In an embodiment, one of R¹², R^{12a} and R^{12b} is OR¹⁷ and the two of R¹², R^{12a} and R^{12b} are independently selected from H, OR¹⁷, C₁₋₄alkyl, C₃₋₁₁cycloalkyl, C₃₋₁₀heterocycloalkyl, C₁₋₄alkyleneC₃₋₁₀cycloalkyl, and C₁₋₄alkyleneC₃₋₁₀heterocycloalkyl.

In an embodiment, Cy¹ is phenyl or pyridinyl which is substituted with one to three of R⁹, or is substituted with Z-Cy² and one to three R⁹ and one or two R⁹ are selected from C₁₋₄alkyleneNR^{10a}R^{11a}, NR^{10a}R^{11a}, SO₂NR¹⁰R¹¹, NR¹¹COR¹⁰, NR¹¹C₁₋₄alkyleneNR¹R¹¹, OR¹⁰, C₁₋₄alkyleneOR¹⁰, C₃₋₇heterocycloalkyl, and C₁₋₄alkyleneC₃₋₇heterocycloalkyl, the latter two groups being optionally substituted with one to four of R¹³ In an embodiment, Cy¹ is phenyl or pyridinyl which is substituted with one to three of R⁹, or is substituted with Z-Cy² and one to three R⁹ and one or two R⁹ are selected from C₁₋₄alkylene NR^{10a}R^{11a}, NR^{10a}R^{11a}, NR¹¹C₁₋₄alkyleneNR^{10a}R^{11a}, OR¹⁰ C₁₋₄alkyleneOR¹⁰, C₃₋₇heterocycloalkyl, and C₁₋₄alkyleneC₃₋₇heterocycloalkyl, the latter two groups being optionally substituted with one to four of R¹³. In an embodiment, Cy¹ is phenyl or pyridinyl which is substituted with one to three of R⁹, or is substituted with Z-Cy² and one to three R⁹ and one or two R⁹ are selected from C₁₋₄alkyleneNR^{10a}R^{11a}. In an embodiment, Cy¹ is phenyl or pyridinyl which is substituted with one to three of R⁹, or is substituted with Z-Cy² and one to three R⁹ and one or two R⁹ are selected from C₁₋₄alkylene NR^{10a}R^{11a}, NR^{10a}R^{11a}, OR¹⁰, C₁₋₄alkyleneOR¹⁰, C₃₋₇heterocycloalkyl, and C₁₋₄alkyleneC₃₋₇heterocycloalkyl, the latter two groups being optionally substituted with one to four of R¹³. In an embodiment, Cy¹ is phenyl or pyridinyl which is substituted with one to three of R⁹, or is substituted with Z-Cy² and one to three R⁹ and one or two R⁹are selected from C₁₋₄alkylene NR^{10a}R^{11a} and OR¹⁰. In an embodiment, Cy¹ is phenyl or pyridinyl which is substituted with one to three of R⁹, or is substituted with Z-Cy² and one to three R⁹ and one or two R⁹ are selected from CH₂N(CH₂CH₃)₂, C(CH₃)₂NH₂, CH₂N(CH₃)₂, CH₂CH₂N(CH₃)₂ and CH₂N(CH₃)₂ and CH₂N(CH₃)₂. In an embodiment, Cy¹ is phenyl or pyridinyl which is substituted with one to three of R⁹, or is substituted with Z-Cy² and one to three R⁹ and one or two R⁹ are selected from C₁₋₄alkyleneC₃₋₇heterocycloalkyl optionally substituted with one to four of R¹³

In an embodiment, Cy¹ is phenyl or pyridinyl which is substituted with one to three of R⁹, or is substituted with Z-Cy² and one of R⁹ is C₁₋₄alkyleneNR^{10a}R^{11a}, R^{10a} is selected from C₁₋₄alkyleneOR¹⁴ and C₁₋₄alkyleneNR^{14a}R^{14b} and R^{11a} is selected from H and C₁₋₄alkyl and all alkylene groups in R^{10a} are optionally substituted with one to three of R¹⁵. In an embodiment, Cy¹ is phenyl or pyridinyl which is substituted with one to three R⁹ or is substituted with Z-Cy² and one to three R⁹ and one R⁹ is C₁₋₄alkyleneN(C₁₋₆alkyleneOR¹⁴)(R^{11a}). In an embodiment, Cy¹ is phenyl or pyridinyl which is substituted with one to three of R⁹, or is substituted with Z-Cy² and one to three R⁹ and one or two R⁹ are selected from C₁₋₄alkyleneNR^{10a}R^{11a} and R¹⁰a is selected from C₃₋₇heterocycloalkyl and C₁₋₄alkyleneC₃₋₇heterocycloalkyl and R^{11a} is selected from H and C₁₋₄alkyl. In an embodiment, Cy¹ is phenyl or pyridinyl which is substituted with one to three of R⁹, or is substituted with Z-Cy² and one to three R⁹ and one or two R⁹are selected from C₁₋₄alkyleneNR^{10a}R^{11a} and R^{10a} is selected from C₃₋₇heterocycloalkyl and C₁₋₄alkyleneC₃₋₇heterocycloalkyl and R^{11a} is selected from H and C₁₋₄alkyl wherein the C₃₋₇heterocycloalkyl in the C₁₋₄alkyleneC₃₋₇heterocycloalkyl is selected from azetidinyl, pyrrolidinyl, pyrrolidin-2-onyl, piperidinyl, piperazinyl and morpholinyl.

In an embodiment, Cy¹ is phenyl, pyrrole, or pyridinyl which is substituted with one to three of R⁹, or is substituted with Z-Cy² and one to three R⁹ and a R⁹ is pyrrolidinyl optionally substituted with one or more of R¹³. In an embodiment, Cy¹ is phenyl which is substituted with one to three of R⁹; or is substituted with Z-Cy² and one to three R⁹ and a R⁹ is pyrrolidinyl, optionally substituted with one or more of R¹³. In an embodiment, Cy¹ is phenyl, pyrrole, or pyridinyl which is substituted with one to three of R⁹, and a R⁹ is pyrrolidinyl optionally substituted with one or more of R¹³. In an embodiment, Cy¹ is phenyl which is substituted with one to three of R⁹; and a R⁹ is pyrrolidinyl, optionally substituted with one or more of R¹³

In an embodiment, Cy¹ is phenyl, pyrrole, or pyridinyl which is substituted with one to three of R⁹, or is substituted with Z-Cy² and one to three R⁹ and a R⁹ is C₁₋₄alkylenepyrrolidinyl optionally substituted with one or two of R¹³. In an embodiment, Cy¹ is phenyl which is substituted with one to three of R⁹, or is substituted with Z-Cy² and one to three R⁹ and a R⁹ is C₁₋₄alkylenepyrrolidinyl optionally substituted with one or two of R¹³. In an embodiment, Cy¹ is a bicyclic C₉₋₁₁heterocycloalkyl or spirofused tricyclic C₉₋₁₆ heterocycloalkyl which is unsubstituted. In an embodiment, Cy¹ is phenyl, pyrrole, or pyridinyl which is substituted with one to three of R⁹, and a R⁹ is C₁₋₄alkylenepyrrolidinyl optionally substituted with one or two of R¹³. In an embodiment, Cy¹ is phenyl which is substituted with one to three of R⁹, and a R⁹ is C₁₋₄alkylenepyrrolidinyl optionally substituted with one or two of R¹³. In an embodiment, Cy¹ is a bicyclic C₉₋₁₁heterocycloalkyl or spirofused tricyclic C₉₋₁₆heterocycloalkyl which is unsubstituted.

In an embodiment, Cy¹ is a bicyclic C₉₋₁₁aryl or tricyclic C₁₁₋₁₆aryl group is fused and/or spiro fused, to one or two heterocycloalkyl groups, and Cy¹ is unsubstituted. In an embodiment, Cy¹ is a bicyclic C₉₋₁₁aryl or tricyclic C₁₁₋₁₆aryl wherein the aryl group is fused and/or spiro fused, to one or two heterocycloalkyl groups, and Cy¹ is substituted with one to three of R⁹, or is substituted with Z-Cy² and one to three R⁹ and each R⁹ is selected from F, Cl, CN, NO₂, C₁₋₄alkyl, OR¹⁰, C(O)R¹⁰, CO₂R¹⁰, SR¹⁰, SO₂R¹⁰, C₁₋₄alkyleneNR^{10a}R^{11a}, NR^{10a}R^{11a} and C₁₋₆alkyleneOR¹⁰. In an embodiment, Cy¹ is a bicyclic C₉₋₁₁aryl wherein the aryl group is fused to a heterocycloalkyl group, and Cy¹ is substituted with one to three of R⁹, and each R⁹ is selected from F, Cl, CN, NO₂, C₁₋₄alkyl, OR¹⁰, C₁₋₄alkyleneNR^{10a}R^{11a}, NR^{10a}R^{11a} and C₁₋₆alkyleneOR¹⁰. In an embodiment, Cy¹ is a tricyclic C₁₁₋₁₆aryl which is unsubstituted or is substituted with one to three of R⁹, or is substituted with Z-Cy² and one to three R⁹ and each R⁹ is selected from F, C₁₋₄alkyl, OR¹⁰, C(O)R¹⁰, CO₂R¹⁰, C₁₋₄alkyleneNR^{10a}R^{11a}, NR^{10a}R^{11a}, and C₁₋₆alkyleneOR¹⁰. In an embodiment, Cy¹ is a tricyclic C₉₋₁₆aryl wherein the aryl group is fused and/or spirofused to two heterocycloalkyl groups, and Cy¹ is substituted with one to two of R⁹, or is substituted with Z-Cy² and one to three R⁹ and a R⁹ is selected from OR¹⁰, C(O)R¹⁰, and CO₂R¹⁰. In an embodiment, R¹⁰ is selected from H, CH₃, CH₂CH₃, CF₂H, CF₃, CFH₂, CH₂CF₂H, CH₂CF₃ and C(CH₃)₃.

In an embodiment, Cy¹ is a benzo-fused bicyclic C₉₋₁₁aryl wherein the aryl group is a phenyl and is fused to a heterocycloalkyl group, or tricyclic C₁₁₋₁₆aryl wherein the aryl group is phenyl and is fused and/or spiro fused to one or two heterocycloalkyl groups, all of which are substituted with one to three of R⁹, or is substituted with Z-Cy² and one to three R⁹ and R⁹is selected from CH₃, CF₂H, CFH₂, CH₂CF₂H, CH₂CH₂F₂H, CH₂CH₂CH₂F₂H, OCH₃, OCF₂H, OCFH₂, OCH₂CF₂H, OCH₂CH₂F₂H, OCH₂CH₂CH₂F₂H, C₁₋₄alkyleneNR^{10a}R^{11a} and NR^{10a}R^{11a}

In an embodiment, Z is absent. It is appreciated by a person skilled in the art that when Z is absent, Cy² is covalently attached to Cy¹ by a direct bond.

In an embodiment, Z is selected from C₁₋₄alkylene, O, C(O), CO₂, SO₂, S(O)(═NHR^{11b}) and NR^{11b}. In an embodiment, Z is selected from C₁₋₄alkylene, O, C(O), and SO₂. In an embodiment, Z is O. In an embodiment, Z is SO₂.

In an embodiment, Z is selected from C₁₋₆alkyleneO, C₁₋₆alkyleneC(O), C₁₋₆alkyleneCO₂, C₁₋₆alkyleneS, C₁₋₆alkyleneS(O), C₁₋₆alkyleneSO₂, C₁₋₆alkyleneS(O)(═NR^{11b}) C₁₋₆alkyleneNR^{11b}, OC₁₋₆alkylene, C(O)C₁₋₆alkylene, CO₂C₁₋₆alkylene, SC₁₋₆alkylene, S(O)C₁₋₆alkylene, SO₂C₁₋₆alkylene, S(O)(═NR^{11b})C₁₋₆alkylene and NR^{11b}C₁₋₆alkylene. In an embodiment, Z is selected from C₁₋₄alkyleneO, C₁₋₄alkyleneC(O), C₁₋₆alkyleneCO₂, C₁₋₄alkyleneS, C₁₋₄alkyleneS(O), C₁₋₄alkyleneSO₂, C₁₋₄alkyleneS(O)(═NR^{11b}), C₁₋₄alkyleneNR^{11b}, OC₁₋₄alkylene, C(O)C₁₋₄alkylene, CO₂C₁₋₄alkylene, SC₁₋₄alkylene, S(O)C₁₋₄alkylene, SO₂C₁₋₄alkylene, S(O)(═NR^{11b})C₁₋₄alkylene and NR^{11b}C₁₋₆alkylene. In an embodiment, Z is selected from C₁₋₄alkyleneO, C₁₋₄alkyleneC(O), C₁₋₆alkyleneCO₂, C₁₋₄alkyleneS, C₁₋₄alkyleneS(O), C₁₋₄alkyleneSO₂, C₁₋₄alkyleneNR^{l}1b, OC₁₋₄alkylene, C(O)C₁₋₄alkylene, CO₂C₁₋₄alkylene, SC₁₋₄alkylene, S(O)C₁₋₄alkylene, SO₂C₁₋₄alkylene, and NR^{11b}C₁₋₆alkylene. In an embodiment, Z is selected from C₁₋₄alkyleneO, C₁₋₄alkyleneC(O), OC₁₋₄alkylene and C(O)C₁₋₄alkylene. In an embodiment, Z is selected from C₁₋₄alkyleneO, C₁₋₄alkyleneC(O), C₁₋₆alkyleneCO₂, C₁₋₄alkyleneS, C₁₋₄alkyleneS(O), C₁₋₄alkyleneSO₂, C₁₋₄alkyleneNR^{11b}, OC₁₋₄alkylene, C(O)C₁₋₄alkylene, CO₂C₁₋₄alkylene, SC₁₋₄alkylene, S(O)C₁₋₄alkylene, SO₂C₁₋₄alkylene, and NR^{11b}C₁₋₆alkylene. In an embodiment, Z is selected from OC₁₋₄alkylene and C(O)C₁₋₄alkylene. In an embodiment, Z is OC₁₋₄alkylene.

In an embodiment, R^{11b} is selected from H and C₁₋₄alkyl. In an embodiment, R^{11b} is selected from H, CH₃, CH₂CH₃, CF₂H, CF₃, CFH₂, CH₂CF₂H, CH₂CH₂CF₂H, CH₂CH₂CH₂CF₂H, and CH₂CF₃. In an embodiment, R^{11b} is selected from H and C₁₋₄alkyl. In an embodiment, R^{11b} is selected from H, CH₃, CH₂CH₃, CF₂H, CF₃, CFH₂, CH₂CF₂H and CH₂CF₃. In an embodiment, R^{11b} is selected from CH₂CH₂CF₂H and CH₂CH₂CH₂CF₂H. In an embodiment, R^{11b} is selected from H, CF₃ and CH₃. In an embodiment, R^{11b} is selected from H and CH₃.

In an embodiment, Cy² is C₃₋₁₁cycloalkyl and Cy² is unsubstituted or substituted with one or more of R²⁰. In an embodiment, Cy² is selected from cyclopropyl, cyclobutyl, cyclopentyl, cyclohexyl, and cycloheptyl. In an embodiment, Cy² is cyclopropyl.

In an embodiment, Cy² is a monocyclic C₃₋₇heterocycloalkyl and Cy² is unsubstituted or substituted with one or more of R²⁰. In an embodiment, Cy² is selected from azetidinyl, oxetanyl, tetrahydrofuranyl, tetrahydrothiophenyl, pyrrolidinyl, dihydropyrrolyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, dioxidothiomorpholino, tetrahydropyridinyl, dihydropyridinyl, dihydropyranyl, thianyl, piperidinyl, piperazinyl, dihydropyranyl, tetrahydropyranyl, thiomorpholinyl, morpholinyl, dioxanyl, azepanyl, diazepanyl, oxepanyl, and thiepanyl and Cy² is unsubstituted or substituted with one to four of R²⁰. In an embodiment, Cy² is selected from azetidinyl, 5,6-dihydro-1,2,4-triazinyl, 5,6-dihydro-4H-1,2,4-oxadiazinyl, dioxidothiomorpholino, thiomorpholinyl, tetrahydrofuranyl, tetrahydropyranyl, diazepanyl, pyrrolidinyl, piperidinyl, piperazinyl, and morpholinyl, and Cy² is unsubstituted or substituted with one to three of R²⁰. In an embodiment, Cy² is selected from tetrahydrofuranyl, dihydropyranyl, tetrahydropyranyl, pyrrolidinyl, piperidinyl, piperazinyl and morpholinyl, which is substituted with one to three of R²⁰. In an embodiment, Cy² is selected from pyrrolidinyl, piperidinyl, piperazinyl, and morpholinyl, and Cy² is unsubstituted or substituted with one or two of R²⁰. In an embodiment, Cy² is a monocyclic C₃₋₇heterocycloalkyl and Cy² is unsubstituted or substituted with one or more of R²⁰. In an embodiment, Cy² is selected from azetidinyl, oxetanyl, tetrahydrofuranyl, tetrahydrothiophenyl, pyrrolidinyl, dihydropyrrolyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, dioxidothiomorpholino, tetrahydropyridinyl, dihydropyridinyl, dihydropyranyl, thianyl, piperidinyl, piperazinyl, tetrahydropyranyl, thiomorpholinyl, morpholinyl, dioxanyl, azepanyl, diazepanyl, oxepanyl, and thiepanyl and Cy² is unsubstituted or substituted with one to four of R²⁰. In an embodiment, Cy² is selected from azetidinyl, 5,6-dihydro-1,2,4-triazinyl, 5,6-dihydro-4H-1,2,4-oxadiazinyl, dioxidothiomorpholino, thiomorpholinyl, tetrahydrofuranyl, tetrahydropyranyl, diazepanyl, pyrrolidinyl, piperidinyl, piperazinyl, and morpholinyl, and Cy² is unsubstituted or substituted with one to three of R²⁰. In an embodiment, Cy² is selected from tetrahydrofuranyl, tetrahydropyranyl, pyrrolidinyl, piperidinyl, piperazinyl and morpholinyl, which is substituted with one to three of R²⁰. In an embodiment, Cy² is selected from pyrrolidinyl, piperidinyl, piperazinyl, and morpholinyl, and Cy² is unsubstituted or substituted with one or two of R²⁰. In an embodiment, Cy² is dihydropyranyl. In an embodiment, Cy² is selected from piperidinyl, piperazinyl, and morpholinyl, and Cy² is unsubstituted or substituted with one or two of R²⁰. In an embodiment, Cy² is piperazinyl and Cy² is unsubstituted or substituted with one or two of R²⁰. In an embodiment, Cy² is selected from tetrahydrofuranyl, dihydropyranyl, morpholinyl, and tetrahydropyranyl, which is substituted with one to three of R²⁰. In an embodiment, Cy² is tetrahydropyranyl, which is substituted with one to three of R²⁰.

In an embodiment, Cy² is selected from

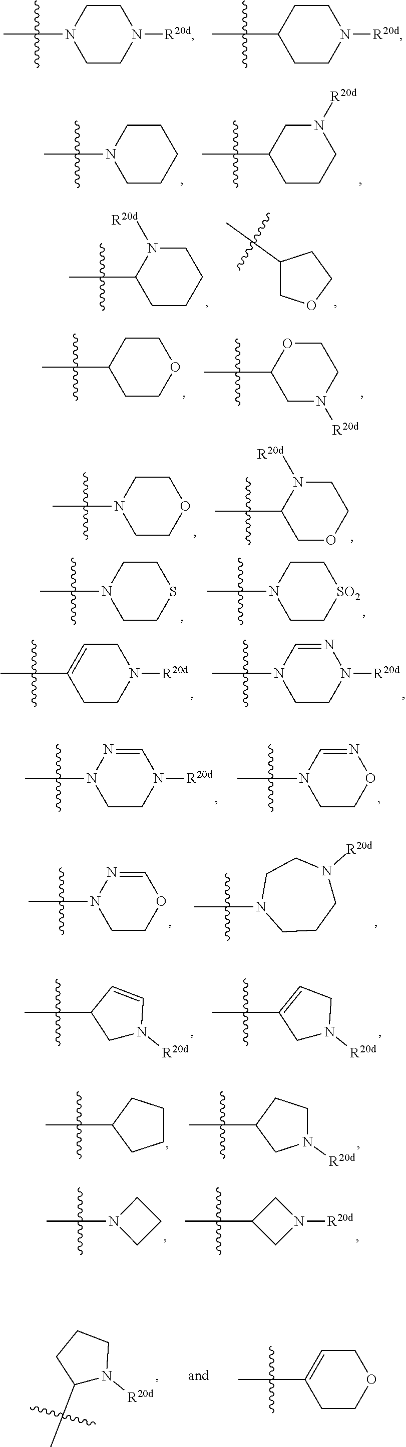

which is unsubstituted or substituted with one to four of R²⁰, and
wherein
R^{20d} is selected from H and R²⁰; and
[symbol] indicates a point of covalent attachment to Z.

In an embodiment, Cy² is selected from

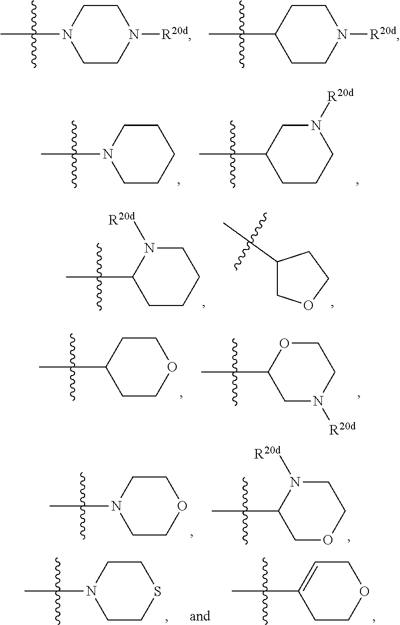

which is unsubstituted or substituted with one to four of R²⁰, and
wherein
R^{20d} is selected from H and R²⁰; and
[symbol] indicates a point of covalent attachment to Z.

In an embodiment, Cy² is selected from

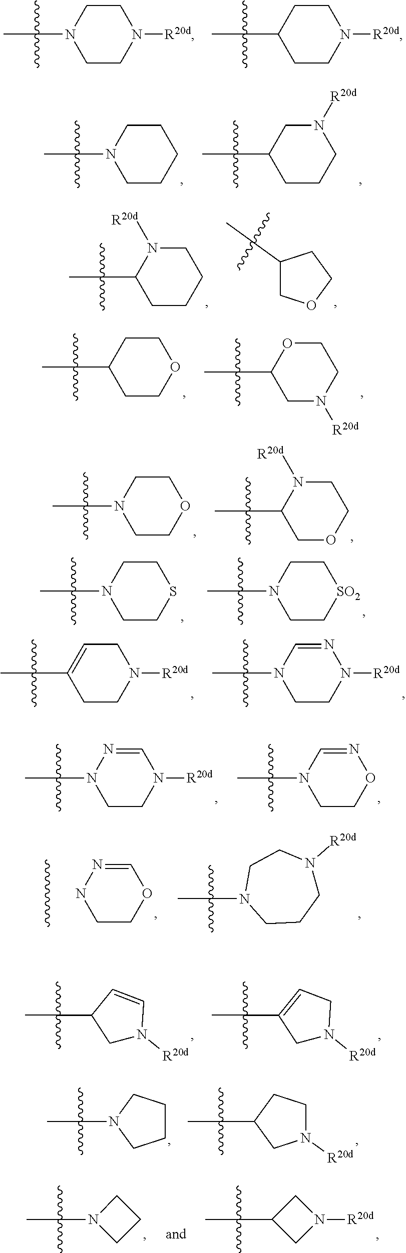

which is unsubstituted or substituted with one to four of R²⁰, and
wherein
R^{20d} is selected from H and R²⁰, and
[symbol] indicates a point of covalent attachment to Z.

In an embodiment, Cy² is a bicyclic heterocycle and Cy² is unsubstituted or substituted with one to four of R²⁰.In an embodiment, Cy² is a bridged bicyclic heterocycle, fused bicyclic heterocycle or a spirofused bicyclic heterocycle and Cy² is unsubstituted or substituted with one to four of R²⁰. In an embodiment, Cy² is a bridged bicyclic heterocycle and Cy² is unsubstituted or substituted with one to four of R²⁰.

In an embodiment, Cy² is a fused bicyclic heterocycle which is unsubstituted or substituted with one to four of R²⁰. In an embodiment, Cy² is a C₆-C₁₀ saturated bicyclic ring in which one or two of the ring carbon atoms is replaced with N, NH, or NR²⁰, depending on the valency requirements of the N, which is unsubstituted or further substituted with one or four of R²⁰. In an embodiment, Cy² is azabicyclohexanyl, diazabicycloheptanyl, or diazabicyclooctanyl and Cy² is unsubstituted or substituted with one to three of R²⁰. In an embodiment, Cy² is bridged azabicyclohexanyl, bridged diazabicycloheptanyl or bridged diazabicyclooctanyl. unsubstituted or substituted with one to three of R²⁰. In an embodiment, Cy² is selected from the following structures

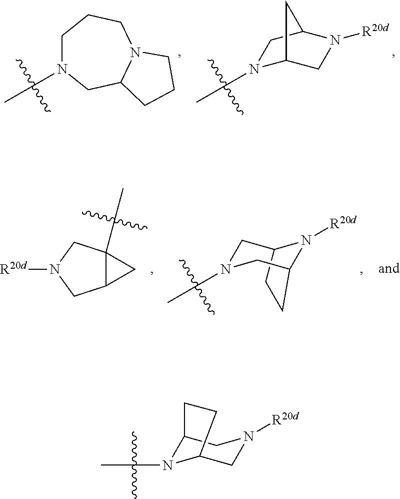

which is unsubstituted or substituted with one to four of R²⁰, and
wherein
R^{20d} is selected from H and R²⁰, and
[symbol] indicates a point of covalent attachment to Cy¹.

In an embodiment, Cy² is selected from the following structures:

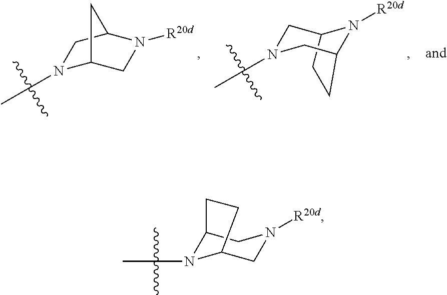

which is unsubstituted or substituted with one to four of R²⁰, and
wherein
R^{20d} is selected from H and R²⁰, and
[symbol] indicates a point of covalent attachment to Z.

In an embodiment, Cy² is

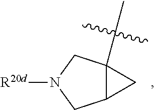

wherein R^{20d} is selected from H and R²⁰, and [symbol] indicates a point of covalent attachment to Z.

In an embodiment, Cy² is selected from tetrahydrofuropyrrolyl, hexapyrazinooxazinyl, hexahydropyrrolopyrazinyl, and hexahydropyrrolodiazepiny and Cy² is unsubstituted or substituted with one to four of R²⁰.

In an embodiment, Cy² is selected from

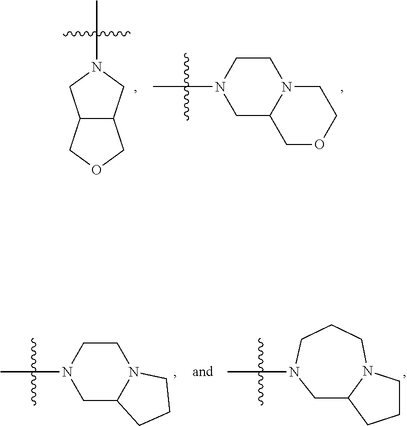 [SMILES: *N1CC2COCC2C1 ; *N1CCN2CCOCC2C1 ; *N1CCN2CCCC2C1 ; *N1CCCN2CCCC2C1]

which is unsubstituted or substituted with one to four of R²⁰; and
wherein [symbol] indicates a point of covalent attachment to Z.

In an embodiment, Cy² is spirofused bicyclic heterocycle and Cy² is unsubstituted or substituted with one to four of R²⁰.In an embodiment, Cy² is selected from

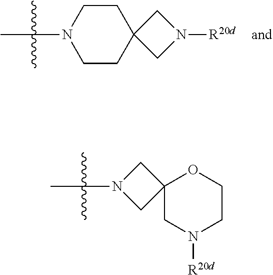

which is unsubstituted or substituted with one to four of R²⁰; and
wherein R^{20d} is selected from H and R²⁰, and [symbol] indicates a point of covalent attachment to Z.

It is appreciated by a person skilled in the art that when Z is absent, then [symbol] in the structures of Cy² indicates a point of covalent attachment to Cy¹.

In an embodiment, Cy² is unsubstituted. In an embodiment, Cy² is substituted with one to three of R²⁰. In an embodiment, Cy² is substituted with one or two of R²⁰. In an embodiment, Cy² is substituted with one R²⁰.

In an embodiment, each R²⁰ is independently selected from F, Cl, ═O, CN, OH, C₁₋₄alkyl, C₂₋₄alkenyl, C₂₋₄alkynyl, C₃₋₁₀cycloalkyl, C₃₋₁₀heterocycloalkyl, C₆₋₁₁aryl, C₅₋₁₄heteroaryl, C₁₋₄alkyleneC₃₋₁₀cycloalkyl, C₁₋₄alkyleneC₃₋₁₀heterocycloalkyl, C₁₋₄alkyleneC₆₋₁₁aryl, C₁₋₄alkyleneC₅₋₁₄heteroaryl, OC₁₋₄alkyl, OC₂₋₄alkenyl, OC₂₋₄alkynyl, C₁₋₄alkyleneOR²¹, OC₁₋₄alkyleneOR²¹, C₁₋₄alkyleneNR²²R²³, OC₁₋₄alkyleneNR²²R²³, SC₁₋₄alkyl, SC₂₋₄alkenyl, SC₂₋₄alkynyl, C(O)C₁₋₄alkyl, C(O)C₂₋₄alkenyl, C(O)C₂₋₄alkynyl, C(O)C₃₋₁₀cycloalkyl, C(O)C₃₋₁₀heterocycloalkyl, C(O)C₆₋₁₁aryl, C(O)C₅₋₁₄heteroaryl, C(O)C₁₋₄alkyleneC₃₋₁₀cycloalkyl, C(O)C₁₋₄alkyleneC₃₋₁₀heterocycloalkyl, C(O)C₁₋₆alkyleneC₆₋₁₁aryl, C(O)C₁₋₆alkyleneC₅₋₁₄heteroaryl, C(O)C₁₋₄alkylenylOR²¹, C(O)C₁₋₄alkyleneNR²²R²³, C(O)C₁₋₄alkyleneOC₁₋₄ alkyleneNR²²R²³, C(O)NR²²R²³, CO₂C₁₋₆alkyl, CO₂C₂₋₄alkenyl, CO₂C₂₋₄alkynyl, CO₂C₁₋₄alkyleneOR²¹, CO₂C₁₋₄alkyleneOC₁₋₄alkyleneNR²²R²³, NR²²R²³, NR²⁴C₃₋₁₀cycloalkyl, NR²⁴C₃₋₁₀heterocycloalkyl, NR²⁴C₆₋₁₁aryl, NR²⁴C₅₋₁₄heteroaryl, NR²⁴C₁₋₄alkyleneC₃₋₁₀cycloalkyl, NR²⁴C₁₋₄alkyleneC₃₋₁₀heterocycloalkyl, NR²⁴C₁₋₆alkyleneC₆₋₁₁aryl, NR²⁴C₁₋₆alkyleneC₅₋₁₄heteroaryl NR²⁴C₁₋₄alkyleneOR²¹, NR²⁴SO₂C₁₋₄alkyl, SO₂C₁₋₆alkyl, SO₂C₂₋₄alkenyl, SO₂C₂₋₄alkynyl, and SO₂NR²²R²³, and alkyl, alkenyl, alkynyl, alkylene, aryl, heteroaryl, heterocycloalkyl, and cycloalkyl groups of R²⁰ are optionally substituted with one to four of R²⁵. In an embodiment, each R²⁰ is independently selected from F, Cl, ═O, CN, OH, C₁₋₄alkyl, C₂₋₄alkenyl, C₂₋₄alkynyl, C₃₋₁₀cycloalkyl, C₃₋₁₀heterocycloalkyl, C₁₋₄alkyleneC₃₋₁₀cycloalkyl, C₁₋₄alkyleneC₃₋₁₀heterocycloalkyl, OC₁₋₄alkyl, OC₂₋₄alkenyl, OC₂₋₄alkynyl, C₁₋₄alkyleneOR²¹, OC₁₋₄alkyleneOR²¹, C₁₋₄alkyleneNR²²R²³, OC₁₋₄alkyleneNR²²R²³, SC₁₋₄alkyl, SC₂₋₄alkenyl, SC₂₋₄alkynyl, C(O)C₁₋₄alkyl, C(O)C₂₋₄alkenyl, C(O)C₂₋₄alkynyl, C(O)C₃₋₁₀cycloalkyl, C(O)C₃₋₁₀heterocycloalkyl, C(O)C₁₋₄alkyleneC₃₋₁₀cycloalkyl, C(O)C₁₋₄alkyleneC₃₋₁₀heterocycloalkyl, C(O)C₁₋₄alkylenylOR²¹, C(O)C₁₋₄alkyleneNR²²R²³, C(O)C₁₋₄alkyleneOC₁₋₄alkyleneNR²²R²³, C(O)NR²²R²³, CO₂C₁₋₆alkyl, CO₂C₂₋₄alkenyl, CO₂C₂₋₄alkynyl, CO₂C₁₋₄alkyleneOR²¹, CO₂C₁₋₄alkyleneOC₁₋₄alkyleneNR²²R²³, NR²²R²³, NR²⁴C₃₋₁₀cycloalkyl, NR²⁴C₃₋₁₀heterocycloalkyl, NR²⁴C₁₋₄alkyleneC₃₋₁₀cycloalkyl, NR²⁴C₁₋₄alkyleneC₃₋₁₀heterocycloalkyl, NR²⁴C₁₋₄alkyleneOR²¹, NR²⁴SO₂C₁₋₄alkyl, SO₂C₁₋₆alkyl, SO₂C₂₋₄alkenyl, SO₂C₂₋₄alkynyl, and SO₂NR²²R²³, and alkyl, alkenyl, alkynyl, alkylene, and cycloalkyl groups of R²⁰ are optionally substituted with one to four of R²⁵.

In an embodiment, each R²⁰ is independently selected from F, Cl, ═O, CN, OH, NO₂, C₁₋₄alkyl, C₃₋₁₀cycloalkyl, C₃₋₁₀heterocycloalkyl, C₆₋₁₁aryl, C₅₋₁₄heteroaryl, C₁₋₄alkyleneC₃₋₁₀cycloalkyl, C₁₋₄alkyleneC₃₋₁₀heterocycloalkyl, C₁₋₄alkyleneC₆₋₁₁aryl, C₁₋₄alkyleneC₅₋₁₄heteroaryl, OC₁₋₄alkyl, C₁₋₄alkyleneOR²¹, OC₁₋₄alkyleneOR²¹, C₁₋₄alkyleneNR²²R²³, OC₁₋₄alkyleneNR²²R²³, SC₁₋₄alkyl, C(O)C₁₋₄alkyl, C(O)C₃₋₁₀cycloalkyl, C(O) C₆₋₁₁aryl, C(O)C₅₋₁₄heteroaryl, C(O)C₃₋₁₀heterocycloalkyl, C(O)C₁₋₄alkyleneC₃₋₁₀cycloalkyl, C(O)C₁₋₄alkyleneC₃₋₁₀heterocycloalkyl, C(O)C₁₋₄alkyleneC₆₋₁₁aryl, C(O)C₁₋₄alkyleneC₅₋₁₄ heteroaryl, C(O)C₁₋₄alkyleneOR²¹, C(O)C₁₋₄alkyleneNR²²R²³, C(O)C₁₋₄alkyleneOC₁₋₄ alkyleneNR²²R²³, C(O)NR²²R²³, CO₂C₁₋₆alkyl, CO₂C₁₋₄alkyleneOR²¹, CO₂C₁₋₄alkyleneOC₁₋₄alkyleneNR²²R²³, NR²²R²³, NR²⁴C₃₋₁₀cycloalkyl, NR²⁴C₃₋₁₀heterocycloalkyl, NR²⁴C₁₋₄alkyleneC₃₋₁₀cycloalkyl, NR²⁴C₆₋₁₁aryl, NR²⁴C₅₋₁₀heteroaryl, NR²⁴C₁₋₄alkyleneC₃₋₁₀heterocycloalkyl, NR²⁴C₁₋₄alkyleneC₆₋₁₁aryl, NR²⁴C₁₋₄alkyleneC₅₋₁₀heteroaryl, NR²⁴C₁₋₄alkyleneOR²¹, NR²⁴SO₂C₁₋₄alkyl, SO₂C₁₋₆alkyl, and SO₂NR²²R²³, and alkyl, alkenyl, alkynyl, alkylene, aryl, heteroaryl, heterocycloalkyl and cycloalkyl groups of R²⁰ are optionally substituted with one to three of R²⁵. In an embodiment, each R²⁰ is independently selected from F, Cl, ═O, CN, OH, NO₂, C₁₋₄alkyl, C₃₋₁₀cycloalkyl, C₃₋₁₀heterocycloalkyl, C₁₋₄alkyleneC₃₋₁₀cycloalkyl, C₁₋₄alkyleneC₃₋₁₀heterocycloalkyl, OC₁₋₄alkyl, C₁₋₄alkyleneOR²¹, OC₁₋₄alkyleneOR²¹, C₁₋₄alkyleneNR²²R²³, OC₁₋₄alkyleneNR²²R²³, SC₁₋₄alkyl, C(O)C₁₋₄alkyl, C(O)C₃₋₁₀cycloalkyl, C(O)C₃₋₁₀heterocycloalkyl, C(O)C₁₋₄alkyleneC₃₋₁₀cycloalkyl, C(O)C₁₋₄alkyleneC₃₋₁₀heterocycloalkyl, C(O)C₁₋₄alkyleneOR²¹, C(O)C₁₋₄alkyleneNR²²R²³, C(O)C₁₋₄alkyleneOC₁₋₄alkyleneNR²²R²³, C(O)NR²²R²³, CO₂C₁₋₆alkyl, CO₂C₁₋₄alkyleneOR²¹, CO₂C₁₋₄alkyleneOC₁₋₄alkyleneNR²²R²³, NR²²R²³, NR²⁴C₃₋₁₀cycloalkyl, NR²⁴C₃₋₁₀heterocycloalkyl, NR²⁴C₁₋₄alkyleneC₃₋₁₀cycloalkyl, NR²⁴C₁₋₄alkyleneC₃₋₁₀heterocycloalkyl, NR²⁴C₁₋₄alkyleneOR²¹, NR²⁴SO₂C₁₋₄alkyl, SO₂C₁₋₆alkyl, and SO₂NR²²R²³, and alkyl, alkenyl, alkynyl, alkylene, and cycloalkyl groups of R²⁰ are optionally substituted with one to three of R²⁵.

In an embodiment, each R²⁰ is independently selected from F, Cl, ═O, CN, OH, NO₂, C₁₋₄alkyl, C₃₋₁₀cycloalkyl, C₃₋₁₀heterocycloalkyl, C₆₋₁₁aryl, C₅₋₁₄heteroaryl, C₁₋₂alkyleneC₃₋₁₀cycloalkyl, C₁₋₂alkyleneC₃₋₁₀heterocycloalkyl, C₁₋₂alkyleneC₆₋₁₁aryl, C₁₋₂alkyleneC₅₋₁₄heteroaryl, OC₁₋₄alkyl, C₁₋₄alkyleneOR²¹, C(O)C₁₋₄alkyl, C(O)C₃₋₁₀cycloalkyl, C(O)C₆₋₁₁aryl, C(O)C₅₋₁₄heteroaryl, C(O)C₃₋₁₀heterocycloalkyl, C(O)C₁₋₄alkyleneC₃₋₁₀cycloalkyl, C(O)C₁₋₄alkyleneC₃₋₁₀heterocycloalkyl, C(O)C₁₋₄alkyleneC₆₋₁₁aryl, C(O)C₁₋₄alkyleneC₅₋₁₄heteroaryl, C(O)C₁₋₄alkyleneOR²¹, C(O)C₁₋₄alkyleneNR²²R²³, C(O)C₁₋₄alkyleneOC₁₋₄alkyleneNR²²R²³, CO₂C₁₋₆alkyl, CO₂C₁₋₄alkyleneOR²¹, NR²²R²³, NR²⁴C₃₋₁₀cycloalkyl, NR²⁴C₃₋₁₀heterocycloalkyl, NR²⁴C₆₋₁₁aryl, NR²⁴C₅₋₁₀heteroaryl, NR²⁴C₁₋₂alkyleneC₃₋₁₀cycloalkyl, NR²⁴C₁₋₂alkyleneC₃₋₁₀heterocycloalkyl, NR²⁴C₁₋₄alkyleneC₆₋₁₁aryl, NR²⁴C₁₋₄alkyleneC₅₋₁₀heteroaryl, NR²⁴C₁₋₄alkyleneOR²¹, NR²⁴SO₂C₁₋₄alkyl, and SO₂C₁₋₆alkyl, and alkyl, alkenyl, alkynyl, alkylene, aryl, heteroaryl, heterocycloalkyl and cycloalkyl groups of R²⁰ are optionally substituted with one to three of R²⁵. In an embodiment, each R²⁰ is independently selected from F, Cl, ═O, CN, OH, NO₂, C₁₋₄alkyl, C₃₋₁₀cycloalkyl, C₃₋₁₀heterocycloalkyl, C₁₋₂alkyleneC₃₋₁₀cycloalkyl, C₁₋₂alkyleneC₃₋₁₀heterocycloalkyl, OC₁₋₄alkyl, C₁₋₄alkyleneOR²¹, C(O)C₁₋₄alkyl, C(O)C₃₋₁₀cycloalkyl, C(O)C₃₋₁₀heterocycloalkyl, C(O)C₁₋₄alkyleneC₃₋₁₀cycloalkyl, C(O)C₁₋₄alkyleneC₃₋₁₀heterocycloalkyl, C(O)C₁₋₄alkyleneOR²¹, C(O)C₁₋₄alkyleneNR²²R²³, C(O)C₁₋₄alkyleneOC₁₋₄alkyleneNR²²R²³, CO₂C₁₋₆alkyl, CO₂C₁₋₄alkyleneOR²¹, NR²²R²³, NR²⁴C₃₋₁₀cycloalkyl, NR²⁴C₃₋₁₀heterocycloalkyl, NR²⁴C₁₋₂alkyleneC₃₋₁₀cycloalkyl, NR²⁴C₁₋₂alkyleneC₃₋₁₀heterocycloalkyl, NR²⁴C₁₋₄alkyleneOR²¹, NR²⁴SO₂C₁₋₄alkyl, and SO₂C₁₋₆alkyl, and alkyl, alkenyl, alkynyl, alkylene, and cycloalkyl groups of R²⁰ are optionally substituted with one to three of R²⁵.

In an embodiment, each R²⁰ is independently selected from F, Cl, ═O, CN, OH, CH₃, CH₂CH₃, CH(CH₃)₂, C(CH₃)₃, CF₂H, CF₃, CFH₂, CH₂CFH₂, CH₂CF₂H, CH₂CF₃, CH₂CH₂CF₃, CH₂CH₂CF₂H, CH₂CH₂CH₂CF₂H, CH₂CH₂CH₂CF₃, C₃₋₆cycloalkyl, C₃₋₆heterocycloalkyl, phenyl, C₅₋₁₄heteroaryl, C₁₋₄alkyleneC₃₋₆cycloalkyl, C₁₋₄alkyleneC₃₋₆heterocycloalkyl, C₁₋₄alkylenephenyl, C₁₋₄alkyleneC₅₋₁₄heteroaryl, OC₁₋₄alkyl, C₁₋₄alkyleneOH, C₁₋₄alkyleneOCH₃, C(O)C₁₋₄alkyl, C(O)C₃₋₆cycloalkyl, C(O)C₃₋₆heterocycloalkyl, C(O)C₆₋₁₁aryl, C(O)C₅₋₁₄heteroaryl, C(O)C₁₋₂alkyleneC₃₋₆cycloalkyl, C(O)C₁₋₂alkyleneC₃₋₁₀heterocycloalkyl, C(O)C₁₋₄alkyleneC₆₋₁₁aryl, C(O)C₁₋₄alkyleneC₅₋₁₄heteroaryl, C(O)C₁₋₄alkyleneOCH₃, C(O)C₁₋₄alkyleneOH, C(O)C₁₋₄alkyleneNH₂, C(O)C₁₋₄alkyleneN(CH₃)₂, C(O)C₁₋₄alkyleneOC₁₋₄alkyleneN(CH₃)₂, CO₂C₁₋₆alkyl, CO₂C₁₋₄alkyleneOC₁₋₆alkyl, NHC₁₋₄ alkyl, NC₁₋₄alkylC₁₋₄alkyl, NHC₁₋₂alkyleneC₃₋₁₀cycloalkyl, NCH₃Cl₁₋₂alkyleneC₃₋₁₀cycloalkyl, NCH₃Cl₁₋₂alkyleneC₃₋₁₀heterocycloalkyl, NHC₁₋₂alkyleneC₃₋₁₀heterocycloalkyl, NR²⁴C₁₋₄alkyleneOH, NR²⁴C₁₋₄alkyleneOCH₃, NHSO₂C₁₋₄alkyl, NCH₃SO₂C₁₋₄alkyl, and SO₂C₁₋₆alkyl, and alkyl, alkylene, phenyl, heteroaryl, heterocycloalkyl and cycloalkyl groups of R²⁰ are optionally substituted with one to three of R²⁵, and each R²⁵ is independently selected from F, C, and C₁₋₄alkyl. In an embodiment, each R²⁰ is independently selected from F, C, ═O, CN, OH, CH₃, CH₂CH₃, CH(CH₃)₂, C(CH₃)₃, CF₂H, CF₃, CFH₂, CH₂CFH₂, CH₂CF₂H, CH₂CF₃, CH₂CH₂CF₃, CH₂CH₂CF₂H, CH₂CH₂CH₂CF₂H, CH₂CH₂CH₂CF₃, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OC₁₋₄alkyl, C₁₋₄alkyleneOH, C₁₋₄alkyleneOCH₃, C(O)C₁₋₄alkyl, C(O)C₃₋₆cycloalkyl, C(O)C₃₋₆heterocycloalkyl, C(O)C₁₁aryl, C(O) C₅₋₁₄heteroaryl, C(O)C₁₋₂alkyleneC₃₋₆cycloalkyl, C(O)C₁₋₂alkyleneC₃₋₁₀heterocycloalkyl, C(O)C₁₋₄alkyleneC₆₋₁₁aryl, C(O)C₁₋₂alkyleneC₅₋₁₄heteroaryl, C(O)C₁₋₂alkyleneOCH₃, C(O)C₁₋₄alkyleneOH, C(O)C₁₋₄alkyleneNH₂, C(O)C₁₋₄alkyleneN(CH₃)₂, C(O)C₁₋₄alkyleneOC-4alkyleneN(CH₃)₂, CO₂C₁₋₆alkyl, CO₂C₁₋₄alkyleneOC₁₋₆alkyl, NHC₁₋₄alkyl, NC₁₋₄alkylC₁₋₄alkyl, NHC₁₋₂alkyleneC₃₋₁₀cycloalkyl, NCH₃Cl₁₋₂alkyleneC₃₋₁₀cycloalkyl, NHC₁₋₂alkylenephenyl, NHC₁₋₂alkyleneC₅₋₁₄heteroaryl, NHC₁₋₂alkylenephenyl, NHC₁₋₂alkyleneC₅₋₁₄heteroaryl, N(CH₃)C₁₋₂alkylenephenyl, N(CH₃)C₁₋₂alkyleneC₅₋₁₄heteroaryl, NCH₃Cl₁₋₂alkyleneC₃₋₁₀heterocycloalkyl, NHC₁₋₂alkyleneC₃₋₁₀heterocycloalkyl, NR²⁴C₁₋₄alkyleneOH, NR²⁴C₁₋₄alkyleneOCH₃, NHSO₂C₁₋₄alkyl, NCH₃SO₂C₁₋₄alkyl, and SO₂C₁₋₆alkyl, and alkyl, alkylene, heterocycloalkyl and cycloalkyl groups of R²⁰ are optionally substituted with one to three of R²⁵, and each R²⁵ is independently selected from F, C, and C₁₋₄alkyl. In an embodiment, each R²⁰ is independently selected from F, C, ═O, CN, OH, CH₃, CH₂CH₃, CH(CH₃)₂, C(CH₃)₃, CF₂H, CF₃, CFH₂, CH₂CFH₂, CH₂CF₂H, CH₂CF₃, CH₂CH₂CF₃, CH₂CH₂CH₂CF₃, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OC₁₋₄alkyl, C₁₋₄alkyleneOH, C₁₋₄alkyleneOCH₃, C(O)C₁₋₄alkyl, C(O)C₃₋₆cycloalkyl, C(O)C₃₋₆heterocycloalkyl, C(O)C₁₋₂alkyleneC₃₋₆cycloalkyl, C(O)C₁₋₂alkyleneC₃₋₁₀heterocycloalkyl, C(O)C₁₋₄alkyleneOCH₃, C(O)C₁₋₄alkyleneOH, C(O)C₁₋₄alkyleneNH₂, C(O)C₁₋₄alkyleneN(CH₃)₂, C(O)C₁₋₄alkyleneOC₁₋₄alkyleneN(CH₃)₂, CO₂C₁₋₆alkyl, CO₂C₁₋₄alkyleneOC₁₋₆alkyl, NHC₁₋₄alkyl, NC₁₋₄alkylC₁₋₄alkyl, NHC₁₋₂alkyleneC₃₋₁₀cycloalkyl, NCH₃C₁₋₂alkyleneC₃₋₁₀cycloalkyl, NCH₃C₁₋₂alkyleneC₃₋₁₀heterocycloalkyl, NHC₁₋₂alkyleneC₃₋₁₀heterocycloalkyl, NR²⁴C₁₋₄alkyleneOH, NR²⁴C₁₋₄alkyleneOCH₃, NHSO₂C₁₋₄alkyl, NCH₃SO₂C₁₋₄alkyl, and SO₂C₁₋₆alkyl, and alkyl, alkylene, and cycloalkyl groups of R²⁰ are optionally substituted with one to three of R²⁵, and each R²⁵ is independently selected from F, Cl, and C₁₋₄alkyl. In an embodiment, each R²⁰ is independently selected from CH₂CH₂CF₂H and CH₂CH₂CH₂CF₂H.

In an embodiment, each R²⁰ is independently selected from C₃₋₆cycloalkyl, C₃₋₆heteroycloalkyl, C₁₋₄alkyleneC₃₋₆cycloalkyl, and C₁₋₄alkyleneC₃₋₆heterocycloalkyl. In an embodiment, each R²⁰ is independently selected from C₃₋₅cycloalkyl, C₃₋₆heteroycloalkyl, C₁₋₄alkyleneC₃₋₅cycloalkyl, and C₁₋₄alkyleneC₃₋₅heterocycloalkyl. In an embodiments, all cycloalkyl groups of R²² are optionally substituted with one to three of R²⁵

In an embodiment, one R²⁰ is selected from cyclopropyl, cyclobutyl and cyclopentyl each of which are optionally substituted with one to three of R²⁵. In an embodiment, one R²⁰ is selected from cyclopropyl and cyclobutyl each of which are optionally substituted with one to three of R²⁵. In an embodiment, one R²⁰ is selected from cyclopropyl, cyclobutyl and cyclopentyl. In an embodiment, one R²⁰ is selected from cyclopropyl, cyclobutyl and cyclopentyl.

In an embodiment, one R²⁰ is selected from C₁₋₄alkylenecyclopropyl, C₁₋₄ alkylenecyclobutyl and C₁₋₄alkylenecyclopentyl and all cycloalkyl groups of R²⁰ are optionally substituted with one to three of R²⁵. In an embodiment, one R²⁰ is selected from C₁₋₄alkylenecyclopropyl, C₁₋₄alkylenecyclobutyl and C₁₋₄alkylenecyclopentyl. In an embodiment, one R²⁰ is selected from C₁₋₄alkylenecyclopropyl, C₁₋₄alkylenecyclobutyl and C₁₋₄alkylenecyclopentyl and all cycloalkyl groups of R²⁰ are optionally substituted with one to three of R²⁵. In an embodiment, one R²⁰ is selected from C₁₋₄alkylenecyclopropyl, C₁₋₄alkylenecyclobutyl and C₁₋₄alkylenecyclopentyl. In an embodiment, one R²⁰ is C₁₋₃alkylenecyclopropyl optionally substituted with one to three of R²⁵. In an embodiment, one R²⁰ is C₁₋₃alkylenecyclopropyl. In an embodiment, one R²⁰ is C₁₋₃alkylenecyclopropyl selected from

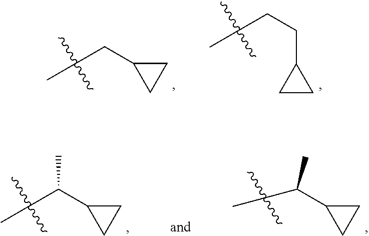

each of which is optionally substituted with one to three of R²⁵.

In an embodiment, one of R²⁰ is selected from phenyl, C₆₋₁₁heteroaryl, C₁₋₄alkyleneC₆₋₁₁aryl and C₁₋₄alkyleneC₅₋₁₄heteroaryl, and aryl and heteroaryl groups of R²⁰ are optionally substituted with one to three of R²⁵. In an embodiment, one of R²⁰ is selected from phenyl, C₅₋₇heteroaryl, C₁₋₄alkylenephenyl and C₁₋₄alkyleneC₅₋₇heteroaryl, and all aryl and heteroaryl groups of R²⁰ are optionally substituted with one to three of R²⁵. In an embodiment, one of R²⁰ is selected from phenyl and C₁₋₂alkylenephenyl optionally substituted with one to three of R²⁵. In an embodiment, one of R²⁰ is selected from C₅₋₇heteroaryl and C₁₋₆alkyleneC₅₋₇heteroaryl, and all heteroaryl groups of R²⁰ are optionally substituted with one to three of R²⁵. In an embodiment, the heteroaryl in C₅₋₇heteroaryl and C₁₋₆alkyleneC₅₋₇heteroaryl, of R²⁵ are selected from pyrrolyl, furanyl, thiophenyl, imidazolyl, pyrazolyl, oxathiolyl, isoxathiolyl, oxaxolyl, isoxazolyl, thiazoyl, isothiazolyl, triazolyl and tetrazolyl. In an embodiment, the heteroaryl in C₅₋₇heteroaryl and C₁₋₆alkyleneC₅₋₇heteroaryl, of R²⁵ is triazolyl. In an embodiment, the triazole is 1,2,3 trizole or 1, 2, 4-triazole.

In an embodiment, one R²⁰ is selected from CH₃, CH₂CH₃, CF₂H, CF₃, CFH₂, CH₂CF₂H, CH₂CH₂CF₂H, CH₂CH₂CH₂CF₂H and CH₂CF₃. In an embodiment, one R²⁰ is selected from CH₃, CH₂CH₃, CF₂H, CF₃, CFH₂, CH₂CF₂H and CH₂CF₃. In an embodiment, one R²⁰ is selected from CH₂CH₃ and CH(CH₃)₂. In an embodiment, one R²⁰ is selected from CH₂CH₃ and CH(CH₃)₂which are optionally fluoro-substituted. In an embodiment, each R²⁰ is independently selected from CH₃, CH₂CH₃, CF₂H, CF₃, CFH₂, CH₂CF₂H and CH₂CF₃. In an embodiment, each R²⁰ is independently selected from CH₂CH₃ and CH(CH₃)₂. In an embodiment, each R²⁰ is independently selected from CH₂CH₃ and CH(CH₃)₂ which are optionally fluoro-substituted. In an embodiment, one R²⁰ is selected CF₂H, CH₂CF₂H, CH₂CH₂CF₂H, and CH₂CH₂CH₂CF₂H.

In an embodiment, one of R²⁰ is selected from C₃₋₆heteroycloalkyl and C₁₋₄alkyleneC₃₋₆heterocycloalkyl. In an embodiment, the C₃₋₆heteroycloalkyl in the C₃₋₆heteroycloalkyl and C₁₋₄alkyleneC₃₋₆heterocycloalkyl of R²⁰ is selected from oxetanyl, tetrahydrofuranyl and tetrahydropyranyl.

In an embodiment, one R²⁰ is CO₂C₁₋₆alkyl. In an embodiment, one R²⁰ is CO₂C₁₋₆alkyl. In an embodiment, one R²⁰ is selected from CO₂CCH₃, CO₂CH₂CH₃, CO₂CF₂H, CO₂CF₃, CO₂CFH₂, CO₂CH₂CF₂H, CO₂CH₂CF₃, CO₂CH₂CH₂F₂H, CO₂CH₂CH₂CH₂F₂H, CO₂CH(CH₃)₂, and CO₂CH₂CH(CH₃)₂. an embodiment, one R²⁰ is CO₂CCH₃.

In an embodiment, one R²⁰ is COC₁₋₆alkyl. In an embodiment, one R²⁰ is COC₁₋₆alkyl. In an embodiment, one R²⁰ is selected from COCCH₃, COCH₂CH₃, COCF₂H, COCF₃, COCFH₂, COCH₂CF₂H, COCH₂CF₃, COCH₂CH₂F₂H, COCH₂CH₂CH₂F₂H, COCH(CH₃)₂, and COCH₂CH(CH₃)₂. an embodiment, one R²⁰ is COCH₃.

In an embodiment, each R²⁰ is independently selected from OH, F, Cl, CF₃, CF₂H, CH₃, CH₂CH₃, CH₂CF₂H, CH₂CH₂CF₂H, CH₂CH₂CH₂F₂H

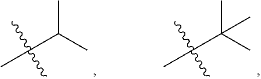

OC₁₋₄alkyl, SO₂C₁₋₄alkyl,

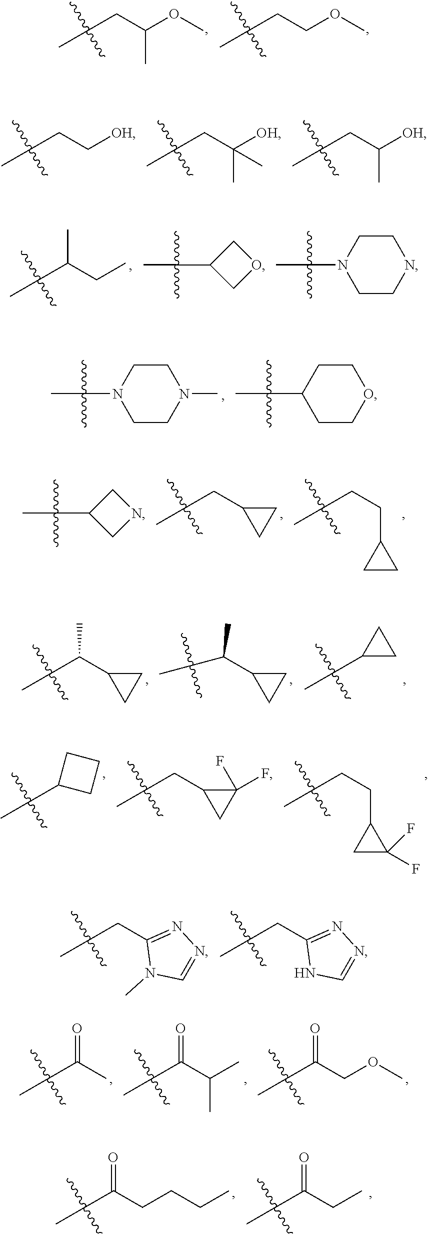

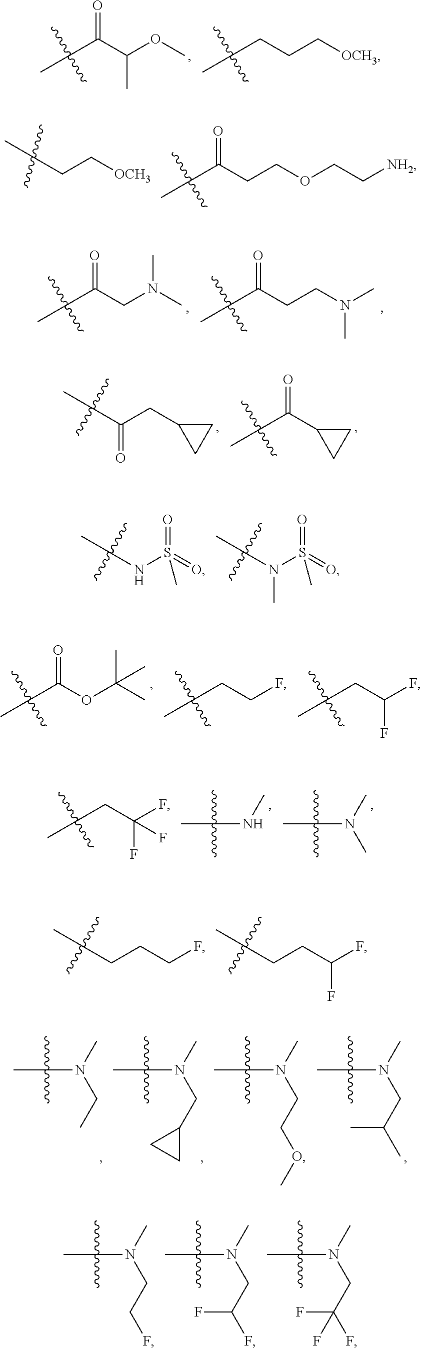

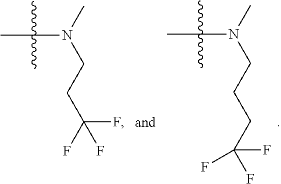

In an embodiment, each R²⁰ is independently selected from OH, F, Cl, CF₃, CH₃, CH₂CH₃,

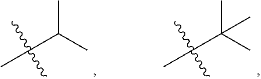

OC₁₋₄alkyl, SO₂C₁₋₄alkyl,

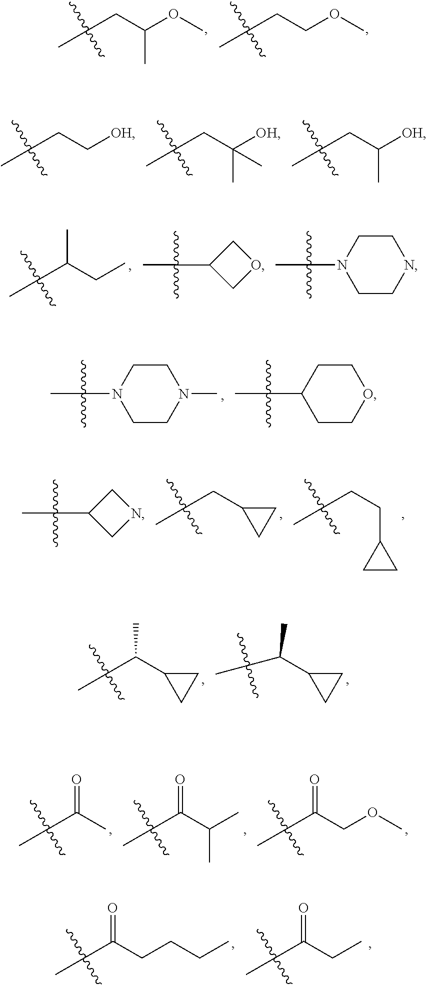

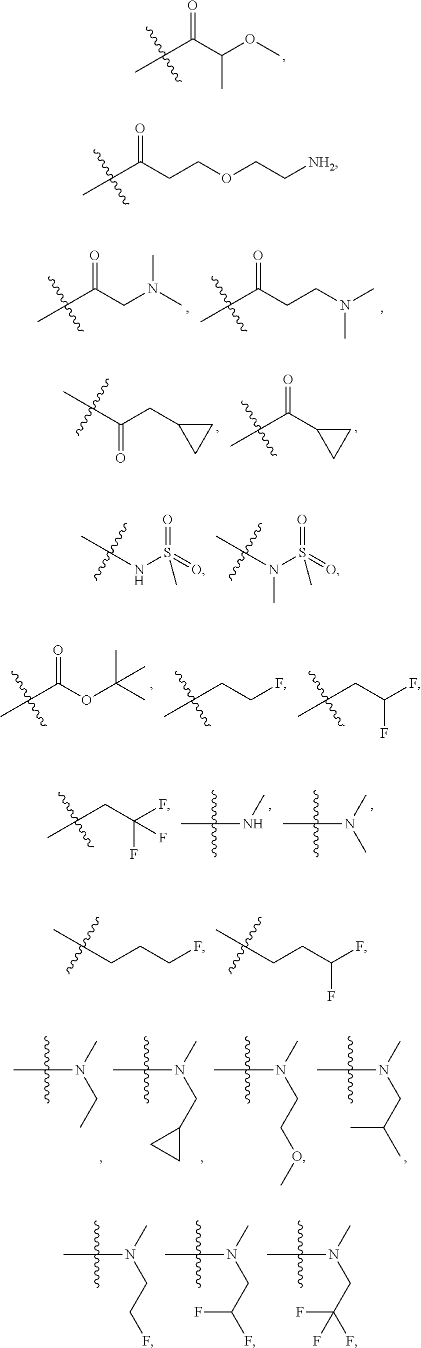

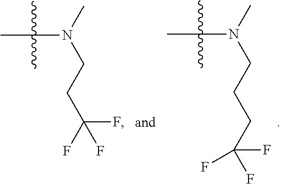

In an embodiment, R²¹ is selected from H, C₁₋₄alkyl, C₁₋₄alkyleneOC₁₋₆alkyl, C₃₋₁₁cycloalkyl, C₃₋₁₀heterocycloalkyl, C₆₋₁₁aryl, C₅₋₁₄heteroaryl, C₂₋₄alkenyl, C₂₋₄alkynyl, C₁₋₄alkyleneC₃₋₁₀cycloalkyl, C₁₋₄alkyleneC₃₋₁₀heterocycloalkyl, C₁₋₄alkyleneC₆₋₁₁aryl and C₁₋₄alkyleneC₅₋₁₄heteroaryl. In an embodiment, R²¹ is selected from H and C₁₋₄alkyl. In an embodiment, R²¹ is selected from C₁₋₄alkyleneOC₁₋₆alkyl, C₁₋₄alkyleneC₃₋₁₀cycloalkyl, and C₁₋₄alkyleneC₃₋₁₀heterocycloalkyl. In an embodiment, R²¹ is selected from H, C₁₋₄alkyl, C₁₋₄alkyleneOC₁₋₆alkyl, C₃₋₁₁cycloalkyl, C₃₋₁₀heterocycloalkyl, C₂₋₄alkenyl, C₂₋₄alkynyl, C₁₋₄alkyleneC₃₋₁₀cycloalkyl, and C₁₋₄alkyleneC₃₋₁₀heterocycloalkyl. In an embodiment, R²¹ is selected from H and C₁₋₄alkyl. In an embodiment, R²¹ is selected from C₁₋₄alkyleneOC₁₋₆alkyl, C₁₋₄alkyleneC₃₋₁₀cycloalkyl, and C₁₋₄alkyleneC₃₋₁₀heterocycloalkyl.

In an embodiment, R²² is selected from H and C₁₋₄alkyl.

In an embodiment, R²³ is selected from H, C₁₋₄alkyl, C₁₋₄alkyleneOC₁₋₄alkyl, C₃₋₁₀cycloalkyl, C₃₋₁₀heterocycloalkyl, C₆₋₁₁aryl, C₅₋₁₄heteroaryl, C₁₋₄alkyleneC₃₋₁₀cycloalkyl, C₁₋₄alkyleneC₃₋₁₀heterocycloalkyl, C₁₋₄alkyleneC₆₋₁₁aryl and C₁₋₄alkyleneC₅₋₁₄heteroaryl. In an embodiment, R²³ is selected from H, C₁₋₄alkyl, C₁₋₄alkyleneOC₁₋₄alkyl, C₃₋₁₀cycloalkyl, C₃₋₁₀heterocycloalkyl, C₁₋₄alkyleneC₃₋₁₀cycloalkyl, and C₁₋₄alkyleneC₃₋₁₀heterocycloalkyl.

In an embodiment, R²² and R²³ are each independently selected from H and C₁₋₄alkyl. In an embodiment, R²² and R²³ are joined to form, together with the nitrogen atom therebetween, a 4- to 6-membered saturated or unsaturated ring, optionally containing one additional heteromoiety selected from N, NR²⁶, O, S, S(O), and SO₂ and optionally substituted with one to three of halo and C₁₋₆alkyl.

In an embodiment, R²⁴ and R²⁶ are independently selected from H and C. ₄alkyl.

In an embodiment, each R²⁵ is independently selected from F, Cl, CN, C. ₄alkyl and NR^{25a}R^{25b}. In an embodiment, each R²⁵ is independently selected from F, C₁₋₄alkyl and NR^{25a}R^{25b}. In an embodiment, at least one of R²⁵ is F. In an embodiment, one of R²⁵ is NR^{25a}R^{25b}.

In an embodiment, R^{25a} and R^{25b} are independently selected from H and C. ₄alkyl. In an embodiment, R^{25a} and R^{25b} are independently selected from H, CH₃, CH₂CH₃, CF₂H, CF₃, CFH₂, CH₂CF₂H and CH₂CF₃. In an embodiment, R^{25a} and R^{25b} are independently selected from H, CF₃ and CH₃. In an embodiment, R^{25a} and R^{25b} are independently selected from H and CH₃.

In an embodiment, Cy² is pyrrolidinyl, piperidinyl, piperazinyl or morpholinyl, and each R²⁰ is independently CH₃, CH₂CH₃, CF₂H, CF₃, CFH₂, CH₂CF₂H and CH₂CF₃. In an embodiment, Cy² is pyrrolidinyl, piperidinyl, piperazinyl or morpholinyl, and each R²⁰ is independently CH₂CH₃ or CH(CH₃)₂.

In an embodiment, Cy² is pyrrolidinyl, piperidinyl, piperazinyl or morpholinyl, and one of R²⁰ is C₁₋₃alkylenecyclopropyl.

In an embodiment, the compound of Formula (I) is a compound of Formula (I-A) or (I-B) or a pharmaceutically acceptable salt, solvate, and/or prodrug thereof:

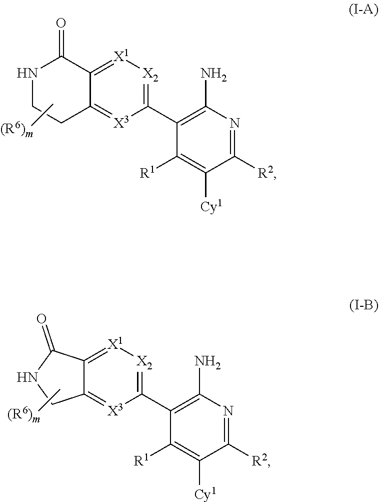

wherein R¹ R², R⁶, X¹, X², X³, and Cy¹ are as defined in Formula (I) above; and
m is an integer selected from 0 to 4.

In an embodiment, the compound of Formula (I) is a compound of Formula (I-C) or a pharmaceutically acceptable salt, solvate, and/or prodrug thereof:

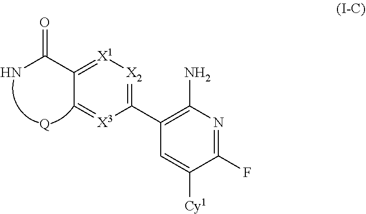

wherein Q, X¹, X², X³, and Cy¹ are as defined in Formula (I) above.

In an embodiment, the compound of Formula (I) is a compound of Formula (I-D), or (I-E), or a pharmaceutically acceptable salt, solvate, and/or prodrug thereof:

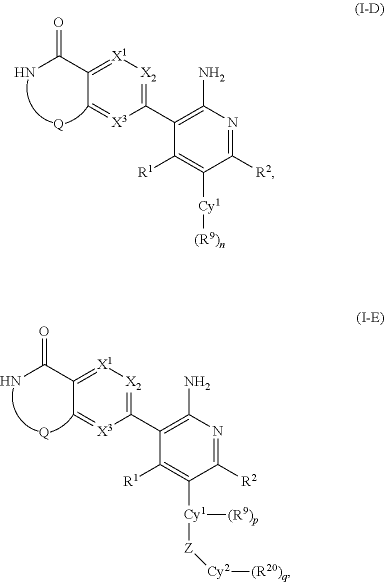

wherein Q, R¹, R², R⁹, R²⁰, X¹, X², X³, Z, Cy¹, and Cy² are as defined in Formula (I) above;
n is an integer selected from 0 to 5; and
p and q are integers independently selected from 0 to 4.

In an embodiment, the compound of Formula (I) is a compound of Formula (I-F) or (I-G), or a pharmaceutically acceptable salt, solvate, and/or prodrug thereof:

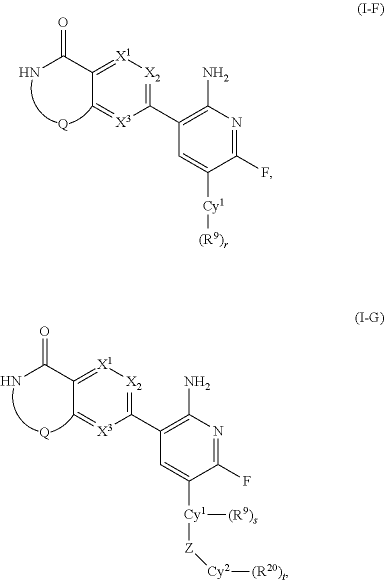

wherein Q, R⁹, R²⁰, X¹, X², X³, Z, Cy¹, and Cy² are as defined in Formula (I) above;
r is an integer selected from 0 to 5; and
s and t are integers independently selected from 0 to 4.

In an embodiment, the compound of Formula (I) is a compound of Formula (I-H), or a pharmaceutically acceptable salt, solvate, and/or prodrug thereof:

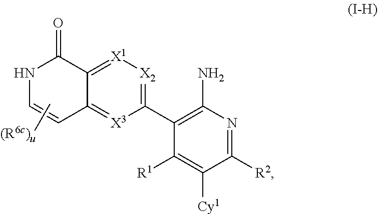

wherein R¹, R², R^{6c}, X¹, X², X³, and Cy¹ are as defined in Formula (I) above; and
u is an integer selected from 0 to 2.

In an embodiment, the compound of Formula (I) is a compound of Formula (I-J) or (I-K) or a pharmaceutically acceptable salt, solvate, and/or prodrug thereof:

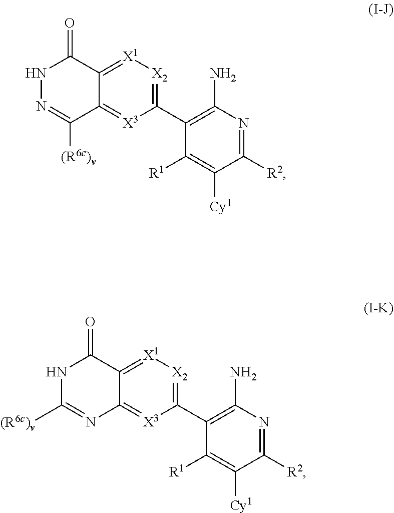

wherein R¹, R², R^{6c} , X¹, X², X³, and Cy¹ are as defined in Formula (I) above; and
v is an integer selected from 0 and 1.

In an embodiment, the compound of Formula (I) is a compound of Formula (I-L) or (I-M) or a pharmaceutically acceptable salt, solvate, and/or prodrug thereof:

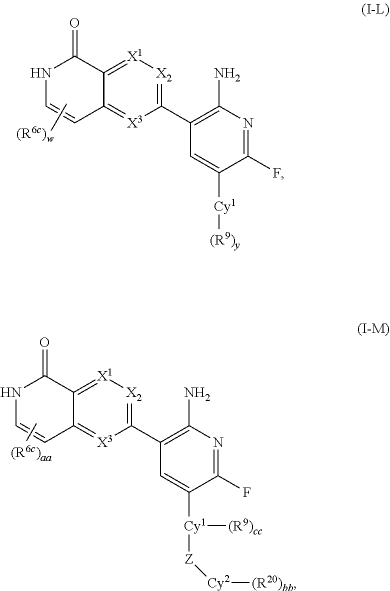

wherein R^{6c} , R⁹, X¹, X², X³, Z, Cy¹ and Cy² are as defined in Formula (I) above;
w and aa are integers independently selected from 0 to 2,
y and bb are integers independently selected from 0 to 4, and
cc is an integer selected from 0 to 5.

In an embodiment, R^{6c} in the formula of (I-L) or (I-M) is selected from F, Cl, OH, C₁₋₄alkyl, OC₁₋₄alkyl and NR^{7a}R^{8a}. In an embodiment, one or two R^{6c} are selected from F, Cl, CH₃, CF₂H, CF₃, OCH₃, OCF₃, OCF₂H and NR^{7a}R^{8a}

In an embodiment, the compound of Formula (I) is a compound of Formula (I-N) or (I-O) or a pharmaceutically acceptable salt, solvate, and/or prodrug thereof:

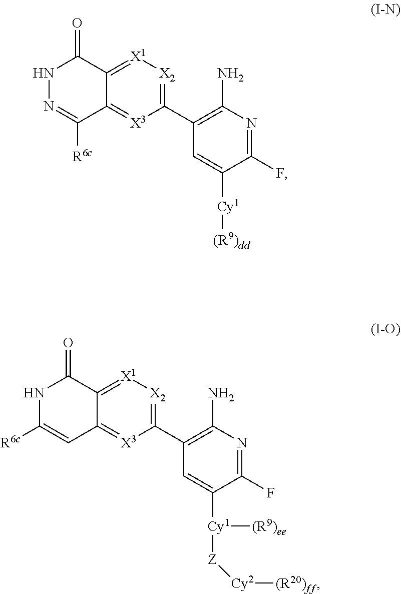

wherein R^{6c} , R⁹, X¹, X², X³, Z, Cy¹ and Cy² are as defined in Formula (I) above;
dd and ee are integers independently selected from 0 to 4, and
ff is an integer selected from 0 to 5.

In an embodiment, the compound of Formula (I) is a compound of Formula (I-P) or (I-Q), or a pharmaceutically acceptable salt, solvate, and/or prodrug thereof:

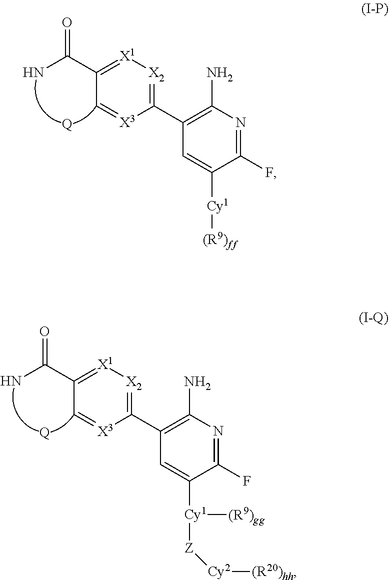

wherein Q, R²⁰, X¹, X², X³, Z, Cy¹, and Cy² are as defined in Formula (I) above;
R⁹ is selected from C₁₋₄alkyleneNR^{10a}R^{11a}, NR^{10a}R^{11a}, NR¹¹COR¹⁰, SO₂NR¹⁰R¹¹, NR¹¹C₁₋₄alkyleneNR¹⁰R¹¹, C₃₋₇heterocycloalkyl, and C₁₋₄alkyleneC₃₋₇heterocycloalkyl, the latter two groups being optionally substituted with one to four of R¹³,
R¹⁰, R¹⁰, R¹¹, R^{11a}, R¹³ are as defined in Formula (I) above;
ff is an integer selected from 1 to 5; and
gg is an integer selected from 1 to 4, and
hh is an integer selected from 0 to 5.

In an embodiment, one R⁹ in the of Formula (I-P) or (I-Q) is selected from C₁₋₄alkyleneNR^{10a}R^{11a}, NR^{10a}R^{11a}, NR¹¹COR¹⁰, NR¹¹C₁₋₄alkyleneNR¹⁰R¹¹, C₃₋₇heterocycloalkyl, and C₁₋₄alkyleneC₃₋₇heterocycloalkyl, the latter two groups being optionally substituted with one to four of R¹³ as defined above for Formula (I). In an embodiment, one or two R⁹ in the of Formula (I-P) or (I-Q) is are selected from C₁₋₄alkyleneNR^{10a}R^{11a}, NR^{10a}R^{11a}, C₃₋₇heterocycloalkyl, and C₁₋₄alkyleneC₃₋₇heterocycloalkyl, the latter two groups being optionally substituted with one to four of R¹³ as described above for Formula (I). In an embodiment, one R⁹ in the of Formula (I-P) or (I-Q) is C₁₋₂alkyleneNR^{10a}R^{11a} as described above for Formula (I). In an embodiment ff and gg are 1 or 2, suitably 1.

In an embodiment, the compound of Formula (I) is a compound of Formula (I-R) or (I-S) or a pharmaceutically acceptable salt, solvate, and/or prodrug thereof:

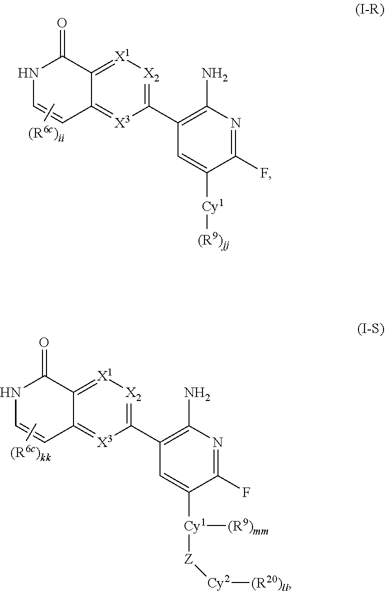

wherein R^{6c}, X¹, X², X³, Z, Cy¹ and Cy² are as defined in Formula (I) above;
R⁹ is selected from C₁₋₄alkyleneNR^{10a}R^{11a}, NR^{10a}R^{11a}, NR¹¹COR¹⁰, SO₂NR¹⁰R¹¹, NR¹¹C₁₋₄alkyleneNR¹⁰R¹¹, C₃₋₇heterocycloalkyl, and C₁₋₄alkyleneC₃₋₇heterocycloalkyl, the latter two groups being optionally substituted with one to four of R¹³
R¹⁰, R¹⁰, R¹¹, R^{11a}, R¹³ are as defined in Formula (I) above;
ii and kk are integers independently selected from 0 to 2,
jj and mm are integers independently selected from 1 to 4, and
II is an integer selected from 0 to 5.

In an embodiment, ii and kk are both 1, and R^{6c}in the formula of (I-R) or (I-S) is selected from F and Cl. In an embodiment, ii and kk are both 1, R^{6c} in the formula of (I-R) or (I-S is F.

In an embodiment, jj is 1 or 2, suitably 1. In an embodiment, II is 1 or 2, suitably 1. In an embodiment, one R⁹ in the of Formula (I-R) or (I-S) s selected from C₁₋₄alkyleneNR^{10a}R^{11a}, NR^{10a}R^{11a}, NR¹¹COR¹⁰, NR¹¹C₁₋₄alkyleneNR¹⁰R¹¹, C₃₋₇heterocycloalkyl, and C₁₋₄alkyleneC₃₋₇heterocycloalkyl, the latter two groups being optionally substituted with one to four of R¹³ as defined above for Formula (I). In an embodiment, one or two R⁹ in the of Formula (I-R) or (I-S) are selected from C₁₋₄alkyleneNR^{10a}R^{11a}, NR^{10a}R^{11a}, C₃₋₇heterocycloalkyl, and C₁₋₄alkyleneC₃₋₇heterocycloalkyl, the latter two groups being optionally substituted with one to four of R¹³ as defined above for Formula (I). In an embodiment, one R⁹ in the of Formula (I-R) or (I-S) is C₁₋₂alkyleneNR^{10a}R^{11a} as defined above for Formula (I).

In an embodiment, the compound of Formula (I) is a compound of Formula (I-T) or (I-U) or a pharmaceutically acceptable salt, solvate, and/or prodrug thereof:

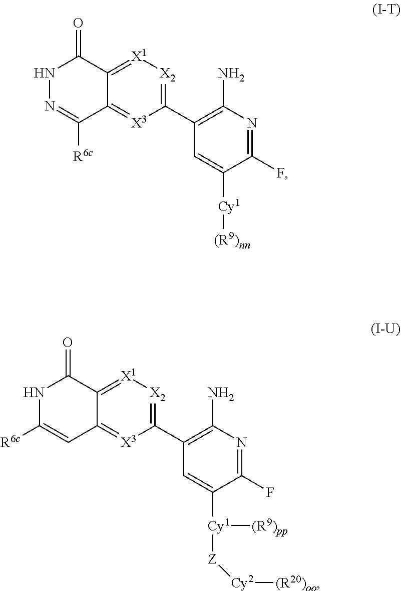

wherein R^{6c} , X¹, X², X³, Z, Cy¹ and Cy² are as defined in Formula (I) above;
R⁹ is selected from C₁₋₄alkyleneNR^{10a}R^{11a}, NR^{10a}R^{11a}, NR¹¹COR¹⁰, SO₂NR¹⁰R¹¹, NR¹¹C₁₋₄alkyleneNR¹⁰R¹¹, C₃₋₇heterocycloalkyl, and C₁₋₄alkyleneC₃₋₇heterocycloalkyl, the latter two groups being optionally substituted with one to four of R¹³
R¹⁰, R^{10a}, R¹¹, R^{11a}, R¹³ are as defined in Formula (I) above;
nn and pp are integers independently selected from 1 to 4, and
oo is an integer selected from 0 to 5.

In an embodiment, nn is 1 or 2, suitably 1. In an embodiment, oo is 1 or 2, suitably 1. In an embodiment, one R⁹ in the of Formula (I-T) or (I-U) is selected from C₁₋₄alkyleneNR^{10a}R^{11a} NR^{10a}R^{11a}, NR¹¹R, NR¹¹C₁₋₄alkyleneNR¹⁰R¹¹, C₃₋₇heterocycloalkyl, and C₁₋₄alkyleneC₃₋₇heterocycloalkyl, the latter two groups being optionally substituted with one to four of R¹³ as defined above for Formula (I). In an embodiment, one or two R⁹ in the of Formula (I-T) or (I-U) are selected from C₁₋₄alkyleneNR^{10a}R^{11a}, NR^{10a}R^{1a}, C₃₋₇heterocycloalkyl, and C₁₋₄alkyleneC₃₋₇heterocycloalkyl, the latter two groups being optionally substituted with one to four of R¹³ as defined above for Formula (I). In an embodiment, one R⁹ in the of Formula (I-T) or (I-U) is C₁₋₂alkyleneNR^{10a}R^{11a} as defined above for Formula (I).

In an embodiment, the compound of Formula (I) is selected from the compounds listed in Table 1 below:

TABLE 1

| Compound | | |
|---|---|---|
| ID # | IUPAC Name | Structures |
| I-1 | 6-(2-amino-4-fluoro-5-(4-(4- methylpiperazin-1- yl)phenyl)pyridin-3-yl)-3,4- dihydroisoqinolin-1(2H)- one | 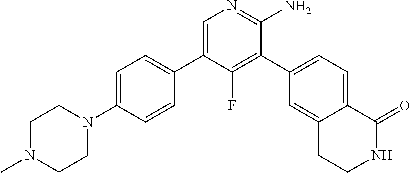 [SMILES: CN1CCN(c2ccc(-c3cnc(N)c(-c4ccc5c(c4)CCNC5=O)c3F)cc2)CC1] |
| I-2 | 6-(2-amino-4-fluoro-5-(4- morpholinophenyl)pyridin-3- yl)-3,4-dihydroisoquinolin- 1(2H)-one | 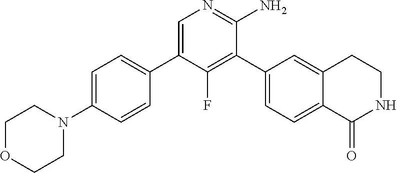 [SMILES: Nc1ncc(-c2ccc(N3CCOCC3)cc2)c(F)c1-c1ccc2c(c1)CCNC2=O] |
| I-3 | 6-(2-amino-4-fluoro-5-(4- (isopropylsulfonyl)phenyl)pyri- din-3-yl)-3,4- dihydroisoquinolin-1(2H)- one | 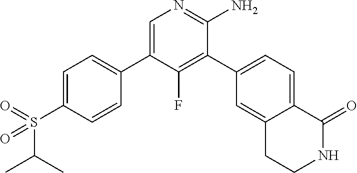 [SMILES: CC(C)S(=O)(=O)c1ccc(-c2cnc(N)c(-c3ccc4c(c3)CCNC4=O)c2F)cc1] |
| I-4 | (R)-6-(2-amino-4-fluoro-5-(4- (2- isopropylmorpholino)phenyl) pyridin-3-yl)-3,4- dihydroisoquinolin-1(2H)- one | 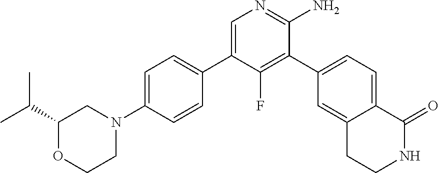 [SMILES: CC(C)[C@@H]1CN(c2ccc(-c3cnc(N)c(-c4ccc5c(c4)CCNC5=O)c3F)cc2)CCO1] |
| I-5 | (S)-6-(2-amino-4-fluoro-5-(4- (2- isopropylmorpholino)phenyl) pyridin-3-yl)-3,4- dihydroisoquinolin-1(2H)- one | 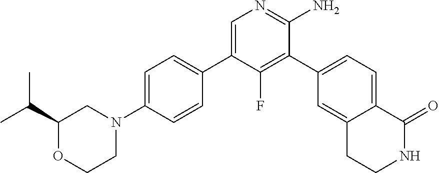 [SMILES: CC(C)[C@H]1CN(c2ccc(-c3cnc(N)c(-c4ccc5c(c4)CCNC5=O)c3F)cc2)CCO1] |
| I-6 | (R)-6-(2-amino-4-fluoro-5-(2- fluoro-4-(2- isopropylmorpholino)phenyl) pyridin-3-yl)-3,4- dihydroisoquinolin-1(2H)- one | 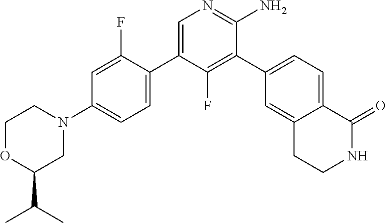 [SMILES: CC(C)[C@@H]1CN(c2ccc(-c3cnc(N)c(-c4ccc5c(c4)CCNC5=O)c3F)c(F)c2)CCO1] |
| I-7 | (S)-6-(2-amino-4-fluoro-5- (2-fluoro-4-(2- isopropylmorpholino)phenyl) pyridin-3-yl)-3,,4- dihydroisoquinolin-1(2H)- one | 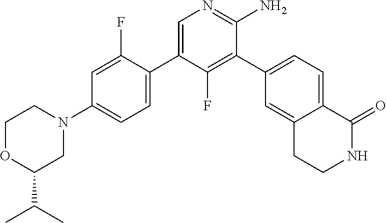 [SMILES: CC(C)[C@H]1CN(c2ccc(-c3cnc(N)c(-c4ccc5c(c4)CCNC5=O)c3F)c(F)c2)CCO1] |
| I-8 | 6-(2-amino-6-fluoro-5-(4-(4- methylpiperazin-1- yl)phenyl)pyridin-3-yl)-3,4- dihydroisoquinolin-1(2H)- one | 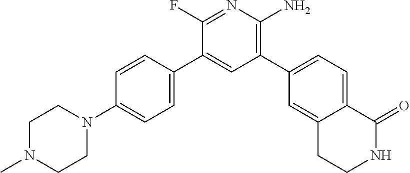 [SMILES: CN1CCN(c2ccc(-c3cc(-c4ccc5c(c4)CCNC5=O)c(N)nc3F)cc2)CC1] |
| I-9 | (R)-6-(2-amino-6-fluoro-5-(4- (2- isopropylmorpholino)phenyl) pyridin-3-yl)-3,4- dihydroisoquinolin-1(2H)- one | 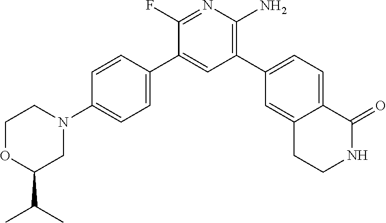 [SMILES: CC(C)[C@@H]1CN(c2ccc(-c3cc(-c4ccc5c(c4)CCNC5=O)c(N)nc3F)cc2)CCO1] |
| I-10 | (S)-6-(2-amino-6-fluoro-5-(4- (2- isopropylmorpholino)phenyl) pyridin-3-yl)-3,4- dihydroisoquinolin-1(2H)- one | 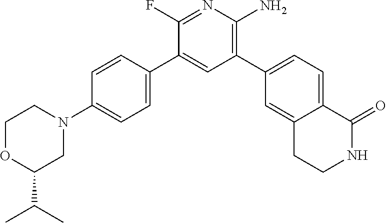 [SMILES: CC(C)[C@H]1CN(c2ccc(-c3cc(-c4ccc5c(c4)CCNC5=O)c(N)nc3F)cc2)CCO1] |
| I-11 | (R)-6-(2-amino-6-fluoro-5-(2- fluoro-4-(2- isopropylmorpholino)phenyl) pyridin-3-yl)-3,4- dihydroisoquinolin-1(2H)- one | 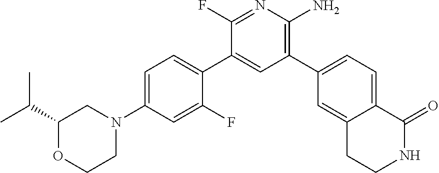 [SMILES: CC(C)[C@@H]1CN(c2ccc(-c3cc(-c4ccc5c(c4)CCNC5=O)c(N)nc3F)c(F)c2)CCO1] |
| I-12 | (S)-6-(2-amino-6-fluoro-5- (2-fluoro-4-(2- isopropylmorpholino)phenyl) pyridin-3-yl)-3,4- dihydroisoquinolin-1(2H)- one | 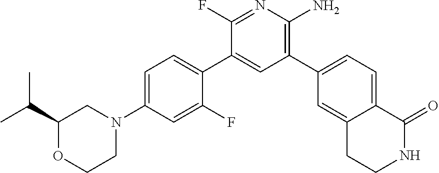 [SMILES: CC(C)[C@H]1CN(c2ccc(-c3cc(-c4ccc5c(c4)CCNC5=O)c(N)nc3F)c(F)c2)CCO1] |
| I-13 | 6-(2-amino-6-fluoro-5-(4- ((1R,5S)-8-methyl-3,8- diazabicyclo[3.2.1]octan-3- yl)phenyl)pyridin-3-yl)-3,4- dihydroisoquinolin-1(2H)- one | 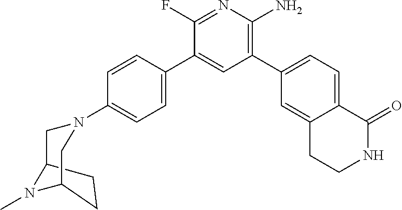 [SMILES: CN1C2CCC1CN(c1ccc(-c3cc(-c4ccc5c(c4)CCNC5=O)c(N)nc3F)cc1)C2] |
| I-14 | 6-(2-amino-6-fluoro-5-(4- ((1R,5S)-3-methyl-3- azabicyclo[3.1.0]hexan-1- yl)phenyl)pyridin-3-yl)-3,4- dihydroisoquinolin-1(2H)- one | 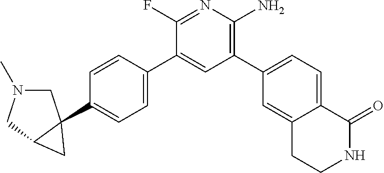 [SMILES: CN1C[C@H]2C[C@@]2(c2ccc(-c3cc(-c4ccc5c(c4)CCNC5=O)c(N)nc3F)cc2)C1] |
| I-15 | 6-(2-amino-6-fluoro-5-(4- (isopropylsulfonyl)phenyl)pyri- din-3-yl)-3,4- dihydroisoquinolin-1(2H)- one | 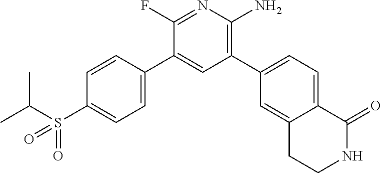 [SMILES: CC(C)S(=O)(=O)c1ccc(-c2cc(-c3ccc4c(c3)CCNC4=O)c(N)nc2F)cc1] |
| I-16 | 6-(2-amino-6-fluoro-5-(4- morpholinophenyl)pyridin-3- yl)-3,4-dihydroisoquinolin- 1(2H)-one | 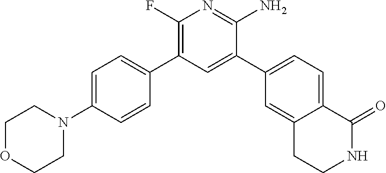 [SMILES: Nc1nc(F)c(-c2ccc(N3CCOCC3)cc2)cc1-c1ccc2c(c1)CCNC2=O] |
| I-17 | 6-(2-amino-6-fluoro-5-(4- (piperazin-1- yl)phenyl)pyridin-3-yl)-3,4- dihydroisoquinolin-1(2H)- one | 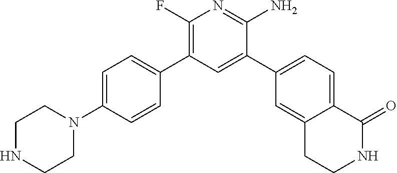 [SMILES: Nc1nc(F)c(-c2ccc(N3CCNCC3)cc2)cc1-c1ccc2c(c1)CCNC2=O] |
| I-18 | 6-(2-amino-6-fluoro-5-(4-(5- methyl-2,5- diazabicyclo[2.2.1]heptan-2- yl)phenyl)pyridin-3-yl)-3,4- dihydroisoquinolin-1(2H)- one | 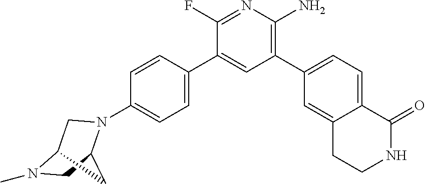 [SMILES: CN1C[C@@H]2C[C@H]1CN2c1ccc(-c2cc(-c3ccc4c(c3)CCNC4=O)c(N)nc2F)cc1] |
| I-19 | 6-(2-amino-6-fluoro-5-(4- morpholinophenyl)pyridin-3- yl)-7-fluoro-3,4- dihydroisoquinolin-1(2H)- one | 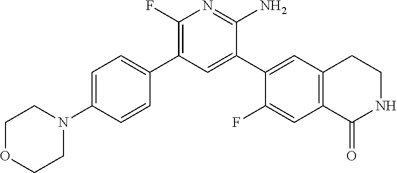 [SMILES: Nc1nc(F)c(-c2ccc(N3CCOCC3)cc2)cc1-c1cc2c(cc1F)C(=O)NCC2] |
| I-20 | 6-(2-amino-6-fluoro-5-(4-(4- (2-fluoroethyl)piperazin-1- yl)phenyl)pyridin-3-yl)-3,4- dihydroisoquinolin-1(2H)- one | 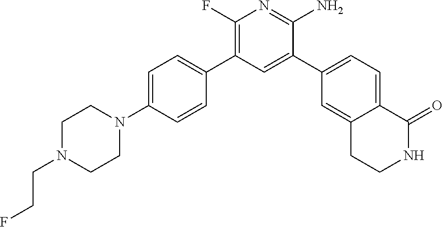 [SMILES: Nc1nc(F)c(-c2ccc(N3CCN(CCF)CC3)cc2)cc1-c1ccc2c(c1)CCNC2=O] |
| I-21 | 6-(2-amino-5-(4-(4-(2,2- difluoroethyl)piperazin-1- yl)phenyl)-6-fluoropyridin-3- yl)-3,4-dihydroisoquinolin- 1(2H)-one | 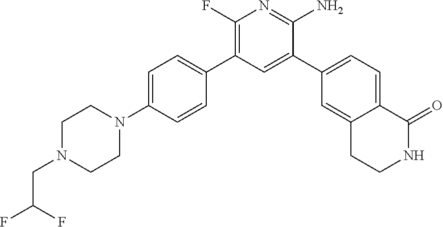 [SMILES: Nc1nc(F)c(-c2ccc(N3CCN(CC(F)F)CC3)cc2)cc1-c1ccc2c(c1)CCNC2=O] |
| I-22 | 6-(2-amino-6-fluoro-5-(4- (piperidin-4- yloxy)phenyl)pyridin-3-yl)- 3,4-dihydroisoquinolin- 1(2H)-one | 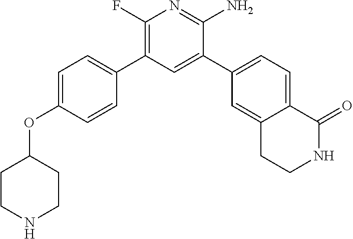 [SMILES: Nc1nc(F)c(-c2ccc(OC3CCNCC3)cc2)cc1-c1ccc2c(c1)CCNC2=O] |
| I-23 | 6-(2-amino-6-fluoro-5-(4- (hexahydropyrazino[2,1- c][1,4]oxazin-8(1H)- yl)phenyl)pyridin-3-yl)-3,4- dihydroisoquinolin-1(2H)- one | 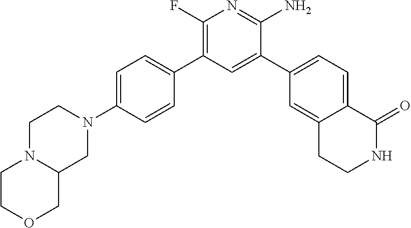 [SMILES: Nc1nc(F)c(-c2ccc(N3CCN4CCOCC4C3)cc2)cc1-c1ccc2c(c1)CCNC2=O] |
| I-24 | (S)-6-(2-amino-6-fluoro-5-(4- (4-(2- methoxypropyl)piperazin-1- yl)phenyl)pyridin-3-yl)-3,4- dihydroisoquinolin-1(2H)- one | 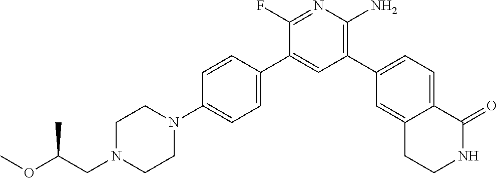 [SMILES: CO[C@@H](C)CN1CCN(c2ccc(-c3cc(-c4ccc5c(c4)CCNC5=O)c(N)nc3F)cc2)CC1] |
| I-25 | (R)-6-(2-amino-6-fluoro-5- (4-(4-(2- methoxypropyl)piperazin-1- yl)phenyl)pyridin-3-yl)-3,4- dihydroisoquinolin-1(2H)- one | 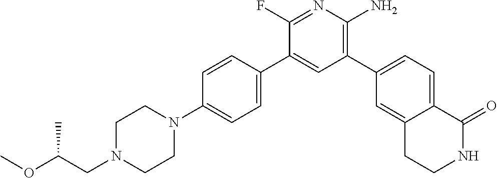 [SMILES: CO[C@H](C)CN1CCN(c2ccc(-c3cc(-c4ccc5c(c4)CCNC5=O)c(N)nc3F)cc2)CC1] |
| I-26 | 6-(6′-amino-2′-fluoro-5- morpholino-[2,3′-bipyridin]- 5′-yl)-3,4-dihydroisoquinolin- 1(2H)-one | 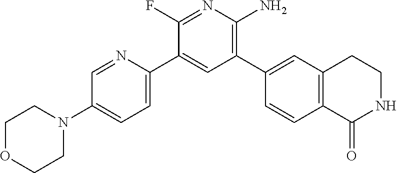 [SMILES: Nc1nc(F)c(-c2ccc(N3CCOCC3)cn2)cc1-c1ccc2c(c1)CCNC2=O] |
| I-27 | 6-(6′-amino-2′-fluoro-5-(4- methylpiperazin-1-yl)-[2,3′- bipyridin]-5′-yl)-3,4- dihydroisoquinolin-1(2H)- one acetate | 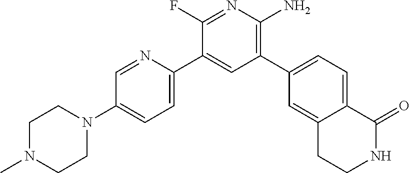 [SMILES: CN1CCN(c2ccc(-c3cc(-c4ccc5c(c4)CCNC5=O)c(N)nc3F)nc2)CC1] |
| I-28 | 6-(2-amino-6-fluoro-5-(4- (hydroxydimethylsilyl)phenyl) pyridin-3-yl)-3,4- dihydroisoquinolin-1(2H)- one | 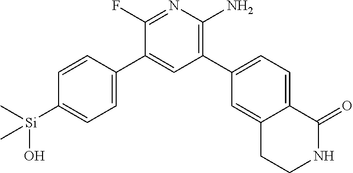 [SMILES: C[Si](C)(O)c1ccc(-c2cc(-c3ccc4c(c3)CCNC4=O)c(N)nc2F)cc1] |
| I-29 | 6-(2-amino-6-fluoro-5-(4-(4- (2-methoxyethyl)piperazin-1- yl)phenyl)pyridin-3-yl)-3,4- dihydroisoquinolin-1(2H)- one | 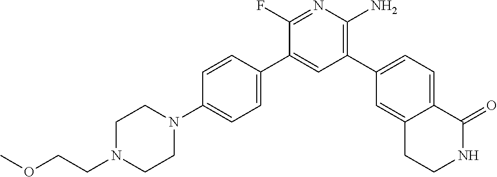 [SMILES: COCCN1CCN(c2ccc(-c3cc(-c4ccc5c(c4)CCNC5=O)c(N)nc3F)cc2)CC1] |
| I-30 | 6-(2-amino-6-fluoro-5-(4- (piperidin-4- yl)phenyl)pyridin-3-yl)-3,4- dihydroisoquinolin-1(2H)- one | 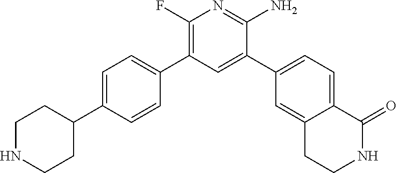 [SMILES: Nc1nc(F)c(-c2ccc(C3CCNCC3)cc2)cc1-c1ccc2c(c1)CCNC2=O] |
| I-31 | 6-(2-amino-6-fluoro-5-(4-(1- methyl-5,6-dihydro-1,2,4- triazin-4(1H)- yl)phenyl)pyridin-3-yl)-3,4- dihydroisoquinolin-1(2H)- one | 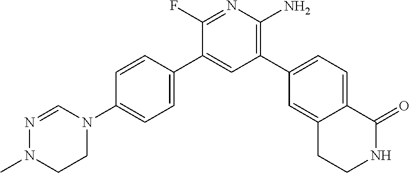 [SMILES: CN1CCN(c2ccc(-c3cc(-c4ccc5c(c4)CCNC5=O)c(N)nc3F)cc2)C=N1] |
| I-32 | 5-(2-amino-6-fluoro-5-(4-(4- methylpiperazin-1- yl)phenyl)pyridin-3- yl)isoindolin-1-one | 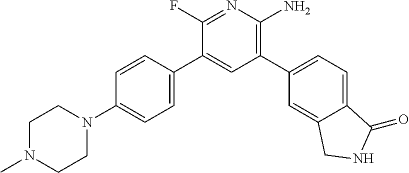 [SMILES: CN1CCN(c2ccc(-c3cc(-c4ccc5c(c4)CNC5=O)c(N)nc3F)cc2)CC1] |
| I-33 | 5-(2-amino-6-fluoro-5-(4-(4- methylpiperazin-1- yl)phenyl)pyridin-3-yl)-3,3- dimethylisoindolin-1-one | 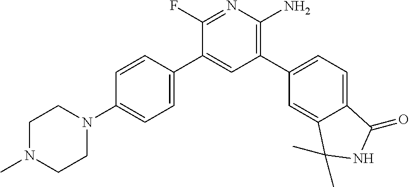 [SMILES: CN1CCN(c2ccc(-c3cc(-c4ccc5c(c4)C(C)(C)NC5=O)c(N)nc3F)cc2)CC1] |
| I-34 | 6-(5-(4-((1-acetylpiperidin-4- yl)oxy)phenyl)-2-amino-6- fluoropyridin-3-yl)-3,4- dihydroisoquinolin-1(2H)- one | 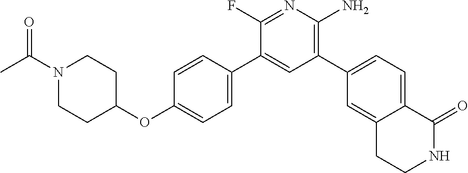 [SMILES: CC(=O)N1CCC(Oc2ccc(-c3cc(-c4ccc5c(c4)CCNC5=O)c(N)nc3F)cc2)CC1] |
| I-35 | 6-(2-amino-6-fluoro-5-(4- ((tetrahydro-2H-pyran-4- yl)oxy)phenyl)pyridin-3-yl)- 3,4-dihydroisoquinolin- 1(2H)-one | 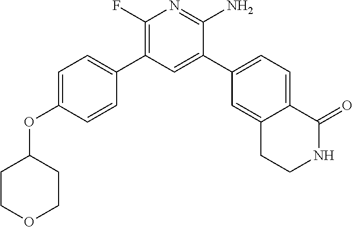 [SMILES: Nc1nc(F)c(-c2ccc(OC3CCOCC3)cc2)cc1-c1ccc2c(c1)CCNC2=O] |
| I-36 | 5-(2-amino-6-fluoro-5-(4- ((1R,5S)-3-methyl-3- azabicyclo[3.1.0]hexan-1- yl)phenyl)pyridin-3-yl)-3,3- dimethylisoindolin-1-one | 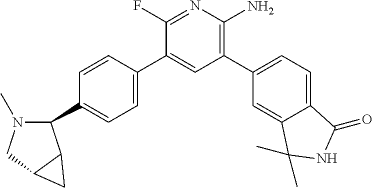 |
| I-37 | 5-(2-amino-6-fluoro-5-(4-(1- methyl-5,6-dihydro-1,2,4- triazin-4(1H)- yl)phenyl)pyridin-3-yl)-3,3- dimethylisoindolin-1-one 2,2,2-trifluoroacetate | 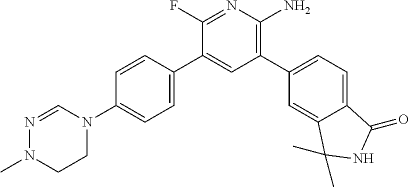 [SMILES: CN1CCN(c2ccc(-c3cc(-c4ccc5c(c4)C(C)(C)NC5=O)c(N)nc3F)cc2)C=N1] |
| I-38 | (R)-6-(2-amino-6-fluoro-5-(4- (hexahydropyrazino[2,1- c][1,4]oxazin-8(1H)- yl)phenyl)pyridin-3-yl)-3,4- dihydroisoquinolin-1(2H)- one | 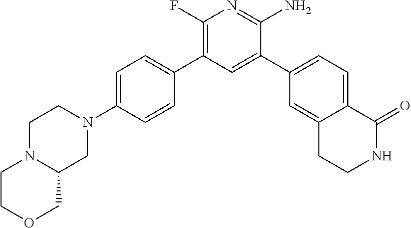 [SMILES: Nc1nc(F)c(-c2ccc(N3CCN4CCOC[C@H]4C3)cc2)cc1-c1ccc2c(c1)CCNC2=O] |
| I-39 | (S)-6-(2-amino-6-fluoro-5-(4- (hexahydropyrazino[2,1- c][1,4]oxazin-8(1H)- yl)phenyl)pyridin-3-yl)-3,4- dihydroisoquinolin-1(2H)- one | 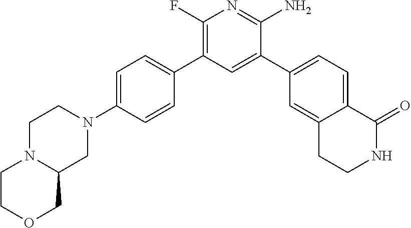 [SMILES: Nc1nc(F)c(-c2ccc(N3CCN4CCOC[C@@H]4C3)cc2)cc1-c1ccc2c(c1)CCNC2=O] |
| I-40 | 6-(2-amino-6-fluoro-5-(3- fluoro-4-(4-methylpiperazin- 1-yl)phenyl)pyridin-3-yl)-3,4- dihydroisoquinolin-1(2H)- one | 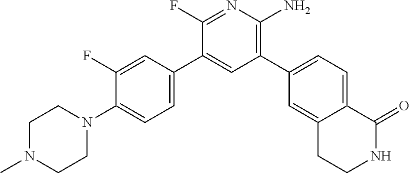 [SMILES: CN1CCN(c2ccc(-c3cc(-c4ccc5c(c4)CCNC5=O)c(N)nc3F)cc2F)CC1] |
| I-41 | 6-(2-amino-5-(4-(4- ethylpiperazin-1-yl)phenyl)- 6-fluoropyridin-3-yl)-3,4- dihydroisoquinolin-1(2H)- one | 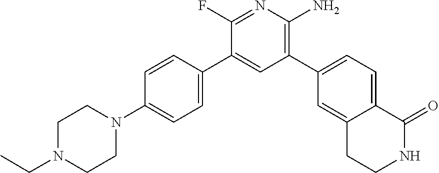 [SMILES: CCN1CCN(c2ccc(-c3cc(-c4ccc5c(c4)CCNC5=O)c(N)nc3F)cc2)CC1] |
| I-42 | 6-(2-amino-6-fluoro-5-(4-(4- isopropylpiperazin-1- yl)phenyl)pyridin-3-yl)-3,4- dihydroisoquinolin-1(2H)- one | 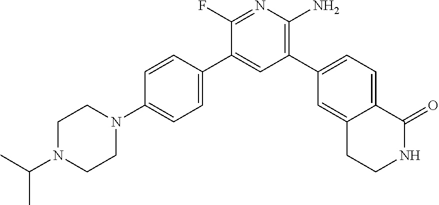 [SMILES: CC(C)N1CCN(c2ccc(-c3cc(-c4ccc5c(c4)CCNC5=O)c(N)nc3F)cc2)CC1] |
| I-43 | 6-(2-amino-5-(4-(1-(2,2- difluoroethyl)piperidin-4- yl)phenyl)-6-fluoropyridin-3- yl)-3,4-dihydroisoquinolin- 1(2H)-one | 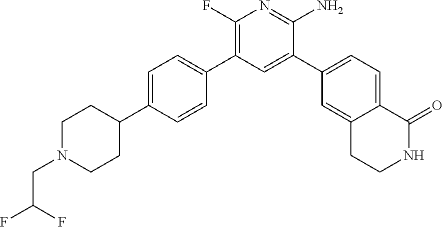 [SMILES: Nc1nc(F)c(-c2ccc(C3CCN(CC(F)F)CC3)cc2)cc1-c1ccc2c(c1)CCNC2=O] |
| I-44 | 6-(2-amino-6-fluoro-5-(4-((1- methylpiperidin-4- yl)oxy)phenyl)pyridin-3-yl)- 3,4-dihydroisoquinolin- 1(2H)-one | 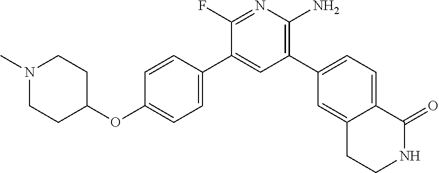 [SMILES: CN1CCC(Oc2ccc(-c3cc(-c4ccc5c(c4)CCNC5=O)c(N)nc3F)cc2)CC1] |
| I-45 | 6-(2-amino-5-(4-((2- (dimethylamino)ethyl)(methyl) amino)phenyl)-6- fluoropyridin-3-yl)-3,4- dihydroisoquinolin-1(2H)- one | 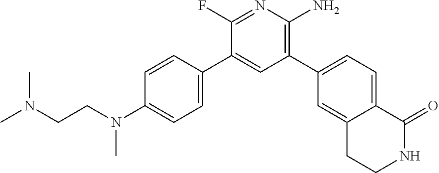 [SMILES: CN(C)CCN(C)c1ccc(-c2cc(-c3ccc4c(c3)CCNC4=O)c(N)nc2F)cc1] |
| I-46 | (R)-6-(2-amino-6-fluoro-5-(4- (2-methylpyrrolidin-1- yl)phenyl)pyridin-3-yl)-3,4- dihydroisoquinolin-1(2H)- one | 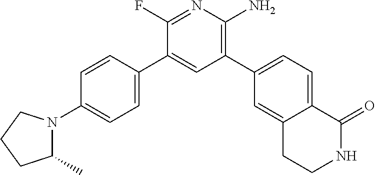 [SMILES: C[C@@H]1CCCN1c1ccc(-c2cc(-c3ccc4c(c3)CCNC4=O)c(N)nc2F)cc1] |
| I-47 | (R)-6-(2-amino-6-fluoro-5-(2- fluoro-4-(2-methylpyrrolidin- 1-yl)phenyl)pyridin-3-yl)-3,4- dihydroisoquinolin-1(2H)- one | 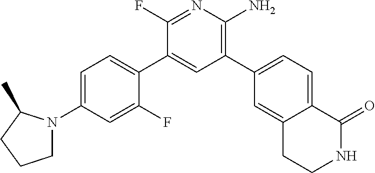 [SMILES: C[C@@H]1CCCN1c1ccc(-c2cc(-c3ccc4c(c3)CCNC4=O)c(N)nc2F)c(F)c1] |
| I-48 | (R)-6-(2-amino-6-fluoro-5-(4- (3-isopropyl-4- methylpiperazin-1- yl)phenyl)pyridin-3-yl)-3,4- dihydroisoquinolin-1(2H)- one | 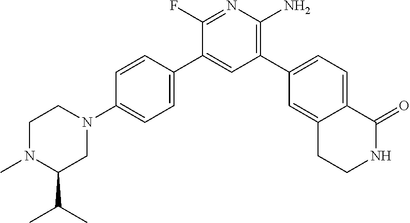 [SMILES: CC(C)[C@@H]1CN(c2ccc(-c3cc(-c4ccc5c(c4)CCNC5=O)c(N)nc3F)cc2)CCN1C] |
| I-49 | (R)-6-(2-amino-6-fluoro-5-(2- fluoro-4-(3-isoporpyl-4- methylpiperazin-1- yl)phenyl)pyridin-3-yl)-3,4- dihydroisoquinolin-1(2H)- one | 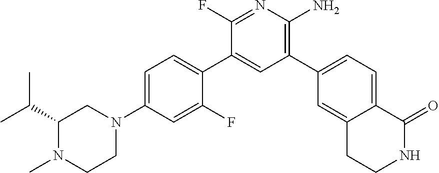 [SMILES: CC(C)[C@@H]1CN(c2ccc(-c3cc(-c4ccc5c(c4)CCNC5=O)c(N)nc3F)c(F)c2)CCN1C] |
| I-50 | (S)-6-(2-amino-6-fluoro-5-(4- (3-isopropyl-4- methylpiperazin-1- yl)phenyl)pyridin-3-yl)-3,4- dihydroisoquinolin-1(2H)- one | 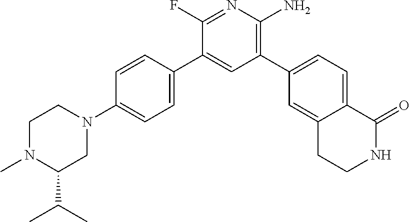 [SMILES: CC(C)[C@H]1CN(c2ccc(-c3cc(-c4ccc5c(c4)CCNC5=O)c(N)nc3F)cc2)CCN1C] |
| I-51 | (S)-6-(2-amino-6-fluoro-5-(2- fluoro-4-(3-isopropyl-4- methylpiperazin-1- yl)phenyl)pyridin-3-yl)-3,4- dihydroisoquinolin-1(2H)- one | 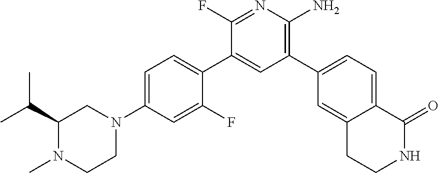 [SMILES: CC(C)[C@H]1CN(c2ccc(-c3cc(-c4ccc5c(c4)CCNC5=O)c(N)nc3F)c(F)c2)CCN1C] |
| I-52 | 6-(2-amino-5-(4-(1- ethylpiperidin-3-yl)phenyl)-6- fluoropyridin-3-yl)-3,4- dihydroisoquinolin-1(2H)- one | 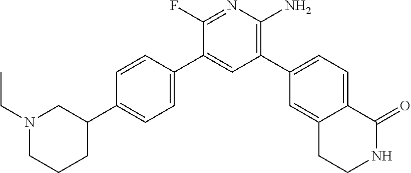 [SMILES: CCN1CCCC(c2ccc(-c3cc(-c4ccc5c(c4)CCNC5=O)c(N)nc3F)cc2)C1] |
| I-53 | 6-(2-amino-5-(4-(1- ehtylpiperidin-4-yl)phenyl)-6- fluoropyridin-3-yl)-3,4- dihydroisoquinolin-1(2H)- one | 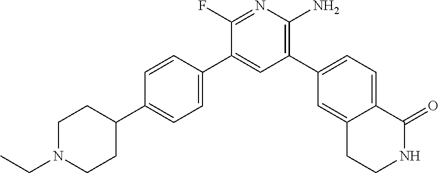 [SMILES: CCN1CCC(c2ccc(-c3cc(-c4ccc5c(c4)CCNC5=O)c(N)nc3F)cc2)CC1] |
| I-54 | 6-(2-amino-6-fluoro-5-(4-((1- (methylsulfonyl)piperidin-4- yl)oxy)phenyl)pyridin-3-yl)- 3,4-dihydroisoquinolin- 1(2H)-one | 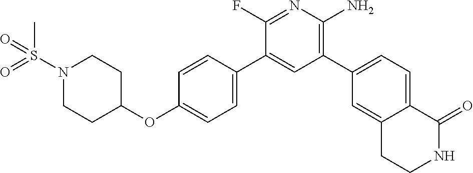 [SMILES: CS(=O)(=O)N1CCC(Oc2ccc(-c3cc(-c4ccc5c(c4)CCNC5=O)c(N)nc3F)cc2)CC1] |
| I-55 | 6-(2-amino-6-fluoro-5-(1- (tetrahydro-2H-pyran-4-yl)- 1H-pyrazol-4-yl)pyridin-3-yl)- 3,4-dihydroisoquinolin- 1(2H)-one | 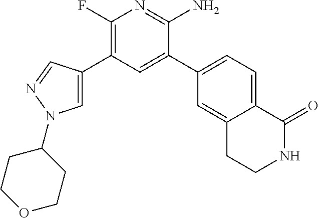 [SMILES: Nc1nc(F)c(-c2cnn(C3CCOCC3)c2)cc1-c1ccc2c(c1)CCNC2=O] |
| I-56 | tert-butyl 4-(4-(6-amino-2- fluoro-5-(1-oxo-1,2,3,4- tetrahydroisoquinolin-6- yl)pyridin-3-yl)phenyl)-3,6- dihydropyridine-1(2H)- carboxylate | 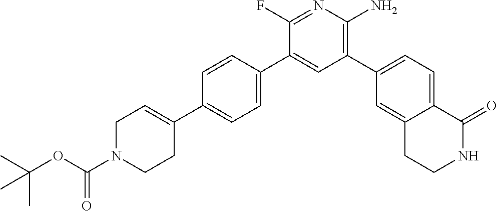 [SMILES: CC(C)(C)OC(=O)N1CC=C(c2ccc(-c3cc(-c4ccc5c(c4)CCNC5=O)c(N)nc3F)cc2)CC1] |
| I-57 | 6-(2-amino-6-fluoro-5-(4- (1,2,3,6-tetrahydropyridin-4- yl)phenyl)pyridin-3-yl)-3,4- dihydroisoquinolin-1(2H)- one 2,2,2-trifluoroacetate | 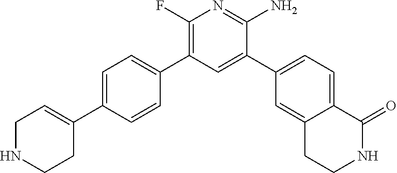 [SMILES: Nc1nc(F)c(-c2ccc(C3=CCNCC3)cc2)cc1-c1ccc2c(c1)CCNC2=O] |
| I-58 | 6-(2-amino-6-fluoro-5-(4- methyl-3,4-dihydro-2H- benzo[b][1,4]oxazin-7- yl)pyridin-3-yl)-3,4- dihydroisoquinolin-1(2H)- one | 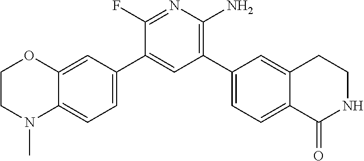 [SMILES: CN1CCOc2cc(-c3cc(-c4ccc5c(c4)CCNC5=O)c(N)nc3F)ccc21] |
| I-59 | 6-(2-amino-6-fluoro-5-(4- (pyrrolidin-1- yl)phenyl)pyridin-3-yl)-3,4- dihydroisoquinolin-1(2H)- one | 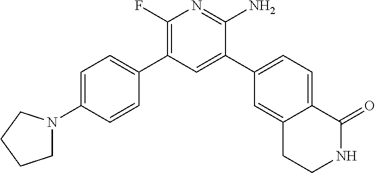 [SMILES: Nc1nc(F)c(-c2ccc(N3CCCC3)cc2)cc1-c1ccc2c(c1)CCNC2=O] |
| I-60 | 6-(2-amino-6-fluoro-5-(4- (piperidin-1- yl)phenyl)pyridin-3-yl)-3,4- dihydroisoquinolin-1(2H)- one | 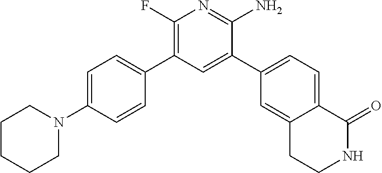 [SMILES: Nc1nc(F)c(-c2ccc(N3CCCCC3)cc2)cc1-c1ccc2c(c1)CCNC2=O] |
| I-61 | 6-(2-amino-5-(2-chloro-4-(4- methylpiperazin-1- yl)phenyl)-6-fluoropyridin-3- yl)-3,4-dihydroisoquinolin- 1(2H)-one | 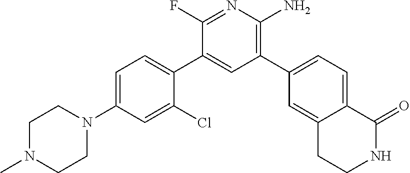 [SMILES: CN1CCN(c2ccc(-c3cc(-c4ccc5c(c4)CCNC5=O)c(N)nc3F)c(Cl)c2)CC1] |
| I-62 | 6-(2-amino-6-fluoro-5-(4- (methylsulfonyl)phenyl)pyridin- 3-yl)-3,4- dihydroisoquinolin-1(2H)- one | 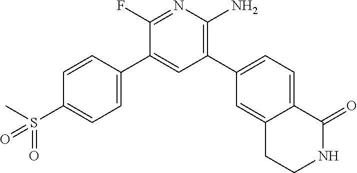 [SMILES: CS(=O)(=O)c1ccc(-c2cc(-c3ccc4c(c3)CCNC4=O)c(N)nc2F)cc1] |
| I-63 | 6-(2-amino-5-(4- (cyclopropylsulfonyl)phenyl)- 6-fluoropyridin-3-yl)-3,4- dihydroisoquinolin-1(2H)- one | 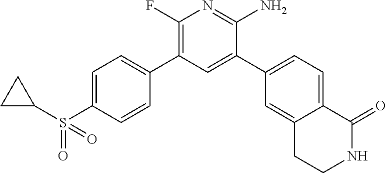 [SMILES: Nc1nc(F)c(-c2ccc(S(=O)(=O)C3CC3)cc2)cc1-c1ccc2c(c1)CCNC2=O] |
| I-64 | 4-(6-amino-2-fluoro-5-(1- oxo-1,2,3,4- tetrahydrisoquinolin-6- yl)pyridin-3-yl)-N,N- dimethylbenzenesulfonamide | 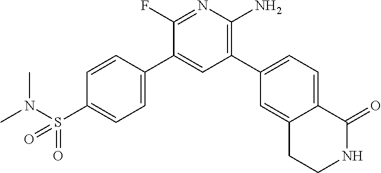 [SMILES: CN(C)S(=O)(=O)c1ccc(-c2cc(-c3ccc4c(c3)CCNC4=O)c(N)nc2F)cc1] |
| I-65 | 4-(6-amino-2-fluoro-5-(1- oxo-1,2,3,4- tetrahydroisoquinolin-6- yl)pyridin-3-yl)-N,N- diethylbenzenesulfonamide | 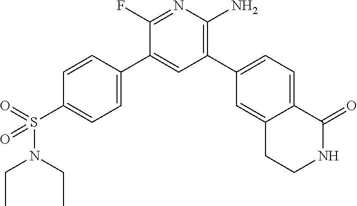 [SMILES: CCN(CC)S(=O)(=O)c1ccc(-c2cc(-c3ccc4c(c3)CCNC4=O)c(N)nc2F)cc1] |
| I-66 | 6-(2-amino-6-fluoro-5-(4- ((1R,5S)-3-(tetrahydro-2H- pyrazn-4-yl)-3- azabicyclo[3.1.0]hexan-1- yl)phenyl)pyridin-3-yl)-3,4- dihydroisoquinolin-1(2H)- one | 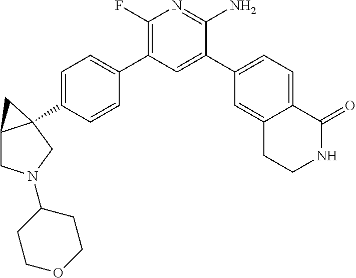 [SMILES: Nc1nc(F)c(-c2ccc([C@@]34C[C@@H]3CN(C3CCOCC3)C4)cc2)cc1-c1ccc2c(c1)CCNC2=O] |
| I-67 | 7-(2-amino-5-(4-(4- ethylpiperazin-1-yl)phenyl)- 6-fluoropyridin-3-yl)-2,3,4,5- tetrahydro-1H- benzo[c]azepin-1-one | 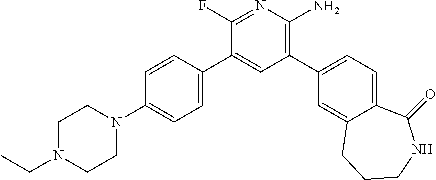 [SMILES: CCN1CCN(c2ccc(-c3cc(-c4ccc5c(c4)CCCNC5=O)c(N)nc3F)cc2)CC1] |
| I-68 | 6-(2-amino-6-fluoro-5-(4-(4- methylpiperazin-1-yl)-3- (trifluoromethyl)phenyl)pyridin- 3-yl)-3,4- dihydroisoquinolin-1(2H)- one | 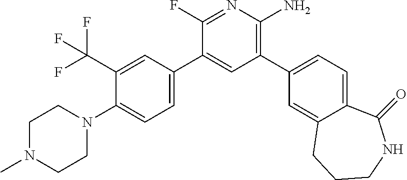 [SMILES: CN1CCN(c2ccc(-c3cc(-c4ccc5c(c4)CCCNC5=O)c(N)nc3F)cc2C(F)(F)F)CC1] |
| I-69 | 6-(2-amino-5-(4-(4- ethylpiperazin-1-yl)phenyl)- 6-fluoropyridin-3-yl)-4,4- dimethyl-3,4- dihydroisoquinolin-1(2H)- one | 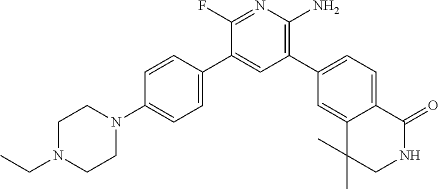 [SMILES: CCN1CCN(c2ccc(-c3cc(-c4ccc5c(c4)C(C)(C)CNC5=O)c(N)nc3F)cc2)CC1] |
| I-70 | 6-(2-amino-5-(4-(4- ethylpiperazin-1-yl)phenyl)- 6-fluoropyridin-3-yl)-3,3- dimethyl-3,4- dihydroisoquinolin-1(2H)- one 2,2,2-trifluoroacetate | 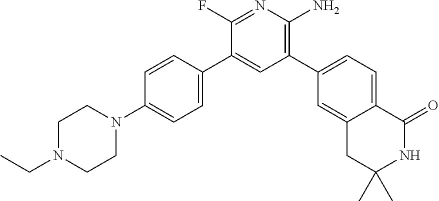 [SMILES: CCN1CCN(c2ccc(-c3cc(-c4ccc5c(c4)CC(C)(C)NC5=O)c(N)nc3F)cc2)CC1] |
| I-71 | 6-(2-amino-5-(4-((1R,5S)-3- ethyl-3- azabicyclo[3.1.0]hexan-1- yl)phenyl)-6-fluoropyridin-3- yl)-3,4-dihydroisoquinolin- 1(2H)-one | 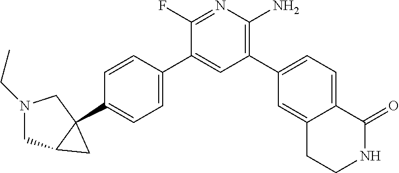 [SMILES: CCN1C[C@H]2C[C@@]2(c2ccc(-c3cc(-c4ccc5c(c4)CCNC5=O)c(N)nc3F)cc2)C1] |
| I-72 | 6-(2-amino-5-(4-((1R,5S)-3- (2,2-difluoroethyl)-3- azabicyclo[3.1.0]hexan-1- yl)phenyl)-6-fluoropyridin-3- yl)-3,4-dihydroisoquinolin- 1(2H)-one | 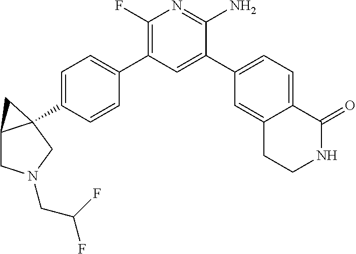 [SMILES: Nc1nc(F)c(-c2ccc([C@@]34C[C@@H]3CN(CC(F)F)C4)cc2)cc1-c1ccc2c(c1)CCNC2=O] |
| I-73 | 6-(2-amino-5-(4-(1-(2,2- difluoroethyl)-1,2,3,6- tetrahydropyridin-4- yl)phenyl)-6-fluoropyridin-3- yl)-3,4-dihydroisoquinolin- 1(2H)-one | 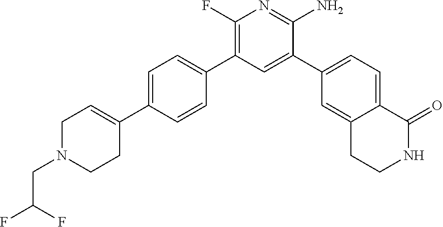 [SMILES: Nc1nc(F)c(-c2ccc(C3=CCN(CC(F)F)CC3)cc2)cc1-c1ccc2c(c1)CCNC2=O] |
| I-74 | 6-(2-amino-6-chloro-5-(4-(4- methylpiperazin-1- yl)phenyl)pyridin-3-yl)-3,4- dihydroisoquinolin-1(2H)- one | 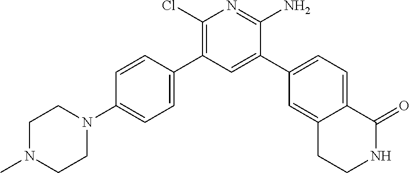 [SMILES: CN1CCN(c2ccc(-c3cc(-c4ccc5c(c4)CCNC5=O)c(N)nc3Cl)cc2)CC1] |
| I-75 | 6-(2-amino-6-chloro-5-(4-(4- ethylpiperazin-1- yl)phenyl)pyridin-3-yl)-3,4- dihydroisoquinolin-1(2H)- one | 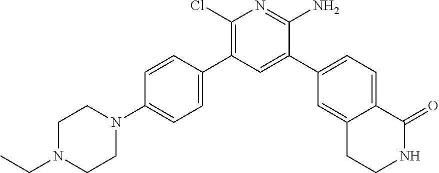 [SMILES: CCN1CCN(c2ccc(-c3cc(-c4ccc5c(c4)CCNC5=O)c(N)nc3Cl)cc2)CC1] |
| I-76 | 6-(2-amino-5-(4-(1-ethyl- 1,2,3,6-tetrahydropyridin-4- yl)phenyl)-6-fluoropyridin-3- yl)-3,4-dihydroisoquinolin- 1(2H)-one | 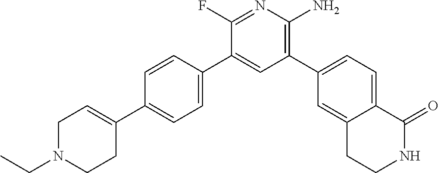 [SMILES: CCN1CC=C(c2ccc(-c3cc(-c4ccc5c(c4)CCNC5=O)c(N)nc3F)cc2)CC1] |
| I-77 | (S)-6-(2-amino-6-fluoro-5-(4- (2- isopropylmorpholino)phenyl) pyridin-3-yl)-3,4-dihydro-2,7- naphthyridin-1(2H)-one 2,2,2-trifluoroacetate | 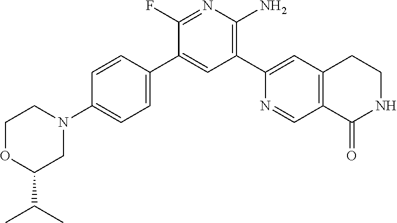 [SMILES: CC(C)[C@H]1CN(c2ccc(-c3cc(-c4cc5c(cn4)C(=O)NCC5)c(N)nc3F)cc2)CCO1] |
| I-78 | 6-(2-amino-5-(4-(1-(2,2- difluoroethyl)piperidin-3- yl)phenyl)-6-fluoropyridin-3- yl)-3,4-dihydroisoquinolin- 1(2H)-one | 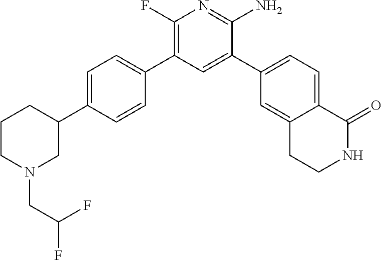 [SMILES: Nc1nc(F)c(-c2ccc(C3CCCN(CC(F)F)C3)cc2)cc1-c1ccc2c(c1)CCNC2=O] |
| I-79 | 6-(2-amino-6-fluoro-5-(4-(1- isopropylpiperidin-4- yl)phenyl)pyridin-3-yl)-3,4- dihydroisoquinolin-1(2H)- one | 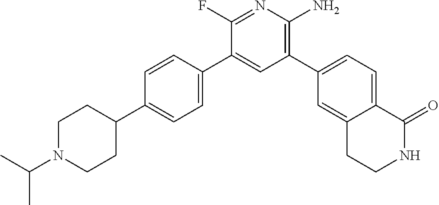 [SMILES: CC(C)N1CCC(c2ccc(-c3cc(-c4ccc5c(c4)CCNC5=O)c(N)nc3F)cc2)CC1] |
| I-80 | 6-(2-amino-6-fluoro-5-(4-(1- isopropylpiperidin-3- yl)phenyl)pyridin-3-yl)-3,4- dihydroisoquinolin-1(2H)- one | 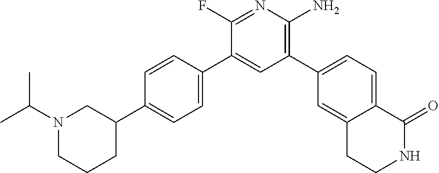 [SMILES: CC(C)N1CCCC(c2ccc(-c3cc(-c4ccc5c(c4)CCNC5=O)c(N)nc3F)cc2)C1] |
| I-81 | 6-(2-amino-6-fluoro-5-(4-(1- isopropyl-1,2,3,6- tetrahydropyridin-4- yl)phenyl)pyridin-3-yl)-3,4- dihydroisoquinolin-1(2H)- one | 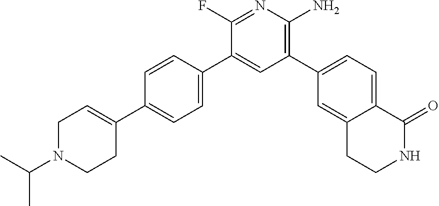 [SMILES: CC(C)N1CC=C(c2ccc(-c3cc(-c4ccc5c(c4)CCNC5=O)c(N)nc3F)cc2)CC1] |
| I-82 | 6-(2-amino-6-fluoro-5-(4-(1- (2-hydroxyethyl)piperidin-3- yl)phenyl)pyridin-3-yl)-3,4- dihydroisoquinolin-1(2H)- one | 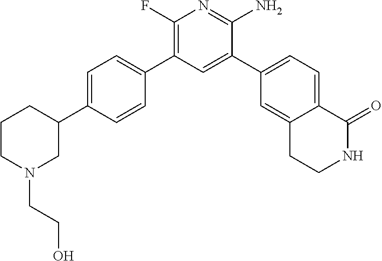 [SMILES: Nc1nc(F)c(-c2ccc(C3CCCN(CCO)C3)cc2)cc1-c1ccc2c(c1)CCNC2=O] |
| I-83 | tert-butyl 4-((4-(6-amino-2- fluoro-5-(1-oxo-1,2,3,4- tetrahydroisoquinolin-6- yl)pyridin-3- yl)phenyl)sulfonyl)piperidine- 1-carboxylate | 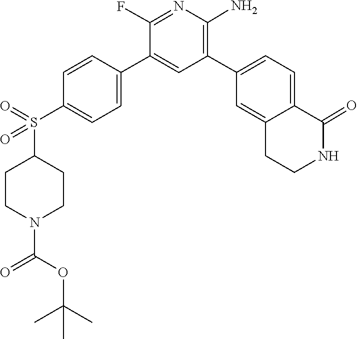 [SMILES: CC(C)(C)OC(=O)N1CCC(S(=O)(=O)c2ccc(-c3cc(-c4ccc5c(c4)CCNC5=O)c(N)nc3F)cc2)CC1] |
| I-84 | 6-(2-amino-5-(4-(azetidin-3- ylsulfonyl)phenyl)-6- fluoropyridin-3-yl)-3,4- dihydroisoquinolin-1(2H)- one | 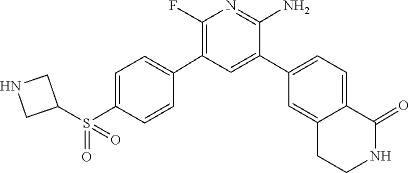 [SMILES: Nc1nc(F)c(-c2ccc(S(=O)(=O)C3CNC3)cc2)cc1-c1ccc2c(c1)CCNC2=O] |
| I-85 | 6-(2-amino-5-(4- (ethylsulfonimidoyl)phenyl)- 6-fluoropyridin-3-yl)-3,4- dihydroisoquinolin-1(2H)- one | 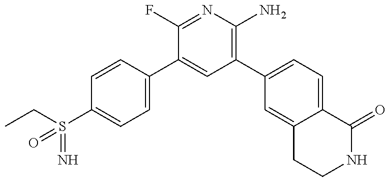 [SMILES: CCS(=N)(=O)c1ccc(-c2cc(-c3ccc4c(c3)CCNC4=O)c(N)nc2F)cc1] |
| I-86 | 6-(2-amino-6-fluoro-5-(4-(S- methylsulfonimidoyl)phenyl) pyridin-3-yl)-3,4- dihydroisoquinolin-1(2H)- one | 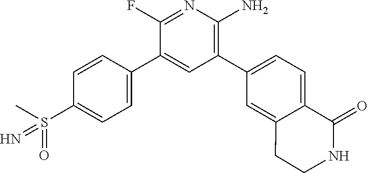 [SMILES: CS(=N)(=O)c1ccc(-c2cc(-c3ccc4c(c3)CCNC4=O)c(N)nc2F)cc1] |
| I-87 | 6-(2-amino-6-fluoro-5-(4- ((trifluoromethyl)sulfonyl)phe- nyl)pyridin-3-yl)-3,4- dihydroisoquinolin-1(2H)- one | 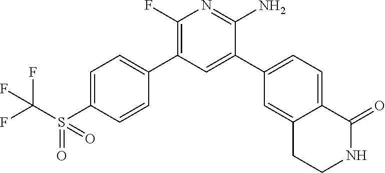 [SMILES: Nc1nc(F)c(-c2ccc(S(=O)(=O)C(F)(F)F)cc2)cc1-c1ccc2c(c1)CCNC2=O] |
| I-88 | 6-(2-amino-5-(4-(3,4- dimethylpiperazin-1- yl)phenyl)-6-fluoropyridin-3- yl)-3,4-dihydroisoquinolin- 1(2H)-one | 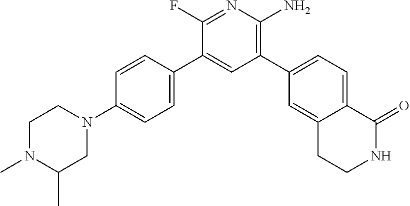 [SMILES: CC1CN(c2ccc(-c3cc(-c4ccc5c(c4)CCNC5=O)c(N)nc3F)cc2)CCN1C] |
| I-89 | 6-(2-amino-6-fluoro-5-(4-(1- (2,2,2-trifluoroethyl)piperidin- 3-yl)phenyl)pyridin-3-yl)-3,4- dihydroisoquinolin-1(2H)- one | 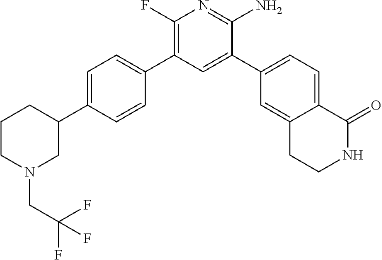 [SMILES: Nc1nc(F)c(-c2ccc(C3CCCN(CC(F)(F)F)C3)cc2)cc1-c1ccc2c(c1)CCNC2=O] |
| I-90 | 6-(2-amino-6-fluoro-5-(4-(4- (2,2,2- trifluoroethyl)morpholin-2- yl)phenyl)pyridin-3-yl)-3,4- dihydroisoquinolin-1(2H)- one | 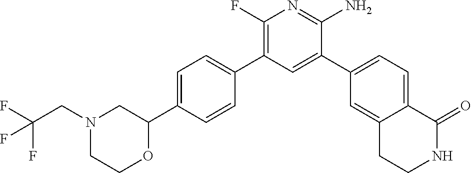 [SMILES: Nc1nc(F)c(-c2ccc(C3CN(CC(F)(F)F)CCO3)cc2)cc1-c1ccc2c(c1)CCNC2=O] |
| I-91 | 6-(2-amino-6-fluoro-5-(4- ((3aR,6aS)-tetrahydro-1H- furo[3,4-c]pyrrol-5(3H)- yl)phenyl)pyridin-3-yl)-3,4- dihydroisoquinolin-1(2H)- one | 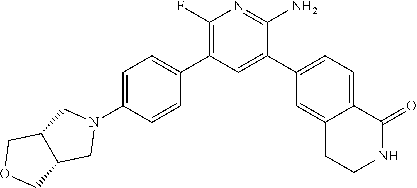 [SMILES: Nc1nc(F)c(-c2ccc(N3C[C@H]4COC[C@H]4C3)cc2)cc1-c1ccc2c(c1)CCNC2=O] |
| I-92 | 6-(2-amino-6-fluoro-5-(4- ((3aS,6aS)-hexahydro-5H- furo[2,3-c]pyrrol-5- yl)phenyl)pyridin-3-yl)-3,4- dihydroisoquinolin-1(2H)- one | 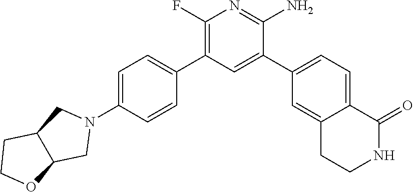 [SMILES: Nc1nc(F)c(-c2ccc(N3C[C@@H]4CCO[C@@H]4C3)cc2)cc1-c1ccc2c(c1)CCNC2=O] |
| I-93 | 6-(2-amino-6-fluoro-5-(4-(1- (2,2,2-trifluoroethyl)piperidin- 2-yl)phenyl)pyridin-3-yl)-3,4- dihydroisoquinolin-1(2H)- one | 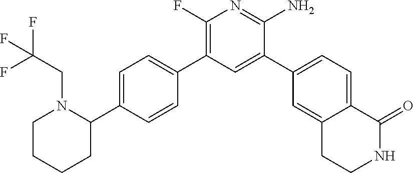 [SMILES: Nc1nc(F)c(-c2ccc(C3CCCCN3CC(F)(F)F)cc2)cc1-c1ccc2c(c1)CCNC2=O] |
| I-94 | (S)-6-(2-amino-6-fluoro-5-(4- (piperidin-3- yloxy)phenyl)pyridin-3-yl)- 3,4-dihydroisoquinolin- 1(2H)-one | 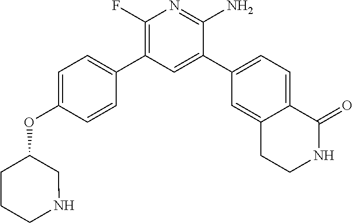 [SMILES: Nc1nc(F)c(-c2ccc(O[C@H]3CCCNC3)cc2)cc1-c1ccc2c(c1)CCNC2=O] |
| I-95 | 6-(2-amino-6-fluoro-5-(4-(4- isopropylpiperazin-1- yl)phenyl)pyridin-3-yl)-7- fluoro-3,4- dihydroisoquinolin-1(2H)- one | 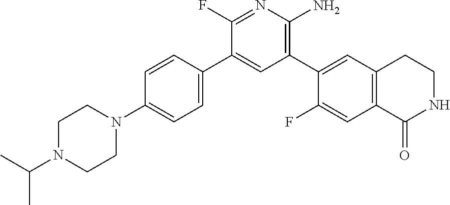 [SMILES: CC(C)N1CCN(c2ccc(-c3cc(-c4cc5c(cc4F)C(=O)NCC5)c(N)nc3F)cc2)CC1] |
| I-96 | 6-(2-amino-6-fluoro-5-(4- ((3R,5S)-3,4,5- trimethylpiperazin-1- yl)phenyl)pyridin-3-yl)-3,4- dihydroisoquinolin-1(2H)- one | 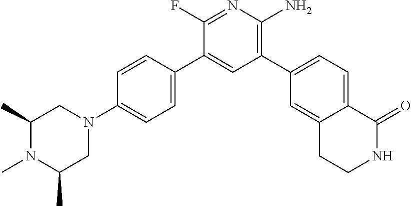 [SMILES: C[C@@H]1CN(c2ccc(-c3cc(-c4ccc5c(c4)CCNC5=O)c(N)nc3F)cc2)C[C@H](C)N1C] |
| I-97 | 6-(2-amino-6-fluoro-5-(4- ((3S,5S)-3,4,5- trimethylpiperazin-1- yl)phenyl)pyridin-3-yl)-3,4- dihydroisoquinolin-1(2H)- one | 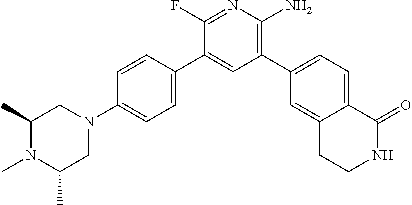 [SMILES: C[C@H]1CN(c2ccc(-c3cc(-c4ccc5c(c4)CCNC5=O)c(N)nc3F)cc2)C[C@H](C)N1C] |
| I-98 | 6-(2-amino-6-fluoro-5-(4- (morpholinosulfonyl)phenyl) pyridin-3-yl)-3,4- dihydroisoquinolin-1(2H)- one | 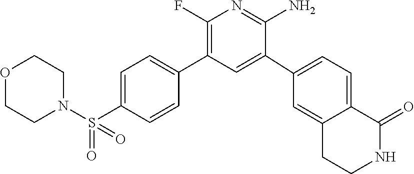 [SMILES: Nc1nc(F)c(-c2ccc(S(=O)(=O)N3CCOCC3)cc2)cc1-c1ccc2c(c1)CCNC2=O] |
| I-99 | 6-(2-amino-6-fluoro-5-(4- (pyrrolidin-1- ylsulfonyl)phenyl)pyridin-3- yl)-3,4-dihydroisoquinolin- 1(2H)-one | 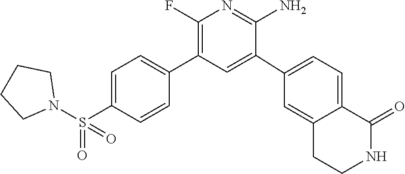 [SMILES: Nc1nc(F)c(-c2ccc(S(=O)(=O)N3CCCC3)cc2)cc1-c1ccc2c(c1)CCNC2=O] |
| I-100 | 6-(2-amino-5-(4-(1- ethylpiperidin-2-yl)phenyl)-6- fluoropyridin-3-yl)-3,4- dihydroisoquinolin-1(2H)- one | 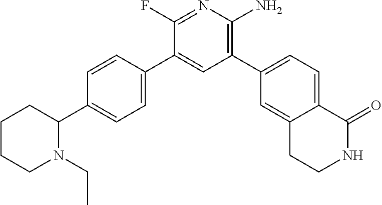 [SMILES: CCN1CCCCC1c1ccc(-c2cc(-c3ccc4c(c3)CCNC4=O)c(N)nc2F)cc1] |
| I-101 | 6-(2-amino-5-(4-(1-(2,2- difluoroethyl)piperidin-2- yl)phenyl)-6-fluoropyridin-3- yl)-3,4-dihydroisoquinolin- 1(2H)-one | 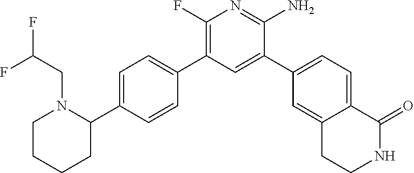 [SMILES: Nc1nc(F)c(-c2ccc(C3CCCCN3CC(F)F)cc2)cc1-c1ccc2c(c1)CCNC2=O] |
| I-102 | 6-(2-amino-5-(4-(4- ethylmorpholin-2-yl)phenyl)- 6-fluoropyridin-3-yl)-3,4- dihydroisoquinolin-1(2H)- one | 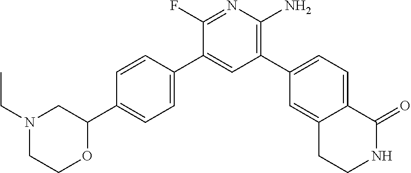 [SMILES: CCN1CCOC(c2ccc(-c3cc(-c4ccc5c(c4)CCNC5=O)c(N)nc3F)cc2)C1] |
| I-103 | 6-(2-amino-5-(4-(4-(2,2- difluoroethyl)morpholin-2- yl)phenyl)-6-fluoropyriidn-3- yl)-3,4-dihydroisoquinolin- 1(2H)-one | 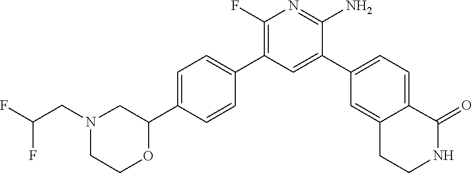 [SMILES: Nc1nc(F)c(-c2ccc(C3CN(CC(F)F)CCO3)cc2)cc1-c1ccc2c(c1)CCNC2=O] |
| I-104 | 6-(2-amino-6-fluoro-5-(4-(1- isopropylpiperidin-2- yl)phenyl)pyridin-3-yl)-3,4- dihydroisoquinolin-1(2H)- one | 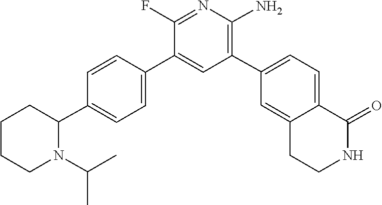 [SMILES: CC(C)N1CCCCC1c1ccc(-c2cc(-c3ccc4c(c3)CCNC4=O)c(N)nc2F)cc1] |
| I-105 | 6-(2-amino-6-fluoro-5-(4-(4- isopropylmorpholin-2- yl)phenyl)pyridin-3-yl)-3,4- dihydroisoquinolin-1(2H)- one | 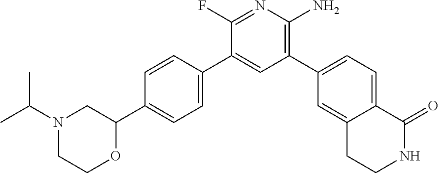 [SMILES: CC(C)N1CCOC(c2ccc(-c3cc(-c4ccc5c(c4)CCNC5=O)c(N)nc3F)cc2)C1] |
| I-106 | 6-(2-amino-6-fluoro-5-(4- ((1S,5R)-3-methyl-3- azabicyclo[3.1.0]hexan-1- yl)phenyl)pyriidn-3-yl)-3,4- dihydroisoquinolin-1(2H)- one | 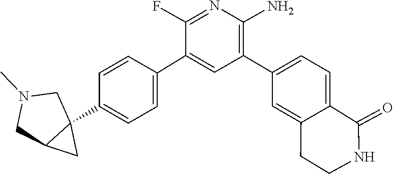 [SMILES: CN1C[C@@H]2C[C@]2(c2ccc(-c3cc(-c4ccc5c(c4)CCNC5=O)c(N)nc3F)cc2)C1] |
| I-107 | 6-(2-amino-6-fluoro-5-(4-(1- (2-methoxyethyl)piperidin-4- yl)phenyl)pyridin-3-yl)-3,4- dihydroisoquinolin-1(2H)- one | 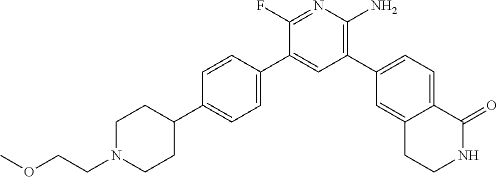 [SMILES: COCCN1CCC(c2ccc(-c3cc(-c4ccc5c(c4)CCNC5=O)c(N)nc3F)cc2)CC1] |
| I-108 | (R)-6-(2-amino-6-fluoro-5-(2- fluoro-4-(3- methylmorpholino)phenyl)pyr- ridin-3-yl)-3,4- dihydroisoquinolin-1(2H)- one | 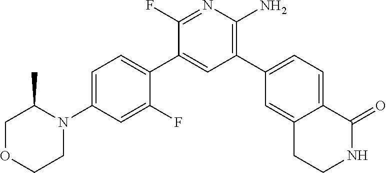 [SMILES: C[C@@H]1COCCN1c1ccc(-c2cc(-c3ccc4c(c3)CCNC4=O)c(N)nc2F)c(F)c1] |
| I-109 | 6-(2-amino-5-(4-(2,5- dihydro-1H-pyrrol-3- yl)phenyl)-6-fluoropyridin-3- yl)-3,4-dihydroisoquinolin- 1(2H)-one 2,2,2- trifluoroacetate | 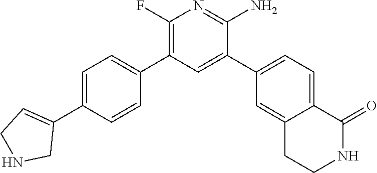 [SMILES: Nc1nc(F)c(-c2ccc(C3=CCNC3)cc2)cc1-c1ccc2c(c1)CCNC2=O] |
| I-110 | 6-(2-amino-6-fluoro-5-(4- ((1R,5S)-3-isopropyl-3- azabicyclo[3.1.0]hexan-1- yl)phenyl)pyridin-3-yl)-3,4- dihydroisoquinolin-1(2H)- one | 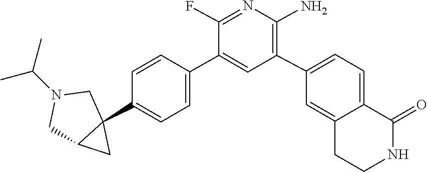 [SMILES: CC(C)N1C[C@H]2C[C@@]2(c2ccc(-c3cc(-c4ccc5c(c4)CCNC5=O)c(N)nc3F)cc2)C1] |
| I-111 | 6-(2-amino-6-fluoro-5-(4- ((1S,5R)-3-isoporpyl-3- azabicyclo[3.1.0]hexan-1- yl)phenyl)pyridin-3-yl)-3,4- dihydroisoquinolin-1(2H)- one 2,2,2-trifluoroacetate | 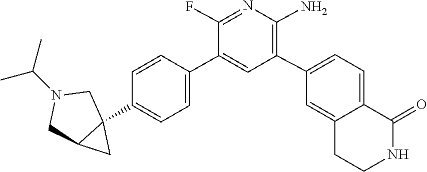 [SMILES: CC(C)N1C[C@@H]2C[C@]2(c2ccc(-c3cc(-c4ccc5c(c4)CCNC5=O)c(N)nc3F)cc2)C1] |
| I-112 | (R)-6-(2-amino-6-fluoro-5-(4- (3-fluoropyrrolidin-1- yl)phenyl)pyriidn-3-yl)-3,4- dihydroisoquinolin-1(2H)- one | 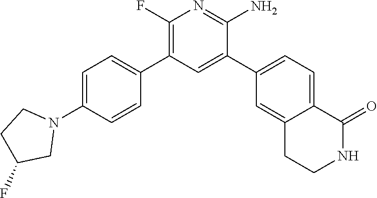 [SMILES: Nc1nc(F)c(-c2ccc(N3CC[C@@H](F)C3)cc2)cc1-c1ccc2c(c1)CCNC2=O] |
| I-113 | (S)-6-(2-amino-6-fluoro-5-(4- (3-fluoropyrrolidin-1- yl)phenyl)pyridin-3-yl)-3,4- dihydroisoquinolin-1(2H)- one | 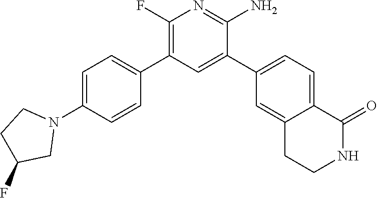 [SMILES: Nc1nc(F)c(-c2ccc(N3CC[C@H](F)C3)cc2)cc1-c1ccc2c(c1)CCNC2=O] |
| I-114 | (R)-6-(2-amino-6-fluoro-5-(4- (3-methoxypyrrolidin-1- yl)phenyl)pyridin-3-yl)-3,4- dihydroisoquinolin-1(2H)- one | 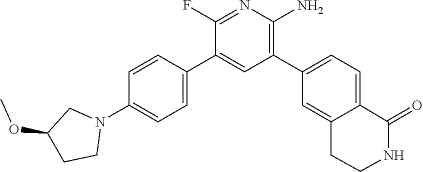 [SMILES: CO[C@@H]1CCN(c2ccc(-c3cc(-c4ccc5c(c4)CCNC5=O)c(N)nc3F)cc2)C1] |
| I-115 | (S)-6-(2-amino-6-fluoro-5-(4- (3-methoxypyrrolidin-1- yl)phenyl)pyridin-3-yl)-3,4- dihydroisoquinolin-1(2H)- one | 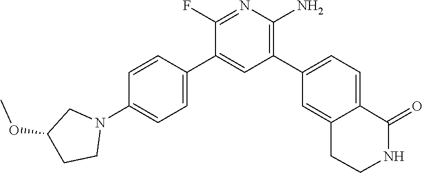 [SMILES: CO[C@H]1CCN(c2ccc(-c3cc(-c4ccc5c(c4)CCNC5=O)c(N)nc3F)cc2)C1] |
| I-116 | (R)-6-(2-amino-6-fluoro-5-(4- (3-(methylsulfonyl)pyrrolidin- 1-yl)phenyl)pyridin-3-yl)-3,4- dihydroisoquinolin-1(2H)- one | 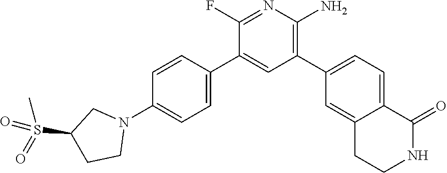 [SMILES: CS(=O)(=O)[C@@H]1CCN(c2ccc(-c3cc(-c4ccc5c(c4)CCNC5=O)c(N)nc3F)cc2)C1] |
| I-117 | (S)-6-(2-amino-6-fluoro-5-(4- (3-(methylsulfonyl)pyrrolidin- 1-yl)phenyl)pyridin-3-yl)-3,4- dihydroisoquinolin-1(2H)- one | 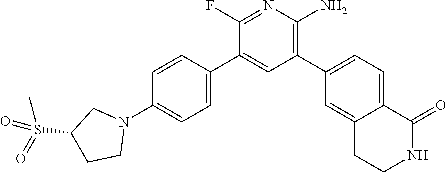 [SMILES: CS(=O)(=O)[C@H]1CCN(c2ccc(-c3cc(-c4ccc5c(c4)CCNC5=O)c(N)nc3F)cc2)C1] |
| I-118 | 6-(2-amino-6-fluoro-5-(4-(4- methyl-5,6-dihydro-1,2,4- triazin-1(4H)- yl)phenyl)pyridin-3-yl)-3,4- dihydroisoquinolin-1(2H)- one | 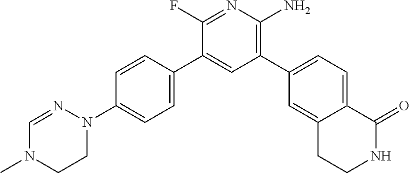 [SMILES: CN1C=NN(c2ccc(-c3cc(-c4ccc5c(c4)CCNC5=O)c(N)nc3F)cc2)CC1] |
| I-119 | 6-(2-amino-6-fluoro-5-(4-(1- (2-hydroxyethyl)piperidin-4- yl)phenyl)pyridin-3-yl)-3,4- dihydroisoquinolin-1(2H)- one | 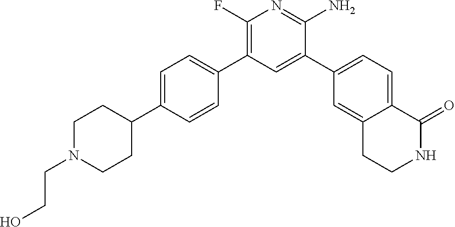 [SMILES: Nc1nc(F)c(-c2ccc(C3CCN(CCO)CC3)cc2)cc1-c1ccc2c(c1)CCNC2=O] |
| I-120 | 6-(2-amino-5-(4-(1-ethyl-2,5- dihydro-1H-pyrrol-3- yl)phenyl)-6-fluoropyridin-3- yl)-3,4-dihydroisoquinolin- 1(2H)-one | 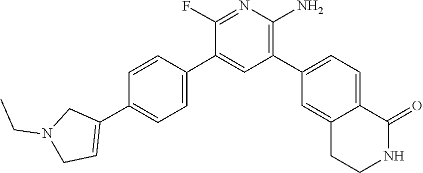 [SMILES: CCN1CC=C(c2ccc(-c3cc(-c4ccc5c(c4)CCNC5=O)c(N)nc3F)cc2)C1] |
| I-121 | 6-(2-amino-6-fluoro-5-(4-(1- isopropyl-2,5-dihydro-1H- pyrrol-3-yl)phenyl)pyridin-3- yl)-3,4-dihydroisoquinolin- 1(2H)-one | 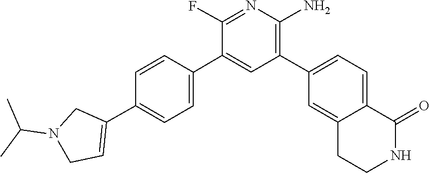 [SMILES: CC(C)N1CC=C(c2ccc(-c3cc(-c4ccc5c(c4)CCNC5=O)c(N)nc3F)cc2)C1] |
| I-122 | 6-(2-amino-6-fluoro-5-(4-(1- (2-methoxyethyl)piperidin-3- yl)phenyl)pyridin-3-yl)-3,4- dihydroisoquinolin-1(2H)- one | 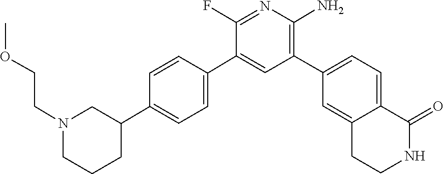 [SMILES: COCCN1CCCC(c2ccc(-c3cc(-c4ccc5c(c4)CCNC5=O)c(N)nc3F)cc2)C1] |
| I-123 | 6-(2-amino-6-fluoro-5-(4-(1- (2-hydroxyethyl)-2,5- dihydro-1H-pyrrol-3- yl)phenyl)pyridin-3-yl)-3,4- dihydroisoquinolin-1(2H)- one | 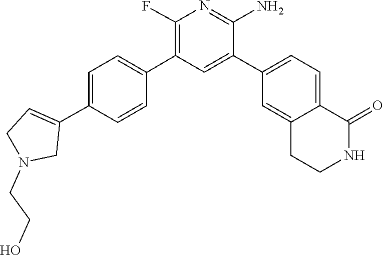 [SMILES: Nc1nc(F)c(-c2ccc(C3=CCN(CCO)C3)cc2)cc1-c1ccc2c(c1)CCNC2=O] |
| I-124 | 6-(2-amino-6-fluoro-5-(4-(1- (2-methoxyethyl)-2,5- dihydro-1H-pyrrol-3- yl)phenyl)pyridin-3-yl)-3,4- dihydroisoquinolin-1(2H)- one | 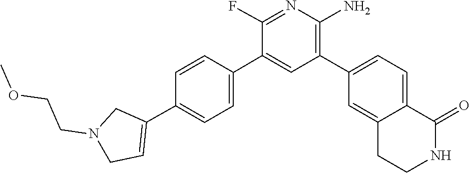 [SMILES: COCCN1CC=C(c2ccc(-c3cc(-c4ccc5c(c4)CCNC5=O)c(N)nc3F)cc2)C1] |
| I-125 | 6-(2-amino-6-fluoro-5-(4- ((1R,5S)-3-(2-hydroxy-2- methylpropyl)-3- azabicyclo[3.1.0]hexan-1- yl)phenyl)pyridin-3-yl)-3,4- dihydroisoquinolin-1(2H)- one | 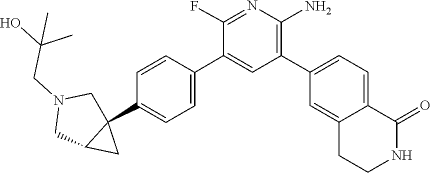 [SMILES: CC(C)(O)CN1C[C@H]2C[C@@]2(c2ccc(-c3cc(-c4ccc5c(c4)CCNC5=O)c(N)nc3F)cc2)C1] |
| I-126 | 6-(2-amino-6-fluoro-5-(4- ((1S,4R)-3-(2-hydroxy-2- methylpropyl)-3- azabicyclo[3.1.0]hexan-1- yl)phenyl)pyridin-3-yl)-3,4- dihydroisoquinolin-1(2H)- one | 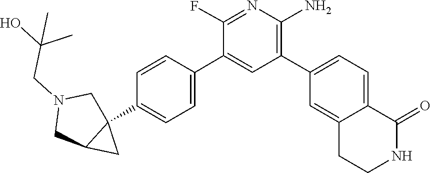 [SMILES: CC(C)(O)CN1C[C@@H]2C[C@]2(c2ccc(-c3cc(-c4ccc5c(c4)CCNC5=O)c(N)nc3F)cc2)C1] |
| I-128 | 6-(2-amino-6-fluoro-5-(4- (piperidin-4- ylsulfonl)phenyl)pyridin-3- yl)-3,4-dihydroisoquinolin- 1(2H)-one | 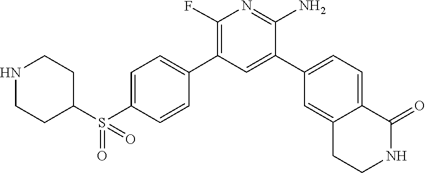 [SMILES: Nc1nc(F)c(-c2ccc(S(=O)(=O)C3CCNCC3)cc2)cc1-c1ccc2c(c1)CCNC2=O] |
| I-129 | 6-(2-amino-6-fluoro-5-(1-(1- methylpiperidin-4-yl)-1H- pyrazol-4-yl)pyridin-3-yl)-7- fluoro-3,4- dihydroisoquinolin-1(2H)- one | 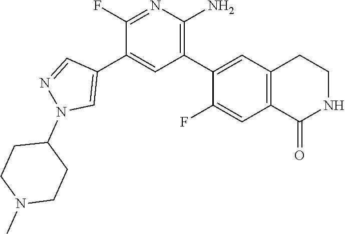 [SMILES: CN1CCC(n2cc(-c3cc(-c4cc5c(cc4F)C(=O)NCC5)c(N)nc3F)cn2)CC1] |
| I-130 | (S)-6-(2-amino-6-fluoro-5-(4- (2-isopropylmorpholino)-3- (trifluoromethyl)phenyl)pyridin- 3-yl)-3,4- dihydroisoquinolin-1(2H)- one | 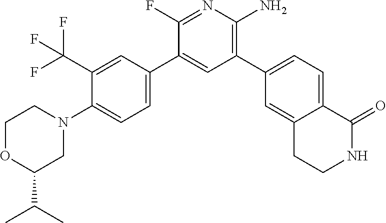 [SMILES: CC(C)[C@H]1CN(c2ccc(-c3cc(-c4ccc5c(c4)CCNC5=O)c(N)nc3F)cc2C(F)(F)F)CCO1] |
| I-131 | 6-(2-amino-5-(4-(4- ethylmorpholin-3-yl)phenyl)- 6-fluoropyridin-3-yl)-3,4- dihydroisoquinolin-1(2H)- one | 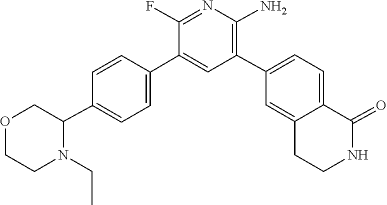 [SMILES: CCN1CCOCC1c1ccc(-c2cc(-c3ccc4c(c3)CCNC4=O)c(N)nc2F)cc1] |
| I-132 | 6-(2-amino-6-fluoro-5-(4-(4- isopropylmorpholin-3- yl)phenyl)pyridin-3-yl)-3,4- dihydroisoquinolin-1(2H)- one | 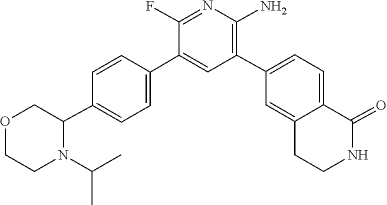 [SMILES: CC(C)N1CCOCC1c1ccc(-c2cc(-c3ccc4c(c3)CCNC4=O)c(N)nc2F)cc1] |
| I-133 | 6-(2-amino-5-(4-(4-(2,2- difluoroethyl)morpholin-3- yl)phenyl)-6-fluoropyridin-3- yl)-3,4-dihydroisoquinolin- 1(2H)-one | 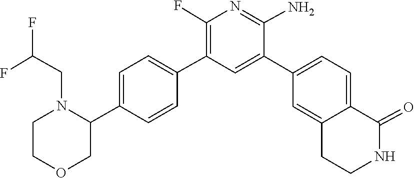 [SMILES: Nc1nc(F)c(-c2ccc(C3COCCN3CC(F)F)cc2)cc1-c1ccc2c(c1)CCNC2=O] |
| I-134 | 6-(2-amino-6-fluoro-5-(4-(4- (2-methoxyethyl)morpholin- 3-yl)phenyl)pyridin-3-yl)-3,4- dihydroisoquinolin-1(2H)- one | 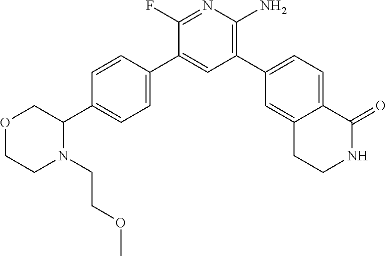 [SMILES: COCCN1CCOCC1c1ccc(-c2cc(-c3ccc4c(c3)CCNC4=O)c(N)nc2F)cc1] |
| I-135 | 6-(5-(4-((1R,5S)-3- azabicyclo[3.1.0]hexan-1- yl)phenyl)-2-amino-6- fluoropyridin-3-yl)-3,4- dihydroisoquinolin-1(2H)- one | 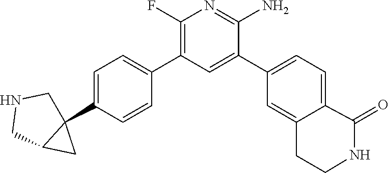 [SMILES: Nc1nc(F)c(-c2ccc([C@]34CNC[C@H]3C4)cc2)cc1-c1ccc2c(c1)CCNC2=O] |
| I-136 | (S)-6-(2-amino-6-chloro-5- (4-(2- isopropylmorpholino)phenyl) pyridin-3-yl)-3,4- dihydroisoquinolin-1(2H)- one | 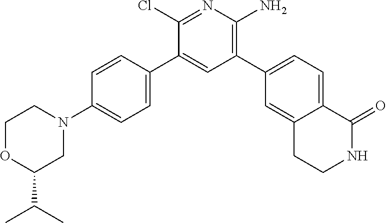 [SMILES: CC(C)[C@H]1CN(c2ccc(-c3cc(-c4ccc5c(c4)CCNC5=O)c(N)nc3Cl)cc2)CCO1] |
| I-137 | (R)-6-(2-amino-6-chloro-5- (2-fluoro-4-(2- isopropylmorpholino)phenyl) pyridin-3-yl)-3,4- dihydroisoquinolin-1(2H)- one | 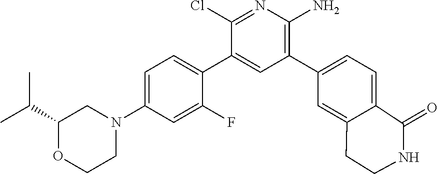 [SMILES: CC(C)[C@@H]1CN(c2ccc(-c3cc(-c4ccc5c(c4)CCNC5=O)c(N)nc3Cl)c(F)c2)CCO1] |
| I-138 | 6-(2-amino-6-fluoro-5-(4-(1- (2-hydroxyethyl)-1,2,3,6- tetrahydropyridin-4- yl)phenyl)pyridin-3-yl)-3,4- dihydroisoquinolin-1(2H)- one | 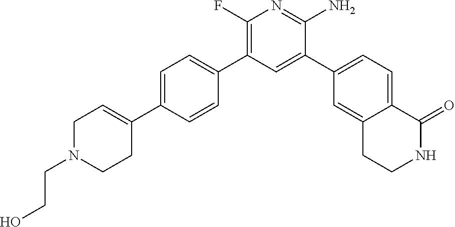 [SMILES: Nc1nc(F)c(-c2ccc(C3=CCN(CCO)CC3)cc2)cc1-c1ccc2c(c1)CCNC2=O] |
| I-139 | 6-(2-amino-6-fluoro-5-(4-(1- (2-methoxyethyl)-1,2,3,6- tetrahydropyridin-4- yl)phenyl)pyridin-3-yl)-3,4- dihydroisoquinolin-1(2H)- one | 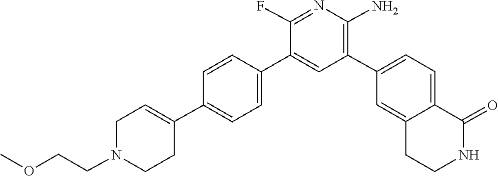 [SMILES: COCCN1CC=C(c2ccc(-c3cc(-c4ccc5c(c4)CCNC5=O)c(N)nc3F)cc2)CC1] |
| I-140 | 6-(2-amino-6-fluoro-5-(4- ((1R,5S)-3-(2-methoxyethyl)- 3-azabicyclo[3.1.0]hexan-1- yl)phenyl)pyridin-3-yl)-3,4- dihydroisoquinolin-1(2H)- one | 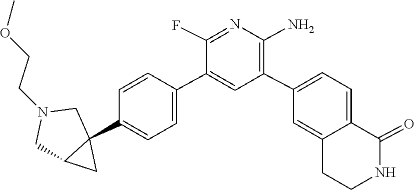 [SMILES: COCCN1C[C@H]2C[C@@]2(c2ccc(-c3cc(-c4ccc5c(c4)CCNC5=O)c(N)nc3F)cc2)C1] |
| I-141 | 6-(2-amino-6-fluoro-5-(4- ((1R,5S)-3-isopropyl-3- azabicyclo[3.1.0]heaxn-1- yl)phenyl)pyridin-3-yl)-7- fluoro-3,4- dihydroisoquinolin-1(2H)- one | 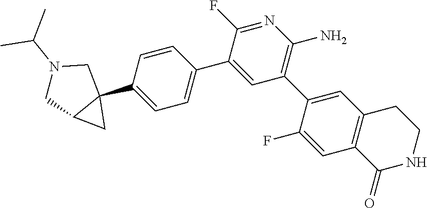 [SMILES: CC(C)N1C[C@H]2C[C@@]2(c2ccc(-c3cc(-c4cc5c(cc4F)C(=O)NCC5)c(N)nc3F)cc2)C1] |
| I-142 | 6-(2-amino-6-fluoro-5-(4- ((1S,5R)-3-isopropyl-3- azabicyclo[3.1.0]hexan-1- yl)phenyl)pyridin-3-yl)-7- fluoro-3,4- dihydroisoquinolin-1(2H)- one | 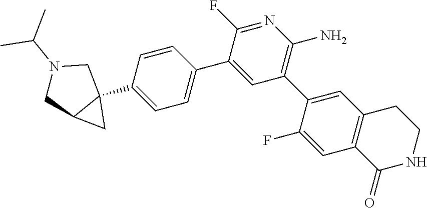 [SMILES: CC(C)N1C[C@@H]2C[C@]2(c2ccc(-c3cc(-c4cc5c(cc4F)C(=O)NCC5)c(N)nc3F)cc2)C1] |
| I-143 | 6-(2-amino-6-fluoro-5-(4- ((1R,5S)-3-isobutyryl-3- azabicyclo[3.1.0]hexan-1- yl)phenyl)pyridin-3-yl)-3,4- dihydroisoquinolin-1(2H)- one | 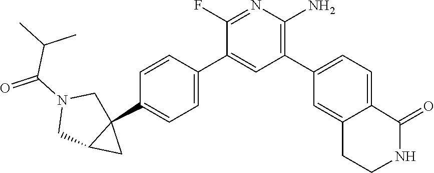 [SMILES: CC(C)C(=O)N1C[C@H]2C[C@@]2(c2ccc(-c3cc(-c4ccc5c(c4)CCNC5=O)c(N)nc3F)cc2)C1] |
| I-144 | 6-(2-amino-5-(4-(((2S,6S)- 2,6-dimethylpiperidin-4- yl)oxy)phenyl)-6- fluoropyridin-3-yl)-3,4- dihydroisoquinolin-1(2H)- one | 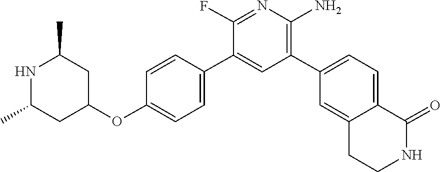 [SMILES: C[C@H]1CC(Oc2ccc(-c3cc(-c4ccc5c(c4)CCNC5=O)c(N)nc3F)cc2)C[C@H](C)N1] |
| I-145 | 6-(2-amino-6-fluoro-5-(7- (piperidin-4- yloxy)benzo[d][1,3]dioxol-4- yl)pyridin-3-yl)-3,4- dihydroisoquinolin-1(2H)- one | 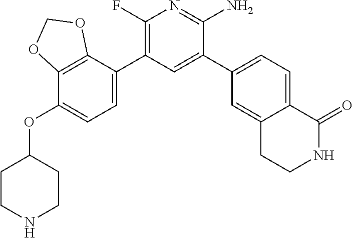 [SMILES: Nc1nc(F)c(-c2ccc(OC3CCNCC3)c3c2OCO3)cc1-c1ccc2c(c1)CCNC2=O] |
| I-146 | 6-(2-amino-5-(4-(azetidin-3- yloxy)phenyl)-6- fluoropyridin-3-yl)-3,4- dihydroisoquinolin-1(2H)- one | 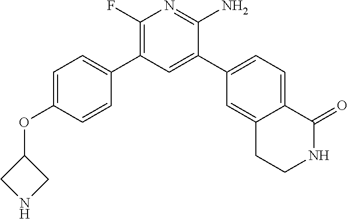 [SMILES: Nc1nc(F)c(-c2ccc(OC3CNC3)cc2)cc1-c1ccc2c(c1)CCNC2=O] |
| I-147 | 6-(2-amino-5-(4-((2,2- dimethylpiperidin-4- yl)oxy)phenyl)-6- fluoropyridin-3-yl)-3,4- dihydroisoquniolin-1(2H)- one | 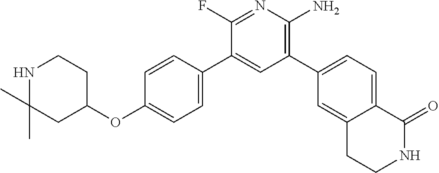 [SMILES: CC1(C)CC(Oc2ccc(-c3cc(-c4ccc5c(c4)CCNC5=O)c(N)nc3F)cc2)CCN1] |
| I-148 | 6-(2-amino-5-(2,3-difluoro-4- (piperidin-4-yloxy)phenyl)-6- fluoropyridin-3-yl)-3,4- dihydroisoquinolin-1(2H)- one | 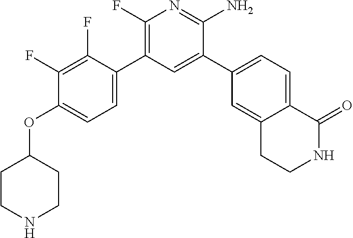 [SMILES: Nc1nc(F)c(-c2ccc(OC3CCNCC3)c(F)c2F)cc1-c1ccc2c(c1)CCNC2=O] |
| I-149 | 6-(2-amino--(2,5-difluoro-4- (piperidin-4-yloxy)phenyl)-6- fluoropyridin-3-yl)-3,4- dihydroisoquinolin-1(2H)- one | 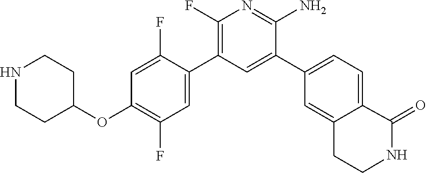 [SMILES: Nc1nc(F)c(-c2cc(F)c(OC3CCNCC3)cc2F)cc1-c1ccc2c(c1)CCNC2=O] |
| I-150 | 6-(2-amino-6-fluoro-5-(2- methoxy-4-(piperidin-4- yloxy)phenyl)pyridin-3-yl)- 3,4-dihydroisoquinolin- 1(2H)-one | 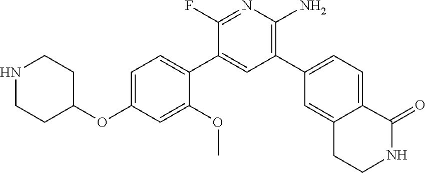 [SMILES: COc1cc(OC2CCNCC2)ccc1-c1cc(-c2ccc3c(c2)CCNC3=O)c(N)nc1F] |
| I-151 | 6-(2-amino-6-fluoro-5-(3- methoxy-4-(piperidin-4- yloxy)phenyl)pyridin-3-yl)- 3,4-dihydroisoquinolin- 1(2H)-one | 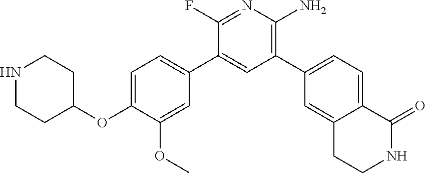 [SMILES: COc1cc(-c2cc(-c3ccc4c(c3)CCNC4=O)c(N)nc2F)ccc1OC1CCNCC1] |
| I-152 | 6-(2-amino-6-fluro-5-(4-(1- (oxetan-3-yl)piperidin-4- yl)phenyl)pyridin-3-yl)-3,4- dihydroisoquinolin-1(2H)- one | 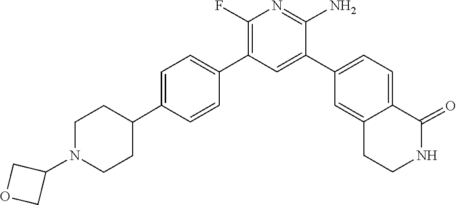 [SMILES: Nc1nc(F)c(-c2ccc(C3CCN(C4COC4)CC3)cc2)cc1-c1ccc2c(c1)CCNC2=O] |
| I-153 | 6-(2-amino-6-fluoro-5-(4-(1- (oxetan-3-yl)piperidin-2- yl)phenyl)pyridin-3-yl)-3,4- dihydroisoquinolin-1(2H)- one | 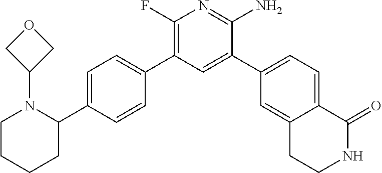 [SMILES: Nc1nc(F)c(-c2ccc(C3CCCCN3C3COC3)cc2)cc1-c1ccc2c(c1)CCNC2=O] |
| I-154 | 6-(2-amino-6-fluoro-5-(4-(4- (oxetan-3-yl)morpholin-3- yl)phenyl)pyridin-3-yl)-3,4- dihydroisoquinolin-1(2H)- one | 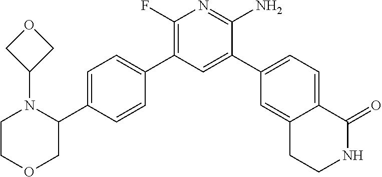 [SMILES: Nc1nc(F)c(-c2ccc(C3COCCN3C3COC3)cc2)cc1-c1ccc2c(c1)CCNC2=O] |
| I-155 | 6-(2-amino-6-fluoro-5-(4-(1- (oxetan-3-yl)-1,2,3,6- tetrahydropyridin-4- yl)phenyl)pyridin-3-yl)-3,4- dihydroisoquinolin-1(2H)- one | 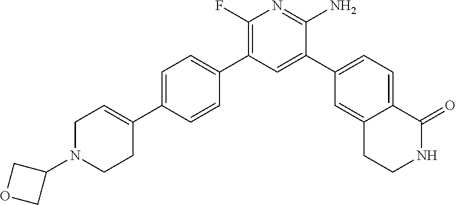 [SMILES: Nc1nc(F)c(-c2ccc(C3=CCN(C4COC4)CC3)cc2)cc1-c1ccc2c(c1)CCNC2=O] |
| I-156 | 6-(2-amino-6-fluoro-5-(4- ((1R,5S)-3-(oxetan-3-yl)-3- azabicyclo[3.1.0]hexan-1- yl)phenyl)pyridin-3-yl)-3,4- dihydroisoquinolin-1(2H)- one | 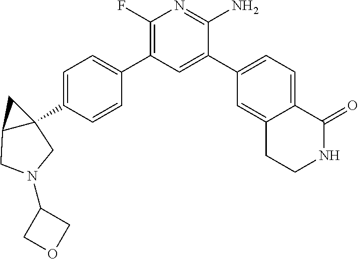 [SMILES: Nc1nc(F)c(-c2ccc([C@@]34C[C@@H]3CN(C3COC3)C4)cc2)cc1-c1ccc2c(c1)CCNC2=O] |
| I-157 | 6-(2-amino-5-(4-(3- (dimethylamino)pyrrolidin-1- yl)phenyl)-6-fluoropyridin-3- yl)-3,4-dihydroisoquinolin- 1(2H)-one | 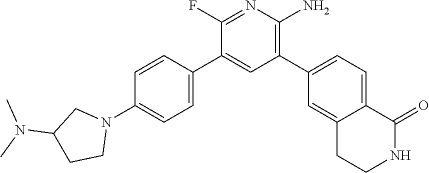 [SMILES: CN(C)C1CCN(c2ccc(-c3cc(-c4ccc5c(c4)CCNC5=O)c(N)nc3F)cc2)C1] |
| I-158 | 6-(2-amino-5-(4-(3- (ethyl(methyl)amino)pyrrolidin- 1-yl)phenyl)-6- fluoropyridin-3-yl)-3,4- dihydroisoquinolin-1(2H)- one | 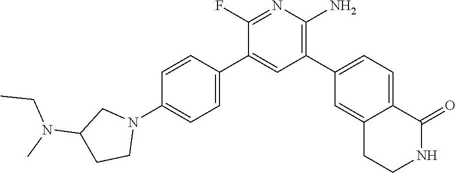 [SMILES: CCN(C)C1CCN(c2ccc(-c3cc(-c4ccc5c(c4)CCNC5=O)c(N)nc3F)cc2)C1] |
| I-159 | 6-(2-amino-6-fluoro-5-(4-(3- (methyl(2,2,2- trifluoroethyl)amino)pyrrolidin- 1-yl)phenyl)pyridin-3-yl)- 3,4-dihydroisoquinolin- 1(2H)-one | 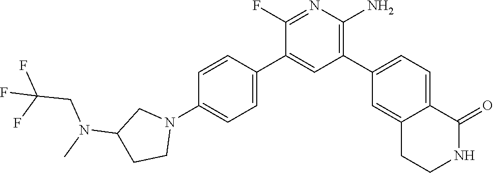 [SMILES: CN(CC(F)(F)F)C1CCN(c2ccc(-c3cc(-c4ccc5c(c4)CCNC5=O)c(N)nc3F)cc2)C1] |
| I-160 | (R)-6-(2-amino-6-fluoro-5-(4- (3-((2- methoxyethyl)(methyl)amino) pyrrolidin-1- yl)phenyl)pyridin-3-yl)-3,4- dihydroisoquinolin-1(2H)- one | 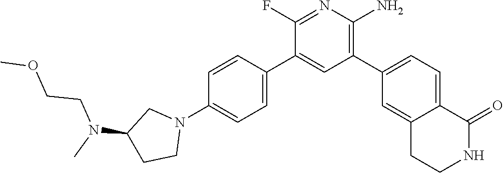 [SMILES: COCCN(C)[C@@H]1CCN(c2ccc(-c3cc(-c4ccc5c(c4)CCNC5=O)c(N)nc3F)cc2)C1] |
| I-161 | (R)-6-(2-amino-5-(4-(3- ((cyclopropylmethyl)(methyl) amino)pyrrolidin-1- yl)phenyl)-6-fluoropyridin-3- yl)-3,4-dihydroisoquinolin- 1(2H)-one | 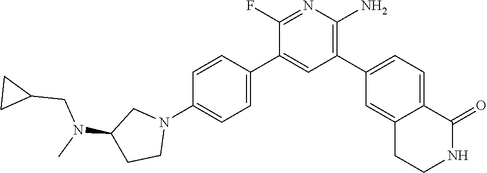 [SMILES: CN(CC1CC1)[C@@H]1CCN(c2ccc(-c3cc(-c4ccc5c(c4)CCNC5=O)c(N)nc3F)cc2)C1] |
| I-162 | 6-(2-amino-6-fluoro-5-(4-(3- (isobutyl(methyl)amino)pyrro- lidin-1-yl)phenyl)pyridin-3- yl)-3,4-dihydroisoquinolin- 1(2H)-one | 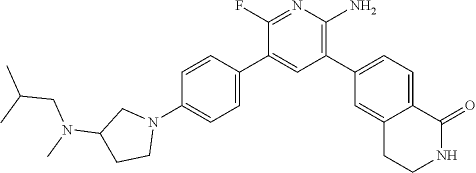 [SMILES: CC(C)CN(C)C1CCN(c2ccc(-c3cc(-c4ccc5c(c4)CCNC5=O)c(N)nc3F)cc2)C1] |
| I-163 | 6-(2-amnio-6-fluoro-5-(4-(4- fluoropiperidin-1- yl)phenyl)pyridin-3-yl)-3,4- dihydroisoquinolin-1(2H)- one | 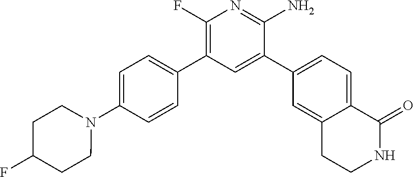 [SMILES: Nc1nc(F)c(-c2ccc(N3CCC(F)CC3)cc2)cc1-c1ccc2c(c1)CCNC2=O] |
| I-164 | 6-(2-amino-6-fluoro-5-(4-(1- isopropyl-3-methylpyrrolidin- 3-yl)phenyl)pyridin-3-yl)-3,4- dihydroisoquinolin-1(2H)- one | 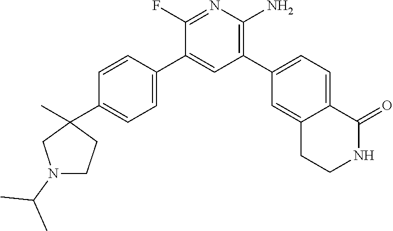 [SMILES: CC(C)N1CCC(C)(c2ccc(-c3cc(-c4ccc5c(c4)CCNC5=O)c(N)nc3F)cc2)C1] |
| I-165 | 6-(2-amino-6-fluoro-5-(4- ((1S,5R)-3-(2-methoxyethyl)- 3-azabicyclo[3.1.0]hexan-1- yl)phenyl)pyridin-3-yl)-3,4- dihydroisoquinolin-1(2H)- one | 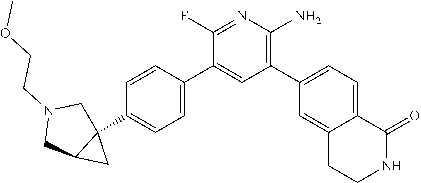 [SMILES: COCCN1C[C@@H]2C[C@]2(c2ccc(-c3cc(-c4ccc5c(c4)CCNC5=O)c(N)nc3F)cc2)C1] |
| I-166 | 6-(2-amino-6-chloro-5-(4-(1- (2-methoxyethyl)piperidin-4- yl)phenyl)pyridin-3-yl)-3,4- dihydroisoquinolin-1(2H)- one | 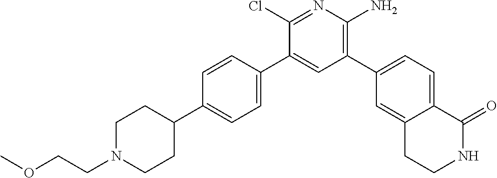 [SMILES: COCCN1CCC(c2ccc(-c3cc(-c4ccc5c(c4)CCNC5=O)c(N)nc3Cl)cc2)CC1] |
| I-167 | 6-(2-amino-6-fluoro-5-(4-(4- hydroxy-1-(2- methoxyethyl)piperidin-4- yl)phenyl)pyridin-3-yl)-3,4- dihydroisoquinolin-1(2H)- one | 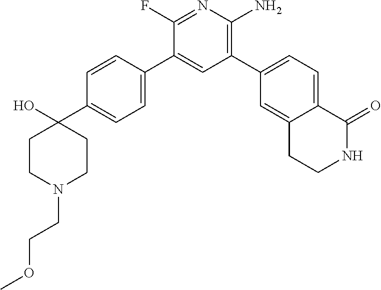 [SMILES: COCCN1CCC(O)(c2ccc(-c3cc(-c4ccc5c(c4)CCNC5=O)c(N)nc3F)cc2)CC1] |
| I-168 | 6-(2-amino-6-chloro-5-(4-(4- (2,2-difluoroethyl)piperazin- 1-yl)phenyl)pyridin-3-yl)-3,4- dihydroisoquinolin-1(2H)- one | 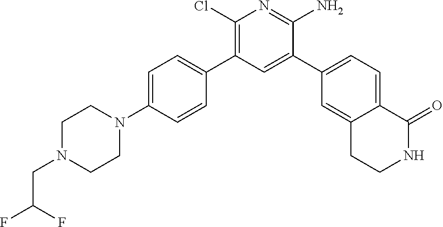 [SMILES: Nc1nc(Cl)c(-c2ccc(N3CCN(CC(F)F)CC3)cc2)cc1-c1ccc2c(c1)CCNC2=O] |
| I-169 | 6-(2-amino-6-chloro-5-(4-(4- isopropylpiperazin-1- yl)phenyl)pyridin-3-yl)-3,4- dihydroisoquinolin-1(2H)- one | 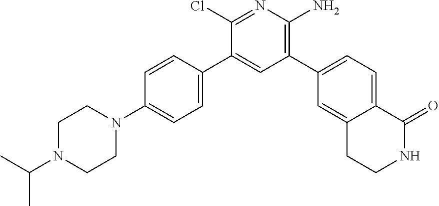 [SMILES: CC(C)N1CCN(c2ccc(-c3cc(-c4ccc5c(c4)CCNC5=O)c(N)nc3Cl)cc2)CC1] |
| I-170 | 6-(6′-amino-2′-fluoro-5-(4- isoporpylpiperazin-1-yl)- [2,3′-bipyridin]-5′-yl)-7-fluoro- 3,4-dihydroisoquinolin- 1(2H)-one | 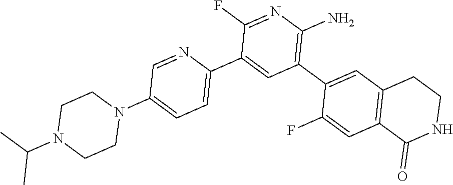 [SMILES: CC(C)N1CCN(c2ccc(-c3cc(-c4cc5c(cc4F)C(=O)NCC5)c(N)nc3F)nc2)CC1] |
| I-171 | 6-(2-amino-5-(4-(6,6- difluoro-3-isopropyl-3- azabicyclo[3.1.0]hexan-1- yl)phenyl)-6-fluoropyridin-3- yl)-3,4-dihydroisoquinolin- 1(2H)-one | 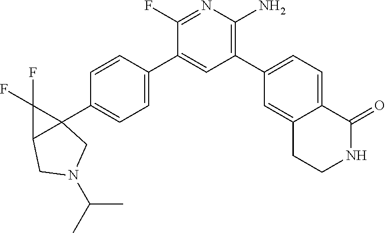 [SMILES: CC(C)N1CC2C(F)(F)C2(c2ccc(-c3cc(-c4ccc5c(c4)CCNC5=O)c(N)nc3F)cc2)C1] |
| I-172 | 6-(2-amino-6-fluoro-5-(4- (piperazin-1- yl)phenyl)pyridin-3-yl)-7- fluoro-3,4- dihydroisoquinolin-1(2H)- one 2,2,2-trifluoroacetate | 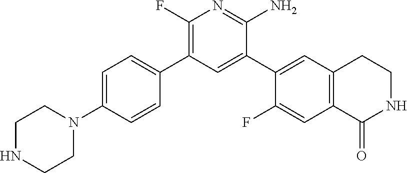 [SMILES: Nc1nc(F)c(-c2ccc(N3CCNCC3)cc2)cc1-c1cc2c(cc1F)C(=O)NCC2] |
| I-173 | tert-butyl 6-(6-amino-2- fluoro-5-(1-oxo-1,2,3,4- tetrahydroisoquinolin-6- yl)pyridin-3-yl)-4H- spiro[benzo[d][1,3]dioxine- 2,4′-piperidine]-1′- carboxylate | 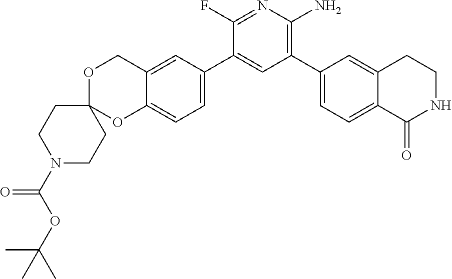 [SMILES: CC(C)(C)OC(=O)N1CCC2(CC1)OCc1cc(-c3cc(-c4ccc5c(c4)CCNC5=O)c(N)nc3F)ccc1O2] |
| I-174 | tert-butyl 6-(6-amino-2- fluoro-5-(1-oxo-1,2,3,4- tetrahydroisoquinolin-6- yl)pyridin-3-yl)-4- oxospiro[chromane-2,4′- piperidine]-1′-carboxylate | 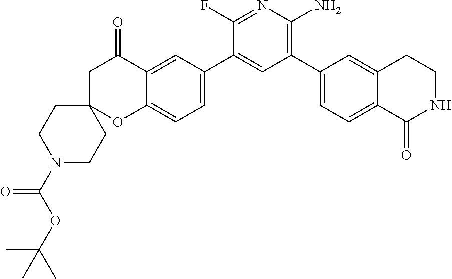 [SMILES: CC(C)(C)OC(=O)N1CCC2(CC1)CC(=O)c1cc(-c3cc(-c4ccc5c(c4)CCNC5=O)c(N)nc3F)ccc1O2] |
| I-175 | tert-butyl 6-(6-amino-2- fluoro-5-(1-oxo-1,2,3,4- tetrahydroisoquinolin-6- yl)pyridin-3- yl)spiro[chromane-2,4′- piperidine]-1′-carboxylate | 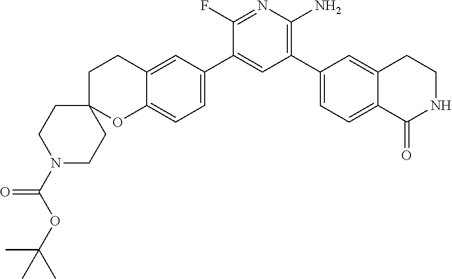 [SMILES: CC(C)(C)OC(=O)N1CCC2(CCc3cc(-c4cc(-c5ccc6c(c5)CCNC6=O)c(N)nc4F)ccc3O2)CC1] |
| I-176 | tert-butyl 4-(4-(6-amino-2- fluoro-5-(1-oxo-1,2,3,4- tetrahydroisoquinolin-6- yl)pyridin-3-yl)-2- cyanophenoxy)piperidine-1- carboxylate | 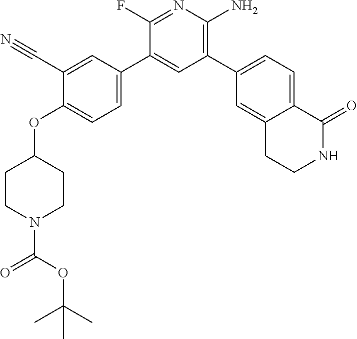 [SMILES: CC(C)(C)OC(=O)N1CCC(Oc2ccc(-c3cc(-c4ccc5c(c4)CCNC5=O)c(N)nc3F)cc2C#N)CC1] |
| I-177 | tert-butyl 4-(4-(6-amino-2- fluoro-5-(1-oxo-1,2,3,4- tetrahydroisoquinolin-6- yl)pyridin-3-yl)-2- (trifluoromethyl)phenyl)piper- azine-1-carboxylate | 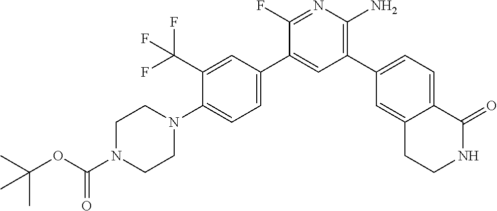 [SMILES: CC(C)(C)OC(=O)N1CCN(c2ccc(-c3cc(-c4ccc5c(c4)CCNC5=O)c(N)nc3F)cc2C(F)(F)F)CC1] |
| I-178 | 6-(2-amino--(4- (((2S,4s,6R)-2,6- dimethylpiperidin-4- yl)oxy)phenyl)-6- fluoropyridin-3-yl)-3,4- dihydroisoquinolin-1(2H)- one | 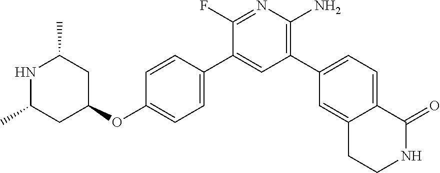 [SMILES: C[C@@H]1C[C@@H](Oc2ccc(-c3cc(-c4ccc5c(c4)CCNC5=O)c(N)nc3F)cc2)C[C@H](C)N1] |
| I-179 | 6-(2-amio-6-fluoro-5-(3- fluoro-4-(piperidin-4- yloxy)phenyl)pyridin-3-yl)- 3,4-dihydroisoquinolin- 1(2H)-one | 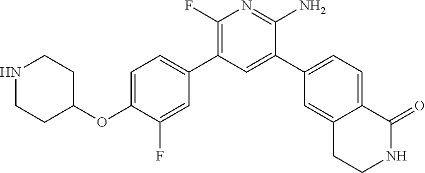 [SMILES: Nc1nc(F)c(-c2ccc(OC3CCNCC3)c(F)c2)cc1-c1ccc2c(c1)CCNC2=O] |
| I-180 | (R)-6-(2-amino-6-fluoro-5-(4- (piperidin-3- yloxy)phenyl)pyridin-3-yl)-7- fluoro-3,4- dihydroisoquinolin-1(2H)- one | 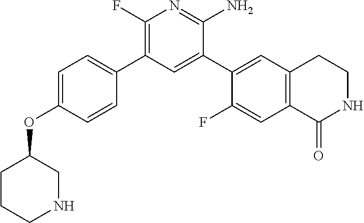 [SMILES: Nc1nc(F)c(-c2ccc(O[C@@H]3CCCNC3)cc2)cc1-c1cc2c(cc1F)C(=O)NCC2] |
| I-181 | 6-(2-amio-6-fluoro-5-(4H- spiro[benzo[d][1,3]dioxine- 2,4′-piperidin]-6-yl)pyridin-3- yl)-3,4-dihydroisoquinolin- 1(2H)-one | 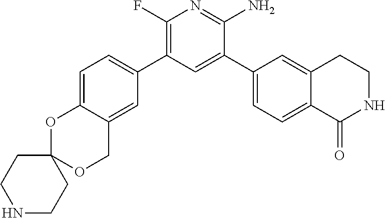 [SMILES: Nc1nc(F)c(-c2ccc3c(c2)COC2(CCNCC2)O3)cc1-c1ccc2c(c1)CCNC2=O] |
| I-182 | 6-(6-amino-2-fluoro-5-(1- oxo-1,2,3,4- tetrahydroisoquinolin-6- yl)pyridin-3- yl)spiro[chromane-2,4′- piperidin]-4-one 2,2,2- trifluoroacetate | 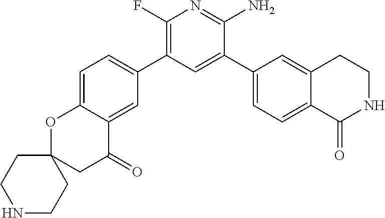 [SMILES: Nc1nc(F)c(-c2ccc3c(c2)C(=O)CC2(CCNCC2)O3)cc1-c1ccc2c(c1)CCNC2=O] |
| I-183 | 6-(2-amino-6-fluoro-5- (spiro[chromane-2,4′- piperidin]-6-yl)pyridin-3-yl)- 3,4-dihydroisoquinolin- 1(2H)-one | 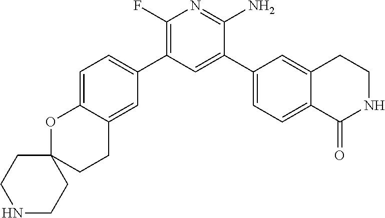 [SMILES: Nc1nc(F)c(-c2ccc3c(c2)CCC2(CCNCC2)O3)cc1-c1ccc2c(c1)CCNC2=O] |
| I-185 | 5-(6-amino-2-fluoro-5-(1- oxo-1,2,3,4- tetrahydroisoquinolin-6- yl)pyridin-3-yl)-2-(piperidin- 4-yloxy)benzonitrile | 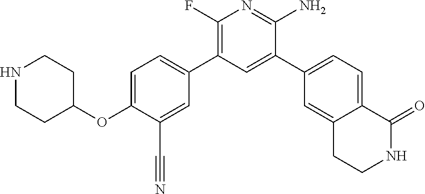 [SMILES: N#Cc1cc(-c2cc(-c3ccc4c(c3)CCNC4=O)c(N)nc2F)ccc1OC1CCNCC1] |
| I-185 | 6-(2-amino-6-fluoro-5-(4- (piperazin-1-yl)-3- (trifluoromethyl)phenyl)pyridin- 3-yl)-3,4- dihydroisoquinolin-1(2H)- one | 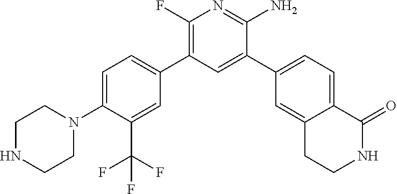 [SMILES: Nc1nc(F)c(-c2ccc(N3CCNCC3)c(C(F)(F)F)c2)cc1-c1ccc2c(c1)CCNC2=O] |
| I-186 | 6-(2-amino-6-fluoro-5-(4-((1- (2,2,2-trifluororethyl)piperidin- 4-yl)oxy)phenyl)pyridin-3-l)- 3,4-dihydroisoquinolin- 1(2H)-one | 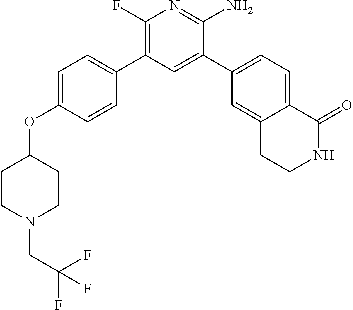 [SMILES: Nc1nc(F)c(-c2ccc(OC3CCN(CC(F)(F)F)CC3)cc2)cc1-c1ccc2c(c1)CCNC2=O] |
| I-187 | 6-(2-amino-6-fluoro-5-(4-((1- (3,3,3- trifluoropropyl)piperidin-4- yl)oxy)phenyl)pyridin-3-yl)- 3,4-dihydroisoquinolin- 1(2H)-one | 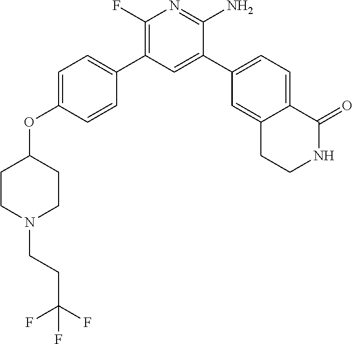 [SMILES: Nc1nc(F)c(-c2ccc(OC3CCN(CCC(F)(F)F)CC3)cc2)cc1-c1ccc2c(c1)CCNC2=O] |
| I-188 | 6-(2-amino-6-fluoro-5-(4-((1- (4,4,4-trifluorobutyl)piperidin- 4-yl)oxy)phenyl)pyridin-3-yl)- 3,4-dihydroisoquinolin- 1(2H)-one | 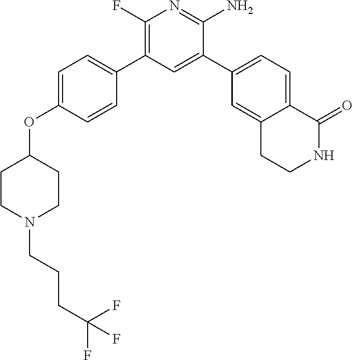 [SMILES: Nc1nc(F)c(-c2ccc(OC3CCN(CCCC(F)(F)F)CC3)cc2)cc1-c1ccc2c(c1)CCNC2=O] |
| I-189 | 6-(2-amino-6-fluoro-5-(4-((1- (2-methoxyethyl)piperidin-4- yl)oxy)phenyl)pyridin-3-yl)- 3,4-dihydroisoquinolin- 1(2H)-one | 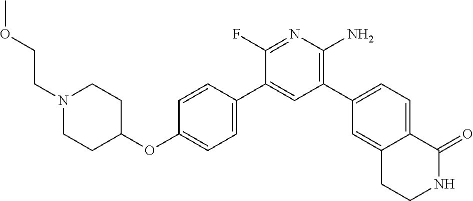 [SMILES: COCCN1CCC(Oc2ccc(-c3cc(-c4ccc5c(c4)CCNC5=O)c(N)nc3F)cc2)CC1] |
| I-190 | 6-(2-amino-6-fluoro-5-(4- (piperidin-4-yloxy)-3- (trifluoromethyl)phenyl)pyridin- 3-yl)-3,4- dihydroisoquinolin-1(2H)- one | 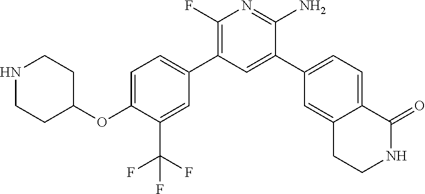 [SMILES: Nc1nc(F)c(-c2ccc(OC3CCNCC3)c(C(F)(F)F)c2)cc1-c1ccc2c(c1)CCNC2=O] |
| I-191 | 6-(5-(1′-acetyl-4H- spiro[benzo[d][1,3]dioxine- 2,4′-piperidin]-6-yl)-2-amino- 6-fluoropyridin-3-yl)-3,4- dihydroisoquinolin-1(2H)- one | 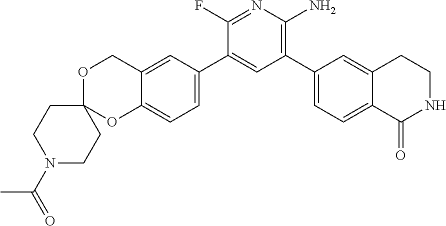 [SMILES: CC(=O)N1CCC2(CC1)OCc1cc(-c3cc(-c4ccc5c(c4)CCNC5=O)c(N)nc3F)ccc1O2] |
| I-192 | 1′-acetyl-6-(6-amino-2- fluoro-5-(1-oxo-1,2,3,4- tetrahydroisoquinolin-6- yl)pyridin-3- yl)spiro[chromane-2,4′- piperidin]-4-one | 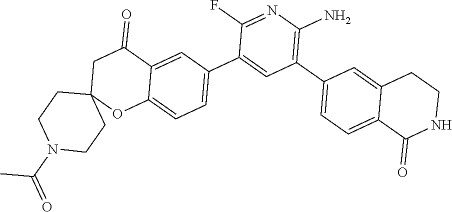 [SMILES: CC(=O)N1CCC2(CC1)CC(=O)c1cc(-c3cc(-c4ccc5c(c4)CCNC5=O)c(N)nc3F)ccc1O2] |
| I-193 | 6-(5-(1′- acetylspiro[chromane-2,4′- piperidin]-6-yl)-2-amino-6- fluoropyridin-3-yl)-3,4- dihydroisoquinolin-1(2H)- one | 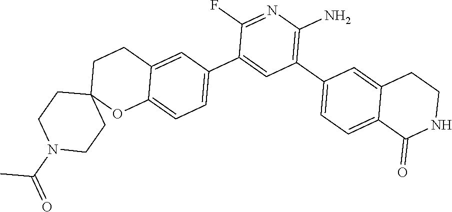 [SMILES: CC(=O)N1CCC2(CCc3cc(-c4cc(-c5ccc6c(c5)CCNC6=O)c(N)nc4F)ccc3O2)CC1] |
| I-194 | 2-((1-acetylpiperidin-4- yl)oxy)-5-(6-amino-2-fluoro- 5-(1-oxo-1,2,3,4- tetrahydroisoquinolin-6- yl)pyridin-3-yl)benzonitrile | 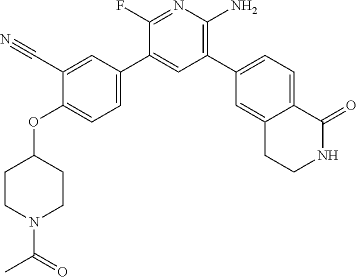 [SMILES: CC(=O)N1CCC(Oc2ccc(-c3cc(-c4ccc5c(c4)CCNC5=O)c(N)nc3F)cc2C#N)CC1] |
| I-195 | 6-(2-amino-6-fluoro-5-(1′- (2,2,2-trifluoroethyl)-4H- spiro[benzo[d][1,3]dioxine- 2,4′-piperidin]-6-yl)pyridin-3- yl)-3,4-dihydroisoquinolin- 1(2H)-one | 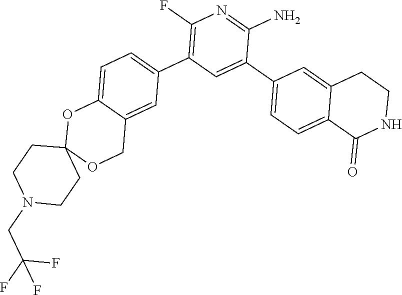 [SMILES: Nc1nc(F)c(-c2ccc3c(c2)COC2(CCN(CC(F)(F)F)CC2)O3)cc1-c1ccc2c(c1)CCNC2=O] |
| I-196 | 6-(6-amino-2-fluoro-5-(1- oxo-1,2,3,4- tetrahydroisoquinolin-6- yl)pyridin-3-yl)-1′-(2,2,2- trifluoroethyl)spiro[chromane- 2,4′-piperidin]-4-one | 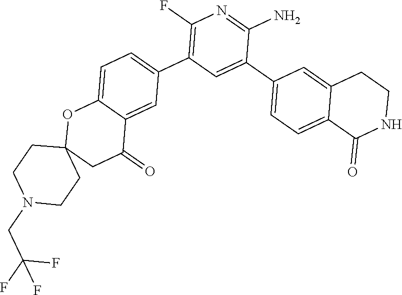 [SMILES: Nc1nc(F)c(-c2ccc3c(c2)C(=O)CC2(CCN(CC(F)(F)F)CC2)O3)cc1-c1ccc2c(c1)CCNC2=O] |
| I-197 | 6-(2-amino-6-fluoro-5-(1′- (2,2,2- trifluoroethyl)spiro[chromane- 2,4′-piperidin]-6-yl)pyridin-3- y)-3,4-dihydroisoqouinolin- 1(2H)-one | 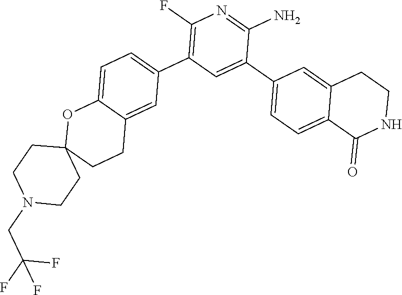 [SMILES: Nc1nc(F)c(-c2ccc3c(c2)CCC2(CCN(CC(F)(F)F)CC2)O3)cc1-c1ccc2c(c1)CCNC2=O] |
| I-198 | 5-(6-amino-2-fluoro-5-(1- oxo-1,2,3,4- tetrahydroisoquinolin-6- yl)pyridin-3-yl)-2-((1-(2,2,2- trifluoroethyl)piperidin-4- yl)oxy)benzonitrile | 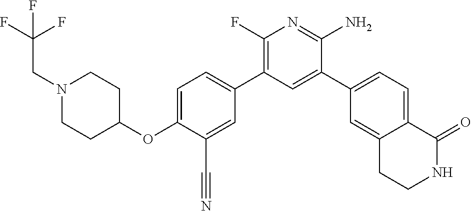 [SMILES: N#Cc1cc(-c2cc(-c3ccc4c(c3)CCNC4=O)c(N)nc2F)ccc1OC1CCN(CC(F)(F)F)CC1] |
| I-199 | 6-(5-(4-((1-acetylpiperidin-4- yl)oxy)-3- (trifluoromethyl)phenyl)-2- amino-6-fluoropyridin-3-yl)- 3,4-dihydroisoquinolin- 1(2H)-one | 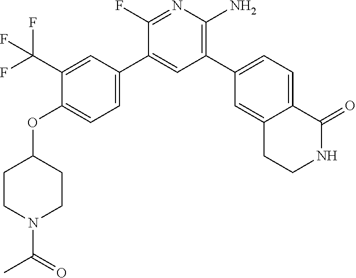 [SMILES: CC(=O)N1CCC(Oc2ccc(-c3cc(-c4ccc5c(c4)CCNC5=O)c(N)nc3F)cc2C(F)(F)F)CC1] |
| I-200 | 6-(5-(4-(4-acetylpiperazin-1- yl)-3- (trifluoromethyl)phenyl)-2- amino-6-fluoropyridin-3-yl)- 3,4-dihydroisoquinolin- 1(2H)-one | 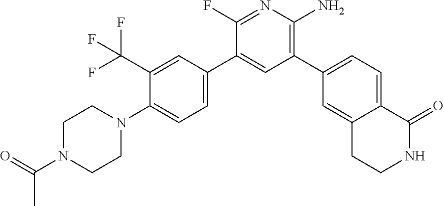 [SMILES: CC(=O)N1CCN(c2ccc(-c3cc(-c4ccc5c(c4)CCNC5=O)c(N)nc3F)cc2C(F)(F)F)CC1] |
| I-201 | 6-(2-amino-6-fluoro-5-(4-((1- (2,2,2-trifluoroethyl)piperidin- 4-yl)oxy)-3- (trifluoromethyl)phenyl)pyridin- 3-yl)-3,4- dihydroisoquinolin-1(2H)- one | 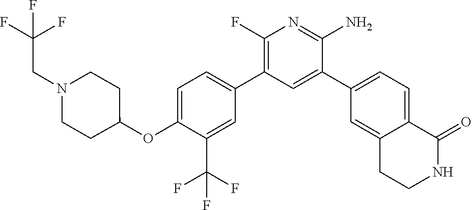 [SMILES: Nc1nc(F)c(-c2ccc(OC3CCN(CC(F)(F)F)CC3)c(C(F)(F)F)c2)cc1-c1ccc2c(c1)CCNC2=O] |
| I-202 | 6-(2-amino-6-fluoro-5-(4-(4- (2,2,2- trifluoroethyl)piperazin-1-yl)- 3- (trifluoromethyl)phenyl)pyridin- 3-yl)-3,4- dihydroisoquinolin-1(2H)- one | 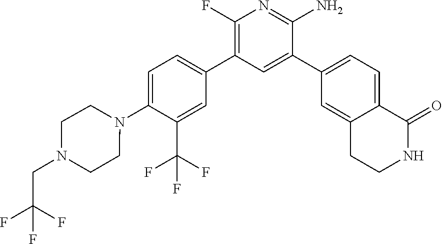 [SMILES: Nc1nc(F)c(-c2ccc(N3CCN(CC(F)(F)F)CC3)c(C(F)(F)F)c2)cc1-c1ccc2c(c1)CCNC2=O] |
| I-203 | 6-(2-amino-5-(4-((1- (cyclopropanecarbonyl)piper- idin-4-yl)oxy)phenyl)-6- fluoropyridin-3-yl)-3,4- dihydroisoquinolin-1(2H)- one | 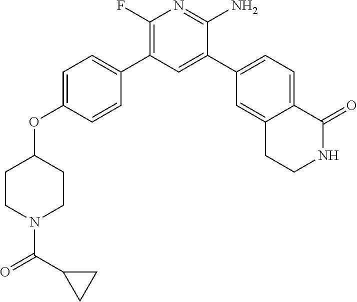 [SMILES: Nc1nc(F)c(-c2ccc(OC3CCN(C(=O)C4CC4)CC3)cc2)cc1-c1ccc2c(c1)CCNC2=O] |
| I-204 | 6-(2-amino-5-(3,4-dihydro- 2H-benzo[b][1,4]dioxepin-7- yl)-6-fluoropyridin-3-yl)-3,4- dihydroisoquinolin-1(2H)- one | 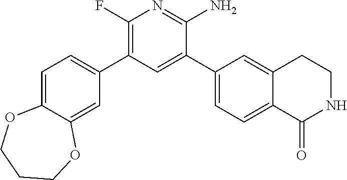 [SMILES: Nc1nc(F)c(-c2ccc3c(c2)OCCCO3)cc1-c1ccc2c(c1)CCNC2=O] |
| I-205 | 6-(2-amino-5-(3,4-dihydro- 2H-benzo[b][1,4]dioxepin-7- yl)-6-fluoropyridin-3-yl)-7- fluoro-3,4- dihydroisoquinolin-1(2H)- one | 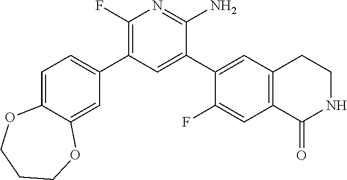 [SMILES: Nc1nc(F)c(-c2ccc3c(c2)OCCCO3)cc1-c1cc2c(cc1F)C(=O)NCC2] |
| I-206 | 6-(2-amino-6-fluoro-5-(4-((4- methyl-1,4-diazepan-1- yl)sulfonyl)pehnyl)pyridin-3- yl)-3,4-dihydroisoquinolin- 1(2H)-one | 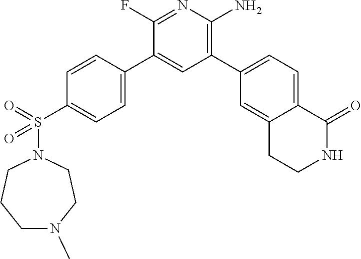 [SMILES: CN1CCCN(S(=O)(=O)c2ccc(-c3cc(-c4ccc5c(c4)CCNC5=O)c(N)nc3F)cc2)CC1] |
| I-207 | 6-(2-amino-6-fluoro-5-(4-((4- methylpiperazin-1- yl)sulfonyl)phenyl)pyridin-3- yl)-3,4-dihydroisoquinolin- 1(2H)-one | 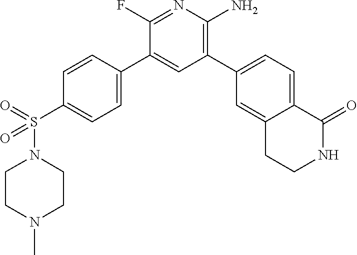 [SMILES: CN1CCN(S(=O)(=O)c2ccc(-c3cc(-c4ccc5c(c4)CCNC5=O)c(N)nc3F)cc2)CC1] |
| I-208 | 6-(2-amino-6-fluoro-5-(4-(4- methoxy-1-(2- methoxyethyl)piperidin-4- yl)phenyl)pyridin-3-yl)-3,4- dihydroisoquinolin-1(2H)- one | 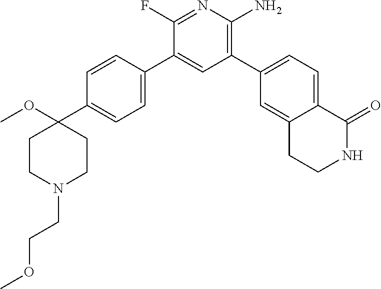 [SMILES: COCCN1CCC(OC)(c2ccc(-c3cc(-c4ccc5c(c4)CCNC5=O)c(N)nc3F)cc2)CC1] |
| I-209 | 6-(2-amino-6-chloro-5-(4-(4- isopropylpiperazin-1- yl)phenyl)pyridin-3-yl)-7- fluoro-3,4- dihydroisoquinolin-1(2H)- one | 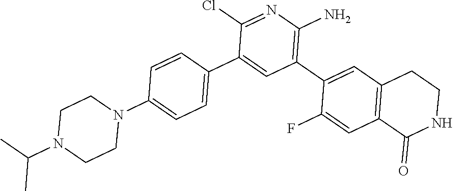 [SMILES: CC(C)N1CCN(c2ccc(-c3cc(-c4cc5c(cc4F)C(=O)NCC5)c(N)nc3Cl)cc2)CC1] |
| I-210 | 6-(2-amino-5-(4-(4- (cyclopropylmethyl)piperazin- 1-yl)phenyl)-6-fluoropyridin- 3-yl)-3,4-dihydroisoquinolin- 1(2H)-one | 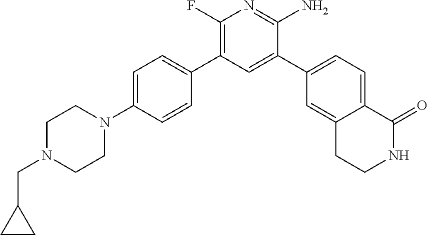 [SMILES: Nc1nc(F)c(-c2ccc(N3CCN(CC4CC4)CC3)cc2)cc1-c1ccc2c(c1)CCNC2=O] |
| I-211 | 6-(2-amino-5-(4-(4- ethylpiperazin-1-yl)phenyl)- 6-fluoropyridin-3-yl)-7-fluoro- 3,4-dihydroisoquinolin- 1(2H)-one | 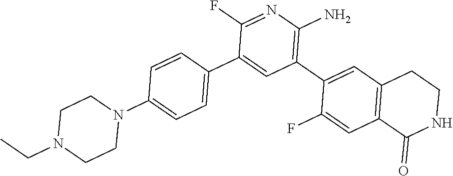 [SMILES: CCN1CCN(c2ccc(-c3cc(-c4cc5c(cc4F)C(=O)NCC5)c(N)nc3F)cc2)CC1] |
| I-212 | 6-(2-amino-6-fluoro-5-(4-(4- (2-methoxyethyl)piperazin-1- yl)phenyl)pyridin-3-yl)-7- fluoro-3,4- dihydroisoquinolin-1(2H)- one | 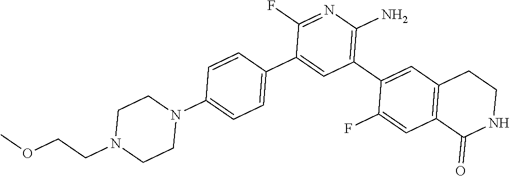 [SMILES: COCCN1CCN(c2ccc(-c3cc(-c4cc5c(cc4F)C(=O)NCC5)c(N)nc3F)cc2)CC1] |
| I-213 | 6-(2-amino-5-(4-(4- (cyclopropylmethyl)piperaizn- 1-yl)phenyl)-6-fluoropyridin- 3-yl)-7-fluoro-3,4- dihydroisoquinolin-1(2H)- one | 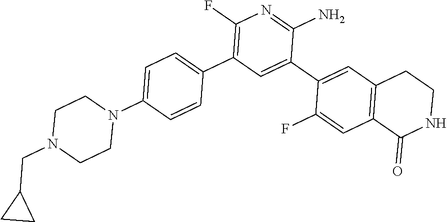 [SMILES: Nc1nc(F)c(-c2ccc(N3CCN(CC4CC4)CC3)cc2)cc1-c1cc2c(cc1F)C(=O)NCC2] |
| I-214 | 6-(2-amino-6-fluoro-5-(1′-(2- methoxyethyl)-4H- spiro[benzo[d][1,3]dioxine- 2,4′-piperidin]-6-yl)pyridin-3- yl)-3,4-dihydroisoquinolin- 1(2H)-one | 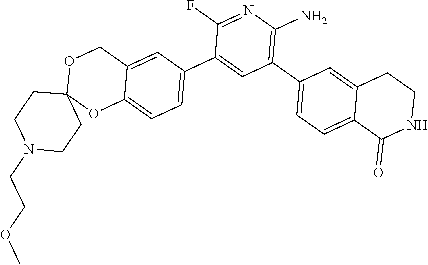 [SMILES: COCCN1CCC2(CC1)OCc1cc(-c3cc(-c4ccc5c(c4)CCNC5=O)c(N)nc3F)ccc1O2] |
| I-215 | 6-(6-amino-2-fluoro-5-(1- oxo-1,2,3,4- tetrahydroisoquinolin-6- yl)pyridin-3-yl)-1′-(2- methoxyethyl)spiro[chromane- 2,4′-piperidin]-4-one | 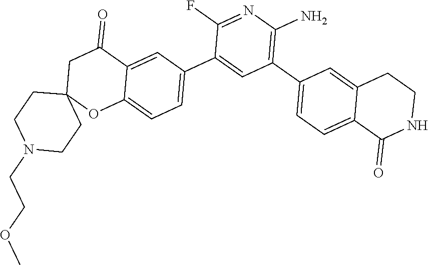 [SMILES: COCCN1CCC2(CC1)CC(=O)c1cc(-c3cc(-c4ccc5c(c4)CCNC5=O)c(N)nc3F)ccc1O2] |
| I-216 | 6-(2-amino-6-fluoro-5-(1′-(2- methoxyethyl)spiro[chromane- 2,4′-piperidin]-6-yl)pyridin- 3-yl)-3,4-dihydroisoquinolin- 1(2H)-one | 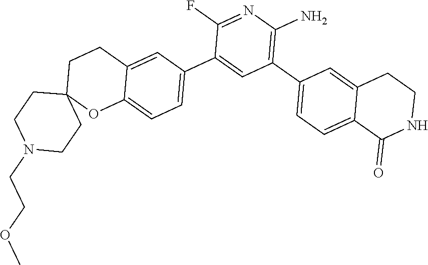 [SMILES: COCCN1CCC2(CCc3cc(-c4cc(-c5ccc6c(c5)CCNC6=O)c(N)nc4F)ccc3O2)CC1] |
| I-217 | 5-(6-amino-2-fluoro-5-(1- oxo-1,2,3,4- tetrahydroisoquinolin-6- yl)pyridin-3-yl)-2-((1-(2- methoxyethyl)piperidin-4- yl)oxy)benzonnitrile | 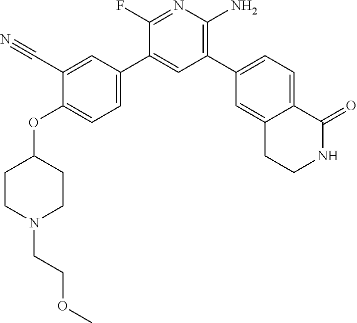 [SMILES: COCCN1CCC(Oc2ccc(-c3cc(-c4ccc5c(c4)CCNC5=O)c(N)nc3F)cc2C#N)CC1] |
| I-218 | 6-(2-amino-6-fluoro-5-(1′- isopropyl-4H- spiro[benzo[d][1,3]dioxine- 2,4′-piperidin]-6-yl)pyridin-3- yL)-3,4-dihydroisoquinolin- 1(2H)-one/ | 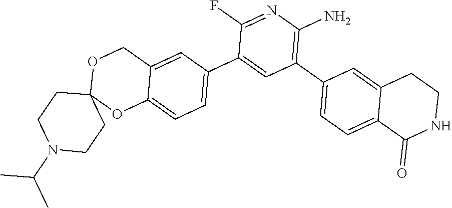 [SMILES: CC(C)N1CCC2(CC1)OCc1cc(-c3cc(-c4ccc5c(c4)CCNC5=O)c(N)nc3F)ccc1O2] |
| I-219 | 6-(2-amino-6-fluoro-5-(4- (piperazin-1-yl)-3- (trifluoromethyl)phenyl)pyridin- 3-yl)-7-fluoro-3,4- dihydroisoquinolin-1(2H)- one | 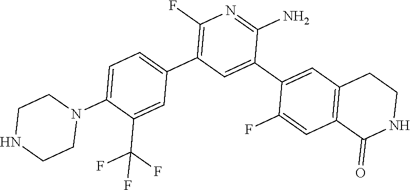 [SMILES: Nc1nc(F)c(-c2ccc(N3CCNCC3)c(C(F)(F)F)c2)cc1-c1cc2c(cc1F)C(=O)NCC2] |
| I-220 | 6-(2-amino-6-fluoro-5-(4- ((1R,5S)-3-(2- methoxyacetyl)-3- azabicyclo[3.1.0]hexan-1- yl)phenyl)pyridin-3-yl)-3,4- dihydroisoquinolin-1(2H)- one | 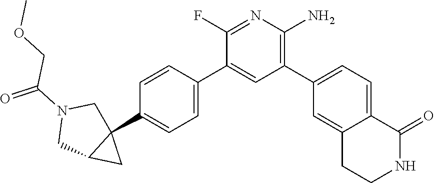 [SMILES: COCC(=O)N1C[C@H]2C[C@@]2(c2ccc(-c3cc(-c4ccc5c(c4)CCNC5=O)c(N)nc3F)cc2)C1] |
| I-221 | 6-(6′-(4-acetylpiperazin-1- yl)-6-amino-2-fluoro-[3,3′- bipyridin]-5-yl)-7-fluoro-3,4- dihydroisoquinolin-1(2H)- one 2,2,2-trifluoroacetate | 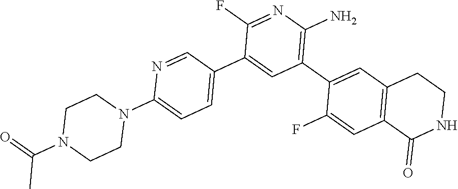 [SMILES: CC(=O)N1CCN(c2ccc(-c3cc(-c4cc5c(cc4F)C(=O)NCC5)c(N)nc3F)cn2)CC1] |
| I-222 | 6-(6-amino-2-fluoro-5-(1- oxo-1,2,3,4- tetrahydroisoquinolin-6- yl)pyridin-3-yl)-1′- isopropylspiro[chromane- 2,4′-piperidin]-4-one | 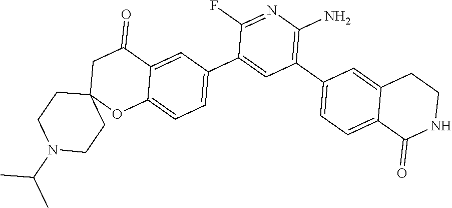 [SMILES: CC(C)N1CCC2(CC1)CC(=O)c1cc(-c3cc(-c4ccc5c(c4)CCNC5=O)c(N)nc3F)ccc1O2] |
| I-223 | 6-(2-amino-6-fluoro-5-(1′- isopropylspiro[chromane- 2,4′-piperidin]-6-yl)pyridin-3- yl)-3,4-dihydroisoquinolin- 1(2H)-one | 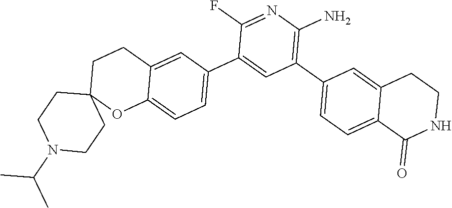 [SMILES: CC(C)N1CCC2(CCc3cc(-c4cc(-c5ccc6c(c5)CCNC6=O)c(N)nc4F)ccc3O2)CC1] |
| I-224 | 5-(6-amino-2-fluoro-5-(1- oxo-1,2,3,4- tetrahydroisoquinolin-6- yl)pyridin-3-yl)-2-((1- isopropylpiperidin-4- yl)oxy)benzonitrile | 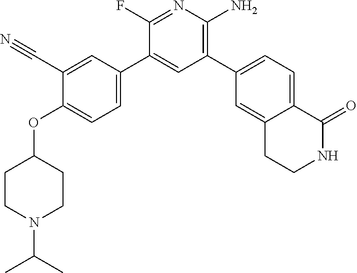 [SMILES: CC(C)N1CCC(Oc2ccc(-c3cc(-c4ccc5c(c4)CCNC5=O)c(N)nc3F)cc2C#N)CC1] |
| I-225 | 6-(2-amino-5-(4-((1R,5S)-3- (dimethylglycyl)-3- azabicyclo[3.1.0]hexan-1- yl)phenyl)-6-fluoropyridin-3- yl)-3,4-dihydroisoquinolin- 1(2H)-one | 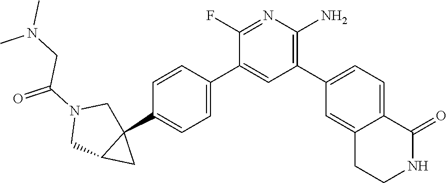 [SMILES: CN(C)CC(=O)N1C[C@H]2C[C@@]2(c2ccc(-c3cc(-c4ccc5c(c4)CCNC5=O)c(N)nc3F)cc2)C1] |
| I-226 | 6-(6-amino-2-fluoro-6′-(4- isopropylpiperazin-1-yl)- [3,3′-bipyriidn]-5-yl)-7-fluoro- 3,4-dihydroisoquinolin- 1(2H)-one | 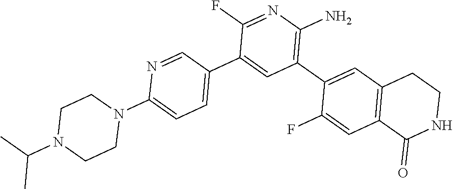 [SMILES: CC(C)N1CCN(c2ccc(-c3cc(-c4cc5c(cc4F)C(=O)NCC5)c(N)nc3F)cn2)CC1] |
| I-227 | 6-(2-amino-5-(3-amino-8- chloro-7-fluoroisoquinolin-6- yl)-6-fluoropyridin-3-yl)-3,4- dihydroisoquinolin-1(2H)- one | 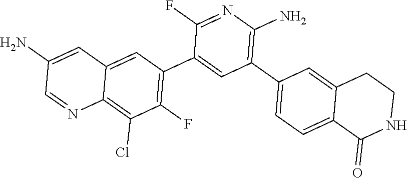 [SMILES: Nc1cnc2c(Cl)c(F)c(-c3cc(-c4ccc5c(c4)CCNC5=O)c(N)nc3F)cc2c1] |
| I-228 | 2-(6-amino-2-fluoro-5-(1- oxo-1,2,3,4- tetrahydroisoquinolin-6- yl)pyridin-3-yl)-6-isopropyl- 5,6-dihydro-4H-pyrazolo[1,5- d][1,4]diazepin-7(8H)-one/ | 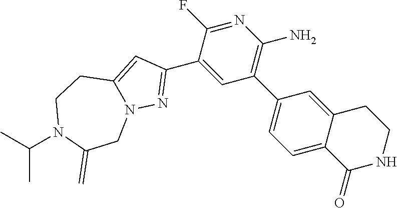 [SMILES: C=C1Cn2nc(-c3cc(-c4ccc5c(c4)CCNC5=O)c(N)nc3F)cc2CCN1C(C)C] |
| I-229 | 2-(6-amino-2-fluoro-5-(1- oxo-1,2,3,4- tetrahydroisoquinolin-6- yl)pyridin-3-yl)-5,6-dihydro- 4H-pyrazolo[1,5- d][1,4]diazepin-7(8H)-one | 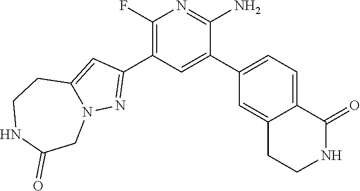 [SMILES: Nc1nc(F)c(-c2cc3n(n2)CC(=O)NCC3)cc1-c1ccc2c(c1)CCNC2=O] |
| I-230 | 6-(2-amino-6-chloro-5-(4-(1- methyl-1,2,3,6- tetrahydropyridin-4- yl)phenyl)yridin-3-yl)-3,4- dihydroisoquinolin-1(2H)- one | 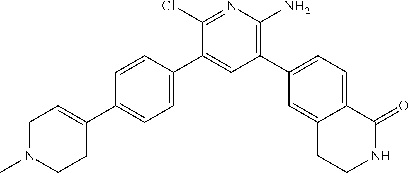 [SMILES: CN1CC=C(c2ccc(-c3cc(-c4ccc5c(c4)CCNC5=O)c(N)nc3Cl)cc2)CC1] |
| I-231 | 6-(2-amino-6-chloro--(4-(1- methylpiperidin-4- yl)phenyl)pyridin-3-yl)-3,4- dihydroisoquinolin-1(2H)- one | 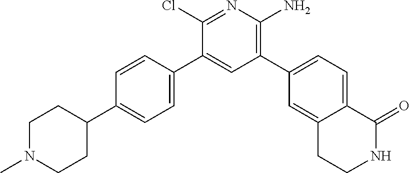 [SMILES: CN1CCC(c2ccc(-c3cc(-c4ccc5c(c4)CCNC5=O)c(N)nc3Cl)cc2)CC1] |
| I-232 | 6-(2-amino-6-chloro-5-(4-(1- methyl-1,2,3,6- tetrahydropyridin-4- yl)phenyl)pyridin-3-yl)-7- fluoro-3,4- dihydroisoquinolin-1(2H)- one | 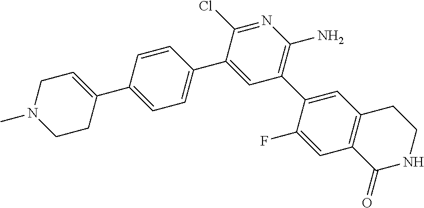 [SMILES: CN1CC=C(c2ccc(-c3cc(-c4cc5c(cc4F)C(=O)NCC5)c(N)nc3Cl)cc2)CC1] |
| I-233 | 6-(2-amino-6-chloro-5-(4-(1- methylpiperidin-4- yl)phenyl)pyridin-3-yl)-7- fluoro-3,4- dihydroisoquinolin-1(2H)- one | 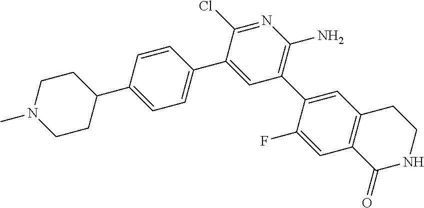 [SMILES: CN1CCC(c2ccc(-c3cc(-c4cc5c(cc4F)C(=O)NCC5)c(N)nc3Cl)cc2)CC1] |
| I-234 | 6-(5′,6-diamino-2-fluoro-4′- methyl-[3,3′-bipyridin]-5-yl)- 3,4-dihydroisoquinolin- 1(2H)-one 2,2,2- trifluoroacetate | 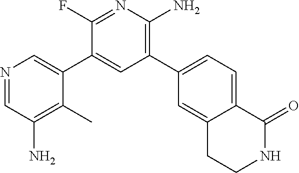 [SMILES: Cc1c(N)cncc1-c1cc(-c2ccc3c(c2)CCNC3=O)c(N)nc1F] |
| I-235 | 6-(2-amino-5-(4-(1- ethylpyrrolidin-3-yl)phenyl)- 6-fluoropyridin-3-yl)-3,4- dihydroisoquinolin-1(2H)- one | 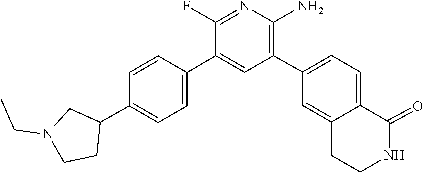 [SMILES: CCN1CCC(c2ccc(-c3cc(-c4ccc5c(c4)CCNC5=O)c(N)nc3F)cc2)C1] |
| I-236 | 6-(2-amino-5-(4-(4-(2,2- difluoroethyl)piperazin-1- yl)phenyl)-6-fluoropyridin-3- yl)-7-fluoro-3,4- dihydroisoquinolin-1(2H)- one | 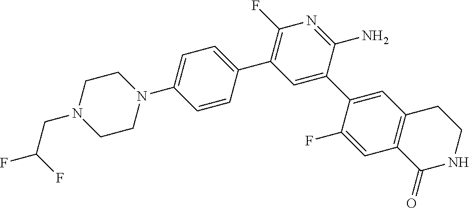 [SMILES: Nc1nc(F)c(-c2ccc(N3CCN(CC(F)F)CC3)cc2)cc1-c1cc2c(cc1F)C(=O)NCC2] |
| I-237 | 6-(2-amino-6-fluoro-5-(4-(4- (2,2,2- trifluoroethyl)piperazin-1- yl)phenyl)pyridin-3-yl)-7- fluoro-3,4- dihydroisoquinolin-1(2H)- one | 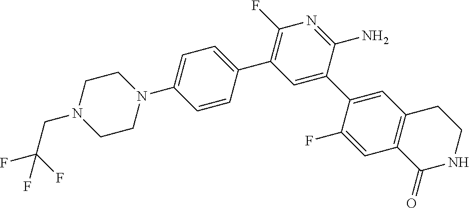 [SMILES: Nc1nc(F)c(-c2ccc(N3CCN(CC(F)(F)F)CC3)cc2)cc1-c1cc2c(cc1F)C(=O)NCC2] |
| I-238 | 6-(2-amino-6-fluoro-5-(4- (piperidin-4- yl)phenyl)pyridin-3-yl)-7- fluoro-3,4- dihydroisoquinolin-1(2H)- one 2,2,2-trifluoroacetate | 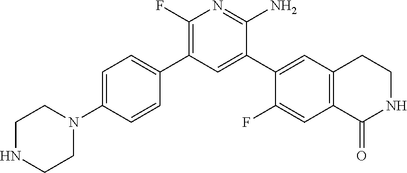 [SMILES: Nc1nc(F)c(-c2ccc(N3CCNCC3)cc2)cc1-c1cc2c(cc1F)C(=O)NCC2] |
| I-239 | 6-(2-amino-6-fluoro-5-(4-(4- fluoro-1-isopropylpiperidin-4- yl)phenyl)pyridin-3-yl)-3,4- dihydroisoquinolin-1(2H)- one | 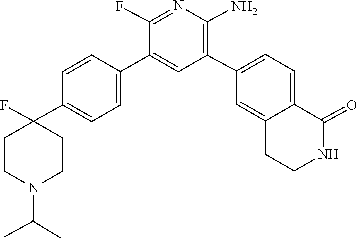 [SMILES: CC(C)N1CCC(F)(c2ccc(-c3cc(-c4ccc5c(c4)CCNC5=O)c(N)nc3F)cc2)CC1] |
| I-240 | 6-(2-amino-6-fluoro-5-(4-(4- (3,3,3- trifluoropropyl)piperazin-1- yl)phenyl)pyridin-3-yl)-3,4- dihydroisoquinolin-1(2H)- one | 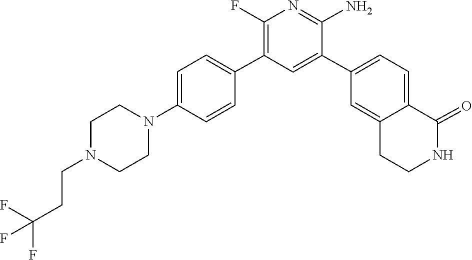 [SMILES: Nc1nc(F)c(-c2ccc(N3CCN(CCC(F)(F)F)CC3)cc2)cc1-c1ccc2c(c1)CCNC2=O] |
| I-241 | 6-(2-amino-6-fluoro-5-(4-(1- (2-methoxyethyl)piperidin-4- yl)phenyl)pyridin-3-yl)-7- fluoro-3,4- dihydroisoquinolin-1(2H)- one | 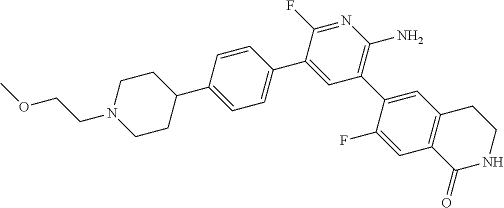 [SMILES: COCCN1CCC(c2ccc(-c3cc(-c4cc5c(cc4F)C(=O)NCC5)c(N)nc3F)cc2)CC1] |
| I-242 | 6-(2-amino-5-(4-(1- (cyclopropylmethyl)piperidin- 4-yl)phenyl)-6-fluoropyridin- 3-yl)-7-fluoro-3,4- dihydroisoquinolin-1(2H)- one | 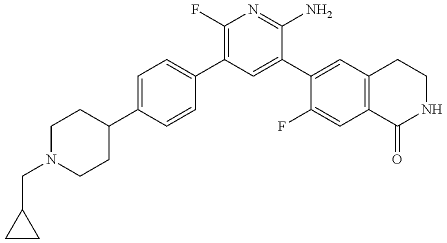 [SMILES: Nc1nc(F)c(-c2ccc(C3CCN(CC4CC4)CC3)cc2)cc1-c1cc2c(cc1F)C(=O)NCC2] |
| I-243 | 6-(2-amino-6-fluoro-5-(4-(4- (3,3,3- trifluoropropyl)piperazin-1- yl)phenyl)pyridin-3-yl)-7- fluoro-3,4- dihydroisoquinolin-1(2H)- one | 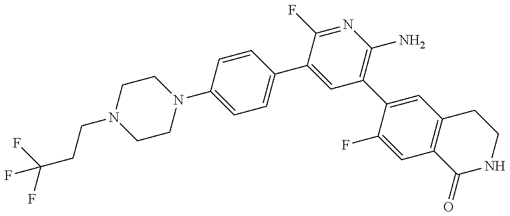 [SMILES: Nc1nc(F)c(-c2ccc(N3CCN(CCC(F)(F)F)CC3)cc2)cc1-c1cc2c(cc1F)C(=O)NCC2] |
| I-244 | 6-(2-amino-6-fluoro-5-(4-(1- isopropylpiperidin-4- yl)phenyl)pyridin-3-yl)-7- fluoro-3,4- dihydroisoquinolin-1(2H)- one | 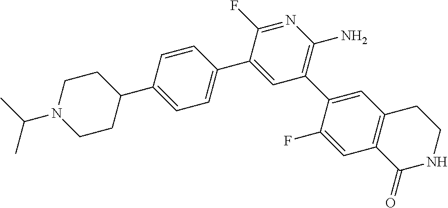 [SMILES: CC(C)N1CCC(c2ccc(-c3cc(-c4cc5c(cc4F)C(=O)NCC5)c(N)nc3F)cc2)CC1] |
| I-245 | 6-(2-amino-5-(4-(1- ethylpiperidin-4-yl)phenyl)-6- fluoropyridin-3-yl)-7-fluoro- 3,4-dihydroisoquinolin- 1(2H)-one | 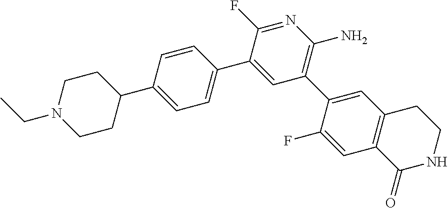 [SMILES: CCN1CCC(c2ccc(-c3cc(-c4cc5c(cc4F)C(=O)NCC5)c(N)nc3F)cc2)CC1] |
| I-246 | 6-(2-amino-6-fluoro-5-(4-(4- methylpiperazin-1- yl)phenyl)pyridin-3-yl)-7- fluoro-3,4- dihydroisoquinolin-1(2H)- one | 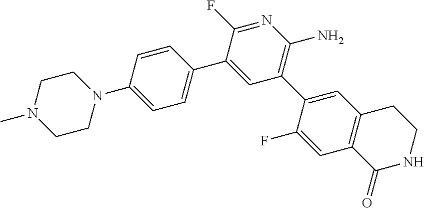 [SMILES: CN1CCN(c2ccc(-c3cc(-c4cc5c(cc4F)C(=O)NCC5)c(N)nc3F)cc2)CC1] |
| I-247 | 6-(2-amino-6-fluoro-5-(3- fluoro-4-(4-methylpiperazin- 1-yl)phenyl)pyridin-3-yl)-7- fluoro-3,4- dihydroisoquinolin-1(2H)- one | 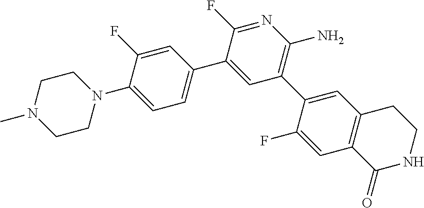 [SMILES: CN1CCN(c2ccc(-c3cc(-c4cc5c(cc4F)C(=O)NCC5)c(N)nc3F)cc2F)CC1] |
| I-248 | 6-(2-amino-6-fluoro-5-(4-(1- (2,2,2-trifluoroethyl)piperidin- 4-yl)phenyl)pyridin-3-yl)-7- fluoro-3,4- dihydroisoquinolin-1(2H)- one | 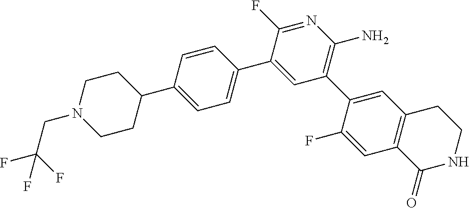 [SMILES: Nc1nc(F)c(-c2ccc(C3CCN(CC(F)(F)F)CC3)cc2)cc1-c1cc2c(cc1F)C(=O)NCC2] |
| I-249 | 6-(2-amino-6-fluoro-5-(4-(4- methylpiperazin-1-l)-3- (trifluoromethyl)phenyl)pyridin- 3-yl)-7-fluoro-3,4- dihydroisoquinolin-1(2H)- one | 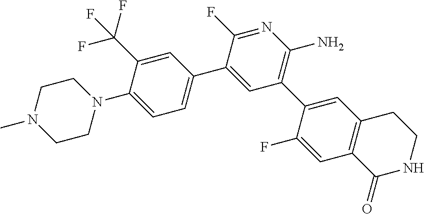 [SMILES: CN1CCN(c2ccc(-c3cc(-c4cc5c(cc4F)C(=O)NCC5)c(N)nc3F)cc2C(F)(F)F)CC1] |
| I-250 | 6-(2-amino-6-fluoro-5-(2- fluoro-5-nitro-4-((3R,5S)- 3,4,5-trimethylpiperazin-1- yl)phenyl)pyridin-3-yl)-3,4- dihydroisoquinolin-1(2H)- one formate | 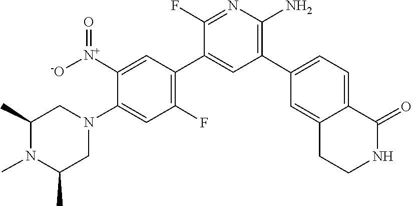 |
| I-251 | 6-(2-amino-6-fluoro-5-(4-(4- fluoro-1-(2- methoxyethyl)piperidin-4- yl)phenyl)pyridin-3-yl)-3,4- dihydroisoquinolin-1(2H)- one | 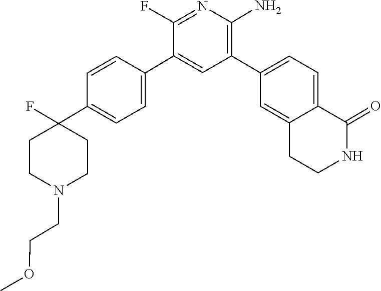 [SMILES: COCCN1CCC(F)(c2ccc(-c3cc(-c4ccc5c(c4)CCNC5=O)c(N)nc3F)cc2)CC1] |
| I-252 | 6-(2-amino-5-(2-chloro-4-(4- methylpiperazin-1- yl)phenyl)-6-fluoropyridin-3- yl)-7-fluoro-3,4- dihydroisoquinolin-1(2H)- one | 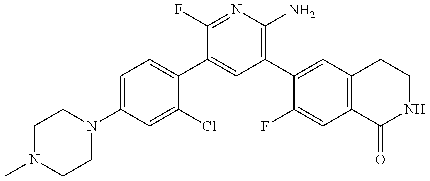 [SMILES: CN1CCN(c2ccc(-c3cc(-c4cc5c(cc4F)C(=O)NCC5)c(N)nc3F)c(Cl)c2)CC1] |
| I-253 | 6-(2-amino-6-fluoro-5-(1- methylindolin-5-yl)pyridin-3- yl)-3,4-dihydroisoquinolin- 1(2H)-one | 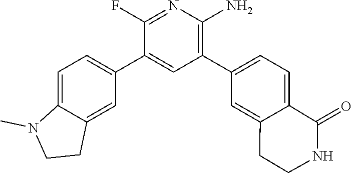 [SMILES: CN1CCc2cc(-c3cc(-c4ccc5c(c4)CCNC5=O)c(N)nc3F)ccc21] |
| I-254 | 6-(2-amino-6-fluoro-5-(2- methyl-1,2,3,4- tetrahydroisoquinolin-6- yl)pyridin-3-yl)-3,4- dihydroisoquinolin-1(2H)- one | 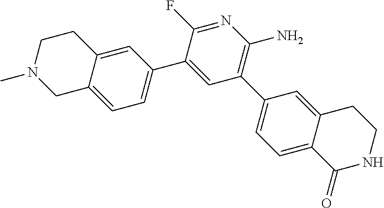 [SMILES: CN1CCc2cc(-c3cc(-c4ccc5c(c4)CCNC5=O)c(N)nc3F)ccc2C1] |
| I-255 | 6-(2-amino-6-fluoro-5- (1,2,3,4- tetrahydroisoquinolin-6- yl)pyridin-3-yl)-3,4- dihydroisoquinolin-1(2H)- one 2,2,2-trifluoroacetate | 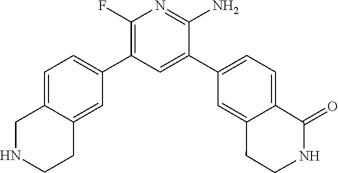 [SMILES: Nc1nc(F)c(-c2ccc3c(c2)CCNC3)cc1-c1ccc2c(c1)CCNC2=O] |
| I-256 | 6-(2-amino-5-(4-(1-(2,2- difluoroethyl)piperidin-4- yl)phenyl)-6-fluoropyridin-3- yl)-7-fluoro-3,4- dihydroisoquinolin-1(2H)- one | 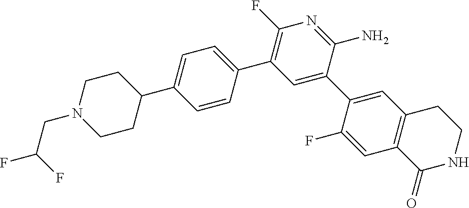 [SMILES: Nc1nc(F)c(-c2ccc(C3CCN(CC(F)F)CC3)cc2)cc1-c1cc2c(cc1F)C(=O)NCC2] |
| I-257 | 6-(2-amino-6-fluoro-5-(4- (morpholinomethyl)phenyl)py- ridin-3-yl)-3,4- dihydroisoquinolin-1(2H)- one | 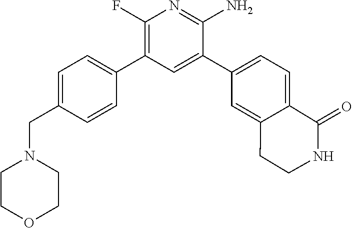 [SMILES: Nc1nc(F)c(-c2ccc(CN3CCOCC3)cc2)cc1-c1ccc2c(c1)CCNC2=O] |
| I-258 | 6-(2-amino-6-fluoro-5-(4-((4- methylpiperazin-1- yl)methyl)phenyl)pyridin-3- yl)-3,4-dihydroisoquinolin- 1(2H)-one | 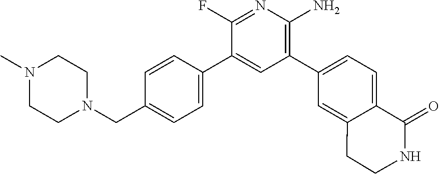 [SMILES: CN1CCN(Cc2ccc(-c3cc(-c4ccc5c(c4)CCNC5=O)c(N)nc3F)cc2)CC1] |
| I-259 | 6-(2-amino-6-fluoro-5-(4-(4- methylpiperazine-1- carbonyl)phenyl)pyridin-3- yl)-3,4-dihydroisoquinolin- 1(2H)-one | 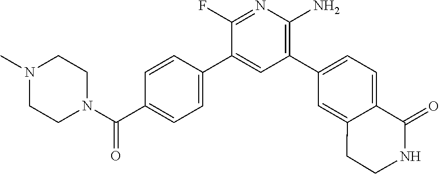 [SMILES: CN1CCN(C(=O)c2ccc(-c3cc(-c4ccc5c(c4)CCNC5=O)c(N)nc3F)cc2)CC1] |
| I-260 | 6-(2-amino-6-fluoro-5-(4-(1- (4-methylpiperazin-1- yl)cyclopropyl)phenyl)pyridin- 3-yl)-3,4-dihydroisoquinolin- 1(2H)-one | 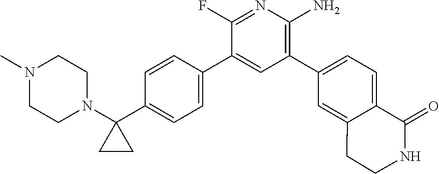 [SMILES: CN1CCN(C2(c3ccc(-c4cc(-c5ccc6c(c5)CCNC6=O)c(N)nc4F)cc3)CC2)CC1] |
| I-261 | 6-(2-amino-6-fluoro-5-(4-((1- isopropylpiperidin-4-yl)oxy)- 3- (trifluoromethyl)phenyl)pyridin- 3-yl)-3,4- dihydroisoquinolin-1(2H)- one | 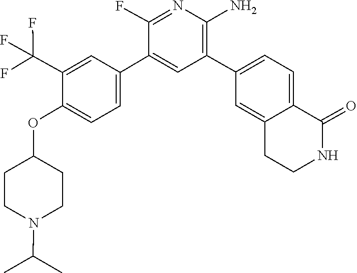 [SMILES: CC(C)N1CCC(Oc2ccc(-c3cc(-c4ccc5c(c4)CCNC5=O)c(N)nc3F)cc2C(F)(F)F)CC1] |
| I-262 | 6-(2-amino-6-fluoro-5-(4-((1- (2-methoxyethyl)piperidin-4- yl)oxy)-3- (trifluoromethyl)phenyl)pyridin- 3-yl)-3,4- dihydroisoquinolin-1(2H)- one | 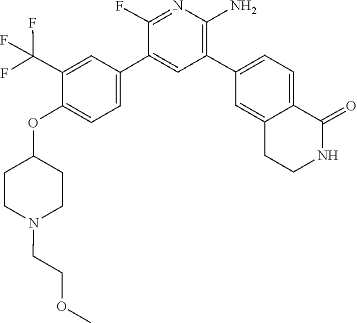 [SMILES: COCCN1CCC(Oc2ccc(-c3cc(-c4ccc5c(c4)CCNC5=O)c(N)nc3F)cc2C(F)(F)F)CC1] |
| I-263 | 6-(2-amino-6-fluoro-5-(4-(4- isopropylpiperazin-1-yl)-3- (trifluoromethyl)phenyl)pyridin- 3-yl)-7-fluoro-3,4- dihydroisoquinolin-1(2H)- one | 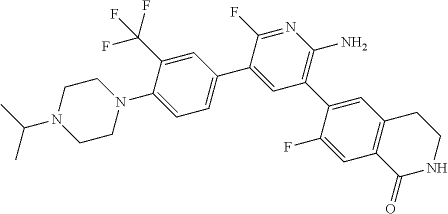 [SMILES: CC(C)N1CCN(c2ccc(-c3cc(-c4cc5c(cc4F)C(=O)NCC5)c(N)nc3F)cc2C(F)(F)F)CC1] |
| I-264 | 6-(2-amino-6-fluoro-5-(4-(4- (2-methoxyethyl)piperazin-1- yl)-3- (trifluoromethyl)phenyl)pyridin- 3-yl)-7-fluoro-3,4- dihydroisoquinolin-1(2H)- one | 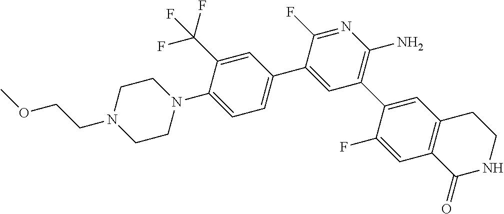 [SMILES: COCCN1CCN(c2ccc(-c3cc(-c4cc5c(cc4F)C(=O)NCC5)c(N)nc3F)cc2C(F)(F)F)CC1] |
| I-265 | 6-(2-amino-6-fluoro-5-(3- (piperazin-1- ylmethyl)phenyl)pyridin-3- yl)-3,4-dihydroisoquinolin- 1(2H)-one formate | 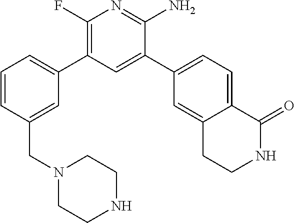 [SMILES: Nc1nc(F)c(-c2cccc(CN3CCNCC3)c2)cc1-c1ccc2c(c1)CCNC2=O] |
| I-266 | 6-(2-amino-6-fluoro-5-(2-(2- methoxyethyl)-1,2,3,4- tetrahydroisoquinolin-6- yl)pyridin-3-yl)-3,4- dihydroisoquinolin-1(2H)- one | 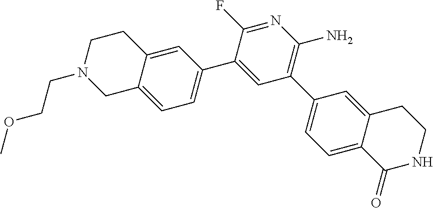 [SMILES: COCCN1CCc2cc(-c3cc(-c4ccc5c(c4)CCNC5=O)c(N)nc3F)ccc2C1] |
| I-267 | 6-(2-amino-6-fluoro-5-(4-((1- isopropylpiperidin-4- yl)oxy)phenyl)pyridin-3-yl)- 3,4-dihydroisoquinolin- 1(2H)-one | 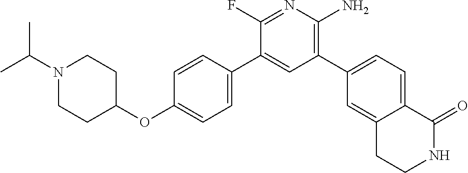 [SMILES: CC(C)N1CCC(Oc2ccc(-c3cc(-c4ccc5c(c4)CCNC5=O)c(N)nc3F)cc2)CC1] |
| I-268 | 6-(2-amino-6-fluoro-5-(3- fluoro-4-((1- isopropylpiperidin-4- yl)oxy)phenyl)pyridin-3-yl)- 3,4-dihydroisoquinolin- 1(2H)-one | 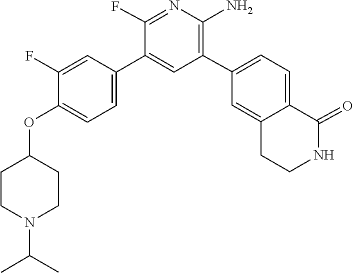 [SMILES: CC(C)N1CCC(Oc2ccc(-c3cc(-c4ccc5c(c4)CCNC5=O)c(N)nc3F)cc2F)CC1] |
| I-269 | 6-(2-amino-6-fluoro-5-(3- fluoro-4-((1-(2- methoxyethyl)piperidin-4- yl)oxy)phenyl)pyridin-3-yl)- 3,4-dihydroisoquinolin- 1(2H)-one | 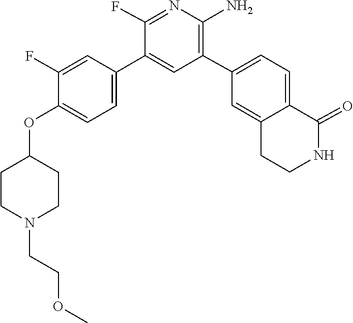 [SMILES: COCCN1CCC(Oc2ccc(-c3cc(-c4ccc5c(c4)CCNC5=O)c(N)nc3F)cc2F)CC1] |
| I-270 | 6-(2-amino-5-(2,3-difluoro-4- ((1-isopropylpiperidin-4- yl)oxy)phenyl)-6- fluoropyridin-3-yl)-3,4- dihydroisoquinolin-1(2H)- one | 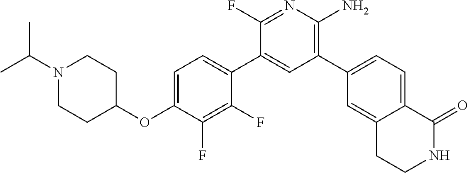 [SMILES: CC(C)N1CCC(Oc2ccc(-c3cc(-c4ccc5c(c4)CCNC5=O)c(N)nc3F)c(F)c2F)CC1] |
| I-271 | 6-(2-amino-5-(2,5-difluoro-4- ((1-isopropylpiperidin-4- yl)oxy)phenyl)-6- fluoropyridin-3-yl)-3,4- dihydroisoquinolin-1(2H)- one | 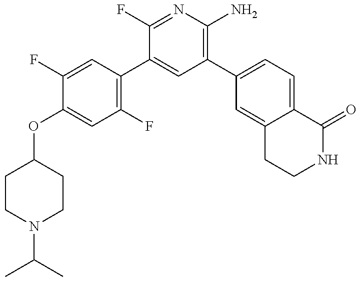 [SMILES: CC(C)N1CCC(Oc2cc(F)c(-c3cc(-c4ccc5c(c4)CCNC5=O)c(N)nc3F)cc2F)CC1] |
| I-272 | 6-(2-amino-6-fluoro-5-(4-((1- isopropylpiperidin-4-yl)oxy)- 2-methoxyphenyl)pyridin-3- yl)-3,4-dihydroisoquinolin- 1(2H)-one | 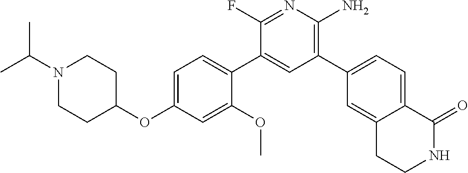 [SMILES: COc1cc(OC2CCN(C(C)C)CC2)ccc1-c1cc(-c2ccc3c(c2)CCNC3=O)c(N)nc1F] |
| I-273 | 6-(2-amino-6-fluoro-5-(4-((1- isopropylpiperidin-4-yl)oxy)- 3-methoxyphenyl)pyridin-3- y)-3,4-dihydroisoquinolin- 1(2H)-one | 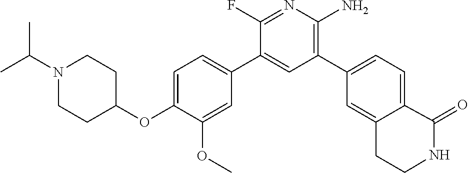 [SMILES: COc1cc(-c2cc(-c3ccc4c(c3)CCNC4=O)c(N)nc2F)ccc1OC1CCN(C(C)C)CC1] |
| I-274 | (S)-6-(2-amino-6-fluoro-5-(4- ((1-isopropylpiperidin-3- yl)oxy)phenyl)pyridin-3-yl)- 3,4-dihydroisoquinolin- 1(2H)-one | 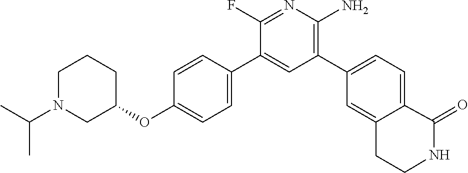 [SMILES: CC(C)N1CCC[C@H](Oc2ccc(-c3cc(-c4ccc5c(c4)CCNC5=O)c(N)nc3F)cc2)C1] |
| I-275 | 6-(2-amino-6-fluoro-5-(7-((1- isopropylpiperidin-4- yl)oxy)benzo[d][1,3]dioxol-4- yl)pyridin-3-yl)-3,4- dihydroisoquinolin-1(2H)- one | 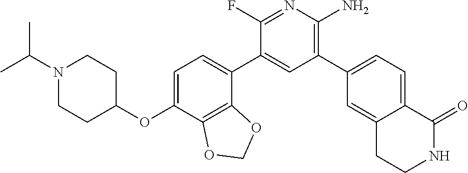 [SMILES: CC(C)N1CCC(Oc2ccc(-c3cc(-c4ccc5c(c4)CCNC5=O)c(N)nc3F)c3c2OCO3)CC1] |
| I-276 | 6-(6-amino-2-fluoro-5-(1- oxo-1,2,3,4- tetrahydroisoquinolin-6- yl)pyridin-3-yl)-3- ((methylamino)methyl)-3,4- dihydroisoquinolin-1(2H)- one | 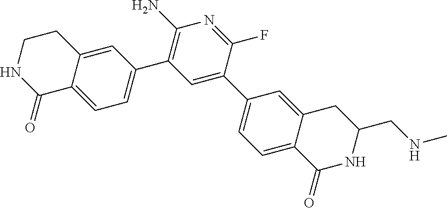 [SMILES: CNCC1Cc2cc(-c3cc(-c4ccc5c(c4)CCNC5=O)c(N)nc3F)ccc2C(=O)N1] |
| I-277 | 6-(2-amino-6-fluoro-5-(4-(4- methyl-1,4-diazepan-1- yl)phenyl)pyridin-3-yl)-3,4- dihydroisoquinolin-1(2H)- one | 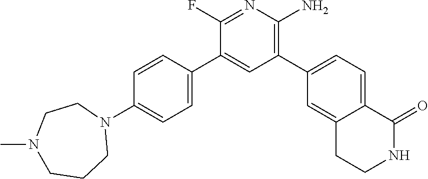 [SMILES: CN1CCCN(c2ccc(-c3cc(-c4ccc5c(c4)CCNC5=O)c(N)nc3F)cc2)CC1] |
| I-278 | 6-(2-amino-6-fluoro-5-(4-(4- isopropyl-3-methylpiperazin- 1-yl)phenyl)pyridin-3-yl)-3,4- dihydroisoquinolin-1(2H)- one | 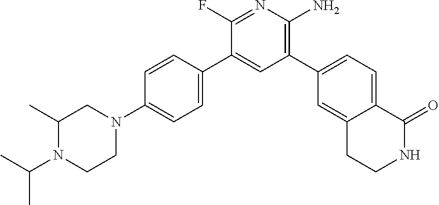 [SMILES: CC(C)N1CCN(c2ccc(-c3cc(-c4ccc5c(c4)CCNC5=O)c(N)nc3F)cc2)CC1C] |
| I-279 | 6-(2-amino-6-fluoro-5-(4-(4- isopropyl-1,4-diazepan-1- yl)phenyl)pyridin-3-yl)-3,4- dihydroisoquinolin-1(2H)- one | 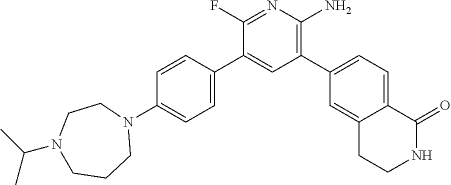 [SMILES: CC(C)N1CCCN(c2ccc(-c3cc(-c4ccc5c(c4)CCNC5=O)c(N)nc3F)cc2)CC1] |
| I-280 | 6-(2-amino-6-fluoro-5-(4- (hexahydropyrrolo[1,2- a]pyrazin-2(1H)- yl)phenyl)pyridin-3-yl)-3,4- dihydroisoquinolin-1(2H)- one | 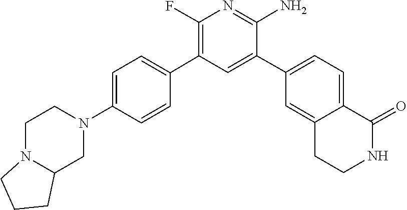 [SMILES: Nc1nc(F)c(-c2ccc(N3CCN4CCCC4C3)cc2)cc1-c1ccc2c(c1)CCNC2=O] |
| I-281 | 6-(2-amino-6-fluoro-5-(4- (hexahydro-1H-pyrrolo[1,2- a][1,4]diazepin-2(3H)- yl)phenyl)pyridin-3-yl)-3,4- dihydroisoquinolin-1(2H)- one | 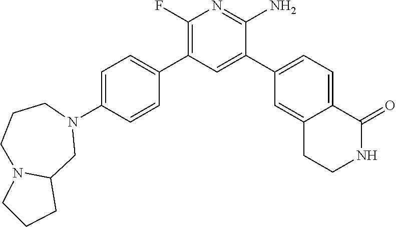 [SMILES: Nc1nc(F)c(-c2ccc(N3CCCN4CCCC4C3)cc2)cc1-c1ccc2c(c1)CCNC2=O] |
| I-282 | (S)-6-(2-amino-5-(4-(2,4- dimethylpiperazin-1- yl)phenyl)-6-fluoropyridin-3- yl)-3,4-dihydroisoquinolin- 1(2H)-one | 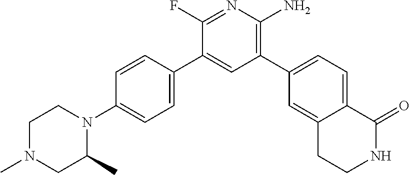 [SMILES: C[C@H]1CN(C)CCN1c1ccc(-c2cc(-c3ccc4c(c3)CCNC4=O)c(N)nc2F)cc1] |
| I-283 | 6-(2-amino-5-(4-(4- (dimethylamino)piperidin-1- yl)phenyl)-6-fluoropyridin-3- yl)-3,4-dihydroisoquinolin- 1(2H)-one | 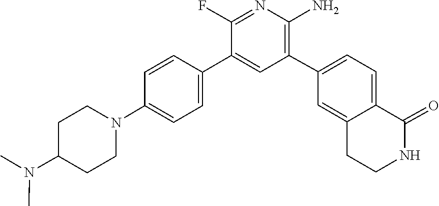 [SMILES: CN(C)C1CCN(c2ccc(-c3cc(-c4ccc5c(c4)CCNC5=O)c(N)nc3F)cc2)CC1] |
| I-284 | tert-butyl (2R,6S)-4-(4-(6- amino-2-fluoro-5-(1-oxo- 1,2,3,4- tetrahydroisoquinolin-6- yl)pyridin-3-yl)phenyl)-2,6- dimethylpiperazine-1- carboxylate | 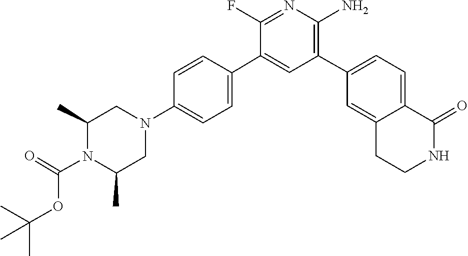 [SMILES: C[C@@H]1CN(c2ccc(-c3cc(-c4ccc5c(c4)CCNC5=O)c(N)nc3F)cc2)C[C@H](C)N1C(=O)OC(C)(C)C] |
| I-285 | 6-(2-amino-6-fluoro-5-(4- morpholinophenyl)pyridin-3- yl)-3-((methylamino)methyl)- 3,4-dihydroisoquinolin- 1(2H)-one | 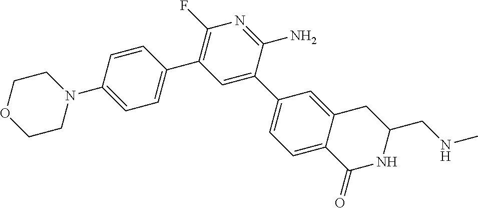 [SMILES: CNCC1Cc2cc(-c3cc(-c4ccc(N5CCOCC5)cc4)c(F)nc3N)ccc2C(=O)N1] |
| I-286 | 6-(2-amino-6-fluoro-5-(4-(4- isopropylpiperazin-1- yl)phenyl)pyridin-3-yl)-3- ((methylamino)methyl)-3,4- dihydroisoquinolin-1(2H)- one | 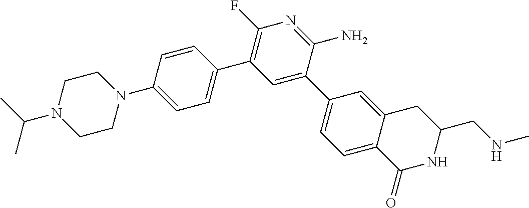 [SMILES: CNCC1Cc2cc(-c3cc(-c4ccc(N5CCN(C(C)C)CC5)cc4)c(F)nc3N)ccc2C(=O)N1] |
| I-287 | 6-(2-amino-6-fluoro-5- (1,2,3,4- tetrahydroisoquinolin-6- yl)pyridin-3-yl)-7-fluoro- 3,4-dihydroisoquinolin- 1(2H)-one 2,2,2- trifluoroacetate | 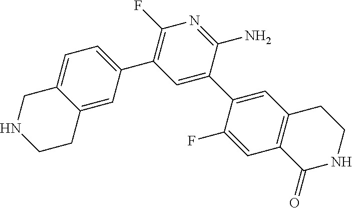 [SMILES: Nc1nc(F)c(-c2ccc3c(c2)CCNC3)cc1-c1cc2c(cc1F)C(=O)NCC2] |
| I-288 | 6-(2-amino-6-fluoro-5-(4-((1- isopropylazetidin-3- yl)oxy)phenyl)pyridin-3-yl)- 3,4-dihydroisoquinolin- 1(2H)-one | 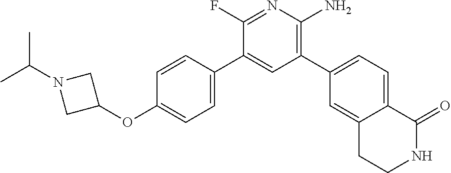 [SMILES: CC(C)N1CC(Oc2ccc(-c3cc(-c4ccc5c(c4)CCNC5=O)c(N)nc3F)cc2)C1] |
| I-289 | 5-(6-amino-2-fluoro-5-(7- fluoro-1-oxo-1,2,3,4- tetrahydroisoquinolin-6- yl)pyridin-3-yl)-2-(piperidin- 4-yloxy)benzonitrile | 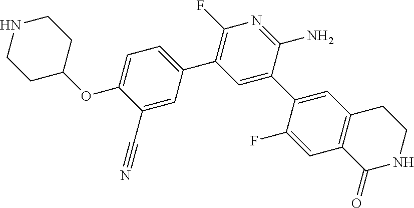 [SMILES: N#Cc1cc(-c2cc(-c3cc4c(cc3F)C(=O)NCC4)c(N)nc2F)ccc1OC1CCNCC1] |
| I-290 | 6-(5-(4-((1-acetylpiperidin-4- yl)oxy)-3-fluorophenyl)-2- amino-6-fluoropyridin-3-yl)- 3,4-dihydroisoquinolin- 1(2H)-one | 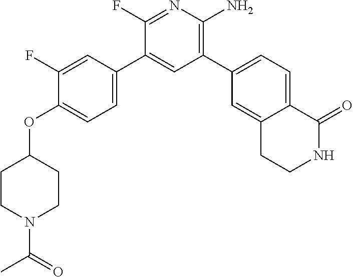 [SMILES: CC(=O)N1CCC(Oc2ccc(-c3cc(-c4ccc5c(c4)CCNC5=O)c(N)nc3F)cc2F)CC1] |
| I-291 | 2-((1-acetylpiperidin-4- yl)oxy)-5-(6-amino-2-fluoro- 5-(7-fluoro-1-oxo-1,2,3,4- tetrahydroisoquinolin-6- yl)pyridin-3-yl)benzonitrile | 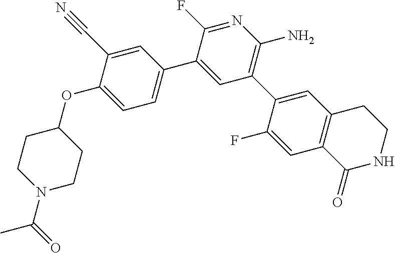 [SMILES: CC(=O)N1CCC(Oc2ccc(-c3cc(-c4cc5c(cc4F)C(=O)NCC5)c(N)nc3F)cc2C#N)CC1] |
| I-292 | 5-(6-amino-2-fluoro-5-(7- fluro-1-oxo-1,2,3,4- tetrahydroisoquinolin-6- yl)pyridin-3-yl)-2-((1- isopropylpiperidin-4- yl)oxy)benzonitrile | 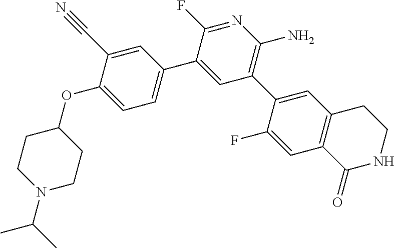 [SMILES: CC(C)N1CCC(Oc2ccc(-c3cc(-c4cc5c(cc4F)C(=O)NCC5)c(N)nc3F)cc2C#N)CC1] |
| I-293 | 5-(6-amino-2-fluoro-5-(7- fluroo-1-oxo-1,2,3,4- tetrahydroisoquinolin-6- yl)pyridin-3-yl)-2-((1-(2,2,2- trifluroethyl)piperidin-4- yl)oxy)benzonitrile | 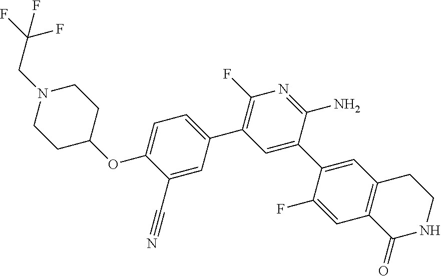 [SMILES: N#Cc1cc(-c2cc(-c3cc4c(cc3F)C(=O)NCC4)c(N)nc2F)ccc1OC1CCN(CC(F)(F)F)CC1] |
| I-294 | 6-(2-amino-6-fluoro-5-(4-(1- isopropyl-4- methoxypiperidin-4- yl)phenyl)pyridin-3-yl)-3,4- dihydroisoquinolin-1(2H)- one | 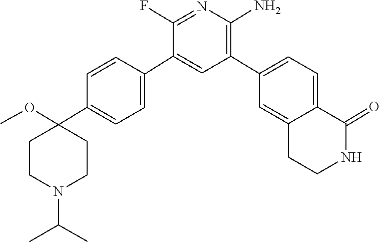 [SMILES: COC1(c2ccc(-c3cc(-c4ccc5c(c4)CCNC5=O)c(N)nc3F)cc2)CCN(C(C)C)CC1] |
| I-295 | 6-(2-amino-6-fluoro-5-(4-(1- isopropyl-1,2,3,6- tetrahydropyridin-4- yl)phenyl)pyridin-3-yl)-7- fluoro-3,4- dihydroisoquinolin-1(2H)- one | 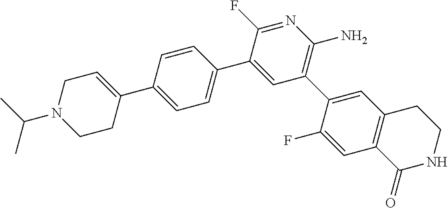 [SMILES: CC(C)N1CC=C(c2ccc(-c3cc(-c4cc5c(cc4F)C(=O)NCC5)c(N)nc3F)cc2)CC1] |
| I-296 | 6-(2-amino-5-(4-(1-ethyl- 1,2,3,6-tetrahydropyridin-4- yl)phenyl)-6-fluoropyridin-3- yl)-7-fluoro-3,4- dihydroisoquinolin-1(2H)- one | 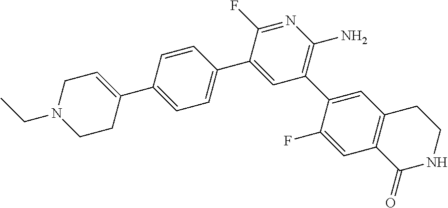 [SMILES: CCN1CC=C(c2ccc(-c3cc(-c4cc5c(cc4F)C(=O)NCC5)c(N)nc3F)cc2)CC1] |
| I-297 | 6-(2-amino-5-(4-(1- (cyclopropylmethyl)-1,2,3,6- tetrahydropyridin-4- yl)phenyl)-6-fluoropyridin-3- yl)-7-fluoro-3,4- dihydroisoquinolin-1(2H)- one | 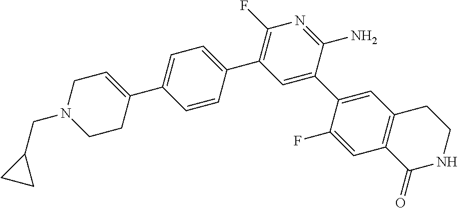 [SMILES: Nc1nc(F)c(-c2ccc(C3=CCN(CC4CC4)CC3)cc2)cc1-c1cc2c(cc1F)C(=O)NCC2] |
| I-298 | 6-(2-amino-5-(4- chlorophenyl)-6- fluoropyridin-3-yl)-3- ((methylamino)methyl)-3,4- dihydroisoquinolin-1(2H)- one | 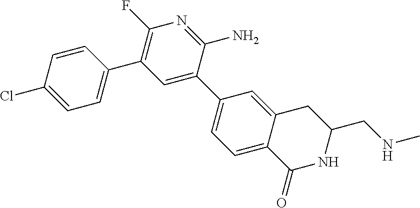 [SMILES: CNCC1Cc2cc(-c3cc(-c4ccc(Cl)cc4)c(F)nc3N)ccc2C(=O)N1] |
| I-299 | 6-(2-amino-6-fluoro-5-(2- isopropyl-1,2,3,4- tetrahydroisoquinolin-6- yl)pyridin-3-yl)-7-fluoro-3,4- dihydroisoquinolin-1(2H)- one | 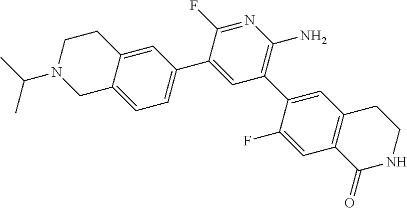 [SMILES: CC(C)N1CCc2cc(-c3cc(-c4cc5c(cc4F)C(=O)NCC5)c(N)nc3F)ccc2C1] |
| I-300 | 6-(2-amino-6-fluoro-5-(2-(2- methoxyethyl)-1,2,3,4- tetrahydroisoquinolin-6- yl)pyridin-3-yl)-7-fluoro-3,4- dihydroisoquinolin-1(2H)- one | 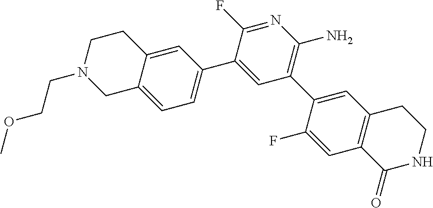 [SMILES: COCCN1CCc2cc(-c3cc(-c4cc5c(cc4F)C(=O)NCC5)c(N)nc3F)ccc2C1] |
| I-301 | 6-(2-amino-6-fluoro-5-(4- ((1R,5S)-3-isopropyl-3- azabicyclo[3.1.0]hexan-1- yl)phenyl)pyridin-3-yl)-8- fluoro-3,4- dihydroisoquinolin-1(2H)- one | 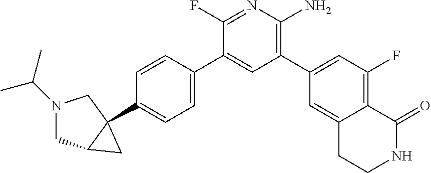 [SMILES: CC(C)N1C[C@H]2C[C@@]2(c2ccc(-c3cc(-c4cc(F)c5c(c4)CCNC5=O)c(N)nc3F)cc2)C1] |
| I-302 | 6-(2-amino-6-fluoro-5-(4-(1- isopropylpiperidin-4- yl)phenyl)pyridin-3-yl)-8- fluoro-3,4- dihydroisoquinolin-1(2H)- one | 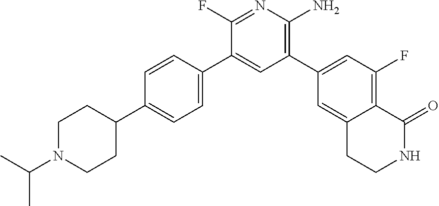 [SMILES: CC(C)N1CCC(c2ccc(-c3cc(-c4cc(F)c5c(c4)CCNC5=O)c(N)nc3F)cc2)CC1] |
| I-303 | 5-(6-amino-2-fluoro-5-(7- fluoro-1-oxo-1,2,3,4- tetrahydroisoquinolin-6- yl)pyridin-3-yl)-2-((1-(2- methoxyethyl)piperidin-4- yl)oxy)benzonitrile | 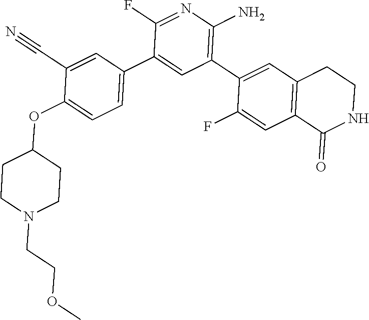 [SMILES: COCCN1CCC(Oc2ccc(-c3cc(-c4cc5c(cc4F)C(=O)NCC5)c(N)nc3F)cc2C#N)CC1] |
| I-304 | 6-(5-(4-((1-acetylpiperidin-4- yl)oxy)-2-methoxyphenyl)-2- amino-6-fluoropyridin-3-yl)- 3,4-dihydroisoquinolin- 1(2H)-one | 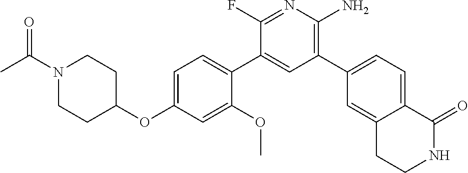 [SMILES: COc1cc(OC2CCN(C(C)=O)CC2)ccc1-c1cc(-c2ccc3c(c2)CCNC3=O)c(N)nc1F] |
| I-305 | 6-(5-(4-((1-acetylpiperidin-4- yl)oxy)-3-methoxyphenyl)-2- amino-6-fluoropyridin-3-yl)- 3,4-dihydroisoquinolin- 1(2H)-one | 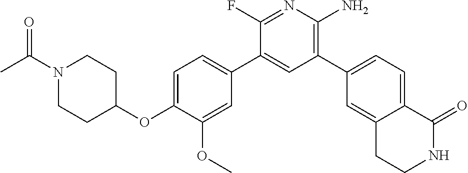 [SMILES: COc1cc(-c2cc(-c3ccc4c(c3)CCNC4=O)c(N)nc2F)ccc1OC1CCN(C(C)=O)CC1] |
| I-306 | 6-(5-(7-((1-acetylpiperidin-4- yl)oxy)benzo[d][1,3]dioxol-4- yl)-2-amino-6-fluoropyridin- 3-yl)-3,4-dihydroisoquinolin- 1(2H)-one | 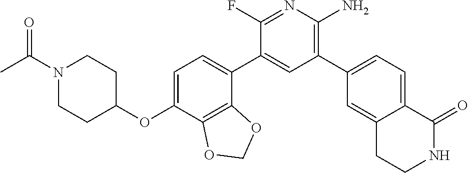 [SMILES: CC(=O)N1CCC(Oc2ccc(-c3cc(-c4ccc5c(c4)CCNC5=O)c(N)nc3F)c3c2OCO3)CC1] |
| I-307 | 6-(2-amino-5-(4-(4- (cyclopropylmethyl)piperazin- 1-yl)phenyl)-6-fluoropyridin- 3-yl)-8-fluoro-3,4- dihydroisoquinolin-1(2H)- one | 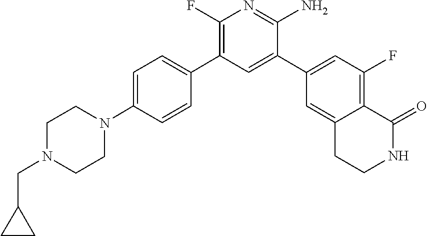 [SMILES: Nc1nc(F)c(-c2ccc(N3CCN(CC4CC4)CC3)cc2)cc1-c1cc(F)c2c(c1)CCNC2=O] |
| I-308 | 6-(2-amino-5-(4-(1- (cyclopropylmethyl)piperidin- 4-yl)phenyl)-6-fluoropyridin- 3-yl)-8-fluoro-3,4- dihydroisoquinolin-1(2H)- one | 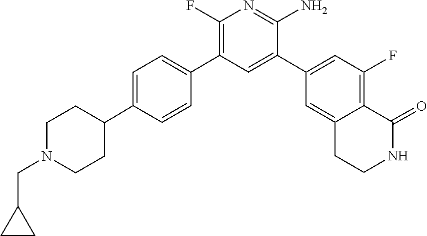 [SMILES: Nc1nc(F)c(-c2ccc(C3CCN(CC4CC4)CC3)cc2)cc1-c1cc(F)c2c(c1)CCNC2=O] |
| I-309 | 6-(2-amino-6-fluoro-5-(4- morpholinophenyl)pyridin-3- yl)-8-fluoro-3,4- dihydroisoquinolin-1(2H)- one | 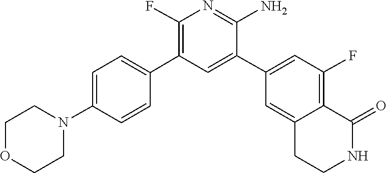 [SMILES: Nc1nc(F)c(-c2ccc(N3CCOCC3)cc2)cc1-c1cc(F)c2c(c1)CCNC2=O] |
| I-310 | 6-(2-amino-6-fluoro-5-(4-(4- isopropylpiperazin-1- yl)phenyl)pyridin-3-yl)-3- ((dimethylamino)methyl)-3,4- dihydroisoquinolin-1(2H)- one | 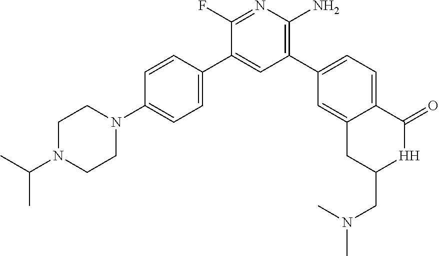 |
| I-311 | 6-(2-amino-6-fluoro-5-(4- morpholinophenyl)pyridin-3- yl)-3- ((dimethylamino)methyl)-3,4- dihydroisoquinolin-1(2H)- one | 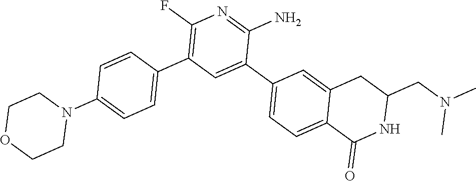 [SMILES: CN(C)CC1Cc2cc(-c3cc(-c4ccc(N5CCOCC5)cc4)c(F)nc3N)ccc2C(=O)N1] |
| I-312 | 6-(2-amino-5-(4- chlorophenyl)-6- fluoropyridin-3-yl)-3- ((dimethylamino)methyl)-3,4- dihydroisoquinolin-1(2H)- one | 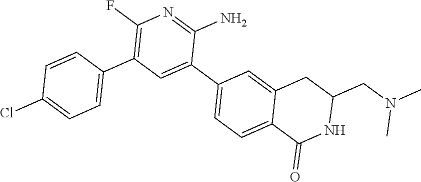 [SMILES: CN(C)CC1Cc2cc(-c3cc(-c4ccc(Cl)cc4)c(F)nc3N)ccc2C(=O)N1] |
| I-313 | 6-(6-amino-2-fluoro-2′- methyl-[3,4′-bipyridin]-5-yl)- 7-fluoro-3,4- dihydroisoquinolin-1(2H)- one | 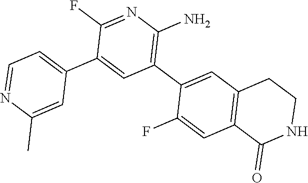 [SMILES: Cc1cc(-c2cc(-c3cc4c(cc3F)C(=O)NCC4)c(N)nc2F)ccn1] |
| I-314 | 6-(6-amino-2′- (cyclopropylmethoxy)-2- fluoro-[3,4′-bipyridin]-5-yl)-7- fluoro-3,4- dihydroisoquinolin-1(2H)- one | 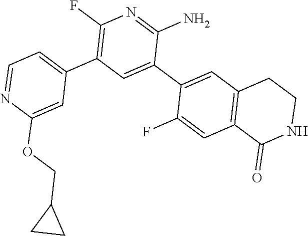 [SMILES: Nc1nc(F)c(-c2ccnc(OCC3CC3)c2)cc1-c1cc2c(cc1F)C(=O)NCC2] |
| I-315 | 6-(2-amino-5-(3- (cyclopropylmethoxy)phenyl)- 6-fluoropyridin-3-yl)-7- fluoro-3,4- dihydroisoquinolin-1(2H)- one | 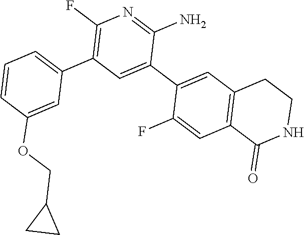 [SMILES: Nc1nc(F)c(-c2cccc(OCC3CC3)c2)cc1-c1cc2c(cc1F)C(=O)NCC2] |
| I-316 | 6-(2-amino-5-(4- (cyclopropylmethoxy)phenyl)- 6-fluoropyridin-3-yl)-7- fluoro-3,4- dihydroisoquinolin-1(2H)- one | 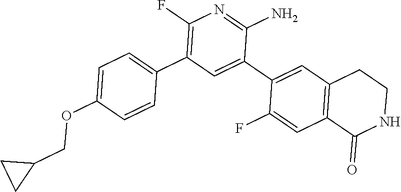 [SMILES: Nc1nc(F)c(-c2ccc(OCC3CC3)cc2)cc1-c1cc2c(cc1F)C(=O)NCC2] |
| I-317 | 6-(2-amino-5-(4-((1-(2- cyclopropylacetyl)azetidin-3- yl)oxy)phenyl)-6- fluoropyridin-3-yl)-3,4- dihydroisoquinolin-1(2H)- one | 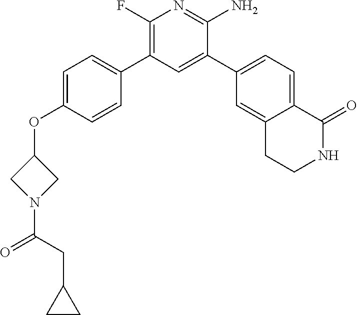 [SMILES: Nc1nc(F)c(-c2ccc(OC3CN(C(=O)CC4CC4)C3)cc2)cc1-c1ccc2c(c1)CCNC2=O] |
| I-318 | 6-(2-amino-6-fluoro-5-(4-((1- (oxetan-3-yl)azetidin-3- yl)oxy)phenyl)pyridin-3-yl)- 3,4-dihydroisoquinolin- 1(2H)-one | 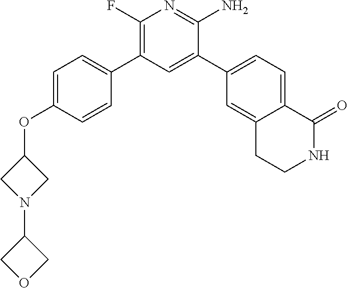 [SMILES: Nc1nc(F)c(-c2ccc(OC3CN(C4COC4)C3)cc2)cc1-c1ccc2c(c1)CCNC2=O] |
| I-319 | 6-(2-amino-5-(4-((3R,5S)- 3,5-dimethylpiperazin-1- yl)phenyl)-6-fluoropyridin-3- yl)-3,4-dihydroisoquinolin- 1(2H)-one | 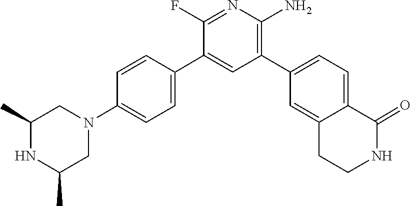 [SMILES: C[C@@H]1CN(c2ccc(-c3cc(-c4ccc5c(c4)CCNC5=O)c(N)nc3F)cc2)C[C@H](C)N1] |
| I-320 | 6-(2-amino-5-(4-((1S,5R)-3- (cyclopropylmethyl)-3- azabicyclo[3.1.0]hexan-1- yl)phenyl)-6-fluoropyridin-3- yl)-8-fluoro-3,4- dihydroisoquinolin-1(2H)- one formate | 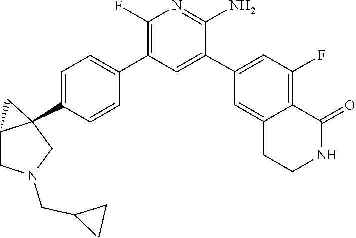 [SMILES: CCC(C)CN1C[C@@H]2C[C@]2(c2ccc(-c3cc(-c4cc(F)c5c(c4)CCNC5=O)c(N)nc3F)cc2)C1] |
| I-321 | 6-(2-amino-5-(4-((1- (cyclopropylmethyl)azetidin- 3-yl)oxy)phenyl)-6- fluoropyridin-3-yl)-3,4- dihydroisoquinolin-1(2H)- one | 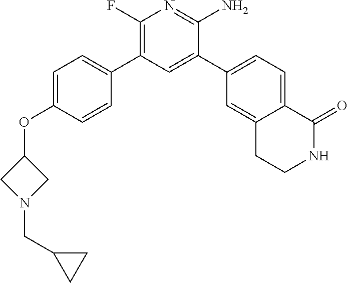 [SMILES: Nc1nc(F)c(-c2ccc(OC3CN(CC4CC4)C3)cc2)cc1-c1ccc2c(c1)CCNC2=O] |
| I-322 | 6-(2-amino-5-(4-((1- (cyclopropylmethyl)piperidin- 4-yl)oxy)phenyl)-6- fluoropyridin-3-yl)-3,4- dihydroisoquinolin-1(2H)- one | 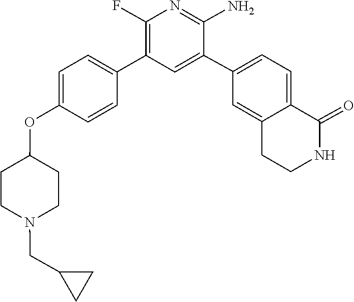 [SMILES: Nc1nc(F)c(-c2ccc(OC3CCN(CC4CC4)CC3)cc2)cc1-c1ccc2c(c1)CCNC2=O] |
| I-323 | 6-(2-amino-5-(4-((1-(2- cyclopropylacetyl)piperidin- 4-yl)oxy)phenyl)-6- fluoropyridin-3-yl)-7-fluoro- 3,4-dihydroisoquinolin- 1(2H)-one | 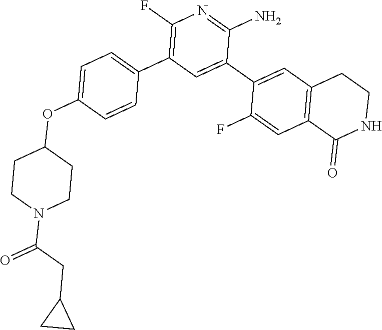 [SMILES: Nc1nc(F)c(-c2ccc(OC3CCN(C(=O)CC4CC4)CC3)cc2)cc1-c1cc2c(cc1F)C(=O)NCC2] |
| I-324 | 6-(2-amino-5-(4-((1- (cyclopropylmethyl)piperidin- 4-yl)oxy)phenyl)-6- fluoropyridin-3-yl)-7-fluroo- 3,4-dihydroisoquinolin- 1(2H)-one | 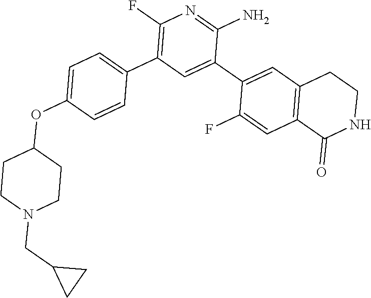 [SMILES: Nc1nc(F)c(-c2ccc(OC3CCN(CC4CC4)CC3)cc2)cc1-c1cc2c(cc1F)C(=O)NCC2] |
| I-325 | 6-(2-amino-6-fluoro-5-(4-((1- (oxetan-3-yl)piperidin-4- yl)oxy)phenyl)pyridin-3-yl)-7- fluoro-3,4- dihydroisoquinolin-1(2H)- one | 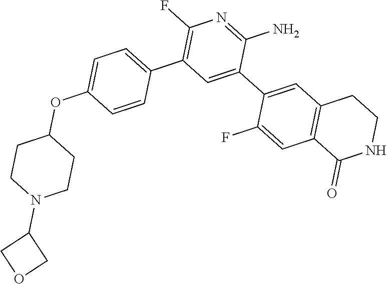 [SMILES: Nc1nc(F)c(-c2ccc(OC3CCN(C4COC4)CC3)cc2)cc1-c1cc2c(cc1F)C(=O)NCC2] |
| I-326 | 6-(5-(4-((1-acetylazetidin-3- yl)oxy)phenyl)-2-amino-6- fluoropyridin-3-yl)-3,4- dihydroisoquinolin-1(2H)- one | 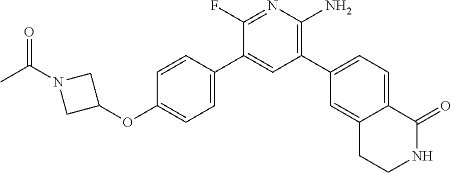 [SMILES: CC(=O)N1CC(Oc2ccc(-c3cc(-c4ccc5c(c4)CCNC5=O)c(N)nc3F)cc2)C1] |
| I-327 | 6-(5-(4-((1-acetylpiperidin-4- yl)oxy)phenyl)-2-amino-6- fluoropyridin-3-yl)-7-fluoro- 3,4-dihydroisoquinolin- 1(2H)-one | 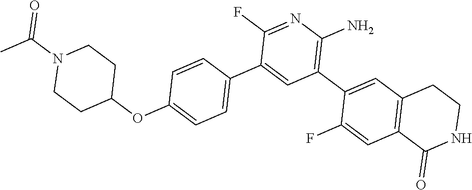 [SMILES: CC(=O)N1CCC(Oc2ccc(-c3cc(-c4cc5c(cc4F)C(=O)NCC5)c(N)nc3F)cc2)CC1] |
| I-328 | 6-(2-amino-6-fluoro-5-(2- isopropyl-1,2,3,4- tetrahydroisoquinolin-6- yl)pyridin-3-yl)-3,4- dihydroisoquinolin-1(2H)- one | 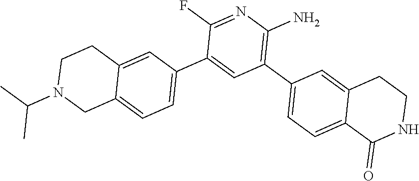 [SMILES: CC(C)N1CCc2cc(-c3cc(-c4ccc5c(c4)CCNC5=O)c(N)nc3F)ccc2C1] |
| I-329 | 6-(2-amino-6-fluoro-5-(3-((4- (2-methoxyethyl)piperazin-1- yl)methyl)phenyl)pyridin-3- yl)-3,4-dihydroisoquinolin- 1(2H)-one | 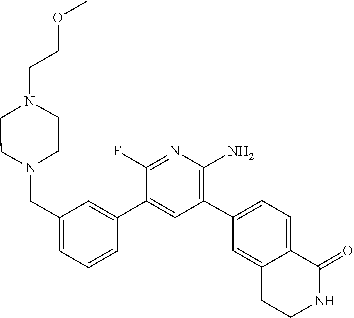 [SMILES: COCCN1CCN(Cc2cccc(-c3cc(-c4ccc5c(c4)CCNC5=O)c(N)nc3F)c2)CC1] |
| I-330 | 6-(2-amino-6-fluoro-5-(3-((4- isopropylpiperazin-1- yl)methyl)phenyl)pyridin-3- yl)-3,4-dihydroisoquinolin- 1(2H)-one | 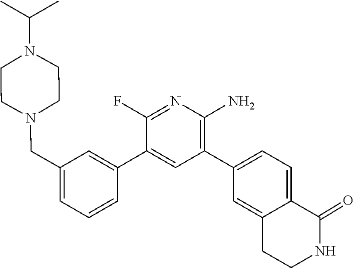 [SMILES: CC(C)N1CCN(Cc2cccc(-c3cc(-c4ccc5c(c4)CCNC5=O)c(N)nc3F)c2)CC1] |
| I-331 | 6-(2-amino-6-fluoro-5-(3-(4- methylpiperazine-1- carbonyl)phenyl)pyridin-3- yl)-3,4-dihydroisoquinolin- 1(2H)-one | 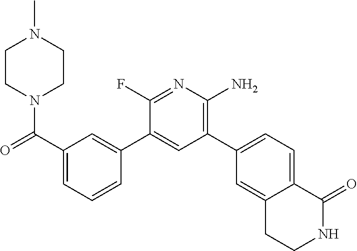 [SMILES: CN1CCN(C(=O)c2cccc(-c3cc(-c4ccc5c(c4)CCNC5=O)c(N)nc3F)c2)CC1] |
| I-332 | 6-(2-amino-5-(3-amino-4-(4- methylpiperazin-1- yl)phenyl)-6-fluoropyridin-3- yl)-3,4-dihydroisoquinolin- 1(2H)-one | 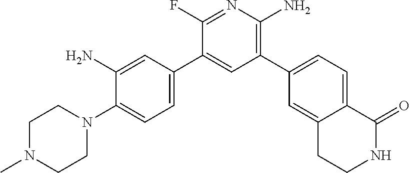 [SMILES: CN1CCN(c2ccc(-c3cc(-c4ccc5c(c4)CCNC5=O)c(N)nc3F)cc2N)CC1] |
| I-333 | 6,6′-(2-amino-6- fluoropyridine-3,5- diyl)bis(3,4- dihydroisoquinolin-1(2H)- one) | 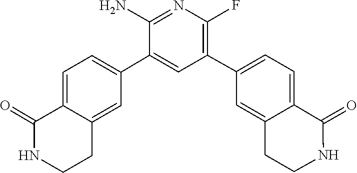 [SMILES: Nc1nc(F)c(-c2ccc3c(c2)CCNC3=O)cc1-c1ccc2c(c1)CCNC2=O] |
| I-334 | 6-(2-amino-6-fluoro-5- (1,2,3,4- tetrahydroisoquinolin-7- yl)pyridin-3-yl)-3,4- dihydroisoquinolin-1(2H)- one 2,2,2-trifluoroacetate | 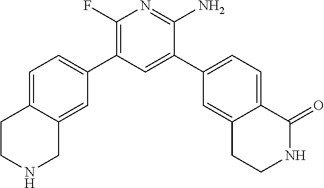 [SMILES: Nc1nc(F)c(-c2ccc3c(c2)CNCC3)cc1-c1ccc2c(c1)CCNC2=O] |
| I-335 | 6-(2-amino-6-fluoro-5-(2- isopropyl-1,2,3,4- tetrahydroisoquinolin-7- yl)pyridin-3-yl)-3,4- dihydroisoquinolin-1(2H)- one | 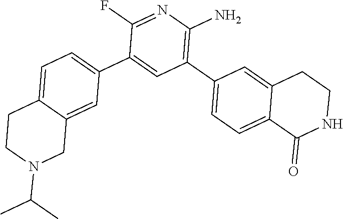 [SMILES: CC(C)N1CCc2ccc(-c3cc(-c4ccc5c(c4)CCNC5=O)c(N)nc3F)cc2C1] |
| I-336 | 6-(2-amino-6-fluoro-5-(4-(4- isopropylpiperazin-1- yl)phenyl)pyridin-3-yl)-8- fluoro-3,4- dihydroisoquinolin-1(2H)- one | 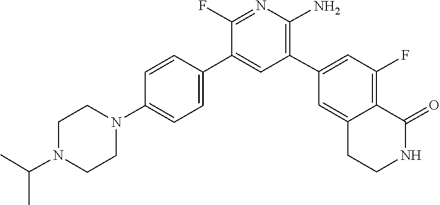 [SMILES: CC(C)N1CCN(c2ccc(-c3cc(-c4cc(F)c5c(c4)CCNC5=O)c(N)nc3F)cc2)CC1] |
| I-337 | 6-(2-amino-5-(4-((1-(2- cyclopropylacetyl)piperidin- 4-yl)oxy)phenyl)-6- fluoropyridin-3-yl)-3,4- dihydroisoquinolin-1(2H)- one | 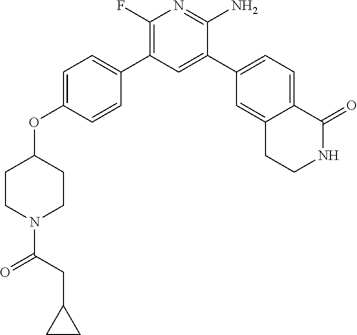 [SMILES: Nc1nc(F)c(-c2ccc(OC3CCN(C(=O)CC4CC4)CC3)cc2)cc1-c1ccc2c(c1)CCNC2=O] |
| I-338 | 6-(2-amino-6-fluoro-5-(4-((1- (oxetan-3-yl)piperidin-4- yl)oxy)phenyl)pyridin-3-yl)- 3,4-dihydroisoquinolin- 1(2H)-one | 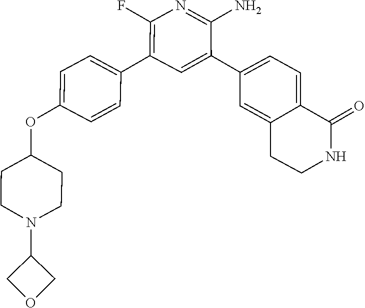 [SMILES: Nc1nc(F)c(-c2ccc(OC3CCN(C4COC4)CC3)cc2)cc1-c1ccc2c(c1)CCNC2=O] |
| I-339 | 6-(2-amino-5-(4-(azetidin-3- yloxy)phenyl)-6- fluoropyridin-3-yl)-7-fluoro- 3,4-dihydroisoquinolin- 1(2H)-one 2,2,2- trifluoroacetate | 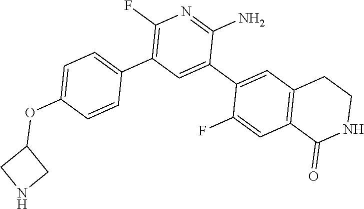 [SMILES: Nc1nc(F)c(-c2ccc(OC3CNC3)cc2)cc1-c1cc2c(cc1F)C(=O)NCC2] |
| I-340 | 6-(2-amino-6-fluoro-5-(4-(3- fluoroazetidin-3- yl)phenyl)pyridin-3-yl)-3,4- dihydroisoquinolin-1(2H)- one 2,2,2-trifluoroacetate | 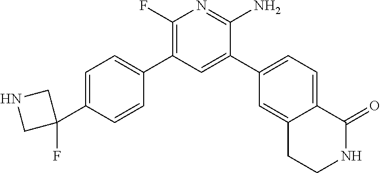 [SMILES: Nc1nc(F)c(-c2ccc(C3(F)CNC3)cc2)cc1-c1ccc2c(c1)CCNC2=O] |
| I-341 | 6-(5-(4-((1-acetylazetidin-3- yl)oxy)phenyl)-2-amino-6- fluoropyridin-3-yl)-7-fluoro- 3,4-dihydroisoquinolin- 1(2H)-one | 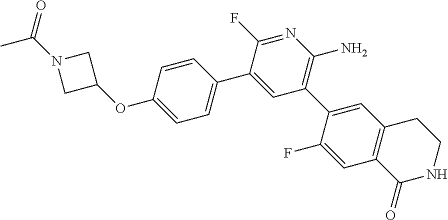 [SMILES: CC(=O)N1CC(Oc2ccc(-c3cc(-c4cc5c(cc4F)C(=O)NCC5)c(N)nc3F)cc2)C1] |
| I-342 | 6-(5-(4-(1-acetyl-3- fluoroazetidin-3-yl)phenyl)-2- amino-6-fluoropyridin-3-yl)- 3,4-dihydroisoquinolin- 1(2H)-one | 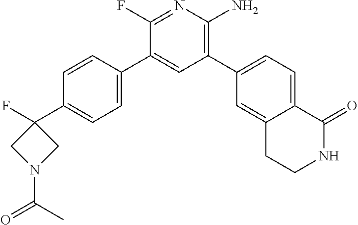 [SMILES: CC(=O)N1CC(F)(c2ccc(-c3cc(-c4ccc5c(c4)CCNC5=O)c(N)nc3F)cc2)C1] |
| I-343 | 6-(2-amino-6-fluoro-5-(4- ((1R,5S)-3-methyl-3- azabicyclo[3.1.0]hexan-1- yl)phenyl)pyridin-3-yl)-7- fluoro-3,4- dihydroisoquinolin-1(2H)- one | 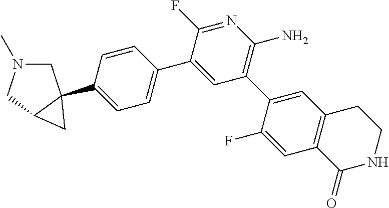 [SMILES: CN1C[C@H]2C[C@@]2(c2ccc(-c3cc(-c4cc5c(cc4F)C(=O)NCC5)c(N)nc3F)cc2)C1] |
| I-344 | 6-(2-amino-6-fluoro-5-(4- ((1S,5R)-3-methyl-3- azabicyclo[3.1.0]hexan-1- yl)phenyl)pyridin-3-yl)-7- fluoro-3,4- dihydroisoquinolin-1(2H)- one | 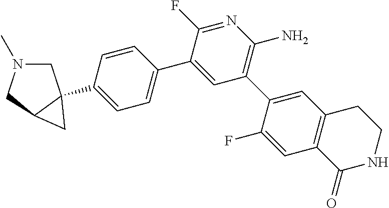 [SMILES: CN1C[C@@H]2C[C@]2(c2ccc(-c3cc(-c4cc5c(cc4F)C(=O)NCC5)c(N)nc3F)cc2)C1] |
| I-345 | 6-(2-amino-5-(2,3-difluoro-4- (4-isopropylpiperazin-1- yl)phenyl)-6-fluoropyridin-3- yL)-3,4-dihydroisoquinolin- 1(2H)-one | 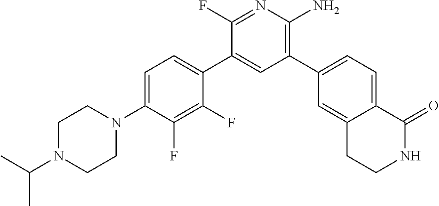 [SMILES: CC(C)N1CCN(c2ccc(-c3cc(-c4ccc5c(c4)CCNC5=O)c(N)nc3F)c(F)c2F)CC1] |
| I-346 | 6-(2-amino-5-(4-(1,1- dioxidothiomorpholino)phenyl)- 6-fluoropyridin-3-yl)-7- fluoro-3,4- dihydroisoquinolin-1(2H)- one | 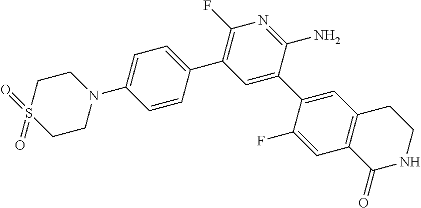 [SMILES: Nc1nc(F)c(-c2ccc(N3CCS(=O)(=O)CC3)cc2)cc1-c1cc2c(cc1F)C(=O)NCC2] |
| I-347 | 6-(2-amino-6-fluoro-5-(4- thiomorpholinophenyl)pyridin- 3-yl)-7-fluoro-3,4- dihydroisoquinolin-1(2H)- one | 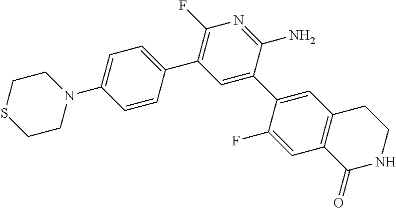 [SMILES: Nc1nc(F)c(-c2ccc(N3CCSCC3)cc2)cc1-c1cc2c(cc1F)C(=O)NCC2] |
| I-348 | (3aS,4S,6aR)-4-(5-(4-(4-(6- amino-2-fluoro-5-(1-oxo- 1,2,3,4- tetrahydroisoquinolin-6- yl)pyridin-3- yl)phenoxy)piperidin-1-yl)-5- oxopentyl)tetrahydro-1H- thieno[3,4-d]imidazol-2(3H)- one | 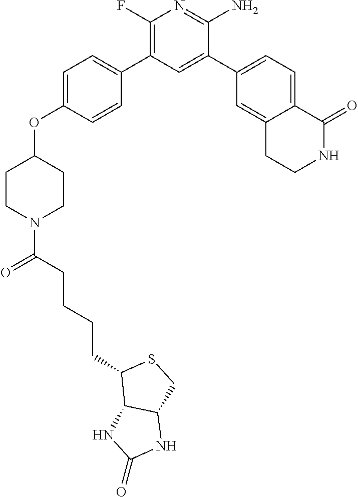 [SMILES: Nc1nc(F)c(-c2ccc(OC3CCN(C(=O)CCCC[C@@H]4SC[C@@H]5NC(=O)N[C@@H]54)CC3)cc2)cc1-c1ccc2c(c1)CCNC2=O] |
| I-349 | 6-(2-amino-6-fluoro-5-(4-(4- isoproylpiperazin-1- yl)phenyl)pyridin-3-yl)-7- chloro-3,4- dihydroisoquinolin-1(2H)- one | 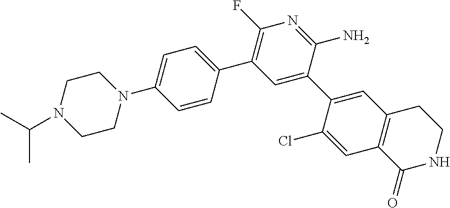 [SMILES: CC(C)N1CCN(c2ccc(-c3cc(-c4cc5c(cc4Cl)C(=O)NCC5)c(N)nc3F)cc2)CC1] |
| I-350 | 6-(2-amino-6-fluoro-5-(4-(1- (2-methoxyethyl)piperidin-4- yl)phenyl)pyridin-3-yl)-8- fluoro-3,4- dihydroisoquinolin-1(2H)- one | 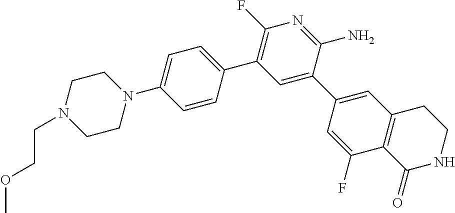 [SMILES: COCCN1CCN(c2ccc(-c3cc(-c4cc(F)c5c(c4)CCNC5=O)c(N)nc3F)cc2)CC1] |
| I-351 | (R)-6-(2-amino-6-fluoro-5-(4- (2- isopropylmorpholino)phenyl) pyridin-3-yl)-8-fluoro-3,4- dihydroisoquinolin-1(2H)- one | 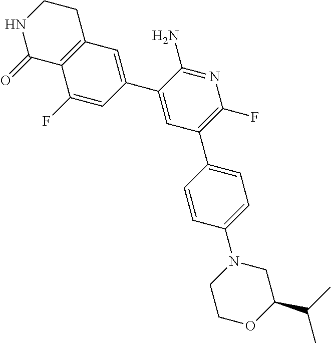 [SMILES: CC(C)[C@@H]1CN(c2ccc(-c3cc(-c4cc(F)c5c(c4)CCNC5=O)c(N)nc3F)cc2)CCO1] |
| I-352 | 6-(2-amino-6-fluoro-5-(4- ((1R,5S)-3-methyl-3- azabicyclo[3.1.0]hexan-1- yl)phenyl)pyridin-3-yl)-8- fluoro-3,4- dihydroisoquinolin-1(2H)- one | 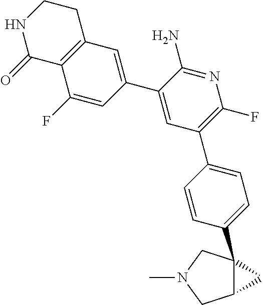 |
| I-353 | 6-(2-amino-6-fluoro-5-(4- ((1S,5R)-3-methyl-3- azabicyclo[3.1.0]hexan-1- yl)phenyl)pyridin-3-yl)-8- fluoro-3,4- dihydroisoquinolin-1(2H)- one | 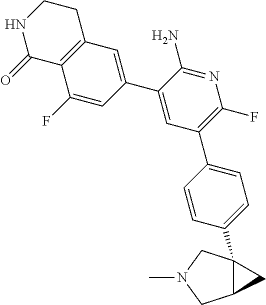 [SMILES: CN1C[C@@H]2C[C@]2(c2ccc(-c3cc(-c4cc(F)c5c(c4)CCNC5=O)c(N)nc3F)cc2)C1] |
| I-354 | 6-(2-amino-6-fluoro-5-(4-(4- isopropylpiperazin-1- yl)phenyl)pyridin-3-yl)-8- methoxy-3,4- dihydroisoquinolin-1(2H)- one | 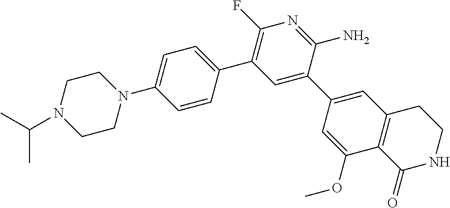 [SMILES: COc1cc(-c2cc(-c3ccc(N4CCN(C(C)C)CC4)cc3)c(F)nc2N)cc2c1C(=O)NCC2] |
| I-355 | 6-(2-amino-6-fluoro-5-(4-(4- isopropylpiperazin-1- yl)phenyl)pyridin-3-yl)-8- (dimethylamino)-3,4- dihydroisoquinolin-1(2H)- one | 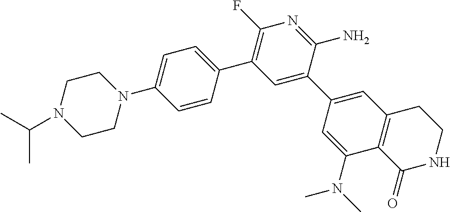 [SMILES: CC(C)N1CCN(c2ccc(-c3cc(-c4cc5c(c(N(C)C)c4)C(=O)NCC5)c(N)nc3F)cc2)CC1] |
| I-356 | 6-(2-amino-6-fluoro-5-(4-(4- isopropylpiperazin-1- yl)phenyl)pyridin-3-yl)-8- (methylamino)-3,4- dihydroisoquinolin-1(2H)- one | 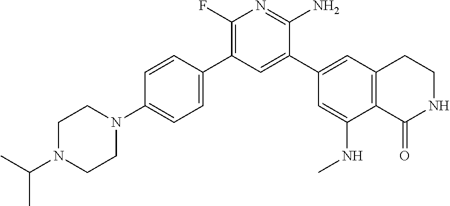 [SMILES: CNc1cc(-c2cc(-c3ccc(N4CCN(C(C)C)CC4)cc3)c(F)nc2N)cc2c1C(=O)NCC2] |
| I-357 | 8-amino-6-(2-amino-6- fluoro-5-(4-(4- isopropylpiperazin-1- yl)phenyl)pyridin-3-yl)-3,4- dihydroisoquinolin-1(2H)- one | 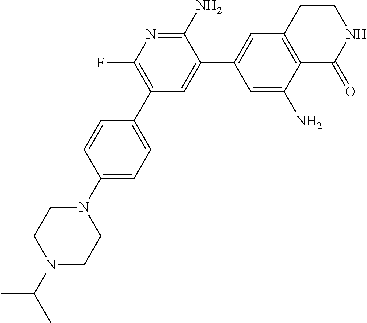 [SMILES: CC(C)N1CCN(c2ccc(-c3cc(-c4cc(N)c5c(c4)CCNC5=O)c(N)nc3F)cc2)CC1] |
| I-358 | 8-(2-amino-6-fluoro-5-(4-(4- isopropylpiperazin-1- yl)phenyl)pyridin-3-yl)-3,4- dihydrobenzo[f][1,4]oxazepin- 5(2H)-one | 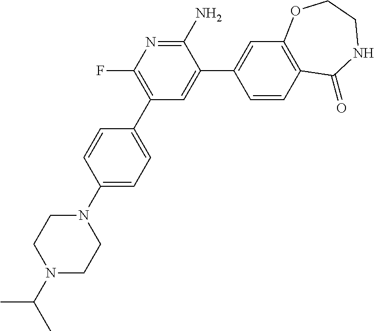 [SMILES: CC(C)N1CCN(c2ccc(-c3cc(-c4ccc5c(c4)OCCNC5=O)c(N)nc3F)cc2)CC1] |
| I-359 | 6-(2-amino-5-(4-(1,1- dioxidothiomorpholino)phenyl)- 6-fluoropyridin-3-yl)-8- fluoro-3,4- dihydroisoquinolin-1(2H)- one | 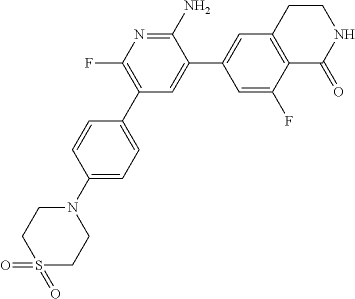 [SMILES: Nc1nc(F)c(-c2ccc(N3CCS(=O)(=O)CC3)cc2)cc1-c1cc(F)c2c(c1)CCNC2=O] |
| I-360 | 6-(2-amino-6-fluoro-5-(4- (morpholinomethyl)phenyl)py- ridin-3-yl)-8-fluoro-3,4- dihydroisoquinolin-1(2H)- one | 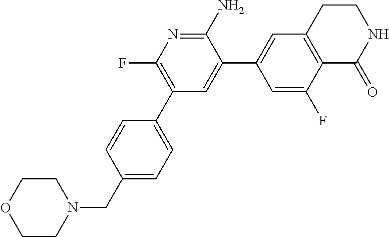 [SMILES: Nc1nc(F)c(-c2ccc(CN3CCOCC3)cc2)cc1-c1cc(F)c2c(c1)CCNC2=O] |
| I-361 | 6-(2-amino-6-fluoro-5-(3- fluoro-4- morpholinophenyl)pyridin-3- yl)-8-fluoro-3,4- dihydroisoquinolin-1(2H)- one | 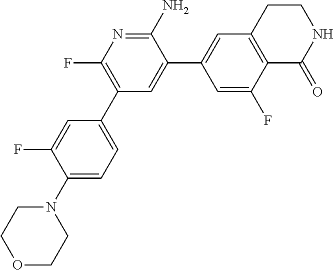 [SMILES: Nc1nc(F)c(-c2ccc(N3CCOCC3)c(F)c2)cc1-c1cc(F)c2c(c1)CCNC2=O] |
| I-362 | 7-(2-amino-6-fluoro-5-(4-(4- isopropylpiperazin-1- yl)phenyl)pyridin-3- yl)quinazolin-4(3H)-one | 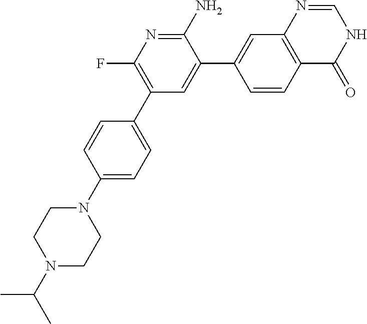 [SMILES: CC(C)N1CCN(c2ccc(-c3cc(-c4ccc5c(=O)[nH]cnc5c4)c(N)nc3F)cc2)CC1] |
| I-363 | 6-(2-amino-6-fluoro-5-(4-(4- isopropylpiperazin-1- yl)phenyl)pyridin-3- yl)phthalazin-1(2H)-one | 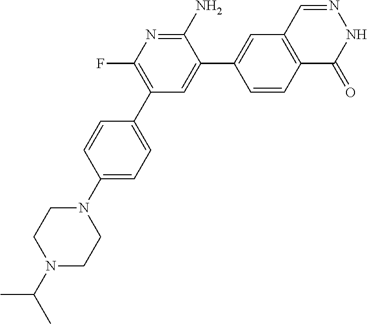 [SMILES: CC(C)N1CCN(c2ccc(-c3cc(-c4ccc5c(=O)[nH]ncc5c4)c(N)nc3F)cc2)CC1] |
| I-364 | 6-(2-amino-6-fluoro-5-(4-(4- isopropylpiperazin-1- yl)phenyl)pyridin-3- yl)isoquinolin-1(2H)-one | 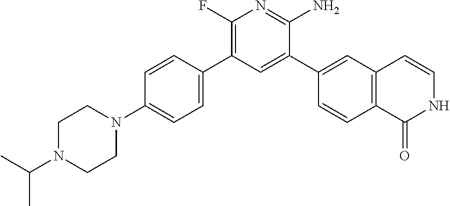 [SMILES: CC(C)N1CCN(c2ccc(-c3cc(-c4ccc5c(=O)[nH]ccc5c4)c(N)nc3F)cc2)CC1] |
| I-365 | 6′-(2-amino-6-fluoro-5-(4-(4- isopropylpiperazin-1- yl)phenyl)pyridin-3-yl)-2′,3′- dihydro-1′H- spiro[cyclopropane-1,4′- isoquinolin]-1′-one | 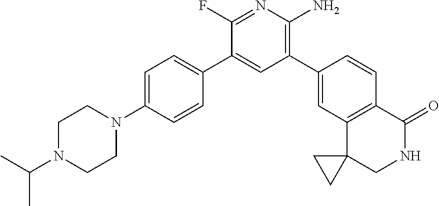 [SMILES: CC(C)N1CCN(c2ccc(-c3cc(-c4ccc5c(c4)C4(CC4)CNC5=O)c(N)nc3F)cc2)CC1] |
| I-366 | 6-(2-amino-6-fluoro-5-(4-(4- isopropylpiperazin-1- yl)phenyl)pyridin-3-yl)-2,3- dihydro-1H- spiro[isoquinoline-4,3′- oxetan]-1-one | 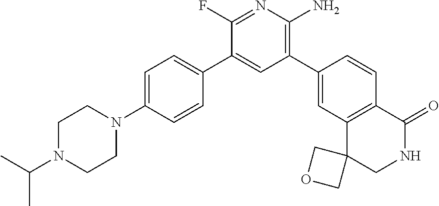 [SMILES: CC(C)N1CCN(c2ccc(-c3cc(-c4ccc5c(c4)C4(CNC5=O)COC4)c(N)nc3F)cc2)CC1] |
| I-367 | (S)-6-(2-amino-6-fluoro-5-(3- (pyrrolidin-2-yl)-4- (tetrahydro-2H-pyran-4- yl)phenyl)pyridin-3-yl)-3,4- dihydroisoquinolin-1(2H)- one | 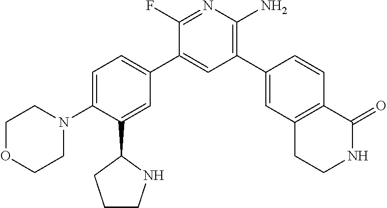 [SMILES: Nc1nc(F)c(-c2ccc(N3CCOCC3)c([C@@H]3CCCN3)c2)cc1-c1ccc2c(c1)CCNC2=O] |
| I-368 | (S)-6-(2-amino-6-fluoro-5-(3- (pyrrolidin-2-yl)-4- (tetrahydro-2H-pyran-4- yl)phenyl)pyridin-3-yl)-7- fluoro-3,4- dihydroisoquinolin-1(2H)- one | 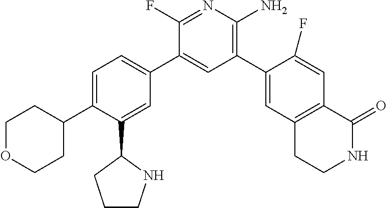 [SMILES: Nc1nc(F)c(-c2ccc(C3CCOCC3)c([C@@H]3CCCN3)c2)cc1-c1cc2c(cc1F)C(=O)NCC2] |
| I-369 | (S)-6-(2-amino-6-fluoro-5-(3- (pyrrolidin-2-yl)-4- (tetrahydro-2H-pyran-4- y)phenyl)pyridin-3-yl)-8- fluoro-3,4- dihydroisoquinolin-1(2H)- one | 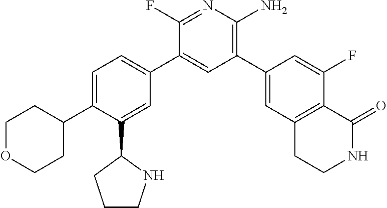 [SMILES: Nc1nc(F)c(-c2ccc(C3CCOCC3)c([C@@H]3CCCN3)c2)cc1-c1cc(F)c2c(c1)CCNC2=O] |
| I-370 | (S)-6-(2-amino-6-fluoro-5-(3- (pyrrolidin-2-yl)-4- (tetrahydro-2H-pyran-4- yl)phenyl)pyridin-3- yl)isoquinolin-1(2H)-one | 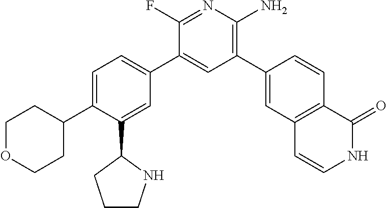 [SMILES: Nc1nc(F)c(-c2ccc(C3CCOCC3)c([C@@H]3CCCN3)c2)cc1-c1ccc2c(=O)[nH]ccc2c1] |
| I-372 | 6-(2-amino-6-fluoro-5-(4-(4- isopropylpiperazin-1- yl)phenyl)pyridin-3-yl)-8- fluoroisoquinolin-1(2H)-one | 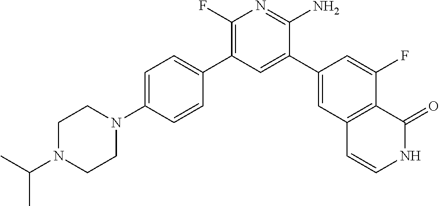 [SMILES: CC(C)N1CCN(c2ccc(-c3cc(-c4cc(F)c5c(=O)[nH]ccc5c4)c(N)nc3F)cc2)CC1] |
| I-373 | 6-(2-amino-6-fluoro-5-(4-(4- isopropylpiperazin-1- yl)phenyl)pyridin-3-yl)-7- fluoroisoquinolin-1(2H)-one | 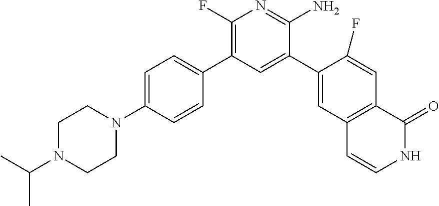 [SMILES: CC(C)N1CCN(c2ccc(-c3cc(-c4cc5cc[nH]c(=O)c5cc4F)c(N)nc3F)cc2)CC1] |
| I-374 | 6-(2-amino-5-(4-(4- (cyclopropylmethyl)piperazin- 1-yl)phenyl)-6-fluoropyridin- 3-yl)isoquinolin-1(2H)-one | 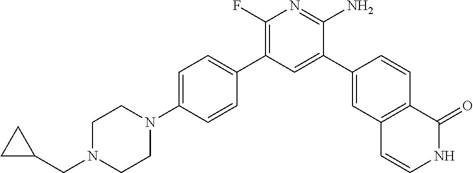 [SMILES: Nc1nc(F)c(-c2ccc(N3CCN(CC4CC4)CC3)cc2)cc1-c1ccc2c(=O)[nH]ccc2c1] |
| I-375 | 6-(2-amino-5-(4-(4- (cyclopropylmethyl)piperazin- 1-yl)phenyl)-6-fluoropyridin- 3-yl)-8-fluoroisoquinolin- 1(2H)-one | 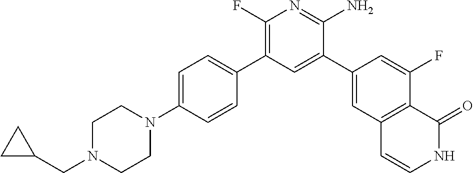 [SMILES: Nc1nc(F)c(-c2ccc(N3CCN(CC4CC4)CC3)cc2)cc1-c1cc(F)c2c(=O)[nH]ccc2c1] |
| I-376 | 6-(2-amino-5-(4-(4- (cylcopropylmethyl)piperazin- 1-yl)phenyl)-6-fluoropyridin- 3-yl)-7-fluoroisoquinolin- 1(2H)-one | 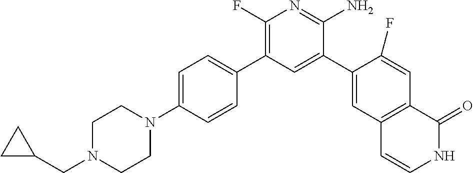 [SMILES: Nc1nc(F)c(-c2ccc(N3CCN(CC4CC4)CC3)cc2)cc1-c1cc2cc[nH]c(=O)c2cc1F] |
| I-377 | 6-(2-amino-6-fluoro-5-(4-(4- isopropylpiperazin-1- yl)phenyl)pyridin-3-yl)-3- methylisoquinolin-1(2H)-one | 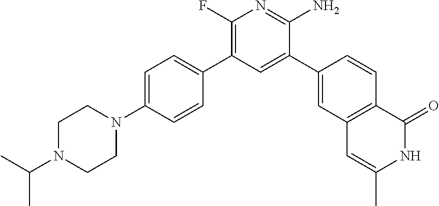 [SMILES: Cc1cc2cc(-c3cc(-c4ccc(N5CCN(C(C)C)CC5)cc4)c(F)nc3N)ccc2c(=O)[nH]1] |
| I-378 | 6-(2-amino-6-fluoro-5-(4-(4- isopropylpiperazin-1- yl)phenyl)pyridin-3-yl)-4- methylisoquinolin-1(2H)-one | 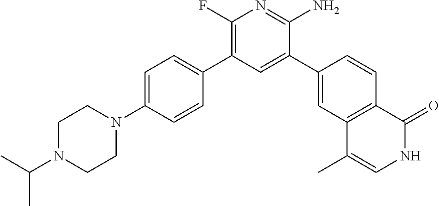 [SMILES: Cc1c[nH]c(=O)c2ccc(-c3cc(-c4ccc(N5CCN(C(C)C)CC5)cc4)c(F)nc3N)cc12] |
| I-379 | 6-(2-amino-6-fluoro-5-(4-(4- isopropylpiperazin-1- yl)phenyl)pyridin-3-yl)-4- fluoroisoquinolin-1(2H)-one | 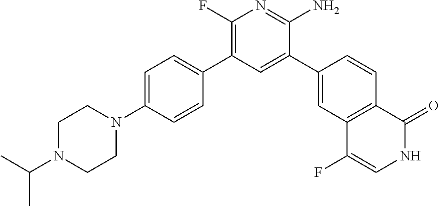 [SMILES: CC(C)N1CCN(c2ccc(-c3cc(-c4ccc5c(=O)[nH]cc(F)c5c4)c(N)nc3F)cc2)CC1] |
| I-380 | 6-(2-amino-6-fluoro-5-(4-(4- isopropylpiperazin-1- yl)phenyl)pyridin-3-yl)-8- fluoro-3-methylisoquinolin- 1(2H)-one | 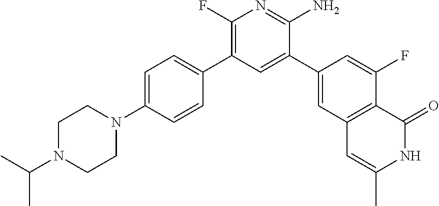 [SMILES: Cc1cc2cc(-c3cc(-c4ccc(N5CCN(C(C)C)CC5)cc4)c(F)nc3N)cc(F)c2c(=O)[nH]1] |
| I-381 | 6-(2-amino-6-fluoro-5-(4-(4- isopropylpiperazin-1- yl)phenyl)pyridin-3-yl)-4- fluoro-3-methylisoquinolin- 1(2H)-one | 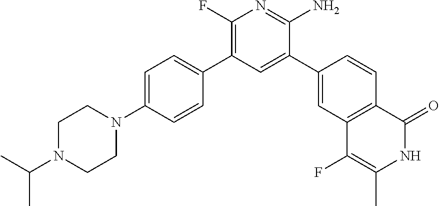 [SMILES: Cc1[nH]c(=O)c2ccc(-c3cc(-c4ccc(N5CCN(C(C)C)CC5)cc4)c(F)nc3N)cc2c1F] |
| I-384 | 6-(2-amino-5-(4-(4-(2- cyclopropylethyl)piperazin-1- yl)phenyl)-6-fluoropyridin-3- yl)-8-fluoro-3,4- dihydroisoquinolin-1(2H)- one | 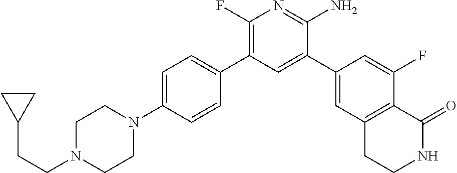 [SMILES: Nc1nc(F)c(-c2ccc(N3CCN(CCC4CC4)CC3)cc2)cc1-c1cc(F)c2c(c1)CCNC2=O] |
| I-385 | 6-(2-amino-5-(4-(4- (cyclopropylmethyl)piperazin- 1-yl)-3-fluorophenyl)-6- fluoropyridin-3-yl)-8-fluoro- 3,4-dihydroisoquinolin- 1(2H)-one | 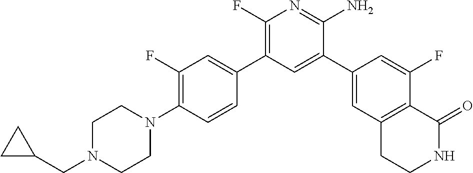 [SMILES: Nc1nc(F)c(-c2ccc(N3CCN(CC4CC4)CC3)c(F)c2)cc1-c1cc(F)c2c(c1)CCNC2=O] |
| I-386 | 6-(2-amino-6-fluoro-5-(3- fluoro-4-(4- isopropylpiperazin-1- yl)phenyl)pyridin-3-yl)-8- fluoro-3,4- dihydroisoquinolin-1(2H)- one | 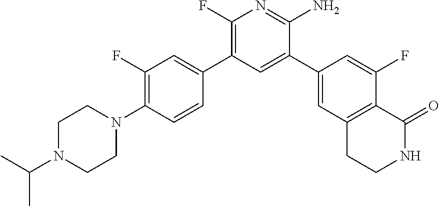 [SMILES: CC(C)N1CCN(c2ccc(-c3cc(-c4cc(F)c5c(c4)CCNC5=O)c(N)nc3F)cc2F)CC1] |
| I-387 | 6-(2-amino-5-(4-(4-(2- cyclopropylethyl)piperazin-1- yl)phenyl)-6-fluoropyridin-3- yl)-8-fluoroisoquinolin-1(2H)- one | 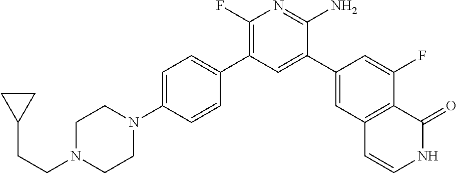 [SMILES: Nc1nc(F)c(-c2ccc(N3CCN(CCC4CC4)CC3)cc2)cc1-c1cc(F)c2c(=O)[nH]ccc2c1] |
| I-388 | 6-(2-amino-5-(4-(4-(2- cyclopropylethyl)piperazin-1- yl)phenyl)-6-fluoropyridin-3- yl)-7-fluoroisoquinolin-1(2H)- one | 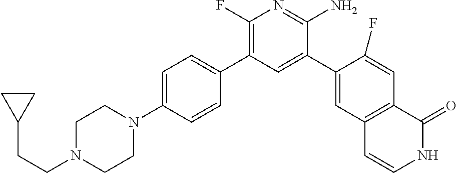 [SMILES: Nc1nc(F)c(-c2ccc(N3CCN(CCC4CC4)CC3)cc2)cc1-c1cc2cc[nH]c(=O)c2cc1F] |
| I-389 | 6-(2-amino-5-(4-(4-(2- cyclopropylethyl)piperazin-1- yl)phenyl)-6-fluoropyridin-3- yl)isoquinolin-1(2H)-one | 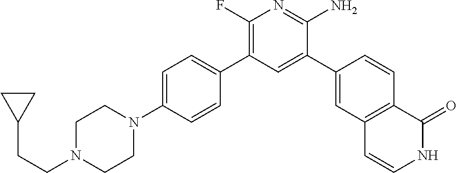 [SMILES: Nc1nc(F)c(-c2ccc(N3CCN(CCC4CC4)CC3)cc2)cc1-c1ccc2c(=O)[nH]ccc2c1] |
| I-390 | (R)-6-(2-amino-5-(4-(4-(1- cyclopropylethyl)piperazin-1- yl)phenyl)-6-fluoropyridin-3- yl)-8-fluoro-3,4- dihydroisoquinolin-1(2H)- one | 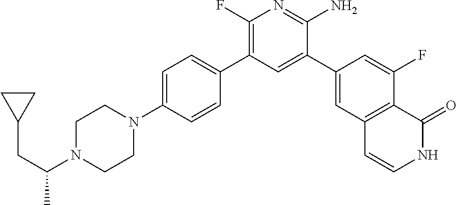 [SMILES: C[C@H](CC1CC1)N1CCN(c2ccc(-c3cc(-c4cc(F)c5c(=O)[nH]ccc5c4)c(N)nc3F)cc2)CC1] |
| I-391 | (R)-6-(2-amino-5-(4-(4-(1- cyclopropylethyl)piperazin-1- yl)phenyl)-6-fluoropyridin-3- yl)-8-fluoroisoquinolin-1(2H)- one | 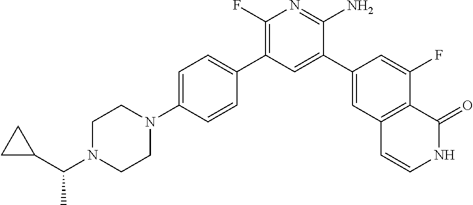 [SMILES: C[C@H](C1CC1)N1CCN(c2ccc(-c3cc(-c4cc(F)c5c(=O)[nH]ccc5c4)c(N)nc3F)cc2)CC1] |
| I-392 | (S)-6-(2-amino-5-(4-(4-(1- cyclopropylethyl)piperazin-1- yl)phenyl)-6-fluoropyridin-3- yl)-8-fluoro-3,4- dihydroisoquinolin-1(2H)- one | 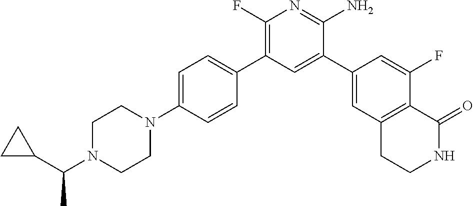 [SMILES: C[C@@H](C1CC1)N1CCN(c2ccc(-c3cc(-c4cc(F)c5c(c4)CCNC5=O)c(N)nc3F)cc2)CC1] |
| I-393 | (S)-6-(2-amino-5-(4-(4-(1- cyclopropylethyl)piperazin-1- y)phenyl)-6-fluoropyridin-3- yl)-8-fluoroisoquinolin-1(2H)- one | 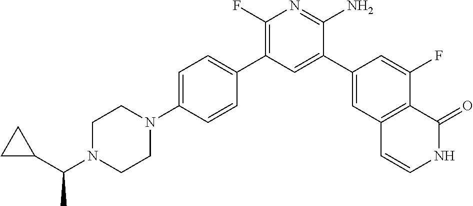 [SMILES: C[C@@H](C1CC1)N1CCN(c2ccc(-c3cc(-c4cc(F)c5c(=O)[nH]ccc5c4)c(N)nc3F)cc2)CC1] |
| I-394 | 6-(2-amino-5-(4-(4- ethylpiperazin-1-yl)phenyl)- 6-fluoropyridin-3-yl)-8- fluoroisoquinolin-1(2H)-one | 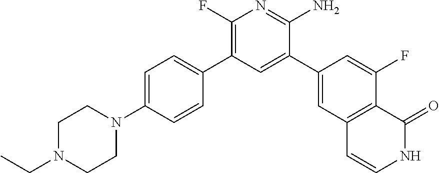 [SMILES: CCN1CCN(c2ccc(-c3cc(-c4cc(F)c5c(=O)[nH]ccc5c4)c(N)nc3F)cc2)CC1] |
| I-396 | 6-(2-amino-6-fluoro-5-(4- morpholinophenyl)pyridin-3- yl)-8-fluoroisoquinolin-1(2H)- one | 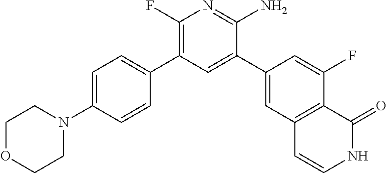 [SMILES: Nc1nc(F)c(-c2ccc(N3CCOCC3)cc2)cc1-c1cc(F)c2c(=O)[nH]ccc2c1] |
| I-397 | (S)-6-(2-amio-6-fluoro-5-(4- (2- isopropylmorpholino)phenyl) pyridin-3-yl)-3,4-dihydro-2,7- naphthyridin-1(2H)-one | 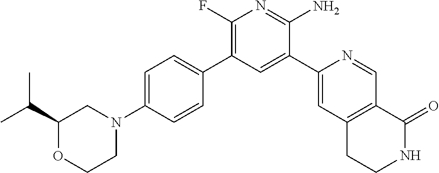 [SMILES: CC(C)[C@H]1CN(c2ccc(-c3cc(-c4cc5c(cn4)C(=O)NCC5)c(N)nc3F)cc2)CCO1] |
| I-398 | 6-(2-amino-6-fluoro-5-(4-(4- isopropylpiperazin-1- yl)phenyl)pyridin-3-yl)-3,4- dihydro-2,7-naphthyridin- 1(2H)-one | 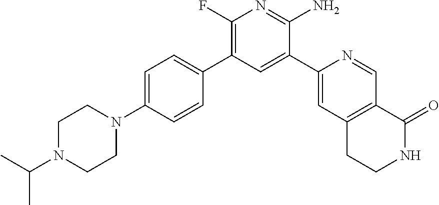 [SMILES: CC(C)N1CCN(c2ccc(-c3cc(-c4cc5c(cn4)C(=O)NCC5)c(N)nc3F)cc2)CC1] |
| I-399 | 3-(2-amino-6-fluoro-5-(4- morpholinophenyl)pyridin-3- yl)-1,7-naphthyridin-8(7H)- one | 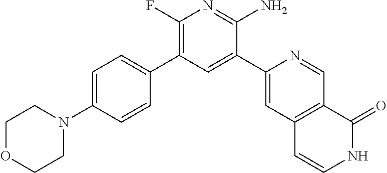 [SMILES: Nc1nc(F)c(-c2ccc(N3CCOCC3)cc2)cc1-c1cc2cc[nH]c(=O)c2cn1] |
| I-400 | 6-(2-amino-5-(4-(4- (cyclopropylmethyl)piperazin- 1-yl)phenyl)-6-fluoropyridin- 3-yl)-3,4-dihydro-2,7- naphthyridin-1(2H)-one | 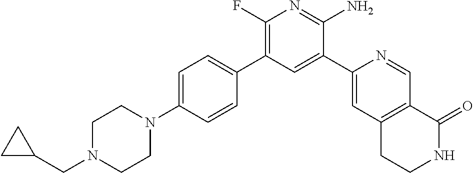 [SMILES: Nc1nc(F)c(-c2ccc(N3CCN(CC4CC4)CC3)cc2)cc1-c1cc2c(cn1)C(=O)NCC2] |
| I-401 | 6-(2-amino-6-fluoro-5-(4-(4- isopropylpiperazin-1- yl)phenyl)pyridin-3-yl)-2,7- naphthyridin-1(2H)-one | 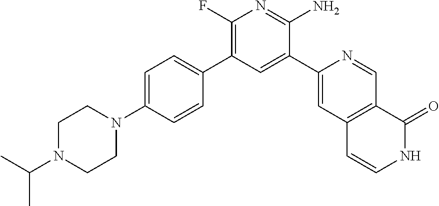 [SMILES: CC(C)N1CCN(c2ccc(-c3cc(-c4cc5cc[nH]c(=O)c5cn4)c(N)nc3F)cc2)CC1] |
| I-402 | 3-(2-amino-6-fluoro-5-(4-(4- isopropylpiperazin-1- yl)phenyl)pyridin-3-yl)-1,7- naphthyridin-8(7H)-one | 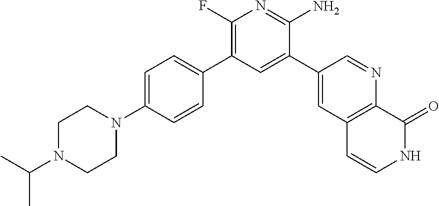 [SMILES: CC(C)N1CCN(c2ccc(-c3cc(-c4cnc5c(=O)[nH]ccc5c4)c(N)nc3F)cc2)CC1] |
| I-405 | 7-(2-amino-6-fluoro-5-(4-(4- isopropylpiperazin-1- yl)phenyl)pyridin-3-yl)-5- fluoroquinazolin-4(3H)-one | 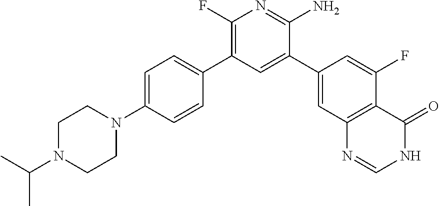 [SMILES: CC(C)N1CCN(c2ccc(-c3cc(-c4cc(F)c5c(=O)[nH]cnc5c4)c(N)nc3F)cc2)CC1] |
| I-406 | 7-(2-amino-6-fluoro-5-(4-(4- isopropylpiperazin-1- yl)phenyl)pyridin-3-yl)-6- fluoroquinazolin-4(3H)-one | 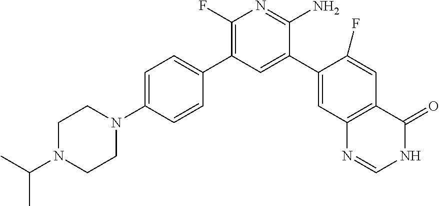 [SMILES: CC(C)N1CCN(c2ccc(-c3cc(-c4cc5nc[nH]c(=O)c5cc4F)c(N)nc3F)cc2)CC1] |
| I-407 | 7-(2-amino-5-(4-(4- (cyclopropylmethyl)piperazin- 1-yl)phenyl)-6-fluoropyridin- 3-yl)-5-fluoroquinazolin- 4(3H)-one | 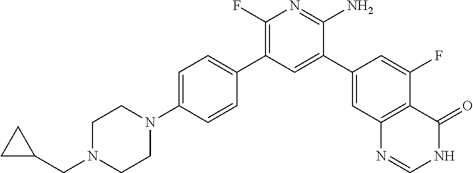 [SMILES: Nc1nc(F)c(-c2ccc(N3CCN(CC4CC4)CC3)cc2)cc1-c1cc(F)c2c(=O)[nH]cnc2c1] |
| I-408 | 7-(2-amino-5-(4-(4- (cyclopropylmethyl)piperazin- 1-yl)phenyl)-6-fluoropyridin- 3-yl)-6-fluoroquinazolin- 4(3H)-one | 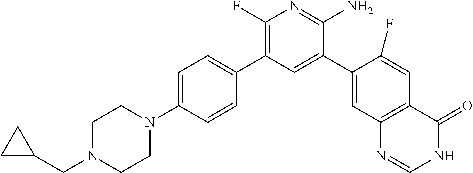 [SMILES: Nc1nc(F)c(-c2ccc(N3CCN(CC4CC4)CC3)cc2)cc1-c1cc2nc[nH]c(=O)c2cc1F] |
| I-409 | 7-(2-amino-6-fluoro-5-(4-(4- isopropylpiperazin-1- yl)phenyl)pyridin-3-yl)-2- methylquinazolin-4(3H)-one | 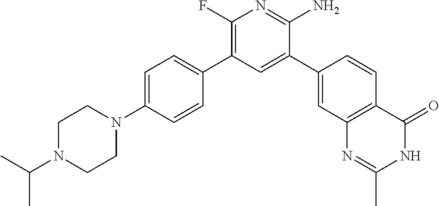 [SMILES: Cc1nc2cc(-c3cc(-c4ccc(N5CCN(C(C)C)CC5)cc4)c(F)nc3N)ccc2c(=O)[nH]1] |
| I-410 | 7-(2-amino-6-fluoro-5-(4-(4- isopropylpiperazin-1- yl)phenyl)pyridin-3-yl)-2- ((methylamino)methyl)quina- zolin-4(3H)-one | 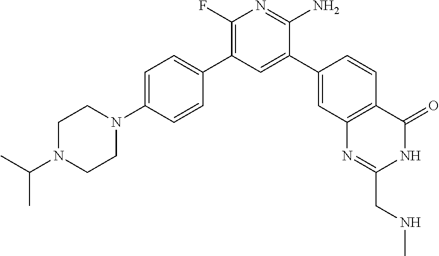 [SMILES: CNCc1nc2cc(-c3cc(-c4ccc(N5CCN(C(C)C)CC5)cc4)c(F)nc3N)ccc2c(=O)[nH]1] |
| I-411 | 7-(2-amino-5-(4-(4- (cyclopropylmethyl)piperazin- 1-yl)phenyl)-6-fluoropyridin- 3-yl)-2-methylquinazolin- 4(3H)-one | 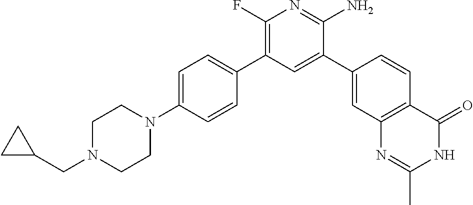 [SMILES: Cc1nc2cc(-c3cc(-c4ccc(N5CCN(CC6CC6)CC5)cc4)c(F)nc3N)ccc2c(=O)[nH]1] |
| I-412 | 7-(2-amino-6-fluoro-5-(4-(4- isopropylpiperazin-1- yl)phenyl)pyridin-3-yl)-2- cyclopropylquinazolin-4(3H)- one | 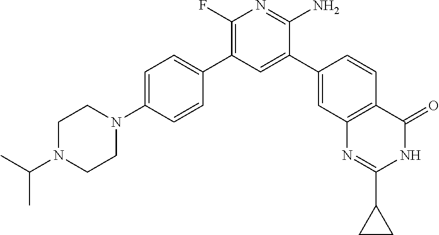 [SMILES: CC(C)N1CCN(c2ccc(-c3cc(-c4ccc5c(=O)[nH]c(C6CC6)nc5c4)c(N)nc3F)cc2)CC1] |
| I-413 | 7-(2-amino-6-fluoro-5-(4-(4- isopropylpiperazin-1- yl)phenyl)pyridin-3-yl)-2- cyclobutylquinazolin-4(3H)- one | 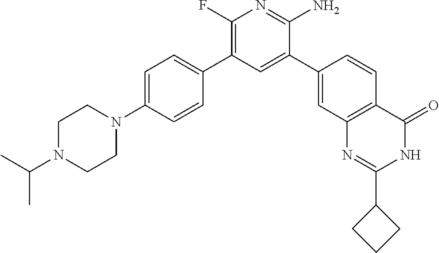 [SMILES: CC(C)N1CCN(c2ccc(-c3cc(-c4ccc5c(=O)[nH]c(C6CCC6)nc5c4)c(N)nc3F)cc2)CC1] |
| I-414 | 7-(2-amino-6-fluoro-5-(4-(4- isopropylpiperazin-1- yl)phenyl)pyridin-3-yl)-5- fluoro-2-methylquinazolin- 4(3H)-one | 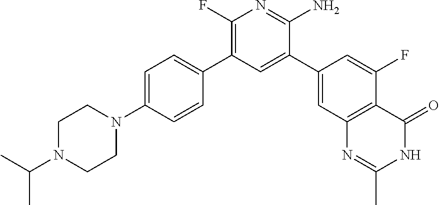 [SMILES: Cc1nc2cc(-c3cc(-c4ccc(N5CCN(C(C)C)CC5)cc4)c(F)nc3N)cc(F)c2c(=O)[nH]1] |
| I-415 | 7-(2-amino-6-fluoro-5-(4-(4- isopropylpiperazin-1- yl)phenyl)pyridin-3-l)-2- methylpyrido[4,3- d]pyrimidin-4(3H)-one | 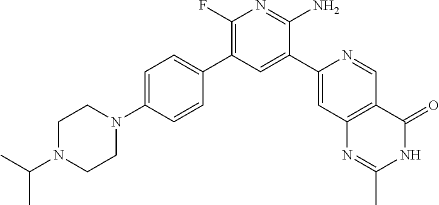 [SMILES: Cc1nc2cc(-c3cc(-c4ccc(N5CCN(C(C)C)CC5)cc4)c(F)nc3N)ncc2c(=O)[nH]1] |
| I-416 | 7-(2-amino-5-(4-(4- (cyclopropylmethyl)piperazin- 1-yl)phenyl)-6-fluoropyridin- 3-yl)-5-fluoro-2- methylquinazolin-4(3H)-one | 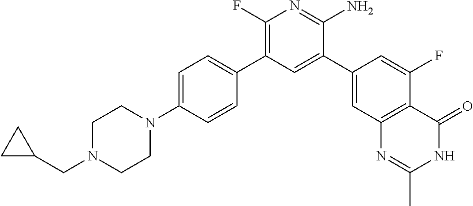 [SMILES: Cc1nc2cc(-c3cc(-c4ccc(N5CCN(CC6CC6)CC5)cc4)c(F)nc3N)cc(F)c2c(=O)[nH]1] |
| I-417 | 7-(2-amino-5-(4-(4-(2- cyclopropylethyl)piperazin-1- yl)phenyl)-6-fluoropyridin-3- yl)-5-fluoro-2- methylquinazolin-4(3H)-one | 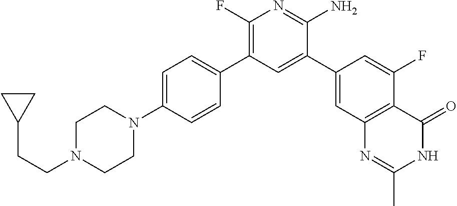 [SMILES: Cc1nc2cc(-c3cc(-c4ccc(N5CCN(CCC6CC6)CC5)cc4)c(F)nc3N)cc(F)c2c(=O)[nH]1] |
| I-418 | 7-(2-amino-6-fluoro-5-(4-(4- isopropylpiperazin-1- yl)phenyl)pyridin-3-yl)-2- chloroquinazolin-4(3H)-one | 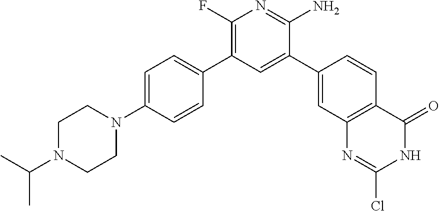 [SMILES: CC(C)N1CCN(c2ccc(-c3cc(-c4ccc5c(=O)[nH]c(Cl)nc5c4)c(N)nc3F)cc2)CC1] |
| I-419 | 7-(2-amino-6-fluoro-5-(4-(4- isopropylpiperazin-1- yl)phenyl)pyridin-3-yl)-2- methoxyquinazolin-4(3H)- one | 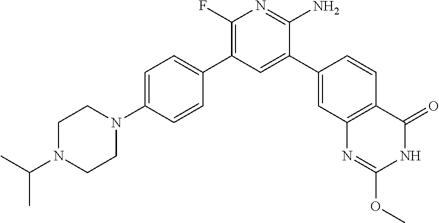 [SMILES: COc1nc2cc(-c3cc(-c4ccc(N5CCN(C(C)C)CC5)cc4)c(F)nc3N)ccc2c(=O)[nH]1] |
| I-420 | 7-(2-amino-6-fluoro-5-(4-(4- isopropylpiperazin-1- yl)phenyl)pyridin-3-yl)-2- (dimethylamino)quinazolin- 4(3H)-one | 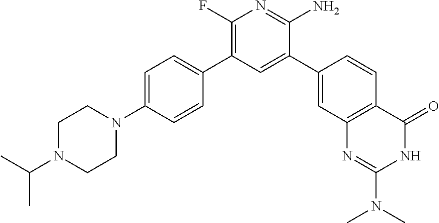 [SMILES: CC(C)N1CCN(c2ccc(-c3cc(-c4ccc5c(=O)[nH]c(N(C)C)nc5c4)c(N)nc3F)cc2)CC1] |
| I-421 | 7-(2-amino-6-fluoro-5-(4-(4- isopropylpiperazin-1- yl)phenyl)pyridin-3-yl)-2- (methylamino)quinazolin- 4(3H)-one | 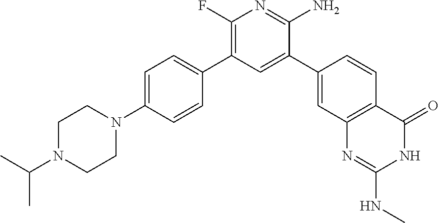 [SMILES: CNc1nc2cc(-c3cc(-c4ccc(N5CCN(C(C)C)CC5)cc4)c(F)nc3N)ccc2c(=O)[nH]1] |
| I-422 | 2-amino-7-(2-amino-6- fluoro-5-(4-(4- isopropylpiperazin-1- yl)phenyl)pyridin-3- yl)quinazolin-4(3H)-one | 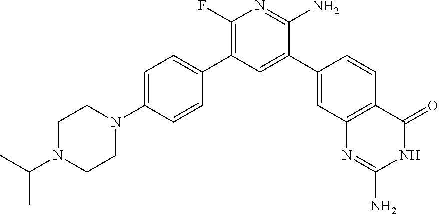 [SMILES: CC(C)N1CCN(c2ccc(-c3cc(-c4ccc5c(=O)[nH]c(N)nc5c4)c(N)nc3F)cc2)CC1] |
| I-424 | 7-(2-amino-6-fluoro-5-(4-(4- isopropylpiperazin-1- yl)phenyl)pyridin-3-yl)-2,3- dihydro-4H- benzo[e][1,3]thiazin-4-one 1,1-dioxide | 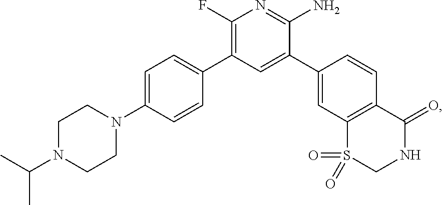 [SMILES: CC(C)N1CCN(c2ccc(-c3cc(-c4ccc5c(c4)S(=O)(=O)CNC5=O)c(N)nc3F)cc2)CC1] |
| | | and |
| I-425 | 6-(2-amino-6-fluoro-5-(4-(4- isopropylpiperazin-1- yl)phenyl)pyridin-3- yl)isoquinoline-1,3(2H,4H)- dione | 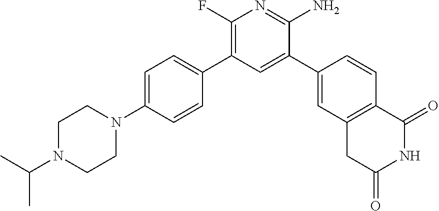 [SMILES: CC(C)N1CCN(c2ccc(-c3cc(-c4ccc5c(c4)CC(=O)NC5=O)c(N)nc3F)cc2)CC1] |
| I-426 | 6-(2-amino-5-(3- ((dimethylamino)methyl)-4- (tetrahydro-2H-pyran-4- yl)phenyl)-6-fluoropyridin-3- yl)-4-methylisoquinolin- 1(2H)-one | 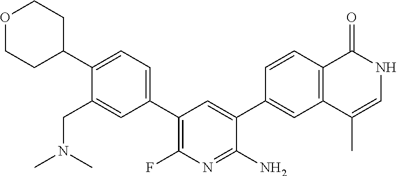 [SMILES: Cc1c[nH]c(=O)c2ccc(-c3cc(-c4ccc(C5CCOCC5)c(CN(C)C)c4)c(F)nc3N)cc12] |
| I-427 | 6-(6′-amino-6- ((dimethylamino)methyl)-2′- fluoro-5-morpholino-[2,3′- bipyridin]-5′-yl)-7-fluoro-3,4- dihydroisoquinolin-1(2H)- one | 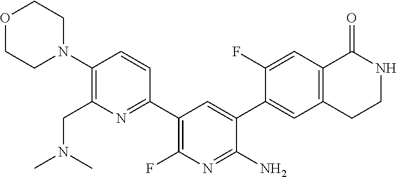 [SMILES: CN(C)Cc1nc(-c2cc(-c3cc4c(cc3F)C(=O)NCC4)c(N)nc2F)ccc1N1CCOCC1] |
| I-428 | 6-(2-amino-5-(3- ((ethyl(methyl)amino)methyl)- 4-(tetrahydro-2H-pyran-4- yl)phenyl)-6-fluoropyridin-3- yl)-3,4-dihydroisoquinolin- 1(2H)-one | 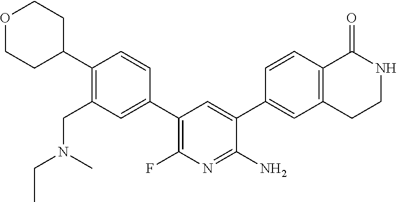 [SMILES: CCN(C)Cc1cc(-c2cc(-c3ccc4c(c3)CCNC4=O)c(N)nc2F)ccc1C1CCOCC1] |
| I-429 | 6-(2-amino-5-(3- ((dimethylamino)methyl)-4- (tetrahydro-2H-pyran-4- yl)phenyl)-6-fluoropyridin-3- yl)-7-fluoro-3,4- dihydroisoquinolin-1(2H)- one | 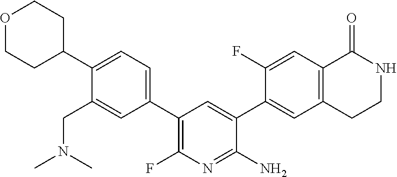 [SMILES: CN(C)Cc1cc(-c2cc(-c3cc4c(cc3F)C(=O)NCC4)c(N)nc2F)ccc1C1CCOCC1] |
| I-430 | 6-(2-amino-6-fluoro-5-(4-(2- methyl-2,7- diazaspiro[3.5]nonan-7- yl)phenyl)pyridin-3-yl)-3,4- dihydroisoquinolin-1(2H)- one | 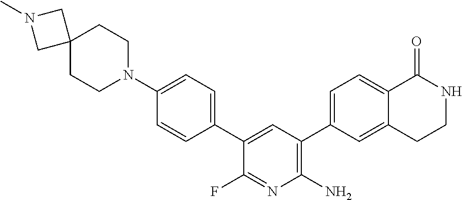 [SMILES: CN1CC2(CCN(c3ccc(-c4cc(-c5ccc6c(c5)CCNC6=O)c(N)nc4F)cc3)CC2)C1] |
| I-431 | 6-(2-amino-6-fluoro-5-(5- methyl-1,2,3,4- tetrahydroisoquinolin-7- yl)pyridin-3-yl)-3,4- dihydroisoquinolin-1(2H)- one | 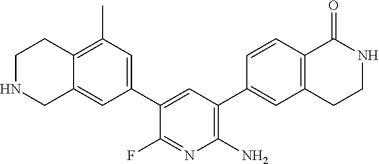 [SMILES: Cc1cc(-c2cc(-c3ccc4c(c3)CCNC4=O)c(N)nc2F)cc2c1CCNC2] |
| I-432 | 6-(2-amino-5-(3- ((dimethylamino)methyl)-4- morpholinophenyl)-6- fluoropyridin-3-yl)-3,4- dihydroisoquinolin-1(2H)- one | 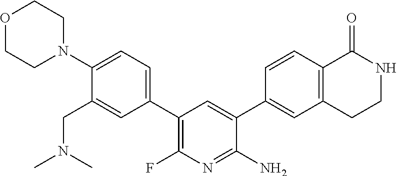 [SMILES: CN(C)Cc1cc(-c2cc(-c3ccc4c(c3)CCNC4=O)c(N)nc2F)ccc1N1CCOCC1] |
| I-433 | 6-(2-amino-5-(3- ((dimethylamino)methyl)-4- morpholinophenyl)-6- fluoropyridin-3-yl)-7-fluoro- 3,4-dihydroisoquinolin- 1(2H)-one | 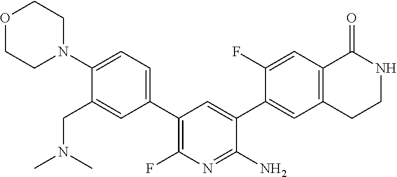 [SMILES: CN(C)Cc1cc(-c2cc(-c3cc4c(cc3F)C(=O)NCC4)c(N)nc2F)ccc1N1CCOCC1] |
| I-434 | 6-(2-amino-5-(2,5-dimethyl- 1,2,3,4- tetrahydroisoquinolin-7-yl)-6- fluropyridin-3-yl)-3,4- dihydroisoquinolin-1(2H)- one | 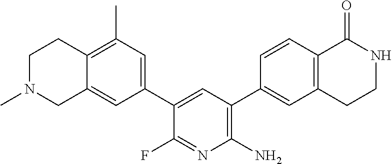 [SMILES: Cc1cc(-c2cc(-c3ccc4c(c3)CCNC4=O)c(N)nc2F)cc2c1CCN(C)C2] |
| I-435 | 7-(2-amino-6-fluoro-5-(4-(4- isopropylpiperazin-1- yl)phenyl)pyridin-3-yl)-2- (trifluoromethyl)quinazolin- 4(3H)-one | 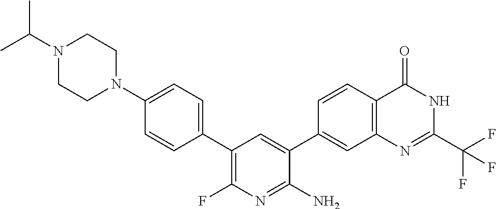 [SMILES: CC(C)N1CCN(c2ccc(-c3cc(-c4ccc5c(=O)[nH]c(C(F)(F)F)nc5c4)c(N)nc3F)cc2)CC1] |
| I-436 | 6-(2-amino-5-(3- ((dimethylamino)methyl)-4- (tetrahydro-2H-pyran-4- yl)phenyl)-6-fluoropyridin-3- yl)-8-fluoro-3,4- dihydroisoquinolin-1(2H)- one | 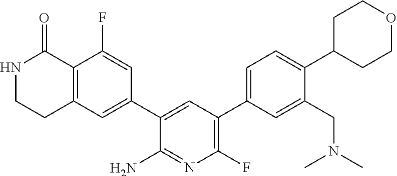 [SMILES: CN(C)Cc1cc(-c2cc(-c3cc(F)c4c(c3)CCNC4=O)c(N)nc2F)ccc1C1CCOCC1] |
| I-437 | 7-(2-amino-5-(3- ((dimethylamino)methyl)-4- (tetrahydro-2H-pyran-4- yl)phenyl)-6-fluoropyridin-3- yl)-2-methylquinazolin- 4(3H)-one | 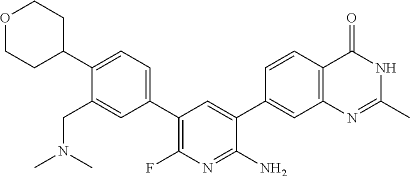 [SMILES: Cc1nc2cc(-c3cc(-c4ccc(C5CCOCC5)c(CN(C)C)c4)c(F)nc3N)ccc2c(=O)[nH]1] |
| I-438 | 6-(2-amino-6-fluoro-5-(6- methoxy-2-methyl-1,2,3,4- tetrahydroisoquinolin-7- yl)pyridin-3-yl)-3,4- dihydroisoquinolin-1(2H)- one | 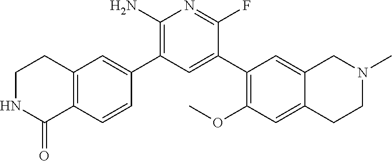 [SMILES: COc1cc2c(cc1-c1cc(-c3ccc4c(c3)CCNC4=O)c(N)nc1F)CN(C)CC2] |
| I-439 | 7-(2-amino-6-fluoro-5-(4-(4- isopropylpiperazin-1- yl)phenyl)pyridin-3-yl)-6- fluoro-2-methylquinazolin- 4(3H)-one | 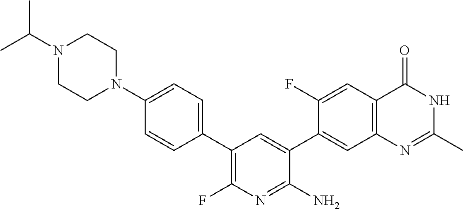 [SMILES: Cc1nc2cc(-c3cc(-c4ccc(N5CCN(C(C)C)CC5)cc4)c(F)nc3N)c(F)cc2c(=O)[nH]1] |
| I-440 | 6-(2-amino-5-(4-(4-(4,4- difluorobutyl)piperazin-1- yl)phenyl)-6-fluoropyridin-3- yl)-8-fluoro-3,4- dihydroisoquinolin-1(2H)- one | 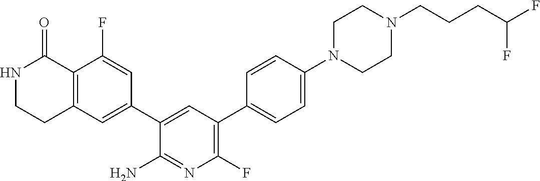 |
| I-441 | 6-(2-amino-5-(4-(4-(4,4- difluroobutyl)piperazin-1- yl)phenyl)-6-fluoropyridin-3- yl)-7-fluoro-3,4- dihydroisoquinoiln-1(2H)- one | 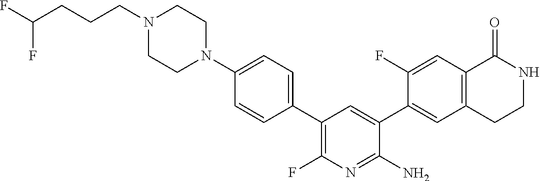 [SMILES: Nc1nc(F)c(-c2ccc(N3CCN(CCCC(F)F)CC3)cc2)cc1-c1cc2c(cc1F)C(=O)NCC2] |
| I-442 | tert-butyl 4-(4-(6-amino-2- fluoro-5-(1-oxo-1,2,3,4- tetrahydroisoquinolin-6- yl)pyridin-3-yl)-2- ((dimethylamino)methyl)phe- nyl)piperazine-1-carboxylate | 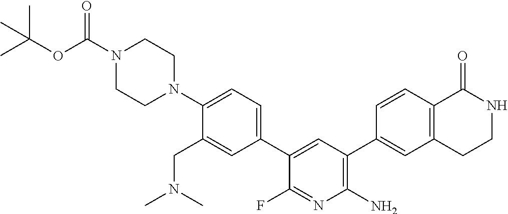 [SMILES: CN(C)Cc1cc(-c2cc(-c3ccc4c(c3)CCNC4=O)c(N)nc2F)ccc1N1CCN(C(=O)OC(C)(C)C)CC1] |
| I-443 | (S)-6-(2-amino-6-fluoro-5-(3- (1-methylpyrrolidin-2- yl)phenyl)pyridin-3-yl)-3,4- dihydroisoquinolin-1(2H)- one | 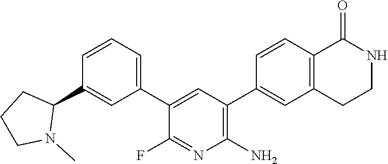 [SMILES: CN1CCC[C@H]1c1cccc(-c2cc(-c3ccc4c(c3)CCNC4=O)c(N)nc2F)c1] |
| I-444 | (S)-6-(2-amino-6-fluoro-5-(3- (1-methylpyrrolidin-2- yl)phenyl)pyridin-3-yl)-7- fluoro-3,4- dihydroisoquinolin-1(2H)- one | 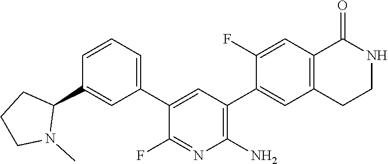 [SMILES: CN1CCC[C@H]1c1cccc(-c2cc(-c3cc4c(cc3F)C(=O)NCC4)c(N)nc2F)c1] |
| I-445 | 6-(2-amion-5-(4-(4-(4,4- difluorobutyl)piperazin-1-yl)- 3- ((dimethylamino)methyl)phe- nyl)-6-fluoropyridin-3-yl)-3,4- dihydroisoquinolin-1(2H)- one | 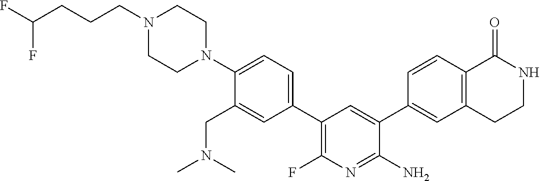 |
| I-446 | 6-(2-amino-5-(2-(4,4- difluorobutyl)-5-methyl- 1,2,3,4- tetrahydroisoquinolin-7-yl)-6- fluoropyridin-3-yl)-3,4- dihydroisoquinolin-1(2H)- one | 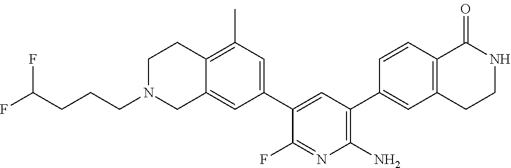 |
| I-447 | 6-(2-amino-5-(4-(4-(4,4- difluorobutyl)piperazin-1-yl)- 3- ((dimethylamino)methyl)phe- nyl)-6-fluoropyridin-3-yl)-7- fluoro-3,4- dihydroisoquinolin-1(2H)- one | 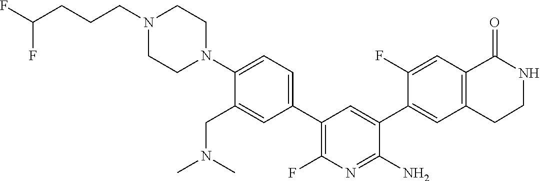 [SMILES: CN(C)Cc1cc(-c2cc(-c3cc4c(cc3F)C(=O)NCC4)c(N)nc2F)ccc1N1CCN(CCCC(F)F)CC1] |
| I-448 | 6-(2-amino-6-fluoro-5-(2-(2- methoxyethyl)-5-methyl- 1,2,3,4- tetrahydroisoquinolin-7- yl)pyridin-3-yl)-3,4- dihydroisoquinolin-1(2H)- one | 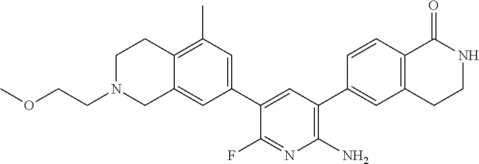 [SMILES: COCCN1CCc2c(C)cc(-c3cc(-c4ccc5c(c4)CCNC5=O)c(N)nc3F)cc2C1] |
| I-449 | 6-(2-amino-5-(4-(2- cyclobutyl-2,7- diazaspiro[3.5]nonan-7- yl)phenyl)-6-fluoropyridin-3- yl)-7-fluoro-3,4- dihydroisoquinolin-1(2H)- one | 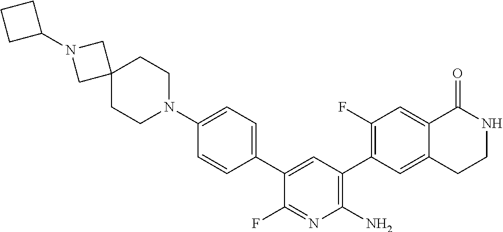 [SMILES: Nc1nc(F)c(-c2ccc(N3CCC4(CC3)CN(C3CCC3)C4)cc2)cc1-c1cc2c(cc1F)C(=O)NCC2] |
| I-450 | 6-(2-amino-6-fluoro-5-(4-(4- isopropylpiperazin-1- yl)phenyl)pyridin-3-yl)-4,4- difluoro-3-methoxy-3-methyl- 3,4-dihydroisoquinolin- 1(2H)-one | 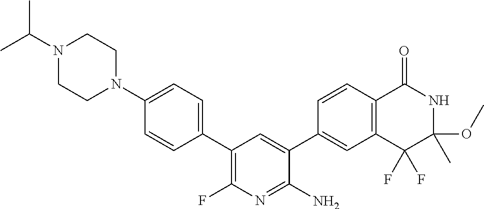 [SMILES: COC1(C)NC(=O)c2ccc(-c3cc(-c4ccc(N5CCN(C(C)C)CC5)cc4)c(F)nc3N)cc2C1(F)F] |
| I-451 | 6-(2-amino-5-(4-(4- cyclopentylpiperazin-1-yl)-3- ((dimethylamino)methyl)phe- nyl)-6-fluoropyridin-3-yl)-3,4- dihydroisoquinolin-1(2H)- one | 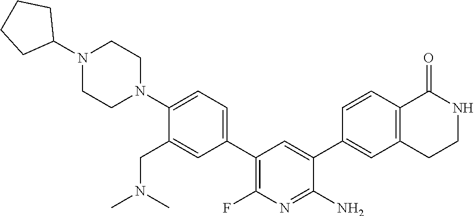 [SMILES: CN(C)Cc1cc(-c2cc(-c3ccc4c(c3)CCNC4=O)c(N)nc2F)ccc1N1CCN(C2CCCC2)CC1] |
| I-452 | 6-(2-amino-5-(4-(4- cyclopentylpiperazin-1-yl)-3- ((dimethylamino)methyl)phe- nyl)-6-fluoropyridin-3-yl)-7- fluoro-3,4- dihydroisoquinolin-1(2H)- one | 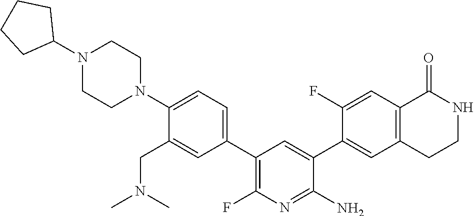 [SMILES: CN(C)Cc1cc(-c2cc(-c3cc4c(cc3F)C(=O)NCC4)c(N)nc2F)ccc1N1CCN(C2CCCC2)CC1] |
| I-453 | 6-(2-amino-5-(4-(4-(4,4- difluorobutyl)piperazin-1-yl)- 3- ((dimethylamino)methyl)phe- nyl)-6-fluoropyridin-3- yl)isoquinolin-1(2H)-one | 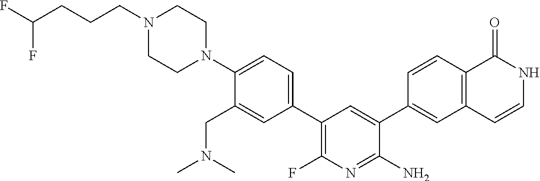 |
| I-454 | 6-(2-amino-5-(4-(4- (cyclopropylmethyl)piperazin- 1-yl)phenyl)-6-fluoropyridin- 3-yl)-8-fluoro-3- methylisoquinolin-1(2H)-one | 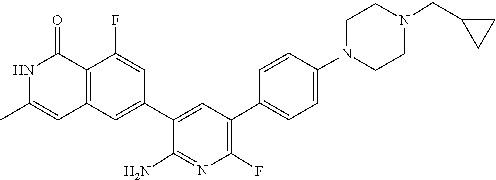 |
| I-455 | 6-(2-amino-5-(4-(4- (cyclopropylmethyl)piperazin- 1-yl)phenyl)-6-fluoropyridin- 3-yl)-4-fluoroisoquinolin- 1(2H)-one | 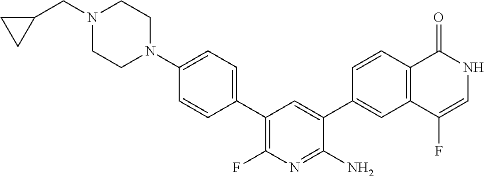 [SMILES: Nc1nc(F)c(-c2ccc(N3CCN(CC4CC4)CC3)cc2)cc1-c1ccc2c(=O)[nH]cc(F)c2c1] |
| I-456 | 6-(2-amino-5-(4-(4- (cyclopropylmethyl)piperazin- 1-yl)phenyl)-6-fluoropyridin- 3-yl)-4-fluoro-3- methylisoquinolin-1(2H)-one | 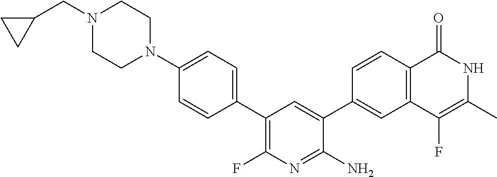 [SMILES: Cc1[nH]c(=O)c2ccc(-c3cc(-c4ccc(N5CCN(CC6CC6)CC5)cc4)c(F)nc3N)cc2c1F] |
| I-457 | 6-(2-amino-5-(4-(4- (cyclopropylmethyl)piperazin- 1-yl)phenyl)-6-fluoropyridin- 3-yl)-4-methylisoquinolin- 1(2H)-one | 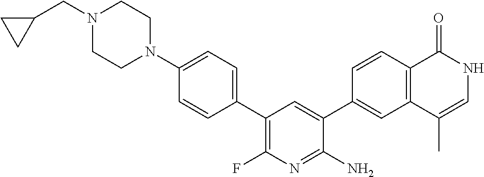 [SMILES: Cc1c[nH]c(=O)c2ccc(-c3cc(-c4ccc(N5CCN(CC6CC6)CC5)cc4)c(F)nc3N)cc12] |
| I-458 | 2-(2-amino-6-fluoro-5-(4-(4- isopropylpiperazin-1- yl)phenyl)pyridin-3-yl)-7,8- dihydropyrido[4,3- d]pyrimidin-5(6H)-one | 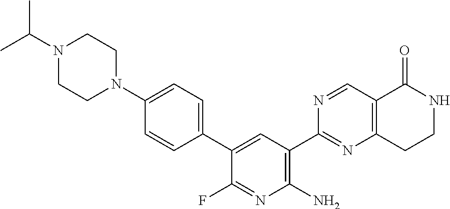 [SMILES: CC(C)N1CCN(c2ccc(-c3cc(-c4ncc5c(n4)CCNC5=O)c(N)nc3F)cc2)CC1] |
| I-459 | 6-(2-amino-5-(3- ((dimethylamino)methyl)-4- methoxyphenyl)-6- fluoropyridin-3-yl)-3,4- dihydroisoquinolin-1(2H)- one | 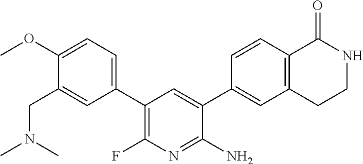 [SMILES: COc1ccc(-c2cc(-c3ccc4c(c3)CCNC4=O)c(N)nc2F)cc1CN(C)C] |
| I-460 | 6-(2-amino-5-(3- ((dimethylamino)methyl)-4- methoxyphenyl)-6- fluoropyridin-3-yl)-7-fluoro- 3,4-dihydroisoquinolin- 1(2H)-one | 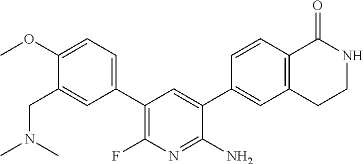 [SMILES: COc1ccc(-c2cc(-c3ccc4c(c3)CCNC4=O)c(N)nc2F)cc1CN(C)C] |
| I-461 | 6-(2-amino-5-(4-(4- cyclobutylpiperazin-1-yl)-3- ((dimethylamino)methyl)phe- nyl)-6-fluoropyridin-3-yl)-3,4- dihydroisoquinolin-1(2H)- one | 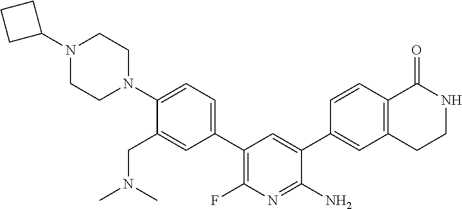 [SMILES: CN(C)Cc1cc(-c2cc(-c3ccc4c(c3)CCNC4=O)c(N)nc2F)ccc1N1CCN(C2CCC2)CC1] |
| I-462 | 6-(2-amino-5-(4-(4- cyclobutylpiperazin-1-yl)-3- ((dimethylamino)methyl)phe- nyl)-6-fluoropyridin-3-yl)-7- fluoro-3,4- dihydroisoquinolin-1(2H)- one | 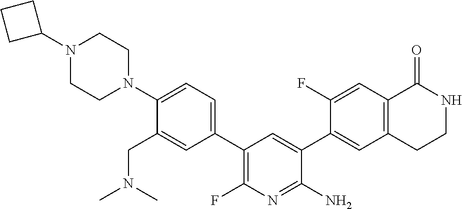 [SMILES: CN(C)Cc1cc(-c2cc(-c3cc4c(cc3F)C(=O)NCC4)c(N)nc2F)ccc1N1CCN(C2CCC2)CC1] |
| I-463 | 6-(2-amino-6-fluoro-5-(2- methyl-1,2,3,4- tetrahydroisoquinolin-5- yl)pyridin-3-yl)-3,4- dihydroisoquinolin-1(2H)- one | 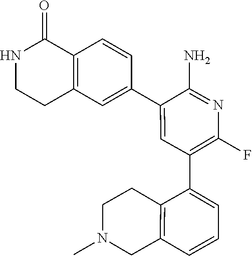 [SMILES: CN1CCc2c(cccc2-c2cc(-c3ccc4c(c3)CCNC4=O)c(N)nc2F)C1] |
| I-464 | 6-(2-amino-6-fluoro-5-(2- methyl-1,2,3,4- tetrahydroisoquinolin-5- yl)pyridin-3-yl)-7-fluoro-3,4- dihydroisoquinolin-1(2H)- one | 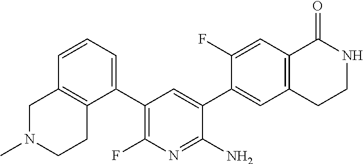 [SMILES: CN1CCc2c(cccc2-c2cc(-c3cc4c(cc3F)C(=O)NCC4)c(N)nc2F)C1] |
| I-465 | 3-amino-7-(2-amino-6- fluoro-5-(4-(4- isopropylpiperazin-1- yl)phenyl)pyridin-3-yl)-2- methylquinazolin-4(3H)-one | 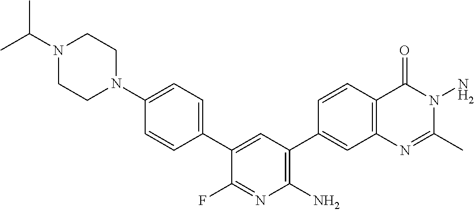 |
| I-466 | 6-(2-amino-6-fluoro-5-(4-(4- isopropylpiperazin-1- yl)phenyl)pyridin-3-yl)-4- chloroisoquinolin-1(2H)-one | 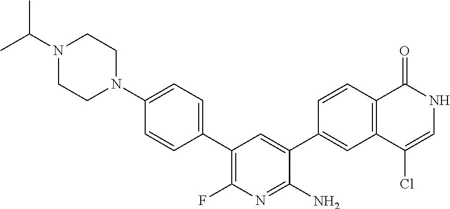 [SMILES: CC(C)N1CCN(c2ccc(-c3cc(-c4ccc5c(=O)[nH]cc(Cl)c5c4)c(N)nc3F)cc2)CC1] |
| I-467 | 7-(2-amino-5-(4-(4-(4,4- difluorobutyl)piperazin-1- yl)phenyl)-6-fluoropyridin-3- yl)-5-fluoro-2- methylquinazolin-4(3H)-one | 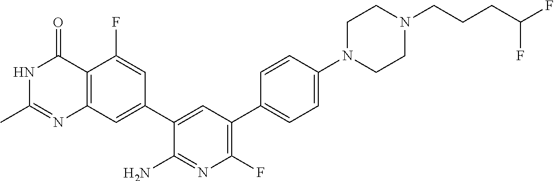 |
| I-468 | 7-(2-amino-6-fluoro-5-(4- ((1S,5R)-3-methyl-3- azabicyclo[3.1.0]hexan-1- yl)phenyl)pyridin-3-yl)-2- methylquinazolin-4(3H)-one | 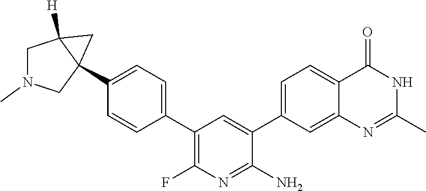 |
| I-469 | 7-(2-amino--fluoro-5-(4- ((1S,5R)-3-methyl-3- azabicyclo[3.1.0]hexan-1- yl)phenyl)pyridin-3-yl)-5- fluoro-2-methylquinazolin- 4(3H)-one | 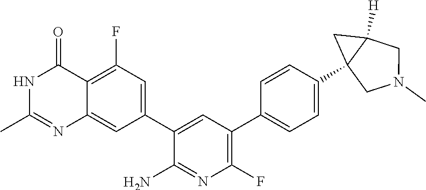 |
| I-470 | 6-(2-amino-6-fluoro-5-(4- ((1S,5R)-3-methyl-3- azabicyclo[3.1.0]hexan-1- yl)phenyl)pyridin-3-yl)-4- fluoroisoquinolin-1(2H)-one | 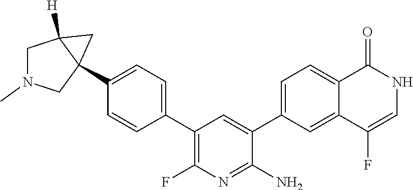 [SMILES: CN1C[C@@H]2C[C@]2(c2ccc(-c3cc(-c4ccc5c(=O)[nH]cc(F)c5c4)c(N)nc3F)cc2)C1] |
| I-471 | 6-(2-amino-6-fluoro-5-(4- ((1S,5R)-3-methyl-3- azabicyclo[3.1.0]hexan-1- yl)phenyl)pyridin-3-yl)-4- methylisoquinolin-1(2H)-one | 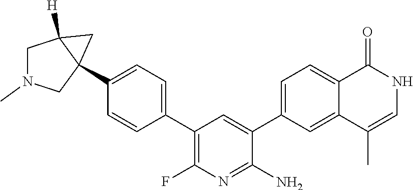 [SMILES: Cc1c[nH]c(=O)c2ccc(-c3cc(-c4ccc([C@]56C[C@H]5CN(C)C6)cc4)c(F)nc3N)cc12] |
| I-472 | 7-(2-amino-5-(4-(4- cyclobutylpiperazin-1- y)phenyl)-6-fluoropyridin-3- yl)-2-methylquinazolin- 4(3H)-one | 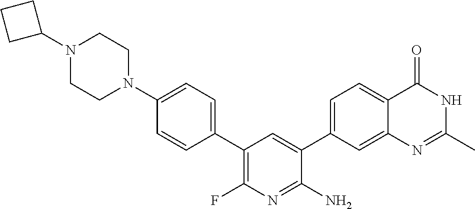 [SMILES: Cc1nc2cc(-c3cc(-c4ccc(N5CCN(C6CCC6)CC5)cc4)c(F)nc3N)ccc2c(=O)[nH]1] |
| I-473 | 7-(2-amino-5-(4-(4- cyclobutylpiperazin-1- yl)phenyl)-6-fluoropyridin-3- yl)-5-fluoro-2- methylquinazolin-4(3H)-one | 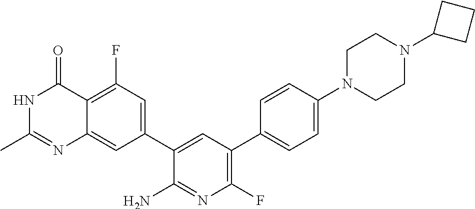 [SMILES: Cc1nc2cc(-c3cc(-c4ccc(N5CCN(C6CCC6)CC5)cc4)c(F)nc3N)cc(F)c2c(=O)[nH]1] |
| I-474 | 7-(2-amino-5-(3- ((dimethylamino)methyl)-4- morpholinophenyl)-6- fluoropyridin-3-yl)-2- methylquinazolin-4(3H)-one | 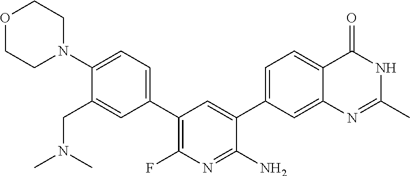 [SMILES: Cc1nc2cc(-c3cc(-c4ccc(N5CCOCC5)c(CN(C)C)c4)c(F)nc3N)ccc2c(=O)[nH]1] |
| I-475 | 7-(2-amino-5-(3- ((dimethylamino)methyl)-4- morpholinophenyl)-6- fluoropyridin-3-yl)-5-fluoro-2- methylquinazolin-4(3H)-one | 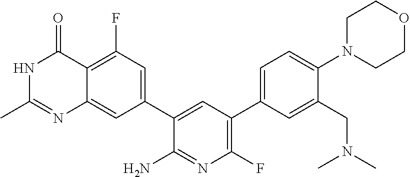 [SMILES: Cc1nc2cc(-c3cc(-c4ccc(N5CCOCC5)c(CN(C)C)c4)c(F)nc3N)cc(F)c2c(=O)[nH]1] |
| I-476 | 6-(2-amino-5-(4-(4-(3,3- difluoropropyl)piperazin-1- yl)phenyl)-6-fluoropyridin-3- yl)-3,4-dihydroisoquinolin- 1(2H)-one | 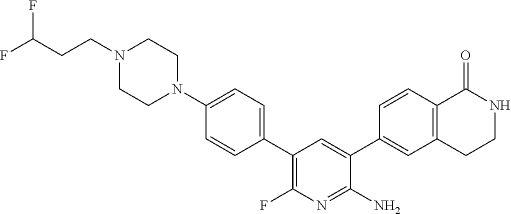 [SMILES: Nc1nc(F)c(-c2ccc(N3CCN(CCC(F)F)CC3)cc2)cc1-c1ccc2c(c1)CCNC2=O] |
| I-477 | 6-(2-amino-5-(4-(4-(3,3- difluoropropyl)piperazin-1- y)phenyl)-6-fluoropyridin-3- yl)-7-fluoro-3,4- dihydroisoquinolin-1(2H)- one | 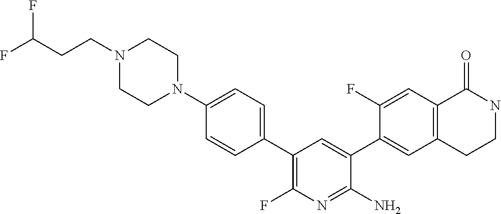 |
| I-478 | 6-(2-amino-5-(4-(3,6- dihydro-2H-pyran-4-yl)-2-(5- oxopyrrolidin-3-yl)phenyl)-6- fluoropyridin-3-yl)-7-fluoro- 3,4-dihydroisoquinolin- 1(2H)-one | 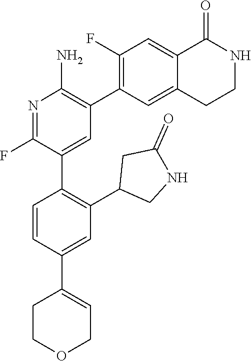 [SMILES: Nc1nc(F)c(-c2ccc(C3=CCOCC3)cc2C2CNC(=O)C2)cc1-c1cc2c(cc1F)C(=O)NCC2] |
| I-479 | 6-(2-amino-6-fluoro-5-(2-(5- oxopyrrolidin-3-yl)-4- (tetrahydro-2H-pyran-4- y)phenyl)pyridin-3-yl)-7- fluoro-3,4- dihydroisoquinolin-1(2H)- one | 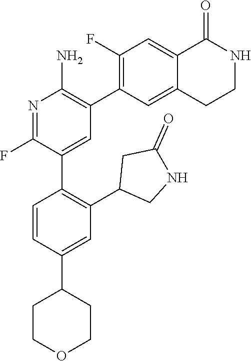 [SMILES: Nc1nc(F)c(-c2ccc(C3CCOCC3)cc2C2CNC(=O)C2)cc1-c1cc2c(cc1F)C(=O)NCC2] |
| I-480 | 6-(2-amino-5-(3- ((dimethylamino)methyl)-4- morpholinophenyl)-6- fluoropyridin-3-yl)-4- fluoroisoquinolin-1(2H)-one | 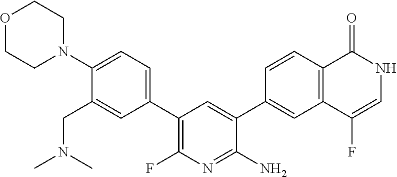 [SMILES: CN(C)Cc1cc(-c2cc(-c3ccc4c(=O)[nH]cc(F)c4c3)c(N)nc2F)ccc1N1CCOCC1] |
| I-481 | 6-(2-amino-6-fluoro-5-(4-(4- isopropylpiperazin-1- yl)phenyl)pyridin-3-yl)-4,8- difluoro-3-methylisoquinolin- 1(2H)-one | 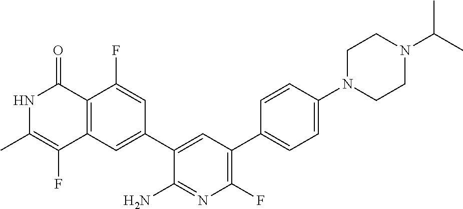 [SMILES: Cc1[nH]c(=O)c2c(F)cc(-c3cc(-c4ccc(N5CCN(C(C)C)CC5)cc4)c(F)nc3N)cc2c1F] |
| I-482 | 6-(2-amino-6-fluoro-5-(4-(4- isopropylpiperazin-1- yl)phenyl)pyridin-3-yl)-4,8- difluoroisoquinolin-1(2H)- one | 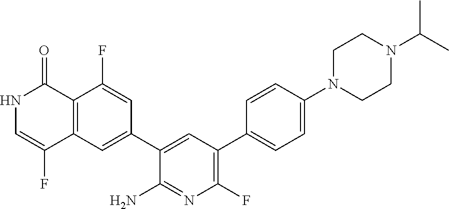 [SMILES: CC(C)N1CCN(c2ccc(-c3cc(-c4cc(F)c5c(=O)[nH]cc(F)c5c4)c(N)nc3F)cc2)CC1] |
| I-483 | 6-(2-amino-5-(4-(4- (cyclopropylmethyl)piperazin- 1-yl)phenyl)-6-fluoropyridin- 3-yl)-4,8-difluoroisoquinolin- 1(2H)-one | 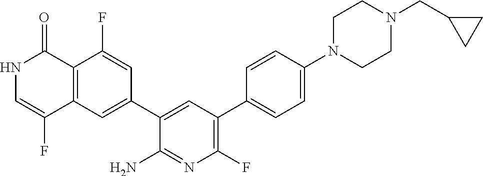 [SMILES: Nc1nc(F)c(-c2ccc(N3CCN(CC4CC4)CC3)cc2)cc1-c1cc(F)c2c(=O)[nH]cc(F)c2c1] |
| I-484 | 7-(2-amino-5-(4-(4-(4,4- difluorobutyl)piperazin-1- yl)phenyl)-6-fluoropyridin-3- yl)-2-methylquinazolin- 4(3H)-one | 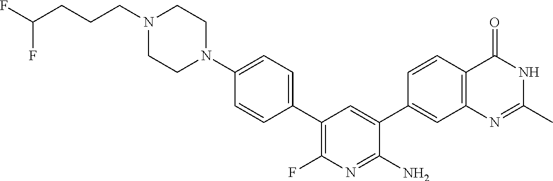 [SMILES: Cc1nc2cc(-c3cc(-c4ccc(N5CCN(CCCC(F)F)CC5)cc4)c(F)nc3N)ccc2c(=O)[nH]1] |
| I-485 | 7-(2-amino-5-(4-(4-(3,3- difluoropropyl)piperazin-1- yl)phenyl)-6-fluoropyridin-3- yl)-5-fluoro-2- methylquinazolin-4(3H)-one | 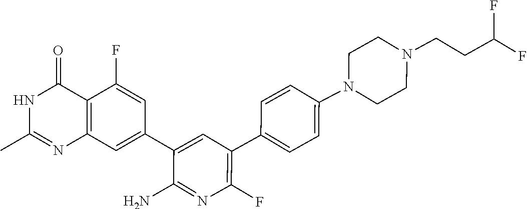 [SMILES: Cc1nc2cc(-c3cc(-c4ccc(N5CCN(CCC(F)F)CC5)cc4)c(F)nc3N)cc(F)c2c(=O)[nH]1] |
| I-486 | 6-(2-amino-5-(3- ((dimethylamino)methyl)-4- morpholinophenyl)-6- fluoropyridin-3-yl)-8-fluoro- 3,4-dihydroisoquinolin- 1(2H)-one | 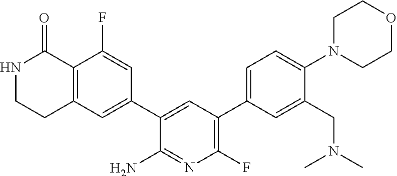 [SMILES: CN(C)Cc1cc(-c2cc(-c3cc(F)c4c(c3)CCNC4=O)c(N)nc2F)ccc1N1CCOCC1] |
| I-487 | 6-(2-amino-5-(3- ((dimethylamino)methyl)-4- thiomorpholinophenyl)-6- fluoropyridin-3-yl)-3,4- dihydroisoquinolin-1(2H)- one | 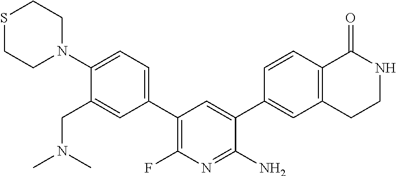 [SMILES: CN(C)Cc1cc(-c2cc(-c3ccc4c(c3)CCNC4=O)c(N)nc2F)ccc1N1CCSCC1] |
| I-488 | 6-(2-amino-5-(3- ((dimethylamino)methyl)-4- thiomorpholinophenyl)-6- fluoropyridin-3-yl)-7-fluoro- 3,4-dihydroisoquinolin- 1(2H)-one | 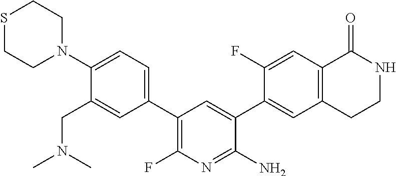 [SMILES: CN(C)Cc1cc(-c2cc(-c3cc4c(cc3F)C(=O)NCC4)c(N)nc2F)ccc1N1CCSCC1] |
| I-489 | 6-(2-amino-6-fluoro-5-(4-(4- isopropylpiperazin-1- yl)phenyl)pyridin-3-yl)-4- chloro-8-fluoroisoquinolin- 1(2H)-one | 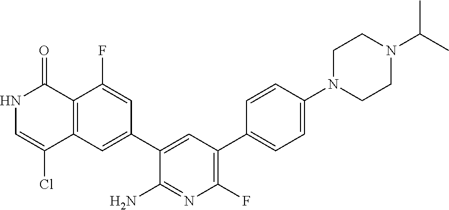 |
| I-490 | 7-(2-amino-5-(3- ((dimethylamino)methyl)-4- (tetrahydro-2H-pyran-4- yl)phenyl)-6-fluoropyridin-3- yl)-5-fluoro-2- methylquinazolin-4(3H)-one | 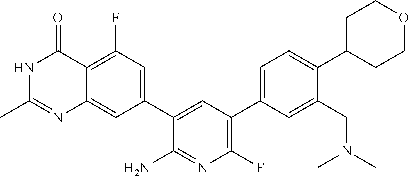 [SMILES: Cc1nc2cc(-c3cc(-c4ccc(C5CCOCC5)c(CN(C)C)c4)c(F)nc3N)cc(F)c2c(=O)[nH]1] |
| I-491 | 6-(2-amino-6-fluoro-5-(4- morpholino-3-(pyrrolidin-1- ylmethyl)phenyl)pyridin-3- yl)-3,4-dihydroisoquinolin- 1(2H)-one | 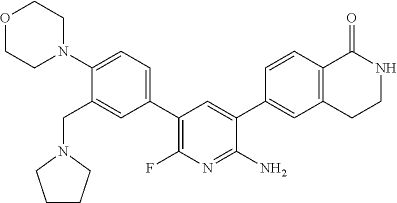 [SMILES: Nc1nc(F)c(-c2ccc(N3CCOCC3)c(CN3CCCC3)c2)cc1-c1ccc2c(c1)CCNC2=O] |
| I-492 | 6-(2-amino-6-fluoro-5- (4-morpholino-3- (pyrrolidin-1- ylmethyl)phenyl)pyridin- 3-yl)-7-fluoro-3,4- dihydroisoquinolin- 1(2H)-one | 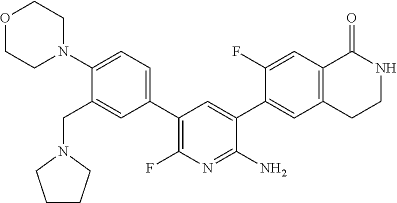 [SMILES: Nc1nc(F)c(-c2ccc(N3CCOCC3)c(CN3CCCC3)c2)cc1-c1cc2c(cc1F)C(=O)NCC2] |
| I-493 | 6-(2-amino-5-(3- ((dimethylamino)methyl)-4- (1,1- dioxidothiomorpholino)phenyl)- 6-fluoropyridin-3-yl)-3,4- dihydroisoquinolin-1(2H)- one | 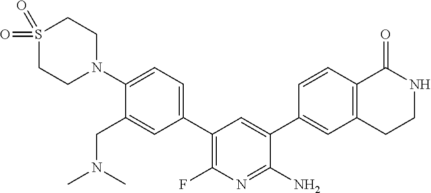 [SMILES: CN(C)Cc1cc(-c2cc(-c3ccc4c(c3)CCNC4=O)c(N)nc2F)ccc1N1CCS(=O)(=O)CC1] |
| I-494 | 6-(2-amino-5-(4-(4- cyclopropylpiperazin-1-yl)-3- ((dimethylamino)methyl)phe- nyl)-6-fluoropyridin-3-yl)-7- fluoro-3,4- dihydroisoquinolin-1(2H)- one | 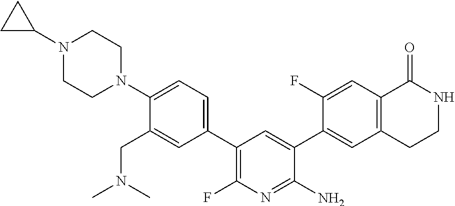 [SMILES: CN(C)Cc1cc(-c2cc(-c3cc4c(cc3F)C(=O)NCC4)c(N)nc2F)ccc1N1CCN(C2CC2)CC1] |
| I-495 | 6-(2-amino-5-(4-(4- cyclopropylpiperazin-1-yl)-3- ((dimethylamino)methyl)phe- nyl)-6-fluoropyridin-3-yl)-3,4- dihydroisoquinolin-1(2H)- one | 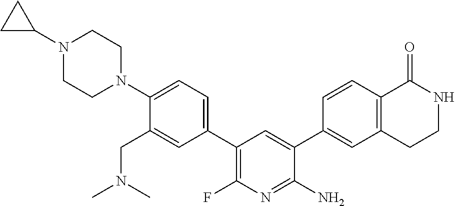 [SMILES: CN(C)Cc1cc(-c2cc(-c3ccc4c(c3)CCNC4=O)c(N)nc2F)ccc1N1CCN(C2CC2)CC1] |
| I-496 | 6-(2-amino-6-fluoro-5-(4- morpholino-3- (morpholinomethyl)phenyl)py- ridin-3-yl)-3,4- dihydroisoquinolin-1(2H)- one | 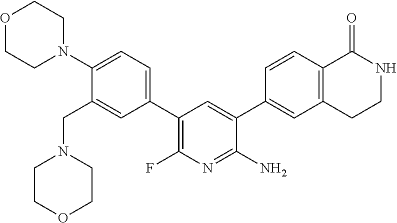 [SMILES: Nc1nc(F)c(-c2ccc(N3CCOCC3)c(CN3CCOCC3)c2)cc1-c1ccc2c(c1)CCNC2=O] |
| I-497 | 6-(2-amino-6-fluoro-5-(4- morpholino-3- (morpholinomethyl)phenyl)py- ridin-3-yl)-7-fluoro-3,4- dihydroisoquinolin-(2H)- one | 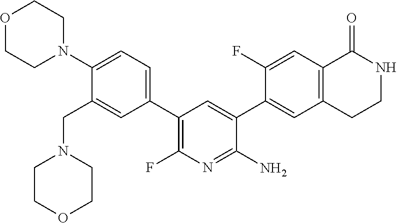 [SMILES: Nc1nc(F)c(-c2ccc(N3CCOCC3)c(CN3CCOCC3)c2)cc1-c1cc2c(cc1F)C(=O)NCC2] |
| I-498 | 6-(2-amino5-(3- ((dimethylamino)methyl)-4- (1,1- dioxidothiomorpholino)phenyl)- 6-fluoropyridin-3-yl)-7- fluoro-3,4- dihydroisoquinolin-1(2H)- one | 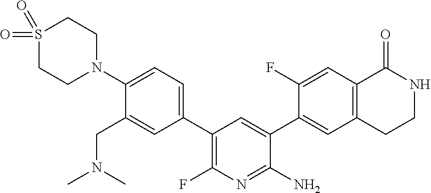 [SMILES: CN(C)Cc1cc(-c2cc(-c3cc4c(cc3F)C(=O)NCC4)c(N)nc2F)ccc1N1CCS(=O)(=O)CC1] |
| I-499 | 6-(2-amino-6-fluoro-5-(4- ((3R,5S)-3,4,5- trimethylpiperazin-1- yl)phenyl)pyridin-3-yl)-4- fluoroisoquinolin-1(2H)-one | 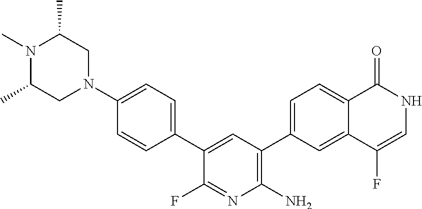 [SMILES: C[C@@H]1CN(c2ccc(-c3cc(-c4ccc5c(=O)[nH]cc(F)c5c4)c(N)nc3F)cc2)C[C@H](C)N1C] |
| I-500 | 7-(2-amino-6-fluoro-5-(4- ((3R,5S)-3,4,5- trimethylpiperazin-1- yl)phenyl)pyridin-3-yl)-2- methylquinazolin-4(3H)-one | 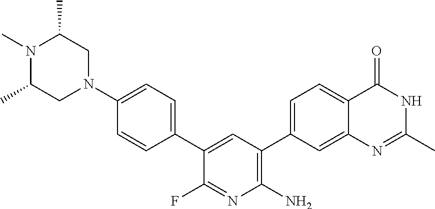 |
| I-501 | 7-(2-amino-6-fluoro-5-(4- ((3R,5S)-3,4,5- trimethylpiperazin-1- yl)phenyl)pyridin-3-yl)-5- fluoro-2-methylquinazolin- 4(3H)-one | 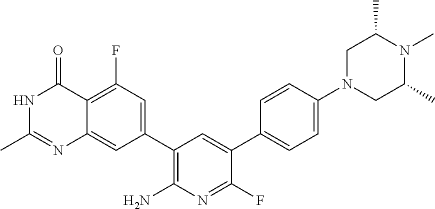 |
| I-502 | 6-(2-amino-5-(3- ((dimethylamino)methyl)-4- morpholinophenyl)-6- fluoropyridin-3-yl)-4- methylisoquinolin-1(2H)-one | 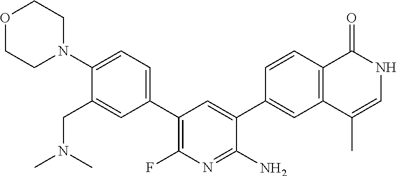 [SMILES: Cc1c[nH]c(=O)c2ccc(-c3cc(-c4ccc(N5CCOCC5)c(CN(C)C)c4)c(F)nc3N)cc12] |
| I-503 | 6-(2-amino-5-(3- ((dimethylamino)methyl)-4- (tetrahydro-2H-pyran-4- yl)phenyl)-6-fluoropyridin-3- yl)-4-fluoroisoquinolin-1(2H)- one | 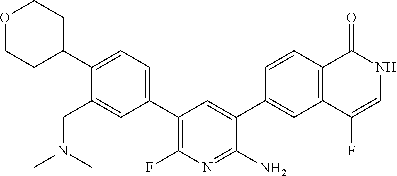 [SMILES: CN(C)Cc1cc(-c2cc(-c3ccc4c(=O)[nH]cc(F)c4c3)c(N)nc2F)ccc1C1CCOCC1] |
| I-504 | 6-(2-amino-5-(3- ((dimethylamino)methyl)-4- (tetrahydro-2H-pyran-4- y)phenyl)-6-fluoropyridin-3- yl)-8-fluoro-3- methylisoquinolin-1(2H)-one | 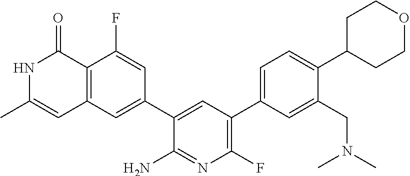 [SMILES: Cc1cc2cc(-c3cc(-c4ccc(C5CCOCC5)c(CN(C)C)c4)c(F)nc3N)cc(F)c2c(=O)[nH]1] |
| I-505 | 6-(2-amino-5-(3- ((dimethylamino)methyl)-4- morpholinophenyl)-6- fluoropyridin-3-yl)-8-fluoro-3- methylisoquinolin-1(2H)-one | 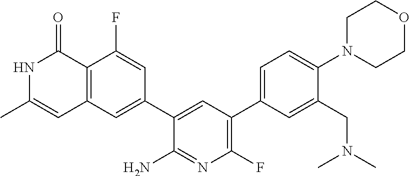 [SMILES: Cc1cc2cc(-c3cc(-c4ccc(N5CCOCC5)c(CN(C)C)c4)c(F)nc3N)cc(F)c2c(=O)[nH]1] |
| I-506 | 6-(2-amino-5-(4- (cyclopropylmethoxy)-3- ((dimethylamino)methyl)phe- nyl)-6-fluoropyridin-3-yl)-3,4- dihydroisoquinolin-1(2H)- one | 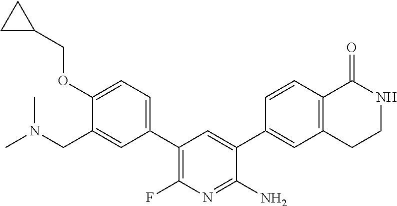 [SMILES: CN(C)Cc1cc(-c2cc(-c3ccc4c(c3)CCNC4=O)c(N)nc2F)ccc1OCC1CC1] |
| I-507 | 6-(2-amino-5-(4- (cyclopropylmethoxy)-3- ((dimethylamino)methyl)phe- nyl)-6-fluoropyridin-3-yl)-7- fluoro-3,4- dihydroisoquinolin-1(2H)- one | 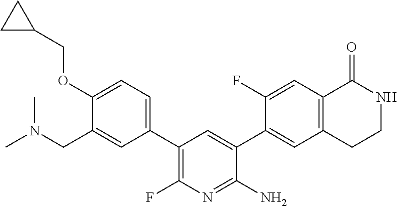 [SMILES: CN(C)Cc1cc(-c2cc(-c3cc4c(cc3F)C(=O)NCC4)c(N)nc2F)ccc1OCC1CC1] |
| I-508 | 6-(2-amino-5-(4- (cyclopropylmethoxy)-3-((4- methoxypiperidin-1- yl)methyl)phenyl)-6- fluoropyridin-3-yl)-3,4- dihydroisoquinolin-1(2H)- one | 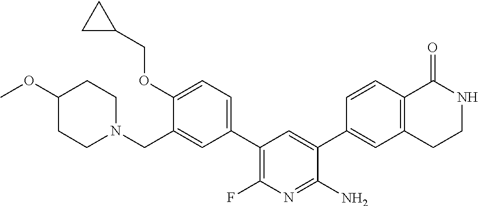 [SMILES: COC1CCN(Cc2cc(-c3cc(-c4ccc5c(c4)CCNC5=O)c(N)nc3F)ccc2OCC2CC2)CC1] |
| I-509 | 6-(2-amino-5-(4- (cyclopropylmethoxy)-3-((4- methoxypiperidin-1- yl)methyl)phenyl)-6- fluoropyridin-3-yl)-7-fluoro- 3,4-dihydroisoquinolin- 1(2H)-one | 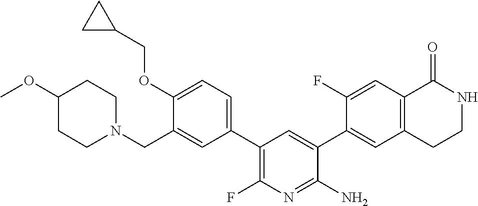 [SMILES: COC1CCN(Cc2cc(-c3cc(-c4cc5c(cc4F)C(=O)NCC5)c(N)nc3F)ccc2OCC2CC2)CC1] |
| I-510 | 6-(2-amino-6-fluoro-5-(3-((4- methoxypiperidin-1- yl)methyl)-4- morpholinophenyl)pyridin-3- yl)-3,4-dihydroisoquinolin- 1(2H)-one | 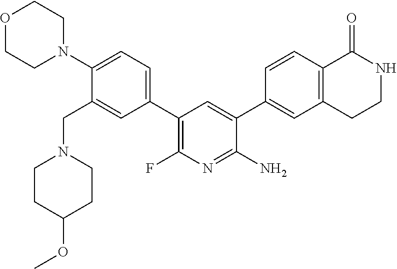 [SMILES: COC1CCN(Cc2cc(-c3cc(-c4ccc5c(c4)CCNC5=O)c(N)nc3F)ccc2N2CCOCC2)CC1] |
| I-511 | 6-(2-amino-6-fluoro-5-(3-((4- methoxypiperidin-1- yl)methyl)-4- morpholinophenyl)pyridin-3- yl)-7-fluoro-3,4- dihydroisoquinolin-1(2H)- one | 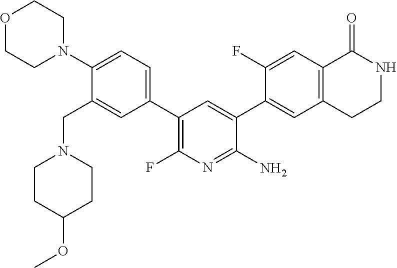 [SMILES: COC1CCN(Cc2cc(-c3cc(-c4cc5c(cc4F)C(=O)NCC5)c(N)nc3F)ccc2N2CCOCC2)CC1] |
| I-512 | 6-(2-amino-6-fluoro-5-(3-((4- methoxypiperidin-1- yl)methyl)-4- thiomorpholinophenyl)pyridin- 3-yl)-3,4- dihydroisoquinolin-1(2H)- one | 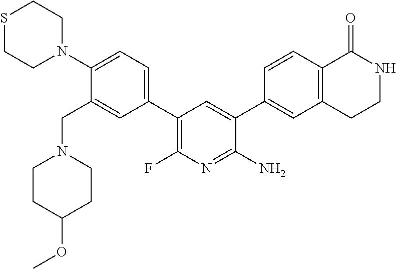 [SMILES: COC1CCN(Cc2cc(-c3cc(-c4ccc5c(c4)CCNC5=O)c(N)nc3F)ccc2N2CCSCC2)CC1] |
| I-513 | 6-(2-amino-6-fluoro-5-(3-((4- methoxypiperidin-1- yl)methyl)-4- thiomorpholinophenyl)pyridin- 3-yl)-7-fluoro-3,4- dihydroisoquinolin-1(2H)- one | 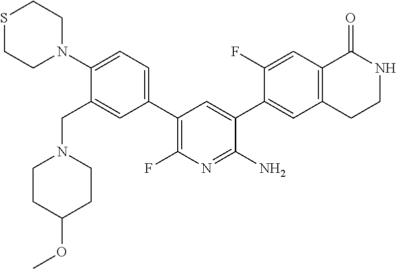 [SMILES: COC1CCN(Cc2cc(-c3cc(-c4cc5c(cc4F)C(=O)NCC5)c(N)nc3F)ccc2N2CCSCC2)CC1] |
| I-514 | 2-(6-amino-2-fluoro-5-(7- fluoro-1-oxo-1,2,3,4- tetrahydroisoquinolin-6- yl)pyridin-3-yl)-5-(3,6- dihydro-2H-pyran-4- yl)benzaldehyde | 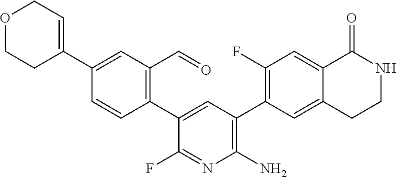 [SMILES: Nc1nc(F)c(-c2ccc(C3=CCOCC3)cc2C=O)cc1-c1cc2c(cc1F)C(=O)NCC2] |
| I-515 | 6-(2-amino-5-(3- ((dimethylamino)methyl)-4- morpholinophenyl)-6- fluoropyridin-3-yl)-4-fluoro-3- methylisoquinolin-1(2H)-one | 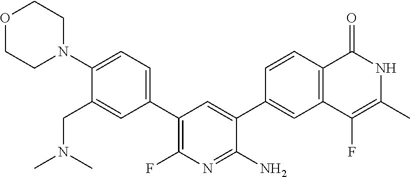 [SMILES: Cc1[nH]c(=O)c2ccc(-c3cc(-c4ccc(N5CCOCC5)c(CN(C)C)c4)c(F)nc3N)cc2c1F] |
| I-516 | 6-(2-amino-6-fluoro-5-(4- methoxy-3-((3- methoxyazetidin-1- yl)methyl)phenyl)pyridin-3- yl)-3,4-dihydroisoquinolin- 1(2H)-one | 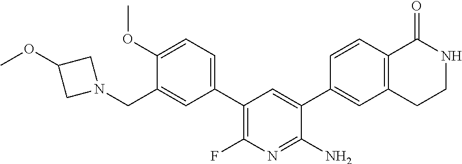 [SMILES: COc1ccc(-c2cc(-c3ccc4c(c3)CCNC4=O)c(N)nc2F)cc1CN1CC(OC)C1] |
| I-517 | 6-(2-amino-6-fluoro-5-(4- methoxy-3-((3- methoxyazetidin-1- yl)methyl)phenyl)pyridin-3- yl)-7-fluoro-3,4- dihydroisoquinolin-1(2H)- one | 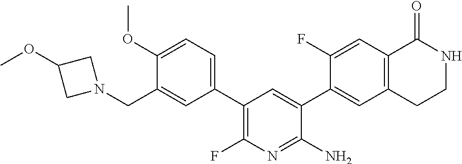 [SMILES: COc1ccc(-c2cc(-c3cc4c(cc3F)C(=O)NCC4)c(N)nc2F)cc1CN1CC(OC)C1] |
| I-518 | 6-(2-amino-6-fluoro-5-(4- methoxy-3-((4- methoxypiperidin-1- yl)methyl)phenyl)pyridin-3- yl)-3,4-dihydroisoquinolin- 1(2H)-one | 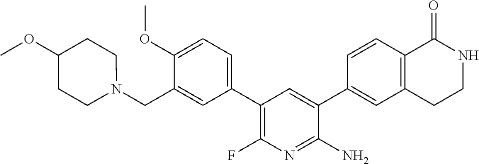 [SMILES: COc1ccc(-c2cc(-c3ccc4c(c3)CCNC4=O)c(N)nc2F)cc1CN1CCC(OC)CC1] |
| I-519 | 6-(2-amino-6-fluoro-5-(4- methoxy-3-((4- methoxypiperidin-1- yl)methyl)phenyl)pyridin-3- yl)-7-fluoro-3,4- dihydroisoquinolin-1(2H)- one | 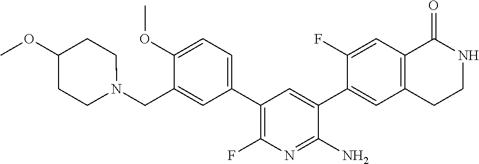 [SMILES: COc1ccc(-c2cc(-c3cc4c(cc3F)C(=O)NCC4)c(N)nc2F)cc1CN1CCC(OC)CC1] |
| I-520 | 6-(2-amino-5-(4-((1S,5R)-3- cyclobutyl-3- azabicyclo[3.1.0]hexan-1- yl)phenyl)-6-fluoropyridin-3- yl)-4-fluoroisoquinolin-1(2H)- one | 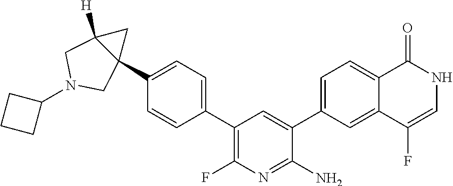 [SMILES: Nc1nc(F)c(-c2ccc([C@]34C[C@H]3CN(C3CCC3)C4)cc2)cc1-c1ccc2c(=O)[nH]cc(F)c2c1] |
| I-521 | 6-(2-amino-5-(3- ((dimethylamino)methyl)-4- (tetrahydro-2H-pyran-4- yl)phenyl)-6-fluoropyridin-3- yl)-4-fluoro-3- methylisoquinolin-1(2H)-one | 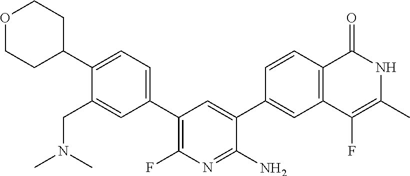 [SMILES: Cc1[nH]c(=O)c2ccc(-c3cc(-c4ccc(C5CCOCC5)c(CN(C)C)c4)c(F)nc3N)cc2c1F] |
| I-522 | 6-(2-amino-6-fluoro-5-(3-((3- methoxyazetidin-1- yl)methyl)-4- morpholinophenyl)pyridin-3- yl)-3,4-dihydroisoquinolin- 1(2H)-one | 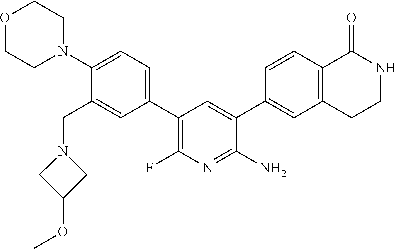 [SMILES: COC1CN(Cc2cc(-c3cc(-c4ccc5c(c4)CCNC5=O)c(N)nc3F)ccc2N2CCOCC2)C1] |
| I-523 | 6-(2-amino-6-fluoro-5-(3-((3- methoxyazetidin-1- yl)methyl)-4- morpholinophenyl)pyridin-3- yl)-7-fluoro-3,4- dihydroisoquinolin-1(2H)- one | 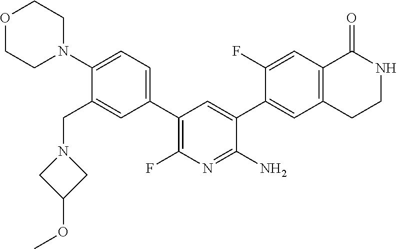 [SMILES: COC1CN(Cc2cc(-c3cc(-c4cc5c(cc4F)C(=O)NCC5)c(N)nc3F)ccc2N2CCOCC2)C1] |
| I-524 | 6-(2-amino-5-(4-(4,4- difluorobutoxy)-3- ((dimethylamino)methyl)phe- nyl)-6-fluoropyridin-3-yl)-3,4- dihydroisoquinolin-1(2H)- one | 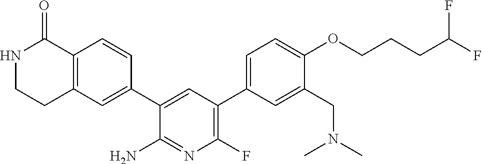 [SMILES: CN(C)Cc1cc(-c2cc(-c3ccc4c(c3)CCNC4=O)c(N)nc2F)ccc1OCCCC(F)F] |
| I-525 | 6-(2-amino-5-(4-(4,4- difluorobutoxy)-3- ((dimethylamino)methyl)phe- nyl)-6-fluoropyridin-3-yl)-7- fluoro-3,4- dihydroisoquinolin-1(2H)- one | 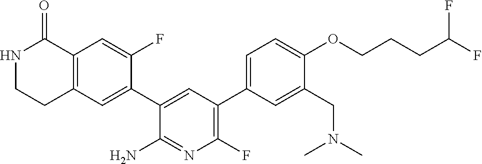 [SMILES: CN(C)Cc1cc(-c2cc(-c3cc4c(cc3F)C(=O)NCC4)c(N)nc2F)ccc1OCCCC(F)F] |
| I-526 | 6-(2-amio-5-(4-(4,4- difluorobutoxy)-3-((4- methoxypiperidin-1- yl)methyl)phenyl)-6- fluoropyridin-3-yl)-3,4- dihydroisoquinolin-1(2H)- one | 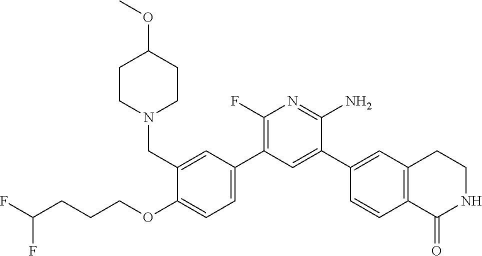 [SMILES: COC1CCN(Cc2cc(-c3cc(-c4ccc5c(c4)CCNC5=O)c(N)nc3F)ccc2OCCCC(F)F)CC1] |
| I-527 | 6-(2-amino-5-(4-(4,4- difluorobutoxy)-3-((4- methoxypiperidin-1- yl)methyl)phenyl)-6- fluoropyridin-3-yl)-7-fluoro- 3,4-dihydroisoquinolin- 1(2H)-one | 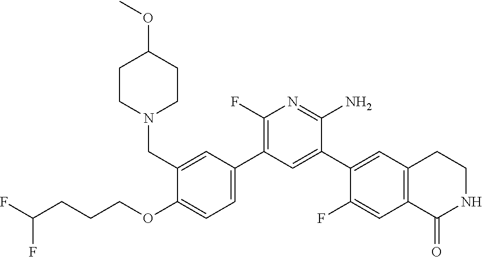 [SMILES: COC1CCN(Cc2cc(-c3cc(-c4cc5c(cc4F)C(=O)NCC5)c(N)nc3F)ccc2OCCCC(F)F)CC1] |
| I-528 | 6-(2-amino-5-(4-(3,6- dihydro-2H-pyran-4-yl)-2- (hydroxymethyl)phenyl)-6- fluoropyridin-3-yl)-7-fluoro- 3,4-dihydroisoquinolin- 1(2H)-one | 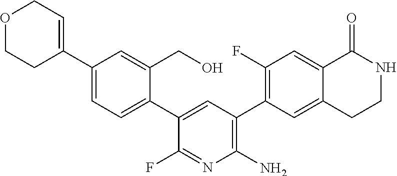 [SMILES: Nc1nc(F)c(-c2ccc(C3=CCOCC3)cc2CO)cc1-c1cc2c(cc1F)C(=O)NCC2] |
| I-529 | 6-(2-amino-5-(4-(4- (cyclopropylmethyl)piperazin- 1-yl)phenyl)-6-fluoropyridin- 3-yl)-4,8-difluoro-3- methylisoquinolin-1(2H)-one | 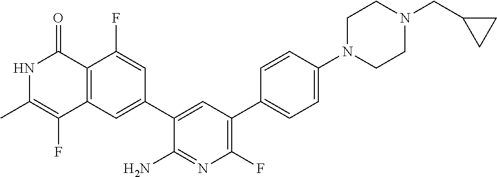 [SMILES: Cc1[nH]c(=O)c2c(F)cc(-c3cc(-c4ccc(N5CCN(CC6CC6)CC5)cc4)c(F)nc3N)cc2c1F] |
| I-530 | 6-(2-amino-5-(3- ((dimethylamino)methyl)-4- ((tetrahydro-2H-pyran-4- yl)oxy)phenyl)-6- fluoropyridin-3-yl)-3,4- dihydroisoquinolin-1(2H)- one | 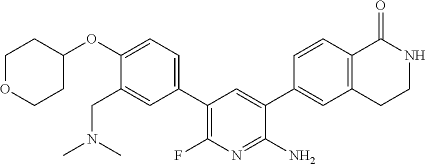 [SMILES: CN(C)Cc1cc(-c2cc(-c3ccc4c(c3)CCNC4=O)c(N)nc2F)ccc1OC1CCOCC1] |
| I-531 | 6-(2-amino-5-(3- ((dimethylamino)methyl)-4- ((tetrahydro-2H-pyran-4- yl)oxy)phenyl)-6- fluoropyridin-3-yl)-7-fluoro- 3,4-dihydroisoquinolin- 1(2H)-one | 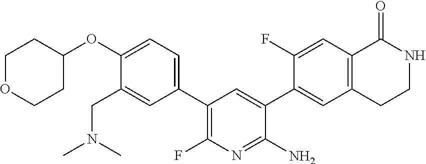 [SMILES: CN(C)Cc1cc(-c2cc(-c3cc4c(cc3F)C(=O)NCC4)c(N)nc2F)ccc1OC1CCOCC1] |
| I-532 | 6-(2-amino-5-(3- ((dimethylamino)methyl)-4- ((tetrahydro-2H-pyran-4- yl)methoxy)phenyl)-6- fluoropyridin-3-yl)-3,4- dihydroisoquinolin-1(2H)- one | 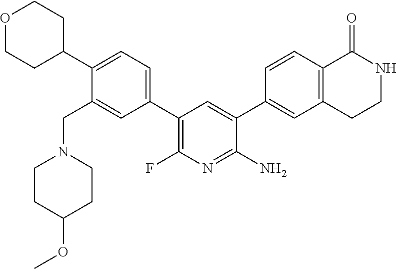 [SMILES: COC1CCN(Cc2cc(-c3cc(-c4ccc5c(c4)CCNC5=O)c(N)nc3F)ccc2C2CCOCC2)CC1] |
| I-533 | 6-(2-amino-5-(3- ((dimethylamino)methyl)-4- ((tetrahydro-2H-pyran-4- yl)methoxy)phenyl)-6- fluoropyridin-3-yl)-7-fluoro- 3,4-dihydroisoquinolin- 1(2H)-one | 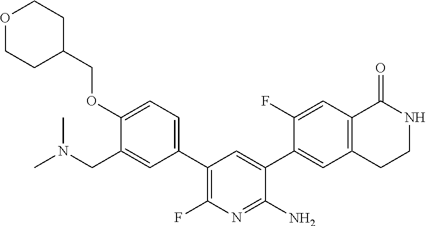 [SMILES: CN(C)Cc1cc(-c2cc(-c3cc4c(cc3F)C(=O)NCC4)c(N)nc2F)ccc1OCC1CCOCC1] |
| I-534 | 6-(2-amino-5-(3- ((dimethylamino)methyl)-4- morpholinophenyl)-6- fluoropyridin-3-yl)-4,8- difluoro-3-methylisoquinolin- 1(2H)-one | 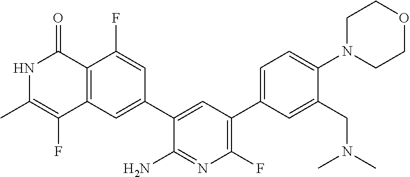 [SMILES: Cc1[nH]c(=O)c2c(F)cc(-c3cc(-c4ccc(N5CCOCC5)c(CN(C)C)c4)c(F)nc3N)cc2c1F] |
| I-535 | 6-(2-amino-5-(3- ((dimethylamino)methyl)-4- (tetrahydro-2H-pyran-4- yl)phenyl)-6-fluoropyridin-3- yl)-4,8-difluoro-3- methylisoquinolin-1(2H)-one | 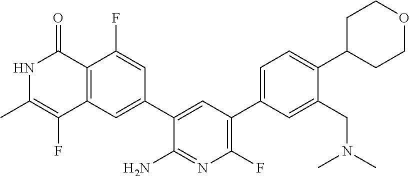 [SMILES: Cc1[nH]c(=O)c2c(F)cc(-c3cc(-c4ccc(C5CCOCC5)c(CN(C)C)c4)c(F)nc3N)cc2c1F] |
| I-536 | 6-(2-amino-6-fluoro-5-(3- (hydroxymethyl)-4- methoxyphenyl)pyridin-3-yl)- 3,4-dihydroisoquinolin- 1(2H)-one | 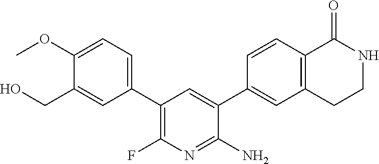 [SMILES: COc1ccc(-c2cc(-c3ccc4c(c3)CCNC4=O)c(N)nc2F)cc1CO] |
| I-537 | 6-(2-amino-6-fluoro-5-(4-(4- isopropylpiperazin-1- yl)phenyl)pyridin-3-yl)-4- methoxyisoquinolin-1(2H)- one | 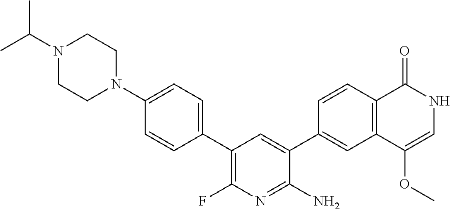 [SMILES: COc1c[nH]c(=O)c2ccc(-c3cc(-c4ccc(N5CCN(C(C)C)CC5)cc4)c(F)nc3N)cc12] |
| I-538 | 6-(2-amino-6-fluoro-5-(4-(4- isopropylpiperazin-1- yl)phenyl)pyridin-3-yl)-4- chloro-3-methylisoquinolin- 1(2H)-one | 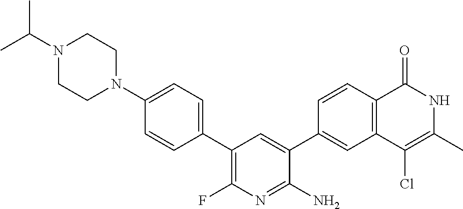 [SMILES: Cc1[nH]c(=O)c2ccc(-c3cc(-c4ccc(N5CCN(C(C)C)CC5)cc4)c(F)nc3N)cc2c1Cl] |
| I-539 | 6-(2-amino-6-fluoro-5-(4-(8- methyl-5-oxa-2,8- diazaspiro[3.5]nonan-2- yl)phenyl)pyridin-3-yl)-7- fluoro-3,4- dihydroisoquinolin-1(2H)- one | 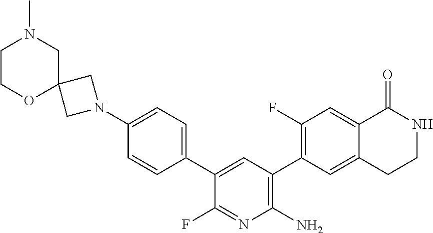 [SMILES: CN1CCOC2(C1)CN(c1ccc(-c3cc(-c4cc5c(cc4F)C(=O)NCC5)c(N)nc3F)cc1)C2] |
| I-540 | 6-(2-amino-6-fluoro-5-(3-((4- methoxpiperidin-1- yl)methyl)-4-(tetrahydro-2H- pryan-4-yl)phenyl)pyridin-3- yl)-3,4-dihydroisoquinolin- 1(2H)-one | 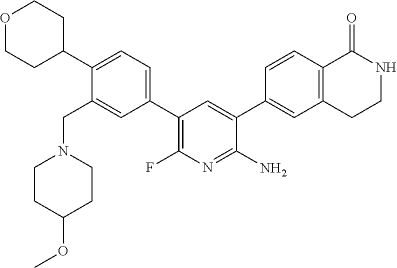 [SMILES: COC1CCN(Cc2cc(-c3cc(-c4ccc5c(c4)CCNC5=O)c(N)nc3F)ccc2C2CCOCC2)CC1] |
| I-541 | 6-(2-amino-6-fluoro-5-(3-((4- methoxypiperidin-1- yl)methyl)-4-(tetrahydro-2H- pyran-4-yl)phenyl)pyridin-3- yl)-7-fluoro-3,4- dihydroisoquinolin-1(2H)- one | 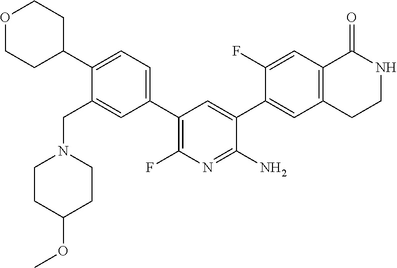 [SMILES: COC1CCN(Cc2cc(-c3cc(-c4cc5c(cc4F)C(=O)NCC5)c(N)nc3F)ccc2C2CCOCC2)CC1] |
| I-542 | 6-(2-amino-6-fluoro-5-(3-((3- methoxypyrrolidin-1- yl)methyl)-4- morpholinophenyl)pyridin-3- yl)-3,4-dihydroisoquinolin- 1(2H)-one | 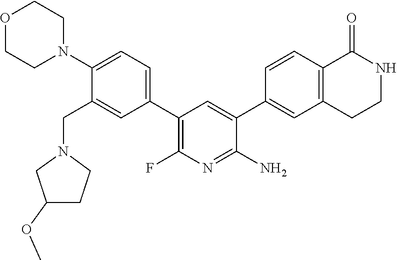 [SMILES: COC1CCN(Cc2cc(-c3cc(-c4ccc5c(c4)CCNC5=O)c(N)nc3F)ccc2N2CCOCC2)C1] |
| I-543 | 6-(2-amino-6-fluoro-5-(3-((3- methoxypyrrolidin-1- yl)methyl)-4- morpholinophenyl)pyridin-3- yl)-7-fluoro-3,4- dihydroisoquinolin-1(2H)- one | 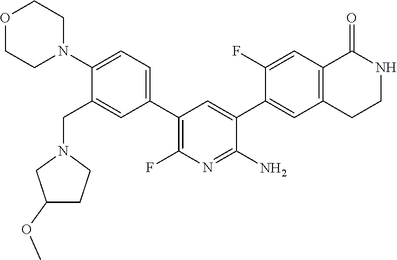 [SMILES: COC1CCN(Cc2cc(-c3cc(-c4cc5c(cc4F)C(=O)NCC5)c(N)nc3F)ccc2N2CCOCC2)C1] |
| I-544 | 6-(2-amino-5-(4-(4-(4,4- difluorobutyl)piperazin-1- yl)phenyl)-6-fluoropyridin-3- yl)-4-fluoroisoquinolin-1(2H)- one | 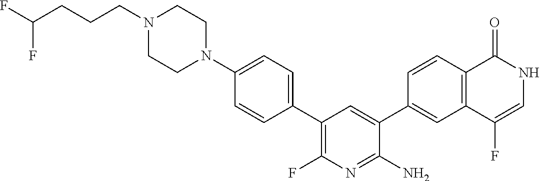 [SMILES: Nc1nc(F)c(-c2ccc(N3CCN(CCCC(F)F)CC3)cc2)cc1-c1ccc2c(=O)[nH]cc(F)c2c1] |
| I-545 | 6-(2-amino-5-(4-(4- cyclobutylpiperazin-1- yl)phenyl)-6-fluoropyriidn-3- yl)-4-methylisoquinolin- 1(2H)-one | 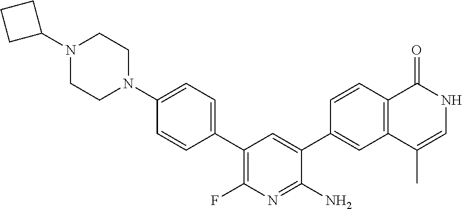 [SMILES: Cc1c[nH]c(=O)c2ccc(-c3cc(-c4ccc(N5CCN(C6CCC6)CC5)cc4)c(F)nc3N)cc12] |
| I-546 | 6-(2-amino-5-(4-(4- cyclobutylpiperazin-1- yl)phenyl)-6-fluoropyridin-3- yl)-4-fluoroisoquinolin-1(2H)- one | 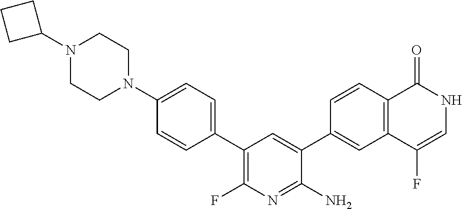 [SMILES: Nc1nc(F)c(-c2ccc(N3CCN(C4CCC4)CC3)cc2)cc1-c1ccc2c(=O)[nH]cc(F)c2c1] |
| I-547 | 6-(2-amino-6-fluoro-5-(4-(4- isopropylpiperazin-1- yl)phenyl)pyridin-3-yl)-4,7- diflurooisoquinolin-1(2H)- one | 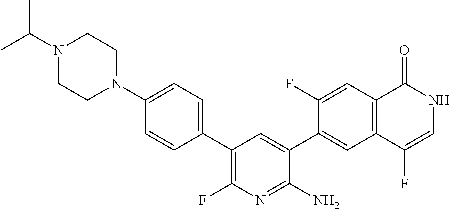 [SMILES: CC(C)N1CCN(c2ccc(-c3cc(-c4cc5c(F)c[nH]c(=O)c5cc4F)c(N)nc3F)cc2)CC1] |
| I-548 | 6-(2-amino-5-(4-(4- (cyclopropylmethyl)piperazin- 1-yl)phenyl)-6-fluoropyridin- 3-yl)-4,7-difluoroisoquinolin 1(2H)-one | 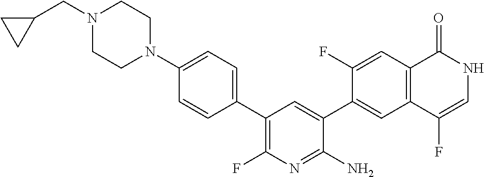 [SMILES: Nc1nc(F)c(-c2ccc(N3CCN(CC4CC4)CC3)cc2)cc1-c1cc2c(F)c[nH]c(=O)c2cc1F] |
| I-549 | 6-(2-amino-5-(3-(azetidin-1- ylmethyl)-4- morpholinophenyl)-6- fluoropyridin-3-yl)-3,4- dihydroisoquinolin-1(2H)- one | 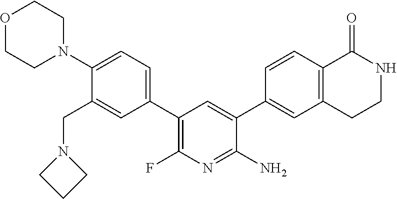 [SMILES: Nc1nc(F)c(-c2ccc(N3CCOCC3)c(CN3CCC3)c2)cc1-c1ccc2c(c1)CCNC2=O] |
| I-550 | 6-(2-amino-5-(3-(azetidin-1- ylmethyl)-4- morpholinophenyl)-6- fluoropyridin-3-yl)-7-fluroo- 3,4-dihydroisoquinolin- 1(2H)-one | 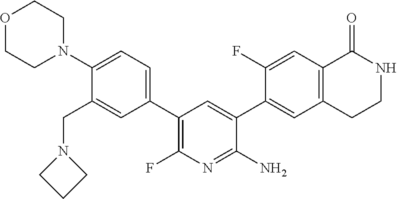 [SMILES: Nc1nc(F)c(-c2ccc(N3CCOCC3)c(CN3CCC3)c2)cc1-c1cc2c(cc1F)C(=O)NCC2] |
| I-551 | 6-(6′-amino-6- ((dimethylamino)methyl)-2′- fluoro-5-morpholino-[2,3′- bipyridin]-5′-yl)-3,4- dihydroisoquinolin-1(2H)- one | 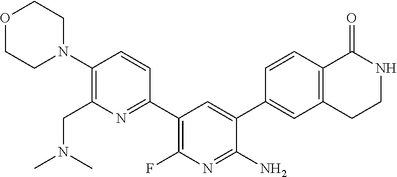 [SMILES: CN(C)Cc1nc(-c2cc(-c3ccc4c(c3)CCNC4=O)c(N)nc2F)ccc1N1CCOCC1] |
| I-552 | 6-(2-amino-5-(3- ((ethyl(methyl)amino)methyl)- 4-(tetrahydro-2H-pyran-4- yl)phenyl)-6-fluoropyridin-3- yl)-7-fluoro-3,4- dihydroisoquinolin-1(2H)- one | 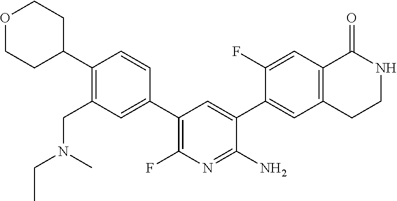 [SMILES: CCN(C)Cc1cc(-c2cc(-c3cc4c(cc3F)C(=O)NCC4)c(N)nc2F)ccc1C1CCOCC1] |
| I-553 | 6-(2-amino-6-fluoro-5-(3- (((2- methoxyethyl)(methyl)amino) methyl)-4- morpholinophenyl)pyridin-3- yl)-3,4-dihydroisoquinolin- 1(2H)-one | 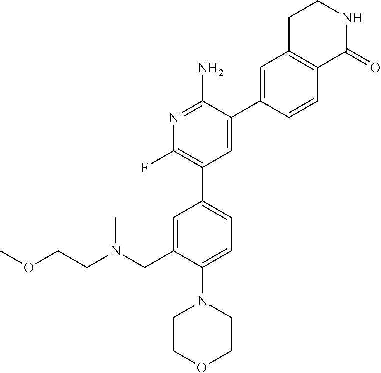 [SMILES: COCCN(C)Cc1cc(-c2cc(-c3ccc4c(c3)CCNC4=O)c(N)nc2F)ccc1N1CCOCC1] |
| I-554 | 65-(2-amino-6-fluoro-5-(3- (((2- methoxyethyl)(methyl)amino) methyl)-4- morpholinophenyl)pyridin-3- yl)-7-fluoro-3,4- dihydroisoquinolin-1(2H)- one | 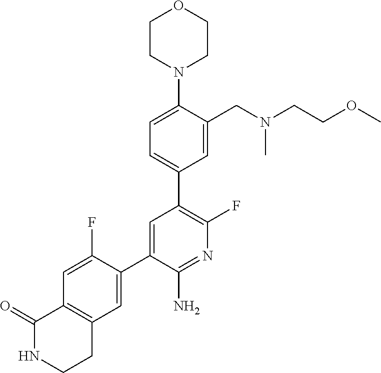 [SMILES: COCCN(C)Cc1cc(-c2cc(-c3cc4c(cc3F)C(=O)NCC4)c(N)nc2F)ccc1N1CCOCC1] |
| I-555 | 6-(2-amino-5-(4-(2,2- difluoroethoxy)-3- ((dimethylamino)methyl)phe- nyl)-6-fluoropyridin-3-yl)-3,4- dihydroisoquinolin-1(2H)- one | 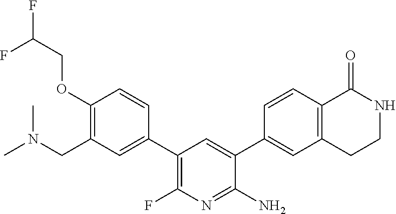 [SMILES: CN(C)Cc1cc(-c2cc(-c3ccc4c(c3)CCNC4=O)c(N)nc2F)ccc1OCC(F)F] |
| I-556 | 6-(2-amino-5-(4-(2,2- difluoroethoxy)-3- ((dimethylamino)methyl)phe- nyl)-6-fluoropyridin-3-yl)-7- fluoro-3,4- dihydroisoquinolin-1(2H)- one | 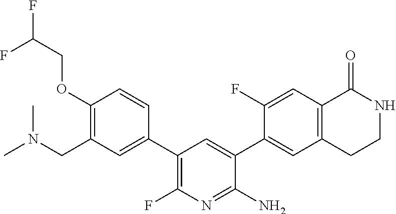 [SMILES: CN(C)Cc1cc(-c2cc(-c3cc4c(cc3F)C(=O)NCC4)c(N)nc2F)ccc1OCC(F)F] |
| I-557 | 6-(2-amino-6-fluoro-5-(3- (pyrrolidin-1-ylmethyl)-4- (tetrahydro-2H-pyran-4- yl)phenyl)pyridin-3-yl)-3,4- dihydroisoquinolin-1(2H)- one | 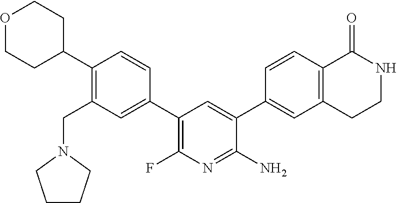 [SMILES: Nc1nc(F)c(-c2ccc(C3CCOCC3)c(CN3CCCC3)c2)cc1-c1ccc2c(c1)CCNC2=O] |
| I-558 | 6-(2-amino-6-fluoro-5-(3- (pyrrolidin-1-ylmethyl)-4- (tetrahydro-2H-pyran-4- yl)phenyl)pyridin-3-yl)-7- fluoro-3,4- dihydroisoquinolin-1(2H)- one | 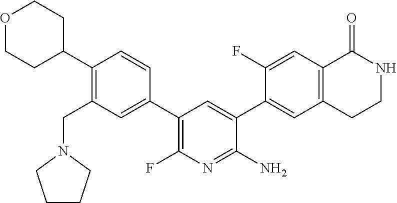 [SMILES: Nc1nc(F)c(-c2ccc(C3CCOCC3)c(CN3CCCC3)c2)cc1-c1cc2c(cc1F)C(=O)NCC2] |
| I-559 | 6-(2-amino-6-fluoro-5-(4- morpholino-3-(piperidin-1- ylmethyl)phenyl)pyridin-3- yl)-3,4-dihydroisoquinolin- 1(2H)-one | 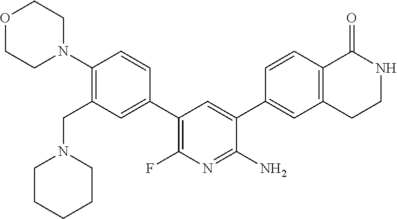 [SMILES: Nc1nc(F)c(-c2ccc(N3CCOCC3)c(CN3CCCCC3)c2)cc1-c1ccc2c(c1)CCNC2=O] |
| I-560 | 6-(2-amino-6-fluoro-5-(4- morpholino-3-(piperidin-1- ylmethyl)phenyl)pyridin-3- yl)-7-fluoro-3,4- dihydroisoquinolin-1(2H)- one | 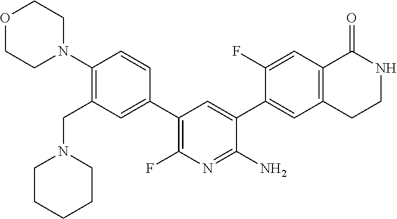 [SMILES: Nc1nc(F)c(-c2ccc(N3CCOCC3)c(CN3CCCCC3)c2)cc1-c1cc2c(cc1F)C(=O)NCC2] |
| I-561 | 6-(2-amino-5-(3- ((dimethylamino)methyl)-4- morpholinophenyl)-6- fluoropyridin-3-yl)-4,7- difluoroisoquinolin-1(2H)- one | 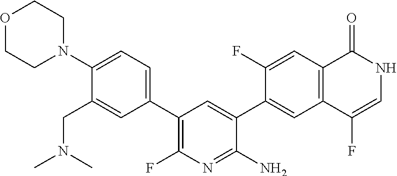 [SMILES: CN(C)Cc1cc(-c2cc(-c3cc4c(F)c[nH]c(=O)c4cc3F)c(N)nc2F)ccc1N1CCOCC1] |
| I-562 | 6-(2-amino-5-(3- ((dimethylamino)methyl)-4- (tetrahydro-2H-pyran-4- yl)phenyl)-6-fluoropyiridn-3- yl)-4,7-difluoroisoquinolin- 1(2H)-one | 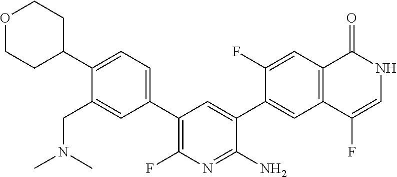 [SMILES: CN(C)Cc1cc(-c2cc(-c3cc4c(F)c[nH]c(=O)c4cc3F)c(N)nc2F)ccc1C1CCOCC1] |
| I-563 | (R)-6-(2-amino-6-fluoro-5-(3- ((3-methoxypyrrolidin-1- yl)methyl)-4- morpholinophenyl)pyridin-3- yl)-3,4-dihydroisoquinolin- 1(2H)-one | 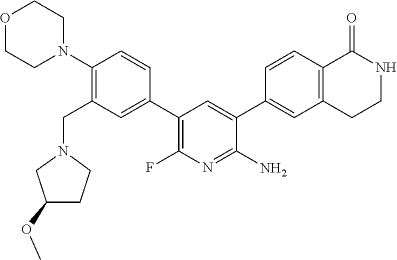 [SMILES: CO[C@@H]1CCN(Cc2cc(-c3cc(-c4ccc5c(c4)CCNC5=O)c(N)nc3F)ccc2N2CCOCC2)C1] |
| I-564 | (R)-6-(2-amino-6-fluoro-5-(3- ((3-methoxypyrrolidin-1- yl)methyl)-4- morpholinophenyl)pyridin-3- yl)-7-fluoro-3,4- dihydroisoquinolin-1(2H)- one | 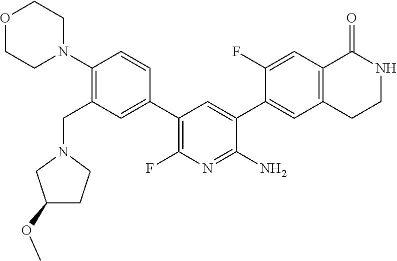 [SMILES: CO[C@@H]1CCN(Cc2cc(-c3cc(-c4cc5c(cc4F)C(=O)NCC5)c(N)nc3F)ccc2N2CCOCC2)C1] |
| I-565 | (S)-6-(2-amino-6-fluoro-5-(3- ((3-methoxypyrrolidin-1- yl)methyl)-4- morpholinophenyl)pyridin-3- yl)-3,4-dihydroisoquinolin- 1(2H)-one | 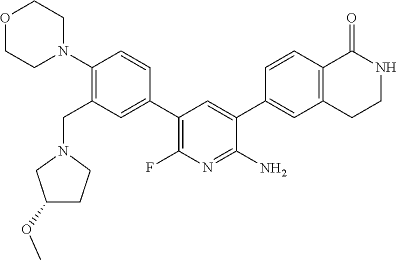 [SMILES: CO[C@H]1CCN(Cc2cc(-c3cc(-c4ccc5c(c4)CCNC5=O)c(N)nc3F)ccc2N2CCOCC2)C1] |
| I-566 | (S)-6-(2-amino-6-fluoro-5-(3- ((3-methoxypyrrolidin-1- yl)methyl)-4- morpholinophenyl)pyridin-3- yl)-7-fluoro-3,4- dihydroisoquinolin-1(1H)- one | 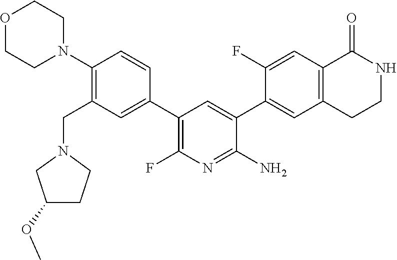 [SMILES: CO[C@H]1CCN(Cc2cc(-c3cc(-c4cc5c(cc4F)C(=O)NCC5)c(N)nc3F)ccc2N2CCOCC2)C1] |
| I-567 | 6-(2-amino-5-(4-(4-(4,4- difluorobutyl)piperazin-1- yl)phenyl)-6-fluoropyridin-3- yl)-8-fluoro-3- methylisoquinolin-1(2H)-one | 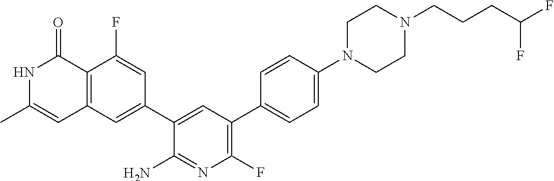 [SMILES: Cc1cc2cc(-c3cc(-c4ccc(N5CCN(CCCC(F)F)CC5)cc4)c(F)nc3N)cc(F)c2c(=O)[nH]1] |
| I-568 | 6-(5-(4-((1-acetylpiperidin-4- yl)oxy)-3- ((dimethylamino)methyl)phe- nyl)-2-amino-6-fluoropyridin- 3-yl)-3,4-dihydroisoquinolin- 1(2H)-one | 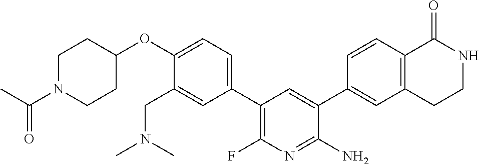 [SMILES: CC(=O)N1CCC(Oc2ccc(-c3cc(-c4ccc5c(c4)CCNC5=O)c(N)nc3F)cc2CN(C)C)CC1] |
| I-569 | 6-(5-(4-((1-acetylpiperidin-4- yl)oxy)-3- ((dimethylamino)methyl)phe- nyl)-2-amino-6-fluoropyridin- 3-yl)-7-fluoro-3,4- dihydroisoquinolin-1(2H)- one | 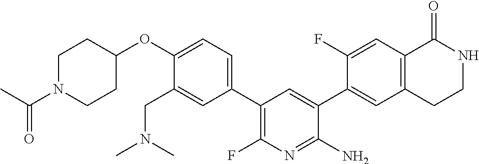 [SMILES: CC(=O)N1CCC(Oc2ccc(-c3cc(-c4cc5c(cc4F)C(=O)NCC5)c(N)nc3F)cc2CN(C)C)CC1] |
| I-570 | 6-(2-amino-6-fluoro-5-(4-(4- isopropylpiperazin-1- yl)phenyl)pyridin-3-yl)-4- chloro-2,7-naphthyridin- 1(2H)-one | 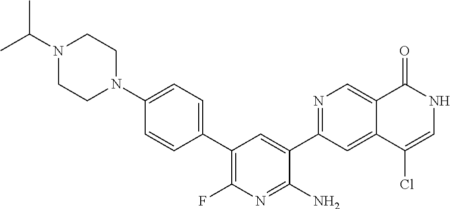 [SMILES: CC(C)N1CCN(c2ccc(-c3cc(-c4cc5c(Cl)c[nH]c(=O)c5cn4)c(N)nc3F)cc2)CC1] |
| I-571 | 6-(2-amino-6-fluoro-5-(4-(4- isopropylpiperazin-1- yl)phenyl)pyridin-3-yl)-8- fluoro-4-methylisoquinolin- 1(2H)-one | 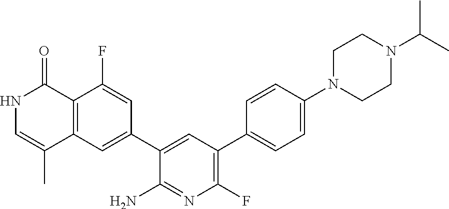 [SMILES: Cc1c[nH]c(=O)c2c(F)cc(-c3cc(-c4ccc(N5CCN(C(C)C)CC5)cc4)c(F)nc3N)cc12] |
| I-572 | 6-(2-amino-6-fluoro-5-(4-(4- isopropylpiperazin-1- yl)phenyl)pyriidn-3-yl)-4,7- difluoro-3-methylisoquinolin- 1(2H)-one | 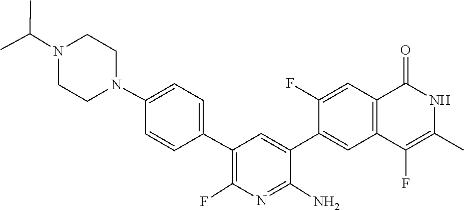 [SMILES: Cc1[nH]c(=O)c2cc(F)c(-c3cc(-c4ccc(N5CCN(C(C)C)CC5)cc4)c(F)nc3N)cc2c1F] |
| I-573 | 6-(2-amino-6-fluoro-5-(3- (((2- methoxyethyl)(methyl)amino) methyl)-4-(tetrahydro-2H- pyran-4-yl)phenyl)pyridin-3- yl)-3,4-dihydroisoquinolin- 1(2H)-one | 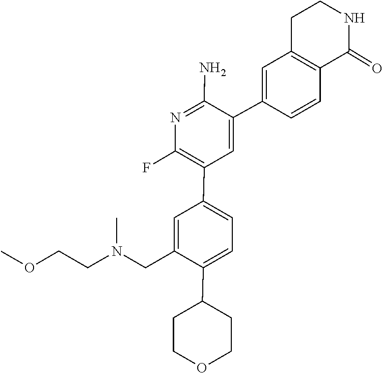 [SMILES: COCCN(C)Cc1cc(-c2cc(-c3ccc4c(c3)CCNC4=O)c(N)nc2F)ccc1C1CCOCC1] |
| I-574 | 6-(2-amino-6-fluoro-5-(3- (((2- methoxyethyl)(methyl)amino) methyl)-4-(tetrahydro-2H- pyran-4-yl)phenyl)pyridin-3- yl)-7-fluoro-3,4- dihydroisoquinolin-1(2H)- one | 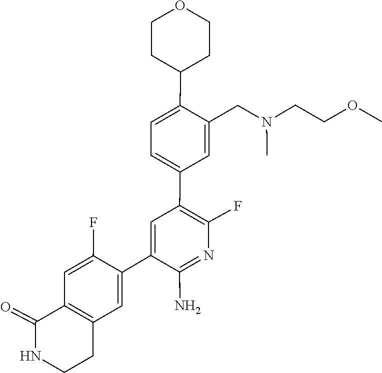 [SMILES: COCCN(C)Cc1cc(-c2cc(-c3cc4c(cc3F)C(=O)NCC4)c(N)nc2F)ccc1C1CCOCC1] |
| I-575 | (R)-6-(2-amino-5-(3- ((dimethylamino)methyl)-4- (2- methylmorpholino)phenyl)-6- fluoropyridin-3-yl)-3,4- dihydroisoquinolin-1(2H)- one | 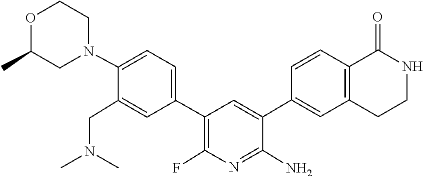 [SMILES: C[C@@H]1CN(c2ccc(-c3cc(-c4ccc5c(c4)CCNC5=O)c(N)nc3F)cc2CN(C)C)CCO1] |
| I-576 | (R)-6-(2-amino-5-(3- ((dimethylamino)methyl)-4- (2- methylmorpholino)phenyl)-6- fluoropyridin-3-yl)-7-fluoro- 3,4-dihydroisoquinolin- 1(2H)-one | 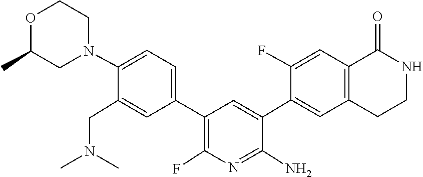 [SMILES: C[C@@H]1CN(c2ccc(-c3cc(-c4cc5c(cc4F)C(=O)NCC5)c(N)nc3F)cc2CN(C)C)CCO1] |
| I-577 | 6-(2-amino-6-fluoro-5-(4-(4- isopropylpiperazin-1- yl)phenyl)pyridin-3-yl)-7- fluoro-4-methylisoquinolin- 1(2H)-one | 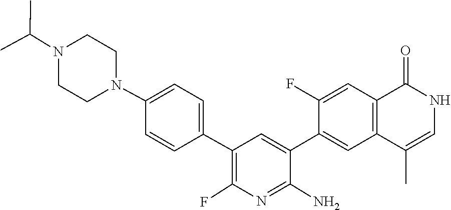 [SMILES: Cc1c[nH]c(=O)c2cc(F)c(-c3cc(-c4ccc(N5CCN(C(C)C)CC5)cc4)c(F)nc3N)cc12] |
| I-578 | 6-(6′-amino-6-(azetidin-1- ylmethyl)-2′-fluoro-5- morpholino-[2,3′-bipyridin]- 5′-yl)-3,4-dihydroisoquinolin- 1(2H)-one | 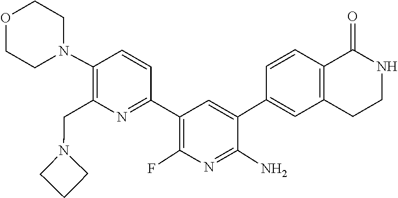 [SMILES: Nc1nc(F)c(-c2ccc(N3CCOCC3)c(CN3CCC3)n2)cc1-c1ccc2c(c1)CCNC2=O] |
| I-579 | 6-(6′-amino-6-(azetidin-1- ylmethyl)-2′-fluoro-5- morpholino-[2,3′-bipyridin]- 5′-yl)-7-fluoro-3,4- dihydroisoquinolin-1(2H)- one | 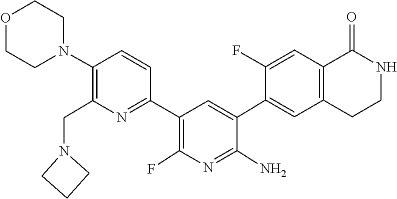 [SMILES: Nc1nc(F)c(-c2ccc(N3CCOCC3)c(CN3CCC3)n2)cc1-c1cc2c(cc1F)C(=O)NCC2] |
| I-580 | 6-(6′-amino-6- ((ethyl(methyl)amino)methyl)- 2′-fluoro-5-morpholino-[2,3′- bipyridin]-5′-yl)-3,4- dihydroisoquinolin-1(2H)- one | 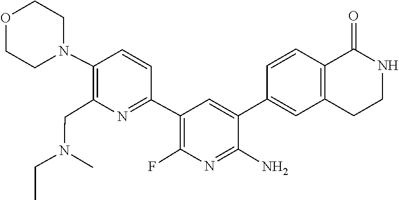 [SMILES: CCN(C)Cc1nc(-c2cc(-c3ccc4c(c3)CCNC4=O)c(N)nc2F)ccc1N1CCOCC1] |
| I-581 | 6-(6′-amino-6- ((ethyl(methyl)amino)methyl)- 2′-fluoro-5-morpholino-[2,3′- bipyridin]-5′-yl)-7-fluoro-3,4- dihydroisoquinolin-1(2H)- one | 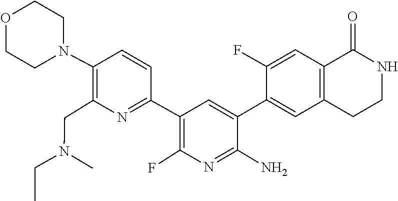 [SMILES: CCN(C)Cc1nc(-c2cc(-c3cc4c(cc3F)C(=O)NCC4)c(N)nc2F)ccc1N1CCOCC1] |
| I-582 | 6-(6′-amino-2′-fluoro-6-(((2- methoxyethyl)(methyl)amino) methyl)-5-morpholino-[2,3′- bipyridin]-5′-yl)-3,4- dihydroisoquinolin-1(2H)- one | 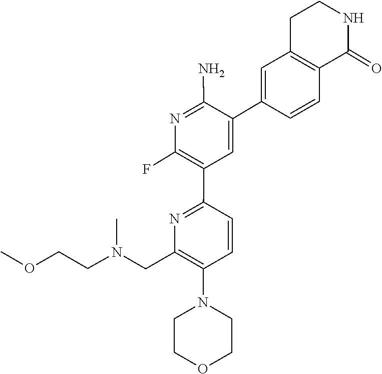 [SMILES: COCCN(C)Cc1nc(-c2cc(-c3ccc4c(c3)CCNC4=O)c(N)nc2F)ccc1N1CCOCC1] |
| I-583 | 6-(6′-amino-2′-fluoro-6-(((2- methoxyethyl)(methyl)amino) methyl)-5-morpholino-[2,3′- bipyridin]-5′-yl)-7-fluoro-3,4- dihydroisoquinolin-1(2H)- one | 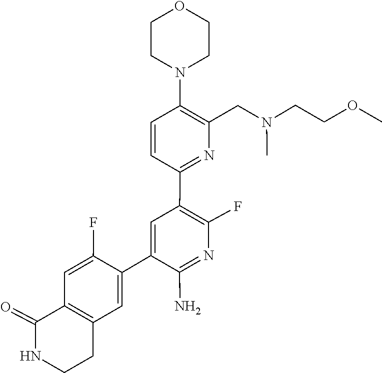 [SMILES: COCCN(C)Cc1nc(-c2cc(-c3cc4c(cc3F)C(=O)NCC4)c(N)nc2F)ccc1N1CCOCC1] |
| I-584 | (S)-6-(6′-amino-2′-fluoro-6- ((3-methoxypyrrolidin-1- yl)methyl)-5-morpholino- [2,3′-bipyridin]-5′-yl)-3,4- dihydroisoquinolin-1(2H)- one | 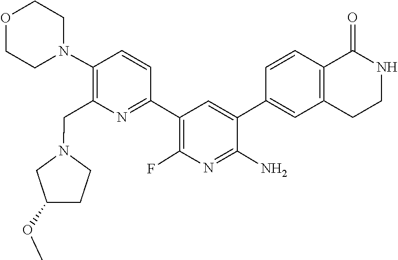 [SMILES: CO[C@H]1CCN(Cc2nc(-c3cc(-c4ccc5c(c4)CCNC5=O)c(N)nc3F)ccc2N2CCOCC2)C1] |
| I-585 | (S)-6-(6′-amino-2′-fluoro-6- ((3-methoxypyrrolidin-1- yl)methyl)-5-morpholino- [2,3′-bipyridin]-5′-yl)-7-fluoro- 3,4-dihydroisoquinolin- 1(2H)-one | 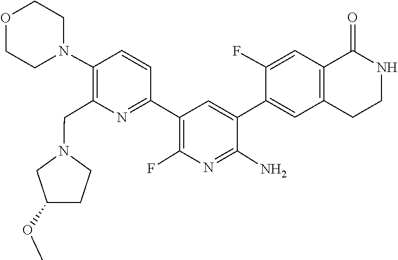 [SMILES: CO[C@H]1CCN(Cc2nc(-c3cc(-c4cc5c(cc4F)C(=O)NCC5)c(N)nc3F)ccc2N2CCOCC2)C1] |
| I-586 | 6-(2-amino-6-fluoro-5-(4- (tetrahydro-2H-pyran-4- yl)phenyl)pyridin-3-yl)-4- methylisoquinolin-1(2H)-one | 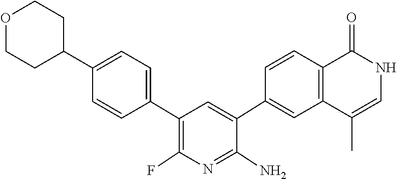 [SMILES: Cc1c[nH]c(=O)c2ccc(-c3cc(-c4ccc(C5CCOCC5)cc4)c(F)nc3N)cc12] |
| I-587 | 6-(2-amino-6-fluoro-5-(4-(4- isopropylpiperazin-1- yl)phenyl)pyridin-3-yl)-7- fluoro-3-methylisoquinolin- 1(2H)-one | 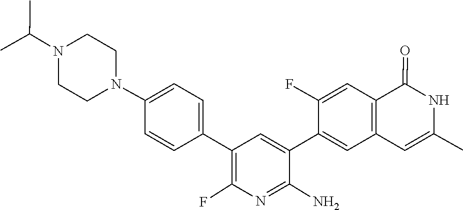 [SMILES: Cc1cc2cc(-c3cc(-c4ccc(N5CCN(C(C)C)CC5)cc4)c(F)nc3N)c(F)cc2c(=O)[nH]1] |
| I-588 | 6-(6′-amino-6- ((dimethylamino)methyl)-2′- fluoro-5-morpholino-[2,3′- bipyridin]-5′-yl)-4- methylisoquinolin-1(2H)-one | 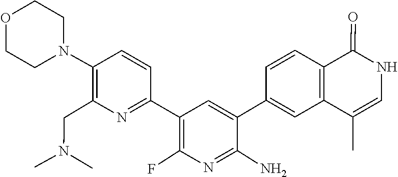 [SMILES: Cc1c[nH]c(=O)c2ccc(-c3cc(-c4ccc(N5CCOCC5)c(CN(C)C)n4)c(F)nc3N)cc12] |
| I-589 | 6-(6′-amino-6- ((dimethylamino)methyl)-2′- fluoro-5-morpholino-[2,3′- bipyridin]-5′-yl)-4- fluoroisoquinolin-1(2H)-one | 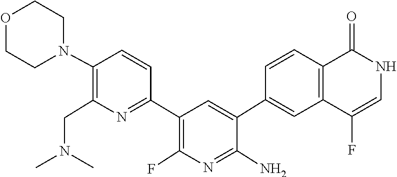 [SMILES: CN(C)Cc1nc(-c2cc(-c3ccc4c(=O)[nH]cc(F)c4c3)c(N)nc2F)ccc1N1CCOCC1] |
| I-590 | tert-butyl 4-(6′-amino-2′- fluoro-5′-(1-oxo-1,2,3,4- tetrahydroisoquinolin-6-yl)- [2,3′-bipyridin]-5-yl) pipereazine-1-carboxylate | 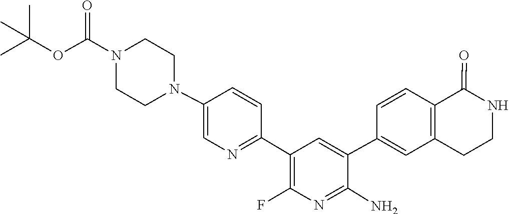 [SMILES: CC(C)(C)OC(=O)N1CCN(c2ccc(-c3cc(-c4ccc5c(c4)CCNC5=O)c(N)nc3F)nc2)CC1] |
| I-591 | 6-(5-(4-acetylpiperazin-1-yl)- 6′-amino-2′-fluoro-[2,3′- bipyridin]-5′-yl)-3,4- dihydroisoquinolin-1(2H)- one | 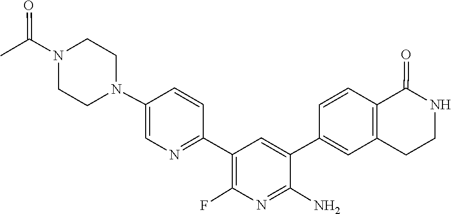 [SMILES: CC(=O)N1CCN(c2ccc(-c3cc(-c4ccc5c(c4)CCNC5=O)c(N)nc3F)nc2)CC1] |
| I-592 | 6-(5-(4-acetylpiperazin-1-yl)- 6′-amino-2′-fluoro-[2,3′- bipyridin]-5′-yl)-7-fluoro-3,4- dihydroisoquinolin-1(2H)- one | 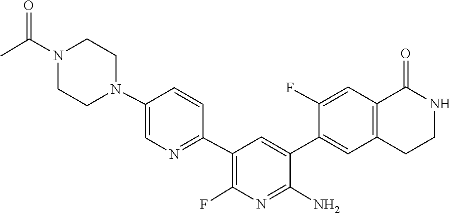 [SMILES: CC(=O)N1CCN(c2ccc(-c3cc(-c4cc5c(cc4F)C(=O)NCC5)c(N)nc3F)nc2)CC1] |
| I-593 | 6-(6′-amino-5-(4-(4,4- difluorobutyl)piperazin-1-yl)- 2′-fluoro-[2,3′-bipyridin]-5′- yl)-3,4-dihydroisoquinolin- 1(2H)-one | 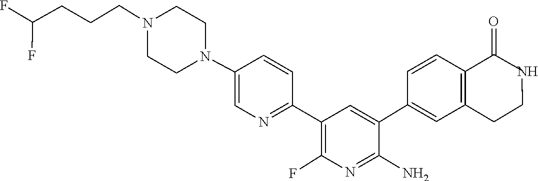 [SMILES: CC(=O)c1ccc(-c2cc(-c3ccc(N4CCN(CCCC(F)F)CC4)cn3)c(F)nc2N)cc1CCN] |
| I-594 | 6-(2-amino-5-(3- ((dimethylamino)methyl)phe- nyl)-6-fluoropyridin-3-yl)-3,4- dihydroisoquinolin-1(2H)- one | 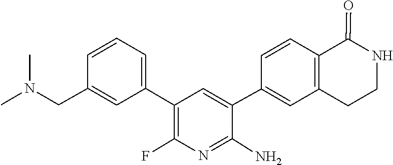 [SMILES: CN(C)Cc1cccc(-c2cc(-c3ccc4c(c3)CCNC4=O)c(N)nc2F)c1] |
| I-595 | 7-(2-amino-5-(3- ((dimethylamino)methyl)-4- ((2R,6S)-2,6- dimethylmorpholino)phenyl)- 6-fluoropyridin-3-yl)-2- methylquinazolin-4(3H)-one | 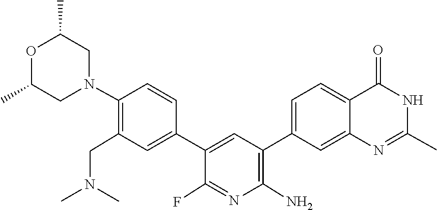 |
| I-596 | 6-(2-amino-5-(3- ((dimethylamino)methyl)-4- ((2R,6S)-2,6- dimethylmorpholino)phenyl)- 6-fluoropyridin-3-yl)-8-fluoro- 3,4-dihydroisoquinolin- 1(1H)-one | 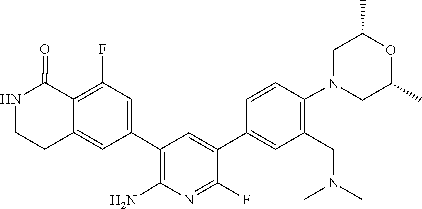 [SMILES: C[C@@H]1CN(c2ccc(-c3cc(-c4cc(F)c5c(c4)CCNC5=O)c(N)nc3F)cc2CN(C)C)C[C@H](C)O1] |
| I-597 | 6-(2-amino-5-(3- ((dimethylamino)methyl)-4- ((2R,6S)-2,6- dimethylmorpholino)phenyl)- 6-fluoropyridin-3-yl)-7-fluoro- 3,4-dihydroisoquinolin- 1(2H)-one | 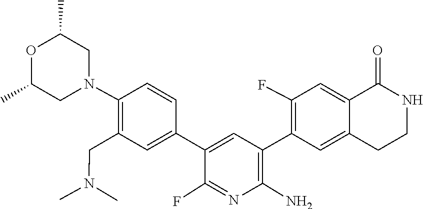 [SMILES: C[C@@H]1CN(c2ccc(-c3cc(-c4cc5c(cc4F)C(=O)NCC5)c(N)nc3F)cc2CN(C)C)C[C@H](C)O1] |
| I-598 | (S)-6-(2-amino-5-(3- (azetidin-1-ylmethyl)-4-(2- methylmorpholino)phenyl)-6- fluoropyridin-3-yl)-3,4- dihydroisoquinolin-1(2H)- one | 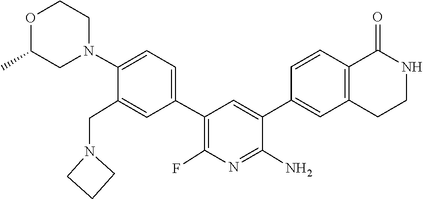 [SMILES: C[C@H]1CN(c2ccc(-c3cc(-c4ccc5c(c4)CCNC5=O)c(N)nc3F)cc2CN2CCC2)CCO1] |
| I-599 | (S)-6-(2-amino-5-(3- (azetidin-1-ylmethyl)-4-(2- methylmorpholino)phenyl)-6- fluoropyridin-3-yl)-7-fluoro- 3,4-dihydroisoquinolin- 1(2H)-one | 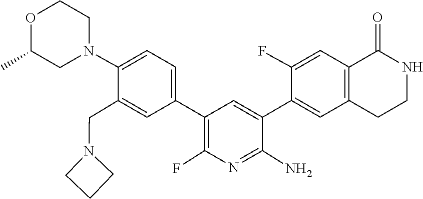 [SMILES: C[C@H]1CN(c2ccc(-c3cc(-c4cc5c(cc4F)C(=O)NCC5)c(N)nc3F)cc2CN2CCC2)CCO1] |
| I-600 | 6-(6′-amino-6- ((dimethylamino)methyl)-2′- fluoro-5-(tetrahydro-2H- pyran-4-yl)-[2,3′-bipyridin]-5′- yl)-3,4-dihydroisoquinolin- 1(2H)-one | 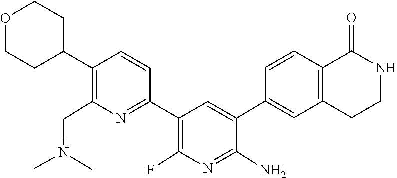 [SMILES: CN(C)Cc1nc(-c2cc(-c3ccc4c(c3)CCNC4=O)c(N)nc2F)ccc1C1CCOCC1] |
| I-601 | 6-(6′-amino-6- ((dimethylamino)methyl)-2′- fluoro-5-(tetrahydro-2H- pyran-4-yl)-[2,3′-bipyridin]-5′- yl)-7-fluoro-3,4- dihydroisoquinolin-1(2H)- one | 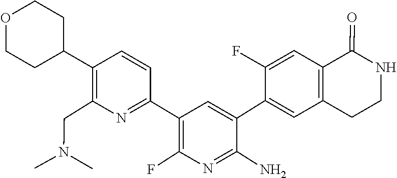 [SMILES: CN(C)Cc1nc(-c2cc(-c3cc4c(cc3F)C(=O)NCC4)c(N)nc2F)ccc1C1CCOCC1] |
| I-602 | 6-(6′-amino-6-(azetidin-1- ylmethyl)-2′-fluoro-5- (tetrahydro-2H-pyran-4-yl)- [2,3′-bipyridin]-5′-yl)-7-fluoro- 3,4-dihydroisoquinolin- 1(2H)-one | 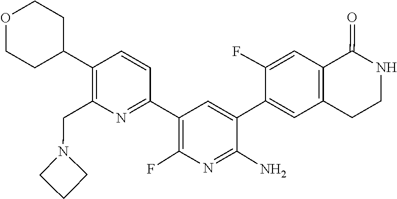 [SMILES: Nc1nc(F)c(-c2ccc(C3CCOCC3)c(CN3CCC3)n2)cc1-c1cc2c(cc1F)C(=O)NCC2] |
| I-603 | 6-(6′-amino-6- ((ethyl(methyl)amino)methyl)- 2′-fluoro-5-(tetrahydro-2H- pyran-4-yl)-[2,3′-bipyridin]-5′- yl)-3,4-dihydroisoquinolin- 1(2H)-one | 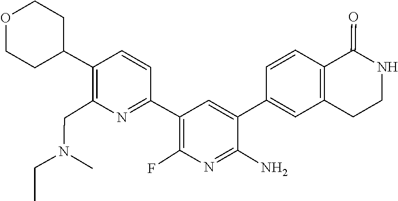 [SMILES: CCN(C)Cc1nc(-c2cc(-c3ccc4c(c3)CCNC4=O)c(N)nc2F)ccc1C1CCOCC1] |
| I-604 | 6-(6′-amino-6- ((ethyl(methyl)amino)methyl)- 2′-fluoro-5-(tetrahydro-2H- pyran-4-yl)-[2,3′-bipyridin]-5′- yl)-7-fluoro-3,4- dihydroisoquinolin-1(2H)- one | 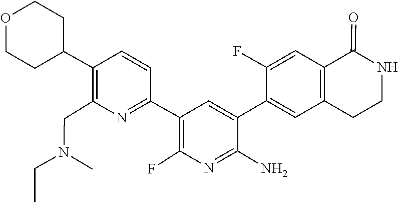 [SMILES: CCN(C)Cc1nc(-c2cc(-c3cc4c(cc3F)C(=O)NCC4)c(N)nc2F)ccc1C1CCOCC1] |
| I-605 | 6-(6′-amino-6-(azetidin-1- ylmethyl)-2′-fluoro-5- (tetrahydro-2H-pyran-4-yl)- [2,3′-bipyridin]-5′-yl)-3,4- dihydroisoquinolin-1(2H)- one | 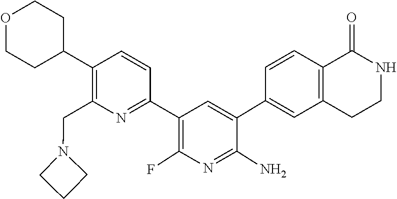 [SMILES: Nc1nc(F)c(-c2ccc(C3CCOCC3)c(CN3CCC3)n2)cc1-c1ccc2c(c1)CCNC2=O] |
| I-606 | 6-(2-amino-5-(3- ((dimethylamino)methyl)-2- fluoro-4-morpholinophenyl)- 6-fluropyridin-3-yl)-3,4- dihydroisoquinolin-1(2H)- one | 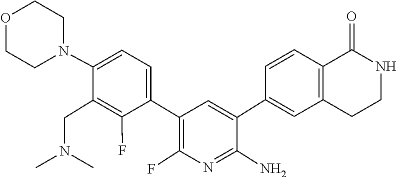 [SMILES: CN(C)Cc1c(N2CCOCC2)ccc(-c2cc(-c3ccc4c(c3)CCNC4=O)c(N)nc2F)c1F] |
| I-607 | 6-(2-amino-5-(3- ((dimethylamino)methyl)-2- fluoro-4-morpholinophenyl)- 6-fluoropyridin-3-yl)-7-fluoro- 3,4-dihydroisoquinolin- 1(2H)-one | 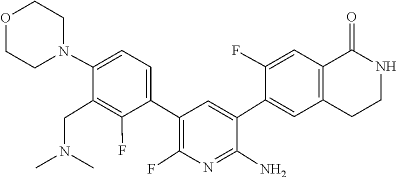 [SMILES: CN(C)Cc1c(N2CCOCC2)ccc(-c2cc(-c3cc4c(cc3F)C(=O)NCC4)c(N)nc2F)c1F] |
| I-608 | 6-(2-amino-5-(3-(azetidin-1- ylmethyl)-2-fluoro- morpholinophenyl)-6- fluoropyridin-3-yl)-7-fluoro- 3,4-dihydroisoquinolin- 1(2H)-one | 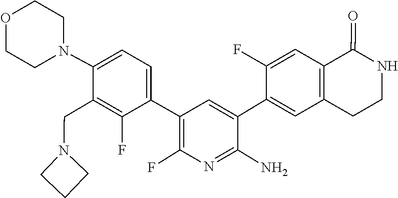 [SMILES: Nc1nc(F)c(-c2ccc(N3CCOCC3)c(CN3CCC3)c2F)cc1-c1cc2c(cc1F)C(=O)NCC2] |
| I-609 | 6-(2-amino-5-(3-(azetidin-1- ylmethyl)-2-fluoro-4- morpholinophenyl)-6- fluoropyridin-3-yl)-3,4- dihydroisoquinolin-1(2H)- one | 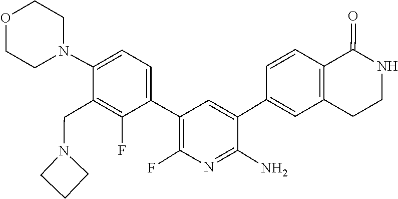 [SMILES: Nc1nc(F)c(-c2ccc(N3CCOCC3)c(CN3CCC3)c2F)cc1-c1ccc2c(c1)CCNC2=O] |
| I-610 | 6-(2-amino-5-(3- ((ethyl(methyl)amino)methyl)- 2-fluoro-4- morpholinophenyl)-6- fluoropyridin-3-yl)-3,4- dihydroisoquinolin-1(2H)- one | 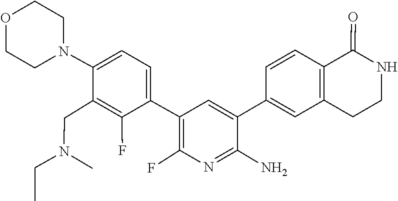 [SMILES: CCN(C)Cc1c(N2CCOCC2)ccc(-c2cc(-c3ccc4c(c3)CCNC4=O)c(N)nc2F)c1F] |
| I-611 | 6-(2-amino-5-(3- ((ethyl(methyl)amino)methyl)- 2-fluoro-4- morpholinophenyl)-6- fluoropyridin-3-yl)-7-fluoro- 3,4-dihydroisoquinolin- 1(2H)-one | 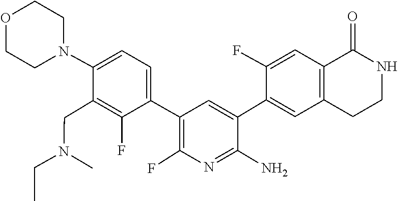 [SMILES: CCN(C)Cc1c(N2CCOCC2)ccc(-c2cc(-c3cc4c(cc3F)C(=O)NCC4)c(N)nc2F)c1F] |
| I-612 | 6-(6-amino-5′- ((dimethylamino)methyl)-2- fluoro-6′-morpholino-[3,3′- bipyridin]-5-yl)-3,4- dihydroisoquinolin-1(2H)- one | 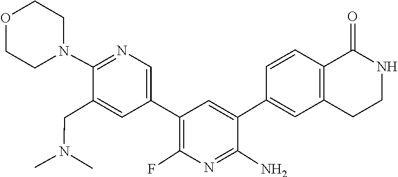 [SMILES: CN(C)Cc1cc(-c2cc(-c3ccc4c(c3)CCNC4=O)c(N)nc2F)cnc1N1CCOCC1] |
| I-613 | 6-(6′-amino-4- ((dimethylamino)methyl)-2′- fluoro-5-morpholino-[2,3′- bipyridin]-5′-yl)-3,4- dihydroisoquinolin-1(2H)- one | 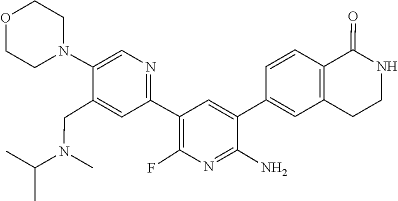 [SMILES: CC(C)N(C)Cc1cc(-c2cc(-c3ccc4c(c3)CCNC4=O)c(N)nc2F)ncc1N1CCOCC1] |
| I-614 | 6-(2-amino-6-fluoro-5-(3- ((isopropyl(methyl)amino)meth- yl)-4- morpholinoophenyl)pyridin-3- yl)-3,4-dihydroisoquinolin- 1(2H)-one | 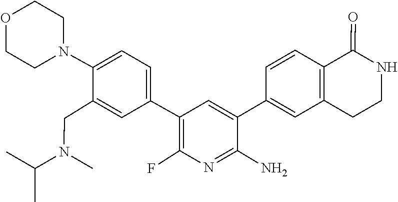 [SMILES: CC(C)N(C)Cc1cc(-c2cc(-c3ccc4c(c3)CCNC4=O)c(N)nc2F)ccc1N1CCOCC1] |
| I-615 | 6-(2-amino-5-(3- ((diethylamino)methyl)-4- morpholinophenyl)-6- fluoropyridin-3-yl)-3,4- dihydroisoquinolin-1(2H)- one | 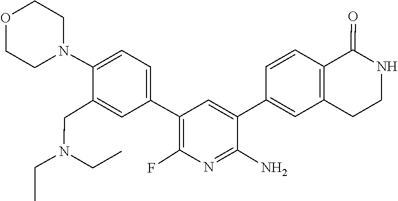 [SMILES: CCN(CC)Cc1cc(-c2cc(-c3ccc4c(c3)CCNC4=O)c(N)nc2F)ccc1N1CCOCC1] |
| I-616 | 6-(2-amino-6-fluoro-5-(3- ((methyl((tetrahydrofuran-2- yl)methyl)amino)methyl)-4- morpholinophenyl)pyridin-3- yl)-3,4-dihydroisoquinolin- 1(2H)-one | 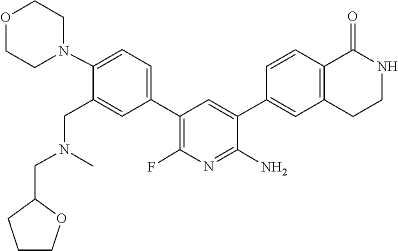 [SMILES: CN(Cc1cc(-c2cc(-c3ccc4c(c3)CCNC4=O)c(N)nc2F)ccc1N1CCOCC1)CC1CCCO1] |
| I-617 | 6-(2-amino-5-(3- ((dimethylamino)methyl)-4- fluorophenyl)-6-fluoropyridin- 3-yl)-3,4-dihydroisoquinolin- 1(2H)-one | 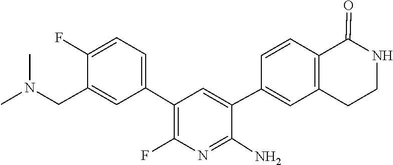 [SMILES: CN(C)Cc1cc(-c2cc(-c3ccc4c(c3)CCNC4=O)c(N)nc2F)ccc1F] |
| I-618 | 6-(2-amino-5-(3- ((dimethylamino)methyl)-4- fluorophenyl)-6-fluoropyridin- 3-yl)-7-fluoro-3,4- dihydroisoquinolin-1(2H)- one | 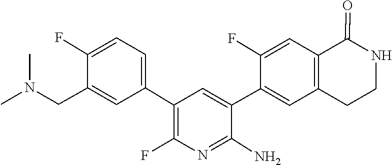 [SMILES: CN(C)Cc1cc(-c2cc(-c3cc4c(cc3F)C(=O)NCC4)c(N)nc2F)ccc1F] |
| I-619 | 6-(2-amino-6-fluoro-5-(3- ((methyl((tetrahydrofuran-2- yl)methyl)amino)methyl)-4- morpholinophenyl)pyridin-3- yl)-7-fluoro-3,4- dihydroisoquinolin-1(2H)- one | 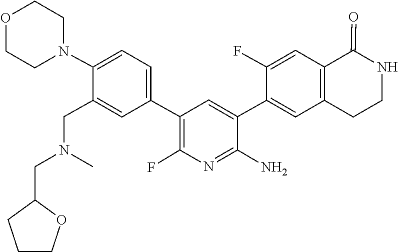 [SMILES: CN(Cc1cc(-c2cc(-c3cc4c(cc3F)C(=O)NCC4)c(N)nc2F)ccc1N1CCOCC1)CC1CCCO1] |
| I-620 | 6-(2-amino-5-(3- ((dimethylamino)methyl)-4- (tetrahydrofuran-3- yl)phenyl)-6-fluoropyridin-3- yl)-3,4-dihydroisoquinolin- 1(2H)-one | 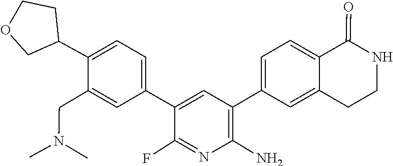 [SMILES: CN(C)Cc1cc(-c2cc(-c3ccc4c(c3)CCNC4=O)c(N)nc2F)ccc1C1CCOC1] |
| I-621 | 6-(2-amino-5-(3- ((dimethylamino)methyl)-4- (tetrahydrofuran-3- yl)phenyl)-6-fluoropyridin-3- yl)-7-fluoro-3,4- dihydroisoquinolin-1(2H)- one | 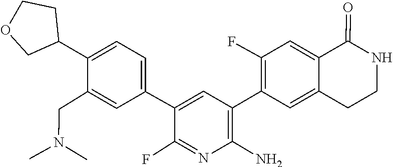 [SMILES: CN(C)Cc1cc(-c2cc(-c3cc4c(cc3F)C(=O)NCC4)c(N)nc2F)ccc1C1CCOC1] |
| I-622 | 6-(2-amino-5-(5- ((dimethylamino)methyl)-2- fluoro-4-morpholinophenyl)- 6-fluoropyridin-3-yl)-3,4- dihydroisoquinolin-1(2H)- one | 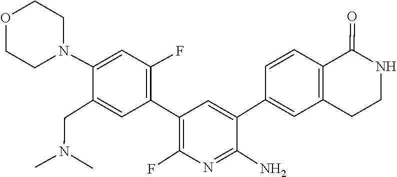 [SMILES: CN(C)Cc1cc(-c2cc(-c3ccc4c(c3)CCNC4=O)c(N)nc2F)c(F)cc1N1CCOCC1] |
| I-623 | 6-(2-amino-5-(5- ((dimethylamino)methyl)-2,3- difluoro-4- morpholinophenyl)-6- fluoropyridin-3-yl)-3,4- dihydroisoquinolin-1(2H)- one | 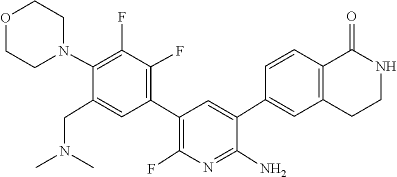 [SMILES: CN(C)Cc1cc(-c2cc(-c3ccc4c(c3)CCNC4=O)c(N)nc2F)c(F)c(F)c1N1CCOCC1] |
| I-624 | 6-(2-amino-5-(3-(1- (dimethylamino)ethyl)-4- hydroxyphenyl)-6- fluoropyridin-3-yl)-3,4- dihydroisoquinolin-1(2H)- one | 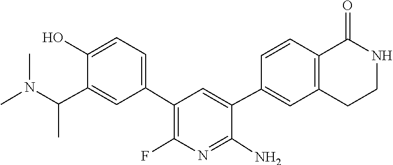 [SMILES: CC(c1cc(-c2cc(-c3ccc4c(c3)CCNC4=O)c(N)nc2F)ccc1O)N(C)C] |
| I-625 | 6-(2-amino-5-(3-(2- aminopropan-2-yl)-4- fluorophenyl)-6-fluoropyridin- 3-yl)-3,4-dihydroisoquinolin- 1(2H)-one | 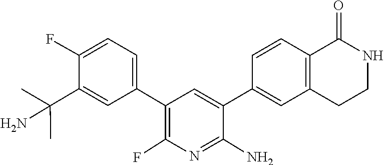 [SMILES: CC(C)(N)c1cc(-c2cc(-c3ccc4c(c3)CCNC4=O)c(N)nc2F)ccc1F] |
| I-626 | 6-(2-amino-5-(3- ((dimethylamino)methyl)-4- fluorophenyl)-6-fluoropyridin- 3-yl)-4-methylisoquinolin- 1(2H)-one | 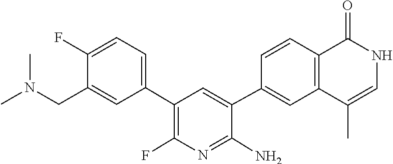 [SMILES: Cc1c[nH]c(=O)c2ccc(-c3cc(-c4ccc(F)c(CN(C)C)c4)c(F)nc3N)cc12] |
| I-627 | 6-(2-amino-5-(3- ((dimethylamino)methyl)phe- nyl)-6-fluoropyridin-3-yl)-4- methylisoquinolin-1(2H)-one | 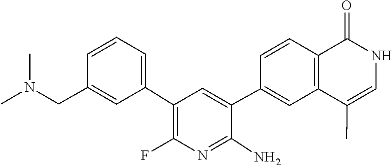 [SMILES: Cc1c[nH]c(=O)c2ccc(-c3cc(-c4cccc(CN(C)C)c4)c(F)nc3N)cc12] |
| I-628 | (R)-6-(2-amnio-5-(4- (dimethylamino)chroman-6- yl)-6-fluoropyridin-3-yl)-4- methylisoquinolin-1(2H)-one | 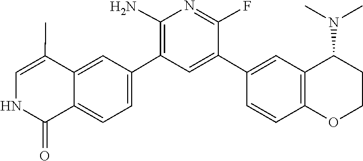 [SMILES: Cc1c[nH]c(=O)c2ccc(-c3cc(-c4ccc5c(c4)[C@H](N(C)C)CCO5)c(F)nc3N)cc12] |
| I-629 | (S)-6-(2-amino-5-(3- (dimethylamino)-2,3- dihydrobenzofuran-5-yl)-6- fluoropyridin-3-yl)-4- methylisoquinolin-1(2H)-one | 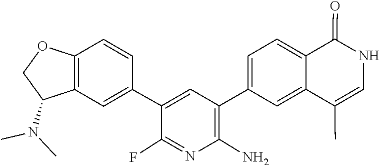 [SMILES: Cc1c[nH]c(=O)c2ccc(-c3cc(-c4ccc5c(c4)[C@H](N(C)C)CO5)c(F)nc3N)cc12] |
| I-630 | (S)-6-(2-amino-5-(3- (dimethylamino)-2,3- dihydrobenzofuran-5-yl)-6- fluoropyridin-3-yl)-4- fluoroisoquinolin-1(2H)-one | 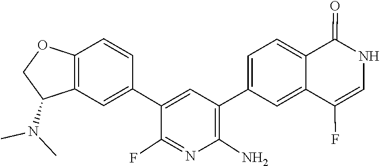 [SMILES: CN(C)[C@@H]1COc2ccc(-c3cc(-c4ccc5c(=O)[nH]cc(F)c5c4)c(N)nc3F)cc21] |
| I-631 | (R)-6-(2-amino-5-(4- (dimethylamino)chroman-6- yl)-6-fluoropyridin-3-yl)-4- fluoroisoquinolin-1(2H)-one | 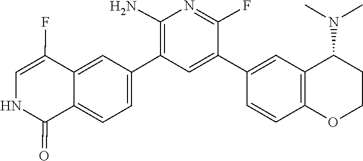 [SMILES: CN(C)[C@@H]1CCOc2ccc(-c3cc(-c4ccc5c(=O)[nH]cc(F)c5c4)c(N)nc3F)cc21] |
| I-632 | 5-(6-amino-2-fluoro-5-(1- oxo-1,2,3,4- tetrahydroisoquinolin-6- yl)pyridin-3-yl)-2- methoxybenzonitrile | 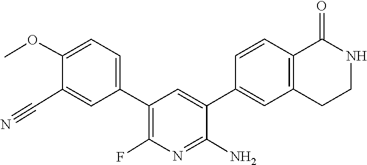 [SMILES: COc1ccc(-c2cc(-c3ccc4c(c3)CCNC4=O)c(N)nc2F)cc1C#N] |
| I-633 | 6-(2-amino-5-(3- ((ethyl(methyl)amino)methyl)- 4-methoxyphenyl)-6- fluoropyridin-3-yl)-4- fluoroisoquinolin-1(2H)-one | 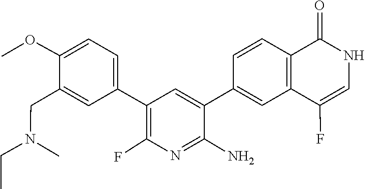 [SMILES: CCN(C)Cc1cc(-c2cc(-c3ccc4c(=O)[nH]cc(F)c4c3)c(N)nc2F)ccc1OC] |
| I-634 | 6-(2-amino-5-(3- ((ethyl(methyl)amino)methyl)- 4-methoxyphenyl)-6- fluoropyridin-3-yl)-4- methylisoquinolin-1(2H)-one | 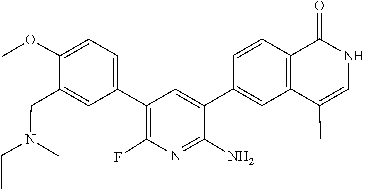 [SMILES: CCN(C)Cc1cc(-c2cc(-c3ccc4c(=O)[nH]cc(C)c4c3)c(N)nc2F)ccc1OC] |
| I-635 | 8-(2-amino-5-(4-(4- (cyclopropylmethyl)piperazin- 1-yl)phenyl)-6-fluoropyridin- 3-yl)-6-fluoro-3,4- dihydrobenzo[f][1,4]oxazepin- 5(2H)-one | 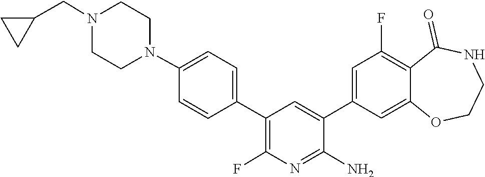 [SMILES: Nc1nc(F)c(-c2ccc(N3CCN(CC4CC4)CC3)cc2)cc1-c1cc(F)c2c(c1)OCCNC2=O] |
| I-636 | 8-(2-amino-6-fluoro-5-(4-(4- isopropylpiperazin-1- yl)phenyl)pyridin-3-yl)-6- fluoro-3,4- dihydrobenzo[f][1,4]oxazepin- 5(2H)-one | 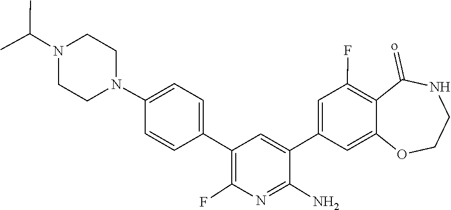 |
| I-637 | 5-(2-amino-6-fluoro-5-(4-(4- isopropylpiperazin-1- yl)phenyl)pyridin-3-yl)-7- fluoroisoindolin-1-one | 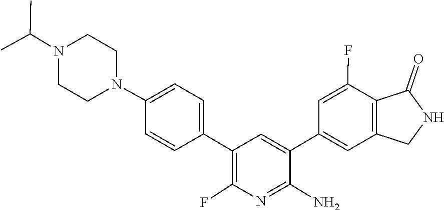 [SMILES: CC(C)N1CCN(c2ccc(-c3cc(-c4cc(F)c5c(c4)CNC5=O)c(N)nc3F)cc2)CC1] |
| I-638 | 6-(2-amino-6-fluoro-5-(3-(3- ((4-methyl-4H-1,2,4-triazol- 3-yl)methyl)oxetan-3- yl)phenyl)pyridin-3-yl)-3,4- dihydroisoquinolin-1(2H)- one | 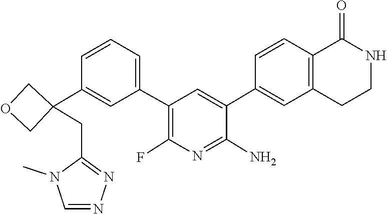 [SMILES: Cn1cnnc1CC1(c2cccc(-c3cc(-c4ccc5c(c4)CCNC5=O)c(N)nc3F)c2)COC1] |
| I-639 | 6-((6-fluoro-5-(4-(4- isoproylpiperazin-1- yl)phenyl)pyridin-2- yl)amino)-3,4- dihydroisoquinolin-1(2H)- one | 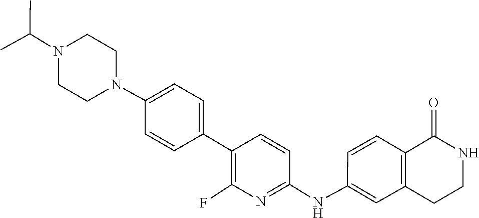 [SMILES: CC(C)N1CCN(c2ccc(-c3ccc(Nc4ccc5c(c4)CCNC5=O)nc3F)cc2)CC1] |
| I-640 | 6-(2-amino-5-(4-(4-((2,2- difluorocyclopropyl)methyl)pi- perazin-1-yl)phenyl)-6- fluoropyridin-3-yl)-3,4- dihydroisoquinolin-1(2H)- one | 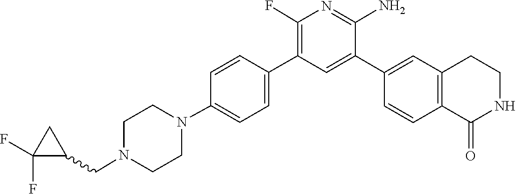 [SMILES: Nc1nc(F)c(-c2ccc(N3CCN(CC4CC4(F)F)CC3)cc2)cc1-c1ccc2c(c1)CCNC2=O] |
| I-641 | 6-(2-amino-5-(4-(4-(4,4- difluorobutyl)piperazin-1- yl)phenyl)-6-fluoropyridin-3- yl)-3,4-dihydroisoquinolin- 1(2H)-one | 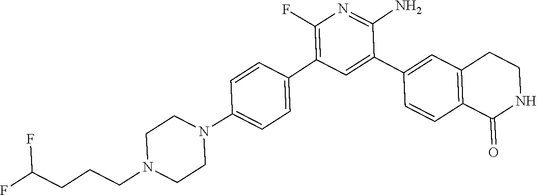 [SMILES: Nc1nc(F)c(-c2ccc(N3CCN(CCCC(F)F)CC3)cc2)cc1-c1ccc2c(c1)CCNC2=O] |
| I-642 | (S)-6-(2-amino-6-fluoro-5-(3- (1-methylpyrrolidin-2-yl)-4- (tetrahydro-2H-pyran-4- yl)phenyl)pyridin-3-yl)-3,4- dihydroisoquinolin-1(2H)- one | 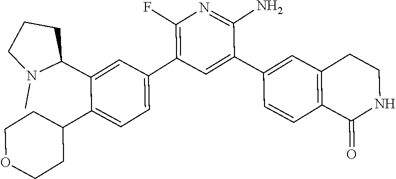 [SMILES: CN1CCC[C@H]1c1cc(-c2cc(-c3ccc4c(c3)CCNC4=O)c(N)nc2F)ccc1C1CCOCC1] |
| I-643 | (S)-7-(2-amino-6-fluoro-5-(3- (pyrrolidin-2-yl)-4- (tetrahydro-2H-pyran-4- yl)phenyl)pyridin-3-yl)-2- methylquinazolin-4(3H)-one | 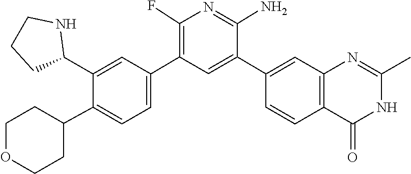 [SMILES: Cc1nc2cc(-c3cc(-c4ccc(C5CCOCC5)c([C@@H]5CCCN5)c4)c(F)nc3N)ccc2c(=O)[nH]1] |
| I-644 | 6-(2-amino-5-(4-(4-(2-(2,2- difluorocyclopropyl)ethyl)piper- azin-1-yl)phenyl)-6- fluoropyridin-3-yl)-3,4- dihydroisoquinolin-1(2H)- one | 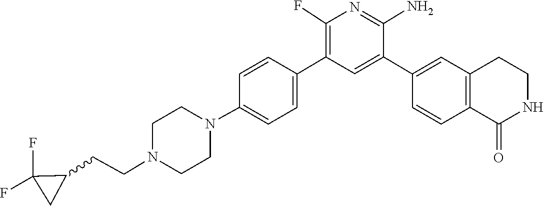 [SMILES: Nc1nc(F)c(-c2ccc(N3CCN(CCC4CC4(F)F)CC3)cc2)cc1-c1ccc2c(c1)CCNC2=O] |
| I-645 | 6-(2-amino-5-(3- ((dimethylamino)methyl)-4- (tetrahydro-2H-pyran-4- yl)phenyl)-6-fluoropyridin-3- y)-3,4-dihydroisoquinolin- 1(2H)-one | 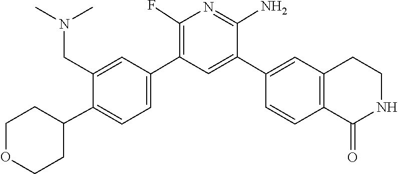 [SMILES: CN(C)Cc1cc(-c2cc(-c3ccc4c(c3)CCNC4=O)c(N)nc2F)ccc1C1CCOCC1] |
| | | and |
| I-646 | (S)-6-(2-amino-6-fluoro-5-(3- (pyrrolidin-2- yl)phenyl)pyridin-3-yl)-3,4- dihydroisoquinolin-1(2H)- one | 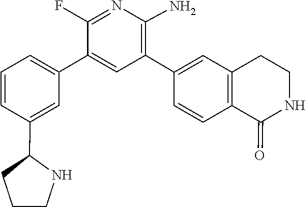 [SMILES: Nc1nc(F)c(-c2cccc([C@@H]3CCCN3)c2)cc1-c1ccc2c(c1)CCNC2=O] |

or a pharmaceutically acceptable salt, solvate and/or prodrug thereof.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds of the present application may also exist in different tautomeric forms and it is intended that any tautomeric forms which the compounds form, as well as mixtures thereof, are included within the scope of the present application.

The compounds of the present application may further exist in varying polymorphic forms and it is contemplated that any polymorphs, or mixtures thereof, which form are included within the scope of the present application.

The compounds of the present application also include compounds having alternate isotopes, including radioactive and non-radioactive isotopes, for any of the atoms. For example, in an embodiment, the compounds of the application include compounds wherein one or more available hydrogen atoms have been substituted with deuterium. in an embodiment, the compounds of the application include compounds wherein one or more available carbon atoms have been substituted with ¹³C.

In an embodiment the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of compounds of the application include, for example, those made with solvents that are pharmaceutically acceptable. Examples of such solvents include water (resulting solvate is called a hydrate) and ethanol and the like.

Prodrugs of the compounds of the present application may be, for example, conventional esters formed with available hydroxy, thiol, amino or carboxyl groups. Some common esters which have been utilized as prodrugs are phenyl esters, aliphatic (C₁C₂₄) esters, acyloxymethyl esters, carbamates and amino acid esters.

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier.

A compound of the application including salts and/or solvates thereof is suitably used on their own but will generally be administered in the form of a composition in which the one or more compounds of the application (the active ingredient) is in association with an acceptable carrier. Depending on the mode of administration, the composition will comprise from about 0.05 wt % to about 99 wt % or about 0.10 wt % to about 70 wt %, of the active ingredient, and from about 1 wt % to about 99.95 wt % or about 30 wt % to about 99.90 wt % of an acceptable carrier, all percentages by weight being based on the total composition.

The compounds of the application may be administered to a subject in a variety of forms depending on the selected route of administration, as will be understood by those skilled in the art. A compound of the application may be administered, for example, by oral, parenteral, buccal, sublingual, nasal, rectal, patch, pump or transdermal administration and the pharmaceutical compositions formulated accordingly. Administration can be by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists.

A compound of the application may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound may be incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. Liposomes can be formed from a variety of phospholipids, such as cholesterol, stearylamine or phosphatidylcholines. For oral administration in a capsule form, useful carriers or diluents include lactose and dried corn starch.

Liquid preparations for oral administration may take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents may be added. Such liquid preparations for oral administration may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

A compound of the application may also be administered parenterally. Solutions of a compound of the application can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

The compounds of the application may be formulated for parenteral administration by injection, including using conventional catheterization techniques or infusion. Formulations for injection may be presented in unit dosage form, e.g., in ampoules or in multi-dose containers, with an added preservative. The compositions may take such forms as sterile suspensions, solutions or emulsions in oily or aqueous vehicles, and may contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

Compositions for nasal administration may conveniently be formulated as aerosols, drops, gels and powders.

For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, P A, 1980, pp. 1530-1533 for further discussion of suppository dosage forms.

Compounds of the application may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, compounds of the application may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In an embodiment, compounds of the application may be coupled with viral, non-viral or other vectors. Viral vectors may include retrovirus, lentivirus, adenovirus, herpesvirus, poxvirus, alphavirus, vaccinia virus or adeno-associated viruses. Non-viral vectors may include nanoparticles, cationic lipids, cationic polymers, metallic nanoparticles, nanorods, liposomes, micelles, microbubbles, cell-penetrating peptides, or lipospheres. Nanoparticles may include silica, lipid, carbohydrate, or other pharmaceutically acceptable polymers.

In some embodiments, depending on the mode of administration, the pharmaceutical composition will comprise from about 0.05 wt % to about 99 wt % or about 0.10 wt % to about 70 wt %, of the active ingredient (one or more compounds of the application), and from about 1 wt % to about 99.95 wt % or about 30 wt % to about 99.90 wt % of one or more pharmaceutically acceptable carriers, all percentages by weight being based on the total composition.

In an embodiment, a compound of the present application is administered with another therapeutic agent simultaneously or sequentially in separate unit dosage forms or together in a single unit dosage form. Accordingly, the present application provides a single unit dosage form comprising one or more compounds of the application (e.g. a compound of Formula (I)), an additional therapeutic agent, and a pharmaceutically acceptable carrier.

To be clear, in the above, the term “a compound” also includes embodiments wherein one or more compounds are referenced.

### III. Methods and Uses of the Application

The compounds of the application have been shown to be capable of inhibiting HPK1 activity. In an embodiment, the HPK1 is human HPK1, see for example, Hu, M. C. et. al.; Genes Dev. 10 (1): 2251-2264, 1996.

Accordingly, the present application includes a method for inhibiting HPK1, in a cell, either in a biological sample or in a patient, comprising administering an effective amount of one or more compounds of the application to the cell. The application also includes a use of one or more compounds of the application for inhibiting HPK1 in a cell as well as a use of one or more compounds of the application for the preparation of a medicament for inhibiting HPK1 in a cell. The application further includes one or more compounds of the application for use in inhibiting HPK1.

As the compounds of the application have been shown to be capable of inhibiting HPK1, the compounds of the application are useful for treating diseases, disorders or conditions by inhibiting HPK1. Therefore the compounds of the present application are useful as medicaments. Accordingly, the present application includes a compound of the application for use as a medicament.

The present application also includes a method of treating a disease, disorder or condition that is treatable by inhibiting HPK1 comprising administering a therapeutically effective amount of one or more compounds of the application to a subject in need thereof.

The present application also includes a use of one or more compounds of the application for treatment of a disease, disorder or condition that is treatable by inhibiting HPK1 as well as a use of one or more compounds of the application for the preparation of a medicament for treatment of a disease, disorder or condition that is treatable by inhibiting HPK1. The application further includes one or more compounds of the application for use in treating a disease, disorder or condition that is treatable by inhibiting HPK1.

In an embodiment, the disease, disorder or condition that is treatable by inhibiting HPK1 is a neoplastic disorder. Accordingly, the present application also includes a method of treating a neoplastic disorder comprising administering a therapeutically effective amount of one or more compounds of the application to a subject in need thereof. The present application also includes a use of one or more compounds of the application for treatment of a neoplastic disorder as well as a use of one or more compounds of the application for the preparation of a medicament for treatment of a neoplastic disorder. The application further includes one or more compounds of the application for use in treating a neoplastic disorder. In an embodiment, the treatment is in an amount effective to ameliorate at least one symptom of the neoplastic disorder, for example, reduced cell proliferation or reduced tumor mass, among others, in a subject in need of such treatment.

Compounds of the application have been demonstrated to inhibit HPK1 and hence cytokine release in immune derived cell (e.g. Jurkat-T cells). Therefore in another embodiment of the present application, the disease, disorder or condition that is treatable by inhibiting HPK1 is cancer. Accordingly, the present application also includes a method of treating cancer comprising administering a therapeutically effective amount of one or more compounds of the application to a subject in need thereof. The present application also includes a use of one or more compounds of the application for treatment of cancer as well as a use of one or more compounds of the application for the preparation of a medicament for treatment of cancer. The application further includes one or more compounds of the application for use in treating cancer. In an embodiment, the compound is administered for the prevention of cancer in a subject such as a mammal having a predisposition for cancer.

In some embodiments, the cancer is selected from, but not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated in accordance with the methods described herein.

In an embodiment, the cancer is selected from hematologic cancers, breast cancers, ovarian cancers, lung cancers, melanomas, colon cancers and glioblastomas.

In an embodiment, the disease, disorder or condition that is treatable by inhibiting HPK1 is a disease, disorder or condition associated with an uncontrolled and/or abnormal cellular activity affected directly or indirectly by inhibiting HPK1. In another embodiment, the uncontrolled and/or abnormal cellular activity that is affected directly or indirectly by inhibiting HPK1 is proliferative activity in a cell. Accordingly, the application also includes a method of inhibiting proliferative activity in a cell, comprising administering an effective amount of one or more compounds of the application to the cell. The present application also includes a use of one or more compounds of the application for inhibition of proliferative activity in a cell as well as a use of one or more compounds of the application for the preparation of a medicament for inhibition of proliferative activity in a cell. The application further includes one or more compounds of the application for use in inhibiting proliferative activity in a cell by boosting immune cell function through HPK1 inhibition.

The present application also includes a method of inhibiting uncontrolled and/or abnormal cellular activities affected directly or indirectly by inhibiting HPK1 in a cell, either in a biological sample or in a subject, comprising administering an effective amount of one or more compounds of the application to the cell. The application also includes a use of one or more compounds of the application for inhibition of uncontrolled and/or abnormal cellular activities affected directly or indirectly by inhibiting HPK1 in a cell as well as a use of one or more compounds of the application for the preparation of a medicament for inhibition of uncontrolled and/or abnormal cellular activities affected directly or indirectly by inhibiting HPK1 in a cell. The application further includes one or more compounds of the application for use in inhibiting uncontrolled and/or abnormal cellular activities affected directly or indirectly by inhibiting HPK1 in a cell.

The present application also includes a method of treating a disease, disorder or condition that is treatable by inhibiting HPK1 comprising administering a therapeutically effective amount of one or more compounds of the application in combination with another known agent useful for treatment of a disease, disorder or condition that is treatable by inhibiting HPK1 to a subject in need thereof. The present application also includes a use of one or more compounds of the application in combination with another known agent useful for treatment of a disease, disorder or condition that is treatable by inhibiting HPK1 for treatment of a disease, disorder or condition that is treatable by inhibiting HPK1, as well as a use of one or more compounds of the application in combination with another known agent useful for treatment of a disease, disorder or condition that is treatable by inhibiting HPK1 for the preparation of a medicament for treatment of a disease, disorder or condition that is treatable by inhibiting HPK1. The application further includes one or more compounds of the application in combination with another known agent useful for treatment of a disease, disorder or condition that is treatable by inhibiting HPK1 for use in treating a disease, disorder or condition that is treatable by inhibiting HPK1. In an embodiment, the disease, disorder or condition treatable by inhibiting HPK1 is cancer.

In a further embodiment, the disease, disorder or condition that is treatable by inhibiting HPK1 is cancer and the one or more compounds of the application are administered in combination with one or more additional cancer treatments. In another embodiment, the additional cancer treatment is selected from radiotherapy, chemotherapy, targeted therapies such as antibody therapies (including anti-PD1 and/or anti-PD-L1 antibodies) and small molecule therapies such as tyrosine-kinase inhibitors and glutaminase inhibitors (GLS1 inhibitors), immunotherapy, hormonal therapy and anti-angiogenic therapies.

When used in combination with other agents or therapies useful in treating diseases, disorders or conditions that are treatable by inhibiting HPK1, it is an embodiment that the compounds of the application are administered contemporaneously with those agents or therapies. As used herein, “contemporaneous administration” of two substances or therapies to a subject means providing each of the two substances or therapies so that they are both biologically active in the individual at the same time. The exact details of the administration will depend on the pharmacokinetics of the two substances or therapies in the presence of each other, and can include administering the two substances or therapies within a few hours of each other, or even administering one substance or therapy within 24 hours of administration of the other, if the pharmacokinetics are suitable. Design of suitable dosing regimens is routine for one skilled in the art. In particular embodiments, the substances or therapies will be administered substantially simultaneously, i.e., within minutes of each other, or in a single composition in the case of administration of two substances. It is a further embodiment of the present application that a combination of agents or therapies is administered to a subject in a non-contemporaneous fashion.

In an embodiment, the subject is a mammal. In an embodiment, the subject is human.

In the context of treating a disease, disorder or condition treatable by inhibition of HPK1, an effective amount is an amount that, for example, inhibits HPK1, compared to the inhibition without administration of the one or more compounds. Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art. The effective amount is one that following treatment therewith manifests as an improvement in or reduction of any disease symptom. When the disease is cancer, amounts that are effective can cause a reduction in the number, growth rate, size and/or distribution of tumours.

The dosage of compounds of the application can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. Compounds of the application may be administered initially in a suitable dosage that may be adjusted as required, depending on the clinical response. Dosages will generally be selected to maintain a serum level of compounds of the application from about 0.01 μg/cc to about 1000 μg/cc, or about 0.1 μg/cc to about 100 μg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg. In an embodiment of the application, compositions are formulated for oral administration and the compounds are suitably in the form of tablets containing 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per tablet. Compounds of the application may be administered in a single daily, weekly or monthly dose or the total daily dose may be divided into two, three or four daily doses.

Treatment methods comprise administering to a subject a therapeutically effective amount of one or more of the compounds of the application and optionally consist of a single administration, or alternatively comprise a series of administrations, and optionally comprise concurrent administration or use of one or more other therapeutic agents. In an embodiment, the compounds of the application are administered at least once a week. However, in another embodiment, the compounds are administered to the subject from about one time per two weeks, three weeks or one month. In another embodiment, the compounds are administered about one time per week to about once daily. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

### IV. Methods of Preparing the Compounds of the Application

Compounds of the present application can be prepared by various synthetic processes. The choice of particular structural features and/or substituents may influence the selection of one process over another. The selection of a particular process to prepare a given compound of Formula (I) is within the purview of the person of skill in the art. Some starting materials for preparing compounds of the present application are available from commercial chemical sources. Other starting materials, for example as described below, are readily prepared from available precursors using straightforward transformations that are well known in the art. In the Schemes below showing the preparation of compounds of the application, all variables are as defined in Formula I, unless otherwise stated.

The compounds of Formula (I) generally can be prepared according to the processes illustrated in the Schemes below. In the structural formulae shown below the variables are as defined in Formula (I) unless otherwise stated. A person skilled in the art would appreciate that many of the reactions depicted in the Schemes below would be sensitive to oxygen and water and would know to perform the reaction under an anhydrous, inert atmosphere if needed. Reaction temperatures and times are presented for illustrative purposes only and may be varied to optimize yield as would be understood by a person skilled in the art.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999).

It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994).

Techniques for purification of intermediates and final products include, for example, straight and reversed phase chromatography on column or rotating plate, recrystallisation, distillation and liquid-liquid or solid-liquid extraction, which will be readily understood by one skilled in the art.

The products of the processes of the application may be isolated according to known methods, for example, the compounds may be isolated by evaporation of the solvent, by filtration, centrifugation, chromatography or other suitable method.

Generally, the reactions described above are performed in a suitable inert organic solvent and at temperatures and for times that will optimize the yield of the desired compounds. Examples of suitable inert organic solvents include, but are not limited to, 2-propanol, dimethylformamide (DMF), 1,4-dioxane, methylene chloride, chloroform, tetrahydrofuran (THF), toluene, and the like.

The formation of a desired compound salt is achieved using standard techniques. For example, the neutral compound is treated with an acid or base in a suitable solvent and the formed salt is isolated by filtration, extraction or any other suitable method.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Prodrugs of the compounds of the present application may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine).

One skilled in the art will recognize that where a reaction step of the present application is carried out in a variety of solvents or solvent systems, said reaction step may also be carried out in a mixture of the suitable solvents or solvent systems.

Accordingly, in an embodiment, the compounds of Formula (I) are prepared as shown in Scheme 1.

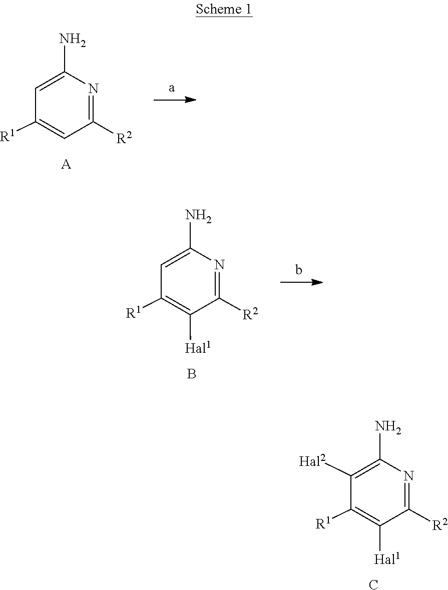

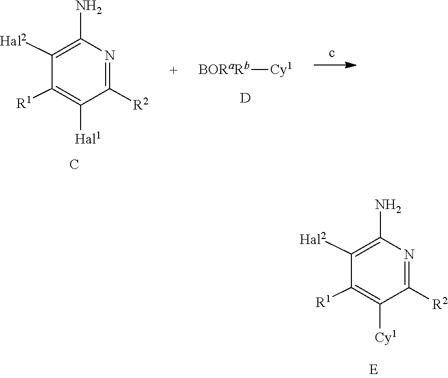

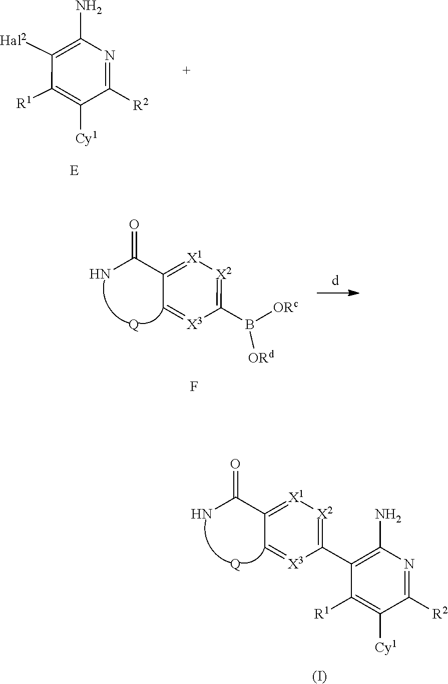

Accordingly, aminopyridine compounds of Formula (A) are mono-halogenated to form compounds of Formula (B), followed by subsequent halogenation to form intermediate compounds of Formula (C) wherein Hal¹ and Hal² are independently a halogen. Intermediate (C) is then coupled to boronic acid or boronic ester intermediate compounds of Formula (D) to form aminopyridine intermediate compounds of Formula (E). In an embodiment Hal¹ and Hal² are different halogens selected to have differing reactivity in the coupling reactions as would be known to those skilled in the art. In an embodiment Hal¹ and Hal² are Br and Cl respectively. In an embodiment Hal¹ and Hal² are I and Cl respectively. In an embodiment Hal¹ and Hal² are I and Br respectively. In an embodiment, R^{a} and R^{b} or R^{c} and R^{d} are all H. In an embodiment, R^{a} and R^{b} or R^{c} and R^{d} form a cycloalkyl ring. Compounds of Formula (E) are then coupled to boronic acid or ester compounds of Formula (F) to form the compounds of Formula (I). The variables R¹, R², Q, X¹, X², X³, Cy¹ are as defined in Formula (I). In an embodiment, both coupling reactions are performed under cross-coupling conditions, such as in the presence of a cross-coupling catalyst and in an inert solvent. In some embodiments the cross-coupling catalyst is a palladium catalyst. In an embodiment the halogenation conditions comprise a halogenation reagent, such as N-bromosuccinamide.

In a further embodiment, the compounds of Formula (I) are synthesized as shown in Scheme 2 by first coupling a boronic acid or a boronic ester compound of Formula (F) with a dihalogenated intermediate compound of Formula (G) by a Suzuki-Miyaura reaction to form the intermediate compound of Formula (H). Intermediate compound of Formula (H) is then reacted with a suitable boronic acid or boronic ester intermediate compound of Formula (D) to form a compound of Formula (I). The variables R¹, R², Q, X¹, X², X³, Cy¹ are as defined in Formula (I). In an embodiment Hal¹ and Hal² are different halogens selected to have differing reactivity in the coupling reactions as would be known to those skilled in the art. In an embodiment of the application, Hal¹ and Hal² are Br and I respectively. In an embodiment, R^{a} and R^{b} or R^{c} and R^{d} are all H. In an embodiment, R^{a} and R^{b} or R^{c} and R^{d} together form a cycloalkyl ring. In an embodiment, both coupling reactions are performed under cross-coupling conditions, such as in the presence of a cross-coupling catalyst and in an inert solvent. In some embodiments the cross-coupling catalyst is a palladium catalyst.

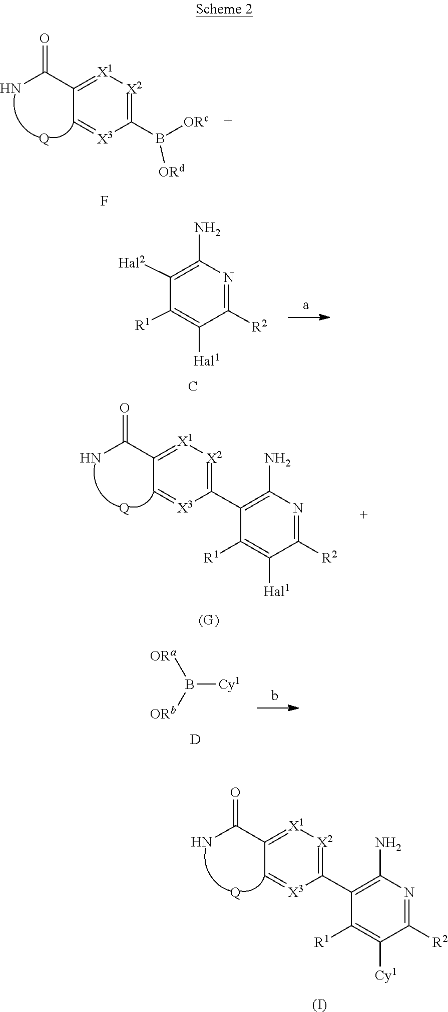

In a further embodiment, the compounds of Formula (C) are synthesized as shown in Scheme 3. Therefore, compounds of Formula (C) can be prepared by treating intermediate compound of Formula (A) with a halogenating reagent such as NBS to form mono-halogenated intermediate compound of Formula B which is then treated with a halogenating reagent such as N-iodosuccinimide (NIS) to provide the compounds of Formula C. In an embodiment Hal¹ and Hal² are different halogens selected to have differing reactivity in the coupling reactions as would be known to those skilled in the art. In an embodiment, Hal¹ and Hal² are, for example, Br and I respectively.

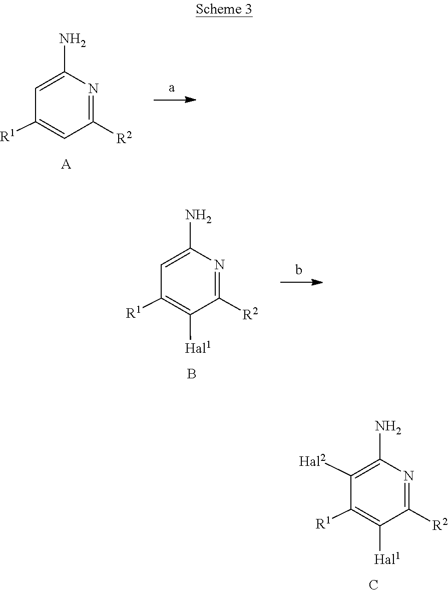

In an embodiment, the compounds of Formula (F) are synthesized as shown in Scheme 4. Therefore, compounds of Formula (F) are prepared by sequentially treating intermediate compounds of Formula (H) wherein Hal is a halogen with a suitable borylating agent such as 4,4,4′,4′,5,5,5′,5′-octamethyl-2,2′-bi(1,3,2-dioxaborolane in the presence of a suitable catalyst such as [1,1′-bis(diphenylphosphino)ferrocene]dichloropalladium(II) dichloromethane in an inert solvent such as 1,4-dixoane.

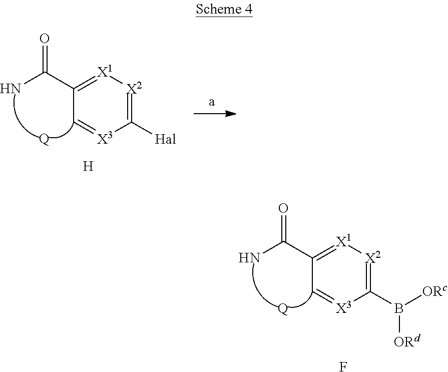

In an embodiment, as shown in Scheme 5, compounds of Formula (I) wherein Cy¹ is phenyl, and R⁹ is

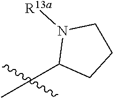

wherein R^{13a} is H or R¹³ are prepared by coupling a boronic acid or boronic ester compound of Formula (J) with a halogenated compound of Formula (K) under suitable conditions, for example, under Suzuki coupling conditions, to form compounds of Formula (I). In an embodiment, R⁹ is an amino protecting group, for example, tert-butyloxycarbonyl (Boc) which is removed under suitable conditions, for example, with strong acids such as trifluoroacetic acid to form a compound of Formula (I) wherein R^{9a} is H. In an embodiment, Hal² is Br. In an embodiment, Rⁱ and R^{j} are both H. In an embodiment Rⁱ and R^{j} together, form a cycloalkyl ring. The variables, Q, X¹, X², X³, Cy¹ and Cy² are as defined in Formula (I). In an embodiment, the compound of Formula I is the S-enantiomer, R^{7a} is the S-enantionmer,

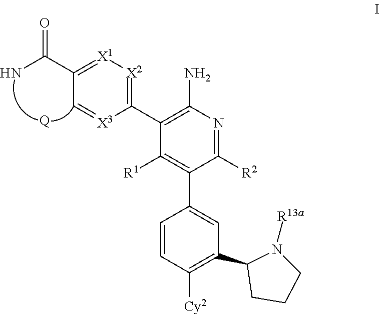

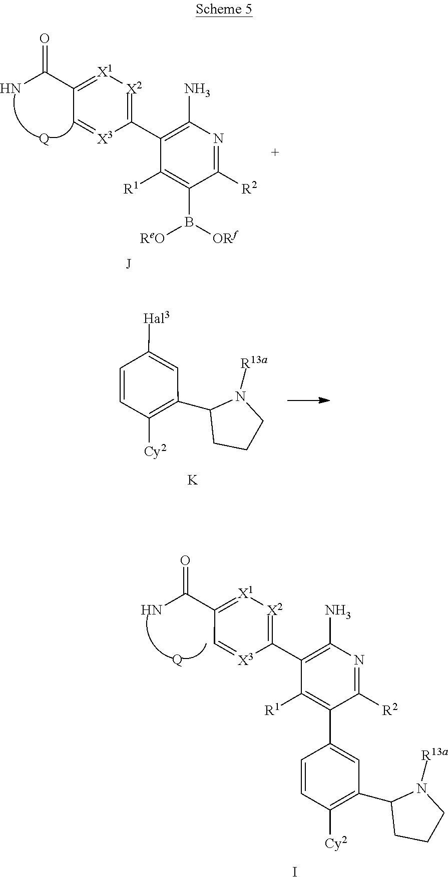

In an embodiment, as shown in Scheme 6, the intermediate compound of Formula (K) in Scheme 5 is synthesized by coupling dihalogenated ester compound of Formula (L) with halogened compound of Formula (M) under suitable coupling conditions such as in the presence of zinc to form halogenated ester compound of Formula (N). The halogenated ester compound of Formula (N) is then reduced under suitable reducing conditions such as in the presence of lithium aluminum hydride to form hydroxy compound of Formula (O) which is subsequently oxidized under suitable oxidizing conditions such as in the presence of manganese dioxide (MnO₂) to provide the halogenated aldehyde compound of Formula (P). The compound of Formula (P) is subsequently coupled with tert-butanesulfinamide (compound of Formula (Q)) to form the aldimine compound of Formula (R) which is further coupled with a (1,3-dioxan-2-ylethyl) (1,3-dioxan-2-ylethyl)magnesium bromide (compound of Formula (S)) under suitable Grignard reaction conditions to provide intermediate compound of Formula (T) which is cyclized under suitable cyclization conditions such as in the presence of trifluoric acid (TFA) and triethylsilane (EtSiH) to form the compound of Formula (K). In an embodiment, the tert-butanesulfinamide (compound of Formula Q) is S-tert-butanesulfinamide and the subsequent compound of Formula (R), (T), and (K) are S-enantiomers. In an embodiment Hal³, Hal⁴ and Hal⁵ are each halogens selected to work in the specific coupling reaction as would be known to those skilled in the art. In an embodiment Hal³ and Hal⁴ are Br and I respectively. In an embodiment, Hal⁵ is any suitable halogen. In an embodiment, Hal⁵ is I. The variables Cy¹ and Cy² are as defined in Formula I.

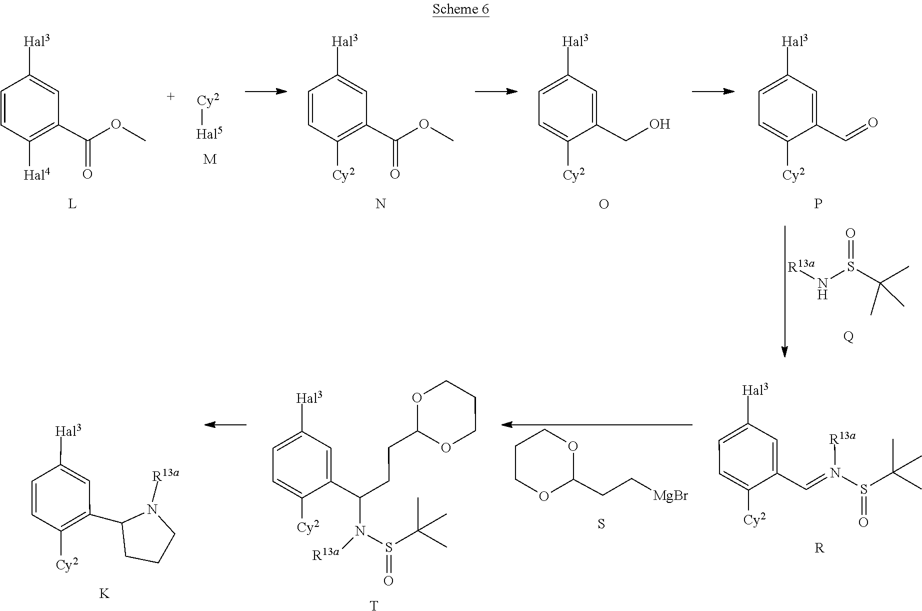

The schemes above are provided for illustration purposes. It will be understood by a person skilled in the art that the use of the most appropriate reagents may vary depend upon of the intermediates compounds of Formula (A)-(T), and that the most appropriate route may also be dependent upon the intermediates and the target compound of Formula (I).

Intermediates of compounds of Formula (A)-(T) are either commercially available or may be prepared using methods known in the art.

Generally the reactions described above are performed in a suitable inert organic solvent and at temperatures and for times that will optimize the yield of the desired compounds. Examples of suitable inert organic solvents include, but are not limited to, dimethylformamide (DMF), dioxane, methylene chloride, chloroform, tetrahydrofuran (THF), toluene, and the like.

Salts of the compounds of the application are generally formed by dissolving the neutral compound in an inert organic solvent and adding either the desired acid or base and isolating the resulting salt by either filtration or other known means.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”.

Prodrugs of the compounds of the present application may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine). Some common esters which have been utilized as prodrugs are phenyl esters, aliphatic (C1-C24) esters, acyloxymethyl esters, carbamates and amino acid esters.

Compounds of the application, or pharmaceutically acceptable salts, solvates and/or prodrug thereof, which comprise deuterium can be prepared according to the processes illustrated in the schemes above, with deuterium being incorporated through commercially available deuterated alkylating agents, by reductions using deuterium analogs of common reducing agents and/or using iminium chemistry through known methods, for example, using the synthetic procedures found in Zhan et al., J. Label Compd. Radiopharm 2014, 57 533-539. Further isotopically-enriched compounds of the application or pharmaceutically acceptable salts, solvates and/or prodrug thereof, can be prepared using conventional techniques well known to those skilled in the art or by processes analogous to those described in the Schemes and Examples herein using suitable isotopically-enriched reagents and/or intermediates.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

The following non-limiting examples are illustrative of the present application.

### EXAMPLES

### A. Preparation of Exemplary Compounds of the Application

### Example 1: 5-(2-amino-5-(1-(1-methylpiperidin-4-yl)-1H-pyrazol-4-yl)pyridin-3-yl)isoindolin-1-one (I-1)

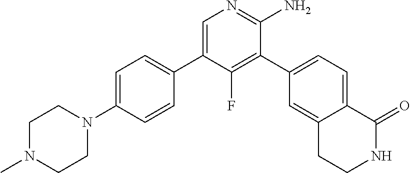 [SMILES: CN1CCN(c2ccc(-c3cnc(N)c(-c4ccc5c(c4)CCNC5=O)c3F)cc2)CC1]

Step 1: 3-Chloro-4-fluoro-5-(4-(4-methylpiperazin-1-yl)phenyl)pyridin-2-amine

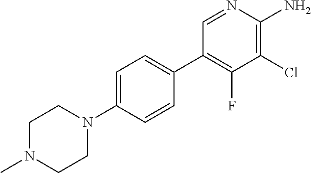 [SMILES: CN1CCN(c2ccc(-c3cnc(N)c(Cl)c3F)cc2)CC1]

In a microwave vial with magnetic stir bar was placed 5-bromo-3-chloro-4-fluoropyridin-2-amine 1 (208 mg, 0.92 mmol), 4-(4-methylpiperazin-1-yl)phenylboronic acid, pinacol ester (307 mg, 1.02 mmol), [1,1′-bis(diphenylphosphino)ferrocene]dichloropalladium(II) (Pd(dppf)Cl₂), (67.5 mg, 0.092 mmol), Cs₂CO₃ (902 mg, 2.77 mmol), 1,2-dimethoxyethane (DME) (8 mL) and water (4 mL). The flask was sealed, with a cap, and then heated to 90° C. in the microwave (high absorbance) for 1 h. LCMS analysis indicated complete conversion and the reaction was concentrated directly onto Celite® using a Biotage® V-10 evaporator and then purified by silica gel chromatography (eluting with CH₂Cl₂/MeOH), followed by reverse phase chromatography (C18, H₂O-MeCN) to provide the title compound (288 mg, 0.90 mmol) in 97% yield as a tan solid. LCMS: [M+H]⁺=321.25.

Step 2 (General Method A): 6-(2-amino-4-fluoro-5-(4-(4-methylpiperazin-1-yl)phenyl)pyridin-3-yl)-3,4-dihydroisoquinolin-1(2H)-one

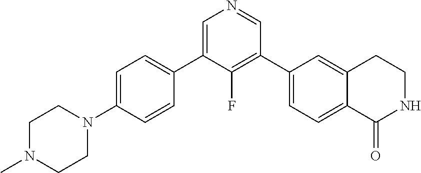 [SMILES: CN1CCN(c2ccc(-c3cncc(-c4ccc5c(c4)CCNC5=O)c3F)cc2)CC1]

In a microwave vial with magnetic stir bar was placed XPhos Pd G2 (2.94 mg, 3.74 μmol), 3-chloro-4-fluoro-5-(4-(4-methylpiperazin-1-yl)phenyl)pyridin-2-amine (60 mg, 0.187 mmol), 3,4-dihydro-1(2H)-isoquinolinone-6-boronic acid pinacol ester (77 mg, 0.281 mmol), butan-1-ol (1.200 mL) and potassium phosphate tribasic reagent grade, >=98% (0.288 mL, 0.374 mmol) (4:1 BuOH/H₂O). The flask was sealed and flushed with nitrogen, then heated to 90° C. for 1 h in the microwave when LCMS indicated complete conversion. The reaction was then concentrated and the crude material was loaded onto Celite® and purified by silica gel chromatography (eluting with DCM-MeOH) to give the crude product. This material was then purified through a PoraPak® RxnCX 6 cc column (acidic PE resin), eluting with 3% NH₄OH in MeOH to give the title compound (15 mg, 19%) as a white solid. ¹H NMR (500 MHz, DMSO-d₆) δ 8.06 (d, J=10.9 Hz, 1H), 7.97 (br s, 1H), 7.94 (d, J=7.8 Hz, 1H), 7.38-7.31 (m, 4H), 6.99 (d, J=8.9 Hz, 2H), 3.41 (td, J=6.5, 2.8 Hz, 2H), 3.18-3.14 (m, 4H), 2.95 (t, J=6.6 Hz, 2H), 2.46-2.43 (m, 4H), 2.22 (s, 3H); LCMS: [M+H]⁺=432.39.

### Example 2: 6-(2-amino-4-fluoro-5-(4-morpholinophenyl)pyridin-3-yl)-3,4-dihydroisoquinolin-1(2H)-one (I-2)

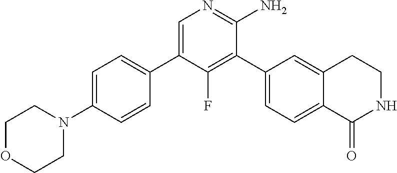 [SMILES: Nc1ncc(-c2ccc(N3CCOCC3)cc2)c(F)c1-c1ccc2c(c1)CCNC2=O]

Step 1: 3-Chloro-4-fluoro-5-(4-morpholinophenyl)pyridin-2-amine

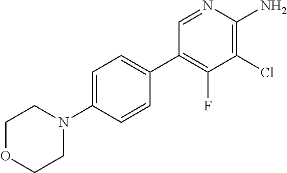 [SMILES: Nc1ncc(-c2ccc(N3CCOCC3)cc2)c(F)c1Cl]

The procedure followed was similar to that of Example 1, Step 1 using 5-bromo-3-chloro-4-fluoropyridin-2-amine (212 mg, 0.94 mmol) and 4-morpholinophenylboronic acid, pinacol ester (299 mg, 1.03 mmol) to provide the title compound (194 mg, 67%) as a grey solid.

Step 2: 6-(2-amino-4-fluoro-5-(4-morpholinophenyl)pyridin-3-yl)-3,4-dihydroisoquinolin-1(2H)-one

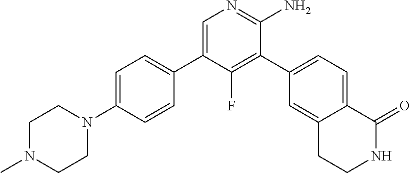 [SMILES: CN1CCN(c2ccc(-c3cnc(N)c(-c4ccc5c(c4)CCNC5=O)c3F)cc2)CC1]

The procedure followed was similar to Example 1, step 2 using XPhos Pd G2 (2.94 mg, 3.74 μmol), 3-chloro-4-fluoro-5-(4-(4-methylpiperazin-1-yl)phenyl)pyridin-2-amine (60 mg, 0.187 mmol), 3,4-dihydro-1(2H)-isoquinolinone-6-boronic acid pinacol ester (77 mg, 0.281 mmol), and aqueous K₃PO₄ (0.288 mL of a 1.3 M solution, 0.374 mmol) and 4:1 BuOH/H₂O (1.5 mL). Purification using silica gel chromatography (eluting with DCM-MeOH) gave material that was then purified through a PoraPak® RxnCX 6 cc column (acidic polyethylene (PE) resin), eluting with 3% NH₄OH in MeOH to afford the title compound (18 mg, 19%) as a white solid. ¹H NMR (500 MHz, DMSO-d₆) δ 8.06 (d, J=10.9 Hz, 1H), 7.97 (br s, 1H), 7.94 (d, J=7.8 Hz, 1H), 7.38-7.31 (m, 4H), 6.99 (d, J=8.9 Hz, 2H), 3.41 (td, J =6.5, 2.8 Hz, 2H), 3.18-3.14 (m, 4H), 2.95 (t, J=6.6 Hz, 2H), 2.46-2.43 (m, 4H), 2.22 (s, 3H); LCMS: [M+H]⁺=432.39.

### Example 3: 6-(2-amino-4-fluoro-5-(4-(isopropylsulfonyl)phenyl)pyridin-3-yl)-3,4-dihydroisoquinolin-1(2H)-one (I-3)

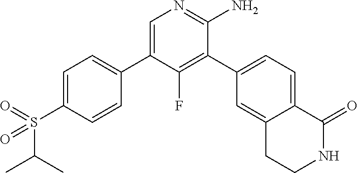 [SMILES: CC(C)S(=O)(=O)c1ccc(-c2cnc(N)c(-c3ccc4c(c3)CCNC4=O)c2F)cc1]

Step 1: 3-chloro-4-fluoro-5-(4-(isopropylsulfonyl)phenyl)pyridin-2-amine

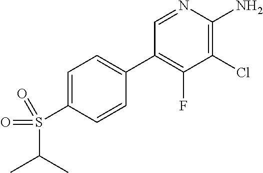 [SMILES: CC(C)S(=O)(=O)c1ccc(-c2cnc(N)c(Cl)c2F)cc1]

A procedure similar to Example 1, Step 1 was employed using 5-bromo-3-chloro-4-fluoropyridin-2-amine (210 mg, 0.93 mmol) and 4-isopropylsulfonylphenylboronic acid, pinacol ester (318 mg, 1.03 mmol) to provide the title compound (247 mg, 81%) as a grey solid. ¹H NMR (500 MHz, DMSO-d₆) δ 8.19 (d, J=10.0 Hz, 1H), 7.90 (d, J=8.4 Hz, 2H), 7.80 (d, J=7.5 Hz, 2H), 6.97 (br s, 2H), 3.46 (dt, J=13.6, 6.8 Hz, 1H), 1.18 (d, J=6.8 Hz, 6H); LCMS: [M+H]⁺=329.15.

Step 2: 6-(2-amino-4-fluoro-5-(4-(isopropylsulfonyl)phenyl)pyridin-3-yl)-3,4-dihydroisoquinolin-1(2H)-one

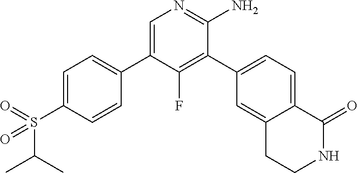 [SMILES: CC(C)S(=O)(=O)c1ccc(-c2cnc(N)c(-c3ccc4c(c3)CCNC4=O)c2F)cc1]

In a microwave vial with a magnetic stir bar was placed 3-chloro-4-fluoro-5-(4-(isopropylsulfonyl)phenyl)pyridin-2-amine (91 mg, 0.277 mmol), 3,4-dihydro-1(2H)-isoquinolinone-6-boronic acid pinacol ester (113 mg, 0.415 mmol), XPhos Pd G2 (10.89 mg, 0.014 mmol), Cs₂CO₃ (271 mg, 0.830 mmol), 1,2-dimethoxyethane (DME) (4 mL) and water (2 mL). The flask was sealed, then heated to 90° C. for 1 h in a microwave apparatus (high absorbance). The reaction then was loaded onto Celite® and purified by reverse phase chromatography (C18, MeCN—H₂O) to give the final product (57 mg, 45%) as a white solid. 1H NMR (500 MHz, DMSO-d₆) δ 8.25 (d, J=10.8 Hz, 1H), 7.99 (br s, 1H), 7.95 (d, J=7.7 Hz, 1H), 7.91-7.87 (m, 2H), 7.81 (d, J=7.9 Hz, 2H), 7.41-7.37 (m, 2H), 6.22 (br s, 2H), 3.48-3.44 (m, 1H), 3.44-3.40 (m, 2H), 2.96 (br t, J=6.5 Hz, 2H), 1.18 (d, J=6.7 Hz, 6H); LCMS: [M+H]⁺=440.18.

### Example 4: (R)-6-(2-amino-4-fluoro-5-(4-(2-isopropylmorpholino)phenyl)pyridin-3-yl)-3,4-dihydroisoquinolin-1(2H)-one (I-4)

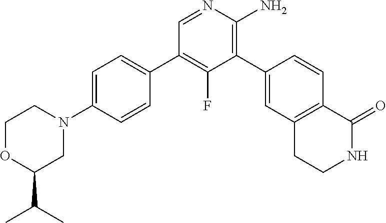 [SMILES: CC(C)[C@@H]1CN(c2ccc(-c3cnc(N)c(-c4ccc5c(c4)CCNC5=O)c3F)cc2)CCO1]

Step 1: (R)-4-(4-chlorophenyl)-2-isopropylmorpholine

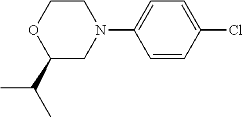 [SMILES: CC(C)[C@@H]1CN(c2ccc(Cl)cc2)CCO1]

A 30 mL vial was charged with 1-chloro-4-iodobenzene (0.50 g, 2.1 mmol), (R)-2-isopropylmorpholine (0.30 g, 2.3 mmol), tris(dibenzylideneacetone)dipalladium (0) (0.06 g, 0.06 mmol), 4,5-bis(diphenylphosphino)-9,9-dimethylxanthene (0.11 g, 0.19 mmol) and CS₂CO₃ (2.05 g, 6.3 mmol). The vial was sealed with a cap and septum and then the reaction vial was evacuated and backfilled with nitrogen. Toluene (7 mL) was added and the reaction vial was evacuated and backfilled with nitrogen an additional time. The reaction was heated at 100° C. for 18 h, cooled to room temperature (RT) and partitioned between EtOAc and water. The layers were separated and the aqueous layer was extracted with additional EtOAc (x2). The combined organic extracts were dried and concentrated onto Celite®. Silica gel chromatography (1-15% EtOAc/hexanes) afforded the product (0.40 g, 79%). LCMS: [M +H]⁺=240.1.

Step 2: (R)-2-isopropyl-4-(4-(4,4,5,5-tetramethyl-1,3,2-dioxaborolan-2-yl)phenyl)morpholine

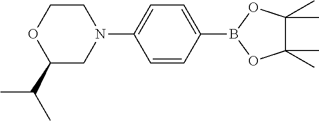 [SMILES: CC(C)[C@@H]1CN(c2ccc(B3OC(C)(C)C(C)(C)O3)cc2)CCO1]

A 30 mL vial was charged with (R)-4-(4-chlorophenyl)-2-isopropylmorpholine (0.40 g, 1.7 mmol), bis(pinacolato)diboron (0.53 g, 2.1 mmol), K₂CO₃ (0.33 g, 3.3 mmol) and XPhos Pd G2 (0.10 g, 0.12 mmol). The vial was evacuated and backfilled with nitrogen. 1,4-Dioxane (5 mL) was added and the vial was evacuated and backfilled an additional time. The reaction was heated at 90° C. for 18 h. After cooling to RT the reaction mixture was concentrated directly onto Celite® and purified by silica gel chromatography (1-15% EtOAc/hexanes) to afford the product (0.45 g, 82%). LCMS: [M+H]⁺=332.1.

Step 3: (R)-3-chloro-4-fluoro-5-(4-(2-isopropylmorpholino)phenyl)pyridin-2-amine

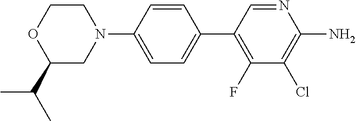 [SMILES: CC(C)[C@@H]1CN(c2ccc(-c3cnc(N)c(Cl)c3F)cc2)CCO1]

A 30 mL vial was charged with 5-bromo-3-chloro-4-fluoropyridin-2-amine (0.030 g, 0.13 mmol), (R)-2-isopropyl-4-(4-(4,4,5,5-tetramethyl-1,3,2-dioxaborolan-2-yl)phenyl)morpholine (0.050 g, 0.15 mmol), [1,1′-bis(diphenylphosphino)ferrocene]dichloropalladium(II) (10 mg, 0.013 mmol) and cesium carbonate (0.13 g, 0.40 mmol). The vial was sealed with a cap and septum and the reaction vial was evacuated and backfilled with nitrogen. 1,4-Dioxane (1 mL) and H₂O (0.5 mL) were added and the reaction vial was evacuated and backfilled with nitrogen an additional time. The reaction was heated at 90° C. for 18 h. After cooling to RT the reaction mixture was concentrated directly onto Celite® and purified by silica gel chromatography (eluting with 0.5%-6% MeOH/DCM+0.5% NH₄OH) to afford the product (0.046 g, quantitative yield). LCMS: [M+H]⁺=350.5.

Step 4: (R)-6-(2-amino-4-fluoro-5-(4-(2-isopropylmorpholino)phenyl)pyridin-3-yl)-3,4-dihydroisoquinolin-1(2H)-one

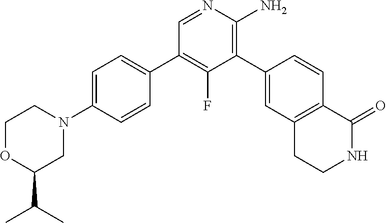 [SMILES: CC(C)[C@@H]1CN(c2ccc(-c3cnc(N)c(-c4ccc5c(c4)CCNC5=O)c3F)cc2)CCO1]

A 30 mL vial was charged with (R)-3-chloro-4-fluoro-5-(4-(2-isopropylmorpholino)phenyl)pyridin-2-amine (0.050 g, 0.14 mmol), 3,4-dihydro-1(2H)-isoquinolinone-6-boronic acid pinacol ester (0.046 g, 0.17 mmol) and XPhos Pd G2 (0.011 g, 0.014 mmol). The vial was sealed with a cap and septum and the reaction vial was evacuated and backfilled with nitrogen. 1,4-Dioxane (1 mL) and aqueous K₃PO₄ (0.27 mL of a 1.3 M solution, 0.35 mmol) were added and the reaction vial was evacuated and backfilled with nitrogen an additional time. The reaction was heated conventionally at 90° C. for 18 h. After cooling to RT, the reaction mixture was concentrated directly onto Celite® and purified by silica gel chromatography (eluting with 0.5-7.5% MeOH/DCM+0.5% NH₄OH) followed by reverse phase chromatography (C18; 5-60% MeCN/water+0.1% formic acid) to afford the title compound (0.023 g, 36%). ¹H NMR (500 MHz, DMSO-d₆) δ 8.07 (d, J=10.8 Hz, 1H), 7.9-8.0 (m, 2H), 7.3-7.4 (m, 4H), 7.02 (d, J=8.8 Hz, 2H), 5.85 (s, 2H), 3.9-4.0 (m, 1H), 3.5-3.6 (m, 4H), 3.42 (dt, J=6.5, 2.7 Hz, 3H), 3.24 (ddd, 2H, J=10.3, 6.3, 2.2 Hz), 2.95 (br t, J =6.5 Hz, 2H), 2.67 (dt, J=11.8, 3.4 Hz, 1H), 2.4-2.5 (m, 2H), 1.7-1.8 (m, 1H), 0.96 (dd, J=6.7, 4.4 Hz, 6H); LCMS: [M+H]⁺=461.7.

### Example 5: (S)-6-(2-amino-4-fluoro-5-(4-(2-isopropylmorpholino)phenyl)pyridin-3-yl)-3,4-dihydroisoquinolin-1(2H)-one (I-5)

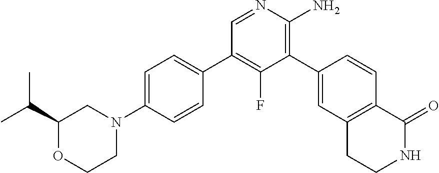 [SMILES: CC(C)[C@H]1CN(c2ccc(-c3cnc(N)c(-c4ccc5c(c4)CCNC5=O)c3F)cc2)CCO1]

Step 1: (S)-4-(4-chlorophenyl)-2-isopropylmorpholine

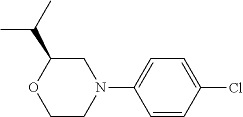 [SMILES: CC(C)[C@H]1CN(c2ccc(Cl)cc2)CCO1]

A 30 mL vial was charged with 1-chloro-4-iodobenzene (0.50 g, 2.1 mmol), (S)-2-isopropylmorpholine (0.30 g, 2.3 mmol), tris(dibenzylideneacetone)dipalladium (0) (0.06 g, 0.06 mmol), 4,5-bis(diphenylphosphino)-9,9-dimethylxanthene (0.11 g, 0.19 mmol) and Cs₂CO₃ (2.05 g, 6.3 mmol). The vial was sealed with a cap and septum and then the reaction vial was evacuated and backfilled with nitrogen. Toluene (7 mL) was added and the reaction vial was evacuated and backfilled with nitrogen an additional time. The reaction was heated at 100° C. for 18 h. The reaction mixture was cooled to RT and partitioned between EtOAc and water. The layers were separated and the aqueous layer was extracted with additional EtOAc (x2). The combined organic extracts were dried and concentrated onto Celite®. Silica gel chromatography (eluting with 1-15% EtOAc/hexanes) afforded the product (0.40 g, 80%). LCMS: [M+H]⁺=240.0.

Step 2: (S)-2-isopropyl-4-(4-(4,4,5,5-tetramethyl-1,3,2-dioxaborolan-2-yl)phenyl)morpholine

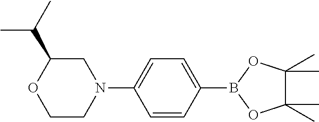 [SMILES: CC(C)[C@H]1CN(c2ccc(B3OC(C)(C)C(C)(C)O3)cc2)CCO1]

A 30 mL vial was charged with (S)-4-(4-chlorophenyl)-2-isopropylmorpholine (0.40 g, 1.7 mmol), bis(pinacolato)diboron (0.53 g, 2.1 mmol), KOAc (0.33 g, 3.3 mmol) and XPhos Pd G2 (0.10 g, 0.12 mmol). The vial was evacuated and backfilled with nitrogen. 1,4-Dioxane (5 mL) was added and the vial was evacuated and backfilled an additional time. The reaction was heated at 90° C. for 18 h. After cooling to RT the reaction mixture was concentrated directly onto Celite® and purified by silica gel chromatography (eluting with 1-15% EtOAc/hexanes) to afford the product (0.47 g, 85%). LCMS: [M+H]⁺=332.1.

Step 3: (S)-3-chloro-4-fluoro-5-(4-(2-isopropylmorpholino)phenyl)pyridin-2-amine

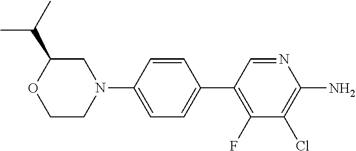 [SMILES: CC(C)[C@H]1CN(c2ccc(-c3cnc(N)c(Cl)c3F)cc2)CCO1]

A 30 mL vial was charged with 5-bromo-3-chloro-4-fluoropyridin-2-amine (0.030 g, 0.13 mmol), (S)-2-isopropyl-4-(4-(4,4,5,5-tetramethyl-1,3,2-dioxaborolan-2-yl)phenyl)morpholine (0.050 g, 0.15 mmol), [1,1-bis(diphenylphosphino)ferrocene]dichloropalladium(II) (10 mg, 0.013 mmol) and CS₂CO₃ (0.13 g, 0.40 mmol). The vial was sealed with a cap and septum and the reaction vial was evacuated and backfilled with nitrogen. 1,4-Dioxane (1 mL) and H₂O (0.5 mL) were added and the reaction vial was evacuated and backfilled with nitrogen an additional time. The reaction was heated at 90° C. for 18 h. After cooling to RT the reaction mixture was concentrated directly onto Celite® and purified by silica gel chromatography (eluting with 0.5%-6% MeOH/DCM+0.5% NH₄OH) to afford the product (0.052 g, quantitative yield). LCMS: [M+H]⁺=350.4.

Step 4: (S)-6-(2-amino-4-fluoro-5-(4-(2-isopropylmorpholino)phenyl)pyridin-3-yl)-3,4-dihydroisoquinolin-1(2H)-one

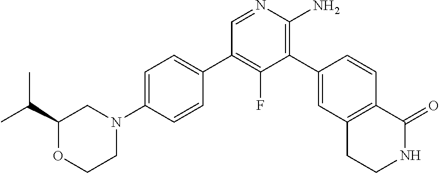 [SMILES: CC(C)[C@H]1CN(c2ccc(-c3cnc(N)c(-c4ccc5c(c4)CCNC5=O)c3F)cc2)CCO1]

A 30 mL vial was charged with (S)-3-chloro-4-fluoro-5-(4-(2-isopropylmorpholino)phenyl)pyridin-2-amine (0.052 g, 0.15 mmol), 3,4-dihydro-1(2H)-isoquinolinone-6-boronic acid pinacol ester (0.049 g, 0.18 mmol) and XPhos Pd G2 (0.012 g, 0.015 mmol). The vial was sealed with a cap and septum and the reaction vial was evacuated and backfilled with nitrogen. 1,4-Dioxane (1 mL) and aqueous K₃PO₄ (0.29 mL of a 1.3 M solution, 0.35 mmol) were added and the reaction vial was evacuated and backfilled with nitrogen an additional time. The reaction was heated at 90° C. for 18 h. After cooling to RT the reaction mixture was concentrated directly onto Celite® and purified by silica gel chromatography (eluting with 0.5-7.5% MeOH/DCM+0.5% NH₄OH) followed by reverse phase chromatography (C18; 5-60% MeCN/water +0.1% formic acid) to afford the title compound (29 mg, 42%). ¹H NMR (500 MHz, DMSO-d₆) δ 8.07 (d, J=10.8 Hz, 1H), 7.9-8.0 (m, 2H), 7.3-7.4 (m, 4H), 7.02 (d, J=8.8 Hz, 2H), 5.85 (s, 2H), 3.9-4.0 (m, 1H), 3.5-3.6 (m, 4H), 3.42 (dt, J=6.5, 2.7 Hz, 3H), 3.24 (ddd, J=10.3, 6.3, 2.2 Hz, 2H), 2.95 (br t, J=6.5 Hz, 2H), 2.67 (dt, J=11.8, 3.4 Hz, 1H), 2.4-2.5 (m, 2H), 1.7-1.8 (m, 1H), 0.96 (dd, J=6.7, 4.4 Hz, 6H); LCMS: [M+H]⁺=461.7.

### Example 6: (R)-6-(2-amino-4-fluoro-5-(2-fluoro-4-(2-isopropylmorpholino)phenyl)pyridin-3-yl)-3,4-dihydroisoquinolin-1(2H)-one (I-6)

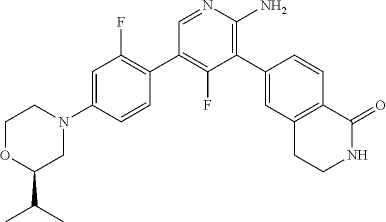 [SMILES: CC(C)[C@@H]1CN(c2ccc(-c3cnc(N)c(-c4ccc5c(c4)CCNC5=O)c3F)c(F)c2)CCO1]

Step 1: (R)-4-(4-chloro-3-fluorophenyl)-2-isopropylmorpholine

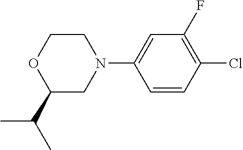 [SMILES: CC(C)[C@@H]1CN(c2ccc(Cl)c(F)c2)CCO1]

A30 mL vial was charged with 4-bromo-1-chloro-2-fluorobenzene (0.50 g, 2.4 mmol), (R)-2-isopropylmorpholine (0.34 g, 2.6 mmol), tris(dibenzylideneacetone)dipalladium (0) (0.066 g, 0.072 mmol), 4,5-bis(diphenylphosphino)-9,9-dimethylxanthene (0.12 g, 0.22 mmol) and Cs₂CO₃ (2.3 g, 7.2 mmol). The vial was sealed with a cap and septum and then the reaction vial was evacuated and backfilled with nitrogen. Toluene (6 mL) was added and the reaction vial was evacuated and backfilled with nitrogen an additional time. The reaction was heated conventionally at 100° C. for 18 h. The reaction mixture was cooled to RT and partitioned between EtOAc and water. The layers were separated and the aqueous layer was extracted with additional EtOAc (x2). The combined organic extracts were dried and concentrated onto Celite®. Silica gel chromatography (5% EtOAc/hexanes) afforded the product (0.67 g, quantitative yield). LCMS: [M+H]⁺=258.1.

Step 2: (R)-4-(3-fluoro-4-(4,4,5,5-tetramethyl-1,3,2-dioxaborolan-2-yl)phenyl)-2-isopropylmorpholine

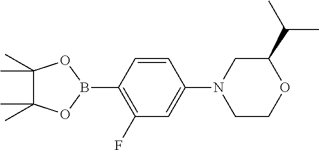 [SMILES: CC(C)[C@@H]1CN(c2ccc(B3OC(C)(C)C(C)(C)O3)c(F)c2)CCO1]

A 30 mL vial was charged with (R)-4-(4-chloro-3-fluorophenyl)-2-isopropylmorpholine (0.67 g, 2.6 mmol), bis(pinacolato)diboron (0.83 g, 3.3 mmol), KOAc (0.51 g, 5.2 mmol) and XPhos Pd G2 (0.15 g, 0.20 mmol). The vial was evacuated and backfilled with nitrogen. 1,4-Dioxane (8 mL) was added and the vial was evacuated and backfilled an additional time. The reaction was heated at 90° C. for 18 h. After cooling to RT the reaction mixture was concentrated directly onto Celite® and purified by silica gel chromatography (eluting with 1-15% EtOAc/hexanes) to afford the product (0.91 g, quantitative yield). LCMS: [M+H]⁺=350.2.

Step 3: (R)-3-chloro-4-fluoro-5-(2-fluoro-4-(2-isopropylmorpholino)phenyl)pyridin-2-amine

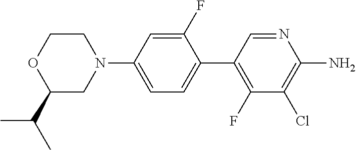 [SMILES: CC(C)[C@@H]1CN(c2ccc(-c3cnc(N)c(Cl)c3F)c(F)c2)CCO1]

A 30 mL vial was charged with 5-bromo-3-chloro-4-fluoropyridin-2-amine (0.030 g, 0.13 mmol), (R)-4-(3-fluoro-4-(4,4,5,5-tetramethyl-1,3,2-dioxaborolan-2-yl)phenyl)-2-isopropylmorpholine (0.051 g, 0.15 mmol), [1,1′-bis(diphenylphosphino)ferrocene]dichloropalladium(II) (10 mg, 0.013 mmol) and cesium carbonate (0.13 g, 0.40 mmol). The vial was sealed with a cap and septum and the reaction vial was evacuated and backfilled with nitrogen. 1,4-Dioxane (1 mL) and H₂O (0.5 mL) were added and the reaction vial was evacuated and backfilled with nitrogen an additional time. The reaction was heated at 90° C. for 18 h. After cooling to RT the reaction mixture was concentrated directly onto elite and purified by silica gel chromatography (eluting with 0.5%-6% MeOH/DCM+0.5% NH₄OH) to afford the product (0.030 g, 61%). LCMS: [M+H]⁺=368.6.

Step 4: (R)-6-(2-amino-4-fluoro-5-(2-fluoro-4-(2-isopropylmorpholino)phenyl)pyridin-3-yl)-3,4-dihydroisoquinolin-1(2H)-one

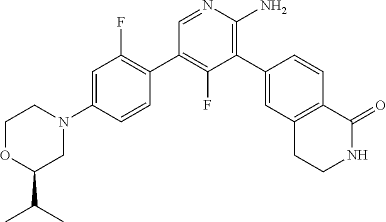 [SMILES: CC(C)[C@@H]1CN(c2ccc(-c3cnc(N)c(-c4ccc5c(c4)CCNC5=O)c3F)c(F)c2)CCO1]

A 30 mL vial was charged with (R)-3-chloro-4-fluoro-5-(2-fluoro-4-(2-isopropylmorpholino)phenyl)pyridin-2-amine (0.030 g, 0.082 mmol), 3,4-dihydro-1(2H)-isoquinolinone-6-boronic acid pinacol ester (0.027 g, 0.098 mmol) and XPhos Pd G2 (7 mg, 8.1 μmol). The vial was sealed with a cap and septum and the reaction vial was evacuated and backfilled with nitrogen. 1,4-Dioxane (1 mL) and 1.3 M aqueous K₃PO₄ (0.16 mL of a 1.3 M solution, 0.21 mmol) were added and the reaction vial was evacuated and backfilled with N₂ an additional time. The reaction was heated at 90° C. for 18 h. After cooling to RT the reaction mixture was concentrated directly onto Celite® and purified by silica gel chromatography (eluting with 0.5-7.5% MeOH/DCM+0.5% NH₄OH) followed by reverse phase chromatography (C18; 5-60% MeCN/water+0.1% formic acid) to afford the title compound (14 mg, 36%). ¹H NMR (500 MHz, DMSO-d₆) δ 7.9-8.0 (m, 3H), 7.34 (br s, 2H), 7.2-7.3 (m, 1H), 6.8-6.9 (m, 2H), 5.94 (br s, 2H), 3.9-4.0 (m, 2H), 3.6-3.6 (m, 6H), 3.2-3.3 (m, 2H), 2.95 (br d, J=5.5 Hz, 3H), 2.7-2.7 (m, 2H), 1.7-1.8 (m, 1H), 0.9-1.0 (m, 6H); LCMS [M +H]⁺=479.5.

### Example 7: (S)-6-(2-amino-4-fluoro-5-(2-fluoro-4-(2-isopropylmorpholino)phenyl)pyridin-3-yl)-3,4-dihydroisoquinolin-1(2H)-one (I-7)

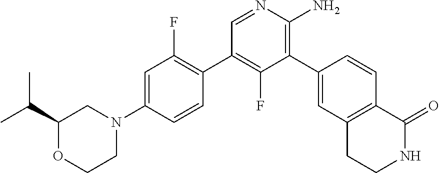 [SMILES: CC(C)[C@H]1CN(c2ccc(-c3cnc(N)c(-c4ccc5c(c4)CCNC5=O)c3F)c(F)c2)CCO1]

Step 1: (S)-4-(4-chloro-3-fluorophenyl)-2-isopropylmorpholine

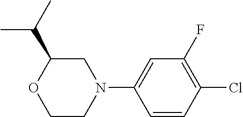 [SMILES: CC(C)[C@H]1CN(c2ccc(Cl)c(F)c2)CCO1]

A 30 mL vial was charged with 4-bromo-1-chloro-2-fluorobenzene (0.50 g, 2.4 mmol), (S)-2-isopropylmorpholine (0.34 g, 2.6 mmol), tris(dibenzylideneacetone)dipalladium (0) (0.066 g, 0.072 mmol), 4,5-bis(diphenylphosphino)-9,9-dimethylxanthene (0.12 g, 0.22 mmol) and Cs₂CO₃ (2.3 g, 7.2 mmol). The vial was sealed with a cap and septum and then the reaction vial was evacuated and backfilled with nitrogen. Toluene (6 mL) was added and the reaction vial was evacuated and backfilled with nitrogen an additional time. The reaction was heated at 100° C. for 18 h, cooled to RT and partitioned between EtOAc and water. The layers were separated and the aqueous layer was extracted with additional EtOAc (x2). The combined organic extracts were dried and concentrated onto Celite®. Silica gel chromatography (eluting with 5% EtOAc/hexanes) afforded the product (0.78 g, quantitative yield). LCMS: [M+H]⁺=258.2.

Step 2: (S)-4-(3-fluoro-4-(4,4,5,5-tetramethyl-1,3,2-dioxaborolan-2-yl)phenyl)-2-isopropylmorpholine

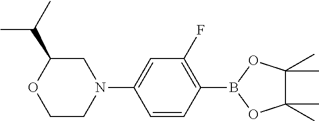 [SMILES: CC(C)[C@H]1CN(c2ccc(B3OC(C)(C)C(C)(C)O3)c(F)c2)CCO1]

A 30 mL vial was charged with (S)-4-(4-chloro-3-fluorophenyl)-2-isopropylmorpholine (0.78 g, 3.0 mmol), bis(pinacolato)diboron (0.96 g, 3.8 mmol), KOAc (0.60 g, 6.1 mmol) and XPhos Pd G2 (0.18 g, 0.23 mmol). The vial was evacuated and backfilled with nitrogen. 1,4-Dioxane (8 mL) was added and the vial was evacuated and backfilled an additional time. The reaction was heated at 90° C. for 18 h, cooled to RT and concentrated directly onto Celite®. Purification by flash chromatography (1-15% EtOAc/hexanes) afforded the product (0.81 g, 76%). LCMS: [M+H]⁺=350.2.

Step 3: (S)-3-chloro-4-fluoro-5-(2-fluoro-4-(2-isopropylmorpholino)phenyl)pyridin-2-amine

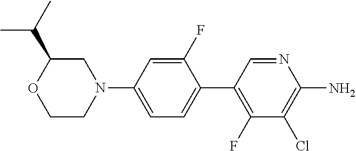 [SMILES: CC(C)[C@H]1CN(c2ccc(-c3cnc(N)c(Cl)c3F)c(F)c2)CCO1]

A 30 mL vial was charged with 5-bromo-3-chloro-4-fluoropyridin-2-amine (0.030 g, 0.13 mmol), (S)-4-(3-fluoro-4-(4,4,5,5-tetramethyl-1,3,2-dioxaborolan-2-yl)phenyl)-2-isopropylmorpholine (0.051 g, 0.15 mmol), [1,1′-bis(diphenylphosphino)ferrocene]dichloropalladium(II) (10 mg, 0.013 mmol) and cesium carbonate (0.13 g, 0.40 mmol). The vial was sealed with a cap and septum and the reaction vial was evacuated and backfilled with nitrogen. 1,4-Dioxane (1 mL) and Water (0.5 mL) were added and the reaction vial was evacuated and backfilled with nitrogen an additional time. The reaction was heated conventionally at 90° C. for 18 h. After cooling to RT the reaction mixture was concentrated directly onto Celite® and purified by silica gel chromatography (eluting with 0.5%-6% MeOH/DCM+0.5% NH₄OH) to afford the product (0.031 g, 63%). LCMS: [M+H]⁺=368.5.

Step 4: (S)-6-(2-amino-4-fluoro-5-(2-fluoro-4-(2-isopropylmorpholino)phenyl)pyridin-3-yl)-3,4-dihydroisoquinolin-1(2H)-one

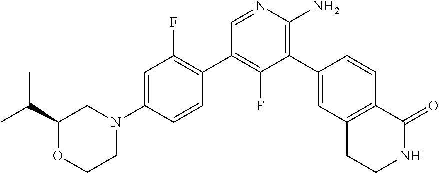 [SMILES: CC(C)[C@H]1CN(c2ccc(-c3cnc(N)c(-c4ccc5c(c4)CCNC5=O)c3F)c(F)c2)CCO1]

A 30 mL vial was charged with (S)-3-chloro-4-fluoro-5-(2-fluoro-4-(2-isopropylmorpholino)phenyl)pyridin-2-amine (0.031 g, 0.084 mmol), 3,4-dihydro-1(2H)-isoquinolinone-6-boronic acid pinacol ester (0.028 g, 0.10 mmol) and XPhos Pd G2 (7 mg, 8.4 μmol). The vial was sealed with a cap and septum and the reaction vial was evacuated and backfilled with nitrogen. 1,4-Dioxane (1 mL) and 1.3 M aqueous K₃PO₄ (0.16 mL, 0.21 mmol) were added and the reaction vial was evacuated and backfilled with nitrogen an additional time. The reaction was heated at 90° C. for 18 h and allowed to cool to RT. The mixture was concentrated directly onto Celite® and purified by silica gel chromatography (eluting with 0.5-7.5% MeOH/DCM+0.5% NH₄OH) followed by reverse phase chromatography (C18; 5-60% MeCN/water+0.1% formic acid) to afford the title compound (11 mg, 27%). ¹H NMR (500 MHz, DMSO-d₆) δ 7.9-8.0 (m, 3H), 7.34 (br s, 2H), 7.2-7.3 (m, 1H), 6.8-6.9 (m, 2H), 5.94 (br s, 2H), 3.9-4.0 (m, 2H), 3.6-3.6 (m, 5H), 3.2-3.2 (m, 3H), 2.95 (br d, 3H, J=5.7 Hz), 2.7-2.7 (m, 2H), 1.7-1.8 (m, 1H), 0.95 (br d, 6H, J=6.5 Hz); LCMS: [M +H]⁺=479.7.

### Example 8: 6-(2-amino-6-fluoro-5-(4-(4-methylpiperazin-1-yl)phenyl)pyridin-3-yl)-3,4-dihydroisoquinolin-1(2H)-one (I-8)

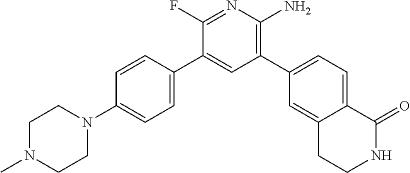 [SMILES: CN1CCN(c2ccc(-c3cc(-c4ccc5c(c4)CCNC5=O)c(N)nc3F)cc2)CC1]

Step 1: 5-bromo-6-fluoropyridin-2-amine

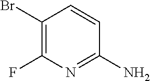

To a solution of 6-fluoropyridin-2-amine (3 g, 26.8 mmol) in acetonitrile (100 mL) was added NBS (4.7 g, 26.8 mmol) in acetonitrile (80 mL) dropwise for 30 min at 0° C. and stirred for another 30 min under argon atmosphere at RT. The solvent was evaporated under reduced pressure to give crude product which was purified by column chromatography (silica gel, 100-200 mesh) using 10% EtOAc in pet ether as an eluent to afford the product (4.4 g, 88%) as a yellow solid. LCMS: [M+H]⁺=191.12.

Step 2: Synthesis of 5-bromo-3-chloro-6-fluoropyridin-2-amine

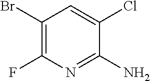

To a solution of 5-bromo-6-fluoropyridin-2-amine (2 g, 10.5 mmol) in acetonitrile (30 mL) was added NCS (1.4 g, 10.5 mmol) portionwise, and the suspension was refluxed for 2 h at 90° C. under argon atmosphere before cooling to RT. The reaction mixture was poured into ice-cold water (100 mL), and the resulting precipitate was collected to give the crude compound. Washing with n-pentane (100 mL) afforded the product (1.1 g, 48%) as an off-white solid. LCMS: [M+H]⁺=225.09

Step 3: 3-chloro-6-fluoro-5-(4-(4-methylpiperazin-1-yl)phenyl)pyridin-2-amine

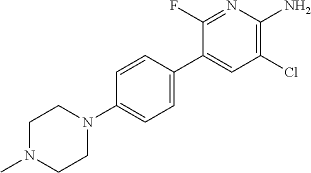 [SMILES: CN1CCN(c2ccc(-c3cc(Cl)c(N)nc3F)cc2)CC1]

In a microwave vial with magnetic stir bar was placed 5-bromo-3-chloro-6-fluoropyridin-2-amine (260 mg, 1.15 mmol), [1,1′-bis(diphenylphosphino)ferrocene]dichloropalladium(II) (84 mg, 0.115 mmol), CSCO₃ (1127 mg, 3.46 mmol), DME (4 mL) and water (2 mL). The flask was sealed, then heated to 90° C. for 1 h in the microwave (high absorbance) when LCMS indicated complete conversion. The reaction was loaded onto Celite® and purified by reverse phase chromatography (C18, MeCN—H₂O) to give the product (0.630 mmol, 55% yield) as a white solid. ¹H NMR (500 MHz, DMSO-d₆) δ 7.80 (d, J=8.9 Hz, 1H), 7.35 (d, J=7.6 Hz, 2H), 6.96 (d, J=8.9 Hz, 2H), 6.66 (s, 2H), 3.18-3.14 (m, 4H), 2.46-2.42 (m, 4H), 2.22 (s, 3H); LCMS: [M+1]⁺=323.28.

Step 4: 6-(2-amino-6-fluoro-5-(4-(4-methylpiperazin-1-yl)phenyl)pyridin-3-yl)-3,4-dihydroisoquinolin-1(2H)-one

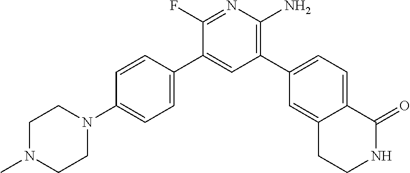 [SMILES: CN1CCN(c2ccc(-c3cc(-c4ccc5c(c4)CCNC5=O)c(N)nc3F)cc2)CC1]

In a microwave vial with magnetic stir bar was placed 3-chloro-6-fluoro-5-(4-(4-methylpiperazin-1-yl)phenyl)pyridin-2-amine (62 mg, 0.194 mmol), 3,4-dihydro-1(2H)-isoquinolinone-6-boronic acid pinacol ester (69.5 mg, 0.255 mmol), XPhos Pd G2 (6.68 mg, 8.49 μmol), CSCO₃ (166 mg, 0.509 mmol), DME (4 mL) and water (2 mL). The flask was sealed, then heated to 90° C. for 1 h in a microwave (high absorbance) until LCMS indicated complete conversion. The reaction was loaded onto Celite® and purified by reverse phase chromatography (C18, MeCN—H₂O) to give the title compound (57 mg, 78%) as a grey solid. ¹H NMR (500 MHz, DMSO-d₆) δ 8.02-7.93 (m, 2H), 7.63 (d, J=10.3 Hz, 1H), 7.54-7.48 (m, 2H), 7.45 (br d, J=7.9 Hz, 2H), 7.02 (br d, J=8.9 Hz, 2H), 6.21 (s, 2H), 3.45 (br dd, J=6.3, 2.3 Hz, 2H), 3.22-3.18 (m, 4H), 3.00 (br t, J=6.4 Hz, 2H), 2.55 (br d, J=1.6 Hz, 2H), 2.27 (s, 3H); LCMS: [M+H]⁺=432.28.

### Example 9: (R)-6-(2-amino-6-fluoro-5-(4-(2-isopropylmorpholino)phenyl)pyridin-3-yl)-3,4-dihydroisoquinolin-1(2H)-one (I-9)

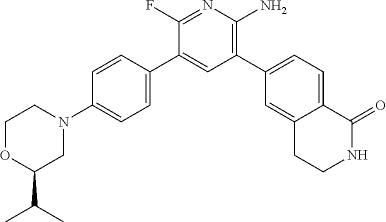 [SMILES: CC(C)[C@@H]1CN(c2ccc(-c3cc(-c4ccc5c(c4)CCNC5=O)c(N)nc3F)cc2)CCO1]

Step 1: (R)-3-chloro-6-fluoro-5-(4-(2-isopropylmorpholino)phenyl)pyridin-2-amine

A 30 mL vial was charged with 5-bromo-3-chloro-6-fluoropyridin-2-amine (0.040 g, 0.18 mmol, prepared according to Example 8), (R)-2-isopropyl-4-(4-(4,4,5,5-tetramethyl-1,3,2-dioxaborolan-2-yl)phenyl)morpholine (0.070 g, 0.20 mmol) and [1,12-bis(diphenylphosphino)ferrocene]dichloropalladium(II), DCM complex (0.014 g, 0.018 mmol). The vial was sealed with a cap and septum and the reaction vial was evacuated and backfilled with nitrogen. 1,4-Dioxane (2 mL) and 2 M aqueous Na₂CO₃ (0.27 mL, 0.53 mmol) were added and the reaction vial was evacuated and backfilled with nitrogen an additional time. The reaction was heated at 85° C. for 18 h. After cooling to RT the reaction mixture was concentrated directly onto Celite® and purified by silica gel chromatography (eluting with 5-95% EtOAc/hexanes) to afford the product (0.030 g, 48%). LCMS: [M+H]⁺=350.4.

Step 2: (R)-6-(2-amino-6-fluoro-5-(4-(2-isopropylmorpholino)phenyl)pyridin-3-yl)-3,4-dihydroisoquinolin-1(2H)-one

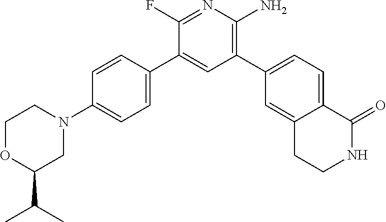 [SMILES: CC(C)[C@@H]1CN(c2ccc(-c3cc(-c4ccc5c(c4)CCNC5=O)c(N)nc3F)cc2)CCO1]

A 30 mL vial was charged with (R)-3-chloro-6-fluoro-5-(4-(2-isopropylmorpholino)phenyl)pyridin-2-amine (0.030 g, 0.086 mmol), 3,4-dihydro-1(2H)-isoquinolinone-6-boronic acid pinacol ester (0.035 g, 0.13 mmol) and XPhos Pd G2 (7 mg, 8.6 μmol). The vial was sealed with a cap and septum and the reaction vial was evacuated and backfilled with nitrogen. 1,4-Dioxane (1 mL) and aqueous K3PO4 (0.17 mL of a 1.3 M solution, 0.21 mmol) were added and the reaction vial was evacuated and backfilled with nitrogen an additional time. The reaction was heated at 90° C. for 18 h. After cooling to RT the reaction mixture was concentrated directly onto Celite® and purified by silica gel chromatography (0.5-9.5% MeOH/DCM+0.5% NH4OH) to afford the product (0.031 g, 78%). 1H NMR (500 MHz, DMSO-d6) δ 7.9-8.0 (m, 2H), 7.58 (br d, 1H, J=9.8 Hz), 7.4-7.5 (m, 4H), 7.01 (br d, 2H, J=8.3 Hz), 6.18 (br s, 2H), 3.97 (br d, 1H, J=10.3 Hz), 3.59 (br d, 3H, J=12.5 Hz), 3.53 (br d, 2H, J=11.7 Hz), 3.40 (br d, 4H, J=4.8 Hz), 3.2-3.3 (m, 2H), 2.95 (br s, 2H), 2.66 (br t, 1H, J=11.1 Hz), 2.44 (br t, 1H, J=11.1 Hz), 1.7-1.8 (m, 1H), 0.96 (br s, 6H); LCMS [M+H]⁺=461.4.

### Example 10: (S)-6-(2-amino-6-fluoro-5-(4-(2-isopropylmorpholino)phenyl)pyridin-3-yl)-3,4-dihydroisoquinolin-1(2H)-one (I-10)

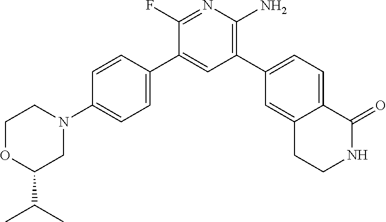 [SMILES: CC(C)[C@H]1CN(c2ccc(-c3cc(-c4ccc5c(c4)CCNC5=O)c(N)nc3F)cc2)CCO1]

Step 1: (S)-2-isopropyl-4-(4-(4,4,5,5-tetramethyl-1,3,2-dioxaborolan-2-yl)phenyl)morpholine

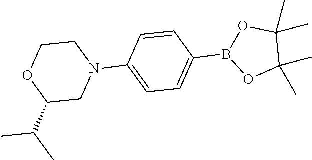 [SMILES: CC(C)[C@H]1CN(c2ccc(B3OC(C)(C)C(C)(C)O3)cc2)CCO1]

A 30 ml vial was charged with (S)-4-(4-chlorophenyl)-2-isopropylmorpholine (0.404 g, 1.685 mmol), Bis(pinacolato)diboron (0.535 g, 2.106 mmol), Potassium acetate (0.331 g, 3.37 mmol) and XPhos Pd G2 (0.099 g, 0.126 mmol). The vial was evacuated and backfilled with nitrogen. 1,4-Dioxane (5 ml) was added and the vial was evacuated and backfilled an additional time. The reaction mixture was then heated to 90° C. in an aluminum block for 18 h. The reaction mixture was concentrated onto celite and purified by flash chromatography (eluting with 5-95% EtOAc/hexanes) to afford the product (0.472 g, 85% yield).

Step 2: (S)-3-chloro-6-fluoro-5-(4-(2-isopropylmorpholino)phenyl)pyridin-2-amine

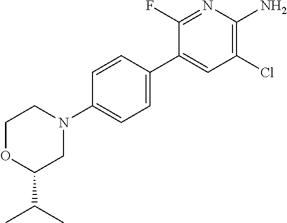 [SMILES: CC(C)[C@H]1CN(c2ccc(-c3cc(Cl)c(N)nc3F)cc2)CCO1]

A 30 mL vial was charged with 5-bromo-3-chloro-6-fluoropyridin-2-amine (0.040 g, 0.177 mmol), (S)-2-isopropyl-4-(4-(4,4,5,5-tetramethyl-1,3,2-dioxaborolan-2-yl)phenyl)morpholine (0.068 g, 0.204 mmol) and [1,12-bis(diphenylphosphino)ferrocene]dichloropalladium(II), DCM complex (0.014 g, 0.018 mmol). The vial was sealed with a septum cap, evacuated and backfilled with nitrogen. 1,4-Dioxane (2 ml) and a 2M aqueous solution of saturated aqueous Na₂CO₃ (0.266 ml, 0.532 mmol) were added and the vial was evacuated and backfilled an additional time. The reaction mixture was heated to 85° C. in an aluminum block for 18 h. LCMS [01] indicated desired product formation. The reaction mixture was concentrated onto celite and purified by flash [5-95% EtOAc/hexanes] to afford the product (28 mg, 45.1% yield). LCMS: [M+H]⁺=350.36.

Step 3: (S)-6-(2-amino-6-fluoro-5-(4-(2-isopropylmorpholino)phenyl)pyridin-3-yl)-3,4-dihydroisoquinolin-1(2H)-one (I-10)

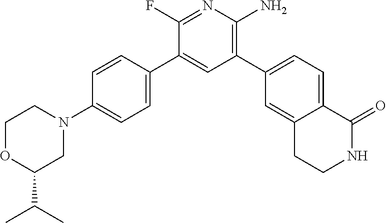 [SMILES: CC(C)[C@H]1CN(c2ccc(-c3cc(-c4ccc5c(c4)CCNC5=O)c(N)nc3F)cc2)CCO1]

A 30 mL vial with magnetic stir bar was charged (S)-3-chloro-6-fluoro-5-(4-(2-isopropylmorpholino)phenyl)pyridin-2-amine (0.028 g, 0.080 mmol), 3,4-dihydro-1(2H)-isoquinolinone-6-boronic acid pinacol ester (0.033 g, 0.120 mmol) and XPhos Pd G2 (6.30 mg, 8.00 μmol). The vial was sealed with a cap and septum and the reaction vessel was evacuated and backfilled with nitrogen. 1,4-Dioxane (1.5 ml) and aqueous potassium phosphate (0.2 ml) were added and the reaction vessel was evacuated and backfilled with nitrogen an additional time. The reaction mixture was heated at 85° C. for 18 h in an aluminum block. The reaction mixture was concentrated onto celite and purified by flash chromatography [0.5-9.5% MeOH/DCM+0.5% NH4OH] to afford the title compound (26 mg, 70.5% yield). 1H NMR (500 MHz, DMSO-d6) δ 7.9-8.0 (m, 2H), 7.58 (d, 1H, J=10.1 Hz), 7.4-7.5 (m, 4H), 7.01 (br d, 2H, J=8.7 Hz), 6.18 (s, 2H), 3.9-4.0 (m, 1H), 3.6-3.6 (m, 3H), 3.53 (br d, 2H, J=12.1 Hz), 3.40 (br dd, 5H, J=2.2, 6.1 Hz), 3.2-3.3 (m, 2H), 2.95 (br t, 2H, J=6.2 Hz), 2.66 (dt, 1H, J=2.9,11.8 Hz), 2.44 (br t, 1H, J=11.2 Hz), 1.73 (qd, 1H, J=6.6, 13.4 Hz), 0.96 (dd, 6H, J=4.3, 6.7 Hz); LCMS: [M+H]⁺=461.47.

### Example 11: (R)-6-(2-amino-6-fluoro-5-(2-fluoro-4-(2-isopropylmorpholino)phenyl)pyridin-3-yl)-3,4-dihydroisoquinolin-1(2H)-one (I-11)

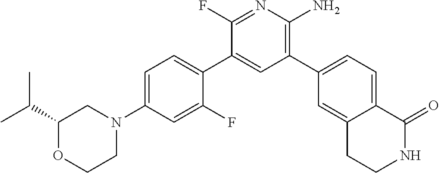 [SMILES: CC(C)[C@@H]1CN(c2ccc(-c3cc(-c4ccc5c(c4)CCNC5=O)c(N)nc3F)c(F)c2)CCO1]

Step 1: (R)-3-chloro-6-fluoro-5-(2-fluoro-4-(2-isopropylmorpholino)phenyl)pyridin-2-amine

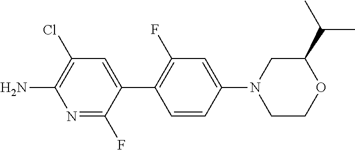 [SMILES: CC(C)[C@@H]1CN(c2ccc(-c3cc(Cl)c(N)nc3F)c(F)c2)CCO1]

A 30 mL vial was charged with 5-bromo-3-chloro-6-fluoropyridin-2-amine (0.040 g, 0.18 mmol, prepared according to Example 8), (R)-4-(3-fluoro-4-(4,4,5,5-tetramethyl-1,3,2-dioxaborolan-2-yl)phenyl)-2-isopropylmorpholine (0.071 g, 0.20 mmol) and Pd(dppf)Cl₂ (0.014 g, 0.018 mmol). The vial was sealed with a cap and septum and the reaction vial was evacuated and backfilled with nitrogen. 1,4-Dioxane (2 mL) and 2 M aqueous Na₂CO₃ (0.27 mL, 0.53 mmol) were added and the reaction vial was evacuated and backfilled with nitrogen an additional time. The reaction was heated at 85° C. for 18 h. After cooling to RT the reaction mixture was concentrated directly onto Celite® and purified by silica gel chromatography (eluting with 5-95% EtOAc/hexanes) to afford the product (0.039 g, 60% yield). LCMS: [M+H]⁺=368.3.

Step 2: (R)-6-(2-amino-6-fluoro-5-(2-fluoro-4-(2-isopropylmorpholino)phenyl)pyridin-3-yl)-3,4-dihydroisoquinolin-1(2H)-one

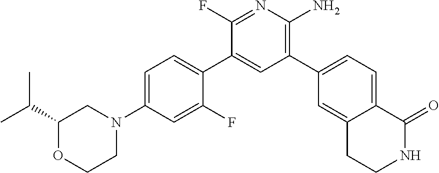 [SMILES: CC(C)[C@@H]1CN(c2ccc(-c3cc(-c4ccc5c(c4)CCNC5=O)c(N)nc3F)c(F)c2)CCO1]

A 30 mL vial was charged with (R)-3-chloro-6-fluoro-5-(2-fluoro-4-(2-isopropylmorpholino)phenyl)pyridin-2-amine (0.039 g, 0.11 mmol), 3,4-dihydro-1(2H)-isoquinolinone-6-boronic acid pinacol ester (0.043 g, 0.16 mmol) and XPhos Pd G2 (8 mg, 10 μmol). The vial was sealed with a cap and septum and the reaction vial was evacuated and backfilled with nitrogen. 1,4-Dioxane (1.5 mL) and 1.3 M aqueous K₃PO₄ (0.20 mL of a 1.3 M solution, 0.27 mmol) were added and the reaction vial was evacuated and backfilled with nitrogen an additional time. The reaction was heated at 90° C. for 18 h. After cooling to RT the reaction mixture was concentrated onto Celite® and purified by silica gel chromatography (eluting with 0.5-9.5% MeOH/DCM+0.5% NH₄OH) to afford the title compound (33 mg, 65%). ¹H NMR (500 MHz, DMSO-d₆) δ 7.9-8.0 (m, 2H), 7.4-7.5 (m, 3H), 7.29 (br t, J=8.8 Hz, 1H), 6.8-6.9 (m, 2H), 6.27 (br s, 2H), 3.96 (br d, J=10.0 Hz, 1H), 3.5-3.6 (m, 4H), 3.40 (br s, 3H), 3.2-3.3 (m, 2H), 2.94 (br t, J=6.0 Hz, 2H), 2.7-2.7 (m, 1H), 1.7-1.8 (m, 1H), 0.9-1.0 (m, 6H); LCMS: [M+H]⁺=479.4.

### Example 12: (S)-6-(2-amino-6-fluoro-5-(2-fluoro-4-(2-isopropylmorpholino)phenyl)pyridin-3-yl)-3,4-dihydroisoquinolin-1(2H)-one (I-12)

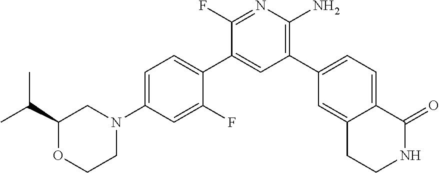 [SMILES: CC(C)[C@H]1CN(c2ccc(-c3cc(-c4ccc5c(c4)CCNC5=O)c(N)nc3F)c(F)c2)CCO1]

Step 1: (S)-3-chloro-6-fluoro-5-(2-fluoro-4-(2-isopropylmorpholino)phenyl)pyridin-2-amine

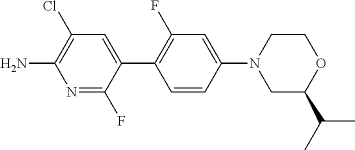 [SMILES: CC(C)[C@H]1CN(c2ccc(-c3cc(Cl)c(N)nc3F)c(F)c2)CCO1]

A 30 mL vial was charged with 5-bromo-3-chloro-6-fluoropyridin-2-amine (0.040 g, 0.18 mmol, prepared according to Example 8), (S)-4-(3-fluoro-4-(4,4,5,5-tetramethyl-1,3,2-dioxaborolan-2-yl)phenyl)-2-isopropylmorpholine (0.071 g, 0.20 mmol) and [1,12-bis(diphenylphosphino)ferrocene]dichloropalladium(II), DCM complex (0.014 g, 0.018 mmol). The vial was sealed with a cap and septum and the reaction vial was evacuated and backfilled with nitrogen. 1,4-Dioxane (2 mL) and 2 M aqueous Na₂CO₃ (0.27 mL, 0.53 mmol) were added and the reaction vial was evacuated and backfilled with N₂ an additional time. The reaction was heated at 85° C. for 18 h. After cooling to RT the reaction mixture was concentrated onto Celite® and purified by silica gel chromatography (eluting with 5-95% EtOAc/hexanes) to afford the desired product (0.036 g, 55%). LCMS: [M+H]⁺=368.3.

Step 2: (S)-6-(2-amino-6-fluoro-5-(2-fluoro-4-(2-isopropylmorpholino)phenyl)pyridin-3-yl)-3,4-dihydroisoquinolin-1(2H)-one

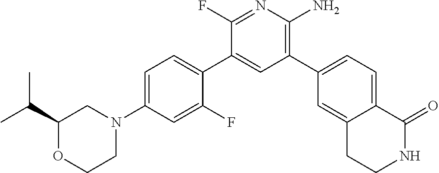 [SMILES: CC(C)[C@H]1CN(c2ccc(-c3cc(-c4ccc5c(c4)CCNC5=O)c(N)nc3F)c(F)c2)CCO1]

A 30 mL vial was charged with (S)-3-chloro-6-fluoro-5-(2-fluoro-4-(2-isopropylmorpholino)phenyl)pyridin-2-amine (0.036 g, 0.098 mmol), 3,4-dihydro-1(2H)-isoquinolinone-6-boronic acid pinacol ester (0.040 g, 0.15 mmol) and XPhos Pd G2 (8 mg, 10 μmol). The vial was sealed with a cap and septum and the reaction vial was evacuated and backfilled with nitrogen. 1,4-Dioxane (1.5 mL) and 1.3 M aqueous K₃PO₄ (0.19 mL, 0.25 mmol) were added and the reaction vial was evacuated and backfilled with nitrogen an additional time. The reaction was heated at 90° C. for 18 h. After cooling to RT the mixture was concentrated onto Celite® and purified by silica gel chromatography (eluting with 0.5-9.5% MeOH/DCM+0.5% NH₄OH) to afford the title compound (0.029 g, 62%). 1 H NMR (500 MHz, DMSO-d₆) δ 7.9-7.9 (m, 2H), 7.4-7.5 (m, 3H), 7.2-7.3 (m, 1H), 6.8-6.9 (m, 2H), 6.25 (br s, 2H), 3.9-4.0 (m, 1H), 3.5-3.6 (m, 3H), 3.39 (br d, J=3.4 Hz, 3H), 3.2-3.3 (m, 1H), 2.94 (br d, J=5.7 Hz, 2H), 2.7-2.7 (m, 1H), 1.7-1.8 (m, 1H), 0.9-1.0 (m, 6H); LCMS [M+H]⁺=479.4.

### Example 13: 6-(2-amino-6-fluoro-5-(4-((1R,5S)-8-methyl-3,8-diazabicyclo[3.2.1]octan-3-yl)phenyl)pyridin-3-yl)-3,4-dihydroisoquinolin-1(2H)-one (I-13)

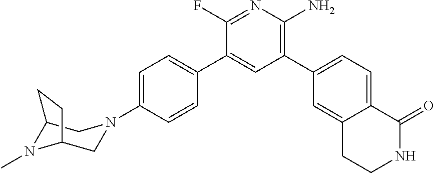 [SMILES: CN1C2CCC1CN(c1ccc(-c3cc(-c4ccc5c(c4)CCNC5=O)c(N)nc3F)cc1)C2]

Step 1: (1R,5S)-3-(4-iodophenyl)-8-methyl-3,8-diazabicyclo[3.2.1]octane

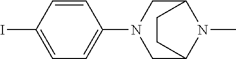 [SMILES: CN1C2CCC1CN(c1ccc(I)cc1)C2]

A sealed mixture of 8-methyl-3,8-diaza-bicyclo[3.2.1]octane dihydrochloride (0.802 g, 4.03 mmol) and K₃PO₄ (2.57 g, 12.09 mmol) in DMF (20 mL) was stirred at RT overnight. The mixture was then degassed with a stream of N₂ before Cul (0.154 g, 0.806 mmol), (R)-(+)-1,1′-bi-2-naphthol (0.231 g, 0.806 mmol) and 1,4-diiodobenzene (1.330 g, 4.03 mmol) were added as solids. The degassing step was repeated and the mixture was sealed and heated while stirring at 40° C. for 2 d. The reaction mixture was cooled to RT, diluted with EtOAc and filtered through Celite®. The filtrate was concentrated under reduced pressure and purified by silica gel chromatography (50 g SiO₂ Biotage® cartridge, using MeOH in CH₂Cl₂ eluting at 19% MeOH) to afford the product as a beige solid (310.0 mg, 23%). LCMS: [M+H]⁺=329.00.

Step 2: 3-chloro-6-fluoro-5-(4-((1R,5S)-8-methyl-3,8-diazabicyclo[3.2.1]octan-3-yl)phenyl)pyridin-2-amine

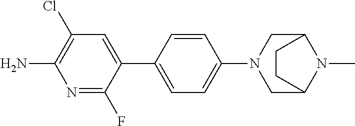 [SMILES: CN1C2CCC1CN(c1ccc(-c3cc(Cl)c(N)nc3F)cc1)C2]

A procedure similar to General Method A (Example 1, Step 2) using (1R,5S)-3-(4-iodophenyl)-8-methyl-3,8-diazabicyclo[3.2.1]octane (99 mg, 0.300 mmol), Cs₂CO₃ (293 mg, 0.901 mmol), PdCl₂dppf (21.97 mg, 0.030 mmol), H₂O (2 mL), 3-chloro-6-fluoro-5-(4,4,5,5-tetramethyl-1,3,2-dioxaborolan-2-yl)pyridin-2-amine (90 mg, 0.330 mmol) in 1,4-dioxane (2.7 mL), followed by purification with silica gel chromatography (25 g SiO₂ Biotage® cartridge, using MeOH in CH₂Cl₂ eluting at 6-90% MeOH) afforded the product as a tan solid (93 mg, 80% yield based on purity of 90%). LCMS: [M+H]⁺=347.35.

Step 3: Synthesis of 6-(2-amino-6-fluoro-5-(4-((1R,5S)-8-methyl-3,8-diazabicyclo[3.2.1]octan-3-yl)phenyl)pyridin-3-yl)-3,4-dihydroisoquinolin-1(2H)-one

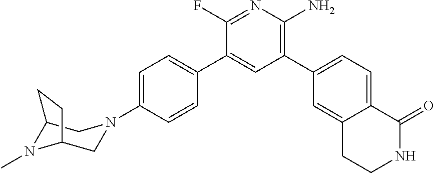 [SMILES: CN1C2CCC1CN(c1ccc(-c3cc(-c4ccc5c(c4)CCNC5=O)c(N)nc3F)cc1)C2]

The title compound was prepared by a method similar to General Method A (Example 1, Step 2) using XPhos Pd G2 (9.51 mg, 0.012 mmol), 6-(4,4,5,5-tetramethyl-1,3,2-dioxaborolan-2-yl)-3,4-dihydroisoquinolin-1(2H)-one (39.6 mg, 0.145 mmol), H₂O (2 mL), 3-chloro-6-fluoro-5-(4-((1 R,5S)-8-methyl-3,8-diazabicyclo[3.2.1]octan-3-yl)phenyl)pyridin-2-amine (46.6 mg, 0.121 mmol) and MeCN (3 mL); by heating under microwave irradiation at 100° C. for 4 h. Purified by chromatography (25 g SiO₂ Biotage® cartridge, using MeOH in CH₂Cl₂ eluting at 30-100% MeOH) followed by filtration through a Waters PoraPak CX column afforded the title compound as a white solid (18.0 mg, 33%). ¹H NMR (500 MHz, DMSO-d₆) δ ppm 7.88-7.96 (m, 2H), 7.56 (br d, J=10.2 Hz, 1H), 7.46 (br d, J=8.1 Hz, 1H), 7.44 (s, 1H), 7.37 (br d, J=8.0 Hz, 2H), 6.82-6.86 (m, 2H), 6.10-6.15 (m, 2H), 3.21 (br s, 2H), 2.93-2.98 (m, 2H), 2.84 (br d, J=9.8 Hz, 2H), 2.22 (s, 3H), 1.91-2.00 (m, 2H), 1.58-1.66 (m, 2H). LCMS: [M+H]⁺=458.46.

### Example 14: 6-(2-amino-6-fluoro-5-(4-((1R,5S)-3-methyl-3-azabicyclo[3.1.0]hexan-1-yl)phenyl)pyridin-3-yl)-3,4-dihydroisoquinolin-1(2H)-one (I-14)

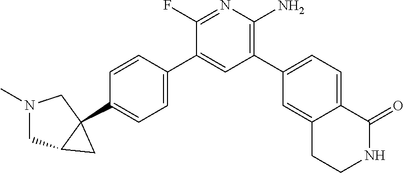 [SMILES: CN1C[C@H]2C[C@@]2(c2ccc(-c3cc(-c4ccc5c(c4)CCNC5=O)c(N)nc3F)cc2)C1]

Step 1: (1R,5S)-1-(4-bromophenyl)-3-methyl-3-azabicyclo[3.1.0]hexan

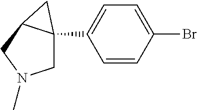

To a solution of (1R,5S)-1-(4-bromophenyl)-3-azabicyclo[3.1.0]hexane (0.500 g, 2.100 mmol) in MeOH/THF (5 mL each) was added formaldehyde solution, 37% wt in water (0.234 mL, 3.15 mmol) followed by NaBH(OAc)₃ (0.668 g, 3.15 mmol). The reaction was stirred at RT for 40 h. The volatiles were removed in vacuo and the residue was partitioned between KOH (1N) and CH₂Cl₂. The layers were separated and the aqueous layer was extracted with additional CH₂Cl₂. The combined extracts were dried over Mg₂SO₄ and concentrated to dryness to afford the product (501 mg, 95%). LCMS: [M+H]⁺=252.21.

Step 2: 3-chloro-6-fluoro-5-(4-((1R,5S)-3-methyl-3-azabicyclo[3.1.0]hexan-1-yl)phenyl)pyridin-2-amine

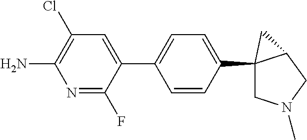 [SMILES: CN1C[C@H]2C[C@@]2(c2ccc(-c3cc(Cl)c(N)nc3F)cc2)C1]

A procedure similar to Example 1, Step 2 (General Method A) using (1R,5S)-1-(4-bromophenyl)-3-methyl-3-azabicyclo[3.1.0]hexane (0.044 g, 0.174 mmol), PdCl₂dppf (0.013 g, 0.017 mmol), Cs₂CO₃ (0.170 g, 0.523 mmol), H₂O (2 mL), 3-chloro-6-fluoro-5-(4,4,5,5-tetramethyl-1,3,2-dioxaborolan-2-yl)pyridin-2-amine (57 mg, 0.21 mmol) in 1,4-dioxane (1.74 mL), DME (2 mL), followed by purification by flash chromatography (25 g SiO₂ Biotage® cartridge, using MeOH in CH₂Cl₂ eluting at 23% MeOH) afforded the product as a brown solid (45 mg, 78% yield based on purity of 96%). LCMS: [M+H]⁺=318.24.

Step 3: 6-(2-amino-6-fluoro-5-(4-((1R,5S)-3-methyl-3-azabicyclo[3.1.0]hexan-1-yl)phenyl)pyridin-3-yl)-3,4-dihydroisoquinolin-1(2H)-one

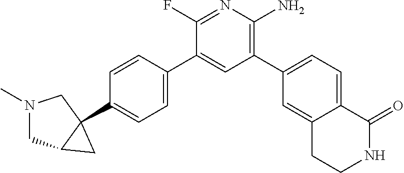 [SMILES: CN1C[C@H]2C[C@@]2(c2ccc(-c3cc(-c4ccc5c(c4)CCNC5=O)c(N)nc3F)cc2)C1]

A procedure similar to Example 1, Step 2 (General Method A) using 6-(4,4,5,5-tetramethyl-1,3,2-dioxaborolan-2-yl)-3,4-dihydroisoquinolin-1(2H)-one(0.040 g, 0.145 mmol), K₃PO₄ (0.092 g, 0.435 mmol), 3-chloro-6-fluoro-5-(4-((1 R,5S)-3-methyl-3-azabicyclo[3.1.0]hexan-1-yl)phenyl)pyridin-2-amine (0.045 g, 0.142 mmol), H₂O (3 mL), MeCN (5 mL), XPhos Pd G2 (0.011 g, 0.015 mmol) and purification by silica gel chromatography (25 g SiO₂ Biotage® cartridge, using MeOH in CH₂Cl₂ eluting at 25% MeOH,), followed by filtration through a Waters PoraPak CX column, afforded the title compound (26 mg, 41% yield based on purity of 98%) as a white solid. ¹H NMR (500 MHz, DMSO-d₆) δ ppm 7.88-7.97 (m, 1H), 7.56-7.66 (m, 1H), 7.41-7.51 (m, 4H), 7.18 (br d, J=8.3 Hz, 2H), 6.26 (s, 2H), 3.20-3.32 (m, 1H), 2.93-3.00 (m, 3H), 2.39 (dd, J=8.6, 3.2 Hz, 1H), 2.30 (s, 3H), 1.75-1.84 (m, 1H), 1.32-1.38 (m, 1H), 0.76 (dd, J=7.8, 3.7 Hz, 1H). Multiplets corresponding to two CH₂ multiplets are obscured by the solvent and/or H₂O peaks. LCMS [M+H]⁺=429.35.

### Example 15: 6-(2-amino-6-fluoro-5-(4-(isopropylsulfonyl)phenyl)pyridin-3-yl)-3,4-dihydroisoquinolin-1(2H)-one (I-15)

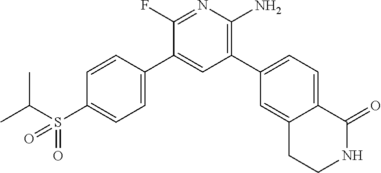 [SMILES: CC(C)S(=O)(=O)c1ccc(-c2cc(-c3ccc4c(c3)CCNC4=O)c(N)nc2F)cc1]

Step 1. 3-chloro-4-fluoro-5-(4-(isopropylsulfonyl)phenyl)pyridin-2-amine

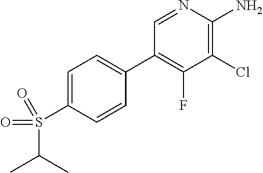 [SMILES: CC(C)S(=O)(=O)c1ccc(-c2cnc(N)c(Cl)c2F)cc1]

In a microwave vial with a magnetic stir bar was placed 5-bromo-3-chloro-6-fluoropyridin-2-amine (260 mg, 1.15 mmol, prepared according to Example 9), [1,1′-bis(diphenylphosphino)ferrocene]dichloropalladium(II) (84 mg, 0.115 mmol), Cs₂CO₃ (1.13 g, 3.46 mmol), 1,2-dimethoxyethane (DME) (4 mL) and H₂O (2 mL). The flask was sealed, then heated to 90° C. for 1 h in the microwave until LCMS indicated complete conversion. The reaction was loaded onto Celite® and purified by reverse phase chromatography (C₁₈ column, eluting with MeCN—H₂O) to give the product as a brown solid (161 mg, 43%). LCMS: [M+H]⁺=329.15.

Step 2: 6-(2-amino-6-fluoro-5-(4-(isopropylsulfonyl)phenyl)pyridin-3-yl)-3,4-dihydroisoquinolin-1(2H)-one

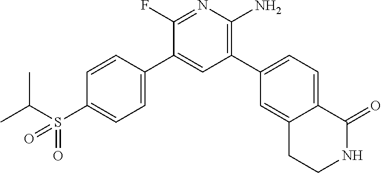 [SMILES: CC(C)S(=O)(=O)c1ccc(-c2cc(-c3ccc4c(c3)CCNC4=O)c(N)nc2F)cc1]

In a microwave vial with magnetic stir bar was placed 3-bromo-6-fluoro-5-(4-(isopropylsulfonyl)phenyl)pyridin-2-amine (53 mg, 0.142 mmol), 3,4-dihydro-1(2H)-isoquinolinone-6-boronic acid pinacol ester (58.2 mg, 0.213 mmol), XPhos Pd G2 (5.59 mg, 7.10 μmol), Cs₂CO₃ (139 mg, 0.426 mmol), DME (4 mL) and H₂O (2 mL). The flask was sealed, then heated to 90° C. for 1 h in the microwave until LCMS indicated complete conversion. The reaction was loaded onto Celite® and purified by reverse phase chromatography (C18, MeCN—H₂O) to give the title compound as a tan solid (49 mg, 78%). ¹H NMR (500 MHz, DMSO-d₆) δ 7.95 (br s, 1H), 7.92 (br d, J=7.8 Hz, 1H), 7.87 (s, 4H), 7.78 (br d, J=10.1 Hz, 1H), 7.49 (br d, J=7.9 Hz, 1H), 7.46 (s, 1H), 6.55 (br s, 2H), 3.47-3.39 (m, 3H), 2.96 (br t, J=6.2 Hz, 2H), 1.18 (d, J=6.8 Hz, 6H); LCMS: [M+H]⁺=440.49.

### Example 16: 6-(2-amino-6-fluoro-5-(4-morpholinophenyl)pyridin-3-yl)-3,4-dihydroisoquinolin-1(2H)-one (I-16)

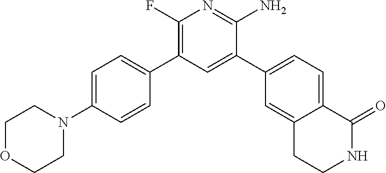 [SMILES: Nc1nc(F)c(-c2ccc(N3CCOCC3)cc2)cc1-c1ccc2c(c1)CCNC2=O]

Step 1: 3-chloro-6-fluoro-5-(4-morpholinophenyl)pyridin-2-amine

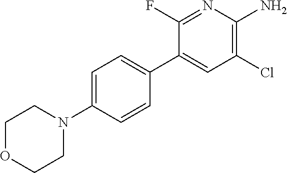 [SMILES: Nc1nc(F)c(-c2ccc(N3CCOCC3)cc2)cc1Cl]

A procedure similar to that of Example 16, Step 1 using 5-bromo-3-chloro-6-fluoropyridin-2-amine (270 mg, 1.20 mmol, prepared according to Example 8), [1,1′-Bis(diphenylphosphino)ferrocene]dichloropalladium(II) (88 mg, 0.120 mmol), Cs₂CO₃ (1.17 g, 3.59 mmol), DME (4 mL) and H₂O (2 mL) afforded the product (422 mg, 92%). LCMS: [M +H]⁺=310.20.

Step 2: 6-(2-amino-6-fluoro-5-(4-morpholinophenyl)pyridin-3-yl)-3,4-dihydroisoquinolin-1(2H)-one

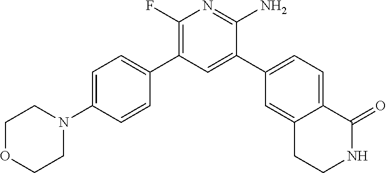 [SMILES: Nc1nc(F)c(-c2ccc(N3CCOCC3)cc2)cc1-c1ccc2c(c1)CCNC2=O]

The procedure was similar to Example 16, Step 2 using 3-bromo-6-fluoro-5-(4-morpholinophenyl)pyridin-2-amine (65 mg, 0.185 mmol), 3,4-dihydro-1(2H)-isoquinolinone-6-boronic acid pinacol ester (76 mg, 0.277 mmol), XPhos Pd G2 (7.26 mg, 9.23 μmol), Cs₂CO₃ (180 mg, 0.554 mmol), DME (4 mL) and H₂O (2 mL). Workup and purification afforded the title compound as a tan solid (18 mg, 23%). ¹H NMR (500 MHz, DMSO-d₆) δ 7.97-7.87 (m, 2H), 7.59 (br d, J=10.1 Hz, 1H), 7.49-7.40 (m, 4H), 6.99 (br d, J=8.7 Hz, 2H), 6.18 (br s, 2H), 3.79-3.70 (m, 4H), 3.40 (br d, J=4.2 Hz, 2H), 3.17-3.10 (m, 4H), 2.95 (br t, J=6.2 Hz, 2H); LCMS: [M+H]⁺=419.31.

### Example 17: 6-(2-amino-6-fluoro-5-(4-(piperazin-1-yl)phenyl)pyridin-3-yl)-3,4-dihydroisoquinolin-1(2H)-one (I-17)

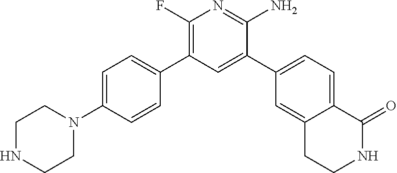 [SMILES: Nc1nc(F)c(-c2ccc(N3CCNCC3)cc2)cc1-c1ccc2c(c1)CCNC2=O]

Step 1: tert-butyl 4-(4-(6-amino-5-chloro-2-fluoropyridin-3-yl)phenyl)piperazine-1-carboxylate

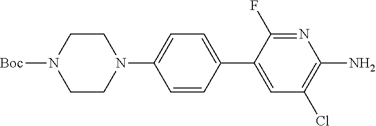 [SMILES: CC(C)(C)OC(=O)N1CCN(c2ccc(-c3cc(Cl)c(N)nc3F)cc2)CC1]

A procedure similar to General Procedure A (Example 1) using 5-bromo-3-chloro-6-fluoropyridin-2-amine (300 mg, 1.331 mmol), Cs₂CO₃ (1301 mg, 3.99 mmol), tert-butyl 4-[4-(4,4,5,5-tetramethyl-1,3,2-dioxaborolan-2-yl)phenyl]tetrahydro-1(2H)-pyrazinecarboxylate (672 mg, 1.73 mmol) and [1,1-bis(diphenylphosphino)ferrocene]dichloropalladium(II) (117 mg, 0.160 mmol) in DME (15 mL) and H₂O (3 mL) afforded the product as a white solid (360 mg, 66%). LCMS: [M+H]⁺=407.46.

Step 2: tert-butyl 4-(4-(6-amino-2-fluoro-5-(1-oxo-1,2,3,4-tetrahydroisoquinolin-6-yl)pyridin-3-yl)phenyl)piperazine-1-carboxylate

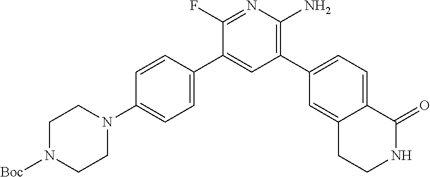 [SMILES: CC(C)(C)OC(=O)N1CCN(c2ccc(-c3cc(-c4ccc5c(c4)CCNC5=O)c(N)nc3F)cc2)CC1]

A procedure similar to that of Example 1, General Method A using XPhos Pd G2 (30.2 mg, 0.038 mmol) 3,4-dihydro-1(2H)-isoquinolinone-6-boronic acid pinacol ester (105 mg, 0.383 mmol), tert-butyl 4-(4-(6-amino-5-chloro-2-fluoropyridin-3-yl)phenyl)piperazine-1-carboxylate (120 mg, 0.295 mmol) and Cs₂CO₃ (288 mg, 0.885 mmol) in a solvent mixture of DME (10 mL) and H₂O (2.5 mL) flushed with argon gas. The mixture was heated at 90° C. in a microwave reactor for 1 h. Standard workup followed by purification using silica gel chromatography (eluting with hexanes/EtOAc followed by 3% MeOH and 0.3% NH₄OH in DCM) afforded the desired product as an off white foam (106 mg, judged to be 73% pure), which was taken into the next step without further purification.

Step 3. 6-(2-amino-6-fluoro-5-(4-(piperazin-1-yl)phenyl)pyridin-3-yl)-3,4-dihydroisoquinolin-1(2H)-one trifluoroacetic acid salt

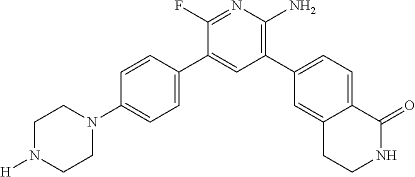 [SMILES: Nc1nc(F)c(-c2ccc(N3CCNCC3)cc2)cc1-c1ccc2c(c1)CCNC2=O]

The tert-butyl 4-(4-(6-amino-2-fluoro-5-(1-oxo-1,2,3,4-tetrahydroisoquinolin-6-yl)pyridin-3-yl)phenyl)piperazine-1-carboxylate (106 mg, 0.149 mmol) obtained in Step 2 was stirred at RT with a 1:1 mixture of DCM (1.5 ml) and TFA (1 mL) for 10 min. Complete conversion to the desired product was observed. The mixture was concentrated to dryness, passed through a porapak cartridge and purified by reverse phase chromatography (5.5 g column, eluting with water containing 0-10% acetonitrile). The appropriate fractions were combined and concentrated to afford the title compound (62.5 mg, 75% yield) as a beige solid. ¹H NMR (500 MHz, DMSO-d₆) δ=8.80-8.56 (m, 1H), 8.01-7.85 (m, 2H), 7.67-7.56 (m, 1H), 7.51-7.38 (m, 4H), 7.09-6.99 (m, 2H), 6.27-6.16 (m, 2H), 3.43-3.37 (m, 6H), 3.26-3.20 (m, 4H), 2.99-2.91 (m, 2H)¹⁹F NMR (471 MHz, DMSO-d₆) 5=−73.45 (s, 1F),−73.47 (s, 1F),−75.83 (s, 1F).

### Example 18: 6-(2-amino-6-fluoro-5-(4-(5-methyl-2,5-diazabicyclo[2.2.1]heptan-2-yl)phenyl)pyridin-3-yl)-3,4-dihydroisoquinolin-1(2H)-one (I-18)

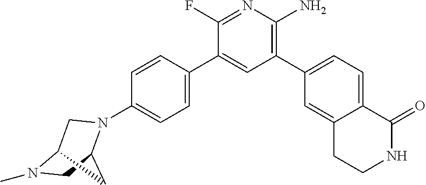 [SMILES: CN1C[C@@H]2C[C@H]1CN2c1ccc(-c2cc(-c3ccc4c(c3)CCNC4=O)c(N)nc2F)cc1]

Step 1: (1S,4S)-2-methyl-5-(4-(4,4,5,5-tetramethyl-1,3,2-dioxaborolan-2-yl)phenyl)-2,5-diazabicyclo[2.2.1]heptane

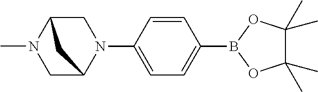

A vial charged with XPhos Pd G2 (21.20 mg, 0.027 mmol), B₂pin₂ (68.4 mg, 0.269 mmol, KOAc (79 mg, 0.808 mmol, (1S,4S)-2-(4-chlorophenyl)-5-methyl-2,5-diazabicyclo[2.2.1]heptane (60 mg, 0.269 mmol and 1,4-dioxane (6 mL). The vial was sealed and degassed by repeated evacuation-refill with N₂. The reaction mixture was heated in an oil bath at 100° C. overnight to afford a crude solution of the product that was used as the crude solution in the subsequent step. LCMS: [M+H]⁺=315.16.

Step 2: 3-chloro-6-fluoro-5-(4-((1S,4S)-5-methyl-2,5-diazabicyclo[2.2.1]heptan-2-yl)phenyl)pyridin-2-amine

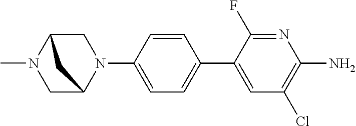 [SMILES: CN1C[C@@H]2C[C@H]1CN2c1ccc(-c2cc(Cl)c(N)nc2F)cc1]

A procedure similar to General Method A in Example 1, Step 2 using 5-bromo-3-chloro-6-fluoropyridin-2-amine (61 mg, 0.27 mmol, prepared according to Example 9), Cs₂CO₃ (264 mg, 0.811 mmol), PdCl₂dppf (19.79 mg, 0.027 mmol), H₂O (2 mL), (1S,4S)-2-methyl-5-(4-(4,4,5,5-tetramethyl-1,3,2-dioxaborolan-2-yl)phenyl)-2,5-diazabicyclo[2.2.1]heptane (85 mg, 0.270 mmol) in 1,4-dioxane (6 mL), DME (1.3 mL) afforded after workup and purification the product as a tan solid (113.0 mg, quantitative yield based on purity of 84%). LCMS: [M+H]⁺=333.50.

Step 3: 6-(2-amino-6-fluoro-5-(4-(5-methyl-2,5-diazabicyclo[2.2.1]heptan-2-yl)phenyl)pyridin-3-yl)-3,4-dihydroisoquinolin-1(2H)-one

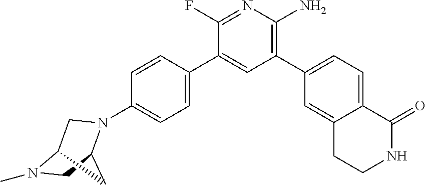 [SMILES: CN1C[C@@H]2C[C@H]1CN2c1ccc(-c2cc(-c3ccc4c(c3)CCNC4=O)c(N)nc2F)cc1]

A procedure similar to General Method A (Example 1, Step 2) using 3-chloro-6-fluoro-5-(4-((1 S,4S)-5-methyl-2,5-diazabicyclo[2.2.1]heptan-2-yl)phenyl)pyridin-2-amine (0.113 g, 0.285 mmol), K₃PO₄ (0.181 g, 0.855 mmol), 6-(4,4,5,5-tetramethyl-1,3,2-dioxaborolan-2-yl)-3,4-dihydroisoquinolin-1(2H)-one (0.086 g, 0.314 mmol), H₂O (6 mL), MeCN (9 mL) and XPhos Pd G2 (0.022 g, 0.0285 mmol) afforded the title compound as a white solid (25 mg, 20%). ¹H NMR (500 MHz, DMSO-d₆): δ ppm 7.86 (br s, 1H), 7.83 (d, J=8.0 Hz, 1H), 7.49 (d, J=10.2 Hz, 1H), 7.39 (br d, J=8.0 Hz, 1H), 7.37 (s, 1H), 7.27 (br d, J=8.0 Hz, 2H), 6.54 (br d, J=8.7 Hz, 1H), 6.04 (s, 2H), 4.24 (s, 1H), 3.30-3.40 (m, 3H), 3.06-3.14 (m, 1H), 2.83-2.91 (m, 2H), 2.67-2.77 (m, 1H), 2.19 (s, 3H), 1.76-1.83 (m, 1H), 1.68-1.72 (m, 1H). LCMS: [M+H]⁺=444.32.

### Example 19: 6-(2-amino-6-fluoro-5-(4-morpholinophenyl)pyridin-3-yl)-7-fluoro-3,4-dihydroisoquinolin-1(2H)-one (I-19)

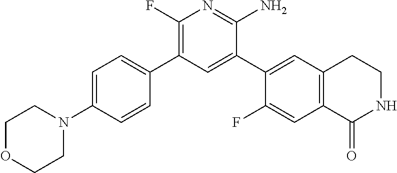 [SMILES: Nc1nc(F)c(-c2ccc(N3CCOCC3)cc2)cc1-c1cc2c(cc1F)C(=O)NCC2]

Step 1: 6-bromo-7-fluoro-3,4-dihydroisoquinolin-1(2H)-one

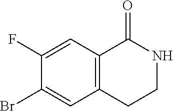 [SMILES: O=C1NCCc2cc(Br)c(F)cc21]

Sodium azide (114 mg, 1.75 mmol, 2 eq.) was added to a solution of 5-bromo-6-fluoro-2,3-dihydro-1H-inden-1-one (200 mg, 0.873 mmol, 1 eq) in 0.4 mL of mixture of methane sulfonic acid and CH₂Cl₂ (1:1) in portionwise at 0° C. The resulting mixture was stirred for 8 h at RT. The reaction mixture was cooled to 0° C. in ice bath, neutralized with 5% aq. NaOH and aqueous layer was extracted with EtOAc (2×10 mL). The combined organic layers were washed with H₂O and brine solution, dried over MgSO₄ and filtered. The filtrate was concentrated under vacuum and purified by silica gel flash column chromatography using CH₂Cl₂-MeOH to obtain the product (150 mg, 70%). LCMS: [M+H]⁺=246.20 Step 2: 8-fluoro-6-(4,4,5,5-tetramethyl-1,3,2-dioxaborolan-2-yl)-3,4-dihydroiso-quinolin-1(2H)-one

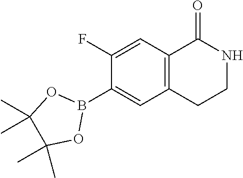 [SMILES: CC1(C)OB(c2cc3c(cc2F)C(=O)NCC3)OC1(C)C]

To 6-bromo-5-fluoro-3,4-dihydroisoquinolin-1(2H)-one (50 mg, 0.205 mmol, 1eq), bis(pinacolato)diboron (58.2 mg, 0.229 mmol, 1.1 eq), K₂CO₃ (61.3 mg, 0.625 mmol, 3 eq) and [1,12-bis(diphenylphosphino)ferrocene]dichloropalladium(II) (7.62 mg, 10.41 μmol, 0.05 eq) in 1,4-dioxane (2.5 mL) was added under Ar. The mixture was heated in a microwave for 2 h at 100° C. The LCMS showed less than 10% of starting material. The reaction mixture was used in the next steps without purification. LCMS: [M+H]⁺=292.0

Step 3: 3-chloro-6-fluoro-5-(4-morpholinophenyl)pyridin-2-amine

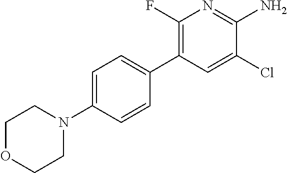 [SMILES: Nc1nc(F)c(-c2ccc(N3CCOCC3)cc2)cc1Cl]

XPhos Pd G2 (20.39 mg, 0.026 mmol), 4-(morpholino)phenylboronic acid (248 mg, 1.198 mmol), 5-bromo-3-chloro-6-fluoropyridin-2-amine (270 mg, 2.0 mmol, preparation described in Example 9), and acetonitrile (3 mL) were added to a vial. K₃PO₄ (165 mg, 0.778 mmol) in H₂O (2 mL) was added to the reaction mixture. The reaction was degassed with N₂ and heated sealed in a microwave reactor at 100° C. for 4 h. The reaction mixture was worked up and purified in a manner similar to previous examples to afford the desired product (280 mg, 72%). LCMS: [M+H]⁺=308.24.

Step 4: 6-(2-amino-6-fluoro-5-(4-morpholinophenyl)pyridin-3-yl)-7-fluoro-3,4-dihydroisoquinolin-1(2H)-one

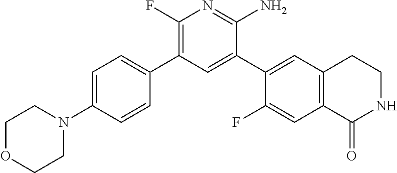 [SMILES: Nc1nc(F)c(-c2ccc(N3CCOCC3)cc2)cc1-c1cc2c(cc1F)C(=O)NCC2]

A small vial was charged with XPhos Pd G2 (20.4 mg, 0.026 mmol) and 7-fluoro-6-(4,4,5,5-tetramethyl-1,3,2-dioxaborolan-2-yl)-3,4-dihydroisoquinolin-1(2H)-one (83 mg, 0.285 mmol) and in acetonitrile (3 mL). A solution of K₃PO₄ (165 mg, 0.778 mmol) in water (2 mL) was added to the reaction mixture. The reaction was degassed with N₂ and heated in a sealed microwave reactor at 100° C. for 4 h. The mixture was then evaporated with Celite® and purified in a manner similar to previous examples to afford the title compound (19 mg, 16%). ¹H NMR (500 MHz, DMSO-d₆) δ 8.10 (br s, 1H), 7.60 (t, J=9.5 Hz, 2H), 7.45-7.38 (m, 3H), 6.98 (br d, J=8.8 Hz, 2H), 6.21-6.16 (m, 2H), 3.78-3.73 (m, 4H), 3.43-3.34 (m, 2H), 3.17-3.10 (m, 4H), 2.92 (br t, J=6.4 Hz, 2H), 2.64-2.62 (m, 1H), 2.46-2.38 (m, 1H), 2.37-2.35 (m, 1H); LCMS: [M+H]⁺=437.32.

### Example 20: 6-(2-amino-6-fluoro-5-(4-(4-(2-fluoroethyl)piperazin-1-yl)phenyl)pyridin-3-yl)-3,4-dihydroisoquinolin-1(2H)-one (I-20)

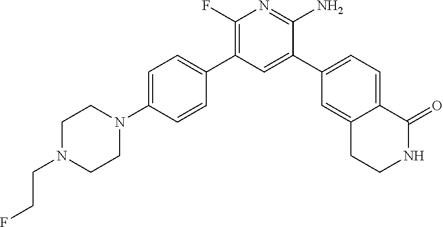 [SMILES: Nc1nc(F)c(-c2ccc(N3CCN(CCF)CC3)cc2)cc1-c1ccc2c(c1)CCNC2=O]

To a slurry of 6-(2-amino-6-fluoro-5-(4-(piperazin-1-yl)phenyl)pyridin-3-yl)-3,4-dihydroisoquinolin-1(2H)-one (30 mg, 0.072 mmol, preparation described in Example 17) in DMF (2 mL) was added 2-fluoroethyl 4-methylbenzenesulfonate (23.52 mg, 0.108 mmol) and Et₃N (0.100 ml, 0.719 mmol) at RT, and the mixture was heated in an oil bath at 100° C. for 12 h. The mixture was allowed to cool to RT, concentrated onto Celite, and purified on a reverse phase column eluting with water containing 0-35% acetonitrile. The material further purified by normal phase silica gel chromatography, eluting with DCM containing 0-2.5% MeOH and 0-0.25% NH₄OH yielded product as a white powder (9 mg, 26%). ¹H NMR (500 MHz, DMSO-d₆) δ 7.8-8.0 (m, 2H), 7.6-7.7 (m, 1H), 7.4-7.5 (m, 2H), 7.41 (br d, J=8.1 Hz, 2H), 6.9-7.1 (m, 2H), 6.1-6.3 (m, 2H), 4.6-4.7 (m, 1H), 4.5-4.6 (m, 1H), 3.4-3.5 (m, 2H), 3.1-3.2 (m, 4H), 2.9-3.0 (m, 2H), 2.6-2.7 (m, 2H), 2.6-2.6 (m, 4H); LCMS: [M+H]⁺=464.47.

### Example 21: 6-(2-amino-5-(4-(4-(2,2-difluoroethyl)piperazin-1-yl)phenyl)-6-fluoropyridin-3-yl)-3,4-dihydroisoquinolin-1(2H)-one (I-21)

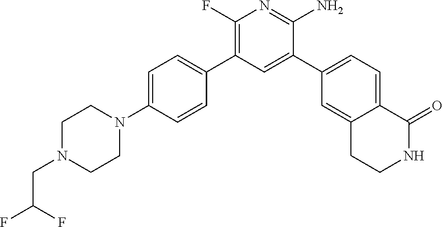 [SMILES: Nc1nc(F)c(-c2ccc(N3CCN(CC(F)F)CC3)cc2)cc1-c1ccc2c(c1)CCNC2=O]

The procedure was similar to that of Example 20 using 6-(2-amino-6-fluoro-5-(4-(piperazin-1-yl)phenyl)pyridin-3-yl)-3,4-dihydroisoquinolin-1(2H)-one (30 mg, 0.072 mmol) and Et₃N (0.100 ml, 0.719 mmol) in DMF (2 mL) at RT, with addition 2,2-difluoroethyl 4-methylbenzenesulfonate (25.5 mg, 0.108 mmol) instead of 2-fluoroethyl 4-methylbenzenesulfonate. The product (10.5 mg, 29%) was isolated as a white powder. ¹H NMR (500 MHz, DMSO-d₆) δ 7.9-8.0 (m, 2H), 7.5-7.6 (m, 1H), 7.4-7.5 (m, 2H), 7.4-7.4 (m, 2H), 6.9-7.1 (m, 2H), 6.0-6.3 (m, 3H), 3.4-3.5 (m, 2H), 3.1-3.2 (m, 4H), 2.9-3.0 (m, 2H), 2.7-2.9 (m, 2H), 2.7-2.7 (m, 4H); LCMS: [M+H]⁺=482.52.

### Example 22: 6-(2-amino-6-fluoro-5-(4-(piperidin-4-yloxy)phenyl)pyridin-3-yl)-3,4-dihydroisoquinolin-1(2H)-one (I-22)

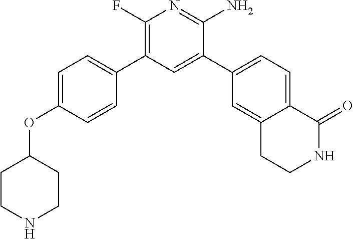 [SMILES: Nc1nc(F)c(-c2ccc(OC3CCNCC3)cc2)cc1-c1ccc2c(c1)CCNC2=O]

Step 1: tert-butyl 4-(4-(6-amino-5-chloro-2-fluoropyridin-3-yl)phenoxy)piperidine-1-carboxylate

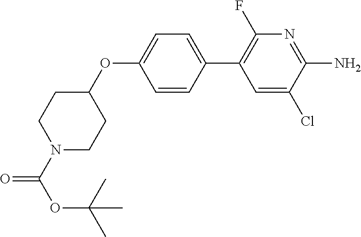 [SMILES: CC(C)(C)OC(=O)N1CCC(Oc2ccc(-c3cc(Cl)c(N)nc3F)cc2)CC1]

To a stirred solution of 5-bromo-3-chloro-6-fluoropyridin-2-amine (100 mg, 0.444 mmol, 1.00 eq), tert-butyl 4-[4-(tetramethyl-1,3,2-dioxaborolan-2-yl)phenoxy]piperidine-1-carboxylate (192 mg, 0.476 mmol, 1.07 eq) in 1,4-Dioxane (5 ml), was added a solution of Cesium carbonate (434 mg, 1.331 mmol, 3.00 eq) in Water (1 ml) and the resulted reaction mixture was degassed under argon for 15 minutes. Then PddppfCl2 (48.7 mg, 0.067 mmol, 0.15 eq) was added and the reaction mixture was heated at 90° C. for overnight. The reaction mixture was dry loaded onto celite and purified by Flash [0-20% DCM/MeOH] to afford the product (187 mg, 100% yield) as a dark beige powder which will be used for the next step. [M+H]+ 422.35.

Step 2: tert-butyl 4-(4-(6-amino-2-fluoro-5-(1-oxo-1,2,3,4-tetrahydroisoquinolin-6-yl)pyridin-3-yl)phenoxy)piperidine-1-carboxylate

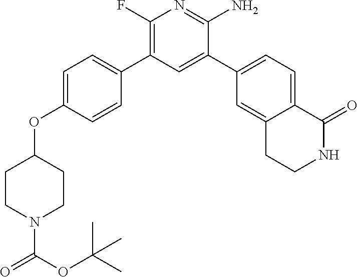 [SMILES: CC(C)(C)OC(=O)N1CCC(Oc2ccc(-c3cc(-c4ccc5c(c4)CCNC5=O)c(N)nc3F)cc2)CC1]

To a stirred solution of 4-(4-(6-amino-5-chloro-2-fluoropyridin-3-yl)phenoxy)piperidine-1-carboxylate (187 mg, 0.443 mmol, 1.00 eq), 3,4-dihydro-1(2H)-isoquinolinone-6-boronic acid pinacol ester (182 mg, 0.665 mmol, 1.50 eq) in 1,4-dioxane (5 ml), was added a solution of potassium phosphate tribasic (282 mg, 1.330 mmol, 3.00 eq) in Water (1 ml) and the resulted mixture was degassed under argon for 15 minutes. Then XPhos Pd G2 (34.9 mg, 0.044 mmol, 0.10 eq) was added and the reaction mixture was heated at 90° C. for 1 hour under microwave condition. The reaction mixture was then dry loaded onto celite and purified by Flash chromatography [0-10% DCM/MeOH] to afford the product (199 mg, 84% yield) as a dark beige powder which will be used for the next step. LCMS: [M+H]⁺=533.33.

Step 3: 6-(2-amino-6-fluoro-5-(4-(piperidin-4-yloxy)phenyl)pyridin-3-yl)-3,4-dihydroisoquinolin-1(2H)-one

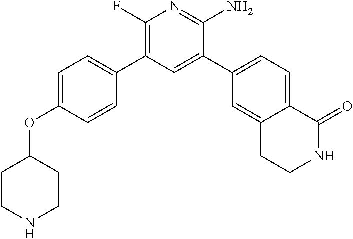 [SMILES: Nc1nc(F)c(-c2ccc(OC3CCNCC3)cc2)cc1-c1ccc2c(c1)CCNC2=O]

To a solution of tert-butyl 4-(4-(6-amino-2-fluoro-5-(1-oxo-1,2,3,4-tetrahydroisoquinolin-6-yl)pyridin-3-yl)phenoxy)piperidine-1-carboxylate (199 mg, 1 eq) in CH₂Cl₂ (5 ml), was added TFA (2 ml, 69.79 eq). The resulted reaction mixture was stirred for 1 hour until completion of the reaction. The solvents were removed and the crude mixture was dissolved in MeOH and passed through a PoraPak Rxn CX (20cc-2g) cartridge in a catch & elute method. The cartridge was washed with MeOH (20 mL) and then the solution of product in MeOH was added onto the cartridge. The cartridge was rinsed with MeOH (3×20 mL) followed by 1× with a solution of 10 mL (NH3 in MeOH @ 7N) in 10 mL of MeOH to release the title compound (124.8 mg, 73.4% yield) as a white powder. ¹H NMR (500 MHz, DMSO-d₆) δ=8.00-7.86 (m, 2H), 7.59 (br d, J=10.1 Hz, 1H), 7.50-7.40 (m, 4H), 6.98 (br d, J=8.7 Hz, 2H), 6.21 (s, 2H), 4.47-4.34 (m, 1H), 3.40 (br d, J=3.9 Hz, 2H), 3.01-2.89 (m, 4H), 2.57 (br s, 3H), 1.91 (br d, J=9.7 Hz, 2H), 1.45 (br d, J=9.5 Hz, 2H); LCMS: [M+H]⁺=433.33.

### Example 23: 6-(2-amino-6-fluoro-5-(4-(hexahydropyrazino[2,1-c][1,4]oxazin-8(1H)-yl)phenyl)pyridin-3-yl)-3,4-dihydroisoquinolin-1(2H)-one (I-23)

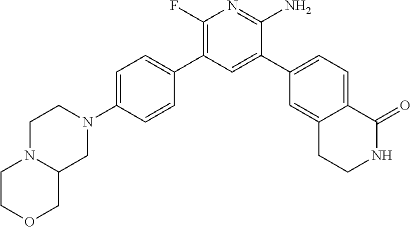 [SMILES: Nc1nc(F)c(-c2ccc(N3CCN4CCOCC4C3)cc2)cc1-c1ccc2c(c1)CCNC2=O]

### Step 1: 8-(4-Iodophenyl)octahydropyrazino[2,1-c][1,4]oxazine

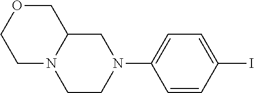 [SMILES: Ic1ccc(N2CCN3CCOCC3C2)cc1]

A sealed mixture of octahydropyrazino[2,1-c][1,4]oxazine (250 mg, 1.758 mmol), K₃PO₄ (1119 mg, 5.27 mmol) and 1,4-diiodobenzene (580 mg, 1.758 mmol) in DMF (10 mL) was degassed with a stream of N₂. Later Cul (67.0 mg, 0.352 mmol) and (R)-(+)-1,1′-bi-2-naphthol (101 mg, 0.352 mmol) were added as solids. The degassing step was repeated. The mixture was then stirred sealed and heated at 40° C. for 2 d. The reaction mixture was cooled to RT, diluted with EtOAc and filtered through Celite®. The filtrate was concentrated under reduced pressure and purified by chromatography (50 g SiO₂ Biotage@cartridge, using MeOH in CH₂Cl₂ eluting at 9% MeOH) to afford the product as a beige solid (310.0 mg, 79%). LCMS: [M+H]⁺=345.41.

Step 2: 3-chloro-6-fluoro-5-(4-(hexahydropyrazino[2,1-c][1,4]oxazin-8(1H)-yl)phenyl)pyridin-2-amine

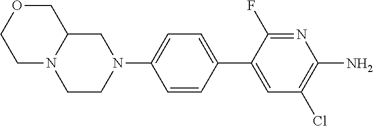 [SMILES: Nc1nc(F)c(-c2ccc(N3CCN4CCOCC4C3)cc2)cc1Cl]

A procedure similar to that described in Example 1, General Method A using 8-(4-iodophenyl)octahydropyrazino[2,1-c][1,4]oxazine (108 mg, 0.314 mmol), PdCl₂dppf (22.95 mg, 0.031 mmol), Cs₂CO₃ (307 mg, 0.941 mmol), H₂O (2 mL), DME (1.8 mL) and 3-chloro-6-fluoro-5-(4,4,5,5-tetramethyl-1,3,2-dioxaborolan-2-yl)pyridin-2-amine (94 mg, 0.345 mmol) in 1,4-dioxane (2.6 mL) and heating under microwave irradiation at 90° C. for 1 h afforded product as an brown solid (127.0 mg, 77% yield based on purity of 69%). LCMS: [M+H]⁺=363.22.

Step 3: 6-(2-amino-6-fluoro-5-(4-(hexahydropyrazino[2,1-c][1,4]oxazin-8(1H)-yl)phenyl)pyridin-3-yl)-3,4-dihydroisoquinolin-1(2H)-one

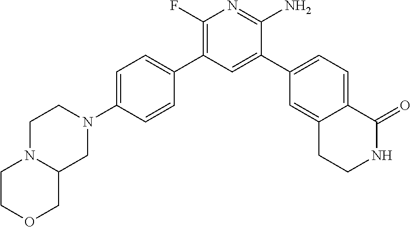 [SMILES: Nc1nc(F)c(-c2ccc(N3CCN4CCOCC4C3)cc2)cc1-c1ccc2c(c1)CCNC2=O]

A procedure similar to that described in Example 1, General Method A using 6-(4,4,5,5-tetramethyl-1,3,2-dioxaborolan-2-yl)-3,4-dihydroisoquinolin-1(2H)-one (0.103 g, 0.377 mmol), K₃PO₄ (0.200 g, 0.942 mmol), 3-chloro-6-fluoro-5-(4-(hexahydropyrazino[2,1-c][1,4]oxazin-8(1H)-yl)phenyl)pyridin-2-amine (0.114 g, 0.314 mmol), and XPhos Pd G2 (0.025 g, 0.031 mmol) in H₂O (9 mL) in CH₃CN (12 mL), and afforded, after purification by flash chromatography (25 g SiO₂ Biotage® cartridge, using MeOH in CH₂Cl₂ eluting at 9% MeOH) and preparative HPLC (30 g Biotage® SNAP KP-C₁₈-HS, MeOH in (H₂O+0.05% TFA) eluting at 38% MeOH) the TFA salt of the title compound as a pale yellow solid (34.0 mg, 18%) ¹H NMR (500 MHz, CD₃OD) δ 8.03 (d, J=8.0 Hz, 1H), 7.59 (d, J=9.8 Hz, 1H), 7.47 (br d, J=8.1 Hz, 3H), 7.44 (s, 1H), 7.08 (brd, J=8.7 Hz, 2H), 4.14 (br d, J=9.2 Hz, 2H), 3.83-3.99 (m, 3H), 3.58-3.68 (m, 3H), 3.46-3.57 (m, 3H), 3.33-3.45 (m, 2H), 3.12-3.21 (m, 1H), 3.01-3.07 (m, 2H), 2.79-2.89 (m, 1H); LCMS: [M+H]⁺=474.31.

### Example 24: (S)-6-(2-amino-6-fluoro-5-(4-(4-(2-methoxypropyl)piperazin-1-yl)phenyl)pyridin-3-yl)-3,4-dihydroisoquinolin-1(2H)-one (I-24)

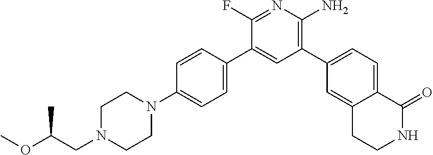 [SMILES: CO[C@@H](C)CN1CCN(c2ccc(-c3cc(-c4ccc5c(c4)CCNC5=O)c(N)nc3F)cc2)CC1]

Step 1: (S)-1-(4-(4-iodophenyl)piperazin-1-yl)-2-methoxypropan-1-one

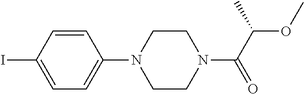 [SMILES: CO[C@@H](C)C(=O)N1CCN(c2ccc(I)cc2)CC1]

To a suspension of 1-(4-iodophenyl)piperazine hydrochloride (300 mg, 0.924 mmol) and TBTU (297 mg, 0.924 mmol) in anhydrous DMF (5 mL) was added N,N-diisopropylethylamine (0.483 mL, 2.77 mmol) followed by (S)-(−)-2-methoxypropionic acid (96 mg, 0.924 mmol) at RT. The reaction was stirred at RT overnight, then concentrated under reduced pressure, deposited on Biotage® samplet and purified by flash chromatography (25 g SiO₂ Biotage® cartridge, using MeOH in CH₂Cl₂ eluting at 7% MeOH) to afford the desired product as a reddish gum (335 mg, 95% yield based on purity of 98%). LCMS: [M+H]⁺=375.26.

Step 2: (S)-1-(4-iodophenyl)-4-(2-methoxypropyl)piperazine

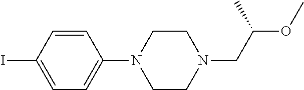 [SMILES: CO[C@@H](C)CN1CCN(c2ccc(I)cc2)CC1]

A dried vial was charged with (S)-1-(4-(4-iodophenyl)piperazin-1-yl)-2-methoxypropan-1-one (117 mg, 0.306 mmol) and filled with N₂. Anhydrous THF (10 mL) was added followed by (CH₃)₂S·BH₃ (2 M in THF, 0.46 mL, 0.92 mmol). After 5 min of stirring at RT the reaction was heated at 60° C. for 19.5 h. The reaction was then cooled to RT, carefully quenched with 2 M aq NaOH and concentrated under reduced pressure. Purification by flash chromatography (25 g SiO₂ Biotage® cartridge, using MeOH in CH₂Cl₂ eluting at 2% MeOH) afforded the product as a white solid (75.0 mg, 53% yield based on purity of 78%). LCMS: [M+H]⁺=361.19.

Step 3: (S)-3-chloro-6-fluoro-5-(4-(4-(2-methoxypropyl)piperazin-1-yl)phenyl)pyridin-2-amine

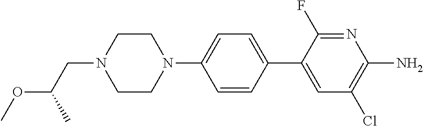 [SMILES: CO[C@@H](C)CN1CCN(c2ccc(-c3cc(Cl)c(N)nc3F)cc2)CC1]

A procedure similar to Example 1, General Method A using (S)-1-(4-iodophenyl)-4-(2-methoxypropyl)piperazine (0.075 g, 0.208 mmol), PdCl₂dppf (0.013 g, 0.017 mmol), Cs₂CO₃ (0.170 g, 0.523 mmol), H₂O (2 mL), DME (5 mL) and 3-chloro-6-fluoro-5-(4,4,5,5-tetramethyl-1,3,2-dioxaborolan-2-yl)pyridin-2-amine (1.571 mL, 0.209 mmol) in 1,4-dioxane (1.6 mL) afforded the crude product. Purification by flash chromatography (25 g SiO₂ Biotage® cartridge, using MeOH in CH₂Cl₂ eluting at 15% MeOH) afforded the product as a brown solid (109.0 mg, quant yield based on purity of 66%). LCMS: [M+H]⁺=379.32.

Step 4: (S)-6-(2-amino-6-fluoro-5-(4-(4-(2-methoxypropyl)piperazin-1-yl)phenyl)pyridin-3-yl)-3,4-dihydroisoquinolin-1(2H)-one

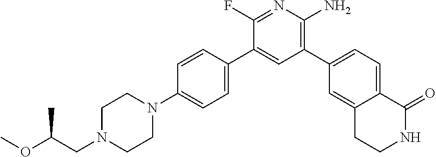 [SMILES: CO[C@@H](C)CN1CCN(c2ccc(-c3cc(-c4ccc5c(c4)CCNC5=O)c(N)nc3F)cc2)CC1]

A procedure similar to Example 1, General Method A using 6-(4,4,5,5-tetramethyl-1,3,2-dioxaborolan-2-yl)-3,4-dihydroisoquinolin-1(2H)-one (0.067 g, 0.247 mmol), K₃PO₄ (0.121 g, 0.570 mmol), XPhos Pd G2 (0.015 g, 0.019 mmol), H₂O (6 mL), (S)-3-chloro-6-fluoro-5-(4-(4-(2-methoxypropyl)piperazin-1-yl)phenyl)pyridin-2-amine (0.109 g, 0.190 mmol) and MeCN (9 mL) afforded the crude product. Purification by flash chromatography (25 g SiO₂ Biotage® cartridge, using MeOH in CH₂Cl₂ eluting at 9% MeOH) followed preparative HPLC (30 g Biotage® SNAP KP-C₁₈-HS, MeOH in (H₂O+0.05% TFA) eluting at 38% MeOH), afforded (S)-6-(2-amino-6-fluoro-5-(4-(4-(2-methoxypropyl)piperazin-1-yl)phenyl)pyridin-3-yl)-3,4-dihydroisoquinolin-1(2H)-one·TFA as an off white solid (32.0 mg, 27% yield based on purity of 95%). ¹H NMR (500 MHz, CD₃OD) b 8.05 (d, J=8.0 Hz, 1H), 7.63 (d, J=9.9 Hz, 1H), 7.50 (br d, J=8.7 Hz, 3H), 7.47 (s, 1H), 7.10 (brd, J=8.8 Hz, 2H), 3.77-3.99 (m, 3H), 3.62-3.75 (m, 2H), 3.51-3.61 (m, 2H), 3.45 (s, 3H), 3.21-3.30 (m, 4H), 3.03-3.11 (m, 2H), 1.26 (d, J=6.0 Hz, 3H). Signals corresponding to two aliphatic protons are obscured by the solvent/water peak. LCMS: [M +H]⁺=490.29.

### Example 25: (R)-6-(2-amino-6-fluoro-5-(4-(4-(2-methoxypropyl)piperazin-1-yl)phenyl)pyridin-3-yl)-3,4-dihydroisoquinolin-1(2H)-one (I-25)

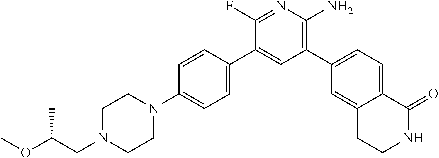 [SMILES: CO[C@H](C)CN1CCN(c2ccc(-c3cc(-c4ccc5c(c4)CCNC5=O)c(N)nc3F)cc2)CC1]

Step 1: (R)-1-(4-(4-iodophenyl)piperazin-1-yl)-2-methoxypropan-1-one

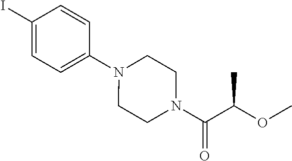 [SMILES: CO[C@H](C)C(=O)N1CCN(c2ccc(I)cc2)CC1]

To a suspension of 1-(4-iodophenyl)piperazine hydrochloride (300 mg, 0.924 mmol) and TBTU (297 mg, 0.924 mmol) in anhydrous DMF (5 mL) was added N,N-diisopropylethylamine (0.483 mL, 2.77 mmol) followed by (R)-2-methoxypropanoic acid (96 mg, 0.924 mmol) at RT. The reaction was stirred at RT overnight, then concentrated under reduced pressure, deposited on Biotage® samplet and purified by flash chromatography (25 g SiO₂ Biotage® cartridge, using MeOH in CH₂Cl₂) to afford the product as a reddish gum (392 mg, quant). LCMS: [M+H]⁺=375.03.

Step 2: (R)-1-(4-iodophenyl)-4-(2-methoxypropyl)piperazine

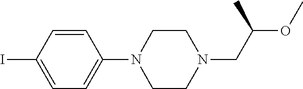 [SMILES: CO[C@H](C)CN1CCN(c2ccc(I)cc2)CC1]

A vial was charged with (R)-1-(4-(4-iodophenyl)piperazin-1-yl)-2-methoxypropan-1-one (118 mg, 0.309 mmol) and filled with N₂. Anhydrous THF (10 mL) was added, followed by (CH₃)₂S·BH₃ (2M in THF, 0.46 mL, 0.93 mmol). The reaction mixture was stirred at RT for 5 min and then heated at 60° C. overnight. After quenching with 2 M aq NaOH at RT, the mixture was concentrated under reduced pressure. Purification by flash chromatography (25 g SiO₂ Biotage® cartridge, using MeOH in CH₂Cl₂ eluting at 0-2% MeOH) afforded the product as a white solid (77.0 mg, 64% yield based on purity of 92%). LCMS: [M+H]⁺=361.04.

Step 3: (R)-3-chloro-6-fluoro-5-(4-(4-(2-methoxypropyl)piperazin-1-yl)phenyl)pyridin-2-amine

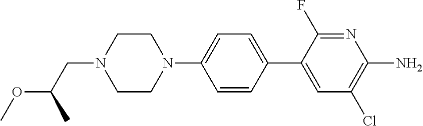 [SMILES: CO[C@H](C)CN1CCN(c2ccc(-c3cc(Cl)c(N)nc3F)cc2)CC1]

A procedure similar to Example 1, General Method A using (R)-1-(4-iodophenyl)-4-(2-methoxypropyl)piperazine (0.077 g, 0.197 mmol), PdCl₂dppf (0.013 g, 0.017 mmol), Cs₂CO₃ (0.170 g, 0.523 mmol), H₂O (2 mL), DME (2 mL) and crude 3-chloro-6-fluoro-5-(4,4,5,5-tetramethyl-1,3,2-dioxaborolan-2-yl)pyridin-2-amine (57 mg, 0.209 mmol) in 1,4-dioxane (1.74 mL) heating oil bath at 90° C. for 1.3 h afforded the crude product. Purification by flash chromatography (25 g SiO₂ Biotage® cartridge, using MeOH in CH₂Cl₂ eluting at 9% MeOH) afforded the product as an orange-tan solid (46.0 mg, 43% yield based on purity of 70%). LCMS: [M+H]⁺=379.32.

Step 4: Synthesis of (R)-6-(2-amino-6-fluoro-5-(4-(4-(2-methoxypropyl)piperazin-1-yl)phenyl)pyridin-3-yl)-3,4-dihydroisoquinolin-1(2H)-one

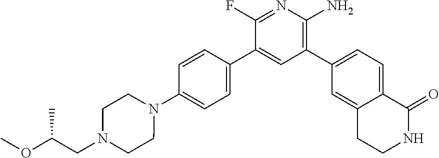 [SMILES: CO[C@H](C)CN1CCN(c2ccc(-c3cc(-c4ccc5c(c4)CCNC5=O)c(N)nc3F)cc2)CC1]

A procedure similar to Example 1, General Method A, using 6-(4,4,5,5-tetramethyl-1,3,2-dioxaborolan-2-yl)-3,4-dihydroisoquinolin-1(2H)-one (0.030 g, 0.110 mmol), K₃PO₄(0.054 g, 0.255 mmol), XPhos Pd G2 (6.69 mg, 8.50 μmol), H₂O (6 mL), (R)-3-chloro-6-fluoro-5-(4-(4-(2-methoxypropyl)piperazin-1-yl)phenyl)pyridin-2-amine (0.046 g, 0.085 mmol) and MeCN (9 mL) and heating in an oil bath at 100° C. for 3 h afforded the crude product. Purification by flash chromatography (25 g SiO₂ Biotage® cartridge, using MeOH in CH₂Cl₂ eluting at 9% MeOH) followed by preparative HPLC (30 g Biotage® SNAP KP-C₁₈-HS, MeOH in (H₂O+0.05% TFA) eluting at 40% MeOH) afforded the TFA salt of the title compound as a yellow-tan gum (37.0 mg, 70% yield based on purity of 97%). ¹H NMR (500 MHz, CD₃OD) δ 8.05 (d, J=8.0 Hz, 1H), 7.63 (d, J=9.8 Hz, 1H), 7.50 (br d, J=8.4 Hz, 3H), 7.47 (s, 1H), 7.10 (br d, J=8.7 Hz, 2H), 3.81-3.98 (m, 3H), 3.62-3.74 (m, 2H), 3.54-3.60 (m, 2H), 3.45 (s, 3H), 3.11-3.30 (m, 5H), 3.04-3.10 (m, 2H), 1.26 (d, J=6.0 Hz, 3H). A signal corresponding to an aliphatic proton is obscured by the solvent.

### Example 26: 6-(6′-amino-2′-fluoro-5-morpholino-[2,3′-bipyridin]-5′-yl)-3,4-dihydroisoquinolin-1(2H)-one (I-26)

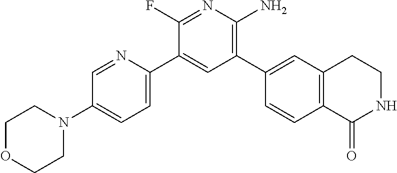 [SMILES: Nc1nc(F)c(-c2ccc(N3CCOCC3)cn2)cc1-c1ccc2c(c1)CCNC2=O]

Step 1: 5′-chloro-2′-fluoro-5-morpholino-[2, 3′-bipyridin]-6′-amine

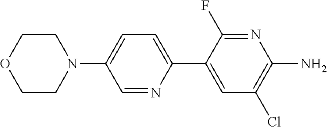 [SMILES: Nc1nc(F)c(-c2ccc(N3CCOCC3)cn2)cc1Cl]

A procedure similar to Example 1, General Method A using 4-(6-bromo pyridin-3-yl)morpholine (76 mg, 0.313 mmol), PdCl₂dppf (22.87 mg, 0.031 mmol), Cs₂CO₃ (305 mg, 0.938 mmol), H₂O (2 mL), DME (1.2 mL), 3-chloro-6-fluoro-5-(4,4,5,5-tetramethyl-1,3,2-dioxaborolan-2-yl)pyridin-2-amine (102 mg, 0.375 mmol) in 1,4-dioxane (2.8 mL) and heating in an oil bath at 90° C. for 1.5 h afforded the crude product. Purification by flash chromatography (25 g SiO₂ Biotage® cartridge, using MeOH in CH₂Cl₂ eluting at 6% MeOH) afforded the product as a light orange solid (53 mg, 50% yield based on purity of 91%). LCMS: [M+H]⁺=309.29.

Step 2: 6-(6′-amino-2′-fluoro-5-morpholino-[2,3′-bipyridin]-5′-yl)-3,4-dihydroisoquinolin-1(2H)-one

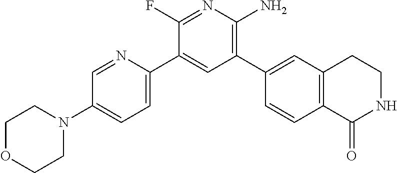 [SMILES: Nc1nc(F)c(-c2ccc(N3CCOCC3)cn2)cc1-c1ccc2c(c1)CCNC2=O]

A procedure similar to General Method A, Example 1 using 5′-chloro-2′-fluoro-5-morpholino-[2,3′-bipyridin]-6′-amine (53 mg, 0.156 mmol), K₃PO₄ (99 mg, 0.469 mmol), 6-(4,4,5,5-tetramethyl-1,3,2-dioxaborolan-2-yl)-3,4-dihydroisoquinolin-1(2H)-one (55.5 mg, 0.203 mmol), H₂O (6 mL), MeCN (9 mL), XPhos Pd G2 (12.29 mg, 0.016 mmol); by heating in an oil bath at 100° C. for 3.4 h. Purification by flash chromatography (25 g SiO₂ Biotage® cartridge, using MeOH in CH₂Cl₂ eluting at 5% MeOH) followed by preparative HPLC (30 g Biotage® SNAP KP-C₁₈-HS, MeOH in H₂O eluting at 66% MeOH) and a filtration through a Waters PoraPak CX afforded the title compound as a pale yellow solid (9 mg, 13% yield based on purity of 95%). ¹H NMR (500 MHz, CD3OD) δ 8.28 (br s, 1H), 8.06 (d, J=7.8 Hz, 1H), 8.00 (br d, J=9.4 Hz, 1H), 7.75 (br s, 1H), 7.56-7.63 (m, 1H), 7.54 (br d, J=7.7 Hz, 1H), 7.49 (s, 1H), 3.86-3.91 (m, 4H), 3.55-3.60 (m, 2H), 3.26-3.30 (m, 4H), 3.04-3.11 (m, 2H); LCMS: [M+H]⁺=420.39.

### Example 27: 6-(6′-amino-2′-fluoro-5-(4methylpiperazin-1-yl)-[2,3′-bipyridin]-5′-yl)-3,4dihydroisoquinolin-1(2H)-one acetate (1-27)

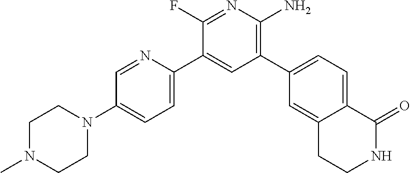 [SMILES: CN1CCN(c2ccc(-c3cc(-c4ccc5c(c4)CCNC5=O)c(N)nc3F)nc2)CC1]

Step 1: 5′-chloro-2′-fluoro-5-(4-methylpiperazin-1-yl)-[2, 3′-bipyridin]-6′-amine

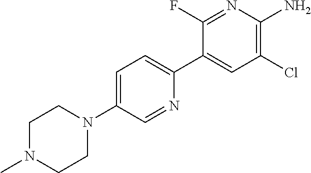 [SMILES: CN1CCN(c2ccc(-c3cc(Cl)c(N)nc3F)nc2)CC1]

A procedure similar to that described in Example 1, General Method A using 1-(6-bromopyridin-3-yl)-4-methylpiperazine (0.080 g, 0.312 mmol), PdCl₂dppf (0.023 g, 0.031 mmol), Cs₂CO₃ (0.305 g, 0.938 mmol), H₂O (2 mL), DME (1.2 mL) and 3-chloro-6-fluoro-5-(4,4,5,5-tetramethyl-1,3,2-dioxaborolan-2-yl)pyridin-2-amine (2.82 mL, 36.25 mg/mL, 0.375 mmol) in 1,4-dioxane (2.8 mL) and heating in a microwave reactor at 90° C. for 1 h provided after purification of the crude product by flash chromatography (25 g SiO₂, using MeOH in CH₂Cl₂) to give the product as a light orange solid (70 mg, 70% yield). LCMS: [M+H]⁺=322.23.

Step 2: Step 3: 6-(6′-amino-2′-fluoro-5-(4-methylpiperazin-1-yl)-[2,3′-bipyridin]-5′-yl)-3,4-dihydroisoquinolin-1(2H)-one, acetic acid

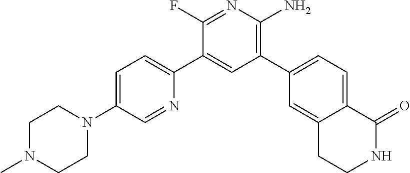 [SMILES: CN1CCN(c2ccc(-c3cc(-c4ccc5c(c4)CCNC5=O)c(N)nc3F)nc2)CC1]

Following a procedure similar to Example 1, General Method A, a degassed and sealed mixture of 5′-chloro-2′-fluoro-5-(4-methylpiperazin-1-yl)-[2,3′-bipyridin]-6′-amine (70 mg, 0.218 mmol), 3,4-dihydro-1(2H)-isoquinolinone-6-boronic acid pinacol ester (77 mg, 0.283 mmol), K₃PO₄ (139 mg, 0.653 mmol), H₂O (6 mL), MeCN (9 mL) and XPhos Pd G2 (171 mg, 0.218 mmol) was heated in an oil bath at 100° C. for 4 h. The crude product was purified by flash chromatography (25 g SiO₂, using MeOH in CH₂Cl₂) to afford the title compound as an off white solid (35.0 mg, 33% yield based on purity of 99.7%). ¹H NMR (500 MHz, DMSO-d₆) δ 8.35 (d, J=2.7 Hz, 1H), 8.09 (d, J=10.0 Hz, 1H), 7.87-8.00 (m, 2H), 7.60 (br d, J=8.1 Hz, 1H), 7.35-7.46 (m, 3H), 6.36 (s, 2H), 3.39-3.43 (m, 2H), 3.22 (br d, J=4.5 Hz, 4H), 2.94-3.01 (m, 2H), 2.44-2.48 (m, 4H), 2.24 (s, 3H), 1.91 (s, 3H); LCMS: [M+H]⁺=433.33.

### Example 28: 6-(2-amino-6-fluoro-5-(4-(hydroxydimethylsilyl)phenyl)pyridin-3-yl)-3,4-dihydroisoquinolin-1(2H)-one (I-28

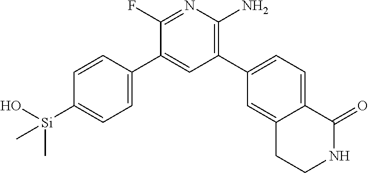 [SMILES: C[Si](C)(O)c1ccc(-c2cc(-c3ccc4c(c3)CCNC4=O)c(N)nc2F)cc1]

Step 1: dimethyl(4-(4,4,5,5-tetramethyl-1,3,2-dioxaborolan-2-yl)phenyl)silanol

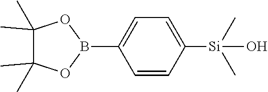 [SMILES: CC1(C)OB(c2ccc([Si](C)(C)O)cc2)OC1(C)C]

A microwave vial was charged with dimethyl(4-chlorophenyl)silanol (250 mg, 1.339 mmol), B₂pin₂ (340 mg, 1.339 mmol), KOAc (394 mg, 4.02 mmol), XPhos Pd G2 (105 mg, 0.134 mmol). 1,4-Dioxane (10 mL) was added and the reaction was degassed with N₂. The sealed vial was than heated in an oil bath at 90° C. for 18 h. The resulting product used as the crude solution (37.3 mg/mL) in the subsequent Suzuki-Miyaura cross coupling reaction.

Step 2: (4-(6-amino-5-chloro-2-fluoropyridin-3-yl)phenyl)dimethylsilanol

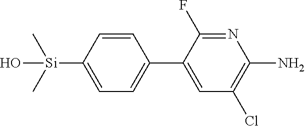 [SMILES: C[Si](C)(O)c1ccc(-c2cc(Cl)c(N)nc2F)cc1]

A procedure similar to Example 1, General Method A using of 5-bromo-3-chloro-6-fluoropyridin-2-amine (80 mg, 0.355 mmol), Cs₂CO₃ (347 mg, 1.065 mmol), PdCl₂dppf (26.0 mg, 0.035 mmol), H₂O (1.6 mL), dimethyl(4-(4,4,5,5-tetramethyl-1,3,2-dioxaborolan-2-yl)phenyl)silanol (118 mg, 37.3 mg/mL, 0.426 mmol) in 1,4-dioxane (3.2 mL) and heating at 90° C. for 1 h afforded, after purification by flash chromatography (25 g SiO₂, using MeOH in CH₂Cl₂) the product as a tan solid (60.0 mg, 41% yield based on purity of 72%). LCMS: [M+H]⁺=297.26.

Step 3: 6-(2-amino-6-fluoro-5-(4-(hydroxydimethylsilyl)phenyl)pyridin-3-yl)-3,4-dihydroisoquinolin-1(2H)-one

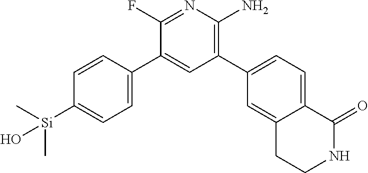 [SMILES: C[Si](C)(O)c1ccc(-c2cc(-c3ccc4c(c3)CCNC4=O)c(N)nc2F)cc1]

A procedure similar to Example 1, General Method A using 3,4-dihydro-1(2H)-isoquinolinone-6-boronic acid pinacol ester (71.8 mg, 0.263 mmol), K₃PO₄ (129 mg, 0.606 mmol), (4-(6-amino-5-chloro-2-fluoropyridin-3-yl)phenyl)dimethylsilanol (60 mg, 0.145 mmol, 72%) in H₂O (6 mL), MeCN (12 mL) and XPhos Pd G2 (15.91 mg, 0.020 mmol) and heating in a microwave reactor at 100° C. for 2 h afforded, after purification by flash chromatography (25 g SiO₂, using MeOH in CH₂Cl₂) followed by preparative HPLC (30 g Biotage® SNAP KP-C₁₈-HS, MeOH in H₂O eluting at 60% MeOH), the title compound as an off white solid (13.0 mg, 21% yield based on purity of 97%). ¹H NMR (500 MHz, CD₃OD) δ 8.05 (d, J=7.8 Hz, 1H), 7.61-7.71 (m, 3H), 7.49-7.59 (m, 3H), 7.46 (s, 1H), 3.56 (br t, J=6.6 Hz, 2H), 3.07 (br t, J=6.5 Hz, 2H), 0.35-0.41 (m, 6H); LCMS: [M+H]⁺=408.28.

### Example 29: 6-(2-amino-6-fluoro-5-(4-(4-(2-methoxyethyl)piperazin-1-yl)phenyl)pyridin-3-yl)-3,4-dihydroisoquinolin-1(2H)-one (I-29)

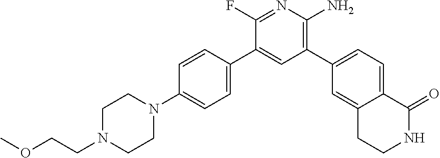 [SMILES: COCCN1CCN(c2ccc(-c3cc(-c4ccc5c(c4)CCNC5=O)c(N)nc3F)cc2)CC1]

Step 1: 3-chloro-6-fluoro-5-(4,4,5,5-tetramethyl-1,3,2-dioxaborolan-2-yl)pyridin-2-amine

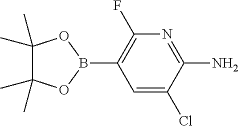 [SMILES: CC1(C)OB(c2cc(Cl)c(N)nc2F)OC1(C)C]

To a vial containing 5-bromo-3-chloro-6-fluoropyridin-2-amine (425 mg, 1.89 mmol), bis(pinacolato)diboron (479 mg, 1.89 mmol), tris(dibenzylideneacetone)dipalladium (0) (173 mg, 0.189 mmol), tricyclohexylphosphine (52.9 mg, 0.189 mmol) and KOAc (370 mg, 3.77 mmol), was added 1,4-dioxane (15 mL). The reaction mixture was degassed with argon, the vial was sealed and then heated at 90° C. for 16 h. The reaction mixture was filtered through a Celite® bed, which was washed in with EtOAc (50 mL). The combined filtrate was concentrated under reduced pressure, prepared a standard stock solution of known concentration in DME and carried forward to subsequent step without further purification. LCMS: [M+H]⁺=273.34.

Step 2: 1-(4-iodophenyl)-4-(2-methoxyethyl)piperazine

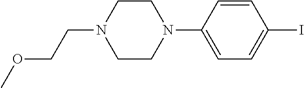 [SMILES: COCCN1CCN(c2ccc(I)cc2)CC1]

A microwave vial containing a slurry of 1,4-diiodobenzene (500 mg, 1.516 mmol), 1-(2-methoxyethyl)piperazine (219 mg, 1.52 mmol), 4,5-bis(diphenylphosphino)-9,9-dimethylxanthene (79 mg, 0.136 mmol), tris(dibenzylideneacetone)dipalladium (0) (41.6 mg, 0.045 mmol) and potassium tert-butoxide (510 mg, 4.55 mmol) in toluene (10 ml) was flushed with argon, was heated in an oil bath at 100° C. for 3 h. The reaction was partitioned between EtOAc (15 mL) and water (20 mL). The layers were separated and the aqueous layer was extracted with additional EtOAc (x2). The combined organic extracts were dried and concentrated onto Celite®. Silica gel chromatography (eluting with CH₂Cl₂ containing 0-2% methanol and 0-0.2% ammonium hydroxide) afforded the product as a pale yellow solid (205 mg, 39%). LCMS: [M+H]⁺=347.25

Step 3: 3-chloro-6-fluoro-5-(4-(4-(2-methoxyethyl)piperazin-1-yl)phenyl)pyridin-2-amine

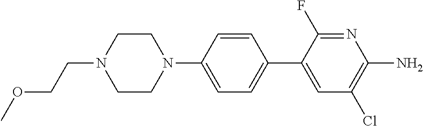 [SMILES: COCCN1CCN(c2ccc(-c3cc(Cl)c(N)nc3F)cc2)CC1]

A microwave vial charged with [1,1′-bis(diphenylphosphino)ferrocene]dichloropalladium(II) (12.68 mg, 0.017 mmol), 3-chloro-6-fluoro-5-(4,4,5,5-tetramethyl-1,3,2-dioxaborolan-2-yl)pyridin-2-amine (55 mg, 0.202 mmol), 1-(4-iodophenyl)-4-(2-methoxyethyl)piperazine (50 mg, 0.144 mmol), DME (3.8 mL) and water (1 mL) was flushed with argon The vial was sealed and the reaction mixture was heated at 85° C. in a microwave reactor for 1.75 h. The reaction was partitioned between EtOAc and brine. The layers were separated and the aqueous layer was extracted with additional EtOAc (x2). The combined organic extracts were dried and concentrated onto Celite®. Silica gel chromatography (eluting with CH₂Cl₂ containing 0-2% methanol and 0-0.2% NH₄OH) followed by filtration through a Waters PoraPak CX column afforded the product as a beige solid (47 mg, 79% yield based on purity of 89%). LCMS: [M+H]⁺=365.37.

Step 4: 6-(2-amino-6-fluoro-5-(4-(4-(2-methoxyethyl)piperazin-1-yl)phenyl)pyridin-3-yl)-3,4-dihydroisoquinolin-1(2H)-one

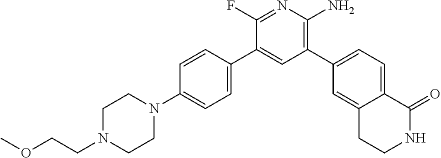 [SMILES: COCCN1CCN(c2ccc(-c3cc(-c4ccc5c(c4)CCNC5=O)c(N)nc3F)cc2)CC1]

A microwave vial charged with XPhos Pd G2 (11.67 mg, 0.015 mmol), 3,4-dihydro-1(2H)-isoquinolinone-6-boronic acid pinacol ester (40.5 mg, 0.148 mmol, 3-chloro-6-fluoro-5-(4-(4-(2-methoxyethyl)piperazin-1-yl)phenyl)pyridin-2-amine (47 mg, 0.114 mmol), Cs₂CO₃ (112 mg, 0.342 mmol), 1,2-dimethoxyethane (5 ml) and water (1 ml) was flushed with argon. The vial was sealed and the reaction mixture was heated at 90° C. in a microwave reactor for 1 h. The reaction was partitioned between EtOAc and brine. The layers were separated and the aqueous layer was extracted with additional EtOAc (x2). The combined organic extracts were dried and concentrated onto Celite®. Silica gel chromatography (eluting with CH₂Cl₂ containing 0-2% methanol and 0-0.2% NH₄OH) followed by reverse phase chromatography (C18; 0-30% acetonitrile/water) afforded the title compound as a white solid (11 mg, 19% yield based on purity of 95%). ¹H NMR (500 MHz, DMSO-d₆) δ 7.9-8.0 (m, 2H), 7.6-7.6 (m, 1H), 7.4-7.5 (m, 2H), 7.4-7.4 (m, 2H), 6.9-7.1 (m, 2H), 6.1-6.3 (m, 2H), 3.5-3.5 (m, 2H), 3.4-3.4 (m, 4H), 3.2-3.3 (m, 3H), 3.1-3.2 (m, 4H), 2.9-3.0 (m, 2H), 2.6-2.6 (m, 4H). LCMS: [M+H]⁺=476.56.

### Example 30: 6-(2-amino-6-fluoro-5-(4-(piperidin-4-yl)phenyl)pyridin-3-yl)-3,4-dihydroisoquinolin-1(2H)-one (I-30)

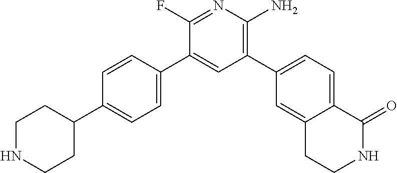 [SMILES: Nc1nc(F)c(-c2ccc(C3CCNCC3)cc2)cc1-c1ccc2c(c1)CCNC2=O]

Step 1: 3-chloro-6-fluoro-5-(4-(piperidin-4-yl) phenyl) pyridin-2-amine

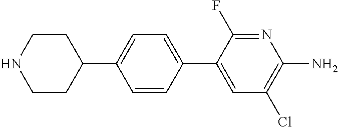 [SMILES: Nc1nc(F)c(-c2ccc(C3CCNCC3)cc2)cc1Cl]

A procedure analogous to that of Example 29, Step 3 was used, by coupling 4-(4-bromophenyl)piperidine (150 mg, 0.625 mmol) and 3-chloro-6-fluoro-5-(4,4,5,5-tetramethyl-1,3,2-dioxaborolan-2-yl)pyridin-2-amine (221 mg, 0.812 mmol). After the standard workup and silica gel chromatography (eluting with CH₂Cl₂ containing 0-3.5% methanol and 0-0.35% NH₄OH) followed by filtration through a Waters PoraPak CX column afforded the product as a beige solid (133 mg, 64% yield based on purity of 92%). LCMS: [M +H]⁺=306.38

Step 2: 6-(2-amino-6-fluoro-5-(4-(piperidin-4-yl)phenyl)pyridin-3-yl)-3,4-dihydroisoquinolin-1(2H)-one

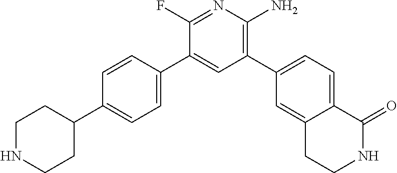 [SMILES: Nc1nc(F)c(-c2ccc(C3CCNCC3)cc2)cc1-c1ccc2c(c1)CCNC2=O]

A procedure analogous to that of Example 29, Step 4 was used, coupling 3-chloro-6-fluoro-5-(4-(piperidin-4-yl)phenyl)pyridin-2-amine (133 mg, 0.400 mmol) and 3,4-dihydro-1(2H)-isoquinolinone-6-boronic acid pinacol ester (142 mg, 0.520 mmol). The reaction was partitioned between EtOAc and brine. The layers were separated and the aqueous layer was extracted with CHCl₃/IPA 4:1 mix (2×3 mL). The combined organic extracts were dried and concentrated onto Celite®. Reverse phase chromatography (C18; 0-35% acetonitrile/water) followed by filtration through a Waters PoraPak CX column afforded the title compound as an off white solid (66 mg, 40% yield based on purity of 95%). ¹H NMR (500 MHz, DMSO-d₆) δ 7.9-8.0 (m, 2H), 7.6-7.7 (m, 1H), 7.4-7.5 (m, 4H), 7.2-7.4 (m, 2H), 6.2-6.4 (m, 2H), 4.0-4.2 (m, 1H), 3.4-3.4 (m, 2H), 3.0-3.1 (m, 2H), 2.9-3.0 (m, 2H), 2.6-2.7 (m, 3H), 1.7-1.8 (m, 2H), 1.4-1.6 (m, 2H). LCMS: [M+H]⁺=417.52

### Example 31: 6-(2-amino-6-fluoro-5-(4-(1-methyl-5,6-dihydro-1,2,4-triazin-4(1H)-yl)phenyl)pyridin-3-yl)-3,4-dihydroisoquinolin-1(2H)-one (I-31)

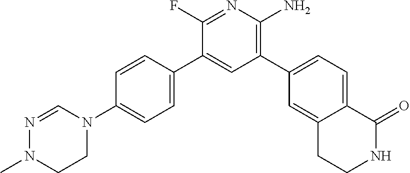 [SMILES: CN1CCN(c2ccc(-c3cc(-c4ccc5c(c4)CCNC5=O)c(N)nc3F)cc2)C=N1]

Step 1: 5-bromo-6-fluoropyridin-2-amine:

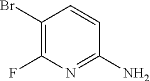

To a solution of 6-fluoropyridin-2-amine (3 g, 26.8 mmol) in CH₃CN (100 mL) under argon atmosphere was added NBS (4.7 g, 26.8 mmol) in CH₃CN (80 mL) dropwise for 30 min at 0° C. and stirred for another 30 min at RT. The solvent was evaporated under reduced pressure and the resulting crude product was purified by column chromatography (silica gel, 100-200 mesh) using 10% EtOAc in pet ether as an eluent to afford the product (4.4 g, 88%) as a yellow solid. LCMS: [M+H]⁺=191.12.

Step 2: 5-bromo-3-chloro-6-fluoropyridin-2-amine:

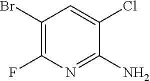

To a solution of 5-bromo-6-fluoropyridin-2-amine (2 g, 10.5 mmol) in CH₃CN (30 mL) was added NCS (1.4 g, 10.5 mmol) portionwise and the suspension was heated at reflux for 2 h at 90° C. under argon atmosphere. The reaction mixture was allowed to cool to RT, and was poured into ice-cold water (100 mL) to give a precipitate which was filtered to isolate the crude compound. Washing with n-pentane (100 mL) afforded the product (1.1 g, 48%) as an off-white solid. LCMS: [M+H]⁺=225.09.

Step 3: 3-chloro-6-fluoro-5-(4,4,5,5-tetramethyl-1,3,2-dioxaborolan-2-yl)pyridin-2-amine

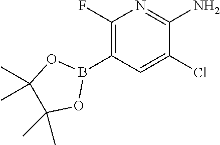 [SMILES: CC1(C)OB(c2cc(Cl)c(N)nc2F)OC1(C)C]

To a vial charged with B₂pin₂ (367 mg, 1.45 mmol), Pd₂dba₃ (132 mg, 0.145 mmol), PCy₃ (40.6 mg, 0.145 mmol), 5-bromo-3-chloro-6-fluoropyridin-2-amine (326 mg, 1.446 mmol) and KOAc (284 mg, 2.89 mmol) was added anhydrous 1,4-dioxane (12 mL) was added. The suspension was degassed by evacuation and refilled with N₂. The reaction mixture was then heated while sealed in an oil bath at 90-95° C. for 19 h. The resulting crude solution of product (36.2 mg/mL) was used in the subsequent step assuming quantitative conversion. LCMS: [M+H]⁺=273.27.

Step 4: 2-((4-bromophenyl)amino)ethan-1-ol:

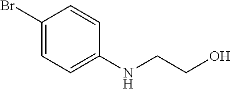 [SMILES: OCCNc1ccc(Br)cc1]

To a solution of 1-bromo-4-iodobenzene (10 g, 35.5 mmol) in DMSO (100 mL) was added KOH (3.98 g, 70.9 mmol) and CuCl (0.7 g, 7.07 mmol) followed by ethanol amine (6.5 g, 106.4 mmol) at RT. The mixture was stirred at RT for 24 h, then quenched with ice water (100 mL) and extracted with EtOAc (2×100 mL). The combined organic layer was dried over Na₂SO₄ and concentrated under reduced pressure. The resulting residue was purified by column chromatography (using 100-200 silica gel) eluting with 30-40% EtOAc in pet ether to afford the product (5 g, 66%) as a pale yellow solid. LCMS: [M+H]⁺=216.23.

Step 5: 2-((4-bromophenyl)amino)ethyl methanesulfonate:

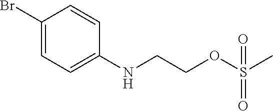

To a solution of 2-((4-bromophenyl)amino)ethan-1-ol (5 g, 23.2 mmol) in DCM (50 mL) was added triethylamine (9.8 mL, 69.7 mmol), followed by mesyl chloride (4.5 g, 58.2 mmol) at 0° C. to RT for 2 h. Then, the reaction mixture was poured into ice water (100 mL) and extracted with DCM (2×100 mL). The combined organic layers were dried over Na₂SO₄ and concentrated under reduced pressure to afford the crude product (5 g, quant) as a pale yellow solid. The crude compound was used without further purification. LCMS: [M+2H]⁺=295.99.

Step 6: 4-bromo-N-(2-(1-methylhydrazinyl)ethyl)aniline:

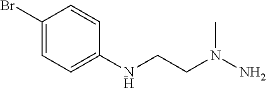

To a solution of 2-((4-bromophenyl)amino)ethyl methanesulfonate (5 g, 17.1 mmol) in ethanol (50 mL) was added DIPEA (14.3 mL, 85.3 mmol) followed by 85% aq. methyl hydrazine (10 mL, 2V) at RT. The mixture was heated to 80-85° C. for 2 h before cooling to RT. The solvent was concentrated under reduced pressure to give the crude compound which was poured into ice water (100 mL) and extracted with DCM (2×100 mL). The combined organic layers were dried over Na₂SO₄ and concentrated under reduced pressure to afford the crude product (3 g, 31%) as a pale brown solid. The crude compound was used without further purification. LCMS: [M+H]⁺: 244.22.

Step 7: 4-(4-bromophenyl)-1-methyl-1,4,5,6-tetrahydro-1,2,4-triazine:

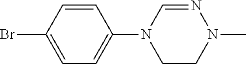

To a solution of 4-bromo-N-(2-(1-methylhydrazinyl)ethyl)aniline (30.52% pure by LCMS) (5 g (1.5 g), 6.2 mmol) in AcOH (20 mL, 15V) was added trimethylorthoformate (20 mL, 15V) at 80° C. to 85° C. for 1 h before cooling to RT. The crude compound was poured into ice water (100 mL) basified with saturated NaHCO₃ solution and extracted with DCM (2×50 mL). The combined organic layer was dried over Na₂SO₄ and concentrated under reduced pressure to give crude residue: which was purified by column chromatography (silica gel 100-200 mesh) using an eluent with 70-90% EtOAc in petroleum ether to afford the product (620 mg, 40%) as a pale yellow solid. LCMS: [M+H]⁺=254.25.

Step 8: 3-chloro-6-fluoro-5-(4-(1-methyl-5,6-dihydro-1,2,4-triazin-4(1H)-yl)phenyl)pyridin-2-amine

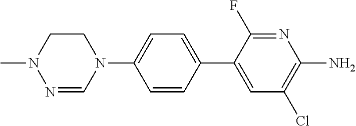 [SMILES: CN1CCN(c2ccc(-c3cc(Cl)c(N)nc3F)cc2)C=N1]

A method similar to General Method A using Pd(amphos)Cl₂ (27.9 mg, 0.039 mmol), 4-(4-bromophenyl)-1-methyl-1,4,5,6-tetrahydro-1,2,4-triazine (100 mg, 0.393 mmol), K₃PO₄ (251 mg, 1.180 mmol), H₂O (1 mL), 1,4-dioxane (7 mL) and 3-chloro-6-fluoro-5-(4,4,5,5-tetramethyl-1,3,2-dioxaborolan-2-yl)pyridin-2-amine (107 mg, 0.393 mmol, 36.2 mg/mL) in 1,4-dioxane solution (2.96 mL) and heating the degassed and sealed mixture under microwave irradiation at 90° C. for 1.5 h gave, after purification by flash chromatography (25 g SiO₂, using MeOH in CH₂Cl₂) the product as a brown solid (44% purity) that was used as is in the next step. LCMS: [M+H]⁺=320.12.

Step 9: 6-(2-amino-6-fluoro-5-(4-(1-methyl-5,6-dihydro-1,2,4-triazin-4(1H)-yl)phenyl)pyridin-3-yl)-3,4-dihydroisoquinolin-1(2H)-one

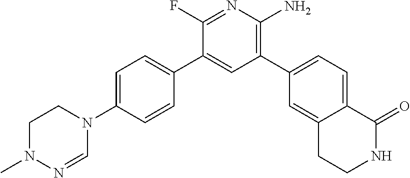 [SMILES: CN1CCN(c2ccc(-c3cc(-c4ccc5c(c4)CCNC5=O)c(N)nc3F)cc2)C=N1]

The procedure followed was similar to Example 1, General Method A using 3-chloro-6-fluoro-5-(4-(1-methyl-5,6-dihydro-1,2,4-triazin-4(1 H)-yl)phenyl)pyridin-2-amine (entire material from the previous step), K₃PO₄ (251 mg, 1.182 mmol), 3,4-dihydro-1(2H)-isoquinolinone-6-boronic acid pinacol ester (140 mg, 0.512 mmol), H₂O (6 mL), MeCN (9 mL) and XPhos Pd G2 (31.0 mg, 0.039 mmol) and heating the sealed, degassed mixture in a microwave reactor at 100° C. for 1 h. The reaction mixture was then concentrated and purified by flash chromatography (25 g SiO₂, using MeOH in CH₂Cl₂) followed, in sequence, by preparative HPLC (30 g Biotage® SNAP KP-C₁₈-HS, MeOH in H₂O), a filtration through a Waters PoraPak CX column and then another flash chromatography (50 g SiO₂, using MeOH in CH₂Cl₂) to afford the title compound as a white solid (16.0 mg, 9% yield for 2 steps, based on purity of 99%). ¹H NMR (500 MHz, DMSO-d₆) δ ppm 7.94 (br s, 1H), 7.91 (d, J=8.0 Hz, 1H), 7.63 (d, J=10.2 Hz, 1H), 7.48-7.53 (m, 3H), 7.45-7.48 (m, 1H), 7.42 (s, 1H), 7.18 (br d, J=8.7 Hz, 2H), 6.25 (s, 2H), 3.75 (br t, J=4.8 Hz, 2H), 3.37-3.45 (m, 2H), 2.90-2.99 (m, 4H), 2.65 (s, 3H); LCMS: [M+H]⁺=431.15.

### Example 32: 5-(2-amino-6-fluoro-5-(4-(4-methylpiperazin-1-yl)phenyl)pyridin-3-yl)isoindolin-1-one (I-32)

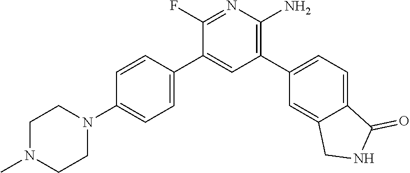 [SMILES: CN1CCN(c2ccc(-c3cc(-c4ccc5c(c4)CNC5=O)c(N)nc3F)cc2)CC1]

Step 1: 3-chloro-6-fluoro-5-(4-(4-methylpiperazin-1-yl)phenyl)pyridin-2-amine

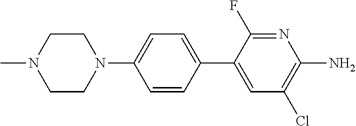 [SMILES: CN1CCN(c2ccc(-c3cc(Cl)c(N)nc3F)cc2)CC1]

In a microwave vial with magnetic stir bar was placed 5-bromo-3-chloro-6-fluoropyridin-2-amine (260 mg, 1.15 mmol, preparation described in Example 31, Step 2), 4-(4-methylpiperazin-1-yl)phenylboronic acid, pinacol ester (383 mg, 1.27 mmol), [1,1′-bis(diphenylphosphino)ferrocene]dichloropalladium(II) (84 mg, 0.115 mmol), Cs₂CO₃ (1127 mg, 3.46 mmol), DME (4 mL) and H₂O (2 mL) (4:1 DME/H₂O). The flask was sealed, then heated to 90° C. for 1 h in the microwave (high absorbance) when LCMS indicated complete conversion [01]. The reaction was loaded onto Celite® and purified by reverse phase chromatography (C₁₈, MeCN—H₂O) to give the product as a white solid (204 mg, 55%). ¹H NMR (500 MHz, DMSO-d₆) δ 7.80 (d, J=8.9 Hz, 1H), 7.35 (d, J=7.6 Hz, 2H), 6.96 (d, J=8.9 Hz, 2H), 6.66 (s, 2H), 3.18-3.14 (m, 4H), 2.46-2.42 (m, 4H), 2.22 (s, 3H); LCMS: [M+H]⁺=323.28.

Step 2: 5-(2-amino-6-fluoro-5-(4-(4-methylpiperazin-1-yl)phenyl)pyridin-3-yl)isoindolin-1-one

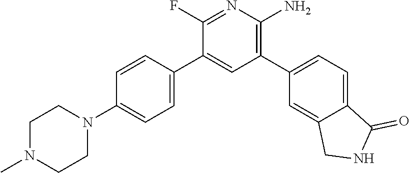 [SMILES: CN1CCN(c2ccc(-c3cc(-c4ccc5c(c4)CNC5=O)c(N)nc3F)cc2)CC1]

In a microwave vial with magnetic stir bar was placed 3-bromo-6-fluoro-5-(4-(4-methylpiperazin-1-yl)phenyl)pyridin-2-amine (55 mg, 0.151 mmol), 5-(4,4,5,5-tetramethyl-1,3,2-dioxaborolan-2-yl)isoindolin-1-one (58.5 mg, 0.226 mmol), XPhos Pd G2 (5.92 mg, 7.53 μmol), Cs₂CO₃ (147 mg, 0.452 mmol), DME (4 mL) and H₂O (2 mL) (4:1 DME/H₂O). The flask was sealed, then heated to 90° C. for 1 h in a microwave (high absorbance), the reaction was then loaded onto Celite® and purified by reverse phase chromatography (C18, MeCN—H₂O) to give the provide the title compound (12 mg, 18%) as a white solid. ¹H NMR (500 MHz, DMSO-d₆) δ 8.61 (s, 1H), 7.78 (d, J=7.8 Hz, 1H), 7.74 (s, 1H), 7.67-7.61 (m, 2H), 7.45 (br d, J=8.1 Hz, 2H), 7.02 (br d, J=8.7 Hz, 2H), 6.22 (s, 2H), 4.46 (s, 2H), 3.24-3.18 (m, 4H), 2.55 (br s, 4H), 2.27 (s, 3H); LCMS: [M+H]⁺=418.29.

### Example 33: 5-(2-amino-6-fluoro-5-(4-(4-Methylpiperazin-1-yl)phenyl)pyridin-3-yl)-3,3-dimethylisoindolin-1-one (I-33)

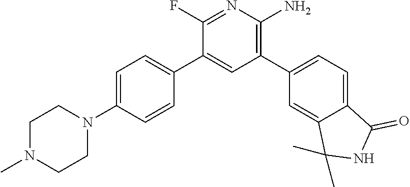 [SMILES: CN1CCN(c2ccc(-c3cc(-c4ccc5c(c4)C(C)(C)NC5=O)c(N)nc3F)cc2)CC1]

Step 1. methyl 4-bromo-2-(bromomethyl)benzoate (2):

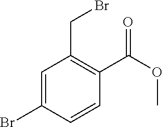

To a stirred solution of methyl 4-bromo-2-methylbenzoate (10 g, 43.9 mmol) in CCl₄ (100 mL), NBS (8.6 g, 48.3 mmol) and AIBN (0.730 g, 4.5 mmol) were added and the suspension was heated at reflux for 4 h at 80° C. under argon atmosphere before cooling to RT. The reaction mixture was filtered through the Celite® bed which was washed with diethyl ether (250 mL). The filtrate was concentrated under reduced pressure and dissolved in DCM (250 mL) and washed with cold water (3×50 mL). The combined organic layer was dried over anhydrous Na₂SO₄ and concentrated under reduced pressure to afford the product (9.6 g, 71%) as a yellow oil. TLC: 20% EtOAc: Pet ether; Rf: 0.6

Step 2: 5-bromoisoindolin-1-one (3):

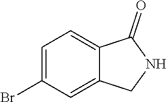 [SMILES: O=C1NCc2cc(Br)ccc21]

A solution of NH₃ in methanol (100 mL, 7M) was added to the crude methyl 4-bromo-2-(bromomethyl)benzoate (6 g, 19.5 mmol) and heated to 90° C. for 16 h in a steel bomb before cooling to RT. The solid precipitate which was formed was filtered and washed with methanol (25 mL). The residue was dried to afford compound 3 (3.2 g, 78%) as a light brown solid. which was enough pure to be used in next step. LCMS: [M+2H]⁺=212.14.

Step 3: 5-bromo-2-(4-methoxybenzyl)-3,3-dimethylisoindolin-1-one (4):

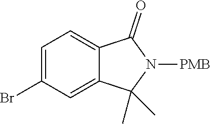

To a stirred solution of tetra-n-butyl ammonium iodide (2.30 g, 6.2 mmol) in THF (190 mL), NaH (1.24 g, 51.6 mmol) was added at RT under argon atmosphere, followed by a solution 5-bromoisoindolin-1-one (6.4 g, 30.3 mmol) in DMF:THF (50:190 mL). After 75 min, 4-methoxybenzylbromide (4.6 mL, 31.9 mmol) was added and stirring was continued for 4 h. Then NaH (6.03 g, 251.3 mmol) was added and stirring was continued for 30 min before iodomethane (11.5 mL, 184.7 mmol) was added. The mixture was heated to 70° C. for 1 h before cooling to RT. The reaction mixture was then quenched with saturated aqueous NH4Cl solution (75 mL) and extracted with EtOAc (3×150 mL) and saturated brine solution (3×50 mL). The combined organic layers were dried over anhydrous Na₂SO₄, filtered and concentrated under reduced pressure to afford crude product which was purified using a silica flash cartridge (40 g column) using 8-15% EtOAc in pet ether as an eluent to afford the product (2.84 g, 27%) as yellow oil. LCMS: [M+2H]⁺=360.29.

Step 4. 5-bromo-3,3-dimethylisoindolin-1-one (5):

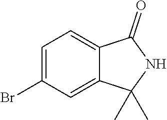

Ceric ammonium nitrate (13.13 g, 24.0 mmol) was added to a stirred solution of 5-bromo-2-(4-methoxybenzyl)-3,3-dimethylisoindolin-1-4 (2.84 g, 7.9 mmol) in acetonitrile (52 mL) and water (23 mL) at 0° C. and stirred for 1 h. The reaction mixture was extracted with EtOAc (3×100 mL) and saturated brine solution (3×50 mL). Combined organic layer was dried over anhydrous Na₂SO₄, filtered and concentrated under reduced pressure to give a crude product as yellow oil, which was purified using a silica flash cartridge (24 g column) using 10%-40% EtOAc in pet ether as an eluent to afford the product (1.56 g, 82%) as yellow solid. LCMS: [M+2H]⁺=240.21.

Step 5: 3,3-dimethyl-5-(4,4,5,5-tetramethyl-1,3,2-dioxaborolan-2-yl)isoindolin-1-one

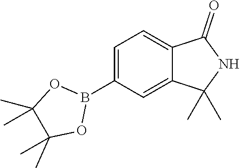 [SMILES: CC1(C)NC(=O)c2ccc(B3OC(C)(C)C(C)(C)O3)cc21]

To a stirred degassed solution of 5-bromo-3,3-dimethylisoindolin-1-one (2.2 g, 9.2 mmol) in 1,4-dioxane (22 mL), KOAc (2.3 g, 23.4 mmol) and B2pin2 (3.12 g, 12.3 mmol) were added under argon atmosphere and degassed with argon for 20 minutes followed by the addition of Pd(dppf)Cl₂.DCM (0.77 g, 0.94 mmol) and heated to 90° C. for 16 h before cooling to RT. The reaction mixture was filtered through Celite® bed which was washed with diethyl ether (250 mL). The filtrate was concentrated under reduced pressure to afford the crude compound as black gum, which was triturated with n-pentane (25 mL) and diethyl ether (10 mL) to afford the product (1.74 g, 66%) as black color solid. TLC: 20% EtOAc: DCM; Rf :0.3

Step 6. 5-(2-Amino-6-fluoro-5-(4-(4-methylpiperazin-1-yl)phenyl)pyridin-3-yl)-3,3-dimethylisoindolin-1-one

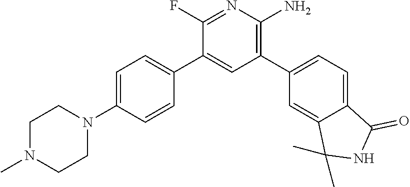 [SMILES: CN1CCN(c2ccc(-c3cc(-c4ccc5c(c4)C(C)(C)NC5=O)c(N)nc3F)cc2)CC1]

A procedure similar to that described in Example 33, Step 2 using 3-bromo-6-fluoro-5-(4-(4-methylpiperazin-1-yl)phenyl)pyridin-2-amine (55 mg, 0.15 mmol) and 3,3-dimethyl-5-(4,4,5,5-tetramethyl-1,3,2-dioxaborolan-2-yl)isoindolin-1-one (65 mg, 0.23 mmol) provided the title compound (27 mg, 38%) as a white solid. ¹H NMR (500 MHz, DMSO-d₆) δ 8.64 (s, 1H), 7.74 (s, 1H), 7.66 (d, J=7.8 Hz, 1H), 7.60 (d, J=10.2 Hz, 1H), 7.57 (dd, J=7.8, 1.34 Hz, 1H), 7.41 (d, J=7.8 Hz, 2H), 6.97 (d, J=8.9 Hz, 2H), 6.13 (s, 2H), 3.13-3.18 (m, 4H), 2.42-2.47 (m, 4H), 2.22 (s, 3H), 1.48 (s, 6H); LCMS: [M+H]⁺=446.05.

### Example 34: 6-(5-(4-((1-acetylpiperidin-4-yl)oxy)phenyl)-2-amino-6-fluoropyridin-3-yl)-3,4-dihydroisoquinolin-1(2H)-one (I-34)

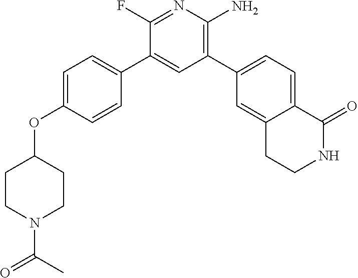 [SMILES: CC(=O)N1CCC(Oc2ccc(-c3cc(-c4ccc5c(c4)CCNC5=O)c(N)nc3F)cc2)CC1]

Step 1: tert-butyl 4-(4-(6-amino-5-chloro-2-fluoropyridin-3-yl)phenoxy)piperidine-1-carboxylate

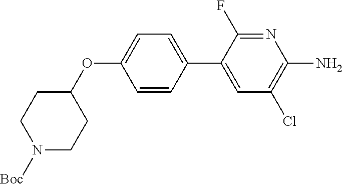 [SMILES: CC(C)(C)OC(=O)N1CCC(Oc2ccc(-c3cc(Cl)c(N)nc3F)cc2)CC1]

To a solution of 5-bromo-3-chloro-6-fluoropyridin-2-amine (100 mg, 0.444 mmol), tert-butyl 4-[4-(tetramethyl-1,3,2-dioxaborolan-2-yl)phenoxy]piperidine-1-carboxylate (192 mg, 0.476 mmol) in 1,4-dioxane (5 mL) was added a solution of Cs₂CO₃ (434 mg, 1.33 mmol) in water (1 mL) at RT. The reaction mixture was degassed under argon for 15 minutes before adding PddppfCl₂ (48.7 mg, 0.067 mmol). Then the reaction mixture was heated at 90° C. for overnight. The reaction mixture was dry loaded onto Celite® and purified by flash chromatography (0-20%, DCM/MeOH) to afford the product (187 mg, 100%) as a dark beige powder which was used for the next step. LCMS: [M+H]⁺=422.35.

Step 2: tert-butyl 4-(4-(6-amino-2-fluoro-5-(1-oxo-1,2,3,4-tetrahydroisoquinolin-6-yl)pyridin-3-yl)phenoxy)piperidine-1-carboxylate

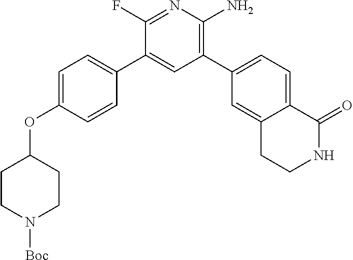 [SMILES: CC(C)(C)OC(=O)N1CCC(Oc2ccc(-c3cc(-c4ccc5c(c4)CCNC5=O)c(N)nc3F)cc2)CC1]

To a solution of tert-butyl 4-(4-(6-amino-5-chloro-2-fluoropyridin-3-yl)phenoxy)piperidine-1-carboxylate (187 mg, 0.443 mmol) and 3,4-dihydro-1(2H)-isoquinolinone-6-boronic acid pinacol ester (182 mg, 0.665 mmol) in 1,4-dioxane (5 ml) was added a solution of K₃PO₄ (282 mg, 1.33 mmol) in water (1 mL) at RT. The reaction mixture was degassed under argon for 15 minutes before adding XPhos Pd G2 (34.9 mg, 0.044 mmol). Then the reaction mixture was heated at 90° C. for 1 hour under microwave. The reaction mixture was dry loaded onto Celite® and purified by flash chromatography (0-10%, DCM/MeOH) to afford the product (199 mg, 84%) as a dark beige powder which was used for the next step. LCMS: [M+H]⁺=533.33.

Step 3: 6-(2-amino-6-fluoro-5-(4-(piperidin-4-yloxy)phenyl)pyridin-3-yl)-3,4-dihydroisoquinolin-1(2H)-one

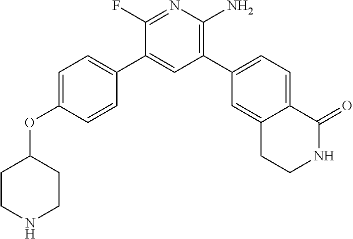 [SMILES: Nc1nc(F)c(-c2ccc(OC3CCNCC3)cc2)cc1-c1ccc2c(c1)CCNC2=O]

To a solution of tert-butyl 4-(4-(6-amino-2-fluoro-5-(1-oxo-1,2,3,4-tetrahydroisoquinolin-6-yl)pyridin-3-yl)phenoxy)piperidine-1-carboxylate (199 mg, 0.374 mmol) in CH₂Cl₂ (5 mL) was added TFA (2 mL, 26.1 mmol) at RT and the reaction mixture was stirred for 1 h. The solvents were removed and the crude mixture was dissolved in MeOH and passed through a PoraPak Rxn CX (20cc-2g) cartridge in a catch & elute method. The solvents were removed and the crude mixture was dissolved in MeOH and passed through a PoraPak Rxn CX (20cc-2g) cartridge in a catch & elute method. The cartridge was washed with MeOH (20 mL), then the solution of product in MeOH was added onto the cartridge and the cartridge was rinsed with MeOH (3×20 mL). The product was released using 20 mL of a solution of ammonia in MeOH (3N) to afford the title compound (124.8 mg, 73%) as a white powder. ¹H NMR (500 MHz, DMSO-d₆) δ 8.00-7.86 (m, 2H), 7.59 (br d, J=10.1 Hz, 1H), 7.50-7.40 (m, 4H), 6.98 (br d, J=8.7 Hz, 2H), 6.21 (s, 2H), 4.47-4.34 (m, 1H), 3.40 (br d, J=3.9 Hz, 2H), 3.01-2.89 (m, 4H), 2.57 (br s, 3H), 1.91 (br d, J=9.7 Hz, 2H), 1.45 (br d, J=9.5 Hz, 2H); [M+H]⁺=433.33.

Step 4: 6-(5-(4-((1-acetylpiperidin-4-yl)oxy)phenyl)-2-amino-6-fluoropyridin-3-yl)-3,4-dihydroisoquinolin-1(2H)-one

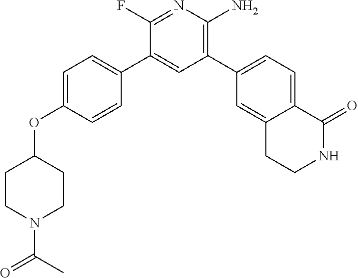 [SMILES: CC(=O)N1CCC(Oc2ccc(-c3cc(-c4ccc5c(c4)CCNC5=O)c(N)nc3F)cc2)CC1]

To a solution of 6-(2-amino-6-fluoro-5-(4-(piperidin-4-yloxy)phenyl)pyridin-3-yl)-3,4-dihydroisoquinolin-1(2H)-one (24.2 mg, 0.056 mmol) and N,N-diisopropylethylamine (0.019 ml, 0.112 mmol) in CH₂Cl₂ (3 mL) was added acetic anhydride (5.29 μl, 0.056 mmol) at RT. The reaction mixture was stirred for 1 h at RT. The reaction mixture was dry loaded and purified by flash chromatography (0-20%, MeOH/DC)] to afford the title compound (24.5 mg, 88%) as a white powder. ¹H NMR (500 MHz, CD3OD) δ 8.03 (d, J=7.9 Hz, 1H), 7.61 (d, J=9.8 Hz, 1H), 7.49 (dd, J=8.1, 1.6 Hz, 1H), 7.48-7.42 (m, 3H), 7.02 (d, J=8.8 Hz, 2H), 4.72-4.63 (m, 1H), 3.87-3.72 (m, 2H), 3.61-3.47 (m, 4H), 3.05 (t, J=6.7 Hz, 2H), 2.13 (s, 3H), 2.07-2.01 (m, 1H), 1.96 (br t, J=4.0 Hz, 1H), 1.85-1.78 (m, 1H), 1.77-1.70 (m, 1H), NH and NH₂ not observed; LCMS: [M+H]⁺=475.24.

### Example 35: 6-(2-amino-6-fluoro-5-(4-((tetrahydro-2H-pyran-4-yl)oxy)phenyl)pyridin-3-yl)-3,4-dihydroisoquinolin-1(2H)-one (I-35)

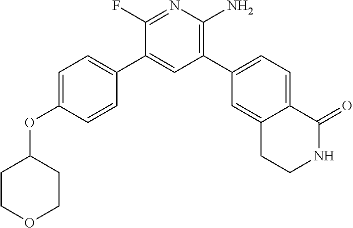 [SMILES: Nc1nc(F)c(-c2ccc(OC3CCOCC3)cc2)cc1-c1ccc2c(c1)CCNC2=O]

To a solution of (4-((tetrahydro-2H-pyran-4-yl)oxy)phenyl)boronic acid (42 mg, 0.189 mmol) and 6-(2-amino-5-bromo-6-fluoropyridin-3-yl)-3,4-dihydroisoquinolin-1(2H)-one (52 mg, 0.155 mmol) in 1,4-dioxane (5 mL) was added a solution of Cs₂CO₃ (151 mg, 0.464 mmol) in water (1 mL) at RT. The reaction mixture was degassed under argon for 15 min. Then PddppfCl2 (16.98 mg, 0.023 mmol) was added and the reaction mixture was heated at 90° C. for overnight. The reaction mixture was dry loaded onto Celite® and purified by flash chromatography (0-10%, DCM/MeOH) to afford the title compound (52.2 mg, 74%) as a beige powder. ¹H NMR (500 MHz, CD₃OD) δ 8.04 (d, J=8.1 Hz, 1H), 7.61 (d, J=9.8 Hz, 1H), 7.52-7.48 (m, 1H), 7.45 (br d, J=7.3 Hz, 3H), 7.01 (d, J=8.7 Hz, 2H), 4.65-4.56 (m, 1H), 4.02-3.91 (m, 2H), 3.60 (ddd, J=11.6, 8.7, 3.1 Hz, 2H), 3.57-3.52 (m, 2H), 3.35 (s, 2H), 3.10-3.01 (m, 2H), 2.10-2.00 (m, 2H), 1.80-1.68 (m, 2H), NH and NH₂ not observed; LCMS: [M+H]⁺=434.31.

### Example 36: 5-(2-amino-6-fluoro-5-(4-((1R,5S)-3-methyl-3-azabicyclo[3.1.0]hexan-1-yl)phenyl)pyridin-3-yl)-3,3-dimethylisoindolin-1-one (I-36)

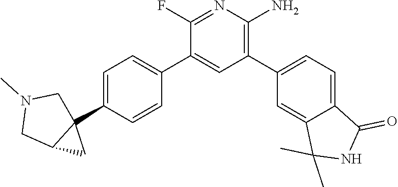

Step 1: 5-(2-amino-6-fluoropyridin-3-yl)-3,3-dimethylisoindolin-1-one

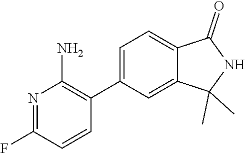 [SMILES: CC1(C)NC(=O)c2ccc(-c3ccc(F)nc3N)cc21]

A procedure similar to that described in Example 1, General Method A, heating a sealed, degassed mixture of 3,3-dimethyl-5-(4,4,5,5-tetramethyl-1,3,2-dioxaborolan-2-yl)isoindolin-1-one (254 mg, 0.884 mmol) (preparation described in Example 33, Step 5), K₃PO₄ (469 mg, 2.21 mmol), 3-chloro-6-fluoropyridin-2-amine (108 mg, 0.737 mmol), XPhos Pd G2 (53.7 mg, 0.068 mmol), H₂O (6 mL) and MeCN (9 mL) in a microwave reactor at 100° C. for 2 h afforded, after purification by flash chromatography (25 g SiO₂, using MeOH in CH₂Cl₂) the product (214.0 mg, 97% yield based on purity of 91%). LCMS: [M+H]⁺=272.22.

Step 2: 5-(2-amino-5-bromo-6-fluoropyridin-3-yl)-3,3-dimethylisoindolin-1-one

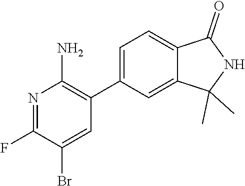 [SMILES: CC1(C)NC(=O)c2ccc(-c3cc(Br)c(F)nc3N)cc21]

To a stirred mixture of 5-(2-amino-6-fluoropyridin-3-yl)-3,3-dimethylisoindolin-1-one (214 mg, 0.718 mmol, 91%) in MeCN (10 mL), was added NBS (134 mg, 0.754 mmol) in one portion at 0° C. The reaction was allowed to warm slowly and stirred at RT overnight. The reaction mixture was concentrated and purified by flash chromatography (25 g SiO₂, using MeOH in CH₂Cl₂) to afford the product as a light brown foam (298 mg, quant yield). LCMS: [M+H]⁺=350.14/352.17.

Step 3: 5-(2-amino-6-fluoro-5-(4,4,5,5-tetramethyl-1,3,2-dioxaborolan-2-yl)pyridin-3-yl)-3,3-dimethylisoindolin-1-one

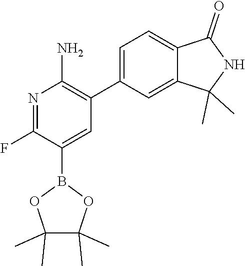 [SMILES: CC1(C)NC(=O)c2ccc(-c3cc(B4OC(C)(C)C(C)(C)O4)c(F)nc3N)cc21]

A vial was charged with 5-(2-amino-5-bromo-6-fluoropyridin-3-yl)-3,3-dimethylisoindolin-1-one (298 mg, 0.808 mmol), KOAc (159 mg, 1.62 mmol), PCy3 (22.67 mg, 0.081 mmol), B₂pin₂ (205 mg, 0.808 mmol) and Pd₂dba₃ (74.0 mg, 0.081 mmol). Anhydrous 1,4-dioxane (10 mL) was added and the suspension was degassed with a stream of N₂. The reaction mixture was then heated sealed in an oil bath at 90° C. for 18 h and later at 110° C. for 2 h. Additional KOAc (159 mg, 1.62 mmol), Pd₂dba₃ (74.0 mg, 0.081 mmol), B₂pin₂ (205 mg, 0.808 mmol) and PCy₃ (22.7 mg, 0.081 mmol) was then added; degassing was repeated and stirring was continued with heating at 110° C. for 2.5 h. The crude product (89% by LCMS) was used without further modification as the 1,4-dioxane mixture in the following step.

Step 4: 5-(2-amino-6-fluoro-5-(4-((1R,5S)-3-methyl-3-azabicyclo[3.1.0]hexan-1-yl)phenyl)pyridin-3-yl)-3,3-dimethylisoindolin-1-one

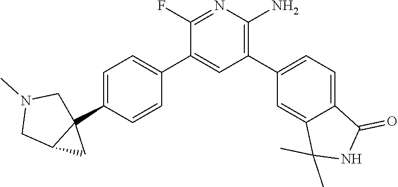

A procedure similar to that used in Example 1, General Method A using 5-(2-amino-6-fluoro-5-(4,4,5,5-tetramethyl-1,3,2-dioxaborolan-2-yl)pyridin-3-yl)-3,3-dimethylisoindolin-1-one (124 mg, 0.278 mmol, 89% purity), in 1,4-dioxane (3.86 mL), PdCl₂dppf (20.3 mg, 0.028 mmol), (1R,5S)-1-(4-bromophenyl)-3-methyl-3-azabicyclo[3.1.0]hexane (70 mg, 0.278 mmol), Cs₂CO₃ (271 mg, 0.833 mmol), H₂O (5 mL) and DME (6 mL) and heating in a microwave reactor at 90° C. for 1.5 h afforded the title compound as an off-white solid (39.0 mg, 31% yield based on purity of 99%). ¹H NMR (500 MHz, DMSO-d₆) δ 8.65 (s, 1H), 7.75 (s, 1H), 7.62-7.68 (m, 2H), 7.58 (dd, J=7.8, 1.2 Hz, 1H), 7.48 (d, J=7.3 Hz, 2H), 7.19 (d, J=8.3 Hz, 2H), 6.25 (s, 2H), 3.17-3.30 (m, 2H), 3.00 (br d, J=8.2 Hz, 1H), 2.42 (br s, 1H), 2.28-2.36 (m, 3H), 1.76-1.92 (m, 1H), 1.49 (s, 6H), 1.36 (t, J=4.1 Hz, 1H), 0.78 (br dd, J=7.6, 3.6 Hz, 1H). LCMS: [M+H]⁺=443.11.

### Example 37: 5-(2-amino-6-fluoro-5-(4-(1-methyl-5,6-dihydro-1,2,4-triazin-4(1H)-yl)phenyl)pyridin-3-yl)-3,3-dimethylisoindolin-1-one 2,2,2-trifluoroacetate (I-37)

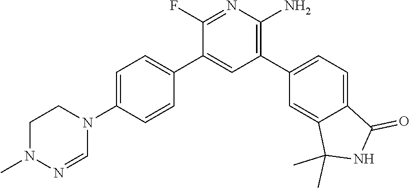 [SMILES: CN1CCN(c2ccc(-c3cc(-c4ccc5c(c4)C(C)(C)NC5=O)c(N)nc3F)cc2)C=N1]

A procedure similar to that described in Example 1, General Method A using 5-(2-amino-6-fluoro-5-(4,4,5,5-tetramethyl-1,3,2-dioxaborolan-2-yl)pyridin-3-yl)-3,3-dimethylisoindolin-1-one (123 mg, 0.275 mmol, preparation described in Example 33, Step 5) in 1,4-dioxane (3.83 mL), Pd (amphos)Cl₂ (19.5 mg, 0.028 mmol), K₃PO₄ (175 mg, 0.826 mmol), 4-(4-bromophenyl)-1-methyl-1,4,5,6-tetrahydro-1,2,4-triazine (70 mg, 0.275 mmol) (preparation described in Example 31, Step 8), H₂O (1.4 mL) and additional 1,4-dioxane (6 mL) afforded the TFA salt of the title compound as a light beige sticky solid (42.0 mg, 27%). ¹H NMR (500 MHz, CD₃OD) δ 8.64 (s, 1H), 7.86 (d, J=7.8 Hz, 1H), 7.68-7.76 (m, 4H), 7.63 (dd, J=7.8, 1.5 Hz, 1H), 7.49-7.54 (m, 2H), 4.05-4.12 (m, 2H), 3.40-3.44 (m, 2H), 2.90 (s, 3H), 1.58-1.63 (m, 6H); LCMS: [M+H]⁺=445.30.

### Example 38: (R)-6-(2-amino-6-fluoro-5-(4-(hexahydropyrazino[2,1-c][1,4]oxazin-8(1H)-yl)phenyl)pyridin-3-yl)-3,4-dihydroisoquinolin-1(2H)-one (I-38)

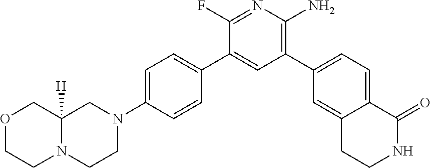 [SMILES: Nc1nc(F)c(-c2ccc(N3CCN4CCOC[C@H]4C3)cc2)cc1-c1ccc2c(c1)CCNC2=O]

Step 1: (R)-8-(4-iodophenyl)octahydropyrazino[2, 1-c][1,4]oxazine

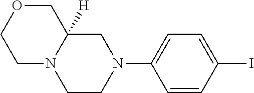 [SMILES: Ic1ccc(N2CCN3CCOC[C@H]3C2)cc1]

The product was prepared by a method similar to Example 23, Step 1 using (R)-octahydro-pyrazino[2,1-c][1,4]oxazine ·2HCl (381 mg, 1.771 mmol), K₃PO₄ (1896 mg, 8.93 mmol), DMF (20 mL), 1,4-diiodobenzene (508 mg, 1.54 mmol), Cul (64.5 mg, 0.339 mmol) and (R)-(+)-1,1′-bi-2-naphthol (97 mg, 0.339 mmol) to afford the product as a light tan solid (279 mg, 53%). LCMS: [M+H]⁺=345.17.

Step 2: (R)-6-(2-amino-6-fluoro-5-(4-(hexahydropyrazino[2,1-c][1,4]oxazin-8(1H)-yl)phenyl)pyridin-3-yl)-3,4-dihydroisoquinolin-1(2H)-one

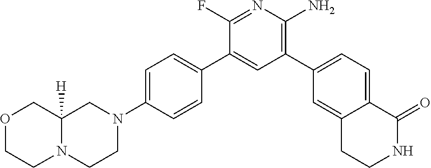 [SMILES: Nc1nc(F)c(-c2ccc(N3CCN4CCOC[C@H]4C3)cc2)cc1-c1ccc2c(c1)CCNC2=O]

A procedure similar to Example 1, General Method A by heating a sealed and degassed mixture of (R)-8-(4-iodophenyl)octahydropyrazino[2,1-c][1,4]oxazine (80 mg, 0.232 mmol), PdCl₂dppf (20.3 mg, 0.028 mmol), Cs₂CO₃ (227 mg, 0.697 mmol), H₂O (3 mL), and 6-(2-amino-6-fluoro-5-(4,4,5,5-tetramethyl-1,3,2-dioxaborolan-2-yl)pyridin-3-yl)-3,4-dihydroisoquinolin-1(2H)-one (111 mg, 0.232 mmol, 18.5 mg/mL, 80% purity) in 1,4-dioxane (6.02 mL) in a microwave reactor at 90° C. afforded, after purification by flash chromatography (25 g SiO₂, eluting with MeOH in CH₂Cl), the title compound as a beige solid (60.0 mg, 52% yield based on purity of 95%). ¹H NMR (500 MHz, DMSO-d₆) δ 7.88-7.97 (m, 1H), 7.59 (d, J=10.3 Hz, 1H), 7.44-7.49 (m, 2H), 7.41 (d, J=7.8 Hz, 2H), 6.98 (d, J=9.1 Hz, 2H), 6.18 (s, 2H), 3.77 (td, J=10.6, 2.6 Hz, 2H), 3.67 (br d, J=11.6 Hz, 1H), 3.50-3.59 (m, 2H), 3.35-3.45 (m, 3H), 3.13-3.21 (m, 1H), 2.96 (t, J=6.5 Hz, 2H), 2.73-2.86 (m, 2H), 2.69 (br d, J=11.7 Hz, 1H), 2.17-2.34 (m, 4H); LCMS: [M+H]⁺=474.41.

### Example 39: (S)-6-(2-amino-6-fluoro-5-(4-(hexahydropyrazino[2,1-c][1,4]oxazin-8(1H)-yl)phenyl)pyridin-3-yl)-3,4-dihydroisoquinolin-1(2H)-one (I-39)

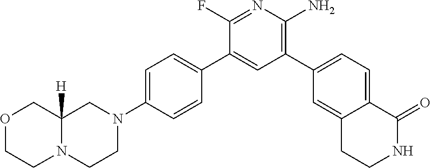 [SMILES: Nc1nc(F)c(-c2ccc(N3CCN4CCOC[C@@H]4C3)cc2)cc1-c1ccc2c(c1)CCNC2=O]

Step 1: (S)-8-(4-iodophenyl)octahydropyrazino[2,1-c][1,4]oxazine

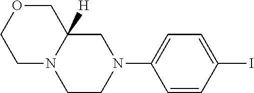 [SMILES: Ic1ccc(N2CCN3CCOC[C@@H]3C2)cc1]

A procedure similar to that described in Example 23, Step 1 using (S)-octahydropyrazino[2,1-c][1,4]oxazine·2HCl (376 mg, 1.75 mmol), K₃PO₄ (1871 mg, 8.82 mmol) 1,4-diiodobenzene (501 mg, 1.52 mmol), Cul (63.7 mg, 0.334 mmol) and (R)-(+)-1,1′-bi-2-naphthol (96 mg, 0.334 mmol) in DMF (20 mL) afforded the product as a rust orange solid (128 mg, 22% yield based on purity of 89%). LCMS: [M+H]⁺=345.17.

Step 2: (S)-6-(2-amino-6-fluoro-5-(4-(hexahydropyrazino[2,1-c][1,4]oxazin-8(1H)-yl)phenyl)pyridin-3-yl)-3,4-dihydroisoquinolin-1(2H)-one

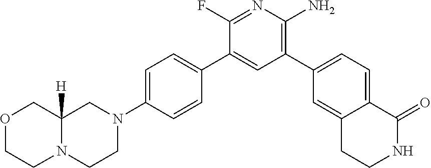 [SMILES: Nc1nc(F)c(-c2ccc(N3CCN4CCOC[C@@H]4C3)cc2)cc1-c1ccc2c(c1)CCNC2=O]

A procedure similar to that described in Example 1, General Method A, heating a mixture of (S)-8-(4-iodophenyl)octahydropyrazino[2,1-c][1,4]oxazine (80 mg, 0.233 mmol), PdCl₂dppf (20.3 mg, 0.028 mmol), Cs₂CO₃ (227 mg, 0.698 mmol), H₂O (3 mL) and 6-(2-amino-6-fluoro-5-(4,4,5,5-tetramethyl-1,3,2-dioxaborolan-2-yl)pyridin-3-yl)-3,4-dihydroisoquinolin-1(2H)-one (89 mg, 18.5 mg/mL, 0.233 mmol, 80%) in 1,4-dioxane (6.03 mL) afforded, after the mixture was concentrated under reduced pressure and purified by flash chromatography (25 g SiO₂, using MeOH in CH₂Cl₂) followed by a filtration through a Waters PoraPak CX column the title compound as a beige solid (24 mg, 22% yield based on purity of 99%). ¹H NMR (500 MHz, DMSO-d₆) δ 7.94 (br s, 1H), 7.91 (d, J=7.8 Hz, 1H), 7.59 (d, J=10.2 Hz, 1H), 7.44-7.49 (m, 2H), 7.41 (br d, J=8.0 Hz, 2H), 6.98 (d, J=8.9 Hz, 2H), 6.18 (s, 2H), 3.71-3.82 (m, 2H), 3.67 (br d, J=11.6 Hz, 1H), 3.50-3.59 (m, 2H), 3.35-3.46 (m, 2H), 3.10-3.25 (m, 1H), 2.96 (br t, J=6.5 Hz, 2H), 2.72-2.86 (m, 2H), 2.65-2.71 (m, 1H), 2.17-2.34 (m, 4H); LCMS: [M+H]⁺=474.11.

### Example 40: 6-(2-amino-6-fluoro-5-(3-fluoro-4-(4-methylpiperazin-1-yl)phenyl)pyridin-3-yl)-3,4-dihydroisoquinolin-1(2H)-one (I-40)

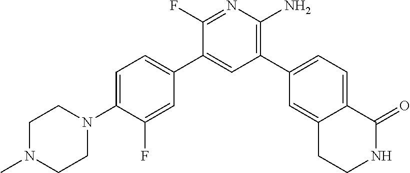 [SMILES: CN1CCN(c2ccc(-c3cc(-c4ccc5c(c4)CCNC5=O)c(N)nc3F)cc2F)CC1]

A procedure analogous to Example 8 Step 4 using 6-(2-amino-5-bromo-6-fluoropyridin-3-yl)-3,4-dihydroisoquinolin-1(2H)-one (100 mg, 0.30 mmol) and 3-fluoro-4-(4-methylpiperazino)phenylboronic acid pinacol ester (100 mg, 0.31 mmol) provided the title compound (26 mg, 18%) as a grey solid. ¹H NMR (500 MHz, DMSO-d₆) δ 7.93 (br s, 1H), 7.90 (d, J=8.0 Hz, 1H), 7.65 (d, J=10.2 Hz, 1H), 7.47 (dd, J=8.0, 1.5 Hz, 1H), 7.45 (s, 1H), 7.35 (br d, J=14.6 Hz, 1H), 7.31 (br d, J=8.4 Hz, 1H), 7.05 (t, J=9.0 Hz, 1H), 6.30 (s, 2H), 3.40 (td, J=6.5, 2.7 Hz, 2H), 3.03 (br t, J=4.3 Hz, 4H), 2.95 (t, J=6.5 Hz, 2H), 2.47 (br s, 4H), 2.22 (s, 3H); LCMS: [M+H]⁺=450.26.

### Example 41: 6-(2-amino-5-(4-(4-ethylpiperazin-1-yl)phenyl)-6-fluoropyridin-3-yl)-3,4-dihydroisoquinolin-1(2H)-one (I-41)

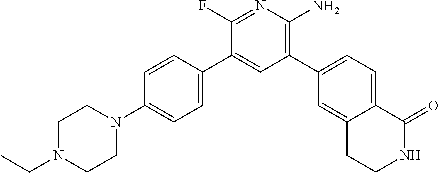 [SMILES: CCN1CCN(c2ccc(-c3cc(-c4ccc5c(c4)CCNC5=O)c(N)nc3F)cc2)CC1]

A procedure analogous to Example 8, Step 4 using 6-(2-amino-5-bromo-6-fluoropyridin-3-yl)-3,4-dihydroisoquinolin-1(2H)-one (100 mg, 0.30 mmol) and (4-(4-ethylpiperazin-1-yl)phenyl)boronic acid pinacol ester (99 mg, 0.31 mmol) provided the title compound (25 mg, 18%) as a grey solid. ¹H NMR (500 MHz, DMSO-d₆) δ 7.95 (br s, 1H), 7.92 (d, J=8.0 Hz, 1H), 7.60 (d, J=10.3 Hz, 1H), 7.48 (dd, J=8.0, 1.5 Hz, 1H), 7.46 (s, 1 H), 7.41 (d, J=7.8 Hz, 2H), 6.98 (d, J=8.9 Hz, 2H), 6.18 (s, 2H), 3.42 (td, J=6.5, 2.6 Hz, 2H), 3.14-3.20 (m, 4H), 2.97 (t, J=6.5 Hz, 2H), 2.51 (br dd, J=3.6, 1.6 Hz, 4H), 2.38 (q, J =7.2 Hz, 2H), 1.05 (t, J=7.2 Hz, 3H); LCMS: [M+1]⁺=446.27.

### Example 42: 6-(2-amino-6-fluoro-5-(4-(4-isopropylpiperazin-1-yl)phenyl)pyridin-3-yl)-3,4-dihydroisoquinolin-1(2H)-one (I-42)

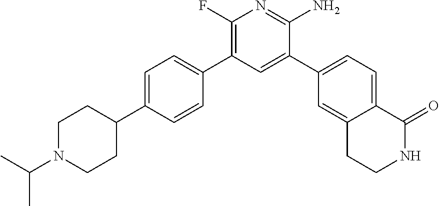 [SMILES: CC(C)N1CCC(c2ccc(-c3cc(-c4ccc5c(c4)CCNC5=O)c(N)nc3F)cc2)CC1]

In a microwave vial with magnetic stir bar was placed 6-(2-amino-5-bromo-6-fluoropyridin-3-yl)-3,4-dihydroisoquinolin-1(2H)-one (100 mg, 0.297 mmol), Cs₂CO₃ (291 mg, 0.892 mmol), DME (4 mL) and water (2 mL). The flask was sealed, then heated to 90° C. for 1 h in the microwave. The reaction was loaded onto Celite® and purified by reverse phase chromatography (C₁₈, MeCN—H₂O) followed by silica gel chromatography (SiO₂, 100% DCM to 95-5-1 DCM/MeOH/NH₄OH) to give the title compound (19 mg, 14%) as a grey solid. ¹H NMR (500 MHz, DMSO-d₆) δ 7.86 (br s, 1H), 7.83 (d, J=8.0 Hz, 1H), 7.51 (d, J=10.2 Hz, 1H), 7.38-7.42 (m, 1H), 7.37 (s, 1H), 7.33 (d, J=7.8 Hz, 2H), 6.89 (d, J=8.9 Hz, 2H), 6.10 (s, 2H), 3.34 (td, J=6.5, 2.7 Hz, 2H), 3.05-3.10 (m, 4H), 2.88 (t, J=6.5 Hz, 2H), 2.56-2.64 (m, 1H), 2.48-2.53 (m, 4H), 0.94 (d, J=6.5 Hz, 6H); LCMS: [M+1]⁺=460.27.

### Example 43: 6-(2-amino-5-(4-(1-(2,2-difluoroethyl)piperidin-4-yl)phenyl)-6-fluoropyridin-3-yl)-3,4-dihydroisoquinolin-1(2H)-one (I-43)

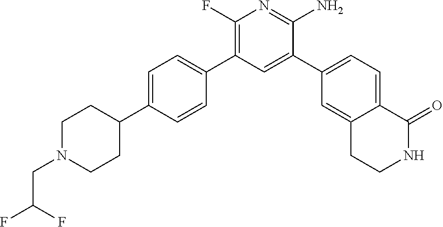 [SMILES: Nc1nc(F)c(-c2ccc(C3CCN(CC(F)F)CC3)cc2)cc1-c1ccc2c(c1)CCNC2=O]

Step 1: 4-(4-bromophenyl)-1-(2, 2-difluoroethyl)piperidine

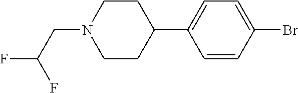 [SMILES: FC(F)CN1CCC(c2ccc(Br)cc2)CC1]

To a slurry of 4-(4-bromophenyl)piperidine (400 mg, 1.67 mmol) in DMF (5 mL) at RT, was added 2,2-difluoroethyl trifluoromethanesulfonate (0.444 mL, 3.33 mmol), followed by Et₃N (2.32 mL, 16.7 mmol) and the reaction mixture was stirred at RT for 10 min. The reaction mixture was concentrated to dryness and the residue was partitioned between CH₂Cl₂ and water. The layers were separated and the aqueous layer was extracted with CH₂Cl₂ (x2). The combined organic extracts were dried and concentrated onto Celite®. Silica gel chromatography (eluting with CH₂Cl₂ containing 0-0.5% MeOH and 0-0.05% NH₄OH) afforded the product as a pale yellow oil (384 mg, 76%). LCMS: [M+H]⁺=304.37.

Step 2: 5-bromo-6-fluoro-3-iodopyridin-2-amine:

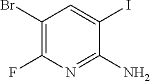

To a solution of 5-bromo-6-fluoropyridin-2-amine (40 g, 210.5 mmol) in acetic acid (280 mL), was added N-iodo succinimide (47.4 g, 210.5 mmol) and TFA (2.6 mL) and the mixture was stirred at RT for 2.5 h, then poured into ice water and neutralized with aqueous ammonia. The resulting precipitate was filtered, washed with ice water, and dried over under vacuum to afford the product (60 g, 90%) as a yellow solid. LCMS: [M+H]⁺=316.8.

Step 3: 6-(2-amino-5-bromo-6-fluoropyridin-3-yl)-3,4-dihydroisoquinolin-1(2H)-one:

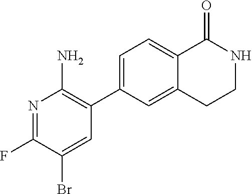 [SMILES: Nc1nc(F)c(Br)cc1-c1ccc2c(c1)CCNC2=O]

To a degassed suspension of 5-bromo-6-fluoro-3-iodopyridin-2-amine (40 g, 126.6 mmol), 6-(4,4,5,5-tetramethyl-1,3,2-dioxaborolan-2-yl)-3,4-dihydroisoquinolin-1(2H)-one (38.15 g, 139.3 mmol) and K₂CO₃ (34.95 g, 253.2 mmol) in a mixture of DMF (400 mL) and H₂O (80 mL) was added Pd(PPh₃)₂Cl₂, (8.87 g, 12.6 mmol). The reaction mixture was heated to 80° C. for 16 h before cooling to RT. The reaction mixture was poured into ice water and extracted with EtOAc (2×500 mL), washed with water (500 mL) and brine (500 mL), dried over Na₂SO₄ and concentrated under reduced pressure to give crude residue which was purified by column chromatography (silica gel, 100-200 mesh) using eluent 60-80% EtOAc in petroleum ether to afford the product (23 g, 54%) as a brown solid. LCMS: [M+H]⁺=336.04

Step 4: 6-(2-amino-6-fluoro-5-(4,4,5,5-tetramethyl-1,3,2-dioxaborolan-2-yl)pyridin-3-yl)-3,4-dihydroisoquinolin-1(2H)-one:

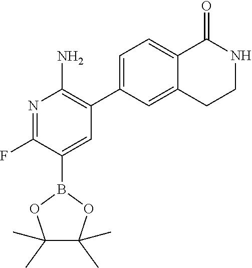 [SMILES: CC1(C)OB(c2cc(-c3ccc4c(c3)CCNC4=O)c(N)nc2F)OC1(C)C]

To a degassed suspension of 6-(2-amino-5-bromo-6-fluoropyridin-3-yl)-3,4-dihydroisoquinolin-1(2H)-one (10 g, 29.8 mmol), 4,4,4′,4′,5,5,5′,5′-octamethyl-2,2′-bi(1,3,2-dioxaborolane) (22.7 g, 89.5 mmol), and KOAc (10.23 g, 104.4 mmol) in toluene (200 mL) was added a Pd₂(dba)₃ (1.64 g, 1.8 mmol) and X-PHOS (1.42 g, 3 mmol). The reaction mixture was heated to 110° C. for 3 h under argon atmosphere before cooling to RT. The reaction mixture was filtered through a Celite® bed, which was washed with 20% MeOH in DCM (500 mL). Then, the filtrate was concentrated under reduced pressure to give residue; which was purified by washed with EtOAc (100 mL) to afford the product (9 g, 79%) as a pale brown solid. LCMS: [M+H]⁺=384.27

Step 5: 6-(2-amino-5-(4-(1-(2, 2-difluoroethyl)piperidin-4-yl)phenyl)-6-fluoropyridin-3-yl)-3,4-dihydroisoquinolin-1(2H)-one

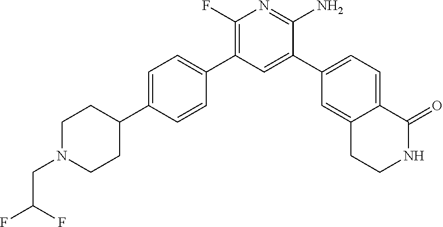 [SMILES: Nc1nc(F)c(-c2ccc(C3CCN(CC(F)F)CC3)cc2)cc1-c1ccc2c(c1)CCNC2=O]

A microwave vial charged with 6-(2-amino-6-fluoro-5-(4,4,5,5-tetramethyl-1,3,2-dioxaborolan-2-yl)pyridin-3-yl)-3,4-dihydroisoquinolin-1(2H)-one (70.9 mg, 0.148 mmol), 4-(4-bromophenyl)-1-(2,2-difluoroethyl)piperidine (30 mg, 0.099 mmol, prepared in Step 1), Cs₂CO₃ (96 mg, 0.296 mmol), [1,1′-bis(diphenylphosphino)ferrocene]dichloropalladium(II) (10.82 mg, 0.015 mmol), 1,2-dimethoxyethane (3.25 mL) and H₂O (2.5 mL) was flushed with Argon. The vial was sealed and heated in a microwave reactor at 90° C. for 1 h. The reaction was partitioned between EtOAc and brine. The layers were separated and the aqueous layer was extracted with EtOAc (x 2). The combined organic extracts were dried and concentrated onto Celite®. Reverse phase chromatography (C18; 0-35% acetonitrile/water) followed by filtration through a Waters PoraPak CX column afforded the title compound as a beige solid (15 mg, 30% yield based on purity of 95%). ¹H NMR (500 MHz, DMSO-d₆) δ 7.99-7.87 (m, 2H), 7.68-7.60 (m, 1H), 7.55-7.42 (m, 4H), 7.35-7.26 (m, 2H), 6.34-6.27 (m, 2H), 6.26-5.99 (m, 1H), 3.46-3.39 (m, 2H), 3.05-2.99 (m, 2H), 2.98-2.92 (m, 2H), 2.81-2.71 (m, 2H), 2.58-2.55 (m, 1H), 2.32-2.24 (m, 2H), 1.80-1.73 (m, 2H), 1.72-1.64 (m, 2H). LCMS: [M+H]⁺=481.52

### Example 44: 6-(2-amino-6-fluoro-5-(4-((1-methylpiperidin-4-yl)oxy)phenyl)pyridin-3-yl)-3,4-dihydroisoquinolin-1(2H)-one (I-44)

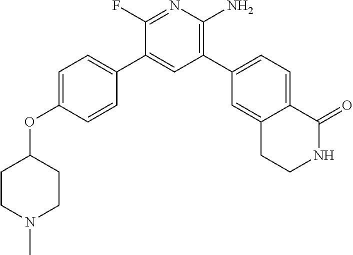 [SMILES: CN1CCC(Oc2ccc(-c3cc(-c4ccc5c(c4)CCNC5=O)c(N)nc3F)cc2)CC1]

To a solution of 1-methyl-4-(4-(4,4,5,5-tetramethyl-1,3,2-dioxaborolan-2-yl)phenoxy)piperidine (61.7 mg, 0.195 mmol) and 6-(2-amino-5-bromo-6-fluoropyridin-3-yl)-3,4-dihydroisoquinolin-1(2H)-one (54.5 mg, 0.162 mmol) in 1,4-Dioxane (3 ml) was added a solution of Cs₂CO₃ (158 mg, 0.486 mmol) in water (1 mL) at RT and the reaction mixture was degassed under argon for 15 minutes. Then PddppfCl2 (17.79 mg, 0.024 mmol) was added and the reaction mixture was heated at 90° C. for 6 hours. The reaction mixture was dry loaded onto Celite® and purified by flash chromatography (0-20%, DCM/MeOH) to afford the title compound (66.6 mg, 87%) as a light brown powder. ¹H NMR (500 MHz, DMSO-d₆) δ 7.93 (br s, 1H), 7.90 (d, J=7.9 Hz, 1H), 7.59 (d, J=10.1 Hz, 1H), 7.46 (br d, J=9.3 Hz, 4H), 7.00 (d, J=8.8 Hz, 2H), 6.22 (s, 2H), 4.48-4.39 (m, 1H), 3.43-3.38 (m, 2H), 2.95 (s, 2H), 2.80-2.68 (m, 2H), 2.43-2.17 (m, 5H), 2.02-1.93 (m, 2H), 1.70 (br s, 2H); LCMS: [M+H]⁺=447.33.

### Example 45: 6-(2-amino-5-(4-((2-(dimethylamino)ethyl)(methyl)amino)phenyl)-6-fluoropyridin-3-yl)-3,4-dihydroisoquinolin-1(2H)-one (I-45)

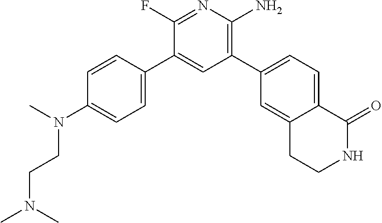 [SMILES: CN(C)CCN(C)c1ccc(-c2cc(-c3ccc4c(c3)CCNC4=O)c(N)nc2F)cc1]

To a solution of 6-(2-amino-5-bromo-6-fluoropyridin-3-yl)-3,4-dihydroisoquinolin-1(2H)-one (63.5 mg, 0.189 mmol) and N1,N1,N2-trimethyl-N2-(4-(4,4,5,5-tetramethyl-1,3,2-dioxaborolan-2-yl)phenyl)ethane-1,2-diamine (63.2 mg, 0.208 mmol) in 1,4-dioxane (3 mL) was added a solution of Cs₂CO₃ (185 mg, 0.567 mmol) in water (1 mL) at RT and the reaction mixture was degassed under argon for 15 minutes. Then PddppfCl₂ (20.73 mg, 0.028 mmol) was added and the reaction mixture was heated at 90° C. for 6 h. The reaction mixture was dry loaded onto Celite® and purified by flash chromatography (0-20%, DCM/MeOH) to afford the title compound (70.5 mg, 82%) as a light brown powder. ¹H NMR (500 MHz, DMSO-d₆) δ 7.93 (br s, 1H), 7.90 (d, J=7.9 Hz, 1H), 7.56 (d, J=10.1 Hz, 1H), 7.49-7.42 (m, 2H), 7.36 (br d, J=8.1 Hz, 2H), 6.73 (d, J=8.9 Hz, 2H), 6.12 (s, 2H), 3.46 (br t, J=7.1 Hz, 2H), 3.40 (br d, J=2.4 Hz, 2H), 2.95 (br t, J=6.5 Hz, 2H), 2.92 (s, 3H), 2.48-2.42 (m, 2H), 2.25 (s, 6H); LCMS: [M+H]⁺=434.31.

### Example 46: (R)-6-(2-amino-6-fluoro-5-(4-(2-methylpyrrolidin-1-yl)phenyl)pyridin-3-yl)-3,4-dihydroisoquinolin-1(2H)-one (I-46)

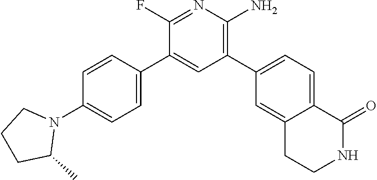 [SMILES: C[C@@H]1CCCN1c1ccc(-c2cc(-c3ccc4c(c3)CCNC4=O)c(N)nc2F)cc1]

Step 1: (R)-1-(4-chlorophenyl)-2-methylpyrrolidine

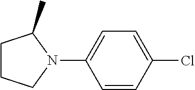

A 30 mL vial was charged with (R)-2-methylpyrrolidine hydrochloride (0.30 g, 2.5 mmol), 1-chloro-4-iodobenzene (0.74 g, 3.1 mmol), tris(dibenzylideneacetone)dipalladium (0) (0.068 g, 0.074 mmol), 4,5-bis(diphenylphosphino)-9,9-dimethylxanthene (0.13 g, 0.22 mmol) and Cs₂CO₃ (4.0 g, 12 mmol). The vial was sealed with a cap and septum and then the reaction vial was evacuated and backfilled with nitrogen. Toluene (8 mL) was added and the reaction vial was evacuated and backfilled with nitrogen an additional time. The reaction was heated conventionally at 100° C. for 18 h. The reaction mixture was cooled to RT and partitioned between EtOAc and water. The layers were separated and the aqueous layer was extracted with additional EtOAc. The combined organic extracts were dried and concentrated onto Celite®. Flash chromatography (1-10% EtOAc/hexanes) afforded the product (0.18 g, 37%). LCMS: [M+H]⁺=196.1.

Step 2: (R)-2-methyl-1-(4-(4,4,5,5-tetramethyl-1,3,2-dioxaborolan-2-yl)phenyl)pyrrolidine

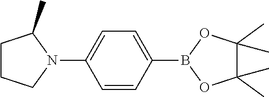 [SMILES: C[C@@H]1CCCN1c1ccc(B2OC(C)(C)C(C)(C)O2)cc1]

A 30 mL vial was charged with (R)-1-(4-chlorophenyl)-2-methylpyrrolidine (0.18 g, 0.91 mmol), bis(pinacolato)diboron (0.35 g, 1.4 mmol), KOAc (0.18 g, 1.8 mmol) and XPhos Pd G2 (0.054 g, 0.068 mmol). The vial was evacuated and backfilled with nitrogen. 1,4-dioxane (8 mL) was added and the vial was evacuated and backfilled an additional time. The reaction mixture was then heated to 110° C. in an aluminum block for 18 h. The reaction mixture was concentrated onto Celite® and purified by flash chromatography (0-8% EtOAc/hexanes) to afford the product (14 mg, 52%). LCMS: [M+H]⁺=288.1.

Step 3: (R)-3-chloro-6-fluoro-5-(4-(2-methylpyrrolidin-1-yl)phenyl)pyridin-2-amine

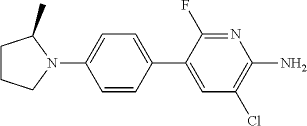 [SMILES: C[C@@H]1CCCN1c1ccc(-c2cc(Cl)c(N)nc2F)cc1]

A 30 mL was charged with 5-bromo-3-chloro-6-fluoropyridin-2-amine (0.050 g, 0.22 mmol), (R)-2-methyl-1-(4-(4,4,5,5-tetramethyl-1,3,2-dioxaborolan-2-yl)phenyl)pyrrolidine (0.070 g, 0.24 mmol) and [1,12-bis(diphenylphosphino)ferrocene]dichloropalladium (II) DCM complex (0.018 g, 0.022 mmol). The vial was sealed with a cap and septum and the reaction vessel was evacuated and backfilled with nitrogen. 1,4-Dioxane (2 mL) and 2 M aqueous Na₂CO₃ (0.33 mL, 0.66 mmol) were added and the reaction vessel was evacuated and backfilled with nitrogen an additional time. The reaction mixture was heated at 90° C. for 18 h in an aluminum block. The reaction mixture was concentrated onto Celite® and purified by flash chromatography (0-10% EtOAc/hexanes) to afford the product (23 mg, 34%). LCMS: [M+H]⁺=306.3.

Step 4: (R)-6-(2-amino-6-fluoro-5-(4-(2-methylpyrrolidin-1-yl)phenyl)pyridin-3-yl)-3,4-dihydroisoquinolin-1(2H)-one

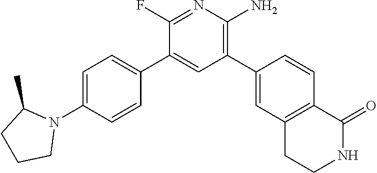 [SMILES: C[C@@H]1CCCN1c1ccc(-c2cc(-c3ccc4c(c3)CCNC4=O)c(N)nc2F)cc1]

A 30 mL vial was charged with 3,4-dihydro-1(2H)-isoquinolinone-6-boronic acid pinacol ester (0.024 g, 0.086 mmol), (R)-3-chloro-6-fluoro-5-(4-(2-methylpyrrolidin-1-yl)phenyl)pyridin-2-amine (0.023 g, 0.075 mmol) and XPhos Pd G2 (0.0059 mg, 0.0075 mmol). The vial was sealed with a cap and septum and the reaction vessel was evacuated and backfilled with nitrogen. 1,4-Dioxane (1.5 mL) and aqueous K₃PO₄ (0.17 mL of a 1.3 M solution, 0.23 mmol) were added and the reaction vessel was evacuated and backfilled with nitrogen an additional time. The reaction mixture was heated at 100° C. for 18 h in an aluminum block. The reaction mixture was concentrated onto Celite® and purified by flash chromatography (0.5-10% DCM/MeOH+1% NH₄OH). The product containing fractions were concentrated and further purified by reverse phase chromatography (Biotage SNAP C18; 5-60% MeCN/water+0.1% Formic Acid). Isolation of the title compound by a catch and release procedure using Biotage SCX2 silica gel afforded the title compound (8 mg, 23%). ¹H NMR (500 MHz DMSO-d₆) δ 7.8-7.9 (m, 2H), 7.48 (d, J=10.1 Hz, 1H), 7.4-7.4 (m, 2H), 7.28 (br d, J=7.9 Hz, 2H), 6.52 (d, J=8.8 Hz, 2H), 6.02 (br s, 1H), 3.81 (quin, J=5.7 Hz, 1H), 3.3-3.4 (m, 3H), 3.0-3.1 (m, 1H), 2.88 (br t, J=6.5 Hz, 2H), 1.8-2.0 (m, 3H), 1.6-1.6 (m, 1H), 1.04 (d, J=6.1 Hz, 3H,); LCMS: [M+H]⁺=417.5.

### Example 47: (R)-6-(2-amino-6-fluoro-5-(2-fluoro-4-(2-methylpyrrolidin-1-yl)phenyl)pyridin-3-yl)-3,4-dihydroisoquinolin-1(2H)-one (I-47)

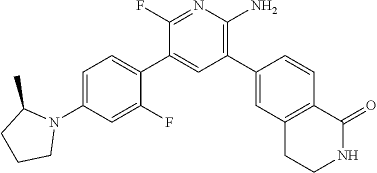 [SMILES: C[C@@H]1CCCN1c1ccc(-c2cc(-c3ccc4c(c3)CCNC4=O)c(N)nc2F)c(F)c1]

Step 1: (R)-3-chloro-6-fluoro-5-(2-fluoro-4-(2-methylpyrrolidin-1-yl)phenyl)pyridin-2-amine

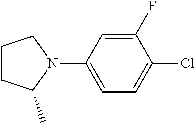

A 30 mL vial was charged with (R)-2-methylpyrrolidine hydrochloride (0.300 g, 2.467 mmol), 4-bromo-1-chloro-2-fluorobenzene (0.359 mL, 3.08 mmol), tris(dibenzylideneacetone)dipalladium (0) (0.068 g, 0.074 mmol), 4,5-bis(diphenylphosphino)-9,9-dimethylxanthene (0.128 g, 0.222 mmol) and Cs₂CO₃ (4.02 g, 12.3 mmol). The vial was sealed with a cap and septum and then the reaction vessel was evacuated and backfilled with nitrogen. Toluene (8 mL) was added, the reaction vessel was evacuated and backfilled with nitrogen an additional time, and was heated at 100° C. for 18 h. The reaction was concentrated onto Celite® and purified by flash chromatography (1-10% EtOAc/hexanes) to afford the product (268 mg, 51%). LCMS: [M+H]⁺=214.02.

Step 2: (R)-1-(3-fluoro-4-(4,4,5,5-tetramethyl-1,3,2-dioxaborolan-2-yl)phenyl)-2-methylpyrrolidine

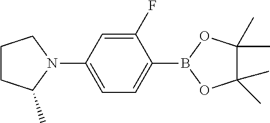 [SMILES: C[C@@H]1CCCN1c1ccc(B2OC(C)(C)C(C)(C)O2)c(F)c1]

A 30 ml vial was charged with (R)-1-(4-chloro-3-fluorophenyl)-2-methylpyrrolidine (0.268 g, 1.25 mmol), bis(pinacolato)diboron (0.478 g, 1.88 mmol), KOAc (0.246 g, 2.51 mmol) and XPhos Pd G2 (0.074 g, 0.094 mmol). The vial was evacuated and backfilled with nitrogen. 1,4-Dioxane (8 mL) was added and the vial was evacuated and backfilled an additional time. The reaction mixture was then heated to 110° C. in an aluminum block for 18 h. The reaction mixture was concentrated onto Celite® and purified by flash (0-8% EtOAc/hexanes) to afford the product (244 mg, 64%). LCMS: [M+H]⁺=306.06.

Step 3: (R)-3-chloro-6-fluoro-5-(2-fluoro-4-(2-methylpyrrolidin-1-yl)phenyl)pyridin-2-amine

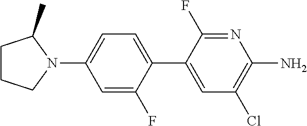 [SMILES: C[C@@H]1CCCN1c1ccc(-c2cc(Cl)c(N)nc2F)c(F)c1]

In a 30 mL vial with magnetic stir bar was charged 5-bromo-3-chloro-6-fluoropyridin-2-amine (0.055 g, 0.244 mmol), (R)-1-(3-fluoro-4-(4,4,5,5-tetramethyl-1,3,2-dioxaborolan-2-yl)phenyl)-2-methylpyrrolidine (0.082 g, 0.268 mmol) and [1,12-bis(diphenylphosphino)ferrocene]dichloropalladium(II), DCM complex (0.020 g, 0.024 mmol). The vial was sealed with a cap and septum and the reaction vessel was evacuated and backfilled with nitrogen. 1,4-Dioxane (2 mL) and aqueous Na₂CO₃ (0.366 mL of a 2 M solution, 0.732 mmol) were added and the reaction vessel was evacuated and backfilled with nitrogen an additional time. The reaction mixture was heated at 90° C. for 18 h in an aluminum block. The reaction mixture was concentrated onto Celite® and purified by flash (0-10% EtOAc/hexanes) to afford the product (244 mg, 64%). LCMS: [M+H]⁺=306.06.

Step 3: (R)-3-chloro-6-fluoro-5-(2-fluoro-4-(2-methylpyrrolidin-1-yl)phenyl)pyridin-2-amine

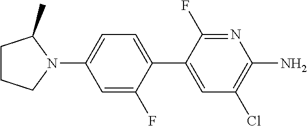 [SMILES: C[C@@H]1CCCN1c1ccc(-c2cc(Cl)c(N)nc2F)c(F)c1]

In a 30 mL vial with magnetic stir bar was charged 5-bromo-3-chloro-6-fluoropyridin-2-amine (0.055 g, 0.244 mmol), (R)-1-(3-fluoro-4-(4,4,5,5-tetramethyl-1,3,2-dioxaborolan-2-yl)phenyl)-2-methylpyrrolidine (0.082 g, 0.268 mmol) and [1,12-bis(diphenylphosphino)ferrocene]dichloropalladium(II), DCM complex (0.020 g, 0.024 mmol) The vial was sealed with a cap and septum and the reaction vessel was evacuated and backfilled with nitrogen. 1,4-Dioxane (2 mL) and aqueous Na₂CO₃ (2 M) (0.366 mL, 0.732 mmol) were added and the reaction vessel was evacuated and backfilled with nitrogen an additional time. The reaction mixture was heated at 90° C. for 18 h in an aluminum block. The reaction mixture was concentrated onto Celite® and purified by flash chromatography (0-10% EtOAc/hexanes) to afford the product (34 mg, 43%). LCMS: [M+H]⁺=323.88.

Step 4: (R)-6-(2-amino-6-fluoro-5-(2-fluoro-4-(2-methylpyrrolidin-1-yl)phenyl)pyridin-3-yl)-3,4-dihydroisoquinolin-1(2H)-one

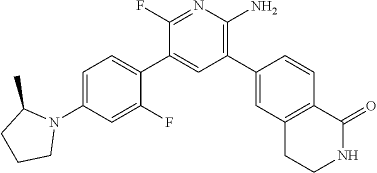 [SMILES: C[C@@H]1CCCN1c1ccc(-c2cc(-c3ccc4c(c3)CCNC4=O)c(N)nc2F)c(F)c1]

A procedure analogous to Example 43, Step 5 using 3,4-dihydro-1(2H)-isoquinolinone-6-boronic acid pinacol ester (0.033 g, 0.121 mmol), (R)-3-chloro-6-fluoro-5-(2-fluoro-4-(2-methylpyrrolidin-1-yl)phenyl)pyridin-2-amine (0.034 g, 0.105 mmol) and XPhos Pd G2 (8.26 mg, 10.50 μmol) and aqueous K₃PO₄ (0.242 mL of a 1.3 M solution, 0.315 mmol) afforded the title compound (3 mg, 6%) as a pale yellow powder. ¹H NMR (500 MHz, DMSO-d₆) δ 7.8-7.9 (m, 2H), 7.3-7.4 (m, 3H), 7.13 (t, J=8.8 Hz, 1H), 6.35 (dd, J=8.6, 2.3 Hz, 1H), 6.30 (dd, J=13.9, 2.2 Hz 1H), 6.13 (br d, J=2.4 Hz, 2H), 3.82 (quin, J=6.0 Hz, 1H), 3.3-3.4 (m, 4H), 3.0-3.1 (m, 1H), 2.87 (t, J=6.5 Hz, 2H), 1.9-2.0 (m, 4H), 1.6-1.6 (m, 1H), 1.04 (d, J=6.1 Hz, 3H); LCMS: [M+H]⁺=435.4.

### Example 48: (R)-6-(2-amino-6-fluoro-5-(4-(3-isopropyl-4-methylpiperazin-1-yl)phenyl)pyridin-3-yl)-3,4-dihydroisoquinolin-1(2H)-one (I-48)

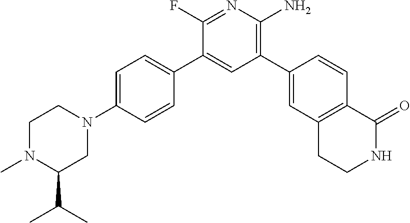 [SMILES: CC(C)[C@@H]1CN(c2ccc(-c3cc(-c4ccc5c(c4)CCNC5=O)c(N)nc3F)cc2)CCN1C]

Step 1: tert-butyl (R)-3-isopropyl-4-methylpiperazine-1-carboxylate

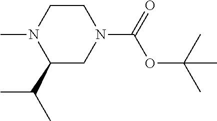 [SMILES: CC(C)[C@@H]1CN(C(=O)OC(C)(C)C)CCN1C]

To a solution of (R)-1-Boc-3-isopropyl-piperazine (1.0 g, 4.4 mmol) in 1:1 MeOH:THF (20 mL) was added an aqueous formaldehyde solution (37%, 0.5 mL, 6.6 mmol) followed by NaBH(OAc)₃ (1.4 g, 6.6 mmol). The reaction was stirred at RT for 18 h. The volatiles were removed in vaccuo and the residue was partitioned between aqueous KOH (1N) and DCM. The layers were separated and the aqueous layer was extracted with additional DCM (x2). The combined organic extracts were dried over MgSO₄ and concentrated to dryness to afford the product (1.06 g, quantitative yield). LCMS: [M+H]⁺=243.3.

Step 2: (R)-2-isopropyl-1-methylpiperazine

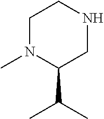

Trifluoroacetic acid (2.0 mL, 26 mmol) was added to a solution of tert-butyl (R)-3-isopropyl-4-methylpiperazine-1-carboxylate (0.50 g, 2.1 mmol) in DCM (10 mL) at RT. The reaction mixture was allowed to stir at RT for 18 h. The volatiles were removed under a stream of compressed air and the residue was dried under reduced pressure to afford the trifluoroacetic acid salt of the product (0.91 g, quantitative yield). LCMS: [M+H]⁺=143.4.

Step 3: (R)-4-(4-chlorophenyl)-2-isopropyl-1-methylpiperazine

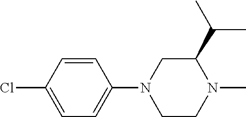 [SMILES: CC(C)[C@@H]1CN(c2ccc(Cl)cc2)CCN1C]

A 30 mL vial was charged with the trifluoroacetic acid salt of (R)-2-isopropyl-1-methylpiperazine (0.50 g, 1.4 mmol), 1-chloro-4-iodobenzene (0.40 g, 1.7 mmol), tris(dibenzylideneacetone)dipalladium (0) (0.037 g, 0.041 mmol), 4,5-bis(diphenylphosphino)-9,9-dimethylxanthene (0.070 g, 0.12 mmol) and Cs₂CO₃ (2.6 g, 8.1 mmol). The vial was sealed with a cap and septum and then the reaction vial was evacuated and backfilled with nitrogen. Toluene (6 mL) was added and the reaction vial was evacuated and backfilled with nitrogen an additional time. The reaction was heated conventionally at 100° C. for 18 h. After cooling to RT the reaction mixture was concentrated directly onto Celite® and purified by flash chromatography (0.5-9.5% DCM/MeOH+0.5% NH₄OH) to afford the product (0.18 g, 51%). LCMS: [M+H]⁺=253.3.

Step 4: (R)-2-isopropyl-1-methyl-4-(4-(4,4,5,5-tetramethyl-1,3,2-dioxaborolan-2-yl)phenyl)piperazine

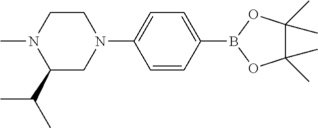

A 30 mL vial was charged with (R)-4-(4-chlorophenyl)-2-isopropyl-1-methylpiperazine (0.18 g, 0.69 mmol), bis(pinacolato)diboron (0.26 g, 1.0 mmol), KOAc (0.14 g, 1.4 mmol) and XPhos Pd G2 (0.041 g, 0.052 mmol). The vial was evacuated and backfilled with nitrogen. 1,4-Dioxane (6 mL) was added and the vial was evacuated and backfilled an additional time. The reaction mixture was then heated to 110° C. in an aluminum block for 18 h. The reaction mixture was concentrated onto Celite® and purified by flash chromatography (0.5-7.5% MeOH/DCM+0.5% NH₄OH) to afford the product (0.20 g, 83%). LCMS: [M+H]⁺=345.4.

Step 5: (R)-3-chloro-6-fluoro-5-(4-(3-isopropyl-4-methylpiperazin-1-yl)phenyl)pyridin-2-amine

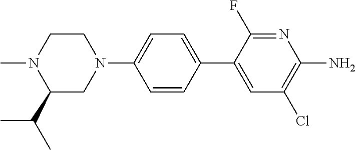 [SMILES: CC(C)[C@@H]1CN(c2ccc(-c3cc(Cl)c(N)nc3F)cc2)CCN1C]

A 30 mL was charged with 5-bromo-3-chloro-6-fluoropyridin-2-amine (0.040 g, 0.18 mmol), (R)-2-isopropyl-1-methyl-4-(4-(4,4,5,5-tetramethyl-1,3,2-dioxaborolan-2-yl)phenyl)piperazine (0.067 g, 0.20 mmol) and [1,12-bis(diphenylphosphino)ferrocene]dichloropalladium (II) DCM complex (0.014 g, 0.018 mmol) . The vial was sealed with a cap and septum and the reaction vessel was evacuated and backfilled with nitrogen. 1,4-Dioxane (2 mL) and 2 M aqueous Na₂CO₃ (0.27 mL, 0.53 mmol) were added and the reaction vessel was evacuated and backfilled with nitrogen an additional time. The reaction mixture was heated at 90° C. for 18 h in an aluminum block. The reaction mixture was concentrated onto Celite® and purified by flash chromatography (0.5-9.5% MeOH/DCM+0.5% NH₄OH) to afford the product (16 mg, 25%). LCMS: [M+H]⁺=363.2.

Step 6: (R)-6-(2-amino-6-fluoro-5-(4-(3-isopropyl-4-methylpiperazin-1-yl)phenyl)pyridin-3-yl)-3,4-dihydroisoquinolin-1(2H)-one

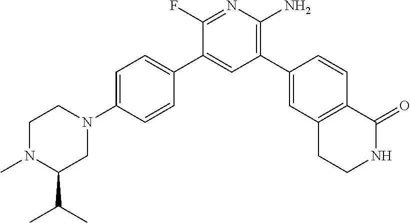 [SMILES: CC(C)[C@@H]1CN(c2ccc(-c3cc(-c4ccc5c(c4)CCNC5=O)c(N)nc3F)cc2)CCN1C]

A procedure analogous to Example 43, Step 5 using 3,4-dihydro-1(2H)-isoquinolinone-6-boronic acid pinacol ester (0.014 g, 0.051 mmol), (R)-3-chloro-6-fluoro-5-(4-(3-isopropyl-4-methylpiperazin-1-yl)phenyl)pyridin-2-amine (0.016 g, 0.044 mmol), XPhos Pd G2 (0.0035 mg, 0.0044 mmol) and aqueous K₃PO₄ (0.10 mL of a 1.3 M solution, 0.13 mmol) afforded the title compound (3 mg, 13%). ¹H NMR (500 MHz, DMSO-d₆) δ 7.8-7.9 (m, 2H), 7.51 (d, J=10.1 Hz, 1H), 7.4-7.4 (m, 2H), 7.33 (br d, J=7.8 Hz, 2H), 6.90 (d, J=8.9 Hz, 2H), 6.10 (br s, 2H), 3.50 (br d, J=11.7 Hz, 1H), 3.41 (br d, J=11.6 Hz, 1H), 3.3-3.4 (m, 3H), 2.88 (br t, J=6.4 Hz, 3H), 2.8-2.8 (m, 1H), 2.65 (dt, J=11.7, 2.8 Hz, 2H), 2.2-2.3 (m, 2H), 2.13 (s, 3H), 2.05 (dt, J=6.9, 4.2 Hz, 1H), 1.87 (td, J=10.5, 3.4 Hz, 1H), 0.92 (d, J=7.0 Hz, 3H), 0.81 (d, J=7.0 Hz, 3H); LCMS: [M+H]⁺=474.6.

### Example 49: (R)-6-(2-amino-6-fluoro-5-(2-fluoro-4-(3-isopropyl-4-methylpiperazin-1-yl)phenyl)pyridin-3-yl)-3,4-dihydroisoquinolin-1(2H)-one (I-49)

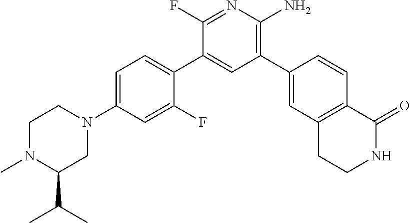 [SMILES: CC(C)[C@@H]1CN(c2ccc(-c3cc(-c4ccc5c(c4)CCNC5=O)c(N)nc3F)c(F)c2)CCN1C]

Step 1: (R)-4-(3-fluoro-4-(4,4,5,5-tetramethyl-1,3,2-dioxaborolan-2-yl)phenyl)-2-isopropyl-1-methylpiperazine

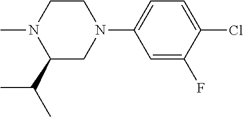 [SMILES: CC(C)[C@@H]1CN(c2ccc(Cl)c(F)c2)CCN1C]

A procedure analogous to Example 48, Step 3 using (R)-2-isopropyl-1-methylpiperazine-2TFA (0.500 g, 1.35 mmol), 4-bromo-1-chloro-2-fluorobenzene (0.196 mL, 1.69 mmol), tris(dibenzylideneacetone)dipalladium (0) (0.037 g, 0.041 mmol), 4,5-bis(diphenylphosphino)-9,9-dimethylxanthene (0.070 g, 0.122 mmol) and Cs₂CO₃ (2.64 g, 8.10 mmol) afforded the product (167 mg, 46%). LCMS: [M+H]⁺=271.09.

Step 2: (R)-4-(3-fluoro-4-(4,4,5,5-tetramethyl-1,3,2-dioxaborolan-2-yl)phenyl)-2-isopropyl-1-methylpiperazine

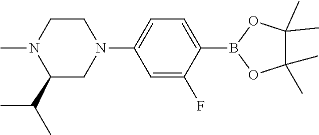 [SMILES: CC(C)[C@@H]1CN(c2ccc(B3OC(C)(C)C(C)(C)O3)c(F)c2)CCN1C]

A procedure analogous to Example 48, Step 4 using (R)-4-(4-chloro-3-fluorophenyl)-2-isopropyl-1-methylpiperazine (0.167 g, 0.617 mmol), bis(pinacolato)diboron (0.235 g, 0.925 mmol), KOAc (0.121 g, 1.23 mmol) and XPhos Pd G2 (0.036 g, 0.046 mmol) afforded the product (263 mg, quantitative yield). LCMS [M+H]⁺=363.30 Step 3: (R)-6-(2-amino-6-fluoro-5-(2-fluoro-4-(3-isopropyl-4-methylpiperazin-1-yl)phenyl)pyridin-3-yl)-3,4-dihydroisoquinolin-1(2H)-one

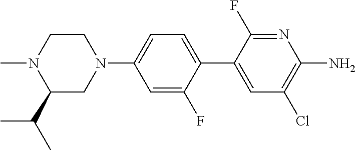 [SMILES: CC(C)[C@@H]1CN(c2ccc(-c3cc(Cl)c(N)nc3F)c(F)c2)CCN1C]

A procedure analogous to Example 4, Step 5 using 5-bromo-3-chloro-6-fluoropyridin-2-amine (0.037 g, 0.164 mmol), (R)-4-(3-fluoro-4-(4,4,5,5-tetramethyl-1,3,2-dioxaborolan-2-yl)phenyl)-2-isopropyl-1-methylpiperazine (0.065 g, 0.181 mmol) and [1,12-bis(diphenylphosphino)ferrocene]dichloropalladium(II), DCM complex (0.013 g, 0.016 mmol) afforded the product (14 mg, 22%). LCMS: [M+H]⁺=381.20.

Step 4: (R)-6-(2-amino-6-fluoro-5-(2-fluoro-4-(3-isopropyl-4-methylpiperazin-1-yl)phenyl)pyridin-3-yl)-3,4-dihydroisoquinolin-1(2H)-one

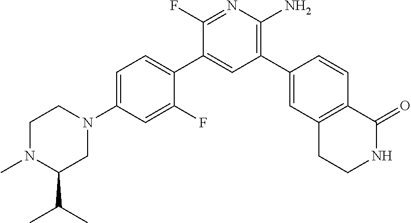 [SMILES: CC(C)[C@@H]1CN(c2ccc(-c3cc(-c4ccc5c(c4)CCNC5=O)c(N)nc3F)c(F)c2)CCN1C]

A procedure analogous to Example 43, Step 5 using 3,4-dihydro-1(2H)-isoquinolinone-6-boronic acid pinacol ester (0.012 g, 0.042 mmol), (R)-3-chloro-6-fluoro-5-(2-fluoro-4-(3-isopropyl-4-methylpiperazin-1-yl)phenyl)pyridin-2-amine (0.014 g, 0.037 mmol) and XPhos Pd G2 (2.89 mg, 3.68 μmol) and aqueous K₃PO₄ (0.085 mL of a 1.3 M solution, 0.110 mmol) afforded the title compound (5 mg, 26%) as a pale yellow powder. ¹H NMR (500 MHz, DMSO-d₆) δ 7.8-7.9 (m, 2H), 7.3-7.4 (m, 3H), 7.20 (t, J=8.7 Hz, 1H), 6.74 (br d, J=11.5 Hz, 2H), 6.19 (s, 2H), 3.55 (br d, J=11.9 Hz, 1H), 3.43 (br d, J=11.9 Hz, 1H), 3.33 (dt, J=6.5, 2.9 Hz, 2H), 2.87 (br t, J=6.5 Hz, 2H), 2.8-2.8 (m, 1H), 2.69 (dt, J=11.8, 2.9 Hz, 1H), 2.21 (dt, J=11.6, 3.1 Hz, 1H), 2.12 (s, 3H), 2.05 (qd, J=11.1, 6.9 Hz, 1H,), 1.85 (td, J=10.7, 3.4 Hz, 1H), 0.92 (d, J=7.0 Hz, 3H), 0.81 (d, J=7.0 Hz, 3H); LCMS [M+H]⁺=492.4.

### Example 50: (S)-6-(2-amino-6-fluoro-5-(4-(3-isopropyl-4-methylpiperazin-1-yl)phenyl)pyridin-3-yl)-3,4-dihydroisoquinolin-1(2H)-one (I-50)

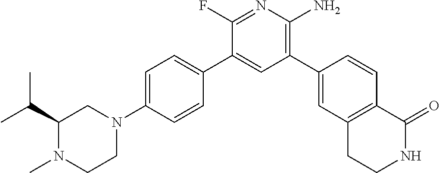 [SMILES: CC(C)[C@H]1CN(c2ccc(-c3cc(-c4ccc5c(c4)CCNC5=O)c(N)nc3F)cc2)CCN1C]

Step 1: tert-butyl (S)-3-isopropyl-4-methylpiperazine-1-carboxylate

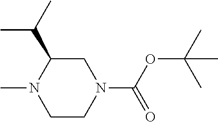 [SMILES: CC(C)[C@H]1CN(C(=O)OC(C)(C)C)CCN1C]

A procedure analogous to Example 48, Step 1 using (S)-1-Boc-3-isopropyl-piperazine (1.0 g, 4.38 mmol) (1.0 g, 4.38 mmol) in MeOH/THF (10 mL each), formaldehyde solution, 37% wt in water (0.489 ml, 6.57 mmol) and NaBH(OAC)₃ (1.39 g, 6.57 mmol) afforded the product (1.09 g, quantitative yield) as a clear oil. LCMS: [M+H]=243.42.

Step 2: (S)-2-isopropyl-1-methylpiperazine 2TFA

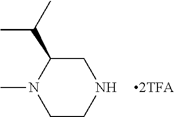

A procedure analogous to Example 48, Step 2 using TFA (1.97 mL, 25.8 mmol), tert-butyl (S)-3-isopropyl-4-methylpiperazine-1-carboxylate (0.50 g, 2.06 mmol) in CH₂Cl₂ (10 mL) at RT afforded the product (1.05 g, quantitative yield). LCMS: [M+H]⁺=143.42.

Step 3: (S)-4-(4-chlorophenyl)-2-isopropyl-1-methylpiperazine

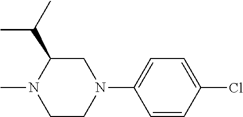 [SMILES: CC(C)[C@H]1CN(c2ccc(Cl)cc2)CCN1C]

A procedure analogous to Example 48, Step 3 using (S)-2-isopropyl-1-methylpiperazine-2TFA (0.500 g, 1.35 mmol), 1-chloro-4-iodobenzene (0.402 g, 1.69 mmol), tris(dibenzylideneacetone)dipalladium (0) (0.037 g, 0.041 mmol), 4,5-bis(diphenylphosphino)-9,9-dimethylxanthene (0.070 g, 0.122 mmol) and Cs₂CO₃ (2.64 g, 8.10 mmol) afforded the product (150 mg, 44%). LCMS: [M+H]⁺=253.27.

Step 4: ((S)-2-isopropyl-1-methyl-4-(4-(4,4,5,5-tetramethyl-1,3,2-dioxaborolan-2-yl)phenyl)piperazine

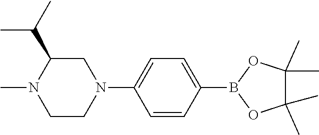

A procedure analogous to Example 48, Step 4 using (S)-4-(4-chlorophenyl)-2-isopropyl-1-methylpiperazine (0.150 g, 0.593 mmol), bis(pinacolato)diboron (0.226 g, 0.890 mmol), KOAc (0.116 g, 1.19 mmol) and XPhos Pd G2 (0.035 g, 0.045 mmol) afforded the product (158 mg, 77%). LCMS: [M+H]⁺=345.32

Step 5: (S)-6-(2-amino-6-fluoro-5-(4-(3-isopropyl-4-methylpiperazin-1-yl)phenyl)pyridin-3-yl)-3,4-dihydroisoquinolin-1(2H)-one

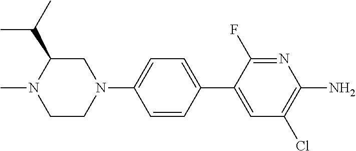 [SMILES: CC(C)[C@H]1CN(c2ccc(-c3cc(Cl)c(N)nc3F)cc2)CCN1C]

A procedure analogous to Example 48, Step 5 using 5-bromo-3-chloro-6-fluoropyridin-2-amine (0.045 g, 0.200 mmol), (S)-2-isopropyl-1-methyl-4-(4-(4,4,5,5-tetramethyl-1,3,2-dioxaborolan-2-yl)phenyl)piperazine (0.076 g, 0.220 mmol) and [1,12-bis(diphenylphosphino)ferrocene]dichloropalladium(II), DCM complex (0.016 g, 0.020 mmol) afforded the product (29 mg, 40%). LCMS: [M+H]⁺=363.30.

Step 6: (S)-6-(2-amino-6-fluoro-5-(4-(3-isopropyl-4-methylpiperazin-1-yl)phenyl)pyridin-3-yl)-3,4-dihydroisoquinolin-1(2H)-one

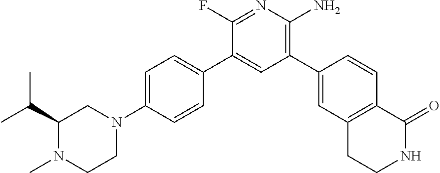 [SMILES: CC(C)[C@H]1CN(c2ccc(-c3cc(-c4ccc5c(c4)CCNC5=O)c(N)nc3F)cc2)CCN1C]

A procedure analogous to Example 43, Step 5 using 3,4-dihydro-1(2H)-isoquinolinone-6-boronic acid pinacol ester (0.025 g, 0.092 mmol), (S)-3-chloro-6-fluoro-5-(4-(3-isopropyl-4-methylpiperazin-1-yl)phenyl)pyridin-2-amine (0.029 g, 0.080 mmol) and XPhos Pd G2 (6.29 mg, 7.99 μmol) and aqueous K₃PO₄ (0.184 mL of a 1.3 M solution, 0.240 mmol) afforded the title compound (8 mg, 20%) as a yellow powder. ¹H NMR (500 MHz, DMSO-d₆) δ 7.8-7.9 (m, 2H), 7.51 (d, J=10.1 Hz, 1H), 7.4-7.4 (m, 2H), 7.33 (br d, J=7.8 Hz, 2H), 6.90 (d, J=8.9 Hz, 2H), 6.10 (br s, 2H), 3.5-3.5 (m, 1H), 3.41 (br d, J=11.7 Hz, 1H), 3.34 (dt, J=6.4, 2.9 Hz, 2H), 2.88 (br t, J=6.5 Hz, 2H), 2.8-2.8 (m, 1H), 2.65 (dt, J=11.7, 2.9 Hz, 1H), 2.23 (dt, J=11.5, 3.0 Hz, 1H), 2.12 (s, 3H), 2.05 (dt, J=6.9, 4.3 Hz, 1H), 1.87 (td, J=10.7, 3.4 Hz, 1H), 0.92 (d, J=7.0 Hz, 3H), 0.81 (d, J=7.0 Hz, 3H); LCMS: [M+H]⁺=474.5.

### Example 51: (S)-6-(2-amino-6-fluoro-5-(2-fluoro-4-(3-isopropyl-4-methylpiperazin-1-yl)phenyl)pyridin-3-yl)-3,4-dihydroisoquinolin-1(2H)-one (I-51)

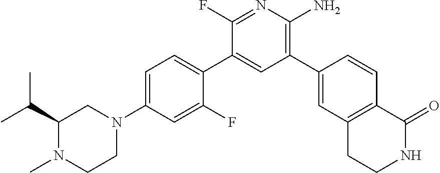 [SMILES: CC(C)[C@H]1CN(c2ccc(-c3cc(-c4ccc5c(c4)CCNC5=O)c(N)nc3F)c(F)c2)CCN1C]

Step 1: (S)-4-(3-fluoro-4-(4,4,5,5-tetramethyl-1,3,2-dioxaborolan-2-yl)phenyl)-2-isopropyl-1-methylpiperazine

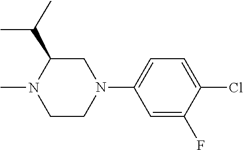 [SMILES: CC(C)[C@H]1CN(c2ccc(Cl)c(F)c2)CCN1C]

A procedure analogous to Example 48, Step 3 using (S)-2-isopropyl-1-methylpiperazine-2TFA (0.500 g, 1.35 mmol), 4-bromo-1-chloro-2-fluorobenzene (0.196 mL, 1.69 mmol), tris(dibenzylideneacetone)dipalladium (0) (0.037 g, 0.041 mmol), 4,5-bis(diphenylphosphino)-9,9-dimethylxanthene (0.070 g, 0.122 mmol) and Cs₂CO₃ (2.64 g, 8.10 mmol) afforded the product (178 mg, 49%). LCMS: [M+H]⁺=271.01.

Step 2: (S)-4-(3-fluoro-4-(4,4,5,5-tetramethyl-1,3,2-dioxaborolan-2-yl)phenyl)-2-isopropyl-1-methylpiperazine

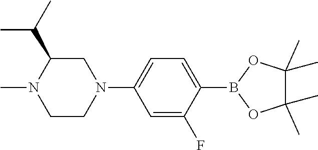

A procedure analogous to Example 43, Step 4 using (S)-4-(4-chloro-3-fluorophenyl)-2-isopropyl-1-methylpiperazine (0.178 g, 0.657 mmol), bis(pinacolato)diboron (0.250 g, 0.986 mmol), KOAc (0.129 g, 1.315 mmol) and XPhos Pd G2 (0.039 g, 0.049 mmol) afforded the product (252 mg, quantitative yield). LCMS: [M+H]⁺=363.22.

Step 3: (S)-3-chloro-6-fluoro-5-(2-fluoro-4-(3-isopropyl-4-methylpiperazin-1-yl)phenyl)pyridin-2-amine

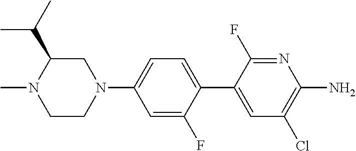 [SMILES: CC(C)[C@H]1CN(c2ccc(-c3cc(Cl)c(N)nc3F)c(F)c2)CCN1C]

A procedure analogous to Example 43, Step 5 using 5-bromo-3-chloro-6-fluoropyridin-2-amine (0.035 g, 0.155 mmol), (S)-4-(3-fluoro-4-(4,4,5,5-tetramethyl-1,3,2-dioxaborolan-2-yl)phenyl)-2-isopropyl-1-methylpiperazine (0.062 g, 0.171 mmol) and [1,12-bis(diphenylphosphino)ferrocene]dichloropalladium(II), DCM complex (0.013 g, 0.016 mmol) afforded the product (17 mg, 29%). LCMS: [M+H]⁺=381.28.

Step 4: (S)-6-(2-amino-6-fluoro-5-(2-fluoro-4-(3-isopropyl-4-methylpiperazin-1-yl)phenyl)pyridin-3-yl)-3,4-dihydroisoquinolin-1(2H)-one

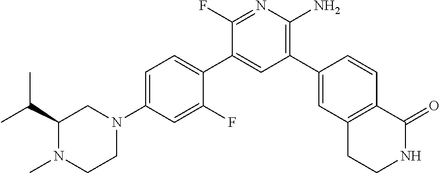 [SMILES: CC(C)[C@H]1CN(c2ccc(-c3cc(-c4ccc5c(c4)CCNC5=O)c(N)nc3F)c(F)c2)CCN1C]

A procedure analogous to Example 43, Step 5 using 3,4-dihydro-1(2H)-isoquinolinone-6-boronic acid pinacol ester (0.014 g, 0.051 mmol), (S)-3-chloro-6-fluoro-5-(2-fluoro-4-(3-isopropyl-4-methylpiperazin-1-yl)phenyl)pyridin-2-amine (0.017 g, 0.045 mmol), XPhos Pd G2 (3.51 mg, 4.46 μmol) and aqueous K₃PO₄ (0.103 mL of a 1.3 M solution, 0.134 mmol) afforded the title compound (4 mg, 17%) as a yellow powder. ¹H NMR (500 MHz, DMSO-d₆) δ 7.8-7.9 (m, 2H), 7.3-7.4 (m, 3H), 7.20 (t, J=8.7 Hz, 1H), 6.74 (br d, J=11.4 Hz, 2H), 6.19 (s, 2H), 3.55 (br d, J=11.7 Hz, 1H), 3.43 (br d, J=11.7 Hz, 1H), 3.33 (td, J=6.3, 3.2 Hz, 2H), 2.87 (br t, J=6.4 Hz, 2H), 2.78 (br d, J=11.4 Hz, 1H), 2.69 (dt, J=11.8, 2.6 Hz, 1H), 2.21 (dt, J=11.6, 3.0 Hz, 1H), 2.12 (s, 3H), 2.05 (qd, J=11.1, 6.8 Hz, 1H), 1.85 (td, J=10.5, 3.2 Hz, 1H), 0.92 (d, J=6.8 Hz, 3H), 0.81 (d, J=6.8 Hz, 3H); LCMS: [M+H]⁺=492.5.

### Example 52: 6-(2-amino-5-(4-(1-ethylpiperidin-3-yl)phenyl)-6-fluoropyridin-3-yl)-3,4-dihydroisoquinolin-1(2H)-one (I-52)

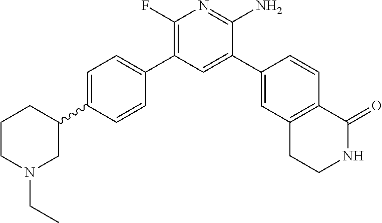 [SMILES: CCN1CCCC(c2ccc(-c3cc(-c4ccc5c(c4)CCNC5=O)c(N)nc3F)cc2)C1]

Step 1: 3-(4-bromophenyl)-1-ethylpiperidine

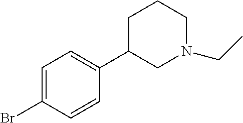

To a slurry of 3-(4-bromo-phenyl)-piperidine hydrochloride (300 mg, 1.09 mmol) in DMF (5 mL) at RT was added K₂CO₃ (600 mg, 4.34 mmol) and bromoethane (0.121 ml, 1.63 mmol) and the mixture was stirred at RT for 1.5 h. The reaction mixture was concentrated to dryness, taken up in DCM/water, and the organic phase was separated. The aqueous phase was extracted with DCM (x 2), the combined organic layers were washed (x 3) with water and with brine, dried over Na₂SO₄ and concentrated to yield the product. LCMS: [M+H]⁺=269.98.

Step 2: 6-(2-amino-5-(4-(1-ethylpiperidin-3-yl) phenyl)-6-fluoropyridin-3-yl)-3,4-dihydroisoquinolin-1(2H)-one

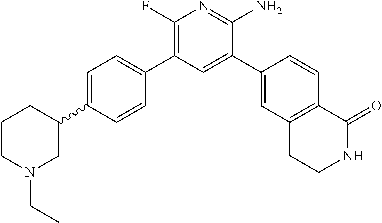 [SMILES: CCN1CCCC(c2ccc(-c3cc(-c4ccc5c(c4)CCNC5=O)c(N)nc3F)cc2)C1]

A mixture of 6-(2-amino-6-fluoro-5-(4,4,5,5-tetramethyl-1,3,2-dioxaborolan-2-yl)pyridin-3-yl)-3,4-dihydroisoquinolin-1(2H)-one (138 mg, 0.287 mmol) (3.5 mL of the stock solution of 40 mg/mL), Cs₂CO₃ (200 mg, 0.615 mmol), 3-(4-bromophenyl)-1-ethylpiperidine (55 mg, 0.205 mmol), [1,1′-bis(diphenylphosphino)ferrocene]dichloropalladium(II) (22.51 mg, 0.031 mmol) and water (1 mL) in a microwave vial was flushed with Argon and heated in a microwave reactor at 90° C. for 1 h. The reaction mixture was partitioned between brine and EtOAc, the organic phase was separated and the aqueous phase was extracted with EtOAc. The combined organic layers were dried over Na₂SO₄ and concentrated onto Celite®. Purification by reverse phase flash chromatography on a RediSep column (5.5 G), eluting with water containing 0-35% acetonitrile, afforded the title compound was isolated as a beige solid (54 mg, 56%). ¹H NMR (500 MHz, CD₃OD) δ 8.0-8.1 (m, 1H), 7.6-7.7 (m, 1H), 7.4-7.5 (m, 4H), 7.3-7.3 (m, 2H), 3.5-3.6 (m, 2H), 3.1-3.2 (m, 2H), 3.0-3.1 (m, 2H), 2.8-2.9 (m, 1H), 2.57 (q, J=6.8 Hz, 2H), 2.1-2.2 (m, 2H), 1.9-2.0 (m, 1H), 1.9-1.9 (m, 1H), 1.7-1.8 (m, 1H), 1.5-1.6 (m, 1H), 1.15 (t, J=7.2 Hz, 3H); LCMS: [M+H]⁺=445.16.

### Example 53: 6-(2-amino-5-(4-(1-ethylpiperidin-4-yl)phenyl)-6-fluoropyridin-3-yl)-3,4-dihydroisoquinolin-1(2H)-one (I-53)

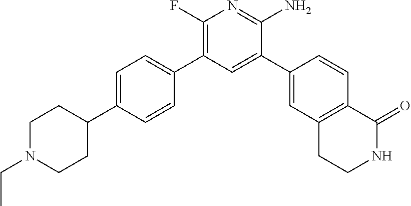 [SMILES: CCN1CCC(c2ccc(-c3cc(-c4ccc5c(c4)CCNC5=O)c(N)nc3F)cc2)CC1]

Step 1: 4-(4-bromophenyl)-1-ethylpiperidine

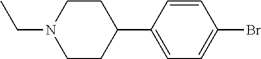

To a slurry of 4-(4-bromophenyl)piperidine (520 mg, 2.165 mmol) in DMF (6 mL) at RT, was added K₂CO₃ (898 mg, 6.50 mmol) followed by bromoethane (0.241 mL, 3.25 mmol), and the reaction mixture was stirred at RT for 1.5 h. The mixture was concentrated to dryness and the residue was partitioned between CH₂Cl₂ and water. The layers were separated and the aqueous layer was extracted with CH₂Cl₂ (x2). The combined organic phase was washed with water, brine, dried over Na₂SO₄ and concentrated to yield the title compound as a light yellow oil (274 mg, 47%). As the purity was good, it was taken to the next step without any purification. LCMS: [M+H]⁺=268.39.

Step 2: 6-(2-amino-5-(4-(1-ethylpiperidin-4-yl)phenyl)-6-fluoropyridin-3-yl)-3,4-dihydroisoquinolin-1(2H)-one

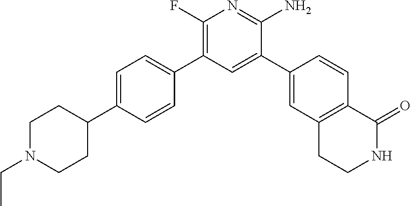 [SMILES: CCN1CCC(c2ccc(-c3cc(-c4ccc5c(c4)CCNC5=O)c(N)nc3F)cc2)CC1]

A procedure similar to Example 52, Step 2 using 6-(2-amino-6-fluoro-5-(4,4,5,5-tetramethyl-1,3,2-dioxaborolan-2-yl)pyridin-3-yl)-3,4-dihydroisoquinolin-1(2H)-one (147 mg, 0.308 mmol) and 4-(4-bromophenyl)-1-ethylpiperidine (55 mg, 0.205 mmol) afforded the title compound as a beige solid (30 mg, 31%). ¹H NMR (500 MHz, DMSO-d₆) δ 7.9-8.0 (m, 2H), 7.6-7.7 (m, 1H), 7.5-7.5 (m, 4H), 7.3-7.3 (m, 2H), 6.3-6.3 (m, 2H), 3.4-3.4 (m, 2H), 3.2-3.3 (m, 1H), 3.1-3.2 (m, 2H), 2.9-3.0 (m, 2H), 2.4-2.5 (m, 4H), 1.8-1.9 (m, 2H), 1.7-1.8 (m, 2H), 1.1-1.1 (m, 3H). LCMS: [M+H]⁺=445.41.

### Example 54: 6-(2-amino-6-fluoro-5-(4-((1-(methylsulfonyl)piperidin-4-yl)oxy)phenyl)pyridin-3-yl)-3,4-dihydroisoquinolin-1(2H)-one (I-54)

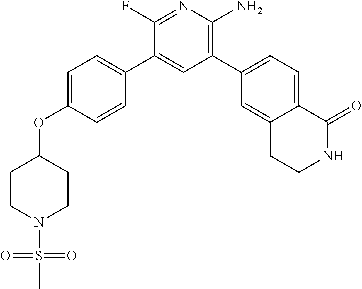 [SMILES: CS(=O)(=O)N1CCC(Oc2ccc(-c3cc(-c4ccc5c(c4)CCNC5=O)c(N)nc3F)cc2)CC1]

To a suspension of 6-(2-amino-6-fluoro-5-(4-(piperidin-4-yloxy)phenyl)pyridin-3-yl)-3,4-dihydroisoquinolin-1(2H)-one (24.9 mg, 0.058 mmol) and N,N-diisopropylethylamine (0.030 ml, 0.173 mmol) in CH₂Cl₂ (3 mL) was added methanesulfonyl chloride (8.91 μl, 0.115 mmol) at RT. The reaction mixture was stirred for 1 h at 23° C. The reaction mixture was dry loaded and purified using flash chromatography (0-20%, MeOH/DCM) to afford the title compound (24.1 mg, 78%) as a white powder. ¹H NMR (500 MHz, DMSO-d₆) δ 7.94 (br s, 1H), 7.91 (d, J=7.9 Hz, 1H), 7.61 (d, J=10.1 Hz, 1H), 7.52-7.44 (m, 4H), 7.04 (d, J=8.8 Hz, 2H), 6.24 (s, 2H), 4.65-4.57 (m, 1H), 3.41 (br d, J=2.4 Hz, 2H), 3.39-3.33 (m, 2H), 3.18-3.11 (m, 2H), 2.99-2.94 (m, 2H), 2.91 (s, 3H), 2.01 (br s, 2H), 1.77 (br s, 2H); LCMS: [M+H]⁺=511.28.

### Example 55: 6-(2-amino-6-fluoro-5-(1-(tetrahydro-2H-pyran-4-yl)-1H-pyrazol-4-yl)pyridin-3-yl)-3,4-dihydroisoquinolin-1(2H)-one (I-55)

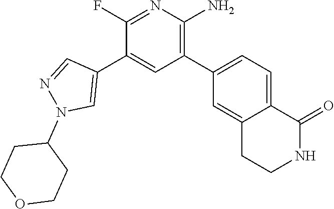 [SMILES: Nc1nc(F)c(-c2cnn(C3CCOCC3)c2)cc1-c1ccc2c(c1)CCNC2=O]

Step 1: 3-chloro-6-fluoro-5-(1-(tetrahydro-2H-pyran-4-yl)-1H-pyrazol-4-yl)pyridin-2-amine

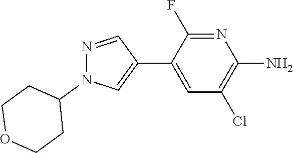 [SMILES: Nc1nc(F)c(-c2cnn(C3CCOCC3)c2)cc1Cl]

A procedure analogous to Example 47, Step 3 using 5-bromo-3-chloro-6-fluoropyridin-2-amine (150 mg, 0.665 mmol), PdCl₂dppf (48.7 mg, 0.067 mmol), 1-(tetrahydro-pyran-4-yl)-1H-pyrazole-4-boronic acid pinacol ester (185 mg, 0.665 mmol), Cs₂CO₃ (650 mg, 2.00 mmol), H₂O (5 mL) and DME (10 mL) the product as a dark brown solid (311 mg, 95% yield based on 60% purity). LCMS: [M+H]⁺=297.11.

Step 2: 6-(2-Amino-6-fluoro-5-(1-(tetrahydro-2H-pyran-4-yl)-1H-pyrazol-4-yl)pyridin-3-yl)-3,4-dihydroisoquinolin-1(2H)-one

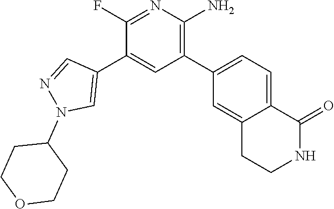 [SMILES: Nc1nc(F)c(-c2cnn(C3CCOCC3)c2)cc1-c1ccc2c(c1)CCNC2=O]

A procedure analogous to Example 1, General Method A using 3-chloro-6-fluoro-5-(1-(tetrahydro-2H-pyran-4-yl)-1H-pyrazol-4-yl)pyridin-2-amine (155 mg, 0.313 mmol, purity of 60%), K₃PO₄ (200 mg, 0.940 mmol), 3,4-dihydro-1(2H)-isoquinolinone-6-boronic acid pinacol ester (111 mg, 0.407 mmol), H₂O (6 mL), MeCN (9 mL) and XPhos Pd G2 (24.7 mg, 0.031 mmol) afforded the title compound as an off white solid (9 mg, 7%). ¹H NMR (500 MHz, CDC₃) δ 8.11 (d, J=8.0 Hz, 1H), 7.66-7.74 (m, 2H), 7.57 (d, J=9.3 Hz, 1H), 7.38-7.41 (m, 1H), 7.26 (s, 1H), 5.96-6.09 (m, 1H), 4.53-4.64 (m, 2H), 4.24-4.38 (m, 1H), 4.01-4.10 (m, 2H), 3.41-3.61 (m, 4H), 2.96-3.05 (m, 2H), 2.01-2.12 (m, 4H). LCMS: [M+H]⁺=408.28.

### Example 56: tert-butyl4-(4-(6-amino-2-fluoro-5-(1-oxo-1,2,3,4-tetrahydroisoquinolin-6-yl)pyridin-3-yl)phenyl)-3,6-dihydropyridine-1(2H)-carboxylate (1-56)

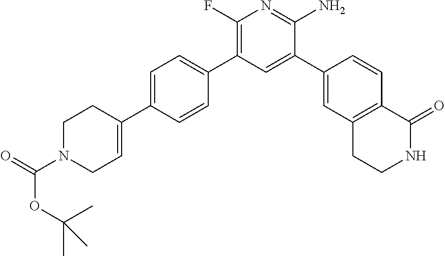 [SMILES: CC(C)(C)OC(=O)N1CC=C(c2ccc(-c3cc(-c4ccc5c(c4)CCNC5=O)c(N)nc3F)cc2)CC1]

A procedure analogous to Example 43, Step 5 using 6-(2-amino-5-bromo-6-fluoropyridin-3-yl)-3,4-dihydroisoquinolin-1(2H)-one (300 mg, 0.892 mmol) and (4-(1-(tert-butoxycarbonyl)-1,2,3,6-tetrahydropyridin-4-yl)phenyl)boronic acid pinacol ester (447 mg, 1.16 mmol) afforded the title compound as a beige solid (353 mg, 73%). ¹H NMR (500 MHz, DMSO-d₆) δ 7.9-8.0 (m, 1H), 7.9-7.9 (m, 1H), 7.6-7.7 (m, 1H), 7.5-7.6 (m, 2H), 7.4-7.5 (m, 4H), 6.3-6.4 (m, 2H), 6.2-6.3 (m, 1H), 4.0-4.1 (m, 2H), 3.5-3.6 (m, 2H), 3.4-3.4 (m, 2H), 3.3-3.4 (m, 2H), 2.9-3.0 (m, 2H), 1.4-1.5 (m, 9H); LCMS: [M+H]⁺=515.49.

### Example 57 6-(2-amino-6-fluoro-5-(4-(1,2,3,6-tetrahydropyridin-4-yl)phenyl)pyridin-3-yl)-3,4-dihydroisoquinolin-1(2H)-one 2,2,2-trifluoroacetate (1-57)

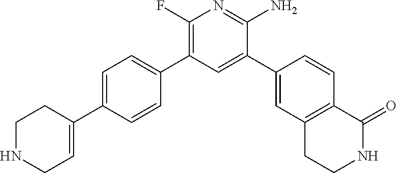 [SMILES: Nc1nc(F)c(-c2ccc(C3=CCNCC3)cc2)cc1-c1ccc2c(c1)CCNC2=O]

Tert-butyl 4-(4-(6-amino-2-fluoro-5-(1-oxo-1,2,3,4-tetrahydroisoquinolin-6-yl)pyridin-3-yl)phenyl)-3,6-dihydropyridine-1(2H)-carboxylate (141 mg, 0.215 mmol, prepared in Example 56) was stirred at RT with CH₂Cl₂ (2 mL) and TFA (0.5 mL) for 1 h. The reaction mixture was concentrated onto Celite® and purification by reverse phase chromatography (C18; 0-10% CH₃CN/water) afforded the title compound as an off white solid (68 mg, 57%). ¹H NMR (500 MHz, DMSO-d₆) δ 8.93-8.70 (m, 2H), 8.01-7.94 (m, 1H), 7.94-7.89 (m, 1H), 7.73-7.65 (m, 1H), 7.63-7.57 (m, 2H), 7.57-7.51 (m, 2H), 7.50-7.45 (m, 2H), 6.44-6.31 (m, 2H), 6.31-6.23 (m, 1H), 3.84-3.76 (m, 2H), 3.45-3.40 (m, 2H), 3.39-3.36 (m, 2H), 3.00-2.93 (m, 2H), 2.75-2.68 (m, 2H). LCMS: [M+H]⁺=415.45.

### Example 58: 6-(2-amino-6-fluoro-5-(4-methyl-3,4-dihydro-2H-benzo[b][1,4]oxazin-7-yl)pyridin-3-yl)-3,4-dihydroisoquinolin-1(2H)-one (I-58)

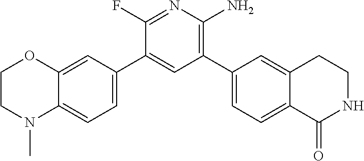 [SMILES: CN1CCOc2cc(-c3cc(-c4ccc5c(c4)CCNC5=O)c(N)nc3F)ccc21]

A procedure similar to Example 43, Step 5 using 6-(2-amino-5-bromo-6-fluoropyridin-3-yl)-3,4-dihydroisoquinolin-1(2H)-one (114 mg, 0.339 mmol) and 4-methyl-7-(4,4,5,5-tetramethyl-1,3,2-dioxaborolan-2-yl)-3,4-dihydro-2H-1,4-benzoxazine (112 mg, 0.407 mmol) afforded the title compound (95 mg, 69%) as a tan solid. ¹H NMR (500 MHz, DMSO-d₆) δ 7.93 (br s, 1H), 7.90 (d, J=7.9 Hz, 1H), 7.54 (d, J=10.1 Hz, 1H), 7.46 (d, J=7.9 Hz, 1H), 7.44 (s, 1H), 6.97 (br d, J=8.4 Hz, 1H), 6.87 (s, 1H), 6.72 (d, J=8.4 Hz, 1H), 6.15 (s, 2H), 4.27-4.20 (m, 2H), 3.40 (td, J=6.5, 2.7 Hz, 3H), 3.27-3.21 (m, 3H), 2.99-2.91 (m, 3H), 2.84 (s, 4H); LCMS: [M+H]⁺=405.13.

### Example 59: 6-(2-amino-6-fluoro-5-(4-(pyrrolidin-1-yl)phenyl)pyridin-3-yl)-3,4-dihydroisoquinolin-1(2H)-one (I-59)

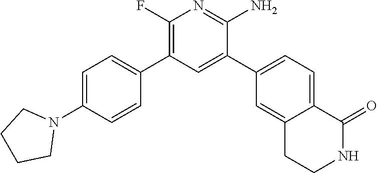 [SMILES: Nc1nc(F)c(-c2ccc(N3CCCC3)cc2)cc1-c1ccc2c(c1)CCNC2=O]

A procedure analogous to Example 43, Step 5 using 6-(2-amino-5-bromo-6-fluoropyridin-3-yl)-3,4-dihydroisoquinolin-1(2H)-one (120 mg, 0.357 mmol) and 1-[4-(4,4,5,5-tetramethyl-1,3,2-dioxaborolan-2-yl)phenyl]pyrrolidine (117 mg, 0.428 mmol) afforded the title compound (11 mg, 8%) as a brown solid. ¹H NMR (500 MHz, DMSO-d₆) δ 7.94 (br s, 1H), 7.90 (d, J=7.8 Hz, 1H), 7.55 (d, J=10.1 Hz, 1H), 7.46 (br d, J=7.9 Hz, 1H), 7.44 (s, 1H), 7.35 (br d, J=8.1 Hz, 2H), 6.57 (d, J=8.7 Hz, 2H), 6.10 (s, 2H), 3.43-3.38 (m, 2H), 3.23 (br t, J=6.2 Hz, 4H), 2.95 (br t, J=6.4 Hz, 2H), 1.95 (br t, J=6.3 Hz, 4H); LCMS: [M+H]⁺=403.10.

### Example 60: 6-(2-amino-6-fluoro-5-(4-(piperidin-1-yl)phenyl)pyridin-3-yl)-3,4-dihydroisoquinolin-1(2H)-one (I-60)

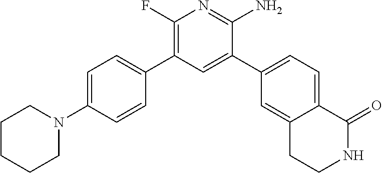 [SMILES: Nc1nc(F)c(-c2ccc(N3CCCCC3)cc2)cc1-c1ccc2c(c1)CCNC2=O]

A procedure analogous to Example 43, Step 5 using 6-(2-amino-5-bromo-6-fluoropyridin-3-yl)-3,4-dihydroisoquinolin-1(2H)-one (93 mg, 0.277 mmol) and 1-[4-(4,4,5,5-tetramethyl-1,3,2-dioxaborolan-2-yl)phenyl]piperidine (95 mg, 0.332 mmol) afforded the title compound (77 mg, 66%) as a tan solid. ¹H NMR (500 MHz, DMSO-d₆) δ 7.94 (br s, 1H), 7.90 (d, J=7.8 Hz, 1H), 7.58 (d, J=10.3 Hz, 1H), 7.48-7.45 (m, 1H), 7.44 (s, 1H), 7.38 (d, J=7.8 Hz, 2H), 6.95 (d, J=8.9 Hz, 2H), 6.16 (s, 2H), 3.40 (td, J=6.5, 2.8 Hz, 2H), 3.19-3.14 (m, 4H), 2.95 (t, J=6.5 Hz, 2H), 1.64-1.58 (m, 4H), 1.57-1.51 (m, 2H); LCMS: [M+H]⁺=418.37.

### Example 61: 6-(2-amino-5-(2-chloro-4-(4-methylpiperazin-1-yl)phenyl)-6-fluoropyridin-3-yl)-3,4-dihydroisoquinolin-1(2H)-one (I-61)

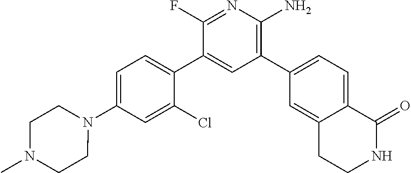 [SMILES: CN1CCN(c2ccc(-c3cc(-c4ccc5c(c4)CCNC5=O)c(N)nc3F)c(Cl)c2)CC1]

A procedure analogous to Example 43, Step 5 using 6-(2-amino-5-bromo-6-fluoropyridin-3-yl)-3,4-dihydroisoquinolin-1(2H)-one (95 mg, 0.283 mmol) and 2-chloro-4-(N-methylpiperazin-1-yl)phenylboronic acid (86 mg, 0.339 mmol) to provide the title compound (78 mg, 59%) as a white solid. ¹H NMR (500 MHz, DMSO-d₆) δ 7.93 (br s, 1H), 7.89 (d, J=7.8 Hz, 1H), 7.45-7.38 (m, 3H), 7.24 (d, J=8.7 Hz, 1H), 7.03 (d, J=2.3 Hz, 1H), 6.95 (dd, J=8.7, 2.4 Hz, 1H), 6.28 (s, 2H), 3.39 (td, J=6.5, 2.6 Hz, 2H), 3.21-3.18 (m, 4H), 2.94 (t, J=6.5 Hz, 2H), 2.45-2.42 (m, 4H), 2.22 (s, 3H); LCMS: [M+1]⁺=465.99.

### Example 62: 6-(2-amino-6-fluoro-5-(4-(methylsulfonyl)phenyl)pyridin-3-yl)-3,4-dihydroisoquinolin-1(2H)-one (I-62)

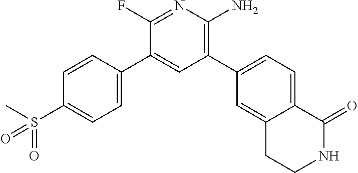 [SMILES: CS(=O)(=O)c1ccc(-c2cc(-c3ccc4c(c3)CCNC4=O)c(N)nc2F)cc1]

A procedure analogous to Example 43, Step 5 using 6-(2-amino-5-bromo-6-fluoropyridin-3-yl)-3,4-dihydroisoquinolin-1(2H)-one (105 mg, 0.312 mmol) and 4-[(methylsulfonyl)phenylboronic acid (75 mg, 0.375 mmol) to provide the title compound (0.245 mmol) in 78% yield as a tan solid. ¹H NMR (500 MHz, DMSO-d₆) δ 8.00-7.89 (m, 4H), 7.85 (br d, J=7.3 Hz, 2H), 7.76 (br d, J=9.7 Hz, 1H), 7.53-7.42 (m, 2H), 6.54 (br s, 2H), 3.41 (br s, 2H), 3.24 (br s, 3H), 2.96 (br s, 2H); LCMS: [M+H]⁺=412.19.

### Example 63: 6-(2-amino-5-(4-(cyclopropylsulfonyl)phenyl)-6-fluoropyridin-3-yl)-3,4-dihydroisoquinolin-1(2H)-one (I-63)

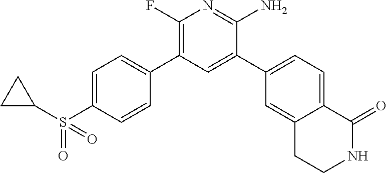 [SMILES: Nc1nc(F)c(-c2ccc(S(=O)(=O)C3CC3)cc2)cc1-c1ccc2c(c1)CCNC2=O]

A procedure analogous to Example 43, Step 5 using 6-(2-amino-5-bromo-6-fluoropyridin-3-yl)-3,4-dihydroisoquinolin-1(2H)-one (87 mg, 0.259 mmol) and 4-(cyclopropylsulfonyl)phenylboronic acid (70 mg, 0.311 mmol) afforded the title compound (98 mg, 83%) as a tan solid. ¹H NMR (500 MHz, DMSO-d₆) δ 7.95 (br s, 1H), 7.93-7.89 (m, 3H), 7.86-7.82 (m, 2H), 7.77 (d, J=10.1 Hz, 1H), 7.49 (d, J=8.1 Hz, 1H), 7.46 (s, 1H), 6.54 (s, 2H), 3.41 (td, J=6.4, 2.7 Hz, 2H), 2.95 (br t, J=6.5 Hz, 2H), 2.88 (tt, J=7.9, 4.8 Hz, 1H), 1.16-1.11 (m, 2H), 1.08-1.02 (m, 2H); LCMS: [M+H]⁺=438.30.

### Example 64: 4-(6-amino-2-fluoro-5-(1-oxo-1,2,3,4-tetrahydroisoquinolin-6-yl)pyridin-3-yl)-N,N-dimethylbenzenesulfonamide (1-64)

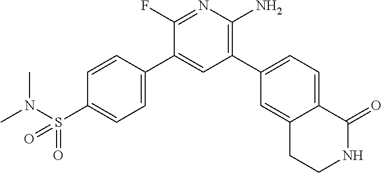 [SMILES: CN(C)S(=O)(=O)c1ccc(-c2cc(-c3ccc4c(c3)CCNC4=O)c(N)nc2F)cc1]

A procedure analogous to Example 43, Step 5 using 6-(2-amino-5-bromo-6-fluoropyridin-3-yl)-3,4-dihydroisoquinolin-1(2H)-one (98 mg, 0.292 mmol) and 4-(diethylsulfamoyl)benzeneboronic acid (84 mg, 0.328 mmol) afforded the title compound (120 mg, 93%) as a tan solid. ¹H NMR (500 MHz, DMSO-d₆) δ 7.95 (br s, 1H), 7.92 (d, J=7.9 Hz, 1H), 7.84 (d, J=8.1 Hz, 2H), 7.77 (d, J=8.7 Hz, 3H), 7.48 (d, J=7.9 Hz, 1H), 7.46 (s, 1H), 6.54 (s, 2H), 3.41 (td, J=6.4, 2.8 Hz, 2H), 2.95 (br t, J=6.5 Hz, 2H), 2.63 (s, 6H); LCMS: [M +1]⁺=441.31.

### Example 65: 4-(6-amino-2-fluoro-5-(1-oxo-1,2,3,4-tetrahydroisoquinolin-6-yl)pyridin-3-yl)-N,N-diethylbenzenesulfonamide (1-65)

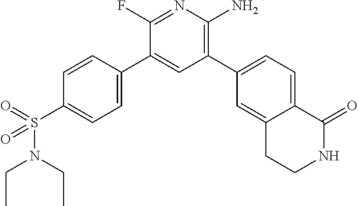 [SMILES: CCN(CC)S(=O)(=O)c1ccc(-c2cc(-c3ccc4c(c3)CCNC4=O)c(N)nc2F)cc1]

A procedure analogous to Example 43, Step 5 using 6-(2-amino-5-bromo-6-fluoropyridin-3-yl)-3,4-dihydroisoquinolin-1(2H)-one (92 mg, 0.274 mmol) and 4-(diethylsulfamoyl)benzeneboronic acid (84 mg, 0.328 mmol) afforded the title compound (130 mg, 83%) as a tan solid. ¹H NMR (500 MHz, DMSO-d₆) δ 7.95 (br s, 1H), 7.91 (d, J=7.9 Hz, 1H), 7.82-7.77 (m, 4H), 7.75 (d, J=10.1 Hz, 1H), 7.48 (br d, J=7.9 Hz, 1H), 7.46 (s, 1H), 6.52 (s, 2H), 3.41 (td, J=6.4, 2.6 Hz, 2H), 3.18 (q, J=7.1 Hz, 4H), 2.95 (br t, J=6.5 Hz, 2H), 1.06 (t, J=7.1 Hz, 6H); LCMS: [M+1]⁺=469.22.

### Example 66: 6-(2-amino-6-fluoro-5-(4-((1R,5S)-3-(tetrahydro-2H-pyran-4-yl)-3-azabicyclo[3.1.0]hexan-1-yl)phenyl)pyridin-3-yl)-3,4-dihydroisoquinolin-1(2H)-one (I-66)

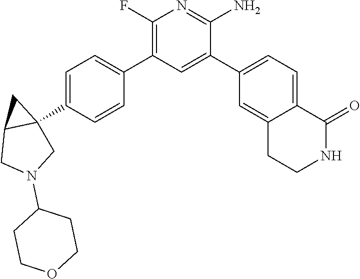 [SMILES: Nc1nc(F)c(-c2ccc([C@@]34C[C@@H]3CN(C3CCOCC3)C4)cc2)cc1-c1ccc2c(c1)CCNC2=O]

Step 1: (1R, 2S)-1-(4-bromophenyl)-2-(hydroxymethyl)cyclopropane-1-carbonitrile:

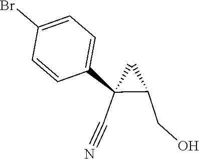 [SMILES: N#C[C@]1(c2ccc(Br)cc2)C[C@@H]1CO]

To a stirred solution of 2-(4-bromophenyl)acetonitrile (10 g, 51 mmol) in THF (80 mL), was added NaHMDS (63.7 mL, 127.5 mmol, 2M in THF) at −10° C. The mixture was stirred for 10 min, then epichlorohydrin (5.19 g, 56.1 mmol) was added dropwise at −10° C. The reaction mixture was stirred at RT for 4 h, then quenched with 2:1 dioxane: conc. HCl (10 mL) and extracted with Et₂O (3×100 mL). The combined organic layers were dried over Na₂SO₄ and concentrated under reduced pressure to afford the product (12 g, quantitative yield) as a pale yellow semi solid. LCMS: [M+H]⁺=253.23.

Step 2: ((1S, 2R)-2-(aminomethyl)-2-(4-bromophenyl) cyclopropyl) methanol:

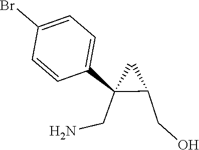 [SMILES: NC[C@]1(c2ccc(Br)cc2)C[C@@H]1CO]

To a stirred solution of (1R, 2S)-1-(4-bromophenyl)-2-(hydroxymethyl)cyclopropane-1-carbonitrile (crude 7.1 g, 28.2 mmol) in THF (70 mL) was added BF₃·OEt₂ (5.6 mL, 5.6 mmol) at RT. The reaction mixture was heated to 80° C., then added BH₃ DMS (70.7 mL, 70.7 mmol, 1M in THF). The reaction mixture was maintained at 80° C. for 4 h. Then, the reaction mixture was quenched with 10% aq.HCl (30 mL), washed with diethyl ether (2×200 mL) and the aqueous layer was neutralized with solid NaHCO₃ up to PH-8 and extracted with EtOAc (3×200 mL), the combined organic layer was wash with brine solution (200 mL), dried over with Na₂SO₄, filtered and concentrated under reduced pressure to afford the product (4.5 g, 62%) as pale yellow semi solid. LCMS: [M+H]⁺=256.26.

Step 3: (1R,5S)-1-(4-bromophenyl)-3-azabicyclo[3.1.0]hexane

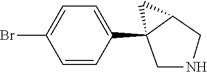 [SMILES: Brc1ccc([C@]23CNC[C@H]2C3)cc1]

To a stirred solution of ((1S, 2R)-2-(aminomethyl)-2-(4-bromophenyl) cyclopropyl) methanol (4 g, 15.6 mmol) in DCM (40 mL) was added thionyl chloride (3.5 mL, 47.0 mmol) dropwise at 0° C. The reaction mixture was stirred for 16 h at RT and washed with CH₂Cl₂ (2×100 mL). The aqueous layer was basified with sat. aqueous Na₂CO₃ solution to pH 8, extracted with 10% MeOH in DCM (3×200 mL), and the combined organic layers were dried over Na₂SO₄ and concentrated to gave crude product. Trituration with EtOAc afforded the product (3 g, 81%) as a white solid. LCMS: [M+H]⁺=238.17.

Step 4: (1R, 5S)-1-(4-bromophenyl)-3-(tetrahydro-2H-pyran-4-yl)-3-azabicyclo [3.1.0]hexane (5):

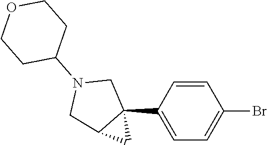 [SMILES: Brc1ccc([C@@]23C[C@@H]2CN(C2CCOCC2)C3)cc1]

A solution of (1R,5S)-1-(4-bromophenyl)-3-azabicyclo[3.1.0]hexane (4 g, 16.8 mmol) and tetrahydro-4H-pyran-4-one (2.53 g, 25.3 mmol) in MeOH: ACOH (76:4 mL) was stirred for 30 min, then NaCNBH₃ (2.1 g, 33.7 mmol) was added portionwise at RT. The reaction mixture was stirred at RT for 16 h, then concentrated and diluted with water and basified with aq sat.NaHCO₃ solution to pH=8. The aqueous layer was separated and extracted with EtOAc (3×200 mL). The combined organic layers were dried over Na₂SO₄ and concentrated to give crude compound. Purification by column chromatography using 100-200 silica gel as an eluent EtOAc afforded the product (2 g, 37%) as an off-white semi solid. LCMS: [M+H]⁺=322.37

Step 5: (1R,5S)-3-(tetrahydro-2H-pyran-4-yl)-1-(4-(4,4,5,5-tetramethyl-1,3,2-dioxaborolan-2-yl)phenyl)-3-azabicyclo[3.1.0]hexane:

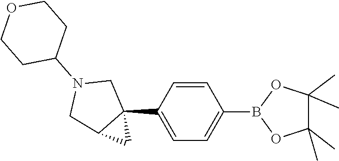 [SMILES: CC1(C)OB(c2ccc([C@@]34C[C@@H]3CN(C3CCOCC3)C4)cc2)OC1(C)C]

To a stirred solution of (1R, 5S)-1-(4-bromophenyl)-3-(tetrahydro-2H-pyran-4-yl)-3-azabicyclo [3.1.0] hexane (5) (1 g, 3.1 mmol) and 2-isopropoxy-4,4,5,5-tetramethyl-1,3,2-dioxaborolane (3.8 mL, 18.6 mmol) in THF (50 mL) was added dropwise n-BuLi (11.6 mL, 18.6 mmol) at −78° C. The reaction mixture was maintained at RT for 6 h, quenched with aq sat. NH₄Cl solution and extracted with EtOAc (3×50 mL). The combined organic layers were washed with brine solution (100 mL), dried over with Na₂SO₄, filtered and concentrated under reduced pressure to give crude compound. The crude compound was purified by prep HPLC to afford the product (0.2 g, 17%) as a white solid, which consisted of a mixture of boronic acid as well as boronate ester. ¹H NMR (400 MHz, DMSO-d₆): δ 7.57 (d, J=7.6 Hz, 2H), δ 7.14 (d, J=7.2 Hz, 2H), δ 3.82 (d, J=9.6 Hz, 4H), δ 3.36-3.26 (m, 4H), δ 3.06 (d, J=8.4 Hz, 2H), δ 2.55 (d, J=7.6 Hz, 2H), δ 2.42 (d, J=6.0 Hz, 1H), δ 2.31 (s, 1H), δ 1.83-1.70 (m, 2H), δ 1.43-1.22 (m, 12H); LCMS: [M+H]⁺=370.32.

Step 6: 6-(2-amino-6-fluoro-5-(4-((1R,5S)-3-(tetrahydro-2H-pyran-4-yl)-3-azabicyclo[3.1.0]hexan-1-yl)phenyl)pyridin-3-yl)-3,4-dihydroisoquinolin-1(2H)-one

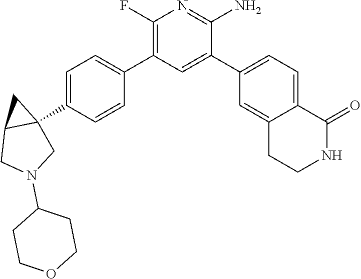 [SMILES: Nc1nc(F)c(-c2ccc([C@@]34C[C@@H]3CN(C3CCOCC3)C4)cc2)cc1-c1ccc2c(c1)CCNC2=O]

A procedure analogous to Example 1, General Method A using (1R,5S)-3-(tetrahydro-2H-pyran-4-yl)-1-(4-(4,4,5,5-tetramethyl-1,3,2-dioxaborolan-2-yl)phenyl)-3-azabicyclo[3.1.0]hexane (69 mg, 0.187 mmol), 6-(2-amino-5-bromo-6-fluoropyridin-3-yl)-3,4-dihydroisoquinolin-1(2H)-one (62.8 mg, 0.187 mmol), PdCl₂dppf (19.14 mg, 0.026 mmol), Cs₂CO₃ (183 mg, 0.561 mmol), H₂O (2 mL) and 1,4-dioxane (4 mL) afforded the title compound as a light tan solid (57 mg, 60% yield based on purity of 98%). ¹H NMR (500 MHz, DMSO-d₆) δ 7.92-7.97 (m, 1H), 7.91 (d, J=8.0 Hz, 1H), 7.62 (d, J=10.2 Hz, 1H), 7.43-7.50 (m, 4H), 7.20 (d, J=8.2 Hz, 2H), 6.29 (s, 2H), 3.78-3.89 (m, 2H), 3.36-3.46 (m, 3H), 3.27-3.31 (m, 1H), 3.04-3.16 (m, 1H), 2.96 (br t, J=6.5 Hz, 2H), 2.53-2.62 (m, 2), 2.38-2.48 (m, 1H), 2.28-2.36 (m, 1H), 1.70-1.87 (m, 3H), 1.27-1.43 (m, 3H), 0.73-0.81 (m, 1H); LCMS: [M+H]⁺=499.41.

### Example 67: 7-(2-amino-5-(4-(4-ethylpiperazin-1-yl)phenyl)-6-fluoropyridin-3-yl)-2,3,4,5-tetrahydro-1H-benzo[c]azepin-1-one (I-67)

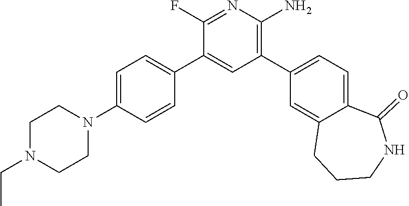 [SMILES: CCN1CCN(c2ccc(-c3cc(-c4ccc5c(c4)CCCNC5=O)c(N)nc3F)cc2)CC1]

Step 1: 3-chloro-5-(4-(4-ethylpiperazin-1-yl)phenyl)-6-fluoropyridin-2-amine

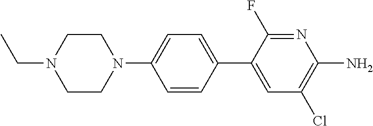 [SMILES: CCN1CCN(c2ccc(-c3cc(Cl)c(N)nc3F)cc2)CC1]

A method similar to Example 1, General Method A heating a sealed and degassed mixture of 5-bromo-3-chloro-6-fluoropyridin-2-amine (70 mg, 0.310 mmol), (4-(4-ethylpiperazin-1-yl)phenyl)boronic acid pinacol ester (98 mg, 0.310 mmol), PdCl₂dppf (22.7 mg, 0.031 mmol), Cs₂CO₃ (303 mg, 0.931 mmol) in H₂O (2 mL) and DME (3 mL) under microwave irradiation at 90° C. for 1 h afforded the product as a brown solid (82 mg, 79%). LCMS: [M+H]⁺=335.00.

Step 2: 7-(2-amino-5-(4-(4-ethylpiperazin-1-yl)phenyl)-6-fluoropyridin-3-yl)-2,3,4,5-tetrahydro-1H-benzo[c]azepin-1-one

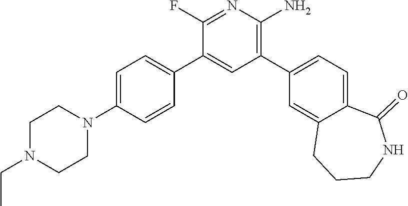 [SMILES: CCN1CCN(c2ccc(-c3cc(-c4ccc5c(c4)CCCNC5=O)c(N)nc3F)cc2)CC1]

A procedure analogous to Example 43, Step 5 using 7-(tetramethyl-1, 3,2-dioxaborolan-2-yl)-2,3,4,5-tetrahydro-1H-2-benzazepin-1-one (77 mg, 0.27 mmol), K₃PO₄ (156 mg, 0.735 mmol), XPhos Pd G2 (19.3 mg, 0.024 mmol), H₂O (6 mL), 3-chloro-5-(4-(4-ethylpiperazin-1-yl)phenyl)-6-fluoropyridin-2-amine (82 mg, 0.24 mmol) and MeCN (9 mL) afforded the product as a white solid (5 mg, 4% based on purity of 96%). ¹H NMR (500 MHz, DMSO-d₆) δ 8.18-8.28 (m, 2H), 7.75 (d, J=8.1 Hz, 1H), 7.56 (dd, J=8.1, 1.8 Hz, 1H), 7.52 (d, J=1.5 Hz, 1H), 7.47 (br d, J=7.5 Hz, 2H), 7.05 (d, J=8.8 Hz, 2H), 3.15-3.27 (m, 4H), 2.93-3.09 (m, 2H), 2.74-2.90 (m, 2H), 2.53-2.57 (m, 4H), 2.38 (q, J=7.0 Hz, 2H), 1.90-2.01 (m, 2H), 1.05 (t, J=7.2 Hz, 3H); LCMS: [M+H]⁺=460.36.

### Example 68: 6-(2-amino-6-fluoro-5-(4-(4-methylpiperazin-1-yl)-3-(trifluoromethyl)phenyl)pyridin-3-yl)-3,4-dihydroisoquinolin-1(2H)-one (I-68)

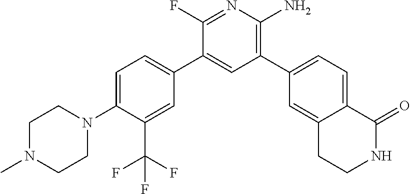 [SMILES: CN1CCN(c2ccc(-c3cc(-c4ccc5c(c4)CCNC5=O)c(N)nc3F)cc2C(F)(F)F)CC1]

To a solution of 1-methyl-4-(4-(4,4,5,5-tetramethyl-1,3,2-dioxaborolan-2-yl)-2-(trifluoromethyl)phenyl)piperazine (65.5 mg, 0.177 mmol) and 6-(2-amino-5-bromo-6-fluoropyridin-3-yl)-3,4-dihydroisoquinolin-1(2H)-one (54.1 mg, 0.161 mmol) in 1,4-dioxane (3 mL) was added a solution of Cs₂CO₃ (157 mg, 0.483 mmol) in water (1 mL) at RT and the reaction mixture was degassed under argon for 15 min. Then PddppfCl2 (17.66 mg, 0.024 mmol) was added and the reaction mixture was heated at 90° C. for 6 h. The reaction mixture was dry loaded onto Celite® and purified by flash chromatography (0-20%, DCM/MeOH) to afford the title compound (66.2 mg, 78%) as a light brown powder. ¹H NMR (500 MHz, DMSO-d₆) δ 7.95 (br s, 1H), 7.90 (d, J=7.8 Hz, 1H), 7.82 (br d, J=8.4 Hz, 1H), 7.78 (s, 1H), 7.71 (d, J=10.3 Hz, 1H), 7.58 (d, J=8.3 Hz, 1H), 7.47 (br d, J=8.1 Hz, 1H), 7.45 (s, 1H), 6.40 (s, 2H), 3.40 (br d, J=2.3 Hz, 2H), 2.95 (s, 2H), 2.93-2.87 (m, 4H), 2.26 (br s, 3H), 4H under the water peak; LCMS: [M+H]⁺=500.16.

### Example 69: 6-(2-amino-5-(4-(4-ethylpiperazin-1-yl)phenyl)-6-fluoropyridin-3-yl)-4,4-dimethyl-3,4-dihydroisoquinolin-1(2H)-one (I-69)

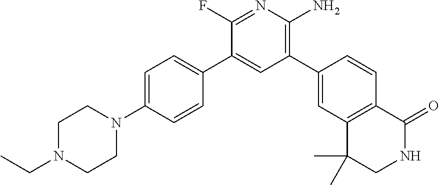 [SMILES: CCN1CCN(c2ccc(-c3cc(-c4ccc5c(c4)C(C)(C)CNC5=O)c(N)nc3F)cc2)CC1]

A procedure similar to Example 1, General Method A using 6-(2-amino-5-bromo-6-fluoropyridin-3-yl)-4,4-dimethyl-3,4-dihydroisoquinolin-1(2H)-one (132 mg, 0.301 mmol, purity of 83%), (4-(4-ethylpiperazin-1-yl)phenyl)boronic acid pinacol ester (100 mg, 0.316 mmol), PdCl₂dppf (22.0 mg, 0.030 mmol), Cs₂CO₃ (294 mg, 0.902 mmol), H₂O (6 mL) and DME (12 mL) afforded the title compound as an off white solid (26 mg, 18% based on purity of 96%). ¹H NMR (500 MHz, DMSO-d₆) δ 7.98 (br s, 1H), 7.89-7.96 (m, 1H), 7.59 (d, J=10.3 Hz, 1H), 7.45-7.53 (m, 2H), 7.41 (br d, J=8.0 Hz, 2H), 6.98 (d, J=8.9 Hz, 2H), 6.14 (s, 2H), 3.11-3.24 (m, 7H), 2.31-2.44 (m, 2H), 1.32 (s, 6H), 1.04 (t, J=7.2 Hz, 3H). Signals due to 3 H are obscured by H₂O peak and/or the solvent peak. LCMS: [M+H]⁺=474.35.

### Example 70: 6-(2-amino-5-(4-(4-ethylpiperazin-1-yl)phenyl)-6-fluoropyridin-3-yl)-3,3-dimethyl-3,4-dihydroisoquinolin-1(2H)-one 2,2,2-trifluoroacetate (1-70)

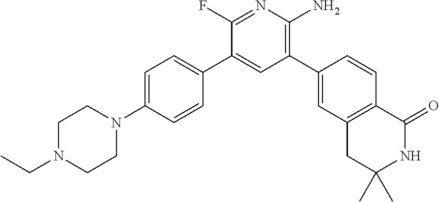 [SMILES: CCN1CCN(c2ccc(-c3cc(-c4ccc5c(c4)CC(C)(C)NC5=O)c(N)nc3F)cc2)CC1]

A procedure analogous to Example 1, General Method A using 6-(2-amino-5-bromo-6-fluoropyridin-3-yl)-3,3-dimethyl-3,4-dihydroisoquinolin-1(2H)-one (192 mg, 0.285 mmol, 54%), (4-(4-ethylpiperazin-1-yl)phenyl)boronic acid pinacol ester (95 mg, 0.30 mmol), PdCl₂dppf (20.8 mg, 0.028 mmol), Cs₂CO₃ (278 mg, 0.854 mmol), H₂O (6 mL), DME (12 mL) afforded the title compound as a pale yellow solid (67 mg, 39% yield based on purity of 98%). ¹H NMR (500 MHz, CD₃OD) δ ppm 8.05 (d, J=8.0 Hz, 1H), 7.64 (d, J=9.8 Hz, 1H), 7.47-7.54 (m, 3H), 7.40-7.47 (m, 1H), 7.10 (d, J=8.8 Hz, 2H), 3.93 (br d, J=12.5 Hz, 2H), 3.70 (br d, J=10.5 Hz, 2H), 3.28-3.32 (m, 2H), 3.19-3.27 (m, 2H), 3.07-3.16 (m, 2H), 3.02-3.05 (m, 2H), 1.42 (t, J=7.3 Hz, 3H), 1.36 (s, 6H). LCMS: [M+H]⁺=474.05.

### Example 71: 6-(2-amino-5-(4-((1R,5S)-3-ethyl-3-azabicyclo[3.1.0]hexan-1-yl)phenyl)-6-fluoropyridin-3-yl)-3,4-dihydroisoquinolin-1(2H)-one (I-71)

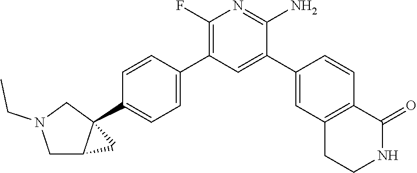 [SMILES: CCN1C[C@H]2C[C@@]2(c2ccc(-c3cc(-c4ccc5c(c4)CCNC5=O)c(N)nc3F)cc2)C1]

6-(2-amino-5-(4-((1R,5S)-3-ethyl-3-azabicyclo[3.1.0]hexan-1-yl)phenyl)-6-fluoropyridin-3-yl)-3,4-dihydroisoquinolin-1(2H)-one
Step 1: (1R,5S)-1-(4-bromophenyl)-3-ethyl-3-azabicyclo[3.1.0]hexane

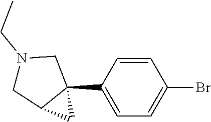

A procedure analogous to Example 53, Step 1 using (1R,5S)-1-(4-bromophenyl)-3-azabicyclo[3.1.0]hexane (113 mg, 0.475 mmol, preparation described in Example 66, Step 3) and bromoethane (0.053 ml, 0.712 mmol) afforded the product as a white powder (94 mg, 74%). LCMS: [M+H]⁺=266.08.

Step 2: 6-(2-Amino-5-(4-((1R,5S)-3-ethyl-3-azabicyclo[3.1.0]hexan-1-yl)phenyl)-6-fluoropyridin-3-yl)-3,4-dihydroisoquinolin-1(2H)-one

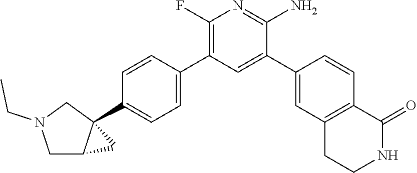 [SMILES: CCN1C[C@H]2C[C@@]2(c2ccc(-c3cc(-c4ccc5c(c4)CCNC5=O)c(N)nc3F)cc2)C1]

A procedure analogous to Example 43, Step 5 using (1R,5S)-1-(4-bromophenyl)-3-ethyl-3-azabicyclo[3.1.0]hexane (94 mg, 0.353 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (176 mg, 0.459 mmol) afforded the title compound as a light brown oil (64 mg, 39%). ¹H NMR (500 MHz, DMSO-d₆) δ 7.9-8.0 (m, 1H), 7.91 (d, J=7.9 Hz, 1H), 7.6-7.6 (m, 1H), 7.4-7.5 (m, 4H), 7.19 (d, J=8.4 Hz, 2H), 6.29 (s, 2H), 3.4-3.5 (m, 2H), 3.0-3.1 (m, 1H), 2.9-3.0 (m, 2H), 2.4-2.5 (m, 4H), 2.4-2.4 (m, 1H), 1.7-1.8 (m, 1H), 1.3-1.4 (m, 1H), 1.0-1.1 (m, 3H), 0.7-0.8 (m, 1H); LCMS: [M+H]⁺=443.41.

### Example 72: 6-(2-amino-5-(4-((1R,5S)-3-(2,2-difluoroethyl)-3-azabicyclo[3.1.0]hexan-1-yl)phenyl)-6-fluoropyridin-3-yl)-3,4-dihydroisoquinolin-1(2H)-one (I-72)

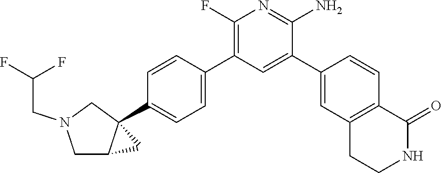 [SMILES: Nc1nc(F)c(-c2ccc([C@@]34C[C@@H]3CN(CC(F)F)C4)cc2)cc1-c1ccc2c(c1)CCNC2=O]

Step 1: (1R,5S)-1-(4-bromophenyl)-3-(2,2-difluoroethyl)-3-azabicyclo[3.1.0]hexane

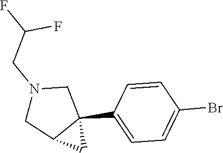 [SMILES: FC(F)CN1C[C@H]2C[C@@]2(c2ccc(Br)cc2)C1]

A procedure analogous to Example 53, Step 1 using (1R,5S)-1-(4-bromophenyl)-3-azabicyclo[3.1.0]hexane (107 mg, 0.449 mmol) and 2,2-difluoroethyl trifluoromethanesulfonate (0.120 ml, 0.899 mmol) at RT overnight. The reaction mixture was concentrated to Celite® and purified by reverse phase chromatography (C18; 0-60% acetonitrile/water) to afford the product as a light brown oil (87 mg, 64%). LCMS: [M+H]⁺=302.00

Step 2: 6-(2-amino-5-(4-((1R,5S)-3-(2,2-difluoroethyl)-3-azabicyclo[3.1.0]hexan-1-yl)phenyl)-6-fluoropyridin-3-yl)-3,4-dihydroisoquinolin-1(2H)-one

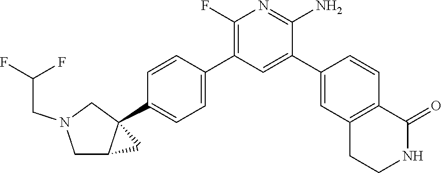 [SMILES: Nc1nc(F)c(-c2ccc([C@@]34C[C@@H]3CN(CC(F)F)C4)cc2)cc1-c1ccc2c(c1)CCNC2=O]

A procedure analogous to that of Example 43, Step 5 using (1R,5S)-1-(4-bromophenyl)-3-(2,2-difluoroethyl)-3-azabicyclo[3.1.0]hexane (87 mg, 0.288 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (143 mg, 0.374 mmol)) afforded the title compound as a beige solid (50 mg, 35%). ¹H NMR (500 MHz, DMSO-d₆) δ 7.9-8.0 (m, 1H), 7.91 (d, 1H, J=7.8 Hz), 7.6-7.6 (m, 1H), 7.4-7.5 (m, 4H), 7.1-7.2 (m, 2H), 6.3-6.4 (m, 2H), 6.0-6.2 (m, 1H), 3.4-3.4 (m, 3H), 3.1-3.1 (m, 1H), 2.9-3.0 (m, 4H), 2.7-2.8 (m, 1H), 2.6-2.7 (m, 1H), 1.84 (td, J=7.8, 3.9 Hz, 1H), 1.3-1.3 (m, 1H), 0.7-0.8 (m, 1H); LCMS: [M+H]⁺=479.45.

### Example 73: 6-(2-amino-5-(4-(1-(2,2-difluoroethyl)-1,2,3,6-tetrahydropyridin-4-yl)phenyl)-6-fluoropyridin-3-yl)-3,4-dihydroisoquinolin-1(2H)-one (I-73)

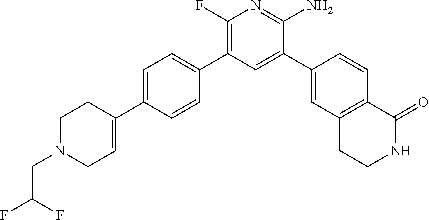 [SMILES: Nc1nc(F)c(-c2ccc(C3=CCN(CC(F)F)CC3)cc2)cc1-c1ccc2c(c1)CCNC2=O]

To a slurry of 6-(2-amino-6-fluoro-5-(4-(1,2,3,6-tetrahydropyridin-4-yl)phenyl)pyridin-3-yl)-3,4-dihydroisoquinolin-1(2H)-oneTFA (40 mg, 0.076 mmol, preparation described in Example 57) in DMF (2 mL) at RT was added 2,2-difluoroethyl trifluoromethanesulfonate (0.020 mL, 0.151 mmol) and Et₃N (0.105 mL, 0.757 mmol) and the mixture was stirred for 16 h at RT. The reaction mixture was partitioned between water and CHCl₃/IPA 4:1 mix. The layers were separated and the aqueous layer was extracted with CHCl₃/IPA 4:1 mix (2×3 mL). The combined organic extracts were dried and concentrated onto Celite®. Reverse phase chromatography (C18; 0-35% acetonitrile/water) afforded the title compound as a beige solid (8 mg, 20% yield based on purity of 90%). ¹H NMR (500 MHz, DMSO-d₆) δ 7.97-7.90 (m, 2H), 7.69-7.65 (m, 1H), 7.56-7.51 (m, 2H), 7.50-7.44 (m, 4H), 6.36-6.07 (m, 4H), 3.44-3.40 (m, 2H), 3.29-3.22 (m, 2H), 2.99-2.95 (m, 2H), 2.91-2.82 (m, 2H), 2.82-2.76 (m, 2H), 2.54-2.52 (m, 2H); LCMS: [M+H]⁺=479.45.

### Example 74: 6-(2-amino-6-chloro-5-(4-(4-methylpiperazin-1-yl)phenyl)pyridin-3-yl)-3,4-dihydroisoquinolin-1(2H)-one (I-74)

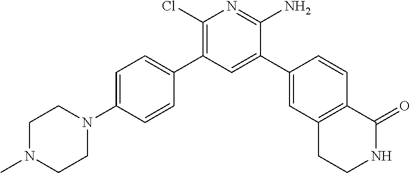 [SMILES: CN1CCN(c2ccc(-c3cc(-c4ccc5c(c4)CCNC5=O)c(N)nc3Cl)cc2)CC1]

Step 1: 6-(2-Amino-5-bromo-6-chloropyridin-3-yl)-3,4-dihydroisoquinolin-1(2H)-one

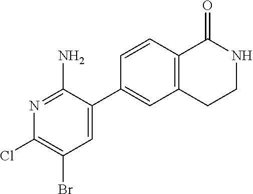 [SMILES: Nc1nc(Cl)c(Br)cc1-c1ccc2c(c1)CCNC2=O]

To a microwave vial containing a suspension of 5-bromo-6-chloro-3-iodopyridin-2-amine (121 mg, 0.362 mmol), 3,4-dihydro-1(2H)-isoquinolinone-6-boronic acid pinacol ester (90 mg, 0.330 mmol), K₂CO₃ (96 mg, 0.692 mmol) and bis(triphenylphosphine)palladium(II) dichloride (27.8 mg, 0.040 mmol) in DMF (9 mL) was added water (1 mL). The vial was flushed with argon, sealed and then heated in a microwave reactor at 80° C. for 1 h. The reaction mixture was partitioned between water and EtOAc. The organic phase was separated and aqueous phase was further extracted with DCM (2×3 mL). The combined org phase was dried and concentrated onto Celite®. Silica gel chromatography (eluting with 0-60% EtOAc/Hexanes) afforded the title compound as an off white solid (60 mg, 52%). LCMS: [M+H]⁺=354.21

Step 2: 6-(2-amino-6-chloro-5-(4-(4-methylpiperazin-1-yl)phenyl)pyridin-3-yl)-3,4-dihydroisoquinolin-1(2H)-one:

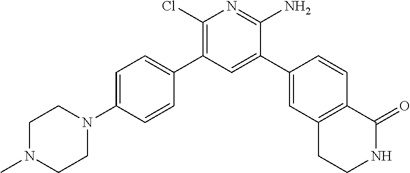 [SMILES: CN1CCN(c2ccc(-c3cc(-c4ccc5c(c4)CCNC5=O)c(N)nc3Cl)cc2)CC1]

A procedure analogous to that of Example 43, Step 5, by coupling 6-(2-amino-5-bromo-6-chloropyridin-3-yl)-3,4-dihydroisoquinolin-1(2H)-one (29 mg, 0.082 mmol) and 4-(4-methylpiperazin-1-yl)phenylboronic acid, pinacol ester (34.8 mg, 0.115 mmol) at 90° C. for 1.75 h. The reaction mixture was partitioned between brine and EtOAc. The organic phase was separated and aqueous phase was further extracted with EtOAc (2×3 mL). The combined organic layers were dried over Na₂SO₄ and concentrated onto Celite®. Reverse phase chromatography (C18; 0-30% acetonitrile/water), afforded the title compound as a yellow powder (19 mg, 39%). ¹H NMR (500 MHz, DMSO-d₆) δ 7.97-7.92 (m, 1H), 7.89 (d, J=7.9 Hz, 1H), 7.48-7.43 (m, 2H), 7.38-7.34 (m, 1H), 7.34-7.27 (m, 2H), 7.00-6.94 (m, 2H), 6.22 (s, 2H), 3.43-3.37 (m, 2H), 3.19-3.15 (m, 4H), 2.94 (br t, J=6.5 Hz, 2H), 2.47-2.43 (m, 4H), 2.24-2.21 (m, 3H); LCMS: [M+H]⁺=448.48.

### Example 75: 6-(2-amino-6-chloro-5-(4-(4-ethylpiperazin-1-yl)phenyl)pyridin-3-yl)-3,4-dihydroisoquinolin-1(2H)-one (I-75)

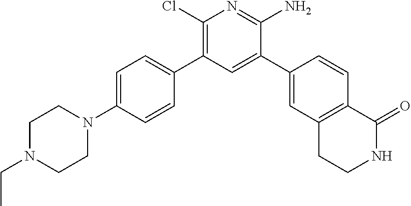 [SMILES: CCN1CCN(c2ccc(-c3cc(-c4ccc5c(c4)CCNC5=O)c(N)nc3Cl)cc2)CC1]

A procedure analogous to that of Example 43, Step 5, using 6-(2-amino-5-bromo-6-chloropyridin-3-yl)-3,4-dihydroisoquinolin-1(2H)-one (25 mg, 0.071 mmol), prepared as per Example 74, Step 1) and (4-(4-ethylpiperazin-1-1)phenyl)boronic acid pinacol ester (31.4 mg, 0.099 mmol) at 90° C. for 1.75 h afforded the title compound as a pale yellow solid (16 mg, 46%). ¹H NMR (500 MHz, DMSO-d₆) δ 7.98-7.92 (m, 1H), 7.92-7.86 (m, 1H), 7.50-7.45 (m, 1H), 7.45-7.42 (m, 1H), 7.37-7.34 (m, 1H), 7.31 (brd, J=8.4 Hz, 2H), 7.06-6.90 (m, 2H), 6.33-6.17 (m, 2H), 3.42-3.38 (m, 2H), 3.20-3.13 (m, 4H), 2.97-2.91 (m, 2H), 2.53-2.51 (m, 4H), 2.37 (q, J=7.1 Hz, 2H), 1.07-1.01 (m, 3H). LCMS: [M+H]⁺=462.40.

### Example 76: 6-(2-amino-5-(4-(1-ethyl-1,2,3,6-tetrahydropyridin-4-yl)phenyl)-6-fluoropyridin-3-yl)-3,4-dihydroisoquinolin-1(2H)-one (I-76)

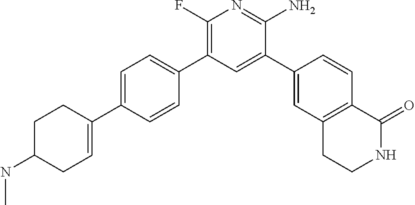 [SMILES: CNC1CC=C(c2ccc(-c3cc(-c4ccc5c(c4)CCNC5=O)c(N)nc3F)cc2)CC1]

A slurry of 6-(2-amino-6-fluoro-5-(4-(1,2,3,6-tetrahydropyridin-4-yl)phenyl)pyridin-3-yl)-3,4-dihydroisoquinolin-1(2H)-one.TFA (30 mg, 0.057 mmol), bromoethane (8.42 μl, 0.114 mmol) and Et₃N (0.079 mL, 0.568 mmol) in DMF (2 mL) was stirred overnight at RT. The reaction mixture was partitioned between water (3 mL) and CH₂Cl₂ (3 mL). The layers were separated and the aqueous layer was further extracted with CH₂Cl₂ (x2). The combined organic extracts were dried and concentrated onto Celite®. Reverse phase chromatography (C18; 0-100% acetonitrile/water) afforded the title compound as a beige solid (15.5 mg, 59%). ¹H NMR (500 MHz, DMSO-d₆) δ 7.99-7.93 (m, 1H), 7.93-7.89 (m, 1H), 7.70-7.64 (m, 1H), 7.56-7.51 (m, 2H), 7.50-7.43 (m, 4H), 6.39-6.25 (m, 2H), 6.24-6.16 (m, 1H), 3.46-3.39 (m, 2H), 3.07 (br d, J=2.6 Hz, 2H), 3.01-2.93 (m, 2H), 2.66-2.58 (m, 2H), 2.50-2.48 (m, 2H), 2.48-2.40 (m, 2H), 1.06 (t, J=7.1 Hz, 3H); LCMS: [M+H]⁺=443.41.

### Example 77: (S)-6-(2-amino-6-fluoro-5-(4-(2-isopropylmorpholino)phenyl)pyridin-3-yl)-3,4-dihydro-2,7-naphthyridin-1(2H)-one 2,2,2-trifluoroacetate (1-77)

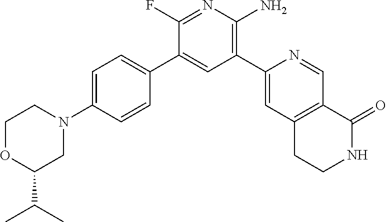 [SMILES: CC(C)[C@H]1CN(c2ccc(-c3cc(-c4cc5c(cn4)C(=O)NCC5)c(N)nc3F)cc2)CCO1]

Step 1. 6-(2-amino-6-fluoropyridin-3-yl)-3,4-dihydro-2,7-naphthyridin-1(2H)-one

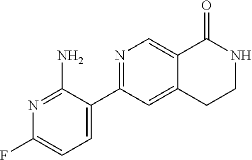 [SMILES: Nc1nc(F)ccc1-c1cc2c(cn1)C(=O)NCC2]

A microwave vial was charged with XPhos Pd G2 (110 mg, 0.139 mmol) 3-chloro-6-fluoropyridin-2-amine (120 mg, 0.819 mmol), B₂pin₂ (385 mg, 1.51 mmol), KOAc (281 mg, 2.87 mmol) and then filled with N₂. 1,4-dioxane (10 mL) was added and the reaction was degassed with N₂. The sealed vial was heated under microwave irradiation at 110° C. for 3 h. (LCMS [M+H]⁺ 238.74). The crude mixture containing 6-fluoro-3-(4,4,5,5-tetramethyl-1,3,2-dioxaborolan-2-yl)pyridin-2-amine (approx. 73% purity) was then charged with 6-bromo-3,4-dihydro-2H-[2,7]naphthyridin-1-one (149 mg, 0.658 mmol), Cs₂CO₃(584 mg, 1.79 mmol), PdCl₂dppf(43.8 mg, 0.060 mmol) and H₂O (3 mL). The content was degassed with N₂ and subsequently heated sealed under microwave irradiation for 1 h at 90° C. To drive the reaction to completion, another portion of 6-bromo-3,4-dihydro-2H-[2,7]naphthyridin-1-one (67.9 mg, 0.299 mmol) and PdCl₂dppf(43.8 mg, 0.060 mmol) was added at RT. The reaction mixture was again degassed and subjected to heating sealed in a microwave reactor at 90° C. for 1 h. The reaction mixture was concentrated under reduced pressure and purified by flash chromatography (25 g SiO₂, using MeOH in CH₂Cl₂ and later 2 M NH₃/MeOH in CH₂Cl₂), to afford the product as a beige solid (237 mg, purity of 77%). LCMS: [M+H]⁺=259.21.

Step 2.6-(2-amino-5-bromo-6-fluoropyridin-3-yl)-3,4-dihydro-2,7-naphthyridin-1(2H)-one:

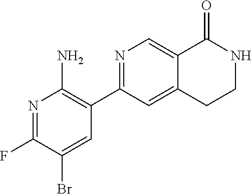 [SMILES: Nc1nc(F)c(Br)cc1-c1cc2c(cn1)C(=O)NCC2]

To a suspension of 6-(2-amino-6-fluoropyridin-3-yl)-3,4-dihydro-2,7-naphthyridin-1(2H)-one (237 mg, 0.707 mmol, 77%) in DMF (12 mL) was added NBS (132 mg, 0.742 mmol) in one portion at 0° C. The reaction was allowed to warm slowly to RT and then stirred for the total of 2 h. The mixture was concentrated to dryness in vacuo, taken into MeCN (2 mL), sonicated and left at −20° C. overnight. A filtration of the cold mixture and washing of the solid with small amounts of MeCN afforded the product as an orange-rust solid (82 mg, 31% yield based on purity of 90%). LCMS: [M+H]⁺=337.11/339.14.

Step 3. (S)-6-(2-amino-6-fluoro-5-(4-(2-isopropylmorpholino)phenyl)pyridin-3-yl)-3,4-dihydro-2,7-naphthyridin-1(2H)-one, TFA

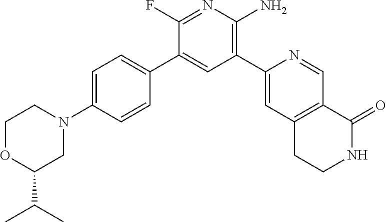 [SMILES: CC(C)[C@H]1CN(c2ccc(-c3cc(-c4cc5c(cn4)C(=O)NCC5)c(N)nc3F)cc2)CCO1]

A procedure similar to Example 1, General Method A using 6-(2-amino-5-bromo-6-fluoropyridin-3-yl)-3,4-dihydro-2,7-naphthyridin-1(2H)-one (37 mg, 0.099 mmol, 90 %), (S)-2-isopropyl-4-(4-(4,4,5,5-tetramethyl-1,3,2-dioxaborolan-2-yl)phenyl)morpholine (58.9 mg, 0.178 mmol, from Example 5, Step 2), Cs₂CO₃ (113 mg, 0.346 mmol, PdCl₂dppf (10.8 mg, 0.015 mmol), H₂O (5 mL), EtOH (7.5 mL) and DME (7.5 mL) afforded the TFA salt of the title compound as a yellow solid (20 mg, 32%). ¹H NMR (500 MHz, DMSO-d₆) δ 8.96 (s, 1H), 8.32 (d, J=10.2 Hz, 1H), 8.06-8.14 (m, 2H), 7.80-8.03 (m, 1H), 7.48 (br d, J=8.1 Hz, 2H), 7.05 (d, J=8.8 Hz, 2H), 3.99 (br dd, J=11.2, 2.3 Hz, 1H), 3.59-3.67 (m, 2H), 3.56 (br d, J=11.9 Hz, 1H), 3.22-3.36 (m, 1H), 3.01 (br t, J=6.5 Hz, 2H), 2.69 (td, J=11.8, 3.3 Hz, 1H), 2.43-2.49 (m, 1H), 1.69-1.79 (m, 1H), 0.93-1.00 (m, 6H) one signal (7.80-8.03). Separate peaks corresponding to 3 H are obscured by the H₂O and/or solvent peaks. LCMS [M+H]⁺=462.39.

### Example 78: 6-(2-amino-5-(4-(1-(2,2-difluoroethyl)piperidin-3-yl)phenyl)-6-fluoropyridin-3-yl)-3,4-dihydroisoquinolin-1(2H)-one (I-78)

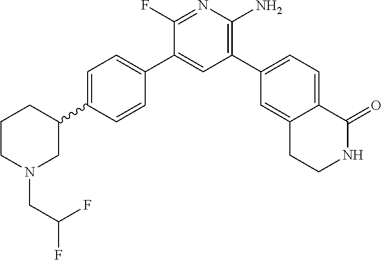 [SMILES: Nc1nc(F)c(-c2ccc(C3CCCN(CC(F)F)C3)cc2)cc1-c1ccc2c(c1)CCNC2=O]

Step 1: 6-(2-amino-6-fluoro-5-(4-(piperidin-3-yl)phenyl)pyridin-3-yl)-3,4-dihydroisoquinolin-1(2H)-one

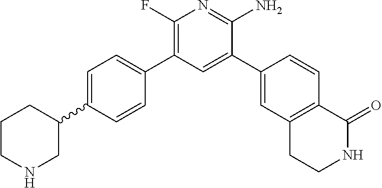 [SMILES: Nc1nc(F)c(-c2ccc(C3CCCNC3)cc2)cc1-c1ccc2c(c1)CCNC2=O]

A procedure analogous to Example 43, Step 5 using 3-(4-bromo-phenyl)-piperidine hydrochloride (175 mg, 0.633 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (315 mg, 0.823 mmol). The reaction was partitioned between water and CHCl₃IPA 4:1 mix. The layers were separated and the aqueous layer was extracted with CHCl₃/IPA 4:1 mix (2×3 mL). The combined organic extracts were dried and concentrated onto Celite®. Reverse phase chromatography (C18; 0-100% acetonitrile/water) afforded the product as a beige solid (192 mg, 66% yield based on purity of 90%). LCMS: [M+H]⁺=417.46.

Step 2: 6-(2-amino-5-(4-(1-(2,2-difluoroethyl)piperidin-3-yl)phenyl)-6-fluoropyridin-3-yl)-3,4-dihydroisoquinolin-1(2H)-one:

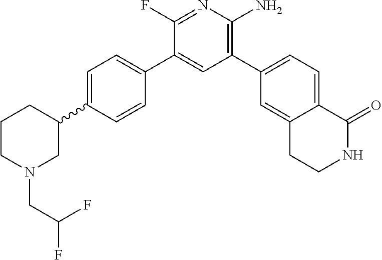 [SMILES: Nc1nc(F)c(-c2ccc(C3CCCN(CC(F)F)C3)cc2)cc1-c1ccc2c(c1)CCNC2=O]

A procedure analogous to that of Example 53, Step 1 using 6-(2-amino-6-fluoro-5-(4-(piperidin-3-yl)phenyl)pyridin-3-yl)-3,4-dihydroisoquinolin-1(2H)-one (40 mg, 0.086 mmol) and 2,2-difluoroethyl trifluoromethanesulfonate (0.023 mL, 0.173 mmol) at 80° C. for 6 h. The reaction mixture was partitioned between water (3 mL) and CH₂Cl₂ (3 mL). The layers were separated and the aqueous layer was further extracted with CH₂Cl₂ (x 2). The combined organic extracts was dried and concentrated onto Celite®. Reverse phase chromatography (C18; 0-30% acetonitrile/water) afforded the title compound as a beige solid (26 mg, 60%). ¹H NMR (500 MHz, DMSO-d₆) δ 7.9-8.0 (m, 1H), 7.9-7.9 (m, 1H), 7.6-7.7 (m, 1H), 7.4-7.5 (m, 4H), 7.3-7.4 (m, 2H), 6.3-6.4 (m, 2H), 6.0-6.3 (m, 1H), 3.4-3.4 (m, 2H), 2.9-3.0 (m, 4H), 2.7-2.8 (m, 3H), 2.2-2.3 (m, 2H), 1.8-1.9 (m, 1H), 1.7-1.8 (m, 1H), 1.5-1.7 (m, 1H), 1.4-1.5 (m, 1H); LCMS: [M+H]⁺=481.45.

### Example 79: 6-(2-amino-6-fluoro-5-(4-(1-isopropylpiperidin-4-yl)phenyl)pyridin-3-yl)-3,4-dihydroisoquinolin-1(2H)-one (I-79)

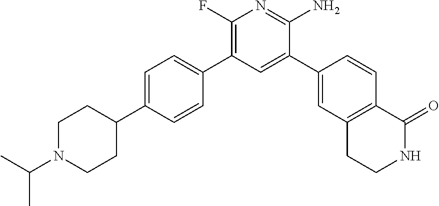 [SMILES: CC(C)N1CCC(c2ccc(-c3cc(-c4ccc5c(c4)CCNC5=O)c(N)nc3F)cc2)CC1]

A procedure analogous to that of Example 43, Step 5 using 4-(4-bromophenyl)-1-isopropylpiperidine (40 mg, 0.135 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (67.1 mg, 0.175 mmol) afforded the title compound as a beige solid (25.5 mg, 39%). ¹H NMR (500 MHz, DMSO-d₆) δ 7.98-7.93 (m, 1H), 7.93-7.88 (m, 1H), 7.67-7.61 (m, 1H), 7.52-7.44 (m, 4H), 7.29 (br d, J=8.1 Hz, 2H), 6.28 (br s, 2H), 3.45-3.39 (m, 2H), 3.01-2.93 (m, 2H), 2.92-2.85 (m, 2H), 2.77-2.68 (m, 1H), 2.49-2.43 (m, 1H), 2.23-2.22 (m, 1H), 2.22 (br t, J=11.1 Hz, 1H), 1.77 (br d, J=11.7 Hz, 2H), 1.70-1.56 (m, 2H), 1.08-0.92 (m, 6H); LCMS: [M+H]⁺=459.52.

### Example 80: 6-(2-amino-6-fluoro-5-(4-(1-isopropylpiperidin-3-yl)phenyl)pyridin-3-yl)-3,4-dihydroisoquinolin-1(2H)-one (I-80)

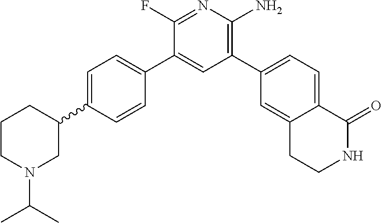 [SMILES: CC(C)N1CCCC(c2ccc(-c3cc(-c4ccc5c(c4)CCNC5=O)c(N)nc3F)cc2)C1]

A slurry of 6-(2-amino-6-fluoro-5-(4-(piperidin-3-yl)phenyl)pyridin-3-yl)-3,4-dihydroisoquinolin-1(2H)-one (40 mg, 0.096 mmol, from Example 78, Step 1) and 2-iodopropane (0.029 mL, 0.288 mmol was heated at 70° C. for 1 h. The reaction mixture was partitioned between water (3 mL) and CH₂Cl₂ (3 mL). The layers were separated and the aqueous layer was further extracted with CH₂Cl₂ (x2). The combined organic extracts were dried and concentrated onto Celite®. Reverse phase chromatography (C18; 0-50% acetonitrile/water) afforded the title compound as a beige solid (15.5 mg, 35%). ¹H NMR (500 MHz, DMSO-d₆) δ 8.00-7.94 (m, 1H), 7.94-7.88 (m, 1H), 7.66-7.62 (m, 1H), 7.60-7.52 (m, 2H), 7.50-7.46 (m, 1H), 7.46-7.43 (m, 1H), 7.43-7.35 (m, 2H), 6.46-6.21 (m, 2H), 3.57-3.49 (m, 1H), 3.46-3.36 (m, 4H), 3.21-3.11 (m, 1H), 3.09-2.99 (m, 2H), 2.98-2.93 (m, 2H), 2.06-1.97 (m, 1H), 1.95-1.89 (m, 1H), 1.88-1.69 (m, 2H), 1.33-1.24 (m, 6H). LCMS: [M+H]⁺=459.58.

### Example 81: 6-(2-amino-6-fluoro-5-(4-(1-isopropyl-1,2,3,6-tetrahydropyridin-4-yl)phenyl)pyridin-3-yl)-3,4-dihydroisoquinolin-1(2H)-one (I-81)

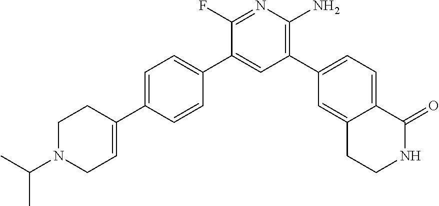 [SMILES: CC(C)N1CC=C(c2ccc(-c3cc(-c4ccc5c(c4)CCNC5=O)c(N)nc3F)cc2)CC1]

A slurry of 6-(2-amino-6-fluoro-5-(4-(1,2,3,6-tetrahydropyridin-4-yl)phenyl)pyridin-3-yl)-3,4-dihydroisoquinolin-1(2H)-one (30 mg, 0.072 mmol) and 2-iodopropane (0.029 ml, 0.290 mmol) and Et₃N (0.101 ml, 0.724 mmol) in DMF (2 mL) was heated at 70° C. for 1 h. The reaction mixture was partitioned between water (3 mL) and CH₂Cl₂ (3 mL). The layers were separated and the aqueous layer was further extracted with CH₂Cl₂ (x2). The combined organic extracts were dried and concentrated onto Celite®. Reverse phase chromatography (C18; 0-50% acetonitrile/water) afforded the title compound as a beige solid (6.5 mg, 19%). ¹H NMR (500 MHz, DMSO-d₆) δ 7.98-7.94 (m, 1H), 7.91 (d, J=7.9 Hz, 1H), 7.70-7.64 (m, 1H), 7.55-7.51 (m, 2H), 7.50-7.45 (m, 4H), 6.37-6.28 (m, 2H), 6.26-6.19 (m, 1H), 3.44-3.40 (m, 2H), 3.23-3.14 (m, 2H), 2.99-2.94 (m, 1H), 2.94-2.94 (m, 1H), 2.82-2.74 (m, 1H), 2.72-2.64 (m, 2H), 2.49-2.44 (m, 2H), 1.09-1.01 (m, 6H). LCMS: [M+H]⁺=457.45.

### Example 82: 6-(2-amino-6-fluoro-5-(4-(1-(2-hydroxyethyl)piperidin-3-yl)phenyl)pyridin-3-yl)-3,4-dihydroisoquinolin-1(2H)-one (I-82)

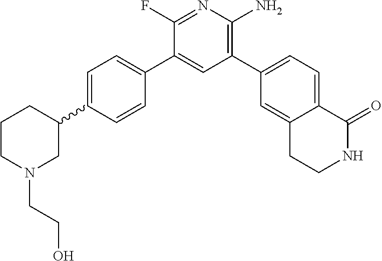 [SMILES: Nc1nc(F)c(-c2ccc(C3CCCN(CCO)C3)cc2)cc1-c1ccc2c(c1)CCNC2=O]

A procedure analogous to Example 53, Step 1 using 6-(2-amino-6-fluoro-5-(4-(piperidin-3-yl)phenyl)pyridin-3-yl)-3,4-dihydroisoquinolin-1(2H)-one (6-(2-amino-6-fluoro-5-(4-(piperidin-3-yl)phenyl)pyridin-3-yl)-3,4-dihydroisoquinolin-1(2H)-one (28 mg, 0.061 mmol) and 2-bromoethanol, 95% (22.68 mg, 0.182 mmol) at 70° C. for 1 h. The reaction mixture was partitioned between water (3 mL) and CH₂Cl₂ (3 mL). The layers were separated and the aqueous layer was further extracted with CH₂Cl₂ (x2). The combined organic extracts were dried over Na₂SO₄ and concentrated onto Celite®. Reverse phase chromatography (C18; 0-30% acetonitrile/water) afforded the title compound as a beige solid (17.5 mg, 60%). ¹H NMR (500 MHz, DMSO-d₆) δ 8.06-7.99 (m, 1H), 7.98-7.95 (m, 1H), 7.71-7.67 (m, 1H), 7.55-7.49 (m, 4H), 7.39-7.33 (m, 2H), 6.47-6.31 (m, 2H), 4.47-4.37 (m, 1H), 3.60-3.53 (m, 2H), 3.50-3.45 (m, 2H), 3.04-2.93 (m, 4H), 2.85-2.78 (m, 1H), 2.51-2.43 (m, 2H), 2.15-2.02 (m, 2H), 1.92-1.84 (m, 1H), 1.80-1.74 (m, 1H), 1.71-1.62 (m, 1H), 1.54-1.45 (m, 1H); LCMS: [M+H]⁺=461.46.

### Example 83: tert-butyl 4-((4-(6-amino-2-fluoro-5-(1-oxo-1,2,3,4-tetrahydroisoquinolin-6-yl)pyridin-3-yl)phenyl)sulfonyl)piperidine-1-carboxylate (I-83)

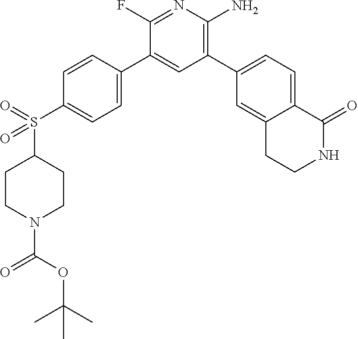 [SMILES: CC(C)(C)OC(=O)N1CCC(S(=O)(=O)c2ccc(-c3cc(-c4ccc5c(c4)CCNC5=O)c(N)nc3F)cc2)CC1]

A procedure analogous to Example 43, Step 5 using 6-(2-amino-5-bromo-6-fluoropyridin-3-yl)-3,4-dihydroisoquinolin-1(2H)-one (125 mg, 0.326 mmol) and 1-Boc-4-(4-bromobenzenesulfonyl)piperidine (110 mg, 0.272 mmol) [1,12-bis(diphenylphosphino)ferrocene]dichloropalladium(II) (19.89 mg, 0.027 mmol), and aqueous K₃PO₄ (0.418 mL of a 1.3 M solution, 0.544 mmol) afforded the title compound (91 mg, 52%) as a tan solid. ¹H NMR (500 MHz, DMSO-d₆) δ 7.95 (br s, 1H), 7.92 (d, J=7.8 Hz, 1H), 7.89-7.83 (m, 4H), 7.78 (d, J=10.1 Hz, 1H), 7.48 (dd, J=8.0, 1.7 Hz, 1H), 7.46 (s, 1H), 6.57 (s, 2H), 4.00 (br d, J=10.0 Hz, 2H), 3.50 (tt, J=11.9, 3.5 Hz, 1H), 3.41 (td, J=6.4, 2.6 Hz, 2H), 2.95 (br t, J=6.6 Hz, 2H), 2.72 (br d, J=4.5 Hz, 2H), 1.85 (br d, J=11.0 Hz, 2H), 1.36 (s, 9H), 1.35-1.30 (m, 2H); LCMS: [M−1]−=579.16, [M−100+H]⁺=481.35.

### Example 84: 6-(2-amino-5-(4-(azetidin-3-ylsulfonyl)phenyl)-6-fluoropyridin-3-yl)-3,4-dihydroisoquinolin-1(2H)-one (I-84)

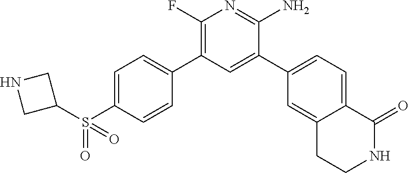 [SMILES: Nc1nc(F)c(-c2ccc(S(=O)(=O)C3CNC3)cc2)cc1-c1ccc2c(c1)CCNC2=O]

A procedure analogous to Example 43, Step 5 using 6-(2-amino-5-bromo-6-fluoropyridin-3-yl)-3,4-dihydroisoquinolin-1(2H)-one (105 mg, 0.274 mmol) and 3-[(4-bromobenzene)sulfonyl]azetidine (63.1 mg, 0.228 mmol) [1,12-bis(diphenylphosphino)ferrocene]dichloropalladium(II) (16.71 mg, 0.023 mmol), aqueous K₃PO₄ (0.351 mL of a 1.3 M solution, 0.457 mmol), DME (4 mL) and water (2 mL) to provide the title compound (37 mg, 36%) as a tan solid. ¹H NMR (500 MHz, DMSO-d₆) δ 7.95 (br s, 1H), 7.91 (d, J=7.9 Hz, 1H), 7.90-7.87 (m, 2H), 7.87-7.83 (m, 2H), 7.77 (d, J=10.1 Hz, 1H), 7.48 (dd, J=8.0, 1.5 Hz, 1H), 7.46 (s, 1H), 6.56 (br s, 2H), 4.57-4.49 (m, 1H), 3.77 (dd, J=8.6, 7.0 Hz, 2H), 3.54 (t, J=8.6 Hz, 2H), 3.41 (td, J=6.5, 2.7 Hz, 2H), 3.32 (br s, 1H), 2.95 (br t, J=6.5 Hz, 2H); LCMS: [M+H]⁺=453.14.

### Example 85: 6-(2-amino-5-(4-(ethylsulfonimidoyl)phenyl)-6-fluoropyridin-3-yl)-3,4-dihydroisoquinolin-1(2H)-one (I-85)

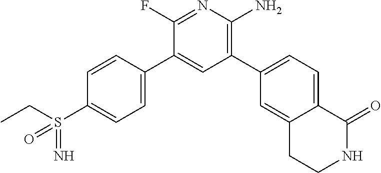 [SMILES: CCS(=N)(=O)c1ccc(-c2cc(-c3ccc4c(c3)CCNC4=O)c(N)nc2F)cc1]

A procedure analogous to Example 43, Step 5 using 6-(2-amino-5-bromo-6-fluoropyridin-3-yl)-3,4-dihydroisoquinolin-1(2H)-one (133 mg, 0.347 mmol) and 1-bromo-4-(ethylsulfonimidoyl)benzene (71.8 mg, 0.289 mmol), [1,12-bis(diphenylphosphino)ferrocene]dichloropalladium(II) (21.16 mg, 0.029 mmol), aqueous K₃PO₄ (0.445 mL of a 1.3 M solution, 0.578 mmol), DME (4 mL) and water (2 mL) afforded the title compound (57 mg, 46%) as a tan solid. ¹H NMR (500 MHz, DMSO-d₆) δ 7.95 (br s, 1H), 7.91 (d, J=8.1 Hz, 1H), 7.89 (d, J=8.4 Hz, 2H), 7.79 (br d, J=7.9 Hz, 2H), 7.75 (d, J=10.0 Hz, 1H), 7.48 (br d, J=8.1 Hz, 1H), 7.46 (s, 1H), 6.51 (br s, 2H), 4.18 (s, 1H), 3.41 (br dd, J=6.1, 4.0 Hz, 2H), 3.14 (q, J=7.3 Hz, 2H), 2.95 (br t, J=6.4 Hz, 2H), 1.08 (t, J=7.3 Hz, 3H); LCMS: [M+1]⁺=426.12.

### Example 86: 6-(2-amino-6-fluoro-5-(4-(S-methylsulfonimidoyl)phenyl)pyridin-3-yl)-3,4-dihydroisoquinolin-1(2H)-one (I-86)

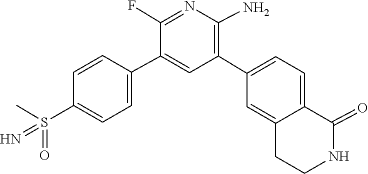 [SMILES: CS(=N)(=O)c1ccc(-c2cc(-c3ccc4c(c3)CCNC4=O)c(N)nc2F)cc1]

A procedure analogous to Example 43, Step 5 using 6-(2-amino-5-bromo-6-fluoropyridin-3-yl)-3,4-dihydroisoquinolin-1(2H)-one (126 mg, 0.329 mmol) and (4-bromophenyl)(imino)methyl-lambda6-sulfanone (64 mg, 0.274 mmol), [1,12-bis(diphenylphosphino)ferrocene]dichloropalladium(II) (20.05 mg, 0.027 mmol), K₃PO₄ (0.422 mL of a 1.3 M aqueous solution, 0.548 mmol), DME (4 mL) and water (2 mL) to provide the title compound (59 mg, 50%) as a tan solid. ¹H NMR (500 MHz, DMSO-d₆) δ 7.94 (br d, J=8.2 Hz, 3H), 7.91 (br d, J=7.9 Hz, 1H), 7.79 (br d, J=7.9 Hz, 2H), 7.74 (br d, J=10.0 Hz, 1H), 7.48 (br d, J=8.1 Hz, 1H), 7.46 (s, 1H), 6.50 (br s, 2H), 4.21 (s, 1H), 3.41 (br s, 2H), 3.32 (s, 2H), 3.08 (s, 3H), 2.96 (br t, J=6.1 Hz, 2H); LCMS: [M+H]⁺=411.15.

### Example 87: 6-(2-amino-6-fluoro-5-(4-((trifluoromethyl)sulfonyl)phenyl)pyridin-3-yl)-3,4-dihydroisoquinolin-1(2H)-one (I-87)

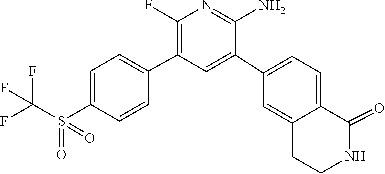 [SMILES: Nc1nc(F)c(-c2ccc(S(=O)(=O)C(F)(F)F)cc2)cc1-c1ccc2c(c1)CCNC2=O]

A procedure analogous to Example 43, Step 5 using 6-(2-amino-5-bromo-6-fluoropyridin-3-yl)-3,4-dihydroisoquinolin-1(2H)-one (107 mg, 0.279 mmol), 1-bromo-4-[(trifluoromethyl)sulfonyl]benzene (67.3 mg, 0.233 mmol), [1,12-bis(diphenylphosphino)ferrocene]dichloropalladium(II) (17.02 mg, 0.023 mmol), aqueous K₃PO₄ (0.358 mL of a 1.3 M solution, 0.465 mmol), DME (4 mL) and water (2 mL) afforded the title compound (27 mg, 24%) as a tan solid. ¹H NMR (500 MHz, DMSO-d6) δ 8.15-8.10 (m, 2H), 8.05 (d, J=8.3 Hz, 2H), 7.96 (br s, 1H), 7.92 (d, J=7.9 Hz, 1H), 7.86 (d, J=10.1 Hz, 1H), 7.49 (d, J=8.1 Hz, 1H), 7.46 (s, 1H), 6.72 (br s, 2H), 3.41 (td, J=6.4, 2.4 Hz, 2H), 2.95 (br t, J=6.5 Hz, 2H); LCMS: [M+1]⁺=466.15.

### Example 88: 6-(2-amino-5-(4-(3,4-dimethylpiperazin-1-yl)phenyl)-6-fluoropyridin-3-yl)-3,4-dihydroisoquinolin-1(2H)-one (I-88)

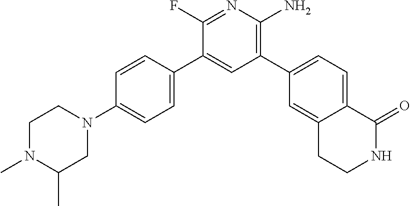 [SMILES: CC1CN(c2ccc(-c3cc(-c4ccc5c(c4)CCNC5=O)c(N)nc3F)cc2)CCN1C]

A procedure similar to Example 43, Step 5 using 6-(2-amino-5-bromo-6-fluoropyridin-3-yl)-3,4-dihydroisoquinolin-1(2H)-one (114 mg, 0.299 mmol) and 4-(4-bromophenyl)-1,2-dimethylpiperazine (67 mg, 0.249 mmol), [1,12-bis(diphenylphosphino)ferrocene]dichloropalladium(II) (18.21 mg, 0.025 mmol), K₃PO₄ (0.383 mL, 0.498 mmol), DME (4 mL) and water (2 mL) to provide the title compound (35 mg, 31%) as a tan solid. ¹H NMR (500 MHz, DMSO-d₆) δ 7.93 (br s, 1H), 7.90 (d, J=7.9 Hz, 1H), 7.58 (d, J=10.1 Hz, 1H), 7.46 (dd, J=8.0, 1.5 Hz, 1H), 7.44 (s, 1H), 7.39 (d, J=7.8 Hz, 2H), 6.97 (d, J=8.9 Hz, 2H), 6.17 (s, 2H), 3.60-3.51 (m, 2H), 3.40 (td, J=6.5, 2.7 Hz, 2H), 2.95 (t, J=6.5 Hz, 2H), 2.83-2.78 (m, 1H), 2.75 (td, J=11.5, 2.9 Hz, 1H), 2.38 (dd, J=11.6, 10.3 Hz, 1H), 2.26-2.22 (m, 1H), 2.21 (s, 3H), 2.12 (dtt, J=12.7, 6.2, 3.1 Hz, 1H), 1.05 (d, J=6.1 Hz, 3H); LCMS: [M+H]⁺=445.99.

### Example 89: 6-(2-amino-6-fluoro-5-(4-(1-(2,2,2-trifluoroethyl)piperidin-3-yl)phenyl)pyridin-3-yl)-3,4-dihydroisoquinolin-1(2H)-one (I-89)

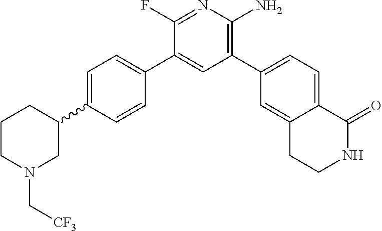 [SMILES: Nc1nc(F)c(-c2ccc(C3CCCN(CC(F)(F)F)C3)cc2)cc1-c1ccc2c(c1)CCNC2=O]

A procedure analogous to Example 53, Step 1 was employed stirring 6-(2-amino-6-fluoro-5-(4-(piperidin-3-yl)phenyl)pyridin-3-yl)-3,4-dihydroisoquinolin-1(2H)-one (40 mg, 0.086 mmol) and 2,2,2-trifluoroethyl trifluoromethanesulfonate (0.050 mL, 0.346 mmol) and Et₃N (0.120 ml, 0.864 mmol) overnight at RT. The reaction mixture was partitioned between water (3 mL) and CH₂Cl₂ (3 mL). The layers were separated and the aqueous layer was further extracted with CH₂Cl₂ (x2). The combined organic extracts were dried and concentrated onto Celite®. Silica gel chromatography (eluting with 0-70% EtOAc/Hex) afforded the title compound as a beige solid (37 mg, 78% yield based on purity of 90%). ¹H NMR (500 MHz, DMSO-d₆) δ 7.9-8.0 (m, 1H), 7.9-7.9 (m, 1H), 7.6-7.7 (m, 1H), 7.4-7.5 (m, 4H), 7.3-7.3 (m, 2H), 6.2-6.3 (m, 2H), 3.4-3.4 (m, 2H), 3.2-3.3 (m, 2H), 2.9-3.0 (m, 4H), 2.7-2.8 (m, 1H), 2.3-2.5 (m, 2H), 1.8-1.9 (m, 1H), 1.7-1.8 (m, 1H), 1.6-1.7 (m, 1H), 1.4-1.5 (m, 1H); LCMS: [M+H]⁺=499.38.

### Example 90: 6-(2-amino-6-fluoro-5-(4-(4-(2,2,2-trifluoroethyl)morpholin-2-yl)phenyl)pyridin-3-yl)-3,4-dihydroisoquinolin-1(2H)-one (I-90)

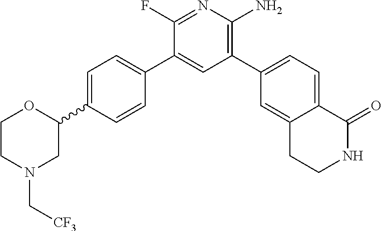 [SMILES: Nc1nc(F)c(-c2ccc(C3CN(CC(F)(F)F)CCO3)cc2)cc1-c1ccc2c(c1)CCNC2=O]

Step 1: 6-(2-amino-6-fluoro-5-(4-(4-(2,2,2-trifluoroethyl)morpholin-2-yl)phenyl)pyridin-3-yl)-3,4-dihydroisoquinolin-1(2H)-one:

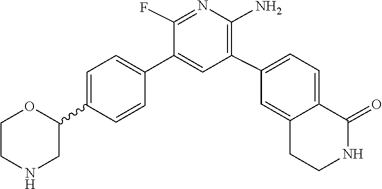 [SMILES: Nc1nc(F)c(-c2ccc(C3CNCCO3)cc2)cc1-c1ccc2c(c1)CCNC2=O]

A procedure analogous to that of Example 43, Step 5 using 2-(4-bromophenyl)morpholine (175 mg, 0.723 mmol), 6-(2-amino-5-bromo-6-fluoropyridin-3-yl)-3,4-dihydroisoquinolin-1(2H)-one (346 mg, 0.904 mmol), Cs₂CO₃ (707 mg, 2.17 mmol) and [1,1′-bis(diphenylphosphino)ferrocene]dichloropalladium(II) (68.8 mg, 0.094 mmol) afforded the product as a beige solid (259 mg, 78%). LCMS: [M+H]⁺=419.28.

Step 2: 6-(2-amino-6-fluoro-5-(4-(4-(2,2,2-trifluoroethyl)morpholin-2-yl)phenyl)pyridin-3-yl)-3,4-dihydroisoquinolin-1(2H)-one:

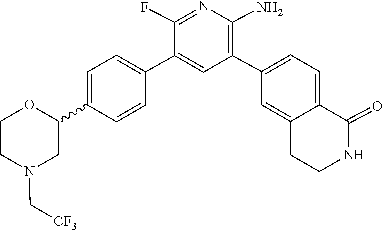 [SMILES: Nc1nc(F)c(-c2ccc(C3CN(CC(F)(F)F)CCO3)cc2)cc1-c1ccc2c(c1)CCNC2=O]

To a slurry of 6-(2-amino-6-fluoro-5-(4-(morpholin-2-yl)phenyl)pyridin-3-yl)-3,4-dihydroisoquinolin-1(2H)-one (50.mg, 0.119 mmol) in DMF (2 mL) at RT was added 2,2,2-trifluoroethyl trifluoromethanesulfonate (0.052 mL, 0.358 mmol) and Et₃N (0.167 mL, 1.20 mmol) and the mixture was stirred at 80° C. for 2.75 h. The rxn mixture was partitioned between water (3 mL) and CH₂Cl₂ (3 mL), the org phase was separated, aq phase was extracted with CH₂Cl₂ (2×3 mL), the combined organic layers were dried over Na₂SO₄ and concentrated onto Celite®. Silica gel chromatography (eluting with 0-70% EtOAc/Hex) afforded the title compound as an off-white solid (30.5 mg, 49%). ¹H NMR (500 MHz, DMSO-d₆) δ 7.9-8.0 (m, 1H), 7.9-7.9 (m, 1H), 7.6-7.7 (m, 1H), 7.5-7.6 (m, 2H), 7.5-7.5 (m, 1H), 7.4-7.5 (m, 1H), 7.4-7.4 (m, 2H), 6.3-6.4 (m, 2H), 4.5-4.6 (m, 1H), 3.9-4.0 (m, 1H), 3.7-3.8 (m, 1H), 3.4-3.5 (m, 2H), 3.2-3.3 (m, 2H), 2.9-3.0 (m, 3H), 2.85 (br d, J=11.4 Hz, 1H), 2.5-2.6 (m, 1H), 2.3-2.4 (m, 1H); LCMS: [M+H]⁺=501.38.

### Example 91: 6-(2-amino-6-fluoro-5-(4-((3aR,6aS)-tetrahydro-1H-furo[3,4-c]pyrrol-5(3H)-yl)phenyl)pyridin-3-yl)-3,4-dihydroisoquinolin-1(2H)-one (I-91)

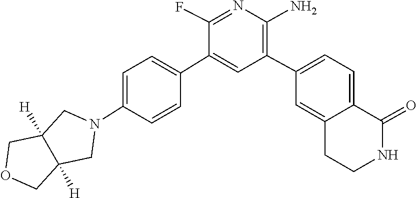 [SMILES: Nc1nc(F)c(-c2ccc(N3C[C@H]4COC[C@H]4C3)cc2)cc1-c1ccc2c(c1)CCNC2=O]

Step 1. (3aR,6aS)-5-(4-iodophenyl)hexahydro-1H-furo[3,4-c]pyrrole

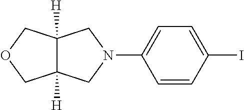 [SMILES: Ic1ccc(N2C[C@H]3COC[C@H]3C2)cc1]

A sealed mixture of hexahydro-1 H-furo[3,4-c]pyrrole·HCl (0.564 g, 3.77 mmol) and K₃PO₄ (1.60 g, 7.54 mmol) in DMF (20 mL) was stirred at RT overnight. The mixture was then degassed with a stream of N₂ and then Cul (0.144 g, 0.754 mmol), (R)-(+)-1,1′-bi-2-naphthol (0.216 g, 0.754 mmol) and 1,4-diiodobenzene (1.244 g, 3.77 mmol) were added as solids. Then degassing step was repeated. The mixture was then stirred sealed at 40° C. for 2 d. The reaction mixture was cooled to RT, diluted with EtOAc, filtered through Celite, concentrated under reduced pressure and purified by flash chromatography (50 g SiO₂, using CH₂Cl₂) to afford the product as a yellow solid (659 mg, 50% yield based on purity of 90%). LCMS: [M+H]⁺=315.99.

Step 2: 6-(2-amino-6-fluoro-5-(4-((3aR,6aS)-tetrahydro-1H-furo[3,4-c]pyrrol-5(3H)-yl)phenyl)pyridin-3-yl)-3,4-dihydroisoquinolin-1(2H)-one

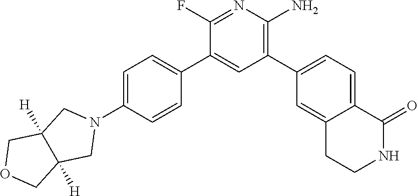 [SMILES: Nc1nc(F)c(-c2ccc(N3C[C@H]4COC[C@H]4C3)cc2)cc1-c1ccc2c(c1)CCNC2=O]

A procedure analogous to Example 1, General Method A using (3aR,6aS)-5-(4-iodophenyl)hexahydro-1H-furo[3,4-c]pyrrole (73.1 mg, 0.209 mmol, 90%), PdCl₂dppf (15.3 mg, 0.021 mmol), 6-(2-amino-5-bromo-6-fluoropyridin-3-yl)-3,4-dihydroisoquinolin-1(2H)-one (80 mg, 0.21 mmol), Cs₂CO₃ (204 mg, 0.626 mmol), H₂O (2 mL) and DME (4 mL) afforded the title compound as a white solid (7.0 mg, 7% yield based on purity of 95%). LCMS: [M+H]⁺=445.16. ¹H NMR (500 MHz, CDCl₃-CD₃OD) δ ppm 7.99 (br d, J=8.0 Hz, 1H), 7.50 (br d, J=9.7 Hz, 1H), 7.38-7.41 (m, 1H), 7.24-7.36 (m, 3H), 6.63 (br d, J=8.6 Hz, 2H), 3.89-4.05 (m, 2H), 3.57-3.72 (m, 2H), 3.51 (br t, J=6.5 Hz, 2H), 3.34-3.45 (m, 2H), 3.18-3.22 (m, 2H), 2.96-3.08 (m, 4H); LCMS: [M+H]⁺=445.16.

### Example 92: 6-(2-amino-6-fluoro-5-(4-((3aS,6aS)-hexahydro-5H-furo[2,3-c]pyrrol-5-yl)phenyl)pyridin-3-yl)-3,4-dihydroisoquinolin-1(2H)-one (I-92)

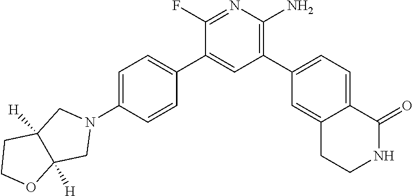 [SMILES: Nc1nc(F)c(-c2ccc(N3C[C@@H]4CCO[C@@H]4C3)cc2)cc1-c1ccc2c(c1)CCNC2=O]

Step 1: (3aS,6aS)-5-(4-iodophenyl)hexahydro-2H-furo[2,3-c]pyrrole

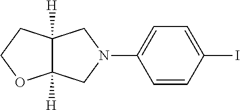 [SMILES: Ic1ccc(N2C[C@@H]3CCO[C@@H]3C2)cc1]

A sealed mixture of 1,4-diiodobenzene (2.0 g, 6.1 mmol), (3aS,6aS)-hexahydro-2H-furo[2,3-c]pyrrole·HCl (0.907 g, 6.06 mmol) and K₃PO₄ (2.57 g, 12.1 mmol) in DMF (20 mL) was stirred at rt overnight. The mixture was then degassed with a stream of N₂ and then Cul (0.231 g, 1.21 mmol) and a (R)-(+)-1,1′-bi-2-naphthol (0.347 g, 1.21 mmol) were added as solids. Then degassing step was repeated. The mixture was then stirred sealed at RT for 30 min and then at 40° C. for 4 d. Later, the reaction mixture was concentrated under reduced pressure and purified by flash chromatography (50 g SiO₂, using MeOH in CH₂Cl₂) to afford the product as a yellow-grey solid (940 mg, 48% yield based on purity of 97%). LCMS: [M+H]⁺=316.14.

Step 2. 6-(2-amino-6-fluoro-5-(4-((3aS,6aS)-hexahydro-5H-furo[2,3-c]pyrrol-5-yl)phenyl)pyridin-3-yl)-3,4-dihydroisoquinolin-1(2H)-one

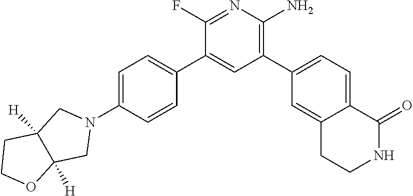 [SMILES: Nc1nc(F)c(-c2ccc(N3C[C@@H]4CCO[C@@H]4C3)cc2)cc1-c1ccc2c(c1)CCNC2=O]

A procedure analogous to Example 1, General Method A using (3aS,6aS)-5-(4-iodophenyl)hexahydro-2H-furo[2,3-c]pyrrole (67.8 mg, 0.209 mmol, 97%)PdCl₂dppf (15.27 mg, 0.021 mmol), 6-(2-amino-5-bromo-6-fluoropyridin-3-yl)-3,4-dihydroisoquinolin-1(2H)-one (80 mg, 0.21 mmol), Cs₂CO₃ (204 mg, 0.626 mmol), H₂O (2 mL) and DME (4 mL) afforded the product as a tan solid (7 mg, 7% yield based on purity of 99%). ¹H NMR (500 MHz, CDCl3) δ 8.10 (d, J=8.0 Hz, 1H), 7.50 (d, J=9.7 Hz, 1H), 7.41 (br d, J=8.0 Hz, 1H), 7.34 (br d, J=7.7 Hz, 2H), 7.27 (s, 1H), 6.54-6.65 (m, 2H), 6.01 (br s, 1H), 4.62 (t, J=5.8 Hz, 1H), 4.58 (s, 1H), 3.87-3.94 (m, 1H), 3.74-3.83 (m, 1H), 3.53-3.58 (m, 2H), 3.48-3.53 (m, 1H), 3.34-3.40 (m, 1H), 3.28-3.33 (m, 1H), 3.15-3.22 (m, 1H), 2.93-3.03 (m, 3H), 2.07-2.17 (m, 1H), 1.78-1.86 (m, 1H); LCMS [M+H]⁺=445.31.

### Example 93: 6-(2-amino-6-fluoro-5-(4-(1-(2,2,2-trifluoroethyl)piperidin-2-yl)phenyl)pyridin-3-yl)-3,4-dihydroisoquinolin-1(2H)-one (I-93)

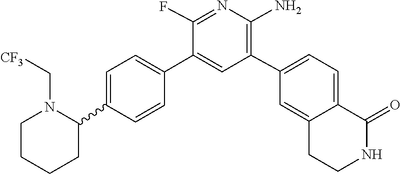 [SMILES: Nc1nc(F)c(-c2ccc(C3CCCCN3CC(F)(F)F)cc2)cc1-c1ccc2c(c1)CCNC2=O]

Step 1: 6-(2-amino-6-fluoro-5-(4-(piperidin-3-yl)phenyl)pyridin-3-yl)-3,4-dihydroisoquinolin-1(2H)-one

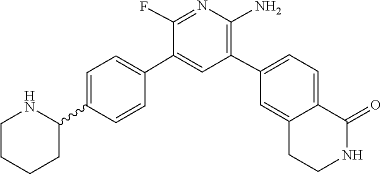 [SMILES: Nc1nc(F)c(-c2ccc(C3CCCCN3)cc2)cc1-c1ccc2c(c1)CCNC2=O]

A procedure analogous to Example 43, Step 5 using 3-(4-bromo-phenyl)-piperidine hydrochloride (175 mg, 0.633 mmol), 6-(2-amino-5-bromo-6-fluoropyridin-3-yl)-3,4-dihydroisoquinolin-1(2H)-one (303 mg, 0.791 mmol), Cs₂CO₃ (825 mg, 2.53 mmol) and [1,1′-bis(diphenylphosphino)ferrocene]dichloropalladium(II) (60.2 mg, 0.082 mmol) in DME (10.5 mL) afforded the product (192 mg, 66% yield) as a beige solid. LCMS: [M+H]⁺=417.46.

Step 2: 6-(2-amino-6-fluoro-5-(4-(1-(2,2,2-trifluoroethyl)piperidin-2-yl)phenyl)pyridin-3-yl)-3,4-dihydroisoquinolin-1(2H)-one

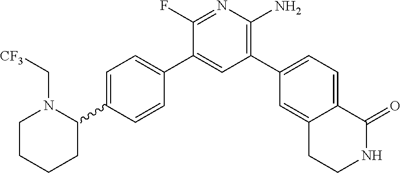 [SMILES: Nc1nc(F)c(-c2ccc(C3CCCCN3CC(F)(F)F)cc2)cc1-c1ccc2c(c1)CCNC2=O]

To a slurry of 6-(2-amino-6-fluoro-5-(4-(piperidin-2-yl)phenyl)pyridin-3-yl)-3,4-dihydroisoquinolin-1(2H)-one (45.mg, 0.089 mmol) in DMF (2 mL) at RT, was added 2,2,2-trifluoroethyl trifluoromethanesulfonate (0.026 mL, 0.177 mmol) and Et₃N (0.123 mL, 0.886 mmol) and the reaction mixture was stirred at RT for 4 h. After 4 h, additional 2,2,2-trifluoroethyl trifluoromethanesulfonate (0.026 mL, 0.177 mmol) was added, and the mixture was heated at 80° C. for 10 h. The reaction mixture was partitioned between water (3 mL) and CH₂Cl₂ (3 mL), the organic phase was separated, the aqueous phase was extracted with CH₂Cl₂ (2×3 mL), the combined layers were dried over Na₂SO₄ and concentrated onto Celite®. Purification using silica gel chromatography eluting with hexanes containing 0-80 % EtOAc, followed by reverse phase chromatography (5.5 g column, eluting with water containing 0-70% CH₃CN) afforded the title compound as an off white solid (9.5 mg, 20%). ¹H NMR (500 MHz, DMSO-d₆) δ 7.98-7.93 (m, 1H), 7.93-7.88 (m, 1H), 7.71-7.65 (m, 1H), 7.59-7.52 (m, 2H), 7.51-7.48 (m, 1H), 7.48-7.45 (m, 1H), 7.37 (br d, J=8.1 Hz, 2H), 6.40-6.26 (m, 2H), 3.44-3.37 (m, 3H), 3.23-3.17 (m, 1H), 3.09-3.00 (m, 1H), 2.99-2.94 (m, 2H), 2.88-2.78 (m, 1H), 2.49-2.42 (m, 1H), 1.79-1.72 (m, 1H), 1.72-1.64 (m, 2H), 1.64-1.50 (m, 2H), 1.43-1.33 (m, 1H). LCMS: [M+H]⁺=499.50.

### Example 94: (S)-6-(2-amino-6-fluoro-5-(4-(piperidin-3-yloxy)phenyl)pyridin-3-yl)-3,4-dihydroisoquinolin-1(2H)-one (I-94)

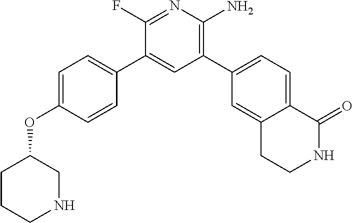 [SMILES: Nc1nc(F)c(-c2ccc(O[C@H]3CCCNC3)cc2)cc1-c1ccc2c(c1)CCNC2=O]

Step 1: tert-butyl (S)-3-(4-bromophenoxy)piperidine-1-carboxylate

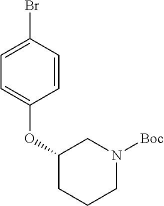 [SMILES: CC(C)(C)OC(=O)N1CCC[C@H](Oc2ccc(Br)cc2)C1]

To a suspension of NaH, (60% in mineral oil, 2.3 g, 57.5 mmol) in DMF (20 mL) was added (S)-1-Boc-3-hydroxypiperidine (2.4 g, 11.92 mmol) and 1-bromo-4-fluorobenzene (5.24 ml, 47.7 mmol) at RT. The mixture was stirred at 100° C. for overnight and then evaporated to dryness. The residue is taken up in 50 ml of ice-water and extracted with DCM. The organic extracts were dried over MgSO₄ and after filtration, the solvents were evaporated to dryness, to give the crude material. The crude material was dry loaded onto Celite® and purified by flash chromatography (0-30%, Hexanes/EtOAc) to afford the product (2.51 g, 59%) as a yellow oil. LCMS: [M-Me+H]⁺=341.26.

Step 2: tert-butyl (S)-3-(4-(6-amino-2-fluoro-5-(1-oxo-1,2,3,4-tetrahydroisoquinolin-6-yl)pyridin-3-yl)phenoxy)piperidine-1-carboxylate

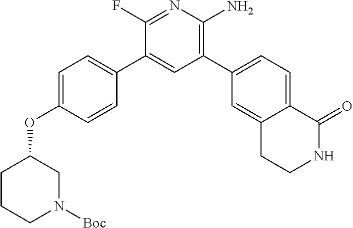 [SMILES: CC(C)(C)OC(=O)N1CCC[C@H](Oc2ccc(-c3cc(-c4ccc5c(c4)CCNC5=O)c(N)nc3F)cc2)C1]

To a solution of tert-butyl (S)-3-(4-bromophenoxy)piperidine-1-carboxylate (155 mg, 0.434 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (128 mg, 0.334 mmol) in 1,4-dioxane (3 mL) was added a solution of K₃PO₄ (213 mg, 1.00 mmol) in water (1 mL) at RT and the reaction mixture was degassed under argon for 15 minutes. Then XPhos Pd G2 (26.3 mg, 0.033 mmol) was added and the reaction mixture was heated at 90° C. for overnight. The reaction mixture was dry loaded onto Celite® and purified by flash chromatography (0-20%, DCM/MeOH) to afford the product (46.9 mg, 26%) as a beige powder. LCMS: [M+H]⁺=533.34.

Step 3: (S)-6-(2-amino-6-fluoro-5-(4-(piperidin-3-yloxy)phenyl)pyridin-3-yl)-3,4-dihydroisoquinolin-1(2H)-one

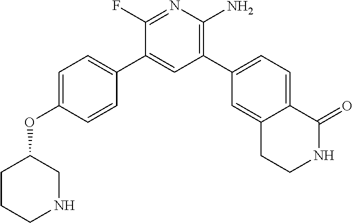 [SMILES: Nc1nc(F)c(-c2ccc(O[C@H]3CCCNC3)cc2)cc1-c1ccc2c(c1)CCNC2=O]

To a solution of tert-butyl (S)-3-(4-(6-amino-2-fluoro-5-(1-oxo-1,2,3,4-tetrahydroisoquinolin-6-yl)pyridin-3-yl)phenoxy)piperidine-1-carboxylate (46.9 mg, 0.088 mmol) in CH₂Cl₂ (4 mL) was added TFA (2 mL, 26.1 mmol) at RT and the reaction mixture was stirred for 30 min. The solvents were removed and the crude mixture was dissolved in MeOH and passed through a PoraPak Rxn CX (15cc-2g) cartridge in a catch & elute method. The cartridge was washed with MeOH (20 mL), then the solution of product in MeOH was added onto the cartridge and the cartridge was rinse with MeOH (3×20 mL). The product was released using 20 mL of a solution of ammonia in MeOH (3N) to afford the free base of the crude material which was dry loaded and purified using flash chromatography (0-30%, MeOH/DCM) to afford the title compound (25.8 mg, 64%) as a beige powder. ¹H NMR (500 MHz, DMSO-d₆) δ 7.94 (br s, 1H), 7.90 (d, J=7.9 Hz, 1H), 7.60 (d, J=10.1 Hz, 1H), 7.50-7.43 (m, 4H), 7.01 (d, J=8.8 Hz, 2H), 6.23 (s, 2H), 4.45-4.36 (m, 1H), 3.43-3.38 (m, 2H), 3.21-3.13 (m, 2H), 2.99-2.92 (m, 2H), 2.89-2.81 (m, 1H), 2.73-2.60 (m, 2H), 2.07-1.96 (m, 1H), 1.78-1.69 (m, 1H), 1.64-1.46 (m, 2H); LCMS: [M+H]⁺=433.42.

### Example 95: 6-(2-amino-6-fluoro-5-(4-(4-isopropylpiperazin-1-yl)phenyl)pyridin-3-yl)-7-fluoro-3,4-dihydroisoquinolin-1(2H)-one (I-95)

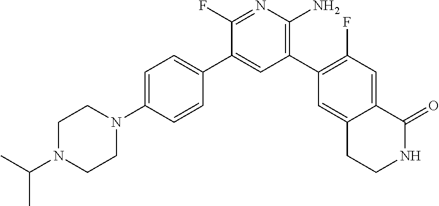 [SMILES: CC(C)N1CCN(c2ccc(-c3cc(-c4cc5c(cc4F)C(=O)NCC5)c(N)nc3F)cc2)CC1]

Step 1: 8-fluoro-6-(4,4, 5,5-tetramethyl-1,3,2-dioxaborolan-2-yl)-3,4-dihydroiso-quinolin-1(2H)-one

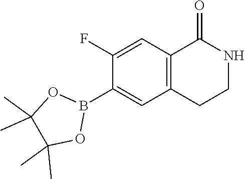 [SMILES: CC1(C)OB(c2cc3c(cc2F)C(=O)NCC3)OC1(C)C]

To 6-bromo-5-fluoro-3,4-dihydroisoquinolin-1(2H)-one (50 mg, 0.205 mmol, 1eq), bis(pinacolato)diboron (58.2 mg, 0.229 mmol, 1.1 eq), KOAc (61.3 mg, 0.625 mmol, 3 eq) and [1,12-bis(diphenylphosphino)ferrocene]dichloropalladium(II) (7.62 mg, 10.41 μmol, 0.05 eq) in 1,4-dioxane (2.5 mL) was added under Ar. The mixture was heated in a microwave for 2 h at 100° C. The LCMS showed less than 10% of starting material. The reaction mixture was used in the next step without purification. LCMS: [M+H]⁺=292.0 Step 2: 6-(2-amino-5-bromo-6-fluoropyridin-3-yl)-7-fluoro-3,4-dihydroisoquinolin-1(2H)-one

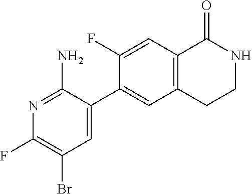 [SMILES: Nc1nc(F)c(Br)cc1-c1cc2c(cc1F)C(=O)NCC2]

A procedure similar to Example 1, General Method A was used heating a sealed, degassed mixture of H₂O (1 mL), 5-bromo-6-fluoro-3-iodopyridin-2-amine (200 mg, 0.63 mmol), 7-fluoro-6-(4,4,5,5-tetramethyl-1,3,2-dioxaborolan-2-yl)-3,4-dihydroisoquinolin-1(2H)-one (184 mg, 0.63 mmol), K₂CO₃ (183 mg, 1.32 mmol), Pd(PPh₃)₂Cl₂ (53 mg, 0.076 mmol) and DMF (9 mL) in a microwave reactor at 80° C. for 1 h. Purification by flash chromatography (eluting with MeOH in CH₂Cl₂) afforded the title compound as a tan solid (79.0 mg, 34% yield based on purity of 97%). LCMS: [M+H]⁺=354.19/356.15.

Step 2. 6-(2-amino-6-fluoro-5-(4-(4-isopropylpiperazin-1-yl)phenyl)pyridin-3-yl)-7-fluoro-3,4-dihydroisoquinolin-1(2H)-one

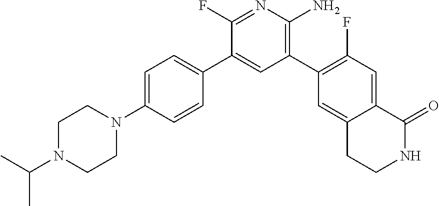 [SMILES: CC(C)N1CCN(c2ccc(-c3cc(-c4cc5c(cc4F)C(=O)NCC5)c(N)nc3F)cc2)CC1]

A procedure similar to Example 43, Step 5 heating a mixture of 4-(4-isopropylpiperazinyl)phenylboronic acid, pinacol ester (86 mg, 0.260 mmol), 6-(2-amino-5-bromo-6-fluoropyridin-3-yl)-7-fluoro-3,4-dihydroisoquinolin-1(2H)-one (79 mg, 0.22 mmol), Cs₂CO₃ (211 mg, 0.649 mmol), H₂O (2 mL) DME (4 mL) and PdCl₂dppf (15.8 mg, 0.022 mmol) under microwave irradiation at 90° C. for 1 h, afforded the title compound as a pale yellow solid (42.0 mg, 40% yield based on purity of 98%). ¹H NMR (500 MHz, DMSO-d₆) δ 8.11 (br s, 1H), 7.54-7.66 (m, 2H), 7.43 (d, J=7.1 Hz, 1H), 7.38 (br d, J=8.0 Hz, 2H), 6.96 (d, J=8.9 Hz, 2H), 6.18 (s, 2H), 3.38-3.45 (m, 2H), 3.10-3.20 (m, 4H), 2.92 (br t, J=6.4 Hz, 2H), 2.63-2.71 (m, 1H), 2.54-2.61 (m, 4H), 1.01 (d, J=6.6 Hz, 6H); LCMS: [M+H]⁺=478.42.

### Example 96: 6-(2-amino-6-fluoro-5-(4-((3R,5S)-3,4,5-trimethylpiperazin-1-yl)phenyl)pyridin-3-yl)-3,4-dihydroisoquinolin-1(2H)-one (I-96)

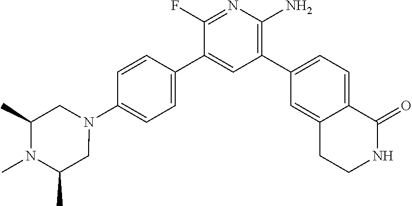 [SMILES: C[C@@H]1CN(c2ccc(-c3cc(-c4ccc5c(c4)CCNC5=O)c(N)nc3F)cc2)C[C@H](C)N1C]

Step 1: (2S,6R)-4-(4-bromophenyl)-1,2,6-trimethylpiperazine

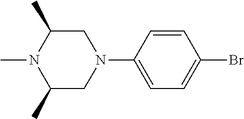 [SMILES: C[C@@H]1CN(c2ccc(Br)cc2)C[C@H](C)N1C]

To a 30 mL vial was added 1-bromo-4-iodobenzene (529 mg, 1.87 mmol), cis-1,2,6-trimethylpiperazine (288 mg, 2.24 mmol), tris(dibenzylideneacetone)dipalladium (0) (51.4 mg, 0.056 mmol), 4,5-bis(diphenylphosphino)-9,9-dimethylxanthene (97 mg, 0.168 mmol) and Cs₂CO₃ (1828 mg, 5.61 mmol). The vial was sealed with a cap and septum and then the reaction vessel was evacuated and backfilled with nitrogen. Toluene (4 mL) was added and the reaction vessel was evacuated and backfilled with nitrogen an additional time. The reaction was then concentrated onto Celite® and purified by flash chromatography (SiO₂, hexanes-EtOAc) to give the product (272 mg, 51%) as a brown solid. LCMS: [M+H]⁺=284.80.

Step 2: 6-(2-amino-6-fluoro-5-(4-((3R,5S)-3,4,5-trimethylpiperazin-1-yl)phenyl)pyridin-3-yl)-3,4-dihydroisoquinolin-1(2H)-one

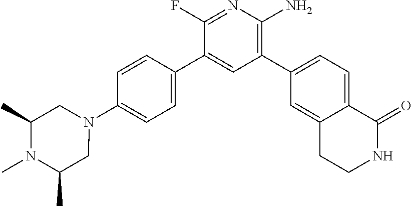 [SMILES: C[C@@H]1CN(c2ccc(-c3cc(-c4ccc5c(c4)CCNC5=O)c(N)nc3F)cc2)C[C@H](C)N1C]

A procedure similar to Example 43, Step 5 using 6-(2-amino-5-bromo-6-fluoropyridin-3-yl)-3,4-dihydroisoquinolin-1(2H)-one (162 mg, 0.424 mmol), cis-4-(4-bromophenyl)-1,2,6-trimethylpiperazine (100 mg, 0.353 mmol), [1,12-bis(diphenylphosphino)ferrocene]dichloropalladium(II) (25.8 mg, 0.035 mmol), aqueous K₃PO₄ (0.543 mL of a 1.3 M solution, 0.706 mmol), DME (4 mL) and water (2 mL) afforded the title compound (50 mg, 29%) as a tan solid. ¹H NMR (500 MHz, DMSO-d₆) δ 7.94 (br s, 1H), 7.90 (d, J=7.9 Hz, 1H), 7.58 (d, J=10.1 Hz, 1H), 7.46 (d, J=8.1 Hz, 1H), 7.44 (s, 1H), 7.39 (br d, J=8.1 Hz, 2H), 6.97 (br d, J=8.8 Hz, 2H), 6.17 (s, 2H), 3.60 (br d, J=11.0 Hz, 2H), 3.40 (td, J=6.4, 2.6 Hz, 2H), 2.95 (br t, J=6.5 Hz, 2H), 2.41 (br t, J=9.7 Hz, 2H), 2.20 (br s, 5H), 1.08 (br d, J=5.6 Hz, 6H); LCMS: [M+1]⁺=461.19.

### Example 97: 6-(2-amino-6-fluoro-5-(4-((3S,5S)-3,4,5-trimethylpiperazin-1-yl)phenyl)pyridin-3-yl)-3,4-dihydroisoquinolin-1(2H)-one (I-97)

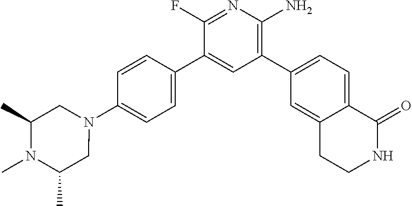 [SMILES: C[C@H]1CN(c2ccc(-c3cc(-c4ccc5c(c4)CCNC5=O)c(N)nc3F)cc2)C[C@H](C)N1C]

A procedure analogous to Example 43, Step 5 using 6-(2-amino-5-bromo-6-fluoropyridin-3-yl)-3,4-dihydroisoquinolin-1(2H)-one (119 mg, 0.309 mmol), (2S,6S)-4-(4-bromophenyl)-1,2,6-trimethylpiperazine (73 mg, 0.258 mmol), [1,12-bis(diphenylphosphino)ferrocene]dichloropalladium(II) (18.86 mg, 0.026 mmol), aqueous K₃PO₄ (0.397 mL of a 1.3 M solution, 0.516 mmol), DME (4 mL) and water (2 mL) afforded the title compound (31 mg, 25%) as a tan solid. ¹H NMR (500 MHz, DMSO-d₆) δ 7.94 (br s, 1H), 7.90 (d, J=7.8 Hz, 1H), 7.57 (d, J=10.1 Hz, 1H), 7.46 (d, J=8.1 Hz, 1H), 7.44 (s, 1H), 7.39 (br d, J=8.1 Hz, 2H), 6.96 (br d, J=8.7 Hz, 2H), 6.17 (s, 2H), 3.40 (td, J=6.5, 2.6 Hz, 2H), 3.22 (br d, J=1.7 Hz, 2H), 2.95 (br t, J=6.5 Hz, 6H), 2.29 (br s, 3H), 1.05 (br s, 6H); LCMS: [M+H]⁺=461.34.

### Example 98: 6-(2-amino-6-fluoro-5-(4-(morpholinosulfonyl)phenyl)pyridin-3-yl)-3,4-dihydroisoquinolin-1(2H)-one(I-98)

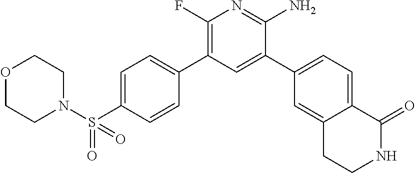 [SMILES: Nc1nc(F)c(-c2ccc(S(=O)(=O)N3CCOCC3)cc2)cc1-c1ccc2c(c1)CCNC2=O]

A procedure analogous to Example 43, Step 5 using 6-(2-amino-5-bromo-6-fluoropyridin-3-yl)-3,4-dihydroisoquinolin-1(2H)-one (100 mg, 0.297 mmol), 4-N-morpholinylsulfonylphenylboronic acid (97 mg, 0.357 mmol), [1,12-bis(diphenylphosphino)ferrocene]dichloropalladium(II) (21.77 mg, 0.030 mmol), aqueous K₃PO₄ (0.458 mL of a 1.3 M solution, 0.595 mmol), DME (4 mL) and water (2 mL) afforded the title compound (20 mg, 14%) as a tan solid. ¹H NMR (500 MHz, DMSO-d₆) δ 7.95 (br s, 1H), 7.91 (d, J=7.9 Hz, 1H), 7.86 (d, J=7.9 Hz, 2H), 7.79-7.74 (m, 3H), 7.49 (d, J=7.9 Hz, 1H), 7.47 (s, 1H), 6.55 (s, 2H), 3.67-3.61 (m, 4H), 3.41 (td, J=6.4, 2.6 Hz, 2H), 2.95 (br t, J=6.5 Hz, 2H), 2.91-2.87 (m, 4H); LCMS: [M+H]⁺=483.31.

### Example 99: 6-(2-amino-6-fluoro-5-(4-(pyrrolidin-1-ylsulfonyl)phenyl)pyridin-3-yl)-3,4-dihydroisoquinolin-1(2H)-one (I-99)

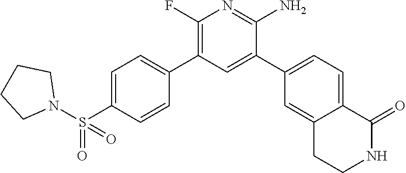 [SMILES: Nc1nc(F)c(-c2ccc(S(=O)(=O)N3CCCC3)cc2)cc1-c1ccc2c(c1)CCNC2=O]

A procedure similar to Example 43, Step 5 using 6-(2-amino-5-bromo-6-fluoropyridin-3-yl)-3,4-dihydroisoquinolin-1(2H)-one (100 mg, 0.297 mmol) and 4-(pyrrolidinylsulfonyl)phenylboronic acid (91 mg, 0.357 mmol) afforded the title compound (112 mg, 77%) as a tan solid. ¹H NMR (500 MHz, DMSO-d₆) δ 7.95 (br s, 1H), 7.91 (d, J=7.8 Hz, 1H), 7.82 (s, 4H), 7.76 (d, J=10.1 Hz, 1H), 7.50-7.47 (m, 1H), 7.46 (s, 1H), 6.53 (s, 2H), 3.41 (td, J=6.4, 2.5 Hz, 2H), 3.16 (br t, J=6.6 Hz, 4H), 2.99-2.91 (m, 2H), 1.70-1.64 (m, 4H); LCMS: [M+H]⁺=467.28.

### Example 100: 6-(2-amino-5-(4-(1-ethylpiperidin-2-yl)phenyl)-6-fluoropyridin-3-yl)-3,4-dihydroisoquinolin-1(2H)-one (I-100)

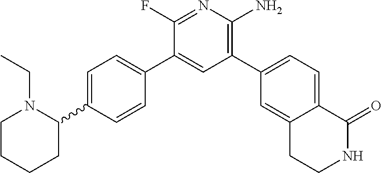 [SMILES: CCN1CCCCC1c1ccc(-c2cc(-c3ccc4c(c3)CCNC4=O)c(N)nc2F)cc1]

A procedure analogous to Example 93, Step 2 using 6-(2-amino-6-fluoro-5-(4-(piperidin-2-yl)phenyl)pyridin-3-yl)-3,4-dihydroisoquinolin-1(2H)-one (45 mg, 0.089 mmol) and bromoethane (0.013 mL, 0.177 mmol) afforded the title compound (23 mg, 56%) as a beige solid. ¹H NMR (500 MHz, DMSO-d₆) δ 8.00-7.93 (m, 1H), 7.93-7.88 (m, 1H), 7.70-7.64 (m, 1H), 7.53-7.46 (m, 4H), 7.40-7.31 (m, 2H), 6.38-6.27 (m, 2H), 3.46-3.40 (m, 2H), 3.15-3.01 (m, 2H), 3.00-2.94 (m, 2H), 2.49-2.39 (m, 1H), 2.09-1.99 (m, 1H), 1.99-1.89 (m, 1H), 1.78-1.62 (m, 3H), 1.60-1.53 (m, 1H), 1.47-1.30 (m, 2H), 0.90-0.85 (m, 3H); LCMS: [M+H]⁺=445.54

### Example 101: 6-(2-amino-5-(4-(1-(2,2-difluoroethyl)piperidin-2-yl)phenyl)-6-fluoropyridin-3-yl)-3,4-dihydroisoquinolin-1(2H)-one (I-101)

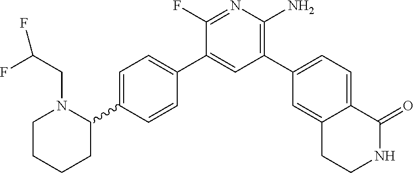 [SMILES: Nc1nc(F)c(-c2ccc(C3CCCCN3CC(F)F)cc2)cc1-c1ccc2c(c1)CCNC2=O]

A procedure similar to that of Example 93, Step 2 using 6-(2-amino-6-fluoro-5-(4-(piperidin-2-yl)phenyl)pyridin-3-yl)-3,4-dihydroisoquinolin-1(2H)-one (45 mg, 0.089 mmol) and 2,2-difluoroethyl trifluoromethanesulfonate (0.023 mL, 0.177 mmol) afforded the title compound (26.5 mg, 59%) as a beige solid. ¹H NMR (500 MHz, DMSO-d₆) δ 8.01-7.93 (m, 1H), 7.93-7.88 (m, 1H), 7.69-7.65 (m, 1H), 7.55-7.51 (m, 2H), 7.51-7.48 (m, 1H), 7.48-7.45 (m, 1H), 7.37 (d, J=8.2 Hz, 2H), 6.32 (s, 2H), 6.15-5.89 (m, 1H), 3.46-3.39 (m, 2H), 3.27-3.22 (m, 1H), 3.20-3.13 (m, 1H), 3.00-2.94 (m, 2H), 2.77-2.64 (m, 1H), 2.42-2.26 (m, 2H), 1.79-1.71 (m, 1H), 1.70-1.63 (m, 2H), 1.63-1.53 (m, 1H), 1.53-1.43 (m, 1H), 1.41-1.30 (m, 1H); LCMS: [M+H]⁺=481.45.

### Example 102: 6-(2-amino-5-(4-(4-ethylmorpholin-2-yl)phenyl)-6-fluoropyridin-3-yl)-3,4-dihydroisoquinolin-1(2H)-one (I-102)

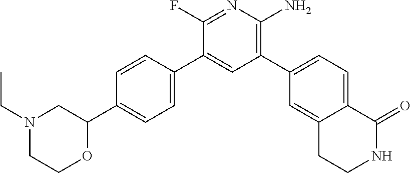 [SMILES: CCN1CCOC(c2ccc(-c3cc(-c4ccc5c(c4)CCNC5=O)c(N)nc3F)cc2)C1]

A procedure analogous to that of Example 43, Step 5 using 2-(4-bromophenyl)morpholine (175 mg, 0.723 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one(346 mg, 0.904 mmol) afforded the product as a beige solid (259 mg, 69% based on purity of 78%). LCMS: [M+H]⁺=419.28.
Step 2: 6-(2-Amino-5-(4-(4-ethylmorpholin-2-yl)phenyl)-6-fluoropyridin-3-yl)-3,4-dihydroisoquinolin-1(2H)-one

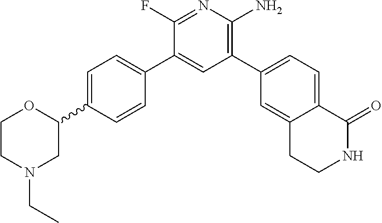 [SMILES: CCN1CCOC(c2ccc(-c3cc(-c4ccc5c(c4)CCNC5=O)c(N)nc3F)cc2)C1]

A procedure similar to Example 90, Step 2 using 6-(2-amino-6-fluoro-5-(4-(morpholin-2-yl)phenyl)pyridin-3-yl)-3,4-dihydroisoquinolin-1(2H)-one (50 mg, 78% purity, 0.093 mmol) and bromoethane (0.014 mL, 0.186 mmol) and heating at 80° C. for 3 h afforded the title compound (27.5 mg, 63%) as a beige solid. ¹H NMR (500 MHz, DMSO-d₆) δ 7.99-7.93 (m, 1H), 7.93-7.88 (m, 1H), 7.67-7.63 (m, 1H), 7.55-7.50 (m, 2H), 7.50-7.46 (m, 1H), 7.46-7.44 (m, 1H), 7.42-7.37 (m, 2H), 6.41-6.25 (m, 2H), 4.54-4.44 (m, 1H), 4.00-3.91 (m, 1H), 3.73-3.63 (m, 1H), 3.45-3.39 (m, 2H), 3.00-2.90 (m, 3H), 2.82-2.75 (m, 1H), 2.42-2.32 (m, 2H), 2.12-2.03 (m, 1H), 1.93-1.84 (m, 1H), 1.03 (t, J=7.2 Hz, 3H); LCMS: [M+H]⁺=447.54.

### Example 103: 6-(2-amino-5-(4-(4-(2,2-difluoroethyl)morpholin-2-yl)phenyl)-6-fluoropyridin-3-yl)-3,4-dihydroisoquinolin-1(2H)-one (I-103)

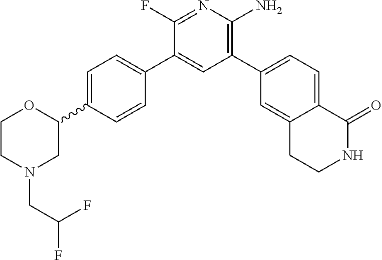 [SMILES: Nc1nc(F)c(-c2ccc(C3CN(CC(F)F)CCO3)cc2)cc1-c1ccc2c(c1)CCNC2=O]

A procedure similar to Example 90, Step 2 using 6-(2-amino-6-fluoro-5-(4-(morpholin-2-yl)phenyl)pyridin-3-yl)-3,4-dihydroisoquinolin-1(2H)-one (50 mg, 78% purity, 0.093 mmol), and 2,2-difluoroethyl trifluoromethanesulfonate (0.037 mL, 0.280 mmol) and heating at 80° C. for 3 h afforded the title compound (21.5 mg, 45%) as a beige solid. ¹H NMR (500 MHz, DMSO-d₆) δ 7.99-7.93 (m, 1H), 7.93-7.88 (m, 1H), 7.68-7.63 (m, 1H), 7.54 (br d, J=7.7 Hz, 2H), 7.50-7.47 (m, 1H), 7.47-7.44 (m, 1H), 7.42-7.36 (m, 2H), 6.42-6.31 (m, 2H), 6.31-6.07 (m, 1H), 4.63-4.44 (m, 1H), 4.02-3.89 (m, 1H), 3.76-3.64 (m, 1H), 3.47-3.39 (m, 2H), 3.03-2.93 (m, 3H), 2.87-2.74 (m, 3H), 2.44-2.36 (m, 1H), 2.19 (t, J=10.8 Hz, 1H). LCMS: [M+H]⁺=483.46.

### Example 104: 6-(2-amino-6-fluoro-5-(4-(1-isopropylpiperidin-2-yl)phenyl)pyridin-3-yl)-3,4-dihydroisoquinolin-1(2H)-one (I-104)

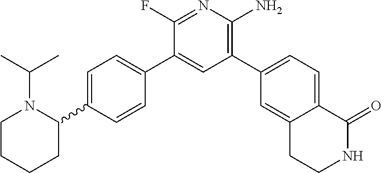 [SMILES: CC(C)N1CCCCC1c1ccc(-c2cc(-c3ccc4c(c3)CCNC4=O)c(N)nc2F)cc1]

A procedure analogous to that of Example 90, Step 2 was used by heating 6-(2-amino-6-fluoro-5-(4-(piperidin-2-yl)phenyl)pyridin-3-yl)-3,4-dihydroisoquinolin-1(2H)-one (45 mg, 0.089 mmol, purity 82%) and 2-iodopropane (0.018 ml, 0.177 mmol) overnight at 80° C. The reaction mixture was partitioned between water (3 mL) and CH₂Cl₂ (3 mL). The layers were separated and the aqueous layer was further extracted with CH₂Cl₂ (x2). The combined organic extracts was dried and concentrated onto Celite®. Reverse phase chromatography (C18; 0-40% acetonitrile/water) followed by silica gel chromatography (eluting with MeOH/CH₂Cl₂ 0-5%) afforded the title compound (11.5 mg, 27%) as a beige solid. ¹H NMR (500 MHz, DMSO-d₆) δ 8.9-9.0 (m, 1H), 7.9-8.0 (m, 1H), 7.9-7.9 (m, 1H), 7.7-7.8 (m, 3H), 7.62 (br d, J=7.3 Hz, 2H), 7.5-7.5 (m, 2H), 6.4-6.5 (m, 2H), 4.4-4.5 (m, 1H), 3.5-3.6 (m, 1H), 3.4-3.5 (m, 2H), 3.1-3.2 (m, 1H), 2.9-3.1 (m, 3H), 1.9-2.1 (m, 5H), 1.7-1.7 (m, 1H), 1.2-1.2 (m, 3H), 1.14 (br d, J=6.4 Hz, 3H). LCMS: [M+H]⁺=459.45

### Example 105: 6-(2-amino-6-fluoro-5-(4-(4-isopropylmorpholin-2-yl)phenyl)pyridin-3-yl)-3,4-dihydroisoquinolin-1(2H)-one (I-105)

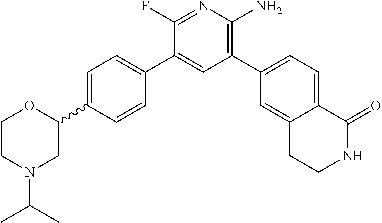 [SMILES: CC(C)N1CCOC(c2ccc(-c3cc(-c4ccc5c(c4)CCNC5=O)c(N)nc3F)cc2)C1]

A procedure analogous to that of Example 90, Step 2 using 6-(2-amino-6-fluoro-5-(4-(morpholin-2-yl)phenyl)pyridin-3-yl)-3,4-dihydroisoquinolin-1(2H)-one (50 mg, 78% purity, 0.093 mmol, from Example 102) and 2-iodopropane (0.028 mL, 0.280 mmol) afforded the title compound (11 mg, 24%) as an off white solid. ¹H NMR (500 MHz, DMSO-d₆) δ 7.9-8.0 (m, 1H), 7.9-7.9 (m, 1H), 7.6-7.7 (m, 1H), 7.5-7.5 (m, 2H), 7.5-7.5 (m, 1H), 7.4-7.5 (m, 1H), 7.4-7.4 (m, 2H), 6.2-6.5 (m, 2H), 4.4-4.6 (m, 1H), 3.9-4.1 (m, 1H), 3.6-3.7 (m, 1H), 3.4-3.4 (m, 2H), 2.9-3.0 (m, 2H), 2.8-2.9 (m, 1H), 2.6-2.7 (m, 2H), 2.3-2.4 (m, 1H), 2.1-2.1 (m, 1H), 0.99 (d, J=6.5 Hz, 6H); LCMS: [M+H]⁺=461.52.

### Example 106: 6-(2-amino-6-fluoro-5-(4-((1S,5R)-3-methyl-3-azabicyclo[3.1.0]hexan-1-yl)phenyl)pyridin-3-yl)-3,4-dihydroisoquinolin-1(2H)-one (I-106)

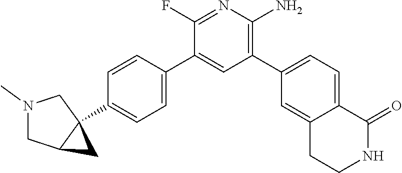 [SMILES: CN1C[C@@H]2C[C@]2(c2ccc(-c3cc(-c4ccc5c(c4)CCNC5=O)c(N)nc3F)cc2)C1]

Step 1: (1S,5R)-1-(4-bromophenyl)-3-methyl-3-azabicyclo[3.1.0]hexane

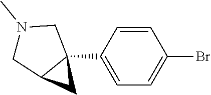

A suspension of stirred (1S,5R)-1-(4-bromophenyl)-3-azabicyclo[3.1.0]hexane (200 mg, 0.840 mmol) in anh. THF(10 mL) was treated with an HCHO solution (280 mg, 0.70 mL, 9.3 mmol, aq 37%) at rt. After 1.5 h of stirring at RT, NaBH(OAc)₃ (231 mg, 1.09 mmol) was added in one portion while cooling in an ice-H₂O bath. The cooling bath was removed after 5 min and stirring was continued at RT for 20 h. Later, excess MeOH was added, the reaction mixture was concentrated under reduced pressure and then filtered through a Waters PoraPak CX column to afford the product as a pale yellow oil (162 mg, 76%). LCMS: [M+H]⁺=252.14/254.17.

Step 2: 6-(2-amino-6-fluoro-5-(4-((1S,5R)-3-methyl-3-azabicyclo[3.1.0]hexan-1-yl)phenyl)pyridin-3-yl)-3,4-dihydroisoquinolin-1(2H)-one

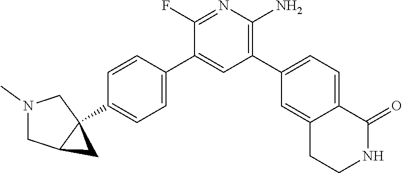 [SMILES: CN1C[C@@H]2C[C@]2(c2ccc(-c3cc(-c4ccc5c(c4)CCNC5=O)c(N)nc3F)cc2)C1]

A procedure analogous to Example 1, General Method A using (1S,5R)-1-(4-bromophenyl)-3-methyl-3-azabicyclo[3.1.0]hexane (81 mg, 0.321 mmol), 6-(2-amino-6-fluoro-5-(4,4,5,5-tetramethyl-1,3,2-dioxaborolan-2-yl)pyridin-3-yl)-3,4-dihydroisoquinolin-1(2H)-one (124 mg, 0.324 mmol), Cs₂CO₃ (314 mg, 0.964 mmol), PdCl₂dppf (23.5 mg, 0.032 mmol), DME (3 mL), H₂O (1.5 mL) afforded the title compound as an off white solid (35 mg, 24% yield based on purity of 95%). ¹H NMR (500 MHz, DMSO-d₆) δ 7.93-7.97 (m, 1H), 7.91 (d, J=7.8 Hz, 1H), 7.62 (d, J=10.0 Hz, 1H), 7.43-7.49 (m, 4H), 7.18 (d, J=8.3 Hz, 2), 6.29 (s, 2H), 3.39-3.44 (m, 2H), 3.30-3.32 (m, 1H), 3.24-3.28 (m, 1H), 2.93-3.00 (m, 3H), 2.39 (dd, J=8.6, 3.4 Hz, 1H), 2.30 (s, 3H), 1.77-1.82 (m, 1H), 1.36 (t, J=4.0 Hz, 1H), 0.77 (dd, J=7.8, 3.8 Hz, 1H); LCMS: [M+H]⁺=429.43.

### Example 107: 6-(2-amino-6-fluoro-5-(4-(1-(2-methoxyethyl)piperidin-4-yl)phenyl)pyridin-3-yl)-3,4-dihydroisoquinolin-1(2H)-one (I-107)

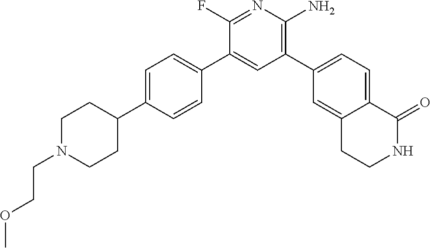 [SMILES: COCCN1CCC(c2ccc(-c3cc(-c4ccc5c(c4)CCNC5=O)c(N)nc3F)cc2)CC1]

Step 1: tert-butyl 4-(4-(6-amino-2-fluoro-5-(1-oxo-1,2,3,4-tetrahydroisoquinolin-6-yl)pyridin-3-yl)phenyl)piperidine-1-carboxylate

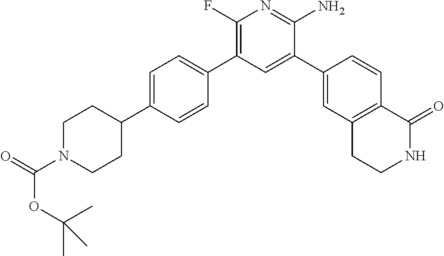 [SMILES: CC(C)(C)OC(=O)N1CCC(c2ccc(-c3cc(-c4ccc5c(c4)CCNC5=O)c(N)nc3F)cc2)CC1]

A procedure analogous to Example 43, Step 5 using 4-(N-Boc-piperidino)phenylboronic acid pinacol ester (300 mg, 0.773 mmol) and 6-(2-amino-5-bromo-6-fluoropyridin-3-yl)-3,4-dihydroisoquinolin-1(2H)-one (200 mg, 0.595 mmol) afforded the product as a beige solid (207 mg, 59% yield based on purity of 87%). LCMS: [M+H]⁺=517.49.

Step 2: 6-(2-amino-6-fluoro-5-(4-(piperidin-4-yl)phenyl)pyridin-3-yl)-3,4-dihydroisoquinolin-1(2H)-one

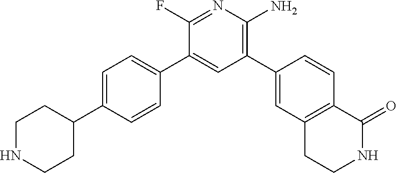 [SMILES: Nc1nc(F)c(-c2ccc(C3CCNCC3)cc2)cc1-c1ccc2c(c1)CCNC2=O]

To a solution of tert-butyl 4-(4-(6-amino-2-fluoro-5-(1-oxo-1,2,3,4-tetrahydroisoquinolin-6-yl)pyridin-3-yl)phenyl)piperidine-1-carboxylate (205 mg, 0.397 mmol)) at RT with CH₂Cl₂ (2 mL) and TFA (0.5 mL) was added. The mixture was stirred at RT for 1 h, concentrated, the residue was taken up in CH₂Cl₂, neutralized with NaHCO₃ and adsorbed onto Celite®. Reverse phase chromatography (C18; 0-10% acetonitrile/water) afforded the product as a beige solid (84 mg, 51%); LCMS: [M+H]⁺=417.46.

Step 3: 6-(2-amino-6-fluoro-5-(4-(1-(2-methoxyethyl)piperidin-4-yl)phenyl)pyridin-3-yl)-3,4-dihydroisoquinolin-1(2H)-one

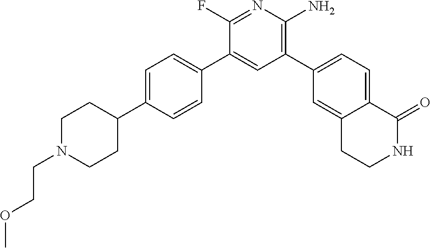 [SMILES: COCCN1CCC(c2ccc(-c3cc(-c4ccc5c(c4)CCNC5=O)c(N)nc3F)cc2)CC1]

A procedure analogous to that of Example 90, Step 2 was used by heating 6-(2-amino-6-fluoro-5-(4-(piperidin-4-yl)phenyl)pyridin-3-yl)-3,4-dihydroisoquinolin-1(2H)-one (28 mg, 0.067 mmol) and 2-bromoethyl methyl ether (0.013 mL, 0.134 mmol) in solvent at 80° C. for 6 h. The reaction mixture was partitioned between water (3 mL) and CH₂Cl₂ (3 mL). The layers were separated and the aqueous layer was further extracted with CH₂Cl₂ (x2). The combined organic extracts was dried and concentrated onto Celite®. Reverse phase chromatography (C18; 0-45% acetonitrile/water) afforded the title compound (20.5 mg, 61%) as a beige solid. ¹H NMR (500 MHz, DMSO-d₆) δ 7.93-8.00 (m, 1H), 7.91 (d, J=8.0 Hz, 1H), 7.61-7.67 (m, 1H), 7.41-7.53 (m, 4H), 7.25-7.35 (m, 2H), 6.22-6.36 (m, 2H), 3.45-3.50 (m, 2H), 3.40-3.44 (m, 2H), 3.37 (br s, 1H), 3.22-3.29 (m, 3H), 2.98-3.08 (m, 2H), 2.92-2.98 (m, 2H), 2.52-2.59 (m, 2H), 1.95-2.24 (m, 2H), 1.72-1.81 (m, 2H), 1.56-1.71 (m, 2H); LCMS: [M+H]⁺=475.60.

### Example 108: (R)-6-(2-amino-6-fluoro-5-(2-fluoro-4-(3-methylmorpholino)phenyl)pyridin-3-yl)-3,4-dihydroisoquinolin-1(2H)-one (I-108)

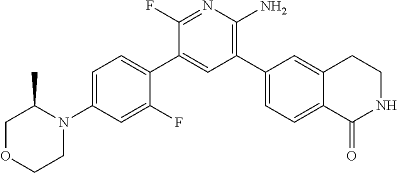 [SMILES: C[C@@H]1COCCN1c1ccc(-c2cc(-c3ccc4c(c3)CCNC4=O)c(N)nc2F)c(F)c1]

Step 1: (R)-4-(4-chloro-3-fluorophenyl)-3-methylmorpholine

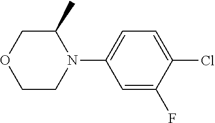

A 30 mL vial was charged with tris(dibenzylideneacetone)dipalladium (0) (0.16 g, 0.18 mmol), 4,5-bis(diphenylphosphino)-9,9-dimethylxanthene (0.31 g, 0.54 mmol) and Cs₂CO₃ (5.8 g, 18 mmol). The vial was sealed with a cap and septum and then the reaction vial was evacuated and backfilled with nitrogen. Toluene (14 mL), 4-bromo-1-chloro-2-fluorobenzene (0.83 mL, 7.2 mmol) and (R)-3-methylmorpholine (1.2 mL, 8.6 mmol) were added and the reaction vial was evacuated and backfilled with nitrogen an additional time. The reaction was heated at 95° C. for 18 h. The reaction mixture was cooled to RT, diluted with CH₂Cl₂ and filtered through Celite®. The filtrate was concentrated onto Celite® and purified by flash chromatography (1-15% EtOAc/hexanes) to afford the product (1.5 g, 88%). LCMS: [M+H]⁺=229.9.

Step 2: (R)-4-(3-fluoro-4-(4,4,5,5-tetramethyl-1,3,2-dioxaborolan-2-yl)phenyl)-3-methylmorpholine

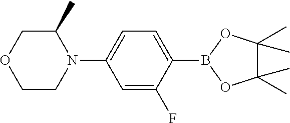 [SMILES: C[C@@H]1COCCN1c1ccc(B2OC(C)(C)C(C)(C)O2)c(F)c1]

A 30 mL vial was charged with (R)-4-(4-chloro-3-fluorophenyl)-3-methylmorpholine (1.2 g, 5.2 mmol), bis(pinacolato)diboron (1.9 g, 7.8 mmol), KOAc (1.0 g, 10 mmol) and XPhos Pd G2 (0.21 g, 0.26 mmol). The vial was evacuated and backfilled with nitrogen. 1,4-Dioxane (14 mL) was added and the vial was evacuated and backfilled an additional time. The reaction mixture was then heated to 110° C. in an aluminum block for 18 h. The reaction mixture was cooled to RT, diluted with CH₂Cl₂ and filtered through Celite®. The filtrate was concentrated onto Celite® and purified by flash chromatography (1-15% EtOAc/hexanes) to afford the product (1.5 g, 86%). LCMS: [M+H]⁺=322.1.

Step 3: (R)-6-(2-amino-6-fluoro-5-(2-fluoro-4-(3-methylmorpholino)phenyl)pyridin-3-yl)-3,4-dihydroisoquinolin-1(2H)-one

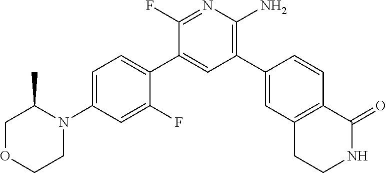 [SMILES: C[C@@H]1COCCN1c1ccc(-c2cc(-c3ccc4c(c3)CCNC4=O)c(N)nc2F)c(F)c1]

A 30 mL vial was charged (R)-4-(3-fluoro-4-(4,4,5,5-tetramethyl-1,3,2-dioxaborolan-2-yl)phenyl)-3-methylmorpholine (0.11 g, 0.34 mmol), 6-(2-amino-5-bromo-6-fluoropyridin-3-yl)-3,4-dihydroisoquinolin-1(2H)-one (0.10 g, 0.30 mmol) and [1,12-bis(diphenylphosphino)ferrocene]dichloropalladium(II) (0.022 g, 0.030 mmol). The vial was sealed with a cap and septum and the reaction vessel was evacuated and backfilled with nitrogen. 1,4-Dioxane (1 mL) and 2 M aqueous Na₂CO₃ (0.37 mL, 0.74 mmol) were added and the reaction vessel was evacuated and backfilled with nitrogen an additional time. The reaction mixture was heated at 90° C. for 18 h. The reaction mixture was cooled to RT, diluted with DCM and filtered through Celite®. The filtrate was concentrated onto Celite® and purified by flash chromatography (0.5-9.5% MeOH/DCM+0.5% NH₄OH). The product containing fractions were concentrated and further purified by reverse phase chromatography (Biotage SNAP C18; 5-85% MeCN/water+0.1% Formic Acid). Isolation of the title compound was achieved by a catch and release procedure using Biotage SCX2 silica gel to afford the title compound (15 mg, 11%) as an off-white powder. ¹H NMR (500 MHz, DMSO-d₆) δ 7.94 (br s, 1H), 7.90 (d, J=7.9 Hz, 1H), 7.4-7.5 (m, 3H), 7.29 (t, J=8.7 Hz, 1H), 6.7-6.8 (m, 2H), 6.27 (s, 2H), 3.9-4.0 (m, 2H), 3.7-3.7 (m, 2H), 3.54 (dt, J=11.5, 3.1 Hz, 1H), 3.41 (dt, J=6.5, 2.6 Hz, 2H), 3.02 (dt, J=12.0, 3.7 Hz, 1H), 2.95 (t, J=6.5 Hz, 2H), 1.05 (d, J=6.5 Hz, 3H); LCMS: [M+H]⁺=451.4.

### Example 109: 6-(2-amino-5-(4-(2,5-dihydro-1H-pyrrol-3-yl)phenyl)-6-fluoropyridin-3-yl)-3,4-dihydroisoquinolin-1(2H)-one 2,2,2-trifluoroacetate (1-109)

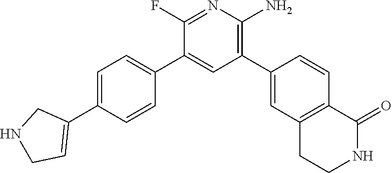 [SMILES: Nc1nc(F)c(-c2ccc(C3=CCNC3)cc2)cc1-c1ccc2c(c1)CCNC2=O]

### Step 1: tert-butyl3-(4-(6-amino-2-fluoro-5-(1-oxo-1,2,3,4-tetrahydroisoquinolin-6-yl)pyridin-3-yl)phenyl)-2,5-dihydro-1H-pyrrole-1-carboxylate

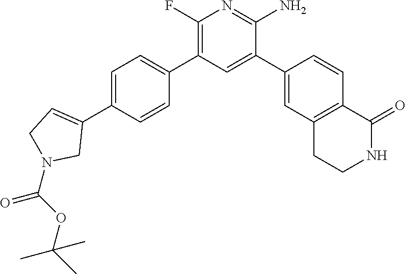 [SMILES: CC(C)(C)OC(=O)N1CC=C(c2ccc(-c3cc(-c4ccc5c(c4)CCNC5=O)c(N)nc3F)cc2)C1]

A procedure analogous to Example 43, Step 5 using tert-butyl 3-(4-bromophenyl)-2,5-dihydro-1H-pyrrole-1-carboxylate (350 mg, 1.08 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (538 mg, 1.40 mmol) in solvent at 90° C. for 1.25 h. The reaction mixture was partitioned between brine and EtOAc. The layers were separated and the aqueous layer was further extracted with EtOAc (x2). Silica gel chromatography (eluting with 0-100% EtOAc/hexanes) afforded the product (417 mg, 73%) as a beige solid. LCMS: [M+H]⁺=501.45

Step 2: 6-(2-amino-5-(4-(2,5-dihydro-1H-pyrrol-3-yl)phenyl)-6-fluoropyridin-3-yl)-3,4-dihydroisoquinolin-1(2H)-one (TFA salt)

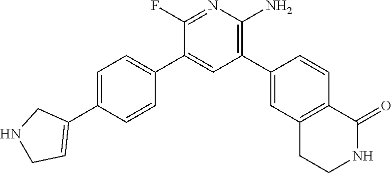 [SMILES: Nc1nc(F)c(-c2ccc(C3=CCNC3)cc2)cc1-c1ccc2c(c1)CCNC2=O]

A solution of tert-butyl 3-(4-(6-amino-2-fluoro-5-(1-oxo-1,2,3,4-tetrahydroisoquinolin-6-yl)pyridin-3-yl)phenyl)-2,5-dihydro-1H-pyrrole-1-carboxylate (417 mg, 0.791 mmol) at RT with CH₂Cl₂ (2 mL) was treated with TFA (0.5 mL) and the mixture was stirred for 30 min. The reaction mixture was concentrated onto Celite®. Reverse phase chromatography (C18; 0-20% acetonitrile/water) afforded the trifluoroacetic acid salt of the title compound as a beige solid (347 mg, 81%). ¹H NMR (500 MHz, DMSO-d₆) δ 9.49-9.31 (m, 2H), 8.02-7.95 (m, 1H), 7.95-7.89 (m, 1H), 7.73-7.67 (m, 1H), 7.66-7.61 (m, 2H), 7.60-7.55 (m, 2H), 7.52-7.47 (m, 1H), 7.46 (s, 1H), 6.51-6.46 (m, 1H), 6.44-6.35 (m, 2H), 4.46-4.35 (m, 2H), 4.24-4.14 (m, 2H), 3.46-3.40 (m, 2H), 3.02-2.94 (m, 2H); LCMS: [M +H]⁺=401.4.

### Example 110: 6-(2-amino-6-fluoro-5-(4-((1R,5S)-3-isopropyl-3-azabicyclo[3.1.0]hexan-1-yl)phenyl)pyridin-3-yl)-3,4-dihydroisoquinolin-1(2H)-one (I-110)

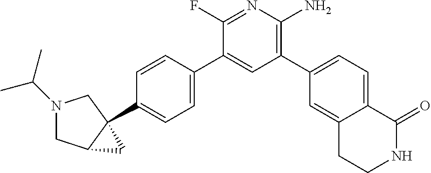 [SMILES: CC(C)N1C[C@H]2C[C@@]2(c2ccc(-c3cc(-c4ccc5c(c4)CCNC5=O)c(N)nc3F)cc2)C1]

Step 1: (1R,5S)-1-(4-bromophenyl)-3-isopropyl-3-azabicyclo[3.1.0]hexane

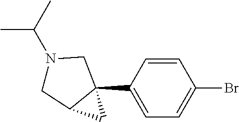 [SMILES: CC(C)N1C[C@H]2C[C@@]2(c2ccc(Br)cc2)C1]

A solution of (1R,5S)-1-(4-bromophenyl)-3-azabicyclo[3.1.0]hexane (200 mg, 0.840 mmol) in anh. CH₂Cl₂ (18 mL) was treated with acetone (0.12 mL, 1.7 mmol) at rt and glacial acetic acid (AcOH) (0.096 mL, 1.68 mmol). After stirring for 1 h at RT, NaBH(OAc)₃ (445 mg, 2.10 mmol) was added in one portion at RT and stirring was continued for 2 d. MeOH (2 mL) and satd aq NaHCO₃ (6 mL) were added. After separation of the layers, the aqueous phase was extracted with CH₂Cl₂. The combined organic layers were filtered through a plug of Na₂SO₄ and concentrated under reduced pressure to afford the product as a yellow solid (237.0 mg, quant). LCMS: [M+H]⁺=280.21/282.24.

Step 2: 6-(2-amino-6-fluoro-5-(4-((1R,5S)-3-isopropyl-3-azabicyclo[3.1.0]hexan-1-yl)phenyl)pyridin-3-yl)-3,4-dihydroisoquinolin-1(2H)-one

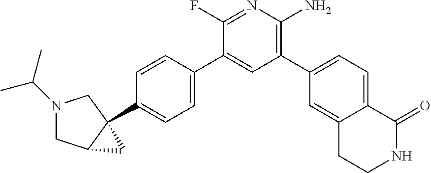 [SMILES: CC(C)N1C[C@H]2C[C@@]2(c2ccc(-c3cc(-c4ccc5c(c4)CCNC5=O)c(N)nc3F)cc2)C1]

A method similar to Example 1, General Method A using (1R,5S)-1-(4-bromophenyl)-3-isopropyl-3-azabicyclo[3.1.0]hexane (80 mg, 0.29 mmol), PdCl₂dppf (29 mg, 0.040 mmol), 6-(2-amino-6-fluoro-5-(4,4,5,5-tetramethyl-1,3,2-dioxaborolan-2-yl)pyridin-3-yl)-3,4-dihydroisoquinolin-1(2H)-one (115 mg, 0.300 mmol), Cs₂CO₃ (279 mg, 0.857 mmol), H₂O (2 mL) and DME (4 mL) afforded the title compound (37 mg, 27% yield based on purity of 94.4%). ¹H NMR (500 MHz, DMSO-d₆) δ 7.95 (br s, 1H), 7.91 (d, J=7.8 Hz, 1H), 7.62 (d, J=10.2 Hz, 1H), 7.42-7.50 (m, 4H), 7.19 (d, J=8.3 Hz, 2H), 6.29 (s, 2H), 3.41 (td, J=6.5, 2.7 Hz, 2H), 3.34-3.37 (m, 1H), 3.05 (d, J=8.6 Hz, 1H), 2.96 (t, J=6.5 Hz, 2H), 2.54-2.59 (m, 1H), 2.43-2.49 (m, 2H), 1.81 (dt, J=7.8, 3.9 Hz, 1H), 1.31 (t, J=3.9 Hz, 1H), 1.03 (dd, J=13.8, 6.3 Hz, 6H), 0.74 (dd, J=7.9, 3.6 Hz, 1H). LCMS: [M+H]⁺=457.42.

### Example 111: 6-(2-amino-6-fluoro-5-(4-((1S,5R)-3-isopropyl-3-azabicyclo[3.1.0]hexan-1-yl)phenyl)pyridin-3-yl)-3,4-dihydroisoquinolin-1(2H)-one 2,2,2-trifluoroacetate (I-111)

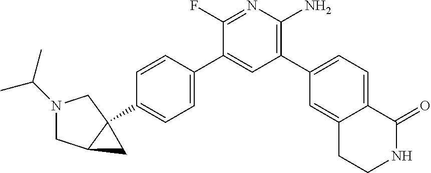 [SMILES: CC(C)N1C[C@@H]2C[C@]2(c2ccc(-c3cc(-c4ccc5c(c4)CCNC5=O)c(N)nc3F)cc2)C1]

Step 1: (1S,5R)-1-(4-bromophenyl)-3-isopropyl-3-azabicyclo[3.1.0]hexane

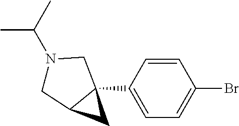 [SMILES: CC(C)N1C[C@@H]2C[C@]2(c2ccc(Br)cc2)C1]

A procedure analogous to Example 110, Step 1 using (1S,5R)-1-(4-bromophenyl)-3-azabicyclo[3.1.0]hexane (200 mg, 0.840 mmol), anh. CH₂Cl₂ (18 mL), acetone (0.12 mL, 1.68 mmol), glacial acetic acid (0.096 mL, 1.68 mmol) and NaBH(OAc)₃ (445 mg, 2.100 mmol) afforded the product as a red solid (282.0 mg, quant based on purity of 83%) that was used without further purification in the following step. LCMS: [M+H]⁺=280.21/282.24.

Step 2: 6-(2-Amino-6-fluoro-5-(4-((1S,5R)-3-isopropyl-3-azabicyclo[3.1.0]hexan-1-yl)phenyl)pyridin-3-yl)-3,4-dihydroisoquinolin-1(2H)-one·TFA

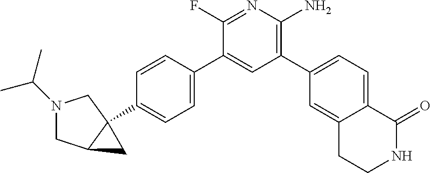 [SMILES: CC(C)N1C[C@@H]2C[C@]2(c2ccc(-c3cc(-c4ccc5c(c4)CCNC5=O)c(N)nc3F)cc2)C1]

A procedure similar to Example 1, General Method A using (1S,5R)-1-(4-bromophenyl)-3-isopropyl-3-azabicyclo[3.1.0]hexane (80 mg, 0.24 mmol, 83% purity), 6-(2-amino-6-fluoro-5-(4,4,5,5-tetramethyl-1,3,2-dioxaborolan-2-yl)pyridin-3-yl)-3,4-dihydroisoquinolin-1(2H)-one (95 mg, 0.249 mmol), PdCl₂dppf (24.3 mg, 0.033 mmol), Cs₂CO₃ (232 mg, 0.711 mmol), H₂O (2 mL) and DME (4 mL); by heating, degassed and sealed, in a microwave reactor at 90° C. for 1 h. The reaction mixture was concentrated purified by flash chromatography (25 g SiO₂, using CH₂Cl₂/MeOH/aq NH₄OH (89/10/1 v/v/v) in CH₂Cl₂) and then by preparative HPLC (30 g Biotage® SNAP KP-C₁₈-HS, MeOH in (H₂O +0.05% TFA)) to afford the title compound as a pale yellow solid (47 mg, 34% yield based on purity of 97.4%). ¹H NMR (500 MHz, CD₃OD) δ 7.93 (d, J=8.1 Hz, 1H), 7.52 (d, J=9.8 Hz, 1H), 7.43 (d, J=7.3 Hz, 2H), 7.36-7.40 (m, 1H), 7.35 (s, 1H), 7.27 (d, J=8.4 Hz, 2H), 4.00 (d, J=11.3 Hz, 1H), 3.72 (d, J=11.3 Hz, 1H), 3.52-3.61 (m, 2H), 3.34-3.48 (m, 3H), 2.95 (t, J=6.7 Hz, 2H), 2.09-2.19 (m, 1H), 1.32 (t, J=6.5 Hz, 6H), 1.15-1.23 (m, 2H); LCMS: [M+H]⁺=457.42.

### Example 112: (R)-6-(2-amino-6-fluoro-5-(4-(3-fluoropyrrolidin-1-yl)phenyl)pyridin-3-yl)-3,4-dihydroisoquinolin-1(2H)-one (I-112)

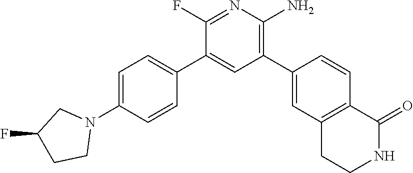 [SMILES: Nc1nc(F)c(-c2ccc(N3CC[C@@H](F)C3)cc2)cc1-c1ccc2c(c1)CCNC2=O]

Step 1: (R)-1-(4-chlorophenyl)-3-fluoropyrrolidine

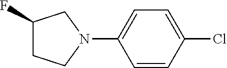 [SMILES: F[C@@H]1CCN(c2ccc(Cl)cc2)C1]

A procedure similar to Example 108, Step 1 using 1-chloro-4-iodobenzene (0.25 g, 1.0 mmol), tris(dibenzylideneacetone)dipalladium (0) (0.029 g, 0.031 mmol), 4,5-bis(diphenylphosphino)-9,9-dimethylxanthene (0.055 g, 0.094 mmol), Cs₂CO₃ (1.4 g, 4.2 mmol), toluene (5 mL) and (R)-(−)-3-fluoropyrrolidine hydrochloride (0.16 g, 1.3 mmol) afforded the product (150 mg, 73%). LCMS: [M+H]⁺=200.3.

Step 2: (R)-3-fluoro-1-(4-(4,4,5,5-tetramethyl-1,3,2-dioxaborolan-2-yl)phenyl)pyrrolidine

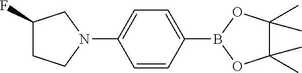 [SMILES: CC1(C)OB(c2ccc(N3CC[C@@H](F)C3)cc2)OC1(C)C]

A procedure analogous to Example 108, Step 3 using (R)-1-(4-chlorophenyl)-3-fluoropyrrolidine (0.15 g, 0.76 mmol), bis(pinacolato)diboron (0.29 g, 1.1 mmol), KOAc (0.15 g, 1.5 mmol) and XPhos Pd G2 (0.045 g, 0.057 mmol) afforded the product (140 mg, 64%). LCMS: [M+H]⁺=292.0.

Step 3: (R)-6-(2-amino-6-fluoro-5-(4-(3-fluoropyrrolidin-1-yl)phenyl)pyridin-3-yl)-3,4-dihydroisoquinolin-1(2H)-one

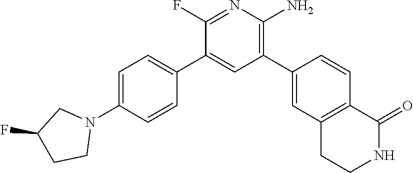 [SMILES: Nc1nc(F)c(-c2ccc(N3CC[C@@H](F)C3)cc2)cc1-c1ccc2c(c1)CCNC2=O]

A procedure analogous to Example 108, Step 3 using (R)-3-fluoro-1-(4-(4,4,5,5-tetramethyl-1,3,2-dioxaborolan-2-yl)phenyl)pyrrolidine (0.075 g, 0.26 mmol), 6-(2-amino-5-bromo-6-fluoropyridin-3-yl)-3,4-dihydroisoquinolin-1(2H)-one (0.075 g, 0.22 mmol), [1,12-bis(diphenylphosphino)ferrocene]dichloropalladium(II) (0.016 g, 0.022 mmol), 1,4-dioxane (1 mL) and 2 M aqueous Na₂CO₃ (0.28 mL, 0.56 mmol) afforded the title compound (1.0 mg, 1.1%). ¹H NMR (500 MHz, DMSO-d₆) δ 7.9-8.0 (m, 2H), 7.57 (d, J=10.1 Hz, 1H), 7.4-7.5 (m, 2H), 7.39 (d, J=7.8 Hz, 2H), 6.63 (d, J=8.7 Hz, 2H), 6.13 (s, 2H), 5.4-5.5 (m, 1H), 3.5-3.6 (m, 3H), 3.4-3.4 (m, 5H), 2.96 (t, J=6.5 Hz, 2H); LCMS: [M+H]⁺=421.4.

### Example 113: (S)-6-(2-amino-6-fluoro-5-(4-(3-fluoropyrrolidin-1-yl)phenyl)pyridin-3-yl)-3,4-dihydroisoquinolin-1(2H)-one (I-113)

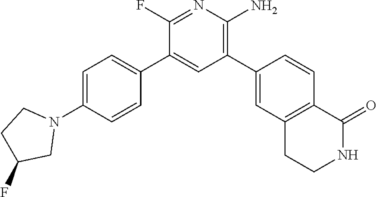 [SMILES: Nc1nc(F)c(-c2ccc(N3CC[C@H](F)C3)cc2)cc1-c1ccc2c(c1)CCNC2=O]

Step 1: (S)-1-(4-chlorophenyl)-3-fluoropyrrolidine

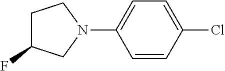 [SMILES: F[C@H]1CCN(c2ccc(Cl)cc2)C1]

A procedure analogous to Example 108, Step 1 using 1-chloro-4-iodobenzene (0.250 g, 1.05 mmol), tris(dibenzylideneacetone)dipalladium (0) (0.029 g, 0.031 mmol), 4,5-bis(diphenylphosphino)-9,9-dimethylxanthene (0.055 g, 0.094 mmol) and Cs₂CO₃ (1.366 g, 4.19 mmol) to afford the product (182 mg, 87%). LCMS: [M+H]⁺=200.18.

Step 2: (S)-3-fluoro-1-(4-(4,4,5,5-tetramethyl-1,3,2-dioxaborolan-2-yl)phenyl)pyrrolidine

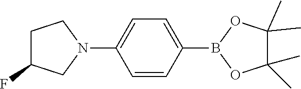 [SMILES: CC1(C)OB(c2ccc(N3CC[C@H](F)C3)cc2)OC1(C)C]

A procedure analogous to Example 108, Step 2 using (S)-1-(4-chlorophenyl)-3-fluoropyrrolidine (0.182 g, 0.912 mmol), bis(pinacolato)diboron (0.347 g, 1.37 mmol), KOAc (0.179 g, 1.823 mmol) and XPhos Pd G2 (0.054 g, 0.068 mmol afforded the product (168 mg, 63%). LCMS: [M+H]⁺=292.15.

Step 3: (S)-6-(2-amino-6-fluoro-5-(4-(3-fluoropyrrolidin-1-yl)phenyl)pyridin-3-yl)-3,4-dihydroisoquinolin-1(2H)-one

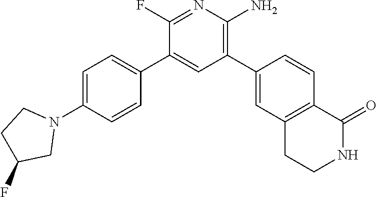 [SMILES: Nc1nc(F)c(-c2ccc(N3CC[C@H](F)C3)cc2)cc1-c1ccc2c(c1)CCNC2=O]

A procedure analogous to Example 43, Step 5 using (S)-3-fluoro-1-(4-(4,4,5,5-tetramethyl-1,3,2-dioxaborolan-2-yl)phenyl)pyrrolidine (0.075 g, 0.257 mmol), 6-(2-amino-5-bromo-6-fluoropyridin-3-yl)-3,4-dihydroisoquinolin-1(2H)-one (0.075 g, 0.223 mmol), [1,12-bis(diphenylphosphino)ferrocene]dichloropalladium(II) (0.016 g, 0.022 mmol), 1,4-dioxane (1 mL) and aqueous Na₂CO₃ (2M) (0.279 mL, 0.558 mmol) to afford the title compound (2 mg, 2.1%) as an off-white powder. ¹H NMR (500 MHz, DMSO-d₆) δ 7.8-7.9 (m, 2H), 7.49 (d, J=10.1 Hz, 1H), 7.4-7.4 (m, 2H), 7.32 (d, J=7.7 Hz, 2H), 6.55 (d, J=8.8 Hz, 2H), 6.05 (s, 2H), 5.3-5.5 (m, 1H), 3.4-3.6 (m, 2H), 3.3-3.4 (m, 4H), 2.88 (t, J=6.5 Hz, 2H); LCMS: [M+H]⁺=421.4.

### Example 114: (R)-6-(2-amino-6-fluoro-5-(4-(3-methoxypyrrolidin-1-yl)phenyl)pyridin-3-yl)-3,4-dihydroisoquinolin-1(2H)-one (I-114)

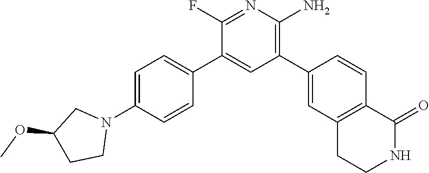 [SMILES: CO[C@@H]1CCN(c2ccc(-c3cc(-c4ccc5c(c4)CCNC5=O)c(N)nc3F)cc2)C1]

Step 1: (R)-3-methoxy-1-(4-(4,4,5,5-tetramethyl-1,3,2-dioxaborolan-2-yl)phenyl)pyrrolidine

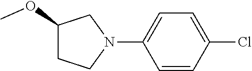

A procedure analogous to Example 108, Step 1 using 1-chloro-4-iodobenzene (0.250 g, 1.05 mmol), tris(dibenzylideneacetone)dipalladium (0) (0.029 g, 0.031 mmol), 4,5-bis(diphenylphosphino)-9,9-dimethylxanthene (0.055 g, 0.094 mmol), Cs₂CO₃ (1.37 g, 4.19 mmol) and (R)-3-methoxy-pyrrolidine hydrochloride (0.173 g, 1.258 mmol) afforded the product (202 mg, 91%). LCMS: [M+H]⁺=211.99.

Step 2: (R)-3-methoxy-1-(4-(4,4,5,5-tetramethyl-1,3,2-dioxaborolan-2-yl)phenyl)pyrrolidine

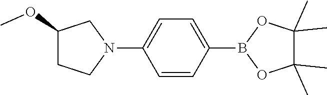

A procedure analogous to Example 108, Step 2 using (R)-1-(4-chlorophenyl)-3-methoxypyrrolidine (0.202 g, 0.954 mmol), bis(pinacolato)diboron (0.363 g, 1.431 mmol), KOAc (0.187 g, 1.91 mmol) and XPhos Pd G2 (0.056 g, 0.072 mmol) afforded the product (138 mg, 48%). LCMS: [M+H]⁺=303.95.

Step 3: (R)-6-(2-amino-6-fluoro-5-(4-(3-methoxypyrrolidin-1-yl)phenyl)pyridin-3-yl)-3,4-dihydroisoquinolin-1(2H)-one

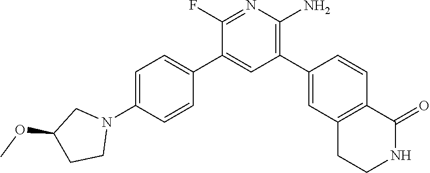 [SMILES: CO[C@@H]1CCN(c2ccc(-c3cc(-c4ccc5c(c4)CCNC5=O)c(N)nc3F)cc2)C1]

A procedure analogous to Example 108, Step 3 using (R)-3-methoxy-1-(4-(4,4,5,5-tetramethyl-1,3,2-dioxaborolan-2-yl)phenyl)pyrrolidine (0.078 g, 0.257 mmol), 6-(2-amino-5-bromo-6-fluoropyridin-3-yl)-3,4-dihydroisoquinolin-1(2H)-one (0.075 g, 0.223 mmol), [1,12-bis(diphenylphosphino)ferrocene]dichloropalladium(II) (0.016 g, 0.022 mmol) and aqueous Na₂CO₃ (0.279 ml of a 2M solution, 0.558 mmol) afforded the title compound (20 mg, 21%) as a off-white powder. ¹H NMR (500 MHz, DMSO-d₆) δ 7.9-8.0 (m, 2H), 7.56 (d, J=10.1 Hz, 1H), 7.4-7.5 (m, 2H), 7.37 (d, J=8.1 Hz,), 6.59 (d, J=8.7 Hz, 2H), 6.11 (s, 2H), 4.10 (br d, J=3.2 Hz, 1H), 3.4-3.5 (m, 4H), 3.2-3.3 (m, 6H), 2.96 (br t, J=6.5 Hz, 2H), 2.0-2.1 (m, 2H); LCMS: [M+H]⁺=433.3.

### Example 115: (S)-6-(2-amino-6-fluoro-5-(4-(3-methoxypyrrolidin-1-yl)phenyl)pyridin-3-yl)-3,4-dihydroisoquinolin-1(2H)-one (I-115)

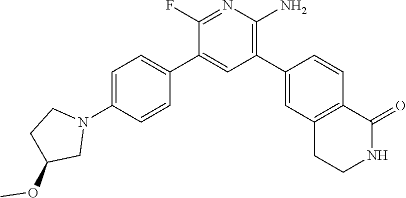 [SMILES: CO[C@H]1CCN(c2ccc(-c3cc(-c4ccc5c(c4)CCNC5=O)c(N)nc3F)cc2)C1]

Step 1: (S)-1-(4-chlorophenyl)-3-methoxypyrrolidine

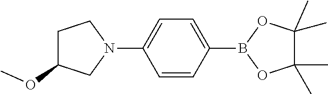

A procedure analogous to Example 108, Step 1 using 1-chloro-4-iodobenzene (0.250 g, 1.05 mmol), tris(dibenzylideneacetone)dipalladium (0) (0.029 g, 0.031 mmol), 4,5-bis(diphenylphosphino)-9,9-dimethylxanthene (0.055 g, 0.094 mmol) and CS₂CO₃ (1.37 g, 4.19 mmol) and Cs₂CO₃ (1.37 g, 4.19 mmol) afforded the product (246 mg, quantitative yield). LCMS: [M+H]⁺=211.91.

Step 2: (S)-3-Methoxy-1-(4-(4,4,5,5-tetramethyl-1,3,2-dioxaborolan-2-yl)phenyl)pyrrolidine

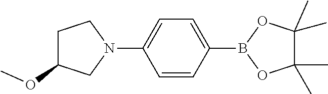

A procedure analogous to Example 108, Step 2 using (S)-1-(4-chlorophenyl)-3-methoxypyrrolidine (0.246 g, 1.16 mmol), bis(pinacolato)diboron (0.443 g, 1.74 mmol), KOAc (0.228 g, 2.32 mmol) and XPhos Pd G2 (0.069 g, 0.087 mmol) afforded the product (176 mg, 50%). LCMS: [M+H]⁺=304.03.

Step 3: (S)-6-(2-amino-6-fluoro-5-(4-(3-methoxypyrrolidin-1-yl)phenyl)pyridin-3-yl)-3,4-dihydroisoquinolin-1(2H)-one

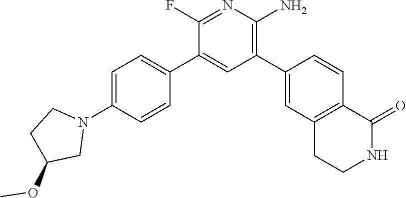 [SMILES: CO[C@H]1CCN(c2ccc(-c3cc(-c4ccc5c(c4)CCNC5=O)c(N)nc3F)cc2)C1]

A procedure analogous to Example 108, Step 3 using (S)-3-methoxy-1-(4-(4,4,5,5-tetramethyl-1,3,2-dioxaborolan-2-yl)phenyl)pyrrolidine (0.078 g, 0.257 mmol), 6-(2-amino-5-bromo-6-fluoropyridin-3-yl)-3,4-dihydroisoquinolin-1(2H)-one (0.075 g, 0.223 mmol), [1,12-bis(diphenylphosphino)ferrocene]dichloropalladium(II) (0.016 g, 0.022 mmol) and aqueous Na₂CO₃ (0.279 mL of a 2.0 M solution, 0.558 mmol) afforded the title compound (15 mg, 16%) as an off-white powder. ¹H NMR (500 MHz, DMSO-d₆) δ 7.9-8.0 (m, 2H), 7.56 (d, J=10.1 Hz, 1H,), 7.4-7.5 (m, 2H), 7.37 (d, J=7.7 Hz, 2H), 6.59 (d, J=8.8 Hz, 2H), 6.11 (s, 2H), 4.1-4.1 (m, 1H), 3.4-3.5 (m, 4H), 3.2-3.3 (m, 6H), 2.96 (t, J=6.5 Hz, 2H), 2.0-2.1 (m, 2H); LCMS: [M+H]⁺=433.3.

### Example 116: (R)-6-(2-amino-6-fluoro-5-(4-(3-(methylsulfonyl)pyrrolidin-1-yl)phenyl)pyridin-3-yl)-3,4-dihydroisoquinolin-1(2H)-one (I-116)

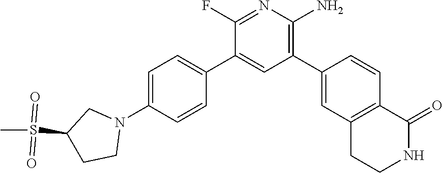 [SMILES: CS(=O)(=O)[C@@H]1CCN(c2ccc(-c3cc(-c4ccc5c(c4)CCNC5=O)c(N)nc3F)cc2)C1]

Step 1: (R)-1-(4-chlorophenyl)-3-(methylsulfonyl)pyrrolidine

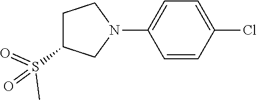 [SMILES: CS(=O)(=O)[C@@H]1CCN(c2ccc(Cl)cc2)C1]

A procedure analogous to Example 15, Step 1 using 1-chloro-4-iodobenzene (0.250 g, 1.05 mmol), tris(dibenzylideneacetone)dipalladium (0) (0.029 g, 0.031 mmol), 4,5-bis(diphenylphosphino)-9,9-dimethylxanthene (0.055 g, 0.094 mmol) and Cs₂CO₃ (1.03 g, 3.15 mmol) and (R)-3-(methylsulfonyl)pyrrolidine (0.188 g, 1.258 mmol) afforded the product (183 mg, 67%). LCMS: [M+H]⁺=259.96.

Step 2: (R)-3-(methylsulfonyl)-1-(4-(4,4,5,5-tetramethyl-1,3,2-dioxaborolan-2-yl)phenyl)pyrrolidine

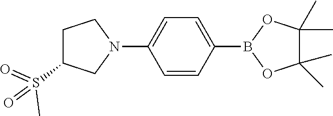 [SMILES: CC1(C)OB(c2ccc(N3CC[C@@H](S(C)(=O)=O)C3)cc2)OC1(C)C]

A procedure analogous to Example 15, Step 2 using (R)-1-(4-chlorophenyl)-3-(methylsulfonyl)pyrrolidine (0.183 g, 0.705 mmol), bis(pinacolato)diboron (0.268 g, 1.057 mmol), KOAc (0.138 g, 1.409 mmol) and XPhos Pd G2 (0.042 g, 0.053 mmol) afforded the product (166 mg, 67%). LCMS: [M+H]⁺=352.24.

Step 3: (R)-6-(2-amino-6-fluoro-5-(4-(3-(methylsulfonyl)pyrrolidin-1-yl)phenyl)pyridin-3-yl)-3,4-dihydroisoquinolin-1(2H)-one

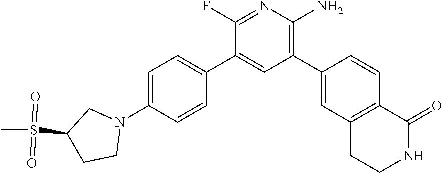 [SMILES: CS(=O)(=O)[C@@H]1CCN(c2ccc(-c3cc(-c4ccc5c(c4)CCNC5=O)c(N)nc3F)cc2)C1]

A procedure analogous to Example 108, Step 3 using (R)-3-(methylsulfonyl)-1-(4-(4,4,5,5-tetramethyl-1,3,2-dioxaborolan-2-yl)phenyl)pyrrolidine (0.090 g, 0.257 mmol), 6-(2-amino-5-bromo-6-fluoropyridin-3-yl)-3,4-dihydroisoquinolin-1(2H)-one (0.075 g, 0.223 mmol) and [1,12-bis(diphenylphosphino)ferrocene]dichloropalladium(II) (0.016 g, 0.022 mmol) and aqueous Na₂CO₃ (0.279 mL of a 2M solution, 0.558 mmol) afforded the title compound (1 mg, 1.0%). ¹H NMR (500 MHz, DMSO-d₆) δ 7.9-8.0 (m, 2H), 7.58 (br d, J=10.0 Hz, 1H), 7.4-7.5 (m, 2H), 7.41 (br d, J=8.4 Hz, 2H), 6.68 (br d, J=8.2 Hz, 2H), 6.14 (br s, 2H), 4.1-4.1 (m, 2H), 3.63 (br d, J=7.1 Hz, 4H), 3.07 (s, 3H), 2.9-3.0 (m, 4H); LCMS: [M+H]⁺=481.4.

### Example 117: (S)-6-(2-amino-6-fluoro-5-(4-(3-(methylsulfonyl)pyrrolidin-1-yl)phenyl)pyridin-3-yl)-3,4-dihydroisoquinolin-1(2H)-one (I-117)

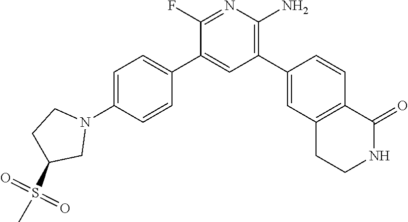 [SMILES: CS(=O)(=O)[C@H]1CCN(c2ccc(-c3cc(-c4ccc5c(c4)CCNC5=O)c(N)nc3F)cc2)C1]

Step 1: (S)-1-(4-chlorophenyl)-3-(methylsulfonyl)pyrrolidine

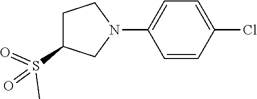 [SMILES: CS(=O)(=O)[C@H]1CCN(c2ccc(Cl)cc2)C1]

A procedure analogous to Example 15, Step 1 using 1-chloro-4-iodobenzene (0.250 g, 1.05 mmol), tris(dibenzylideneacetone)dipalladium (0) (0.029 g, 0.031 mmol), 4,5-bis(diphenylphosphino)-9,9-dimethylxanthene (0.055 g, 0.094 mmol), Cs₂CO₃ (1.03 g, 3.15 mmol) and (S)-3-(methylsulfonyl)pyrrolidine (0.188 g, 1.6 mmol) afforded the product (191 mg, 70%). LCMS: [M+H]⁺=352.17.

Step 2: (S)-3-(methylsulfonyl)-1-(4-(4,4,5,5-tetramethyl-1,3,2-dioxaborolan-2-yl)phenyl)pyrrolidine

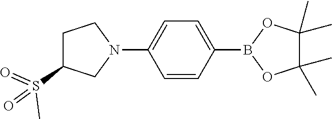 [SMILES: CC1(C)OB(c2ccc(N3CC[C@H](S(C)(=O)=O)C3)cc2)OC1(C)C]

A procedure analogous to Example 15, Step 2 using (S)-1-(4-chlorophenyl)-3-(methylsulfonyl)pyrrolidine (0.191 g, 0.735 mmol), bis(pinacolato)diboron (0.280 g, 1.10 mmol), KOAc (0.144 g, 1.47 mmol) and XPhos Pd G2 (0.043 g, 0.055 mmol) afforded the product (200 mg, 77%). LCMS: [M+H]⁺=352.17.

Step 3: (S)-6-(2-amino-6-fluoro-5-(4-(3-(methylsulfonyl)pyrrolidin-1-yl)phenyl)pyridin-3-yl)-3,4-dihydroisoquinolin-1(2H)-one

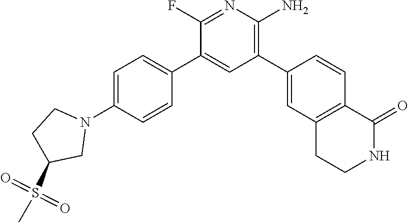 [SMILES: CS(=O)(=O)[C@H]1CCN(c2ccc(-c3cc(-c4ccc5c(c4)CCNC5=O)c(N)nc3F)cc2)C1]

A procedure analogous to Example 108, Step 3 using (S)-3-(methylsulfonyl)-1-(4-(4,4,5,5-tetramethyl-1,3,2-dioxaborolan-2-yl)phenyl)pyrrolidine (0.090 g, 0.257 mmol), 6-(2-amino-5-bromo-6-fluoropyridin-3-yl)-3,4-dihydroisoquinolin-1(2H)-one (0.075 g, 0.223 mmol) and [1,12-bis(diphenylphosphino)ferrocene]dichloropalladium(II) (0.016 g, 0.022 mmol) and aqueous Na₂CO₃ (0.279 ml of a 2M solution, 0.558 mmol) afforded the title compound (1 mg, 1.0%). ¹H NMR (500 MHz, DMSO-d₆) δ 7.9-8.0 (m, 2H), 7.58 (br d, J=10.1 Hz, 1H), 7.4-7.5 (m, 2H), 7.41 (br d, J=8.3 Hz, 2H), 6.68 (br d, J=8.1 Hz, 2H), 6.14 (br s, 2H), 4.1-4.1 (m, 2H), 3.63 (br d, J=7.0 Hz, 3H), 3.07 (s, 3H), 2.96 (br t, J=6.1 Hz, 4H,); LCMS: [M+H]⁺=481.4.

### Example 118: 6-(2-amino-6-fluoro-5-(4-(4-methyl-5,6-dihydro-1,2,4-triazin-1(4H)-yl)phenyl)pyridin-3-yl)-3,4-dihydroisoquinolin-1(2H)-one (I-118)

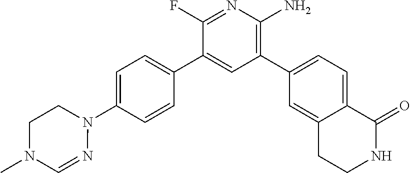 [SMILES: CN1C=NN(c2ccc(-c3cc(-c4ccc5c(c4)CCNC5=O)c(N)nc3F)cc2)CC1]

Step 1: 2-(1-(4-nitrophenyl)hydrazineyl)ethan-1-ol:

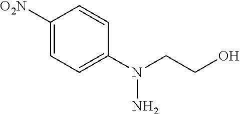 [SMILES: NN(CCO)c1ccc([N+](=O)[O-])cc1]

To a stirred solution of 1-fluoro-4-nitrobenzene (37.5 mL, 354.6 mmol) in CH₃CN (500 mL) was added 2-hydrazineylethan-1-ol (27.1 mL, 459.9 mmol) followed by the addition of K₂CO₃ (54.2 g, 392.2 mmol) under argon atmosphere and stirred at 85° C. for 16 h before cooled to RT. The reaction mixture was filtered through Celite® and washed with EtOAc (500 mL). The combined filtrates were concentrated under reduce pressure to afford crude product (38 g, 54%) as an orange gummy liquid which was used without further purification for the next step. LCMS: [M+H]⁺=198.39.

Step 2: 2-(1-(4-nitrophenyl)hydrazineyl)ethan-1-ol hydrobromide:

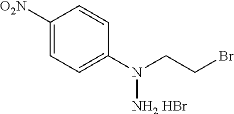

To a stirred solution of crude 2-(1-(4-nitrophenyl)hydrazineyl)ethan-1-ol (45 g, 228.4 mmol) in EtOAc (450 mL) was added 47% aqueous HBr solution (45 mL) at 0° C. and the mixture was stirred at 0° C. for 2 h. The precipitate was collected by filtration and washed with EtOAc (500 mL) to afford the product (41 g, 65%) as a yellow solid. LCMS: [M+H]⁺=198.14.

Step 3: 1-(2-bromoethyl)-1-(4-nitrophenyl) hydrazine:

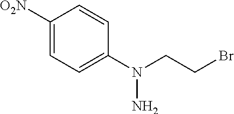 [SMILES: NN(CCBr)c1ccc([N+](=O)[O-])cc1]

To a stirred solution of 2-(1-(4-nitrophenyl)hydrazineyl)ethan-1-ol hydrobromide (42 g, 155.5 mmol) in dry DME (420 mL) was added PBr₃ (19 mL) dropwise at 0° C. under an argon atmosphere and then the reaction mixture was stirred at RT for 30 min and heated to 60° C. for 16 h before cooling to RT. The solvent was evaporated to dryness under reduced pressure to afford a gummy liquid, which was diluted with water (100 mL) and basified with NaHCO₃ solution at 0° C. The resulting precipitate was filtered off and washed with water, followed by n-pentane, and dried under vacuum to afford the product (28 g, 71%) as a yellow solid. LCMS: [M+2H]⁺=260.10.

Step 4: 1-(4-nitrophenyl)-1,4,5,6-tetrahydro-1,2,4-triazine:

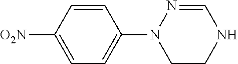 [SMILES: O=[N+]([O-])c1ccc(N2CCNC=N2)cc1]

To a stirred solution of crude 1-(2-bromoethyl)-1-(4-nitrophenyl) hydrazine (28 g, 108.1 mmol) in anhydrous DME (300 mL), was added ethyl formimidate hydrochloride (11.8 g, 108.1 mmol) under an argon atmosphere, and the mixture was stirred at RT for 20 min. Anhydrous NaOAc (18.6 g, 227 mmol) was added, and the reaction mixture was heated at reflux at 90° C. for 16 h and allowed to cool to RT. The solvent was evaporated to dryness to obtain a gummy liquid. The mixture was acidified with 1N HCl to pH=2 and washed with 60% EtOAc: pet ether (2×500 mL). The aqueous layer was adjusted to pH=8.5 by the addition of aqueous Na₂CO₃ at 0° C. to give a solid precipitate, which was collected by filtration and washed with n-pentane to afford the product (10.5 g, 46%) as an orange solid. LCMS: [M+H]⁺=207.21.

Step 5: 4-methyl-1-(4-nitrophenyl)-1,4,5,6-tetrahydro-1,2,4-triazine:

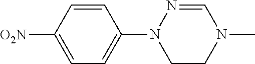 [SMILES: CN1C=NN(c2ccc([N+](=O)[O-])cc2)CC1]

To a stirred solution of 1-(4-nitrophenyl)-1,4,5,6-tetrahydro-1,2,4-triazine (10.9 g, 53 mmol) in DMF (120 mL) was added NaH 60% suspension in mineral oil (3.18 g, 79.5 mmol) under argon atmosphere at 0° C. stirred for 30 min at same temperature and then added methyl iodide (6.9 mL, 111.3 mmol). The mixture was stirred at RT for 4 h, then poured into ice. The resulting precipitate was collected by filtration and washed with cold water followed by n-pentane to afford the product (9.4 g, 81%) as an orange solid. LCMS: [M+H]⁺=221.14.

Step 6: 4-(4-methyl-5,6-dihydro-1,2,4-triazin-1(4H)-yl)aniline:

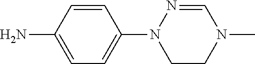

To a stirred solution of 4-methyl-1-(4-nitrophenyl)-1,4,5,6-tetrahydro-1,2,4-triazine (2.4 g, 10.9 mmol) in ethanol: methanol (125 mL: 250 mL) was added PtO₂ (990 mg, 4.36 mmol) and the mixture was stirred under a H₂ balloon pressure for 3 h at RT. The reaction mixture was filtered through Celite® bed and washed with methanol (200 mL). The combined filtrates were evaporated under reduced pressure to give the crude residue, which was triturated with diethyl ether to afford the desired aniline (1.36 g, 68%) as a brown solid. LCMS: [M+H]⁺=191.10.

Step 7: 1-(4-bromophenyl)-4-methyl-1,4,5,6-tetrahydro-1,2,4-triazine:

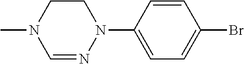

To a stirred solution of 4-(4-methyl-5,6-dihydro-1,2,4-triazin-1(4H)-yl)aniline (500 mg, 2.63 mmol) in CH₃CN (10 mL) was added Cu(I)Br (752 mg, 5.26 mmol) followed by tert-butyl nitrite (1.56 mL, 13.15 mmol) at 0° C. The reaction mixture was stirred at 0° C. for 2 h, then quenched with water and extracted with EtOAc (2×100 mL). The combined organic layer was dried over Na₂SO₄ and concentrated under reduced pressure to give crude residue which was purified by Prep HPLC to afford the product (30 mg, 4.5%) as a yellow solid. LCMS: [M+H]⁺=254.00.

Step 8: 6-(2-amino-6-fluoro-5-(4-(4-methyl-5,6-dihydro-1,2,4-triazin-1(4H)-yl)phenyl)pyridin-3-yl)-3,4-dihydroisoquinolin-1(2H)-one

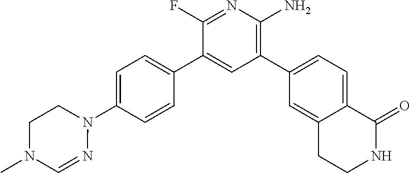 [SMILES: CN1C=NN(c2ccc(-c3cc(-c4ccc5c(c4)CCNC5=O)c(N)nc3F)cc2)CC1]

A procedure similar to Example 1, General Method A using 6-(2-amino-6-fluoro-5-(4,4,5,5-tetramethyl-1,3,2-dioxaborolan-2-yl)pyridin-3-yl)-3,4-dihydroisoquinolin-1(2H)-one (106 mg, 0.276 mmol), 1-(4-bromophenyl)-4-methyl-1,4,5,6-tetrahydro-1,2,4-triazine (54 mg, 0.212 mmol), Pd(amphos)Cl₂ (15.0 mg, 0.021 mmol), K₃PO₄ (135 mg, 0.637 mmol) in H₂O (1 mL) and 1,4-dioxane (8 mL) afforded after chromatographic purification the title compound as a pale yellow solid (8 mg, 8% yield based on purity of 96%). ¹H NMR (500 MHz, DMSO-d₆) δ 7.87 (br s, 1H), 7.83 (d, J=8.0 Hz, 1H), 7.51 (d, J=10.3 Hz, 1H), 7.35-7.47 (m, 2H), 7.31 (br d, J=7.8 Hz, 2H), 7.08 (d, J=8.9 Hz, 2H), 6.71 (s, 1H), 6.08 (s, 2H), 3.47-3.58 (m, 2H), 3.34 (td, J=6.5, 2.7 Hz, 2H), 3.29 (br t, J=5.0 Hz, 2H), 2.83-2.94 (m, 2H), 2.79 (s, 3H). LCMS: [M+H]⁺=431.39.

### Example 119: 6-(2-amino-6-fluoro-5-(4-(1-(2-hydroxyethyl)piperidin-4-yl)phenyl)pyridin-3-yl)-3,4-dihydroisoquinolin-1(2H)-one (I-119)

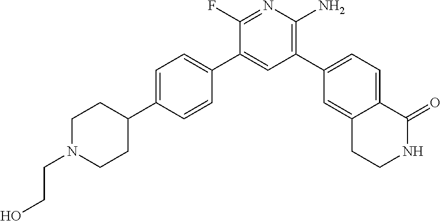 [SMILES: Nc1nc(F)c(-c2ccc(C3CCN(CCO)CC3)cc2)cc1-c1ccc2c(c1)CCNC2=O]

A procedure analogous to Example 90, Step 2 heating 6-(2-amino-6-fluoro-5-(4-(piperidin-4-yl)phenyl)pyridin-3-yl)-3,4-dihydroisoquinolin-1(2H)-one (30 mg, 0.072 mmol) and 2-bromoethanol, 95% (27.0 mg, 0.216 mmol) at 80° C. for 1 h afforded, after workup and purification, the title compound as an off white solid (17.5 mg, 50%). ¹H NMR (500 MHz, DMSO-d₆) δ 8.00-7.93 (m, 1H), 7.93-7.89 (m, 1H), 7.67-7.61 (m, 1H), 7.51-7.45 (m, 4H), 7.33-7.28 (m, 2H), 6.32-6.21 (m, 2H), 4.49-4.31 (m, 1H), 3.56-3.50 (m, 2H), 3.44-3.40 (m, 2H), 3.04-2.93 (m, 4H), 2.50-2.47 (m, 1H), 2.46-2.35 (m, 2H), 2.15-2.01 (m, 2H), 1.78-1.73 (m, 2H), 1.72-1.62 (m, 2H). LCMS: [M+H]⁺=461.46.

### Example 120: 6-(2-amino-5-(4-(1-ethyl-2,5-dihydro-1H-pyrrol-3-yl)phenyl)-6-fluoropyridin-3-yl)-3,4-dihydroisoquinolin-1(2H)-one (I-120)

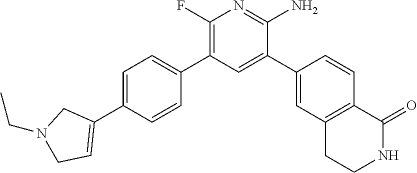 [SMILES: CCN1CC=C(c2ccc(-c3cc(-c4ccc5c(c4)CCNC5=O)c(N)nc3F)cc2)C1]

A procedure analogous to Example 90, Step 2 was followed by heating 6-(2-amino-5-(4-(2,5-dihydro-1 H-pyrrol-3-yl)phenyl)-6-fluoropyridin-3-yl)-3,4-dihydroisoquinolin-1(2H)-one TFA salt (45 mg, 0.087 mmol) and bromoethane (0.019 mL, 0.262 mmol) for 4.5 h at RT. Standard workup and purification afforded the title compound as an off-white powder (20.5 mg, 52%). ¹H NMR (500 MHz, DMSO-d₆) δ 7.99-7.94 (m, 1H), 7.93-7.90 (m, 1H), 7.70-7.65 (m, 1H), 7.57-7.53 (m, 2H), 7.51-7.44 (m, 4H), 6.50-6.28 (m, 3H), 3.85-3.74 (m, 2H), 3.64-3.55 (m, 2H), 3.44-3.40 (m, 2H), 3.01-2.94 (m, 2H), 2.67 (q, J=7.2 Hz, 2H), 1.16-1.06 (m, 3H). LCMS: [M+H]⁺=429.43.

### Example 121: 6-(2-amino-6-fluoro-5-(4-(1-isopropyl-2,5-dihydro-1H-pyrrol-3-yl)phenyl)pyridin-3-yl)-3,4-dihydroisoquinolin-1(2H)-one (I-121)

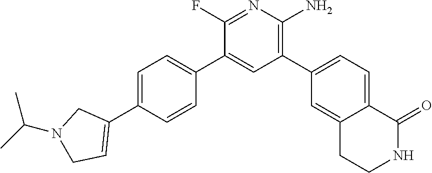 [SMILES: CC(C)N1CC=C(c2ccc(-c3cc(-c4ccc5c(c4)CCNC5=O)c(N)nc3F)cc2)C1]

A procedure analogous to Example 90, Step 2 was used by heating 6-(2-amino-5-(4-(2,5-dihydro-1 H-pyrrol-3-yl)phenyl)-6-fluoropyridin-3-yl)-3,4-dihydroisoquinolin-1(2H)-one TFA (45 mg, 0.087 mmol) and 2-iodopropane (0.035 mL, 0.350 mmol) overnight at RT. The reaction mixture was partitioned between water (3 mL) and CH₂Cl₂ (3 mL). The layers were separated and the aqueous layer was further extracted with CH₂Cl₂ (x3). The combined organic layers were dried over Na₂SO₄ filtered and concentrated onto Celite®. Reverse phase chromatography (C18; 0-50% acetonitrile/water) followed by silica gel chromatography (eluting with CH₂Cl₂ containing 0-4% MeOH and 0-0.4% NH₄OH) afforded the title compound as an off white solid (17.5 mg, 43%). ¹H NMR (500 MHz, DMSO-d₆) δ 8.00-7.93 (m, 1H), 7.93-7.88 (m, 1H), 7.73-7.64 (m, 1H), 7.58-7.52 (m, 2H), 7.52-7.44 (m, 4H), 6.46-6.27 (m, 3H), 3.86-3.74 (m, 2H), 3.64-3.55 (m, 2H), 3.45-3.39 (m, 2H), 3.01-2.94 (m, 2H), 2.75-2.64 (m, 1H), 1.17-1.00 (m, 6H); LCMS: [M+H]⁺=443.47.

### Example 122: 6-(2-amino-6-fluoro-5-(4-(1-(2-methoxyethyl)piperidin-3-yl)phenyl)pyridin-3-yl)-3,4-dihydroisoquinolin-1(2H)-one (I-122)

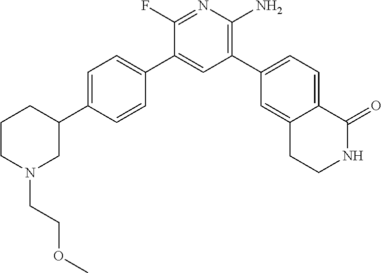 [SMILES: COCCN1CCCC(c2ccc(-c3cc(-c4ccc5c(c4)CCNC5=O)c(N)nc3F)cc2)C1]

A procedure analogous to that of Example 90, Step 2 by treating 6-(2-amino-6-fluoro-5-(4-(piperidin-3-yl)phenyl)pyridin-3-yl)-3,4-dihydroisoquinolin-1(2H)-one (45 mg, 0.108 mmol, preparation described in Example 93, Step 1) and 2-bromoethyl methyl ether (0.030 mL, 0.324 mmol) overnight at RT. The reaction was worked up and the crude product was purified by reverse phase chromatography (C18; 0-10% acetonitrile/water) to afford the title compound as a beige solid (31 mg, 57%). ¹H NMR (500 MHz, DMSO-d₆) δ 7.98-7.93 (m, 1H), 7.93-7.89 (m, 1H), 7.66-7.61 (m, 1H), 7.52-7.43 (m, 4H), 7.34-7.29 (m, 2H), 6.37-6.23 (m, 2H), 3.48-3.43 (m, 2H), 3.43-3.40 (m, 2H), 3.26-3.20 (m, 3H), 3.00-2.86 (m, 4H), 2.81-2.71 (m, 1H), 2.66-2.52 (m, 2H), 2.16-1.93 (m, 2H), 1.87-1.79 (m, 1H), 1.75-1.67 (m, 1H), 1.65-1.55 (m, 1H), 1.50-1.39 (m, 1H); LCMS: [M+H]⁺=475.50.

### Example 123: 6-(2-amino-6-fluoro-5-(4-(1-(2-hydroxyethyl)-2,5-dihydro-1H-pyrrol-3-yl)phenyl)pyridin-3-yl)-3,4-dihydroisoquinolin-1(2H)-one (I-123)

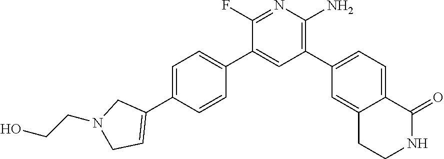 [SMILES: Nc1nc(F)c(-c2ccc(C3=CCN(CCO)C3)cc2)cc1-c1ccc2c(c1)CCNC2=O]

A procedure analogous to that of Example 90, Step 2 using 6-(2-amino-5-(4-(2,5-dihydro-1H-pyrrol-3-yl)phenyl)-6-fluoropyridin-3-yl)-3,4-dihydroisoquinolin-1(2H)-one-TFA (45 mg, 0.087 mmol) and 2-bromoethanol, 95% (0.019 mL, 0.262 mmol) and heating overnight afforded, after workup and purification, the title compound as an off-white solid (19 mg, 46%). ¹H NMR (500 MHz, DMSO-d₆) δ 8.00-7.94 (m, 1H), 7.93-7.87 (m, 1H), 7.70-7.65 (m, 1H), 7.58-7.52 (m, 2H), 7.51-7.41 (m, 4H), 6.50-6.28 (m, 3H), 4.56-4.43 (m, 1H), 3.86-3.77 (m, 2H), 3.67-3.61 (m, 2H), 3.57-3.50 (m, 2H), 3.46-3.39 (m, 2H), 2.96 (br t, J=6.2 Hz, 2H), 2.74 (br t, J=6.1 Hz, 2H); LCMS: [M+H]⁺=445.41

### Example 124: 6-(2-amino-6-fluoro-5-(4-(1-(2-methoxyethyl)-2,5-dihydro-1H-pyrrol-3-yl)phenyl)pyridin-3-yl)-3,4-dihydroisoquinolin-1(2H)-one (I-124)

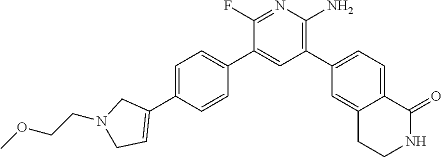 [SMILES: COCCN1CC=C(c2ccc(-c3cc(-c4ccc5c(c4)CCNC5=O)c(N)nc3F)cc2)C1]

A procedure analogous to that of Example 90, Step 2 stirring 6-(2-amino-5-(4-(2,5-dihydro-1H-pyrrol-3-yl)phenyl)-6-fluoropyridin-3-yl)-3,4-dihydroisoquinolin-1(2H)-one-TFA (45 mg, 0.087 mmol) and 2-bromoethyl methyl ether (0.025 mL, 0.262 mmol) for 16 h followed by workup and purification afforded the title compound as an off white solid (25.5 mg, 60%). ¹H NMR (500 MHz, DMSO-d₆) δ 7.99-7.94 (m, 1H), 7.93-7.87 (m, 1H), 7.70-7.65 (m, 1H), 7.58-7.53 (m, 2H), 7.51-7.43 (m, 4H), 6.40-6.28 (m, 3H), 5.49-5.48 (m, 1H), 3.82 (br s, 2H), 3.63 (br d, J=1.8 Hz, 2H), 3.50-3.45 (m, 2H), 3.41 (dt, J=6.4, 2.4 Hz, 2H), 3.30-3.26 (m, 3H), 2.96 (br t, J=6.4 Hz, 2H), 2.87-2.78 (m, 2H); LCMS: [M+H]+=459.45.

### Example 125: 6-(2-amino-6-fluoro-5-(4-((1R,5S)-3-(2-hydroxy-2-methylpropyl)-3-azabicyclo[3.1.0]hexan-1-yl)phenyl)pyridin-3-yl)-3,4-dihydroisoquinolin-1(2H)-one (I-125)

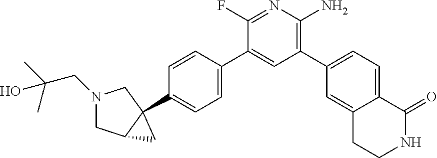 [SMILES: CC(C)(O)CN1C[C@H]2C[C@@]2(c2ccc(-c3cc(-c4ccc5c(c4)CCNC5=O)c(N)nc3F)cc2)C1]

Step 1: 1-((1R,5S)-1-(4-bromophenyl)-3-azabicyclo[3.1.0]hexan-3-yl)-2-methylpropan-2-ol

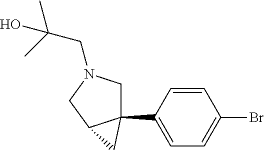 [SMILES: CC(C)(O)CN1C[C@H]2C[C@@]2(c2ccc(Br)cc2)C1]

To a MeOH (5 mL) solution of (1R,5S)-1-(4-bromophenyl)-3-azabicyclo[3.1.0]hexane (100 mg, 0.420 mmol) was added 1,2-epoxy-2-methylpropane (0.056 mL, 0.63 mmol) at rt. The reaction mixture was stirred at RT for 17 h then concentrated in vacuo to afford the product (130 mg, 85%) was used crude in the following step. LCMS: [M+H]⁺=310.24/312.27.

Step 2: 6-(2-amino-6-fluoro-5-(4-((1R,5S)-3-(2-hydroxy-2-methylpropyl)-3-azabicyclo[3.1.0]hexan-1-yl)phenyl)pyridin-3-yl)-3,4-dihydroisoquinolin-1(2H)-one

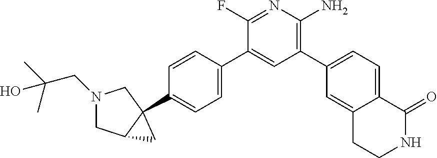 [SMILES: CC(C)(O)CN1C[C@H]2C[C@@]2(c2ccc(-c3cc(-c4ccc5c(c4)CCNC5=O)c(N)nc3F)cc2)C1]

A procedure similar to Example 1, General Method A using 1-((1R,5S)-1-(4-bromophenyl)-3-azabicyclo[3.1.0]hexan-3-yl)-2-methylpropan-2-ol (65 mg, 0.18 mmol, 85% purity), 6-(2-amino-6-fluoro-5-(4,4,5,5-tetramethyl-1,3,2-dioxaborolan-2-yl)pyridin-3-yl)-3,4-dihydroisoquinolin-1(2H)-one (82 mg, 0.21 mmol, PdCl₂dppf (13.0 mg, 0.018 mmol), Cs₂CO₃ (174 mg, 0.534 mmol), DME (4 mL) and H₂O (2 mL) afforded the title compound as a beige solid (28.4 mg, 32%); ¹H NMR (500 MHz, DMSO-d₆) δ 7.95 (br s, 1H), 7.91 (d, J=7.8 Hz, 1H), 7.62 (d, J=10.2 Hz, 1H), 7.43-7.55 (m, 4H), 7.18 (d, J=8.3 Hz, 2H), 6.28 (s, 2H), 4.06 (s, 1H), 3.36-3.47 (m, 3H), 3.14 (d, J=8.8 Hz, 1H), 2.96 (br t, J=6.4 Hz, 2H), 2.66 (d, J=8.6 Hz, 1H), 2.54-2.59 (m, 1H), 2.35-2.43 (m, 2H), 1.76 (dt, J=7.7, 3.8 Hz, 1H), 1.37 (t, J=3.9 Hz, 1H), 1.09 (d, J=2.2 Hz, 6H), 0.75 (dd, J=7.8, 3.5 Hz, 1H). LCMS: [M+H]⁺=487.44.

### Example 126: 6-(2-amino-6-fluoro-5-(4-((1S,5R)-3-(2-hydroxy-2-methylpropyl)-3-azabicyclo[3.1.0]hexan-1-yl)phenyl)pyridin-3-yl)-3,4-dihydroisoquinolin-1(2H)-one (I-126)

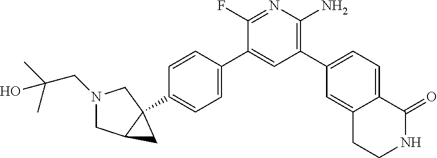 [SMILES: CC(C)(O)CN1C[C@@H]2C[C@]2(c2ccc(-c3cc(-c4ccc5c(c4)CCNC5=O)c(N)nc3F)cc2)C1]

Step 1: 1-((1S,5R)-1-(4-bromophenyl)-3-azabicyclo[3.1.0]hexan-3-yl)-2-methylpropan-2-ol

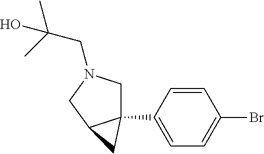 [SMILES: CC(C)(O)CN1C[C@@H]2C[C@]2(c2ccc(Br)cc2)C1]

To a MeOH (5 mL) solution of (1S,5R)-1-(4-bromophenyl)-3-azabicyclo[3.1.0]hexane (0.060 g, 0.25 mmol) was added 1,2-epoxy-2-methylpropane (0.045 mL, 0.50 mmol) at RT and stirring was continued for 21 h. The reaction mixture was concentrated under reduced pressure to afford the product that was used in its entirety in the following step. LCMS: [M+H]⁺=310.24/312.27.

Step 2: 6-(2-amino-6-fluoro-5-(4-((1S,5R)-3-(2-hydroxy-2-methylpropyl)-3-azabicyclo[3.1.0]hexan-1-yl)phenyl)pyridin-3-yl)-3,4-dihydroisoquinolin-1(2H)-one

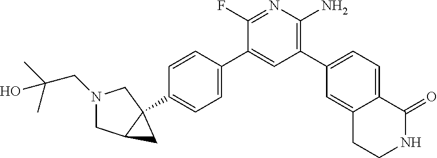 [SMILES: CC(C)(O)CN1C[C@@H]2C[C@]2(c2ccc(-c3cc(-c4ccc5c(c4)CCNC5=O)c(N)nc3F)cc2)C1]

A procedure similar to Example 1, General Method A using 1-((1S,5R)-1-(4-bromophenyl)-3-azabicyclo[3.1.0]hexan-3-yl)-2-methylpropan-2-ol (78 mg, 0.251 mmol, entire material from step 1), 6-(2-amino-6-fluoro-5-(4,4,5,5-tetramethyl-1,3,2-dioxaborolan-2-yl)pyridin-3-yl)-3,4-dihydroisoquinolin-1(2H)-one (116 mg, 0.302 mmol), PdCl₂dppf (28 mg, 0.038 mmol), Cs₂CO₃ (246 mg, 0.754 mmol), DME (4.5 mL) and H₂O (2 mL) afforded the title compound as a beige solid (50 mg, 41% yield for 2 steps). ¹H NMR (500 MHz, DMSO-d₆) δ 7.95 (br s, 1H), 7.91 (d, J=7.8 Hz, 1H), 7.62 (d, J=10.2 Hz, 1H), 7.42-7.50 (m, 4H), 7.18 (d, J=8.44 Hz, 2H), 6.29 (s, 2H), 4.06 (s, 1H), 3.38-3.45 (m, 3H), 3.14 (d, J=8.8 Hz, 1H), 2.96 (t, J=6.5 Hz, 2H), 2.66 (d, J=8.6 Hz, 1H), 2.54-2.59 (m, 1H), 2.35-2.43 (m, 2H), 1.76 (dt, J=7.7, 3.8 Hz, 1H), 1.37 (t, J=3.9 Hz, 1H), 1.05-1.12 (m, 6H), 0.75 (dd, J=7.8, 3.6 Hz, 1H); LCMS: [M+H]⁺=487.44.

### Example 127: 6-(2-amino-5-(4-(1-(2,2-difluoroethyl)-2,5-dihydro-1H-pyrrol-3-yl)phenyl)-6-fluoropyridin-3-yl)-3,4-dihydroisoquinolin-1(2H)-one (I-127)

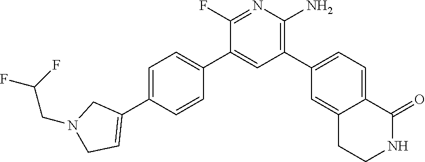 [SMILES: Nc1nc(F)c(-c2ccc(C3=CCN(CC(F)F)C3)cc2)cc1-c1ccc2c(c1)CCNC2=O]

A procedure analogous to that of Example 90, Step 2, by stirring 6-(2-amino-5-(4-(2,5-dihydro-1 H-pyrrol-3-yl)phenyl)-6-fluoropyridin-3-yl)-3,4-dihydroisoquinolin-1(2H)-one-TFA (45 mg, 0.087 mmol) and 2,2-difluoroethyl trifluoromethanesulfonate (0.029 ml, 0.219 mmol) for 3 h at RT. Workup and purification afforded the title compound as a white solid (19 mg, 44%). ¹H NMR (500 MHz, DMSO-d₆) δ 7.98-7.93 (m, 1H), 7.91 (d, J=7.9 Hz, 1H), 7.70-7.66 (m, 1H), 7.58-7.54 (m, 2H), 7.51-7.43 (m, 4H), 6.44-6.31 (m, 3H), 6.25-5.98 (m, 1H), 3.99-3.91 (m, 2H), 3.80-3.70 (m, 2H), 3.45-3.40 (m, 2H), 3.16-3.07 (m, 2H), 3.00-2.91 (m, 2H); LCMS: [M+H]⁺=465.47.

### Example 128 6-(2-amino-6-fluoro-5-(4-(piperidin-4-ylsulfonyl)phenyl)pyridin-3-yl)-3,4-dihydroisoquinolin-1(2H)-one (I-128)

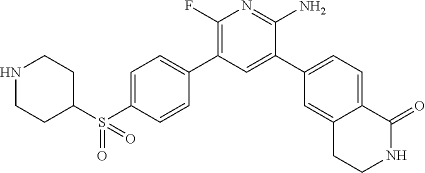 [SMILES: Nc1nc(F)c(-c2ccc(S(=O)(=O)C3CCNCC3)cc2)cc1-c1ccc2c(c1)CCNC2=O]

In a vial was placed tert-butyl 4-((4-(6-amino-2-fluoro-5-(1-oxo-1,2,3,4-tetrahydroisoquinolin-6-yl)pyridin-3-yl)phenyl)sulfonyl)piperidine-1-carboxylate (91 mg, 0.157 mmol), CH₂Cl₂ (5 mL) and TFA (0.012 mL, 0.157 mmol). The flask was mixed by vortexing and then allowed to sit for 2 h, when LCMS indicated complete conversion. The reaction was concentrated and then purified by loading onto a column containing PoraPak RxnCX acidic PE resin, washing (MeOH) and then eluting with 3% NH₄OH in MeOH to provide after concentrating solvent the title compound (26 mg, 34%) as a white solid. ¹H NMR (500 MHz, DMSO-d₆) δ 7.96 (br s, 1H), 7.91 (d, J=7.9 Hz, 1H), 7.89-7.85 (m, 2H), 7.85-7.82 (m, 2H), 7.78 (d, J=10.1 Hz, 1H), 7.50-7.47 (m, 1H), 7.46 (s, 1H), 6.56 (s, 2H), 3.41 (td, J=6.5, 2.7 Hz, 3H), 3.36 (br d, J=3.7 Hz, 1H), 3.01 (br d, J=12.3 Hz, 2H), 2.95 (t, J=6.5 Hz, 2H), 2.49-2.42 (m, 2H), 1.79 (br d, J=11.9 Hz, 2H), 1.41 (qd, J=12.3, 4.0 Hz, 2H); LCMS: [M+H]⁺=481.12.

### Example 129: 6-(2-amino-6-fluoro-5-(1-(1-methylpiperidin-4-yl)-1H-pyrazol-4-yl)pyridin-3-yl)-7-fluoro-3,4-dihydroisoquinolin-1(2H)-one (I-129)

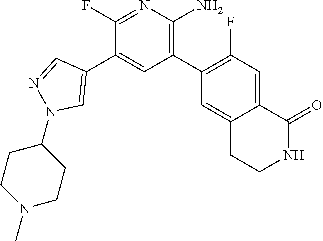 [SMILES: CN1CCC(n2cc(-c3cc(-c4cc5c(cc4F)C(=O)NCC5)c(N)nc3F)cn2)CC1]

Step 1: 3-chloro-6-fluoro-5-(1-(1-methylpiperidin-4-yl)-1H-pyrazol-4-yl)pyridin-2-amine

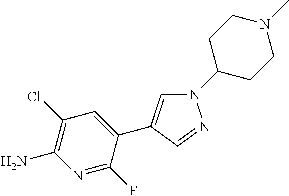 [SMILES: CN1CCC(n2cc(-c3cc(Cl)c(N)nc3F)cn2)CC1]

A sealed and degassed mixture of 5-bromo-3-chloro-6-fluoropyridin-2-amine (150 mg, 0.665 mmol), PdCl₂dppf (49 mg, 0.067 mmol), Cs₂CO₃ (650 mg, 2.0 mmol), 1-methyl-4-(4-(4,4,5,5-tetramethyl-1,3,2-dioxaborolan-2-yl)-1 H-pyrazol-1-yl)piperidine (194 mg, 0.665 mmol), H₂O (5 mL) and DME (10 mL) was heated in a microwave reactor at 90° C. for 1.5 h. The reaction mixture was concentrated and purified by flash chromatography (25 g SiO₂, using MeOH in CH₂Cl₂) to afford the product as a brown solid (71 mg, 32% yield based on purity of 93%). LCMS: [M+H]⁺=310.12.

Step 2: 6-(2-amino-6-fluoro-5-(1-(1-methylpiperidin-4-yl)-1H-pyrazol-4-yl)pyridin-3-yl)-7-fluoro-3,4-dihydroisoquinolin-1(2H)-one

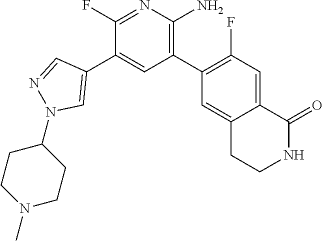 [SMILES: CN1CCC(n2cc(-c3cc(-c4cc5c(cc4F)C(=O)NCC5)c(N)nc3F)cn2)CC1]

A procedure similar to Example 1, General Method A using K₃PO₄ (136 mg, 0.639 mmol), 7-fluoro-6-(4,4,5,5-tetramethyl-1,3,2-dioxaborolan-2-yl)-3,4-dihydroisoquinolin-1(2H)-one (93 mg, 0.32 mmol, preparation described in Example 95, Step 2), 3-chloro-6-fluoro-5-(1-(1-methylpiperidin-4-yl)-1H-pyrazol-4-yl)pyridin-2-amine (71 mg, 0.213 mmol, 93 % purity), XPhos Pd G2 (17 mg, 0.021 mmol), H₂O (6 mL) and MeCN (9 mL) afforded the title compound as a pale yellow solid (53 mg, 55% yield based on purity of 97%). ¹H NMR (500 MHz, DMSO-d₆) δ 8.12 (br s, 1H), 8.09 (s, 1H), 7.82 (d, J=9.9 Hz, 1H), 7.77 (d, J=1.2 Hz, 1H), 7.62 (d, J=10.0 Hz, 1H), 7.40 (d, J=7.0 Hz, 1H), 6.12 (s, 2H), 4.07-4.17 (m, 1H), 3.38-3.45 (m, 2H), 2.93 (br t, J=6.5 Hz, 2H), 2.85 (br d, J=11.4 Hz, 2H), 2.21 (s, 3H), 1.91-2.09 (m, 6H); LCMS [M+H]⁺=439.44.

### Example 130: (S)-6-(2-amino-6-fluoro-5-(4-(2-isopropylmorpholino)-3-(trifluoromethyl)phenyl)pyridin-3-yl)-3,4-dihydroisoquinolin-1(2H)-one (I-130)

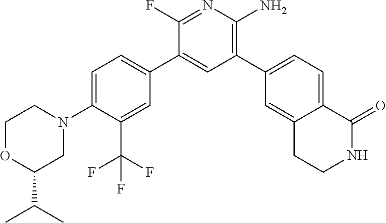 [SMILES: CC(C)[C@H]1CN(c2ccc(-c3cc(-c4ccc5c(c4)CCNC5=O)c(N)nc3F)cc2C(F)(F)F)CCO1]

To a solution of (S)-4-(4-chloro-2-(trifluoromethyl)phenyl)-2-isopropylmorpholine (37.1 mg, 0.121 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (47.3 mg, 0.123 mmol) in 1,4-dioxane (3 mL) was added a solution of K₃PO₄ (79 mg, 0.370 mmol) in water (1 ml) at RT and the reaction mixture was degassed under argon for 15 minutes. Then XPhos Pd G2 (9.71 mg, 0.012 mmol) was added and the reaction mixture was heated at 90° C. for overnight. The reaction mixture was dry loaded onto Celite® and purified by flash chromatography [0-10%, DCM/MeOH] to afford the title compound (11.0 mg, 16%) as a white powder. ¹H NMR (500 MHz, CD₃OD) δ 8.05 (d, J=7.9 Hz, 1H), 7.82-7.75 (m, 2H), 7.66 (d, J=9.9 Hz, 1H), 7.56-7.50 (m, 2H), 7.47 (s, 1H), 3.99-3.93 (m, 1H), 3.77 (dt, J=2.4, 11.0 Hz, 1H), 3.56 (t, J=6.7 Hz, 2H), 3.07 (t, J=6.5 Hz, 2H), 3.04-2.93 (m, 2H), 2.86 (br d, J=11.0 Hz, 1H), 2.67 (t, J=10.5 Hz, 1H), 1.78-1.68 (m, 1H), 1.34-1.28 (m, 1H), 1.00 (d, J=6.7 Hz, 3H), 0.92 (d, J=6.8 Hz, 3H), NH and NH₂ peaks not visible; ¹⁹F NMR (471 MHz, CD₃OD) 5=−61.46 (s, 3F),−77.40 (s, 1F).

### Example 131: 6-(2-amino-5-(4-(4-ethylmorpholin-3-yl)phenyl)-6-fluoropyridin-3-yl)-3,4-dihydroisoquinolin-1(2H)-one (I-131)

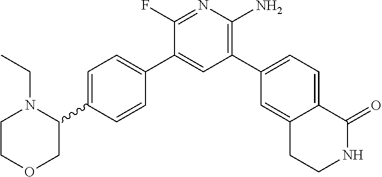 [SMILES: CCN1CCOCC1c1ccc(-c2cc(-c3ccc4c(c3)CCNC4=O)c(N)nc2F)cc1]

Step 1: 6-(2-Amino-6-fluoro-5-(4-(morpholin-3-yl)phenyl)pyridin-3-yl)-3,4-dihydroisoquinolin-1(2H)-one

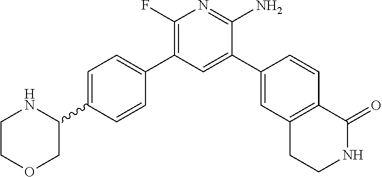 [SMILES: Nc1nc(F)c(-c2ccc(C3COCCN3)cc2)cc1-c1ccc2c(c1)CCNC2=O]

A procedure analogous to that of Example 43, Step 5, by coupling 3-(4-bromophenyl)morpholine hydrochloride (200 mg, 0.718 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (344 mg, 0.897 mmol) afforded the product as a beige solid (166 mg, 53%); LCMS: [M+H]⁺=419.40.

Step 2: 6-(2-amino-5-(4-(4-ethylmorpholin-3-yl)phenyl)-6-fluoropyridin-3-yl)-3,4-dihydroisoquinolin-1(2H)-one

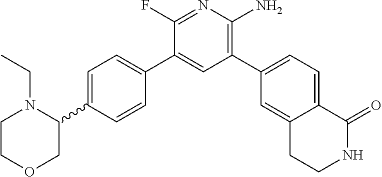 [SMILES: CCN1CCOCC1c1ccc(-c2cc(-c3ccc4c(c3)CCNC4=O)c(N)nc2F)cc1]

A procedure analogous to that of Example 90, Step 2 was used, heating 6-(2-amino-6-fluoro-5-(4-(morpholin-3-yl)phenyl)pyridin-3-yl)-3,4-dihydroisoquinolin-1(2H)-one (32 mg, 0.073 mmol) and bromoethane (0.016 ml, 0.220 mmol) at 80° C. for 6 h. The reaction mixture was partitioned between water (3 mL) and CH₂Cl₂ (3 mL). The layers were separated and the aqueous layer was further extracted with CH₂Cl₂ (x 2). The combined organic extracts was dried and concentrated onto Celite®. Silica gel chromatography (eluting with CH₂Cl₂ containing 0-3% MeOH and 0-0.3% NH₄OH) followed by reverse phase chromatography (C18; 0-40% acetonitrile/water) afforded the title compound as a beige solid (15 mg, 44%). ¹H NMR (500 MHz, DMSO-d₆) δ 7.93-7.99 (m, 1H), 7.89-7.93 (m, 1H), 7.51-7.56 (m, 2 H), 7.46-7.51 (m, 2H), 7.37-7.44 (m, 2H), 6.29-6.45 (m, 2H), 3.85-3.90 (m, 1H), 3.59-3.65 (m, 2H), 3.40-3.45 (m, 2H), 3.18-3.30 (m, 2H), 2.93-3.00 (m, 3H), 2.45-2.50 (m, 1H), 2.20-2.30 (m, 1H), 1.95-2.05 (m, 1H), 0.89-0.94 (m, 3H); LCMS: [M+H]⁺=447.48.

### Example 132: 6-(2-amino-6-fluoro-5-(4-(4-isopropylmorpholin-3-yl)phenyl)pyridin-3-yl)-3,4-dihydroisoquinolin-1(2H)-one (I-132)

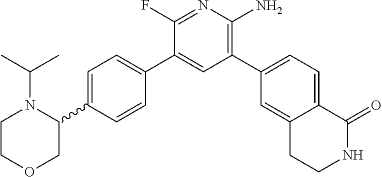 [SMILES: CC(C)N1CCOCC1c1ccc(-c2cc(-c3ccc4c(c3)CCNC4=O)c(N)nc2F)cc1]

A procedure analogous to that of Example 90, Step 2 heating 6-(2-amino-6-fluoro-5-(4-(morpholin-3-yl)phenyl)pyridin-3-yl)-3,4-dihydroisoquinolin-1(2H)-one (30 mg, 0.072 mmol) and 2-iodopropane (0.029 ml, 0.287 mmol) overnight at 80° C. afforded, after workup and purification, the title compound as an off white solid (10.5 mg, 30%). ¹H NMR (500 MHz, DMSO-d₆) δ 7.9-8.0 (m, 1H), 7.9-7.9 (m, 1H), 7.66 (d, J=10.1 Hz, 1H), 7.5-7.5 (m, 2H), 7.5-7.5 (m, 1H), 7.45 (s, 1H), 7.4-7.4 (m, 2H), 6.3-6.4 (m, 2H), 3.8-3.9 (m, 1H), 3.5-3.6 (m, 3H), 3.4-3.4 (m, 2H), 3.2-3.3 (m, 1H), 2.9-3.0 (m, 2H), 2.7-2.8 (m, 2H), 2.4-2.5 (m, 1H), 0.9-1.0 (m, 3H), 0.7-0.8 (m, 3H); LCMS: [M+H]⁺=461.41.

### Example 133: 6-(2-amino-5-(4-(4-(2,2-difluoroethyl)morpholin-3-yl)phenyl)-6-fluoropyridin-3-yl)-3,4-dihydroisoquinolin-1(2H)-one (I-133)

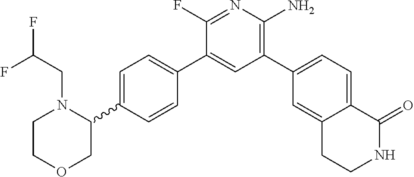 [SMILES: Nc1nc(F)c(-c2ccc(C3COCCN3CC(F)F)cc2)cc1-c1ccc2c(c1)CCNC2=O]

A procedure analogous to that of Example 90, Step 2 using 6-(2-amino-6-fluoro-5-(4-(morpholin-3-yl)phenyl)pyridin-3-yl)-3,4-dihydroisoquinolin-1(2H)-one (30 mg, 0.072 mmol) and 2,2-difluoroethyl trifluoromethanesulfonate (0.038 mL, 0.287 mmol) and heating overnight at 80° C. afforded the title compound as an off white solid (11.5 mg, 32%). ¹H NMR (500 MHz, DMSO-d₆) δ 7.9-8.0 (m, 1H), 7.9-7.9 (m, 1H), 7.6-7.7 (m, 1H), 7.56 (br d, J=7.7 Hz, 2H), 7.5-7.5 (m, 1H), 7.5-7.5 (m, 1H), 7.4-7.5 (m, 2H), 6.3-6.4 (m, 2H), 5.9-6.2 (m, 1H), 3.8-3.9 (m, 1H), 3.6-3.7 (m, 2H), 3.5-3.5 (m, 1H), 3.4-3.4 (m, 2H), 3.3-3.3 (m, 2H), 3.0-3.1 (m, 1H), 2.9-3.0 (m, 2H), 2.6-2.8 (m, 1H), 2.5-2.6 (m, 1H); LCMS: [M+H]⁺=483.52.

### Example 134: 6-(2-amino-6-fluoro-5-(4-(4-(2-methoxyethyl)morpholin-3-yl)phenyl)pyridin-3-yl)-3,4-dihydroisoquinolin-1(2H)-one (I-134)

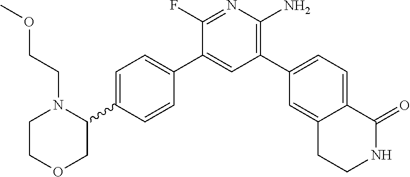 [SMILES: COCCN1CCOCC1c1ccc(-c2cc(-c3ccc4c(c3)CCNC4=O)c(N)nc2F)cc1]

A procedure analogous to that of Example 90, Step 2 using 6-(2-amino-6-fluoro-5-(4-(morpholin-3-yl)phenyl)pyridin-3-yl)-3,4-dihydroisoquinolin-1(2H)-one (30 mg, 0.072 mmol) and 2-bromoethyl methyl ether (0.027 ml, 0.287 mmol) and heating for 5 h at 80° C. afforded the title compound (16 mg, 45%) as a white solid. ¹H NMR (500 MHz, DMSO-d₆) δ 7.9-8.0 (m, 1H), 7.9-7.9 (m, 1H), 7.6-7.7 (m, 1H), 7.5-7.6 (m, 2H), 7.5-7.5 (m, 1H), 7.47 (s, 1H), 7.4-7.4 (m, 2H), 6.3-6.4 (m, 2H), 3.8-3.9 (m, 1H), 3.6-3.7 (m, 2H), 3.4-3.5 (m, 2H), 3.3-3.4 (m, 3H), 3.2-3.3 (m, 1H), 3.1-3.2 (m, 3H), 3.0-3.1 (m, 1H), 2.9-3.0, (m, 2H), 2.6-2.6 (m, 1H), 2.3-2.4 (m, 1H), 2.1-2.2 (m, 1H); LCMS: [M+H]⁺=477.44.

### Example 135: 6-(5-(4-((1R,5S)-3-azabicyclo[3.1.0]hexan-1-yl)phenyl)-2-amino-6-fluoropyridin-3-yl)-3,4-dihydroisoquinolin-1(2H)-one (I-135)

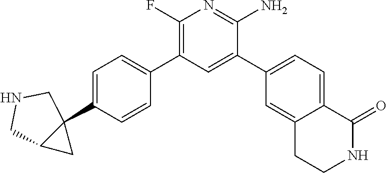 [SMILES: Nc1nc(F)c(-c2ccc([C@]34CNC[C@H]3C4)cc2)cc1-c1ccc2c(c1)CCNC2=O]

A procedure analogous to that of Example 43, Step 5 using (1R,5S)-1-(4-bromophenyl)-3-azabicyclo[3.1.0]hexane (60 mg, 0.252 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (126 mg, 0.328 mmol) afforded the title compound (75 mg, 68%) as a beige solid. ¹H NMR (500 MHz, DMSO-d₆) δ 7.9-8.0 (m, 1H), 7.9-7.9 (m, 1H), 7.6-7.6 (m, 1H), 7.4-7.5 (m, 4H), 7.2-7.3 (m, 2H), 6.2-6.4 (m, 2H), 3.41 (br s, 2H), 3.1-3.2 (m, 1H), 2.9-3.0 (m, 3H), 2.87 (s, 2H), 1.7-1.7 (m, 1H), 1.0-1.0 (m, 1H), 0.8-0.9 (m, 1H), 0.8-0.8 (m, 1H); LCMS: [M+H]⁺=415.58.

### Example 136: (S)-6-(2-amino-6-chloro-5-(4-(2-isopropylmorpholino)phenyl)pyridin-3-yl)-3,4-dihydroisoquinolin-1(2H)-one (I-136)

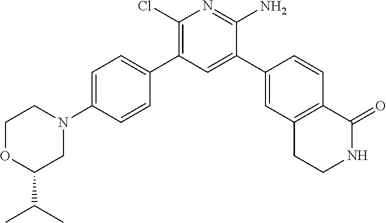 [SMILES: CC(C)[C@H]1CN(c2ccc(-c3cc(-c4ccc5c(c4)CCNC5=O)c(N)nc3Cl)cc2)CCO1]

Step 1: (S)-4-(4-chlorophenyl)-2-isopropylmorpholine

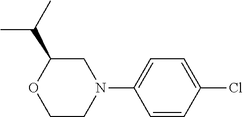 [SMILES: CC(C)[C@H]1CN(c2ccc(Cl)cc2)CCO1]

The procedure followed was analogous to Example 47, Step 1 using 1-chloro-4-iodobenzene (0.500 g, 2.10 mmol), (S)-2-isopropylmorpholine (0.298 g, 2.31 mmol), tris(dibenzylideneacetone)dipalladium (0) (0.058 g, 0.063 mmol), 4,5-bis(diphenylphosphino)-9,9-dimethylxanthene (0.109 g, 0.189 mmol) and Cs₂CO₃ (2.05 g, 6.29 mmol). Workup and purification afforded the product (404 mg, 80%). LCMS: [M+H]⁺=240.03.

Step 2: (S)-2-isopropyl-4-(4-(4,4,5,5-tetramethyl-1,3,2-dioxaborolan-2-yl)phenyl)morpholine

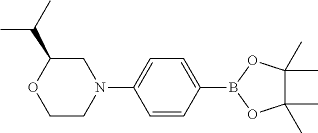 [SMILES: CC(C)[C@H]1CN(c2ccc(B3OC(C)(C)C(C)(C)O3)cc2)CCO1]

A procedure analogous to Example 47, Step 2 using (S)-4-(4-chlorophenyl)-2-isopropylmorpholine (0.404 g, 1.67 mmol), bis(pinacolato)diboron (0.535 g, 2.106 mmol), KOAc (477 mg, 3.37 mmol) and XPhos Pd G2 (0.099 g, 0.126 mmol) afforded the product (1.44 mmol, 85%). LCMS: [M+H]=332.08.

Step 3: (S)-6-(2-Amino-6-chloro-5-(4-(2-isopropylmorpholino)phenyl)pyridin-3-yl)-3,4-dihydroisoquinolin-1(2H)-one

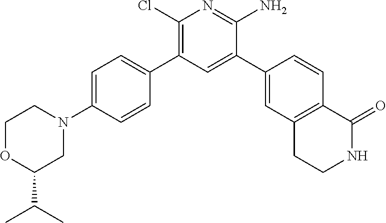 [SMILES: CC(C)[C@H]1CN(c2ccc(-c3cc(-c4ccc5c(c4)CCNC5=O)c(N)nc3Cl)cc2)CCO1]

A procedure analogous to that of Example 43, Step 5 was used, heating 6-(2-amino-5-bromo-6-chloropyridin-3-yl)-3,4-dihydroisoquinolin-1(2H)-one (35 mg, 0.099 mmol) and (S)-2-isopropyl-4-(4-(4,4,5,5-tetramethyl-1,3,2-dioxaborolan-2-yl)phenylmorpholine (42.7 mg, 0.129 mmol) at 90° C. for 1.75 h. The reaction mixture was partitioned between brine and EtOAc. The organic phase was separated and aqueous phase was further extracted with EtOAc (2×3 mL). The combined organic phase was dried and concentrated onto Celite®. Silica gel chromatography (eluting with 0-3% MeOH/DCM), followed by reverse phase chromatography (C18; 0-40% acetonitrile/water), afforded the title compound as a white solid (37.5 mg, 75%). ¹H NMR (500 MHz, DMSO-d₆) δ 7.99-7.93 (m, 1H), 7.92-7.87 (m, 1H), 7.51-7.43 (m, 2H), 7.39-7.29 (m, 3H), 7.07-6.96 (m, 2H), 6.34-6.13 (m, 2H), 4.02-3.94 (m, 1H), 3.65-3.58 (m, 2H), 3.57-3.51 (m, 1H), 3.44-3.38 (m, 2H), 3.28-3.21 (m, 1H), 2.98-2.91 (m, 2H), 2.74-2.65 (m, 1H), 2.49-2.42 (m, 1H), 1.80-1.71 (m, 1H), 1.00-0.93 (m, 6H); LCMS: [M+H]⁺=477.50.

### Example 137: (R)-6-(2-amino-6-chloro-5-(2-fluoro-4-(2-isopropylmorpholino)phenyl)pyridin-3-yl)-3,4-dihydroisoquinolin-1(2H)-one (I-137)

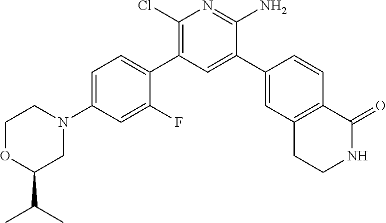 [SMILES: CC(C)[C@@H]1CN(c2ccc(-c3cc(-c4ccc5c(c4)CCNC5=O)c(N)nc3Cl)c(F)c2)CCO1]

Step 1: (R)-4-(4-chloro-3-fluorophenyl)-2-isopropylmorpholine

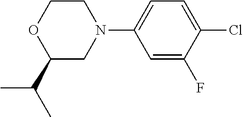 [SMILES: CC(C)[C@@H]1CN(c2ccc(Cl)c(F)c2)CCO1]

A 30 mL vial was charged with 4-bromo-1-chloro-2-fluorobenzene (0.15 g, 0.72 mmol), (R)-2-isopropylmorpholine (0.10 g, 0.79 mmol), tris(dibenzylideneacetone)dipalladium (0) (0.020 g, 0.021 mmol), 4,5-bis(diphenylphosphino)-9,9-dimethylxanthene (0.037 g, 0.064 mmol) and Cs₂CO₃ (0.70 g, 2.1 mmol). The vial was sealed with a cap and septum and then the reaction vial was evacuated and backfilled with nitrogen. Toluene (2.5 mL) was added and the reaction vial was evacuated and backfilled with nitrogen an additional time. The reaction was heated conventionally at 100° C. for 18 h. The reaction mixture was cooled to RT and partitioned between EtOAc and water. The layers were separated and the aqueous layer was extracted with additional EtOAc (x2). The combined organic extracts were dried and concentrated onto Celite®. Flash chromatography (1-15% EtOAc/hexanes) afforded the product (0.18 g, 99%). LCMS: [M+H]⁺=258.2.

Step 2: (R)-4-(3-fluoro-4-(4,4,5,5-tetramethyl-1,3,2-dioxaborolan-2-yl)phenyl)-2-isopropylmorpholine

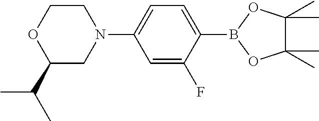 [SMILES: CC(C)[C@@H]1CN(c2ccc(B3OC(C)(C)C(C)(C)O3)c(F)c2)CCO1]

A 30 mL vial was charged with (R)-4-(4-chloro-3-fluorophenyl)-2-isopropylmorpholine (0.67 g, 2.6 mmol), bis(pinacolato)diboron (0.83 g, 3.3 mmol), KOAc (0.51 g, 5.2 mmol) and XPhos Pd G2 (0.15 g, 0.20 mmol). The vial was sealed with a cap and septum and then the reaction vial was evacuated and backfilled with N2. 1,4-Dioxane (8 mL) was added and the reaction vial was evacuated and backfilled with nitrogen an additional time. The reaction was heated at 90° C. for 18 h. After cooling to RT the reaction mixture was concentrated directly onto Celite® and purified by flash chromatography (1-15% EtOAc/hexanes) to afford the product (0.92 g, quantitative yield). LCMS: [M+H]⁺=350.2.

Step 3: (R)-6-(2-amino-6-chloro-5-(2-fluoro-4-(2-isopropylmorpholino)phenyl)pyridin-3-yl)-3,4-dihydroisoquinolin-1(2H)-one

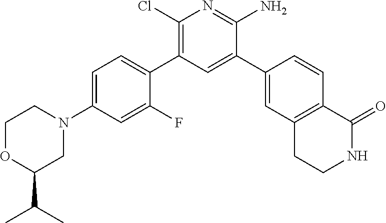 [SMILES: CC(C)[C@@H]1CN(c2ccc(-c3cc(-c4ccc5c(c4)CCNC5=O)c(N)nc3Cl)c(F)c2)CCO1]

A procedure analogous to that of Example 43, Step 5 using 6-(2-amino-5-bromo-6-chloropyridin-3-yl)-3,4-dihydroisoquinolin-1(2H)-one (35 mg, 0.099 mmol) and (R)-4-(3-fluoro-4-(4,4,5,5-tetramethyl-1,3,2-dioxaborolan-2-yl)phenyl)-2-isopropylmorpholine (45.1 mg, 0.129 mmol) afforded the title compound (15.5 mg, 30%) as a pale yellow solid. ¹H NMR (500 MHz, DMSO-d₆) δ 7.9-8.0 (m, 1H), 7.9-7.9 (m, 1H), 7.4-7.5 (m, 2H), 7.3-7.4 (m, 1H), 7.2-7.3 (m, 1H), 6.8-6.9 (m, 2H), 6.3-6.4 (m, 2H), 3.9-4.0 (m, 1H), 3.6-3.7 (m, 3H), 3.4-3.4 (m, 2H), 3.3-3.3 (m, 1H), 3.2-3.3 (m, 1H), 2.9-3.0 (m, 2H), 2.7-2.8 (m, 1H), 1.7-1.8 (m, 1H), 0.9-1.0 (m, 6H); LCMS: [M+H]⁺=495.49.

### Example 138: 6-(2-amino-6-fluoro-5-(4-(1-(2-hydroxyethyl)-1,2,3,6-tetrahydropyridin-4-yl)phenyl)pyridin-3-yl)-3,4-dihydroisoquinolin-1(2H)-one (I-138)

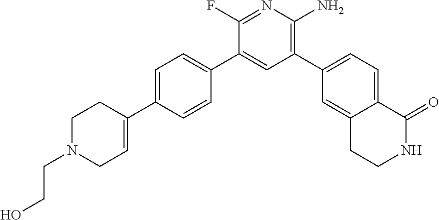 [SMILES: Nc1nc(F)c(-c2ccc(C3=CCN(CCO)CC3)cc2)cc1-c1ccc2c(c1)CCNC2=O]

A procedure analogous to that of Example 53, Step 1, by stirring 6-(2-amino-6-fluoro-5-(4-(1,2,3,6-tetrahydropyridin-4-yl)phenyl)pyridin-3-yl)-3,4-dihydroisoquinolin-1(2H)-one TFA (45 mg, 0.085 mmol) and 2-bromoethanol, 95% (0.018 ml, 0.255 mmol) for 16 h at RT afforded the title compound (7.5 mg, 18%) as a yellow solid. ¹H NMR (500 MHz, DMSO-d₆) δ 7.9-8.0 (m, 1H), 7.9-7.9 (m, 1H), 7.6-7.7 (m, 1H), 7.5-7.6 (m, 2H), 7.4-7.5 (m, 4H), 6.3-6.4 (m, 2H), 6.2-6.2 (m, 1H), 4.4-4.5 (m, 1H), 3.5-3.6 (m, 2H), 3.4-3.4 (m, 2H), 3.3-3.4 (m, 2H), 3.3-3.3 (m, 2H), 3.1-3.2 (m, 2H), 2.9-3.0 (m, 2H), 2.6-2.7 (m, 2H); LCMS: [M+H]⁺=459.45.

### Example 139: 6-(2-amino-6-fluoro-5-(4-(1-(2-methoxyethyl)-1,2,3,6-tetrahydropyridin-4-yl)phenyl)pyridin-3-yl)-3,4-dihydroisoquinolin-1(2H)-one (I-139)

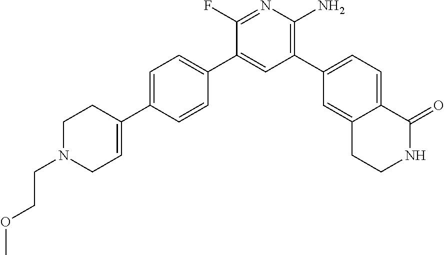 [SMILES: COCCN1CC=C(c2ccc(-c3cc(-c4ccc5c(c4)CCNC5=O)c(N)nc3F)cc2)CC1]

A procedure analogous to that of Example 90, Step 2, by stirring 6-(2-amino-6-fluoro-5-(4-(1,2,3,6-tetrahydropyridin-4-yl)phenyl)pyridin-3-yl)-3,4-dihydroisoquinolin-1(2H)-one.TFA (45 mg, 0.085 mmol) and 2-bromoethyl methyl ether (0.024 mL, 0.255 mmol) overnight at RT afforded the title compound as a yellow solid (25.5 mg, 60%). ¹H NMR (500 MHz, DMSO-d₆) δ 7.98-7.92 (m, 1H), 7.92-7.87 (m, 1H), 7.67-7.63 (m, 1H), 7.55-7.50 (m, 2H), 7.50-7.42 (m, 4H), 6.38-6.26 (m, 2H), 6.23-6.14 (m, 1H), 3.51-3.47 (m, 2H), 3.43-3.39 (m, 2H), 3.26-3.22 (m, 3H), 3.15-3.08 (m, 2H), 2.95 (br t, J=6.4 Hz, 2H), 2.72-2.64 (m, 2H), 2.62-2.55 (m, 2H), 2.49-2.44 (m, 2H); LCMS: [M+H]⁺=473.49.

### Example 140: 6-(2-amino-6-fluoro-5-(4-((1R,5S)-3-(2-methoxyethyl)-3-azabicyclo[3.1.0]hexan-1-yl)phenyl)pyridin-3-yl)-3,4-dihydroisoquinolin-1(2H)-one (I-140)

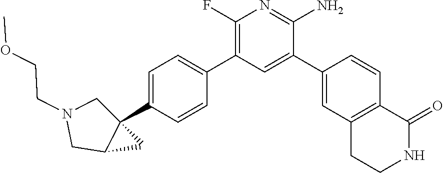 [SMILES: COCCN1C[C@H]2C[C@@]2(c2ccc(-c3cc(-c4ccc5c(c4)CCNC5=O)c(N)nc3F)cc2)C1]

A procedure analogous to that of Example 43, Step 5, by stirring 6-(5-(4-((1R,5S)-3-azabicyclo[3.1.0]hexan-1-yl)phenyl)-2-amino-6-fluoropyridin-3-yl)-3,4-dihydroisoquinolin-1(2H)-one (60 mg, 0.145 mmol) and 2-bromoethyl methyl ether (0.041 mL, 0.434 mmol) overnight at RT afforded the title compound as an off white solid (38.5 mg, 54%). ¹H NMR (500 MHz, DMSO-d₆) δ 7.9-8.0 (m, 1H), 7.9-7.9 (m, 1H), 7.6-7.6 (m, 1H), 7.4-7.5 (m, 4H), 7.1-7.2 (m, 2H), 6.2-6.3 (m, 2H), 3.4-3.5 (m, 5H), 3.24 (s, 3H), 3.0-3.1 (m, 1H), 2.9-3.0 (m, 2H), 2.6-2.7 (m, 2H), 2.6-2.6 (m, 1H), 2.4-2.5 (m, 1H), 1.8-1.8 (m, 1H), 1.3-1.3 (m, 1H), 0.7-0.8 (m, 1H); LCMS: [M+H]⁺=473.56.

### Example 141: 6-(2-amino-6-fluoro-5-(4-((1R,5S)-3-isopropyl-3-azabicyclo[3.1.0]hexan-1-yl)phenyl)pyridin-3-yl)-7-fluoro-3,4-dihydroisoquinolin-1(2H)-one (I-141)

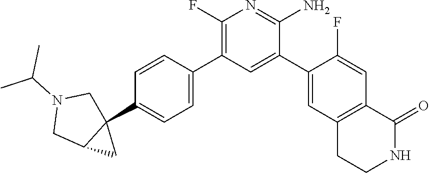 [SMILES: CC(C)N1C[C@H]2C[C@@]2(c2ccc(-c3cc(-c4cc5c(cc4F)C(=O)NCC5)c(N)nc3F)cc2)C1]

Step 1: 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one

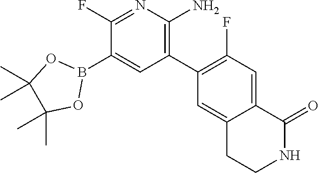 [SMILES: CC1(C)OB(c2cc(-c3cc4c(cc3F)C(=O)NCC4)c(N)nc2F)OC1(C)C]

A degassed mixture of KOAc (248 mg, 2.52 mmol), 6-(2-amino-5-bromo-6-fluoropyridin-3-yl)-7-fluoro-3,4-dihydroisoquinolin-1(2H)-one (266 mg, 0.721 mmol, 96% purity), Pd₂dba₃ (40 mg, 0.043 mmol), dicyclohexyl[2-(2,4,6-triisopropylphenyl)phenyl]phosphane (34 mg, 0.072 mmol) and B₂pin₂ (549 mg, 2.16 mmol) in anh. PhMe (10 mL) was heated sealed at 110° C. for 3 h (˜60% conversion by LCMS). The solvent was removed under reduced pressure and the vacuum-dried solid residue that was mixed with B₂pin₂ (549 mg, 2.16 mmol), PdCl₂dppf (53 mg, 0.072 mmol), B₂pin₂ (549 mg, 2.16 mmol) and 1,4-dioxane (15 mL). The reaction was degassed than heated in an oil bath at 100° C. for 4 h (complete conversion). The resulting crude product was used as the 1,4-dioxane solution in the following step. LCMS: [M+H]⁺=402.42.

Step 2: 6-(2-amino-6-fluoro-5-(4-((1R,5S)-3-isopropyl-3-azabicyclo[3.1.0]hexan-1-yl)phenyl)pyridin-3-yl)-7-fluoro-3,4-dihydroisoquinolin-1(2H)-one

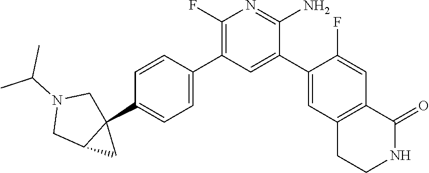 [SMILES: CC(C)N1C[C@H]2C[C@@]2(c2ccc(-c3cc(-c4cc5c(cc4F)C(=O)NCC5)c(N)nc3F)cc2)C1]

A method similar to Example 1, General Method A, heating a sealed and degassed mixture of (1R,5S)-1-(4-bromophenyl)-3-isopropyl-3-azabicyclo[3.1.0]hexane (70 mg, 0.250 mmol), 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (120 mg, 6.23 mL, 0.300 mmol, 19.3 mg/mL) 1,4-dioxane (6.2 mL), PdCl₂dppf (18 mg, 0.025 mmol), Cs₂CO₃ (244 mg, 0.749 mmol) and H₂O (2.0 mL) in a microwave reactor at 90° C. for 1 h afforded the title compound (43 mg, 36%) as a white solid. ¹H NMR (500 MHz, DMSO-d₆) δ 8.04 (br s, 1H), 7.56 (br d, J=3.3 Hz, 1H), 7.52-7.55 (m, 1H), 7.33-7.41 (m, 3H), 7.13 (br d, J=7.6 Hz, 2H), 6.22 (s, 2H), 3.31-3.37 (m, 3H), 2.92-3.09 (m, 1H), 2.85 (br t, J=6.5 Hz, 2H), 2.45-2.68 (m, 2H), 2.25-2.41 (m, 1H), 1.77 (br s, 1H), 1.24 (br s, 1H), 0.98 (br s, 6H), 0.70 (br s, 1H). LCMS: [M+H]⁺=475.41.

### Example 142: 6-(2-amino-6-fluoro-5-(4-((1S,5R)-3-isopropyl-3-azabicyclo[3.1.0]hexan-1-yl)phenyl)pyridin-3-yl)-7-fluoro-3,4-dihydroisoquinolin-1(2H)-one (I-142)

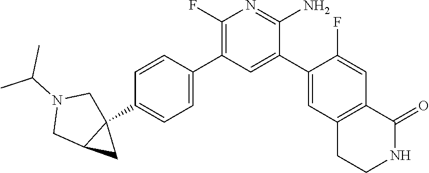 [SMILES: CC(C)N1C[C@@H]2C[C@]2(c2ccc(-c3cc(-c4cc5c(cc4F)C(=O)NCC5)c(N)nc3F)cc2)C1]

A method similar to Example 1, General Method A, heating a sealed and degasssed mixture of (1 S,5R)-1-(4-bromophenyl)-3-isopropyl-3-azabicyclo[3.1.0]hexane (84 mg, 0.249 mmol, 83% purity), 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (6.21 mL, 0.299 mmol, 19.3 mg/mL), 1,4-dioxane (6.2 mL), PdCl₂dppf (18.21 mg, 0.025 mmol), Cs₂CO₃ (243 mg, 0.746 mmol) and H₂O (2.0 mL) in a microwave reactor at 90° C. for 1 h afforded the title compound as an off white solid (49 mg, 41% yield, based on the purity of 99%). ¹H NMR (500 MHz, DMSO-d₆) δ 8.11 (br s, 1H), 7.63 (br d, J=3.6 Hz, 1H), 7.60-7.62 (m, 1H), 7.38-7.49 (m, 3H), 7.21 (br d, J=8.0 Hz, 2H), 6.30 (s, 2H), 3.41 (td, J=6.4, 2.5 Hz, 3H), 3.02-3.20 (m, 1H), 2.93 (br t, J=6.4 Hz, 2H), 2.55-2.74 (m, 2H), 2.34-2.46 (m, 1H), 1.61-1.94 (m, 1H), 1.31 (br s, 1H), 0.97-1.15 (m, 6H), 0.72-0.83 (m, 1H); LCMS: [M+H]⁺=475.41.

### Example 143: 6-(2-amino-6-fluoro-5-(4-((1R,5S)-3-isobutyryl-3-azabicyclo[3.1.0]hexan-1-yl)phenyl)pyridin-3-yl)-3,4-dihydroisoquinolin-1(2H)-one (I-143)

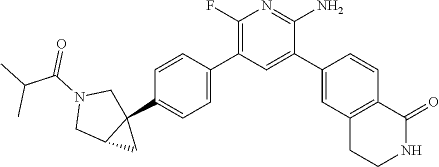 [SMILES: CC(C)C(=O)N1C[C@H]2C[C@@]2(c2ccc(-c3cc(-c4ccc5c(c4)CCNC5=O)c(N)nc3F)cc2)C1]

Step 1: 1-((1R,5S)-1-(4-bromophenyl)-3-azabicyclo[3.1.0]hexan-3-yl)-2-methylpropan-1-one

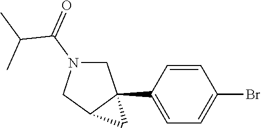 [SMILES: CC(C)C(=O)N1C[C@H]2C[C@@]2(c2ccc(Br)cc2)C1]

To a stirred solution of (1R,5S)-1-(4-bromophenyl)-3-azabicyclo[3.1.0]hexane (92 mg, 0.39 mmol) in anh. CH₂Cl₂ (20 mL) was added isobutyryl chloride (0.040 mL, 0.39 mmol) dropwise at 0° C. The reaction was stirred with cooling and then allowed slowly to warm up to RT. The stirring was continued at RT for 16 h, then 37% aq NH₄OH (˜1 mL) was added at RT. After stirring for 1 h, aq saturated aqueous NaHCO₃ was added (4 mL). The aqueous layer was separated and extracted with CH₂Cl₂. Combined organics were dried (Na₂SO₄), concentrated and dried in vacuo to afford the product as an orange gum (136 mg, quant) that was used without further purification. LCMS: [M+H]=308.28/310.09.

Step 2: 6-(2-amino-6-fluoro-5-(4-((1R,5S)-3-isobutyryl-3-azabicyclo[3.1.0]hexan-1-yl)phenyl)pyridin-3-yl)-3,4-dihydroisoquinolin-1(2H)-one

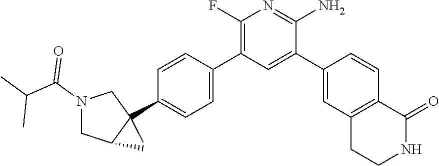

A procedure similar to Example 1, General Method A using 6-(2-amino-6-fluoro-5-(4,4,5,5-tetramethyl-1,3,2-dioxaborolan-2-yl)pyridin-3-yl)-3,4-dihydroisoquinolin-1(2H)-one (71 mg, 0.19 mmol), 1-((1R,5S)-1-(4-bromophenyl)-3-azabicyclo[3.1.0]hexan-3-yl)-2-methylpropan-1-one (60 mg, 0.19 mmol), PdCl₂dppf (13 mg, 0.018 mmol), Cs₂CO₃ (173 mg, 0.531 mmol), DME (4 mL) and H₂O (2 mL) afforded the title compound as a beige solid (19 mg, 22%). ¹H NMR (500 MHz, DMSO-d₆) δ 7.95 (br s, 1H), 7.91 (br d, J=7.8 Hz, 1H), 7.64 (br d, J=10.0 Hz, 1H), 7.41-7.59 (m, 4H), 7.25-7.36 (m, 2H), 6.31 (br s, 2H), 4.04-4.23 (m, 1H), 3.70-3.86 (m, 2H), 3.49 (br d, J=11.5 Hz, 1H), 3.38-3.44 (m, 2H), 2.96 (br t, J=6.1 Hz, 2H), 2.57-2.77 (m, 1H), 1.89-2.16 (m, 1H), 1.10 (br d, J=5.4 Hz, 1H), 0.97-1.05 (m, 6H), 0.73-0.88 (m, 1H); LCMS: [M+H]⁺=485.49.

### Example 144: 6-(2-amino-5-(4-(((2S,6S)-2,6-dimethylpiperidin-4-yl)oxy)phenyl)-6-fluoropyridin-3-yl)-3,4-dihydroisoquinolin-1(2H)-one (I-144)

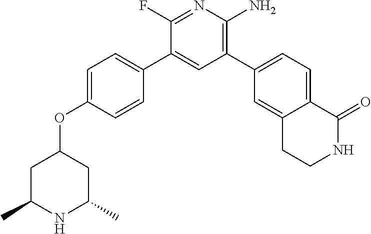 [SMILES: C[C@H]1CC(Oc2ccc(-c3cc(-c4ccc5c(c4)CCNC5=O)c(N)nc3F)cc2)C[C@H](C)N1]

Step 1: tert-butyl (2S,6S)-4-(4-bromophenoxy)-2,6-dimethylpiperidine-1-carboxylate

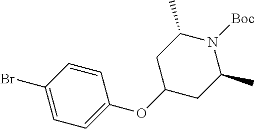 [SMILES: C[C@H]1CC(Oc2ccc(Br)cc2)C[C@H](C)N1C(=O)OC(C)(C)C]

To a solution of 4-bromophenol (646 mg, 3.74 mmol), tert-butyl (2R,6R)-4-hydroxy-2,6-dimethylpiperidine-1-carboxylate (504 mg, 2.198 mmol) and triphenylphosphine (634 mg, 2.42 mmol) in THF (10 mL) was added diisopropyl azodicarboxylate (0.519 ml, 2.64 mmol) slowly. The mixture was allowed to stir in a sealed flask at 50° C. for 3 d. The reaction mixture was dry loaded onto Celite® and purified by flash chromatography (0-20%, hexanes/EtOAc) to afford the a mixture of the desired product (444.4 mg, 53%) and 4-bromophenol with a ratio of 1:1 and as a slightly yellow oil which was used for the next step. LCMS: [M-tBu+H]⁺=328.32.

Step 2: tert-butyl (2S,6S)-4-(4-(6-amino-2-fluoro-5-(1-oxo-1,2,3,4-tetrahydroisoquinolin-6-yl)pyridin-3-yl)phenoxy)-2,6-dimethylpiperidine-1-carboxylate

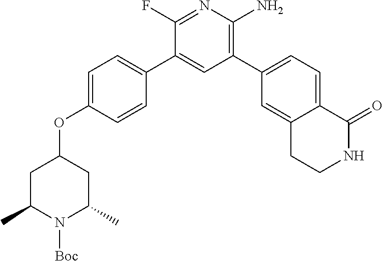 [SMILES: C[C@H]1CC(Oc2ccc(-c3cc(-c4ccc5c(c4)CCNC5=O)c(N)nc3F)cc2)C[C@H](C)N1C(=O)OC(C)(C)C]

A procedure similar to Example 43, Step 5 using 6-(2-amino-6-fluoro-5-(4,4,5,5-tetramethyl-1,3,2-dioxaborolan-2-yl)pyridin-3-yl)-3,4-dihydroisoquinolin-1(2H)-one (143 mg, 0.373 mmol) and the mixture of tert-butyl (2S,6S)-4-(4-bromophenoxy)-2,6-dimethylpiperidine-1-carboxylate (186 mg, 0.485 mmol) and 4-bromophenol with a ratio of 1:1 in 1,4-dioxane (3 ml) and K₃PO₄ (238 mg, 1.12 mmol) in water (1 mL) and XPhos Pd G2 (29.4 mg, 0.037 mmol) afforded the product (60.4 mg, 29%) as a beige powder which was used for the next step. LCMS: [M+H]⁺=561.40.

Step 3: rac-6-(2-amino-5-(4-(((2R,6R)-2,6-dimethylpiperidin-4-yl)oxy)phenyl)-6-fluoropyridin-3-yl)-3,4-dihydroisoquinolin-1(2H)-one

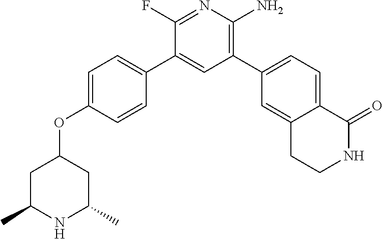 [SMILES: C[C@H]1CC(Oc2ccc(-c3cc(-c4ccc5c(c4)CCNC5=O)c(N)nc3F)cc2)C[C@H](C)N1]

To a solution of tert-butyl (2S,6S)-4-(4-(6-amino-2-fluoro-5-(1-oxo-1,2,3,4-tetrahydroisoquinolin-6-yl)pyridin-3-yl)phenoxy)-2,6-dimethylpiperidine-1-carboxylate (60.4 mg, 0.108 mmol) in CH₂Cl₂ (4 mL) was added TFA (2 mL, 26.1 mmol) at RT and the reaction mixture was stirred for 30 min. The solvents were removed and the crude mixture was dissolved in MeOH and passed through a PoraPak Rxn CX (15cc-2g) cartridge. The cartridge was rinsed with MeOH, and the product was released using 20 mL of a solution of ammonia in MeOH (3N) to afford after removing solvent the title compound (46.5 mg, 89%) as a beige powder. ¹H NMR (500 MHz, DMSO-d₆) δ 7.94 (br s, 1H), 7.90 (d, J=7.8 Hz, 1H), 7.59 (d, J=10.1 Hz, 1H), 7.49-7.41 (m, 4H), 6.98 (d, J=8.8 Hz, 2H), 6.22 (s, 2H), 4.65 (br s, 1H), 3.43-3.38 (m, 2H), 3.11-3.02 (m, 1H), 2.95 (br t, J=6.4 Hz, 2H), 2.04-1.98 (m, 1H), 1.80 (br s, 1H), 1.60-1.52 (m, 1H), 1.17 (d, J=7.0 Hz, 3H), 1.14-1.10 (m, 1H), 1.04 (d, J=6.4 Hz, 3H), 2H under the water peak; LCMS: [M+H]⁺=461.49.

### Example 145: 6-(2-amino-6-fluoro-5-(7-(piperidin-4-yloxy)benzo[d][1,3]dioxol-4-yl)pyridin-3-yl)-3,4-dihydroisoquinolin-1(2H)-one (I-145)

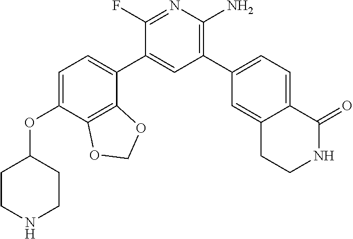 [SMILES: Nc1nc(F)c(-c2ccc(OC3CCNCC3)c3c2OCO3)cc1-c1ccc2c(c1)CCNC2=O]

Step 1: tert-butyl 4-((7-bromobenzo[d][1,3]dioxol-4-yl)oxy)piperidine-1-carboxylate

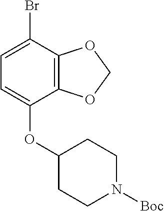 [SMILES: CC(C)(C)OC(=O)N1CCC(Oc2ccc(Br)c3c2OCO3)CC1]

To a solution of 7-bromobenzo[d][1,3]dioxol-4-ol (379.7 mg, 1.75 mmol), tert-butyl 4-hydroxy-1-piperidinecarboxylate (352 mg, 1.75 mmol) and triphenylphosphine (459 mg, 1.75 mmol) in anhydrous THF (10 mL) was added diisopropyl azodicarboxylate (0.344 mL, 1.75 mmol) slowly. The mixture was allowed to stir in a sealed flask at 50° C. for overnight. The reaction mixture was dry loaded onto Celite® and purified by flash chromatography (0-30%, hexanes/EtOAc) to afford the product (374.9 mg, 51%) as a colorless oil which solidified overtime. LCMS: [M-tBu+H]⁺=344.12.

Step 2: tert-butyl 4-((7-(6-amino-2-fluoro-5-(1-oxo-1,2,3,4-tetrahydroisoquinolin-6-yl)pyridin-3-yl)benzo[d][1,3]dioxol-4-yl)oxy)piperidine-1-carboxylate

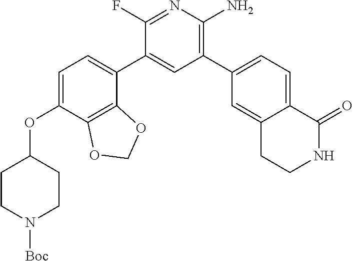 [SMILES: CC(C)(C)OC(=O)N1CCC(Oc2ccc(-c3cc(-c4ccc5c(c4)CCNC5=O)c(N)nc3F)c3c2OCO3)CC1]

A procedure similar to Example 43, Step 5 using tert-butyl 4-((7-bromobenzo[d][1,3]dioxol-4-yl)oxy)piperidine-1-carboxylate (177 mg, 0.441 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (130 mg, 0.339 mmol), 1,4-dioxane (3 ml), K₃PO₄ (216 mg, 1.018 mmol), water (1 mL) and XPhos Pd G2 (26.7 mg, 0.034 mmol) afforded the product (98 mg, 50%) as a beige powder which was used for the next step. LCMS: [M+H]⁺=577.50.

Step 3: 6-(2-amino-6-fluoro-5-(7-(piperidin-4-yloxy)benzo[d][1,3]dioxol-4-yl)pyridin-3-yl)-3,4-dihydroisoquinolin-1(2H)-one

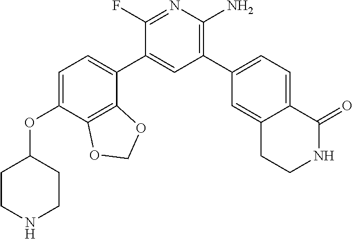 [SMILES: Nc1nc(F)c(-c2ccc(OC3CCNCC3)c3c2OCO3)cc1-c1ccc2c(c1)CCNC2=O]

A procedure similar to Example 144, Step 3 using tert-butyl 4-((7-(6-amino-2-fluoro-5-(1-oxo-1,2,3,4-tetrahydroisoquinolin-6-yl)pyridin-3-yl)benzo[d][1,3]dioxol-4-yl)oxy)piperidine-1-carboxylate (97.9 mg, 0.170 mmol) and TFA (2 mL, 26.1 mmol) in CH₂Cl₂ (4 mL) at RT afforded the title compound (73.4 mg, 86%) as a slightly yellow powder. ¹H NMR (500 MHz, DMSO-d₆) δ 7.93 (br s, 1H), 7.90 (d, J=7.8 Hz, 1H), 7.58 (d, J=9.7 Hz, 1H), 7.40 (s, 2H), 6.88 (d, J=8.1 Hz, 1H), 6.70 (d, J=8.9 Hz, 1H), 6.29 (s, 2H), 5.99 (s, 2H), 4.48-4.40 (m, 1H), 3.44-3.37 (m, 2H), 3.00-2.91 (m, 5H), 2.59-2.52 (m, 2H), 1.95-1.86 (m, 2H), 1.53-1.42 (m, 2H); LCMS: [M+H]⁺=477.44.

### Example 146: 6-(2-amino-5-(4-(azetidin-3-yloxy)phenyl)-6-fluoropyridin-3-yl)-3,4-dihydroisoquinolin-1(2H)-one (I-146)

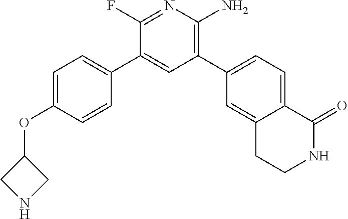 [SMILES: Nc1nc(F)c(-c2ccc(OC3CNC3)cc2)cc1-c1ccc2c(c1)CCNC2=O]

Step 1: tert-butyl 3-(4-(6-amino-2-fluoro-5-(1-oxo-1,2,3,4-tetrahydroisoquinolin-6-yl)pyridin-3-yl)phenoxy)azetidine-1-carboxylate

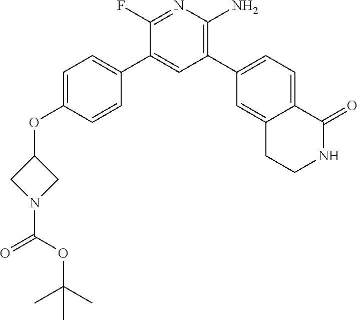 [SMILES: CC(C)(C)OC(=O)N1CC(Oc2ccc(-c3cc(-c4ccc5c(c4)CCNC5=O)c(N)nc3F)cc2)C1]

A procedure analogous to Example 43, Step 5 using 6-(2-amino-6-fluoro-5-(4,4,5,5-tetramethyl-1,3,2-dioxaborolan-2-yl)pyridin-3-yl)-3,4-dihydroisoquinolin-1(2H)-one (133 mg, 0.347 mmol) and tert-butyl 3-(4-bromophenoxy)azetidine-1-carboxylate (137 mg, 0.416 mmol), 1,4-dioxane (3 ml), K₃PO₄ (221 mg, 1.04 mmol), water (1 mL) and XPhos Pd G2 (27.3 mg, 0.035 mmol) afforded the product (43 mg, 25%) as a beige powder which was used for the next step. LCMS: [M+H]⁺=505.50.

Step 2: 6-(2-amino-5-(4-(azetidin-3-yloxy)phenyl)-6-fluoropyridin-3-yl)-3,4-dihydroisoquinolin-1(2H)-one

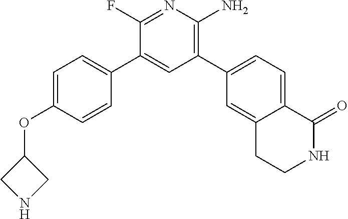 [SMILES: Nc1nc(F)c(-c2ccc(OC3CNC3)cc2)cc1-c1ccc2c(c1)CCNC2=O]

A procedure similar to Example 144, Step 3 using tert-butyl 3-(4-(6-amino-2-fluoro-5-(1-oxo-1,2,3,4-tetrahydroisoquinolin-6-yl)pyridin-3-yl)phenoxy)azetidine-1-carboxylate (43.1 mg, 0.085 mmol), TFA (2 mL, 26.1 mmol) in CH₂Cl₂ (2 mL, 26.1 mmol) afforded the title compound (29.2 mg, 80%) as a beige powder. ¹H NMR (500 MHz, DMSO-d₆) δ 7.94 (br s, 1H), 7.90 (d, J=7.9 Hz, 1H), 7.59 (d, J=10.1 Hz, 1H), 7.49-7.43 (m, 4H), 6.85 (br d, J=8.7 Hz, 2H), 6.24 (s, 2H), 5.04-4.96 (m, 1H), 3.87-3.80 (m, 2H), 3.59-3.53 (m, 2H), 3.43-3.39 (m, 2H), 2.97-2.93 (m, 2H); LCMS: [M+H]⁺=405.43.

### Example 147: 6-(2-amino-5-(4-((2,2-dimethylpiperidin-4-yl)oxy)phenyl)-6-fluoropyridin-3-yl)-3,4-dihydroisoquinolin-1(2H)-one (I-147)

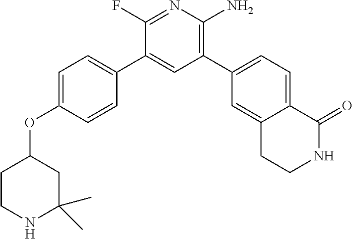 [SMILES: CC1(C)CC(Oc2ccc(-c3cc(-c4ccc5c(c4)CCNC5=O)c(N)nc3F)cc2)CCN1]

Step 1: tert-butyl 4-(4-bromophenoxy)-2,2-dimethylpiperidine-1-carboxylate

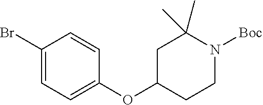 [SMILES: CC(C)(C)OC(=O)N1CCC(Oc2ccc(Br)cc2)CC1(C)C]

To a stirred solution of 4-bromophenol (264 mg, 1.526 mmol), tert-butyl 4-hydroxy-2,2-dimethylpiperidine-1-carboxylate (350 mg, 1.53 mmol) and triphenylphosphine (400 mg, 1.53 mmol) in anhydrous THF (10 mL) was added diisopropyl azodicarboxylate (0.301 mL, 1.53 mmol) slowly and at RT. The mixture was allowed to stir in a sealed flask at 50° C. for 3 d, and dry loaded onto Celite® and purified by flash chromatography (0-30%, Hexanes/EtOAc) to afford the product (317.6 mg, 54%) as a white powder which was used for the next step. LCMS: [M-tBu+H]⁺=328.10.

Step 2: tert-butyl 4-(4-(6-amino-2-fluoro-5-(1-oxo-1,2,3,4-tetrahydroisoquinolin-6-yl)pyridin-3-yl)phenoxy)-2,2-dimethylpiperidine-1-carboxylate

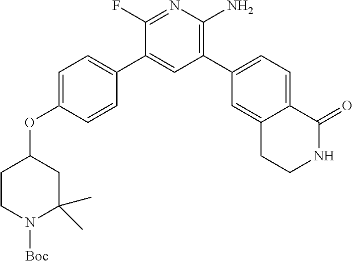 [SMILES: CC(C)(C)OC(=O)N1CCC(Oc2ccc(-c3cc(-c4ccc5c(c4)CCNC5=O)c(N)nc3F)cc2)CC1(C)C]

A procedure similar to Example 43, Step 5 using tert-butyl 4-(4-bromophenoxy)-2,2-dimethylpiperidine-1-carboxylate (130 mg, 0.339 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (130 mg, 0.339 mmol), 1,4-dioxane (3 mL), K₃PO₄ (216 mg, 1.02 mmol) in water (1 mL) and XPhos Pd G2 (26.7 mg, 0.034 mmol) afforded the product (53.7 mg, 28%) as a beige powder which was used for the next step. LCMS: [M+H]⁺=561.48.

Step 3: 6-(2-amino-5-(4-((2,2-dimethylpiperidin-4-yl)oxy)phenyl)-6-fluoropyridin-3-yl)-3,4-dihydroisoquinolin-1(2H)-one

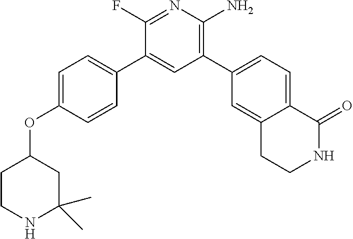 [SMILES: CC1(C)CC(Oc2ccc(-c3cc(-c4ccc5c(c4)CCNC5=O)c(N)nc3F)cc2)CCN1]

A procedure similar to Example 144, Step 3 using tert-butyl 4-(4-(6-amino-2-fluoro-5-(1-oxo-1,2,3,4-tetrahydroisoquinolin-6-yl)pyridin-3-yl)phenoxy)-2,2-dimethylpiperidine-1-carboxylate (53.7 mg, 0.096 mmol), TFA (2 mL, 26.1 mmol) in CH₂Cl₂ (4 mL) afforded the title compound (37.1 mg, 80%) as a beige powder. ¹H NMR (500 MHz, DMSO-d₆) δ 7.96-7.92 (m, 1H), 7.90 (d, J=7.9 Hz, 1H), 7.59 (d, J=10.1 Hz, 1H), 7.49-7.42 (m, 4H), 6.97 (d, J=8.8 Hz, 2H), 6.22 (s, 2H), 4.61-4.53 (m, 1H), 3.43-3.38 (m, 2H), 2.98-2.92 (m, 2H), 2.89-2.74 (m, 2H), 2.00-1.93 (m, 1H), 1.89-1.82 (m, 1H), 1.37-1.27 (m, 1H), 1.24 (br t, J=11.3 Hz, 1H), 1.10 (s, 3H), 1.08 (s, 3H); LCMS: [M+H]⁺=461.49.

### Example 148: 6-(2-amino-5-(2,3-difluoro-4-(piperidin-4-yloxy)phenyl)-6-fluoropyridin-3-yl)-3,4-dihydroisoquinolin-1(2H)-one (I-148)

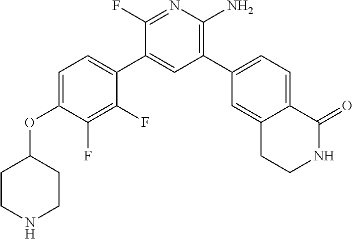 [SMILES: Nc1nc(F)c(-c2ccc(OC3CCNCC3)c(F)c2F)cc1-c1ccc2c(c1)CCNC2=O]

Step 1: tert-butyl 4-(4-bromo-2,3-difluorophenoxy)piperidine-1-carboxylate

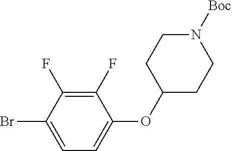 [SMILES: CC(C)(C)OC(=O)N1CCC(Oc2ccc(Br)c(F)c2F)CC1]

A procedure analogous to Example 147, Step 1 using 4-bromo-2,3-difluorophenol (523.8 mg, 2.51 mmol), tert-butyl 4-hydroxy-1-piperidinecarboxylate (504 mg, 2.51 mmol), triphenylphosphine (657 mg, 2.51 mmol), THF (10 mL), diisopropyl azodicarboxylate (0.493 mL, 2.51 mmol) afforded the product (730.9 mg, 46%) and 4-bromo-2,3-difluorophenol with a ratio of 62:38 and as a colorless oil which was used for the next step. LCMS: [M-Me+H]⁺=377.21.

Step 2: tert-butyl 4-(4-(6-amino-2-fluoro-5-(1-oxo-1,2,3,4-tetrahydroisoquinolin-6-yl)pyridin-3-yl)-2,3-difluorophenoxy)piperidine-1-carboxylate

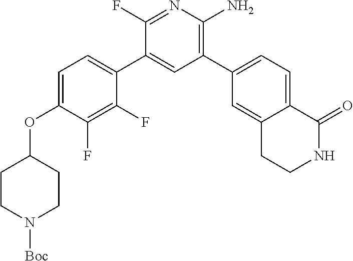 [SMILES: CC(C)(C)OC(=O)N1CCC(Oc2ccc(-c3cc(-c4ccc5c(c4)CCNC5=O)c(N)nc3F)c(F)c2F)CC1]

A procedure similar to Example 43, Step 5 using tert-butyl 4-(4-bromo-2,3-difluorophenoxy)piperidine-1-carboxylate (173 mg, 0.441 mmol), 6-(2-amino-5-bromo-6-fluoropyridin-3-yl)-3,4-dihydroisoquinolin-1(2H)-one (130 mg, 0.339 mmol), 1,4-dioxane (3 mL), K₃PO₄ (216 mg, 1.02 mmol) in water (1 mL) and XPhos Pd G2 (26.7 mg, 0.034 mmol) afforded the product (83.3 mg, 43%) as a white powder. LCMS: [M+H]⁺=569.45.

Step 3: 6-(2-amino-5-(2,3-difluoro-4-(piperidin-4-yloxy)phenyl)-6-fluoropyridin-3-yl)-3,4-dihydroisoquinolin-1(2H)-one

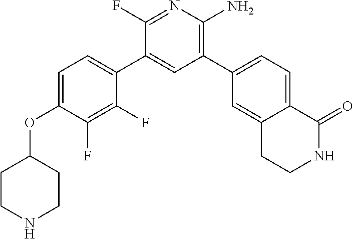 [SMILES: Nc1nc(F)c(-c2ccc(OC3CCNCC3)c(F)c2F)cc1-c1ccc2c(c1)CCNC2=O]

A procedure similar to Example 144, Step 3 using tert-butyl 4-(4-(6-amino-2-fluoro-5-(1-oxo-1,2,3,4-tetrahydroisoquinolin-6-yl)pyridin-3-yl)-2,3-difluorophenoxy)piperidine-1-carboxylate (83.3 mg, 0.147 mmol), TFA (2 mL, 26.1 mmol) and CH₂Cl₂ (4 mL) afforded the title compound (56.5 mg, 78%) as a beige powder. ¹H NMR (500 MHz, DMSO-d₆) δ 7.94 (br s, 1H), 7.90 (d, J=7.8 Hz, 1H), 7.53 (d, J=9.5 Hz, 1H), 7.47-7.41 (m, 2H), 7.20 (s, 1H), 7.14 (br d, J=7.8 Hz, 1H), 6.42 (s, 2H), 4.56-4.48 (m, 1H), 3.43-3.37 (m, 2H), 2.94 (s, 4H), 2.61-2.53 (m, 2H), 1.97-1.90 (m, 2H), 1.54-1.46 (m, 2H); LCMS: [M+H]⁺=469.46.

### Example 149: 6-(2-amino-5-(2,5-difluoro-4-(piperidin-4-yloxy)phenyl)-6-fluoropyridin-3-yl)-3,4-dihydroisoquinolin-1(2H)-one (I-149)

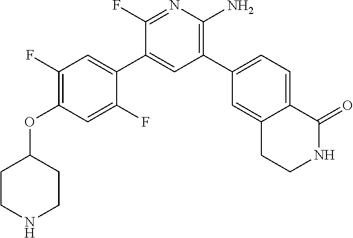 [SMILES: Nc1nc(F)c(-c2cc(F)c(OC3CCNCC3)cc2F)cc1-c1ccc2c(c1)CCNC2=O]

Step 1: tert-butyl 4-(4-bromo-2,5-difluorophenoxy)piperidine-1-carboxylate

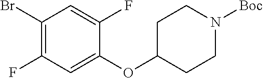 [SMILES: CC(C)(C)OC(=O)N1CCC(Oc2cc(F)c(Br)cc2F)CC1]

A procedure analogous to Example 147, Step 1 using 4-bromo-2,5-difluorophenol (556 mg, 2.66 mmol), tert-butyl 4-hydroxy-1-piperidinecarboxylate (535.7 mg, 2.66 mmol), triphenylphosphine (698 mg, 2.66 mmol), THF (10 mL) and diisopropyl azodicarboxylate (0.524 ml, 2.66 mmol) afforded the product (867.1 mg, 58%) and 4-bromo-2,5-difluorophenol with a ratio of 70:30 and as a colorless oil which was used for the next step. LCMS: [M-Me+H]⁺=377.21.

Step 2: tert-butyl 4-(4-(6-amino-2-fluoro-5-(1-oxo-1,2,3,4-tetrahydroisoquinolin-6-yl)pyridin-3-yl)-2,5-difluorophenoxy)piperidine-1-carboxylate

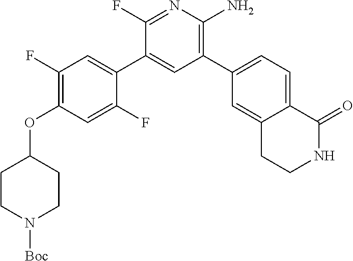 [SMILES: CC(C)(C)OC(=O)N1CCC(Oc2cc(F)c(-c3cc(-c4ccc5c(c4)CCNC5=O)c(N)nc3F)cc2F)CC1]

A procedure analogous to Example 43, Step 5 using tert-butyl 4-(4-bromo-2,5-difluorophenoxy)piperidine-1-carboxylate (174 mg, 0.444 mmol), 6-(2-amino-5-bromo-6-fluoropyridin-3-yl)-3,4-dihydroisoquinolin-1(2H)-one (131 mg, 0.342 mmol), 1,4-dioxane (3 ml), K₃PO₄ (218 mg, 1.03 mmol) in water (1 mL) and XPhos Pd G2 (26.9 mg, 0.034 mmol) afforded the product (85.3 mg, 44%) as a white powder. LCMS: [M+H]⁺=569.45.

Step 3: 6-(2-amino-5-(2,5-difluoro-4-(piperidin-4-yloxy)phenyl)-6-fluoropyridin-3-yl)-3,4-dihydroisoquinolin-1(2H)-one

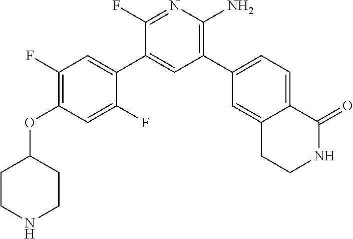 [SMILES: Nc1nc(F)c(-c2cc(F)c(OC3CCNCC3)cc2F)cc1-c1ccc2c(c1)CCNC2=O]

A procedure analogous to Example 144, Step 3 using tert-butyl 4-(4-(6-amino-2-fluoro-5-(1-oxo-1,2,3,4-tetrahydroisoquinolin-6-yl)pyridin-3-yl)-2,5-difluorophenoxy)piperidine-1-carboxylate (85.3 mg, 0.150 mmol), TFA (2 mL, 26.1 mmol) and CH₂Cl₂ (4 mL) afforded the title compound (60.3 mg, 82%) as a beige powder. ¹H NMR (500 MHz, DMSO-d₆) δ 7.94 (br s, 1H), 7.90 (d, J=7.9 Hz, 1H), 7.53 (d, J=9.7 Hz, 1H), 7.48-7.34 (m, 3H), 7.27 (dd, J=11.6, 7.2 Hz, 1H), 6.39 (s, 2H), 4.55-4.47 (m, 1H), 3.43-3.38 (m, 2H), 2.95 (br d, J=7.1 Hz, 4H), 2.61-2.54 (m, 2H), 1.97-1.89 (m, 2H), 1.53-1.42 (m, 2H); LCMS: [M+H]⁺=477.44.

### Example 150: 6-(2-amino-6-fluoro-5-(2-methoxy-4-(piperidin-4-yloxy)phenyl)pyridin-3-yl)-3,4-dihydroisoquinolin-1(2H)-one (I-150)

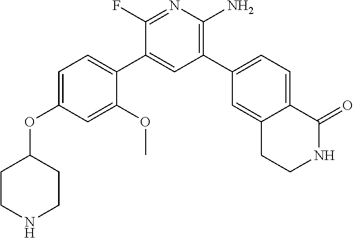 [SMILES: COc1cc(OC2CCNCC2)ccc1-c1cc(-c2ccc3c(c2)CCNC3=O)c(N)nc1F]

Step 1: tert-butyl 4-(4-(6-amino-2-fluoro-5-(1-oxo-1,2,3,4-tetrahydroisoquinolin-6-yl)pyridin-3-yl)-3-methoxyphenoxy)piperidine-1-carboxylate

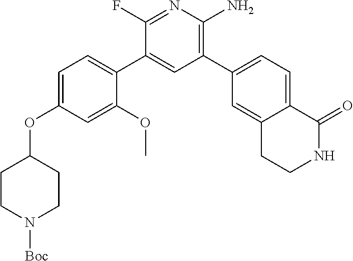 [SMILES: COc1cc(OC2CCN(C(=O)OC(C)(C)C)CC2)ccc1-c1cc(-c2ccc3c(c2)CCNC3=O)c(N)nc1F]

A procedure similar to Example 43, Step 5 using 6-(2-amino-5-bromo-6-fluoropyridin-3-yl)-3,4-dihydroisoquinolin-1(2H)-one (131 mg, 0.342 mmol), tert-butyl 4-(4-bromo-3-methoxyphenoxy)piperidine-1-carboxylate (158 mg, 0.410 mmol), 1,4-dioxane (3 mL) K₃PO₄ (218 mg, 1.03 mmol) in water (1 mL) and XPhos Pd G2 (26.9 mg, 0.034 mmol) afforded the product (73.0 mg, 38.0%) as a beige powder. LCMS: [M+H]⁺ 563.43.

Step 2: 6-(2-amino-6-fluoro-5-(2-methoxy-4-(piperidin-4-yloxy)phenyl)pyridin-3-yl)-3,4-dihydroisoquinolin-1(2H)-one

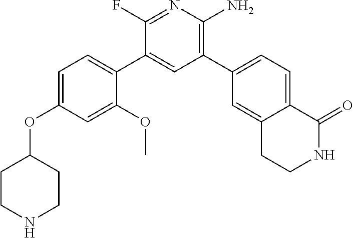 [SMILES: COc1cc(OC2CCNCC2)ccc1-c1cc(-c2ccc3c(c2)CCNC3=O)c(N)nc1F]

A procedure similar to Example 144, Step 3 using tert-butyl 4-(4-(6-amino-2-fluoro-5-(1-oxo-1,2,3,4-tetrahydroisoquinolin-6-yl)pyridin-3-yl)-3-methoxyphenoxy)piperidine-1-carboxylate (73.0 mg, 0.130 mmol), TFA (2 mL, 26.1 mmol) and CH₂Cl₂ (4 mL) afforded the title compound (53.0 mg, 84%) as a beige powder. ¹H NMR (500 MHz, DMSO-d₆) δ 7.92 (br s, 1H), 7.89 (d, J=7.9 Hz, 1H), 7.46-7.34 (m, 3H), 7.13 (d, J=8.2 Hz, 1H), 6.63-6.57 (m, 2H), 6.13 (s, 2H), 4.49-4.41 (m, 1H), 3.73 (s, 3H), 3.42-3.37 (m, 2H), 2.94 (br s, 4H), 2.63-2.56 (m, 2H), 1.97-1.89 (m, 2H), 1.51-1.42 (m, 2H); LCMS: [M+H]⁺=463.44.

### Example 151: 6-(2-amino-6-fluoro-5-(3-methoxy-4-(piperidin-4-yloxy)phenyl)pyridin-3-yl)-3,4-dihydroisoquinolin-1(2H)-one (I-151)

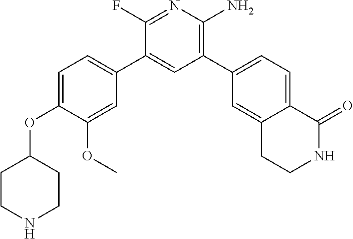 [SMILES: COc1cc(-c2cc(-c3ccc4c(c3)CCNC4=O)c(N)nc2F)ccc1OC1CCNCC1]

Step 1: tert-butyl 4-(4-(6-amino-2-fluoro-5-(1-oxo-1,2,3,4-tetrahydroisoquinolin-6-yl)pyridin-3-yl)-2-methoxyphenoxy)piperidine-1-carboxylate

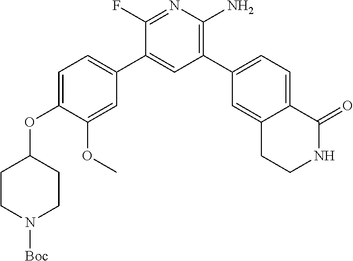 [SMILES: COc1cc(-c2cc(-c3ccc4c(c3)CCNC4=O)c(N)nc2F)ccc1OC1CCN(C(=O)OC(C)(C)C)CC1]

A procedure similar to Example 43, Step 5 using 4-(1-Boc-piperidin-4-yloxy)-3-methoxyphenylboronic acid, pinacol ester (79 mg, 0.183 mmol), 6-(2-amino-5-bromo-6-fluoropyridin-3-yl)-3,4-dihydroisoquinolin-1(2H)-one (55.8 mg, 0.166 mmol), 1,4-Dioxane (3 ml), Cs₂CO₃ (162 mg, 0.498 mmol) in water (1 mL) and Pd(dppf)Cl₂ (18.22 mg, 0.025 mmol) afforded the product (88.7 mg, 95%) as a light brown powder. LCMS: [M+H]⁺=563.51.

Step 2: 6-(2-amino-6-fluoro-5-(3-methoxy-4-(piperidin-4-yloxy)phenyl)pyridin-3-yl)-3,4-dihydroisoquinolin-1(2H)-one

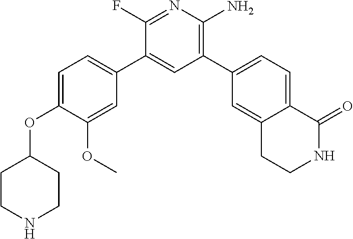 [SMILES: COc1cc(-c2cc(-c3ccc4c(c3)CCNC4=O)c(N)nc2F)ccc1OC1CCNCC1]

A procedure similar to Example 144, Step 3 using tert-butyl 4-(4-(6-amino-2-fluoro-5-(1-oxo-1,2,3,4-tetrahydroisoquinolin-6-yl)pyridin-3-yl)-2-methoxyphenoxy)piperidine-1-carboxylate (88.7 mg, 0.158 mmol), TFA (2 mL, 26.1 mmol) and CH₂Cl₂ (4 mL) afforded the title compound (63.6 mg, 83%) as a beige powder. ¹H NMR (500 MHz, DMSO-d₆) δ 7.94 (br s, 1H), 7.90 (d, J=7.9 Hz, 1H), 7.64 (d, J=10.1 Hz, 1H), 7.48-7.42 (m, 2H), 7.10 (s, 1H), 7.02 (s, 2H), 6.23 (s, 2H), 4.35-4.28 (m, 1H), 3.79 (s, 3H), 3.43-3.37 (m, 2H), 2.95 (br s, 4H), 2.59-2.52 (m, 2H), 1.92-1.85 (m, 2H), 1.51-1.41 (m, 2H); LCMS: [M+H]⁺=463.44.

### Example 152: 6-(2-amino-6-fluoro-5-(4-(1-(oxetan-3-yl)piperidin-4-yl)phenyl)pyridin-3-yl)-3,4-dihydroisoquinolin-1(2H)-one (I-152)

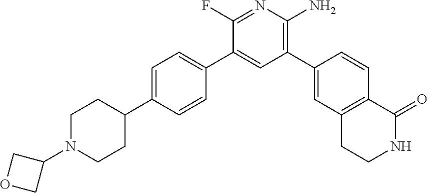 [SMILES: Nc1nc(F)c(-c2ccc(C3CCN(C4COC4)CC3)cc2)cc1-c1ccc2c(c1)CCNC2=O]

To a mixture of 6-(2-amino-6-fluoro-5-(4-(piperidin-4-yl)phenyl)pyridin-3-yl)-3,4-dihydroisoquinolin-1(2H)-one (27 mg, 0.065 mmol), oxetan-3-one (3.99 μl, 0.068 mmol) and AcOH (0.015 mL, 0.259 mmol) in DCE (4 mL), was added NaBH(OAc)₃ (41.2 mg, 0.194 mmol) and the slurry obtained was stirred overnight at RT. The reaction mixture was quenched with saturated aqueous NaHCO₃ solution. The layers were separated and the aqueous layer was further extracted with DCM (x3). The combined extract was dried and concentrated onto Celite®. Silica gel chromatography (eluting with DCM containing 0-4% MeOH and 0-0.4% NH40H), followed by reverse phase column (C18, 0-30% acetonitrile/water), and then passing through a Waters PoraPak CX cartridge afforded the title compound as an off white powder (8.5 mg, 25% based on purity of 90%). ¹H NMR (500 MHz, DMSO-d₆) δ 7.98-7.93 (m, 1H), 7.92-7.88 (m, 1H), 7.66-7.61 (m, 1H), 7.51-7.45 (m, 4H), 7.30 (d, J=8.2 Hz, 2H), 6.38-6.20 (m, 2H), 4.58-4.53 (m, 2H), 4.48-4.43 (m, 2H), 3.44-3.39 (m, 5H), 2.99-2.92 (m, 2H), 2.86-2.77 (m, 2H), 2.58-2.53 (m, 1H), 1.91-1.85 (m, 2H), 1.80-1.75 (m, 2H), 1.73-1.66 (m, 2H); LCMS: [M+H]⁺=473.62.

### Example 153: 6-(2-amino-6-fluoro-5-(4-(1-(oxetan-3-yl)piperidin-2-yl)phenyl)pyridin-3-yl)-3,4-dihydroisoquinolin-1(2H)-one (I-153)

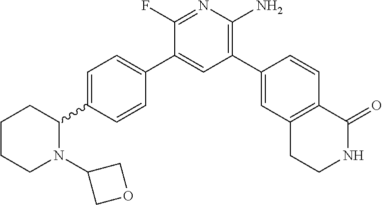 [SMILES: Nc1nc(F)c(-c2ccc(C3CCCCN3C3COC3)cc2)cc1-c1ccc2c(c1)CCNC2=O]

A procedure analogous to Example 152 using 6-(2-amino-6-fluoro-5-(4-(piperidin-2-yl)phenyl)pyridin-3-yl)-3,4-dihydroisoquinolin-1(2H)-one (30 mg, 0.058 mmol) with oxetan-3-one (3.54 μl, 0.061 mmol). Silica gel chromatography (eluting with DCM containing 0-3% MeOH and 0-0.3% NH₄OH), followed by reverse phase column (C18, 0-30% acetonitrile/water afforded the title compound (4 mg, 14%) as a white powder. ¹H NMR (500 MHz, DMSO-d₆) δ 7.98-7.93 (m, 1H), 7.93-7.89 (m, 1H), 7.68-7.63 (m, 1H), 7.52-7.46 (m, 4H), 7.39-7.33 (m, 2H), 6.38-6.26 (m, 2H), 4.53-4.45 (m, 1H), 4.33-4.26 (m, 1H), 3.80-3.73 (m, 1H), 3.57-3.51 (m, 1H), 3.45-3.39 (m, 4H), 2.99-2.93 (m, 2H), 2.93-2.88 (m, 1H), 2.84-2.78 (m, 1H), 1.96-1.87 (m, 1H), 1.81-1.74 (m, 1H), 1.72-1.64 (m, 2H), 1.63-1.54 (m, 2H); LCMS: [M+H]⁺=473.62.

### Example 154: 6-(2-amino-6-fluoro-5-(4-(4-(oxetan-3-yl)morpholin-3-yl)phenyl)pyridin-3-yl)-3,4-dihydroisoquinolin-1(2H)-one (I-154)

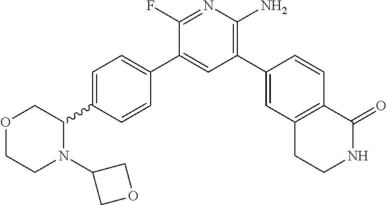 [SMILES: Nc1nc(F)c(-c2ccc(C3COCCN3C3COC3)cc2)cc1-c1ccc2c(c1)CCNC2=O]

A procedure analogous to Example 152 using 6-(2-amino-6-fluoro-5-(4-(morpholin-3-yl)phenyl)pyridin-3-yl)-3,4-dihydroisoquinolin-1(2H)-one (37 mg, 0.088 mmol) with oxetan-3-one (5.44 μl, 0.093 mmol)) afforded the title compound (6.5 mg, 14%) as a white powder. ¹H NMR (500 MHz, DMSO-d₆) δ 8.00-7.93 (m, 1H), 7.93-7.86 (m, 1H), 7.66 (br d, J=10.0 Hz, 1H), 7.58-7.50 (m, 2H), 7.50-7.47 (m, 1H), 7.47-7.44 (m, 1H), 7.42-7.35 (m, 2H), 6.47-6.27 (m, 2H), 4.64-4.54 (m, 1H), 4.38-4.29 (m, 1H), 3.94-3.84 (m, 2H), 3.70-3.57 (m, 4H), 3.53-3.48 (m, 1H), 3.41 (br s, 2H), 3.24-3.17 (m, 1H), 2.96 (br t, J=6.1 Hz, 2H), 2.81-2.74 (m, 1H), 2.30-2.18 (m, 1H); LCMS: [M+H]⁺=475.56.

### Example 155: 6-(2-amino-6-fluoro-5-(4-(1-(oxetan-3-yl)-1,2,3,6-tetrahydropyridin-4-yl)phenyl)pyridin-3-yl)-3,4-dihydroisoquinolin-1(2H)-one (I-155)

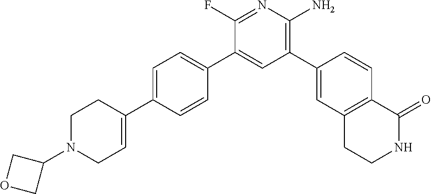 [SMILES: Nc1nc(F)c(-c2ccc(C3=CCN(C4COC4)CC3)cc2)cc1-c1ccc2c(c1)CCNC2=O]

A procedure analogous to Example 152, by the reductive amination of 6-(2-amino-6-fluoro-5-(4-(1,2,3,6-tetrahydropyridin-4-yl)phenyl)pyridin-3-yl)-3,4-dihydroisoquinolin-1(2H)-one TFA (35 mg, 0.066 mmol) with oxetan-3-one (4.07 μL, 0.070 mmol), afforded the title compound (9.5 mg, 30%) as a yellow powder. ¹H NMR (500 MHz, DMSO-d₆) δ 7.99-7.93 (m, 1H), 7.93-7.88 (m, 1H), 7.72-7.64 (m, 1H), 7.57-7.51 (m, 2H), 7.51-7.40 (m, 4H), 6.41-6.27 (m, 2H), 6.26-6.17 (m, 1H), 4.64-4.56 (m, 2H), 4.56-4.47 (m, 2H), 3.61-3.53 (m, 1H), 3.45-3.40 (m, 2H), 3.37-3.34 (m, 4H), 3.04-2.99 (m, 2H), 2.98-2.93 (m, 2H); LCMS: [M+H]⁺=471.55.

### Example 156: 6-(2-amino-6-fluoro-5-(4-((1R,5S)-3-(oxetan-3-yl)-3-azabicyclo[3.1.0]hexan-1-yl)phenyl)pyridin-3-yl)-3,4-dihydroisoquinolin-1(2H)-one (I-156)

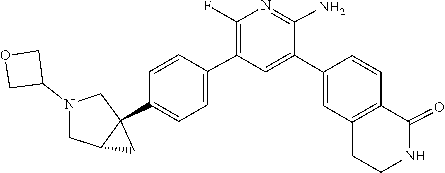 [SMILES: Nc1nc(F)c(-c2ccc([C@@]34C[C@@H]3CN(C3COC3)C4)cc2)cc1-c1ccc2c(c1)CCNC2=O]

Step 1: (1R,5S)-1-(4-bromophenyl)-3-(oxetan-3-yl)-3-azabicyclo[3.1.0]hexane

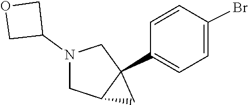 [SMILES: Brc1ccc([C@@]23C[C@@H]2CN(C2COC2)C3)cc1]

A stirred anhydrous CH₂Cl₂ (15 mL) solution of (1R,5S)-1-(4-bromophenyl)-3-azabicyclo[3.1.0]hexane (220 mg, 0.924 mmol) was treated with glacial acetic acid (0.11 mL, 1.8 mmol) and oxetan-3-one (0.11 mL, 1.8 mmol) at RT. After 1.25 h of stirring at RT, NaBH(OAc)₃ (490 mg, 2.31 mmol) was added in one portion and stirring was continued at RT for 2 d. Later, MeOH (2 mL) and satd aq NaHCO₃ (6 mL) were added. The separated aqueous layer was extracted with CH₂Cl₂ and combined organics were filtered through a plug of Na₂SO₄ and concentrated under reduced pressure. The dried in vacuo product (an orange gum, 267 mg, quant) was used without further purification in the following step. LCMS: [M+H]⁺=294.21/296.24.

Step 2: 6-(2-amino-6-fluoro-5-(4-((1R,5S)-3-(oxetan-3-yl)-3-azabicyclo[3.1.0]hexan-1-yl)phenyl)pyridin-3-yl)-3,4-dihydroisoquinolin-1(2H)-one

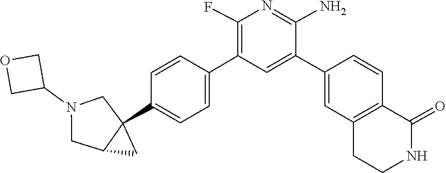 [SMILES: Nc1nc(F)c(-c2ccc([C@@]34C[C@@H]3CN(C3COC3)C4)cc2)cc1-c1ccc2c(c1)CCNC2=O]

A procedure similar to Example 1, General Method A using (1R,5S)-1-(4-bromophenyl)-3-(oxetan-3-yl)-3-azabicyclo[3.1.0]hexane (70 mg, 0.24 mmol), 6-(2-amino-6-fluoro-5-(4,4,5,5-tetramethyl-1,3,2-dioxaborolan-2-yl)pyridin-3-yl)-3,4-dihydroisoquinolin-1(2H)-one (96 mg, 0.25 mmol), PdCl₂dppf (17 mg, 0.024 mmol), Cs₂CO₃ (233 mg, 0.714 mmol), H₂O (2.0 mL) and DME (4 mL) afforded the title compound as a white solid (5.0 mg, 5%). ¹H NMR (500 MHz, CDCl₃) δ 8.11 (d, J=8.0 Hz, 1H), 7.51 (d, J=9.5 Hz, 1H), 7.31-7.42 (m, 3H), 7.26 (s, 1H), 7.12 (d, J=8.3 Hz, 2H), 6.03 (br s, 1H), 4.67 (s, 2H), 4.54-4.65 (m, 4H), 3.76 (quin, J=6.4 Hz, 1H), 3.55 (td, J=6.5, 2.8 Hz, 2H), 3.25 (d, J=8.2 Hz, 1H), 2.95-3.04 (m, 3H), 2.59 (d, J=8.2 Hz, 1H), 2.48 (dd, J=8.3, 3.6 Hz, 1H), 1.71 (dt, J=7.9, 4.0 Hz, 1H), 1.45 (t, J=4.3 Hz, 1H), 0.73-0.86 (m, 1H); LCMS: [M+H]⁺=471.49.

### Example 157: 6-(2-amino-5-(4-(3-(dimethylamino)pyrrolidin-1-yl)phenyl)-6-fluoropyridin-3-yl)-3,4-dihydroisoquinolin-1(2H)-one (I-157)

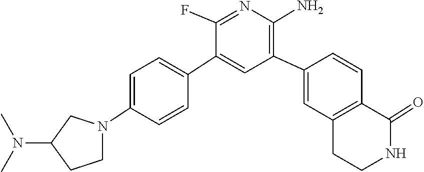 [SMILES: CN(C)C1CCN(c2ccc(-c3cc(-c4ccc5c(c4)CCNC5=O)c(N)nc3F)cc2)C1]

Step 1: 1-(4-chloro-3-fluorophenyl)-N,N-dimethylpyrrolidin-3-amine

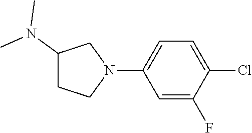 [SMILES: CN(C)C1CCN(c2ccc(Cl)c(F)c2)C1]

A procedure analogous to Example 108, Step 1 using 3-(dimethylamino)pyrrolidine (0.234 mL, 1.43 mmol), 4-bromo-1-chloro-2-fluorobenzene (0.139 mL, 1.19 mmol), tris(dibenzylideneacetone)dipalladium (0) (0.033 g, 0.036 mmol), 4,5-bis(diphenylphosphino)-9,9-dimethylxanthene (0.062 g, 0.107 mmol) and Cs₂CO₃ (1.17 g, 3.58 mmol) afforded the product (286 mg, 99%). LCMS: [M+H]⁺=243.19

Step 2: 1-(3-fluoro-4-(4,4,5,5-tetramethyl-1,3,2-dioxaborolan-2-yl)phenyl)-N,N-dimethylpyrrolidin-3-amine

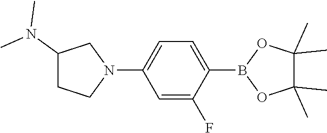

A procedure analogous to that described in Example 108, Step 2 using 1-(4-chloro-3-fluorophenyl)-N,N-dimethylpyrrolidin-3-amine (0.286 g, 1.18 mmol), bis(pinacolato)diboron (0.374 g, 1.47 mmol), KOAc (0.231 g, 2.36 mmol) and XPhos Pd G2 (0.070 g, 0.088 mmol) afforded the product (234 mg, 42%) that was judged to be approximately 70% pure and was used without further purification in the next step. LCMS: [M +H]⁺=335.24.

Step 3: 6-(2-amino-5-(4-(3-(dimethylamino)pyrrolidin-1-yl)phenyl)-6-fluoropyridin-3-yl)-3,4-dihydroisoquinolin-1(2H)-one

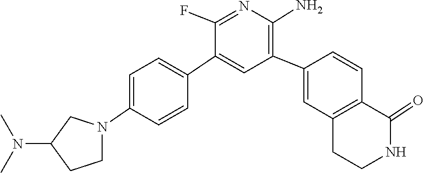 [SMILES: CN(C)C1CCN(c2ccc(-c3cc(-c4ccc5c(c4)CCNC5=O)c(N)nc3F)cc2)C1]

A procedure similar to Example 43, Step 5 using 6-(2-amino-6-fluoro-5-(4,4,5,5-tetramethyl-1,3,2-dioxaborolan-2-yl)pyridin-3-yl)-3,4-dihydroisoquinolin-1(2H)-one (0.075 g, 0.196 mmol), 1-(4-chlorophenyl)-N,N-dimethylpyrrolidin-3-amine (0.046 g, 0.205 mmol), XPhos Pd G2 (0.015 g, 0.020 mmol) and aqueous K₃PO₄ (0.376 mL of a 1.3 M solution, 0.489 mmol) afforded the title compound (31 mg, 36%) as a light grey powder. ¹H NMR (500 MHz, DMSO-d₆) δ 7.9-8.0 (m, 2H), 7.56 (d, J=10.1 Hz, 1H), 7.4-7.5 (m, 2H), 7.36 (d, J=7.8 Hz, 2H,), 6.59 (d, J=8.8 Hz, 2H), 6.11 (s, 2H), 3.46 (dd, J=8.9, 7.5 Hz, 1H), 3.4-3.4 (m, 3H), 3.31 (s, 2H), 3.25 (dt, J=9.5, 7.0 Hz, 1H), 3.05 (t, J=8.6 Hz, 1H), 2.96 (t, J=6.5 Hz, 2H), 2.8-2.8 (m, 1H), 2.21 (s, 6H), 2.1-2.2 (m, 1H), 1.8-1.9 (m, 1H); LCMS: [M+H]⁺=446.4.

### Example 158: 6-(2-amino-5-(4-(3-(ethyl(methyl)amino)pyrrolidin-1-yl)phenyl)-6-fluoropyridin-3-yl)-3,4-dihydroisoquinolin-1(2H)-one (I-158)

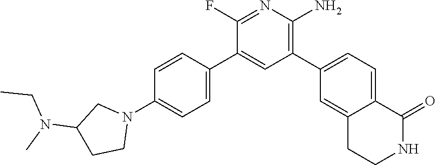 [SMILES: CCN(C)C1CCN(c2ccc(-c3cc(-c4ccc5c(c4)CCNC5=O)c(N)nc3F)cc2)C1]

Step 1: tert-butyl 3-(N-methylacetamido)pyrrolidine-1-carboxylate

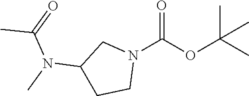 [SMILES: CC(=O)N(C)C1CCN(C(=O)OC(C)(C)C)C1]

Acetyl chloride (0.11 mL, 1.5 mmol) was added dropwise to a vial containing a solution of 1-boc-3-methylaminopyrrolidine (0.25 g, 1.2 mmol) and triethylamine (0.21 mL, 1.5 mmol) in DCM (5 mL) at RT. The reaction was allowed to stir at RT for 18 h. The reaction mixture was concentrated onto Celite®. Purification by flash chromatography (0.5-9.5% DCM/MeOH+0.5% NH₄OH) afforded the product (0.32 g, quantitative yield) as a clear oil. LCMS: [M+H]⁺=243.3.

Step 2: tert-butyl 3-(ethyl(methyl)amino)pyrrolidine-1-carboxylate

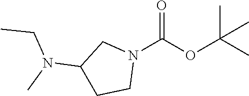 [SMILES: CCN(C)C1CCN(C(=O)OC(C)(C)C)C1]

A 2 M solution of borane dimethyl sulfide complex in THF (2.0 mL, 4.0 mmol) was added dropwise to a solution of tert-butyl 3-(N-methylacetamido)pyrrolidine-1-carboxylate (0.32 g, 1.3 mmol) in THF (6 mL) at 0° C. The ice bath was removed and the reaction mixture was heated to 60° C. for 4 h. After cooling to RT the reaction was carefully quenched with the dropwise addition of saturated NaHCO₃ (Aq.). Isolation of the desired product was achieved by a catch and release procedure using Biotage SCX2 silica gel to afford the product (0.20 g, 67%) as a clear oil. LCMS: [M+H]⁺ 229.3.

Step 3: N-ethyl-N-methylpyrrolidin-3-amine

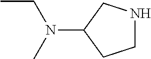

TFA (1.7 mL, 22 mmol) was added to a stirring solution of tert-butyl 3-(ethyl(methyl)amino)pyrrolidine-1-carboxylate (0.20 g, 0.89 mmol) in DCM (2 mL) at RT. The reaction was allowed to stir at RT for 18 h. The volatiles were removed under a stream of air and the isolation of the desired product was achieved by a catch and release procedure using Biotage SCX2 silica gel to afford the product (0.12 g, quantitative yield). LCMS: [M+H]⁺=129.1.

Step 4: 1-(4-chlorophenyl)-N-ethyl-N-methylpyrrolidin-3-amine

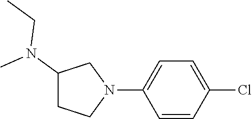 [SMILES: CCN(C)C1CCN(c2ccc(Cl)cc2)C1]

A 30 mL vial was charged with N-ethyl-N-methylpyrrolidin-3-amine (0.12 g, 0.94 mmol), 1-bromo-4-chlorobenzene (0.22 g, 1.1 mmol), tris(dibenzylideneacetone)dipalladium (0) (0.026 g, 0.028 mmol), 4,5-bis(diphenylphosphino)-9,9-dimethylxanthene (0.049 g, 0.084 mmol) and cesium carbonate (0.92 g, 2.8 mmol). The vial was sealed with a cap and septum and then the reaction vessel was evacuated and backfilled with nitrogen. Toluene (6 mL) was added and the reaction vessel was evacuated and backfilled with nitrogen an additional time. The reaction was heated in an aluminum block at 100° C. for >48 h. The reaction mixture was concentrated onto Celite® and purified by flash chromatography (0.5-10% DCM/MeOH+0.5% NH₄OH) to afford the product (0.13 g, 56%). LCMS: [M+H]⁺=239.0.

Step 5: 6-(2-amino-5-(4-(3-(ethyl(methyl)amino)pyrrolidin-1-yl)phenyl)-6-fluoropyridin-3-yl)-3,4-dihydroisoquinolin-1(2H)-one

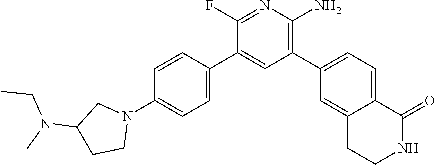 [SMILES: CCN(C)C1CCN(c2ccc(-c3cc(-c4ccc5c(c4)CCNC5=O)c(N)nc3F)cc2)C1]

A 30 mL vial was charged with 6-(2-amino-6-fluoro-5-(4,4,5,5-tetramethyl-1,3,2-dioxaborolan-2-yl)pyridin-3-yl)-3,4-dihydroisoquinolin-1(2H)-one (0.075 g, 0.20 mmol), 1-(4-chlorophenyl)-N-ethyl-N-methylpyrrolidin-3-amine (0.049 g, 0.21 mmol) and XPhos Pd G2 (0.015 g, 0.020 mmol). The vial was sealed with a cap and septum and the reaction vessel was evacuated and backfilled with nitrogen. 1,4-dioxane (2 mL) and 1.3 M aqueous K₃PO₄ (0.38 mL, 0.49 mmol) were added and the reaction vessel was evacuated and backfilled with nitrogen an additional time. The reaction mixture was heated at 95° C. for 18 h. The reaction mixture was cooled to RT, concentrated onto Celite® and purified by flash chromatography (0.5-9.5% MeOH/DCM+0.5% NH₄OH). The product containing fractions were concentrated and further purified by reverse phase chromatography (Biotage SNAP C18; 5-50% MeCN/water+0.1% Formic Acid). Isolation of the title compound was achieved by a catch and release procedure using Biotage SCX2 silica gel to afford the product (24 mg, 27%). ¹H NMR (500 MHz, DMSO-d₆) δ 7.9-8.0 (m, 2H), 7.56 (d, J=10.3 Hz, 1H), 7.4-7.5 (m, 2H), 7.36 (d, J=7.7 Hz, 2H), 6.59 (d, J=8.8 Hz, 2H), 6.11 (s, 2H), 3.47 (br d, J=1.8 Hz, 1H), 3.4-3.4 (m, 4H), 3.25 (dt, J=9.5, 2.4 Hz, 1H), 3.0-3.1 (m, 2H), 2.96 (t, J=6.5 Hz, 2H), 2.19 (s, 3H), 2.1-2.2 (m, 1H), 1.8-1.9 (m, 1H), 1.01 (t, J=7.2 Hz, 3H); LCMS: [M+H]⁺=460.5.

### Example 159: 6-(2-amino-6-fluoro-5-(4-(3-(methyl(2,2,2-trifluoroethyl)amino)pyrrolidin-1-yl)phenyl)pyridin-3-yl)-3,4-dihydroisoquinolin-1(2H)-one (I-159)

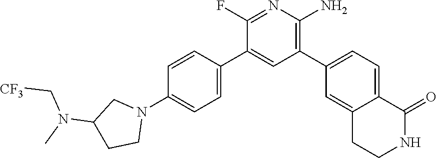 [SMILES: CN(CC(F)(F)F)C1CCN(c2ccc(-c3cc(-c4ccc5c(c4)CCNC5=O)c(N)nc3F)cc2)C1]

Step 1: tert-butyl 3-(2,2,2-trifluoro-N-methylacetamido)pyrrolidine-1-carboxylate

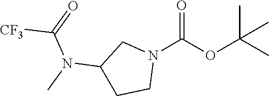 [SMILES: CN(C(=O)C(F)(F)F)C1CCN(C(=O)OC(C)(C)C)C1]

TFA (0.14 mL, 1.9 mmol) was activated with HATU (0.71 g, 1.9 mmol) and N,N-diisopropylethylamine (0.33 mL, 1.9 mmol) in DMF (1 mL) at RT. After stirring for 10 minutes the solution of activated acid was transferred by pipette to a vial containing a solution of 1-boc-3-methylaminopyrrolidine (0.25 g, 1.3 mmol) in DMF (1 mL) at RT. The reaction was allowed to stir at RT for 18 h and the reaction mixture was then concentrated onto Celite®. Purification by flash chromatography (25-75% EtOAC/hexanes) to afford the product (0.24 g, 64%) as a clear oil. LCMS: [M+H]⁺=297.3.

Step 2: tert-butyl 3-(methyl(2,2,2-trifluoroethyl)amino)pyrrolidine-1-carboxylate

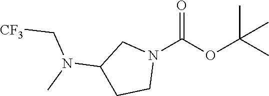 [SMILES: CN(CC(F)(F)F)C1CCN(C(=O)OC(C)(C)C)C1]

A 2M solution of borane dimethyl sulfide complex in THF (1.2 mL, 2.4 mmol) was added dropwise to a solution of tert-butyl 3-(2,2,2-trifluoro-N-methylacetamido)pyrrolidine-1-carboxylate (0.24 g, 0.80 mmol) in THF (6 mL) at 0° C. The ice bath was removed and the reaction mixture was heated to 60° C. for 4 h. After cooling to RT the reaction was carefully quenched with the dropwise addition of saturated NaHCO₃ (Aq.). Concentrating to dryness afforded the product (0.21 g, 94%) that was of sufficient purity to carry forward to the next step. LCMS: [M+H]⁺=282.2.

Step 3: N-methyl-N-(2,2,2-trifluoroethyl)pyrrolidin-3-amine

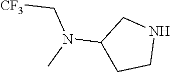

TFA (1.4 mL, 19 mmol) was added to a stirring solution of tert-butyl 3-(methyl(2,2,2-trifluoroethyl)amino)pyrrolidine-1-carboxylate (0.21 g, 0.75 mmol) in DCM (2 mL) at RT. The reaction was allowed to stir at RT for 18 h. The volatiles were removed under a stream of air and the isolation of the desired product was achieved by a catch and release procedure using Biotage SCX2 silica gel to afford the product (0.14 g, quantitative yield). LCMS: [M+H]⁺ 183.1.

Step 4: 1-(4-chlorophenyl)-N-methyl-N-(2,2,2-trifluoroethyl)pyrrolidin-3-amine

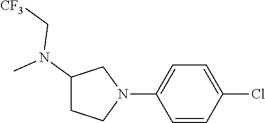 [SMILES: CN(CC(F)(F)F)C1CCN(c2ccc(Cl)cc2)C1]

A procedure analogous to Example 108, Step 1 using N-methyl-N-(2,2,2-trifluoroethyl)pyrrolidin-3-amine (0.14 g, 0.77 mmol), 1-bromo-4-chlorobenzene (0.18 g, 0.92 mmol), tris(dibenzylideneacetone)dipalladium (0) (0.021 g, 0.023 mmol), 4,5-bis(diphenylphosphino)-9,9-dimethylxanthene (0.040 g, 0.069 mmol) and Cs₂CO₃ (0.75 g, 2.3 mmol) afforded the product (0.13 g, 56%). LCMS: [M+H]⁺=293.1.

Step 5: 6-(2-amino-6-fluoro-5-(4-(3-(methyl(2,2,2-trifluoroethyl)amino)pyrrolidin-1-yl)phenyl)pyridin-3-yl)-3,4-dihydroisoquinolin-1(2H)-one

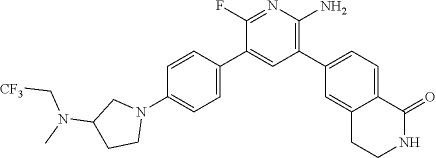 [SMILES: CN(CC(F)(F)F)C1CCN(c2ccc(-c3cc(-c4ccc5c(c4)CCNC5=O)c(N)nc3F)cc2)C1]

A procedure similar to Example 43, Step 5 using 6-(2-amino-6-fluoro-5-(4,4,5,5-tetramethyl-1,3,2-dioxaborolan-2-yl)pyridin-3-yl)-3,4-dihydroisoquinolin-1(2H)-one (0.075 g, 0.20 mmol), 1-(4-chlorophenyl)-N-methyl-N-(2,2,2-trifluoroethyl)pyrrolidin-3-amine (0.060 g, 0.21 mmol) and XPhos Pd G2 (0.015 g, 0.020 mmol), 1,4-dioxane (2 mL), 1.3 M aqueous K₃PO₄ (0.38 mL, 0.49 mmol) afforded the title compound (9 mg, 9%). ¹H NMR (500 MHz, DMSO-d₆) δ 7.9-8.0 (m, 2H), 7.56 (d, J=10.3 Hz, 1H), 7.4-7.5 (m, 2H), 7.37 (br d, J=8.1 Hz, 2H), 6.60 (d, J=8.7 Hz, 2H), 6.11 (s, 2H), 3.5-3.5 (m, 1H), 3.4-3.4 (m, 5H), 3.2-3.3 (m, 3H), 3.1-3.1 (m, 1H), 2.96 (br t, J=6.5 Hz, 2H), 2.44 (s, 3H), 2.2-2.2 (m, 1H), 1.8-1.9 (m, 1H); LCMS: [M+H]⁺=514.5.

### Example 160: (R)-6-(2-amino-6-fluoro-5-(4-(3-((2-methoxyethyl)(methyl)amino)pyrrolidin-1-yl)phenyl)pyridin-3-yl)-3,4-dihydroisoquinolin-1(2H)-one (I-160)

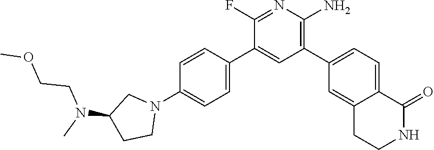 [SMILES: COCCN(C)[C@@H]1CCN(c2ccc(-c3cc(-c4ccc5c(c4)CCNC5=O)c(N)nc3F)cc2)C1]

Step 1: tert-butyl (R)-3-((2-methoxyethyl)(methyl)amino)pyrrolidine-1-carboxylate

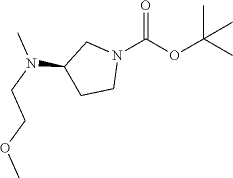 [SMILES: COCCN(C)[C@@H]1CCN(C(=O)OC(C)(C)C)C1]

A mixture of (S)-1-Boc-3-methanesulfonyloxy-pyrrolidine (0.28 g, 1.1 mmol), N-(2-methoxyethyl)methylamine (0.29 mL, 2.6 mmol) and N,N-diisopropylethylamine (0.50 mL, 2.6 mmol) was heated to 70° C. in a sealed tube for 18 h and then to 85° C. for >48 h. The reaction mixture was diluted with DCM and concentrated onto Celite®. Purification by flash chromatography [0.5-7.5% MeOH/DCM+0.5% NH₄OH] to afford the product (0.23 g, 83%) as a clear oil. LCMS: [M+H]⁺=259.4.

Step 2: (R)—N-(2-methoxyethyl)-N-methylpyrrolidin-3-amine

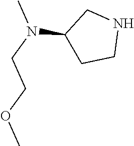

TFA (1.7 mL, 22 mmol) was added to a stirring solution of tert-butyl (R)-3-((2-methoxyethyl)(methyl)amino)pyrrolidine-1-carboxylate (0.23 g, 0.88 mmol) in DCM (2 mL) at RT. The reaction was allowed to stir at RT for 18 h. The volatiles were removed under a stream of air and the isolation of the desired product was achieved by a catch and release procedure using Biotage SCX2 silica gel to afford the product (0.14 g, quantitative yield). LCMS: [M+H]⁺=159.3.

Step 3: (R)-1-(4-chlorophenyl)-N-(2-methoxyethyl)-N-methylpyrrolidin-3-amine

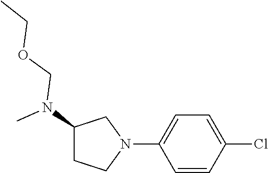 [SMILES: CCOCN(C)[C@@H]1CCN(c2ccc(Cl)cc2)C1]

A procedure analogous to Example 108, Step 1 using (R)—N-(2-methoxyethyl)-N-methylpyrrolidin-3-amine (0.14 g, 0.89 mmol), 1-bromo-4-chlorobenzene (0.20 g, 1.1 mmol), tris(dibenzylideneacetone)dipalladium (0) (0.024 g, 0.027 mmol), 4,5-bis(diphenylphosphino)-9,9-dimethylxanthene (0.046 g, 0.080 mmol) and Cs₂CO₃ (0.87 g, 2.7 mmol) afforded the product (0.14 g, 61%). LCMS: [M+H]⁺=269.3.

Step 4: (R)-6-(2-amino-6-fluoro-5-(4-(3-((2-methoxyethyl)(methyl)amino)pyrrolidin-1-yl)phenyl)pyridin-3-yl)-3,4-dihydroisoquinolin-1(2H)-one

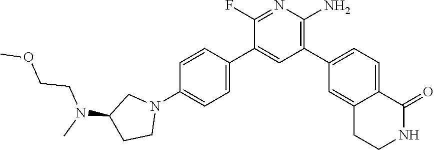 [SMILES: COCCN(C)[C@@H]1CCN(c2ccc(-c3cc(-c4ccc5c(c4)CCNC5=O)c(N)nc3F)cc2)C1]

A procedure similar to Example 43, Step 5 using 6-(2-amino-6-fluoro-5-(4,4,5,5-tetramethyl-1,3,2-dioxaborolan-2-yl)pyridin-3-yl)-3,4-dihydroisoquinolin-1(2H)-one (0.075 g, 0.20 mmol), (R)-1-(4-chlorophenyl)-N-(2-methoxyethyl)-N-methylpyrrolidin-3-amine (0.055 g, 0.21 mmol), XPhos Pd G2 (0.015 g, 0.020 mmol), 1,4-dioxane (2 mL) and 1.3 M aqueous K₃PO₄ (0.38 mL, 0.49 mmol) afforded the title compound (0.037 g, 39%). ¹H NMR (500 MHz, DMSO-d₆) δ 7.9-8.0 (m, 2H), 7.56 (d, J=10.3 Hz, 1H), 7.4-7.5 (m, 2H), 7.36 (d, J=7.9 Hz, 2H), 6.59 (d, J=8.7 Hz, 2H), 6.11 (s, 2H), 3.4-3.5 (m, 7H), 3.25 (s, 4H), 3.1-3.2 (m, 1H), 3.0-3.1 (m, 1H), 2.96 (br t, J=6.5 Hz, 2H), 2.60 (q, J=6.1 Hz, 2H), 2.25 (s, 3H), 2.17 (ddd, J=11.7, 6.8, 4.5 Hz, 1H), 1.8-1.9 (m, 1H); LCMS: [M+H]⁺=490.5.

### Example 161: (R)-6-(2-amino-5-(4-(3-((cyclopropylmethyl)(methyl)amino)pyrrolidin-1-yl)phenyl)-6-fluoropyridin-3-yl)-3,4-dihydroisoquinolin-1(2H)-one (I-161)

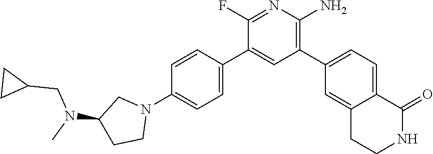 [SMILES: CN(CC1CC1)[C@@H]1CCN(c2ccc(-c3cc(-c4ccc5c(c4)CCNC5=O)c(N)nc3F)cc2)C1]

Step 1: tert-butyl (R)-3-((cyclopropylmethyl)(methyl)amino)pyrrolidine-1-carboxylate

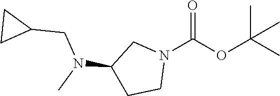 [SMILES: CN(CC1CC1)[C@@H]1CCN(C(=O)OC(C)(C)C)C1]

A mixture of tert-butyl (S)-3-((methylsulfonyl)oxy)pyrrolidine-1-carboxylate (0.290 g, 1.09 mmol), 1-cyclopropyl-N-methylmethanamine (0.233 g, 2.73 mmol) and N,N-diisopropylethylamine (0.476 mL, 2.73 mmol) was heated to 70° C. in a sealed tube for 18 h and then 85° C. for 48 h then at RT for 2 d. The vial was unsealed and diluted with CH₂Cl₂. The mixture was loaded onto Celite® and purified by flash chromatography (0.5-9.5% MeOH/DCM+0.5% NH₄OH; ninhydrin stain) to afford the product (0.727 mmol, 67%) as a clear oil. LCMS: [M+H]⁺=255.37.

Step 2: (R)—N-(cyclopropylmethyl)-N-methylpyrrolidin-3-amine

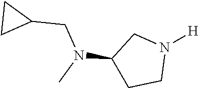

TFA (1.39 mL, 18.2 mmol) was added to a stirring solution of tert-butyl (R)-3-((cyclopropylmethyl)(methyl)amino)pyrrolidine-1-carboxylate (0.185 g, 0.727 mmol) in CH₂Cl₂ (2 ml) at RT. The reaction was allowed to stir at RT for 18 h. A catch and release protocol with SCX-2 silica gel afforded the product (157 mg, 140%). LCMS: [M+H]⁺=155.26.

Step 3: (R)-1-(4-chlorophenyl)-N-(cyclopropylmethyl)-N-methylpyrrolidin-3-amine

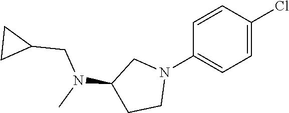 [SMILES: CN(CC1CC1)[C@@H]1CCN(c2ccc(Cl)cc2)C1]

A procedure analogous to Example 108, Step 1 using (R)—N-(cyclopropylmethyl)-N-methylpyrrolidin-3-amine (0.120 g, 0.778 mmol), 1-bromo-4-chlorobenzene (0.179 g, 0.934 mmol), tris(dibenzylideneacetone)dipalladium (0) (0.021 g, 0.023 mmol), 4,5-bis(diphenylphosphino)-9,9-dimethylxanthene (0.041 g, 0.070 mmol) and Cs₂CO₃ (0.760 g, 2.33 mmol) afforded the product (103 mg, 50%). LCMS: [M+H]⁺=265.01.

Step 4: (R)-6-(2-amino-5-(4-(3-((cyclopropylmethyl)(methyl)amino)pyrrolidin-1-yl)phenyl)-6-fluoropyridin-3-yl)-3,4-dihydroisoquinolin-1(2H)-one

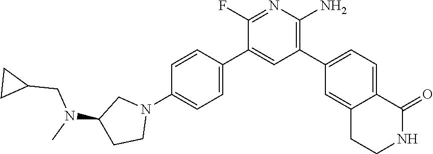 [SMILES: CN(CC1CC1)[C@@H]1CCN(c2ccc(-c3cc(-c4ccc5c(c4)CCNC5=O)c(N)nc3F)cc2)C1]

A procedure analogous to Example 43, Step 5 using 6-(2-amino-6-fluoro-5-(4,4,5,5-tetramethyl-1,3,2-dioxaborolan-2-yl)pyridin-3-yl)-3,4-dihydroisoquinolin-1(2H)-one (0.075 g, 0.196 mmol), (R)-1-(4-chlorophenyl)-N-(cyclopropylmethyl)-N-methylpyrrolidin-3-amine (0.054 g, 0.205 mmol) and XPhos Pd G2 (0.015 g, 0.020 mmol) and aqueous K₃PO₄ (0.376 mL of a 1.3 M solution, 0.489 mmol) afforded the title compound (22 mg, 23%). ¹H NMR (500 MHz, DMSO-d₆) δ 7.8-7.9 (m, 2H), 7.44 (d, J=10.1 Hz, 1H), 7.3-7.4 (m, 2H), 7.25 (d, J=7.9 Hz, 2H), 6.47 (d, 2H, J=8.8 Hz), 5.99 (s, 2H), 3.3-3.4 (m, 5H), 3.1-3.2 (m, 1H), 2.9-3.0 (m, 2H), 2.84 (br t, J=6.5 Hz, 2H), 2.19 (s, 3H), 2.0-2.1 (m, 1H), 1.7-1.8 (m, 1H), 0.7-0.8 (m, 1H), 0.3-0.4 (m, 2H), 0.0-0.0 (m, 2H); LCMS: [M+H]⁺=486.2.

### Example 162: 6-(2-amino-6-fluoro-5-(4-(3-(isobutyl(methyl)amino)pyrrolidin-1-yl)phenyl)pyridin-3-yl)-3,4-dihydroisoquinolin-1(2H)-one (I-162)

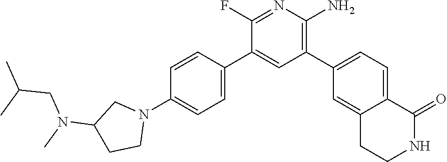 [SMILES: CC(C)CN(C)C1CCN(c2ccc(-c3cc(-c4ccc5c(c4)CCNC5=O)c(N)nc3F)cc2)C1]

Step 1: tert-butyl 3-(N-methylisobutyramido)pyrrolidine-1-carboxylate

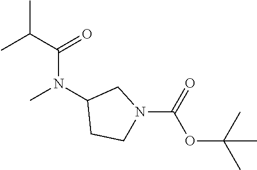 [SMILES: CC(C)C(=O)N(C)C1CCN(C(=O)OC(C)(C)C)C1]

Isobutyryl chloride (0.156 mL, 1.50 mmol) was dropwise to a vial containing a solution of 1-Boc-3-Methylaminopyrrolidine (0.250 g, 1.25 mmol) and Et₃N (0.209 mL, 1.50 mmol) in CH₂Cl₂ (5 mL) at RT. The reaction was allowed to stir at RT overnight. The reaction mixture was concentrated onto Celite® and purified by flash chromatography (0.5-9.5% DCM/MeOH+0.5% NH₄OH; ninhydrin) to afford the product (368 mg, quant. yield). LCMS: [M+H]⁺=215.03.

Step 2: tert-butyl 3-(isobutyl(methyl)amino)pyrrolidine-1-carboxylate

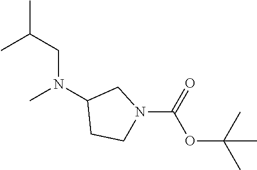 [SMILES: CC(C)CN(C)C1CCN(C(=O)OC(C)(C)C)C1]

Borane dimethyl sulfide complex solution, 2 M in THF (2.04 mL, 4.08 mmol) was added carefully to a cooled (0° C.) solution of tert-butyl 3-(N-methylisobutyramido)pyrrolidine-1-carboxylate (0.368 g, 1.36 mmol) in THF (6 mL) while stirring under nitrogen. The ice bath was removed and the reaction mixture was heated to 60° C. for 4 h. After cooling to RT the reaction was quenched with the dropwise addition of saturated aqueous NaHCO₃ (q) (evolution of gas and exothermic). The reaction was diluted with CH₂Cl₂ and water and the product was isolated by catch and release with SCX-2 silica gel to afford the product (274 mg, 79%) as a clear oil. LCMS: [M+H]⁺=257.41.

Step 3: N-isobutyl-N-methylpyrrolidin-3-amine

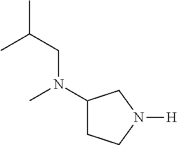

A procedure analogous to Example 61, Step 2 using TFA (2.05 mL, 26.8 mmol) and tert-butyl 3-(isobutyl(methyl)amino)pyrrolidine-1-carboxylate (0.275 g, 1.07 mmol) afforded the product (1.17 mmol, quant. yield). LCMS [M+H]⁺=157.22.

Step 4: 1-(4-chlorophenyl)-N-isobutyl-N-methylpyrrolidin-3-amine

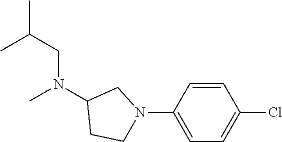 [SMILES: CC(C)CN(C)C1CCN(c2ccc(Cl)cc2)C1]

A procedure analogous to Example 108, Step 1 using N-isobutyl-N-methylpyrrolidin-3-amine (0.170 g, 1.09 mmol), 1-bromo-4-chlorobenzene (0.250 g, 1.31 mmol), tris(dibenzylideneacetone)dipalladium (0) (0.030 g, 0.033 mmol), 4,5-bis(diphenylphosphino)-9,9-dimethylxanthene (0.057 g, 0.098 mmol) and Cs₂CO₃ (1.06 g, 3.26 mmol) afforded the title compound (0.322 mmol, 30%). LCMS:

Step 5: 6-(2-amino-6-fluoro-5-(4-(3-(isobutyl(methyl)amino)pyrrolidin-1-yl)phenyl)pyridin-3-yl)-3,4-dihydroisoquinolin-1(2H)-one

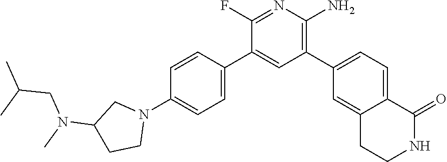 [SMILES: CC(C)CN(C)C1CCN(c2ccc(-c3cc(-c4ccc5c(c4)CCNC5=O)c(N)nc3F)cc2)C1]

A procedure analogous to Example 43, Step 5 using 6-(2-amino-6-fluoro-5-(4,4,5,5-tetramethyl-1,3,2-dioxaborolan-2-yl)pyridin-3-yl)-3,4-dihydroisoquinolin-1(2H)-one (0.075 g, 0.196 mmol), 1-(4-chlorophenyl)-N-isobutyl-N-methylpyrrolidin-3-amine (0.055 g, 0.205 mmol) and XPhos Pd G2 (0.015 g, 0.020 mmol) and aqueous K₃PO₄ (0.376 mL of a 1.3 M solution, 0.489 mmol) afforded the title compound (31 mg, 33%). ¹H NMR (500 MHz, DMSO-d₆) δ 7.8-7.9 (m, 2H), 7.48 (d, J=10.3 Hz, 1H), 7.4-7.4 (m, 2H), 7.29 (d, J=7.7 Hz, 2H), 6.52 (d, J=8.8 Hz, 2H), 6.03 (s, 2H), 3.3-3.4 (m, 5H), 3.1-3.2 (m, 1H), 3.0-3.0 (m, 2H), 2.88 (t, J=6.5 Hz, 2H), 2.0-2.1 (m, 6H), 1.6-1.8 (m, 2H), 0.80 (dd, J=6.6, 1.5 Hz, 6H); LCMS: [M+H]⁺=488.5.

### Example 163: 6-(2-amino-6-fluoro-5-(4-(4-fluoropiperidin-1-yl)phenyl)pyridin-3-yl)-3,4-dihydroisoquinolin-1(2H)-one (I-163)

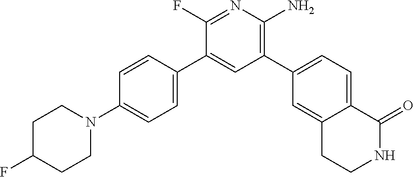 [SMILES: Nc1nc(F)c(-c2ccc(N3CCC(F)CC3)cc2)cc1-c1ccc2c(c1)CCNC2=O]

Step 1: 1-(4-chlorophenyl)-4-fluoropiperidine

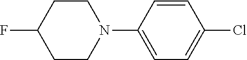 [SMILES: FC1CCN(c2ccc(Cl)cc2)CC1]

A procedure analogous to Example 108, Step 1 using 4-fluoropiperidine hydrochloride (0.182 g, 1.31 mmol), 1-bromo-4-chlorobenzene (0.250 g, 1.31 mmol), tris(dibenzylideneacetone)dipalladium (0) (0.036 g, 0.039 mmol), 4,5-bis(diphenylphosphino)-9,9-dimethylxanthene (0.068 g, 0.118 mmol) and Cs₂CO₃ (1.49 g, 4.57 mmol) afforded the product (253 mg, 91%). LCMS: [M+H]⁺=214.05.

Step 2: 6-(2-amino-6-fluoro-5-(4-(4-fluoropiperidin-1-yl)phenyl)pyridin-3-yl)-3,4-dihydroisoquinolin-1(2H)-one

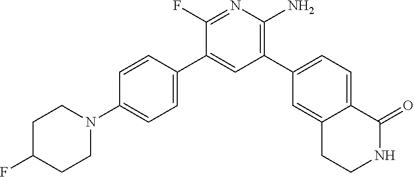 [SMILES: Nc1nc(F)c(-c2ccc(N3CCC(F)CC3)cc2)cc1-c1ccc2c(c1)CCNC2=O]

A procedure analogous to Example 43, Step 5 using 6-(2-amino-6-fluoro-5-(4,4,5,5-tetramethyl-1,3,2-dioxaborolan-2-yl)pyridin-3-yl)-3,4-dihydroisoquinolin-1(2H)-one (0.075 g, 0.196 mmol), 1-(4-chlorophenyl)-4-fluoropiperidine (0.044 g, 0.205 mmol), XPhos Pd G2 (0.015 g, 0.020 mmol) and aqueous K₃PO₄ (0.376 mL of a 1.3 M solution, 0.489 mmol) afforded the title compound (9.0 mg, 11%) as a tan powder. ¹H NMR (500 MHz, DMSO-d₆) δ 7.9-8.0 (m, 2H), 7.59 (d, J=10.1 Hz, 1H), 7.4-7.5 (m, 2H), 7.41 (d, J=7.8 Hz, 2H), 7.01 (d, J=8.9 Hz, 2H), 6.18 (s, 2H), 4.8-4.9 (m, 1H), 3.4-3.4 (m, 5H), 3.1-3.2 (m, 2H), 2.96 (t, J=6.5 Hz, 2H), 1.9-2.0 (m, 2H), 1.7-1.9 (m, 2H); LCMS: [M+H]⁺=435.5.

### Example 164: 6-(2-amino-6-fluoro-5-(4-(1-isopropyl-3-methylpyrrolidin-3-yl)phenyl)pyridin-3-yl)-3,4-dihydroisoquinolin-1(2H)-one (I-164)

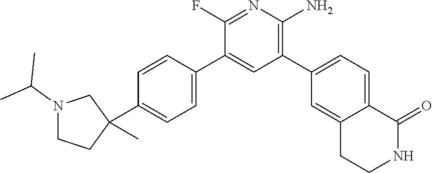 [SMILES: CC(C)N1CCC(C)(c2ccc(-c3cc(-c4ccc5c(c4)CCNC5=O)c(N)nc3F)cc2)C1]

Step 1: 3-(4-chlorophenyl)-1-isopropyl-3-methylpyrrolidine

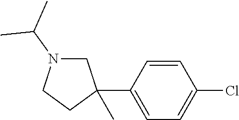 [SMILES: CC(C)N1CCC(C)(c2ccc(Cl)cc2)C1]

A solution of 3-(4-chlorophenyl)-3-methylpyrrolidine·HCl (120 mg, 0.517 mmol), Et₃N (0.14 mL, 1.0 mmol) and acetone (0.077 mL, 1.0 mmol) in CH₂Cl₂ (14 mL) was stirred for 2 h at rt before NaBH(OAc)₃ (274 mg, 1.29 mmol) was added in one portion at RT. Stirring was continued at that temperature for 1 d and saturated aq NaHCO₃, was added. The organic layer was separated and concentrated to afford crude product that was used without further purification.

Step 2. 6-(2-amino-6-fluoro-5-(4-(1-isopropyl-3-methylpyrrolidin-3-yl)phenyl)pyridin-3-yl)-3,4-dihydroisoquinolin-1(2H)-one

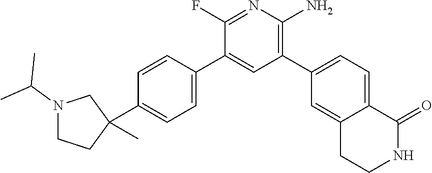 [SMILES: CC(C)N1CCC(C)(c2ccc(-c3cc(-c4ccc5c(c4)CCNC5=O)c(N)nc3F)cc2)C1]

A procedure similar to Example 1, General Method A, heating a sealed and degassed mixture of K₃PO₄ (161 mg, 0.757 mmol), XPhos-Pd-G2 (20 mg, 0.025 mmol), 6-(2-amino-6-fluoro-5-(4,4,5,5-tetramethyl-1,3,2-dioxaborolan-2-yl)pyridin-3-yl)-3,4-dihydroisoquinolin-1(2H)-one (116 mg, 0.303 mmol), 3-(4-chlorophenyl)-1-isopropyl-3-methylpyrrolidine (50% of the material from Step 1, 60 mg, 0.25 mmol), H₂O (6 mL) and MeCN (9 mL) a microwave reactor at 100° C. for 1.5 h, afforded the title compound as a pale yellow solid (30 mg, 26% yield for 2 steps). ¹H NMR (500 MHz, DMSO-d₆) δ 7.95 (br s, 1H), 7.91 (d, J=7.8 Hz, 1H), 7.64 (d, J=10.2 Hz, 1H), 7.43-7.58 (m, 4H), 7.32-7.43 (m, 2H), 6.30 (s, 2H), 3.39-3.49 (m, 2H), 2.96 (br t, J=6.4 Hz, 1H), 2.30-3.25 (br m, 4H), 2.07-2.22 (m, 1H), 1.85-1.99 (m, 1H), 1.30-1.61 (m, 1H), 1.18-1.40 (m, 4H), 0.97-1.17 (br s, 6H); LCMS: [M+H]⁺=459.53.

### Example 165: 6-(2-amino-6-fluoro-5-(4-((1S,5R)-3-(2-methoxyethyl)-3-azabicyclo[3.1.0]hexan-1-yl)phenyl)pyridin-3-yl)-3,4-dihydroisoquinolin-1(2H)-one (I-165)

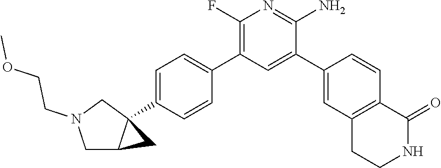 [SMILES: COCCN1C[C@@H]2C[C@]2(c2ccc(-c3cc(-c4ccc5c(c4)CCNC5=O)c(N)nc3F)cc2)C1]

Step 1: 6-(5-(4-((1S,5R)-3-azabicyclo[3.1.0]hexan-1-yl)phenyl)-2-amino-6-fluoropyridin-3-yl)-3,4-dihydroisoquinolin-1(2H)-one

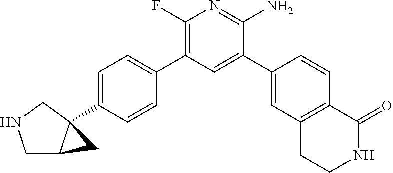 [SMILES: Nc1nc(F)c(-c2ccc([C@@]34CNC[C@@H]3C4)cc2)cc1-c1ccc2c(c1)CCNC2=O]

A procedure analogous to that of Example 43, Step 5 using (1S,5R)-1-(4-bromophenyl)-3-azabicyclo[3.1.0]hexane (60 mg, 0.252 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (126 mg, 0.328 mmol) afforded the product as a beige solid (110 mg, 95% yield based on purity of 90%); LCMS: [M+H]⁺=415.52.

Step 2: Preparation on 6-(2-amino-6-fluoro-5-(4-((1S,5R)-3-(2-methoxyethyl)-3-azabicyclo[3.1.0]hexan-1-yl)phenyl)pyridin-3-yl)-3,4-dihydroisoquinolin-1(2H)-one)

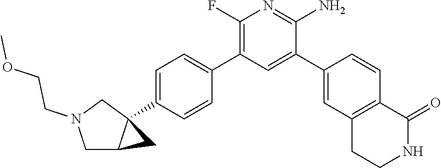 [SMILES: COCCN1C[C@@H]2C[C@]2(c2ccc(-c3cc(-c4ccc5c(c4)CCNC5=O)c(N)nc3F)cc2)C1]

A procedure analogous to that of Example 90, Step 2 using 6-(5-(4-((1S,5R)-3-azabicyclo[3.1.0]hexan-1-yl)phenyl)-2-amino-6-fluoropyridin-3-yl)-3,4-dihydroisoquinolin-1(2H)-one (50 mg, 0.109 mmol) and 2-bromoethyl methyl ether (0.031 ml, 0.326 mmol) overnight at RT. The reaction mixture concentrated onto Celite®. Silica gel chromatography (eluting with CH₂Cl₂ containing 0-4% MeOH and 0-0.4% NH₄OH), followed by reverse phase chromatography (C18; 0-30% acetonitrile/water) afforded the title compound as an off white solid (19 mg, 35%). ¹H NMR (500 MHz, DMSO-d₆) δ 8.02-8.02 (m, 1H), 7.92-7.96 (m, 1 H), 7.88-7.92 (m, 1H), 7.58-7.64 (m, 1H), 7.41-7.48 (m, 4H), 7.15-7.21 (m, 2H), 6.27 (s, 2H), 3.44-3.47 (m, 2H), 3.29-3.32 (m, 1H), 3.23-3.26 (m, 3H), 3.15-3.18 (m, 2H), 3.02-3.07 (m, 1H), 2.92-2.98 (m, 2H), 2.61-2.66 (m, 2H), 2.55-2.59 (m, 1H), 2.41-2.48 (m, 1H), 1.75-1.82 (m, 1H), 1.29-1.33 (m, 1H), 0.72-0.78 (m, 1H); LCMS: [M+H]⁺=473.62.

### Example 166: 6-(2-amino-6-chloro-5-(4-(1-(2-methoxyethyl)piperidin-4-yl)phenyl)pyridin-3-yl)-3,4-dihydroisoquinolin-1(2H)-one (I-166)

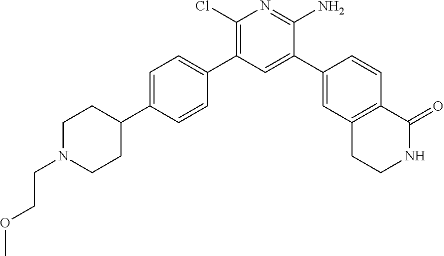 [SMILES: COCCN1CCC(c2ccc(-c3cc(-c4ccc5c(c4)CCNC5=O)c(N)nc3Cl)cc2)CC1]

Step 1: tert-butyl 4-(4-(6-amino-2-chloro-5-(1-oxo-1,2,3,4-tetrahydroisoquinolin-6-yl)pyridin-3-yl)phenyl)piperidine-1-carboxylate

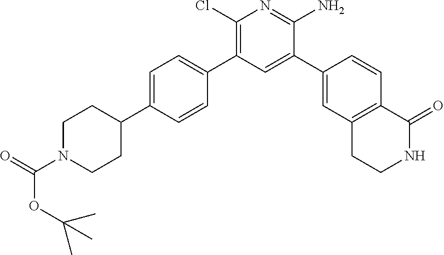 [SMILES: CC(C)(C)OC(=O)N1CCC(c2ccc(-c3cc(-c4ccc5c(c4)CCNC5=O)c(N)nc3Cl)cc2)CC1]

A procedure analogous to that of Example 43, Step 5 using 6-(2-amino-5-bromo-6-chloropyridin-3-yl)-3,4-dihydroisoquinolin-1(2H)-one (50 mg, 0.142 mmol) and 4-(N-Boc-piperidino)phenylboronic acid pinacol ester (71.4 mg, 0.184 mmol) afforded the product as an off white solid (66 mg, 87%). LCMS: [M+H]⁺=533.60.

Step 2: 6-(2-amino-6-chloro-5-(4-(piperidin-4-yl)phenyl)pyridin-3-yl)-3,4-dihydroisoquinolin-1(2H)-one

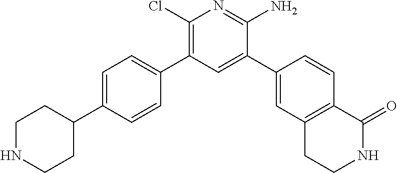 [SMILES: Nc1nc(Cl)c(-c2ccc(C3CCNCC3)cc2)cc1-c1ccc2c(c1)CCNC2=O]

A mixture of tert-butyl 4-(4-(6-amino-2-chloro-5-(1-oxo-1,2,3,4-tetrahydroisoquinolin-6-yl)pyridin-3-yl)phenyl)piperidine-1-carboxylate (66 mg, 0.124 mmol) in MeOH (3.5 mL) with HCl solution 4.0 M in dioxane (0.619 mL, 2.48 mmol) at RT for 2.5 h. The reaction mixture was concentrated and filtered through a Waters PoraPak CX column to afford the title compound as a white powder (52 mg, 97%); LCMS: [M+H]⁺=433.5.

Step 3: 6-(2-amino-6-chloro-5-(4-(1-(2-methoxyethyl)piperidin-4-yl)phenyl)pyridin-3-yl)-3,4-dihydroisoquinolin-1(2H)-one

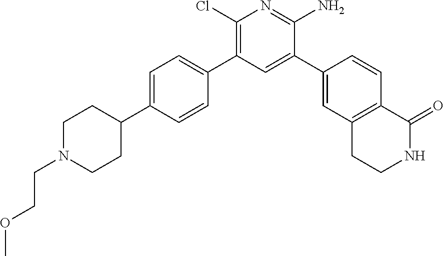 [SMILES: COCCN1CCC(c2ccc(-c3cc(-c4ccc5c(c4)CCNC5=O)c(N)nc3Cl)cc2)CC1]

A procedure analogous to that of Example 90, Step 2 stirring 6-(2-amino-6-chloro-5-(4-(piperidin-4-yl)phenyl)pyridin-3-yl)-3,4-dihydroisoquinolin-1(2H)-one (52 mg, 0.120 mmol), and 2-bromoethyl methyl ether (0.034 ml, 0.360 mmol) overnight at RT afforded the title compound as an off white powder (36.5 mg, 59%). ¹H NMR (500 MHz, DMSO-d₆) δ 7.9-8.0 (m, 1H), 7.9-7.9 (m, 1H), 7.4-7.5 (m, 2H), 7.4-7.4 (m, 3H), 7.3-7.3 (m, 2H), 6.3-6.4 (m, 2H), 3.5-3.5 (m, 2H), 3.4-3.4 (m, 3H), 3.26 (s, 3H), 3.0-3.1 (m, 2H), 2.9-3.0 (m, 2H), 2.5-2.6 (m, 2H), 2.0-2.3 (m, 2H), 1.7-1.8 (m, 2H), 1.6-1.7 (m, 2H); LCMS: [M+H]⁺=491.54.

### Example 167: 6-(2-amino-6-fluoro-5-(4-(4-hydroxy-1-(2-methoxyethyl)piperidin-4-yl)phenyl)pyridin-3-yl)-3,4-dihydroisoquinolin-1(2H)-one (I-167)

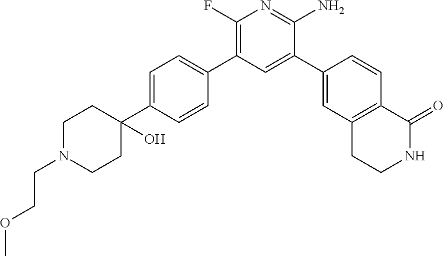 [SMILES: COCCN1CCC(O)(c2ccc(-c3cc(-c4ccc5c(c4)CCNC5=O)c(N)nc3F)cc2)CC1]

Step 1: tert-butyl 4-(4-(6-amino-2-fluoro-5-(1-oxo-1,2,3,4-tetrahydroisoquinolin-6-yl)pyridin-3-yl)phenyl)-4-hydroxypiperidine-1-carboxylate

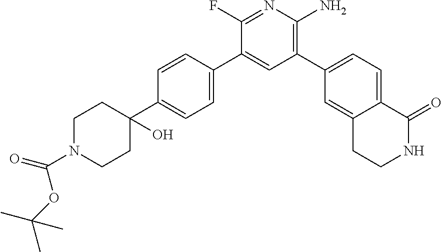 [SMILES: CC(C)(C)OC(=O)N1CCC(O)(c2ccc(-c3cc(-c4ccc5c(c4)CCNC5=O)c(N)nc3F)cc2)CC1]

A procedure analogous to Example 43, Step 5 using tert-butyl 4-(4-chlorophenyl)-4-hydroxypiperidine-1-carboxylate (50 mg, 0.160 mmol), 6-(2-amino-6-fluoro-5-(4,4,5,5-tetramethyl-1,3,2-dioxaborolan-2-yl)pyridin-3-yl)-3,4-dihydroisoquinolin-1(2H)-one (86 mg, 0.225 mmol) K₃PO₄ (102 mg, 0.481 mmol), XPhos Pd G2 (16.40 mg, 0.021 mmol), 1-butanol (4 mL) and water (0.600 mL) afforded the product as a pale yellow solid (75 mg, 88%). LCMS: [M+H]⁺=533.60.

Step 2: 6-(2-Amino-6-fluoro-5-(4-(4-hydroxypiperidin-4-yl)phenyl)pyridin-3-yl)-3,4-dihydroisoquinolin-1(2H)-one

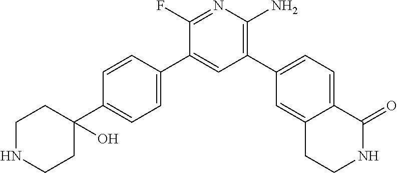 [SMILES: Nc1nc(F)c(-c2ccc(C3(O)CCNCC3)cc2)cc1-c1ccc2c(c1)CCNC2=O]

A solution of 4.0 M HCl in dioxane (0.704 ml, 2.82 mmol) was added to a solution of tert-butyl 4-(4-(6-amino-2-fluoro-5-(1-oxo-1,2,3,4-tetrahydroisoquinolin-6-yl)pyridin-3-yl)phenyl)-4-hydroxypiperidine-1-carboxylate (0.035 mL, 0.141 mmol) in MeOH (4 mL). The reaction mixture was stirred at RT for 2.5 h then concentrated to dryness. The residue was passed through a Waters PoraPak CX column to afford the title compound as a white powder (74 mg, 83% yield based on purity of 68%); LCMS: [M+H]⁺=415.45.

Step 3: 6-(2-amino-6-fluoro-5-(4-(4-hydroxy-1-(2-methoxyethyl)piperidin-4-yl)phenyl)pyridin-3-yl)-3,4-dihydroisoquinolin-1(2H)-one

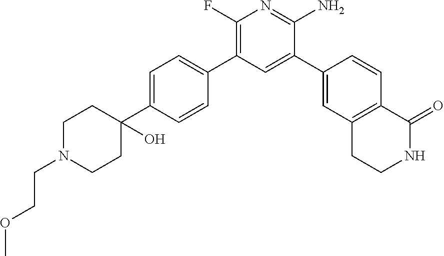 [SMILES: COCCN1CCC(O)(c2ccc(-c3cc(-c4ccc5c(c4)CCNC5=O)c(N)nc3F)cc2)CC1]

A procedure analogous to that of Example 90, Step 2 stirring 6-(2-amino-6-fluoro-5-(4-(4-hydroxypiperidin-4-yl)phenyl)pyridin-3-yl)-3,4-dihydroisoquinolin-1(2H)-one (74 mg, 0.116 mmol) and 2-bromoethyl methyl ether (0.033 mL, 0.349 mmol) at RT overnight afforded the title compound as an off white powder (30 mg, 50%). ¹H NMR (500 MHz, DMSO-d₆) δ 7.93-7.98 (m, 1H), 7.89-7.93 (m, 1H), 7.62-7.68 (m, 1H), 7.45-7.55 (m, 6H), 6.24-6.36 (m, 2H), 4.77-4.88 (m, 1H), 3.45-3.49 (m, 2H), 3.40-3.44 (m, 2H), 3.36-3.37 (m, 2H), 3.29-3.32 (m, 2H), 3.22-3.26 (m, 3H), 2.93-3.00 (m, 2H), 2.64-2.73 (m, 2H), 2.41-2.49 (m, 2H), 1.88-1.99 (m, 2H), 1.54-1.64 (m, 2H); LCMS: [M+H]⁺=491.54.

### Example 168: 6-(2-amino-6-chloro-5-(4-(4-(2,2-difluoroethyl)piperazin-1-yl)phenyl)pyridin-3-yl)-3,4-dihydroisoquinolin-1(2H)-one (I-168)

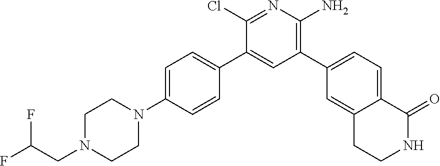 [SMILES: Nc1nc(Cl)c(-c2ccc(N3CCN(CC(F)F)CC3)cc2)cc1-c1ccc2c(c1)CCNC2=O]

Step 1: tert-butyl 4-(4-(6-amino-2-chloro-5-(1-oxo-1,2,3,4-tetrahydroisoquinolin-6-yl)pyridin-3-yl)phenyl)piperazine-1-carboxylate

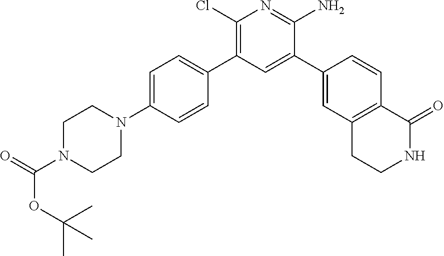 [SMILES: CC(C)(C)OC(=O)N1CCN(c2ccc(-c3cc(-c4ccc5c(c4)CCNC5=O)c(N)nc3Cl)cc2)CC1]

A procedure analogous to that of Example 43, step 5, using 6-(2-amino-5-bromo-6-chloropyridin-3-yl)-3,4-dihydroisoquinolin-1(2H)-one (50 mg, 0.142 mmol) and tert-butyl 4-[4-(4,4,5,5-tetramethyl-1,3,2-dioxaborolan-2-yl)phenyl]tetrahydro-1(2H)-pyrazinecarboxylate (71.6 mg, 0.184 mmol) afforded the product as a pale yellow solid (58 mg, 77%). LCMS: [M+H]⁺=534.60.

Step 2: 6-(2-amino-6-chloro-5-(4-(piperazin-1-yl)phenyl)pyridin-3-yl)-3,4-dihydroisoquinolin-1(2H)-one

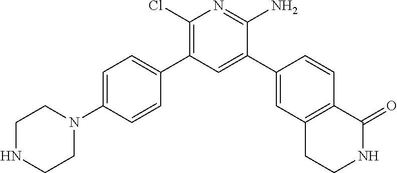 [SMILES: Nc1nc(Cl)c(-c2ccc(N3CCNCC3)cc2)cc1-c1ccc2c(c1)CCNC2=O]

A procedure analogous to that of Example 167, Step 2 stirring tert-butyl 4-(4-(6-amino-2-chloro-5-(1-oxo-1,2,3,4-tetrahydroisoquinolin-6-yl)pyridin-3-yl)phenyl)piperazine-1-carboxylate (58 mg, 0.109 mmol) in MeOH (3.5 mL) with HCl solution (4.0 M in dioxane, 0.543 mL, 2.17 mmol) overnight at RT. The reaction mixture was concentrated and filtered through a Waters PoraPak CX column to afford the title compound as a white powder (53 mg, 96% yield base on purity of 85%); LCMS: [M+H]⁺=434.44.

Step 3: 6-(2-amino-6-chloro-5-(4-(4-(2,2-difluoroethyl)piperazin-1-yl)phenyl)pyridin-3-yl)-3,4-dihydroisoquinolin-1(2H)-one

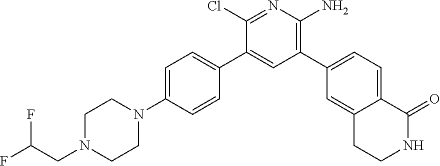 [SMILES: Nc1nc(Cl)c(-c2ccc(N3CCN(CC(F)F)CC3)cc2)cc1-c1ccc2c(c1)CCNC2=O]

A procedure analogous to that of Example 90, Step 2 heating 6-(2-amino-6-chloro-5-(4-(piperazin-1-yl)phenyl)pyridin-3-yl)-3,4-dihydroisoquinolin-1(2H)-one (53 mg, 0.104 mmol, purity 85%) and 2,2-difluoroethyl trifluoromethanesulfonate (0.041 ml, 0.311 mmol) at 80° C. for 3 h afforded the title compound as an off white powder (33 mg, 61%). ¹H NMR (500 MHz, DMSO-d₆) δ 7.94-7.99 (m, 1H), 7.89-7.94 (m, 1H), 7.45-7.51 (m, 2H), 7.37-7.40 (m, 1H), 7.30-7.36 (m, 2H), 6.97-7.03 (m, 2H), 6.09-6.34 (m, 3H), 3.42-3.45 (m, 2H), 3.18-3.23 (m, 4H), 2.94-3.01 (m, 2H), 2.77-2.87 (m, 2H), 2.67-2.74 (m, 4H); LCMS: [M+H]⁺=498.56.

### Example 169: 6-(2-amino-6-chloro-5-(4-(4-isopropylpiperazin-1-yl)phenyl)pyridin-3-yl)-3,4-dihydroisoquinolin-1(2H)-one (I-169)

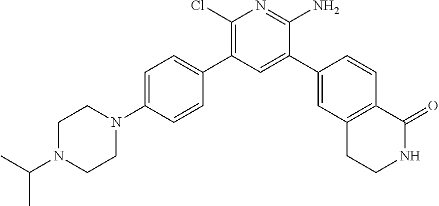 [SMILES: CC(C)N1CCN(c2ccc(-c3cc(-c4ccc5c(c4)CCNC5=O)c(N)nc3Cl)cc2)CC1]

A procedure analogous to that of Example 43, step 5 using 6-(2-amino-5-bromo-6-chloropyridin-3-yl)-3,4-dihydroisoquinolin-1(2H)-one (40 mg, 0.113 mmol), 4-(4-Isopropylpiperazinyl)phenylboronic acid, pinacol ester (48.7 mg, 0.147 mmol) afforded the title compound as a white powder (31 mg, 55%). ¹H NMR (500 MHz, DMSO-d₆) δ ppm 7.92-7.98 (m, 1H), 7.86-7.92 (m, 1H), 7.45-7.49 (m, 1H), 7.43-7.45 (m, 1H), 7.35-7.38 (m, 1H), 7.28-7.34 (m, 2H), 6.93-7.01 (m, 2H), 6.19-6.28 (m, 2H), 3.39-3.43 (m, 2H), 3.13-3.21 (m, 4H), 2.92-2.99 (m, 2H), 2.66-2.74 (m, 1H), 2.56-2.65 (m, 4H), 0.98-1.06 (m, 6H); LCMS: [M+H]⁺=476.63.

### Example 170: 6-(6′-amino-2′-fluoro-5-(4-isopropylpiperazin-1-yl)-[2,3′-bipyridin]-5′-yl)-7-fluoro-3,4-dihydroisoquinolin-1(2H)-one (I-170)

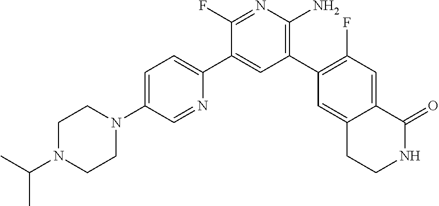 [SMILES: CC(C)N1CCN(c2ccc(-c3cc(-c4cc5c(cc4F)C(=O)NCC5)c(N)nc3F)nc2)CC1]

Step 1: 1-(6-chloropyridin-3-yl)-4-isopropylpiperazine

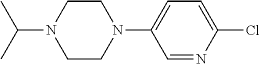 [SMILES: CC(C)N1CCN(c2ccc(Cl)nc2)CC1]

A 20 mL vial was charged with 5-bromo-2-chloropyridine (500 mg, 2.60 mmol), 1-isopropylpiperazine (366 mg, 2.86 mmol), Pd₂dba₃ (71 mg, 0.078 mmol), Xantphos (135 mg, 0.234 mmol) and Cs₂CO₃ (2540 mg, 7.79 mmol). The vial was sealed with a cap and septum and then the reaction vessel was evacuated and backfilled with nitrogen. PhMe (15 mL) was added and the reaction vessel was evacuated and backfilled with nitrogen an additional time. The reaction was heated in oil bath at 80° C. for 4 h and then at 100° C. for 19 h. The reaction mixture was concentrated and purified by flash chromatography (25 g SiO₂, using MeOH in CH₂Cl₂) to afford the product as a red-orange gum (514 mg, 77% yield based on purity of 94%). LCMS: [M+H]⁺=240.10.

Step 2: 6-(6′-Amino-2′-fluoro-5-(4-isopropylpiperazin-1-yl)-[2,3′-bipyridin]-5′-yl)-7-fluoro-3,4-dihydroisoquinolin-1(2H)-one

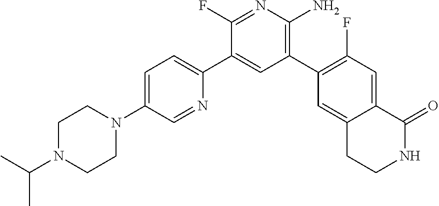 [SMILES: CC(C)N1CCN(c2ccc(-c3cc(-c4cc5c(cc4F)C(=O)NCC5)c(N)nc3F)nc2)CC1]

A procedure similar to Example 1, General method A by heating a sealed, degassed mixture of 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (51 mg, 0.13 mmol, 19.3 mg/mL) in 1,4-dioxane (2.6 mL), K₃PO₄ (81 mg, 0.380 mmol), 1-(6-chloropyridin-3-yl)-4-isopropylpiperazine (32.3 mg, 0.127 mmol, 94% purity), CH₃CN (9 mL), H₂O (6 mL) and XPhos-Pd-G2 (10 mg, 0.013 mmol) in a microwave reactor at 100° C. for 1.5 h afforded after workup and purification the title compound as an off white solid (18 mg, 29% yield based on purity of 97%). ¹H NMR (500 MHz, DMSO-d₆) δ 8.25 (d, J=2.8 Hz, 1H), 8.04 (br s, 1H), 7.95-8.02 (m, 1H), 7.55 (d, J=10.2 Hz, 1H), 7.50-7.53 (m, 1H), 7.33 (d, J=7.1 Hz, 1H), 7.30 (dd, J=8.9, 2.8 Hz, 1H), 6.31 (s, 2H), 3.32-3.38 (m, 2H), 3.06-3.16 (m, 4H), 2.83-2.90 (m, 2H), 2.56-2.67 (m, 1H), 2.48-2.55 (m, 4H), 0.94 (br d, J=6.4 Hz, 6H). LCMS: [M+H]⁺=479.54.

### Example 171: 6-(2-amino-5-(4-(6,6-difluoro-3-isopropyl-3-azabicyclo[3.1.0]hexan-1-yl)phenyl)-6-fluoropyridin-3-yl)-3,4-dihydroisoquinolin-1(2H)-one(1-171)

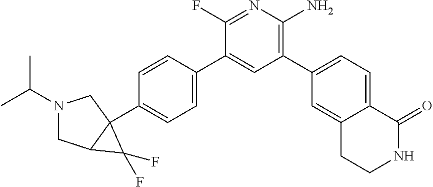 [SMILES: CC(C)N1CC2C(F)(F)C2(c2ccc(-c3cc(-c4ccc5c(c4)CCNC5=O)c(N)nc3F)cc2)C1]

Step 1: tert-butyl 3-(4-bromophenyl)-2,5-dihydro-1H-pyrrole-1-carboxylate

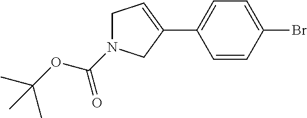 [SMILES: CC(C)(C)OC(=O)N1CC=C(c2ccc(Br)cc2)C1]

A procedure similar to Example 1, General Method A heating a degassed, sealed mixture of 1-bromo-4-iodobenzene (500 mg, 1.77 mmol), 1-Boc-2,5-dihydro-1H-pyrrole-3-boronic acid, pinacol ester (521 mg, 1.77 mmol), Pd(PPh₃)₂Cl₂ (124 mg, 0.177 mmol), MeCN (12 mL), Na₂CO₃ (438 mg, 3.53 mmol) and H₂O (7 mL) in a microwave reactor at 80° C. for 45 min afforded the product as a white solid (426 mg, 74%). LCMS: [M−t−Bu+2H]⁺=268.17/270.20.

Step 2: 1-(4-bromophenyl)-6,6-difluoro-3-azabicyclo[3.1.0]hexane

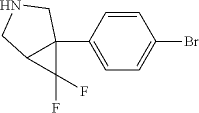 [SMILES: FC1(F)C2CNCC21c1ccc(Br)cc1]

An oven-dried microwave vial was charged with n-Bu4NBr (4.18 mg, 0.013 mmol), tert-butyl 3-(4-bromophenyl)-2,5-dihydro-1H-pyrrole-1-carboxylate (70 mg, 0.22 mmol) and anh. PhMe (2 mL). The sealed vial was filled with Ar and BrCF₂SiMe₃ (66 mg, 0.32 mmol) was added at RT. After briefly stirring at RT, the reaction was heated in an oil bath at 80° C. for 1.5 h and then at 100° C. for 19 h. The reaction was cooled to RT, quenched by an addition of MeOH and concentrated under reduced pressure to afford the product. LCMS: [M+H]⁺=274.19/276.15.

Step 3: 6-(2-amino-5-(4-(6,6-difluoro-3-azabicyclo[3.1.0]hexan-1-yl)phenyl)-6-fluoropyridin-3-yl)-3,4-dihydroisoquinolin-1(2H)-one

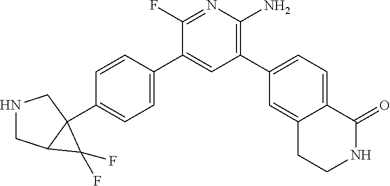 [SMILES: Nc1nc(F)c(-c2ccc(C34CNCC3C4(F)F)cc2)cc1-c1ccc2c(c1)CCNC2=O]

A procedure similar to Example 1, General Method A using 1-(4-bromophenyl)-6,6-difluoro-3-azabicyclo[3.1.0]hexane (59 mg, 0.22 mmol), DME (10 mL), H₂O (5 mL), PdCl₂dppf (16 mg, 0.022 mmol), 6-(2-amino-6-fluoro-5-(4,4,5,5-tetramethyl-1,3,2-dioxaborolan-2-yl)pyridin-3-yl)-3,4-dihydroisoquinolin-1(2H)-one (116 mg, 0.302 mmol) and Cs₂CO₃ (211 mg, 0.648 mmol) afforded the product as a tan solid (60 mg, 55% yield for 2 steps, based on purity of 89%). LCMS: [M+H]⁺=451.48.

Step 4: 6-(2-amino-5-(4-(6,6-difluoro-3-isopropyl-3-azabicyclo[3.1.0]hexan-1-yl)phenyl)-6-fluoropyridin-3-yl)-3,4-dihydroisoquinolin-1(2H)-one

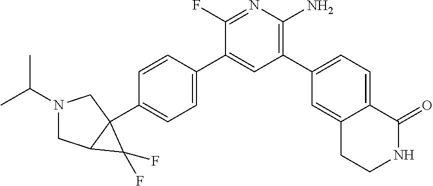 [SMILES: CC(C)N1CC2C(F)(F)C2(c2ccc(-c3cc(-c4ccc5c(c4)CCNC5=O)c(N)nc3F)cc2)C1]

An anhydrous CH₂Cl₂ (20 mL) solution of 6-(2-amino-5-(4-(6,6-difluoro-3-azabicyclo[3.1.0]hexan-1-yl)phenyl)-6-fluoropyridin-3-yl)-3,4-dihydroisoquinolin-1(2H)-one (60 mg, 0.12 mmol, 89% purity), acetone (0.018 mL, 0.24 mmol) and glacial acetic acid (0.014 mL, 0.24 mmol) was stirred at RT for 15 min before NaBH(OAc)₃ (62.8 mg, 0.296 mmol) was added in one portion. Stirring was continued at RT for 2 d. MeOH was added, the reaction mixture was concentrated under reduced pressure and purified by flash chromatography (25 g SiO₂, using CH₂Cl₂/MeOH/aq NH₄OH 89/10/1 in CH₂Cl₂) followed by a filtration through a Waters PoraPak CX column to afford the title compound as an off-white solid (26 mg, 43% yield based on purity of 96%). ¹H NMR (500 MHz, DMSO-d₆) δ 7.86-7.89 (m, 1H), 7.84 (d, J=8.0 Hz, 1H), 7.59 (d, J=10.2 Hz, 1H), 7.47 (br d, J=7.7 Hz, 2H), 7.36-7.43 (m, 2H), 7.29 (br d, J=8.2 Hz, 2H), 6.27 (s, 2H), 3.48-3.55 (m, 1H), 3.31-3.37 (m, 2H), 3.11-3.18 (m, 1H), 2.88 (br t, J=6.4 Hz, 3H), 2.65-2.85 (m, 2H), 2.35-2.41 (m, 1H), 0.94 (br d, J=6.1 Hz, 6H). LCMS: [M+H]⁺=493.46.

### Example 172: 6-(2-amino-6-fluoro-5-(4-(piperazin-1-yl)phenyl)pyridin-3-yl)-7-fluoro-3,4-dihydroisoquinolin-1(2H)-one 2,2,2-trifluoroacetate (1-172)

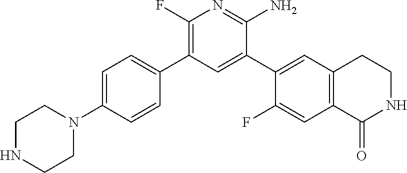 [SMILES: Nc1nc(F)c(-c2ccc(N3CCNCC3)cc2)cc1-c1cc2c(cc1F)C(=O)NCC2]

Step 1: tert-butyl 4-(4-(6-amino-2-fluoro-5-(7-fluoro-1-oxo-1,2,3,4-tetrahydroisoquinolin-6-yl)pyridin-3-yl)phenyl)piperazine-1-carboxylate

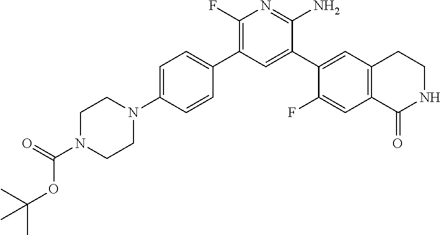 [SMILES: CC(C)(C)OC(=O)N1CCN(c2ccc(-c3cc(-c4cc5c(cc4F)C(=O)NCC5)c(N)nc3F)cc2)CC1]

A procedure similar to General Method A heating a mixture of 6-(2-amino-5-bromo-6-fluoropyridin-3-yl)-7-fluoro-3,4-dihydroisoquinolin-1(2H)-one (320 mg, 0.904 mmol), tert-butyl 4-[4-(4,4,5,5-tetramethyl-1,3,2-dioxaborolan-2-yl)phenyl]tetrahydro-1(2H)-pyrazinecarboxylate (421 mg, 1.08 mmol), Cs₂CO₃ (736 mg, 2.26 mmol) in a 3:1 mixture of DME/H₂O (16 mL) and [1,12-bis(diphenylphosphino)ferrocene]dichloropalladium(II) (66.1 mg, 0.090 mmol) in a microwave apparatus at 90° C. for 2.5 h afforded after workup and purification the product (349 mg, 72%) as a brown solid. LCMS: [M+H]⁺=536.61.

Step 2: 6-(2-amino-6-fluoro-5-(4-(piperazin-1-yl)phenyl)pyridin-3-yl)-7-fluoro-3,4-dihydroisoquinolin-1(2H)-one

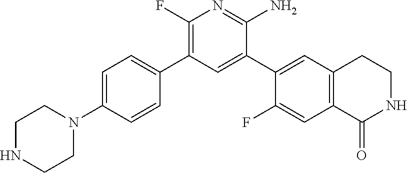 [SMILES: Nc1nc(F)c(-c2ccc(N3CCNCC3)cc2)cc1-c1cc2c(cc1F)C(=O)NCC2]

To a solution of tert-butyl 4-(4-(6-amino-2-fluoro-5-(7-fluoro-1-oxo-1,2,3,4-tetrahydroisoquinolin-6-yl)pyridin-3-yl)phenyl)piperazine-1-carboxylate (349 mg, 0.652 mmol) in CH₂Cl₂ (5.0 mL) was added TFA (1.50 ml, 19.6 mmol). The reaction was stirred at 40° C. for 16 h, then concentrated onto Celite® and purified by reverse phase eluting with 0-100% ACN/H₂O. The desired fractions were collected, concentrated and dried under high vacuum to afford 6-(2-amino-6-fluoro-5-(4-(piperazin-1-yl)phenyl)pyridin-3-yl)-7-fluoro-3,4-dihydroisoquinolin-1(2H)-one-TFA (0.520 mmol, 80%) as a beige solid. ¹H NMR (500 MHz, DMSO-d₆) δ 8.61 (br s, 2H), 8.12 (br s, 1H), 7.61 (dd, J=10.1, 6.8 Hz, 2H), 7.41-7.47 (m, 3H), 7.04 (br d, J=8.8 Hz, 2H), 6.23 (s, 2H), 3.41 (br dd, J=6.7, 2.5 Hz, 2H), 3.37 (br d, J=5.3 Hz, 4H), 3.25 (br d, J=4.8 Hz, 4H), 2.93 (br t, J=6.4 Hz, 2H); LCMS: [M+H]⁺=436.51.

### Example 173: tert-butyl 6-(6-amino-2-fluoro-5-(1-oxo-1,2,3,4-tetrahydroisoquinolin-6-yl)pyridin-3-yl)-4H-spiro[benzo[d][1,3]dioxine-2,4′-piperidine]-1′-carboxylate (1-173)

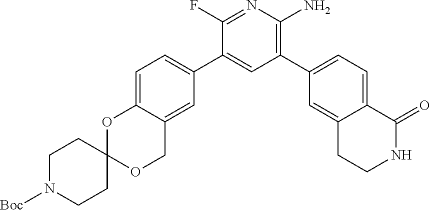 [SMILES: CC(C)(C)OC(=O)N1CCC2(CC1)OCc1cc(-c3cc(-c4ccc5c(c4)CCNC5=O)c(N)nc3F)ccc1O2]

Step 1: tert-butyl 6-(4,4,5,5-tetramethyl-1,3,2-dioxaborolan-2-yl)-4H-spiro[benzo[d][1,3]dioxine-2,4′-piperidine]-1′-carboxylate

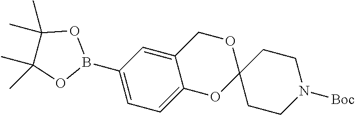 [SMILES: CC(C)(C)OC(=O)N1CCC2(CC1)OCc1cc(B3OC(C)(C)C(C)(C)O3)ccc1O2]

To a solution of bis(pinacolato)diboron (597 mg, 2.350 mmol), tert-butyl 6-bromo-4h-spiro[benzo[d][1,3]dioxine-2,4′-piperidine]-1′-carboxylate (602 mg, 1.567 mmol) in 1,4-dioxane (14 ml) was added KOAc (431 mg, 4.39 mmol) and the reaction mixture was degassed with argon for 15 minutes. Pd(dppf)Cl₂ (172 mg, 0.235 mmol) was added and the reaction mixture was stirred at 90° C. for overnight. The reaction mixture was partitioned into DCM and water and the product was extracted using DCM. The organic phase was dried over MgSO₄ and after filtration and concentration under vacuum, the crude product was dry loaded onto Celite® and purified by flash chromatography (0-30% hexanes/EtOAc) to afford the product (660.0 mg, 98%) as a colorless oil which solidify over time as a white powder which was used for the next step. LCMS: [M-tBu+H]⁺=376.39.

Step 2: tert-butyl 6-(6-amino-2-fluoro-5-(1-oxo-1,2,3,4-tetrahydroisoquinolin-6-yl)pyridin-3-yl)-4H-spiro[benzo[d][1,3]dioxine-2, 4′-piperidine]-1′-carboxylate

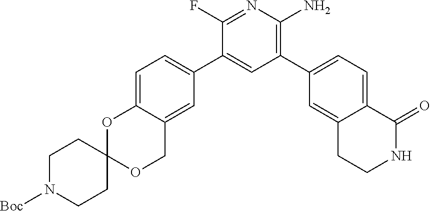 [SMILES: CC(C)(C)OC(=O)N1CCC2(CC1)OCc1cc(-c3cc(-c4ccc5c(c4)CCNC5=O)c(N)nc3F)ccc1O2]

A procedure similar to Example 43, Step 5 using tert-butyl 6-(4,4,5,5-tetramethyl-1,3,2-dioxaborolan-2-yl)-4H-spiro[benzo[d][1,3]dioxine-2,4′-piperidine]-1′-carboxylate (323 mg, 0.750 mmol), tert-butyl 6-(4,4,5,5-tetramethyl-1,3,2-dioxaborolan-2-yl)-4H-spiro[benzo[d][1,3]dioxine-2,4′-piperidine]-1′-carboxylate (323 mg, 0.750 mmol) and 1,4-dioxane (5 mL) Cs₂CO₃ (611 mg, 1.87 mmol) in water (1 mL) and Pd(dppf)Cl₂ (68.6 mg, 0.094 mmol) afforded the title compound (301.9 mg, 0.512 mmol, 82% yield) as a beige powder. ¹H NMR (500 MHz, DMSO-d₆) δ 7.94 (br s, 1H), 7.90 (d, J=7.8 Hz, 1H), 7.60 (d, J=10.1 Hz, 1H), 7.48-7.45 (m, 1H), 7.44 (s, 1H), 7.39-7.34 (m, 1H), 7.28 (s, 1H), 6.89 (d, J=8.6 Hz, 1H), 6.25 (s, 2H), 4.88 (s, 2H), 3.45-3.35 (br m, 6H), 2.97-2.91 (m, 2H), 1.85-1.77 (br m, 4H), 1.41 (s, 9H); [M+H]⁺=561.55.

### Example 174: tert-butyl 6-(6-amino-2-fluoro-5-(1-oxo-1,2,3,4-tetrahydroisoquinolin-6-yl)pyridin-3-yl)-4-oxospiro[chromane-2,4′-piperidine]-1′-carboxylate (1-174)

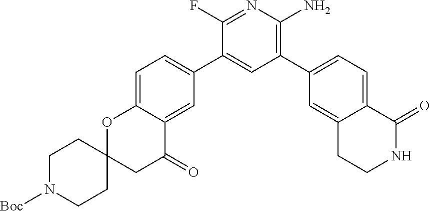 [SMILES: CC(C)(C)OC(=O)N1CCC2(CC1)CC(=O)c1cc(-c3cc(-c4ccc5c(c4)CCNC5=O)c(N)nc3F)ccc1O2]

Step 1: tert-butyl 4-oxo-6-(4,4,5,5-tetramethyl-1,3,2-dioxaborolan-2-yl)spiro[chromane-2,4′-piperidine]-1′-carboxylate

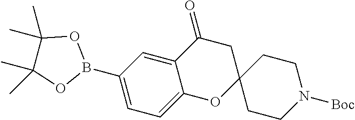 [SMILES: CC(C)(C)OC(=O)N1CCC2(CC1)CC(=O)c1cc(B3OC(C)(C)C(C)(C)O3)ccc1O2]

To a solution of tert-butyl 6-bromo-4-oxospiro[chroman-2,4′-piperidine]-1′-carboxylate (607.9 mg, 1.53 mmol), bis(pinacolato)diboron (584 mg, 2.30 mmol) in 1,4-dioxane (14 ml) was added KOAc (422 mg, 4.30 mmol) and the reaction mixture was degassed with argon and Pd(dppf)Cl₂ (168 mg, 0.230 mmol) was added. The reaction mixture was stirred at 90° C. for 16 h, then partitioned into CH₂Cl₂ and water and the product was extracted using CH₂Cl₂. The organic phase was dried over MgSO₄ and after filtration and concentration under vacuum, the crude product was dry loaded onto Celite® and purified by flash chromatography (0-30% hexanes/EtOAc) to afford the product (596.7 mg, 88%) as a colorless oil which solidify over time as a white powder which was used for the next step. LCMS: [M-Me+H]⁺=429.36.

Step 2: tert-butyl 6-(6-amino-2-fluoro-5-(1-oxo-1,2,3,4-tetrahydroisoquinolin-6-yl)pyridin-3-yl)-4-oxospiro[chromane-2,4′-piperidine]-1′-carboxylate

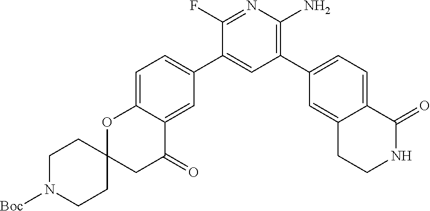 [SMILES: CC(C)(C)OC(=O)N1CCC2(CC1)CC(=O)c1cc(-c3cc(-c4ccc5c(c4)CCNC5=O)c(N)nc3F)ccc1O2]

A procedure analogous to Example 43, Step 5 using tert-butyl 4-oxo-6-(4,4,5,5-tetramethyl-1,3,2-dioxaborolan-2-yl)spiro[chromane-2,4′-piperidine]-1′-carboxylate (318 mg, 0.718 mmol), 6-(2-amino-5-bromo-6-fluoropyridin-3-yl)-3,4-dihydroisoquinolin-1(2H)-one (201 mg, 0.598 mmol) and 1,4-dioxane (3 mL). Then a solution of Cs₂CO₃ (584 mg, 1.79 mmol) in water (1 mL) and Pd(dppf)Cl₂ (65.6 mg, 0.090 mmol) afforded the title compound (359.0 mg, 100%) as a beige powder. ¹H NMR (500 MHz, DMSO-d₆) δ 7.94 (br s, 1H), 7.90 (d, J=7.9 Hz, 1H), 7.86 (s, 1H), 7.81-7.77 (m, 1H), 7.65 (d, J=10.3 Hz, 1H), 7.50-7.44 (m, 2H), 7.14 (d, J=8.6 Hz, 1H), 6.33 (s, 2H), 3.77-3.69 (m, 2H), 3.43-3.37 (m, 2H), 3.25-3.03 (m, 2H), 2.95 (s, 2H), 2.87 (s, 2H), 1.94-1.87 (m, 2H), 1.69-1.60 (m, 2H), 1.40 (s, 9H); [M+H]⁺=573.51.

### Example 175: tert-butyl 6-(6-amino-2-fluoro-5-(1-oxo-1,2,3,4-tetrahydroisoquinolin-6-yl)pyridin-3-yl)spiro[chromane-24′-piperidine]-1′-carboxylate (1-175)

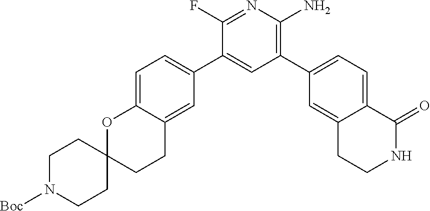 [SMILES: CC(C)(C)OC(=O)N1CCC2(CCc3cc(-c4cc(-c5ccc6c(c5)CCNC6=O)c(N)nc4F)ccc3O2)CC1]

Step 1: tert-butyl 6-(4,4,5,5-tetramethyl-1,3,2-dioxaborolan-2-yl)spiro[chromane-2,4′-piperidine]-1′-carboxylate

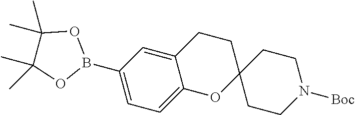 [SMILES: CC(C)(C)OC(=O)N1CCC2(CCc3cc(B4OC(C)(C)C(C)(C)O4)ccc3O2)CC1]

To a solution of bis(pinacolato)diboron (600 mg, 2.364 mmol), tert-butyl 6-bromo-3,4-dihydrospiro[1-benzopyran-2,4′-piperidine]-1′-carboxylate (602.4 mg, 1.576 mmol) in 1,4-dioxane (14 ml) was added KOAc (433 mg, 4.41 mmol) and the reaction mixture was degassed with argon and Pd(dppf)Cl₂ (173 mg, 0.236 mmol) was added. The reaction mixture was stirred at 90° C. for 16 h, then partitioned into CH₂Cl₂ and water and the product was extracted using CH₂Cl₂. The organic phase was dried over MgSO₄ and after filtration and concentration under vacuum, the crude product was dry loaded onto Celite® and purified by flash chromatography (0-20% hexanes/EtOAc) to afford the product (603.9 mg, 89%) as a colorless oil which solidified into a white powder. LCMS: [M+H]⁺=430.41.

Step 2: tert-butyl 6-(6-amino-2-fluoro-5-(1-oxo-1,2,3,4-tetrahydroisoquinolin-6-yl)pyridin-3-yl)spiro[chromane-2,4′-piperidine]-1′-carboxylate

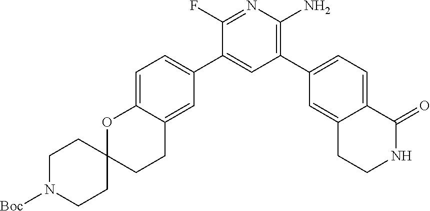 [SMILES: CC(C)(C)OC(=O)N1CCC2(CCc3cc(-c4cc(-c5ccc6c(c5)CCNC6=O)c(N)nc4F)ccc3O2)CC1]

A procedure similar to Example 43, Step 5 using tert-butyl 6-(4,4,5,5-tetramethyl-1,3,2-dioxaborolan-2-yl)spiro[chromane-2,4′-piperidine]-1′-carboxylate (320 mg, 0.746 mmol), 6-(2-amino-5-bromo-6-fluoropyridin-3-yl)-3,4-dihydroisoquinolin-1 (2H)-one (209 mg, 0.622 mmol), 1,4-dioxane (3 mL) Cs₂CO₃ (608 mg, 1.7 mmol) in water (1 mL) and Pd(dppf)Cl₂ (68.2 mg, 0.093 mmol) afforded the title compound (359.0 mg, 98%) as a beige powder. ¹H NMR (500 MHz, DMSO-d₆) δ 7.94 (br s, 1H), 7.90 (d, J=7.9 Hz, 1H), 7.58 (d, J=10.1 Hz, 1H), 7.49-7.42 (m, 2H), 7.28-7.23 (m, 2H), 6.82 (d, J=8.2 Hz, 1H), 6.20 (s, 2H), 3.76-3.65 (m, 2H), 3.40 (br d, J=2.6 Hz, 2H), 3.25-3.03 (m, 2H), 2.95 (s, 2H), 2.76 (s, 2H), 1.84-1.77 (m, 2H), 1.68 (br s, 2H), 1.57-1.49 (m, 2H), 1.41 (s, 10H); [M+H]⁺=559.60.

### Example 176: tert-butyl 4-(4-(6-amino-2-fluoro-5-(1-oxo-1,2,3,4-tetrahydroisoquinolin-6-yl)pyridin-3-yl)-2-cyanophenoxy)piperidine-1-carboxylate (I-176)

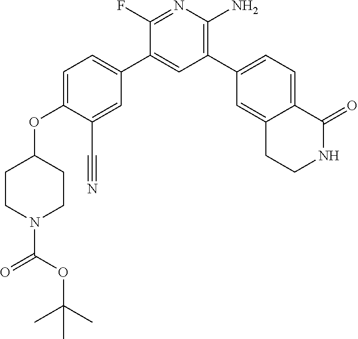 [SMILES: CC(C)(C)OC(=O)N1CCC(Oc2ccc(-c3cc(-c4ccc5c(c4)CCNC5=O)c(N)nc3F)cc2C#N)CC1]

Step 1: tert-butyl 4-(2-cyano-4-(4,4,5,5-tetramethyl-1,3,2-dioxaborolan-2-yl)phenoxy)piperidine-1-carboxylate

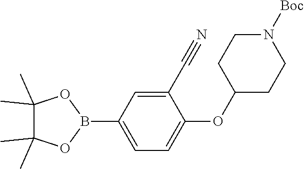 [SMILES: CC(C)(C)OC(=O)N1CCC(Oc2ccc(B3OC(C)(C)C(C)(C)O3)cc2C#N)CC1]

To a solution of tert-butyl 4-(4-bromo-2-cyanophenoxy)piperidine-1-carboxylate (601.2 mg, 1.58 mmol), bis(pinacolato)diboron (601 mg, 2.37 mmol), 1,4-dioxane (14 ml), KOAc (433 mg, 4.42 mmol) and Pd(dppf)Cl₂ (174 mg, 0.237 mmol) afforded the product (757.8 mg, quantitative yield) as a slightly yellow viscous oil. LCMS: [M-tBu+H]⁺=373.30.

Step 2: tert-butyl 4-(4-(6-amino-2-fluoro-5-(1-oxo-1,2,3,4-tetrahydroisoquinolin-6-yl)pyridin-3-yl)-2-cyanophenoxy)piperidine-1-carboxylate

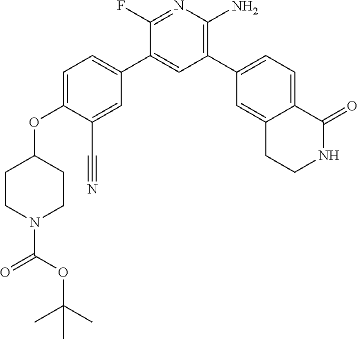 [SMILES: CC(C)(C)OC(=O)N1CCC(Oc2ccc(-c3cc(-c4ccc5c(c4)CCNC5=O)c(N)nc3F)cc2C#N)CC1]

A procedure similar to Example 43, Step 5 using tert-butyl 4-(2-cyano-4-(4,4,5,5-tetramethyl-1,3,2-dioxaborolan-2-yl)phenoxy)piperidine-1-carboxylate (309 mg, 0.721 mmol), 6-(2-amino-5-bromo-6-fluoropyridin-3-yl)-3,4-dihydroisoquinolin-1 (2H)-one (202 mg, 0.601 mmol), 1,4-dioxane (3 ml) and Cs₂CO₃ (587 mg, 1.80 mmol) in water (1 mL) and Pd(dppf)Cl₂ (66.0 mg, 0.090 mmol) afforded the title compound (340.9 mg, 97%) as a white powder. ¹H NMR (500 MHz, DMSO-d₆) δ 7.94 (d, J=2.3 Hz, 2H), 7.91 (d, J=8.1 Hz, 1H), 7.83 (br d, J=9.3 Hz, 1H), 7.71 (d, J=10.3 Hz, 1H), 7.48 (dd, J=7.9, 1.6 Hz, 1H), 7.45 (s, 1H), 7.39 (d, J=9.2 Hz, 1H), 6.42-6.35 (m, 2H), 4.88-4.82 (m, 1H), 3.62-3.54 (m, 2H), 3.44-3.38 (m, 2H), 3.32-3.27 (m, 2H), 2.98-2.92 (m, 2H), 1.96-1.88 (m, 2H), 1.67-1.59 (m, 2H), 1.41 (s, 9H); LCMS: [M+H]⁺=558.39.

### Example 177: tert-butyl 4-(4-(6-amino-2-fluoro-5-(1-oxo-1,2,3,4-tetrahydroisoquinolin-6-yl)pyridin-3-yl)-2-(trifluoromethyl)phenyl)piperazine-1-carboxylate (1-177)

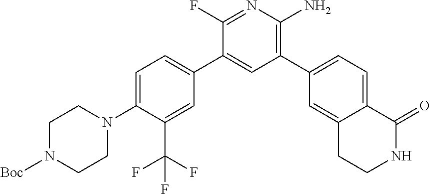 [SMILES: CC(C)(C)OC(=O)N1CCN(c2ccc(-c3cc(-c4ccc5c(c4)CCNC5=O)c(N)nc3F)cc2C(F)(F)F)CC1]

Step 1: tert-butyl 4-(4-(6-amino-5-chloro-2-fluoropyridin-3-yl)-2-(trifluoromethyl)phenyl)piperazine-1-carboxylate

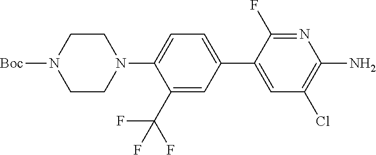 [SMILES: CC(C)(C)OC(=O)N1CCN(c2ccc(-c3cc(Cl)c(N)nc3F)cc2C(F)(F)F)CC1]

In a vial were added tert-butyl 4-(4-(4,4,5,5-tetramethyl-1,3,2-dioxaborolan-2-yl)-2-(trifluoromethyl)phenyl)piperazine-1-carboxylate (516 mg, 1.13 mmol), 5-bromo-3-chloro-6-fluoropyridin-2-amine (255 mg, 1.13 mmol) and 1,4-dioxane (8 mL). Then a solution of Cs₂CO₃ (1105 mg, 3.39 mmol) in water (1 mL) was added at RT. The reaction mixture was degassed under argon and Pd(dppf)Cl₂ (124 mg, 0.170 mmol) was added and the reaction mixture was heated at 90° C. for 16 h. The mixture was dry loaded onto Celite® and purified by flash chromatography (0-10% DCM/MeOH) to afford the product (313.4 mg, 58%) as a grey powder. LCMS: [M+H]⁺=475.33.

Step 2: tert-butyl 4-(4-(6-amino-2-fluoro-5-(1-oxo-1,2,3,4-tetrahydroisoquinolin-6-y)pyridin-3-yl)-2-(trifluoromethyl)phenyl)piperazine-1-carboxylate

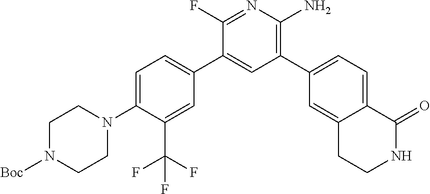 [SMILES: CC(C)(C)OC(=O)N1CCN(c2ccc(-c3cc(-c4ccc5c(c4)CCNC5=O)c(N)nc3F)cc2C(F)(F)F)CC1]

A procedure analogous to that of Example 43, Step 5 using tert-butyl 4-(4-(6-amino-5-chloro-2-fluoropyridin-3-yl)-2-(trifluoromethyl)phenyl)piperazine-1-carboxylate (156.8 mg, 0.330 mmol), 3,4-dihydro-1(2H)-isoquinolinone-6-boronic acid pinacol ester (108 mg, 0.396 mmol) and 1,4-dioxane (4 mL), K₃PO₄ (210 mg, 0.991 mmol) in water (1 mL), and XPhos Pd G2 (26.0 mg, 0.033 mmol) afforded the title compound (133.4 mg, 66%) as a dark beige powder. ¹H NMR (500 MHz, DMSO-d₆) δ 7.98-7.93 (m, 1H), 7.90 (d, J=7.9 Hz, 1H), 7.80 (s, 2H), 7.72 (d, J=10.1 Hz, 1H), 7.61 (d, J=8.3 Hz, 1H), 7.48 (d, J=7.9 Hz, 1H), 7.45 (s, 1H), 6.41 (s, 2H), 3.49-3.38 (m, 6H), 2.97-2.92 (m, 2H), 2.87-2.81 (m, 4H), 1.43 (s, 9H); LCMS: [M+H]⁺=586.53.

### Example 178: 6-(2-amino-5-(4-(((2S,4s,6R)-2,6-dimethylpiperidin-4-yl)oxy)phenyl)-6-fluoropyridin-3-yl)-3,4-dihydroisoquinolin-1(2H)-one (I-178)

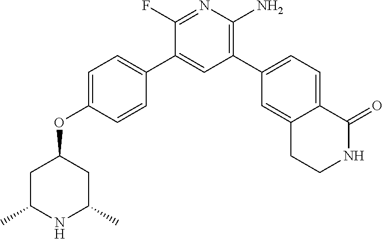 [SMILES: C[C@@H]1C[C@@H](Oc2ccc(-c3cc(-c4ccc5c(c4)CCNC5=O)c(N)nc3F)cc2)C[C@H](C)N1]

Step 1: tert-butyl (2R,4S,6S)-4-(4-bromophenoxy)-2,6-dimethylpiperidine-1-carboxylate

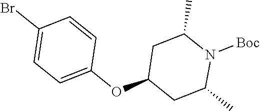 [SMILES: C[C@@H]1C[C@@H](Oc2ccc(Br)cc2)C[C@H](C)N1C(=O)OC(C)(C)C]

A procedure analogous to Example 147, Step 1 using tert-butyl (2R,4r,6S)-rel-4-hydroxy-2,6-dimethylpiperidine-1-carboxylate (510 mg, 2.224 mmol), 4-bromophenol (385 mg, 2.22 mmol), PPh₃ (642 mg, 2.45 mmol) in anhydrous THF (10 mL) and diisopropyl azodicarboxylate (0.525 mL, 2.67 mmol) afforded the product (642.0 mg, 75%) and 4-bromophenol with a ratio of 4:6 and as a slightly yellow oil which was used for the next step. LCMS: [M-tBu+H]⁺=328.38.

Step 2: tert-butyl (2R,4S,6S)-4-(4-(6-amino-2-fluoro-5-(1-oxo-1,2,3,4-tetrahydroisoquinolin-6-yl)pyridin-3-yl)phenoxy)-2,6-dimethylpiperidine-1-carboxylate

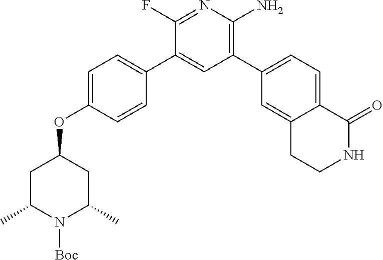 [SMILES: C[C@@H]1C[C@@H](Oc2ccc(-c3cc(-c4ccc5c(c4)CCNC5=O)c(N)nc3F)cc2)C[C@H](C)N1C(=O)OC(C)(C)C]

A procedure analogous to Example 43, Step 5 using tert-butyl (2R,4s,6S)-4-(4-bromophenoxy)-2,6-dimethylpiperidine-1-carboxylate (188 mg, 0.488 mmol), 6-(2-amino-5-bromo-6-fluoropyridin-3-yl)-3,4-dihydroisoquinolin-1(2H)-one (144 mg, 0.376 mmol) and 1,4-dioxane (3 mL), K₃PO₄ (239 mg, 1.13 mmol) in water (1 mL) and XPhos Pd G2 (29.6 mg, 0.038 mmol) afforded the title compound (71.8 mg, 34% yield) as a dark oil which was used for the next step. LCMS: [M+H]⁺=561.55.

Step 3: 6-(2-amino-5-(4-(((2R,4S,6S)-2,6-dimethylpiperidin-4-yl)oxy)phenyl)-6-fluoropyridin-3-yl)-3,4-dihydroisoquinolin-1(2H)-one

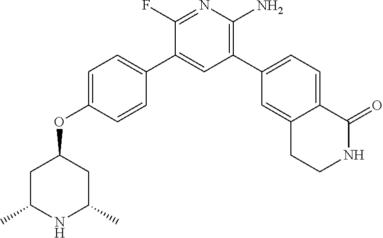 [SMILES: C[C@@H]1C[C@@H](Oc2ccc(-c3cc(-c4ccc5c(c4)CCNC5=O)c(N)nc3F)cc2)C[C@H](C)N1]

A procedure analogous to Example 144, Step 3 using tert-butyl (2R,4s,6S)-4-(4-(6-amino-2-fluoro-5-(1-oxo-1,2,3,4-tetrahydroisoquinolin-6-yl)pyridin-3-yl)phenoxy)-2,6-dimethylpiperidine-1-carboxylate (71.8 mg, 0.128 mmol), TFA (2 mL, 26.1 mmol) and CH₂Cl₂ (4 mL) afforded the title compound (29.0 mg, 44%) as a beige powder. ¹H NMR (500 MHz, DMSO-d₆) δ 7.95 (br s, 1H), 7.90 (d, J=7.9 Hz, 1H), 7.60 (d, J=10.1 Hz, 1H), 7.52-7.42 (m, 4H), 7.04 (br d, J=8.7 Hz, 2H), 6.25 (s, 2H), 4.89-4.83 (m, 1H), 3.46-3.37 (m, 5H), 2.98-2.92 (m, 2H), 2.10-2.03 (m, 2H), 1.76-1.65 (m, 2H), 1.24 (br d, J=6.4 Hz, 8H); LCMS: [M+H]⁺=461.49.

### Example 179: 6-(2-amino-6-fluoro-5-(3-fluoro-4-(piperidin-4-yloxy)phenyl)pyridin-3-yl)-3,4-dihydroisoquinolin-1(2H)-one (I-179)

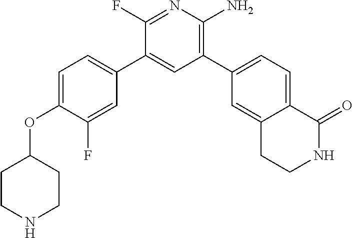 [SMILES: Nc1nc(F)c(-c2ccc(OC3CCNCC3)c(F)c2)cc1-c1ccc2c(c1)CCNC2=O]

Step 1: tert-butyl 4-(4-bromo-2-fluorophenoxy)piperidine-1-carboxylate

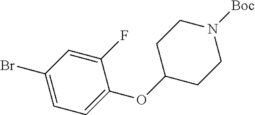 [SMILES: CC(C)(C)OC(=O)N1CCC(Oc2ccc(Br)cc2F)CC1]

A procedure analogous to Example 147, Step 1 using 4-bromo-2-fluorophenol (517 mg, 2.71 mmol), tert-butyl 4-hydroxy-1-piperidinecarboxylate (495 mg, 2.459 mmol), PPh₃ (710 mg, 2.71 mmol), THF (10 mL) and diethyl azodicarboxylate 40 wt % in toluene (2.24 mL, 4.92 mmol) afforded the title compound (764.0 mg, 83%) and 4-bromo-2-fluorophenol with a ratio of 84:16 and as a slightly yellow oil which was used without further purification for the next step. LCMS: [M−Me+H]⁺=361.27.

Step 2: tert-butyl 4-(4-(6-amino-2-fluoro-5-(1-oxo-1,2,3,4-tetrahydroisoquinolin-6-yl)pyridin-3-yl)-2-fluorophenoxy)piperidine-1-carboxylate

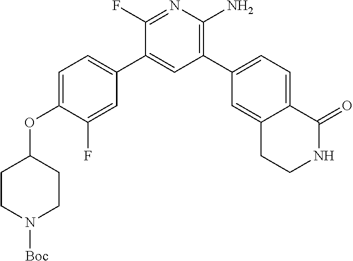 [SMILES: CC(C)(C)OC(=O)N1CCC(Oc2ccc(-c3cc(-c4ccc5c(c4)CCNC5=O)c(N)nc3F)cc2F)CC1]

A procedure similar to Example 43, Step 5 using tert-butyl 4-(4-bromo-2-fluorophenoxy)piperidine-1-carboxylate (175 mg, 0.468 mmol), 6-(2-amino-5-bromo-6-fluoropyridin-3-yl)-3,4-dihydroisoquinolin-1(2H)-one (138 mg, 0.360 mmol), 1,4-dioxane (3 mL), K₃PO₄ (229 mg, 1.08 mmol) in water (1 mL) and XPhos Pd G2 (28.3 mg, 0.036 mmol) afforded the product (49.0 mg, 25%) as a beige powder. LCMS: [M−tBu+H]⁺=495.42.

Step 3: 6-(2-amino-6-fluoro-5-(3-fluoro-4-(piperidin-4-yloxy)phenyl)pyridin-3-yl)-3,4-dihydroisoquinolin-1(2H)-one

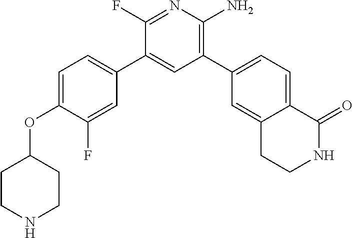 [SMILES: Nc1nc(F)c(-c2ccc(OC3CCNCC3)c(F)c2)cc1-c1ccc2c(c1)CCNC2=O]

A procedure analogous to Example 144, Step 3 using tert-butyl 4-(4-(6-amino-2-fluoro-5-(1-oxo-1,2,3,4-tetrahydroisoquinolin-6-yl)pyridin-3-yl)-2-fluorophenoxy)piperidine-1-carboxylate (49.0 mg, 0.089 mmol), TFA (2 mL, 26.1 mmol) and CH₂Cl₂ (4 mL) afforded the product (18.2 mg, 43%) as a beige powder. ¹H NMR (500 MHz, DMSO-d₆) δ 7.95 (br s, 1H), 7.91 (d, J=7.9 Hz, 1H), 7.66 (d, J=10.1 Hz, 1H), 7.48 (d, J=8.1 Hz, 1H), 7.47-7.40 (m, 2H), 7.34-7.28 (m, 1H), 7.27-7.21 (m, 1H), 6.32 (s, 2H), 4.50-4.40 (m, 1H), 3.44-3.39 (m, 2H), 2.96 (br s, 4H), 2.62-2.53 (m, 2H), 1.97-1.89 (m, 2H), 1.55-1.45 (m, 2H); LCMS: [M+H]⁺=451.41.

### Example 180: (R)-6-(2-amino-6-fluoro-5-(4-(piperidin-3-yloxy)phenyl)pyridin-3-yl)-7-fluoro-3,4-dihydroisoquinolin-1(2H)-one (I-180)

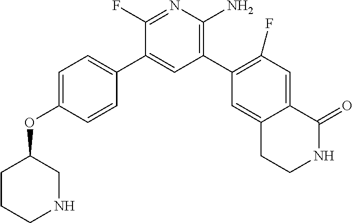 [SMILES: Nc1nc(F)c(-c2ccc(O[C@@H]3CCCNC3)cc2)cc1-c1cc2c(cc1F)C(=O)NCC2]

Step 1: tert-butyl (R)-3-(4-bromophenoxy)piperidine-1-carboxylate

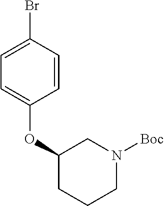 [SMILES: CC(C)(C)OC(=O)N1CCC[C@@H](Oc2ccc(Br)cc2)C1]

To a suspension of NaH, 60% in mineral oil (2.59 g, 64.8 mmol) in DMF (20 mL) was added tert-butyl-(3R)-3-hydroxypiperidine-1-carboxylate (2.5 g, 12.42 mmol) and 1-bromo-4-fluorobenzene (5.46 ml, 49.7 mmol) at RT. The mixture was stirred at 90° C. for 4 hours and then evaporated to dryness. The residue is taken up in 50 mL of ice-water and extracted with DCM. The organic extracts were dried over MgSO₄ and after filtration, the solvents were evaporated to dryness, to give the crude material which was dry loaded onto Celite® and purified by flash chromatography (0-30% DCM/MeOH) to afford the product (2.2 g, 50%) as a yellow oil which was used for the next step. LCMS: [M−tBu+H]⁺=302.15.

Step 2: tert-butyl (R)-3-(4-(4,4,5,5-tetramethyl-1,3,2-dioxaborolan-2-yl)phenoxy)piperidine-1-carboxylate

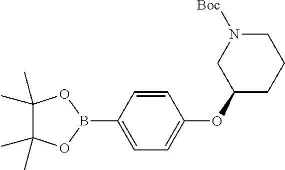 [SMILES: CC(C)(C)OC(=O)N1CCC[C@@H](Oc2ccc(B3OC(C)(C)C(C)(C)O3)cc2)C1]

To a solution of tert-butyl (R)-3-(4-bromophenoxy)piperidine-1-carboxylate (2.2 g, 6.18 mmol), bis(pinacolato)diboron (2.35 g, 9.26 mmol) in 1,4-dioxane (14 ml) was added KOAc (1.70 g, 17.3 mmol) and the reaction mixture was degassed with argon for 15 min. Then Pd(dppf)Cl₂ (0.678 g, 0.926 mmol) was added and the reaction mixture was stirred at 90° C. for 16 h. The reaction mixture was partitioned into DCM and water and the product was extracted using DCM. The organic phase was dried over MgSO₄ and after filtration and concentration under vacuum, the crude product was dry loaded onto Celite® and purified by flash chromatography (0-20% hexanes/EtOAc) to afford the product (2.00 g, 80%) as a colorless oil which solidified to a white powder. LCMS: [M−tBu+H]⁺=348.55.

Step 3: tert-butyl (R)-3-(4-(6-amino-5-chloro-2-fluoropyridin-3-yl)phenoxy)piperidine-1-carboxylate

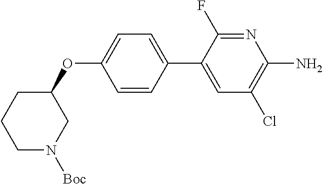 [SMILES: CC(C)(C)OC(=O)N1CCC[C@@H](Oc2ccc(-c3cc(Cl)c(N)nc3F)cc2)C1]

In a vial were added tert-butyl (R)-3-(4-(4,4,5,5-tetramethyl-1,3,2-dioxaborolan-2-yl)phenoxy)piperidine-1-carboxylate (369 mg, 0.916 mmol), 5-bromo-3-chloro-6-fluoropyridin-2-amine (206.4 mg, 0.916 mmol) and 1,4-dioxane (4 mL). Then a solution of Cs₂CO₃ (895 mg, 2.75 mmol) in Water (1 ml) was added at RT and the reaction mixture was degassed under argon for 15 minutes. Then Pd(dppf)Cl₂ (100 mg, 0.137 mmol) was added and the reaction mixture was heated at 90° C. for overnight. The reaction mixture was dry loaded onto Celite® and purified by flash chromatography [0-10% DCM/MeOH] to afford the product (94.1 mg, 24%) as a dark beige powder which was used for the next step. [M−tBu+H]⁺ 366.15.

Step 4: tert-butyl (R)-3-(4-(6-amino-2-fluoro-5-(7-fluoro-1-oxo-1,2,3,4-tetrahydroisoquinolin-6-yl)pyridin-3-yl)phenoxy)piperidine-1-carboxylate

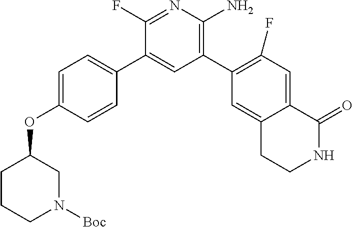 [SMILES: CC(C)(C)OC(=O)N1CCC[C@@H](Oc2ccc(-c3cc(-c4cc5c(cc4F)C(=O)NCC5)c(N)nc3F)cc2)C1]

A procedure analogous to Example 143, Step 5 using tert-butyl (R)-3-(4-(6-amino-5-chloro-2-fluoropyridin-3-yl)phenoxy)piperidine-1-carboxylate (94.1 mg, 0.223 mmol), 7-fluoro-6-(4,4,5,5-tetramethyl-1,3,2-dioxaborolan-2-yl)-3,4-dihydroisoquinolin-1(2H)-one (78 mg, 0.268 mmol), 1,4-dioxane (3 mL), K₃PO₄ (142 mg, 0.669 mmol) in water (1 mL) and XPhos Pd G2 (17.55 mg, 0.022 mmol) afforded the product (35.0 mg, 27%) as a dark beige powder which was used for the next step. LCMS: [M+H]⁺=551.47.

Step 5: rac-(R)-6-(2-amino-6-fluoro-5-(4-(piperidin-3-yloxy)phenyl)pyridin-3-yl)-7-fluoro-3,4-dihydroisoquinolin-1(2H)-one

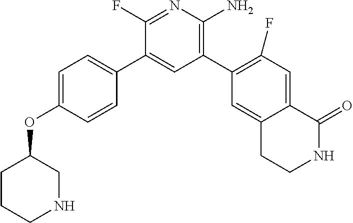 [SMILES: Nc1nc(F)c(-c2ccc(O[C@@H]3CCCNC3)cc2)cc1-c1cc2c(cc1F)C(=O)NCC2]

A procedure analogous to Example 144, Step 3 using tert-butyl (R)-3-(4-(6-amino-2-fluoro-5-(7-fluoro-1-oxo-1,2,3,4-tetrahydroisoquinolin-6-yl)pyridin-3-yl)phenoxy)piperidine-1-carboxylate (35.0 mg, 0.064 mmol), TFA (2 mL, 26.1 mmol) and CH₂Cl₂ (4 mL) afforded the title compound (18.2 mg, 59%) as a beige powder. ¹H NMR (500 MHz, DMSO-d₆) δ 8.10 (br s, 1H), 7.60 (dd, J=10.1, 6.3 Hz, 2H), 7.46-7.40 (m, 3H), 6.98 (d, J=8.8 Hz, 2H), 6.23 (s, 2H), 4.33-4.25 (m, 1H), 3.43-3.38 (m, 2H), 3.16-3.08 (m, 1H), 2.95-2.89 (m, 2H), 2.82-2.75 (m, 1H), 2.58-2.52 (m, 2H), 2.07-1.98 (m, 1H), 1.73-1.64 (m, 1H), 1.56-1.40 (m, 2H); LCMS: [M+H]⁺=451.48.

### Example 181: 6-(2-amino-6-fluoro-5-(4H-spiro[benzo[d][1,3]dioxine-2,4′-piperidin]-6-yl)pyridin-3-yl)-3,4-dihydroisoquinolin-1(2H)-one (I-181)

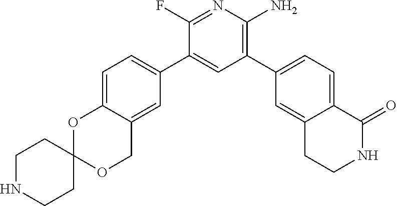 [SMILES: Nc1nc(F)c(-c2ccc3c(c2)COC2(CCNCC2)O3)cc1-c1ccc2c(c1)CCNC2=O]

A procedure analogous to Example 144, Step 3 using tert-butyl 6-(6-amino-2-fluoro-5-(1-oxo-1,2,3,4-tetrahydroisoquinolin-6-yl)pyridin-3-yl)-4H-spiro[benzo[d][1,3]dioxine-2,4′-piperidine]-1′-carboxylate (285 mg, 0.508 mmol), TFA (2 mL, 26.1 mmol) and CH₂Cl₂ (4 mL), TFA (2 mL, 26.1 mmol) afforded the title compound (166.1 mg, 67%) as a beige powder. ¹H NMR (500 MHz, DMSO-d₆) δ 7.94 (br s, 1H), 7.90 (d, J=7.9 Hz, 1H), 7.59 (d, J=10.3 Hz, 1H), 7.49-7.42 (m, 2H), 7.35 (br d, J=8.3 Hz, 1H), 7.26 (s, 1H), 6.86 (d, J=8.6 Hz, 1H), 6.24 (s, 2H), 4.84 (s, 2H), 3.44-3.38 (m, 2H), 2.95 (s, 2H), 2.75 (br s, 4H), 1.74 (br s, 4H); LCMS: [M+H]⁺=461.49.

### Example 182: 6-(6-amino-2-fluoro-5-(1-oxo-1,2,3,4-tetrahydroisoquinolin-6-yl)pyridin-3-yl)spiro[chromane-2,4′-piperidin]-4-one 2,2,2-trifluoroacetate (1-182)

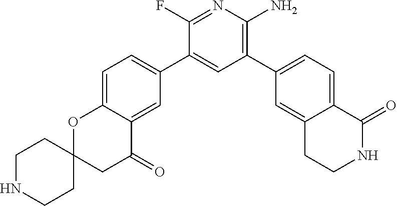 [SMILES: Nc1nc(F)c(-c2ccc3c(c2)C(=O)CC2(CCNCC2)O3)cc1-c1ccc2c(c1)CCNC2=O]

A procedure similar to Example 144, Step 3 using tert-butyl 6-(6-amino-2-fluoro-5-(1-oxo-1,2,3,4-tetrahydroisoquinolin-6-yl)pyridin-3-yl)-4-oxospiro[chromane-2,4′-piperidine]-1′-carboxylate (339 mg, 0.592 mmol), TFA (2 mL, 26.1 mmol) and CH₂Cl₂ (4 mL) afforded the title compound (183.0 mg, 50%) as a white powder. ¹H NMR (500 MHz, DMSO-d₆) δ 8.76-8.32 (m, 2H, TFA), 7.95 (br s, 1H), 7.93-7.87 (m, 2H), 7.86-7.81 (m, 1H), 7.64 (d, J=10.3 Hz, 1H), 7.49-7.43 (m, 2H), 7.20 (d, J=8.7 Hz, 1H), 6.35 (s, 2H), 3.43-3.38 (m, 2H), 3.26-3.19 (m, 2H), 3.18-3.11 (m, 2H), 2.96 (s, 4H), 2.16-2.09 (m, 2H), 1.93-1.82 (m, 2H); LCMS: [M+H]⁺=473.30.

### Example 183: 6-(2-amino-6-fluoro-5-(spiro[chromane-2,4′-piperidin]-6-yl)pyridin-3-yl)-3,4-dihydroisoquinolin-1(2H)-one (I-183)

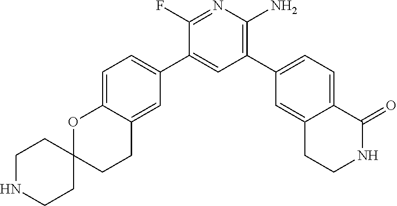 [SMILES: Nc1nc(F)c(-c2ccc3c(c2)CCC2(CCNCC2)O3)cc1-c1ccc2c(c1)CCNC2=O]

A procedure analogous to Example 144, Step 3 using tert-butyl 6-(6-amino-2-fluoro-5-(1-oxo-1,2,3,4-tetrahydroisoquinolin-6-yl)pyridin-3-yl)spiro[chromane-2,4′-piperidine]-1′-carboxylate (339 mg, 0.607 mmol), TFA (2 mL, 26.1 mmol) and CH₂Cl₂ (4 mL) afforded the title compound (225.3 mg, 77%) as a beige powder. ¹H NMR (500 MHz, DMSO-d₆) δ 7.97-7.92 (m, 1H), 7.90 (d, J=7.9 Hz, 1H), 7.57 (d, J=10.1 Hz, 1H), 7.46 (dd, J=1.5, 7.9 Hz, 1H), 7.44 (s, 1H), 7.26-7.21 (m, 2H), 6.79 (d, J=8.8 Hz, 1H), 6.19 (s, 2H), 3.40 (td, J=3.2, 6.5 Hz, 2H), 3.17 (s, 1H), 2.95 (t, J=6.5 Hz, 2H), 2.82 (br s, 2H), 2.74 (s, 2H), 2.70 (br s, 2H), 1.77 (br t, J=6.7 Hz, 2H), 1.61 (br s, 2H), 1.50 (br s, 2H); LCMS: [M+H]⁺=459.38.

### Example 184: 5-(6-amino-2-fluoro-5-(1-oxo-1,2,3,4-tetrahydroisoquinolin-6-yl)pyridin-3-yl)-2-(piperidin-4-yloxy)benzonitrile (I-184)

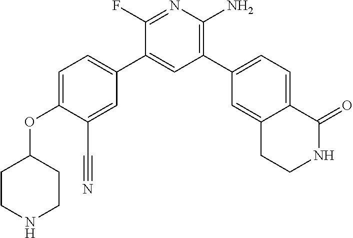 [SMILES: N#Cc1cc(-c2cc(-c3ccc4c(c3)CCNC4=O)c(N)nc2F)ccc1OC1CCNCC1]

A procedure analogous to Example 144, Step 3 using tert-butyl 4-(4-(6-amino-2-fluoro-5-(1-oxo-1,2,3,4-tetrahydroisoquinolin-6-yl)pyridin-3-yl)-2-cyanophenoxy)piperidine-1-carboxylate (320 mg, 0.574 mmol), TFA (2 mL, 26.1 mmol) and CH₂Cl₂ afforded the title compound (159.1 mg, 58%) as a beige powder. ¹H NMR (500 MHz, DMSO-d₆) δ 7.97-7.89 (m, 3H), 7.80 (br d, J=8.9 Hz, 1H), 7.70 (d, J=10.3 Hz, 1H), 7.51-7.47 (m, 1H), 7.45 (s, 1H), 7.35 (d, J=9.0 Hz, 1H), 6.37 (s, 2H), 4.73-4.64 (m, 1H), 3.44-3.38 (m, 2H), 3.01-2.91 (m, 4H), 2.64-2.56 (m, 2H), 2.34-2.03 (m, 1H), 1.97-1.87 (m, 2H), 1.59-1.49 (m, 2H); LCMS: [M+H]⁺=458.18.

### Example 185: 6-(2-amino-6-fluoro-5-(4-(piperazin-1-yl)-3-(trifluoromethyl)phenyl)pyridin-3-yl)-3,4-dihydroisoquinolin-1(2H)-one (I-185)

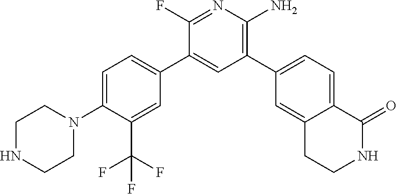 [SMILES: Nc1nc(F)c(-c2ccc(N3CCNCC3)c(C(F)(F)F)c2)cc1-c1ccc2c(c1)CCNC2=O]

A procedure analogous to Example 144, Step 3 using tert-butyl 4-(4-(6-amino-2-fluoro-5-(1-oxo-1,2,3,4-tetrahydroisoquinolin-6-yl)pyridin-3-yl)-2-(trifluoromethyl)phenyl)piperazine-1-carboxylate (115.0 mg, 0.196 mmol), TFA (2 mL, 26.1 mmol) and CH₂Cl₂ afforded the title compound (70.2 mg, 70%) as a beige powder. ¹H NMR (500 MHz, DMSO-d₆) δ 7.95 (br s, 1H), 7.91 (d, J=7.9 Hz, 1H), 7.83 (br d, J=8.4 Hz, 1H), 7.79 (s, 1H), 7.72 (d, J=10.1 Hz, 1H), 7.55 (d, J=8.4 Hz, 1H), 7.51-7.47 (m, 1H), 7.46 (s, 1H), 6.40 (s, 2H), 3.45-3.38 (m, 2H), 2.99-2.93 (m, 2H), 2.82 (br d, J=5.4 Hz, 8H); LCMS: [M+H]⁺=486.32.

### Example 186: 6-(2-amino-6-fluoro-5-(4-((1-(2,2,2-trifluoroethyl)piperidin-4-yl)oxy)phenyl)pyridin-3-yl)-3,4-dihydroisoquinolin-1(2H)-one (I-186)

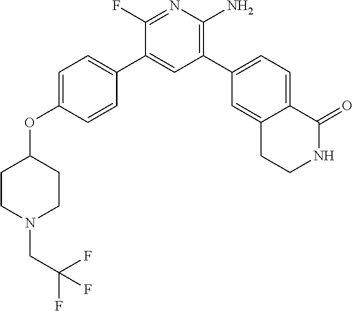 [SMILES: Nc1nc(F)c(-c2ccc(OC3CCN(CC(F)(F)F)CC3)cc2)cc1-c1ccc2c(c1)CCNC2=O]

To a solution of 6-(2-amino-6-fluoro-5-(4-(piperidin-4-yloxy)phenyl)pyridin-3-yl)-3,4-dihydroisoquinolin-1(2H)-one (23.9 mg, 0.055 mmol) and N,N-diisopropylethylamine (0.096 mL, 0.553 mmol) in DMF (1.5 mL) was added 2,2,2-trifluoroethyl trifluoromethanesulfonate (22.3 mg, 0.096 mmol) at RT. The reaction mixture was stirred for 3 hours at RT, then passed through a PoraPak Rxn CX (15cc-2g) cartridge in a catch & elute method by first loading the solution of product in MeOH onto the MeOH-rinsed cartridge and releasing product with 20 mL of a solution of ammonia in MeOH (3N) to afford the crude product. Further purification using flash silica gel chromatography (0-10% MeOH/DCM) to afforded the product (25.8 mg, 86%) as a beige powder. ¹H NMR (500 MHz, DMSO-d₆) δ 7.93 (br s, 1H), 7.90 (d, J=7.9 Hz, 1H), 7.59 (d, J=10.1 Hz, 1H), 7.49-7.42 (m, 4H), 7.00 (d, J=8.9 Hz, 2H), 6.22 (s, 2H), 4.47-4.39 (m, 1H), 3.43-3.37 (m, 2H), 3.19 (q, J=10.2 Hz, 2H), 2.95 (s, 2H), 2.90-2.82 (m, 2H), 2.62-2.54 (m, 2H), 1.98-1.88 (m, 2H), 1.71-1.59 (m, 2H); LCMS: [M+H]⁺=515.51.

### Example 187: 6-(2-amino-6-fluoro-5-(4-((1-(3,3,3-trifluoropropyl)piperidin-4-yl)oxy)phenyl)pyridin-3-yl)-3,4-dihydroisoquinolin-1(2H)-one (I-187)

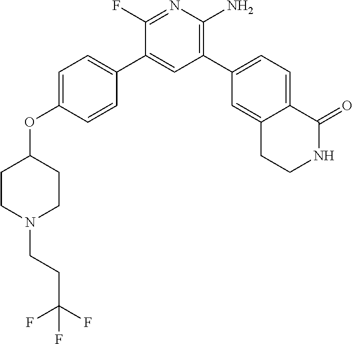 [SMILES: Nc1nc(F)c(-c2ccc(OC3CCN(CCC(F)(F)F)CC3)cc2)cc1-c1ccc2c(c1)CCNC2=O]

A procedure similar to Example 186 was used with 6-(2-amino-6-fluoro-5-(4-(piperidin-4-yloxy)phenyl)pyridin-3-yl)-3,4-dihydroisoquinolin-1(2H)-one (22.3 mg, 0.052 mmol), N,N-diisopropylethylamine (0.090 ml, 0.516 mmol) in DMF(1.5 mL) and addition 1,1,1-trifluoro-3-iodopropane (6.04 μl, 0.052 mmol) at RT. After stirring the reaction mixture for 72 h at RT additional 1,1,1-trifluoro-3-iodopropane (6.04 μl, 0.052 mmol) was added and the mixture was stirred 3 h at RT, then heated at 50° C. for 16 h. A workup and purification similar to Example 186 afforded the title compound (11.9 mg, 42%) as a beige powder. ¹H NMR (500 MHz, DMSO-d₆) δ 7.93 (br s, 1H), 7.90 (d, J=7.9 Hz, 1H), 7.59 (d, J=10.1 Hz, 1H), 7.48-7.43 (m, 4H), 6.99 (d, J=8.8 Hz, 2H), 6.22 (s, 2H), 4.45-4.37 (m, 1H), 3.43-3.38 (m, 2H), 2.98-2.92 (m, 2H), 2.76-2.67 (m, 2H), 2.56-2.51 (m, 2H), 2.48-2.40 (m, 2H), 2.32-2.23 (m, 2H), 1.98-1.90 (m, 2H), 1.67-1.58 (m, 2H); LCMS: [M+H]⁺=529.43.

### Example 188: 6-(2-amino-6-fluoro-5-(4-((1-(4,4,4-trifluorobutyl)piperidin-4-yl)oxy)phenyl)pyridin-3-yl)-3,4-dihydroisoquinolin-1(2H)-one (I-188)

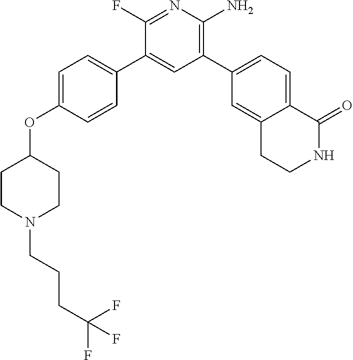 [SMILES: Nc1nc(F)c(-c2ccc(OC3CCN(CCCC(F)(F)F)CC3)cc2)cc1-c1ccc2c(c1)CCNC2=O]

A procedure similar to Example 186 using 6-(2-amino-6-fluoro-5-(4-(piperidin-4-yloxy)phenyl)pyridin-3-yl)-3,4-dihydroisoquinolin-1(2H)-one (22.3 mg, 0.052 mmol) and N,N-diisopropylethylamine (0.090 ml, 0.516 mmol) in DMF (1.5 mL) and 1-bromo-4,4,4-trifluorobutane (17 mg, 0.089 mmol) afforded the title compound (24.7 mg, 84%) as a beige powder. ¹H NMR (500 MHz, DMSO-d₆) δ 7.93 (br s, 1H), 7.90 (d, J=7.9 Hz, 1H), 7.59 (d, J=10.1 Hz, 1H), 7.49-7.42 (m, 4H), 6.99 (d, J=8.8 Hz, 2H), 6.22 (s, 2H), 4.44-4.36 (m, 1H), 3.43-3.37 (m, 2H), 2.98-2.92 (m, 2H), 2.72-2.62 (m, 2H), 2.39-2.33 (m, 2H), 2.30-2.18 (m, 4H), 1.98-1.90 (m, 2H), 1.67-1.58 (m, 4H); LCMS: [M+H]⁺=543.35.

### Example 189: 6-(2-amino-6-fluoro-5-(4-((1-(2-methoxyethyl)piperidin-4-yl)oxy)phenyl)pyridin-3-yl)-3,4-dihydroisoquinolin-1(2H)-one (I-189)

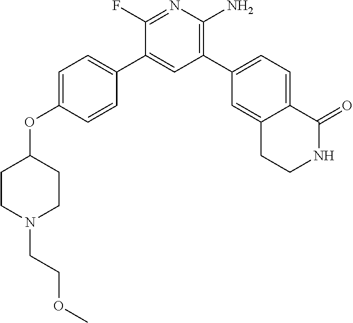 [SMILES: COCCN1CCC(Oc2ccc(-c3cc(-c4ccc5c(c4)CCNC5=O)c(N)nc3F)cc2)CC1]

A procedure similar to Example 186 with 6-(2-amino-6-fluoro-5-(4-(piperidin-4-yloxy)phenyl)pyridin-3-yl)-3,4-dihydroisoquinolin-1(2H)-one (27.4 mg, 0.063 mmol) and N,N-diisopropylethylamine (0.110 ml, 0.634 mmol) in DMF (1.5 ml) and adding 2-bromoethyl methyl ether (5.95 μl, 0.063 mmol) at RT was used. After stirring for 72 h at RT, additional 2-bromoethyl methyl ether (5.95 μl, 0.063 mmol) was added and the mixture was stirred for 3 h at RT. A similar workup and isolation procedure as Example 186 afforded the title compound (20.0 mg, 61%) as a beige powder. ¹H NMR (500 MHz, DMSO-d₆) δ 7.94 (br s, 1H), 7.90 (d, J=7.9 Hz, 1H), 7.59 (d, J=10.1 Hz, 1H), 7.49-7.42 (m, 4H), 6.99 (br d, J=8.7 Hz, 2H), 6.22 (s, 2H), 4.51-4.31 (m, 1H), 3.50-3.43 (m, 2H), 3.40 (br d, J=2.4 Hz, 2H), 3.24 (s, 3H), 2.95 (br t, J=6.5 Hz, 2H), 2.88-2.65 (m, 2H), 2.63-2.51 (m, 2H), 2.42-2.14 (m, 2H), 2.05-1.88 (m, 2H), 1.73-1.56 (m, 2H); LCMS: [M+H]⁺=491.51.

### Example 190: 6-(2-amino-6-fluoro-5-(4-(piperidin-4-yloxy)-3-(trifluoromethyl)phenyl)pyridin-3-yl)-3,4-dihydroisoquinolin-1(2H)-one (I-190)

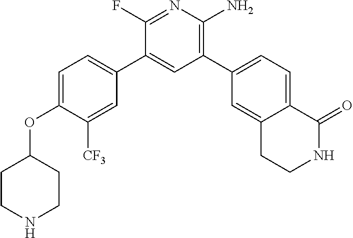 [SMILES: Nc1nc(F)c(-c2ccc(OC3CCNCC3)c(C(F)(F)F)c2)cc1-c1ccc2c(c1)CCNC2=O]

Step 1: tert-butyl 4-(4-bromo-2-(trifluoromethyl)phenoxy)piperidine-1-carboxylate

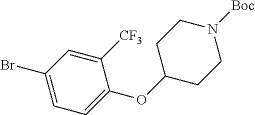 [SMILES: CC(C)(C)OC(=O)N1CCC(Oc2ccc(Br)cc2C(F)(F)F)CC1]

A procedure similar to Example 147, Step 1 using 4-bromo-2-trifluoromethylphenol (765 mg, 3.17 mmol), tert-butyl 4-hydroxy-1-piperidinecarboxylate (638.9 mg, 3.17 mmol), PPh₃ (916 mg, 3.49 mmol) in THF (10 ml) and diisopropyl azodicarboxylate (0.750 ml, 3.81 mmol) afforded the product (0.7131 g, 46%) and 4-bromo-2-(trifluoromethyl)phenol with a ratio of 84:16 and as a colorless oil which was used for the next step. LCMS: [M−Me+H]⁺=411.30.

Step 2: tert-butyl 4-(4-(6-amino-2-fluoro-5-(1-oxo-1,2,3,4-tetrahydroisoquinolin-6-yl)pyridin-3-yl)-2-(trifluoromethyl)phenoxy)piperidine-1-carboxylate

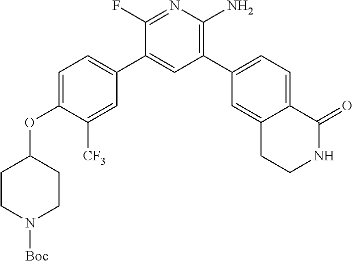 [SMILES: CC(C)(C)OC(=O)N1CCC(Oc2ccc(-c3cc(-c4ccc5c(c4)CCNC5=O)c(N)nc3F)cc2C(F)(F)F)CC1]

A procedure analogous to Example 43, Step 5 using tert-butyl 4-(4-bromo-2-(trifluoromethyl)phenoxy)piperidine-1-carboxylate (350 mg, 0.693 mmol), 6-(2-amino-6-fluoro-5-(4,4,5,5-tetramethyl-1,3,2-dioxaborolan-2-yl)pyridin-3-yl)-3,4-dihydroisoquinolin-1(2H)-one (345 mg, 0.901 mmol), 1,4-dioxane (4 mL), K₃PO₄ (441 mg, 2.08 mmol) in water (1 mL) and XPhos Pd G2 (54.5 mg, 0.069 mmol) afforded the title compound (330.5 mg, 79%) as a beige powder. LCMS: [M+H]⁺=601.50.

Step 3: 6-(2-amino-6-fluoro-5-(4-(piperidin-4-yloxy)-3-(trifluoromethyl)phenyl)pyridin-3-yl)-3,4-dihydroisoquinolin-1(2H)-one

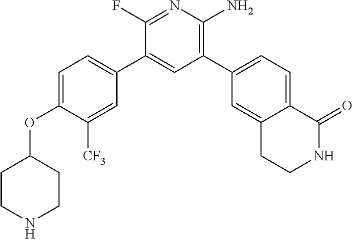 [SMILES: Nc1nc(F)c(-c2ccc(OC3CCNCC3)c(C(F)(F)F)c2)cc1-c1ccc2c(c1)CCNC2=O]

A procedure similar to Example 144, Step 3 using tert-butyl 4-(4-(6-amino-2-fluoro-5-(1-oxo-1,2,3,4-tetrahydroisoquinolin-6-yl)pyridin-3-yl)-2-(trifluoromethyl)phenoxy)piperidine-1-carboxylate (330.5 mg, 0.550 mmol), TFA (2 mL, 26.1 mmol) and CH₂Cl₂ (4 mL) afforded the title compound (178.9 mg, 62%) as a slightly yellow powder which will be used for the next step. ¹H NMR (500 MHz, DMSO-d₆) δ 7.94 (br s, 1H), 7.90 (d, J=7.9 Hz, 1H), 7.77-7.70 (m, 2H), 7.68 (br d, J=10.0 Hz, 1H), 7.47 (br d, J=7.9 Hz, 1H), 7.44 (s, 1H), 7.35 (br d, J=8.7 Hz, 1H), 6.33 (s, 2H), 4.72-4.64 (m, 1H), 3.43-3.37 (m, 2H), 2.98-2.87 (m, 4H), 2.63-2.55 (m, 2H), 1.92-1.85 (m, 2H), 1.57-1.47 (m, 2H); LCMS: [M+H]⁺=501.36.

### Example 191: 6-(5-(1′-acetyl-4H-spiro[benzo[d][1,3]dioxine-2,4′-piperidin]-6-yl)-2-amino-6-fluoropyridin-3-yl)-3,4-dihydroisoquinolin-1(2H)-one (I-191)

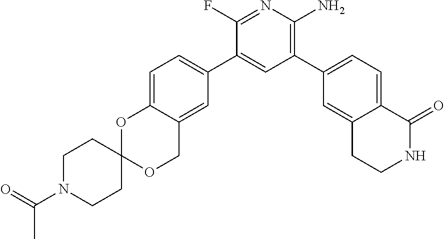 [SMILES: CC(=O)N1CCC2(CC1)OCc1cc(-c3cc(-c4ccc5c(c4)CCNC5=O)c(N)nc3F)ccc1O2]

To a solution of 6-(2-amino-6-fluoro-5-(4H-spiro[benzo[d][1,3]dioxine-2,4′-piperidin]-6-yl)pyridin-3-yl)-3,4-dihydroisoquinolin-1(2H)-one (24.4 mg, 0.053 mmol) and N,N-diisopropylethylamine (0.046 ml, 0.265 mmol) in CH₂Cl₂ (3 mL) was added acetic anhydride (5.01 μl, 0.053 mmol) at RT. The reaction mixture was stirred for 1 h at 23° C. The solvents were removed and the crude mixture was dissolved in MeOH and loaded onto a MeOH washed PoraPak Rxn CX (15cc-2g) cartridge. After washing the cartridge was washed with additional MeOH (3×20 mL), the product was released using 20 mL of a solution of ammonia in MeOH (3N) to afford, after removal of solvent, the title compound (25.9 mg, 92%) as a white powder. ¹H NMR (500 MHz, DMSO-d₆) δ 7.94 (br s, 1H), 7.90 (d, J=7.9 Hz, 1H), 7.60 (d, J=10.1 Hz, 1H), 7.48-7.45 (m, 1H), 7.44 (s, 1H), 7.40-7.35 (m, 1H), 7.29 (s, 1H), 6.90 (d, J=8.6 Hz, 1H), 6.25 (s, 2H), 4.89 (s, 2H), 3.61-3.55 (m, 1H), 3.54-3.48 (m, 3H), 3.42-3.38 (m, 2H), 2.95 (s, 2H), 2.03 (s, 3H), 1.89 (br s, 2H), 1.82-1.74 (m, 2H); LCMS: [M+H]⁺=503.47.

### Example 192: 1′-acetyl-6-(6-amino-2-fluoro-5-(1-oxo-1,2,3,4-tetrahydroisoquinolin-6-yl)pyridin-3-yl)spiro[chromane-2,4′-piperidin]-4-one (I-192)

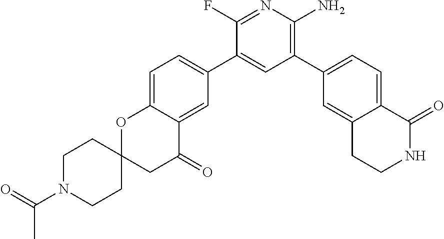 [SMILES: CC(=O)N1CCC2(CC1)CC(=O)c1cc(-c3cc(-c4ccc5c(c4)CCNC5=O)c(N)nc3F)ccc1O2]

A procedure analogous to Example 191 using 6-(6-amino-2-fluoro-5-(1-oxo-1,2,3,4-tetrahydroisoquinolin-6-yl)pyridin-3-yl)spiro[chromane-2,4′-piperidin]-4-one (23.7 mg, 0.050 mmol), N,N-diisopropylethylamine (0.044 ml, 0.251 mmol) in CH₂Cl₂ (3 mL) and acetic anhydride (4.74 μl, 0.050 mmol) afforded the title compound (18.6 mg, 68.5% yield) as a white powder. ¹H NMR (500 MHz, DMSO-d₆) δ 7.94 (br s, 1H), 7.90 (d, J=7.9 Hz, 1H), 7.87 (s, 1H), 7.83-7.77 (m, 1H), 7.65 (d, J=10.3 Hz, 1H), 7.50-7.46 (m, 1H), 7.45 (s, 1H), 7.15 (d, J=8.7 Hz, 1H), 6.33 (s, 2H), 4.16-4.07 (m, 1H), 3.70-3.61 (m, 1H), 3.40 (dt, J=2.4, 6.5 Hz, 3H), 2.95 (br t, J=6.6 Hz, 3H), 2.87 (s, 2H), 2.01 (s, 3H), 1.98-1.87 (m, 2H), 1.79-1.70 (m, 1H), 1.63-1.55 (m, 1H); LCMS: [M+H]⁺=515.51.

### Example 193: 6-(5-(1′-acetylspiro[chromane-2,4′-piperidin]-6-yl)-2-amino-6-fluoropyridin-3-yl)-3,4-dihydroisoquinolin-1(2H)-one (I-193)

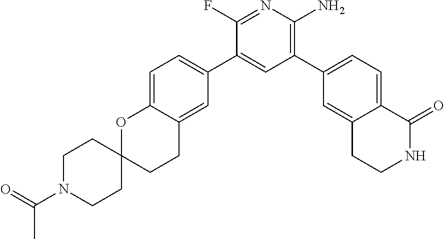 [SMILES: CC(=O)N1CCC2(CCc3cc(-c4cc(-c5ccc6c(c5)CCNC6=O)c(N)nc4F)ccc3O2)CC1]

A procedure analogous to Example 191 using 6-(2-amino-6-fluoro-5-(spiro[chromane-2,4′-piperidin]-6-yl)pyridin-3-yl)-3,4-dihydroisoquinolin-1(2H)-one (25.9 mg, 0.056 mmol) and N,N-diisopropylethylamine (0.049 ml, 0.282 mmol) in CH₂Cl₂ (3 mL) and acetic anhydride (5.34 μl, 0.056 mmol) afforded the title compound (28.0 mg, 94%) as a white powder. ¹H NMR (500 MHz, DMSO-d₆) δ 7.94 (br s, 1H), 7.90 (d, J=7.9 Hz, 1H), 7.58 (d, J=10.1 Hz, 1H), 7.46 (d, J=7.9 Hz, 1H), 7.44 (s, 1H), 7.28 (s, 2H), 6.84 (d, J=8.2 Hz, 1H), 6.20 (s, 2H), 4.13-4.04 (m, 1H), 3.69-3.59 (m, 1H), 3.44-3.37 (m, 3H), 3.06-2.98 (m, 1H), 2.95 (s, 2H), 2.77 (s, 2H), 2.02 (s, 3H), 1.81 (s, 2H), 1.78-1.59 (m, 3H), 1.54-1.44 (m, 1H); LCMS: [M+H]⁺=501.51.

### Example 194: 2-((1-acetylpiperidin-4-yl)oxy)-5-(6-amino-2-fluoro-5-(1-oxo-1,2,3,4-tetrahydroisoquinolin-6-yl)pyridin-3-yl)benzonitrile (I-194)

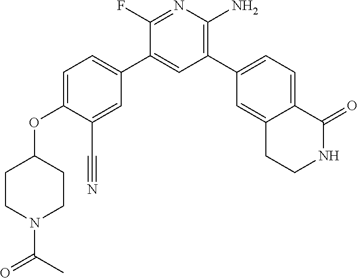 [SMILES: CC(=O)N1CCC(Oc2ccc(-c3cc(-c4ccc5c(c4)CCNC5=O)c(N)nc3F)cc2C#N)CC1]

A procedure analogous to Example 191 using 5-(6-amino-2-fluoro-5-(1-oxo-1,2,3,4-tetrahydroisoquinolin-6-yl)pyridin-3-yl)-2-(piperidin-4-yloxy)benzonitrile (27.1 mg, 0.059 mmol) and N,N-diisopropylethylamine (0.052 mL, 0.296 mmol) in CH₂Cl₂ (3 mL) and acetic anhydride (5.60 μl, 0.059 mmol) afforded the title compound (29.4 mg, 94%) as a white powder. ¹H NMR (500 MHz, DMSO-d₆) δ 7.95 (d, J=2.0 Hz, 2H), 7.91 (d, J=7.9 Hz, 1H), 7.83 (br d, J=8.9 Hz, 1H), 7.71 (d, J=10.3 Hz, 1H), 7.48 (dd, J=8.1, 1.5 Hz, 1H), 7.46 (s, 1H), 7.41 (d, J=9.0 Hz, 1H), 6.38 (s, 2H), 4.95-4.85 (m, 1H), 3.73-3.60 (m, 2H), 3.46-3.36 (m, 4H), 2.99-2.91 (m, 2H), 2.03 (s, 3H), 2.01-1.94 (m, 1H), 1.93-1.85 (m, 1H), 1.78-1.68 (m, 1H), 1.65-1.56 (m, 1H); LCMS: [M+H]⁺=500.46.

### Example 195: 6-(2-amino-6-fluoro-5-(1′-(2,2,2-trifluoroethyl)-4H-spiro[benzo[d][1,3]dioxine-2,4′-piperidin]-6-yl)pyridin-3-yl)-3,4-dihydroisoquinolin-1(2H)-one (I-195)

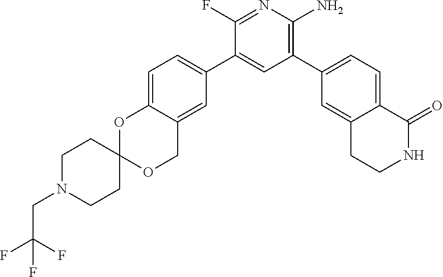 [SMILES: Nc1nc(F)c(-c2ccc3c(c2)COC2(CCN(CC(F)(F)F)CC2)O3)cc1-c1ccc2c(c1)CCNC2=O]

A procedure analogous to Example 186 using 6-(2-amino-6-fluoro-5-(4H-spiro[benzo[d][1,3]dioxine-2,4′-piperidin]-6-yl)pyridin-3-yl)-3,4-dihydroisoquinolin-1(2H)-one (21.8 mg, 0.047 mmol), N,N-diisopropylethylamine (0.082 mL, 0.473 mmol) in DMF (1 mL) and 2,2,2-trifluoroethyl trifluoromethanesulfonate (21.97 mg, 0.095 mmol) afforded the title compound (27.2 mg, 101% yield) as a white powder. ¹H NMR (500 MHz, DMSO-d₆) δ 7.94 (br s, 1H), 7.90 (d, J=7.9 Hz, 1H), 7.60 (d, J=10.1 Hz, 1H), 7.46 (d, J=7.9 Hz, 1H), 7.44 (s, 1H), 7.36 (br d, J=8.6 Hz, 1H), 7.27 (s, 1H), 6.88 (d, J=8.6 Hz, 1H), 6.24 (s, 2H), 4.89-4.82 (m, 2H), 3.44-3.37 (m, 2H), 3.25 (q, J=10.4 Hz, 2H), 2.95 (s, 2H), 2.73 (br s, 4H), 1.91-1.82 (m, 4H); LCMS: [M+H]⁺=543.50.

### Example 196: 6-(6-amino-2-fluoro-5-(1-oxo-1,2,3,4-tetrahydroisoquinolin-6-yl)pyridin-3-yl)-1′-(2,2,2-trifluoroethyl)spiro[chromane-2,4′-piperidin]-4-one (I-196)

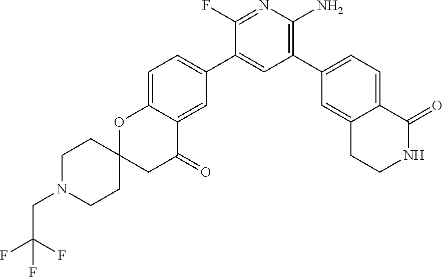 [SMILES: Nc1nc(F)c(-c2ccc3c(c2)C(=O)CC2(CCN(CC(F)(F)F)CC2)O3)cc1-c1ccc2c(c1)CCNC2=O]

A procedure similar to Example 186 using 6-(6-amino-2-fluoro-5-(1-oxo-1,2,3,4-tetrahydroisoquinolin-6-yl)pyridin-3-yl)spiro[chromane-2,4′-piperidin]-4-one (26.1 mg, 0.055 mmol) and N,N-diisopropylethylamine (0.096 mL, 0.552 mmol) in DMF (1 mL) and 2,2,2-trifluoroethyl trifluoromethanesulfonate (25.6 mg, 0.110 mmol) afforded the title compound (17.4 mg, 54%) as a white powder. ¹H NMR (500 MHz, DMSO-d₆) δ 7.94 (br s, 1H), 7.90 (d, J=7.9 Hz, 1H), 7.86 (s, 1H), 7.80-7.76 (m, 1H), 7.64 (d, J=10.3 Hz, 1H), 7.50-7.43 (m, 2H), 7.11 (d, J=8.6 Hz, 1H), 6.33 (s, 2H), 3.40 (dt, J=2.7, 6.5 Hz, 2H), 3.20 (q, J =10.3 Hz, 2H), 2.95 (t, J=6.5 Hz, 2H), 2.84 (s, 2H), 2.77-2.67 (m, 4H), 1.91 (br d, J=13.0 Hz, 2H), 1.80-1.70 (m, 2H); LCMS: [M+H]⁺=555.53.

### Example 197: 6-(2-amino-6-fluoro-5-(1′-(2,2,2-trifluoroethyl)spiro[chromane-2,4′-piperidin]-6-yl)pyridin-3-yl)-3,4-dihydroisoquinolin-1(2H)-one (I-197)

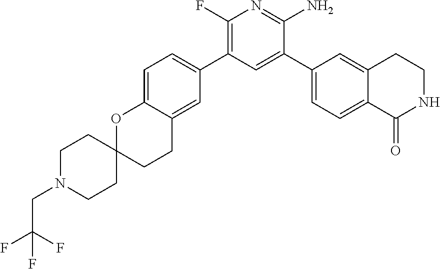 [SMILES: Nc1nc(F)c(-c2ccc3c(c2)CCC2(CCN(CC(F)(F)F)CC2)O3)cc1-c1ccc2c(c1)CCNC2=O]

A procedure similar to Example 186 using 6-(2-amino-6-fluoro-5-(spiro[chromane-2,4′-piperidin]-6-yl)pyridin-3-yl)-3,4-dihydroisoquinolin-1(2H)-one (23.3 mg, 0.051 mmol), N,N-diisopropylethylamine (0.089 mL, 0.508 mmol) in DMF (1 mL) and 2,2,2-trifluoroethyl trifluoromethanesulfonate (23.59 mg, 0.102 mmol) afforded the title compound (24.0 mg, 83%) as a white powder. ¹H NMR (500 MHz, DMSO-d₆) δ 7.94 (br s, 1H), 7.90 (d, J=7.9 Hz, 1H), 7.58 (d, J=10.1 Hz, 1H), 7.49-7.44 (m, 1H), 7.44 (s, 1H), 7.26 (s, 2H), 6.80 (d, J=8.2 Hz, 1H), 6.20 (s, 2H), 3.44-3.37 (m, 2H), 3.19 (q, J=10.2 Hz, 2H), 2.95 (s, 2H), 2.79-2.68 (m, 6H), 1.78 (s, 2H), 1.74-1.59 (m, 4H); LCMS: [M+H]⁺=541.46.

### Example 198: 5-(6-amino-2-fluoro-5-(1-oxo-1,2,3,4-tetrahydroisoquinolin-6-yl)pyridin-3-yl)-2-((1-(2,2,2-trifluoroethyl)piperidin-4-yl)oxy)benzonitrile (I-198)

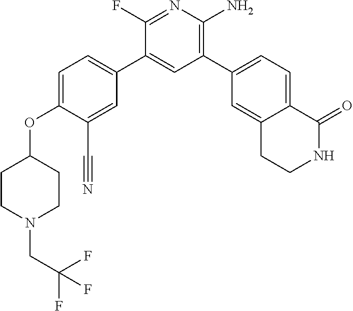 [SMILES: N#Cc1cc(-c2cc(-c3ccc4c(c3)CCNC4=O)c(N)nc2F)ccc1OC1CCN(CC(F)(F)F)CC1]

A procedure similar to Example 186 using 5-(6-amino-2-fluoro-5-(1-oxo-1,2,3,4-tetrahydroisoquinolin-6-yl)pyridin-3-yl)-2-(piperidin-4-yloxy)benzonitrile (22.4 mg, 0.049 mmol), N,N-diisopropylethylamine (0.085 mL, 0.490 mmol) in DMF (1 mL) and 2,2,2-trifluoroethyl trifluoromethanesulfonate (22.73 mg, 0.098 mmol) afforded the title compound (27.0 mg, 97%) as a white powder. ¹H NMR (500 MHz, DMSO-d₆) δ 7.98-7.88 (m, 3H), 7.81 (br d, J=9.0 Hz, 1H), 7.71 (d, J=10.3 Hz, 1H), 7.51-7.47 (m, 1H), 7.45 (s, 1H), 7.36 (d, J=9.2 Hz, 1H), 6.37 (s, 2H), 4.74-4.66 (m, 1H), 3.44-3.38 (m, 2H), 3.21 (q, J=10.1 Hz, 2H), 2.98-2.93 (m, 2H), 2.90-2.83 (m, 2H), 2.68-2.60 (m, 2H), 2.01-1.91 (m, 2H), 1.77-1.68 (m, 2H); LCMS: [M+H]⁺=540.49.

### Example 199: 6-(5-(4-((1-acetylpiperidin-4-yl)oxy)-3-(trifluoromethyl)phenyl)-2-amino-6-fluoropyridin-3-yl)-3,4-dihydroisoquinolin-1(2H)-one (I-199)

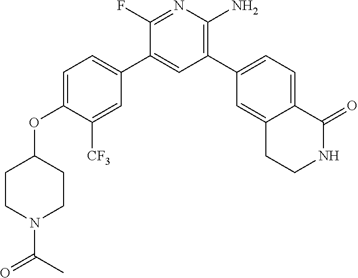 [SMILES: CC(=O)N1CCC(Oc2ccc(-c3cc(-c4ccc5c(c4)CCNC5=O)c(N)nc3F)cc2C(F)(F)F)CC1]

A procedure similar to Example 191 using 6-(2-amino-6-fluoro-5-(4-(piperidin-4-yloxy)-3-(trifluoromethyl)phenyl)pyridin-3-yl)-3,4-dihydroisoquinolin-1(2H)-one (23.0 mg, 0.046 mmol), N,N-diisopropylethylamine (0.040 ml, 0.230 mmol) in CH₂Cl₂ (3 mL) and acetic anhydride (4.34 μl, 0.046 mmol) afforded the title compound (25.8 mg, 98%) as a white powder. ¹H NMR (500 MHz, DMSO-d₆) δ 7.94 (br s, 1H), 7.91 (d, J=7.9 Hz, 1H), 7.79 (br d, J=8.8 Hz, 1H), 7.75 (s, 1H), 7.69 (d, J=10.1 Hz, 1H), 7.50-7.46 (m, 1H), 7.45 (s, 1H), 7.40 (d, J=8.9 Hz, 1H), 6.34 (s, 2H), 4.95-4.87 (m, 1H), 3.61-3.50 (m, 3H), 3.48-3.43 (m, 1H), 3.42-3.38 (m, 2H), 2.95 (s, 2H), 2.02 (s, 3H), 2.00-1.92 (m, 1H), 1.90-1.82 (m, 1H), 1.76-1.68 (m, 1H), 1.65-1.57 (m, 1H); LCMS: [M+H]⁺=543.57.

### Example 200: 6-(5-(4-(4-acetylpiperazin-1-yl)-3-(trifluoromethyl)phenyl)-2-amino-6-fluoropyridin-3-yl)-3,4-dihydroisoquinolin-1(2H)-one (I-200)

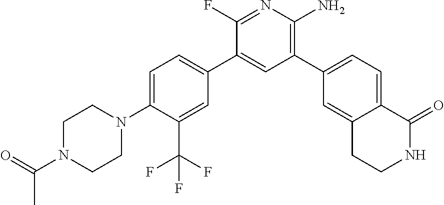 [SMILES: CC(=O)N1CCN(c2ccc(-c3cc(-c4ccc5c(c4)CCNC5=O)c(N)nc3F)cc2C(F)(F)F)CC1]

A procedure similar to Example 191 using 6-(2-amino-6-fluoro-5-(4-(piperazin-1-yl)-3-(trifluoromethyl)phenyl)pyridin-3-yl)-3,4-dihydroisoquinolin-1(2H)-one (20.8 mg, 0.043 mmol), N,N-diisopropylethylamine (0.037 mL, 0.214 mmol) in CH₂Cl₂ (3 mL) and acetic anhydride (4.05 μl, 0.043 mmol) afforded the title compound (22.8 mg, 96%) as a beige powder. ¹H NMR (500 MHz, DMSO-d₆) δ 7.95 (br s, 1H), 7.91 (d, J=7.9 Hz, 1H), 7.84 (br d, J=8.4 Hz, 1H), 7.81 (s, 1H), 7.73 (d, J=10.1 Hz, 1H), 7.60 (d, J=8.3 Hz, 1H), 7.48 (br d, J=8.1 Hz, 1H), 7.45 (s, 1H), 6.42 (s, 2H), 3.56 (br d, J=3.8 Hz, 4H), 3.44-3.37 (m, 2H), 2.95 (s, 2H), 2.89 (br s, 2H), 2.83 (br s, 2H), 2.04 (s, 3H); LCMS: [M+H]⁺=528.52

### Example 201: 6-(2-amino-6-fluoro-5-(4-((1-(2,2,2-trifluoroethyl)piperidin-4-yl)oxy)-3-(trifluoromethyl)phenyl)pyridin-3-yl)-3,4-dihydroisoquinolin-1(2H)-one (I-201)

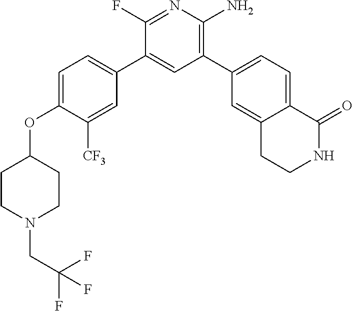 [SMILES: Nc1nc(F)c(-c2ccc(OC3CCN(CC(F)(F)F)CC3)c(C(F)(F)F)c2)cc1-c1ccc2c(c1)CCNC2=O]

A procedure similar to Example 186 using 6-(2-amino-6-fluoro-5-(4-(piperidin-4-yloxy)-3-(trifluoromethyl)phenyl)pyridin-3-yl)-3,4-dihydroisoquinolin-1(2H)-one (22.1 mg, 0.044 mmol), N,N-diisopropylethylamine (0.077 ml, 0.442 mmol) in DMF (1 mL) and 2,2,2-trifluoroethyl trifluoromethanesulfonate (20.50 mg, 0.088 mmol) afforded the title compound (19.6 mg, 72%) as a white powder. ¹H NMR (500 MHz, DMSO-d₆) δ 7.94 (br s, 1H), 7.90 (d, J=7.9 Hz, 1H), 7.76 (br d, J=8.9 Hz, 1H), 7.74 (s, 1H), 7.68 (d, J=10.3 Hz, 1H), 7.47 (dd, J=1.4, 8.0 Hz, 1H), 7.45 (s, 1H), 7.36 (d, J=8.8 Hz, 1H), 6.34 (s, 2H), 4.75-4.67 (m, 1H), 3.45-3.37 (m, 2H), 3.19 (q, J=10.2 Hz, 2H), 2.95 (t, J=6.5 Hz, 2H), 2.87-2.77 (m, 2H), 2.63 (dt, J=3.7, 7.5 Hz, 2H), 1.98-1.88 (m, 2H), 1.77-1.67 (m, 2H); LCMS: [M+H]⁺=583.52.

### Example 202: 6-(2-amino-6-fluoro-5-(4-(4-(2,2,2-trifluoroethyl)piperazin-1-yl)-3-(trifluoromethyl)phenyl)pyridin-3-yl)-3,4-dihydroisoquinolin-1(2H)-one (I-202)

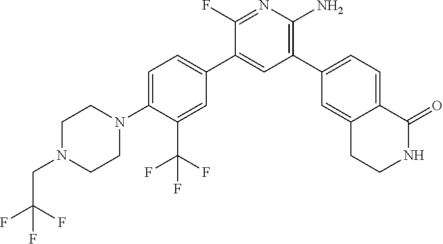 [SMILES: Nc1nc(F)c(-c2ccc(N3CCN(CC(F)(F)F)CC3)c(C(F)(F)F)c2)cc1-c1ccc2c(c1)CCNC2=O]

A procedure analogous to Example 186 using 6-(2-amino-6-fluoro-5-(4-(piperazin-1-yl)-3-(trifluoromethyl)phenyl)pyridin-3-yl)-3,4-dihydroisoquinolin-1(2H)-one (20.1 mg, 0.041 mmol), N,N-diisopropylethylamine (0.072 mL, 0.414 mmol) in DMF (1 mL) and 2,2,2-trifluoroethyl trifluoromethanesulfonate (19.22 mg, 0.083 mmol) afforded the title compound (15.7 mg, 64%) as a white powder. ¹H NMR (500 MHz, DMSO-d₆) δ 7.95 (br s, 1H), 7.90 (d, J=7.9 Hz, 1H), 7.82 (br d, J=8.7 Hz, 1H), 7.79 (s, 1H), 7.72 (d, J=10.1 Hz, 1H), 7.63 (d, J=8.4 Hz, 1H), 7.48 (dd, J=1.6, 7.9 Hz, 1H), 7.45 (s, 1H), 6.41 (s, 2H), 3.44-3.38 (m, 2H), 3.25 (q, J=10.2 Hz, 2H), 2.95 (br t, J=6.5 Hz, 2H), 2.92-2.87 (m, 4H), 2.77 (br s, 4H); LCMS: [M+H]⁺=568.55.

### Example 203: 6-(2-amino-5-(4-((1-(cyclopropanecarbonyl)piperidin-4-yl)oxy)phenyl)-6-fluoropyridin-3-yl)-3,4-dihydroisoquinolin-1(2H)-one (I-203)

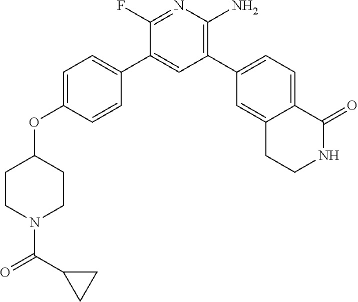 [SMILES: Nc1nc(F)c(-c2ccc(OC3CCN(C(=O)C4CC4)CC3)cc2)cc1-c1ccc2c(c1)CCNC2=O]

A procedure analogous to Example 191, using 6-(2-amino-6-fluoro-5-(4-(piperidin-4-yloxy)phenyl)pyridin-3-yl)-3,4-dihydroisoquinolin-1(2H)-one (23.7 mg, 0.055 mmol) and cyclopropanecarbonyl chloride (5.02 μl, 0.055 mmol) in CH₂Cl₂ (2 mL) and N,N-diisopropylethylamine (0.095 mL, 0.548 mmol), afforded the title compound (22.0 mg, 76%) as a white powder. ¹H NMR (500 MHz, DMSO-d₆) δ 7.97-7.92 (m, 1H), 7.90 (d, J=7.9 Hz, 1H), 7.60 (d, J=10.3 Hz, 1H), 7.47 (br d, J=8.3 Hz, 4H), 7.04 (d, J=8.8 Hz, 2H), 6.23 (s, 2H), 4.71-4.63 (m, 1H), 4.04-3.92 (m, 1H), 3.87 (br d, J=5.1 Hz, 1H), 3.54 (br s, 1H), 3.40 (dt, J=6.5, 2.7 Hz, 2H), 3.29-3.20 (m, 1H), 2.95 (t, J=6.5 Hz, 2H), 2.08-1.96 (m, 2H), 1.96-1.85 (m, 1H), 1.63 (br d, J=2.1 Hz, 1H), 1.52 (br s, 1H), 0.77-0.65 (m, 4H); LCMS: [M+H]⁺=501.51.

### Example 204: 6-(2-amino-5-(3,4-dihydro-2H-benzo[b][1,4]dioxepin-7-yl)-6-fluoropyridin-3-yl)-3,4-dihydroisoquinolin-1(2H)-one (I-204)

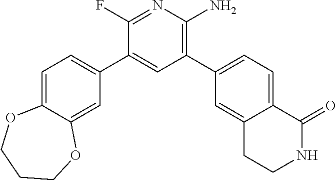 [SMILES: Nc1nc(F)c(-c2ccc3c(c2)OCCCO3)cc1-c1ccc2c(c1)CCNC2=O]

Step 1: 3-chloro-5-(3,4-dihydro-2H-benzo[b][1,4]dioxepin-7-yl)-6-fluoropyridin-2-amine

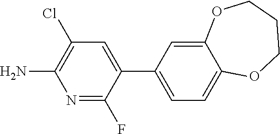 [SMILES: Nc1nc(F)c(-c2ccc3c(c2)OCCCO3)cc1Cl]

A procedure analogous to Example 177, Step 1 using 3,4-dihydro-2H-1,5-benzodioxepin-7-ylboronic acid (177 mg, 0.910 mmol), 5-bromo-3-chloro-6-fluoropyridin-2-amine (205.2 mg, 0.910 mmol), 1,4-dioxane (4 mL) and Cs₂CO₃ (890 mg, 2.73 mmol) in water (1 mL) and [1,12-bis(diphenylphosphino)ferrocene]dichloropalladium(II) (100 mg, 0.137 mmol) afforded the product (178.1 mg, 66%) as a white powder. LCMS: [M+H]⁺=295.19.

Step 2: 6-(2-amino-5-(3,4-dihydro-2H-benzo[b][1,4]dioxepin-7-yl)-6-fluoropyridin-3-yl)-3,4-dihydroisoquinolin-1(2H)-one

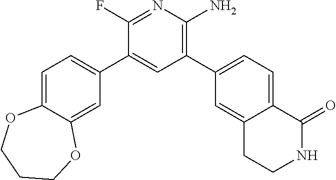 [SMILES: Nc1nc(F)c(-c2ccc3c(c2)OCCCO3)cc1-c1ccc2c(c1)CCNC2=O]

A procedure analogous to Example 43, Step 5 using 3-chloro-5-(3,4-dihydro-2H-benzo[b][1,4]dioxepin-7-yl)-6-fluoropyridin-2-amine (52 mg, 0.176 mmol), 3,4-dihydro-1(2H)-isoquinolinone-6-boronic acid pinacol ester (53.0 mg, 0.194 mmol), 1,4-dioxane (3 mL), K₃PO₄ (112 mg, 0.529 mmol) in water (1 mL) and XPhos Pd G2 (13.88 mg, 0.018 mmol) afforded the title compound (31.0 mg, 41%) as a white powder. ¹H NMR (500 MHz, DMSO-d₆) δ 7.94 (br s, 1H), 7.90 (d, J=7.9 Hz, 1H), 7.60 (d, J=10.1 Hz, 1H), 7.50-7.43 (m, 2H), 7.16 (s, 1H), 7.13 (br d, J=8.4 Hz, 1H), 6.99 (d, J=8.3 Hz, 1H), 6.28 (s, 2H), 4.18-4.11 (m, 4H), 3.43-3.38 (m, 2H), 2.98-2.92 (m, 2H), 2.15-2.07 (m, 2H); LCMS: [M+H]⁺=406.41.

### Example 205: 6-(2-amino-5-(3,4-dihydro-2H-benzo[b][1,4]dioxepin-7-yl)-6-fluoropyridin-3-yl)-7-fluoro-3,4-dihydroisoquinolin-1(2H)-one (I-205)

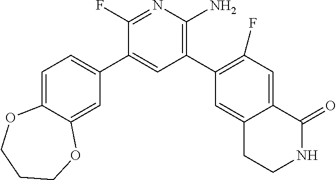 [SMILES: Nc1nc(F)c(-c2ccc3c(c2)OCCCO3)cc1-c1cc2c(cc1F)C(=O)NCC2]

A procedure analogous to Example 43, Step 5 using 3-chloro-5-(3,4-dihydro-2H-benzo[b][1,4]dioxepin-7-yl)-6-fluoropyridin-2-amine (51.6 mg, 0.175 mmol), 7-fluoro-6-(4,4,5,5-tetramethyl-1,3,2-dioxaborolan-2-yl)-3,4-dihydroisoquinolin-1(2H)-one (56.1 mg, 0.193 mmol), 1,4-dioxane (3 mL), K₃PO₄ (111 mg, 0.525 mmol) in water (1 mL) and XPhos Pd G2 (13.78 mg, 0.018 mmol) afforded the title compound (30.5 mg, 39%) as a beige powder. ¹H NMR (500 MHz, DMSO-d₆) δ 8.10 (brs, 1H), 7.60 (dd, J=10.1, 3.0 Hz, 2H), 7.43 (d, J=7.1 Hz, 1H), 7.16-7.07 (m, 2H), 6.99 (d, J=8.3 Hz, 1H), 6.28 (s, 2H), 4.18-4.10 (m, 4H), 3.44-3.38 (m, 2H), 2.95-2.89 (m, 2H), 2.15-2.07 (m, 2H); LCMS: [M+H]⁺=424.39.

### Example 206: 6-(2-amino-6-fluoro-5-(4-((4-methyl-1,4-diazepan-1-yl)sulfonyl)phenyl)pyridin-3-yl)-3,4-dihydroisoquinolin-1(2H)-one (I-206)

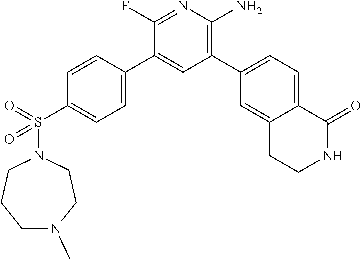 [SMILES: CN1CCCN(S(=O)(=O)c2ccc(-c3cc(-c4ccc5c(c4)CCNC5=O)c(N)nc3F)cc2)CC1]

A procedure analogous to Example 43, Step 5 using 6-(2-amino-5-bromo-6-fluoropyridin-3-yl)-3,4-dihydroisoquinolin-1(2H)-one (100 mg, 0.261 mmol), 1-[(4-bromobenzene)sulfonyl]-4-methylhomopiperazine (109 mg, 0.326 mmol), [1,12-bis(diphenylphosphino)ferrocene]dichloropalladium(II) (15.91 mg, 0.022 mmol), K₃PO₄ (0.335 mL of a 1.3 M aqueous solution, 0.435 mmol), DME (4 mL) and H₂O (2 mL) afforded the title compound (50 mg, 48%) as a tan solid. ¹H NMR (500 MHz, DMSO-d₆) δ 7.95 (br s, 1H), 7.91 (d, J=7.9 Hz, 1H), 7.82-7.77 (m, 4H), 7.75 (d, J=10.1 Hz, 1H), 7.48 (d, J=7.9 Hz, 1H), 7.46 (s, 1H), 6.52 (s, 2H), 3.42-3.39 (m, 3H), 3.31-3.27 (m, 4H), 2.55-2.52 (m, 2H), 2.49-2.45 (m, 2H), 2.22 (s, 4H), 1.73 (quin, J=5.7 Hz, 2H); LCMS: [M+1]⁺=510.39.

### Example 207: 6-(2-amino-6-fluoro-5-(4-((4-methylpiperazin-1-yl)sulfonyl)phenyl)pyridin-3-yl)-3,4-dihydroisoquinolin-1(2H)-one (I-207)

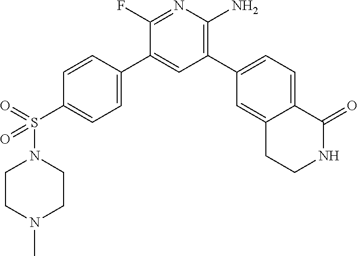 [SMILES: CN1CCN(S(=O)(=O)c2ccc(-c3cc(-c4ccc5c(c4)CCNC5=O)c(N)nc3F)cc2)CC1]

A procedure analogous to Example 43, Step 5 using 6-(2-amino-5-bromo-6-fluoropyridin-3-yl)-3,4-dihydroisoquinolin-1(2H)-one (115 mg, 0.300 mmol), 1-(4-bromophenylsulfonyl)-4-methylpiperazine (120 mg, 0.375 mmol), [1,12-bis(diphenylphosphino)ferrocene]dichloropalladium(II) (18.30 mg, 0.025 mmol), aqueous K₃PO₄ (0.385 mL of a 1.3 M solution, 0.500 mmol), DME (4 mL) and H₂O (2 mL) afforded the title compound (0.108 mmol, 43%) as a tan solid. ¹H NMR (500 MHz, DMSO-d₆) δ 7.97-7.93 (m, 1H), 7.93-7.89 (m, 1H), 7.87-7.83 (m, 2H), 7.79-7.71 (m, 3H), 7.51-7.44 (m, 2H), 6.54 (br s, 2H), 3.40 (br d, J=6.8 Hz, 2H), 2.97-2.93 (m, 2H), 2.90 (br s, 4H), 2.36 (br s, 4H), 2.13 (s, 3H); LCMS: [M+1]⁺=496.47.

### Example 208: 6-(2-amino-6-fluoro-5-(4-(4-methoxy-1-(2-methoxyethyl)piperidin-4-yl)phenyl)pyridin-3-yl)-3,4-dihydroisoquinolin-1(2H)-one (I-208)

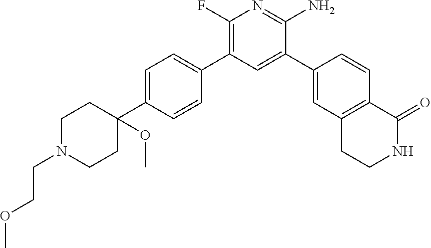 [SMILES: COCCN1CCC(OC)(c2ccc(-c3cc(-c4ccc5c(c4)CCNC5=O)c(N)nc3F)cc2)CC1]

Step 1: tert-butyl 4-(4-(6-amino-2-fluoro-5-(1-oxo-1,2,3,4-tetrahydroisoquinolin-6-yl)pyridin-3-yl)phenyl)-4-methoxypiperidine-1-carboxylate

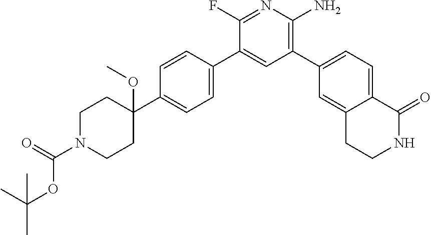 [SMILES: COC1(c2ccc(-c3cc(-c4ccc5c(c4)CCNC5=O)c(N)nc3F)cc2)CCN(C(=O)OC(C)(C)C)CC1]

A procedure analogous to that of Example 43, Step 1 heating tert-butyl 4-(4-chlorophenyl)-4-methoxypiperidine-1-carboxylate (59.5 mg, 0.164 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (88 mg, 0.230 mmol) at 85° C. for 1.75 h afforded the title compound as a white powder (55 mg, 56% yield based on purity of 92%). LCMS: [M+H]⁺=547.70.

Step 2: 6-(2-amino-6-fluoro-5-(4-(4-methoxy-1-(2-methoxyethyl)piperidin-4-yl)phenyl)pyridin-3-yl)-3,4-dihydroisoquinolin-1(2H)-one

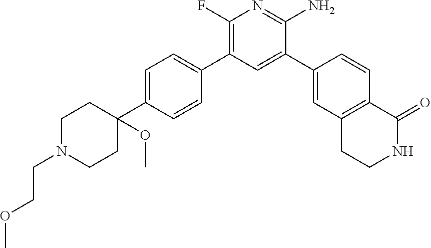 [SMILES: COCCN1CCC(OC)(c2ccc(-c3cc(-c4ccc5c(c4)CCNC5=O)c(N)nc3F)cc2)CC1]

A solution of 4.0 M HCl in dioxane (0.629 mL, 2.52 mmol) was added to a solution of tert-butyl 4-(4-(6-amino-2-fluoro-5-(1-oxo-1,2,3,4-tetrahydroisoquinolin-6-yl)pyridin-3-yl)phenyl)-4-methoxypiperidine-1-carboxylate (55 mg, 0.101 mmol) in MeOH (4 mL). The reaction mixture was stirred at RT for 2.5 h and concentrated onto Celite®. Reverse phase chromatography (C18; 0-20% acetonitrile/water) followed by filtration through a Waters PoraPak CX column afforded 56 mg of the intermediate 6-(2-amino-6-fluoro-5-(4-(4-methoxypiperidin-4-yl)phenyl)pyridin-3-yl)-3,4-dihydroisoquinolin-1(2H)-one as a beige solid. This intermediate was taken up in DMF (3 mL) with 2-bromoethyl methyl ether (0.047 mL, 0.503 mmol) and Et₃N (0.210 ml, 1.51 mmol) and stirred overnight at RT. The reaction mixture was concentrated onto Celite® and purified by reverse phase chromatography (C18; 0-40% acetonitrile/water) to afford the title compound as a beige powder (27 mg, 51%). ¹H NMR (500 MHz, DMSO-d₆) δ 7.99-7.94 (m, 1H), 7.91 (d, J=7.9 Hz, 1H), 7.71-7.66 (m, 1H), 7.58 (br d, J=7.6 Hz, 2H), 7.51-7.43 (m, 3H), 6.38-6.30 (m, 2H), 3.56-3.51 (m, 4H), 3.44-3.41 (m, 5H), 3.28 (br s, 3H), 2.99-2.89 (m, 5H), 2.89-2.89 (m, 1H), 2.19-1.77 (m, 4H); LCMS: [M+H]⁺=505.65.

### Example 209: 6-(2-amino-6-chloro-5-(4-(4-isopropylpiperazin-1-yl)phenyl)pyridin-3-yl)-7-fluoro-3,4-dihydroisoquinolin-1(2H)-one (I-209)

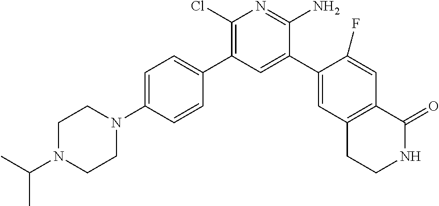 [SMILES: CC(C)N1CCN(c2ccc(-c3cc(-c4cc5c(cc4F)C(=O)NCC5)c(N)nc3Cl)cc2)CC1]

Step 1: 6-(2-amino-5-bromo-6-chloropyridin-3-yl)-7-fluoro-3,4-dihydroisoquinolin-1(2H)-one

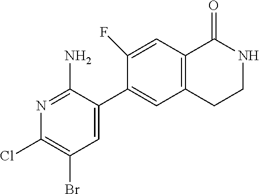 [SMILES: Nc1nc(Cl)c(Br)cc1-c1cc2c(cc1F)C(=O)NCC2]

A mixture of 5-bromo-6-chloro-3-iodopyridin-2-amine (500 mg, 1.50 mmol), 7-fluoro-6-(4,4,5,5-tetramethyl-1,3,2-dioxaborolan-2-yl)-3,4-dihydroisoquinolin-1(2H)-one (570 mg, 1.70 mmol, 87% purity) Na₂CO₃—H₂O (391 mg, 3.15 mmol) and bis(triphenylphosphine)palladium(II) dichloride (126 mg, 0.180 mmol), in CH₃CN (28 mL) and H₂O (12 mL) was heated in a microwave apparatus at 80° C. for 4 h. The reaction mixture was partitioned between brine and EtOAc. The organic phase was separated and aqueous phase was further extracted with DCM (2×10 mL). The combined organic layers were combined, dried over Na₂SO₄ and concentrated onto Celite®. Silica gel chromatography (eluting with 0-100% EtOAc/Hexanes) afforded the product as a beige solid (314 mg, 57%); LCMS: [M+H]⁺=372.33.

Step 2: 6-(2-Amino-6-chloro-5-(4-(4-isopropylpiperazin-1-yl)phenyl)pyridin-3-yl)-7-fluoro-3,4-dihydroisoquinolin-1(2H)-one

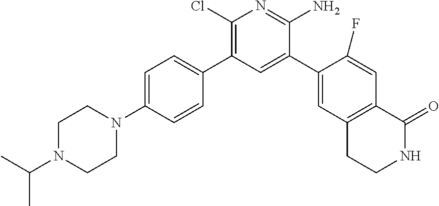 [SMILES: CC(C)N1CCN(c2ccc(-c3cc(-c4cc5c(cc4F)C(=O)NCC5)c(N)nc3Cl)cc2)CC1]

A procedure analogous to that of Example 45, step 5 using 6-(2-amino-5-bromo-6-chloropyridin-3-yl)-7-fluoro-3,4-dihydroisoquinolin-1(2H)-one (50 mg, 0.135 mmol) and 4-(4-isopropylpiperazinyl)phenylboronic acid, pinacol ester (62.4 mg, 0.189 mmol) at 90° C. for 1.75 h afforded the title compound as a beige powder (30 mg, 43%). ¹H NMR (500 MHz, DMSO-d₆) δ 8.20-8.05 (m, 1H), 7.68-7.56 (m, 1H), 7.48-7.39 (m, 1H), 7.39-7.37 (m, 1H), 7.39-7.24 (m, 2H), 6.96 (br d, J=6.5 Hz, 2H), 6.35-6.15 (m, 2H), 3.49-3.38 (m, 2H), 3.21-3.05 (m, 4H), 2.98-2.88 (m, 2H), 2.76-2.66 (m, 1H), 2.64-2.54 (m, 4H), 1.11-0.97 (m, 6H). LCMS: [M+H]⁺=494.55.

### Example 210: 6-(2-amino-5-(4-(4-(cyclopropylmethyl)piperazin-1-yl)phenyl)-6-fluoropyridin-3-yl)-3,4-dihydroisoquinolin-1(2H)-one (I-210)

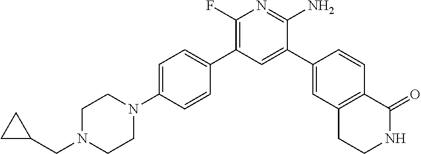 [SMILES: Nc1nc(F)c(-c2ccc(N3CCN(CC4CC4)CC3)cc2)cc1-c1ccc2c(c1)CCNC2=O]

To a solution of 6-(2-amino-6-fluoro-5-(4-(piperazin-1-yl)phenyl)pyridin-3-yl)-3,4-dihydroisoquinolin-1(2H)-one-TFA (40 mg, 0.075 mmol) and N,N-diisopropylethylamine (0.131 ml, 0.753 mmol) in DMF (1.0 mL) was added (bromomethyl)cyclopropane (15.24 mg, 0.113 mmol). The reaction was stirred at 70° C. for 4 h. The mixture was concentrated onto Celite® and purified by reverse phase chromatography (C18, 0-100% MeCN—H₂O) to give the title compound (13.6 mg, 0.029 mmol, 38%) as a beige solid. ¹H NMR (500 MHz, DMSO-d₆) δ 7.83 (br s, 1H), 7.79 (d, J=7.8 Hz, 1H), 7.47 (d, J=10.2 Hz, 1H), 7.36 (br d, J=8.1 Hz, 1H), 7.34 (s, 1H), 7.29 (br d, J=8.1 Hz, 2H), 6.87 (br d, J=8.7 Hz, 2H), 6.07 (s, 2H), 3.28-3.31 (m, 2H), 3.06 (br d, J=5.3 Hz, 4H), 2.84 (br t, J=6.4 Hz, 2H), 2.48 (br s, 2H), 2.13 (br s, 2H), 0.75 (br d, J=6.6 Hz, 1H), 0.38 (br d, J=6.9 Hz, 2H), 0.00 (br s, 2H); LCMS: [M+H]⁺=472.55.

### Example 211: 6-(2-amino-5-(4-(4-ethylpiperazin-1-yl)phenyl)-6-fluoropyridin-3-yl)-7-fluoro-3,4-dihydroisoquinolin-1(2H)-one (I-211)

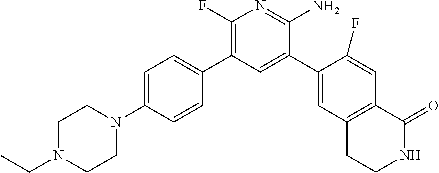 [SMILES: CCN1CCN(c2ccc(-c3cc(-c4cc5c(cc4F)C(=O)NCC5)c(N)nc3F)cc2)CC1]

To a suspension of 6-(2-amino-5-bromo-6-fluoropyridin-3-yl)-7-fluoro-3,4-dihydroisoquinolin-1(2H)-one (40 mg, 0.113 mmol), 1-ethyl-4-(4-(4,4,5,5-tetramethyl-1,3,2-dioxaborolan-2-yl)phenyl)piperazine (42.9 mg, 0.136 mmol) and Cs₂CO₃ (92 mg, 0.282 mmol) in a 3:1 mixture of DME (6 mL): water (2 mL) was added PdCl₂dppf (8.26 mg, 0.011 mmol). The mixture was flushed with nitrogen then heated in the microwave at 90° C. for 2 hours. The reaction was concentrated onto Celite® and purified by silica gel chromatography eluting with 0-10% MeOH/DCM, followed by reverse phase chromatography (C18, 0-100% ACN/H₂O) to afford the title compound (9.7 mg, 19%) as a beige solid. ¹H NMR (500 MHz, DMSO-d₆) δ 8.11 (br s, 1H), 7.60 (dd, J=13.0, 10.2 Hz, 2H), 7.43 (d, J=7.0 Hz, 1H), 7.38 (d, J=7.8 Hz, 2H), 6.97 (d, J=8.9 Hz, 2H), 6.19 (s, 2H), 3.41 (td, J=6.5, 2.8 Hz, 2H), 3.14-3.20 (m, 4H), 2.92 (br t, J=6.5 Hz, 2H), 2.37 (q, J=7.2 Hz, 2H), 1.04 (t, J=7.2 Hz, 3H); LCMS: [M+H]⁺=464.47.

### Example 212: 6-(2-amino-6-fluoro-5-(4-(4-(2-methoxyethyl)piperazin-1-yl)phenyl)pyridin-3-yl)-7-fluoro-3,4-dihydroisoquinolin-1(2H)-one (I-212)

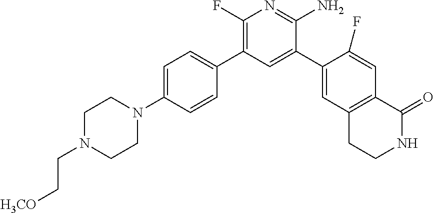 [SMILES: COCCN1CCN(c2ccc(-c3cc(-c4cc5c(cc4F)C(=O)NCC5)c(N)nc3F)cc2)CC1]

To a solution of 6-(2-amino-6-fluoro-5-(4-(piperazin-1-yl)phenyl)pyridin-3-yl)-7-fluoro-3,4-dihydroisoquinolin-1(2H)-one-TFA (40 mg, 0.073 mmol) and N,N-diisopropylethylamine (0.127 ml, 0.728 mmol) in DMF (1.0 mL) was added 2-bromoethyl methyl ether (15.18 mg, 0.109 mmol). The reaction was stirred at 70° C. for 2 h. The mixture was concentrated onto Celite® and purified by reverse phase chromatography (C18, 0-100% MeCN—H2O) to give the title compound (16 mg, 45%) as a beige solid. ¹H NMR (500 MHz, DMSO-d₆) δ 8.04 (br s, 1H), 7.52 (dd, J=12.5, 10.3 Hz, 2H), 7.35 (d, J=7.1 Hz, 1H), 7.31 (br d, J=8.0 Hz, 2H), 6.90 (br d, J=7.8 Hz, 2H), 6.12 (br s, 2H), 3.41 (br d, J=2.5 Hz, 2H), 3.33 (br dd, J=6.5, 2.5 Hz, 2H), 3.16-3.21 (m, 3H), 3.07 (br s, 4H), 2.85 (br t, J=6.4 Hz, 2H), 2.49 (br s, 2H); LCMS: [M+H]⁺=494.55.

### Example 213: 6-(2-amino-5-(4-(4-(cyclopropylmethyl)piperazin-1-yl)phenyl)-6-fluoropyridin-3-yl)-7-fluoro-3,4-dihydroisoquinolin-1(2H)-one (I-213)

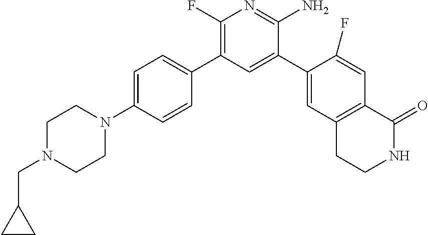 [SMILES: Nc1nc(F)c(-c2ccc(N3CCN(CC4CC4)CC3)cc2)cc1-c1cc2c(cc1F)C(=O)NCC2]

A procedure similar to Example 212 using 6-(2-amino-6-fluoro-5-(4-(piperazin-1-yl)phenyl)pyridin-3-yl)-7-fluoro-3,4-dihydroisoquinolin-1(2H)-one-TFA (25 mg, 0.045 mmol) and N,N-diisopropylethylamine (0.079 mL, 0.455 mmol) in DMF (1.0 mL) and (bromomethyl)cyclopropane (9.21 mg, 0.068 mmol) afforded the title compound (5.4 mg, 24%) as a beige solid. ¹H NMR (500 MHz, DMSO-d₆) δ 8.00 (br s, 1H), 7.49 (dd, J=12.8, 10.2 Hz, 2H), 7.32 (d, J=7.1 Hz, 1H), 7.28 (d, J=7.8 Hz, 2H), 6.87 (d, J=8.9 Hz, 2H), 6.08 (s, 2H), 3.29-3.31 (m, 2H), 3.04-3.09 (m, 4H), 2.82 (br t, J=6.4 Hz, 2H), 2.46-2.50 (m, 4H), 2.13 (br d, J=6.6 Hz, 2H), 0.73-0.78 (m, 1H), 0.35-0.41 (m, 2H),−0.03-0.02 (m, 2H); LCMS: [M+H]⁺=490.67.

### Example 214: 6-(2-amino-6-fluoro-5-(1′-(2-methoxyethyl)-4H-spiro[benzo[d][1,3]dioxine-2,4′-piperidin]-6-yl)pyridin-3-yl)-3,4-dihydroisoquinolin-1(2H)-one (I-214)

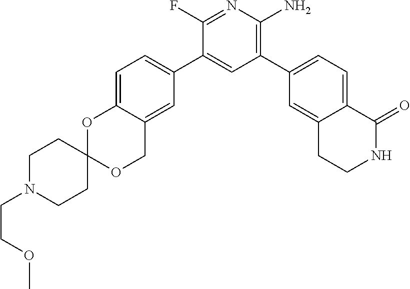 [SMILES: COCCN1CCC2(CC1)OCc1cc(-c3cc(-c4ccc5c(c4)CCNC5=O)c(N)nc3F)ccc1O2]

A procedure analogous to Example 186 using 6-(2-amino-6-fluoro-5-(4H-spiro[benzo[d][1,3]dioxine-2,4′-piperidin]-6-yl)pyridin-3-yl)-3,4-dihydroisoquinolin-1(2H)-one (29.2 mg, 0.063 mmol), N,N-diisopropylethylamine (0.110 mL, 0.634 mmol) DMF (1 mL) and 2-bromoethyl methyl ether (0.018 ml, 0.190 mmol) afforded the title compound (31.0 mg, 85%) as a beige powder. ¹H NMR (500 MHz, DMSO-d₆) δ 7.94 (br s, 1H), 7.90 (d, J=7.9 Hz, 1H), 7.60 (d, J=10.1 Hz, 1H), 7.49-7.42 (m, 2H), 7.35 (br d, J=8.6 Hz, 1H), 7.26 (s, 1H), 6.87 (d, J=8.6 Hz, 1H), 6.24 (s, 2H), 4.85 (s, 2H), 3.46-3.38 (m, 5H), 3.23 (s, 3H), 2.95 (br t, J=6.5 Hz, 2H), 2.52 (br s, 4H), 1.89-1.78 (m, 4H); LCMS: [M+H]⁺=519.57.

### Example 215: 6-(6-amino-2-fluoro-5-(1-oxo-1,2,3,4-tetrahydroisoquinolin-6-yl)pyridin-3-yl)-1′-(2-methoxyethyl)spiro[chromane-2,4′-piperidin]-4-one (I-215)

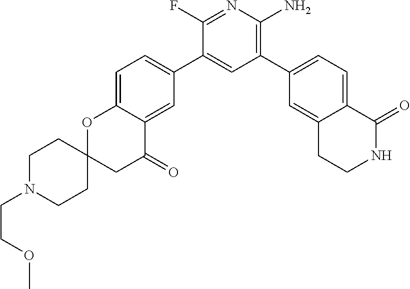 [SMILES: COCCN1CCC2(CC1)CC(=O)c1cc(-c3cc(-c4ccc5c(c4)CCNC5=O)c(N)nc3F)ccc1O2]

A procedure analogous to Example 186 using 6-(6-amino-2-fluoro-5-(1-oxo-1,2,3,4-tetrahydroisoquinolin-6-yl)pyridin-3-yl)spiro[chromane-2,4′-piperidin]-4-one (26.1 mg, 0.055 mmol) and N,N-diisopropylethylamine (0.096 ml, 0.552 mmol), DMF (1 mL) and 2-bromoethyl methyl ether (0.016 ml, 0.166 mmol) afforded the title compound (23.1 mg, 73%) as a slightly yellow powder. ¹H NMR (500 MHz, DMSO-d₆) δ 7.94 (br s, 1H), 7.90 (d, J=7.9 Hz, 1H), 7.85 (s, 1H), 7.80-7.75 (m, 1H), 7.64 (d, J=10.3 Hz, 1H), 7.50-7.46 (m, 1H), 7.45 (s, 1H), 7.10 (d, J=8.7 Hz, 1H), 6.33 (s, 2H), 3.46-3.38 (m, 5H), 3.22 (s, 3H), 2.95 (br t, J=6.5 Hz, 2H), 2.82 (s, 2H), 2.65-2.57 (m, 2H), 2.38 (br t, J=10.6 Hz, 2H), 1.94-1.85 (m, 2H), 1.76-1.66 (m, 2H); LCMS: [M+H]⁺=531.38.

### Example 216: 6-(2-amino-6-fluoro-5-(1′-(2-methoxyethyl)spiro[chromane-2,4′-piperidin]-6-yl)pyridin-3-yl)-3,4-dihydroisoquinolin-1(2H)-one (I-216)

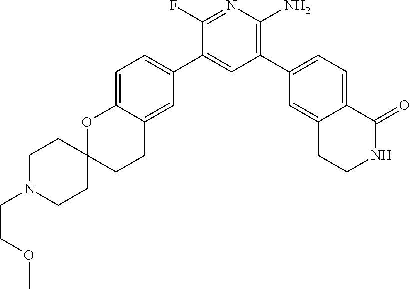 [SMILES: COCCN1CCC2(CCc3cc(-c4cc(-c5ccc6c(c5)CCNC6=O)c(N)nc4F)ccc3O2)CC1]

A procedure similar to Example 186, using 6-(2-amino-6-fluoro-5-(spiro[chromane-2,4′-piperidin]-6-yl)pyridin-3-yl)-3,4-dihydroisoquinolin-1(2H)-one (28.3 mg, 0.062 mmol), N,N-diisopropylethylamine (0.108 mL, 0.617 mmol), DMF (1 mL) and 2-bromoethyl methyl ether (0.017 mL, 0.185 mmol) afforded the title compound (30.0 mg, 88%) as a beige powder. ¹H NMR (500 MHz, DMSO-d₆) δ 7.94 (br s, 1H), 7.90 (d, J=7.9 Hz, 1H), 7.57 (d, J=10.1 Hz, 1H), 7.48-7.45 (m, 1H), 7.44 (s, 1H), 7.27-7.21 (m, 2H), 6.79 (d, J=8.3 Hz, 1H), 6.19 (s, 2H), 3.42 (br d, J=6.0 Hz, 5H), 3.23 (s, 3H), 2.95 (t, J=6.5 Hz, 2H), 2.74 (br t, J=6.8 Hz, 2H), 2.63-2.55 (m, 2H), 2.39 (br t, J=9.7 Hz, 2H), 1.77 (br t, J=6.7 Hz, 2H), 1.73-1.66 (m, 2H), 1.65-1.55 (m, 2H); LCMS: [M+H]⁺=517.31.n

### Example 217: 5-(6-amino-2-fluoro-5-(1-oxo-1,2,3,4-tetrahydroisoquinolin-6-yl)pyridin-3-yl)-2-((1-(2-methoxyethyl)piperidin-4-yl)oxy)benzonitrile (I-217)

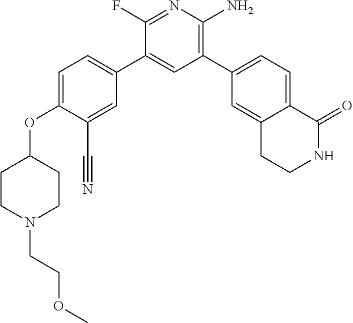 [SMILES: COCCN1CCC(Oc2ccc(-c3cc(-c4ccc5c(c4)CCNC5=O)c(N)nc3F)cc2C#N)CC1]

A procedure analogous to Example 186 using 5-(6-amino-2-fluoro-5-(1-oxo-1,2,3,4-tetrahydroisoquinolin-6-yl)pyridin-3-yl)-2-(piperidin-4-yloxy)benzonitrile (29.5 mg, 0.064 mmol) and N,N-diisopropylethylamine (0.112 mL, 0.645 mmol), DMF (1 mL) and 2-bromoethyl methyl ether (0.018 mL, 0.193 mmol) afforded the title compound (32.1 mg, 91%) as a beige powder. ¹H NMR (500 MHz, DMSO-d₆) δ 7.95 (br s, 1H), 7.92 (d, J=2.2 Hz, 1H), 7.90 (d, J=7.9 Hz, 1H), 7.81 (br d, J=9.2 Hz, 1H), 7.71 (d, J=10.3 Hz, 1H), 7.48 (dd, J=7.9, 1.5 Hz, 1H), 7.45 (s, 1H), 7.35 (d, J=9.2 Hz, 1H), 6.37 (s, 2H), 4.70-4.62 (m, 1H), 3.46-3.38 (m, 5H), 3.23 (s, 3H), 2.95 (t, J=6.5 Hz, 2H), 2.72-2.63 (m, 2H), 2.40-2.32 (m, 2H), 1.99-1.89 (m, 2H), 1.74-1.65 (m, 2H); LCMS: [M−H]⁻=514.53.

### Example 218: 6-(2-amino-6-fluoro-5-(1′-isopropyl-4H-spiro[benzo[d][1,3]dioxine-2,4′-piperidin]-6-yl)pyridin-3-yl)-3,4-dihydroisoquinolin-1(2H)-one (I-218)

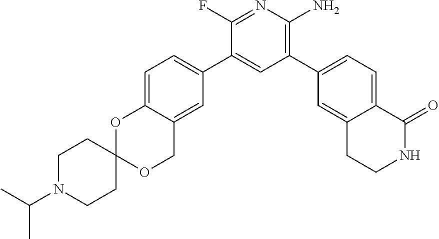 [SMILES: CC(C)N1CCC2(CC1)OCc1cc(-c3cc(-c4ccc5c(c4)CCNC5=O)c(N)nc3F)ccc1O2]

To a solution of 6-(2-amino-6-fluoro-5-(4H-spiro[benzo[d][1,3]dioxine-2,4′-piperidin]-6-yl)pyridin-3-yl)-3,4-dihydroisoquinolin-1(2H)-one (26.0 mg, 0.056 mmol) in CH₂Cl₂ (1.5 mL) and acetone (1.5 mL) was added 2 drops of glacial AcOH (24 mg, 0.400 mmol) at RT and the reaction mixture was stirred for 30 min. Then NaBH(OAc)₃ (50 mg, 0.236 mmol) was added and the reaction mixture was stirred at 23° C. for 16 h. The reaction mixture was dry loaded onto Celite® and purified by flash chromatography (0-20% DCM/MeOH) to afford the title compound (22.3 mg, 75%) as a white powder. ¹H NMR (500 MHz, DMSO-d₆) δ 7.94 (br s, 1H), 7.90 (d, J=7.9 Hz, 1H), 7.60 (d, J=10.1 Hz, 1H), 7.46 (d, J=8.1 Hz, 1H), 7.44 (s, 1H), 7.35 (br d, J=8.6 Hz, 1H), 7.26 (s, 1H), 6.87 (d, J=8.6 Hz, 1H), 6.24 (s, 2H), 4.84 (s, 2H), 3.41-3.38 (m, 4H), 2.99-2.92 (m, 2H), 2.74 (td, J=13.0, 6.4 Hz, 1H), 2.57-2.51 (m, 2H), 1.88-1.76 (m, 4H), 0.98 (d, J=6.5 Hz, 6H); LCMS: [M+H]⁺=503.55.

### Example 219: 6-(2-amino-6-fluoro-5-(4-(piperazin-1-yl)-3-(trifluoromethyl)phenyl)pyridin-3-yl)-7-fluoro-3,4-dihydroisoquinolin-1(2H)-one (I-219)

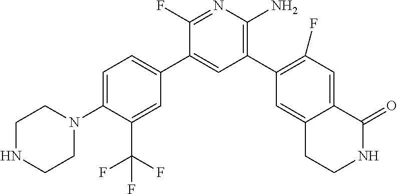 [SMILES: Nc1nc(F)c(-c2ccc(N3CCNCC3)c(C(F)(F)F)c2)cc1-c1cc2c(cc1F)C(=O)NCC2]

Step 1: tert-butyl 4-(4-(6-amino-2-fluoro-5-(7-fluoro-1-oxo-1,2,3,4-tetrahydroisoquinolin-6-yl)pyridin-3-yl)-2-(trifluoromethyl)phenyl)piperazine-1-carboxylate

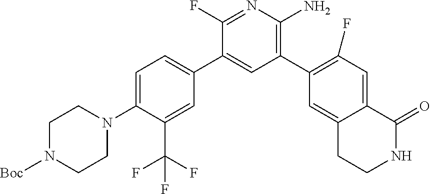 [SMILES: CC(C)(C)OC(=O)N1CCN(c2ccc(-c3cc(-c4cc5c(cc4F)C(=O)NCC5)c(N)nc3F)cc2C(F)(F)F)CC1]

A procedure similar to Example 43, Step 5 using tert-butyl 4-(4-(6-amino-5-chloro-2-fluoropyridin-3-yl)-2-(trifluoromethyl)phenyl)piperazine-1-carboxylate (152 mg, 0.320 mmol), 7-fluoro-6-(4,4,5,5-tetramethyl-1,3,2-dioxaborolan-2-yl)-3,4-dihydroisoquinolin-1(2H)-one (112 mg, 0.384 mmol), 1,4-dioxane (4 mL), K₃PO₄ (204 mg, 0.960 mmol) in water (1 mL) and XPhos Pd G2 (25.2 mg, 0.032 mmol) afforded the product (114.4 mg, 56%) as a dark beige powder which was used for the next step. LCMS: [M+H]⁺=604.51.

Step 2: 6-(2-amino-6-fluoro-5-(4-(piperazin-1-yl)-3-(trifluoromethyl)phenyl)pyridin-3-yl)-7-fluoro-3,4-dihydroisoquinolin-1(2H)-one

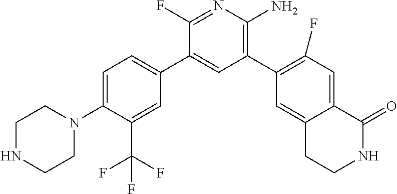 [SMILES: Nc1nc(F)c(-c2ccc(N3CCNCC3)c(C(F)(F)F)c2)cc1-c1cc2c(cc1F)C(=O)NCC2]

A procedure similar to Example 144, Step 3 using tert-butyl 4-(4-(6-amino-2-fluoro-5-(7-fluoro-1-oxo-1,2,3,4-tetrahydroisoquinolin-6-yl)pyridin-3-yl)-2-(trifluoromethyl)phenyl)piperazine-1-carboxylate (114.4 mg, 0.190 mmol), TFA (2 mL, 26.1 mmol) and CH₂Cl₂ (4 mL) afforded the title compound (71.7 mg, 71%) as a beige powder. ¹H NMR (500 MHz, DMSO-d₆) δ 8.11 (br s, 1H), 7.80 (br d, J=8.6 Hz, 1H), 7.76 (s, 1H), 7.73 (d, J=10.3 Hz, 1H), 7.61 (d, J=10.0 Hz, 1H), 7.53 (d, J=8.4 Hz, 1H), 7.43 (d, J=7.1 Hz, 1H), 6.41 (s, 2H), 3.40 (dt, J=6.5, 2.7 Hz, 2H), 2.92 (br t, J=6.4 Hz, 2H), 2.80 (br d, J=4.6 Hz, 8H), NH not shown; LCMS: [M+H]⁺=504.45.

### Example 220: 6-(2-amino-6-fluoro-5-(4-((1R,5S)-3-(2-methoxyacetyl)-3-azabicyclo[3.1.0]hexan-1-yl)phenyl)pyridin-3-yl)-3,4-dihydroisoquinolin-1(2H)-one (I-220)

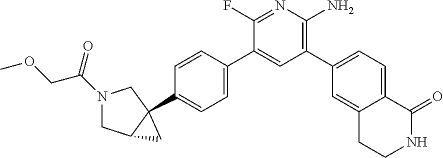 [SMILES: COCC(=O)N1C[C@H]2C[C@@]2(c2ccc(-c3cc(-c4ccc5c(c4)CCNC5=O)c(N)nc3F)cc2)C1]

Step 1: 6-(5-(4-((1R,5S)-3-azabicyclo[3.1.0]hexan-1-yl)phenyl)-2-amino-6-fluoropyridin-3-yl)-3,4-dihydroisoquinolin-1(2H)-one

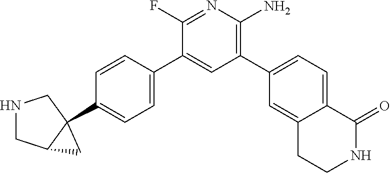 [SMILES: Nc1nc(F)c(-c2ccc([C@]34CNC[C@H]3C4)cc2)cc1-c1ccc2c(c1)CCNC2=O]

A procedure similar to Example 1, General Method A by heating a sealed, degassed mixture of (1R,5S)-1-(4-bromophenyl)-3-azabicyclo[3.1.0]hexane (300 mg, 1.26 mmol), 6-(2-amino-6-fluoro-5-(4,4,5,5-tetramethyl-1,3,2-dioxaborolan-2-yl)pyridin-3-yl)-3,4-dihydroisoquinolin-1(2H)-one (579 mg, 1.51 mmol), PdCl₂dppf (92 mg, 0.13 mmol), Cs₂CO₃ (1231 mg, 3.78 mmol), DME (10 mL) and H₂O (5 mL) in a microwave reactor at 90° C. for 2 h afforded the title compound as a tan solid (298 mg, 57%). LCMS: [M+H]⁺=415.44.

Step 2: 6-(2-amino-6-fluoro-5-(4-((1R,5S)-3-(2-methoxyacetyl)-3-azabicyclo[3.1.0]hexan-1-yl)phenyl)pyridin-3-yl)-3,4-dihydroisoquinolin-1(2H)-one

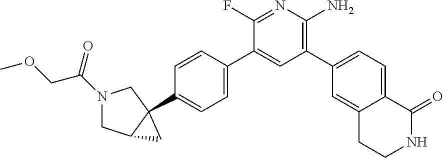 [SMILES: COCC(=O)N1C[C@H]2C[C@@]2(c2ccc(-c3cc(-c4ccc5c(c4)CCNC5=O)c(N)nc3F)cc2)C1]

To a CH₂Cl₂ (15 mL) solution of 6-(5-(4-((1R,5S)-3-azabicyclo[3.1.0]hexan-1-yl)phenyl)-2-amino-6-fluoropyridin-3-yl)-3,4-dihydroisoquinolin-1(2H)-one (60 mg, 0.14 mmol) and i-Pr₂NEt (0.063 mL, 0.36 mmol) was added a CH₂Cl₂ solution of methoxyacetyl chloride (22.9 mg/mL in CH₂Cl₂, 0.68 mL, 0.14 mmol) at 0° C. and the reaction was allowed to slowly warm to RT and then kept stirred overnight. An excess of 2 M NH3/MeOH was added and after briefly stirring at RT, the reaction was concentrated under reduced pressure and purified by flash chromatography (25 g SiO₂, using MeOH in CH₂Cl₂) to afford the title compound (63 mg, 89%) as a light beige solid. ¹H NMR (500 MHz, DMSO-d₆) δ 7.95 (br s, 1H), 7.91 (d, J=7.8 Hz, 1H), 7.64 (d, J=10.0 Hz, 1H), 7.41-7.56 (m, 4H), 7.28 (d, J=7.2 Hz, 2H), 6.32 (s, 2H), 4.00-4.13 (m, 2H), 3.94-4.00 (m, 1H), 3.66-3.82 (m, 1H), 3.64-3.66 (m, 1H), 3.38-3.57 (m, 3H), 3.29-3.32 (m, 3H), 2.96 (br t, J=6.5 Hz, 2H), 1.94-2.13 (m, 1H), 1.07-1.24 (m, 1H), 0.78 (t, J=4.6 Hz, 1H). LCMS: [M+H]⁺=487.52.

### Example 221: 6-(6′-(4-acetylpiperazin-1-yl)-6-amino-2-fluoro-[3,3′-bipyridin]-5-yl)-7-fluoro-3,4-dihydroisoquinolin-1(2H)-one 2,2,2-trifluoroacetate (1-221)

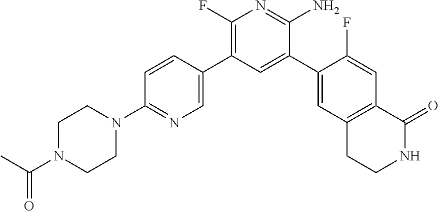 [SMILES: CC(=O)N1CCN(c2ccc(-c3cc(-c4cc5c(cc4F)C(=O)NCC5)c(N)nc3F)cn2)CC1]

A method similar to Example 1, General Method A heating a sealed, degassed mixture of 2-(4-acetylpiperazin-1-yl)pyridine-5-boronic acid pinacol ester (66 mg, 0.19 mmol), PdCl₂dppf (12 mg, 0.016 mmol), 6-(2-amino-5-bromo-6-fluoropyridin-3-yl)-7-fluoro-3,4-dihydroisoquinolin-1(2H)-one (67 mg, 0.13 mmol, 71% purity), Cs₂CO₃ (153 mg, 0.470 mmol), H₂O (2.0 mL) and DME (4 mL) afforded the title compound as a clear film (48.8 mg 61%). ¹H NMR (500 MHz, CD₃OD) δ 8.25 (br d, J=9.3 Hz, 1H), 8.16 (s, 1H), 7.74 (br d, J=8.3 Hz, 2H), 7.43 (br d, J=9.4 Hz, 2H), 3.85 (br s, 6H), 3.76-3.82 (m, 2H), 3.55 (br t, J =5.9 Hz, 2H), 3.03 (br s, 2H), 2.18 (s, 3H). LCMS: [M+H]⁺=479.47.

### Example 222: 6-(6-amino-2-fluoro-5-(1-oxo-1,2,3,4-tetrahydroisoquinolin-6-yl)pyridin-3-yl)-1′-isopropylspiro[chromane-2,4′-piperidin]-4-one (I-222)

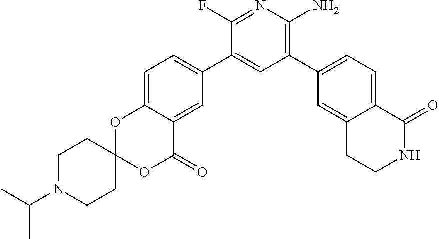 [SMILES: CC(C)N1CCC2(CC1)OC(=O)c1cc(-c3cc(-c4ccc5c(c4)CCNC5=O)c(N)nc3F)ccc1O2]

A procedure similar to Example 218 using 6-(6-amino-2-fluoro-5-(1-oxo-1,2,3,4-tetrahydroisoquinolin-6-yl)pyridin-3-yl)spiro[chromane-2,4′-piperidin]-4-one (29.4 mg, 0.062 mmol) in CH₂Cl₂ (1.5 mL), acetone (1.5 mL) was added 2 drops of glacial AcOH (24 mg, 0.400 mmol) and NaH(OAc)₃ (50 mg, 0.236 mmol) afforded the title compound (27.4 mg, 81%) as a beige powder. ¹H NMR (500 MHz, DMSO-d₆) δ 7.95 (br s, 1H), 7.90 (d, J=7.9 Hz, 1H), 7.87 (s, 1H), 7.80 (br d, J=8.7 Hz, 1H), 7.64 (d, J=10.1 Hz, 1H), 7.47 (d, J=7.9 Hz, 1H), 7.45 (s, 1H), 7.15 (br d, J=7.8 Hz, 1H), 6.33 (s, 2H), 3.44-3.39 (m, 6H), 2.95 (br t, J=6.5 Hz, 2H), 2.87 (br s, 3H), 2.12-1.97 (m, 2H), 1.91 (s, 3H), 1.89-1.70 (m, 2H), 1.11 (br s, 6H); LCMS: [M+H]⁺=515.58.

### Example 223: 6-(2-amino-6-fluoro-5-(1′-isopropylspiro[chromane-2,4′-piperidin]-6-yl)pyridin-3-yl)-3,4-dihydroisoquinolin-1(2H)-one (I-223)

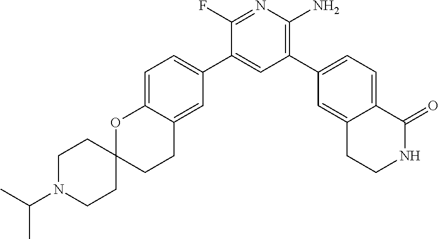 [SMILES: CC(C)N1CCC2(CCc3cc(-c4cc(-c5ccc6c(c5)CCNC6=O)c(N)nc4F)ccc3O2)CC1]

A procedure similar to Example 218 using 6-(2-amino-6-fluoro-5-(spiro[chromane-2,4′-piperidin]-6-yl)pyridin-3-yl)-3,4-dihydroisoquinolin-1(2H)-one (29.4 mg, 0.064 mmol) in CH₂Cl₂ (1.5 mL), acetone (1.5 mL), glacial AcOH (24 mg, 0.400 mmol) and NaBH(OAc)₃ (50 mg, 0.236 mmol) afforded the title compound (15.4 mg, 46%) as a white powder. ¹H NMR (500 MHz, DMSO-d₆) δ 7.95 (br s, 1H), 7.90 (d, J=7.9 Hz, 1H), 7.58 (d, J=10.1 Hz, 1H), 7.48-7.42 (m, 2H), 7.32-7.25 (m, 2H), 6.87 (br s, 1H), 6.21 (s, 2H), 3.44-3.38 (m, 5H), 2.95 (br t, J=6.5 Hz, 2H), 2.79 (br s, 2H), 2.06-1.93 (m, 2H), 1.91 (s, 3H), 1.82 (br s, 2H), 1.24 (br d, J=11.2 Hz, 6H); LCMS: [M+H]⁺=501.59.

### Example 224: 5-(6-amino-2-fluoro-5-(1-oxo-1,2,3,4-tetrahydroisoquinolin-6-yl)pyridin-3-yl)-2-((1-isopropylpiperidin-4-yl)oxy)benzonitrile (I-224)

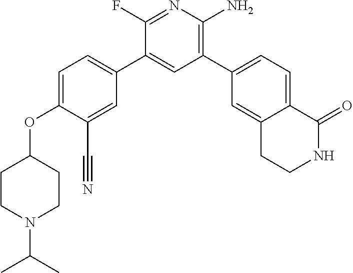 [SMILES: CC(C)N1CCC(Oc2ccc(-c3cc(-c4ccc5c(c4)CCNC5=O)c(N)nc3F)cc2C#N)CC1]

A procedure similar to Example 218 using 5-(6-amino-2-fluoro-5-(1-oxo-1,2,3,4-tetrahydroisoquinolin-6-yl)pyridin-3-yl)-2-(piperidin-4-yloxy)benzonitrile (26.1 mg, 0.057 mmol) in CH₂Cl₂ (1.5 mL), acetone (1.5 mL), 2 drops of glacial AcOH (24 mg, 0.400 mmol) and NaBH(OAc)₃ (50 mg, 0.236 mmol) afforded the title compound (14.2 mg, 47%) as a beige powder. ¹H NMR (500 MHz, DMSO-d₆) δ 7.95 (br s, 2H), 7.91 (d, J=7.9 Hz, 1H), 7.84 (br d, J=8.1 Hz, 1H), 7.71 (d, J=10.1 Hz, 1H), 7.48 (br d, J=8.2 Hz, 1H), 7.45 (s, 1H), 7.39 (br d, J=9.0 Hz, 1H), 6.39 (br s, 2H), 5.06-4.60 (m, 1H), 3.46-3.38 (m, 6H), 2.95 (br t, J=6.4 Hz, 3H), 2.32-2.06 (m, 4H), 1.91 (s, 3H), 1.30-1.07 (m, 6H); LCMS: [M+H]⁺=500.54.

### Example 225: 6-(2-amino-5-(4-((1R,5S)-3-(dimethylglycyl)-3-azabicyclo[3.1.0]hexan-1-yl)phenyl)-6-fluoropyridin-3-yl)-3,4-dihydroisoquinolin-1(2H)-one (I-225)

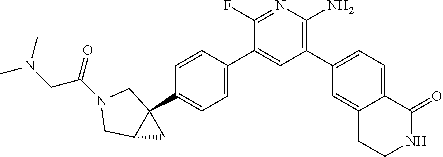 [SMILES: CN(C)CC(=O)N1C[C@H]2C[C@@]2(c2ccc(-c3cc(-c4ccc5c(c4)CCNC5=O)c(N)nc3F)cc2)C1]

To a CH₂Cl₂ (15 mL) solution of 6-(5-(4-((1R,5S)-3-azabicyclo[3.1.0]hexan-1-yl)phenyl)-2-amino-6-fluoropyridin-3-yl)-3,4-dihydroisoquinolin-1(2H)-one (60 mg, 0.14 mmol) and i-Pr₂NEt (0.063 mL, 0.36 mmol) was added 2-(dimethylamino)acetyl chloride·HCl (23 mg, 0.14 mmol) as solid in one portion at 0° C. The reaction was allowed to warm to rt and stirring was continued overnight. Later, i-Pr₂NEt (0.15 mL, 0.87 mmol) was added followed by 2-(dimethylamino)acetylchloride·HCl (75 mg, 0.48 mmol) at 0° C. After stirring at 0° C. for 2 h excess 2 M NH3/MeOH was added. The reaction mixture was concentrated and purified by flash chromatography (25 g SiO₂, using CH₂Cl₂/MeOH/aq NH₄OH 89/10/1 in CH₂Cl₂) followed by a filtration through a Waters PoraPak CX column to afford the title compound (27.2 mg, 38%) as a cream white solid. ¹H NMR (500 MHz, DMSO-d₆) δ 7.95 (br s, 1H), 7.91 (d, J=7.8 Hz, 1H), 7.60-7.67 (m, 1H), 7.37-7.56 (m, 4H), 7.17-7.33 (m, 2H), 6.31 (br s, 2H), 4.02-4.21 (m, 1H), 3.88 (d, J=10.4 Hz, 0.5 H), 3.77 (d, J=11.5 Hz, 1H), 3.69 (dd, J=10.4, 3.9 Hz, 0.5H), 3.39-3.53 (m, 3H), 3.06-3.26 (m, 1H), 2.80-3.05 (m, 3H), 2.22 (d, J=2.1 Hz, 6H), 1.91-2.09 (m, 1H), 1.07-1.14 (m, 1H), 0.74 (t, J=4.4 Hz, 1H). LCMS: [M+H]⁺=500.54.

### Example 226: 6-(6-amino-2-fluoro-6′-(4-isopropylpiperazin-1-yl)-[3,3′-bipyridin]-5-yl)-7-fluoro-3,4-dihydroisoquinolin-1(2H)-one (I-226)

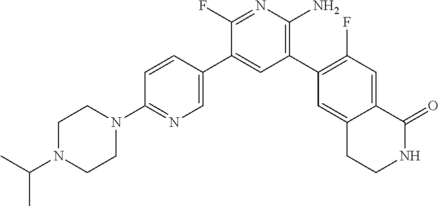 [SMILES: CC(C)N1CCN(c2ccc(-c3cc(-c4cc5c(cc4F)C(=O)NCC5)c(N)nc3F)cn2)CC1]

Step 1: 6-(2-amino-5-bromo-6-fluoropyridin-3-yl)-7-fluoro-3,4-dihydroisoquinolin-1(2H)-one

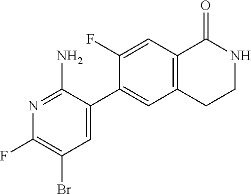 [SMILES: Nc1nc(F)c(Br)cc1-c1cc2c(cc1F)C(=O)NCC2]

A procedure similar to Example 1, General Method A, by heating a degassed, sealed mixture of H₂O (6 mL), 5-bromo-6-fluoro-3-iodopyridin-2-amine (500 mg, 1.58 mmol), 7-fluoro-6-(4,4,5,5-tetramethyl-1,3,2-dioxaborolan-2-yl)-3,4-dihydroisoquinolin-1(2H)-one (505 mg, 1.74 mmol), Na₂CO₃ (334 mg, 3.16 mmol), Pd (PPh₃)₂Cl₂ (111 mg, 0.158 mmol) and MeCN (12 mL) in a microwave reactor at 80° C. for 45 min and then again for 1 h, afforded, after workup and purification, the product as a brown solid (290 mg, 37% yield based on purity of 71%). LCMS: [M+H]⁺=354.34/356.30.

Step 2: 6-(6-amino-2-fluoro-6′-(piperazin-1-yl)-[3,3′-bipyridin]-5-yl)-7-fluoro-3,4-dihydroisoquinolin-1(2H)-one

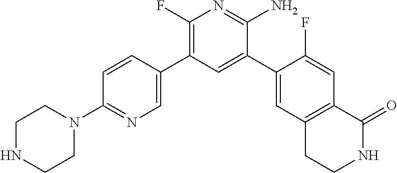 [SMILES: Nc1nc(F)c(-c2ccc(N3CCNCC3)nc2)cc1-c1cc2c(cc1F)C(=O)NCC2]

A procedure similar to Example 1, General Method A using 6-(2-amino-5-bromo-6-fluoropyridin-3-yl)-7-fluoro-3,4-dihydroisoquinolin-1(2H)-one (80 mg, 0.16 mmol), tert-butyl 4-(5-(4,4,5,5-tetramethyl-1,3,2-dioxaborolan-2-yl)pyridin-2-yl)piperazine-1-carboxylate (75 mg, 0.19 mmol, 71% purity), PdCl₂dppf (12 mg, 0.016 mmol), Cs₂CO₃ (157 mg, 0.481 mmol), H₂O (2.0 mL) and DME (4 mL) afforded the product as a light tan solid (64 mg, 65% yield based on purity of 71%). LCMS [M+H]⁺=437.49.

Step 3: 6-(6-amino-2-fluoro-6′-(4-isopropylpiperazin-1-yl)-[3,3′-bipyridin]-5-yl)-7-fluoro-3,4-dihydroisoquinolin-1(2H)-one

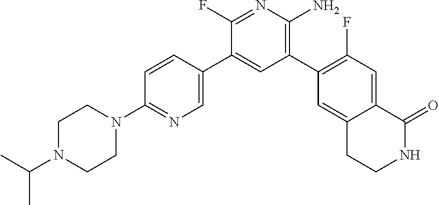 [SMILES: CC(C)N1CCN(c2ccc(-c3cc(-c4cc5c(cc4F)C(=O)NCC5)c(N)nc3F)cn2)CC1]

A solution of 6-(6-amino-2-fluoro-6′-(piperazin-1-yl)-[3,3′-bipyridin]-5-yl)-7-fluoro-3,4-dihydroisoquinolin-1(2H)-one (65 mg, 0.106 mmol, 79% purity) in anh. CH₂Cl₂ (15 mL) was treated with acetone (0.039 mL, 0.53 mmol) at RT and glacial AcOH (0.024 mL, 0.42 mmol). The mixture was stirred at RT for 15 min before NaBH(OAc)₃ (90 mg, 0.42 mmol) was added in one portion at RT. Stirring was continued for 2 d. Later, acetone (0.039 mL, 0.53 mmol) and after 3 h, NaBH(OAc)₃ (90 mg, 0.42 mmol) were added in sequence. After 1 d at of stirring at RT, MeOH was added and the mixture was concentrated and purified by preparative HPLC (30 g Biotage® SNAP KP-C₁₈-HS, MeOH in (H₂O+0.05% TFA)) followed by a filtration through a Waters PoraPak CX column to afford the title compound as a very pale yellow solid (23.0 mg, 51% yield based on purity of 98%). ¹H NMR (500 MHz, DMSO-d₆) δ 8.28 (s, 1H), 8.11 (br s, 1H), 7.71 (br d, J=8.7 Hz, 1H), 7.62 (t, J=10.0 Hz, 2H), 7.43 (d, J=7.0 Hz, 1H), 6.88 (br d, J=8.8 Hz, 1H), 6.25 (s, 2H), 3.49 (br d, J=10.9 Hz, 4H), 3.39-3.45 (m, 3H), 2.93 (br t, J=6.4 Hz, 2H), 2.74 (br s, 1H), 2.54-2.66 (m, 3H), 0.96-1.12 (m, 6H). LCMS: [M+H]⁺=479.54.

### Example 227: 6-(2-amino-5-(3-amino-8-chloro-7-fluoroisoquinolin-6-yl)-6-fluoropyridin-3-yl)-3,4-dihydroisoquinolin-1(2H)-one (I-227)

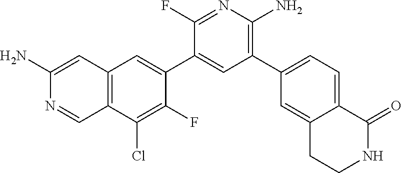 [SMILES: Nc1cc2cc(-c3cc(-c4ccc5c(c4)CCNC5=O)c(N)nc3F)c(F)c(Cl)c2cn1]

A degassed solution mixture of 8-chloro-7-fluoro-6-iodoisoquinolin-3-amine (40 mg, 0.124 mmol), 6-(2-amino-6-fluoro-5-(4,4,5,5-tetramethyl-1,3,2-dioxaborolan-2-yl)pyridin-3-yl)-3,4-dihydroisoquinolin-1(2H)-one (71.3 mg, 0.186 mmol), and bis(4-(di-tert-butylphosphanyl)-N,N′dimethylaniline)-palladium chloride (17.56 mg, 0.025 mmol) in 1,4-dioxane (10 mL) and 1 M aqueous K₃PO₄ solution (4.00 mL) was agitated at 90° C. over 60 min. The mixture was concentrated and extracted with EtOAc/water, the organic layer was concentrated and purified by silica gel chromatography (13 g column, eluting with 0-5-100% MeOH/water) to afford the title compound (11 mg, 19%) as a yellow powder. ¹H NMR (500 MHz, DMSO-d₆) δ 8.95-9.03 (m, 1H), 7.93-7.97 (m, 1H), 7.89-7.93 (m, 1H), 7.70-7.74 (m, 1H), 7.65-7.70 (m, 1H), 7.43-7.50 (m, 2H), 6.67-6.72 (m, 1H), 6.51-6.62 (m, 2H), 6.18-6.27 (m, 2H), 3.36-3.45 (m, 2H), 2.91-3.00 (m, 2H); LCMS: [M+H]⁺=452.46.

### Example 228: 2-(6-amino-2-fluoro-5-(1-oxo-1,2,3,4-tetrahydroisoquinolin-6-yl)pyridin-3-yl)-6-isopropyl-5,6-dihydro-4H-pyrazolo[1,5-d][1,4]diazepin-7(8H)-one (I-228)

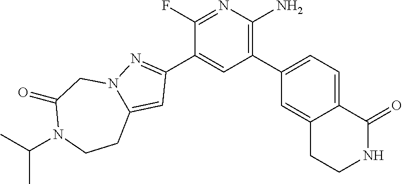 [SMILES: CC(C)N1CCc2cc(-c3cc(-c4ccc5c(c4)CCNC5=O)c(N)nc3F)nn2CC1=O]

A procedure analogous to Example 43, Step 5 using 2-bromo-6-isopropyl-5,6-dihydro-4H-pyrazolo[1,5-d][1,4]diazepin-7(8H)-one (35 mg, 0.129 mmol), 6-(2-amino-6-fluoro-5-(4,4,5,5-tetramethyl-1,3,2-dioxaborolan-2-yl)pyridin-3-yl)-3,4-dihydroisoquinolin-1(2H)-one (73.9 mg, 0.193 mmol) and bis(di-tert-butyl(4-dimethylaminophenyl)phosphine)dichloropalladium(II) (91 mg, 0.129 mmol) in 1,4-dioxane (10 ml) and K₃PO₄ (2.00 mL of a 1.0 M aqueous solution) afforded the title compound (14 mg, 23%), as a white powder. ¹H NMR (500 MHz, DMSO-d₆) δ 7.81-8.02 (m, 2H), 7.36-7.46 (m, 2H), 6.21-6.45 (m, 2H), 4.98-5.09 (m, 2H), 4.50-4.64 (m, 1H), 3.73-3.87 (m, 2H), 3.38-3.47 (m, 2H), 3.01-3.08 (m, 1H), 2.90-3.01 (m, 2H), 1.12 (s, 6H); LCMS: [M+H]⁺=449.45.

### Example 229: 2-(6-amino-2-fluoro-5-(1-oxo-1,2,3,4-tetrahydroisoquinolin-6-yl)pyridin-3-yl)-5,6-dihydro-4H-pyrazolo[1,5-d][1,4]diazepin-7(8H)-one (I-229)

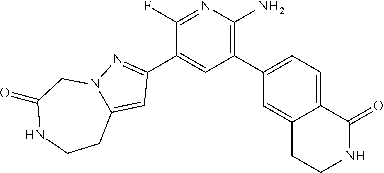 [SMILES: Nc1nc(F)c(-c2cc3n(n2)CC(=O)NCC3)cc1-c1ccc2c(c1)CCNC2=O]

A procedure similar to Example 43, Step 5 using 2-bromo-5,6-dihydro-4H-pyrazolo[1,5-d][1,4]diazepin-7(8H)-one (35 mg, 0.152 mmol)), 6-(2-amino-6-fluoro-5-(4,4,5,5-tetramethyl-1,3,2-dioxaborolan-2-yl)pyridin-3-yl)-3,4-dihydroisoquinolin-1(2H)-one (87 mg, 0.228 mmol), and bis(di-tert-butyl(4-dimethylaminophenyl)phosphine)dichloropalladium(II) (21.54 mg, 0.030 mmol) in 1,4-dioxane (10 mL) and K₃PO₄ (2.00 mL of a 1.0 M aqueous solution) afforded the title compound (4.4 mg, 7%) as a white powder. ¹H NMR (500 MHz, DMSO-d₆) δ 8.09-8.25 (m, 1H), 7.82-8.01 (m, 3H), 7.37-7.49 (m, 2H), 6.25-6.44 (m, 3H), 5.72 (s, 2H), 4.92 (s, 2H), 3.94-4.26 (m, 4H), 2.99-3.06 (m, 2H), 2.93-3.00 (m, 2H); LCMS: [M+H]⁺=407.62

### Example 230: 6-(2-amino-6-chloro-5-(4-(1-methyl-1,2,3,6-tetrahydropyridin-4-yl)phenyl)pyridin-3-yl)-3,4-dihydroisoquinolin-1(2H)-one (I-230)

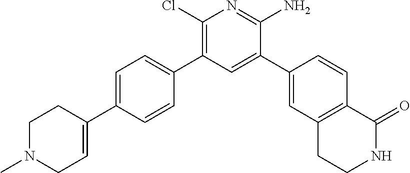 [SMILES: CN1CC=C(c2ccc(-c3cc(-c4ccc5c(c4)CCNC5=O)c(N)nc3Cl)cc2)CC1]

A procedure analogous to that of Example 43, step 5, by coupling 6-(2-amino-5-bromo-6-chloropyridin-3-yl)-3,4-dihydroisoquinolin-1(2H)-one (40 mg, 0.113 mmol) and 4-(1-methyl-1,2,3,6-tetrahydropyridin-4-yl)phenylboronic acid pinacol ester (44.1 mg, 0.147 mmol), [1,12-bis(diphenylphosphino)ferrocene]dichloropalladium(II) (12.45 mg, 0.017 mmol) and Cs₂CO₃ (111 mg, 0.340 mmol) in DME (3.5 mL) afforded the title compound as a beige solid (13 mg, 24%). ¹H NMR (500 MHz, DMSO-d₆) δ 7.9-8.0 (m, 1H), 7.9-7.9 (m, 1H), 7.4-7.5 (m, 6H), 7.4-7.4 (m, 1H), 6.3-6.4 (m, 2H), 6.1-6.3 (m, 1H), 3.4-3.4 (m, 2H), 3.1-3.1 (m, 2H), 2.9-3.0 (m, 2H), 2.6-2.7 (m, 2H), 2.5-2.6 (m, 2H), 2.3-2.4 (m, 3H); LCMS: [M+H]⁺=445.54.

### Example 231: 6-(2-amino-6-chloro-5-(4-(1-methylpiperidin-4-yl)phenyl)pyridin-3-yl)-3,4-dihydroisoquinolin-1(2H)-one (I-231)

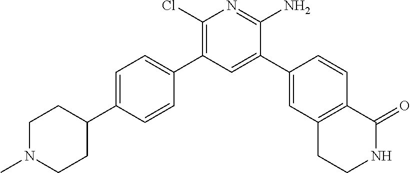 [SMILES: CN1CCC(c2ccc(-c3cc(-c4ccc5c(c4)CCNC5=O)c(N)nc3Cl)cc2)CC1]

A procedure analogous to that of Example 43, step 5 using 6-(2-amino-5-bromo-6-chloropyridin-3-yl)-3,4-dihydroisoquinolin-1(2H)-one (40 mg, 0.113 mmol) and 1-methyl-4-(4-(4,4,5,5-tetramethyl-1,3,2-dioxaborolan-2-yl)phenyl)piperidine (44.4 mg, 0.147 mmol), [1,12-bis(diphenylphosphino)ferrocene]dichloropalladium(II) (12.45 mg, 0.017 mmol) and Cs₂CO₃ (111 mg, 0.340 mmol) in DME (3.5 mL) afforded the title compound (26 mg, 49%) as a beige solid. ¹H NMR (500 MHz, DMSO-d₆) δ 7.9-8.0 (m, 1H), 7.9-7.9 (m, 1H), 7.4-7.5 (m, 2H), 7.4-7.4 (m, 3H), 7.3-7.3 (m, 2H), 6.3-6.4 (m, 2H), 3.4-3.4 (m, 2H), 2.9-3.0 (m, 2H), 2.8-2.9 (m, 2H), 2.4-2.5 (m, 1H), 2.2-2.2 (m, 3H), 1.9-2.0 (m, 2H), 1.7-1.8 (m, 4H); LCMS: [M+H]⁺=447.67.

### Example 232: 6-(2-amino-6-chloro-5-(4-(1-methyl-1,2,3,6-tetrahydropyridin-4-yl)phenyl)pyridin-3-yl)-7-fluoro-3,4-dihydroisoquinolin-1(2H)-one (I-232)

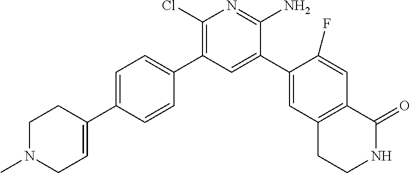 [SMILES: CN1CC=C(c2ccc(-c3cc(-c4cc5c(cc4F)C(=O)NCC5)c(N)nc3Cl)cc2)CC1]

A procedure analogous to that of Example 43, Step 5, 6-(2-amino-5-bromo-6-chloropyridin-3-yl)-7-fluoro-3,4-dihydroisoquinolin-1(2H)-one (40 mg, 0.108 mmol), 4-(1-methyl-1,2,3,6-tetrahydropyridin-4-yl)phenylboronic acid pinacol ester (42.0 mg, 0.140 mmol), [1,12-bis(diphenylphosphino)ferrocene]dichloropalladium(II) (11.85 mg, 0.016 mmol) and Cs₂CO₃ (105 mg, 0.324 mmol) in DME (3.5 mL) afforded the title compound as a yellow powder (17 mg, 31%). ¹H NMR (500 MHz, DMSO-d₆) δ 8.1-8.2 (m, 1H), 7.6-7.6 (m, 1H), 7.4-7.5 (m, 2H), 7.4-7.4 (m, 4H), 6.35 (s, 2H), 6.2-6.3 (m, 1H), 3.4-3.5 (m, 4H), 3.0-3.1 (m, 2H), 2.9-2.9 (m, 2H), 2.6-2.6 (m, 2H), 2.3-2.3 (m, 3H); LCMS: [M+H]⁺=463.53.

### Example 233: 6-(2-amino-6-chloro-5-(4-(1-methylpiperidin-4-yl)phenyl)pyridin-3-yl)-7-fluoro-3,4-dihydroisoquinolin-1(2H)-one (I-233)

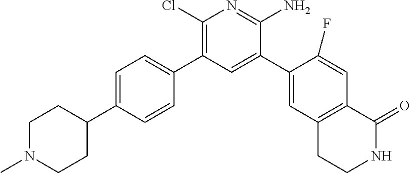 [SMILES: CN1CCC(c2ccc(-c3cc(-c4cc5c(cc4F)C(=O)NCC5)c(N)nc3Cl)cc2)CC1]

A procedure analogous to that of Example 43, step 5 using 6-(2-amino-5-bromo-6-chloropyridin-3-yl)-7-fluoro-3,4-dihydroisoquinolin-1(2H)-one (40 mg, 0.108 mmol), 1-methyl-4-(4-(4,4,5,5-tetramethyl-1,3,2-dioxaborolan-2-yl)phenyl)piperidine (42.3 mg, 0.140 mmol), [1,12-bis(diphenylphosphino)ferrocene]dichloropalladium(II) (11.85 mg, 0.016 mmol) and Cs₂CO₃ (105 mg, 0.324 mmol) in DME (3.5 mL) and H₂O (1 mL) afforded the title compound (17 mg, 31%) as a yellow powder. ¹H NMR (500 MHz, DMSO-d6) δ 8.1-8.2 (m, 1H), 7.6-7.6 (m, 1H), 7.4-7.5 (m, 1H), 7.3-7.4 (m, 3H), 7.2-7.3 (m, 2H), 6.2-6.4 (m, 2H), 3.4-3.4 (m, 2H), 2.8-2.9 (m, 4H), 2.4-2.5 (m, 1H), 2.2-2.2 (m, 3H), 1.9-2.1 (m, 2H), 1.7-1.8 (m, 2H), 1.6-1.7 (m, 2H); LCMS: [M+H]⁺=465.60.

### Example 234: 6-(5,6-diamino-2-fluoro-4′-methyl-[3,3′-bipyridin]-5-yl)-3,4-dihydroisoquinolin-1(2H)-one 2,2,2-trifluoroacetate (1-234)

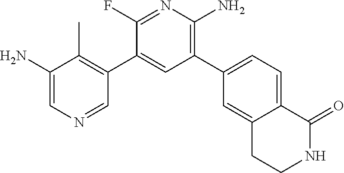 [SMILES: Cc1c(N)cncc1-c1cc(-c2ccc3c(c2)CCNC3=O)c(N)nc1F]

Step 1: bis-tert-butyl (6′-amino-2′-fluoro-4-methyl-5′-(1-oxo-1,2,3,4-tetrahydroisoquinolin-6-yl)-[3,3′-bipyridin]-5-yl)carbamate

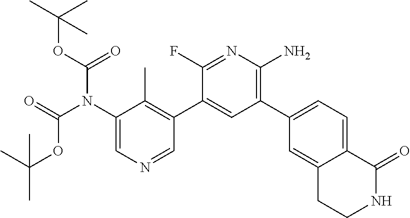 [SMILES: Cc1c(-c2cc(-c3ccc4c(c3)CCNC4=O)c(N)nc2F)cncc1N(C(=O)OC(C)(C)C)C(=O)OC(C)(C)C]

A degassed solution mixture of bis-tert-butyl (5-bromo-4-methylpyridin-3-yl)carbamate (40 mg, 0.103 mmol), 6-(2-amino-6-fluoro-5-(4,4,5,5-tetramethyl-1,3,2-dioxaborolan-2-yl)pyridin-3-yl)-3,4-dihydroisoquinolin-1(2H)-one (59.4 mg, 0.155 mmol) and bis(di-tert-butyl(4-dimethylaminophenyl)phosphine)dichloropalladium(II) (14.63 mg, 0.021 mmol) in 1,4-Dioxane (10 mL) and aqueous K₃PO₄ (2.0 mL of a 1.0 M solution) was agitated at 90° C. for 60 min. The mixture was concentrated and partitioned between EtOAc and water. The separated organic layer was concentrated and purified by column chromatography (13 g reverse phase column, 0-5-100% ACN/water) to afford the product (26 mg, 45% yield), as a white powder. LCMS: [M+H]⁺=564.57.

Step 2: 6-(5′,6-diamino-2-fluoro-4′-methyl-[3,3′-bipyridin]-5-yl)-3,4-dihydroisoquinolin-1(2H)-one, trifluoroacetic acid salt

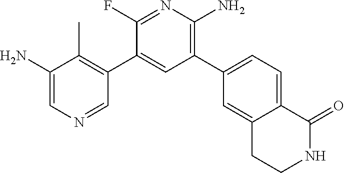 [SMILES: Cc1c(N)cncc1-c1cc(-c2ccc3c(c2)CCNC3=O)c(N)nc1F]

A mixture of bis-tert-butyl (6′-amino-2′-fluoro-4-methyl-5′-(1-oxo-1,2,3,4-tetrahydroisoquinolin-6-yl)-[3,3′-bipyridin]-5-yl)carbamate (26 mg, 0.046 mmol) in 1:1 TFA/DCM (2 mL) was stirred at 25° C. for 2 h and then concentrated and purified by silica gel chromatography (eluting with DCM/MeOH 0-5-30%) to afford, after lyophilization, the TFA salt of the title compound (12 mg, 23%) as a white powder. ¹H NMR (500 MHz, CD₃OD) δ 7.90-7.97 (m, 1H), 7.83-7.90 (m, 1H), 7.71-7.81 (m, 1H), 7.38-7.43 (m, 2H), 7.34-7.37 (m, 1H), 3.40-3.53 (m, 2H), 2.87-3.03 (m, 2H), 2.04-2.15 (m, 3H); LCMS: [M+H]⁺=364.55.

### Example 235: 6-(2-amino-5-(4-(1-ethylpyrrolidin-3-yl)phenyl)-6-fluoropyridin-3-yl)-3,4-dihydroisoquinolin-1(2H)-one (I-235)

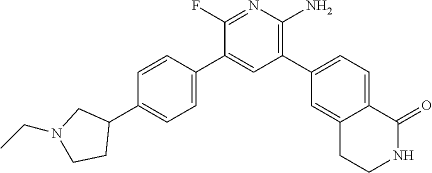 [SMILES: CCN1CCC(c2ccc(-c3cc(-c4ccc5c(c4)CCNC5=O)c(N)nc3F)cc2)C1]

A degassed mixture of 6-(2-amino-5-(4-(2,5-dihydro-1 H-pyrrol-3-yl)phenyl)-6-fluoropyridin-3-yl)-3,4-dihydroisoquinolin-1(2H)-one (60 mg, 0.15 mmol) and Pd/C (10 wt %, 16 mg, 0.015 mmol) in EtOH (10 mL) was stirred under H₂ (1 atm) at RT for 3 h. The mixture was filtered through Celite® using MeOH—CH₂Cl₂. Concentration of the filtrate afforded 6-(2-amino-6-fluoro-5-(4-(pyrrolidin-3-yl)phenyl)pyridin-3-yl)-3,4-dihydroisoquinolin-1(2H)-one. This material was taken up in anh. DMF (2 mL) and treated with Et₃N (0.21 mL, 1.5 mmol) and ethyl bromide (0.022 mL, 0.30 mmol) at RT. The mixture was stirred at RT for 18 h, concentrated and purified by flash chromatography (25 g SiO₂, using MeOH in CH₂Cl₂ 0 to 14% then 2 M NH₃/MeOH in CH₂Cl₂) followed by a filtration through a Waters PoraPak CX column to afford the title compound as a white solid (19.0 mg, 30% yield for 2 steps). ¹H NMR (500 MHz, DMSO-d₆) δ 7.95 (br s, 1H), 7.91 (d, J=8.0 Hz, 1H), 7.63 (d, J=10.2 Hz, 1H), 7.44-7.50 (m, 4H), 7.33 (d, J=8.2 Hz, 2H), 6.29 (s, 2H), 3.39-3.50 (m, 4H), 2.91-3.01 (m, 3H), 2.60-2.77 (m, 2H), 2.43-2.49 (m, 2H), 2.21-2.30 (m, 1H), 1.73-1.82 (m, 1H), 1.07 (t, J=7.2 Hz, 3H); LCMS: [M+H]⁺=431.54.

### Example 236: 6-(2-amino-5-(4-(4-(2,2-difluoroethyl)piperazin-1-yl)phenyl)-6-fluoropyridin-3-yl)-7-fluoro-3,4-dihydroisoquinolin-1(2H)-one (I-236)

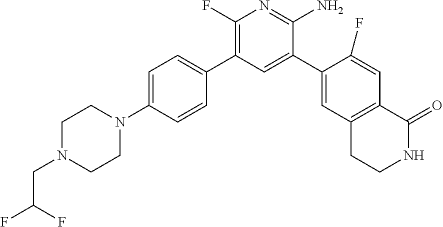 [SMILES: Nc1nc(F)c(-c2ccc(N3CCN(CC(F)F)CC3)cc2)cc1-c1cc2c(cc1F)C(=O)NCC2]

To a solution of 6-(2-amino-6-fluoro-5-(4-(piperazin-1-yl)phenyl)pyridin-3-yl)-7-fluoro-3,4-dihydroisoquinolin-1(2H)-one, trifluoroacetate (25 mg, 0.045 mmol) and N,N-diisopropylethylamine (0.079 ml, 0.455 mmol) in DMF (1.0 mL) was added 2,2-difluoroethyl trifluoromethanesulfonate (14.61 mg, 0.068 mmol). The reaction was stirred at RT for 1 h, concentrated in vacuo and purified by Waters prep HPLC (XBridge Prep C18 5 μm, 10×100 mm Column, 80-70% 0.1% formic acid in water/0.1% formic acid in acetonitrile) to afford the title compound (13.6 mg, 60% yield) as an off-white solid. ¹H NMR (500 MHz, DMSO-d₆) δ 8.11 (br s, 1H), 7.60 (dd, J=12.5, 10.2 Hz, 2H), 7.43 (d, J=7.1 Hz, 1H), 7.39 (d, J=7.8 Hz, 2H), 6.98 (d, J=8.9 Hz, 2H), 6.16-6.22 (m, 2H), 3.41 (td, J=6.5, 2.7 Hz, 2H), 3.15-3.21 (m, 4H), 2.93 (br t, J=6.4 Hz, 2H), 2.80 (td, J=15.7, 4.3 Hz, 2H), 2.66-2.71 (m, 4H); LCMS: [M+H]⁺=500.57.

### Example 237: 6-(2-amino-6-fluoro-5-(4-(4-(2,2,2-trifluoroethyl)piperazin-1-yl)phenyl)pyridin-3-yl)-7-fluoro-3,4-dihydroisoquinolin-1(2H)-one (I-237)

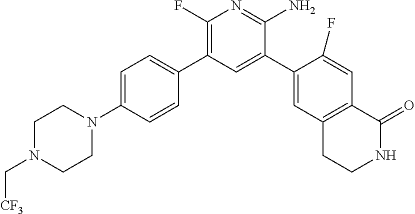 [SMILES: Nc1nc(F)c(-c2ccc(N3CCN(CC(F)(F)F)CC3)cc2)cc1-c1cc2c(cc1F)C(=O)NCC2]

A procedure similar to Example 236 using 6-(2-amino-6-fluoro-5-(4-(piperazin-1-yl)phenyl)pyridin-3-yl)-7-fluoro-3,4-dihydroisoquinolin-1(2H)-one TFA (25 mg, 0.045 mmol), N,N-diisopropylethylamine (0.079 ml, 0.455 mmol), DMF (1.0 mL) and 2,2,2-trifluoroethyl trifluoromethanesulfonate (15.84 mg, 0.068 mmol) afforded the title compound (18.2 mg, 77%) as a white solid. ¹H NMR (500 MHz, DMSO-d₆) δ 8.10 (br s, 1H), 7.60 (t, J=10.9 Hz, 2H), 7.43 (d, J=7.1 Hz, 1H), 7.39 (br d, J=8.0 Hz, 2H), 6.98 (d, J=8.9 Hz, 2H), 6.19 (s, 2H), 3.40 (br dd, J=6.5, 2.4 Hz, 2H), 3.25 (q, J=10.3 Hz, 2H), 3.15-3.21 (m, 4H), 2.93 (br t, J=6.4 Hz, 2H), 2.73-2.81 (m, 4H); LCMS: [M+H]⁺=518.62.

### Example 238: 6-(2-amino-6-fluoro-5-(4-(piperidin-4-yl)phenyl)pyridin-3-yl)-7-fluoro-3,4-dihydroisoquinolin-1(2H)-one 2,2,2-trifluoroacetate (1-238)

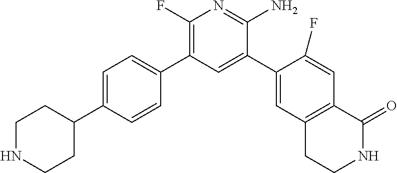 [SMILES: Nc1nc(F)c(-c2ccc(C3CCNCC3)cc2)cc1-c1cc2c(cc1F)C(=O)NCC2]

Step 1: tert-butyl 4-(4-(6-amino-2-fluoro-5-(7-fluoro-1-oxo-1,2,3,4-tetrahydroisoquinolin-6-yl)pyridin-3-yl)phenyl)piperidine-1-carboxylate

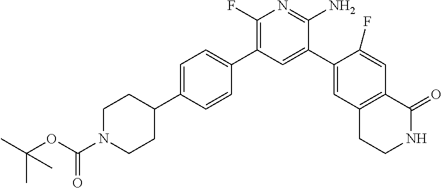 [SMILES: CC(C)(C)OC(=O)N1CCC(c2ccc(-c3cc(-c4cc5c(cc4F)C(=O)NCC5)c(N)nc3F)cc2)CC1]

To a suspension of 6-(2-amino-5-bromo-6-fluoropyridin-3-yl)-7-fluoro-3,4-dihydroisoquinolin-1(2H)-one (370 mg, 1.05 mmol), 4-(N-Boc-piperidino)phenylboronic acid pinacol ester (486 mg, 1.25 mmol) and Cs₂CO₃ (851 mg, 2.61 mmol) in 3:1 DME/H₂O (16 mL) was added PdCl₂dppf (76 mg, 0.104 mmol). The mixture was flushed with nitrogen and heated in a microwave apparatus at 90° C. for 2.5 h. The reaction was concentrated onto Celite® and purified by silica gel chromatography eluting with 0-100% EtOAc/Hexanes to give the product (498 mg, 0.932 mmol, 89%) as a brown solid. LCMS: [M+H]⁺=535.67.

Step 2: 6-(2-amino-6-fluoro-5-(4-(piperidin-4-yl)phenyl)pyridin-3-yl)-7-fluoro-3,4-dihydroisoquinolin-1(2H)-one

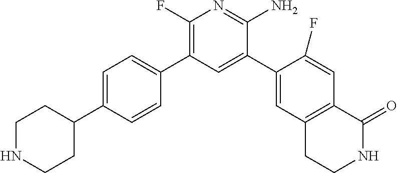 [SMILES: Nc1nc(F)c(-c2ccc(C3CCNCC3)cc2)cc1-c1cc2c(cc1F)C(=O)NCC2]

To a solution of tert-butyl 4-(4-(6-amino-2-fluoro-5-(7-fluoro-1-oxo-1,2,3,4-tetrahydroisoquinolin-6-yl)pyridin-3-yl)phenyl)piperidine-1-carboxylate (498 mg, 0.932 mmol) in CH₂Cl₂ (5.0 ml) was added TFA (2.1 mL, 27.9 mmol). The reaction was stirred at RT for 3 d. The mixture was concentrated onto Celite® and purified by reverse phase chromatography (C18, 0-100% MeCN—H₂O) to give TFA salt of the title compound (464 mg, 91%) as a beige solid. ¹H NMR (500 MHz, DMSO-d₆) δ 8.18-8.44 (m, 1H), 8.12 (br s, 1H), 7.63 (t, J=10.4 Hz, 2H), 7.51 (br d, J=7.7 Hz, 2H), 7.43 (d, J=7.1 Hz, 1H), 7.28 (d, J=8.2 Hz, 2H), 6.32 (s, 2H), 3.37-3.44 (m, 4H), 2.96-3.04 (m, 2H), 2.93 (br t, J=6.4 Hz, 2H), 2.82-2.90 (m, 1H), 1.96 (br d, J=13.2 Hz, 2H), 1.72-1.86 (m, 2H); LCMS: [M+H]⁺=435.64.

### Example 239: 6-(2-amino-6-fluoro-5-(4-(4-fluoro-1-isopropylpiperidin-4-yl)phenyl)pyridin-3-yl)-3,4-dihydroisoquinolin-1(2H)-one (I-239)

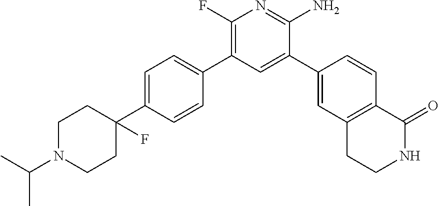 [SMILES: CC(C)N1CCC(F)(c2ccc(-c3cc(-c4ccc5c(c4)CCNC5=O)c(N)nc3F)cc2)CC1]

A mixture of 6-(2-amino-6-fluoro-5-(4-(4-fluoropiperidin-4-yl)phenyl)pyridin-3-yl)-3,4-dihydroisoquinolin-1(2H)-one (55 mg, 0.114 mmol), 2-iodopropane (77 mg, 0.456 mmol) DMF (2.0 mL) and Et₃N (0.159 ml, 1.139 mmol) was stirred overnight at RT. The reaction mixture was concentrated onto Celite®. Silica gel chromatography (eluting with CH₂Cl₂ containing 0-3% MeOH and 0-0.3% NH₄OH), followed by filtration through a Waters PoraPak CX column afforded the title compound (18 mg, 32%) as an off white powder. ¹H NMR (500 MHz, DMSO-d₆) δ 7.9-8.0 (m, 1H), 7.9-7.9 (m, 1H), 7.6-7.7 (m, 1H), 7.5-7.6 (m, 2H), 7.4-7.5 (m, 4H), 6.2-6.4 (m, 2H), 3.4-3.5 (m, 3H), 2.9-3.0 (m, 2H), 2.7-2.9 (m, 3H), 2.5-2.6 (m, 1H), 2.0-2.2 (m, 2H), 1.9-2.0 (m, 2H), 1.0-1.1 (m, 6H); LCMS: [M+H]⁺=477.63.

### Example 240: 6-(2-amino-6-fluoro-5-(4-(4-(3,3,3-trifluoropropyl)piperazin-1-yl)phenyl)pyridin-3-yl)-3,4-dihydroisoquinolin-1(2H)-one (I-240)

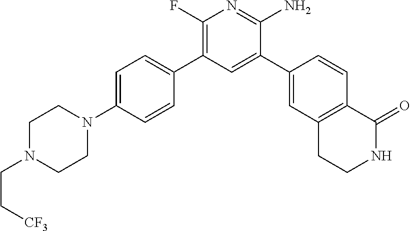 [SMILES: Nc1nc(F)c(-c2ccc(N3CCN(CCC(F)(F)F)CC3)cc2)cc1-c1ccc2c(c1)CCNC2=O]

A procedure similar to Example 236 using 6-(2-amino-6-fluoro-5-(4-(piperazin-1-yl)phenyl)pyridin-3-yl)-3,4-dihydroisoquinolin-1(2H)-one, trifluoroacetate (30 mg, 0.056 mmol) and N,N-diisopropylethylamine (0.098 ml, 0.564 mmol) in DMF (1.0 ml) and 1,1,1-trifluoro-3-iodopropane (18.96 mg, 0.085 mmol) afforded the title compound (10.6 mg, 37%) as a white solid. ¹H NMR (500 MHz, DMSO-d₆) δ 7.87 (br s, 1H), 7.83 (d, J=8.0 Hz, 1H), 7.51 (d, J=10.2 Hz, 1H), 7.39 (br d, J=8.0 Hz, 1H), 7.37 (s, 1H), 7.33 (br d, J=8.0 Hz, 2H), 6.91 (d, J=8.9 Hz, 2H), 6.10 (s, 2H), 3.33 (td, J=6.4, 2.5 Hz, 2H), 3.07-3.12 (m, 4H), 2.88 (br t, J=6.4 Hz, 2H), 2.42-2.44 (m, 8H); LCMS: [M+H]⁺=514.48.

### Example 241: 6-(2-amino-6-fluoro-5-(4-(1-(2-methoxyethyl)piperidin-4-yl)phenyl)pyridin-3-yl)-7-fluoro-3,4-dihydroisoquinolin-1(2H)-one (I-241)

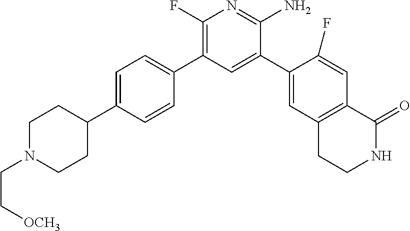 [SMILES: COCCN1CCC(c2ccc(-c3cc(-c4cc5c(cc4F)C(=O)NCC5)c(N)nc3F)cc2)CC1]

A procedure analogous to Example 236 using 6-(2-amino-6-fluoro-5-(4-(piperidin-4-yl)phenyl)pyridin-3-yl)-7-fluoro-3,4-dihydroisoquinolin-1(2H)-one, trifluoroacetate (30 mg, 0.055 mmol), N,N-diisopropylethylamine (0.095 ml, 0.547 mmol), DMF (1.0 mL) and 2-bromoethyl methyl ether (38.0 mg, 0.273 mmol) afforded the title compound (25.1 mg, 93%) as a white solid. ¹H NMR (500 MHz, DMSO-d₆) δ 8.12 (br s, 1H), 7.59-7.68 (m, 2H), 7.50 (br d, J=7.2 Hz, 2H), 7.43 (d, J=7.1 Hz, 1H), 7.30 (br d, J=7.6 Hz, 2H), 6.31 (br s, 2H), 3.63-3.74 (m, 2H), 3.40-3.44 (m, 3H), 2.93 (br t, J=6.4 Hz, 2H), 1.94 (br d, J=19.2 Hz, 4H), 1.22-1.31 (m, 3H); LCMS: [M+H]⁺=493.61.

### Example 242: 6-(2-amino-5-(4-(1-(cyclopropylmethyl)piperidin-4-yl)phenyl)-6-fluoropyridin-3-yl)-7-fluoro-3,4-dihydroisoquinolin-1(2H)-one (I-242)

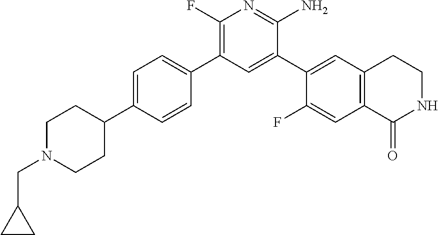 [SMILES: Nc1nc(F)c(-c2ccc(C3CCN(CC4CC4)CC3)cc2)cc1-c1cc2c(cc1F)C(=O)NCC2]

A procedure analogous to Example 236 using 6-(2-amino-6-fluoro-5-(4-(piperazin-1-yl)phenyl)pyridin-3-yl)-7-fluoro-3,4-dihydroisoquinolin-1 (2H)-one, trifluoroacetate (30 mg, 0.055 mmol) and N,N-diisopropylethylamine (0.095 mL, 0.546 mmol), DMF (1.0 ml) and 1,1,1-trifluoro-3-iodopropane (36.7 mg, 0.164 mmol) afforded the title compound (14.6 mg, 50%) as a beige solid. ¹H NMR (500 MHz, DMSO-d₆) δ 8.10 (br s, 1H), 7.60 (dd, J=13.1, 10.3 Hz, 2H), 7.43 (br d, J=7.0 Hz, 1H), 7.39 (br d, J=8.1 Hz, 2H), 6.98 (br d, J=8.8 Hz, 2H), 6.18 (s, 2H), 3.41 (br dd, J=6.1, 4.0 Hz, 2H), 3.16 (br d, J=5.0 Hz, 4H), 2.93 (br t, J=6.3 Hz, 2H), 2.57 (br d, J=4.8 Hz, 4H), 1.27 (br s, 4H); LCMS: [M+H]+=532.66.

### Example 243: 6-(2-amino-6-fluoro-5-(4-(4-(3,3,3-trifluoropropyl)piperazin-1-yl)phenyl)pyridin-3-yl)-7-fluoro-3,4-dihydroisoquinolin-1(2H)-one (I-243)

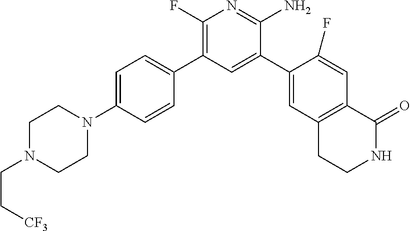 [SMILES: Nc1nc(F)c(-c2ccc(N3CCN(CCC(F)(F)F)CC3)cc2)cc1-c1cc2c(cc1F)C(=O)NCC2]

A procedure analogous to Example 236 was used, stirring 6-(2-amino-6-fluoro-5-(4-(piperazin-1-yl)phenyl)pyridin-3-yl)-7-fluoro-3,4-dihydroisoquinolin-1(2H)-one, trifluoroacetate (30 mg, 0.055 mmol), N,N-diisopropylethylamine (0.095 ml, 0.546 mmol), DMF (1.0 mL) and 1,1,1-trifluoro-3-iodopropane (36.7 mg, 0.164 mmol) for 16 h. Additional 1,1,1-trifluoro-3-iodopropane (37 mg) was added and the mixture was stirred at RT for another 16 h. Workup and purification afforded the title compound (14.6 mg, 50%) as a beige solid. ¹H NMR (500 MHz, DMSO-d₆) δ pm 8.10 (br s, 1H), 7.60 (dd, J=13.1, 10.3 Hz, 2H), 7.43 (br d, J=7.0 Hz, 1H), 7.39 (br d, J=8.1 Hz, 2H), 6.98 (br d, J=8.8 Hz, 2H), 6.18 (s, 2H), 3.41 (br dd, J=6.1, 4.0 Hz, 2H), 3.16 (br d, J=5.0 Hz, 4H), 2.93 (br t, J=6.3 Hz, 2H), 2.57 (br d, J=4.8 Hz, 4H), 1.27 (br s, 4H); LCMS: [M+H]⁺=532.66.

### Example 244: 6-(2-amino-6-fluoro-5-(4-(1-isopropylpiperidin-4-yl)phenyl)pyridin-3-yl)-7-fluoro-3,4-dihydroisoquinolin-1(2H)-one (I-244)

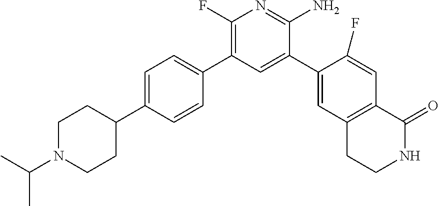 [SMILES: CC(C)N1CCC(c2ccc(-c3cc(-c4cc5c(cc4F)C(=O)NCC5)c(N)nc3F)cc2)CC1]

A procedure similar to Example 236 was used with 6-(2-amino-6-fluoro-5-(4-(piperidin-4-yl)phenyl)pyridin-3-yl)-7-fluoro-3,4-dihydroisoquinolin-1(2H)-one, trifluoroacetate (25 mg, 0.046 mmol) and N,N-diisopropylethylamine (0.079 ml, 0.456 mmol) in DMF (1.0 mL). 2-Iodopropane (38.7 mg, 0.228 mmol) was added and the mixture was stirred at RT for 16 h, then more 2-iodopropane (39 mg) was added and at the mixture was stirred at RT for 16 h. Workup and purification afforded the title compound (17.1 mg, 79%) as an off-white solid. ¹H NMR (500 MHz, DMSO-d₆) δ 8.11 (br s, 1H), 7.63 (t, J=9.9 Hz, 2H), 7.49 (br d, J=7.6 Hz, 2H), 7.43 (d, J=7.0 Hz, 1H), 7.29 (d, J=8.2 Hz, 2H), 6.30 (s, 2H), 2.93 (br t, J=6.4 Hz, 2H), 2.65-2.80 (m, 2H), 1.92 (br s, 2H), 1.70-1.86 (m, 2H), 1.11-1.27 (m, 7H); LCMS: [M+H]⁺=477.69.

### Example 245: 6-(2-amino-5-(4-(1-ethylpiperidin-4-yl)phenyl)-6-fluoropyridin-3-yl)-7-fluoro-3,4-dihydroisoquinolin-1(2H)-one (I-245)

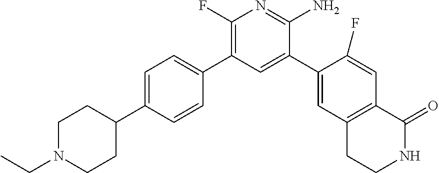 [SMILES: CCN1CCC(c2ccc(-c3cc(-c4cc5c(cc4F)C(=O)NCC5)c(N)nc3F)cc2)CC1]

To a solution of 6-(2-amino-6-fluoro-5-(4-(piperidin-4-yl)phenyl)pyridin-3-yl)-7-fluoro-3,4-dihydroisoquinolin-1(2H)-one, trifluoroacetate (25 mg, 0.046 mmol) and N,N-diisopropylethylamine (0.079 mL, 0.456 mmol) in DMF (1.0 mL) was added bromoethane (24.83 mg, 0.228 mmol). The reaction was stirred at RT over 2 d. Workup and purification in a manner similar to Example 262 afforded the title compound (17.6 mg, 0.038 mmol, 83%) as a white solid. ¹H NMR (500 MHz, DMSO-d₆) δ 8.11 (br s, 1H), 7.63 (t, J=10.3 Hz, 2H), 7.50 (br d, J=7.6 Hz, 2H), 7.43 (d, J=7.0 Hz, 1H), 7.30 (br d, J=8.2 Hz, 2H), 6.30 (s, 2H), 3.10 (br s, 1H), 2.89-2.99 (m, 4H), 2.78 (br s, 2H), 1.97 (br d, J=12.4 Hz, 2H), 1.79-1.92 (m, 2H), 1.25 (br d, J=6.5 Hz, 4H), 1.18-1.23 (m, 3H); LCMS: [M+H]⁺=463.65.

### Example 246: 6-(2-amino-6-fluoro-5-(4-(4-methylpiperazin-1-yl)phenyl)pyridin-3-yl)-7-fluoro-3,4-dihydroisoquinolin-1(2H)-one (I-246)

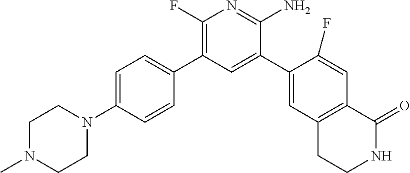 [SMILES: CN1CCN(c2ccc(-c3cc(-c4cc5c(cc4F)C(=O)NCC5)c(N)nc3F)cc2)CC1]

To a suspension of 6-(2-amino-5-bromo-6-fluoropyridin-3-yl)-7-fluoro-3,4-dihydroisoquinolin-1(2H)-one (30 mg, 0.085 mmol), 4-(4-methylpiperazin-1-yl)phenylboronic acid, pinacol ester (30.7 mg, 0.102 mmol) and Cs₂CO₃ (69.0 mg, 0.212 mmol) in a 3:1 mixture of DME (4 mL): water (1.3 mL) was added PdCl₂dppf (6.20 mg, 8.47 μmol). The mixture was flushed with nitrogen and heated in the microwave at 90° C. for 2.5 h. The reaction was concentrated onto Celite® and purified by silica gel chromatography (eluting with 0-10% MeOH/DCM+1% NH₄OH), followed by reverse phase chromatography (C18, 0-100% ACN/H₂O) to afford the title compound (28.9 mg, 76%) as a beige solid. ¹H NMR (500 MHz, DMSO-d₆) δ 8.10 (br s, 1H), 7.60 (br dd, J=13.5, 10.3 Hz, 2H), 7.43 (br d, J=6.9 Hz, 1H), 7.38 (br d, J=8.3 Hz, 2H), 6.97 (br d, J=8.4 Hz, 2H), 6.18 (br s, 2H), 3.41 (br d, J=4.2 Hz, 2H), 3.16 (br s, 4H), 2.93 (br t, J=6.1 Hz, 2H), 2.46 (br s, 4H), 2.23 (s, 3H); LCMS: [M+H]+=450.68.

### Example 247: 6-(2-amino-6-fluoro-5-(3-fluoro-4-(4-methylpiperazin-1-yl)phenyl)pyridin-3-yl)-7-fluoro-3,4-dihydroisoquinolin-1(2H)-one (I-247)

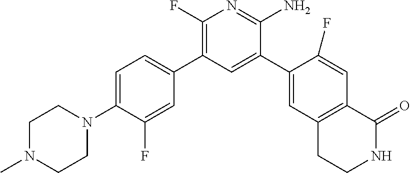 [SMILES: CN1CCN(c2ccc(-c3cc(-c4cc5c(cc4F)C(=O)NCC5)c(N)nc3F)cc2F)CC1]

A procedure analogous to Example 246 using 6-(2-amino-5-bromo-6-fluoropyridin-3-yl)-7-fluoro-3,4-dihydroisoquinolin-1(2H)-one (30 mg, 0.085 mmol), 3-fluoro-4-(4-methylpiperazino)phenylboronic acid pinacol ester (32.6 mg, 0.102 mmol) Cs₂CO₃ (69.0 mg, 0.212 mmol), DME (4 mL), water (1.3 mL) and PdCl₂dppf (6.20 mg, 8.47 μmol) afforded the title compound (28.4 mg, 72%) as an off-white solid. ¹H NMR (500 MHz, DMSO-d₆) δ 8.11 (br s, 1H), 7.66 (d, J=10.2 Hz, 1H), 7.61 (d, J=10.0 Hz, 1H), 7.43 (d, J=7.1 Hz, 1H), 7.33 (br d, J=14.6 Hz, 1H), 7.29 (br d, J=8.6 Hz, 1H), 7.05 (t, J=9.0 Hz, 1H), 6.31 (s, 2H), 3.41 (td, J=6.4, 2.6 Hz, 2H), 3.04 (br s, 4H), 2.93 (br t, J=6.4 Hz, 2H), 2.48 (br s, 4H), 2.23 (s, 3H); LCMS: [M+H]⁺=468.54.

### Example 248: 6-(2-amino-6-fluoro-5-(4-(1-(2,2,2-trifluoroethyl)piperidin-4-yl)phenyl)pyridin-3-yl)-7-fluoro-3,4-dihydroisoquinolin-1(2H)-one (I-248)

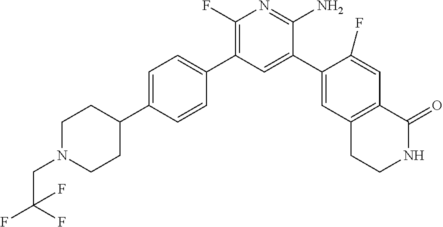 [SMILES: Nc1nc(F)c(-c2ccc(C3CCN(CC(F)(F)F)CC3)cc2)cc1-c1cc2c(cc1F)C(=O)NCC2]

A procedure analogous to Example 236 stirring 6-(2-amino-6-fluoro-5-(4-(piperidin-4-yl)phenyl)pyridin-3-yl)-7-fluoro-3,4-dihydroisoquinolin-1(2H)-one, trifluoroacetate (25 mg, 0.046 mmol), N,N-diisopropylethylamine (0.079 mL, 0.456 mmol), DMF (1.0 mL) and 2,2,2-trifluoroethyl trifluoromethanesulfonate (21.16 mg, 0.091 mmol) at RT over 2 d afforded the title compound (13.7 mg, 58%) as a white solid. ¹H NMR (500 MHz, DMSO-d₆) δ 8.03 (br s, 1H), 7.55 (t, J=10.8 Hz, 2H), 7.33-7.42 (m, 3H), 7.23 (br d, J=8.2 Hz, 2H), 6.21 (s, 2H), 3.33 (br s, 3H), 3.12 (br d, J=10.3 Hz, 2H), 2.95 (br d, J=11.3 Hz, 2H), 2.85 (br t, J=6.3 Hz, 2H), 2.34-2.40 (m, 2H), 1.64-1.71 (m, 2H), 1.55-1.64 (m, 2H); LCMS: [M+H]⁺=517.37.

### Example 249: 6-(2-amino-6-fluoro-5-(4-(4-methylpiperazin-1-yl)-3-(trifluoromethyl)phenyl)pyridin-3-yl)-7-fluoro-3,4-dihydroisoquinolin-1(2H)-one (I-249)

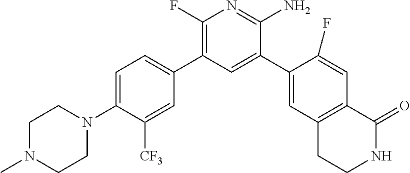 [SMILES: CN1CCN(c2ccc(-c3cc(-c4cc5c(cc4F)C(=O)NCC5)c(N)nc3F)cc2C(F)(F)F)CC1]

A procedure analogous to Example 246 using 6-(2-amino-5-bromo-6-fluoropyridin-3-yl)-7-fluoro-3,4-dihydroisoquinolin-1(2H)-one (30 mg, 0.085 mmol), 3-(trifluoromethyl)-4-(N-methylpiperazin-1-yl)phenyl boronic acid pinacol ester (37.6 mg, 0.102 mmol), Cs₂CO₃ (69.0 mg, 0.212 mmol), DME (4 mL), water (1.3 mL) and PdCl₂dppf (6.20 mg, 8.47 μmol) afforded the title compound (14.2 mg, 32%) as a white solid. ¹H NMR (500 MHz, DMSO-d₆) δ 8.04 (br s, 1H), 7.73 (br d, J=8.6 Hz, 1H), 7.70 (s, 1H), 7.67 (d, J=10.2 Hz, 1H), 7.48-7.57 (m, 2H), 7.36 (d, J=7.0 Hz, 1H), 6.34 (s, 2H), 3.34 (td, J=6.5, 2.7 Hz, 2H), 2.80-2.90 (m, 6H), 2.19 (br s, 3H); LCMS: [M+H]⁺=518.62.

### Example 250: 6-(2-amino-6-fluoro-5-(2-fluoro-5-nitro-4-((3R,5S)-3,4,5-trimethylpiperazin-1-yl)phenyl)pyridin-3-yl)-3,4-dihydroisoquinolin-1(2H)-one formate (I-250)

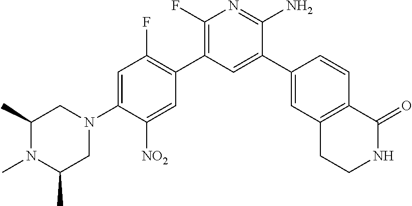 [SMILES: C[C@@H]1CN(c2cc(F)c(-c3cc(-c4ccc5c(c4)CCNC5=O)c(N)nc3F)cc2[N+](=O)[O-])C[C@H](C)N1C]

A procedure analogous to Example 246 using 6-(2-amino-5-bromo-6-fluoropyridin-3-yl)-3,4-dihydroisoquinolin-1(2H)-one (50 mg, 0.149 mmol), (2S,6R)-4-(5-fluoro-2-nitro-4-(4,4,5,5-tetramethyl-1,3,2-dioxaborolan-2-yl)phenyl)-1,2,6-trimethylpiperazine (70.2 mg, 0.178 mmol, prepared according to methods described in WO 2017/147700), Cs₂CO₃ (121 mg, 0.372 mmol), DME (4 mL), water (1.3 mL) and PdCl₂dppf (10.88 mg, 0.015 mmol) afforded the title compound (15.7 mg, 19%) as a bright yellow solid. ¹H NMR (500 MHz, DMSO-d₆) δ 8.30 (br s, 1H), 8.02 (d, J=8.0 Hz, 1H), 7.95 (br s, 1H), 7.91 (d, J=8.0 Hz, 1H), 7.60 (d, J=9.7 Hz, 1H), 7.46 (d, J=8.1 Hz, 1H), 7.44 (s, 1H), 7.20 (d, J=12.5 Hz, 1H), 6.46 (s, 2H), 3.10 (br d, J=12.1 Hz, 2H), 2.95 (br t, J=6.5 Hz, 2H), 2.69 (br t, J=11.4 Hz, 2H), 2.24-2.31 (m, 2H), 2.20 (s, 3H), 1.02 (d, J=6.1 Hz, 6H); LCMS: [M+H]+=523.63.

### Example 251: 6-(2-amino-6-fluoro-5-(4-(4-fluoro-1-(2-methoxyethyl)piperidin-4-yl)phenyl)pyridin-3-yl)-3,4-dihydroisoquinolin-1(2H)-one (I-251)

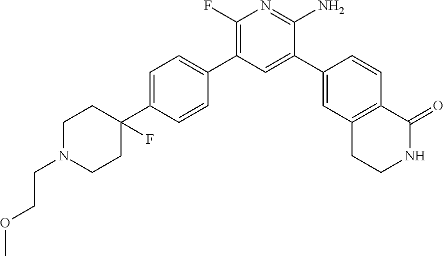 [SMILES: COCCN1CCC(F)(c2ccc(-c3cc(-c4ccc5c(c4)CCNC5=O)c(N)nc3F)cc2)CC1]

Step 1: tert-butyl 4-(4-(6-amino-2-fluoro-5-(1-oxo-1,2,3,4-tetrahydroisoquinolin-6-yl)pyridin-3-yl)phenyl)-4-fluoropiperidine-1-carboxylate

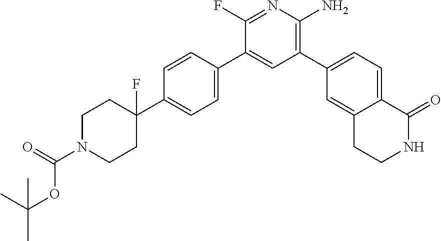 [SMILES: CC(C)(C)OC(=O)N1CCC(F)(c2ccc(-c3cc(-c4ccc5c(c4)CCNC5=O)c(N)nc3F)cc2)CC1]

A procedure analogous to that of Example 43, Step 5 with tert-butyl 4-(4-chlorophenyl)-4-fluoropiperidine-1-carboxylate (72 mg, 0.218 mmol), 6-(2-amino-6-fluoro-5-(4,4,5,5-tetramethyl-1,3,2-dioxaborolan-2-yl)pyridin-3-yl)-3,4-dihydroisoquinolin-1(2H)-one (117 mg, 0.305 mmol) afforded the product as a white solid (123 mg, 100% yield based on purity of 95%). LCMS: [M+H]⁺=535.60.

Step 2: 6-(2-Amino-6-fluoro-5-(4-(4-fluoropiperidin-4-yl)phenyl)pyridin-3-yl)-3,4-dihydroisoquinolin-1(2H)-one

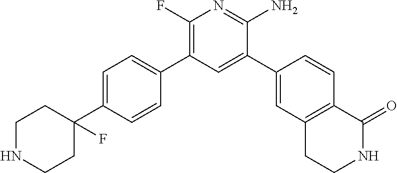 [SMILES: Nc1nc(F)c(-c2ccc(C3(F)CCNCC3)cc2)cc1-c1ccc2c(c1)CCNC2=O]

A solution 4.0 M HCl in dioxane (1.37 mL, 5.46 mmol) was added to a solution of tert-butyl 4-(4-(6-amino-2-fluoro-5-(1-oxo-1,2,3,4-tetrahydroisoquinolin-6-yl)pyridin-3-yl)phenyl)-4-fluoropiperidine-1-carboxylate (123 mg, 0.219 mmol) in MeOH (4 mL), and the reaction mixture was stirred overnight at RT. The reaction mixture was concentrated to dryness, the residue was dissolved in MeOH and passed through a Waters PoraPak CX column to afford the product as a white powder (106 mg, 100% yield based on purity of 90%). LCMS: [M+H]⁺=415.52 (-F).

Step 3: 6-(2-amino-6-fluoro-5-(4-(4-fluoro-1-(2-methoxyethyl)piperidin-4-yl)phenyl)pyridin-3-yl)-3,4-dihydroisoquinolin-1(2H)-one

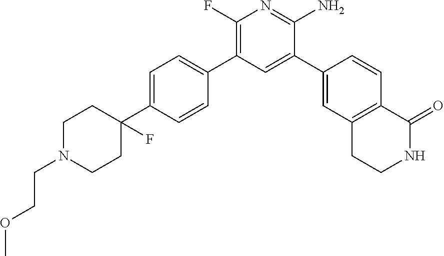 [SMILES: COCCN1CCC(F)(c2ccc(-c3cc(-c4ccc5c(c4)CCNC5=O)c(N)nc3F)cc2)CC1]

A procedure analogous to that of Example 90, Step 2, by stirring 6-(2-amino-6-fluoro-5-(4-(4-fluoropiperidin-4-yl)phenyl)pyridin-3-yl)-3,4-dihydroisoquinolin-1(2H)-one (50 mg, 0.104 mmol), and 2-bromoethyl methyl ether (0.039 mL, 0.414 mmol) overnight at RT, afforded the title compound as a yellow powder (6.5 mg, 12%). ¹H NMR (500 MHz, DMSO-d₆) δ 7.9-8.0 (m, 1H), 7.9-7.9 (m, 1H), 7.6-7.7 (m, 1H), 7.5-7.6 (m, 2H), 7.4-7.5 (m, 4H), 6.3-6.5 (m, 2H), 3.5-3.5 (m, 2H), 3.4-3.4 (m, 2H), 3.2-3.3 (m, 3H), 2.9-3.0 (m, 2H), 2.7-2.9 (m, 2H), 2.5-2.6 (m, 2H), 2.3-2.4 (m, 2H), 2.0-2.2 (m, 2H), 1.8-2.0 (m, 2H); LCMS: [M+H]⁺=493.61.

### Example 252: 6-(2-amino-5-(2-chloro-4-(4-methylpiperazin-1-yl)phenyl)-6-fluoropyridin-3-yl)-7-fluoro-3,4-dihydroisoquinolin-1(2H)-one (I-252)

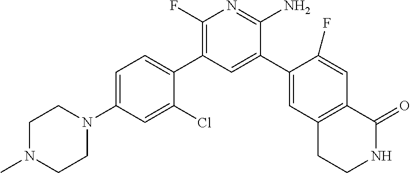 [SMILES: CN1CCN(c2ccc(-c3cc(-c4cc5c(cc4F)C(=O)NCC5)c(N)nc3F)c(Cl)c2)CC1]

A procedure analogous to Example 246 using 6-(2-amino-5-bromo-6-fluoropyridin-3-yl)-7-fluoro-3,4-dihydroisoquinolin-1(2H)-one (30 mg, 0.085 mmol), 2-chloro-4-(n-methylpiperazin-1-yl)phenylboronic acid (25.9 mg, 0.102 mmol) and Cs₂CO₃ (69.0 mg, 0.212 mmol), DME (4 mL), water (1.3 mL) and PdCl₂dppf (6.20 mg, 8.47 μmol) afforded the title compound (14.6 mg, 36%) as a beige solid. ¹H NMR (500 MHz, DMSO-d₆) δ 8.10 (br s, 1H), 7.61 (d, J=10.2 Hz, 1H), 7.36-7.43 (m, 2H), 7.23 (d, J=8.6 Hz, 1H), 7.05 (d, J=2.1 Hz, 1H), 6.96 (dd, J=8.6, 2.1 Hz, 1H), 6.28 (s, 2H), 3.30-3.37 (m, 4H), 3.23 (br s, 4H), 2.92 (br t, J=6.3 Hz, 2H), 2.27 (br s, 3H); LCMS: [M+H]⁺=484.65.

### Example 253: 6-(2-amino-6-fluoro-5-(1-methylindolin-5-yl)pyridin-3-yl)-3,4-dihydroisoquinolin-1(2H)-one (I-253)

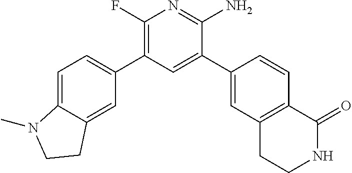 [SMILES: CN1CCc2cc(-c3cc(-c4ccc5c(c4)CCNC5=O)c(N)nc3F)ccc21]

A procedure similar to Example 246 using 6-(2-amino-5-bromo-6-fluoropyridin-3-yl)-3,4-dihydroisoquinolin-1(2H)-one (50 mg, 0.149 mmol), 1-methyl-5-(4,4,5,5-tetramethyl-1,3,2-dioxaborolan-2-yl)indoline (46.3 mg, 0.178 mmol) and Cs₂CO₃ (121 mg, 0.372 mmol), DME (4 mL), water (1.3 mL) and PdCl₂dppf (10.88 mg, 0.015 mmol) afforded the title compound (26.9 mg, 47%) as a beige solid. ¹H NMR (500 MHz, DMSO-d₆) δ 7.94 (br s, 1H), 7.91 (br d, J=7.8 Hz, 1H), 7.54 (br d, J=10.2 Hz, 1H), 7.47 (br d, J=8.2 Hz, 1H), 7.44 (s, 1H), 7.23 (br s, 1H), 7.20 (br d, J=8.0 Hz, 1H), 6.54 (br d, J=8.1 Hz, 1H), 6.11 (br s, 2H), 3.40-3.43 (m, 2H), 3.28 (br t, J=8.1 Hz, 2H), 2.96 (br t, J=6.1 Hz, 2H), 2.90 (br t, J=8.1 Hz, 2H), 2.72 (s, 3H); LCMS: [M+H]⁺=389.63.

### Example 254: 6-(2-amino-6-fluoro-5-(2-methyl-1,2,3,4-tetrahydroisoquinolin-6-yl)pyridin-3-yl)-3,4-dihydroisoquinolin-1(2H)-one (I-254)

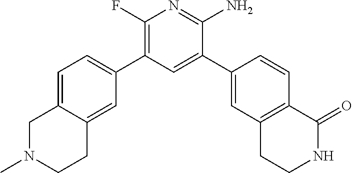 [SMILES: CN1CCc2cc(-c3cc(-c4ccc5c(c4)CCNC5=O)c(N)nc3F)ccc2C1]

A procedure similar to Example 246 using 6-(2-amino-5-bromo-6-fluoropyridin-3-yl)-3,4-dihydroisoquinolin-1(2H)-one (50 mg, 0.149 mmol), 2-methyl-1,2,3,4-tetrahydroisoquinoline-6-boronic acid pinacol ester (48.8 mg, 0.178 mmol), Cs₂CO₃ (121 mg, 0.372 mmol), DME (4 mL), water (1.3 mL), PdCl₂dppf (10.88 mg, 0.015 mmol) afforded the title compound (31.7 mg, 53%) as a beige solid. ¹H NMR (500 MHz, DMSO-d₆) δ 7.87 (br s, 1H), 7.84 (d, J=8.0 Hz, 1H), 7.55 (d, J=10.0 Hz, 1H), 7.40 (br d, J=8.1 Hz, 1H), 7.23 (br s, 1H), 7.21 (s, 1H), 7.01 (d, J=8.2 Hz, 1H), 6.19 (s, 2H), 3.42 (s, 2H), 3.31-3.37 (m, 3H), 2.88 (br t, J=6.5 Hz, 2H), 2.77 (br t, J=5.6 Hz, 2H), 2.52 (br t, J=5.8 Hz, 2H), 2.27 (s, 3H); LCMS: [M+H]⁺=403.61.

### Example 255: 6-(2-amino-6-fluoro-5-(1,2,3,4-tetrahydroisoquinolin-6-yl)pyridin-3-yl)-3,4-dihydroisoquinolin-1(2H)-one 2,2,2-trifluoroacetate (I-255)

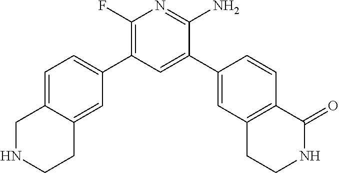 [SMILES: Nc1nc(F)c(-c2ccc3c(c2)CCNC3)cc1-c1ccc2c(c1)CCNC2=O]

Step 1: tert-butyl 6-(6-amino-2-fluoro-5-(1-oxo-1,2,3,4-tetrahydroisoquinolin-6-yl)pyridin-3-yl)-3,4-dihydroisoquinoline-2(1H)-carboxylate

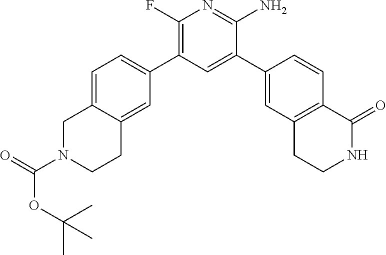 [SMILES: CC(C)(C)OC(=O)N1CCc2cc(-c3cc(-c4ccc5c(c4)CCNC5=O)c(N)nc3F)ccc2C1]

A procedure similar to Example 246 using 6-(2-amino-5-bromo-6-fluoropyridin-3-yl)-3,4-dihydroisoquinolin-1(2H)-one (230 mg, 0.684 mmol), tert-butyl 6-(4,4,5,5-tetramethyl-1,3,2-dioxaborolan-2-yl)-3,4-dihydroisoquinoline-2(1 H)-carboxylate (295 mg, 0.821 mmol), Cs₂CO₃ (557 mg, 1.71 mmol) DME (4 mL), water (1.3 mL) and PdCl₂dppf (50.1 mg, 0.068 mmol) afforded the product (294 mg, 88%) as a beige solid. LCMS: [M+H]⁺=489.60.

Step 2: 6-(2-amino-6-fluoro-5-(1,2,3,4-tetrahydroisoquinolin-6-yl)pyridin-3-yl)-3,4-dihydroisoquinolin-1(2H)-one

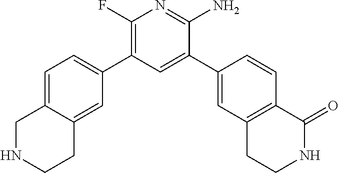 [SMILES: Nc1nc(F)c(-c2ccc3c(c2)CCNC3)cc1-c1ccc2c(c1)CCNC2=O]

To a solution of tert-butyl 6-(6-amino-2-fluoro-5-(1-oxo-1,2,3,4-tetrahydroisoquinolin-6-yl)pyridin-3-yl)-3,4-dihydroisoquinoline-2(1 H)-carboxylate (294 mg, 0.602 mmol) in CH₂Cl₂ (5.0 mL) was added TFA (1.38 mL, 18.1 mmol). The reaction was stirred at RT overnight. The mixture was concentrated onto Celite® and purified by reverse phase chromatography (C18, 0-100% MeCN—H₂O) to afford 6-(2-amino-6-fluoro-5-(1,2,3,4-tetrahydroisoquinolin-6-yl)pyridin-3-yl)-3,4-dihydroisoquinolin-1(2H)-one, trifluoroacetate salt (266 mg, 88%) as a white solid. ¹H NMR (500 MHz, DMSO-d₆) δ 8.74-8.92 (m, 1H), 7.96 (br s, 1H), 7.92 (d, J=8.0 Hz, 1H), 7.65 (d, J=10.2 Hz, 1H), 7.42-7.48 (m, 4H), 7.26 (d, J=8.0 Hz, 1H), 6.35 (s, 2H), 4.28 (s, 2H), 3.38-3.44 (m, 4H), 3.02 (br t, J=6.1 Hz, 2H), 2.96 (t, J=6.5 Hz, 2H); LCMS: [M+H]⁺=389.63.

### Example 256: 6-(2-amino-5-(4-(1-(2,2-difluoroethyl)piperidin-4-yl)phenyl)-6-fluoropyridin-3-yl)-7-fluoro-3,4-dihydroisoquinolin-1(2H)-one (I-256)

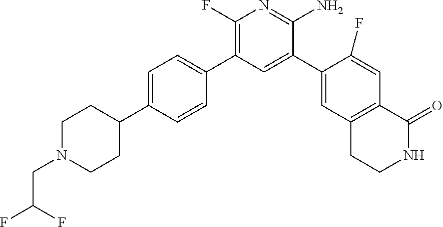 [SMILES: Nc1nc(F)c(-c2ccc(C3CCN(CC(F)F)CC3)cc2)cc1-c1cc2c(cc1F)C(=O)NCC2]

A procedure similar to Example 236 using 6-(2-amino-6-fluoro-5-(4-(piperidin-4-yl)phenyl)pyridin-3-yl)-7-fluoro-3,4-dihydroisoquinolin-1(2H)-one, trifluoroacetate (25 mg, 0.046 mmol) and N,N-diisopropylethylamine (0.079 mL, 0.456 mmol), DMF (1.0 mL) and 2,2-difluoroethyl trifluoromethanesulfonate (19.52 mg, 0.091 mmol) afforded the title compound (9.3 mg, 41%) as a white solid. ¹H NMR (500 MHz, DMSO-d₆) δ 8.03 (br s, 1H), 7.55 (t, J=10.3 Hz, 2H), 7.38 (br d, J=7.2 Hz, 2H), 7.36 (d, J=7.1 Hz, 1H), 7.22 (d, J=8.2 Hz, 2H), 6.20-6.24 (m, 2H), 3.33 (td, J=6.5, 2.7 Hz, 2H), 2.94 (br d, J=11.4 Hz, 2H), 2.85 (br t, J=6.4 Hz, 2H), 2.68 (td, J=15.7, 4.3 Hz, 2H), 2.16-2.24 (m, 2H), 1.65-1.71 (m, 2H), 1.54-1.64 (m, 2H); LCMS: [M+H]⁺=499.63.

### Example 257: 6-(2-amino-6-fluoro-5-(4-(morpholinomethyl)phenyl)pyridin-3-yl)-3,4-dihydroisoquinolin-1(2H)-one (I-257)

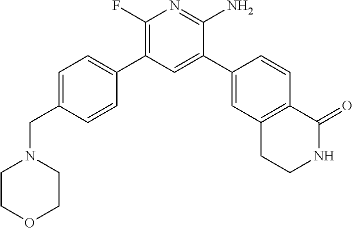 [SMILES: Nc1nc(F)c(-c2ccc(CN3CCOCC3)cc2)cc1-c1ccc2c(c1)CCNC2=O]

A procedure similar to Example 246 using 6-(2-amino-5-bromo-6-fluoropyridin-3-yl)-3,4-dihydroisoquinolin-1(2H)-one (50 mg, 0.149 mmol), 4-[4-(4,4,5,5-tetramethyl-1,3,2-dioxaborolan-2-yl)benzyl]morpholine (54.1 mg, 0.178 mmol), Cs₂CO₃ (121 mg, 0.372 mmol), DME (4 mL), water (1.3 mL), and PdCl₂dppf (10.88 mg, 0.015 mmol) afforded the title compound (29.7 mg, 46%) as an off-white solid. ¹H NMR (500 MHz, DMSO-d₆) δ 7.95 (br s, 1H), 7.91 (br d, J=8.0 Hz, 1H), 7.65 (br d, J=10.0 Hz, 1H), 7.52 (br d, J=7.6 Hz, 2H), 7.48 (br d, J=8.2 Hz, 1H), 7.46 (s, 1H), 7.35 (br d, J=8.0 Hz, 2H), 6.31 (br s, 2H), 3.58 (br s, 4H), 3.48 (s, 2H), 3.41 (br d, J=1.6 Hz, 2H), 2.96 (br t, J=6.2 Hz, 2H), 2.37 (br s, 4H); LCMS: [M+H]⁺=433.57.

### Example 258: 6-(2-amino-6-fluoro-5-(4-((4-methylpiperazin-1-yl)methyl)phenyl)pyridin-3-yl)-3,4-dihydroisoquinolin-1(2H)-one (I-258)

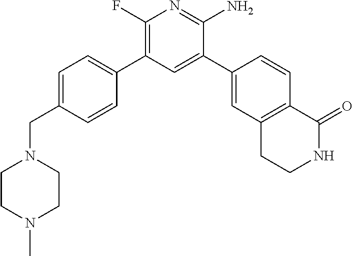 [SMILES: CN1CCN(Cc2ccc(-c3cc(-c4ccc5c(c4)CCNC5=O)c(N)nc3F)cc2)CC1]

A procedure similar to Example 246 using 6-(2-amino-5-bromo-6-fluoropyridin-3-yl)-3,4-dihydroisoquinolin-1(2H)-one (50 mg, 0.149 mmol), 4-((4-methylpiperazin-1-yl)methyl)phenylboronic acid pinacol ester (56.4 mg, 0.178 mmol), Cs₂CO₃ (121 mg, 0.372 mmol), DME (4 mL), water (1.3 mL) and PdCl₂dppf (10.88 mg, 0.015 mmol) afforded the title compound (20.2 mg, 31%) as an off-white solid. ¹H NMR (500 MHz, DMSO-d₆) δ 7.87 (br s, 1H), 7.84 (d, J=7.8 Hz, 1H), 7.57 (d, J=10.2 Hz, 1H), 7.43 (br d, J=7.5 Hz, 2H), 7.39-7.42 (m, 1H), 7.38 (s, 1H), 7.26 (d, J=8.2 Hz, 2H), 6.23 (s, 2H), 3.39 (s, 2H), 3.34 (td, J=6.5, 2.5 Hz, 2H), 2.88 (br t, J=6.5 Hz, 2H), 2.17-2.38 (m, 6H), 2.07 (s, 3H); LCMS: [M+H]⁺=446.60.

### Example 259: 6-(2-amino-6-fluoro-5-(4-(4-methylpiperazine-1-carbonyl)phenyl)pyridin-3-yl)-3,4-dihydroisoquinolin-1(2H)-one (I-259)

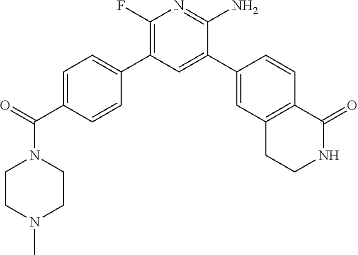 [SMILES: CN1CCN(C(=O)c2ccc(-c3cc(-c4ccc5c(c4)CCNC5=O)c(N)nc3F)cc2)CC1]

A procedure similar to Example 246 using 6-(2-amino-5-bromo-6-fluoropyridin-3-yl)-3,4-dihydroisoquinolin-1(2H)-one (50 mg, 0.149 mmol), 4-(4-methylpiperazine-1-carbonyl)phenylboronic acid, pinacol ester (58.9 mg, 0.178 mmol) and Cs₂CO₃ (121 mg, 0.372 mmol), DME (4 mL), water (1.3 mL) and PdCl₂dppf (10.88 mg, 0.015 mmol) afforded (40.6 mg, 59%) as a white solid. ¹H NMR (500 MHz, DMSO-d₆) δ 7.88 (br s, 1H), 7.84 (d, J=8.0 Hz, 1H), 7.63 (d, J=10.2 Hz, 1H), 7.56 (d, J=7.5 Hz, 2H), 7.40-7.44 (m, 1H), 7.39 (s, 1H), 7.36 (d, J=8.3 Hz, 2H), 6.33 (s, 2H), 3.54 (br s, 2H), 3.34 (td, J=6.5, 2.7 Hz, 2H), 2.88 (t, J=6.5 Hz, 2H), 2.26 (br s, 4H), 2.13 (s, 3H); LCMS: [M+H]⁺=460.64.

### Example 260: 6-(2-amino-6-fluoro-5-(4-(1-(4-methylpiperazin-1-yl)cyclopropyl)phenyl)pyridin-3-yl)-3,4-dihydroisoquinolin-1(2H)-one (I-260)

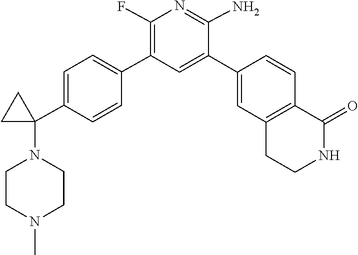 [SMILES: CN1CCN(C2(c3ccc(-c4cc(-c5ccc6c(c5)CCNC6=O)c(N)nc4F)cc3)CC2)CC1]

A procedure similar to Example 246 using 6-(2-amino-5-bromo-6-fluoropyridin-3-yl)-3,4-dihydroisoquinolin-1(2H)-one (50 mg, 0.149 mmol), 1-methyl-4-(1-[4-(4,4,5,5-tetramethyl-[1,3,2]dioxaborolan-2-yl)-phenyl]cyclopropyl)piperazine (61.1 mg, 0.178 mmol) Cs₂CO₃ (121 mg, 0.372 mmol), DME (3 mL), water (1 mL) and PdCl₂dppf (10.88 mg, 0.015 mmol) afforded the title compound (52.1 mg, 74%) as an off-white solid. ¹H NMR (500 MHz, DMSO-d₆) δ 7.87 (br s, 1H), 7.83 (d, J=8.0 Hz, 1H), 7.58 (d, J=10.2 Hz, 1H), 7.44 (br d, J=7.5 Hz, 2H), 7.40-7.42 (m, 1H), 7.39 (s, 1H), 7.22 (d, J=8.2 Hz, 2H), 6.25 (s, 2H), 3.34 (td, J=6.5, 2.6 Hz, 2H), 2.88 (br t, J=6.5 Hz, 2H), 2.37 (br s, 4H), 2.16 (br s, 4H), 1.99 (s, 3H), 0.78 (br d, J=1.7 Hz, 2H), 0.68 (br d, J=1.8 Hz, 2H); LCMS: [M+H]⁺=472.68.

### Example 261: 6-(2-amino-6-fluoro-5-(4-((1-isopropylpiperidin-4-yl)oxy)-3-(trifluoromethyl)phenyl)pyridin-3-yl)-3,4-dihydroisoquinolin-1(2H)-one (I-261)

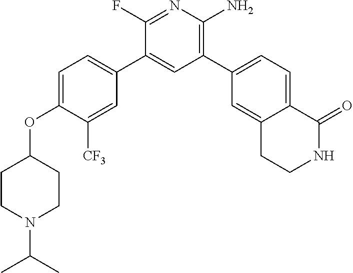 [SMILES: CC(C)N1CCC(Oc2ccc(-c3cc(-c4ccc5c(c4)CCNC5=O)c(N)nc3F)cc2C(F)(F)F)CC1]

A procedure similar to Example 281 using 6-(2-amino-6-fluoro-5-(4-(piperidin-4-yloxy)-3-(trifluoromethyl)phenyl)pyridin-3-yl)-3,4-dihydroisoquinolin-1(2H)-one (21.4 mg, 0.043 mmol) in CH₂Cl₂ (1.5 mL) and acetone (1.5 mL), 2 drops of glacial AcOH (15.41 mg, 0.257 mmol) and NaBH(OAc)₃ (54.4 mg, 0.257 mmol) afforded the title compound (23.1 mg, 95%) as a white powder. ¹H NMR (500 MHz, DMSO-d₆) δ 7.94 (br s, 1H), 7.90 (d, J=7.8 Hz, 1H), 7.76 (br d, J=8.9 Hz, 1H), 7.73 (s, 1H), 7.68 (d, J=10.1 Hz, 1H), 7.47 (d, J=7.9 Hz, 1H), 7.45 (s, 1H), 7.34 (d, J=8.9 Hz, 1H), 6.33 (s, 2H), 4.70-4.63 (m, 1H), 3.43-3.40 (m, 4H), 2.99-2.92 (m, 2H), 2.74-2.67 (m, 1H), 2.67-2.61 (m, 2H), 2.41-2.34 (m, 2H), 1.95-1.90 (m, 2H), 1.73-1.63 (m, 2H), 0.96 (d, J=6.6 Hz, 6H); LCMS: [M+H]⁺=543.65.

### Example 262: 6-(2-amino-6-fluoro-5-(4-((1-(2-methoxyethyl)piperidin-4-yl)oxy)-3-(trifluoromethyl)phenyl)pyridin-3-yl)-3,4-dihydroisoquinolin-1(2H)-one (I-262)

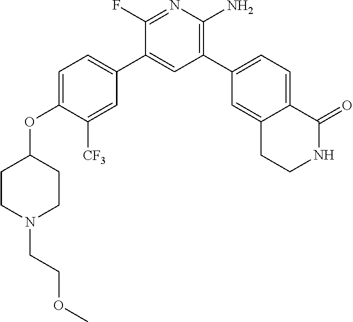 [SMILES: COCCN1CCC(Oc2ccc(-c3cc(-c4ccc5c(c4)CCNC5=O)c(N)nc3F)cc2C(F)(F)F)CC1]

A procedure similar to Example 186 using 6-(2-amino-6-fluoro-5-(4-(piperidin-4-yloxy)-3-(trifluoromethyl)phenyl)pyridin-3-yl)-3,4-dihydroisoquinolin-1(2H)-one (24 mg, 0.048 mmol) and N,N-diisopropylethylamine (0.084 ml, 0.480 mmol) in DMF (1 mL) with 2-bromoethyl methyl ether (0.014 mL, 0.144 mmol) afforded the title compound (21.1 mg, 75%) as a white powder. ¹H NMR (500 MHz, DMSO-d₆) δ 7.94 (br s, 1H), 7.91 (d, J=7.9 Hz, 1H), 7.77 (br d, J=8.8 Hz, 1H), 7.74 (s, 1H), 7.68 (d, J=10.3 Hz, 1H), 7.47 (d, J=7.9 Hz, 1H), 7.45 (s, 1H), 7.36 (br d, J=8.8 Hz, 1H), 6.34 (s, 2H), 4.81-4.64 (m, 1H), 3.54-3.44 (m, 2H), 3.40 (dt, J=2.6, 6.5 Hz, 3H), 3.25 (s, 3H), 2.95 (br t, J=6.5 Hz, 2H), 2.87-2.59 (m, 3H), 2.46-2.22 (m, 1H), 2.07-1.92 (m, 2H), 1.86-1.65 (m, 2H), 1.33-1.15 (m, 1H); LCMS: [M +H]⁺=559.67.

### Example 263: 6-(2-amino-6-fluoro-5-(4-(4-isopropylpiperazin-1-yl)-3-(trifluoromethyl)phenyl)pyridin-3-yl)-7-fluoro-3,4-dihydroisoquinolin-1(2H)-one (I-263)

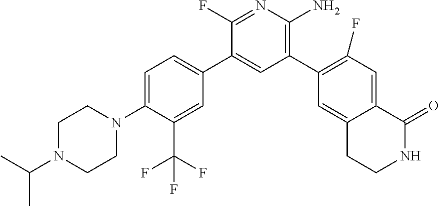 [SMILES: CC(C)N1CCN(c2ccc(-c3cc(-c4cc5c(cc4F)C(=O)NCC5)c(N)nc3F)cc2C(F)(F)F)CC1]

A procedure similar to Example 218 using 6-(2-amino-6-fluoro-5-(4-(piperazin-1-yl)-3-(trifluoromethyl)phenyl)pyridin-3-yl)-7-fluoro-3,4-dihydroisoquinolin-1(2H)-one (21.3 mg, 0.042 mmol) in CH₂Cl₂ (1.5 mL) and acetone (1.5 mL) and 2 drops glacial AcOH (15.24 mg, 0.254 mmol) and NaBH(OAc)₃ (53.8 mg, 0.254 mmol) afforded the title compound (24.1 mg, 99%) as a beige powder. ¹H NMR (500 MHz, DMSO-d₆) δ 8.12 (br s, 1H), 7.84 (br d, J=7.1 Hz, 1H), 7.80 (br s, 1H), 7.75 (d, J=10.1 Hz, 1H), 7.61 (d, J=10.1 Hz, 1H), 7.57 (d, J=8.4 Hz, 1H), 7.43 (d, J=7.0 Hz, 1H), 6.43 (br s, 2H), 3.44-3.38 (m, 4H), 3.17-2.95 (m, 4H), 2.95-2.89 (m, 3H), 1.91 (s, 2H), 1.37-1.03 (m, 6H), 1.53-1.42 (m, 2H); LCMS: [M+H]⁺=546.58.

### Example 264: 6-(2-amino-6-fluoro-5-(4-(4-(2-methoxyethyl)piperazin-1-yl)-3-(trifluoromethyl)phenyl)pyridin-3-yl)-7-fluoro-3,4-dihydroisoquinolin-1(2H)-one (I-264)

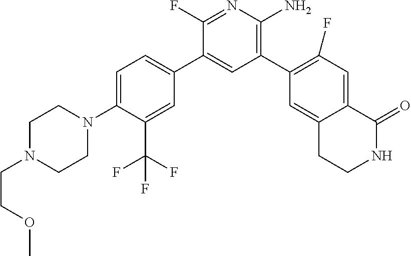 [SMILES: COCCN1CCN(c2ccc(-c3cc(-c4cc5c(cc4F)C(=O)NCC5)c(N)nc3F)cc2C(F)(F)F)CC1]

A procedure similar to Example 186 using 6-(2-amino-6-fluoro-5-(4-(piperazin-1-yl)-3-(trifluoromethyl)phenyl)pyridin-3-yl)-7-fluoro-3,4-dihydroisoquinolin-1(2H)-one (20.6 mg, 0.041 mmol) and N,N-diisopropylethylamine (0.071 ml, 0.409 mmol) in DMF (1 mL) with 2-bromoethyl methyl ether (0.012 ml, 0.123 mmol) afforded the title compound (18.4 mg, 76%) as a white powder. ¹H NMR (500 MHz, DMSO-d₆) δ 8.11 (br s, 1H), 7.79 (br d, J=8.6 Hz, 1H), 7.76 (s, 1H), 7.73 (d, J=10.3 Hz, 1H), 7.59 (dd, J=13.3, 9.2 Hz, 2H), 7.43 (d, J=7.0 Hz, 1H), 6.40 (s, 2H), 3.46 (s, 2H), 3.43-3.38 (m, 3H), 3.25 (s, 3H), 2.96-2.84 (m, 6H), 2.62-2.52 (m, 4H); LCMS: [M+H]⁺=562.53.

### Example 265: 6-(2-amino-6-fluoro-5-(3-(piperazin-1-ylmethyl)phenyl)pyridin-3-yl)-3,4-dihydroisoquinolin-1(2H)-one formate (I-265)

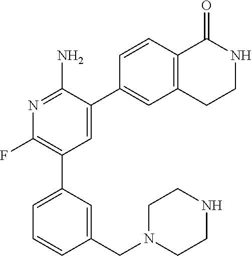 [SMILES: Nc1nc(F)c(-c2cccc(CN3CCNCC3)c2)cc1-c1ccc2c(c1)CCNC2=O]

Step 1: tert-butyl 4-(3-(6-amino-2-fluoro-5-(1-oxo-1,2,3,4-tetrahydroisoquinolin-6-yl)pyridin-3-yl)benzyl)piperazine-1-carboxylate

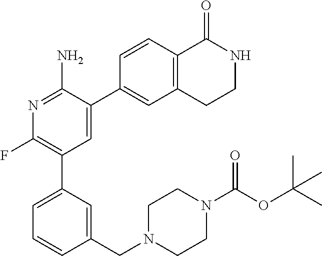 [SMILES: CC(C)(C)OC(=O)N1CCN(Cc2cccc(-c3cc(-c4ccc5c(c4)CCNC5=O)c(N)nc3F)c2)CC1]

A procedure similar to Example 246 using 6-(2-amino-5-bromo-6-fluoropyridin-3-yl)-3,4-dihydroisoquinolin-1(2H)-one (250 mg, 0.744 mmol), tert-butyl 4-(3-(4,4,5,5-tetramethyl-1,3,2-dioxaborolan-2-yl)benzyl)piperazine-1-carboxylate (359 mg, 0.892 mmol) and Cs₂CO₃ (606 mg, 1.86 mmol), DME (4 mL), water (1.3 mL), PdCl₂dppf (54.4 mg, 0.074 mmol) afforded the title compound (336 mg, 85%) as a beige solid. LCMS: [M+H]+=532.72.

Step 2: 6-(2-amino-6-fluoro-5-(3-(piperazin-1-ylmethyl)phenyl)pyridin-3-yl)-3,4-dihydroisoquinolin-1(2H)-one

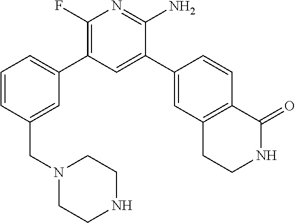 [SMILES: Nc1nc(F)c(-c2cccc(CN3CCNCC3)c2)cc1-c1ccc2c(c1)CCNC2=O]

A solution of tert-butyl 4-(3-(6-amino-2-fluoro-5-(1-oxo-1,2,3,4-tetrahydroisoquinolin-6-yl)pyridin-3-yl)benzyl)piperazine-1-carboxylate (336 mg, 0.632 mmol) in CH₂Cl₂ (3.0 mL) was added TFA (1.45 mL, 19.0 mmol). The reaction was stirred at RT overnight. The mixture was concentrated onto Celite® and purified by reverse phase chromatography (C18, 0-100% MeCN—H2O) to give the trifluoroacetate salt (287 mg, 0.526 mmol, 83% yield) as a white solid. A portion (25 mg) of the material was further purified by Waters prep HPLC (XBridge Prep C_{18 5} μm, 10×100 mm column, 95-80% 0.1% formic acid in water/0.1% formic acid in acetonitrile) to afford the title compound (20.2 mg). ¹H NMR (500 MHz, DMSO-d₆) δ 8.30 (br s, 1H), 7.96 (br s, 1H), 7.92 (d, J=7.8 Hz, 1H), 7.64 (d, J=10.2 Hz, 1H), 7.43-7.51 (m, 4H), 7.38 (t, J=7.6 Hz, 1H), 7.26 (d, J=7.6 Hz, 1H), 6.33 (s, 2H), 3.53 (s, 2H), 3.41-3.43 (m, 2H), 2.96 (br t, J=6.5 Hz, 2H), 2.88 (br s, 4H), 2.44 (br s, 4H); LCMS: [M+H]⁺=432.63.

### Example 266: 6-(2-amino-6-fluoro-5-(2-(2-methoxyethyl)-1,2,3,4-tetrahydroisoquinolin-6-yl)pyridin-3-yl)-3,4-dihydroisoquinolin-1(2H)-one (I-266)

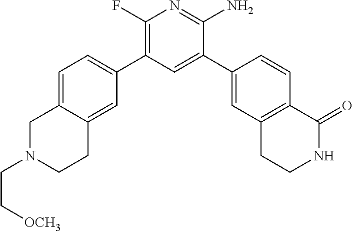 [SMILES: COCCN1CCc2cc(-c3cc(-c4ccc5c(c4)CCNC5=O)c(N)nc3F)ccc2C1]

A procedure similar to Example 236 stirring 6-(2-amino-6-fluoro-5-(1,2,3,4-tetrahydroisoquinolin-6-yl)pyridin-3-yl)-3,4-dihydroisoquinolin-1(2H)-one, trifluoroacetate (25 mg, 0.050 mmol), N,N-diisopropylethylamine (0.087 ml, 0.498 mmol), DMF (1.0 mL) and 2-bromoethyl methyl ether (34.6 mg, 0.249 mmol) The reaction was stirred at RT over 72 h afforded the title compound (7.2 mg, 32%) as a yellow solid. ¹H NMR (500 MHz, DMSO-d₆) b 7.87 (br s, 1H), 7.84 (d, J=8.0 Hz, 1H), 7.54 (d, J=10.2 Hz, 1H), 7.40 (br d, J=8.0 Hz, 1H), 7.37 (s, 1H), 7.19-7.24 (m, 2H), 7.01 (d, J=8.0 Hz, 1H), 6.20 (s, 2H), 3.54 (s, 2H), 3.46 (t, J=5.8 Hz, 2H), 3.32-3.34 (m, 2H), 3.20 (s, 3H), 2.88 (br t, J=6.4 Hz, 2H), 2.76 (br t, J=5.5 Hz, 2H), 2.62-2.67 (m, 2H), 2.58 (t, J=5.9 Hz, 2H); LCMS: [M+H]⁺=447.61.

### Example 267: 6-(2-amino-6-fluoro-5-(4-((1-isopropylpiperidin-4-yl)oxy)phenyl)pyridin-3-yl)-3,4-dihydroisoquinolin-1(2H)-one (I-267)

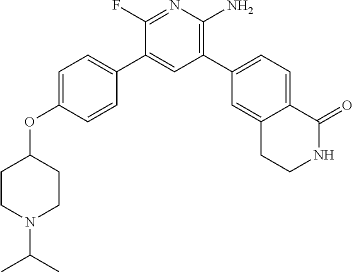 [SMILES: CC(C)N1CCC(Oc2ccc(-c3cc(-c4ccc5c(c4)CCNC5=O)c(N)nc3F)cc2)CC1]

A procedure similar to Example 218 using 6-(2-amino-6-fluoro-5-(4-(piperidin-4-yloxy)phenyl)pyridin-3-yl)-3,4-dihydroisoquinolin-1(2H)-one (23 mg, 0.053 mmol) in CH₂Cl₂ (1.5 mL), acetone (1.5 mL), 2 drops of glacial AcOH (19.16 mg, 0.319 mmol) and NaBH(OAc)₃ (67.6 mg, 0.319 mmol) afforded the title compound (20.2 mg, 76%) as a white powder. ¹H NMR (500 MHz, DMSO-d₆) δ 7.94 (br s, 1H), 7.90 (d, J=7.8 Hz, 1H), 7.60 (d, J=10.3 Hz, 1H), 7.51-7.42 (m, 4H), 7.03 (br d, J=8.2 Hz, 2H), 6.23 (s, 2H), 4.72-4.42 (m, 1H), 3.45-3.38 (m, 6H), 3.14-3.00 (m, 2H), 2.95 (br t, J=6.5 Hz, 3H), 2.11 (br d, J=3.9 Hz, 2H), 1.87-1.70 (m, 1H), 1.33-1.04 (m, 6H); LCMS: [M+H]⁺=475.63.

### Example 268: 6-(2-amino-6-fluoro-5-(3-fluoro-4-((1-isopropylpiperidin-4-yl)oxy)phenyl)pyridin-3-yl)-3,4-dihydroisoquinolin-1(2H)-one(-268)

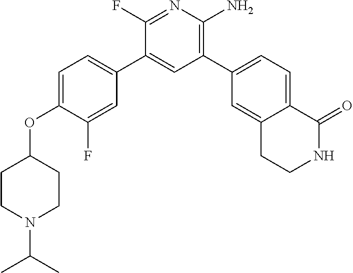 [SMILES: CC(C)N1CCC(Oc2ccc(-c3cc(-c4ccc5c(c4)CCNC5=O)c(N)nc3F)cc2F)CC1]

A procedure similar to Example 218 using 6-(2-amino-6-fluoro-5-(3-fluoro-4-(piperidin-4-yloxy)phenyl)pyridin-3-yl)-3,4-dihydroisoquinolin-1(2H)-one (22.7 mg, 0.050 mmol) in CH₂Cl₂ (1.5 mL) and acetone (1.5 mL), 2 drops of AcOH (18.16 mg, 0.302 mmol) and NaBH(OAc)₃ (64.1 mg, 0.302 mmol) afforded the title compound (17.1 mg, 66%) as a white powder. ¹H NMR (500 MHz, DMSO-d₆) δ 7.95 (br s, 1H), 7.90 (d, J=7.9 Hz, 1H), 7.66 (d, J=10.1 Hz, 1H), 7.51-7.43 (m, 3H), 7.35-7.31 (m, 1H), 7.28 (br d, J=8.2 Hz, 1H), 6.33 (s, 2H), 4.70-4.44 (m, 1H), 3.40 (dt, J=6.5, 2.7 Hz, 3H), 3.12-2.99 (m, 1H), 2.95 (br t, J=6.5 Hz, 2H), 2.25-2.05 (m, 2H), 1.88-1.72 (m, 1H), 1.30-1.04 (m, 6H), 4H under the water peak; LCMS: [M+H]⁺=493.54.

### Example 269: 6-(2-amino-6-fluoro-5-(3-fluoro-4-((1-(2-methoxyethyl)piperidin-4-yl)oxy)phenyl)pyridin-3-yl)-3,4-dihydroisoquinolin-1(2H)-one (I-269)

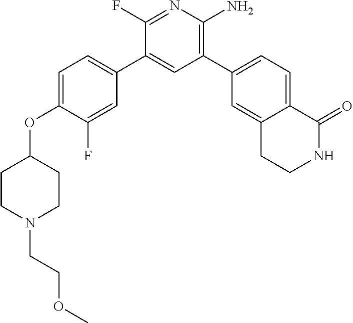 [SMILES: COCCN1CCC(Oc2ccc(-c3cc(-c4ccc5c(c4)CCNC5=O)c(N)nc3F)cc2F)CC1]

A procedure similar to Example 186 using 6-(2-amino-6-fluoro-5-(3-fluoro-4-(piperidin-4-yloxy)phenyl)pyridin-3-yl)-3,4-dihydroisoquinolin-1(2H)-one (22 mg, 0.049 mmol) and N,N-diisopropylethylamine (0.085 ml, 0.488 mmol) in DMF (1 mL) was added 2-bromoethyl methyl ether (0.014 mL, 0.147 mmol) afforded the title compound (20.1 mg, 77%) as a white powder. ¹H NMR (500 MHz, DMSO-d₆) δ 7.95 (br s, 1H), 7.91 (d, J=7.9 Hz, 1H), 7.66 (d, J=10.1 Hz, 1H), 7.52-7.42 (m, 3H), 7.36-7.30 (m, 1H), 7.29-7.21 (m, 1H), 6.33 (s, 2H), 4.46 (br s, 1H), 3.56-3.45 (m, 2H), 3.43-3.39 (m, 2H), 3.26 (s, 3H), 2.96 (s, 2H), 2.92-2.70 (m, 2H), 2.46-2.21 (m, 2H), 2.05-1.92 (m, 2H), 1.81-1.63 (m, 2H), 1.29-1.21 (m, 2H); LCMS: [M+H]⁺=509.56.

### Example 270: 6-(2-amino-5-(2,3-difluoro-4-((1-isopropylpiperidin-4-yl)oxy)phenyl)-6-fluoropyridin-3-yl)-3,4-dihydroisoquinolin-1(2H)-one (I-270)

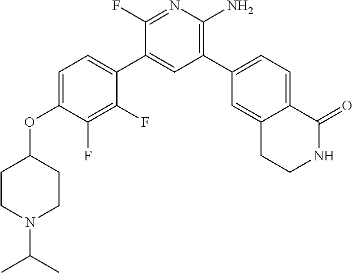 [SMILES: CC(C)N1CCC(Oc2ccc(-c3cc(-c4ccc5c(c4)CCNC5=O)c(N)nc3F)c(F)c2F)CC1]

A procedure similar to Example 218 using 6-(2-amino-5-(2,3-difluoro-4-(piperidin-4-yloxy)phenyl)-6-fluoropyridin-3-yl)-3,4-dihydroisoquinolin-1(2H)-one (20.7 mg, 0.044 mmol) in CH₂Cl₂ (1.5 mL) and acetone (1.5 mL), 2 drops of AcOH (15.92 mg, 0.265 mmol) and NaBH(OAc)₃ (56.2 mg, 0.265 mmol) afforded the title compound (11.8 mg, 50%) as a white powder. ¹H NMR (500 MHz, DMSO-d6) δ 7.95 (br s, 1H), 7.91 (d, J=7.9 Hz, 1H), 7.54 (d, J=9.5 Hz, 1H), 7.48-7.41 (m, 2H), 7.25-7.18 (m, 1H), 7.16-7.10 (m, 1H), 6.43 (s, 2H), 4.52-4.43 (m, 1H), 3.42-3.39 (m, 2H), 3.00-2.92 (m, 2H), 2.76-2.65 (m, 2H), 2.36-2.30 (m, 2H), 2.02-1.93 (m, 2H), 1.70-1.60 (m, 2H), 0.98 (s, 3H), 0.97 (s, 3H); LCMS: [M +H]⁺=511.60.

### Example 271: 6-(2-amino-5-(2,5-difluoro-4-((1-isopropylpiperidin-4-yl)oxy)phenyl)-6-fluoropyridin-3-yl)-3,4-dihydroisoquinolin-1(2H)-one (I-271)

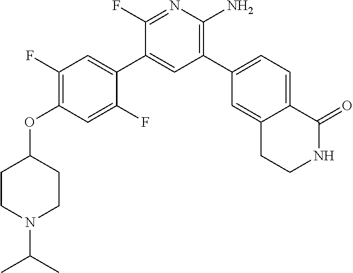 [SMILES: CC(C)N1CCC(Oc2cc(F)c(-c3cc(-c4ccc5c(c4)CCNC5=O)c(N)nc3F)cc2F)CC1]

A procedure similar to Example 218 using 6-(2-amino-5-(2,5-difluoro-4-(piperidin-4-yloxy)phenyl)-6-fluoropyridin-3-yl)-3,4-dihydroisoquinolin-1(2H)-one (23.8 mg, 0.051 mmol) in CH₂Cl₂ (1.5 mL) and acetone (1.5 mL) was added 2 drops of glacial AcOH (18.30 mg, 0.305 mmol) and NaBH(OAc)₃ (64.6 mg, 0.305 mmol) afforded the title compound (24.4 mg, 89%) as a white powder. ¹H NMR (500 MHz, DMSO-d₆) δ 7.94 (br s, 1H), 7.90 (d, J=7.9 Hz, 1H), 7.53 (d, J=9.7 Hz, 1H), 7.45 (dd, J=1.3, 7.9 Hz, 1H), 7.43 (s, 1H), 7.40 (dd, J=11.7, 7.3 Hz, 1H), 7.27 (dd, J=11.6, 7.5 Hz, 1H), 6.39 (s, 2H), 4.53-4.43 (m, 1H), 3.39 (br dd, J=6.7, 2.6 Hz, 2H), 2.94 (t, J=6.5 Hz, 2H), 2.83-2.68 (m, 2H), 2.45-2.32 (m, 2H), 2.02-1.94 (m, 2H), 1.71-1.59 (m, 2H), 1.00 (s, 3H), 0.98 (s, 3H); LCMS: [M+H]⁺=511.60.

### Example 272: 6-(2-amino-6-fluoro-5-(4-((1-isopropylpiperidin-4-yl)oxy)-2-methoxyphenyl)pyridin-3-yl)-3,4-dihydroisoquinolin-1(2H)-one (I-272)

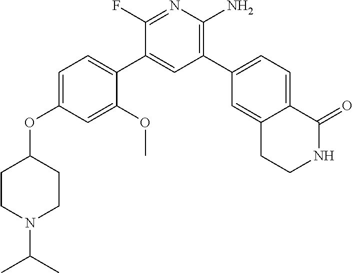 [SMILES: COc1cc(OC2CCN(C(C)C)CC2)ccc1-c1cc(-c2ccc3c(c2)CCNC3=O)c(N)nc1F]

A procedure similar to Example 218 using 6-(2-amino-6-fluoro-5-(2-methoxy-4-(piperidin-4-yloxy)phenyl)pyridin-3-yl)-3,4-dihydroisoquinolin-1(2H)-one (21.3 mg, 0.046 mmol) in CH₂Cl₂ (1.5 ml) and acetone (1.5 ml), 2 drops glacial AcOH(16.59 mg, 0.276 mmol) and NaBH(OAc)₃ (58.6 mg, 0.276 mmol) afforded the title compound (14.1 mg, 61%) as a white powder. ¹H NMR (500 MHz, DMSO-d₆) δ 7.93 (brs, 1H), 7.89 (d, J=7.9 Hz, 1H), 7.43 (dd, J=7.9, 1.5 Hz, 1H), 7.41 (s, 1H), 7.39 (d, J=9.7 Hz, 1H), 7.14 (d, J=8.3 Hz, 1H), 6.61 (d, J=2.2 Hz, 1H), 6.59 (dd, J=8.4, 2.3 Hz, 1H), 6.14 (s, 2H), 4.42-4.35 (m, 1H), 3.73 (s, 3H), 3.42-3.38 (m, 2H), 2.94 (t, J=6.5 Hz, 2H), 2.72 (br d, J=6.5 Hz, 3H), 2.38-2.29 (m, 2H), 2.01-1.93 (m, 2H), 1.64-1.55 (m, 2H), 0.99 (s, 3H), 0.98 (s, 3H); LCMS: [M+H]⁺=505.58.

### Example 273: 6-(2-amino-6-fluoro-5-(4-((1-isopropylpiperidin-4-yl)oxy)-3-methoxyphenyl)pyridin-3-yl)-3,4-dihydroisoquinolin-1(2H)-one (I-273)

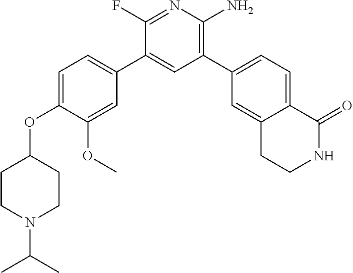 [SMILES: COc1cc(-c2cc(-c3ccc4c(c3)CCNC4=O)c(N)nc2F)ccc1OC1CCN(C(C)C)CC1]

A procedure similar to Example 218 using 6-(2-amino-6-fluoro-5-(3-methoxy-4-(piperidin-4-yloxy)phenyl)pyridin-3-yl)-3,4-dihydroisoquinolin-1(2H)-one (23.2 mg, 0.050 mmol) in CH₂Cl₂ (1.5 mL) and acetone (1.5 ml), glacial AcOH (18.07 mg, 0.301 mmol) and NaBH(OAc)₃ (63.8 mg, 0.301 mmol) afforded the title compound (17.2 mg, 65%) as a white powder. ¹H NMR (500 MHz, DMSO-d₆) δ 7.94 (br s, 1H), 7.90 (d, J=7.8 Hz, 1H), 7.64 (d, J=10.1 Hz, 1H), 7.46 (dd, J=7.9, 1.6 Hz, 1H), 7.44 (s, 1H), 7.10 (s, 1H), 7.05-6.99 (m, 2H), 6.23 (s, 2H), 4.28-4.20 (m, 1H), 3.79 (s, 3H), 3.40 (br dd, J=6.5, 2.5 Hz, 2H), 2.95 (t, J=6.5 Hz, 2H), 2.77-2.66 (m, 3H), 2.31-2.23 (m, 2H), 1.94-1.90 (m, 2H), 1.65-1.53 (m, 2H), 0.97 (s, 3H), 0.96 (s, 3H); LCMS: [M+H]⁺=505.58.

### Example 274: (S)-6-(2-amino-6-fluoro-5-(4-((1-isopropylpiperidin-3-yl)oxy)phenyl)pyridin-3-yl)-3,4-dihydroisoquinolin-1(2H)-one (I-274)

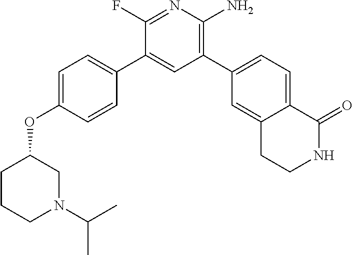 [SMILES: CC(C)N1CCC[C@H](Oc2ccc(-c3cc(-c4ccc5c(c4)CCNC5=O)c(N)nc3F)cc2)C1]

A procedure similar to Example 218 using (S)-6-(2-amino-6-fluoro-5-(4-(piperidin-3-yloxy)phenyl)pyridin-3-yl)-3,4-dihydroisoquinolin-1(2H)-one (16.5 mg, 0.038 mmol) in CH₂Cl₂ (1.5 mL) and acetone (1.5 mL), 2 drops glacial AcOH (13.75 mg, 0.229 mmol) and NaBH(OAc)₃ (48.5 mg, 0.229 mmol) afforded the title compound (14.9 mg, 78%) as a white powder. ¹H NMR (500 MHz, DMSO-d₆) δ 7.94 (br s, 1H), 7.90 (d, J=7.8 Hz, 1H), 7.60 (d, J=10.3 Hz, 1H), 7.51-7.43 (m, 4H), 7.00 (br s, 2H), 6.23 (s, 2H), 4.49-4.30 (m, 1H), 3.44-3.38 (m, 2H), 3.14-2.99 (m, 1H), 2.99-2.93 (m, 2H), 2.85-2.61 (m, 2H), 2.33-2.12 (m, 1H), 2.11-1.98 (m, 1H), 1.82-1.68 (m, 1H), 1.66-1.46 (m, 1H), 1.43-1.29 (m, 1H), 0.99 (br s, 6H); LCMS: [M+H]⁺=475.56.

### Example 275: 6-(2-amino-6-fluoro-5-(7-((1-isopropylpiperidin-4-yl)oxy)benzo[d][1,3]dioxol-4-yl)pyridin-3-yl)-3,4-dihydroisoquinolin-1(2H)-one (I-275)

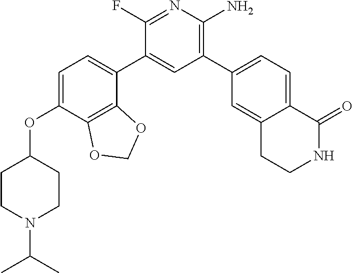 [SMILES: CC(C)N1CCC(Oc2ccc(-c3cc(-c4ccc5c(c4)CCNC5=O)c(N)nc3F)c3c2OCO3)CC1]

A procedure similar to Example 218 using 6-(2-amino-6-fluoro-5-(7-(piperidin-4-yloxy)benzo[d][1,3]dioxol-4-yl)pyridin-3-yl)-3,4-dihydroisoquinolin-1(2H)-one (22.5 mg, 0.047 mmol) in CH₂Cl₂ (1.5 ml) and acetone (1.5 mL), 2 drops of glacial AcOH (17.01 mg, 0.283 mmol) and NaBH(OAc)₃ (60.0 mg, 0.283 mmol) afforded the title compound (13.7 mg, 53%) as a white powder ¹H NMR (500 MHz, DMSO-d₆) δ 7.94 (br s, 1H), 7.90 (d, J=7.9 Hz, 1H), 7.58 (d, J=9.8 Hz, 1H), 7.45-7.38 (m, 2H), 6.92-6.84 (m, 1H), 6.68 (d, J=8.8 Hz, 1H), 6.30 (s, 2H), 5.99 (s, 2H), 4.42-4.34 (m, 1H), 3.41-3.38 (m, 2H), 2.94 (s, 2H), 2.75-2.65 (m, 3H), 2.34-2.23 (m, 2H), 1.96-1.90 (m, 2H), 1.64-1.54 (m, 2H), 0.97 (s, 3H), 0.96 (s, 3H); LCMS: [M+H]⁺=519.57.

### Example 276: 6-(6-amino-2-fluoro-5-(1-oxo-1,2,3,4-tetrahydroisoquinolin-6-yl)pyridin-3-yl)-3-((methylamino)methyl)-3,4-dihydroisoquinolin-1(2H)-one (I-276)

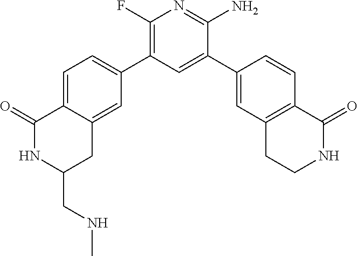 [SMILES: CNCC1Cc2cc(-c3cc(-c4ccc5c(c4)CCNC5=O)c(N)nc3F)ccc2C(=O)N1]

Step 1: 3-(3-bromophenyl)-1-methoxy-1-oxopropan-2-aminium chloride:

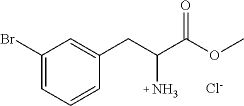

To a stirred solution of 2-(3-bromophenyl)-1-carboxyethan-1-aminium chloride (10 g, 35.7 mmol) in MeOH (300 mL) was added dropwise SOCl2 (7.7 mL, 107.3 mmol) at 0° C. The reaction mixture was then heated at 100° C. for 16 h before cooling to RT. The solvent was removed under reduced pressure to afford the product (10.4 g, quantitative yield) as an off-white solid that was used in the next step without further purification. LCMS: [M+H]⁺=258.09.

Step 2: methyl 3-(3-bromophenyl)-2-(((trichloromethoxy)carbonyl)amino)propanoate:

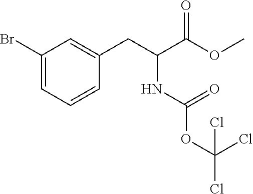 [SMILES: COC(=O)C(Cc1cccc(Br)c1)NC(=O)OC(Cl)(Cl)Cl]

To a stirred solution of 3-(3-bromophenyl)-1-methoxy-1-oxopropan-2-aminium chloride (10.5 g, 35.7 mmol) in 1, 4-dioxane (115 mL) was added trichloromethylchloroformate (5.9 mL, 50 mmol) at RT. The mixture was then heated at 100° C. for 16 h before cooling to RT. The solvent was concentrated under reduced pressure to afford crude product (14.8 g, quant) as a pale brown gummy liquid, which was used in the next step without purification. TLC: 10% MeOH: DCM; R_{f}=0.7.

Step 3: methyl 6-bromo-1-oxo-1,2,3,4-tetrahydroisoquinoline-3-carboxylate:

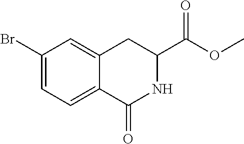 [SMILES: COC(=O)C1Cc2cc(Br)ccc2C(=O)N1]

To a stirred solution of methyl 3-(3-bromophenyl)-2-(((trichloromethoxy)carbonyl)amino)propanoate (14.8 g, 35.5 mmol, quant) in CH₂Cl₂ (150 mL) was added AlCl₃ (9.3 g, 71.1 mmol) portionwise at 0° C. The mixture was then heated at 45° C. for 3 h before cooling to RT, quenched with water (100 mL) and extracted with CH₂Cl₂ (2×300 mL). The combined organic layers were dried over Na₂SO₄ filtered and concentrated under reduced pressure to give crude residue. Purification by silica gel chromatography (100-200 mesh) using 0-60% EtOAc in hexane as an eluent afforded the product (2.9 g, 29%) as an off white solid. LCMS: [M+H]⁺=284.05.

Step 4: 6-bromo-3-(hydroxymethyl)-3,4-dihydroisoquinolin-1(2H)-one:

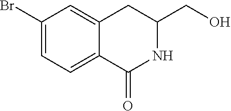 [SMILES: O=C1NC(CO)Cc2cc(Br)ccc21]

To a stirred solution of methyl 6-bromo-1-oxo-1,2,3,4-tetrahydroisoquinoline-3-carboxylate (2.9 g, 10.2 mmol) in EtOH (30 mL) was added portionwise LiBH₄ (450 mg, 20.4 mmol) at 0° C. The reaction was stirred at RT for 16 h. The solvent was concentrated under reduced pressure to give crude residue, which was then diluted with water (100 mL) and extracted with CH₂Cl₂ (2×300 mL). The combined organic layers were dried over Na₂SO₄ and concentrated under reduced pressure to give crude residue. The crude compound was triturated with diethyl ether (100 mL), filtered and dried under vacuum to afford the product (2.5 g, 95%) as an off-white solid. LCMS: [M+H]⁺=256.03.

Step 5: (6-bromo-1-oxo-1,2,3,4-tetrahydroisoquinolin-3-yl)methyl methanesulfonate:

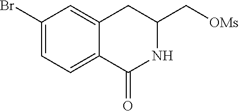 [SMILES: CS(=O)(=O)OCC1Cc2cc(Br)ccc2C(=O)N1]

To a stirred solution of 6-bromo-3-(hydroxymethyl)-3,4-dihydroisoquinolin-1(2H)-one (2.5 g, 9.8 mmol) in CH₂Cl₂ (30 mL) were added Et₃N (2.6 mL, 19.6 mmol), methanesulfonylchloride (1.5 mL, 19.6 mmol) and DMAP (110 mg, 0.98 mmol) at 0° C. Then, the reaction mixture was stirred at RT for 16 h. The solvent was concentrated under reduced pressure to give crude residue. The crude compound was diluted with water (100 mL) and extracted with CH₂Cl₂ (2×300 mL). The combined organic layers were dried over Na₂SO₄ and concentrated under reduced pressure to give crude material which was triturated with diethyl ether (100 mL) filtered and dried under vacuum to afford the product (2.2 g, 68%) as an off-white solid. LCMS: [M+H]⁺=334.03.

Step 6: 6-bromo-3-((methylamino)methyl)-3,4-dihydroisoquinolin-1(2H)-one:

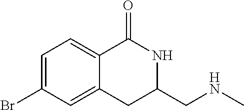

To a stirred solution of 6-bromo-1-oxo-1,2,3,4-tetrahydroisoquinolin-3-yl)methyl methanesulfonate (2.2 g, 6.6 mmol) in THF (25 mL) were added Et₃N (13.5 mL, 99 mmol), 40% aqueous methylamine (13.5 mL) at RT in a sealed tube. The reaction was stirred at 100° C. for 16 h before cooling to RT. The solvent was concentrated under reduced pressure to give crude residue. The crude compound was purified by column chromatography (neutral alumina) using 0-5% MeOH in DCM as an eluent to afford the product (0.85 g, 48%) as an off white solid. ¹H NMR (400 MHz, DMSO-d₆): δ 7.82 (s, 1H), 7.74 (d, J=8.4 Hz, 1H), δ 7.53 (dd, J=10.4, 2.4 Hz, 2H), δ 3.68-3.60 (m, 1H), δ 2.99 (dd, J=16.0, 4.8 Hz, 1H), δ 2.88-2.78 (m, 1H), 2.61-2.52 (m, 2H), δ 2.27 (s, 3H); LCMS: [M+H]⁺=269.04.

Step 7: 6-(6-amino-2-fluoro-5-(1-oxo-1,2,3,4-tetrahydroisoquinolin-6-yl)pyridin-3-yl)-3-((methylamino)methyl)-3,4-dihydroisoquinolin-1(2H)-one

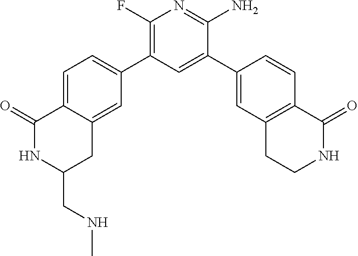 [SMILES: CNCC1Cc2cc(-c3cc(-c4ccc5c(c4)CCNC5=O)c(N)nc3F)ccc2C(=O)N1]

To a suspension of 6-(2-amino-6-fluoro-5-(4,4,5,5-tetramethyl-1,3,2-dioxaborolan-2-yl)pyridin-3-yl)-3,4-dihydroisoquinolin-1(2H)-one (85 mg, 0.223 mmol), 6-bromo-3-((methylamino)methyl)-3,4-dihydroisoquinolin-1(2H)-one (50 mg, 0.186 mmol), K₃PO₄ (118 mg, 0.557 mmol) and XPhos Pd G2 (17.54 mg, 0.022 mmol) was added a 5:1 mixture of butan-1-ol (4 ml): water (0.8 ml). The mixture was flushed with nitrogen and heated in the microwave at 90° C. for 2 h. The reaction was concentrated onto Celite® and purified by silica gel chromatography eluting with 0-10% MeOH/DCM+1% NH₄OH, followed by reverse phase chromatography (C18, 0-100% ACN/H₂O) to afford the title compound (30.6 mg, 37%) as beige solid. ¹H NMR (500 MHz, DMSO-d₆) δ 7.88 (br s, 1H), 7.84 (d, J=8.0 Hz, 1H), 7.78 (d, J=8.1 Hz, 1H), 7.65 (d, J=10.2 Hz, 1H), 7.62 (s, 1H), 7.47 (br d, J=8.1 Hz, 1H), 7.40-7.44 (m, 2H), 7.39 (s, 1H), 6.36 (s, 2H), 3.55-3.64 (m, 1H), 3.34 (td, J=6.4, 2.5 Hz, 3H), 2.94 (br dd, J=15.9, 4.5 Hz, 1H), 2.89 (br t, J=6.4 Hz, 2H), 2.77 (br dd, J=1 5.9, 9.3 Hz, 1H), 2.46-2.54 (m, 2H), 2.21 (s, 3H); LCMS: [M+H]⁺=446.67.

### Example 277: 6-(2-amino-6-fluoro-5-(4-(4-methyl-1,4-diazepan-1-yl)phenyl)pyridin-3-yl)-3,4-dihydroisoquinolin-1(2H)-one (I-277)

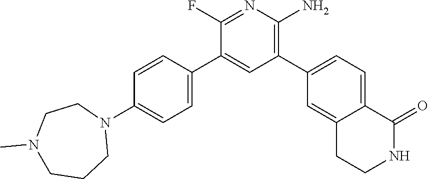 [SMILES: CN1CCCN(c2ccc(-c3cc(-c4ccc5c(c4)CCNC5=O)c(N)nc3F)cc2)CC1]

A procedure similar to Example 43, Step 5 using 6-(2-amino-5-bromo-6-fluoropyridin-3-yl)-3,4-dihydroisoquinolin-1(2H)-one (125 mg, 0.325 mmol), 1-(4-bromophenyl)-4-methyl-1,4-diazepane (73 mg, 0.271 mmol), [1,12-bis(diphenylphosphino)ferrocene]dichloropalladium(II) (19.84 mg, 0.027 mmol), aqueous K₃PO₄ (0.417 mL of a 1.3 M solution, 0.542 mmol), DME (4 mL) and H₂O (2 mL) afforded the title compound (52 mg, 43%) as a white solid. ¹H NMR (500 MHz, DMSO-d₆) δ 7.93 (br s, 1H), 7.90 (d, J=7.9 Hz, 1H), 7.56 (d, J=10.1 Hz, 1H), 7.48-7.43 (m, 2H), 7.34 (d, J=7.8 Hz, 2H), 6.73 (d, J=8.9 Hz, 2H), 6.10 (s, 2H), 3.55-3.50 (m, 2H), 3.45 (t, J=6.2 Hz, 2H), 3.40 (td, J=6.6, 2.6 Hz, 2H), 2.95 (t, J=6.5 Hz, 2H), 2.62-2.57 (m, 2H), 2.46-2.41 (m, 2H), 2.25 (s, 3H), 1.89 (quin, J=5.8 Hz, 2H); LCMS: [M+H]⁺=446.44.

### Example 278: 6-(2-amino-6-fluoro-5-(4-(4-isopropyl-3-methylpiperazin-1-yl)phenyl)pyridin-3-yl)-3,4-dihydroisoquinolin-1(2H)-one (I-278)

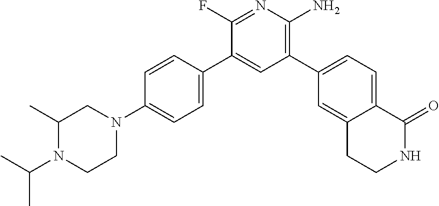 [SMILES: CC(C)N1CCN(c2ccc(-c3cc(-c4ccc5c(c4)CCNC5=O)c(N)nc3F)cc2)CC1C]

A procedure analogous to Example 43, Step 5 using 6-(2-amino-5-bromo-6-fluoropyridin-3-yl)-3,4-dihydroisoquinolin-1(2H)-one (133 mg, 0.347 mmol) and 4-(4-bromophenyl)-1-isopropyl-2-methylpiperazine (86 mg, 0.289 mmol) [1,12-bis(diphenylphosphino)ferrocene]dichloropalladium(II) (21.17 mg, 0.029 mmol), aqueous K₃PO₄ (0.445 mL of a 1.3 M solution, 0.579 mmol), DME (4 mL) and water (2 mL) afforded the title compound (67 mg, 48%) as a white solid. ¹H NMR (500 MHz, DMSO-d₆) δ 7.93 (br s, 1H), 7.90 (d, J=7.9 Hz, 1H), 7.57 (d, J=10.1 Hz, 1H), 7.49-7.42 (m, 2H), 7.38 (d, J=7.8 Hz, 2H), 6.95 (d, J=8.9 Hz, 2H), 6.16 (s, 2H), 3.58-3.47 (m, 2H), 3.40 (td, J=6.5, 2.6 Hz, 2H), 3.24-3.15 (m, 1H), 2.95 (t, J=6.5 Hz, 2H), 2.80 (dt, J=11.3, 3.0 Hz, 1H), 2.71 (td, J=11.0, 2.9 Hz, 1H), 2.65 (ddd, J=9.3, 6.2, 3.0 Hz, 1H), 2.45 (dd, J=11.2, 9.5 Hz, 1H), 2.35 (td, J=10.9, 2.8 Hz, 1H), 1.06 (dd, J=8.0, 6.5 Hz, 6H), 0.86 (d, J=6.5 Hz, 3H); LCMS: [M+H]⁺=474.30.

### Example 279: 6-(2-amino-6-fluoro-5-(4-(4-isopropyl-1,4-diazepan-1-yl)phenyl)pyridin-3-yl)-3,4-dihydroisoquinolin-1(2H)-one (I-279)

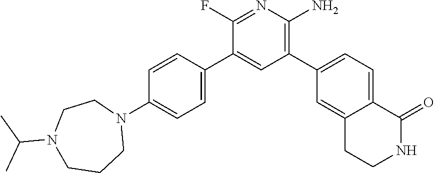 [SMILES: CC(C)N1CCCN(c2ccc(-c3cc(-c4ccc5c(c4)CCNC5=O)c(N)nc3F)cc2)CC1]

A procedure analogous to Example 43, Step 5 using 6-(2-amino-5-bromo-6-fluoropyridin-3-yl)-3,4-dihydroisoquinolin-1(2H)-one (139 mg, 0.363 mmol), 1-(4-bromophenyl)-4-isopropyl-1,4-diazepane (90 mg, 0.303 mmol), [1,12-bis(diphenylphosphino)ferrocene]dichloropalladium(II) (22.16 mg, 0.030 mmol), aqueous K₃PO₄ (0.466 mL of a 1.3 M solution, 0.606 mmol), DME (4 mL) and water (2 mL) afforded the title compound (30 mg, 21%) as a white solid. ¹H NMR (500 MHz, DMSO-d₆) δ 7.93 (br s, 1H), 7.90 (d, J=7.8 Hz, 1H), 7.56 (d, J=10.3 Hz, 1H), 7.46 (dd, J=7.9, 1.5 Hz, 1H), 7.44 (s, 1H), 7.33 (d, J=7.8 Hz, 2H), 6.72 (d, J=9.0 Hz, 2H), 6.09 (s, 2H), 3.49 (q, J=6.0 Hz, 4H), 3.40 (td, J=6.5, 2.7 Hz, 2H), 2.95 (t, J=6.5 Hz, 2H), 2.85 (dt, J=13.1, 6.5 Hz, 1H), 2.70-2.64 (m, 2H), 2.45-2.41 (m, 2H), 1.79 (quin, J=5.9 Hz, 2H), 0.92 (d, J=6.6 Hz, 6H); LCMS: [M+H]⁺=474.43.

### Example 280: 6-(2-amino-6-fluoro-5-(4-(hexahydropyrrolo[1,2-a]pyrazin-2(1H)-yl)phenyl)pyridin-3-yl)-3,4-dihydroisoquinolin-1(2H)-one (I-280)

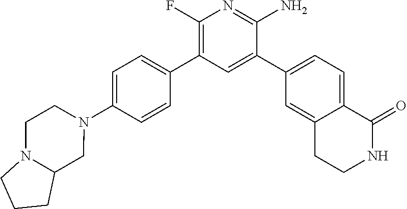 [SMILES: Nc1nc(F)c(-c2ccc(N3CCN4CCCC4C3)cc2)cc1-c1ccc2c(c1)CCNC2=O]

A procedure analogous to Example 43, Step 5 using 6-(2-amino-5-bromo-6-fluoropyridin-3-yl)-3,4-dihydroisoquinolin-1(2H)-one (116 mg, 0.303 mmol) and 2-(4-bromophenyl)octahydropyrrolo[1,2-a]pyrazine (71 mg, 0.252 mmol), [1,12-bis(diphenylphosphino)ferrocene]dichloropalladium(II) (18.48 mg, 0.025 mmol), aqueous K₃PO₄ (0.388 mL of a 1.3 M solution, 0.505 mmol), DME (4 mL) and water (2 mL) afforded the title compound (17 mg, 14%) as a white solid. ¹H NMR (500 MHz, DMSO-d₆) δ 7.93 (br s, 1H), 7.90 (d, J=7.9 Hz, 1H), 7.58 (d, J=10.3 Hz, 1H), 7.48-7.42 (m, 2H), 7.39 (d, J=7.8 Hz, 2H), 6.98 (d, J=8.9 Hz, 2H), 6.16 (s, 2H), 3.81 (br d, J=9.8 Hz, 1H), 3.66 (br d, J=11.9 Hz, 1H), 3.08-2.98 (m, 2H), 2.95 (t, J=6.5 Hz, 2H), 2.74 (td, J=11.7, 3.3 Hz, 1H), 2.40 (t, J=10.7 Hz, 1H), 2.22 (td, J=11.2, 3.1 Hz, 1H), 2.10-1.99 (m, 2H), 1.87-1.79 (m, 1H), 1.78-1.63 (m, 2H), 1.37 (qd, J=11.0, 6.8 Hz, 1H); LCMS: [M+H]⁺=458.63.

### Example 281: 6-(2-amino-6-fluoro-5-(4-(hexahydro-1H-pyrrolo[1,2-a][1,4]diazepin-2(3H)-yl)phenyl)pyridin-3-yl)-3,4-dihydroisoquinolin-1(2H)-one (I-281)

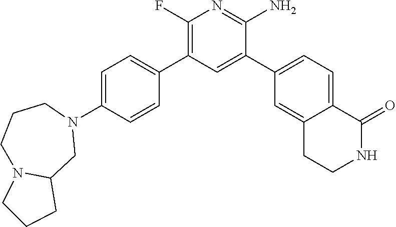 [SMILES: Nc1nc(F)c(-c2ccc(N3CCCN4CCCC4C3)cc2)cc1-c1ccc2c(c1)CCNC2=O]

A procedure similar to Example 43, Step 5 using 6-(2-amino-5-bromo-6-fluoropyridin-3-yl)-3,4-dihydroisoquinolin-1(2H)-one (125 mg, 0.325 mmol) and 2-(4-bromophenyl)octahydro-1H-pyrrolo[1,2-a][1,4]diazepine (80 mg, 0.271 mmol), [1,12-bis(diphenylphosphino)ferrocene]dichloropalladium(II) (19.83 mg, 0.027 mmol), aqueous K₃PO₄ (0.417 mL of a 1.3 M solution, 0.542 mmol), DME (4 mL) and water (2 mL) afforded the title compound (64 mg, 50%) as a white solid. ¹H NMR (500 MHz, DMSO-d₆) δ 7.93 (br s, 1H), 7.90 (d, J=7.9 Hz, 1H), 7.55 (d, J=10.1 Hz, 1H), 7.49-7.43 (m, 2H), 7.33 (d, J=7.8 Hz, 2H), 6.73 (d, J=9.0 Hz, 2H), 6.09 (s, 2H), 3.81 (dd, J=13.9, 2.2 Hz, 1H), 3.66 (dt, J =14.5, 5.5 Hz, 1H), 3.52-3.44 (m, 1H), 2.98-2.88 (m, 5H), 2.48-2.44 (m, 1H), 2.24 (q, J =9.0 Hz, 1H), 2.04 (td, J=12.0, 4.2 Hz, 1H), 1.98-1.87 (m, 2H), 1.81-1.70 (m, 1H), 1.70-1.59 (m, 2H), 1.46-1.34 (m, 1H); LCMS: [M+H]⁺=472.40.

### Example 282: (S)-6-(2-amino-5-(4-(2,4-dimethylpiperazin-1-yl)phenyl)-6-fluoropyridin-3-yl)-3,4-dihydroisoquinolin-1(2H)-one (I-282)

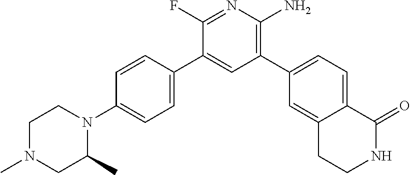 [SMILES: C[C@H]1CN(C)CCN1c1ccc(-c2cc(-c3ccc4c(c3)CCNC4=O)c(N)nc2F)cc1]

A procedure analogous to Example 43, Step 5 using 6-(2-amino-5-bromo-6-fluoropyridin-3-yl)-3,4-dihydroisoquinolin-1(2H)-one (137 mg, 0.357 mmol) and (S)-1-(4-bromophenyl)-2,4-dimethylpiperazine (80 mg, 0.297 mmol), [1,12-bis(diphenylphosphino)ferrocene]dichloropalladium(II) (21.75 mg, 0.030 mmol), aqueous K₃PO₄ (0.457 mL of a 1.3 M solution, 0.594 mmol), DME (4 mL) and water (2 mL) afforded the title compound (57 mg 43%) as a white solid. ¹H NMR (500 MHz, DMSO-d₆) δ 7.93 (br s, 1H), 7.90 (d, J=7.8 Hz, 1H), 7.58 (d, J=10.3 Hz, 1H), 7.49-7.42 (m, 2H), 7.39 (d, J=7.9 Hz, 2H), 6.91 (d, J=8.9 Hz, 2H), 6.15 (s, 2H), 4.05-3.97 (m, 1H), 3.42-3.39 (m, 2H), 3.31-3.26 (m, 1H), 3.02-2.91 (m, 3H), 2.79 (br dd, J=10.8, 1.3 Hz, 1H), 2.63 (br d, J=10.8 Hz, 1H), 2.24 (dd, J=10.9, 3.5 Hz, 1H), 2.19 (s, 3H), 2.04 (td, J=11.1, 3.4 Hz, 1H), 1.02 (d, J=6.5 Hz, 3H); LCMS: [M+H]⁺=446.44.

### Example 283: 6-(2-amino-5-(4-(4-(dimethylamino)piperidin-1-yl)phenyl)-6-fluoropyridin-3-yl)-3,4-dihydroisoquinolin-1(2H)-one (I-283)

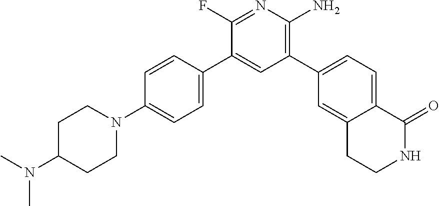 [SMILES: CN(C)C1CCN(c2ccc(-c3cc(-c4ccc5c(c4)CCNC5=O)c(N)nc3F)cc2)CC1]

A procedure similar to Example 43, Step 5 using 6-(2-amino-5-bromo-6-fluoropyridin-3-yl)-3,4-dihydroisoquinolin-1(2H)-one (117 mg, 0.305 mmol), 1-(4-bromophenyl)-N,N-dimethylpiperidin-4-amine (72 mg, 0.254 mmol), [1,12-bis(diphenylphosphino)ferrocene]dichloropalladium(II) (18.60 mg, 0.025 mmol), aqueous K₃PO₄ (0.391 mL of a 1.3 M solution, 0.508 mmol), DME (4 mL) and water (2 mL) afforded the title compound (46 mg, 39%) as a white solid. ¹H NMR (500 MHz, DMSO-d₆) δ 7.93 (br s, 1H), 7.90 (d, J=7.9 Hz, 1H), 7.58 (d, J=10.1 Hz, 1H), 7.49-7.42 (m, 2H), 7.38 (d, J=7.8 Hz, 2H), 6.97 (d, J=8.9 Hz, 2H), 6.16 (s, 2H), 3.74 (br d, J=12.6 Hz, 2H), 3.40 (td, J=6.5, 2.7 Hz, 2H), 2.95 (t, J=6.5 Hz, 2H), 2.72-2.64 (m, 2H), 2.23-2.20 (m, 1H), 2.18 (s, 6H), 1.82 (br d, J=12.0 Hz, 2H), 1.45 (qd, J=11.9, 3.9 Hz, 2H); LCMS: [M+H]⁺=460.51.

### Example 284: tert-butyl (2R,6S)-4-(4-(6-amino-2-fluoro-5-(1-oxo-1,2,3,4-tetrahydroisoquinolin-6-yl)pyridin-3-yl)phenyl)-2,6-dimethylpiperazine-1-carboxylate (I-284)

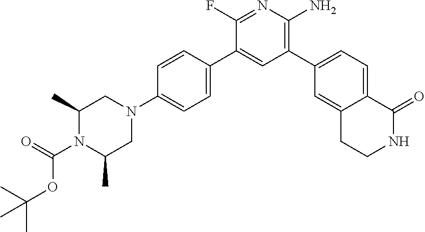 [SMILES: C[C@@H]1CN(c2ccc(-c3cc(-c4ccc5c(c4)CCNC5=O)c(N)nc3F)cc2)C[C@H](C)N1C(=O)OC(C)(C)C]

A procedure similar to Example 43, Step 5 using 6-(2-amino-5-bromo-6-fluoropyridin-3-yl)-3,4-dihydroisoquinolin-1(2H)-one (116 mg, 0.302 mmol), tert-butyl-4-(4-bromophenyl)-cis-2,6-dimethylpiperazine-1-carboxylate (93 mg, 0.252 mmol), [1,12-bis(diphenylphosphino)ferrocene]dichloropalladium(II) (18.43 mg, 0.025 mmol), aqueous K₃PO₄ (0.387 mL of a 1.3 M solution, 0.504 mmol), DME (4 mL) and water (2 mL) afforded the product (79 mg, 58%) as a white solid. ¹H NMR (500 MHz, DMSO-d₆) δ 7.93 (br s, 1H), 7.90 (d, J=7.8 Hz, 1H), 7.58 (d, J=10.1 Hz, 1H), 7.48-7.39 (m, 4H), 6.99 (d, J=8.9 Hz, 2H), 6.18 (s, 2H), 4.15-4.08 (m, 2H), 3.54 (br d, J=12.2 Hz, 2H), 3.40 (td, J=6.5, 2.7 Hz, 2H), 2.95 (t, J=6.5 Hz, 2H), 2.81 (dd, J=12.2, 4.2 Hz, 2H), 1.43 (s, 9H), 1.25 (d, J=6.8 Hz, 6H); LCMS: [M+H]⁺=546.58.

### Example 285: 6-(2-amino-6-fluoro-5-(4-morpholinophenyl)pyridin-3-yl)-3-((methylamino)methyl)-3,4-dihydroisoquinolin-1(2H)-one (I-285)

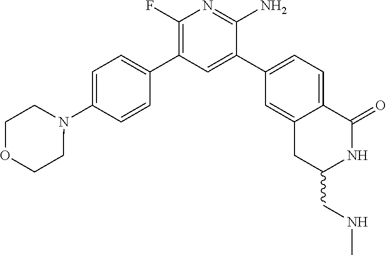 [SMILES: CNCC1Cc2cc(-c3cc(-c4ccc(N5CCOCC5)cc4)c(F)nc3N)ccc2C(=O)N1]

Step 1: tert-butyl ((6-bromo-1-oxo-1,2,3,4-tetrahydroisoquinolin-3-yl)methyl)(mehyl)carbamate

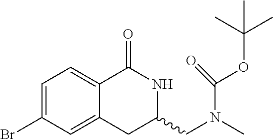 [SMILES: CN(CC1Cc2cc(Br)ccc2C(=O)N1)C(=O)OC(C)(C)C]

To a solution of 6-bromo-3-((methylamino)methyl)-3,4-dihydroisoquinolin-1(2H)-one (200 mg, 0.743 mmol) in CH₂Cl₂ (6 mL) at RT was added di-tert-butyl dicarbonate (178 mg, 0.817 mmol) solid in one portion, followed by N,N-diisopropylethylamine (0.272 ml, 1.561 mmol) and the reaction mixture was stirred at RT. After 1 h the reaction mixture was concentrated onto Celite®. Silica gel chromatography (eluting with 0-20% EtoAc/Hexanes) afforded the product as a colourless oil (359 mg, 82%). LCMS: [M+H]⁺=369.45.

Step 2: tert-butyl methyl((1-oxo-6-(4,4,5,5-tetramethyl-1,3,2-dioxaborolan-2-yl)-1,2,3,4-tetrahydroisoquinolin-3-yl)methyl)carbamate

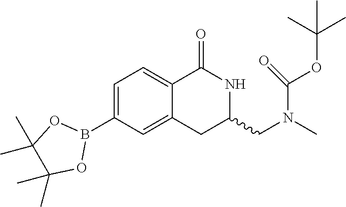 [SMILES: CN(CC1Cc2cc(B3OC(C)(C)C(C)(C)O3)ccc2C(=O)N1)C(=O)OC(C)(C)C]

A microwave vial containing a mixture of tert-butyl ((6-bromo-1-oxo-1,2,3,4-tetrahydroisoquinolin-3-yl)methyl)(methyl)carbamate (75 mg, 0.203 mmol), bis(pinacolato)diboron (58.3 mg, 0.230 mmol), [1,12-bis(diphenylphosphino)ferrocene]dichloropalladium(II) (14.86 mg, 0.020 mmol) and KOAc (59.8 mg, 0.609 mmol) in 1,4-dioxane (5 mL) was flushed with Ar. The vial was sealed and heated at 100° C. in a microwave reactor for 1 h. The resulting material was concentrated to obtain the crude product, which was taken to the next step without further purification. (84 mg, quantitative yield assuming100% conversion). LCMS: [M+H]⁺=417.65.

Step 3: tert-butyl ((6-(2-amino-5-bromo-6-fluoropyridin-3-yl)-1-oxo-1,2,3,4-tetrahydroisoquinolin-3-yl)methyl)(methyl)carbamate

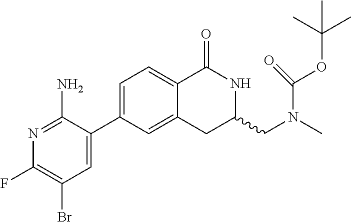 [SMILES: CN(CC1Cc2cc(-c3cc(Br)c(F)nc3N)ccc2C(=O)N1)C(=O)OC(C)(C)C]

A procedure analogous to that of Example 47, Step 3 was used, coupling 5-bromo-6-fluoro-3-iodopyridin-2-amine (75 mg, 0.237 mmol) with tert-butyl methyl((1-oxo-6-(4,4,5,5-tetramethyl-1,3,2-dioxaborolan-2-yl)-1,2,3,4-tetrahydroisoquinolin-3-yl)methyl)carbamate (84 mg, 0.202 mmol) with Na₂CO₃ monohydrate (61.8 mg, 0.498 mmol) and bis(triphenylphosphine)palladium(II) dichloride (19.99 mg, 0.028 mmol), in CH₃CN (3.5 mL) and water (1.3 mL) under microwave reaction conditions at 80° C. for 1 h. The reaction mixture was concentrated onto Celite®. Silica gel chromatography (eluting with 0-60% EtOAc/hexanes) afforded the product as a beige solid (89 mg, 78%). LCMS: [M+H]⁺=479.57.

Step 4: tert-butyl ((6-(2-amino-6-fluoro-5-(4-morpholinophenyl)pyridin-3-yl)-1-oxo-1,2,3,4-tetrahydroisoquinolin-3-yl)methyl)(methyl)carbamate

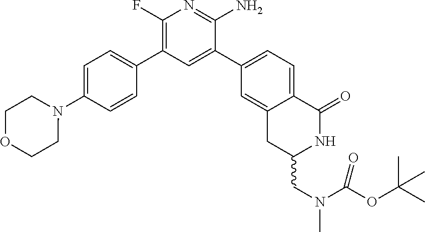 [SMILES: CN(CC1Cc2cc(-c3cc(-c4ccc(N5CCOCC5)cc4)c(F)nc3N)ccc2C(=O)N1)C(=O)OC(C)(C)C]

A procedure similar to Example 43, Step 5 using tert-butyl ((6-(2-amino-5-bromo-6-fluoropyridin-3-yl)-1-oxo-1,2,3,4-tetrahydroisoquinolin-3-yl)methyl)(methyl)carbamate (42 mg, 0.088 mmol) with 4-(morpholino)phenylboronic acid (25.4 mg, 0.123 mmol), [1,12-bis(diphenylphosphino)ferrocene]dichloropalladium(II) (9.62 mg, 0.013 mmol) and Cs₂CO₃ (86 mg, 0.263 mmol) in DME (4 mL) and water (1 mL). The reaction mixture was concentrated onto Celite®. Silica gel chromatography (eluting with 0-80% EtOAc/Hex) afforded the product (38 mg, 73%) as a pale yellow solid LCMS: [M+H]⁺=562.68.

Step 5: 6-(2-amino-6-fluoro-5-(4-morpholinophenyl)pyridin-3-yl)-3-((methylamino)methyl)-3,4-dihydroisoquinolin-1(2H)-one

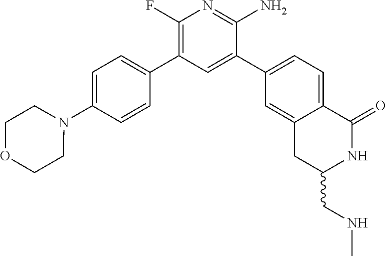 [SMILES: CNCC1Cc2cc(-c3cc(-c4ccc(N5CCOCC5)cc4)c(F)nc3N)ccc2C(=O)N1]

A solution of tert-butyl ((6-(2-amino-6-fluoro-5-(4-morpholinophenyl)pyridin-3-yl)-1-oxo-1,2,3,4-tetrahydroisoquinolin-3-yl)methyl)(methyl)carbamate (37 mg, 0.066 mmol) with TFA (0.050 mL, 0.659 mmol) was stirred in CH₂Cl₂ (1.5 mL) at RT for 1 h. The reaction mixture was concentrated and the methanol solution of the residue was passed through a Waters PoraPak CX cartridge to afford the title compound as a beige powder (27.5 mg, 25% based on purity of 86%). ¹H NMR (500 MHz, DMSO-d₆) δ 7.9-7.9 (m, 1H), 7.7-7.8 (m, 1H), 7.6-7.6 (m, 1H), 7.4-7.5 (m, 1H), 7.4-7.4 (m, 3H), 6.9-7.0 (m, 2H), 6.1-6.3 (m, 2H), 3.7-3.8 (m, 4H), 3.7-3.7 (m, 1H), 3.1-3.2 (m, 4H), 3.0-3.1 (m, 1H), 2.8-2.9 (m, 1H), 2.6-2.7 (m, 2H), 2.3-2.4 (m, 3H); LCMS: [M+H]⁺=462.71.

### Example 286: 6-(2-amino-6-fluoro-5-(4-(4-isopropylpiperazin-1-yl)phenyl)pyridin-3-yl)-3-((methylamino)methyl)-3,4-dihydroisoquinolin-1(2H)-one (I-286)

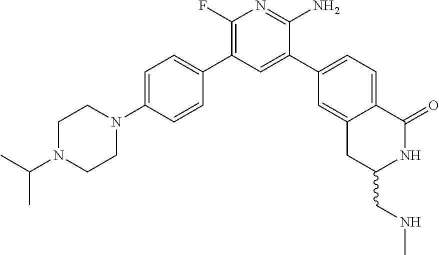 [SMILES: CNCC1Cc2cc(-c3cc(-c4ccc(N5CCN(C(C)C)CC5)cc4)c(F)nc3N)ccc2C(=O)N1]

Step 1: tert-butyl ((6-(2-amino-6-fluoro-5-(4-(4-isopropylpiperazin-1-yl)phenyl)pyridin-3-yl)-1-oxo-1,2,3,4-tetrahydroisoquinolin-3-yl)methyl)(methyl)carbamate

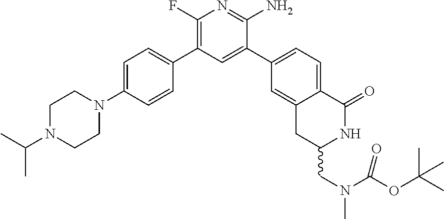 [SMILES: CC(C)N1CCN(c2ccc(-c3cc(-c4ccc5c(c4)CC(CN(C)C(=O)OC(C)(C)C)NC5=O)c(N)nc3F)cc2)CC1]

A procedure analogous to Example 43, Step 5 using tert-butyl ((6-(2-amino-5-bromo-6-fluoropyridin-3-yl)-1-oxo-1,2,3,4-tetrahydroisoquinolin-3 yl)methyl)(methyl)carbamate (41 mg, 0.086 mmol) 4-(4-isopropylpiperazinyl)phenylboronic acid, pinacol ester (39.5 mg, 0.120 mmol) gave the product as a brown solid (42 mg, 73% based on purity of 90%) LCMS: [M+H]⁺=603.85.

Step 2: 6-(2-amino-6-fluoro-5-(4-(4-isopropylpiperazin-1-yl)phenyl)pyridin-3-yl)-3-((methylamino)methyl)-3,4-dihydroisoquinolin-1(2H)-one

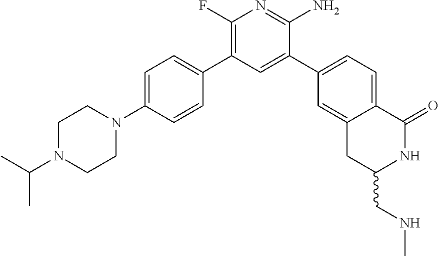 [SMILES: CNCC1Cc2cc(-c3cc(-c4ccc(N5CCN(C(C)C)CC5)cc4)c(F)nc3N)ccc2C(=O)N1]

A solution of tert-butyl ((6-(2-amino-6-fluoro-5-(4-(4-isopropylpiperazin-1-yl)phenyl)pyridin-3-yl)-1-oxo-1,2,3,4-tetrahydroisoquinolin-3-yl)methyl)(methyl)carbamate (42 mg, 0.070 mmol) in CH₂Cl₂ (1.5 mL) at RT was stirred with TFA (0.053 mL, 0.697 mmol) for 1 h. The reaction mixture was concentrated onto Celite®. Silica gel chromatography (eluting with DCM containing 0-5% MeOH and 0-0.5% NH₄OH), and the resulting crude product was passed through a Waters PoraPak CX cartridge to afford the title compound as a beige solid (23 mg, 62%). ¹H NMR (500 MHz, DMSO-d₆) δ 7.9-7.9 (m, 1H), 7.7-7.7 (m, 1H), 7.6-7.6 (m, 1H), 7.4-7.5 (m, 1H), 7.4-7.4 (m, 3H), 6.9-7.0 (m, 2H), 6.1-6.2 (m, 2H), 3.7-3.7 (m, 1H), 3.1-3.2 (m, 4H), 3.0-3.1 (m, 1H), 2.8-2.9 (m, 1H), 2.7-2.7 (m, 1H), 2.6-2.6 (m, 6H), 2.3-2.3 (m, 3H), 1.0-1.0 (m, 6H); LCMS: [M+H]⁺=503.64.

### Example 287: 6-(2-amino-6-fluoro-5-(4-(piperidin-4-yl)phenyl)pyridin-3-yl)-7-fluoro-3,4-dihydroisoquinolin-1(2H)-one (I-287)

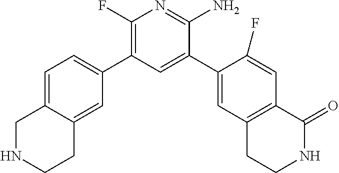 [SMILES: Nc1nc(F)c(-c2ccc3c(c2)CCNC3)cc1-c1cc2c(cc1F)C(=O)NCC2]

Step 1: tert-butyl 6-(6-amino-2-fluoro-5-(7-fluoro-1-oxo-1,2,3,4-tetrahydroisoquinolin-6-yl)pyridin-3-yl)-3,4-dihydroisoquinoline-2(1H)-carboxylate

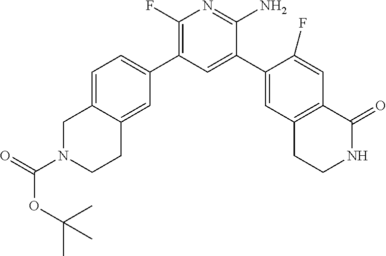 [SMILES: CC(C)(C)OC(=O)N1CCc2cc(-c3cc(-c4cc5c(cc4F)C(=O)NCC5)c(N)nc3F)ccc2C1]

To a suspension of 6-(2-amino-5-bromo-6-fluoropyridin-3-yl)-7-fluoro-3,4-dihydroisoquinolin-1(2H)-one (208 mg, 0.587 mmol), tert-butyl 6-(4,4,5,5-tetramethyl-1,3,2-dioxaborolan-2-yl)-3,4-dihydroisoquinoline-2(1 H)-carboxylate (232 mg, 0.646 mmol) and Cs₂CO₃ (478 mg, 1.47 mmol) in a mixture of DME (4 mL) and water (1.3 mL) was added PdCl₂dppf (43.0 mg, 0.059 mmol). The mixture was flushed with nitrogen heated in the microwave at 90° C. for 2.5 h. The reaction was concentrated onto Celite® and purified by silica gel chromatography, eluting with 0-10% MeOH/DCM+1% NH₄OH to afford the product (278 mg, 93%) as a beige solid. LCMS: [M+H]⁺=507.65.

Step 2: 6-(2-amino-6-fluoro-5-(4-(piperidin-4-yl)phenyl)pyridin-3-yl)-7-fluoro-3,4-dihydroisoquinolin-1(2H)-one

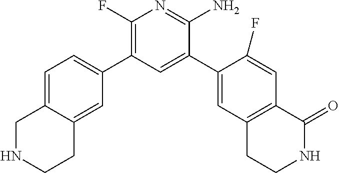 [SMILES: Nc1nc(F)c(-c2ccc3c(c2)CCNC3)cc1-c1cc2c(cc1F)C(=O)NCC2]

To a solution of tert-butyl 6-(6-amino-2-fluoro-5-(7-fluoro-1-oxo-1,2,3,4-tetrahydroisoquinolin-6-yl)pyridin-3-yl)-3,4-dihydroisoquinoline-2(1 H)-carboxylate (278 mg, 0.549 mmol) in CH₂Cl₂ (5.0 mL) was added TFA (1.26 mL, 16.46 mmol). The reaction was stirred at RT for 3 d. The mixture was concentrated onto Celite® and purified by reverse phase chromatography (C18, 0-100% MeCN—H₂O) to give the title compound (226 mg, 79%) as a white solid. ¹H NMR (500 MHz, DMSO-d₆) δ 8.81-8.96 (m, 1H), 8.13 (br s, 1H), 7.67 (d, J=10.2 Hz, 1H), 7.63 (d, J=10.0 Hz, 1H), 7.39-7.47 (m, 3H), 7.26 (d, J=8.0 Hz, 1H), 6.37 (s, 2H), 4.28 (s, 2H), 3.37-3.46 (m, 4H), 3.02 (s, 2H), 2.90-2.97 (m, 2H); LCMS: [M+H]+=407.55.

### Example 288: 6-(2-amino-6-fluoro-5-(4-((1-isopropylazetidin-3-yl)oxy)phenyl)pyridin-3-yl)-3,4-dihydroisoquinolin-1(2H)-one (I-288)

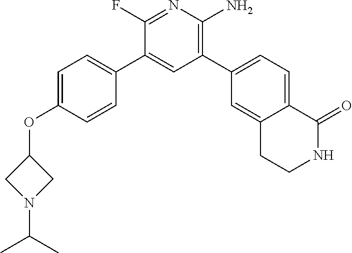 [SMILES: CC(C)N1CC(Oc2ccc(-c3cc(-c4ccc5c(c4)CCNC5=O)c(N)nc3F)cc2)C1]

A procedure similar to Example 218 using 6-(2-amino-5-(4-(azetidin-3-yloxy)phenyl)-6-fluoropyridin-3-yl)-3,4-dihydroisoquinolin-1(2H)-one (16.5 mg, 0.041 mmol) in CH₂Cl₂ (1.5 mL) and acetone (1.5 mL), 2 drops of glacial AcOH (14.70 mg, 0.245 mmol) and NaBH(OAc)₃ (51.9 mg, 0.245 mmol) afforded the title compound (6.8 mg, 36%) as a white powder. ¹H NMR (500 MHz, DMSO-d₆) δ 7.93 (brs, 1H), 7.90 (d, J=7.8 Hz, 1H), 7.59 (d, J=10.1 Hz, 1H), 7.50-7.42 (m, 4H), 6.88 (d, J=8.7 Hz, 2H), 6.23 (s, 2H), 4.79-4.70 (m, 1H), 3.74-3.67 (m, 2H), 3.44-3.38 (m, 2H), 2.99-2.90 (m, 4H), 2.35-2.28 (m, 1H), 0.88 (s, 3H), 0.87 (s, 3H); LCMS: [M+H]⁺=447.64.

### Example 289: 5-(6-amino-2-fluoro-5-(7-fluoro-1-oxo-1,2,3,4-tetrahydroisoquinolin-6-yl)pyridin-3-yl)-2-(piperidin-4-yloxy)benzonitrile (I-289)

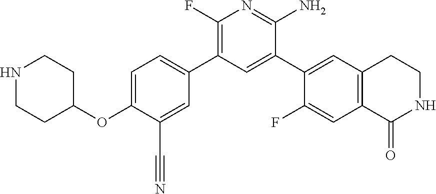 [SMILES: N#Cc1cc(-c2cc(-c3cc4c(cc3F)C(=O)NCC4)c(N)nc2F)ccc1OC1CCNCC1]

A procedure similar to Example 177, Step 1 using tert-butyl 4-(2-cyano-4-(4,4,5,5-tetramethyl-1,3,2-dioxaborolan-2-yl)phenoxy)piperidine-1-carboxylate (420 mg, 0.981 mmol), 6-(2-amino-5-bromo-6-fluoropyridin-3-yl)-7-fluoro-3,4-dihydroisoquinolin-1(2H)-one (289 mg, 0.817 mmol), 1,4-dioxane (3 mL) Cs₂CO₃ (799 mg, 2.451 mmol), water (1 mL) and Pd(dppf)Cl₂ (90 mg, 0.123 mmol) afforded the product (470 mg, 100%) as a beige powder which was used for the next step. LCMS: [M+H]⁺=576.60.

Step 2: 5-(6-amino-2-fluoro-5-(7-fluoro-1-oxo-1,2,3,4-tetrahydroisoquinolin-6-yl)pyridin-3-yl)-2-(piperidin-4-yloxy)benzonitrile

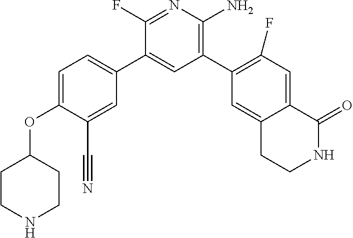 [SMILES: N#Cc1cc(-c2cc(-c3cc4c(cc3F)C(=O)NCC4)c(N)nc2F)ccc1OC1CCNCC1]

A procedure similar to Example 144, Step 3 using tert-butyl 4-(4-(6-amino-2-fluoro-5-(7-fluoro-1-oxo-1,2,3,4-tetrahydroisoquinolin-6-yl)pyridin-3-yl)-2-cyanophenoxy)piperidine-1-carboxylate (470 mg, 0.817 mmol), CH₂Cl₂ (4 mL) and TFA (2 mL, 26.1 mmol) afforded the title compound (298.2 mg, 73%) as a beige powder. ¹H NMR (500 MHz, DMSO-d₆) δ 8.11 (br s, 1H), 7.88 (d, J=2.2 Hz, 1H), 7.78 (br d, J=9.0 Hz, 1H), 7.72 (d, J=10.3 Hz, 1H), 7.61 (d, J=10.0 Hz, 1H), 7.43 (d, J=7.1 Hz, 1H), 7.35 (d, J=9.2 Hz, 1H), 6.37 (s, 2H), 4.73-4.64 (m, 1H), 3.43-3.38 (m, 2H), 2.93 (br d, J=6.6 Hz, 5H), 2.63-2.56 (m, 2H), 1.95-1.88 (m, 2H), 1.58-1.48 (m, 2H); LCMS: [M+H]⁺=476.54.

### Example 290: 6-(5-(4-((1-acetylpiperidin-4-yl)oxy)-3-fluorophenyl)-2-amino-6-fluoropyridin-3-yl)-3,4-dihydroisoquinolin-1(2H)-one (I-290)

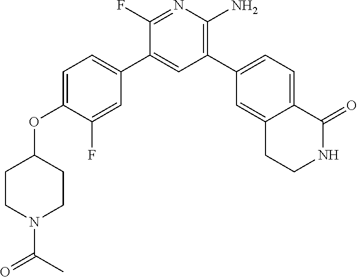 [SMILES: CC(=O)N1CCC(Oc2ccc(-c3cc(-c4ccc5c(c4)CCNC5=O)c(N)nc3F)cc2F)CC1]

A procedure similar to Example 191 using 6-(2-amino-6-fluoro-5-(3-fluoro-4-(piperidin-4-yloxy)phenyl)pyridin-3-yl)-3,4-dihydroisoquinolin-1(2H)-one (24 mg, 0.053 mmol) and N,N-diisopropylethylamine (0.046 mL, 0.266 mmol) in CH₂Cl₂ (3 ml) and acetic anhydride (5.04 μl, 0.053 mmol) afforded the title compound (21.7 mg, 79%) as a white powder. ¹H NMR (500 MHz, DMSO-d₆) δ 7.94 (br s, 1H), 7.90 (d, J=7.9 Hz, 1H), 7.66 (d, J=10.1 Hz, 1H), 7.50-7.43 (m, 3H), 7.36-7.25 (m, 2H), 6.33 (s, 2H), 4.70-4.60 (m, 1H), 3.87-3.78 (m, 1H), 3.71-3.62 (m, 1H), 3.40 (dt, J=6.5, 2.6 Hz, 2H), 3.38-3.34 (m, 1H), 3.25 (ddd, J=13.0, 8.9, 3.5 Hz, 1H), 3.00-2.91 (m, 2H), 2.02 (s, 3H), 2.00-1.94 (m, 1H), 1.93-1.86 (m, 1H), 1.71-1.61 (m, 1H), 1.59-1.49 (m, 1H); LCMS: [M+H]⁺=493.46.

### Example 291: 2-((1-acetylpiperidin-4-yl)oxy)-5-(6-amino-2-fluoro-5-(7-fluoro-1-oxo-1,2,3,4-tetrahydroisoquinolin-6-yl)pyridin-3-yl)benzonitrile (I-291)

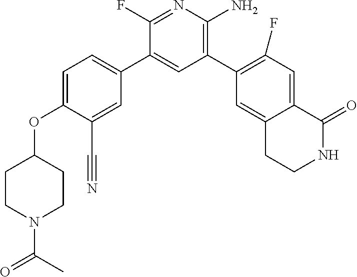 [SMILES: CC(=O)N1CCC(Oc2ccc(-c3cc(-c4cc5c(cc4F)C(=O)NCC5)c(N)nc3F)cc2C#N)CC1]

A procedure similar to Example 218 using 5-(6-amino-2-fluoro-5-(7-fluoro-1-oxo-1,2,3,4-tetrahydroisoquinolin-6-yl)pyridin-3-yl)-2-(piperidin-4-yloxy)benzonitrile (23.8 mg, 0.050 mmol) and N,N-diisopropylethylamine (0.044 mL, 0.250 mmol) in CH₂Cl₂ (3 mL) and acetic anhydride (4.73 μl, 0.050 mmol) afforded the title compound (20.6 mg, 76%) as a white powder. ¹H NMR (500 MHz, DMSO-d₆) δ 8.11 (br s, 1H), 7.91 (d, J=2.2 Hz, 1H), 7.82 (br d, J=8.9 Hz, 1H), 7.73 (d, J=10.3 Hz, 1H), 7.61 (d, J=10.0 Hz, 1H), 7.46-7.37 (m, 2H), 6.38 (s, 2H), 4.95-4.85 (m, 1H), 3.73-3.60 (m, 2H), 3.49-3.38 (m, 4H), 2.97-2.89 (m, 2H), 2.03 (s, 3H), 2.02-1.95 (m, 1H), 1.93-1.85 (m, 1H), 1.77-1.67 (m, 1H), 1.65-1.56 (m, 1H); LCMS: [M+H]⁺=518.52.

### Example 292: 5-(6-amino-2-fluoro-5-(7-fluoro-1-oxo-1,2,3,4-tetrahydroisoquinolin-6-yl)pyridin-3-yl)-2-((1-isopropylpiperidin-4-yl)oxy)benzonitrile (I-292)

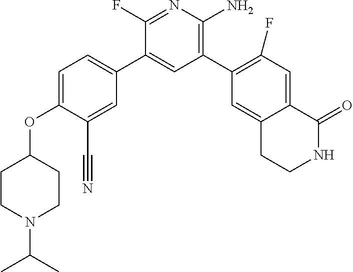 [SMILES: CC(C)N1CCC(Oc2ccc(-c3cc(-c4cc5c(cc4F)C(=O)NCC5)c(N)nc3F)cc2C#N)CC1]

A procedure similar to Example 218 using 5-(6-amino-2-fluoro-5-(7-fluoro-1-oxo-1,2,3,4-tetrahydroisoquinolin-6-yl)pyridin-3-yl)-2-(piperidin-4-yloxy)benzonitrile (24 mg, 0.050 mmol) in CH₂Cl₂ (1.5 mL) and acetone (1.5 ml), 2 drops of glacial AcOH (18.19 mg, 0.303 mmol) and NaBH(OAc)₃ (64.2 mg, 0.303 mmol) afforded the title compound (16.3 mg, 59%) as a white powder. ¹H NMR (500 MHz, DMSO-d₆) δ 8.11 (br s, 1H), 7.88 (d, J=2.2 Hz, 1H), 7.79 (br d, J=9.0 Hz, 1H), 7.72 (d, J=10.3 Hz, 1H), 7.60 (d, J=10.1 Hz, 1H), 7.43 (d, J=7.1 Hz, 1H), 7.34 (d, J=9.2 Hz, 1H), 6.37 (s, 2H), 4.69-4.61 (m, 1H), 3.44-3.37 (m, 2H), 2.96-2.89 (m, 2H), 2.74-2.66 (m, 3H), 2.41-2.33 (m, 2H), 1.98-1.89 (m, 2H), 1.74-1.62 (m, 2H), 0.98 (s, 3H), 0.97 (s, 3H); LCMS: [M+H]⁺=477.44.

### Example 293: 5-(6-amino-2-fluoro-5-(7-fluoro-1-oxo-1,2,3,4-tetrahydroisoquinolin-6-yl)pyridin-3-yl)-2-((1-(2,2,2-trifluoroethyl)piperidin-4-yl)oxy)benzonitrile (I-293)

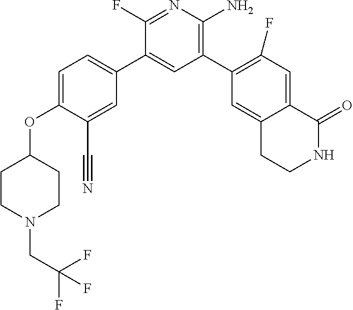 [SMILES: N#Cc1cc(-c2cc(-c3cc4c(cc3F)C(=O)NCC4)c(N)nc2F)ccc1OC1CCN(CC(F)(F)F)CC1]

A procedure similar to Example 186 using 5-(6-amino-2-fluoro-5-(7-fluoro-1-oxo-1,2,3,4-tetrahydroisoquinolin-6-yl)pyridin-3-yl)-2-(piperidin-4-yloxy)benzonitrile (23.8 mg, 0.050 mmol) and N,N-diisopropylethylamine (0.087 mL, 0.501 mmol) in DMF (1 mL) and 2,2,2-trifluoroethyl trifluoromethanesulfonate (23.23 mg, 0.100 mmol) afforded the title compound (20.4 mg, 69%) as a white powder. ¹H NMR (500 MHz, DMSO-d₆) δ 8.11 (br s, 1H), 7.89 (d, J=2.2 Hz, 1H), 7.79 (br d, J=9.0 Hz, 1H), 7.72 (d, J=10.3 Hz, 1H), 7.61 (d, J=10.1 Hz, 1H), 7.43 (d, J=7.0 Hz, 1H), 7.36 (d, J=9.0 Hz, 1H), 6.38 (s, 2H), 4.74-4.65 (m, 1H), 3.44-3.37 (m, 2H), 3.21 (q, J=10.2 Hz, 2H), 2.95-2.90 (m, 2H), 2.89-2.82 (m, 2H), 2.67-2.59 (m, 2H), 1.99-1.90 (m, 2H), 1.77-1.67 (m, 2H); LCMS: [M+H]⁺=558.54.

### Example 294: 6-(2-amino-6-fluoro-5-(4-(1-isopropyl-4-methoxypiperidin-4-yl)phenyl)pyridin-3-yl)-3,4-dihydroisoquinolin-1(2H)-one (I-294)

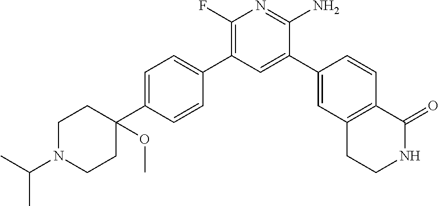 [SMILES: COC1(c2ccc(-c3cc(-c4ccc5c(c4)CCNC5=O)c(N)nc3F)cc2)CCN(C(C)C)CC1]

A solution of 4.0 M hydrogen chloride in dioxane (1.18 mL, 4.73 mmol) was added to a solution of tert-butyl 4-(4-(6-amino-2-fluoro-5-(1-oxo-1,2,3,4-tetrahydroisoquinolin-6-yl)pyridin-3-yl)phenyl)-4-methoxypiperidine-1-carboxylate (115 mg, 0.189 mmol), in MeOH (4 mL), and the reaction mixture was stirred at RT for 2.5 h. The reaction mixture was concentrated, dissolved in MeOH and passed through a Waters PoraPak CX column to afford 84 mg of the intermediate as a cream coloured solid. This intermediate was taken up in CH₂Cl₂ (5 mL) with propan-2-one (0.013 ml, 0.179 mmol) and glacial AcOH (2.56 μl, 0.045 mmol). NaBH(OAC)₃ (57.0 mg, 0.269 mmol) was added and the mixture was stirred overnight at RT and partitioned between saturated NaHCO₃ solution and CH₂Cl₂. The layers were separated and the aqueous layer was further extracted with CH₂Cl₂ (x 2). The combined extract was concentrated onto Celite®. Silica gel chromatography (eluting with CH₂Cl₂ containing 0-3% MeOH and 0-0.3% NH₄OH) afforded the title compound as a pale yellow powder (23 mg, 50%). ¹H NMR (500 MHz, DMSO-d₆) δ 8.00-7.92 (m, 1H), 7.92-7.86 (m, 1H), 7.72-7.62 (m, 1H), 7.59-7.52 (m, 2H), 7.50-7.47 (m, 1H), 7.47-7.45 (m, 1H), 7.44-7.38 (m, 2H), 6.32 (s, 2H), 3.45-3.40 (m, 2H), 3.02-2.93 (m, 2H), 2.93-2.84 (m, 3H), 2.73-2.65 (m, 1H), 2.63-2.56 (m, 2H), 2.49-2.43 (m, 2H), 2.00-1.91 (m, 2H), 1.90-1.75 (m, 2H), 1.07-0.93 (m, 6H); LCMS: [M+H]⁺=489.73

### Example 295: 6-(2-amino-6-fluoro-5-(4-(1-isopropyl-1,2,3,6-tetrahydropyridin-4-yl)phenyl)pyridin-3-yl)-7-fluoro-3,4-dihydroisoquinolin-1(2H)-one (I-295)

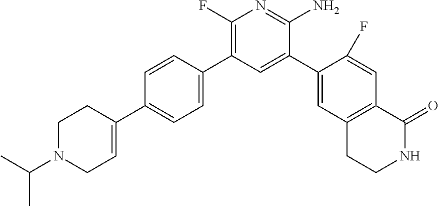 [SMILES: CC(C)N1CC=C(c2ccc(-c3cc(-c4cc5c(cc4F)C(=O)NCC5)c(N)nc3F)cc2)CC1]

Step 1: 1-isopropyl-4-(4-(4,4,5,5-tetramethyl-1,3,2-dioxaborolan-2-yl)phenyl)-1,2,3,6-tetrahydropyridine

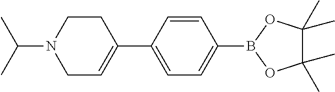 [SMILES: CC(C)N1CC=C(c2ccc(B3OC(C)(C)C(C)(C)O3)cc2)CC1]

A procedure analogous to Example 47, Step 2 using a mixture of 4-(4-bromophenyl)-1-isopropyl-1,2,3,6-tetrahydropyridine (150 mg, 0.535 mmol), bis(pinacolato)diboron (154 mg, 0.605 mmol), [1,12-bis(diphenylphosphino)ferrocene]dichloropalladium(II) (39.2 mg, 0.054 mmol) and KOAc (158 mg, 1.61 mmol) in 1,4-dioxane (6 mL) afforded the product (175 mg, 100%) which was dissolved in DME to make 10 mL stock solution with a concentrations of 17.5 mg/mL which was used in the next step. LCMS: [M+H]⁺=328.64 (stock solution).

Step 2: 6-(2-Amino-6-fluoro-5-(4-(1-isopropyl-1,2,3,6-tetrahydropyridin-4-yl)phenyl)pyridin-3-yl)-7-fluoro-3,4-dihydroisoquinolin-1(2H)-one

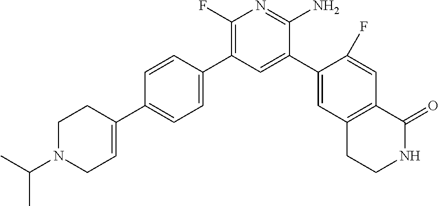 [SMILES: CC(C)N1CC=C(c2ccc(-c3cc(-c4cc5c(cc4F)C(=O)NCC5)c(N)nc3F)cc2)CC1]

A procedure similar to Example 43, Step 5 using 6-(2-amino-5-bromo-6-fluoropyridin-3-yl)-7-fluoro-3,4-dihydroisoquinolin-1(2H)-one (35 mg, 0.099 mmol), 1-isopropyl-4-(4-(4,4,5,5-tetramethyl-1,3,2-dioxaborolan-2-yl)phenyl)-1,2,3,6-tetrahydropyridine (2.40 mL of a stock solution prepared in Step 1, 0.128 mmol), [1,12-bis(diphenylphosphino)ferrocene]dichloropalladium(II) (10.85 mg, 0.015 mmol), Cs₂CO₃ (97 mg, 0.296 mmol) and DME (2.5 mL) afforded the title compound as a pale yellowish brown solid (16 mg, 32%)¹H NMR (500 MHz, DMSO-d₆) δ 8.16-8.07 (m, 1H), 7.69-7.64 (m, 1H), 7.63-7.59 (m, 1H), 7.55-7.39 (m, 5H), 6.40-6.26 (m, 2H), 6.25-6.16 (m, 1H), 3.44-3.40 (m, 2H), 3.22-3.14 (m, 2H), 2.97-2.89 (m, 2H), 2.85-2.74 (m, 1H), 2.72-2.61 (m, 2H), 2.49-2.43 (m, 2H), 1.13-0.96 (m, 6H); LCMS: [M+H]⁺=475.62

### Example 296: 6-(2-amino-5-(4-(1-ethyl-1,2,3,6-tetrahydropyridin-4-yl)phenyl)-6-fluoropyridin-3-yl)-7-fluoro-3,4-dihydroisoquinolin-1(2H)-one (I-296)

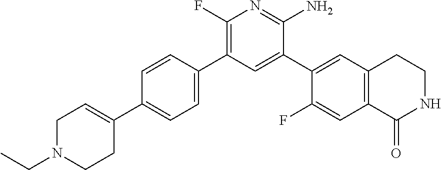 [SMILES: CCN1CC=C(c2ccc(-c3cc(-c4cc5c(cc4F)C(=O)NCC5)c(N)nc3F)cc2)CC1]

Step 2: tert-butyl 4-(4-(6-amino-2-fluoro-5-(7-fluoro-1-oxo-1,2,3,4-tetrahydroisoquinolin-6-yl)pyridin-3-yl)phenyl)-3,6-dihydropyridine-1(2H)-carboxylate

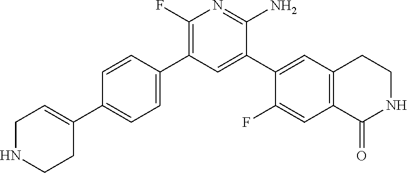 [SMILES: Nc1nc(F)c(-c2ccc(C3=CCNCC3)cc2)cc1-c1cc2c(cc1F)C(=O)NCC2]

A mixture of 6-(2-amino-5-bromo-6-fluoropyridin-3-yl)-7-fluoro-3,4-dihydroisoquinolin-1(2H)-one (200 mg, 0.565 mmol), (4-(1-(tert-Butoxycarbonyl)-1,2,3,6-tetrahydropyridin-4-yl)phenyl)boronic acid pinacol ester (261 mg, 0.678 mmol), [1,1′-Bis(diphenylphosphino)ferrocene]dichloropalladium(II) (62.0 mg, 0.085 mmol), and Cs₂CO₃ (552 mg, 1.694 mmol) in DME (8 ml) was flushed with argon. Water (3 ml) was added and the rxn mix was heated in a microwave reactor (high absorbance) at 90C for 1.75 h. The mixture was mixed with brine and EA, the org phase was separated, aq phase was extracted with EA, the combined org phase was dried over Na₂SO₄ and concentrated onto celite. Purification by sgc eluting with hexanes containing 0-100% EtOAc afforded the product as an off white solid (226 mg, 75% yield). LCMS: [M+H]⁺=533.72.

Step 2: 6-(2-amino-5-(4-(1-ethyl-1,2,3,6-tetrahydropyridin-4-yl)phenyl)-6-fluoropyridin-3-yl)-7-fluoro-3,4-dihydroisoquinolin-1(2H)-one

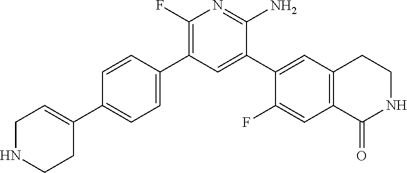 [SMILES: Nc1nc(F)c(-c2ccc(C3=CCNCC3)cc2)cc1-c1cc2c(cc1F)C(=O)NCC2]

tert-butyl 4-(4-(6-amino-2-fluoro-5-(7-fluoro-1-oxo-1,2,3,4-tetrahydroisoquinolin-6-yl)pyridin-3-yl)phenyl)-3,6-dihydropyridine-1(2H)-carboxylate (226 mg, 0.424 mmol) was stirred at RT with DCM (3 mL) and TFA (1 mL) for 1 h. Complete conversion to the desired product was observed. The mixture was concentrated to dryness, passed through a porapak cation exchange cartridge to isolate the desired product as an off white solid (176 mg). LCMS: [M+H]⁺=433.57.

Step 3: 6-(2-amino-5-(4-(1-ethyl-1,2,3,6-tetrahydropyridin-4-yl)phenyl)-6-fluoropyridin-3-yl)-7-fluoro-3,4-dihydroisoquinolin-1(2H)-one

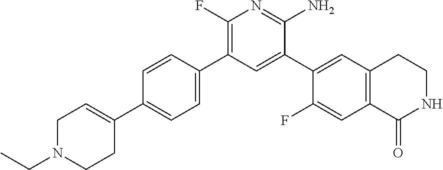 [SMILES: CCN1CC=C(c2ccc(-c3cc(-c4cc5c(cc4F)C(=O)NCC5)c(N)nc3F)cc2)CC1]

To a slurry of 6-(2-amino-6-fluoro-5-(4-(1,2,3,6-tetrahydropyridin-4-yl)phenyl)pyridin-3-yl)-7-fluoro-3,4-dihydroisoquinolin-1(2H)-one (45 mg, 0.104 mmol) in N,N-Dimethylformamide (DMF) (2 ml) at RT, was added 6-(2-amino-6-fluoro-5-(4-(1,2,3,6-tetrahydropyridin-4-yl)phenyl)pyridin-3-yl)-7-fluoro-3,4-dihydroisoquinolin-1 (2H)-one (45 mg, 0.104 mmol) and Bromoethane (0.023 ml, 0.312 mmol) and the rxn mixture was stirred o/n at RT. The volatiles (boromoethane and TEA) were removed on a rotavap. The DMF solution was diluted with 5 mL of water, the solid was filtered, the filter cake was washed with water (x2) and dried under high vac to isolate the title compound as a pale yellow solid (24 mg, 95% yield). ¹H NMR (500 MHz, DMSO-d₆) δ=8.16-8.06 (m, 1H), 7.70-7.64 (m, 1H), 7.64-7.59 (m, 1H), 7.57-7.38 (m, 5H), 6.38-6.26 (m, 2H), 6.25-6.17 (m, 1H), 3.50-3.39 (m, 4H), 3.16-3.02 (m, 2H), 2.95-2.88 (m, 2H), 2.68-2.59 (m, 2H), 2.47-2.40 (m, 2H), 1.13-0.97 (m, 3H); LCMS: [M+H]⁺=461.58.

### Example 297: 6-(2-amino-5-(4-(1-(cyclopropylmethyl)-1,2,3,6-tetrahydropyridin-4-yl)phenyl)-6-fluoropyridin-3-yl)-7-fluoro-3,4-dihydroisoquinolin-1(2H)-one (I-297)

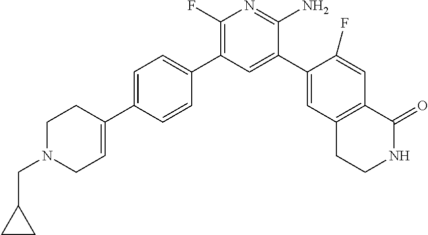 [SMILES: Nc1nc(F)c(-c2ccc(C3=CCN(CC4CC4)CC3)cc2)cc1-c1cc2c(cc1F)C(=O)NCC2]

A procedure similar to Example 218 using 6-(2-amino-6-fluoro-5-(4-(1,2,3,6-tetrahydropyridin-4-yl)phenyl)pyridin-3-yl)-7-fluoro-3,4-dihydroisoquinolin-1 (2H)-one (40 mg, 0.092 mmol) and (bromomethyl)cyclopropane (62.4 mg, 0.462 mmol) afforded the title compound (17 mg, 36%) as a pale yellow solid. ¹H NMR (500 MHz, DMSO-d₆) δ 7.99-7.93 (m, 1H), 7.56-7.50 (m, 1H), 7.49-7.45 (m, 1H), 7.40-7.30 (m, 4H), 7.29 (br d, J=7.0 Hz, 1H), 6.25-6.13 (m, 2H), 6.12-6.02 (m, 1H), 3.33-3.25 (m, 4H), 3.10-2.98 (m, 2H), 2.82-2.76 (m, 2H), 2.69-2.52 (m, 2H), 2.32-2.13 (m, 2H), 0.76 (br s, 1H), 0.46-0.28 (m, 2H), 0.09-0.15 (m, 2H); LCMS: [M+H]⁺=487.66.

### Example 298: 6-(2-amino-5-(4-chlorophenyl)-6-fluoropyridin-3-yl)-3-((methylamino)methyl)-3,4-dihydroisoquinolin-1(2H)-one (I-298)

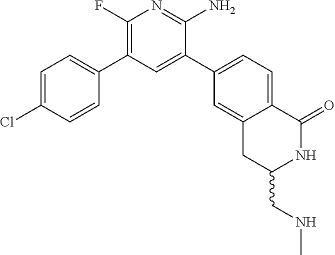 [SMILES: CNCC1Cc2cc(-c3cc(-c4ccc(Cl)cc4)c(F)nc3N)ccc2C(=O)N1]

Step 1: tert-butyl ((6-(2-amino-5-(4-chlorophenyl)-6-fluoropyridin-3-yl)-1-oxo-1,2,3,4-tetrahydroisoquinolin-3-yl)methyl)(methyl)carbamate

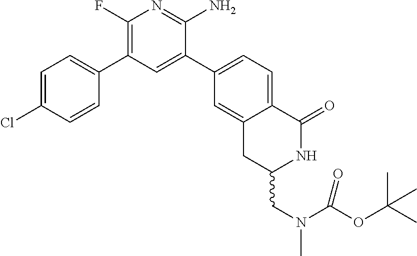 [SMILES: CN(CC1Cc2cc(-c3cc(-c4ccc(Cl)cc4)c(F)nc3N)ccc2C(=O)N1)C(=O)OC(C)(C)C]

A procedure analogous to Example 177, Step 1 using tert-butyl ((6-(2-amino-5-bromo-6-fluoropyridin-3-yl)-1-oxo-1,2,3,4-tetrahydroisoquinolin-3-yl)methyl)(methyl)carbamate (48 mg, 0.100 mmol), 4-chlorophenylboronic acid, pinacol ester (33.4 mg, 0.140 mmol), [1,12-bis(diphenylphosphino)ferrocene]dichloropalladium(II) (10.99 mg, 0.015 mmol) and Cs₂CO₃ (98 mg, 0.300 mmol) in DME (4 mL) and water (1 mL) afforded the title compound as an off white solid (42.5 mg, 83%). LCMS: [M+H]⁺=511.54.

Step 2: 6-(2-amino-5-(4-chlorophenyl)-6-fluoropyridin-3-yl)-3-((methylamino)methyl)-3,4-dihydroisoquinolin-1(2H)-one

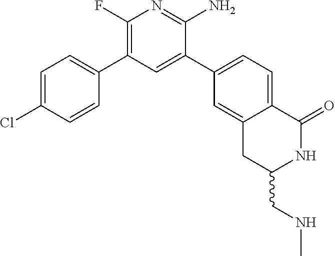 [SMILES: CNCC1Cc2cc(-c3cc(-c4ccc(Cl)cc4)c(F)nc3N)ccc2C(=O)N1]

A solution of tert-butyl ((6-(2-amino-5-(4-chlorophenyl)-6-fluoropyridin-3-yl)-1-oxo-1,2,3,4-tetrahydroisoquinolin-3-yl)methyl)(methyl)carbamate (42.5 mg, 0.083 mmol) in DCM (1.5 mL) and TFA (0.053 mL, 0.697 mmol) in CH₂Cl₂ (1.5 mL) was stirred at RT with for 1 h. The reaction mixture was concentrated to dryness and the methanol solution of the residue was passed through a Waters PoraPak CX cartridge to afford the title compound as an off-white solid (26 mg, 72%). ¹H NMR (500 MHz, DMSO-d₆) δ 7.94-7.88 (m, 1H), 7.81-7.74 (m, 1H), 7.71-7.65 (m, 1H), 7.64-7.55 (m, 2H), 7.53-7.40 (m, 4H), 6.44-6.34 (m, 2H), 3.72 (br s, 1H), 3.09-3.01 (m, 1H), 2.91-2.82 (m, 1H), 2.70-2.63 (m, 2H), 2.33 (s, 3H); LCMS: [M+H]⁺=411.44.

### Example 299: 6-(2-amino-6-fluoro-5-(2-isopropyl-1,2,3,4-tetrahydroisoquinolin-6-yl)pyridin-3-yl)-7-fluoro-3,4-dihydroisoquinolin-1(2H)-one (I-299)

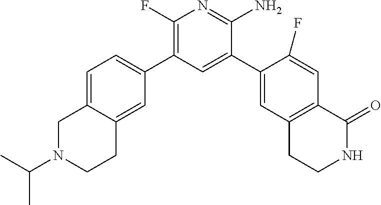 [SMILES: CC(C)N1CCc2cc(-c3cc(-c4cc5c(cc4F)C(=O)NCC5)c(N)nc3F)ccc2C1]

To a solution of 6-(2-amino-6-fluoro-5-(1,2,3,4-tetrahydroisoquinolin-6-yl)pyridin-3-yl)-7-fluoro-3,4-dihydroisoquinolin-1(2H)-one, trifluoroacetate (25 mg, 0.048 mmol) and N,N-diisopropylethylamine (0.084 ml, 0.480 mmol) in DMF (1.0 mL) was added 2-iodopropane (40.8 mg, 0.240 mmol). The reaction was stirred at RT for 16 h, concentrated onto Celite® and purified by silica gel chromatography eluting with 0-10% MeOH/DCM+1% NH₄OH, followed by reverse phase chromatography (C18, 0-100% ACN/H₂O) to afford the title compound (12.2 mg, 57%) as a yellow solid. ¹H NMR (500 MHz, DMSO-d₆) δ 8.11 (br s, 1H), 7.62 (dd, J=10.1, 2.63 Hz, 2H), 7.43 (d, J=7.1 Hz, 1H), 7.23-7.30 (m, 2H), 7.10 (d, J=8.0 Hz, 1H), 6.27 (s, 2H), 3.64 (s, 2H), 3.40-3.43 (m, 2H), 2.93 (br t, J=6.3 Hz, 2H), 2.83-2.89 (m, 1H), 2.81 (br t, J=5.5 Hz, 2H), 2.67-2.73 (m, 2H), 1.06 (d, J=6.5 Hz, 6H); LCMS: [M+H]⁺=449.61.

### Example 300: 6-(2-amino-6-fluoro-5-(2-(2-methoxyethyl)-1,2,3,4-tetrahydroisoquinolin-6-yl)pyridin-3-yl)-7-fluoro-3,4-dihydroisoquinolin-1(2H)-one (I-300)

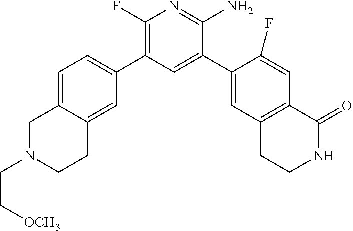 [SMILES: COCCN1CCc2cc(-c3cc(-c4cc5c(cc4F)C(=O)NCC5)c(N)nc3F)ccc2C1]

A procedure similar to Example 186 using 6-(2-amino-6-fluoro-5-(1,2,3,4-tetrahydroisoquinolin-6-yl)pyridin-3-yl)-7-fluoro-3,4-dihydroisoquinolin-1(2H)-one, trifluoroacetate (25 mg, 0.048 mmol) and N,N-diisopropylethylamine (0.084 ml, 0.480 mmol) in DMF (1.0 mL) and 2-bromoethyl methyl ether (33.4 mg, 0.240 mmol) afforded the title compound (14.5 mg, 65%) as a yellow solid. ¹H NMR (500 MHz, DMSO-d₆) δ 8.11 (br s, 1H), 7.62 (dd, J=10.1, 4.7 Hz, 2H), 7.43 (d, J=7.1 Hz, 1H), 7.23-7.32 (m, 2H), 7.08 (d, J=8.0 Hz, 1H), 6.28 (s, 2H), 3.61 (s, 2H), 3.53 (t, J=5.9 Hz, 2H), 3.41 (td, J=6.5, 2.6 Hz, 2H), 3.27 (s, 3H), 2.93 (br t, J=6.4 Hz, 2H), 2.80-2.85 (m, 2H), 2.69-2.74 (m, 2H), 2.65 (t, J=5.8 Hz, 2H); LCMS: [M+H]⁺=465.60.

### Example 301: 6-(2-amino-6-fluoro-5-(4-((1R,5S)-3-isopropyl-3-azabicyclo[3.1.0]hexan-1-yl)phenyl)pyridin-3-yl)-8-fluoro-3,4-dihydroisoquinolin-1(2H)-one (I-301)

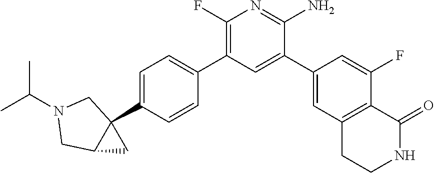 [SMILES: CC(C)N1C[C@H]2C[C@@]2(c2ccc(-c3cc(-c4cc(F)c5c(c4)CCNC5=O)c(N)nc3F)cc2)C1]

Step 1: (1R,5S)-3-isopropyl-1-(4-(4,4,5,5-tetramethyl-1,3,2-dioxaborolan-2-yl)phenyl)-3-azabicyclo[3.1.0]hexane

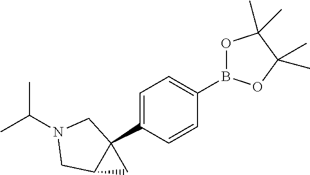 [SMILES: CC(C)N1C[C@H]2C[C@@]2(c2ccc(B3OC(C)(C)C(C)(C)O3)cc2)C1]

A mixture of (1R,5S)-1-(4-bromophenyl)-3-isopropyl-3-azabicyclo[3.1.0]hexane (235 mg, 0.839 mmol), bis(pinacolato)diboron (241 mg, 0.948 mmol, preparation described in Example 110, Step 1), [1,12-bis(diphenylphosphino)ferrocene]dichloropalladium(II) (61.4 mg, 0.084 mmol) and KOAc (247 mg, 2.52 mmol) in 1,4-dioxane (10 mL), was flushed with argon. The reaction mixture was heated at 100° C. in a microwave reactor for 1 h. The reaction mixture was filtered through a thin pad of Celite®. The filtrate was concentrated to dryness, and the residue was dissolved in DME to make a 10 mL stock solution with the concentration estimated to be 27.4 mg/mL. LCMS: [M+H]⁺=328.70.

Step 2: 8-fluoro-6-(4,4,5,5-tetramethyl-1,3,2-dioxaborolan-2-yl)-3,4-dihydroisoquinolin-1(2H)-one

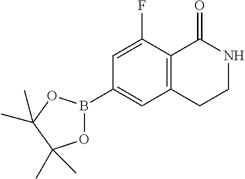 [SMILES: CC1(C)OB(c2cc(F)c3c(c2)CCNC3=O)OC1(C)C]

To a 100 mL round bottom flask charged with 6-bromo-8-fluoro-3,4-dihydroisoquinolin-1(2H)-one (896 mg, 3.67 mmol), bis(pinacolato)diboron (1025 mg, 4.04 mmol) and KOAc (1081 mg, 11.01 mmol), was added anhydrous 1,4-dioxane (40.0 ml). The system was flushed with nitrogen then PdCl₂dppf (269 mg, 0.367 mmol) was added. The mixture was further flushed with nitrogen then heated at 100° C. overnight. LCMS analysis showed a 58:42 mixture of the boronic acid and boronate. The reaction was diluted with acetonitrile, filtered through a pad of Celite, and concentrated in vacuo. The crude material was used without further purification in the next step. Boronate LCMS: [M+H]⁺=292.34, boronic acid LCMS: [M+H]⁺=210.39.

Step 3: 6-(2-amino-5-bromo-6-fluoropyridin-3-yl)-8-fluoro-3,4-dihydroisoquinolin-1(2H)-one

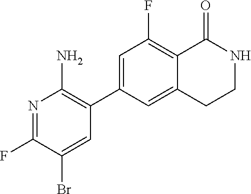 [SMILES: Nc1nc(F)c(Br)cc1-c1cc(F)c2c(c1)CCNC2=O]

To a solution of 5-bromo-6-fluoro-3-iodopyridin-2-amine (1280 mg, 4.04 mmol) and 8-fluoro-6-(4,4,5,5-tetramethyl-1,3,2-dioxaborolan-2-yl)-3,4-dihydroisoquinolin-1(2H)-one (1069 mg, 3.67 mmol) in CH₃CN (15 mL), in a microwave vial, was added a solution of Na₂CO₃—H₂O (956 mg, 7.71 mmol) in water (3.75 mL). The system was flushed with nitrogen and bis(triphenylphosphine)palladium(II) dichloride (309 mg, 0.441 mmol) was added. The reaction was further flushed with nitrogen then heated in the microwave at 80° C. for 3 h. The reaction was concentrated onto Celite® and purified by silica gel chromatography eluting with 0-100% EtOAc/Hexanes to give the product (872 mg, 67%) as a beige solid. LCMS: [M+H]⁺=354.40.

Step 4: 6-(2-amino-6-fluoro-5-(4-((1R,5S)-3-isopropyl-3-azabicyclo[3.1.0]hexan-1-yl)phenyl)pyridin-3-yl)-8-fluoro-3,4-dihydroisoquinolin-1(2H)-one

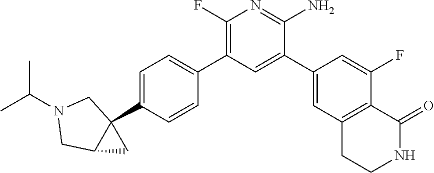 [SMILES: CC(C)N1C[C@H]2C[C@@]2(c2ccc(-c3cc(-c4cc(F)c5c(c4)CCNC5=O)c(N)nc3F)cc2)C1]

A procedure similar to Example 177, Step 1 using 6-(2-amino-5-bromo-6-fluoropyridin-3-yl)-8-fluoro-3,4-dihydroisoquinolin-1(2H)-one (40 mg, 0.113 mmol), (1R,5S)-3-isopropyl-1-(4-(4,4,5,5-tetramethyl-1,3,2-dioxaborolan-2-yl)phenyl)-3-azabicyclo[3.1.0]hexane (1.75 mL, 0.147 mmol, 27.5 mg/ml in ACN) and Cs₂CO₃ (92 mg, 0.282 mmol) in DME (2 mL) and water (0.67 mL), and PdCl₂dppf (8.26 mg, 0.011 mmol) afforded the title compound (26.9 mg, 50%) as a beige solid. ¹H NMR (500 MHz, DMSO-d₆) b 8.00 (br s, 1H), 7.66 (d, J=10.2 Hz, 1H), 7.47 (d, J=7.2 Hz, 2H), 7.30 (s, 1H), 7.26 (br d, J=12.0 Hz, 1H), 7.20 (d, J=8.4 Hz, 2H), 6.40 (s, 2H), 3.05 (d, J=8.6 Hz, 1H), 2.94 (br t, J=6.3 Hz, 2H), 2.57 (d, J=8.4 Hz, 1H), 2.43-2.49 (m, 2H), 1.81 (dt, J=7.8, 3.8 Hz, 1H), 1.31 (t, J=3.9 Hz, 1H), 1.04 (d, J=6.4 Hz, 3H), 1.01 (d, J=6.2 Hz, 3H), 0.74 (dd, J=7.9, 3.6 Hz, 1H); LCMS: [M+H]⁺=475.62.

### Example 302: 6-(2-amino-6-fluoro-5-(4-(1-isopropylpiperidin-4-yl)phenyl)pyridin-3-yl)-8-fluoro-3,4-dihydroisoquinolin-1(2H)-one (I-302)

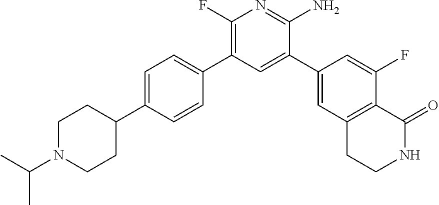 [SMILES: CC(C)N1CCC(c2ccc(-c3cc(-c4cc(F)c5c(c4)CCNC5=O)c(N)nc3F)cc2)CC1]

Step 1: tert-butyl 4-(4-(6-amino-2-fluoro-5-(8-fluoro-1-oxo-1,2,3,4-tetrahydroisoquinolin-6-yl)pyridin-3-yl)phenyl)piperidine-1-carboxylate

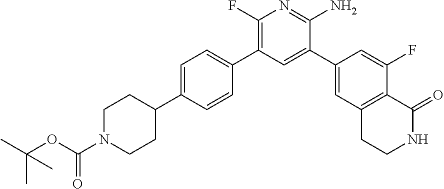 [SMILES: CC(C)(C)OC(=O)N1CCC(c2ccc(-c3cc(-c4cc(F)c5c(c4)CCNC5=O)c(N)nc3F)cc2)CC1]

To a suspension of 6-(2-amino-5-bromo-6-fluoropyridin-3-yl)-8-fluoro-3,4-dihydroisoquinolin-1(2H)-one (130 mg, 0.367 mmol), 4-(N-Boc-piperidino)phenylboronic acid pinacol ester (171 mg, 0.440 mmol) and Cs₂CO₃ (299 mg, 0.918 mmol) in DME (5 mL) and water (1.67 mL) was added PdCl₂dppf (26.9 mg, 0.037 mmol). The mixture was flushed with nitrogen and heated in the microwave at 90° C. for 2.5 h. The reaction was concentrated onto Celite® and purified by silica gel chromatography (eluting with 0-10% MeOH/DCM+1% NH₄OH) to afford the product (196 mg, 100%) as a brown solid. LCMS: [M+H]⁺=535.67.

Step 2: 6-(2-amino-6-fluoro-5-(4-(piperidin-4-yl)phenyl)pyridin-3-yl)-8-fluoro-3,4-dihydroisoquinolin-1(2H)-one

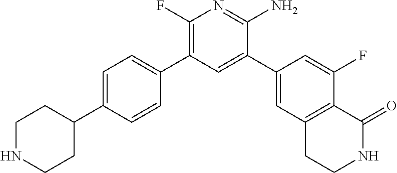 [SMILES: Nc1nc(F)c(-c2ccc(C3CCNCC3)cc2)cc1-c1cc(F)c2c(c1)CCNC2=O]

To a solution of tert-butyl 4-(4-(6-amino-2-fluoro-5-(8-fluoro-1-oxo-1,2,3,4-tetrahydroisoquinolin-6-yl)pyridin-3-yl)phenyl)piperidine-1-carboxylate (196 mg, 0.367 mmol) in CH₂Cl₂ (5.0 ml) was added TFA (0.84 mL, 11.0 mmol). The reaction was stirred at RT for 72 h then concentrated onto Celite® and purified by reverse phase chromatography (C18, 0-100% MeCN—H₂O) to give the product as the TFA salt (154 mg, 77%) as beige solid. LCMS: [M+H]⁺=435.53.

Step 3: 6-(2-amino-6-fluoro-5-(4-(1-isopropylpiperidin-4-yl)phenyl)pyridin-3-yl)-8-fluoro-3,4-dihydroisoquinolin-1(2H)-one

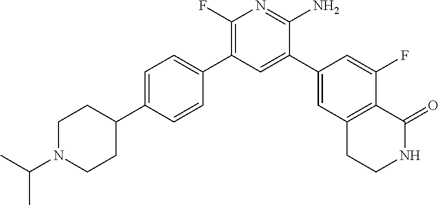 [SMILES: CC(C)N1CCC(c2ccc(-c3cc(-c4cc(F)c5c(c4)CCNC5=O)c(N)nc3F)cc2)CC1]

To a solution of 6-(2-amino-6-fluoro-5-(4-(piperidin-4-yl)phenyl)pyridin-3-yl)-8-fluoro-3,4-dihydroisoquinolin-1(2H)-one, trifluoroacetate (29 mg, 0.053 mmol) and N,N-diisopropylethylamine (0.092 ml, 0.529 mmol) in DMF (1.0 mL) was added 2-iodopropane (44.9 mg, 0.264 mmol). The reaction was stirred at RT for 96 h, then concentrated onto Celite® and purified by silica gel chromatography eluting with 0-10% MeOH/DCM+1% NH₄OH to give the title compound (11.9 mg, 47%) as a white solid. ¹H NMR (500 MHz, DMSO-d₆) δ 7.99 (br s, 1H), 7.67 (d, J=10.2 Hz, 1H), 7.48 (d, J=7.3 Hz, 2H), 7.24-7.33 (m, 4H), 6.39 (s, 2H), 2.94 (br t, J=6.3 Hz, 2H), 2.89 (br d, J=11.4 Hz, 2H), 2.71 (dt, J=13.1, 6.6 Hz, 1H), 2.44-2.49 (m, 1H), 2.18-2.26 (m, 2H), 1.77 (br d, J=12.8 Hz, 2H), 1.62 (qd, J=12.3, 3.5 Hz, 2H), 1.24 (s, 1H), 1.00 (d, J=6.60 Hz, 6H); LCMS: [M+H]⁺=477.63.

### Example 303: 5-(6-amino-2-fluoro-5-(7-fluoro-1-oxo-1,2,3,4-tetrahydroisoquinolin-6-yl)pyridin-3-yl)-2-((1-(2-methoxyethyl)piperidin-4-yl)oxy)benzonitrile (I-303)

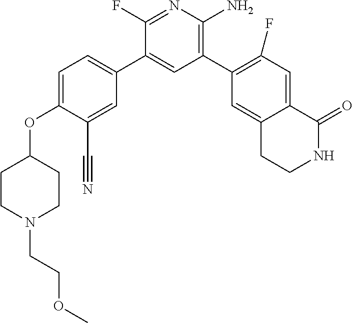 [SMILES: COCCN1CCC(Oc2ccc(-c3cc(-c4cc5c(cc4F)C(=O)NCC5)c(N)nc3F)cc2C#N)CC1]

A procedure similar to Example 186 using 5-(6-amino-2-fluoro-5-(7-fluoro-1-oxo-1,2,3,4-tetrahydroisoquinolin-6-yl)pyridin-3-yl)-2-(piperidin-4-yloxy)benzonitrile (26 mg, 0.055 mmol) and N,N-diisopropylethylamine (0.095 mL, 0.547 mmol) in DMF (1 mL) and 2-bromoethyl methyl ether (0.015 mL, 0.164 mmol) afforded the title compound (21.4 mg, 70%) as a beige powder. ¹H NMR (500 MHz, DMSO-d₆) δ 8.11 (br s, 1H), 7.89 (d, J=2.0 Hz, 1H), 7.79 (br d, J=8.9 Hz, 1H), 7.72 (d, J=10.3 Hz, 1H), 7.61 (d, J=10.0 Hz, 1H), 7.43 (d, J=7.1 Hz, 1H), 7.35 (d, J=9.0 Hz, 1H), 6.38 (s, 2H), 4.73-4.62 (m, 1H), 3.52-3.43 (m, 2H), 3.41 (dt, J=6.6, 2.9 Hz, 2H), 3.24 (s, 3H), 2.92 (br t, J=6.5 Hz, 2H), 2.83-2.61 (m, 2H), 2.60-2.51 (m, 2H), 2.44-2.25 (m, 2H), 2.03-1.89 (m, 2H), 1.79-1.64 (m, 2H); LCMS: [M +H]⁺=534.62.

### Example 304: 6-(5-(4-((1-acetylpiperidin-4-yl)oxy)-2-methoxyphenyl)-2-amino-6-fluoropyridin-3-yl)-3,4-dihydroisoquinolin-1(2H)-one (I-304)

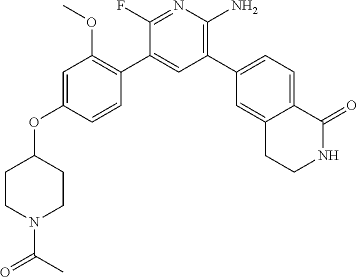 [SMILES: COc1cc(OC2CCN(C(C)=O)CC2)ccc1-c1cc(-c2ccc3c(c2)CCNC3=O)c(N)nc1F]

A procedure similar to Example 191 using 6-(2-amino-6-fluoro-5-(2-methoxy-4-(piperidin-4-yloxy)phenyl)pyridin-3-yl)-3,4-dihydroisoquinolin-1(2H)-one (25 mg, 0.054 mmol) and N,N-diisopropylethylamine (0.047 ml, 0.270 mmol) in CH₂Cl₂ (3 mL) and acetic anhydride (5.11 μl, 0.054 mmol) afforded the title compound (19.6 mg, 68%) as a white powder. ¹H NMR (500 MHz, DMSO-d₆) δ 7.92 (br s, 1H), 7.89 (d, J=7.8 Hz, 1H), 7.45-7.37 (m, 3H), 7.16 (d, J=8.1 Hz, 1H), 6.67-6.61 (m, 2H), 6.14 (s, 2H), 4.72-4.62 (m, 1H), 3.89-3.81 (m, 1H), 3.74 (s, 3H), 3.72-3.64 (m, 1H), 3.42-3.37 (m, 2H), 3.36-3.34 (m, 1H), 3.29-3.22 (m, 1H), 2.94 (s, 2H), 2.02 (s, 3H), 2.00-1.94 (m, 1H), 1.93-1.86 (m, 1H), 1.68-1.59 (m, 1H), 1.55-1.48 (m, 1H); LCMS: [M+H]⁺=505.50.

### Example 305: 6-(5-(4-((1-acetylpiperidin-4-yl)oxy)-3-methoxyphenyl)-2-amino-6-fluoropyridin-3-yl)-3,4-dihydroisoquinolin-1(2H)-one (I-305)

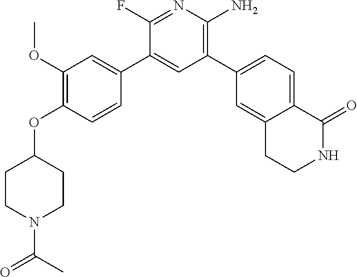 [SMILES: COc1cc(-c2cc(-c3ccc4c(c3)CCNC4=O)c(N)nc2F)ccc1OC1CCN(C(C)=O)CC1]

A procedure similar to Example 191 using 6-(2-amino-6-fluoro-5-(3-methoxy-4-(piperidin-4-yloxy)phenyl)pyridin-3-yl)-3,4-dihydroisoquinolin-1(2H)-one (25 mg, 0.054 mmol) and N,N-diisopropylethylamine (0.047 mL, 0.270 mmol) in CH₂Cl₂ (3 mL) and acetic anhydride 5.11 μl, 0.054 mmol) afforded the title compound (22.8 mg, 79%) as a white powder. ¹H NMR (500 MHz, DMSO-d₆) δ 7.94 (br s, 1H), 7.91 (d, J=7.8 Hz, 1H), 7.65 (d, J=10.1 Hz, 1H), 7.49-7.45 (m, 1H), 7.44 (s, 1H), 7.13 (s, 1H), 7.10-7.03 (m, 2H), 6.24 (s, 2H), 4.56-4.48 (m, 1H), 3.87-3.81 (m, 1H), 3.80 (s, 3H), 3.71-3.64 (m, 1H), 3.44-3.38 (m, 2H), 3.31-3.28 (m, 1H), 3.27-3.20 (m, 1H), 2.95 (t, J=6.5 Hz, 2H), 2.01 (s, 3H), 1.96-1.89 (m, 1H), 1.88-1.81 (m, 1H), 1.67-1.58 (m, 1H), 1.56-1.46 (m, 1H); LCMS: [M+H]⁺=505.50.

### Example 306: 6-(5-(7-((1-acetylpiperidin-4-yl)oxy)benzo[d][1,3]dioxol-4-yl)-2-amino-6-fluoropyridin-3-yl)-3,4-dihydroisoquinolin-1(2H)-one (I-306)

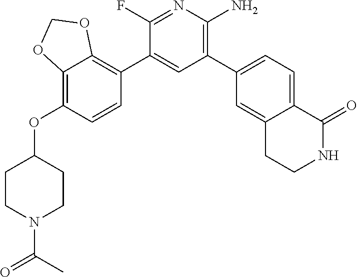 [SMILES: CC(=O)N1CCC(Oc2ccc(-c3cc(-c4ccc5c(c4)CCNC5=O)c(N)nc3F)c3c2OCO3)CC1]

A procedure similar to Example 191 using 6-(2-amino-6-fluoro-5-(7-(piperidin-4-yloxy)benzo[d][1,3]dioxol-4-yl)pyridin-3-yl)-3,4-dihydroisoquinolin-1(2H)-one (20.1 mg, 0.042 mmol) and N,N-diisopropylethylamine (0.037 ml, 0.211 mmol) in CH₂Cl₂ (3 mL) and acetic anhydride (3.99 μL, 0.042 mmol) afforded the title compound (18.6 mg, 81%) as a yellow powder. ¹H NMR (500 MHz, DMSO-d₆) δ 7.94 (br s, 1H), 7.90 (d, J=7.8 Hz, 1H), 7.59 (d, J=9.8 Hz, 1H), 7.44-7.39 (m, 2H), 6.90 (d, J=7.8 Hz, 1H), 6.74 (d, J=8.9 Hz, 1H), 6.30 (s, 2H), 6.00 (s, 2H), 4.68-4.60 (m, 1H), 3.83-3.76 (m, 1H), 3.70-3.61 (m, 1H), 3.43-3.37 (m, 2H), 3.36-3.34 (m, 1H), 3.28-3.23 (m, 1H), 2.98-2.91 (m, 2H), 2.01 (s, 3H), 1.99-1.91 (m, 1H), 1.90-1.82 (m, 1H), 1.68-1.59 (m, 1H), 1.57-1.48 (m, 1H); LCMS: [M+H]⁺=519.57.

### Example 307: 6-(2-amino-5-(4-(4-(cyclopropylmethyl)piperazin-1-yl)phenyl)-6-fluoropyridin-3-yl)-8-fluoro-3,4-dihydroisoquinolin-1(2H)-one (I-307)

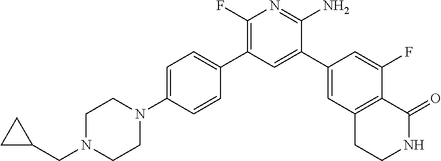 [SMILES: Nc1nc(F)c(-c2ccc(N3CCN(CC4CC4)CC3)cc2)cc1-c1cc(F)c2c(c1)CCNC2=O]

Step 1: tert-butyl 4-(4-(6-amino-2-fluoro-5-(8-fluoro-1-oxo-1,2,3,4-tetrahydroisoquinolin-6-yl)pyridin-3-yl)phenyl)piperazine-1-carboxylate

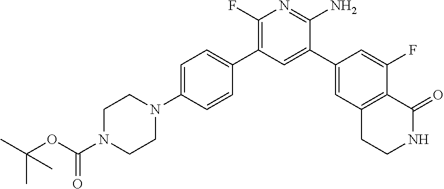 [SMILES: CC(C)(C)OC(=O)N1CCN(c2ccc(-c3cc(-c4cc(F)c5c(c4)CCNC5=O)c(N)nc3F)cc2)CC1]

A procedure similar to Example 177 Step 1 using 6-(2-amino-5-bromo-6-fluoropyridin-3-yl)-8-fluoro-3,4-dihydroisoquinolin-1(2H)-one (130 mg, 0.367 mmol), tert-butyl 4-[4-(4,4,5,5-tetramethyl-1,3,2-dioxaborolan-2-yl)phenyl]tetrahydro-1(2H)-pyrazinecarboxylate (171 mg, 0.440 mmol) and Cs₂CO₃ (299 mg, 0.918 mmol) in DME (5 mL) and water (1.67 mL) and PdCl₂dppf (26.9 mg, 0.037 mmol) afforded the product (197 mg, 100%) as a brown solid. LCMS: [M+H]⁺=536.67.

Step 2: 6-(2-amino-6-fluoro-5-(4-(piperazin-1-yl)phenyl)pyridin-3-yl)-8-fluoro-3,4-dihydroisoquinolin-1(2H)-one

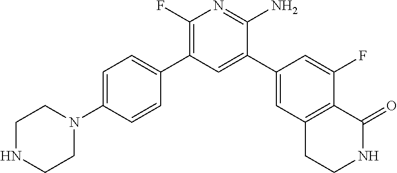 [SMILES: Nc1nc(F)c(-c2ccc(N3CCNCC3)cc2)cc1-c1cc(F)c2c(c1)CCNC2=O]

To a solution of tert-butyl 4-(4-(6-amino-2-fluoro-5-(8-fluoro-1-oxo-1,2,3,4-tetrahydroisoquinolin-6-yl)pyridin-3-yl)phenyl)piperazine-1-carboxylate (196 mg, 0.366 mmol) in CH₂Cl₂ (5.0 mL) was added TFA (0.84 mL, 11.0 mmol). The reaction was stirred at RT for 96 h, then concentrated onto Celite® and purified by reverse phase chromatography (C18, 0-100% ACN/H₂O) to afford the TFA salt of the product (137.1 mg, 68%) as a yellow solid. LCMS: [M+H]⁺=436.51.

Step 3: 6-(2-amino-5-(4-(4-(cyclopropylmethyl)piperazin-1-yl)phenyl)-6-fluoropyridin-3-yl)-8-fluoro-3,4-dihydroisoquinolin-1(2H)-one

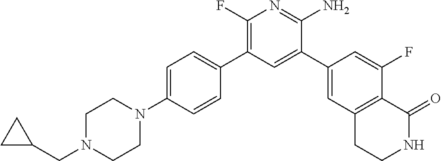 [SMILES: Nc1nc(F)c(-c2ccc(N3CCN(CC4CC4)CC3)cc2)cc1-c1cc(F)c2c(c1)CCNC2=O]

A procedure analogous to Example 186 using 6-(2-amino-6-fluoro-5-(4-(piperazin-1-yl)phenyl)pyridin-3-yl)-8-fluoro-3,4-dihydroisoquinolin-1(2H)-one, trifluoroacetate (25 mg, 0.045 mmol), N,N-diisopropylethylamine (0.079 mL, 0.455 mmol) in DMF (1.0 mL), and (bromomethyl)cyclopropane (30.7 mg, 0.227 mmol) afforded the title compound (14.6 mg, 66%) as a white solid. ¹H NMR (500 MHz, DMSO-d₆) δ 7.88 (br s, 1H), 7.52 (d, J=10.2 Hz, 1H), 7.30 (br d, J=8.1 Hz, 2H), 7.19 (s, 1H), 7.15 (br d, J=12.2 Hz, 1H), 6.87 (d, J=8.8 Hz, 2H), 6.18 (s, 2H), 3.03-3.11 (m, 4H), 2.83 (br t, J=6.2 Hz, 2H), 2.44-2.51 (m, 4H), 2.12 (d, J=6.6 Hz, 2H), 0.71-0.80 (m, 1H), 0.33-0.42 (m, 2H), 0.00 (q, J=4.7 Hz, 2H); LCMS: [M+H]⁺=490.60.

### Example 308: 6-(2-amino-5-(4-(1-(cyclopropylmethyl)piperidin-4-yl)phenyl)-6-fluoropyridin-3-yl)-8-fluoro-3,4-dihydroisoquinolin-1(2H)-one (I-308)

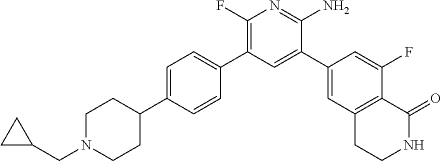 [SMILES: Nc1nc(F)c(-c2ccc(C3CCN(CC4CC4)CC3)cc2)cc1-c1cc(F)c2c(c1)CCNC2=O]

A procedure similar to Example 186 using 6-(2-amino-6-fluoro-5-(4-(piperidin-4-yl)phenyl)pyridin-3-yl)-8-fluoro-3,4-dihydroisoquinolin-1(2H)-one, trifluoroacetate (25 mg, 0.046 mmol) and N,N-diisopropylethylamine (0.079 mL, 0.456 mmol) in DMF (1.0 ml) and bromomethyl)cyclopropane (30.8 mg, 0.228 mmol) afforded the title compound (6.28 mg, 28%) as a white solid. ¹H NMR (500 MHz, DMSO-d₆) δ 7.86 (br s, 1H), 7.54 (d, J=10.0 Hz, 1H), 7.36 (br d, J=7.6 Hz, 2H), 7.08-7.22 (m, 4H), 6.27 (s, 2H), br s, 2H), 2.80 (br t, J=6.1 Hz, 2H), 2.21 (br d, J=17.6 Hz, 2H), 1.88-2.07 (m, 1H), 1.54-1.72 (m, 4H), 0.76 (br s, 1H), 0.37 (br d, J=7.0 Hz, 2H), 0.00 (br s, 2H); LCMS: [M+H]⁺=489.66.

### Example 309: 6-(2-amino-6-fluoro-5-(4-morpholinophenyl)pyridin-3-yl)-8-fluoro-3,4-dihydroisoquinolin-1(2H)-one (I-309)

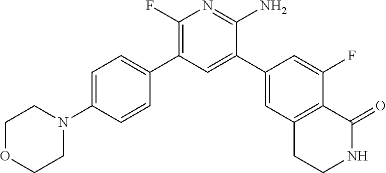 [SMILES: Nc1nc(F)c(-c2ccc(N3CCOCC3)cc2)cc1-c1cc(F)c2c(c1)CCNC2=O]

A procedure analogous to Example 177, Step 1 using 6-(2-amino-5-bromo-6-fluoropyridin-3-yl)-8-fluoro-3,4-dihydroisoquinolin-1(2H)-one (35 mg, 0.099 mmol), 4-(morpholino)phenylboronic acid (24.55 mg, 0.119 mmol) Cs₂CO₃ (80 mg, 0.247 mmol) DME (2 mL), water (0.67 mL) and PdCl₂dppf (7.23 mg, 9.88 μmol) afforded the title compound (13.7 mg, 32%) as a beige solid. ¹H NMR (500 MHz, DMSO-d₆) δ 8.00 (br s, 1H), 7.64 (br d, J=9.9 Hz, 1H), 7.44 (br d, J=8.1 Hz, 2H), 7.30 (s, 1H), 7.27 (br d, J=12.1 Hz, 1H), 6.99 (br d, J=8.4 Hz, 2H), 6.31 (br s, 2H), 3.75 (br s, 4H), 3.14 (br s, 4H), 2.94 (br d, J=5.5 Hz, 2H); LCMS: [M+H]⁺=437.52.

### Example 310: 6-(2-amino-6-fluoro-5-(4-(4-isopropylpiperazin-1-yl)phenyl)pyridin-3-yl)-3-((dimethylamino)methyl)-3,4-dihydroisoquinolin-1(2H)-one (I-310)

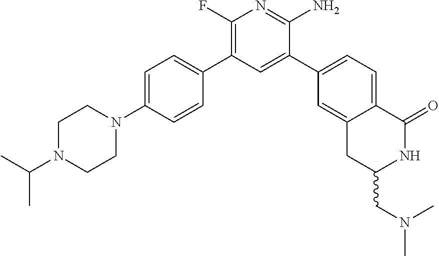 [SMILES: CC(C)N1CCN(c2ccc(-c3cc(-c4ccc5c(c4)CC(CN(C)C)NC5=O)c(N)nc3F)cc2)CC1]

Step 1: 6-bromo-3-((dimethylamino)methyl)-3,4-dihydroisoquinolin-1(2H)-one

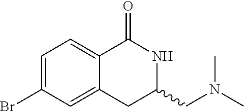 [SMILES: CN(C)CC1Cc2cc(Br)ccc2C(=O)N1]

A mixture of 6-bromo-3-((methylamino)methyl)-3,4-dihydroisoquinolin-1(2H)-one (150 mg, 0.557 mmol), formaldehyde solution, 37% wt in water (0.124 mL, 1.67 mmol) was stirred overnight at RT with NaBH(OAc)₃ (472 mg, 2.23 mmol) and glacial AcOH (0.016 mL, 0.279 mmol). The reaction mixture was partitioned between CH₂Cl₂ (5 mL) and 1N NaOH (aq) solution (7 mL). The layers were separated and the aqueous layer was further extracted with CH₂Cl₂ (5 mL). The combined organic extract was washed successively with water (5 mL), brine (5 mL), dried over Na₂SO₄ and concentrated to yield the title compound as an off white solid (154 mg, 98%). LCMS: [M+H]⁺=283.31.

Step 2: 3-((dimethylamino)methyl)-6-(4,4,5,5-tetramethyl-1,3,2-dioxaborolan-2-yl)-3,4-dihydroisoquinolin-1(2H)-one

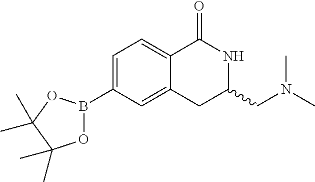

A procedure analogous to Example 108, Step 2 using 6-bromo-3-((dimethylamino)methyl)-3,4-dihydroisoquinolin-1(2H)-one (151 mg, 0.533 mmol), bis(pinacolato)diboron (153 mg, 0.603 mmol), [1,12-bis(diphenylphosphino)ferrocene]dichloropalladium(II) (39.0 mg, 0.053 mmol) and KOAc (157 mg, 1.60 mmol) in 1,4-dioxane (10 mL) at 100° C. for 1 h afforded the crude product which was filtered through a thin pad of Celite, the filter cake was washed in with EtOAc and combined filtrate was concentrated to collect the crude product as a brown (176 mg, quantitative yield assuming 100% conversion). This was taken to the next step without any purification. LCMS: [M+H]⁺=331.64.

Step 3: 6-(2-amino-5-bromo-6-fluoropyridin-3-yl)-3-((dimethylamino)methyl)-3,4-dihydroisoquinolin-1(2H)-one

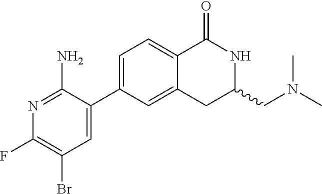 [SMILES: CN(C)CC1Cc2cc(-c3cc(Br)c(F)nc3N)ccc2C(=O)N1]

A procedure analogous to that of Example 52, Step 1, by heating under microwave reaction conditions at 80° C. for 1 h, 5-bromo-6-fluoro-3-iodopyridin-2-amine (199 mg, 0.627 mmol), 3-((dimethylamino)methyl)-6-(4,4,5,5-tetramethyl-1,3,2-dioxaborolan-2-yl)-3,4-dihydroisoquinolin-1(2H)-one (176 mg, 0.533 mmol), Na₂CO₃ (163 mg, 1.32 mmol), bis(triphenylphosphine)palladium(II) dichloride in CH₃CN (7 mL) and water (2.5 mL), afforded the crude product. The reaction mixture was concentrated onto Celite® and silica gel chromatography (eluting with CH₂Cl₂ containing 0-2.5% MeOH and 0-0.25% NH₄OH) afforded the product as a beige solid (134.5 mg, 55% yield). LCMS: [M+H]⁺=393.45.

Step 4: 6-(2-amino-6-fluoro-5-(4-(4-isopropylpiperazin-1-yl)phenyl)pyridin-3-yl)-3-((dimethylamino)methyl)-3,4-dihydroisoquinolin-1(2H)-one

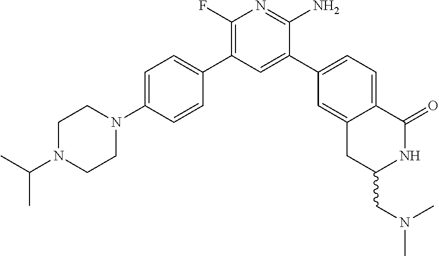 [SMILES: CC(C)N1CCN(c2ccc(-c3cc(-c4ccc5c(c4)CC(CN(C)C)NC5=O)c(N)nc3F)cc2)CC1]

A procedure analogous to Example 177, Step 1 using 6-(2-amino-5-bromo-6-fluoropyridin-3-yl)-3-((dimethylamino)methyl)-3,4-dihydroisoquinolin-1(2H)-one (30 mg, 0.076 mmol)), 4-(4-isopropylpiperazinyl)phenylboronic acid, pinacol ester (35.3 mg, 0.107 mmol), [1,12-bis(diphenylphosphino)ferrocene]dichloropalladium(II) (8.37 mg, 0.011 mmol), Cs₂CO₃ (74.6 mg, 0.229 mmol), degassed DME (4 mL) and water (1 mL) afforded the title compound as a beige solid (11 mg, 27%). ¹H NMR (500 MHz, DMSO-d₆) δ 7.91 (d, J=7.8 Hz, 1H), 7.63-7.55 (m, 1H), 7.51-7.37 (m, 5H), 7.02-6.91 (m, 2H), 6.23-6.08 (m, 2H), 3.77-3.75 (m, 1H), 3.18-3.14 (m, 4H), 3.10-3.05 (m, 1H), 2.86-2.79 (m, 1H), 2.72-2.67 (m, 1H), 2.62-2.56 (m, 4H), 2.43-2.37 (m, 1H), 2.35-2.30 (m, 1H), 2.22-2.15 (m, 6H), 1.05-0.97 (m, 6H); LCMS: [M+H]⁺=517.68.

### Example 311: 6-(2-amino-6-fluoro-5-(4-morpholinophenyl)pyridin-3-yl)-3-((dimethylamino)methyl)-3,4-dihydroisoquinolin-1(2H)-one (I-311)

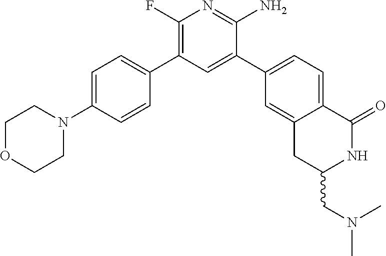 [SMILES: CN(C)CC1Cc2cc(-c3cc(-c4ccc(N5CCOCC5)cc4)c(F)nc3N)ccc2C(=O)N1]

A procedure analogous to that of Example 177 Step 1 using 6-(2-amino-5-bromo-6-fluoropyridin-3-yl)-3-((dimethylamino)methyl)-3,4-dihydroisoquinolin-1(2H)-one (30 mg, 0.076 mmol), 4-(morpholino)phenylboronic acid (22.11 mg, 0.107 mmol), [1,12-bis(diphenylphosphino)ferrocene]dichloropalladium(II) (8.37 mg, 0.011 mmol), Cs₂CO₃ (74.6 mg, 0.229 mmol), DME (4 mL) and water (1 mL) afforded the crude product which was concentrated onto Celite®. Silica gel chromatography (eluting with DCM containing 0-2% MeOH and 0-0.2% NH₄OH), followed by reverse phase chromatography (C18; 0-40% CH₃CN/water) afforded the title compound as a beige solid (9.5 mg, 25%). ¹H NMR (500 MHz, DMSO-d₆) δ 7.93-7.86 (m, 1H), 7.62-7.57 (m, 1H), 7.50-7.41 (m, 5H), 7.02-6.97 (m, 2H), 6.18 (s, 2H), 3.77-3.71 (m, 5H), 3.15-3.11 (m, 4H), 3.09-3.03 (m, 1H), 2.84-2.77 (m, 1H), 2.43-2.36 (m, 1H), 2.34-2.28 (m, 1H), 2.18 (s, 6H); LCMS: [M+H]⁺=476.69.

### Example 312: 6-(2-amino-5-(4-chlorophenyl)-6-fluoropyridin-3-yl)-3-((dimethylamino)methyl)-3,4-dihydroisoquinolin-1(2H)-one (I-312)

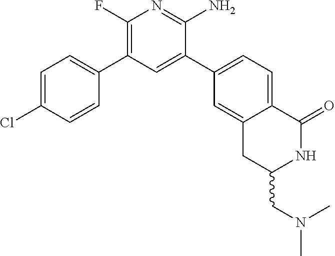 [SMILES: CN(C)CC1Cc2cc(-c3cc(-c4ccc(Cl)cc4)c(F)nc3N)ccc2C(=O)N1]

A procedure analogous to that of Example 177, Step 1 with 6-(2-amino-5-bromo-6-fluoropyridin-3-yl)-3-((dimethylamino)methyl)-3,4-dihydroisoquinolin-1(2H)-one (30 mg, 0.076 mmol), 4-chlorophenylboronic acid, pinacol ester (25.5 mg, 0.107 mmol), [1,12-bis(diphenylphosphino)ferrocene]dichloropalladium(II) (8.37 mg, 0.011 mmol) and Cs₂CO₃ (74.6 mg, 0.229 mmol), DME (4 mL) and water (1 mL) was used. The reaction mixture was concentrated onto Celite®. Silica gel chromatography (eluting with 0-70% EtOAc/hexanes), followed by reverse phase chromatography (C18; 0-40% acetonitrile/water) afforded the title compound as a beige solid (14 mg, 41%). ¹H NMR (500 MHz, DMSO-d₆) δ 7.97-7.87 (m, 1H), 7.72-7.65 (m, 1H), 7.63-7.58 (m, 2H), 7.51-7.44 (m, 5H), 6.47-6.35 (m, 2H), 3.79-3.72 (m, 1H), 3.10-3.02 (m, 1H), 2.87-2.77 (m, 1H), 2.44-2.37 (m, 1H), 2.35-2.29 (m, 1H), 2.19 (s, 6H); LCMS: [M+H]⁺=425.61.

### Example 313: 6-(6-amino-2-fluoro-2′-methyl-[3,4′-bipyridin]-5-yl)-7-fluoro-3,4-dihydroisoquinolin-1(2H)-one (I-313)

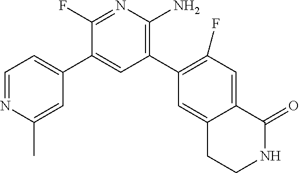 [SMILES: Cc1cc(-c2cc(-c3cc4c(cc3F)C(=O)NCC4)c(N)nc2F)ccn1]

A procedure similar to Example 177, Step 1 using (2-methylpyridine-4-yl) boronic acid (26.4 mg, 0.193 mmol), 6-(2-amino-5-bromo-6-fluoropyridin-3-yl)-7-fluoro-3,4-dihydroisoquinolin-1(2H)-one (62 mg, 0.175 mmol), 1,4-dioxane (3 mL), Cs₂CO₃ (171 mg, 0.525 mmol) in water (1 mL) and Pd(dppf)Cl₂ (19.21 mg, 0.026 mmol) afforded the title compound (41.0 mg, 61%) as a beige powder. ¹H NMR (500 MHz, DMSO-d₆) δ 8.42 (d, J=5.3 Hz, 1H), 8.12 (br s, 1H), 7.82 (d, J=10.1 Hz, 1H), 7.61 (d, J=10.1 Hz, 1H), 7.47 (s, 1H), 7.43 (d, J=7.0 Hz, 1H), 7.38 (br d, J=5.1 Hz, 1H), 6.59 (br s, 2H), 3.41 (dt, J=2.7, 6.5 Hz, 2H), 2.92 (br t, J=6.4 Hz, 2H), 2.48 (s, 3H); LCMS: [M+H]⁺=367.21.

### Example 314: 6-(6-amino-2′-(cyclopropylmethoxy)-2-fluoro-[3,4′-bipyridin]-5-yl)-7-fluoro-3,4-dihydroisoquinolin-1(2H)-one (I-314)

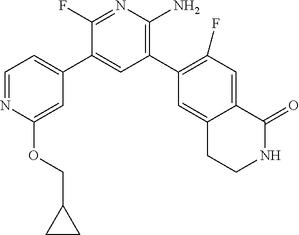 [SMILES: Nc1nc(F)c(-c2ccnc(OCC3CC3)c2)cc1-c1cc2c(cc1F)C(=O)NCC2]

A procedure similar to Example 177, Step 1 using 2-(cyclopropylmethoxy)-4-(4,4,5,5-tetramethyl-1,3,2-dioxaborolan-2-yl)pyridine (52.1 mg, 0.189 mmol), 6-(2-amino-5-bromo-6-fluoropyridin-3-yl)-7-fluoro-3,4-dihydroisoquinolin-1(2H)-one (61 mg, 0.172 mmol), 1,4-dioxane (3 ml), Cs₂CO₃ (168 mg, 0.517 mmol) in water (1 mL) and Pd(dppf)Cl₂ (18.90 mg, 0.026 mmol) afforded the title compound (54.4 mg, 71%) as a beige powder. ¹H NMR (500 MHz, DMSO-d₆) δ 8.11 (d, J=5.3 Hz, 2H), 7.82 (d, J=10.0 Hz, 1H), 7.61 (d, J=10.0 Hz, 1H), 7.44 (d, J=7.1 Hz, 1H), 7.17 (d, J=5.4 Hz, 1H), 6.98 (s, 1H), 6.59 (br s, 2H), 4.10 (d, J=7.1 Hz, 2H), 3.41 (dt, J=6.5, 2.8 Hz, 2H), 2.92 (br t, J=6.5 Hz, 2H), 1.29-1.19 (m, 1H), 0.58-0.50 (m, 2H), 0.32 (br dd, J=1.3, 4.8 Hz, 2H); LCMS: [M+H]⁺=423.11.

### Example 315: 6-(2-amino-5-(3-(cyclopropylmethoxy)phenyl)-6-fluoropyridin-3-yl)-7-fluoro-3,4-dihydroisoquinolin-1(2H)-one (I-315)

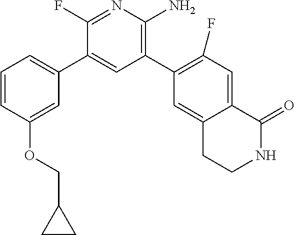 [SMILES: Nc1nc(F)c(-c2cccc(OCC3CC3)c2)cc1-c1cc2c(cc1F)C(=O)NCC2]

A procedure similar to Example 177, Step 1 using 6-(2-amino-5-bromo-6-fluoropyridin-3-yl)-7-fluoro-3,4-dihydroisoquinolin-1(2H)-one (63 mg, 0.178 mmol), 3-(cyclopropylmethoxy)phenylboronic acid (37.6 mg, 0.196 mmol), 1,4-dioxane (3 mL) Cs₂CO₃ (174 mg, 0.534 mmol) in water (1 mL) and [1,12-bis(diphenylphosphino)ferrocene]dichloropalladium(II) (19.52 mg, 0.027 mmol) afforded the title compound (26.7 mg, 34%) as a white powder. ¹H NMR (500 MHz, DMSO-d₆) δ 8.11 (br s, 1H), 7.67 (d, J=10.1 Hz, 1H), 7.61 (d, J=10.1 Hz, 1H), 7.43 (d, J=7.1 Hz, 1H), 7.30 (t, J=7.9 Hz, 1H), 7.08 (br d, J=7.5 Hz, 1H), 7.04 (s, 1H), 6.86 (dd, J=8.1, 2.1 Hz, 1H), 6.32 (s, 2H), 3.84 (d, J=7.0 Hz, 2H), 3.45-3.37 (m, 2H), 2.92 (br t, J=6.5 Hz, 2H), 1.26-1.16 (m, 1H), 0.59-0.51 (m, 2H), 0.35-0.27 (m, 2H); LCMS: [M+H]⁺=422.44.

### Example 316: 6-(2-amino-5-(4-(cyclopropylmethoxy)phenyl)-6-fluoropyridin-3-yl)-7-fluoro-3,4-dihydroisoquinolin-1(2H)-one (I-316)

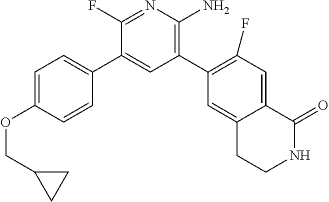 [SMILES: Nc1nc(F)c(-c2ccc(OCC3CC3)cc2)cc1-c1cc2c(cc1F)C(=O)NCC2]

A procedure analogous to Example 177, Step 1 using 4-(cyclopropylmethoxy)phenylboronic acid (37.0 mg, 0.193 mmol), 6-(2-amino-5-bromo-6-fluoropyridin-3-yl)-7-fluoro-3,4-dihydroisoquinolin-1(2H)-one (62 mg, 0.175 mmol), 1,4-dioxane (3 mL) and Cs₂CO₃ (171 mg, 0.525 mmol) in water (1 mL) and Pd(dppf)Cl₂ (19.21 mg, 0.026 mmol) afforded the title compound (16.0 mg, 21%) as a beige powder. ¹H NMR (500 MHz, DMSO-d₆) δ 8.11 (br s, 1H), 7.60 (dd, J=6.8, 10.1 Hz, 2H), 7.47-7.39 (m, 3H), 6.96 (d, J=8.8 Hz, 2H), 6.23 (s, 2H), 3.83 (d, J=7.0 Hz, 2H), 3.44-3.37 (m, 2H), 2.96-2.88 (m, 2H), 1.26-1.17 (m, 1H), 0.60-0.53 (m, 2H), 0.35-0.29 (m, 2H); LCMS: [M+H]⁺=422.44.

### Example 317: 6-(2-amino-5-(4-((1-(2-cyclopropylacetyl)azetidin-3-yl)oxy)phenyl)-6-fluoropyridin-3-yl)-3,4-dihydroisoquinolin-1(2H)-one (I-317)

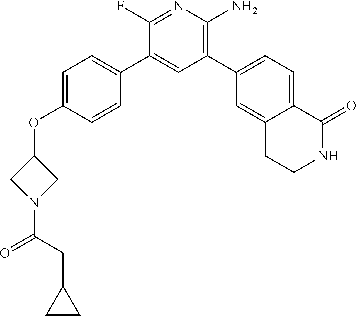 [SMILES: Nc1nc(F)c(-c2ccc(OC3CN(C(=O)CC4CC4)C3)cc2)cc1-c1ccc2c(c1)CCNC2=O]

A procedure analogous to Example 191 using 6-(2-amino-5-(4-(azetidin-3-yloxy)phenyl)-6-fluoropyridin-3-yl)-3,4-dihydroisoquinolin-1(2H)-one (24.8 mg, 0.061 mmol) in CH₂Cl₂ (1 mL) was added N,N-diisopropylethylamine (0.053 mL, 0.307 mmol) at RT followed by 2-cyclopropylacetyl chloride (8.72 mg, 0.074 mmol) afforded the title compound (19.3 mg, 62%) as a white powder. ¹H NMR (500 MHz, DMSO-d₆) δ 7.94 (br s, 1H), 7.90 (d, J=7.9 Hz, 1H), 7.60 (d, J=10.1 Hz, 1H), 7.52-7.43 (m, 4H), 6.90 (d, J=8.8 Hz, 2H), 6.26 (s, 2H), 5.10-5.01 (m, 1H), 4.62-4.51 (m, 1H), 4.36-4.26 (m, 1H), 4.12-4.04 (m, 1H), 3.82-3.75 (m, 1H), 3.40 (br d, J=2.4 Hz, 2H), 2.95 (s, 2H), 2.03 (d, J=6.8 Hz, 2H), 0.98-0.87 (m, 1H), 0.48-0.38 (m, 2H), 0.13-0.05 (m, 2H); LCMS: [M+H]⁺=487.52.

### Example 318: 6-(2-amino-6-fluoro-5-(4-((1-(oxetan-3-yl)azetidin-3-yl)oxy)phenyl)pyridin-3-yl)-3,4-dihydroisoquinolin-1(2H)-one (I-318)

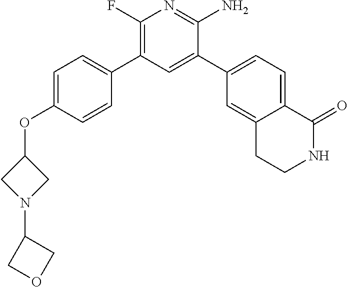 [SMILES: Nc1nc(F)c(-c2ccc(OC3CN(C4COC4)C3)cc2)cc1-c1ccc2c(c1)CCNC2=O]

A procedure similar to Example 218 using 6-(2-amino-5-(4-(azetidin-3-yloxy)phenyl)-6-fluoropyridin-3-yl)-3,4-dihydroisoquinolin-1(2H)-one (33.7 mg, 0.083 mmol) in CH₂Cl₂ (1 mL), oxetan-3-one (45 mg, 0.624 mmol) and 2 drops glacial AcOH (40 mg, 0.666 mmol) afforded the title compound (13.8 mg, 34%) as a white powder. ¹H NMR (500 MHz, DMSO-d₆) δ 7.94 (br s, 1H), 7.90 (d, J=7.8 Hz, 1H), 7.59 (d, J=10.3 Hz, 1H), 7.51-7.41 (m, 4H), 6.89 (d, J=8.8 Hz, 2H), 6.24 (s, 2H), 4.93-4.84 (m, 1H), 4.57 (s, 2H), 4.40-4.32 (m, 2H), 3.83-3.71 (m, 3H), 3.44-3.38 (m, 3H), 3.19-3.12 (m, 2H), 2.97-2.92; (m, 2H); LCMS: [M+H]⁺=461.49.

### Example 319: 6-(2-amino-5-(4-((3R,5S)-3,5-dimethylpiperazin-1-yl)phenyl)-6-fluoropyridin-3-yl)-3,4-dihydroisoquinolin-1(2H)-one (I-319)

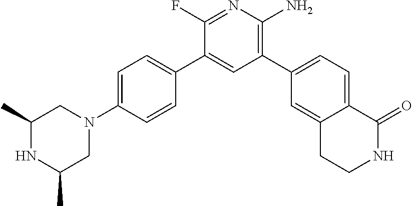 [SMILES: C[C@@H]1CN(c2ccc(-c3cc(-c4ccc5c(c4)CCNC5=O)c(N)nc3F)cc2)C[C@H](C)N1]

Step 1: tert-butyl (2S,6R)-4-(4-bromophenyl)-2,6-dimethylpiperazine-1-carboxylate

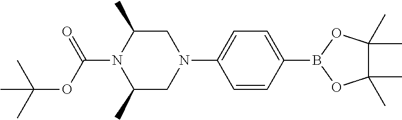 [SMILES: C[C@@H]1CN(c2ccc(B3OC(C)(C)C(C)(C)O3)cc2)C[C@H](C)N1C(=O)OC(C)(C)C]

A procedure similar to Example 108 Step 1 using 1-bromo-4-iodobenzene (560 mg, 1.98 mmol), cis-1-Boc-2,6-dimethyl-piperazine (636 mg, 2.97 mmol), tris(dibenzylideneacetone)dipalladium (0) (54.4 mg, 0.059 mmol), 4,5-bis(diphenylphosphino)-9,9-dimethylxanthene (103 mg, 0.178 mmol) and Cs₂CO₃ (1935 mg, 5.94 mmol) afforded the product (269 mg, 37%) as a yellow oil. LCMS: [M+H]⁺=369.39.

Step 2: tert-butyl (2S,6R)-4-(4-(6-amino-2-fluoro-5-(1-oxo-1,2,3,4-tetrahydroisoquinolin-6-yl)pyridin-3-yl)phenyl)-2,6-dimethylpiperazine-1-carboxylate

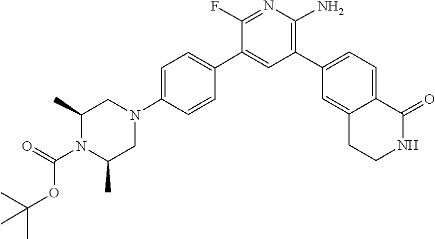 [SMILES: C[C@@H]1CN(c2ccc(-c3cc(-c4ccc5c(c4)CCNC5=O)c(N)nc3F)cc2)C[C@H](C)N1C(=O)OC(C)(C)C]

A procedure similar to Example 43, Step 5 using 6-(2-amino-6-fluoro-5-(4,4,5,5-tetramethyl-1,3,2-dioxaborolan-2-yl)pyridin-3-yl)-3,4-dihydroisoquinolin-1(2H)-one (116 mg, 0.302 mmol), tert-butyl (2S,6R)-4-(4-bromophenyl)-2,6-dimethylpiperazine-1-carboxylate (93 mg, 0.252 mmol), [1,12-bis(diphenylphosphino)ferrocene]dichloropalladium(II) (18.43 mg, 0.025 mmol), aqueous K₃PO₄ (0.387 mL of a 1.3 M solution, 0.504 mmol), DME (4 mL) and water (2 mL) afforded the product (80 mg, 58%)¹H NMR (500 MHz, DMSO-d₆) δ 7.93 (br s, 1H), 7.90 (d, J=7.8 Hz, 1H), 7.58 (d, J=10.1 Hz, 1H), 7.48-7.39 (m, 4H), 6.99 (d, J=8.9 Hz, 2H), 6.18 (s, 2H), 4.15-4.08 (m, 2H), 3.54 (br d, J=12.2 Hz, 2H), 3.40 (td, J=6.5, 2.7 Hz, 2H), 2.95 (t, J=6.5 Hz, 2H), 2.81 (dd, J=12.2, 4.2 Hz, 2H), 1.43 (s, 9H), 1.25 (d, J=6.8 Hz, 6H); LCMS: [M +H]⁺=546.58.

Step 3: 6-(2-amino-5-(4-((3R,5S)-3,5-dimethylpiperazin-1-yl)phenyl)-6-fluoropyridin-3-yl)-3,4-dihydroisoquinolin-1(2H)-one

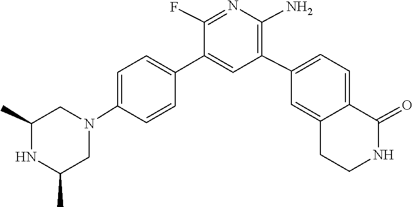 [SMILES: C[C@@H]1CN(c2ccc(-c3cc(-c4ccc5c(c4)CCNC5=O)c(N)nc3F)cc2)C[C@H](C)N1]

In a vial was placed tert-butyl 4-(4-(6-amino-2-fluoro-5-(1-oxo-1,2,3,4-tetrahydroisoquinolin-6-yl)pyridin-3-yl)phenyl)-cis-2,6-dimethylpiperazine-1-carboxylate (80 mg, 0.147 mmol), CH₂Cl₂ (5 mL) and TFA (2 mL, 0.147 mmol). The flask was mixed by vortexing and then allowed to sit for 2 h, when LCMS indicated complete conversion. The reaction was concentrated and then purified by loading onto a column containing PoraPak RxnCX acidic PE resin, washing (MeOH) and then eluting off the compound with 3% NH₄OH in MeOH to provide the title compound (54 mg, 82%) as a tan solid. ¹H NMR (500 MHz, DMSO-d₆) δ 7.93 (br s, 1H), 7.90 (d, J=7.9 Hz, 1H), 7.57 (d, J=10.1 Hz, 1H), 7.48-7.44 (m, 1H), 7.44 (s, 1H), 7.38 (d, J=7.8 Hz, 2H), 6.95 (d, J=8.9 Hz, 2H), 6.15 (s, 2H), 3.56 (dd, J=11.4, 2.1 Hz, 2H), 3.42-3.38 (m, 3H), 2.95 (t, J=6.5 Hz, 2H), 2.87-2.79 (m, 2H), 2.12 (t, J=11.0 Hz, 2H), 1.02 (d, J=6.2 Hz, 6H); LCMS: [M+H]⁺=446.36.

### Example 320: 6-(2-amino-5-(4-((1S,5R)-3-(cyclopropylmethyl)-3-azabicyclo[3.1.0]hexan-1-yl)phenyl)-6-fluoropyridin-3-yl)-8-fluoro-3,4-dihydroisoquinolin-1(2H)-one formate (I-320)

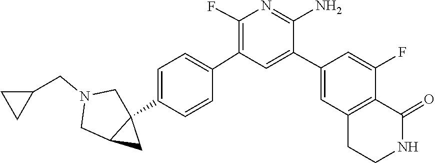 [SMILES: Nc1nc(F)c(-c2ccc([C@]34C[C@H]3CN(CC3CC3)C4)cc2)cc1-c1cc(F)c2c(c1)CCNC2=O]

Step 1: (1S,5R)-1-(4-(4,4,5,5-tetramethyl-1,3,2-dioxaborolan-2-yl)phenyl)-3-azabicyclo[3.1.0]hexane

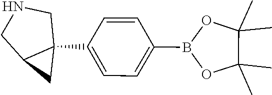 [SMILES: CC1(C)OB(c2ccc([C@@]34CNC[C@@H]3C4)cc2)OC1(C)C]

To a microwave vial charged with (1 S,5R)-1-(4-bromophenyl)-3-azabicyclo[3.1.0]hexane (130 mg, 0.546 mmol), bis(pinacolato)diboron (152 mg, 0.601 mmol) and KOAc (161 mg, 1.638 mmol) was added anhydrous 1,4-dioxane (6.0 mL). The system was degassed then PdCl₂dppf (39.9 mg, 0.055 mmol) was added. The mixture was flushed with nitrogen then heated at 100° C. 3 h. The reaction was diluted with acetonitrile, filtered through a pad of Celite, concentrated in vacuo and used as-is the next step assuming full conversion. LCMS: [M+H]⁺=286.45.

Step 2: 6-(5-(4-((1S,5R)-3-azabicyclo[3.1.0]hexan-1-yl)phenyl)-2-amino-6-fluoropyridin-3-yl)-8-fluoro-3,4-dihydroisoquinolin-1(2H)-one

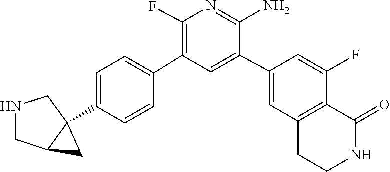 [SMILES: Nc1nc(F)c(-c2ccc([C@@]34CNC[C@@H]3C4)cc2)cc1-c1cc(F)c2c(c1)CCNC2=O]

A procedure similar to Example 246 using 6-(2-amino-5-bromo-6-fluoropyridin-3-yl)-8-fluoro-3,4-dihydroisoquinolin-1(2H)-one (130 mg, 0.367 mmol), (1 S,5R)-1-(4-(4,4,5,5-tetramethyl-1,3,2-dioxaborolan-2-yl)phenyl)-3-azabicyclo[3.1.0]hexane (136 mg, 0.477 mmol) and Cs₂CO₃ (299 mg, 0.918 mmol), DME (5 mL), water (1.67 mL) and PdCl₂dppf (26.9 mg, 0.037 mmol) afforded the title compound (58 mg, 37%) as a brown solid. LCMS: [M+H]⁺=433.57.

### Example 321: 6-(2-amino-5-(4-((1-(cyclopropylmethyl)azetidin-3-yl)oxy)phenyl)-6-fluoropyridin-3-yl)-3,4-dihydroisoquinolin-1(2H)-one (I-321)

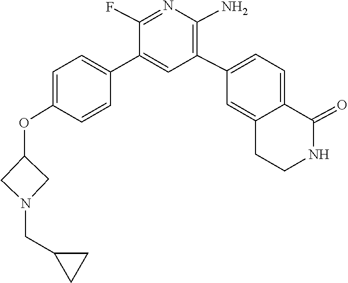 [SMILES: Nc1nc(F)c(-c2ccc(OC3CN(CC4CC4)C3)cc2)cc1-c1ccc2c(c1)CCNC2=O]

A procedure similar to Example 186 using 6-(2-amino-5-(4-(azetidin-3-yloxy)phenyl)-6-fluoropyridin-3-yl)-3,4-dihydroisoquinolin-1(2H)-one (27.1 mg, 0.067 mmol) in DMF (1 mL), N,N-diisopropylethylamine (0.070 mL, 0.402 mmol) and (bromomethyl)cyclopropane (18.09 mg, 0.134 mmol) afforded the title compound (13.4 mg, 41%) as a beige powder. ¹H NMR (500 MHz, DMSO-d₆) δ 7.94 (br s, 1H), 7.90 (d, J=7.9 Hz, 1H), 7.59 (d, J=10.1 Hz, 1H), 7.49-7.42 (m, 4H), 6.88 (d, J=8.8 Hz, 2H), 6.24 (s, 2H), 4.87-4.76 (m, 1H), 3.86-3.74 (m, 2H), 3.43-3.37 (m, 2H), 3.08-2.98 (m, 2H), 2.97-2.91 (m, 2H), 2.37-2.29 (m, 2H), 0.79-0.70 (m, 1H), 0.43-0.34 (m, 2H), 0.14-0.05 (m, 2H); LCMS: [M+H]⁺=459.53.

### Example 322: 6-(2-amino-5-(4-((1-(cyclopropylmethyl)piperidin-4-yl)oxy)phenyl)-6-fluoropyridin-3-yl)-3,4-dihydroisoquinolin-1(2H)-one (I-322)

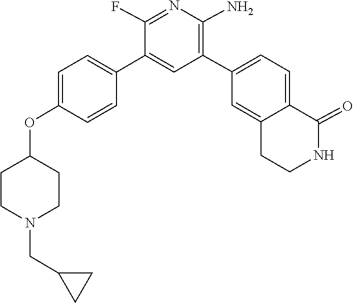 [SMILES: Nc1nc(F)c(-c2ccc(OC3CCN(CC4CC4)CC3)cc2)cc1-c1ccc2c(c1)CCNC2=O]

A procedure similar to Example 186 using 6-(2-amino-6-fluoro-5-(4-(piperidin-4-yloxy)phenyl)pyridin-3-yl)-3,4-dihydroisoquinolin-1(2H)-one (24.7 mg, 0.057 mmol) in DMF (1 mL), N,N-diisopropylethylamine (0.060 mL, 0.343 mmol) and (bromomethyl)cyclopropane (15.42 mg, 0.114 mmol) afforded the title compound (14.5 mg, 50%) as a white powder. ¹H NMR (500 MHz, DMSO-d₆) δ 7.94 (br s, 1H), 7.90 (d, J=7.9 Hz, 1H), 7.59 (d, J=10.1 Hz, 1H), 7.49-7.42 (m, 4H), 6.99 (d, J=8.7 Hz, 2H), 6.22 (s, 2H), 4.44-4.34 (m, 1H), 3.45-3.38 (m, 2H), 2.95 (br t, J=6.5 Hz, 2H), 2.84-2.74 (m, 2H), 2.31-2.22 (m, 2H), 2.19 (br d, J=6.2 Hz, 2H), 2.01-1.90 (m, 2H), 1.70-1.57 (m, 2H), 0.89-0.79 (m, 1H), 0.50-0.42 (m, 2H), 0.07 (q, J=4.9 Hz, 2H); LCMS: [M+H]⁺=487.60.

### Example 323: 6-(2-amino-5-(4-((1-(2-cyclopropylacetyl)piperidin-4-yl)oxy)phenyl)-6-fluoropyridin-3-yl)-7-fluoro-3,4-dihydroisoquinolin-1(2H)-one (I-323)

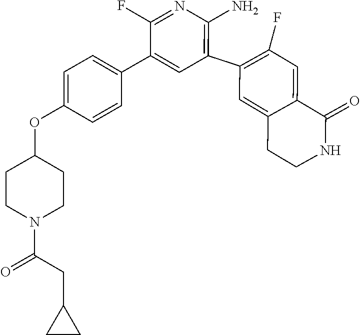 [SMILES: Nc1nc(F)c(-c2ccc(OC3CCN(C(=O)CC4CC4)CC3)cc2)cc1-c1cc2c(cc1F)C(=O)NCC2]

Step 1: tert-butyl 4-(4-(6-amino-2-fluoro-5-(7-fluoro-1-oxo-1,2,3,4-tetrahydroisoquinolin-6-yl)pyridin-3-yl)phenoxy)piperidine-1-carboxylate

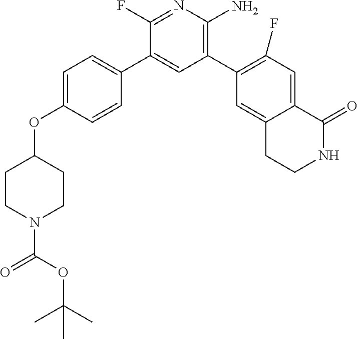 [SMILES: CC(C)(C)OC(=O)N1CCC(Oc2ccc(-c3cc(-c4cc5c(cc4F)C(=O)NCC5)c(N)nc3F)cc2)CC1]

A procedure similar to Example 177, Step 1 using 6-(2-amino-5-bromo-6-fluoropyridin-3-yl)-7-fluoro-3,4-dihydroisoquinolin-1(2H)-one (842 mg, 2.378 mmol), tert-butyl 4-[4-(tetramethyl-1,3,2-dioxaborolan-2-yl)phenoxy]piperidine-1-carboxylate (1055 mg, 2.62 mmol), 1,4-dioxane (8 mL), Cs₂CO₃ (2.32 g, 7.13 mmol) in water (2 mL) and Pd(dppf)Cl₂ (262 mg, 0.357 mmol) afforded the title compound (1.313 g, 95%) as a beige powder. LCMS: [M+H]⁺=551.55.

Step 2: 6-(2-amino-6-fluoro-5-(4-(piperidin-4-yloxy)phenyl)pyridin-3-yl)-7-fluoro-3,4-dihydroisoquinolin-1(2H)-one

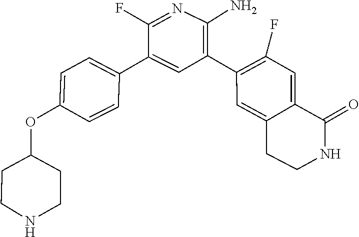 [SMILES: Nc1nc(F)c(-c2ccc(OC3CCNCC3)cc2)cc1-c1cc2c(cc1F)C(=O)NCC2]

A procedure similar to Example 144, Step 3 using tert-butyl 4-(4-(6-amino-2-fluoro-5-(7-fluoro-1-oxo-1,2,3,4-tetrahydroisoquinolin-6-yl)pyridin-3-yl)phenoxy)piperidine-1-carboxylate (1.3129 g, 2.384 mmol) and CH₂Cl₂ (4 mL) and TFA (2 mL, 26.1 mmol) afforded the title compound (1.094 g, 97%) as a beige powder which was used for the next step. LCMS: [M+H]⁺=451.48.

Step 3: 6-(2-amino-5-(4-((1-(2-cyclopropylacetyl)piperidin-4-yl)oxy)phenyl)-6-fluoropyridin-3-yl)-7-fluoro-3,4-dihydroisoquinolin-1(2H)-one

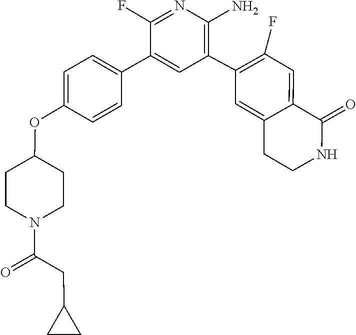 [SMILES: Nc1nc(F)c(-c2ccc(OC3CCN(C(=O)CC4CC4)CC3)cc2)cc1-c1cc2c(cc1F)C(=O)NCC2]

A procedure analogous to Example 191 using 6-(2-amino-6-fluoro-5-(4-(piperidin-4-yloxy)phenyl)pyridin-3-yl)-7-fluoro-3,4-dihydroisoquinolin-1(2H)-one (24.0 mg, 0.053 mmol) in CH₂Cl₂ (1 mL) was added N,N-diisopropylethylamine (0.046 mL, 0.266 mmol) and 2-cyclopropylacetyl chloride (7.58 mg, 0.064 mmol) afforded the title compound (13.9 mg, 47%) as a white powder. ¹H NMR (500 MHz, DMSO-d₆) δ 8.11 (br s, 1H), 7.60 (dd, J=10.1, 2.8 Hz, 2H), 7.48-7.39 (m, 3H), 7.02 (d, J=8.8 Hz, 2H), 6.24 (s, 2H), 4.70-4.59 (m, 1H), 3.92-3.80 (m, 1H), 3.72-3.64 (m, 1H), 3.43-3.37 (m, 2H), 3.28-3.20 (m, 1H), 2.96-2.87 (m, 2H), 2.27 (d, J=6.6 Hz, 2H), 2.00-1.85 (m, 2H), 1.64-1.55 (m, 1H), 1.54-1.45 (m, 1H), 1.01-0.89 (m, 1H), 0.49-0.40 (m, 2H), 0.15-0.08 (m, 2H); LCMS: [M+H]⁺=533.49.

### Example 324: 6-(2-amino-5-(4-((1-(cyclopropylmethyl)piperidin-4-yl)oxy)phenyl)-6-fluoropyridin-3-yl)-7-fluoro-3,4-dihydroisoquinolin-1(2H)-one (I-324)

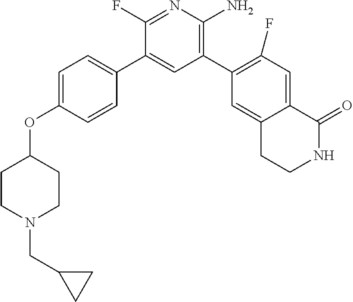 [SMILES: Nc1nc(F)c(-c2ccc(OC3CCN(CC4CC4)CC3)cc2)cc1-c1cc2c(cc1F)C(=O)NCC2]

A procedure similar to Example 186 using 6-(2-amino-6-fluoro-5-(4-(piperidin-4-yloxy)phenyl)pyridin-3-yl)-7-fluoro-3,4-dihydroisoquinolin-1(2H)-one (24.5 mg, 0.054 mmol) and (bromomethyl)cyclopropane (14.68 mg, 0.109 mmol) afforded the title compound (13.5 mg, 47%) as a white powder. ¹H NMR (500 MHz, DMSO-d₆) δ 8.10 (br s, 1H), 7.60 (dd, J=10.1, 7.5 Hz, 2H), 7.42 (d, J=7.5 Hz, 3H), 6.98 (d, J=8.7 Hz, 2H), 6.23 (s, 2H), 4.42-4.34 (m, 1H), 3.43-3.37 (m, 2H), 2.95-2.88 (m, 2H), 2.85-2.73 (m, 2H), 2.31-2.23 (m, 2H), 2.19 (br d, J=6.4 Hz, 2H), 2.00-1.90 (m, 2H), 1.68-1.57 (m, 2H), 0.88-0.78 (m, 1H), 0.51-0.41 (m, 2H), 0.07 (q, J=5.1 Hz, 2H); LCMS: [M+H]⁺=505.28.

### Example 325: 6-(2-amino-6-fluoro-5-(4-((1-(oxetan-3-yl)piperidin-4-yl)oxy)phenyl)pyridin-3-yl)-7-fluoro-3,4-dihydroisoquinolin-1(2H)-one (I-325)

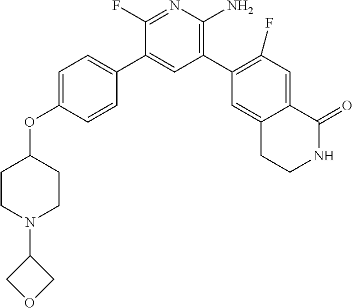 [SMILES: Nc1nc(F)c(-c2ccc(OC3CCN(C4COC4)CC3)cc2)cc1-c1cc2c(cc1F)C(=O)NCC2]

A procedure similar to Example 218 using 6-(2-amino-6-fluoro-5-(4-(piperidin-4-yloxy)phenyl)pyridin-3-yl)-7-fluoro-3,4-dihydroisoquinolin-1(2H)-one (26.1 mg, 0.058 mmol) in CH₂Cl₂ (1 ml) and oxetan-3-one (45 mg, 0.624 mmol) followed by 2 drops of glacial AcOH (40 mg, 0.666 mmol) afforded the title compound (10.0 mg, 32%) as a white powder. ¹H NMR (500 MHz, DMSO-d₆) δ 8.11 (br s, 1H), 7.60 (dd, J=10.1, 6.4 Hz, 2H), 7.46-7.40 (m, 3H), 6.99 (d, J=8.8 Hz, 2H), 6.23 (s, 2H), 4.53 (t, J=6.5 Hz, 2H), 4.42 (t, J=6.1 Hz, 3H), 3.40 (br s, 4H), 3.28-3.20 (m, 1H), 2.91 (s, 2H), 2.14-2.05 (m, 2H), 1.99-1.91 (m, 2H), 1.69-1.59 (m, 2H); LCMS: [M+H]⁺=507.38.

### Example 326: 6-(5-(4-((1-acetylazetidin-3-yl)oxy)phenyl)-2-amino-6-fluoropyridin-3-yl)-3,4-dihydroisoquinolin-1(2H)-one (I-326)

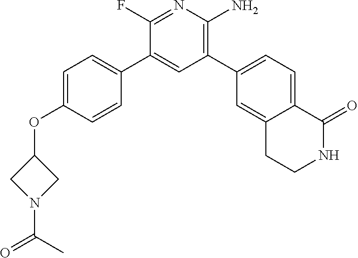 [SMILES: CC(=O)N1CC(Oc2ccc(-c3cc(-c4ccc5c(c4)CCNC5=O)c(N)nc3F)cc2)C1]

A procedure similar to Example 191 using 6-(2-amino-5-(4-(azetidin-3-yloxy)phenyl)-6-fluoropyridin-3-yl)-3,4-dihydroisoquinolin-1(2H)-one (24.2 mg, 0.060 mmol) and N,N-diisopropylethylamine (0.052 mL, 0.299 mmol) in CH₂Cl₂ (3 mL) and acetic anhydride (5.66 μl, 0.060 mmol) afforded the title compound (19.5 mg, 69%) as a white powder. ¹H NMR (500 MHz, DMSO-d₆) δ 7.94 (br s, 1H), 7.90 (d, J=7.9 Hz, 1H), 7.60 (d, J=10.1 Hz, 1H), 7.52-7.43 (m, 4H), 6.90 (d, J=8.8 Hz, 2H), 6.26 (s, 2H), 5.08-5.00 (m, 1H), 4.56 (dd, J=8.6, 6.6 Hz, 1H), 4.29 (dd, J=6.5, 10.5 Hz, 1H), 4.09 (dd, J=9.2, 3.7 Hz, 1H), 3.77 (dd, J=10.5, 3.7 Hz, 1H), 3.44-3.37 (m, 2H), 2.95 (s, 2H), 1.79 (s, 3H); LCMS: [M+H]⁺=447.42.

### Example 327: 6-(5-(4-((1-acetylpiperidin-4-yl)oxy)phenyl)-2-amino-6-fluoropyridin-3-yl)-7-fluoro-3,4-dihydroisoquinolin-1(2H)-one (I-327)

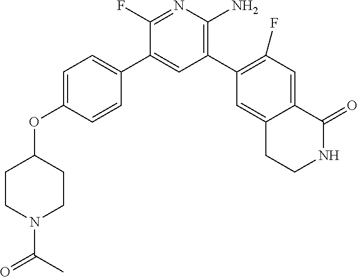 [SMILES: CC(=O)N1CCC(Oc2ccc(-c3cc(-c4cc5c(cc4F)C(=O)NCC5)c(N)nc3F)cc2)CC1]

A procedure similar to Example 191 using 6-(2-amino-6-fluoro-5-(4-(piperidin-4-yloxy)phenyl)pyridin-3-yl)-7-fluoro-3,4-dihydroisoquinolin-1(2H)-one (24.9 mg, 0.055 mmol), N,N-diisopropylethylamine (0.048 ml, 0.276 mmol) in CH₂Cl₂ (3 mL) and acetic anhydride (5.22 μl, 0.055 mmol) afforded the title compound (20.1 mg, 70%) as a beige powder. ¹H NMR (500 MHz, DMSO-d₆) δ 8.11 (br s, 1H), 7.60 (dd, J=2.6, 10.1 Hz, 2H), 7.48-7.40 (m, 3H), 7.02 (d, J=8.9 Hz, 2H), 6.24 (s, 2H), 4.68-4.60 (m, 1H), 3.88-3.79 (m, 1H), 3.70-3.62 (m, 1H), 3.43-3.38 (m, 2H), 3.37-3.34 (m, 1H), 3.28-3.20 (m, 1H), 2.96-2.88 (m, 2H), 2.01 (s, 3H), 1.99-1.93 (m, 1H), 1.93-1.85 (m, 1H), 1.66-1.57 (m, 1H), 1.55-1.45 (m, 1H); LCMS: [M+H]⁺=493.46.

### Example 328: 6-(2-amino-6-fluoro-5-(2-isopropyl-1,2,3,4-tetrahydroisoquinolin-6-yl)pyridin-3-yl)-3,4-dihydroisoquinolin-1(2H)-one (I-328)

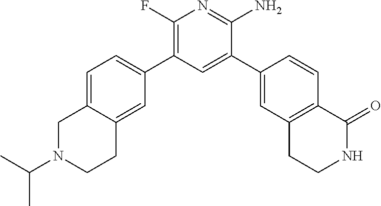 [SMILES: CC(C)N1CCc2cc(-c3cc(-c4ccc5c(c4)CCNC5=O)c(N)nc3F)ccc2C1]

To a solution of 6-(2-amino-6-fluoro-5-(1,2,3,4-tetrahydroisoquinolin-6-yl)pyridin-3-yl)-3,4-dihydroisoquinolin-1(2H)-one, trifluoroacetate (25 mg, 0.050 mmol) and N,N-diisopropylethylamine (0.087 ml, 0.498 mmol) in DMF (1.0 mL) was added 2-iodopropane (42.3 mg, 0.249 mmol). The reaction was stirred at RT for 72 h, concentrated onto Celite® and purified by silica gel chromatography eluting with 0-10% MeOH/DCM+1% NH₄OH, followed by reverse phase chromatography (C18, 0-100% ACN/H₂O) to afford the title compound (8.9 mg, 42%) as a beige solid. ¹H NMR (500 MHz, DMSO-d₆) δ 7.87 (br. s., 1H), 7.84 (d, J=8.0 Hz, 1H), 7.54 (d, J=10.0 Hz, 1H), 7.40 (d, J=Hz, 1H), 7.37 (s, 1H), 7.18-7.24 (m, 2H), 7.03 (d, J=8.0 Hz, 1H), 6.19 (s, 2H), 3.57 (s, 2H), 3.34 (td, J=6.5, 2.6 Hz, 2H), 2.89 (t, J=6.5 Hz, 2H), 2.76-2.82 (m, 1H), 2.71-2.76 (m, 2H), 2.60-2.65 (m, 2H), 0.99 (d, J=6.5 Hz, 6H); LCMS: [M+H]⁺=431.56.

### Example 329: 6-(2-amino-6-fluoro-5-(3-((4-(2-methoxyethyl)piperazin-1-yl)methyl)phenyl)pyridin-3-yl)-3,4-dihydroisoquinolin-1(2H)-one (I-329)

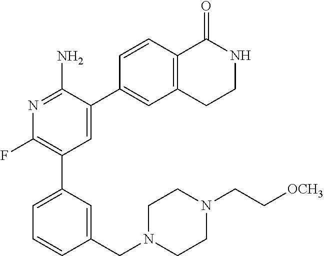 [SMILES: COCCN1CCN(Cc2cccc(-c3cc(-c4ccc5c(c4)CCNC5=O)c(N)nc3F)c2)CC1]

A procedure similar to Example 191 using 6-(2-amino-6-fluoro-5-(3-(piperazin-1-ylmethyl)phenyl)pyridin-3-yl)-3,4-dihydroisoquinolin-1(2H)-one, trifluoroacetate (25 mg, 0.046 mmol, preparation described in Example 265 Step 2) and N,N-diisopropylethylamine (0.080 mL, 0.458 mmol) in DMf (1.0 mL) was added 2-bromoethyl methyl ether (31.8 mg, 0.229 mmol). The reaction was stirred at RT overnight. The reaction was concentrated onto Celite® and purified by silica gel chromatography eluting with 0-10% MeOH/DCM+1% NH₄OH, followed by reverse phase chromatography (C18, 0-100% ACN/H₂O) to afford the title compound (10.3 mg, 46%) as a white solid. ¹H NMR (500 MHz, DMSO-d₆) δ 7.95 (br. s., 1H), 7.91 (d, J=8.0 Hz, 1H), 7.64 (d, J=10.0 Hz, 1H), 7.41-7.51 (m, 4H), 7.37 (t, J=7.5 Hz, 1H), 7.24 (d, J=7.5 Hz, 1H), 6.31 (s, 2H), 3.48 (s, 2H), 3.38-3.44 (m, 4H), 3.21 (s, 3H), 2.96 (t, J=6.4 Hz, 2H), 2.44 (t, J=5.9 Hz, 2H), 2.39 (br. s., 5H); LCMS: [M+H]⁺=490.67.

### Example 330: 6-(2-amino-6-fluoro-5-(3-((4-isopropylpiperazin-1-yl)methyl)phenyl)pyridin-3-yl)-3,4-dihydroisoquinolin-1(2H)-one (I-330)

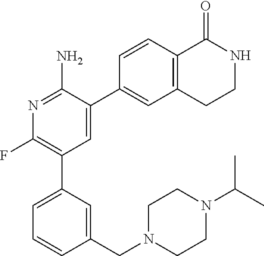 [SMILES: CC(C)N1CCN(Cc2cccc(-c3cc(-c4ccc5c(c4)CCNC5=O)c(N)nc3F)c2)CC1]

A procedure similar to Example 306, Step 3 using 6-(2-amino-6-fluoro-5-(3-(piperazin-1-ylmethyl)phenyl)pyridin-3-yl)-3,4-dihydroisoquinolin-1(2H)-one, trifluoroacetate (25 mg, 0.046 mmol) and N,N-diisopropylethylamine (0.080 mL, 0.458 mmol) in DMF (1.0 mL) and 2-iodopropane (39.0 mg, 0.229 mmol) afforded the title compound (3.2 mg, 15%) as a white solid. ¹H NMR (500 MHz, DMSO-d₆) δ 7.87 (br. s., 1H), 7.84 (d, J=7.8 Hz, 1H), 7.56 (d, J=10.2 Hz, 1H), 7.33-7.43 (m, 4H), 7.29 (t, J=7.6 Hz, 1H), 7.16 (d, J=7.5 Hz, 1H), 6.24 (s, 2H), 3.40 (s, 2H), 3.34 (td, J=6.4, 2.6 Hz, 2H), 2.88 (t, J=6.4 Hz, 2H), 2.48-2.54 (m, 1H), 2.25-2.39 (m, 7H), 0.87 (d, J=6.5 Hz, 6H); LCMS: [M+H]⁺=474.50.

### Example 331: 6-(2-amino-6-fluoro-5-(3-(4-methylpiperazine-1-carbonyl)phenyl)pyridin-3-yl)-3,4-dihydroisoquinolin-1(2H)-one (I-331)

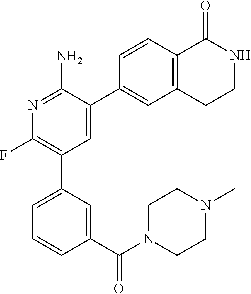 [SMILES: CN1CCN(C(=O)c2cccc(-c3cc(-c4ccc5c(c4)CCNC5=O)c(N)nc3F)c2)CC1]

A procedure analogous to Example 246 with 6-(2-amino-5-bromo-6-fluoropyridin-3-yl)-3,4-dihydroisoquinolin-1(2H)-one (50 mg, 0.149 mmol), 3-(4-methylpiperazine-1-carbonyl)phenylboronic acid, pinacol ester (58.9 mg, 0.178 mmol), Cs₂CO₃ (121 mg, 0.372 mmol), DME (4 mL), water (1.3 mL) and PdCl₂dppf (10.88 mg, 0.015 mmol) afforded the title compound (43.4 mg, 64%) as a white solid. ¹H NMR (500 MHz, DMSO-d₆) δ 7.95 (br. s., 1H), 7.95 (br. s., 1H), 7.92 (d, J=8.0 Hz, 1H), 7.69 (d, J=10.2 Hz, 1H), 7.55 (s, 1H), 7.45-7.52 (m, 3H), 7.31 (d, J=7.6 Hz, 1H), 6.39 (s, 2H), 3.62 (br. s., 2H), 3.41 (td, J=6.5, 2.6 Hz, 2H), 2.96 (t, J=6.5 Hz, 2H), 2.49-2.54 (m, 1H), 2.23-2.43 (m, 4 H), 2.19 (s, 3H); LCMS: [M+H]⁺=460.58.

### Example 332: 6-(2-amino-5-(3-amino-4-(4-methylpiperazin-1-yl)phenyl)-6-fluoropyridin-3-yl)-3,4-dihydroisoquinolin-1(2H)-one (I-332)

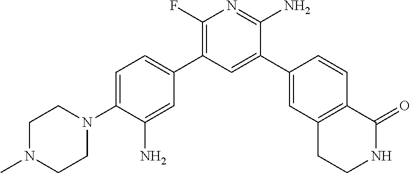 [SMILES: CN1CCN(c2ccc(-c3cc(-c4ccc5c(c4)CCNC5=O)c(N)nc3F)cc2N)CC1]

A procedure similar to Example 246 using 6-(2-amino-6-fluoro-5-(4,4,5,5-tetramethyl-1,3,2-dioxaborolan-2-yl)pyridin-3-yl)-3,4-dihydroisoquinolin-1(2H)-one (134 mg, 0.350 mmol), 5-bromo-2-(4-methylpiperazin-1-yl)aniline dihydrochloride (100 mg, 0.291 mmol), Cs₂CO₃ (237 mg, 0.729 mmol), DME (12 mL), water (4 mL) and PdCl₂dppf (21.33 mg, 0.029 mmol) the title compound (43.7 mg, 34%) as a white solid. ¹H NMR (500 MHz, DMSO-d₆) δ 7.94 (br. s., 1H), 7.91 (d, J=8.0 Hz, 1H), 7.53 (d, J=10.0 Hz, 1H), 7.44-7.48 (m, 1H), 7.43 (s, 1H), 6.92 (d, J=8.2 Hz, 1H), 6.88 (s, 1H), 6.74 (d, J=8.2 Hz, 1H), 6.19 (s, 2H), 4.74 (s, 2H), 3.41 (td, J=6.5, 2.6 Hz, 2H), 2.95 (t, J=6.5 Hz, 2H), 2.82 (br. s., 4H), 2.50-2.51 (m, 4H), 2.24 (s, 3H); LCMS: [M+H]⁺=447.61.

### Example 333: 6,6′-(2-amino-6-fluoropyridine-3,5-diyl)bis(3,4-dihydroisoquinolin-1(2H)-one) (I-333)

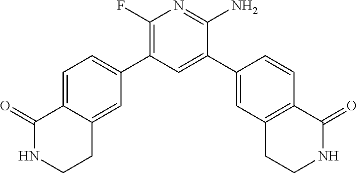 [SMILES: Nc1nc(F)c(-c2ccc3c(c2)CCNC3=O)cc1-c1ccc2c(c1)CCNC2=O]

A procedure analogous to Example 246 using 6-(2-amino-5-bromo-6-fluoropyridin-3-yl)-3,4-dihydroisoquinolin-1(2H)-one (50 mg, 0.149 mmol), 3,4-dihydro-1(2H)-isoquinolinone-6-boronic acid pinacol ester (48.8 mg, 0.178 mmol), Cs₂CO₃ (121 mg, 0.372 mmol), DME (4 mL), water (1.3 mL) and PdCl₂dppf (10.88 mg, 0.015 mmol) afforded the title compound (14.7 mg, 25%) as beige solid. ¹H NMR (500 MHz, DMSO-d₆) δ 7.88 (br. s., 1H), 7.82-7.86 (m, 2H), 7.79 (d, J=8.1 Hz, 1H), 7.65 (d, J=10.2 Hz, 1H), 7.47 (d, J=8.2 Hz, 1H), 7.45 (s, 1H), 7.41 (d, J=8.0 Hz, 1H), 7.39 (s, 1H), 6.36 (s, 2H), 3.30-3.37 (m, 4H), 2.84-2.92 (m, 4H); LCMS: [M+H]⁺=403.48.

### Example 334: 6-(2-amino-6-fluoro-5-(1,2,3,4-tetrahydroisoquinolin-7-yl)pyridin-3-yl)-3,4-dihydroisoquinolin-1(2H)-one 2,2,2-trifluoroacetate (I-334)

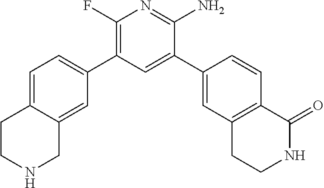 [SMILES: Nc1nc(F)c(-c2ccc3c(c2)CNCC3)cc1-c1ccc2c(c1)CCNC2=O]

Step 1: tert-butyl 7-(6-amino-2-fluoro-5-(1-oxo-1,2,3,4-tetrahydroisoquinolin-6-yl)pyridin-3-yl)-3,4-dihydroisoquinoline-2(1H)-carboxylate

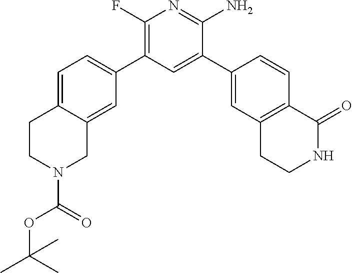 [SMILES: CC(C)(C)OC(=O)N1CCc2ccc(-c3cc(-c4ccc5c(c4)CCNC5=O)c(N)nc3F)cc2C1]

To a suspension of 6-(2-amino-5-bromo-6-fluoropyridin-3-yl)-3,4-dihydroisoquinolin-1(2H)-one (110 mg, 0.327 mmol), tert-butyl 7-(4,4,5,5-tetramethyl-1,3,2-dioxaborolan-2-yl)-3,4-dihydroisoquinoline-2(1H)-carboxylate (141 mg, 0.393 mmol) and Cs₂CO₃ (267 mg, 0.818 mmol) in DME (4 mL) and water (1.3 mL) was added PdCl₂dppf (23.94 mg, 0.033 mmol). The mixture was flushed with nitrogen and heated in the microwave at 90° C. for 2 h. The reaction was concentrated onto Celite® and purified by silica gel chromatography eluting with 0-10% MeOH/DCM+1% NH₄OH to afford the product (141 mg, 88%) as a beige solid. LCMS: [M+H]⁺=489.48.

Step 2: 6-(2-amino-6-fluoro-5-(1,2,3,4-tetrahydroisoquinolin-7-yl)pyridin-3-yl)-3,4-dihydroisoquinolin-1(2H)-one

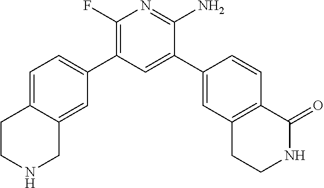 [SMILES: Nc1nc(F)c(-c2ccc3c(c2)CNCC3)cc1-c1ccc2c(c1)CCNC2=O]

To a solution of tert-butyl 7-(6-amino-2-fluoro-5-(1-oxo-1,2,3,4-tetrahydroisoquinolin-6-yl)pyridin-3-yl)-3,4-dihydroisoquinoline-2(1H)-carboxylate (141 mg, 0.289 mmol) in CH₂Cl₂ (5.0 mL) was added TFA (0.66 mL, 8.66 mmol). The reaction was stirred at RT overnight. The mixture was concentrated onto Celite® and purified by reverse phase chromatography (C18, 0-100% MeCN—H₂O) to give the trifluoroacetate salt of the title compound (132 mg, 91%) as a pale yellow solid. ¹H NMR (500 MHz, DMSO-d₆) δ 8.95 (br. s., 2H), 7.96 (br. s., 1H), 7.92 (d, J=7.8 Hz, 1H), 7.64 (d, J=10.2 Hz, 1H), 7.45-7.50 (m, 2H), 7.44 (s, 2H), 7.29 (d, J=8.2 Hz, 1H), 6.36 (s, 2H), 4.31 (s, 2H), 3.42 (t, J=6.4 Hz, 4H), 3.01 (t, J=6.2 Hz, 2H), 2.96 (t, J=6.5 Hz, 2H); LCMS: [M+H]⁺=389.50.

### Example 335: 6-(2-amino-6-fluoro-5-(2-isopropyl-1,2,3,4-tetrahydroisoquinolin-7-yl)pyridin-3-yl)-3,4-dihydroisoquinolin-1(2H)-one (I-335)

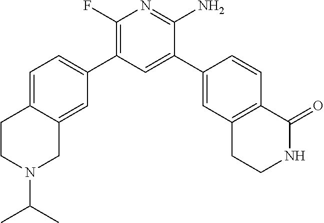 [SMILES: CC(C)N1CCc2ccc(-c3cc(-c4ccc5c(c4)CCNC5=O)c(N)nc3F)cc2C1]

A procedure similar to Example 306, Step 3 using 6-(2-amino-6-fluoro-5-(1,2,3,4-tetrahydroisoquinolin-7-yl)pyridin-3-yl)-3,4-dihydroisoquinolin-1(2H)-one-TFA (25 mg, 0.050 mmol), N,N-diisopropylethylamine (0.087 mL, 0.498 mmol), DMF (1.0 mL) and 2-iodopropane (42.3 mg, 0.249 mmol) afforded the title compound (4.8 mg, 2%) as a white solid. ¹H NMR (500 MHz, DMSO-d₆) δ 7.87 (br. s., 1H), 7.84 (d, J=8.0 Hz, 1H), 7.55 (d, J=10.2 Hz, 1H), 7.39 (d, J=8.1 Hz, 1H), 7.37 (s, 1H), 7.22 (d, J=8.0 Hz, 1H), 7.18 (s, 1H), 7.05 (d, J=8.0 Hz, 1H), 6.19 (s, 2H), 3.59 (s, 2H), 3.34 (td, J=6.42, 2.6 Hz, 2H), 2.88 (t, J=6.5 Hz, 2H), 2.78 (dt, J=13.1, 6.5 Hz, 1H), 2.69-2.74 (m, 2H), 2.60-2.65 (m, 2H), 0.99 (d, J=6.5 Hz, 6H); LCMS: [M+H]⁺=431.56.

### Example 336: 6-(2-amino-6-fluoro-5-(4-(4-isopropylpiperazin-1-yl)phenyl)pyridin-3-yl)-8-fluoro-3,4-dihydroisoquinolin-1(2H)-one (I-336)

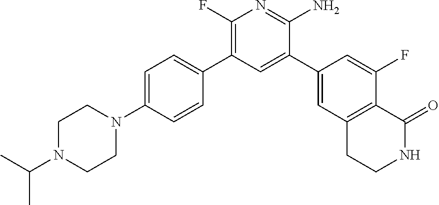 [SMILES: CC(C)N1CCN(c2ccc(-c3cc(-c4cc(F)c5c(c4)CCNC5=O)c(N)nc3F)cc2)CC1]

A procedure analogous to Example 246 using 6-(2-amino-5-bromo-6-fluoropyridin-3-yl)-8-fluoro-3,4-dihydroisoquinolin-1(2H)-one (41 mg, 0.116 mmol), 4-(4-isopropylpiperazinyl)phenylboronic acid, pinacol ester (45.9 mg, 0.139 mmol) Cs₂CO₃ (94 mg, 0.289 mmol), DME (4 mL), water (1.3 mL) and PdCl₂dppf (8.47 mg, 0.012 mmol) afforded the title compound (34.7 mg, 63%) as a white solid. ¹H NMR (500 MHz, DMSO-d₆) δ 7.99 (br. s., 1H), 7.62 (d, J=10.2 Hz, 1H), 7.41 (d, J=8.0 Hz, 2H), 7.26 (d, J=12.0 Hz, 1H) 7.30 (s, 1H), 6.97 (d, J=8.8 Hz, 2H), 6.29 (s, 2H), 3.12-3.19 (m, 4H), 2.94 (t, J=6.2 Hz, 2H), 2.68 (dt, J=13.0, 6.5 Hz, 1H), 2.55-2.61 (m, 4H), 1.01 (d, J=6.5 Hz, 6H); LCMS: [M+H]+=478.51.

### Example 337: 6-(2-amino-5-(4-((1-(2-cyclopropylacetyl)piperidin-4-yl)oxy)phenyl)-6-fluoropyridin-3-yl)-3,4-dihydroisoquinolin-1(2H)-one (I-337)

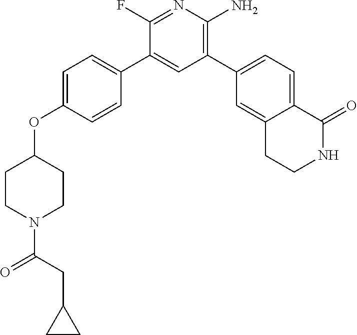 [SMILES: Nc1nc(F)c(-c2ccc(OC3CCN(C(=O)CC4CC4)CC3)cc2)cc1-c1ccc2c(c1)CCNC2=O]

A procedure similar to Example 191 using 6-(2-amino-6-fluoro-5-(4-(piperidin-4-yloxy)phenyl)pyridin-3-yl)-3,4-dihydroisoquinolin-1(2H)-one (28.1 mg, 0.065 mmol) in CH₂Cl₂ (1 mL), N,N-diisopropylethylamine (0.057 ml, 0.325 mmol) and 2-cyclopropylacetyl chloride (9.24 mg, 0.078 mmol) afforded (20.1 mg, 57%) as a white powder. ¹H NMR (500 MHz, DMSO-d₆) δ 7.94 (br s, 1H), 7.90 (d, J=7.9 Hz, 1H), 7.60 (d, J=10.1 Hz, 1H), 7.50-7.43 (m, 4H), 7.03 (d, J=8.8 Hz, 2H), 6.23 (s, 2H), 4.68-4.61 (m, 1H), 3.92-3.82 (m, 1H), 3.73-3.64 (m, 1H), 3.40 (dt, J=2.6, 6.4 Hz, 2H), 3.29-3.21 (m, 1H), 2.95 (br t, J=6.5 Hz, 2H), 2.27 (d, J=6.6 Hz, 2H), 2.01-1.86 (m, 2H), 1.65-1.56 (m, 1H), 1.55-1.45 (m, 1H), 0.99-0.91 (m, 1H), 0.50-0.40 (m, 2H), 0.16-0.08 (m, 2H); LCMS: [M+H]⁺=515.51.

### Example 338: 6-(2-amino-6-fluoro-5-(4-((1-(oxetan-3-yl)piperidin-4-yl)oxy)phenyl)pyridin-3-yl)-3,4-dihydroisoquinolin-1(2H)-one (I-338)

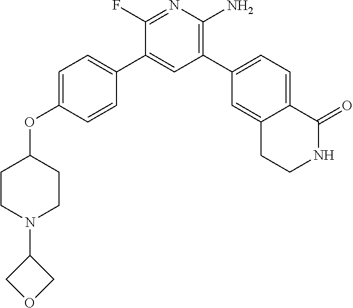 [SMILES: Nc1nc(F)c(-c2ccc(OC3CCN(C4COC4)CC3)cc2)cc1-c1ccc2c(c1)CCNC2=O]

A procedure similar to Example 218 using 6-(2-amino-6-fluoro-5-(4-(piperidin-4-yloxy)phenyl)pyridin-3-yl)-3,4-dihydroisoquinolin-1(2H)-one (25.4 mg, 0.059 mmol), oxetan-3-one (45 mg, 0.624 mmol) in CH₂Cl₂ (1 mL), 2 drops of glacial AcOH (40 mg, 0.666 mmol) and NaBH(OAc)₃ (137 mg, 0.646 mmol) afforded the title compound (3.3 mg, 11%) as a white powder. ¹H NMR (500 MHz, DMSO-d₆) δ 7.94 (br s, 1H), 7.90 (br d, J=7.5 Hz, 1H), 7.59 (br d, J=9.7 Hz, 1H), 7.51-7.38 (m, 3H), 6.99 (br d, J=7.0 Hz, 2H), 6.23 (br s, 2H), 5.76 (br s, 1H), 4.75-4.67 (m, 1H), 4.67-4.60 (m, 1H), 4.57-4.50 (m, 2H), 4.48-4.37 (m, 2H), 3.45-3.32 (m, 4H), 3.01-2.88 (m, 2H), 2.15-2.05 (m, 2H), 2.01-1.88 (m, 2H), 1.71-1.57 (m, 2H); LCMS: [M+H]⁺=489.48.

### Example 339: 6-(2-amino-5-(4-(azetidin-3-yloxy)phenyl)-6-fluoropyridin-3-yl)-7-fluoro-3,4-dihydroisoquinolin-1(2H)-one 2,2,2-trifluoroacetate (I-339)

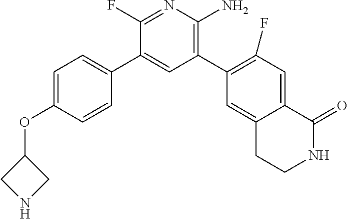 [SMILES: Nc1nc(F)c(-c2ccc(OC3CNC3)cc2)cc1-c1cc2c(cc1F)C(=O)NCC2]

Step 1: tert-butyl 3-(4-(6-amino-2-fluoro-5-(7-fluoro-1-oxo-1,2,3,4-tetrahydroisoquinolin-6-yl)pyridin-3-yl)phenoxy)azetidine-1-carboxylate

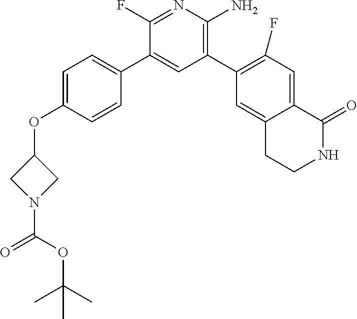 [SMILES: CC(C)(C)OC(=O)N1CC(Oc2ccc(-c3cc(-c4cc5c(cc4F)C(=O)NCC5)c(N)nc3F)cc2)C1]

In a vial were added 6-(2-amino-5-bromo-6-fluoropyridin-3-yl)-7-fluoro-3,4-dihydroisoquinolin-1(2H)-one (781 mg, 2.205 mmol), tert-butyl 3-(4-(4,4,5,5-tetramethyl-1,3,2-dioxaborolan-2-yl)phenoxy)azetidine-1-carboxylate (910.3 mg, 2.43 mmol) and 1,4-dioxane (8 ml). Then a solution of Cs₂CO₃ (2155 mg, 6.62 mmol) in Water (2 mL) was added at RT and the reaction mixture was degassed under argon for 15 minutes. Then [1,12-bis(diphenylphosphino)ferrocene]dichloropalladium(II) (242 mg, 0.331 mmol) was added and the reaction mixture was heated at 90° C. for overnight. The reaction mixture was dry loaded onto Celite® and purified by flash chromatography (0-10% DCM/MeOH) to afford the product (1.152 g, 100%) as a beige powder. [M+H]⁺ 523.48.

Step 2: 6-(2-amino-5-(4-(azetidin-3-yloxy)phenyl)-6-fluoropyridin-3-yl)-7-fluoro-3,4-dihydroisoquinolin-1(2H)-one

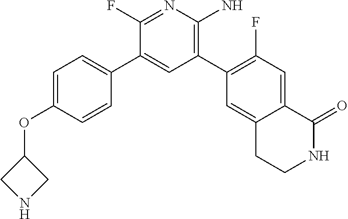 [SMILES: Nc1nc(F)c(-c2ccc(OC3CNC3)cc2)cc1-c1cc2c(cc1F)C(=O)NCC2]

A procedure similar to Example 144, Step 3 with tert-butyl 3-(4-(6-amino-2-fluoro-5-(7-fluoro-1-oxo-1,2,3,4-tetrahydroisoquinolin-6-yl)pyridin-3-yl)phenoxy)azetidine-1-carboxylate (1152 mg, 2.21 mmol) and TFA (4 mL, 52.2 mmol) in CH₂Cl₂ (10 mL) afforded the title compound TFA salt (897.7 mg, 72%) as a beige powder. ¹H NMR (500 MHz, DMSO-d₆) δ 8.90 (br s, 2H from TFA), 8.11 (br s, 1H), 7.61 (dd, J=10.1, 3.3 Hz, 2H), 7.49 (d, J=8.1 Hz, 2H), 7.42 (d, J=7.0 Hz, 1H), 6.91 (d, J=8.8 Hz, 2H), 6.28 (s, 2H), 5.14-5.06 (m, 1H), 4.50-4.42 (m, 2H), 4.03-3.98 (m, 2H), 3.43-3.38 (m, 2H), 2.95-2.88 (m, 2H); LCMS: [M+H]⁺=423.34.

### Example 340: 6-(2-amino-6-fluoro-5-(4-(3-fluoroazetidin-3-yl)phenyl)pyridin-3-yl)-3,4-dihydroisoquinolin-1(2H)-one 2,2,2-trifluoroacetate (I-340)

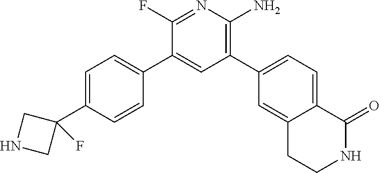 [SMILES: Nc1nc(F)c(-c2ccc(C3(F)CNC3)cc2)cc1-c1ccc2c(c1)CCNC2=O]

Step 1: tert-butyl 3-(4-(6-amino-2-fluoro-5-(1-oxo-1,2,3,4-tetrahydroisoquinolin-6-yl)pyridin-3-yl)phenyl)-3-fluoroazetidine-1-carboxylate

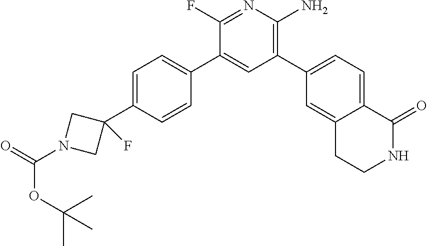 [SMILES: CC(C)(C)OC(=O)N1CC(F)(c2ccc(-c3cc(-c4ccc5c(c4)CCNC5=O)c(N)nc3F)cc2)C1]

A procedure similar to Example 43, Step 5 using tert-butyl 3-(4-bromophenyl)-3-fluoroazetidine-1-carboxylate (516 mg, 1.56 mmol), 6-(2-amino-6-fluoro-5-(4,4,5,5-tetramethyl-1,3,2-dioxaborolan-2-yl)pyridin-3-yl)-3,4-dihydroisoquinolin-1(2H)-one (719 mg, 1.88 mmol), 1,4-dioxane (10 mL) and K₃PO₄ (995 mg, 4.69 mmol) in water (2 mL) and XPhos Pd G2 (123 mg, 0.156 mmol) afforded the product (792 mg, 100%) as a beige powder. LCMS: [M+H]⁺=507.53.

Step 2: 6-(2-amino-6-fluoro-5-(4-(3-fluoroazetidin-3-yl)phenyl)pyridin-3-yl)-3,4-dihydroisoquinolin-1(2H)-one

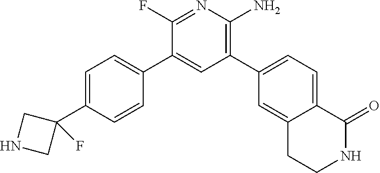 [SMILES: Nc1nc(F)c(-c2ccc(C3(F)CNC3)cc2)cc1-c1ccc2c(c1)CCNC2=O]

A procedure similar to Example 144, Step 3 using tert-butyl 3-(4-(6-amino-2-fluoro-5-(1-oxo-1,2,3,4-tetrahydroisoquinolin-6-yl)pyridin-3-yl)phenyl)-3-fluoroazetidine-1-carboxylate (0.792 g, 1.56 mmol), TFA (4 mL, 52.2 mmol) and CH₂Cl₂ (10 mL) afforded the title compound (700.6 mg, 82%) as a beige powder. ¹H NMR (500 MHz, DMSO-d₆) δ 9.26 (br s, 2H from TFA), 7.96 (br s, 1H), 7.92 (d, J=7.8 Hz, 1H), 7.73-7.67 (m, 3H), 7.61 (d, J=8.2 Hz, 2H), 7.47 (d, J=8.1 Hz, 1H), 7.45 (s, 1H), 6.42 (s, 2H), 4.67-4.57 (m, 2H), 4.55-4.45 (m, 2H), 3.41 (dt, J=6.4, 2.6 Hz, 2H), 2.95 (t, J=6.5 Hz, 2H); LCMS: [M+H]⁺=407.39.

### Example 341: 6-(5-(4-((1-acetylazetidin-3-yl)oxy)phenyl)-2-amino-6-fluoropyridin-3-yl)-7-fluoro-3,4-dihydroisoquinolin-1(2H)-one (I-341)

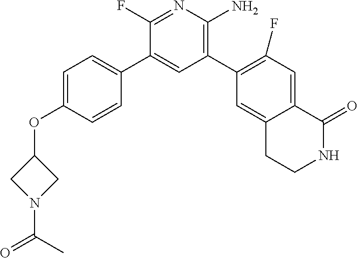 [SMILES: CC(=O)N1CC(Oc2ccc(-c3cc(-c4cc5c(cc4F)C(=O)NCC5)c(N)nc3F)cc2)C1]

A procedure similar to Example 191 using 6-(2-amino-5-(4-(azetidin-3-yloxy)phenyl)-6-fluoropyridin-3-yl)-7-fluoro-3,4-dihydroisoquinolin-1(2H)-one, trifluoroacetic acid (23.4 mg, 0.044 mmol) and N,N-diisopropylethylamine (0.038 mL, 0.218 mmol) in CH₂Cl₂ (3 mL) and acetic anhydride (4.12 μl, 0.044 mmol) afforded the title compound (15.6 mg, 73%) as a white powder. ¹H NMR (500 MHz, DMSO-d₆) δ 8.11 (br s, 1H), 7.61 (d, J=10.1 Hz, 2H), 7.47 (br d, J=8.1 Hz, 2H), 7.43 (d, J=7.0 Hz, 1H), 6.89 (d, J=8.7 Hz, 2H), 6.27 (s, 2H), 5.09-5.01 (m, 1H), 4.55 (dd, J=8.9, 6.8 Hz, 1H), 4.28 (dd, J=10.3, 6.5 Hz, 1H), 4.09 (dd, J=9.3, 3.4 Hz, 1H), 3.77 (dd, J=10.5, 3.6 Hz, 1H), 3.43-3.38 (m, 2H), 2.92 (br t, J=6.4 Hz, 2H), 1.79 (s, 3H); LCMS: [M+H]⁺=465.40.

### Example 342: 6-(5-(4-(1-acetyl-3-fluoroazetidin-3-yl)phenyl)-2-amino-6-fluoropyridin-3-yl)-3,4-dihydroisoquinolin-1(2H)-one (I-342)

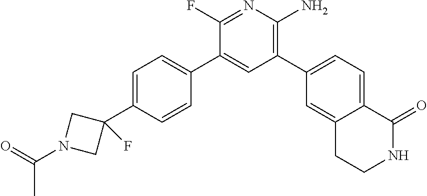 [SMILES: CC(=O)N1CC(F)(c2ccc(-c3cc(-c4ccc5c(c4)CCNC5=O)c(N)nc3F)cc2)C1]

A procedure similar to Example 191 with 6-(2-amino-6-fluoro-5-(4-(3-fluoroazetidin-3-yl)phenyl)pyridin-3-yl)-3,4-dihydroisoquinolin-1(2H)-one, trifluoroacetic acid (24.3 mg, 0.047 mmol), N,N-diisopropylethylamine (0.041 ml, 0.233 mmol) in CH₂Cl₂ (3 mL) and acetic anhydride (4.41 μl, 0.047 mmol) afforded the title compound (15.5 mg, 70%) as a white powder. ¹H NMR (500 MHz, DMSO-d₆) δ 7.92 (br dd, J=7.6, 12.0 Hz, 2H), 7.76-7.38 (m, 7H), 6.38 (br s, 2H), 4.72-4.52 (m, 2H), 4.39-4.20 (m, 2H), 3.45-3.38 (m, 2H), 3.02-2.89 (m, 2H), 1.87 (br s, 3H); LCMS: [M+H]⁺=449.37.

### Example 343: 6-(2-amino-6-fluoro-5-(4-((1R,5S)-3-methyl-3-azabicyclo[3.1.0]hexan-1-yl)phenyl)pyridin-3-yl)-7-fluoro-3,4-dihydroisoquinolin-1(2H)-one (I-343)

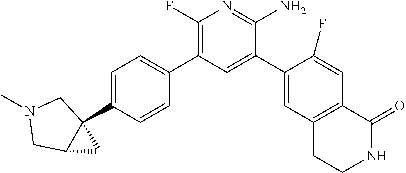 [SMILES: CN1C[C@H]2C[C@@]2(c2ccc(-c3cc(-c4cc5c(cc4F)C(=O)NCC5)c(N)nc3F)cc2)C1]

A procedure analogous to Example 246 using 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (40 mg, 0.100 mmol), (1R,5S)-1-(4-bromophenyl)-3-methyl-3-azabicyclo[3.1.0]hexane (30.2 mg, 0.120 mmol, preparation described in Example 14), Cs₂CO₃ (81 mg, 0.249 mmol), DME (4 mL), water (1.3 mL) and PdCl₂dppf (7.29 mg, 9.97 μmol) afforded the title compound (14.4 mg, 32%) as an off-white solid. ¹H NMR (500 MHz, DMSO-d₆) δ 8.11 (br s, 1H), 7.61 (dd, J=10.0, 5.0 Hz, 2H), 7.48-7.40 (m, 3H), 7.17 (br d, J=8.1 Hz, 2H), 6.29 (br s, 2H), 3.40 (br d, J=4.2 Hz, 2H), 3.01 (br s, 1H), 2.92 (br t, J=6.2 Hz, 2H), 2.47-2.38 (m, 1H), 2.32 (br s, 3H), 1.81 (br s, 1H), 1.35 (br s, 1H), 0.78 (br s, 1H); LCMS: [M+H]⁺=447.42.

### Example 344: 6-(2-amino-6-fluoro-5-(4-((1S,5R)-3-methyl-3-azabicyclo[3.1.0]hexan-1-yl)phenyl)pyridin-3-yl)-7-fluoro-3,4-dihydroisoquinolin-1(2H)-one (I-344)

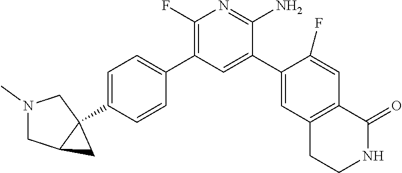 [SMILES: CN1C[C@@H]2C[C@]2(c2ccc(-c3cc(-c4cc5c(cc4F)C(=O)NCC5)c(N)nc3F)cc2)C1]

A procedure analogous to Example 246 using 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (40 mg, 0.100 mmol), (1 S,5R)-1-(4-bromophenyl)-3-methyl-3-azabicyclo[3.1.0]hexane (30.2 mg, 0.120 mmol, preparation described in Example 106), Cs₂CO₃ (81 mg, 0.249 mmol), DME (4 mL), water (1.3 mL) and PdCl₂dppf (7.29 mg, 9.97 μmol) afforded the title compound (13.7 mg, 31%) as an off-white solid. ¹H NMR (500 MHz, DMSO-d₆) δ 8.11 (br s, 1H), 7.62 (dd, J=5.4, 10.0 Hz, 2H), 7.49-7.41 (m, 3H), 7.19 (br d, J=7.9 Hz, 2H), 6.30 (br s, 2H), 3.40 (br d, J=4.0 Hz, 2H), 3.03 (br s, 1H), 2.92 (br t, J=6.2 Hz, 2H), 2.42-2.25 (m, 3H), 1.83 (br s, 1H), 1.36 (br t, J=3.9 Hz, 1H), 0.80 (br s, 1H); LCMS: [M+H]⁺=447.48.

### Example 345: 6-(2-amino-5-(2,3-difluoro-4-(4-isopropylpiperazin-1-yl)phenyl)-6-fluoropyridin-3-yl)-3,4-dihydroisoquinolin-1(2H)-one (I-345)

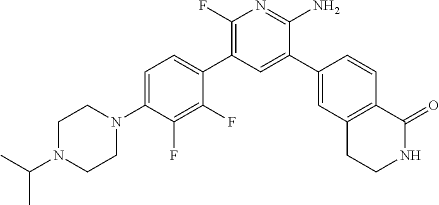 [SMILES: CC(C)N1CCN(c2ccc(-c3cc(-c4ccc5c(c4)CCNC5=O)c(N)nc3F)c(F)c2F)CC1]

Step 2: tert-butyl 4-(4-(6-amino-2-fluoro-5-(1-oxo-1,2,3,4-tetrahydroisoquinolin-6-yl)pyridin-3-yl)-2,3-difluorophenyl)piperazine-1-carboxylate

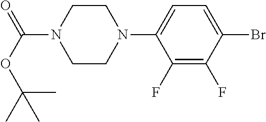 [SMILES: CC(C)(C)OC(=O)N1CCN(c2ccc(Br)c(F)c2F)CC1]

To a degassed suspension of 1,4-dibromo-2,3-difluorobenzene (0.5 g, 1.8 mmol), tert-butyl piperazine-1-carboxylate (0.37 g, 2.0 mmol), BINAP (0.079 g, 0.1 mmol), sodium tert-butoxide (0.21 g, 2.2 mmol) in toluene (10 mL) was added Pd₂(dba)₃ (0.08 g, 0.1 mmol) at RT under argon atmosphere. The reaction mixture was heated to 85° C. for 4 h before cooling to RT. The mixture was filtered through a Celite® bed, which was washed with EtOAc (300 mL). The combined filtrate was concentrated under reduced pressure to give a residue which was purified by column chromatography (silica gel, 100-200 mesh) eluting with 0-20% EtOAc in pet ether to afford the product (0.4 g, 58%) as an off white solid. LCMS: [M +H]⁺=377.31.

Step 2: tert-butyl 4-(4-(6-amino-2-fluoro-5-(1-oxo-1,2,3,4-tetrahydroisoquinolin-6-yl)pyridin-3-yl)-2,3-difluorophenyl)piperazine-1-carboxylate

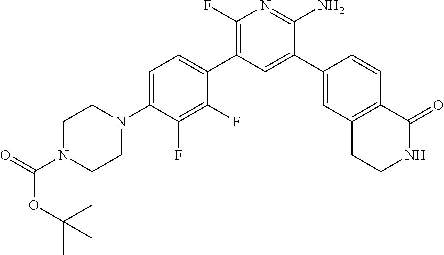 [SMILES: CC(C)(C)OC(=O)N1CCN(c2ccc(-c3cc(-c4ccc5c(c4)CCNC5=O)c(N)nc3F)c(F)c2F)CC1]

A procedure analogous to Example 246 using 6-(2-amino-6-fluoro-5-(4,4,5,5-tetramethyl-1,3,2-dioxaborolan-2-yl)pyridin-3-yl)-3,4-dihydroisoquinolin-1(2H)-one (98 mg, 0.254 mmol), tert-butyl 4-(4-bromo-2,3-difluorophenyl)piperazine-1-carboxylate (80 mg, 0.212 mmol), Cs₂CO₃ (173 mg, 0.530 mmol), DME (12 mL), water (4 mL) and PdCl₂dppf (15.52 mg, 0.021 mmol) afforded the product (30.4 mg, 26%) as a beige solid. LCMS: [M+H]+=554.66.

Step 2: 6-(2-amino-6-fluoro-5-(4-(piperidin-4-yl)phenyl)pyridin-3-yl)-7-fluoro-3,4-dihydroisoquinolin-1(2H)-one

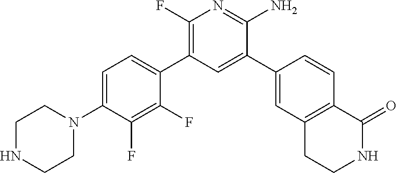 [SMILES: Nc1nc(F)c(-c2ccc(N3CCNCC3)c(F)c2F)cc1-c1ccc2c(c1)CCNC2=O]

To a solution of tert-butyl 4-(4-(6-amino-2-fluoro-5-(1-oxo-1,2,3,4-tetrahydroisoquinolin-6-yl)pyridin-3-yl)-2,3-difluorophenyl)piperazine-1-carboxylate (30 mg, 0.054 mmol) in CH₂Cl₂ (5.0 mL) was added TFA (0.12 mL, 1.63 mmol). The reaction was stirred at RT for 16 h and concentrated onto Celite® and purified by reverse phase chromatography (C18, 0-100% MeCN—H₂O) to give the TFA salt of the title compound (17 mg, 55%) as an off-white solid. LCMS: [M+H]⁺=454.50.

Step 3: 6-(2-amino-5-(2,3-difluoro-4-(4-isopropylpiperazin-1-yl)phenyl)-6-fluoropyridin-3-yl)-3,4-dihydroisoquinolin-1(2H)-one

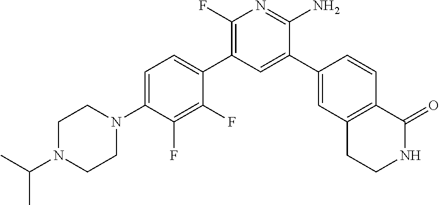 [SMILES: CC(C)N1CCN(c2ccc(-c3cc(-c4ccc5c(c4)CCNC5=O)c(N)nc3F)c(F)c2F)CC1]

A procedure similar to Example 306, Step 3 using 6-(2-amino-5-(2,3-difluoro-4-(piperazin-1-yl)phenyl)-6-fluoropyridin-3-yl)-3,4-dihydroisoquinolin-1(2H)-one, trifluoroacetate (17 mg, 0.030 mmol), N,N-diisopropylethylamine (0.052 mL, 0.300 mmol) in DMF (1.0 mL) and 2-iodopropane (25.5 mg, 0.150 mmol) afforded the title compound (2.4 mg, 16%) as an off-white solid. ¹H NMR (500 MHz, DMSO-d₆) δ 7.94 (br s, 1H), 7.90 (d, J=7.9 Hz, 1H), 7.52 (d, J=9.5 Hz, 1H), 7.47-7.41 (m, 2H), 7.18 (br t, J=7.9 Hz, 1H), 6.88 (br t, J=8.1 Hz, 1H), 6.41 (s, 2H), 3.41-3.38 (m, 2H), 3.14-3.01 (m, 4H), 2.94 (br t, J=6.5 Hz, 2H), 2.68 (br d, J=3.5 Hz, 1H), 2.66-2.56 (m, 4H), 1.01 (br d, J=4.2 Hz, 6H); LCMS: [M+H]+=496.43.

### Example 346: 6-(2-amino-5-(4-(1,1-dioxidothiomorpholino)phenyl)-6-fluoropyridin-3-yl)-7-fluoro-3,4-dihydroisoquinolin-1(2H)-one (I-346)

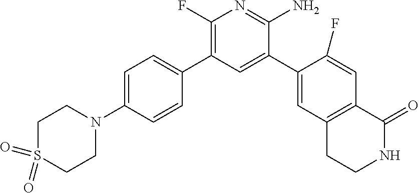 [SMILES: Nc1nc(F)c(-c2ccc(N3CCS(=O)(=O)CC3)cc2)cc1-c1cc2c(cc1F)C(=O)NCC2]

A procedure analogous to that of Example 177, Step 1 using 4-(4-bromophenyl)thiomorpholine-1,1-dione (50 mg, 0.172 mmol) with 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (97 mg, 0.241 mmol), [1,12-bis(diphenylphosphino)ferrocene]dichloropalladium(II) (17.65 mg, 0.024 mmol), Cs₂CO₃ (168 mg, 0.517 mmol), DME (3.5 mL) and water (1 mL) afforded the title compound as a beige solid (14 mg, 16%). ¹H NMR (500 MHz, DMSO-d₆) δ 8.18-8.06 (m, 1H), 7.67-7.58 (m, 2H), 7.51-7.39 (m, 3H), 7.22-6.99 (m, 2H), 6.32-6.13 (m, 2H), 3.87-3.78 (m, 4H), 3.43-3.41 (m, 2H), 3.13 (br s, 4H), 2.99-2.87 (m, 2H); LCMS: [M+H]⁺=485.42.

### Example 347: 6-(2-amino-6-fluoro-5-(4-thiomorpholinophenyl)pyridin-3-yl)-7-fluoro-3,4-dihydroisoquinolin-1(2H)-one (I-347)

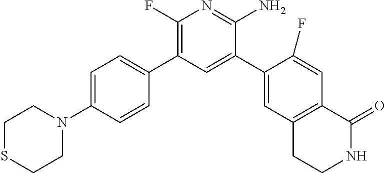 [SMILES: Nc1nc(F)c(-c2ccc(N3CCSCC3)cc2)cc1-c1cc2c(cc1F)C(=O)NCC2]

A procedure analogous to Example 177, Step 1, with 4-(4-bromophenyl)thiomorpholine (60 mg, 0.232 mmol), 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (131 mg, 0.325 mmol), [1,12-bis(diphenylphosphino)ferrocene]dichloropalladium(II) (23.81 mg, 0.033 mmol), Cs₂CO₃ (227 mg, 0.697 mmol), DME (3.5 mL) and water (1 mL) afforded the title compound as a beige solid (39 mg, 35%). ¹H NMR (500 MHz, DMSO-d₆) 5=8.19-8.07 (m, 1H), 7.65-7.56 (m, 2H), 7.47-7.34 (m, 3H), 7.02-6.90 (m, 2H), 6.25-6.11 (m, 2H), 3.65-3.52 (m, 4H), 3.44-3.40 (m, 2H), 2.95-2.89 (m, 2H), 2.73-2.60 (m, 4H); LCMS: [M+H]⁺=453.33.

### Example 348: (3aS,4S,6aR)-4-(5-(4-(4-(6-amino-2-fluoro-5-(1-oxo-1,2,3,4-tetrahydroisoquinolin-6-yl)pyridin-3-yl)phenoxy)piperidin-1-yl)-5-oxopentyl)tetrahydro-1H-thieno[3,4-d]imidazol-2(3H)-one (I-348)

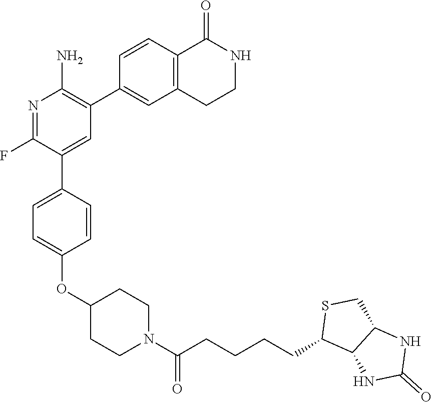 [SMILES: Nc1nc(F)c(-c2ccc(OC3CCN(C(=O)CCCC[C@@H]4SC[C@@H]5NC(=O)N[C@@H]54)CC3)cc2)cc1-c1ccc2c(c1)CCNC2=O]

To a solution of 6-(2-amino-6-fluoro-5-(4-(piperidin-4-yloxy)phenyl)pyridin-3-yl)-3,4-dihydroisoquinolin-1(2H)-one (45.7 mg, 0.106 mmol) in DMF (2 mL) was added Et₃N (0.015 ml, 0.106 mmol) at RT followed by Biotin-Osu (36.1 mg, 0.106 mmol). The reaction mixture was stirred at 24° C. for 3 h then dry loaded onto Celite® and purified by flash chromatography (0-20% DCM/MeOH) to afford the title compound (59.9 mg, 82%) as a white powder. ¹H NMR (500 MHz, DMSO-d₆) δ 9.37 (br s, 1H), 7.94 (br s, 1H), 7.90 (d, J=7.8 Hz, 1H), 7.60 (d, J=10.3 Hz, 1H), 7.50-7.42 (m, 4H), 7.03 (d, J=8.7 Hz, 2H), 6.43 (s, 1H), 6.35 (s, 1H), 6.23 (s, 2H), 5.75 (s, 1H), 4.69-4.60 (m, 1H), 4.35-4.27 (m, 1H), 4.17-4.11 (m, 1H), 3.91-3.81 (m, 1H), 3.75-3.66 (m, 1H), 3.43-3.37 (m, 2H), 3.28-3.21 (m, 1H), 3.14-3.00 (m, 3H), 2.95 (br t, J=6.4 Hz, 2H), 2.82 (dd, J=5.1, 12.4 Hz, 1H), 2.61-2.55 (m, 1H), 2.33 (br t, J=7.4 Hz, 2H), 2.03-1.94 (m, 1H), 1.93-1.85 (m, 1H), 1.68-1.57 (m, 2H), 1.51 (br d, J=7.7 Hz, 4H), 1.40-1.30 (m, 2H), 1.25-1.12 (m, 4H); LCMS: [M+H]⁺=659.58.

### Example 349: 6-(2-amino-6-fluoro-5-(4-(4-isopropylpiperazin-1-yl)phenyl)pyridin-3-yl)-7-chloro-3,4-dihydroisoquinolin-1(2H)-one (I-349)

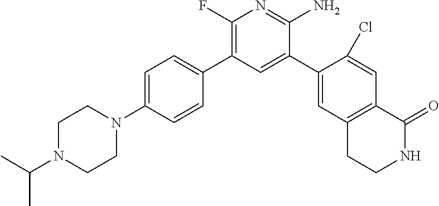 [SMILES: CC(C)N1CCN(c2ccc(-c3cc(-c4cc5c(cc4Cl)C(=O)NCC5)c(N)nc3F)cc2)CC1]

Step 1: 6-(2-amino-5-bromo-6-fluoropyridin-3-yl)-7-chloro-3,4-dihydroisoquinolin-1(2H)-one

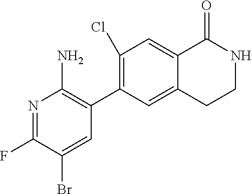 [SMILES: Nc1nc(F)c(Br)cc1-c1cc2c(cc1Cl)C(=O)NCC2]

To a degassed solution of 5-bromo-6-fluoro-3-iodopyridin-2-amine (309 mg, 0.976 mmol) and (7-chloro-1-oxo-1,2,3,4-tetrahydroisoquinolin-6-yl)boronic acid (200 mg, 0.887 mmol) in acetonitrile (6 ml), in a microwave vial, was added a solution of sodium carbonate monohydrate (231 mg, 1.86 mmol) in water (1.5 mL). The system was degassed and bis(triphenylphosphine)palladium(II) dichloride (74.7 mg, 0.106 mmol) was added. The reaction was further flushed with nitrogen and heated in the microwave at 80° C. for 3 h. The reaction was concentrated onto Celite® and purified by silica gel chromatography eluting with 0-100% EtOAc/Hexanes to give the product (62.6 mg, 19%) as a beige solid. LCMS: [M+H]+=370.08.

Step 2: 6-(2-amino-6-fluoro-5-(4-(4-isopropylpiperazin-1-yl)phenyl)pyridin-3-yl)-7-chloro-3,4-dihydroisoquinolin-1(2H)-one

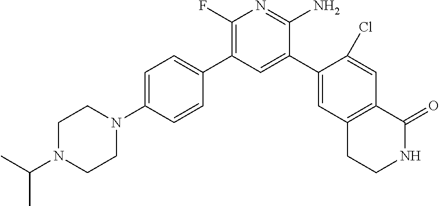 [SMILES: CC(C)N1CCN(c2ccc(-c3cc(-c4cc5c(cc4Cl)C(=O)NCC5)c(N)nc3F)cc2)CC1]

To a suspension of 6-(2-amino-5-bromo-6-fluoropyridin-3-yl)-7-chloro-3,4-dihydroisoquinolin-1(2H)-one (30 mg, 0.081 mmol), 4-(4-isopropylpiperazinyl)phenylboronic acid, pinacol ester (32.1 mg, 0.097 mmol) and Cs₂CO₃ (65.9 mg, 0.202 mmol) in a 3:1 mixture of DML (4 mL): water (1.3 mL) was added PdCl₂dppf (5.92 mg, 8.09 μmol). The mixture was flushed with nitrogen and heated in the microwave at 90° C. for 2 h. The reaction was concentrated onto Celite® and purified by silica gel chromatography eluting with 0-10% MeOH/DCM+1% NH₄OH, followed by reverse phase chromatography (C18, 0-100% ACN/H₂O). The desired fractions were then passed through an Isolute SCX-2 cation exchange resin cartridge eluting with 3% NH₃ in MeOH to give the title compound (16.8 mg, 42%) as a beige solid. ¹H NMR (500 MHz, DMSO-d₆) δ 8.12 (br. s., 1H), 7.90 (s, 1H), 7.51 (d, J=10.3 Hz, 1H), 7.40 (s, 1H), 7.36 (d, J=8.2 Hz, 2H), 6.95 (d, J=8.7 Hz, 2H), 6.07 (s, 2H), 3.40 (d, J=2.1 Hz, 2H), 3.14 (br. s., 4H), 2.92 (t, J=6.4 Hz, 2H), 2.65 (d, J=19.6 Hz, 1H), 2.58 (br. s., 4H), 1.01 (d, J=6.11 Hz, 6H); LCMS: [M+H]⁺=494.38.

### Example 350: 6-(2-amino-6-fluoro-5-(4-(1-(2-methoxyethyl)piperidin-4-yl)phenyl)pyridin-3-yl)-8-fluoro-3,4-dihydroisoquinolin-1(2H)-one (I-350)

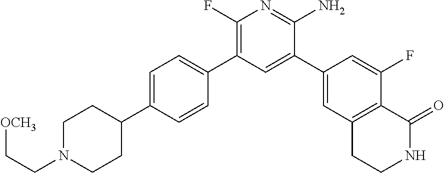 [SMILES: COCCN1CCC(c2ccc(-c3cc(-c4cc(F)c5c(c4)CCNC5=O)c(N)nc3F)cc2)CC1]

To a solution of 6-(2-amino-6-fluoro-5-(4-(piperidin-4-yl)phenyl)pyridin-3-yl)-8-fluoro-3,4-dihydroisoquinolin-1(2H)-one, trifluoroacetate (25 mg, 0.046 mmol) and N,N-diisopropylethylamine (0.079 ml, 0.456 mmol) in DMF (1 mL) was added 2-bromoethyl methyl ether (31.7 mg, 0.228 mmol). The reaction was stirred at RT overnight. The reaction was concentrated onto Celite® and purified by silica gel chromatography eluting with 0-10% MeOH/DCM+1% NH₄OH, followed by reverse phase chromatography (C18, 0-100% ACN/H₂O). The desired fractions were then passed through an Isolute SCX-2 cation exchange resin cartridge eluting with 3% NH₃ in MeOH to give the title compound (14.6 mg, 65%) as a white solid. ¹H NMR (500 MHz, DMSO-d₆) δ 7.99 (br. s., 1H), 7.67 (d, J=10.2 Hz, 1H), 7.47 (d, J=7.5 Hz, 2H), 7.23-7.33 (m, 4H), 6.39 (s, 2H), 3.45 (t, J=5.7 Hz, 2H), 3.24 (s, 3H), 2.99 (d, J=7.3 Hz, 2H), 2.93 (t, J=6.2 Hz, 2H), 2.1 (br. s., 2H), 1.71-1.79 (m, 2H), 1.61-1.70 (m, 2H); LCMS: [M+H]⁺=493.57.

### Example 351: (R)-6-(2-amino-6-fluoro-5-(4-(2-isopropylmorpholino)phenyl)pyridin-3-yl)-8-fluoro-3,4-dihydroisoquinolin-1(2H)-one (I-351)

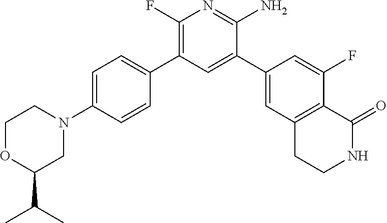 [SMILES: CC(C)[C@@H]1CN(c2ccc(-c3cc(-c4cc(F)c5c(c4)CCNC5=O)c(N)nc3F)cc2)CCO1]

A procedure analogous to Example 350 using 6-(2-amino-5-bromo-6-fluoropyridin-3-yl)-8-fluoro-3,4-dihydroisoquinolin-1(2H)-one (35 mg, 0.099 mmol), (R)-2-isopropyl-4-(4-(4,4,5,5-tetramethyl-1,3,2-dioxaborolan-2-yl)phenyl)morpholine (2.2 mL, 0.119 mmol, 17.5 mg/ml in ACN) and Cs₂CO₃ (80 mg, 0.247 mmol) in a 3:1 mixture of DME (2 mL): water (0.67 mL) and PdCl₂dppf (7.23 mg, 9.88 μmol) afforded the title compound (8.9 mg, 19%) as a tan solid. ¹H NMR (500 MHz, DMSO-d₆) δ 7.99 (br. s., 1H), 7.62 (d, J=10.2 Hz, 1H), 7.42 (d, J=8.0 Hz, 2H), 7.29 (s, 1H), 7.26 (d, J=12.0 Hz, 1H), 7.00 (d, J=8.8 Hz, 2H), 6.29 (s, 2H), 3.97 (dd, J=11.3, 2.2 Hz, 1H), 3.56-3.65 (m, 2H), 3.53 (d, J=11.9 Hz, 1H), 3.24 (ddd, J=10.1, 6.5, 2.2 Hz, 1H), 2.93 (t, J=6.2 Hz, 2H), 2.66 (td, J=11.8, 3.4 Hz, 1H), 2.44 (t, J=11.2 Hz, 1H), 1.73 (dq, J=13.4, 6.7 Hz, 1H), 0.96 (dd, J=6.7, 4.2 Hz, 6H); LCMS: [M+H]⁺=479.09.

### Example 352: 6-(2-amino-6-fluoro-5-(4-((1R,5S)-3-methyl-3-azabicyclo[3.1.0]hexan-1-yl)phenyl)pyridin-3-yl)-8-fluoro-3,4-dihydroisoquinolin-1(2H)-one (I-352)

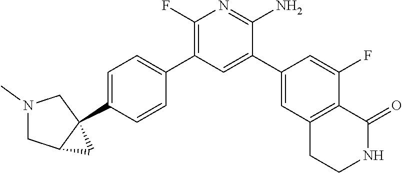 [SMILES: CN1C[C@H]2C[C@@]2(c2ccc(-c3cc(-c4cc(F)c5c(c4)CCNC5=O)c(N)nc3F)cc2)C1]

A procedure analogous to Example 350 using 6-(2-amino-5-bromo-6-fluoropyridin-3-yl)-8-fluoro-3,4-dihydroisoquinolin-1(2H)-one (35 mg, 0.099 mmol), (1R,5S)-3-methyl-1-(4-(4,4,5,5-tetramethyl-1,3,2-dioxaborolan-2-yl)phenyl)-3-azabicyclo[3.1.0]hexane (35.5 mg, 0.119 mmol) and Cs₂CO₃ (80 mg, 0.247 mmol) in a 3:1 mixture of DME (2 mL): water (0.67 ml) and PdCl₂dppf (7.23 mg, 9.88 μmol) afforded the title compound (16.4 mg, 37%) as a beige solid. ¹H NMR (500 MHz, DMSO-d₆) δ 7.99 (br. s., 1H), 7.65 (d, J=10.2 Hz, 1H), 7.46 (d, J=7.5 Hz, 2H), 7.29 (s, 1H), 7.26 (d, J=12.1 Hz, 1H), 7.18 (d, J=8.3 Hz, 2H), 6.39 (s, 2H), 3.28 (d, J=8.2 Hz, 1H), 3.00 (d, J=7.8 Hz, 1H), 2.93 (t, J=6.2 Hz, 2H), 2.42 (br. s., 1H), 2.31 (br. s., 3H), 1.77-1.85 (m, 1H), 1.35 (t, J=4.0 Hz, 1H), 0.77 (d, J=3.91 Hz, 1H); LCMS: [M+H]⁺=447.32.

### Example 353: 6-(2-amino-6-fluoro-5-(4-((1S,5R)-3-methyl-3-azabicyclo[3.1.0]hexan-1-yl)phenyl)pyridin-3-yl)-8-fluoro-3,4-dihydroisoquinolin-1(2H)-one (I-353)

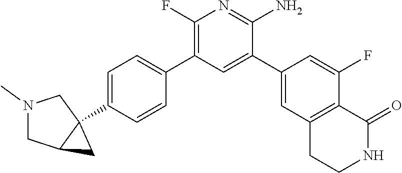 [SMILES: CN1C[C@@H]2C[C@]2(c2ccc(-c3cc(-c4cc(F)c5c(c4)CCNC5=O)c(N)nc3F)cc2)C1]

A procedure similar to Example 350 using 6-(2-amino-5-bromo-6-fluoropyridin-3-yl)-8-fluoro-3,4-dihydroisoquinolin-1(2H)-one (35 mg, 0.099 mmol), (1 S,5R)-3-methyl-1-(4-(4,4,5,5-tetramethyl-1,3,2-dioxaborolan-2-yl)phenyl)-3-azabicyclo[3.1.0]hexane (35.5 mg, 0.119 mmol) and Cs₂CO₃ (80 mg, 0.247 mmol) in a 3:1 mixture of DME (2 mL): water (0.67 mL) and PdCl₂dppf (7.23 mg, 9.88 μmol) afforded the title compound (14.7 mg, 33%) as an off-white solid. ¹H NMR (500 MHz, DMSO-d₆) δ 7.99 (br. s., 1H), 7.65 (d, J=10.0 Hz, 1H), 7.46 (d, J=7.5 Hz, 2H), 7.29 (s, 1H), 7.26 (d, J=12.0 Hz, 1H), 7.17 (d, J=8.3 Hz, 2H), 6.39 (s, 2H), 3.26 (d, J=8.4 Hz, 1H), 2.98 (d, J=8.7 Hz, 1H), 2.93 (t, J=6.2 Hz, 2H), 2.39 (d, J=6.1 Hz, 1H), 2.30 (s, 3H), 1.79 (dt, J=7.7, 3.8 Hz, 1H), 1.35 (t, J=4.0 Hz, 1H), 0.76 (dd, J=7.9, 3.7 Hz, 1H); LCMS: [M+H]⁺=447.26.

### Example 354: 6-(2-amino-6-fluoro-5-(4-(4-isopropylpiperazin-1-yl)phenyl)pyridin-3-yl)-8-methoxy-3,4-dihydroisoquinolin-1(2H)-one (I-354)

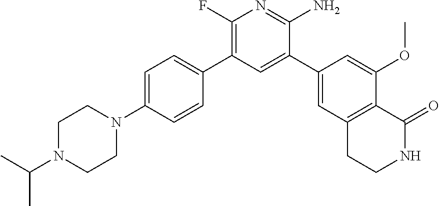 [SMILES: COc1cc(-c2cc(-c3ccc(N4CCN(C(C)C)CC4)cc3)c(F)nc2N)cc2c1C(=O)NCC2]

To a stirred solution of 6-(2-amino-6-fluoro-5-(4-(4-isopropylpiperazin-1-yl)phenyl)pyridin-3-yl)-8-fluoro-3,4-dihydroisoquinolin-1(2H)-one (25 mg, 0.052 mmol) in 1,4-dioxane (2 ml) was added sodium methoxide, 0.5M in MeOH (0.16 mL, 0.079 mmol). The mixture was stirred at RT overnight. The reaction was quenched with saturated aqueous NH₄Cl solution, then the volatiles were removed in vacuo. The material was diluted with water and the suspension was filtered. The crude material was concentrated onto Celite® and purified by reverse phase chromatography (C18, 0-100% MeCN—H₂O) to give the title compound (14.9 mg, 58%) as an off-white solid. ¹H NMR (500 MHz, DMSO-d₆) δ 7.69 (br. s., 1H), 7.61 (d, J=10.2 Hz, 1H), 7.39 (d, J=8.1 Hz, 2H), 7.04 (s, 1H), 6.98 (d, J=2.7 Hz, 2H), 6.96 (s, 1H), 6.20 (s, 2H), 3.81 (s, 3H), 3.23-3.28 (m, 2H), 3.10-3.17 (m, 4H), 2.84 (t, J=6.0 Hz, 2H), 2.67 (dt, J=13.0, 6.5 Hz, 1H), 2.54-2.60 (m, 4H), 1.00 (d, J=6.5 Hz, 6H); LCMS: [M+H]⁺=490.43.

### Example 355: 6-(2-amino-6-fluoro-5-(4-(4-isopropylpiperazin-1-yl)phenyl)pyridin-3-yl)-8-(dimethylamino)-3,4-dihydroisoquinolin-1(2H)-one (I-355)

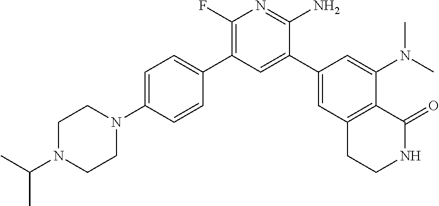 [SMILES: CC(C)N1CCN(c2ccc(-c3cc(-c4cc5c(c(N(C)C)c4)C(=O)NCC5)c(N)nc3F)cc2)CC1]

To a stirred solution of 6-(2-amino-6-fluoro-5-(4-(4-isopropylpiperazin-1-yl)phenyl)pyridin-3-yl)-8-fluoro-3,4-dihydroisoquinolin-1(2H)-one (25 mg, 0.052 mmol) in tetrahydrofuran (THF) (2.0 mL) was added K₂CO₃ (21.70 mg, 0.157 mmol) and dimethylamine, 2.0M in THF (0.79 mL, 1.57 mmol) solution. The mixture was stirred at 65° C. overnight. LCMS analysis showed ˜78% conversion of the starting material. The reaction was further heated at 80° C. overnight. The reaction was quenched with saturated aqueous NH₄Cl solution, then the volatiles were removed in vacuo. The material was diluted with water and the suspension was filtered. The crude material was concentrated onto Celite® and purified by reverse phase chromatography (C18, 0-100% MeCN—H₂O) to give the title compound (9.2 mg, 35%) as a beige solid. ¹H NMR (500 MHz, DMSO-d₆) δ 7.71 (br s, 1H), 7.57 (d, J=10.3 Hz, 1H), 7.39 (br d, J=8.1 Hz, 2H), 6.97 (br d, J=8.6 Hz, 2H), 6.84 (s, 1H), 6.75 (s, 1H), 6.11 (s, 2H), 3.27 (br d, J=4.5 Hz, 2H), 3.15 (br s, 4H), 2.86-2.77 (m, 8H), 2.59 (br s, 4H), 1.02 (br d, J=5.1 Hz, 6H); LCMS: [M+H]⁺=503.53.

### Example 356: 6-(2-amino-6-fluoro-5-(4-(4-isopropylpiperazin-1-yl)phenyl)pyridin-3-yl)-8-(methylamino)-3,4-dihydroisoquinolin-1(2H)-one (I-356)

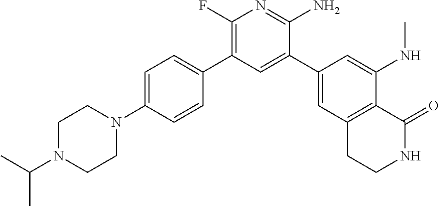 [SMILES: CNc1cc(-c2cc(-c3ccc(N4CCN(C(C)C)CC4)cc3)c(F)nc2N)cc2c1C(=O)NCC2]

To a stirred solution of 6-(2-amino-6-fluoro-5-(4-(4-isopropylpiperazin-1-yl)phenyl)pyridin-3-yl)-8-fluoro-3,4-dihydroisoquinolin-1(2H)-one (25 mg, 0.052 mmol) in 1,4-dioxane (2.0 mL) was added K₂CO₃ (21.70 mg, 0.157 mmol) and methylamine, 2.0 M in THF (0.79 mL, 1.57 mmol) solution. The mixture was stirred at 80° C. for 4 d. Additional methylamine, 2.0M in THF (2.0 ml) solution was added at the beginning of each day. The reaction was quenched with saturated aqueous NH₄Cl solution. The crude material was concentrated onto Celite® and purified by reverse phase chromatography (C18, 0-100% MeCN—H₂O) to afford the title compound (10.7 mg, 42%) as a beige solid. ¹H NMR (500 MHz, DMSO-d₆) δ 8.56 (q, J=4.6 Hz, 1H), 7.72 (br s, 1H), 7.56 (d, J=10.3 Hz, 1H), 7.38 (br d, J=8.3 Hz, 2H), 6.96 (d, J=8.7 Hz, 2H), 6.56 (s, 1H), 6.50 (s, 1H), 6.10 (s, 2H), 3.18-3.11 (m, 4H), 2.82 (br d, J=4.8 Hz, 5H), 2.70-2.64 (m, 1H), 2.60-2.55 (m, 4H), 1.00 (d, J=6.5 Hz, 6H); LCMS: [M+H]⁺=489.43

### Example 357: 8-amino-6-(2-amino-6-fluoro-5-(4-(4-isopropylpiperazin-1-yl)phenyl)pyridin-3-yl)-3,4-dihydroisoquinolin-1(2H)-one (I-357)

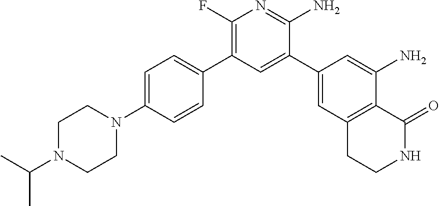 [SMILES: CC(C)N1CCN(c2ccc(-c3cc(-c4cc(N)c5c(c4)CCNC5=O)c(N)nc3F)cc2)CC1]

Step 1: 6-(2-amino-6-fluoro-5-(4-(4-isopropylpiperazin-1-yl)phenyl)pyridin-3-yl)-8-((2,4-dimethoxybenzyl)amino)-3,4-dihydroisoquinolin-1(2H)-one

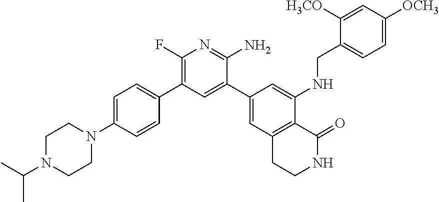 [SMILES: COc1ccc(CNc2cc(-c3cc(-c4ccc(N5CCN(C(C)C)CC5)cc4)c(F)nc3N)cc3c2C(=O)NCC3)c(OC)c1]

To a stirred solution of 6-(2-amino-6-fluoro-5-(4-(4-isopropylpiperazin-1-yl)phenyl)pyridin-3-yl)-8-fluoro-3,4-dihydroisoquinolin-1(2H)-one (45 mg, 0.094 mmol) in N,N-dimethylformamide (DMF) (1.0 ml) was added K₂CO₃ (39.1 mg, 0.283 mmol) followed by 2,4-dimethoxybenzylamine (158 mg, 0.942 mmol). The reaction was heated at 80° C. for 2 days. The reaction was quenched with saturated aqueous NH₄Cl solution, then the volatiles were removed in vacuo. The crude material was concentrated onto Celite® and purified by silica gel chromatography eluting with 0-10% MeOH/DCM+1% NH₄OH to give the product as a beige solid. LCMS: [M+H]⁺=625.61.

Step 2: 8-amino-6-(2-amino-6-fluoro-5-(4-(4-isopropylpiperazin-1-yl)phenyl)pyridin-3-yl)-3,4-dihydroisoquinolin-1(2H)-one

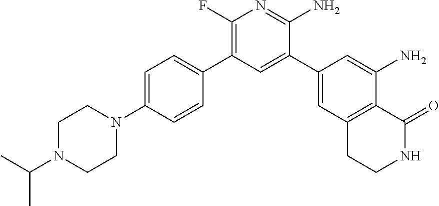 [SMILES: CC(C)N1CCN(c2ccc(-c3cc(-c4cc(N)c5c(c4)CCNC5=O)c(N)nc3F)cc2)CC1]

To a solution of the 6-(2-amino-6-fluoro-5-(4-(4-isopropylpiperazin-1-yl)phenyl)pyridin-3-yl)-8-((2,4-dimethoxybenzyl)amino)-3,4-dihydroisoquinolin-1(2H)-one in CH₂Cl₂ (1.0 mL) was added TFa (215 mg, 1.89 mmol). The mixture was stirred at RT for 1 h. The mixture was concentrated onto Celite® and purified by reverse phase chromatography (C18, 0-100% MeCN—H₂O). The desired fractions were then passed through an Isolute SCX-2 cation exchange resin cartridge eluting with 3% NH₃ in MeOH to give the title compound (8.4 mg, 19%) as an off-white solid. ¹H NMR (500 MHz, DMSO-d₆) δ 7.64 (br. s., 1H), 7.51 (d, J=10.2 Hz, 1H), 7.38 (d, J=8.0 Hz, 2H), 6.97 (d, J=8.8 Hz, 2H), 6.91 (br. s., 2H), 6.65 (s, 1H), 6.46 (s, 1H), 6.07 (s, 2H), 3.17 (br. s., 4H), 2.80 (t, J=6.4 Hz, 2H), 2.69-2.77 (m, 1H), 2.63 (br. s., 4H), 1.03 (d, J=6.4 Hz, 6H); LCMS: [M+H]⁺=475.39.

### Example 358: 8-(2-amino-6-fluoro-5-(4-(4-isopropylpiperazin-1-yl)phenyl)pyridin-3-yl)-3,4-dihydrobenzo[f][1,4]oxazepin-5(2H)-one (I-358)

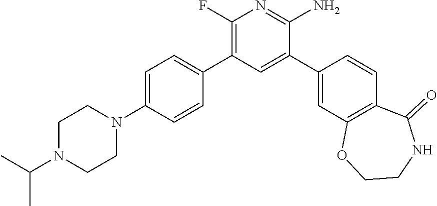 [SMILES: CC(C)N1CCN(c2ccc(-c3cc(-c4ccc5c(c4)OCCNC5=O)c(N)nc3F)cc2)CC1]

Step 1: 8-(4,4,5,5-tetramethyl-1,3,2-dioxaborolan-2-yl)-3,4-dihydrobenzo[f][1,4]oxazepin-5(2H)-one

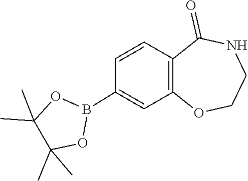 [SMILES: CC1(C)OB(c2ccc3c(c2)OCCNC3=O)OC1(C)C]

To a microwave vial charged with 8-bromo-3,4-dihydrobenzo[f][1,4]oxazepin-5(2H)-one (140 mg, 0.578 mmol), bis(pinacolato)diboron (162 mg, 0.636 mmol) and KOAc (170 mg, 1.74 mmol), was added anhydrous 1,4-dioxane (8.0 mL). The system was degassed then PdCl₂dppf (42.3 mg, 0.058 mmol) was added. The mixture was flushed with nitrogen then heated at 100° C. for 2.5 h. The reaction was diluted with acetonitrile, filtered through a pad of Celite, concentrated in vacuo and used without further purification for the next step. LCMS: [M+H]⁺=290.35.

Step 2: 8-(2-amino-5-bromo-6-fluoropyridin-3-yl)-3,4-dihydrobenzo[f][1,4]oxazepin-5(2H)-one

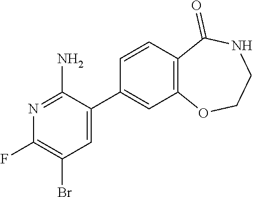 [SMILES: Nc1nc(F)c(Br)cc1-c1ccc2c(c1)OCCNC2=O]

To a solution of 5-bromo-6-fluoro-3-iodopyridin-2-amine (150 mg, 0.473 mmol) and 8-(4,4,5,5-tetramethyl-1,3,2-dioxaborolan-2-yl)-3,4-dihydrobenzo[f][1,4]oxazepin-5(2H)-one (164 mg, 0.568 mmol) in acetonitrile (8 mL) in a microwave vial, was added a solution of Na₂CO₃ H₂O (123 mg, 0.994 mmol) in water (2 mL). The system was degassed and bis(triphenylphosphine)palladium(II) dichloride (39.9 mg, 0.057 mmol) was added. The reaction was flushed with nitrogen and heated in the microwave at 80° C. for 3 h. The reaction was concentrated onto Celite® and purified by silica gel chromatography eluting with 0-100 % EtOAc/Hexanes to give the product (113 mg, 68%) as a beige solid. LCMS: [M+H]+=354.40.

Step 3: 8-(2-amino-6-fluoro-5-(4-(4-isopropylpiperazin-1-yl)phenyl)pyridin-3-yl)-3,4-dihydrobenzo[f][1,4]oxazepin-5(2H)-one

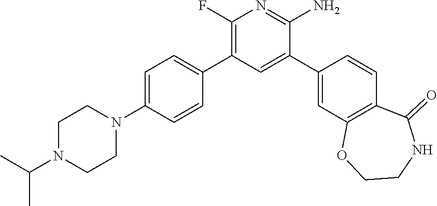 [SMILES: CC(C)N1CCN(c2ccc(-c3cc(-c4ccc5c(c4)OCCNC5=O)c(N)nc3F)cc2)CC1]

A procedure analogous to Example 350 using 8-(2-amino-5-bromo-6-fluoropyridin-3-yl)-3,4-dihydrobenzo[f][1,4]oxazepin-5(2H)-one (35 mg, 0.099 mmol), 4-(4-isopropylpiperazinyl)phenylboronic acid, pinacol ester (39.4 mg, 0.119 mmol) and Cs₂CO₃ (81 mg, 0.248 mmol) in a 3:1 mixture of DME (4 mL): water (1.3 mL) and PdCl₂dppf (7.27 mg, 9.94 μmol) afforded 8-(2-amino-6-fluoro-5-(4-(4-isopropylpiperazin-1-yl)phenyl)pyridin-3-yl)-3,4-dihydrobenzo[f][1,4]oxazepin-5(2H)-one, trifluoroacetate salt (10.3 mg, 18%) as a white solid. ¹H NMR (500 MHz, DMSO-d₆) δ 8.34 (t, J=5.1 Hz, 1H), 7.85 (d, J=8.2 Hz, 1H), 7.57 (d, J=10.2 Hz, 1H), 7.39 (d, J=8.0 Hz, 2H), 7.24 (dd, J=8.1, 1.5 Hz, 1H), 7.13 (d, J=1.5 Hz, 1H), 6.96 (d, J=8.8 Hz, 2H), 6.17 (s, 2H), 4.33 (t, J=4.7 Hz, 2H), 3.35-3.38 (m, 2H), 3.12-3.17 (m, 4H), 2.65-2.71 (m, 1H), 2.56-2.60 (m, 4H), 1.01 (d, J=6.5 Hz, 6H); LCMS: [M+H]⁺=476.39.

### Example 359: 6-(2-amino-5-(4-(1,1-dioxidothiomorpholino)phenyl)-6-fluoropyridin-3-yl)-8-fluoro-3,4-dihydroisoquinolin-1(2H)-one (I-359)

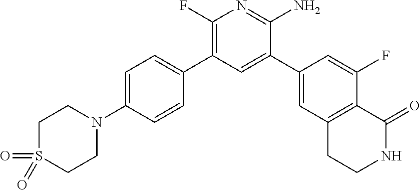 [SMILES: Nc1nc(F)c(-c2ccc(N3CCS(=O)(=O)CC3)cc2)cc1-c1cc(F)c2c(c1)CCNC2=O]

Step 1: 4-(4-(4,4,5,5-tetramethyl-1,3,2-dioxaborolan-2-yl)phenyl)thiomorpholine 1,1-dioxide

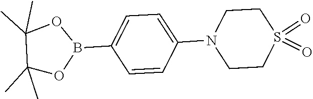 [SMILES: CC1(C)OB(c2ccc(N3CCS(=O)(=O)CC3)cc2)OC1(C)C]

To a round bottom flask charged with 4-(4-bromophenyl)thiomorpholine-1,1-dione (100 mg, 0.345 mmol), bis(pinacolato)diboron (96 mg, 0.379 mmol), KOAc (101 mg, 1.03 mmol), was added anhydrous 1,4-dioxane (10.0 mL). The system was degassed then PdCl₂dppf (25.2 mg, 0.034 mmol) was added. The mixture was flushed with nitrogen then heated at 100° C. on the stir plate for 1.5 hours. The reaction was diluted with acetonitrile, filtered through a pad of Celite®, concentrated in vacuo and used without further purification in the next step. LCMS: [M+H]⁺=338.44.

Step 2: 6-(2-amino-5-(4-(1,1-dioxidothiomorpholino)phenyl)-6-fluoropyridin-3-yl)-8-fluoro-3,4-dihydroisoquinolin-1(2H)-one

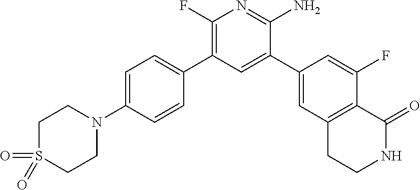 [SMILES: Nc1nc(F)c(-c2ccc(N3CCS(=O)(=O)CC3)cc2)cc1-c1cc(F)c2c(c1)CCNC2=O]

A procedure similar to Example 350 using 6-(2-amino-5-bromo-6-fluoropyridin-3-yl)-8-fluoro-3,4-dihydroisoquinolin-1(2H)-one (35 mg, 0.099 mmol), 4-(4-(4,4,5,5-tetramethyl-1,3,2-dioxaborolan-2-yl)phenyl)thiomorpholine 1,1-dioxide (43.3 mg, 0.128 mmol), Cs₂CO₃ (80 mg, 0.247 mmol) in a 3:1 mixture of DME (4 mL): water (1.3 mL-) and PdCl₂dppf (7.23 mg, 9.88 μmol) afforded the title compound (16.8 mg, 35%) as a white solid. 1H NMR (500 MHz, DMSO-d₆) δ 7.98 (br. s., 1H), 7.64 (d, J=10.2 Hz, 1H), 7.45 (d, J=8.1 Hz, 2H), 7.29 (s, 1H), 7.26 (d, J=12.4 Hz, 1H), 7.07 (d, J=8.9 Hz, 2H), 6.31 (s, 2H), 3.81 (br. s., 4H), 3.34 (d, J=2.9 Hz, 2H), 3.12 (d, J=4.2 Hz, 4H), 2.93 (t, J=6.2 Hz, 2H); LCMS: [M+H]⁺=485.36.

### Example 360: 6-(2-amino-6-fluoro-5-(4-(morpholinomethyl)phenyl)pyridin-3-yl)-8-fluoro-3,4-dihydroisoquinolin-1(2H)-one (I-360)

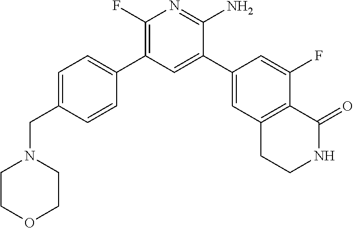 [SMILES: Nc1nc(F)c(-c2ccc(CN3CCOCC3)cc2)cc1-c1cc(F)c2c(c1)CCNC2=O]

A procedure similar to Example 350 using 6-(2-amino-5-bromo-6-fluoropyridin-3-yl)-8-fluoro-3,4-dihydroisoquinolin-1(2H)-one (30 mg, 0.085 mmol), 4-[4-(4,4,5,5-tetramethyl-1,3,2-dioxaborolan-2-yl)benzyl]morpholine (30.8 mg, 0.102 mmol), Cs₂CO₃ (69.0 mg, 0.212 mmol) in a 3:1 mixture of DME (4 mL): water (1.3 mL) and PdCl₂dppf (6.20 mg, 8.47 μmol) afforded the title compound (17.4 mg, 46%) as a beige solid. ¹H NMR (500 MHz, DMSO-d₆) δ 7.99 (br. s., 1H), 7.68 (d, J=9.9 Hz, 1H), 7.52 (d, J=7.7 Hz, 2H), 7.35 (d, J=8.1 Hz, 2H), 7.30 (s, 1H), 7.27 (d, J=12.0 Hz, 1H), 6.42 (s, 2H), 3.57 (m, 4H), 3.47 (s, 2H), 3.33-3.37 (m, 2H), 2.93 (t, J=6.1 Hz, 2H), 2.36 (br. s., 4H); LCMS: [M+H]+=451.39.

### Example 361: 6-(2-amino-6-fluoro-5-(3-fluoro-4-morpholinophenyl)pyridin-3-yl)-8-fluoro-3,4-dihydroisoquinolin-1(2H)-one (I-361)

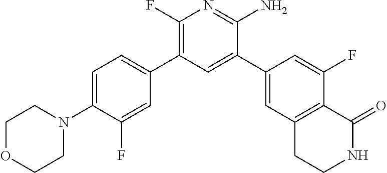 [SMILES: Nc1nc(F)c(-c2ccc(N3CCOCC3)c(F)c2)cc1-c1cc(F)c2c(c1)CCNC2=O]

A procedure analogous To Example 350 using 6-(2-amino-5-bromo-6-fluoropyridin-3-yl)-8-fluoro-3,4-dihydroisoquinolin-1(2H)-one (30 mg, 0.085 mmol), 3-fluoro-4-morpholinophenylboronic acid (22.87 mg, 0.102 mmol), Cs₂CO₃ (69.0 mg, 0.212 mmol) in a 3:1 mixture of DME (4 mL): water (1.3 mL) and PdCl₂dppf (6.20 mg, 8.47 μmol) afforded the title compound (19.7 mg, 51%) as a white solid. ¹H NMR (500 MHz, DMSO-d₆) δ 8.00 (br. s., 1H), 7.69 (d, J=10.2 Hz, 1H), 7.39 (d, J=14.4 Hz, 1H), 7.34 (d, J=8.4 Hz, H), 7.30 (s, 1H), 7.27 (d, J=12.4 Hz, 1H), 7.06 (t, J=8.9 Hz, 1H), 6.44 (s, 2H), 3.70-3.79 (m, 4H), 3.33-3.36 (m, 2H), 2.99-3.06 (m, 4H), 2.93 (t, J=6.2 Hz, 2H); LCMS: [M+H]⁺=455.34.

### Example 362: 7-(2-amino-6-fluoro-5-(4-(4-isopropylpiperazin-1-yl)phenyl)pyridin-3-yl)quinazolin-4(3H)-one (I-362)

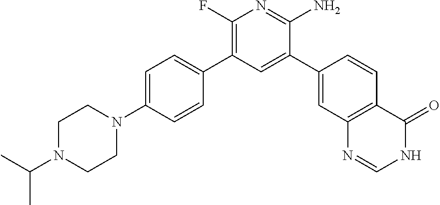 [SMILES: CC(C)N1CCN(c2ccc(-c3cc(-c4ccc5c(=O)[nH]cnc5c4)c(N)nc3F)cc2)CC1]

Step 1: (4-oxo-3,4-dihydroquinazolin-7-yl)boronic acid

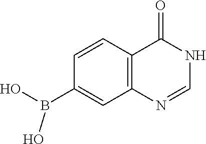 [SMILES: O=c1[nH]cnc2cc(B(O)O)ccc12]

To a round bottom flask charged with 7-bromoquinazolin-4(3 h)-one (790 mg, 3.51 mmol), bis(pinacolato)diboron (981 mg, 3.86 mmol), KOAc (1034 mg, 10.5 mmol), was added anhydrous 1,4-dioxane (40 mL). The system was degassed, then PdCl₂dppf (257 mg, 0.351 mmol) was added. The system was flushed with nitrogen and heated at 100° C. for 16 h. The reaction was diluted with CH₃CN, filtered through a pad of Celite, concentrated in vacuo and used as-is the next step assuming full conversion to the boronic acid. LCMS boronate: [M+H]⁺=273.43, LCMS boronic acid: [M+H]⁺=191.29.

Step 2: 7-(2-amino-5-bromo-6-fluoropyridin-3-yl)quinazolin-4(3H)-one

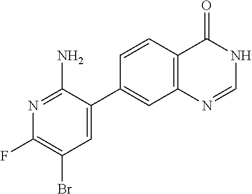 [SMILES: Nc1nc(F)c(Br)cc1-c1ccc2c(=O)[nH]cnc2c1]

To a solution of 5-bromo-6-fluoro-3-iodopyridin-2-amine (500 mg, 1.58 mmol) and (4-oxo-3,4-dihydroquinazolin-7-yl)boronic acid (10.8 ml, 1.89 mmol, 33.3 mg/mL in ACN) in acetonitrile (15 mL), in a microwave vial, was added a solution of Na₂CO₃ H₂O (411 mg, 3.31 mmol) in water (3.75 mL). The system was degassed and bis(triphenylphosphine)palladium(II) dichloride (133 mg, 0.189 mmol) was added. The reaction was flushed with nitrogen and heated in the microwave at 80° C. for 10 h. The reaction was concentrated onto Celite® and purified by silica gel chromatography eluting with 0-100% EtOAc/hexanes to give the product (166 mg, 31%) as a beige. LCMS: [M+H]⁺=335.24.

Step 3: 7-(2-amino-6-fluoro-5-(4-(4-isopropylpiperazin-1-yl)phenyl)pyridin-3-yl)quinazolin-4(3H)-one

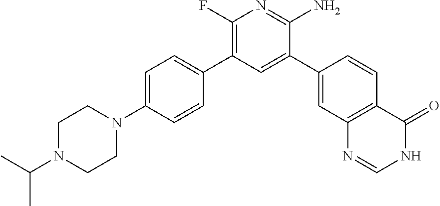 [SMILES: CC(C)N1CCN(c2ccc(-c3cc(-c4ccc5c(=O)[nH]cnc5c4)c(N)nc3F)cc2)CC1]

A procedure similar to Example 350 using 7-(2-amino-5-bromo-6-fluoropyridin-3-yl)quinazolin-4(3H)-one (35 mg, 0.104 mmol), 4-(4-isopropylpiperazinyl)phenylboronic acid, pinacol ester (41.4 mg, 0.125 mmol), Cs₂CO₃ (85 mg, 0.261 mmol) in a 3:1 mixture of DME (4 mL): water (1.3 mL) and PdCl₂dppf (7.64 mg, 10.44 μmol) afforded the title compound (15.9 mg, 33%) as a beige solid. ¹H NMR (500 MHz, DMSO-d₆) δ 12.26 (br. s., 1H), 8.17 (d, J=8.2 Hz, 1H), 8.13 (s, 1H), 7.76 (d, J=1.1 Hz, 1H), 7.65 (d, J=9.8 Hz, 2H), 7.42 (d, J=8.1 Hz, 2H), 6.98 (d, J=8.6 Hz, 2H), 6.24 (s, 2H), 3.16 (br. s., 4H), 2.89-3.01 (m, 1H), 2.63 (br. s., 4H), 1.04 (br. s., 6H); LCMS: [M+H]⁺=459.53.

### Example 363: 6-(2-amino-6-fluoro-5-(4-(4-isopropylpiperazin-1-yl)phenyl)pyridin-3-yl)phthalazin-1(2H)-one (I-363)

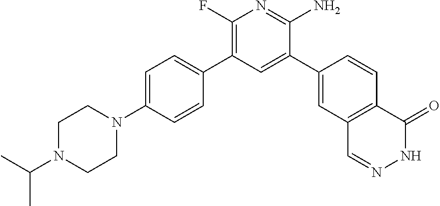 [SMILES: CC(C)N1CCN(c2ccc(-c3cc(-c4ccc5c(=O)[nH]ncc5c4)c(N)nc3F)cc2)CC1]

Step 1: (1-oxo-1,2-dihydrophthalazin-6-yl)boronic acid

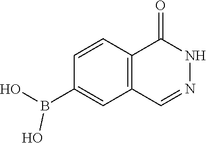 [SMILES: O=c1[nH]ncc2cc(B(O)O)ccc12]

A procedure analogous to Example 362, Step 1 using 6-bromophthalazin-1(2H)-one (500 mg, 2.222 mmol), bis(pinacolato)diboron (621 mg, 2.444 mmol), KOAc (654 mg, 6.67 mmol), anhydrous 1,4-dioxane (40 mL) and PdCl₂dppf (163 mg, 0.222 mmol) afforded the product. LCMS: Boronate: [M+H]⁺=273.30, LCMS Boronic acid: [M+H]⁺=191.23.

Step 2: 6-(2-amino-5-bromo-6-fluoropyridin-3-yl)phthalazin-1(2H)-one

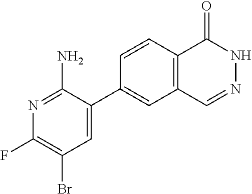 [SMILES: Nc1nc(F)c(Br)cc1-c1ccc2c(=O)[nH]ncc2c1]

A procedure analogous to Example 362, Step 2 using 5-bromo-6-fluoro-3-iodopyridin-2-amine (460 mg, 1.452 mmol) and (1-oxo-1,2-dihydrophthalazin-6-yl)boronic acid (12.8 mL, 1.89 mmol, 28.1 mg/ml in ACN) in CH₃CN (15 mL) and Na₂CO₃ H₂O (378 mg, 3.05 mmol) in water (3.75 mL) and bis(triphenylphosphine)palladium(II) dichloride (122 mg, 0.174 mmol) afforded the product (157 mg, 32%) as a beige solid. LCMS: [M+H]⁺=335.18.

Step 3: 6-(2-amino-6-fluoro-5-(4-(4-isopropylpiperazin-1-yl)phenyl)pyridin-3-yl)phthalazin-1(2H)-one

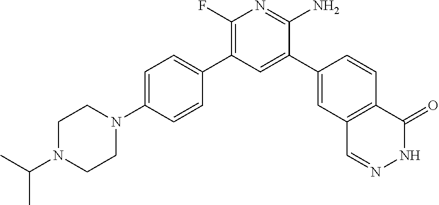 [SMILES: CC(C)N1CCN(c2ccc(-c3cc(-c4ccc5c(=O)[nH]ncc5c4)c(N)nc3F)cc2)CC1]

A procedure analogous to Example 350 using 6-(2-amino-5-bromo-6-fluoropyridin-3-yl)phthalazin-1(2H)-one (35 mg, 0.104 mmol), 4-(4-isopropylpiperazinyl)phenylboronic acid, pinacol ester (41.4 mg, 0.125 mmol), Cs₂CO₃ (85 mg, 0.261 mmol) in a 3:1 mixture of DME (4 mL): Water (1.3 mL) and PdCl₂dppf (7.64 mg, 10.44 μmol) afforded the title compound (19.3 mg, 40%) as a yellow solid. ¹H NMR (500 MHz, DMSO-d₆) δ 12.65 (s, 1H), 8.38 (s, 1H), 8.26 (d, J=8.2 Hz, 1H), 8.07 (d, J=1.4 Hz, 1H), 7.96 (dd, J=8.3, 1.53 Hz, 1H), 7.69 (d, J=10.2 Hz, 1H), 7.42 (d, J=8.0 Hz, 2H), 6.97 (d, J=8.8 Hz, 2H), 6.33 (s, 2H), 3.15 (br. s., 4H), 2.65-2.72 (m, 1H), 2.59 (br. s., 4H), 1.01 (d, J=6.2 Hz, 6H); LCMS: [M+H]⁺=459.47.

### Example 364: 6-(2-amino-6-fluoro-5-(4-(4-isopropylpiperazin-1-yl)phenyl)pyridin-3-yl)isoquinolin-1(2H)-one (I-364)

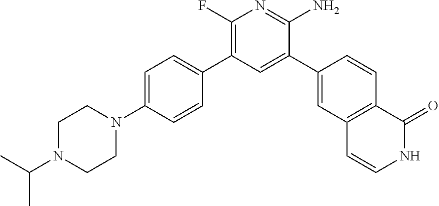 [SMILES: CC(C)N1CCN(c2ccc(-c3cc(-c4ccc5c(=O)[nH]ccc5c4)c(N)nc3F)cc2)CC1]

Step 1: 6-(2-amino-5-bromo-6-fluoropyridin-3-yl)isoquinolin-1(2H)-one

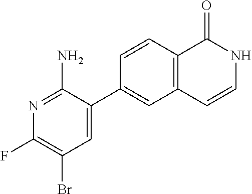 [SMILES: Nc1nc(F)c(Br)cc1-c1ccc2c(=O)[nH]ccc2c1]

A procedure analogous to Example 362 Step 2 using 5-bromo-6-fluoro-3-iodopyridin-2-amine (140 mg, 0.442 mmol) and 6-(tetramethyl-1,3,2-dioxaborolan-2-yl)-2H-isoquinolin-1-one (132 mg, 0.486 mmol) in CH₃CN (5 mL) Na₂CO₃ H₂O (115 mg, 0.928 mmol) in water (1.25 mL) and bis(triphenylphosphine)palladium(II) dichloride (37.2 mg, 0.053 mmol) afforded the product (76 mg, 52%) as a beige solid. LCMS: [M+H]⁺=334.24.

Step 2: 6-(2-amino-6-fluoro-5-(4-(4-isopropylpiperazin-1-yl)phenyl)pyridin-3-yl)isoquinolin-1(2H)-one

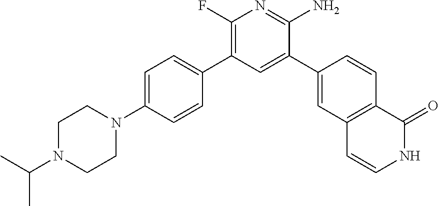 [SMILES: CC(C)N1CCN(c2ccc(-c3cc(-c4ccc5c(=O)[nH]ccc5c4)c(N)nc3F)cc2)CC1]

A procedure similar to Example 350 using 6-(2-amino-5-bromo-6-fluoropyridin-3-yl)isoquinolin-1(2H)-one (30 mg, 0.090 mmol), 4-(4-isopropylpiperazinyl)phenylboronic acid, pinacol ester (35.6 mg, 0.108 mmol), Cs₂CO₃ (73.1 mg, 0.224 mmol) in a 3:1 mixture of DME (4 mL): water (1.3 mL) and PdCl₂dppf (6.57 mg, 8.98 μmol) afforded the title compound (15.3 mg, 37%) as a beige solid. ¹H NMR (500 MHz, DMSO-d₆) δ 11.23 (d, J=4.3 Hz, 1H), 8.23 (d, J=8.3 Hz, 1H), 7.77 (s, 1H), 7.64 (d, J=10.2 Hz, 1H), 7.59 (d, J=7.3 Hz, 1H), 7.41 (d, J=8.1 Hz, 2H), 7.20 (t, J=6.4 Hz, 1H), 6.97 (d, J=8.6 Hz, 2H), 6.58 (d, J=7.1 Hz, 1H), 6.20 (br. s., 2H), 3.11-3.20 (m, 4H), 2.66-2.75 (m, 1H), 2.54-2.65 (m, 4H), 1.02 (d, J=4.7 Hz, 6H); LCMS: [M+H]⁺=458.53.

### Example 365: 6′-(2-amino-6-fluoro-5-(4-(4-isopropylpiperazin-1-yl)phenyl)pyridin-3-yl)-2′,3′-dihydro-1′H-spiro[cyclopropane-1,4′-isoquinolin]-1′-one (I-365)

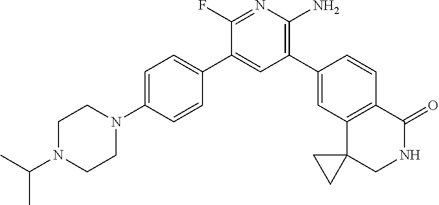 [SMILES: CC(C)N1CCN(c2ccc(-c3cc(-c4ccc5c(c4)C4(CC4)CNC5=O)c(N)nc3F)cc2)CC1]

Step 1: 6-bromo-1-methylene-2,3-dihydro-1H-indene

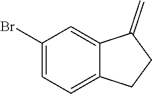

Methytriphenyl phosphonium bromide (101.12 g, 284.27 mmol) was dissolved in tetrahydrofuran (500 mL) followed by addition of potassium tert-butoxide (31.9 g, 284.27 mmol). Upon completion of the addition, the mixture was stirred at RT for 30 minutes. 6-Bromo-indan-1-one (20 g, 94.71 mmol) was dissolved in tetrahydrofuran (100 mL) stirring under nitrogen, treated with the ylide and the resulting mixture was stirred at RT for 1 h. The mixture was diluted with water (300 mL) and extracted with DCM (3×500 mL). The combined organic layer was back-washed with brine solution (2×500 mL), dried over Na₂SO₄ and concentrated under reduced pressure. The resulting crude product was purified by column chromatography over silica gel (100-200 mesh) and the product was eluted in 0-10% EtOAc in hexanes to afford the product (19 g, 95.9% yield) as a yellow oil. ¹H NMR (400 MHz, Chloroform-d) δ 7.65 (s, 1H), 7.36 (d, J=7.3 Hz, 1H), 7.17 (d, J=8.1 Hz, 1H), 5.38 (s, 1H), 5.11 (s, 1H), 3.01-2.93 (m, 2H), 2.89-2.81 (m, 2H).

Step 2: 6′-bromo-2′,3′-dihydrospiro[cyclopropane-1,1′-indene

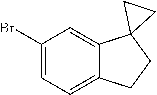 [SMILES: Brc1ccc2c(c1)C1(CC2)CC1]

The 1 M solution of diethyl zinc in hexanes (363.6 mL, 363.3 mmol) was added to a reaction vessel containing DCM (318 mL) and the mixture was cooled to 0° C. TFA (41.4 g, 363.3 mmol) was added and the reaction was further stirred at 0° C. for 15 minutes. To the cooled solution, diiodomethane (97 g, 363.3 mmol) was added and mixture was stirred for an additional 15 minutes at 0° C. After 15 min, a solution of 6-bromo-1-methylene-2,3-dihydro-1H-indene (19 g, 90.87 mmol) in DCM (100 mL) was added and reaction mass was gradually warm to RT and stirred for 16 h. The reaction was quenched with a sat. aq. NH₄Cl (500 mL) and extracted with DCM (3×300 mL). The combined organic layers were back-washed with brine solution (2×200 mL), dried over Na₂SO₄ and concentrated under reduced pressure. The resulting crude product was purified by column chromatography over silica gel (100-200 mesh) eluting with hexanes to afford the title compound (16 g, 78.9% yield) as a clear oil. ¹H NMR (400 MHz, Chloroform-d) δ 7.22 (dd, J=7.9, 1.9 Hz, 1H), 7.05 (d, J=7.8 Hz, 1H), 6.78 (d, J=1.8 Hz, 1H), 2.99 (t, J=7.5 Hz, 2H), 2.06-2.20 (t, 2H), 0.83-1.03 (m, 5H).

Step 3: Synthesis of 6′-bromospiro[cyclopropane-1,1′-inden]-3′(2′H)-one

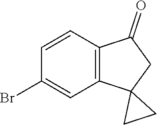 [SMILES: O=C1CC2(CC2)c2cc(Br)ccc21]

To a solution of 6′-bromo-2′,3′-dihydrospiro[cyclopropane-1,1′-indene (16 g, 71.71 mmol) in acetone (224 mL) and 1.5 M aq. MgSO₄ (80 mL) was added KMnO₄ (12.4 g, 78.5 mmol) and the reaction was stirred overnight at room temperature. After completion, the reaction was filtered over celite and concentrated. The crude was diluted with EtOAc (200 mL), washed with brine solution (2×100 mL), dried over Na₂SO₄ and concentrated under reduced pressure. The resulting crude product was purified by column chromatography over silica gel (100-200 mesh) eluting with 0-10% EtOAc in hexanes to give the product (10 g, 58.81%) as a white solid. LCMS: [M+2H]⁺=238.1.

Step 4: (Z)-6′-bromospiro[cyclopropane-1,1′-inden]-3′(2′H)-one oxime

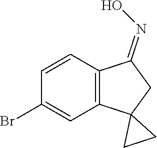 [SMILES: O/N=C1/CC2(CC2)c2cc(Br)ccc21]

A solution of 6′-bromospiro[cyclopropane-1,1′-inden]-3′(2′H)-one (10 g, 42.18 mmol), hydroxylamine HCl (5.5 g, 79.79 mmol) and NaOAc (20.3 g, 247.7 mmol) in MeOH (800 mL) was stirred at RT for 16 h. The reaction mixture was concentrated, slurried with water (40 mL), sonicated and filtered. The precipitate was dried under high vacuum to give the product (8 g, 75.2% yield) as a white solid. LCMS: [M+2H]⁺=253.1.

Step 5: (Z)-6′-bromospiro[cyclopropane-1,1′-inden]-3′(2′H)-one O-methylsulfonyl oxime

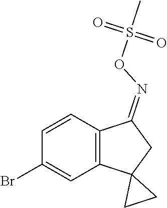 [SMILES: CS(=O)(=O)O/N=C1/CC2(CC2)c2cc(Br)ccc21]

A cooled solution of (E)-6′-bromospiro[cyclopropane-1,1′-inden]-3′(2′H)-one oxime (8 g, 31.73 mmol) in THF (240 mL) at 0° C., was added triethylamine (6.4 g, 63.46 mmol) followed by slow addition of MsCI (5.4 g, 47.6 mmol). The reaction was stirred at 0° C. for 30 min, and after 30 min the reaction was concentrated and the residue was triturated with methanol (4-5 mL) and dried to afford the product (5.5 g, 52.5% yield) as white solid. LCMS: [M+2H]⁺=331.2.

Step 6: 6′-bromo-2′,3′-dihydro-1′H-spiro[cyclopropane-1,4′-isoquinolin]-1′-onebenzoate

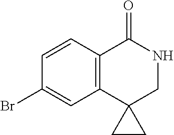 [SMILES: O=C1NCC2(CC2)c2cc(Br)ccc21]

To a cooled solution of (E)-6′-bromospiro[cyclopropane-1,1′-inden]-3′(2′H)-one-O-methylsulfonyl oxime (5.5 g, 16.66 mmol) in DCM (330 mL) at 0° C. was added BF₃MeOH (5.2 g, 36.66 mmol). TiCl₄(4.4 g, 23.33 mmol) was then added slowly and the reaction was stirred at 0° C. for 4 h. The reaction was quenched with water (50 mL) and extracted with DCM (2×100 mL). The combined organics were dried over Na₂SO₄ and concentrated under reduced pressure. The resulting crude product was purified by column chromatography over silica gel (100-200 mesh) eluting with 50-100% EtOAc in hexanes to afford the product (1 g, 23.8%) as white solid. LCMS: [M+2H]⁺=253.1.

Step 7: 6′-(4,4,5,5-tetramethyl-1,3,2-dioxaborolan-2-yl)-2′,3′-dihydro-1′H-spiro[cyclopropane-1,4′-isoquinolin]-1′-one

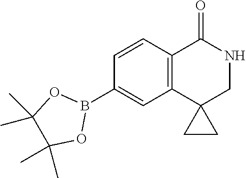 [SMILES: CC1(C)OB(c2ccc3c(c2)C2(CC2)CNC3=O)OC1(C)C]

Prepared from 6′-bromo-2′,3′-dihydro-1′H-spiro[cyclopropane-1,4′-isoquinolin]-1′-one (0.95 g, 3.77 mmol) to give a mixture of the boronate and boronic acid which was used in the next step without further purification. LCMS: [M+H]⁺ 218.21 Boronic acid, 300.28 Boronate.

Step 8: 6′-(2-amino-5-bromo-6-fluoropyridin-3-yl)-2′,3′-dihydro-1′H-spiro[cyclopropane-1,4′-isoquinolin]-1′-one (Prophetic Intermediate)

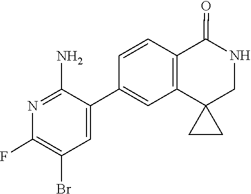 [SMILES: Nc1nc(F)c(Br)cc1-c1ccc2c(c1)C1(CC1)CNC2=O]

Can be prepared from 5-bromo-6-fluoro-3-iodopyridin-2-amine mmol) and 6′-(4,4,5,5-tetramethyl-1,3,2-dioxaborolan-2-yl)-2′,3′-dihydro-1′H-spiro[cyclopropane-1,4′-isoquinolin]-1′-one in a manner analogous to Example 362, Step 2 to give the product.

Step 9: 6′-(2-amino-6-fluoro-5-(4-(4-isopropylpiperazin-1-yl)phenyl)pyridin-3-yl)-2′,3′-dihydro-1′H-spiro[cyclopropane-1,4′-isoquinolin]-1′-one (Prophetic)

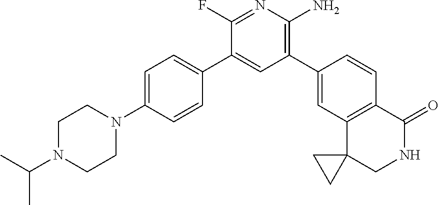 [SMILES: CC(C)N1CCN(c2ccc(-c3cc(-c4ccc5c(c4)C4(CC4)CNC5=O)c(N)nc3F)cc2)CC1]

Can be prepared from 4-(4-isopropylpiperazinyl)phenylboronic acid and 6′-(2-amino-5-bromo-6-fluoropyridin-3-yl)-2′,3′-dihydro-1′H-spiro[cyclopropane-1,4′-isoquinolin]-1′-one in a manner analogous to Example 362 Step 3 to give the title compound.

### Example 366: (S)-6-(2-amino-6-fluoro-5-(3-(pyrrolidin-2-yl)-4-(tetrahydro-2H-pyran-4-yl)phenyl)pyridin-3-yl)-3,4-dihydroisoquinolin-1(2H)-one (I-367)

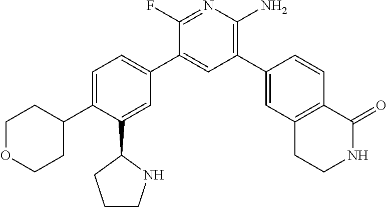 [SMILES: Nc1nc(F)c(-c2ccc(C3CCOCC3)c([C@@H]3CCCN3)c2)cc1-c1ccc2c(c1)CCNC2=O]

Step 1: tert-butyl (S)-2-(5-(6-amino-2-fluoro-5-(1-oxo-1,2,3,4-tetrahydroisoquinolin-6-yl)pyridin-3-yl)-2-(tetrahydro-2H-pyran-4-yl)phenyl)pyrrolidine-1-carboxylate

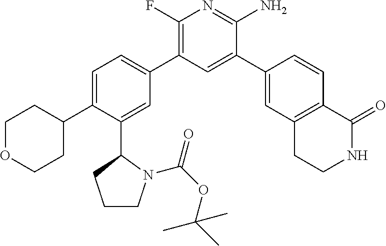 [SMILES: CC(C)(C)OC(=O)N1CCC[C@H]1c1cc(-c2cc(-c3ccc4c(c3)CCNC4=O)c(N)nc2F)ccc1C1CCOCC1]

A microwave vial containing a mixture of tert-butyl (S)-2-(5-bromo-2-(tetrahydro-2H-pyran-4-yl)phenyl)pyrrolidine-1-carboxylate (50 mg, 0.122 mmol, prepared as described in WO 202019351), tetrakis(triphenylphosphine)palladium(0) (16.9 mg, 0.015 mmol), 6-(2-amino-6-fluoro-5-(4,4,5,5-tetramethyl-1,3,2-dioxaborolan-2-yl)pyridin-3-yl)-3,4-dihydroisoquinolin-1(2H)-one (60.7 mg, 0.158 mmol) and Na₂CO₃—H₂O (45.3 mg, 0.366 mmol) in methyl tetrahydrofuran (4 mL) was degassed with Argon. Water (0.5 mL) was added and the reaction mixture was heated in a microwave reactor at 85° C. for 1 h. The reaction mixture was partitioned between water (3 mL) and EtOAc (3 mL), the organic phase was separated, the aqueous phase was extracted with EtOAc (3 mL), the combined organic phase was washed with brine (3 mL), dried over Na₂SO₄, concentrated onto Celite® and purified by column chromatography (0-100% EtOAc in hexanes) to yield the title compound (42 mg, 59%) as a beige solid. LCMS [M+H]⁺=587.7 HPLC purity 95%.

Step 2: (S)-6-(2-amino-6-fluoro-5-(3-(pyrrolidin-2-yl)-4-(tetrahydro-2H-pyran-4-yl)phenyl)pyridin-3-yl)-3,4-dihydroisoquinolin-1(2H)-one (I-367)

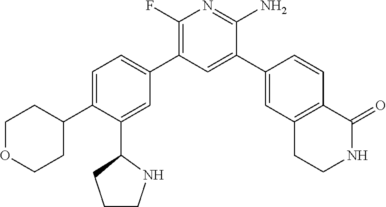 [SMILES: Nc1nc(F)c(-c2ccc(C3CCOCC3)c([C@@H]3CCCN3)c2)cc1-c1ccc2c(c1)CCNC2=O]

To a solution of tert-butyl (S)-2-(5-(6-amino-2-fluoro-5-(1-oxo-1,2,3,4-tetrahydroisoquinolin-6-yl)pyridin-3-yl)-2-(tetrahydro-2H-pyran-4-yl)phenyl)pyrrolidine-1-carboxylate (42 mg, 0.072 mmol) in DCM (2 mL), was added TFA (0.493 mL, 6.44 mmol) at RT and the reaction mixture was stirred at RT for 1.5 h. The reaction mixture was concentrated, and the residue was purified by preparatory HPLC using water (containing 0.1% formic acid)/acetonitrile (containing 0.1% formic acid) gradient. The combined fractions were mixed with 10% K₂CO₃ solution, extracted with CHCl₃IPA (4:1) solvent mixture (x 4), the combined organic layers were dried over Na₂SO₄ and concentrated to yield the title compound as a white solid (18 mg, 49%). ¹H NMR (500 MHz, METHANOL-d₄) δ 7.94 (d, J=8.0 Hz, 1H), 7.58-7.68 (m, 1H), 7.50 (s, 1H), 7.41 (d, J=8.1 Hz, 1H), 7.30-7.38 (m, 2 H), 7.27 (d, J=8.2 Hz, 1H), 4.42 (t, J=8.0 Hz, 1H), 3.90-4.02 (m, 2H), 3.52 (t, J=11.6 Hz, 2H), 3.41-3.47 (m, 2H), 3.05-3.16 (m, 2H), 2.88-2.98 (m, 3H), 2.12-2.25 (m, 1H), 1.73-1.98 (m, 4H), 1.54-1.69 (m, 3H). LCMS: [M+H]⁺=487.55.

Compounds 1-368, 1-369 and 1-370 were prepared following a procedure similar to Example 366 (1-367).

Compounds 1-367 to 1-425 were prepared following procedures as outlined in the Schemes above and described in Examples 1-1 to 1-366.

### Example 367: 6-(2-amino-6-fluoro-5-(4-(4-isopropylpiperazin-1-yl)phenyl)pyridin-3-yl)-4-methylisoquinolin-1(2H)-one (I-378)

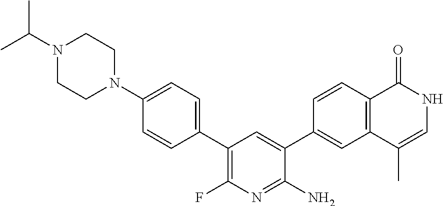 [SMILES: Cc1c[nH]c(=O)c2ccc(-c3cc(-c4ccc(N5CCN(C(C)C)CC5)cc4)c(F)nc3N)cc12]

Prepared from (4-methyl-1-oxo-1,2-dihydroisoquinolin-6-yl)boronic acid (48.3 mg, 0.238 mmol), and 3-bromo-6-fluoro-5-(4-(4-isopropylpiperazin-1-yl)phenyl)pyridin-2-amine (72 mg, 0.183 mmol) to afford the title compound (11.3 mg, 13% yield) as a white solid. 1H NMR (500 MHz, DMSO-d6) δ ppm 11.07 (d, J=5.50 Hz, 1H), 8.28 (d, J=8.31 Hz, 1H), 7.72 (s, 1H), 7.66 (d, J=10.15 Hz, 1H), 7.62 (dd, J=8.25, 1.28 Hz, 1H), 7.41 (d, J=7.95 Hz, 2H), 7.03 (d, J=4.89 Hz, 1H), 6.97 (d, J=8.93 Hz, 2H), 6.22 (s, 2H), 3.15 (br. s., 4H), 2.68 (br. s., 1H), 2.58 (br. s., 4H), 2.24 (s, 3H), 1.01 (d, J=6.48 Hz, 6H); LCMS: [M+H]+=472.40

### Example 368: 6-(2-amino-6-fluoro-5-(4-(4-isopropylpiperazin-1-yl)phenyl)pyridin-3-yl)-4-fluoro-3-methylisoquinolin-1(2H)-one (I-381)

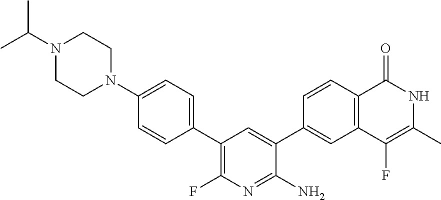 [SMILES: Cc1[nH]c(=O)c2ccc(-c3cc(-c4ccc(N5CCN(C(C)C)CC5)cc4)c(F)nc3N)cc2c1F]

Step 1: 6-bromo-4-fluoro-3-methylisoquinolin-1(2H)-one

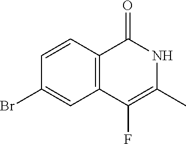

A vial was charged with 6-bromo-3-methyl-2H-isoquinolin-1-one (1000 mg, 4.20 mmol) and Selectfluor™ fluorinating reagent >95% in F+active (1711 mg, 4.83 mmol). Methanol (15 ml) and acetonitrile (15 ml) were added, and the reaction was stirred at room temperature for 5 days. The white suspension was filtered, washing with water, and dried under vacuum to the product (903 mg, 84% yield) as a white solid. LCMS: [M+H]⁺=256.15.

Step 2 1-(4-Bromophenyl)-4-isopropylpiperazine

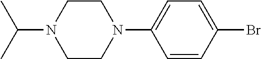 [SMILES: CC(C)N1CCN(c2ccc(Br)cc2)CC1]

To a solution of 1-(4-bromophenyl)-piperazine (10 g, 41.4 mmol) and anhydrous acetone (2.4 g, 41.4 mmol) in ethylene dichloride (100 mL), was added NaBH(OAc)₃ (13 g 62.1 mmol) and resultant reaction mass was stirred at room temperature for 1 h. After 1 h, another portion of anhydrous acetone (2.4 g, 41.4 mmol) was added and the reaction was stirred at room temperature for 16 h. After 16 h another portion of acetone (1.2 g, 20.7 mmol) was added and the reaction was further stirred for 6 h. The mixture was treated with 1N NaOH solution (100 mL) and extracted with diethyl ether (2×200 mL). The combined organic layer was washed with brine solution (100 mL), dried over anhydrous Na₂SO₄ and concentrated under vacuum to get crude. The crude was purified by column chromatography and the product was eluted in 4% MeOH in CH₂Cl₂ to afford the product (6 g, 51%) as a yellow solid. LCMS: [M+2H]⁺=285.27 Step 3: 6-flouro-5-iodopyridin-2-amine

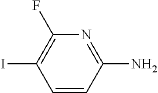

A solution of 6-flouro-5-iodopyridin-2-amine (5 g, 44.59 mmol) and N-iodosuccinimide (10.88 g, 48.4 mmol) in acetonitrile (100 mL) was stirred at 0° C. for 3 h. After completion of the reaction, reaction mass was concentrated and the residue was purified by column chromatography and the product was eluted in 10% MeOH in CH₂Cl₂ to afford the title compound (7.5 g, 31.51 mmol, 75%) as a brownish yellow solid. LCMS: [M+H]⁺=239.09 Step 4: 1-isopropyl-4-(4-(4,4,5,5-tetramethyl-1,3,2-dioxaborolan-2-yl)phenyl) piperazine

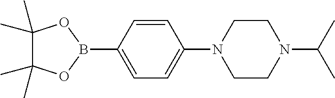 [SMILES: CC(C)N1CCN(c2ccc(B3OC(C)(C)C(C)(C)O3)cc2)CC1]

1-(4-Bromophenyl)-4-isopropylpiperazine (4.3 g, 15.19 mmol) was dissolved in degassed toluene (43 mL). To this, potassium acetate (5.88 g, 60 mmol), bis(pinacolato)diboron (6.5 g, 25.67 mmol) and Pd(dppf)Cl₂.DCM complex (1.22 g, 1.44 mmol) were added under nitrogen atmosphere and the reaction mass was stirred at 110° C. for 4 h. The volatiles were removed under reduced pressure to give crude product (4.2 g) as a black solid which was used as such in next step without further purification. LCMS: [M+H]+=331.46.

Step 5: 6-fluoro-5-(4-(4-isopropylpiperazin-1-yl)phenyl)pyridine-2-amine

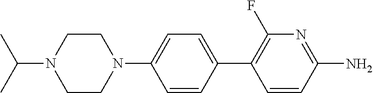 [SMILES: CC(C)N1CCN(c2ccc(-c3ccc(N)nc3F)cc2)CC1]

To a degassed solution of 1-isopropyl-4-(4-(4,4,5,5-tetramethyl-1,3,2-dioxaborolan-2-yl) phenyl) piperazine (5 g, 15.15 mmol) in dioxane (25 mL) and water (25 mL), potassium carbonate (5.2 g, 37.75 mmol) and Int-4 (3.96 g, 16.66 mmol) were added and the reaction mass was degassed for 10 min with N₂. To this, tetrakis (0.875 g, 0.75 mmol) was added under nitrogen atmosphere and reaction mass was stirred at 120° C. for 4 h. After completion of reaction, the reaction mass was diluted with water (100 mL) and extracted with EtOAc (3×100 mL). The combined organic layer was dried over anhydrous Na₂SO₄, concentrated under vacuum. The crude material was purified by column chromatography and the product was eluted in 4% MeOH in DCM to afford the product (3.25 g, 68%) as a brown solid. LCMS: [M+H]⁺=315.60.

Step 6: 3-bromo-6-fluoro-5-(4-(4-isopropylpiperazin-1-yl)phenyl)pyiridin-2-amine

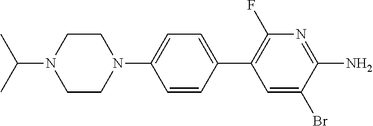 [SMILES: CC(C)N1CCN(c2ccc(-c3cc(Br)c(N)nc3F)cc2)CC1]

The mixture of 6-fluoro-5-(4-(4-isopropylpiperazin-1-yl)phenyl)pyridine-2-amine (6.7 g, 21.33 mmol) and NBS (3.77 g, 21.33 mmol) in DMF (60 mL) was stirred at room temperature 6 h. To this, further NBS (1.88 g, 10.66 mmol) was added and the reaction mass was stirred at room temperature for 16 h. After 16 h, as SM was not consumed completely therefore further NBS (1.13 g, 6.4 mmol) was added and reaction mass was stirred at RT for 3 h. After completion of the reaction, the reaction mass was diluted with saturated sodium bicarbonate solution (200 mL) and product was extracted with EtOAc (3×150 mL). The combined organic was dried over anhydrous Na₂SO₄, concentrated under vacuum and purified by column chromatography. The product was eluted in 50-100% EtOAc in hexanes to afford the product (1.3 g, 15%) as a brownish yellow solid. LCMS: [M+2H]⁺=395.5.

Step 7: 6-fluoro-5-(4-(4-isopropylpiperazin-1-yl)phenyl)-3-(4,4,5,5-tetramethyl-1,3,2-dioxaborolan-2-yl)pyridin-2-amine

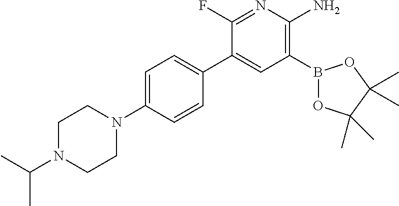 [SMILES: CC(C)N1CCN(c2ccc(-c3cc(B4OC(C)(C)C(C)(C)O4)c(N)nc3F)cc2)CC1]

To 3-bromo-6-fluoro-5-(4-(4-isopropylpiperazin-1-yl)phenyl)pyridin-2-amine (360 mg, 0.915 mmol), bis(pinacolato)diboron (256 mg, 1.007 mmol), and potassium acetate (269 mg, 2.75 mmol), was added 1,4-dioxane (10 ml). The system was degassed then [1,1′-bis(diphenylphosphino)ferrocene]dichloropalladium(II) (67.0 mg, 0.092 mmol) was added. The reaction was flushed with nitrogen and heated at 110° C. overnight. The mixture was diluted with acetonitrile, filtered through a pad of celite, and concentrated in vacuo to obtain a mixture of boronate and boronic acid which was used in the next step without further purification. LCMS: [M+H]⁺=441.38 Boronate, 357.45 Boronic acid.

Step 8: 6-(2-amino-6-fluoro-5-(4-(4-isopropylpiperazin-1-yl)phenyl)pyridin-3-yl)-4-fluoro-3-methylisoquinolin-1(2H)-one

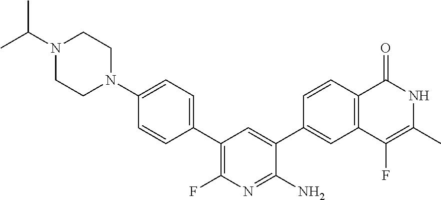 [SMILES: Cc1[nH]c(=O)c2ccc(-c3cc(-c4ccc(N5CCN(C(C)C)CC5)cc4)c(F)nc3N)cc2c1F]

Prepared from 6-bromo-4-fluoro-3-methylisoquinolin-1(2H)-one (19 mg, 0.074 mmol) and 6-fluoro-5-(4-(4-isopropylpiperazin-1-yl)phenyl)-3-(4,4,5,5-tetramethyl-1,3,2-dioxaborolan-2-yl)pyridin-2-amine (39.2 mg, 0.089 mmol) to give the title compound (3.61 mg, 9.9% yield) as a white solid. ¹H NMR (500 MHz, DMSO-d₆) δ ppm 11.23 (br. s., 1 H), 8.21 (dd, J=8.31, 1.59 Hz, 1H), 7.75 (s, 1H), 7.62-7.69 (m, 2H), 7.41 (d, J=7.82 Hz, 2H), 6.97 (d, J=8.80 Hz, 2H), 6.24 (br. s., 2H), 3.12-3.17 (m, 4H), 2.65-2.70 (m, 1H), 2.56-2.59 (m, 4H), 2.24 (d, J=2.93 Hz, 3H), 1.01 (d, J=6.48 Hz, 6H); LCMS [M+H]⁺=490.40.

### Example 369: 7-(2-amino-6-fluoro-5-(4-(4-isopropylpiperazin-1-yl)phenyl)pyridin-3-yl)-2-cyclopropylquinazolin-4(3H)-one (I-412)

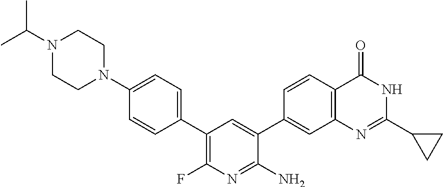 [SMILES: CC(C)N1CCN(c2ccc(-c3cc(-c4ccc5c(=O)[nH]c(C6CC6)nc5c4)c(N)nc3F)cc2)CC1]

Step 1: 7-chloro-2-cyclopropylquinazolin-4(3H)-one

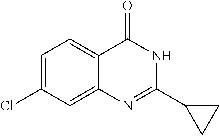 [SMILES: O=c1[nH]c(C2CC2)nc2cc(Cl)ccc12]

To a solution of 2-amino-4-chlorobenzoic acid (500 mg, 2.91 mmol), 4-(dimethylamino)pyridine (35.6 mg, 0.291 mmol), triethylamine (1.625 ml, 11.66 mmol) in anhydrous 1,2-dichloroethane (6 ml) was added dropwise cyclopropanecarbonyl chloride (0.635 ml, 6.99 mmol) at 0° C. for 20 min in an ice-water batch under nitrogen. The reaction was heated at 80° C. for 3 hours. The reaction was concentrated in vacuo to remove the volatiles to give 4-chloro-2-(cyclopropanecarboxamido)benzoic acid which was used in the next step as-is. LCMS: [M−H]⁻=238.00. 4-Chloro-2-(cyclopropanecarboxamido)benzoic acid was taken up in 27% ammonium hydroxide solution (8.41 ml, 58.3 mmol) and heated in a sealed tube at 80° C. overnight. The mixture was cooled to RT and the solid was filtered, washed with water and dried under vacuum to afford the product (535 mg, 83% yield) as a beige solid. LCMS: [M+H]⁺=221.10.

Step 2: (2-cyclopropyl-4-oxo-3,4-dihydroquinazolin-7-yl)boronic acid

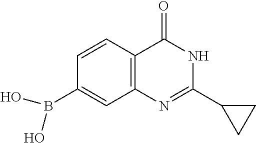 [SMILES: O=c1[nH]c(C2CC2)nc2cc(B(O)O)ccc12]

Prepared from 7-chloro-2-cyclopropylquinazolin-4(3H)-one (72 mg, 0.326 mmol) to give a mixture of boronate and boronic acid which was used in the next step without further purification. LCMS: [M+H]⁺=313.38 Boronate, 231.32 Boronic acid.

Step 3: 7-(2-amino-6-fluoro-5-(4-(4-isopropylpiperazin-1-yl)phenyl)pyridin-3-yl)-2-cyclopropylquinazolin-4(3H)-one

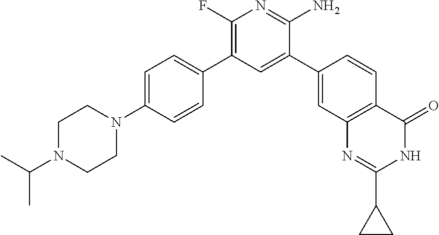 [SMILES: CC(C)N1CCN(c2ccc(-c3cc(-c4ccc5c(=O)[nH]c(C6CC6)nc5c4)c(N)nc3F)cc2)CC1]

Prepared from (2-cyclopropyl-4-oxo-3,4-dihydroquinazolin-7-yl)boronic acid (35.1 mg, 0.153 mmol) and 3-bromo-6-fluoro-5-(4-(4-isopropylpiperazin-1-yl)phenyl)pyridin-2-amine (50 mg, 0.127 mmol) to give the title compound (2.7 mg, 4.3% yield) as a white solid. ¹H NMR (500 MHz, DMSO-d₆) δ ppm 12.43 (s, 1H), 8.10 (d, J=8.19 Hz, 1H), 7.62 (d, J=10.15 Hz, 1H), 7.56 (s, 1H), 7.52 (d, J=8.19 Hz, 1H), 7.43 (br. s., 2H), 6.98 (br. s., 2H), 6.18 (br. s., 1H), 3.14 (br. s., 4H), 1.98 (d, J=4.52 Hz, 1H), 1.11 (br. s., 4H), 1.05 (d, J=7.58 Hz, 6 H); LCMS: [M+H]⁺=499.36.

### Example 370: 7-(2-amino-6-fluoro-5-(4-(4-isopropylpiperazin-1-yl)phenyl)pyridin-3-yl)-2-cyclobutylquinazolin-4(3H)-one (1-413)

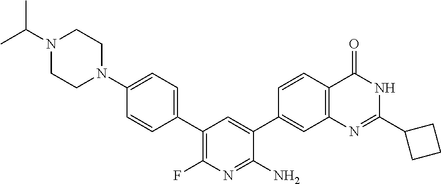 [SMILES: CC(C)N1CCN(c2ccc(-c3cc(-c4ccc5c(=O)[nH]c(C6CCC6)nc5c4)c(N)nc3F)cc2)CC1]

Step 1: 7-chloro-2-cyclobutylquinazolin-4(3H)-one

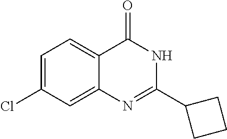 [SMILES: O=c1[nH]c(C2CCC2)nc2cc(Cl)ccc12]

To a solution of 2-amino-4-chlorobenzamide (200 mg, 1.172 mmol), triethylamine (0.487 ml, 3.50 mmol) in anhydrous dichloromethane (2.0 ml) was added dropwise cyclobutanecarbonyl chloride (0.319 ml, 2.80 mmol). The mixture was stirred at room temperature for 2 hours then heated at 40° C. for 1 hour. The reaction was concentrated in vacuo and 5% NaOH(aq) (3 ml) was added. The reaction was heated at 90° C. overnight. The reaction was cooled to RT and 1N HCl(aq) was added until ˜ pH 7. The mixture was concentrated onto celite and purified by flash chromatography (Biotage, silica gel, 0-100% EtOAc/Hexanes). The desired fractions were collected, concentrated and dried under vacuum to obtain the product (96 mg, 0.409 mmol, 35.1% yield) as a white solid. LCMS: [M+H]⁺=235.02.

Step 2: 2-cyclobutyl-7-(4,4,5,5-tetramethyl-1,3,2-dioxaborolan-2-yl)quinazolin-4(3H)-one

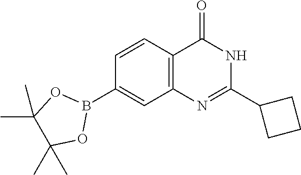 [SMILES: CC1(C)OB(c2ccc3c(=O)[nH]c(C4CCC4)nc3c2)OC1(C)C]

The title compound was prepared from 7-chloro-2-cyclobutylquinazolin-4(3H)-one (54 mg, 0.230 mmol) to give a mixture of boronate and boronic acid which was used in the next step without further purification. LCMS [M+H]⁺ 327.43 Boronate, 245.24 Boronic acid.

Step 3: 7-(2-amino-6-fluoro-5-(4-(4-isopropylpiperazin-1-yl)phenyl)pyridin-3-yl)-2-cyclobutylquinazolin-4(3H)-one

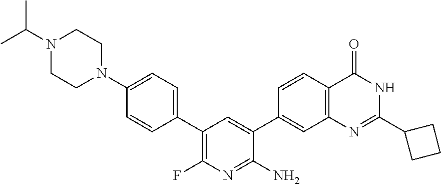 [SMILES: CC(C)N1CCN(c2ccc(-c3cc(-c4ccc5c(=O)[nH]c(C6CCC6)nc5c4)c(N)nc3F)cc2)CC1]

Prepared from 2-cyclobutyl-7-(4,4,5,5-tetramethyl-1,3,2-dioxaborolan-2-yl)quinazolin-4(3H)-one (60.7 mg, 0.186 mmol) and 3-bromo-6-fluoro-5-(4-(4-isopropylpiperazin-1-yl)phenyl)pyridin-2-amine (61 mg, 0.155 mmol) to give the title compound (12.47 mg, 0.024 mmol, 15.68%) as a beige solid. ¹H NMR (500 MHz, DMSO-d₆) δ ppm 12.06 (s, 1H), 8.12 (d, J=8.07 Hz, 1H), 7.73 (s, 1H), 7.65 (d, J=10.15 Hz, 1H), 7.58 (dd, J=8.07, 1.22 Hz, 1H), 7.43 (d, J=8.19 Hz, 2H), 6.97 (d, J=8.56 Hz, 2H), 6.21 (s, 2H), 3.52 (quin, J=8.56 Hz, 1H), 3.15 (br. s., 4H), 2.68 (br. s., 1H), 2.59 (br. s., 4H), 2.38-2.47 (m, 2H), 2.22-2.31 (m, 2H), 1.95-2.04 (m, 1H), 1.85 (q, J=9.62 Hz, 1H), 1.02 (d, J=4.28 Hz, 6H); LCMS [M+H]⁺=513.40.

### Example 371: 7-(2-amino-6-fluoro-5-(4-(4-isopropylpiperazin-1-yl)phenyl)pyridin-3-yl)-2-methoxyquinazolin-4(3H)-one (I-419)

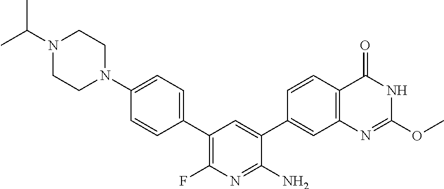 [SMILES: COc1nc2cc(-c3cc(-c4ccc(N5CCN(C(C)C)CC5)cc4)c(F)nc3N)ccc2c(=O)[nH]1]

Step 1: 7-bromo-2-methoxyquinazolin-4(3H)-one

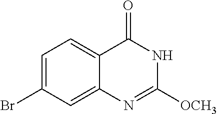 [SMILES: COc1nc2cc(Br)ccc2c(=O)[nH]1]

To a solution of 7-bromo-2-chloroquinazolin-4(3H)-one (100 mg, 0.385 mmol) in tetrahydrofuran (1.0 ml) was added sodium methoxide, 0.5 M in MeOH (1.2 ml, 0.578 mmol) and heated at 90° C. for 5 days. The reaction was quenched with saturated aqueous NH₄Cl solution and then concentrated onto celite. The crude mixture was purified by flash chromatography (Biotage, silica gel) eluting with 0-100% EtOAc/Hexanes. The desired fractions were collected, concentrated and dried under vacuum to afford the product (12 mg, 12% yield) as a white solid. LCMS [M+H]⁺ 255.08.

Step 2: 7-(2-amino-6-fluoro-5-(4-(4-isopropylpiperazin-1-yl)phenyl)pyridin-3-yl)-2-methoxyquinazolin-4(3H)-one

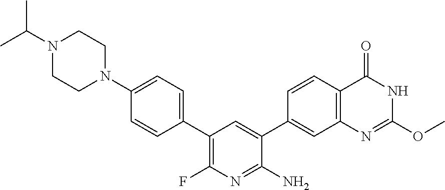 [SMILES: COc1nc2cc(-c3cc(-c4ccc(N5CCN(C(C)C)CC5)cc4)c(F)nc3N)ccc2c(=O)[nH]1]

The title compound was prepared from 7-bromo-2-methoxyquinazolin-4(3H)-one (12 mg, 0.047 mmol) and 6-fluoro-5-(4-(4-isopropylpiperazin-1-yl)phenyl)-3-(4,4,5,5-tetramethyl-1,3,2-dioxaborolan-2-yl)pyridin-2-amine (24.86 mg, 0.056 mmol) to give the title compound (2.2 mg, 9.6% yield) as a white solid. 1 H NMR (500 MHz, DMSO-d6) δ ppm 7.98 (d, J=8.07 Hz, 1H), 7.59 (d, J=10.15 Hz, 1H), 7.42 (br. s., 1H), 7.40 (s, 2H), 7.26 (br. s., 1 H), 6.96 (d, J=8.80 Hz, 2H), 6.08 (br. s., 2H), 3.84 (s, 3H), 3.10-3.19 (m, 4H), 2.68 (d, J=6.48 Hz, 1H), 2.56-2.59 (m, 4H), 1.01 (d, J=6.60 Hz, 6H); LCMS: [M+H]⁺=489.39.

### Example 372: 2-amino-7-(2-amino-6-fluoro-5-(4-(4-isopropylpiperazin-1-yl)phenyl)pyridin-3-yl)quinazolin-4(3H)-one (I-422)

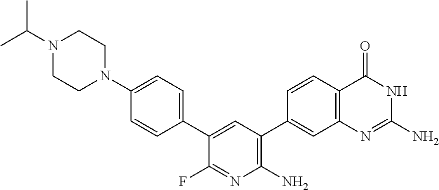 [SMILES: CC(C)N1CCN(c2ccc(-c3cc(-c4ccc5c(=O)[nH]c(N)nc5c4)c(N)nc3F)cc2)CC1]

Prepared from 2-amino-7-bromo-3H-quinazolin-4-one (35 mg, 0.146 mmol) and 6-fluoro-5-(4-(4-isopropylpiperazin-1-yl)phenyl)-3-(4,4,5,5-tetramethyl-1,3,2-dioxaborolan-2-yl)pyridin-2-amine (77 mg, 0.175 mmol) to give the title compound (18.4 mg, 26.7%) as an off-white solid. ¹H NMR (500 MHz, DMSO-d₆) δ ppm 11.35 (br. s., 1H), 7.93 (d, J=8.07 Hz, 1H), 7.58 (d, J=10.15 Hz, 1H), 7.40 (d, J=8.07 Hz, 2H), 7.25 (s, 1H), 7.17 (d, J=8.07 Hz, 1H), 6.96 (d, J=8.80 Hz, 2H), 6.56 (br. s., 2H), 6.11 (br. s., 2H), 3.12-3.17 (m, 4H), 2.64-2.70 (m, 1H), 2.55-2.60 (m, 4H), 1.01 (d, J=6.48 Hz, 6H); LCMS: [M+H]⁺=474.41.

### Example 373: 6-(2-amino-5-(3-((dimethylamino)methyl)-4-(tetrahydro-2H-pyran-4-yl)phenyl)-6-fluoropyridin-3-yl)-4-methylisoquinolin-1(2H)-one (I-426)

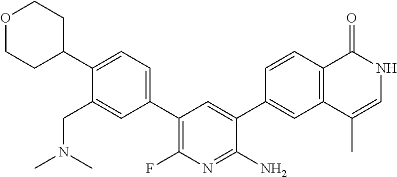 [SMILES: Cc1c[nH]c(=O)c2ccc(-c3cc(-c4ccc(C5CCOCC5)c(CN(C)C)c4)c(F)nc3N)cc12]

Step 1 (E)-3-(3-bromophenyl)but-2-enoic acid

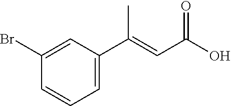

To THF (150 mL) at 0° C. under nitrogen atmosphere, sodium hydride (60%, 6 g, 150.71 mmol) was add portion wise. To this, triethyl phosphonoacetate (21.47 mL, 113.06 mmol) was added dropwise and stirred at 0° C. for 10 min. The resulting reaction mass was added dropwise to a solution of 1-(3-bromophenyl)ethan-1-one (15 g, 75.35 mmol) in THF (150 mL) and the reaction mass was reflux for 16 h. After completion of the reaction, reaction mass was diluted with water (500 mL) and extracted with DCM (3×500 mL). The organic layer was dried over anhydrous Na₂SO₄ and concentrated under vacuum to afford a yellow oil. The oil was dissolved in methanol (150 mL) and sodium hydroxide (9.04 g, 226.13 mmol) and water (75 mL) were added and the reaction mass was heated at 50° C. for 2 h. After completion of the reaction, the reaction mass was concentrated to evaporate the organics. The aqueous solution was acidified by aqueous 2M HCl and extracted with ethyl acetate (2×500 mL). The combined organic layer was dried over anhydrous sodium sulfate and concentrated under vacuum to afford the product (7 g, 39%) as yellow solid LCMS: [M−2H]⁺=238.9.

Step 2: (E)-3-(3-bromophenyl)but-2-enoyl chloride

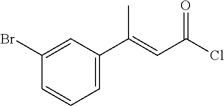

To a cooled solution of (E)-3-(3-bromophenyl)but-2-enoic acid (6 g, 24.88 mmol) in DCM (60 mL) and DMF (0.2 mL) at 0° C., oxaloyl chloride (2.59 mL, 29.86 mmol) was added dropwise and the reaction mass was allowed to warm to RT and stirred at same temperature for 3 h. After completion the reaction, reaction mass was concentrated and azeotrope with toluene (2×20 mL) and DCM (2×20 mL) to get the crude product which was used as is in the next step.

Step 3: (E)-3-(3-bromophenyl)but-2-enoyl azide

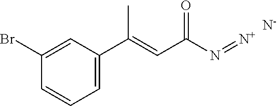

To a cooled solution of crude of (E)-3-(3-bromophenyl)but-2-enoyl chloride (6 g, 23.11 mmol) in 1,4 dioxane (60 mL) at 0° C., a suspension of sodium azide (2.26 g, 34.47 mmol) in 1:1 mixture of 1,4-dioxane and water (20 mL) was added and the reaction mas was gradually warm to room temperature and stirred at same temperature for a 3 h. After completion reaction, reaction mass was diluted with water (70 mL) and extracted with diethyl ether (2×200 mL). The combined organic mixture was back washed with saturated sodium bicarbonate solution (3×200 mL) and water (3×200 mL) and dried over anhydrous Na₂SO₄ and organic layer was directly used for next step.

Step 4: 6-bromo-4-methylisoquinolin-1(2H)-one

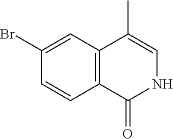

To the ether layer of (E)-3-(3-bromophenyl)but-2-enoyl azide is treated with 1,2 dichlorobenzene (15 mL) and the ether was removed under vacuum to give a solution of (E)-3-(3-bromophenyl)but-2-enoyl azide in 1,2 dichlorobenzene. The acyl azide solution in 1,2 dichlorobenzene was added dropwise over 30 min to a solution of iodine (3 crystal) in 1,2 dichlorobenzene (15 mL) at 120° C. After completion of addition, reaction mass was stirred at 190° C. for 16 h. After completion of the reaction, reaction mass was allowed to cool room temperature and added to hexane (800 mL). The suspension stirred for 1 h and the solid thus obtained was filtration, washed with ethyl acetate (20 mL) and DCM (20 mL) and dried under vacuum to give the product (1.25 g, 23%) as pale yellow solid. LCMS: [M+2]⁺=240.8

Step 5: 4-methyl-6-(4,4,5,5-tetramethyl-1,3,2-dioxaborolan-2-yl)isoquinolin-1(2H)-one

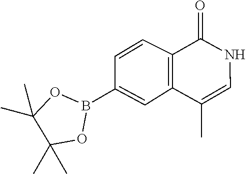 [SMILES: Cc1c[nH]c(=O)c2ccc(B3OC(C)(C)C(C)(C)O3)cc12]

To 6-bromo-4-methylisoquinolin-1(2H)-one (742 mg, 3.12 mmol), bis(pinacolato)diboron (871 mg, 3.43 mmol), and potassium acetate (918 mg, 9.35 mmol), was added anhydrous 1,4-dioxane (20 ml). The system was flushed with nitrogen then [1,12-bis(diphenylphosphino)ferrocene]dichloropalladium(II) (228 mg, 0.312 mmol) was added. The mixture was flushed with nitrogen then heated at 100° C. for 2 hours. The reaction was diluted with acetonitrile, filtered through a pad of celite, concentrated in vacuo and used crude in the next step (120 mg, 0.341 mmol, 100%) as a mixture of boronate and boronic acid. LCMS: [M+H]⁺=285.99 Boronate, 204.29 Boronic acid

Step 6: 6-(2-amino-5-bromo-6-fluoropyridin-3-yl)-4-methylisoquinolin-1(2H)-one

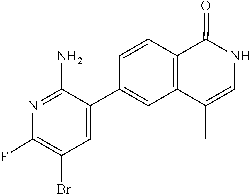 [SMILES: Cc1c[nH]c(=O)c2ccc(-c3cc(Br)c(F)nc3N)cc12]

To 5-bromo-6-fluoro-3-iodopyridin-2-amine (405 mg, 1.278 mmol) and 4-methyl-6-(4,4,5,5-tetramethyl-1,3,2-dioxaborolan-2-yl)isoquinolin-1(2H)-one (437 mg, 1.534 mmol) in acetonitrile (10 ml) was added a solution of sodium carbonate monohydrate (333 mg, 2.68 mmol) in water (2.5 mL). The system was degassed and bis(triphenylphosphine)palladium(II) dichloride (108 mg, 0.153 mmol) was added. The reaction was flushed with nitrogen then heated at 90° C. overnight. The reaction was concentrated onto celite and purified by flash chromatography (Biotage, silica gel) eluting with 0-100% EtOAc/Hexanes. The desired fractions were collected, concentrated, triturated from ether and dried under vacuum to afford 6-(2-amino-5-bromo-6-fluoropyridin-3-yl)-4-methylisoquinolin-1(2H)-one (178 mg, 0.363 mmol, 28.4% yield) as a grey solid. LCMS [M +H]⁺=348.18.

Step 7: Methyl 2-(3,6-dihydro-2H-pyran-4-yl)-5-nitrobenzoate

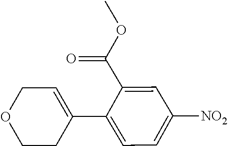 [SMILES: COC(=O)c1cc([N+](=O)[O-])ccc1C1=CCOCC1]

To a degassed solution of methyl 2-iodo-5-nitrobenzoate (4 g, 13.02 mmol), 2-(3,6-dihydro-2H-pyran-4-yl)-4,4,5,5-tetramethyl-1,3,2-dioxaborolane (3.4 g, 13.15 mmol) and K₂CO₃ (4.5 g, 32.56 mmol) in dioxane: water (7.5: 2.5, 40 mL), Pd(dppf)Cl2.DCM (1.06 g, 1.3 mmol) was added and further degassed with N2 for 10 minutes and the reaction mass was stirred at 90° C. for 16 h. After completion of reaction, reaction mass was diluted with water (40 mL) and extracted with EtOAc (3×30 mL). The crude was purified by column chromatography and the product was eluted in 18% EtOAc in hexanes to afford the product (3.1 g, 90%) as off-white solid. ¹H NMR (400 MHz, Chloroform-d) δ 8.74 (d, J=2.4 Hz, 1H), 8.47 (s, 1H), 8.35 (dd, J=8.4, 2.5 Hz, 1H), 7.46 (d, J=8.4 Hz, 1H), 5.7 (s, 1H), 4.35 (q, J=2.7 Hz, 2H), 3.98 (d, J=6.6 Hz, 5H), 2.41 (tq, J=4.9, 2.3 Hz, 2H).

Step 8: Methyl 5-amino-2-(tetrahydro-2H-pyran-4-yl)benzoate

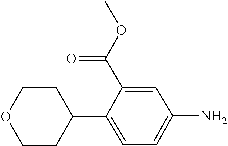 [SMILES: COC(=O)c1cc(N)ccc1C1CCOCC1]

In a 3 neck RBF, 10% Pd/C (50% moist, 2w/w, 7.6 g) and methyl 2-(3,6-dihydro-2H-pyran-4-yl)-5-nitrobenzoate (3.8 g, 14.43 mmol) were added in EtOAc (40 mL). The reaction mass was stirred at room temperature with continuous purging of hydrogen for 2.5 days. After completion of reaction, reaction mass was filtered through celite bed and the filtrate was concentrated under vacuum to afford the crude product (3.2 g, 94%) as pale yellow gummy solid used as such in next step without further purification. LCMS [M+H]⁺=236.5.

Step 9: Methyl 5-bromo-2-(tetrahydro-2H-pyran-4-yl)benzoate

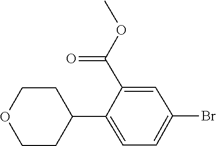 [SMILES: COC(=O)c1cc(Br)ccc1C1CCOCC1]

To a solution of methyl 5-amino-2-(tetrahydro-2H-pyran-4-yl)benzoate (3 g, 12.74 mmol) in bromoform (9.6 mL). To this, tert-butylnitrite (15 mL) was added dropwise at room temperature and the reaction mass was stirred at same temperature for 30 minutes. After completion of reaction, the reaction mass was concentrated to afford crude. The crude was purified by column chromatography and the product was eluted in 10% EtOAc in hexanes to afford the product (1.8 g, 47%) as a yellow solid. ¹H NMR (400 MHz, Chloroform-d) 1.76-1.90 (m, 4H), 3.63 (m, 4H), 3.95 (s, 3H), 4.11 (dt, J=11.1, 2.9 Hz, 1H), 7.32 (d, J=8.4 Hz, 1H), 7.64 (dd, J=8.4, 2.3 Hz, 1H), 7.98 (d, J=2.4 Hz, 1H).

Step 10: (5-bromo-2-(tetrahydro-2H-pyran-4-yl)phenyl)methanol

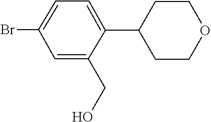 [SMILES: OCc1cc(Br)ccc1C1CCOCC1]

To a cooled solution of methyl 5-bromo-2-(tetrahydro-2H-pyran-4-yl)benzoate (1.8 g, 6.02 mmol) in dry THF (18 mL) at 0° C., LAH (1 M in THF, 6.12 mL, 6.02 mmol) was added dropwise at 0° C. The reaction mass was stirred at RT for 3 h. After completion of reaction, reaction mass was quenched with the dil. HCl (50 mL) and extracted with ethyl acetate (3×50 mL). The combined organic was dried over anhydrous Na₂SO₄, concentrate under vacuum to afford the title compound (1.3 g, 4.79 mmol, 79.68%) as pale-yellow liquid. ¹H NMR (400 MHz, Chloroform-d) 1.67-1.90 (m, 4H), 3.05 (m, 1H), 3.58 (m, 2H), 4.10 (d, J=11.1, 1H), 4.73 (s, 2H), 7.21 (d, J=8.4 Hz, 1H), 7.47 (dd, J=8.4, 2.3 Hz, 1H), 7.55 (d, J=2.4 Hz, 1H).

Step 11: 5-bromo-2-(tetrahydro-2H-pyran-4-yl)benzaldehyde

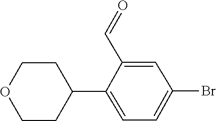 [SMILES: O=Cc1cc(Br)ccc1C1CCOCC1]

To a solution of (5-bromo-2-(tetrahydro-2H-pyran-4-yl)phenyl)methanol (1.3 g, 4.79 mmol) in DCM (26 mL), MnO₂ (3.3 g, 38.35 mmol) was added at room temperature and the reaction mass was stirred at room temperature for 48 h. After completion of reaction, reaction mass was filtered through celite bed and the filtrate was concentrate under vacuum to afford crude product. The crude was purified by column chromatography and the product was eluted in 15% EtOAc in hexanes to afford the product (0.76 g, 58.9%) as light-yellow solid. ¹H NMR (400 MHz, Chloroform-d) δ 1.76-1.84 (m, 2H), 1.86-1.93 (m, J=12.2, 4.3 Hz, 2H), 3.61-3.67 (t, J=11.7 Hz, 2H), 3.89-3.83 (tt, J=3.2, 4.0 Hz, 1H), 4.15-4.12 (dd, J=11.4, 4.6 Hz, 2H), 7.39-7.37 (t, J=8.4 Hz, 1H), 7.77 (d, 1H), 7.98 (s, 1H), 10.27 (s, 1H).

Step 12: 1-(5-bromo-2-(tetrahydro-2H-pyran-4-yl)phenyl)-N,N-dimethylmethanamine

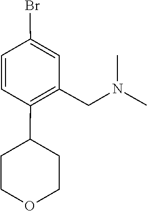 [SMILES: CN(C)Cc1cc(Br)ccc1C1CCOCC1]

To a solution of 5-bromo-2-(tetrahydro-2H-pyran-4-yl)benzaldehyde (400 mg, 1.486 mmol) in dichloromethane (DCM) (10 ml) was added dimethylamine, 2.0M solution in THF (2.229 ml, 4.46 mmol) followed by acetic acid, glacial, 99.8% (0.00850 ml, 0.149 mmol). Upon stirring at room temperature for 10 minutes, sodium triacetoxyborohydride (945 mg, 4.46 mmol) was added portion-wise and the white suspension was stirred at room temperature for 1 hour. The reaction was basified with 1M NaOH(aq) solution. The organic phase was separated, and the aqueous phase was further washed with DCM (2x). The combined organic phases were washed with brine (1x), dried over anhydrous sodium sulfate and concentrated in vacuo to obtain 1-(5-bromo-2-(tetrahydro-2H-pyran-4-yl)phenyl)-N,N-dimethylmethanamine (394 mg, 1.321 mmol, 89% yield) as a white solid. The material was carried onto the next step without further purification. LCMS [M+H]⁺298.34.

Step 13: N,N-dimethyl-1-(2-(tetrahydro-2H-pyran-4-yl)-5-(4,4,5,5-tetramethyl-1,3,2-dioxaborolan-2-yl)phenyl)methanamine

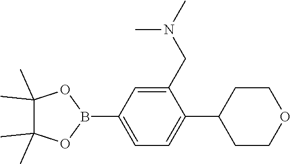 [SMILES: CN(C)Cc1cc(B2OC(C)(C)C(C)(C)O2)ccc1C1CCOCC1]

To a vial charged with 1-(5-bromo-2-(tetrahydro-2H-pyran-4-yl)phenyl)-N,N-dimethylmethanamine (385 mg, 1.291 mmol), bis(pinacolato)diboron (361 mg, 1.420 mmol), and potassium acetate (380 mg, 3.87 mmol), was added anhydrous 1,4-dioxane (5 ml). The system was degassed then [1,12-bis(diphenylphosphino)ferrocene] dichloropalladium(II) (94 mg, 0.129 mmol) was added. The reaction was flushed with nitrogen and heated at 100° C. overnight. The mixture was diluted with acetonitrile, filtered through a pad of celite, concentrated in vacuo and used as-is the next step assuming full conversion to the boronate. LCMS [M+H]⁺=346.55.

Step 14: 6-(2-amino-5-(3-((dimethylamino)methyl)-4-(tetrahydro-2H-pyran-4-yl)phenyl)-6-fluoropyridin-3-yl)-4-methylisoquinolin-1(2H)-one

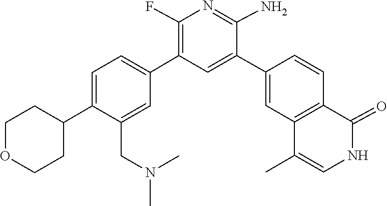 [SMILES: Cc1c[nH]c(=O)c2ccc(-c3cc(-c4ccc(C5CCOCC5)c(CN(C)C)c4)c(F)nc3N)cc12]

To 6-(2-amino-5-bromo-6-fluoropyridin-3-yl)-4-methylisoquinolin-1(2H)-one (35 mg, 0.101 mmol), N,N-dimethyl-1-(2-(tetrahydro-2H-pyran-4-yl)-5-(4,4,5,5-tetramethyl-1,3,2-dioxaborolan-2-yl)phenyl)methanamine (41.7 mg, 0.121 mmol), Cs₂CO₃ (98 mg, 0.302 mmol) in a mixture of 1,4-dioxane (5 ml): water (1 ml) was added tetrakis(triphenylphosphine)palladium(0) (13.94 mg, 0.012 mmol). The reaction was flushed with nitrogen and heated at 90° C. for 3 hours. The mixture was concentrated onto celite and purified by flash chromatography to afford the title compound (25.6 mg, 52.3% yield) as a grey solid. ¹H NMR (500 MHz, DMSO-d₆) δ ppm 11.10 (d, J=5.01 Hz, 1H), 8.28 (d, J=8.19 Hz, 1H), 7.72 (d, J=0.98 Hz, 1H), 7.70 (d, J=10.03 Hz, 1H), 7.62 (dd, J=8.31, 1.34 Hz, 1 H), 7.44 (d, J=8.19 Hz, 1H), 7.39 (s, 1H), 7.34 (d, J=8.07 Hz, 1H), 7.04 (d, J=4.52 Hz, 1H), 6.34 (s, 2H), 3.95 (dd, J=10.88, 3.42 Hz, 2H), 3.41-3.47 (m, 4H), 3.16-3.23 (m, 1H), 2.24 (s, 3H), 2.16 (s, 6H), 1.70 (qd, J=12.25, 4.10 Hz, 2H), 1.58-1.65 (m, 2H); LCMS [M+H]⁺=487.45.

### Example 368: 6-(6′-amino-6-((dimethylamino)methyl)-2′-fluoro-5-morpholino-[2,3-bipyridin]-5′-yl)-7-fluoro-3,4-dihydroisaoquinolin-1(2H)-one (I-427)

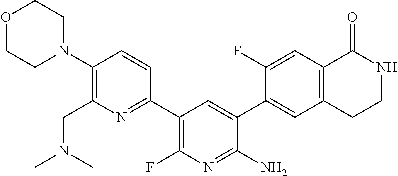 [SMILES: CN(C)Cc1nc(-c2cc(-c3cc4c(cc3F)C(=O)NCC4)c(N)nc2F)ccc1N1CCOCC1]

Step 1: Preparation of 6-bromo-3-morpholinopicolinaldehyde

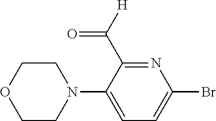 [SMILES: O=Cc1nc(Br)ccc1N1CCOCC1]

Prepared by reacting 2-bromo-5-fluoro-6-formylpyridine (2.5 g, 12.25 mmol) and morpholine (1.269 mL, 14.71 mmol) at 85° C. for 6 h. The product was isolated as a pale-yellow oil (2.75 g, 79% yield). LCMS: [M+H]⁺=271.1.

Step 2: 1-(6-bromo-3-morpholinopyridin-2-yl)-N,N-dimethylmethanamine

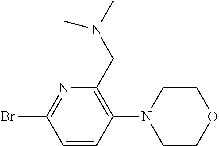 [SMILES: CN(C)Cc1nc(Br)ccc1N1CCOCC1]

Prepared using 6-bromo-3-morpholinopicolinaldehyde (500 mg, 1.752 mmol) and dimethylamine, 2.0M solution in THF (0.876 mL, 1.752 mmol). Silica gel chromatography afforded the product as a cream colored solid (336 mg, 62% yield). LCMS: [M+H]⁺=300.35.

Step 3: 6-(6′-amino-6-((dimethylamino)methyl)-2′-fluoro-5-morpholino-[2,3′-bipyridin]-5′-yl)-7-fluoro-3,4-dihydroisoquinolin-1(2H)-one

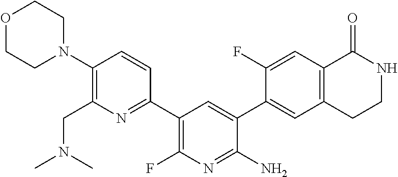 [SMILES: CN(C)Cc1nc(-c2cc(-c3cc4c(cc3F)C(=O)NCC4)c(N)nc2F)ccc1N1CCOCC1]

Prepared as per previous procedures from the intermediate 1-(6-bromo-3-morpholinopyridin-2-yl)-N,N-dimethylmethanamine (50 mg, 0.162 mmol) from with 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (85 mg, 0.211 mmol) to yield the title compound as a white solid (48 mg, 57% yield). ¹H NMR (500 MHz, DMSO-d₆) δ=8.14-8.11 (m, 1H), 8.09-8.01 (m, 1H), 7.68-7.64 (m, 1H), 7.64-7.61 (m, 1H), 7.58-7.51 (m, 1H), 7.43-7.40 (m, 1H), 6.57-6.37 (m, 2H), 3.80-3.73 (m, 4H), 3.53 (br s, 2H), 3.45-3.40 (m, 2H), 3.05 (br s, 4H), 2.96-2.91 (m, 2H), 2.24 (s, 6H). LCMS: [M+H]⁺=495.54

### Example 369: 6-(2-amino-5-(3-((ethyl(methyl)amino)methyl)-4-(tetrahydro-2H-pyran-4-yl)phenyl)-6-fluoropyridin-3-yl)-3,4-dihydroisoquinolin-1(2H)-one (I-428)

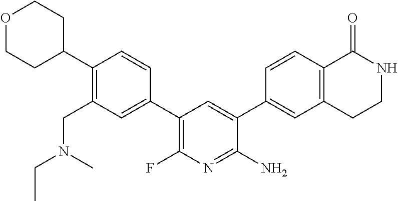 [SMILES: CCN(C)Cc1cc(-c2cc(-c3ccc4c(c3)CCNC4=O)c(N)nc2F)ccc1C1CCOCC1]

Step 1: Preparation of 1-(5-bromo-2-(tetrahydro-2H-pyran-4-yl)benzyl)-4-methoxypiperidine

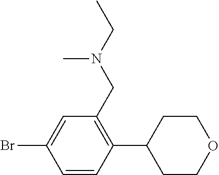 [SMILES: CCN(C)Cc1cc(Br)ccc1C1CCOCC1]

Prepared by a procedure analogous the previous example using 5-bromo-2-(tetrahydro-2H-pyran-4-yl)benzaldehyde (400 mg, 1.486 mmol) and N-ethylmethylamine (0.511 ml, 5.94 mmol) as the amine. The product was isolated as a white solid (444 mg, 91 % yield). LCMS: [M+H]⁺=312.38.

Step 2: Preparation of 6-(2-amino-5-(3-((ethyl(methyl)amino)methyl)-4-(tetrahydro-2H-pyran-4-yl)phenyl)-6-fluoropyridin-3-yl)-3,4-dihydroisoquinolin-1(2H)-one

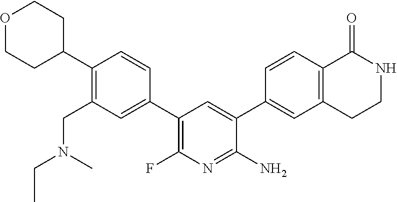 [SMILES: CCN(C)Cc1cc(-c2cc(-c3ccc4c(c3)CCNC4=O)c(N)nc2F)ccc1C1CCOCC1]

Prepared by coupling the intermediate N-(5-bromo-2-(tetrahydro-2H-pyran-4-yl)benzyl)-N-methylethanamine (55 mg, 0.167 mmol) with 6-(2-amino-6-fluoro-5-(4,4,5,5-tetramethyl-1,3,2-dioxaborolan-2-yl)pyridin-3-yl)-3,4-dihydroisoquinolin-1(2H)-one (83 mg, 0.218 mmol) to yield the title compound as a white solid (65 mg, 76% yield). ¹H NMR (500 MHz, DMSO-d₆) δ=8.00-7.93 (m, 1H), 7.93-7.88 (m, 1H), 7.66-7.60 (m, 1H), 7.49-7.46 (m, 1H), 7.46-7.41 (m, 2H), 7.41-7.37 (m, 1H), 7.37-7.32 (m, 1H), 6.37-6.16 (m, 2H), 4.02-3.94 (m, 2H), 3.54-3.48 (m, 2H), 3.46-3.39 (m, 4H), 3.27-3.21 (m, 1H), 2.99-2.93 (m, 2H), 2.43-2.37 (m, 2H), 2.13-2.05 (m, 3H), 1.75-1.66 (m, 2H), 1.66-1.59 (m, 2H), 1.09-1.02 (m, 3H). LCMS: [M+H]+=489.52Example 371: 6-(2-amino-5-(3-((dimethylamino)methyl)-4-(tetrahydro-2H-pyran-4-yl)phenyl)-6-fluoropyridin-3-yl)-7-fluoro-3,4-dihydroisoquinolin-1(2H)-one (I-429)

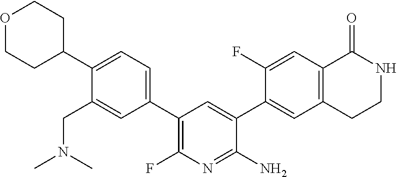 [SMILES: CN(C)Cc1cc(-c2cc(-c3cc4c(cc3F)C(=O)NCC4)c(N)nc2F)ccc1C1CCOCC1]

Prepared from 1-(5-bromo-2-(tetrahydro-2H-pyran-4-yl)phenyl)-N,N-dimethylmethanamine (60 mg, 0.157 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (63 mg, 0.157 mmol) to afford the title compound as a white solid (24 mg, 30% yield). ¹H NMR (500 MHz, METHANOL-d₄) δ ppm 7.63 (d, J=10.03 Hz, 1H), 7.54-7.57 (m, 1H), 7.33-7.36 (m, 1H), 7.28-7.32 (m, 2H), 7.26 (d, J=8.19 Hz, 1H), 3.94 (dd, J=11.19, 3.73 Hz, 2H), 3.41-3.52 (m, 6H), 3.10-3.17 (m, 1H), 2.91 (t, J=6.60 Hz, 2H), 2.12-2.21 (m, 6H), 1.68-1.80 (m, 2H), 1.60 (d, J=12.96 Hz, 2H). LCMS: [M+H]⁺=493.34.

### Example 372: 6-(2-amino-6-fluoro-5-(4-(2-methyl-2,7-diazaspiro[3.5]nonan-7-yl)phenyl)pyridin-3-yl)-3,4-dihydroisoquinolin-1(2H)-one (I-430)

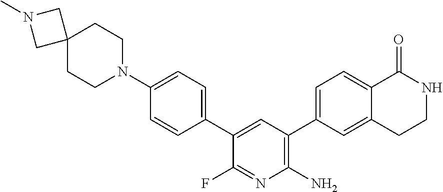 [SMILES: CN1CC2(CCN(c3ccc(-c4cc(-c5ccc6c(c5)CCNC6=O)c(N)nc4F)cc3)CC2)C1]

Step 1: tert-Butyl 7-(4-(4,4,5,5-tetramethyl-1,3,2-dioxaborolan-2-yl)phenyl)-2,7-diazaspiro[3.5]nonane-2-carboxylate

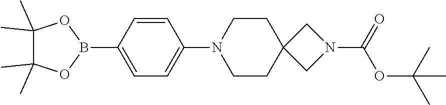 [SMILES: CC(C)(C)OC(=O)N1CC2(CCN(c3ccc(B4OC(C)(C)C(C)(C)O4)cc3)CC2)C1]

4-Bromophenylboronic acid, pinacol ester (813 mg, 2.87 mmol), sodium tert-butoxide(318 g, 3.31 mmol) tert-butyl 2,7-diazaspiro[3.5]nonane-2-carboxylate (500 mg, 2.21 mmol), 2-dicyclohexylphosphino-2′,6′-di-i-propoxy-1,1′-biphenyl (10.3 mg, 0.022 mmol) and chloro-(2-Dicyclohexylphosphino-2′,6′-diisopropoxy-1,1′-biphenyl)[2-(2-aminoethyl)phenyl]palladium(II)—methyl-t-butyl ether adduct (18.1 mg, 0.0.22 mmol) were mixed together in 1,4-Dioxane (8 mL) under vacuum. The reaction mixture was stirred at r.t for 2 days. The reaction mixture was diluted with EtOAc, washed with Brine, concentrated with silica gel and purified by column chromatography, eluted with EtOAc to give the product (572 mg, 60.4% yield) as off-white solid. LCMS: [M+H]⁺=429.29.

Step 2: 7-(4-(4,4,5,5-tetramethyl-1,3,2-dioxaborolan-2-yl)phenyl)-2,7-diazaspiro[3.5]nonane dihydrochloride

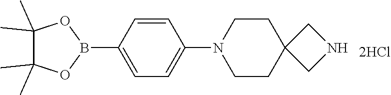 [SMILES: CC1(C)OB(c2ccc(N3CCC4(CC3)CNC4)cc2)OC1(C)C]

tert-Butyl 7-(4-(4,4,5,5-tetramethyl-1,3,2-dioxaborolan-2-yl)phenyl)-2,7-diazaspiro[3.5]nonane-2-carboxylate (543 mg, 1.268 mmol) was mixed with ether (5 mL) and MeOH (5 mL) at r.t., then Hydrogen chloride solution 4.0 M in dioxane (3.17 mL, 12.68 mmol) were added. The reaction mixture was stirred at r.t. overnight. The reaction mixture was concentrated by rotavapor. The residue was triturated with EtOAc to give the product (500 mg, 98% yield) as white solid. LCMS: [M+H]⁺=329.43.
Step 3: 2-methyl-7-(4-(4,4,5,5-tetramethyl-1,3,2-dioxaborolan-2-yl)phenyl)-2,7-diazaspiro[3.5]nonane

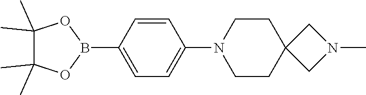

Prepared from 7-(4-(4,4,5,5-tetramethyl-1,3,2-dioxaborolan-2-yl)phenyl)-2,7-diazaspiro[3.5]nonane dihydrochloride (180 mg, 0.449 mmol) reacted with Formaldehyde solution, 37% wt in water (54.6 mg, 0.673 mmol) and Sodium triacetoxyborohydride (143 mg, 0.673 mmol), sodium acetate (73.6 mg, 0.897 mmol) in DCM (2.5 mL) and MeOH (0.5 mL) at r.t. for an hour to give after standard workup the product (143 mg, 93% yield). LCMS: [M+H]⁺=343.41;

Step 4 6-(2-amino-6-fluoro-5-(4-(2-methyl-2,7-diazaspiro[3.5]nonan-7-yl)phenyl)pyridin-3-yl)-3,4-dihydroisoquinolin-1(2H)-one

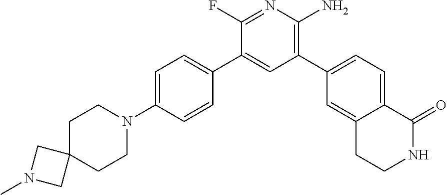 [SMILES: CN1CC2(CCN(c3ccc(-c4cc(-c5ccc6c(c5)CCNC6=O)c(N)nc4F)cc3)CC2)C1]

Prepared from 6-(2-amino-6-fluoro-5-(4-(2-methyl-2,7-diazaspiro[3.5]nonan-7-yl)phenyl)pyridin-3-yl)-3,4-dihydroisoquinolin-1(2H)-one was prepared from 6-(2-amino-5-bromo-6-fluoropyridin-3-yl)-3,4-dihydroisoquinolin-1(2H)-one (40 mg, 0.119 mmol) and 2-methyl-7-(4-(4,4,5,5-tetramethyl-1,3,2-dioxaborolan-2-yl)phenyl)-2,7-diazaspiro[3.5]nonane (33.9 mg, 0.099 mmol), to give the title compound (29.5 mg, 60% yield). ¹H NMR (500 MHz, DMSO-d₆) δ=7.93 (br s, 1H), 7.90 (d, J=7.8 Hz, 1H), 7.57 (d, J=10.1 Hz, 1H), 7.48-7.42 (m, 2H), 7.38 (br d, J=7.9 Hz, 2H), 6.96 (d, J=8.8 Hz, 2H), 6.16 (s, 2H), 3.44-3.38 (m, 2H), 3.15-3.08 (m, 4H), 2.99-2.89 (m, 6H), 2.22 (s, 3H), 1.78-1.72 (m, 4H); LCMS: [M+H]⁺=472.41.

### Example 373: 6-(2-amino-6-fluoro-5-(5-methyl-1,2,3,4-tetrahydroisoquinolin-7-yl)pyridin-3-yl)-3,4-dihydroisoquinolin-1(2H)-one (I-431)

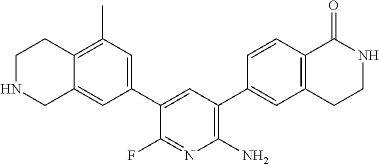 [SMILES: Cc1cc(-c2cc(-c3ccc4c(c3)CCNC4=O)c(N)nc2F)cc2c1CCNC2]

Prepared from 7-bromo-5-methyl-1,2,3,4-tetrahydroisoquinoline (40 mg, 0.177 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (85 mg, 0.221 mmol) to yield the title compound as a beige solid (65 mg, 87% yield). ¹H NMR (500 MHz, METHANOL-d₄) δ ppm 8.05 (d, J=7.95 Hz, 1H), 7.62 (d, J=9.66 Hz, 1H), 7.51 (d, J=7.95 Hz, 1H), 7.46 (s, 1H), 7.20 (s, 1H), 7.07 (s, 1H), 3.99 (s, 2H), 3.56 (t, J=6.66 Hz, 2H), 3.16 (t, J=6.05 Hz, 2H), 3.02-3.10 (m, 2H), 2.67-2.80 (m, 2H), 2.24-2.33 (m, 3H). LCMS: [M+H]⁺=403.34.

### Example 374: 6-(2-amino-5-(3-((dimethylamino)methyl)-4-morpholinophenyl)-6-fluoropyridin-3-yl)-3,4-dihydroisoquinolin-1(2H)-one (I-432)

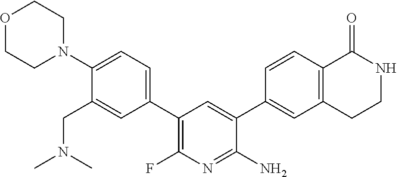 [SMILES: CN(C)Cc1cc(-c2cc(-c3ccc4c(c3)CCNC4=O)c(N)nc2F)ccc1N1CCOCC1]

Step 1: 1-(5-bromo-2-morpholinophenyl)-N,N-dimethylmethanamine

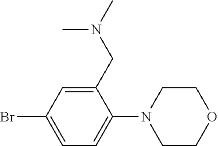 [SMILES: CN(C)Cc1cc(Br)ccc1N1CCOCC1]

Dimethylamine, 2.0M solution in THF (3.70 ml, 7.40 mmol) was added to a solution of 5-bromo-2-morpholinobenzaldehyde (500 mg, 1.85 mmol), and glacial acetic acid (10.59 μl, 0.185 mmol) in CH₂Cl₂ (10 mL) at RT. After 5 min of stirring NaBH(OAC)₃ (1569 mg, 7.40 mmol) was added and the suspension was stirred for 30 min at RT. The reaction mixture was basified with 1 N NaOH aqueous solution. The organic phase was separated, and aqueous phase was extracted with CH₂Cl₂ (5 mL). The combined org phase was dried over Na₂SO₄ and concentrated to yield the crude product as a pale-yellow solid (543 mg, 98%). LCMS: [M+H]⁺=299.92.

Step 2: 6-(2-amino-5-(3-((dimethylamino)methyl)-4-morpholinophenyl)-6-fluoropyridin-3-yl)-3,4-dihydroisoquinolin-1(2H)-one

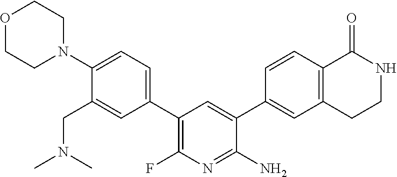 [SMILES: CN(C)Cc1cc(-c2cc(-c3ccc4c(c3)CCNC4=O)c(N)nc2F)ccc1N1CCOCC1]

Prepared from 1-(5-bromo-2-morpholinophenyl)-N,N-dimethylmethanamine (60 mg, 0.201 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (100 mg, 0.261 mmol) to yield the title compound as a beige solid (63 mg, 63% yield). ¹H NMR (500 MHz, Methanol-d₄) δ ppm 7.92-7.97 (m, 1H), 7.57-7.63 (m, 1H), 7.48-7.52 (m, 1H), 7.48-7.51 (m, 1H), 7.41 (dd, J=8.07, 1.59 Hz, 1H), 7.34-7.39 (m, 2H), 7.13 (d, J=8.44 Hz, 1H), 3.73-3.79 (m, 4H), 3.53-3.59 (m, 2H), 3.45 (t, J=6.66 Hz, 2H), 2.96 (t, J=6.66 Hz, 2H), 2.79-2.87 (m, 4H), 2.19 (s, 6H). LCMS: [M+H]⁺=476.42.

### Example 375: 6-(2-amino-5-(3-((dimethylamino)methyl)-4-morpholinophenyl)-6-fluoropyridin-3-yl)-7-fluoro-3,4-dihydroisoquinolin-1(2H)-one (I-433)

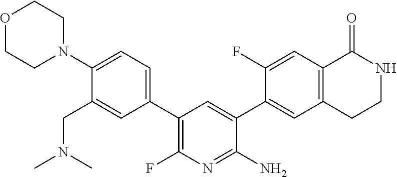 [SMILES: CN(C)Cc1cc(-c2cc(-c3cc4c(cc3F)C(=O)NCC4)c(N)nc2F)ccc1N1CCOCC1]

Prepared from 1-(5-bromo-2-morpholinophenyl)-N,N-dimethylmethanamine (60 mg, 0.201 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (105 mg, 0.261 mmol) to afford the title compound as a beige solid (46 mg, 44% yield). ¹H NMR (500 MHz, Methanol-d₄) δ ppm 8.09 (br. s., 1H), 7.60 (dd, J=12.96, 10.15 Hz, 2H), 7.50 (s, 1H), 7.41 (dd, J=16.44, 7.76 Hz, 2H), 7.12 (d, J=8.44 Hz, 1H), 6.25 (s, 2H), 3.70-3.78 (m, 4H), 3.44 (s, 2H), 3.40 (td, J=6.42, 2.69 Hz, 2H), 2.89-2.97 (m, 6H), 2.18 (s, 6H). LCMS: [M+H]⁺=494.41.

### Example 376: 6-(2-amino-5-(2,5-dimethyl-1,2,3,4-tetrahydroisoquinolin-7-yl)-6-fluoropyridin-3-yl)-3,4-dihydroisoquinolin-1(2H)-one (I-434)

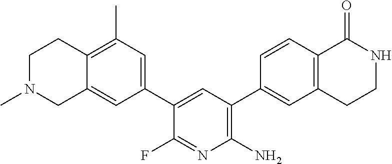 [SMILES: Cc1cc(-c2cc(-c3ccc4c(c3)CCNC4=O)c(N)nc2F)cc2c1CCN(C)C2]

Step 1: 7-bromo-2,5-dimethyl-1,2,3,4-tetrahydroisoquinoline

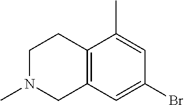

Prepared using 7-bromo-5-methyl-1,2,3,4-tetrahydroisoquinoline (40 mg, 0.177 mmol) and formaldehyde solution, 37% wt in water (0.040 mL, 0.531 mmol) to give the product as a pale-yellow solid (38 mg, 89% yield). LCMS: [M+H]⁺=239.96.

Step 2: Preparation of 6-(2-amino-5-(2,5-dimethyl-1,2,3,4-tetrahydroisoquinolin-7-yl)-6-fluoropyridin-3-yl)-3,4-dihydroisoquinolin-1(2H)-one

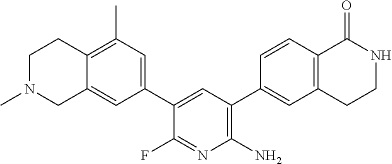 [SMILES: Cc1cc(-c2cc(-c3ccc4c(c3)CCNC4=O)c(N)nc2F)cc2c1CCN(C)C2]

Prepared from 7-bromo-2,5-dimethyl-1,2,3,4-tetrahydroisoquinoline (38 mg, 0.158 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (76 mg, 0.198 mmol) to afford the title compound as an off white solid (36 mg, 52% yield). ¹H NMR (500 MHz, METHANOL-d₄) δ ppm 8.01-8.10 (m, 1H), 7.60-7.67 (m, 1H), 7.51 (dd, J=7.95, 1.47 Hz, 1H), 7.46 (s, 1H), 7.21 (s, 1H), 7.09 (s, 1H), 3.65 (s, 2H), 3.56 (t, J=6.66 Hz, 2H), 3.07 (t, J=6.66 Hz, 2H), 2.83 (dd, J=11.00, 4.65 Hz, 4H), 2.48 (s, 3H), 2.28 (s, 3H); LCMS: [M+H]⁺=417.44.

### Example 377: 7-(2-amino-6-fluoro-5-(4-(4-isopropylpiperazin-1-yl)phenyl)pyridin-3-yl)-2-(trifluoromethyl)quinazolin-4(3H)-one (I-435)

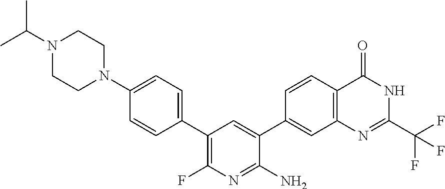 [SMILES: CC(C)N1CCN(c2ccc(-c3cc(-c4ccc5c(=O)[nH]c(C(F)(F)F)nc5c4)c(N)nc3F)cc2)CC1]

Step 1: 7-chloro-2-(trifluoromethyl)quinazolin-4(3H)-one

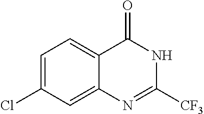 [SMILES: O=c1[nH]c(C(F)(F)F)nc2cc(Cl)ccc12]

To a 0° C. solution of 2-amino-4-chlorobenzoic acid (300 mg, 1.748 mmol) and N,N-diisopropylethylamine (1.523 ml, 8.74 mmol) in 1,2-dichloroethane (5 ml) was added trifluoroacetic anhydride (0.729 ml, 5.25 mmol). After addition, the reaction was heated at 50° C. for 1 hour. The reaction was concentrated in vacuo and ammonia solution, 7 N in methanol (2.498 ml, 17.48 mmol) was added. The reaction was heated at 80° C. overnight. The reaction was concentrated onto celite and purified by flash chromatography (Biotage, silica gel) eluting with 0-100% EtOAc/Hexanes. The desired fractions were collected, concentrated and dried under vacuum to afford the product (66.7 mg, 15% yield) as a yellow solid. ¹H NMR (500 MHz, DMSO-d₆) δ ppm 13.74 (br. s., 1H), 8.18 (d, J=8.56 Hz, 1H), 7.92 (d, J=1.96 Hz, 1H), 7.71 (dd, J=8.50, 2.02 Hz, 1H); LCMS: [M+H]⁺=249.19.

Step 2: 7-(2-amino-6-fluoro-5-(4-(4-isopropylpiperazin-1-yl)phenyl)pyridin-3-yl)-2-(trifluoromethyl)quinazolin-4(3H)-one

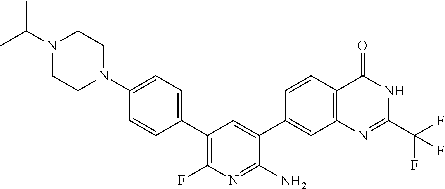 [SMILES: CC(C)N1CCN(c2ccc(-c3cc(-c4ccc5c(=O)[nH]c(C(F)(F)F)nc5c4)c(N)nc3F)cc2)CC1]

The title compound was prepared from 7-chloro-2-(trifluoromethyl)quinazolin-4(3H)-one (43 mg, 0.147 mmol) and 6-fluoro-5-(4-(4-isopropylpiperazin-1-yl)phenyl)-3-(4,4,5,5-tetramethyl-1,3,2-dioxaborolan-2-yl)pyridin-2-amine (78 mg, 0.176 mmol) to give the title compound (2.2 mg, 4.20 μmol, 2.86% yield) as an off-white solid. ¹H NMR (500 MHz, DMSO-d₆) δ ppm 8.17 (d, J=8.07 Hz, 1H), 7.80 (br. s., 1H), 7.66 (d, J=10.03 Hz, 2H), 7.46 (d, J=8.07 Hz, 2H), 7.01 (d, J=8.68 Hz, 2H), 6.24 (br. s., 2H), 2.95 (br. s., 4H), 1.15 (d, J=3.42 Hz, 6H); LCMS: [M+H]⁺=527.31.

### Example 379: 6-(2-amino-5-(3-((dimethylamino)methyl)-4-(tetrahydro-2H-pyran-4-yl)phenyl)-6-fluoropyridin-3-yl)-8-fluoro-3,4-dihydroisoquinolin-1(2H)-one (I-436)

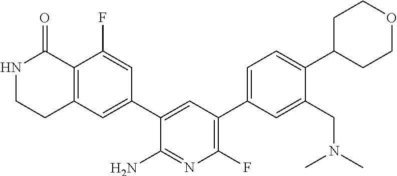 [SMILES: CN(C)Cc1cc(-c2cc(-c3cc(F)c4c(c3)CCNC4=O)c(N)nc2F)ccc1C1CCOCC1]

Prepared from N,N-dimethyl-1-(2-(tetrahydro-2H-pyran-4-yl)-5-(4,4,5,5-tetramethyl-1,3,2-dioxaborolan-2-yl)phenyl)methanamine (61 mg, 0.176 mmol) and 6-(2-amino-5-bromo-6-fluoropyridin-3-yl)-8-fluoro-3,4-dihydroisoquinolin-1(2H)-one (50 mg, 0.141 mmol) to yield the title compound as an off white solid (46 mg, 63% yield). ¹H NMR (500 MHz, METHANOL-d₄) δ ppm 7.72 (d, J=9.66 Hz, 1H), 7.48 (d, J=8.19 Hz, 1H), 7.46 (s, 1H), 7.39 (d, J=8.07 Hz, 1H), 7.32 (s, 1H), 7.28 (d, J=11.98 Hz, 1H), 4.07 (dd, J=11.19, 3.73 Hz, 2H), 3.61 (t, J=11.19 Hz, 2H), 3.57 (s, 2H), 3.51 (t, J=6.42 Hz, 2H), 3.23-3.30 (m, 1H), 3.06 (t, J=6.42 Hz, 2H), 2.25-2.36 (m, 6H), 1.81-1.93 (m, 2H), 1.73 (d, J=12.59 Hz, 2H); LCMS: [M+H]⁺=493.34.

### Example 380: 7-(2-amino-5-(3-((dimethylamino)methyl)-4-(tetrahydro-2H-pyran-4-yl)phenyl)-6-fluoropyridin-3-yl)-2-methylquinazolin-4(3H)-one (I-437)

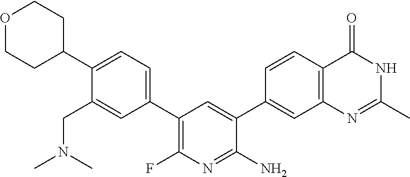 [SMILES: Cc1nc2cc(-c3cc(-c4ccc(C5CCOCC5)c(CN(C)C)c4)c(F)nc3N)ccc2c(=O)[nH]1]

Prepared from N,N-dimethyl-1-(2-(tetrahydro-2H-pyran-4-yl)-5-(4,4,5,5-tetramethyl-1,3,2-dioxaborolan-2-yl)phenyl)methanamine (58 mg, 0.168 mmol) and 7-(2-amino-5-bromo-6-fluoropyridin-3-yl)-2-methylquinazolin-4(3H)-one (60 mg, 0.134 mmol) to yield the title compound as a beige solid (32 mg, 46.5% yield). ¹H NMR (500 MHz, METHANOL-d4) δ ppm 8.29 (d, J=8.19 Hz, 1H), 7.73-7.79 (m, 2H), 7.72-7.79 (m, 2H), 7.66 (dd, J=8.19, 1.59 Hz, 1H), 7.45-7.53 (m, 2H), 7.45-7.53 (m, 2H), 7.40 (d, J=8.19 Hz, 1H), 4.07 (dd, J=11.13, 3.79 Hz, 2H), 3.61 (t, J=11.00 Hz, 2H), 3.55-3.58 (m, 2H), 3.57 (s, 2H), 3.24-3.30 (m, 1H), 2.49 (s, 3H), 2.28-2.32 (m, 6H), 1.81-1.92 (m, 2H), 1.73 (d, J=11.49 Hz, 2H). LCMS: [M+H]⁺=488.33.

### Example 381: 6-(2-amino-6-fluoro-5-(6-methoxy-2-methyl-1,2,3,4-tetrahydroisoquinolin-7-yl)pyridin-3-yl)-3,4-dihydroisoquinolin-1(2H)-one(I-438)

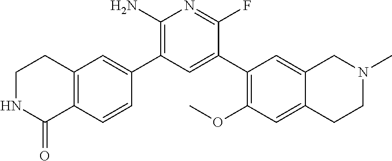 [SMILES: COc1cc2c(cc1-c1cc(-c3ccc4c(c3)CCNC4=O)c(N)nc1F)CN(C)CC2]

Step 1: Preparation of 7-bromo-6-methoxy-2-methyl-1,2,3,4-tetrahydroisoquinoline

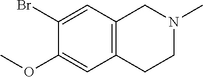

To an acetonitrile (12 mL) solution of 6-methoxy-2-methyl-1,2,3,4-tetrahydroisoquinolin-7-amine (200 mg, 1.04 mmol), copper(I) bromide (14.92 mg, 0.104 mmol), tert-butyl nitrite, >=90% (GC) (129 mg, 1.248 mmol) and tetrabutylammonium bromide (671 mg, 2.08 mmol) at RT was added camphor-10-sulfonic acid, 98% (290 mg, 1.25 mmol) and the reaction mixture was stirred at RT for 2 h. The reaction mixture was quenched with 2 ml water and the volatiles were removed. The residue was partitioned between 5 ml water and 4 ml CH₂Cl₂, the organic phase was separated, aqueous phase was extracted with CH₂Cl₂ (3×4 mL), the combined org phase was washed with brine, dried over Na₂SO₄ and concentrated. Purification by silica gel chromatography, eluting with CH₂Cl₂ containing 0-2% MeOH and 0-0.2% NH₄OH, followed by filtration through a porapak cation exchange resin cartridge yielded the title compound as a brown oil (98 mg, 18% yield, 50% purity). LCMS: [M+H]⁺=256.21

Step 2: 6-(2-amino-6-fluoro-5-(6-methoxy-2-methyl-1,2,3,4-tetrahydroisoquinolin-7-yl)pyridin-3-yl)-3,4-dihydroisoquinolin-1(2H)-one

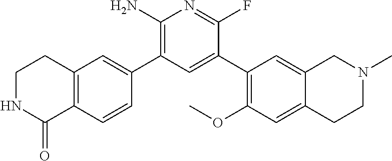 [SMILES: COc1cc2c(cc1-c1cc(-c3ccc4c(c3)CCNC4=O)c(N)nc1F)CN(C)CC2]

Prepared from 7-bromo-6-methoxy-2-methyl-1,2,3,4-tetrahydroisoquinoline (98 mg, 0.191 mmol, 50% purity) and 6-(2-amino-6-fluoro-5-(4,4,5,5-tetramethyl-1,3,2-dioxaborolan-2-yl)pyridin-3-yl)-3,4-dihydroisoquinolin-1(2H)-one (95 mg, 0.249 mmol) to give the title compound as an off white solid (39 mg, 45% yield). ¹H NMR (500 MHz, METHANOL-d₄) δ ppm 7.92 (d, J=7.95 Hz, 1H), 7.34-7.41 (m, 2H), 7.32 (s, 1H), 6.85 (s, 1H), 6.68-6.75 (m, 1H), 3.67 (s, 3H), 3.47 (s, 2H), 3.42-3.46 (m, 2H), 3.44 (t, J=6.66 Hz, 2H), 2.94 (t, J=6.60 Hz, 2H), 2.85-2.91 (m, 2H), 2.66 (t, J=5.99 Hz, 2H), 2.36 (s, 3 H); LCMS: [M+H]⁺=433.30.

### Example 383: 7-(2-amino-6-fluoro-5-(4-(4-isopropylpiperazin-1-yl)phenyl)pyridin-3-yl)-6-fluoro-2-methylquinazolin-4(3H)-one (I-439)

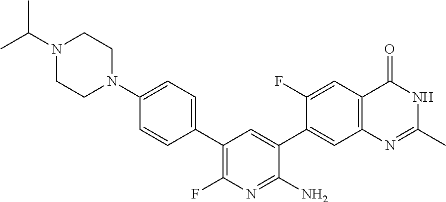 [SMILES: Cc1nc2cc(-c3cc(-c4ccc(N5CCN(C(C)C)CC5)cc4)c(F)nc3N)c(F)cc2c(=O)[nH]1]

Step 1: 7-bromo-6-fluoro-2-methylquinazolin-4(3H)-one

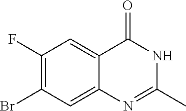

2-Amino-4-bromo-5-fluorobenzoic acid (1.0 g, 4.27 mmol) was taken up in acetic anhydride (5.65 ml, 59.8 mmol) and heated at 130° C. overnight. The reaction mixture was cooled to RT and left there for two hours. The precipitated solid was filtered, washed with cold ether, and dried under vacuum to give 2-acetamido-4-bromo-5-fluorobenzoic acid which was used in the next step without further purification. LCMS [M−H]⁻ 273.89. The 2-acetamido-4-bromo-5-fluorobenzoic acid was take up in 27% aqueous ammonium hydroxide solution (3.33 ml, 23.07 mmol) and heated in a sealed tube at 80° C. for 4 hours. After cooling to RT, the solid was filtered, washed with water, and dried under vacuum to afford 7-bromo-6-fluoro-2-methylquinazolin-4(3H)-one (132 mg, 0.513 mmol, 12.02% yield) as a white solid. ¹H NMR (500 MHz, DMSO-d₆) δ ppm 12.42 (br. s., 1H), 7.96 (d, J=6.24 Hz, 1H), 7.87 (d, J=8.56 Hz, 1H), 2.35 (s, 3H); LCMS: [M+H]⁺ 257.09.

Step 2: (6-fluoro-2-methyl-4-oxo-3,4-dihydroquinazolin-7-yl)boronic acid

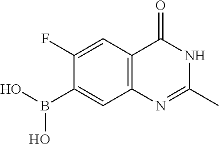 [SMILES: Cc1nc2cc(B(O)O)c(F)cc2c(=O)[nH]1]

The product was prepared from 7-bromo-6-fluoro-2-methylquinazolin-4(3H)-one (86 mg, 0.335 mmol) to give a mixture of boronate and boronic acid which was used in the next step without further purification. LCMS: [M+H]⁺ 289.31 Boronate, 207.18 Boronic acid.

Step 3: 7-(2-amino-6-fluoro-5-(4-(4-isopropylpiperazin-1-yl)phenyl)pyridin-3-yl)-6-fluoro-2-methylquinazolin-4(3H)-one

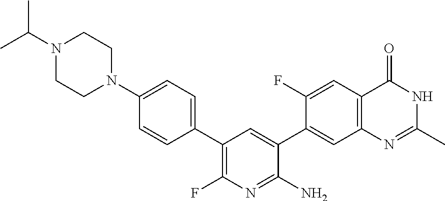 [SMILES: Cc1nc2cc(-c3cc(-c4ccc(N5CCN(C(C)C)CC5)cc4)c(F)nc3N)c(F)cc2c(=O)[nH]1]

Prepared from (6-fluoro-2-methyl-4-oxo-3,4-dihydroquinazolin-7-yl)boronic acid (35.9 mg, 0.162 mmol) and 3-bromo-6-fluoro-5-(4-(4-isopropylpiperazin-1-yl)phenyl)pyridin-2-amine (53 mg, 0.135 mmol) to give the title compound (47.2 mg, 71.4% yield) as a yellow solid. ¹H NMR (500 MHz, DMSO-d₆) δ ppm 12.32 (br. s., 1H), 7.81 (d, J=9.41 Hz, 1H), 7.59-7.68 (m, 2H), 7.39 (d, J=7.95 Hz, 2H), 6.96 (d, J=8.80 Hz, 2H), 6.22 (s, 2H), 3.14 (d, J=4.65 Hz, 4H), 2.63-2.71 (m, 1H), 2.57 (br. s., 4H), 2.37 (s, 3H), 1.00 (d, J=6.48 Hz, 6H); LCMS: [M+H]⁺=491.36.

### Example 385: 6-(2-amino-5-(4-(4-(4,4-difluorobutyl)piperazin-1-yl)phenyl)-6-fluoropyridin-3-yl)-8-fluoro-3,4-dihydroisoquinolin-1(2H)-one (I-440)

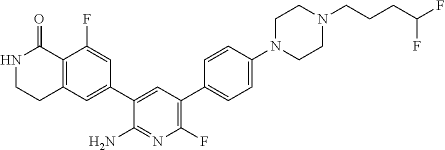 [SMILES: Nc1nc(F)c(-c2ccc(N3CCN(CCCC(F)F)CC3)cc2)cc1-c1cc(F)c2c(c1)CCNC2=O]

Step 1: Preparation of 6-(2-amino-6-fluoro-5-(4-(piperazin-1-yl)phenyl)pyridin-3-yl)-8-fluoro-3,4-dihydroisoquinolin-1(2H)-one

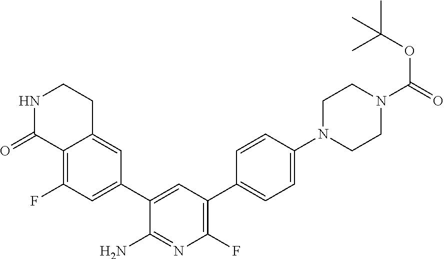 [SMILES: CC(C)(C)OC(=O)N1CCN(c2ccc(-c3cc(-c4cc(F)c5c(c4)CCNC5=O)c(N)nc3F)cc2)CC1]

Prepared from 6-(2-amino-5-bromo-6-fluoropyridin-3-yl)-8-fluoro-3,4-dihydroisoquinolin-1(2H)-one (150 mg, 0.424 mmol) and tert-butyl 4-[4-(4,4,5,5-tetramethyl-1,3,2-dioxaborolan-2-yl)phenyl]tetrahydro-1(2H)-pyrazinecarboxylate (206 mg, 0.529 mmol). The title compound was isolated as a beige solid (205 mg, 97% yield). LCMS: [M+H]⁺=536.34

Step 2: 6-(2-amino-6-fluoro-5-(4-(piperazin-1-yl)phenyl)pyridin-3-yl)-8-fluoro-3,4-dihydroisoquinolin-1(2H)-one

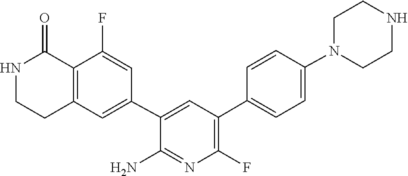 [SMILES: Nc1nc(F)c(-c2ccc(N3CCNCC3)cc2)cc1-c1cc(F)c2c(c1)CCNC2=O]

Prepared by deprotecting tert-butyl 4-(4-(6-amino-2-fluoro-5-(8-fluoro-1-oxo-1,2,3,4-tetrahydroisoquinolin-6-yl)pyridin-3-yl)phenyl)piperazine-1-carboxylate (205 mg, 0.371 mmol) with trifluoroacetic acid (2.56 mL, 33.4 mmol). The title compound was isolated as a beige solid (163 mg, quantitative yield). LCMS: [M+H]⁺=436.31

Step 3: 6-(2-amino-5-(4-(4-(4,4-difluorobutyl)piperazin-1-yl)phenyl)-6-fluoropyridin-3-yl)-8-fluoro-3,4-dihydroisoquinolin-1(2H)-one

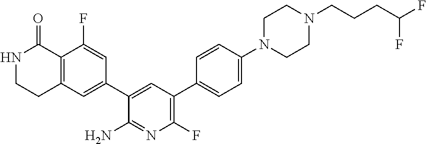 [SMILES: Nc1nc(F)c(-c2ccc(N3CCN(CCCC(F)F)CC3)cc2)cc1-c1cc(F)c2c(c1)CCNC2=O]

Prepared from 6-(2-amino-6-fluoro-5-(4-(piperazin-1-yl)phenyl)pyridin-3-yl)-8-fluoro-3,4-dihydroisoquinolin-1(2H)-one (50 mg, 0.115 mmol) and 4-bromo-1,1-difluorobutane (59.6 mg, 0.344 mmol) to afford the product as an off-white solid (46 mg, 72%). ¹H NMR (500 MHz, DMSO-d₆) δ ppm 7.98 (br. s., 1H), 7.58-7.69 (m, 1H), 7.38-7.47 (m, 2H), 7.30 (s, 1H), 7.26 (d, J=12.10 Hz, 1H), 6.92-7.04 (m, 2H), 6.28 (s, 2H), 5.99-6.25 (m, 1H), 3.35 (d, J=2.20 Hz, 2H), 3.17 (br. s., 4H), 2.94 (t, J=6.24 Hz, 2H), 2.52-2.56 (m, 4H), 2.35-2.42 (m, 2H), 1.77-1.96 (m, 2H), 1.59 (quin, J=7.37 Hz, 2H) LCMS: [M+H]⁺=528.37

### Example 386: 6-(2-amino-5-(4-(4-(4,4-difluorobutyl)piperazin-1-yl)phenyl)-6-fluoropyridin-3-yl)-7-fluoro-3,4-dihydroisoquinolin-1(2H)-one (I-441)

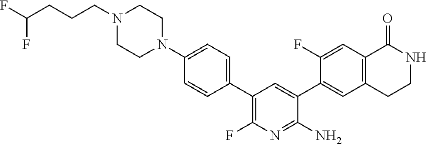 [SMILES: Nc1nc(F)c(-c2ccc(N3CCN(CCCC(F)F)CC3)cc2)cc1-c1cc2c(cc1F)C(=O)NCC2]

Step 1: tert-butyl 4-(4-(6-amino-2-fluoro-5-(7-fluoro-1-oxo-1,2,3,4-tetrahydroisoquinolin-6-yl)pyridin-3-yl)phenyl)piperazine-1-carboxylate

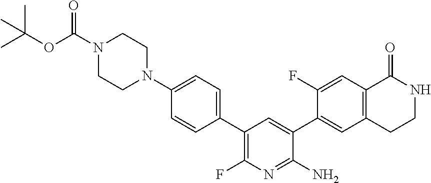 [SMILES: CC(C)(C)OC(=O)N1CCN(c2ccc(-c3cc(-c4cc5c(cc4F)C(=O)NCC5)c(N)nc3F)cc2)CC1]

Prepared from 6-(2-amino-5-bromo-6-fluoropyridin-3-yl)-7-fluoro-3,4-dihydroisoquinolin-1(2H)-one (500 mg, 1.412 mmol) and tert-butyl 4-[4-(4,4,5,5-tetramethyl-1,3,2-dioxaborolan-2-yl)phenyl]tetrahydro-1(2H)-pyrazinecarboxylate (685 mg, 1.765 mmol) to give the title compound as a beige solid (638 mg, 84% yield). LCMS: [M+H]⁺=536.40

Step 2: 6-(2-amino-6-fluoro-5-(4-(piperazin-1-yl)phenyl)pyridin-3-yl)-7-fluoro-3,4-dihydroisoquinolin-1(2H)-one, Trifluoroacetic Acid, CF3COOH salt

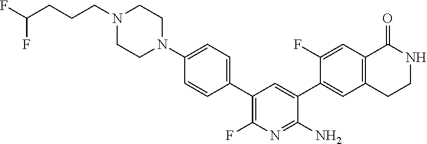 [SMILES: Nc1nc(F)c(-c2ccc(N3CCN(CCCC(F)F)CC3)cc2)cc1-c1cc2c(cc1F)C(=O)NCC2]

Prepared from tert-butyl 4-(4-(6-amino-2-fluoro-5-(7-fluoro-1-oxo-1,2,3,4-tetrahydroisoquinolin-6-yl)pyridin-3-yl)phenyl)piperazine-1-carboxylate (201 mg, 0.375 mmol) with Trifluoroacetic acid (2.59 ml, 33.8 mmol). The title compound was isolated as an off white solid (189 mg, 92%). LCMS: [M+H]+=436.31

Step 3: 6-(2-amino-5-(4-(4-(4,4-difluorobutyl)piperazin-1-yl)phenyl)-6-fluoropyridin-3-yl)-7-fluoro-3,4-dihydroisoquinolin-1(2H)-one

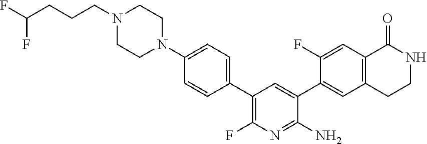 [SMILES: Nc1nc(F)c(-c2ccc(N3CCN(CCCC(F)F)CC3)cc2)cc1-c1cc2c(cc1F)C(=O)NCC2]

Prepared from 6-(2-amino-6-fluoro-5-(4-(piperazin-1-yl)phenyl)pyridin-3-yl)-7-fluoro-3,4-dihydroisoquinolin-1(2H)-one (55 mg, 0.100 mmol) and 4-bromo-1,1-difluorobutane (51.9 mg, 0.300 mmol) to give the title compound as a white solid (40 mg, 72% yield). ¹H NMR (500 MHz, DMSO-d₆) δ ppm 8.10 (br. s., 1H), 7.60 (dd, J=13.33, 10.27 Hz, 2H), 7.43 (d, J=7.09 Hz, 1H), 7.39 (d, J=8.19 Hz, 2H), 6.97 (d, J=8.68 Hz, 2H), 6.00-6.26 (m, 3H), 3.39-3.44 (m, 2H), 3.16 (d, J=4.52 Hz, 4H), 2.93 (t, J=6.30 Hz, 2H), 2.52-2.61 (m, 4H), 2.38 (t, J=6.97 Hz, 2H), 1.78-1.93 (m, 2H), 1.59 (quin, J=7.37 Hz, 2H); LCMS: [M+H]⁺=528.30.

### Example 387: tert-butyl 4-(4-(6-amino-2-fluoro-5-(1-oxo-1,2,3,4-tetrahydroisoquinolin-6-yl)pyridin-3-yl)-2-((dimethylamino)methyl)phenyl)piperazine-1-carboxylate (I-442)

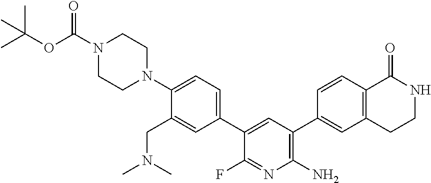 [SMILES: CN(C)Cc1cc(-c2cc(-c3ccc4c(c3)CCNC4=O)c(N)nc2F)ccc1N1CCN(C(=O)OC(C)(C)C)CC1]

Step 1: tert-butyl 4-(4-bromo-2-formylphenyl)piperazine-1-carboxylate

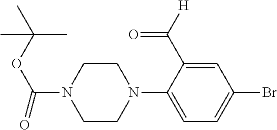 [SMILES: CC(C)(C)OC(=O)N1CCN(c2ccc(Br)cc2C=O)CC1]

Prepared from 5-bromo-2-fluorobenzaldehyde (4 g, 19.70 mmol) and 1-Boc-piperazine (5.50 g, 29.6 mmol). The title compound was isolated as a pale-yellow oil (4.18 mg, 57% yield). LCMS: [M+H]⁺=369.31.

Step 2: tert-butyl 4-(4-bromo-2-((dimethylamino)methyl)phenyl)piperazine-1-carboxylate

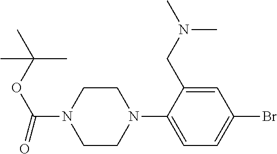 [SMILES: CN(C)Cc1cc(Br)ccc1N1CCN(C(=O)OC(C)(C)C)CC1]

Prepared from tert-butyl 4-(4-bromo-2-formylphenyl)piperazine-1-carboxylate (0.495 g, 1.338 mmol) and dimethylamine, 2.0M solution in THF (5.35 ml, 10.70 mmol). The title compound was isolated as a pale-yellow oil (321 mg, 60% yield). LCMS: [M+H]⁺=398.30.

Step 3: Preparation of tert-butyl 4-(4-(6-amino-2-fluoro-5-(1-oxo-1,2,3,4-tetrahydroisoquinolin-6-yl)pyridin-3-yl)-2-((dimethylamino)methyl)phenyl)piperazine-1-carboxylate

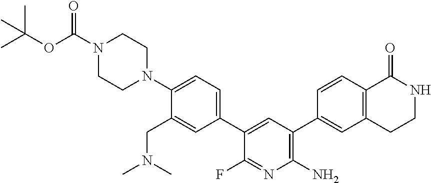 [SMILES: CN(C)Cc1cc(-c2cc(-c3ccc4c(c3)CCNC4=O)c(N)nc2F)ccc1N1CCN(C(=O)OC(C)(C)C)CC1]

Prepared from tert-butyl 4-(4-bromo-2-((dimethylamino)methyl)phenyl)piperazine-1-carboxylate (56 mg, 0.141 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (67.3 mg, 0.176 mmol) to give the title compound as an off white solid (45 mg, 53% yield). ¹H NMR (500 MHz, METHANOL-d₄) δ ppm 8.02-8.10 (m, 1H), 7.71 (d, J=9.78 Hz, 1H), 7.59-7.64 (m, 1H), 7.53 (d, J=7.95 Hz, 1H), 7.46-7.51 (m, 2H), 7.22 (d, J=8.44 Hz, 1H), 3.66 (s, 2H), 3.61 (br. s., 4H), 3.52-3.57 (m, 2H), 3.05-3.12 (m, 2H), 2.88-2.96 (m, 4H), 2.30 (s, 6H), 1.49-1.56 (m, 9H); LCMS: [M+H]+=575.32.

### Example 388: (S)-6-(2-amino-6-fluoro-5-(3-(1-methylpyrrolidin-2-yl)phenyl)pyridin-3-yl)-3,4-dihydroisoquinolin-1(2H)-one (I-443)

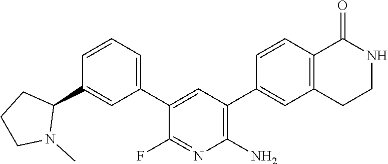 [SMILES: CN1CCC[C@H]1c1cccc(-c2cc(-c3ccc4c(c3)CCNC4=O)c(N)nc2F)c1]

Step 1: (S)-2-(3-bromophenyl)-1-methylpyrrolidine

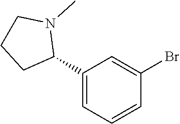

Prepared from (2S)-2-(3-bromophenyl)pyrrolidine (60 mg, 0.265 mmol) and formaldehyde solution, 37% wt in water (0.059 mL, 0.796 mmol). The title compound was isolated as a colorless oil (54 mg, 85% yield). LCMS: [M+H]⁺=240.18

Step 2: (S)-6-(2-amino-6-fluoro-5-(3-(1-methylpyrrolidin-2-yl)phenyl)pyridin-3-yl)-3,4-dihydroisoquinolin-1(2H)-one

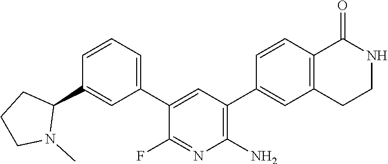 [SMILES: CN1CCC[C@H]1c1cccc(-c2cc(-c3ccc4c(c3)CCNC4=O)c(N)nc2F)c1]

Prepared from (S)-2-(3-bromophenyl)-1-methylpyrrolidine (27 mg, 0.112 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (53.9 mg, 0.141 mmol) to afford the title compound as a beige solid (26 mg, 50% yield). ¹H NMR (500 MHz, METHANOL-d4) δ ppm 7.92-7.97 (m, 1 H), 7.59 (d, J=9.78 Hz, 1H), 7.40-7.45 (m, 2H), 7.35-7.39 (m, 2H), 7.27-7.32 (m, 1H), 7.18-7.23 (m, 1H), 7.18-7.23 (m, 1H), 3.45 (t, J=6.66 Hz, 2H), 3.08-3.16 (m, 2H), 2.92-2.97 (m, 2H), 2.23-2.31 (m, 1H), 2.13-2.19 (m, 1H), 2.07-2.12 (m, 3H), 1.85-1.93 (m, 1H), 1.73-1.82 (m, 2H); LCMS: [M+H]⁺=416.88.

### Example 389: (S)-6-(2-amino-6-fluoro-5-(3-(1-methylpyrrolidin-2-yl)phenyl)pyridin-3-yl)-7-fluoro-3,4-dihydroisoquinolin-1(2H)-one (I-444)

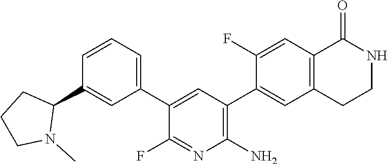 [SMILES: CN1CCC[C@H]1c1cccc(-c2cc(-c3cc4c(cc3F)C(=O)NCC4)c(N)nc2F)c1]

Prepared from (S)-2-(3-bromophenyl)-1-methylpyrrolidine (27 mg, 0.112 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (56.4 mg, 0.141 mmol) to afford the title compound as a beige solid 9 mg, 17.5% yield). ¹H NMR (500 MHz, METHANOL-d₄) δ ppm 7.76 (d, J=10.03 Hz, 1H), 7.68-7.72 (m, 1H), 7.54 (s, 1H), 7.45-7.49 (m, 1H), 7.39-7.45 (m, 2 H), 7.32 (d, J=7.46 Hz, 1H), 3.56 (t, J=6.66 Hz, 2H), 3.16-3.24 (m, 2H), 3.04 (t, J=6.54 Hz, 2H), 2.38 (q, J=9.25 Hz, 1H), 2.25-2.32 (m, 1H), 2.22 (s, 3H), 1.96-2.05 (m, 1H), 1.85-1.92 (m, 2H); LCMS: [M+H]⁺=435.31

### Example 390: 6-(2-amino-5-(4-(4-(4,4-difluorobutyl)piperazin-1-yl)-3-((dimethylamino)methyl)phenyl)-6-fluoropyridin-3-yl)-3,4-dihydroisoquinolin-1(2H)-one (I-445)

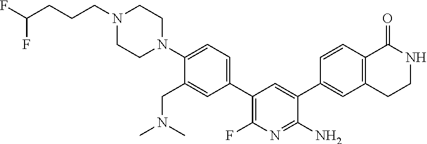 [SMILES: CN(C)Cc1cc(-c2cc(-c3ccc4c(c3)CCNC4=O)c(N)nc2F)ccc1N1CCN(CCCC(F)F)CC1]

Step 1: Preparation of 6-(2-amino-6-fluoro-5-(4-(piperazin-1-yl)phenyl)pyridin-3-yl)-8-fluoro-3,4-dihydroisoquinolin-1(2H)-one

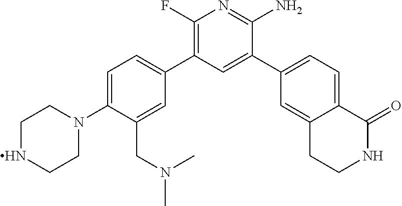

Prepared from tert-butyl 4-(4-(6-amino-2-fluoro-5-(1-oxo-1,2,3,4-tetrahydroisoquinolin-6-yl)pyridin-3-yl)-2-((dimethylamino)methyl)phenyl)piperazine-1-carboxylate (39 mg, 0.068 mmol) with Trifluoroacetic acid (0.520 ml, 6.79 mmol). The title compound was isolated as a beige solid (30 mg, 93% yield). LCMS: [M+H]⁺=475.34.

Step 2: 6-(2-amino-5-(4-(4-(4,4-difluorobutyl)piperazin-1-yl)-3-((dimethylamino)methyl)phenyl)-6-fluoropyridin-3-yl)-3,4-dihydroisoquinolin-1(2H)-one

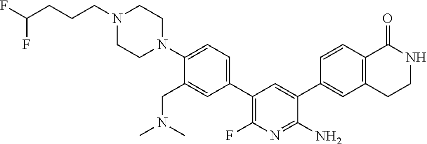 [SMILES: CN(C)Cc1cc(-c2cc(-c3ccc4c(c3)CCNC4=O)c(N)nc2F)ccc1N1CCN(CCCC(F)F)CC1]

Prepared from 6-(2-amino-5-(3-((dimethylamino)methyl)-4-(piperazin-1-yl)phenyl)-6-fluoropyridin-3-yl)-3,4-dihydroisoquinolin-1(2H)-one (30 mg, 0.063 mmol) and 4-bromo-1, 1-difluorobutane (16.40 mg, 0.095 mmol) to give the title compound as a beige solid (20 mg, 52% yield). ¹H NMR (500 MHz, METHANOL-d₄) δ ppm 8.06 (d, J=7.95 Hz, 1H), 7.71 (d, J=9.78 Hz, 1H), 7.60 (br. s., 1H), 7.53 (d, J=7.58 Hz, 1H), 7.44-7.50 (m, 2H), 7.18-7.28 (m, 1H), 5.83-6.10 (m, 1H), 3.63 (s, 2H), 3.57 (t, J=6.60 Hz, 2H), 3.08 (t, J=6.42 Hz, 2H), 3.00 (d, J=4.03 Hz, 4H), 2.68 (br. s., 4H), 2.48-2.56 (m, 2H), 2.23-2.34 (m, 6 H), 1.85-1.98 (m, 2H), 1.69-1.79 (m, 2H); LCMS: [M+H]⁺=567.23.

### Example 391: 6-(2-amino-5-(2-(4,4-difluorobutyl)-5-methyl-1,2,3,4-tetrahydroisoquinolin-7-yl)-6-fluoropyridin-3-yl)-3,4-dihydroisoquinolin-1(2H)-one (I-446)

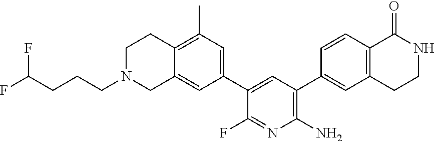 [SMILES: Cc1cc(-c2cc(-c3ccc4c(c3)CCNC4=O)c(N)nc2F)cc2c1CCN(CCCC(F)F)C2]

Prepared from 6-(2-amino-6-fluoro-5-(5-methyl-1,2,3,4-tetrahydroisoquinolin-7-yl)pyridin-3-yl)-3,4-dihydroisoquinolin-1(2H)-one (39 mg, 0.097 mmol) and 4-bromo-1,1-difluorobutane (33.5 mg, 0.194 mmol) to afford the title compound as a beige solid (35 mg, 69%). ¹H NMR (500 MHz, METHANOL-d₄) δ ppm 8.05 (d, J=7.95 Hz, 1H), 7.63 (d, J=9.78 Hz, 1H), 7.52 (d, J=7.95 Hz, 1H), 7.45-7.49 (m, 1H), 7.21 (s, 1H), 7.11 (s, 1H), 5.82-6.11 (m, 1H), 5.82-6.11 (m, 1H), 3.70 (s, 2H), 3.57 (t, J=6.60 Hz, 2H), 3.08 (t, J=6.54 Hz, 2H), 2.80-2.90 (m, 4H), 2.62 (d, J=7.83 Hz, 2H), 2.26-2.32 (m, 3H), 1.86-1.98 (m, 2H), 1.74-1.85 (m, 2H). LCMS: [M+H]⁺=495.22.

### Example 392: 6-(2-amino-5-(4-(4-(4,4-difluorobutyl)piperazin-1-yl)-3-((dimethylamino)methyl)phenyl)-6-fluoropyridin-3-yl)-7-fluoro-3,4-dihydroisoquinolin-1(2H)-one (I-447)

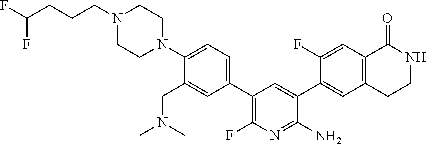 [SMILES: CN(C)Cc1cc(-c2cc(-c3cc4c(cc3F)C(=O)NCC4)c(N)nc2F)ccc1N1CCN(CCCC(F)F)CC1]

Step 1: tert-butyl 4-(4-(6-amino-2-fluoro-5-(7-fluoro-1-oxo-1,2,3,4-tetrahydroisoquinolin-6-yl)pyridin-3-yl)-2-((dimethylamino)methyl)phenyl)piperazine-1-carboxylate

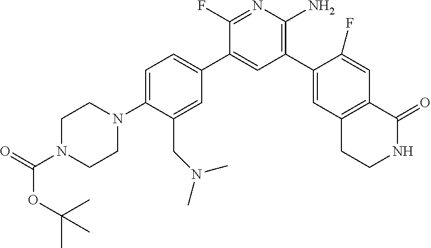 [SMILES: CN(C)Cc1cc(-c2cc(-c3cc4c(cc3F)C(=O)NCC4)c(N)nc2F)ccc1N1CCN(C(=O)OC(C)(C)C)CC1]

Prepared from tert-butyl 4-(4-bromo-2-((dimethylamino)methyl)phenyl)piperazine-1-carboxylate (58 mg, 0.146 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (73.0 mg, 0.182 mmol) to yield the title compound as a white solid (14 mg, 16% yield). LCMS: [M+H]⁺=593.30

Step 2: 6-(2-amino-5-(3-((dimethylamino)methyl)-4-(piperazin-1-yl)phenyl)-6-fluoropyridin-3-yl)-7-fluoro-3,4-dihydroisoquinolin-1(2H)-one

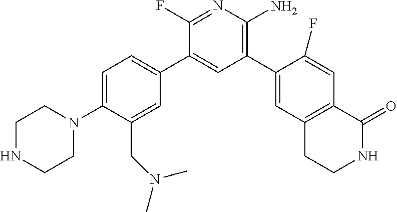 [SMILES: CN(C)Cc1cc(-c2cc(-c3cc4c(cc3F)C(=O)NCC4)c(N)nc2F)ccc1N1CCNCC1]

Prepared from tert-butyl 4-(4-(6-amino-2-fluoro-5-(7-fluoro-1-oxo-1,2,3,4-tetrahydroisoquinolin-6-yl)pyridin-3-yl)-2-((dimethylamino)methyl)phenyl)piperazine-1-carboxylate (14 mg, 0.024 mmol) and TFA (0.181 mL, 2.362 mmol) to give product as a beige solid (11.5 mg, 99% yield). LCMS: [M+H]⁺=493.32

Step 3: 6-(2-amino-5-(4-(4-(4,4-difluorobutyl)piperazin-1-yl)-3-((dimethylamino)methyl)phenyl)-6-fluoropyridin-3-yl)-7-fluoro-3,4-dihydroisoquinolin-1(2H)-one (

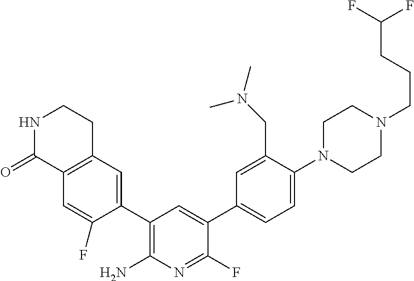 [SMILES: CN(C)Cc1cc(-c2cc(-c3cc4c(cc3F)C(=O)NCC4)c(N)nc2F)ccc1N1CCN(CCCC(F)F)CC1]

Prepared from 6-(2-amino-5-(3-((dimethylamino)methyl)-4-(piperazin-1-yl)phenyl)-6-fluoropyridin-3-yl)-7-fluoro-3,4-dihydroisoquinolin-1(2H)-one (11.5 mg, 0.023 mmol) and 4-bromo-1,1-difluorobutane (6.06 mg, 0.035 mmol) to give the title compound as an off white solid (11 mg, 77%). ¹H NMR (500 MHz, METHANOL-d4) δ ppm 7.76 (d, J=9.90 Hz, 1H), 7.70 (d, J=9.78 Hz, 1H), 7.59 (s, 1H), 7.47 (d, J=8.31 Hz, 1H), 7.38-7.44 (m, 1 H), 7.24 (d, J=8.31 Hz, 1H), 5.80-6.13 (m, 1H), 3.61-3.69 (m, 2H), 3.56 (t, J=6.66 Hz, 2 H), 3.04 (t, J=6.60 Hz, 2H), 3.00 (t, J=4.46 Hz, 4H), 2.69 (br. s., 4H), 2.47-2.56 (m, 2H), 2.23-2.36 (m, 6H), 1.84-1.98 (m, 2H), 1.66-1.79 (m, 2H); LCMS: [M+H]⁺=585.41.

### Example 393: 6-(2-amino-6-fluoro-5-(2-(2-methoxyethyl)-5-methyl-1,2,3,4-tetrahydroisoquinolin-7-yl)pyridin-3-yl)-3,4-dihydroisoquinolin-1(2H)-one (I-448)

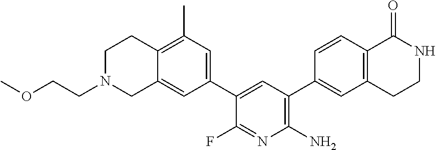 [SMILES: COCCN1CCc2c(C)cc(-c3cc(-c4ccc5c(c4)CCNC5=O)c(N)nc3F)cc2C1]

Prepared from 6-(2-amino-6-fluoro-5-(5-methyl-1,2,3,4-tetrahydroisoquinolin-7-yl)pyridin-3-yl)-3,4-dihydroisoquinolin-1(2H)-one (39 mg, 0.097 mmol) and 2-bromoethyl methyl ether (26.9 mg, 0.194 mmol) to give the title compound as an off white solid (21 mg, 45%). ¹H NMR (500 MHz, METHANOL-d4) δ ppm 8.02-8.08 (m, 1H), 7.58-7.64 (m, 1H), 7.48-7.53 (m, 1H), 7.45 (s, 1H), 7.17-7.22 (m, 1H), 7.04-7.10 (m, 1H), 3.71-3.76 (m, 2H), 3.65 (t, J=5.56 Hz, 2H), 3.56 (t, J=6.66 Hz, 2H), 3.39 (s, 3H), 3.06 (t, J=6.66 Hz, 2H), 2.88-2.93 (m, 2H), 2.80-2.84 (m, 2H), 2.78 (t, J=5.62 Hz, 2H), 2.27 (s, 3H); LCMS: [M+H]+=461.32

### Example 394: 6-(2-amino-5-(4-(2-cyclobutyl-2,7-diazaspiro[3.5]nonan-7-yl)phenyl)-6-fluoropyridin-3-yl)-7-fluoro-3,4-dihydroisoquinolin-1(2H)-one (I-449) TXX-4019-0047-02

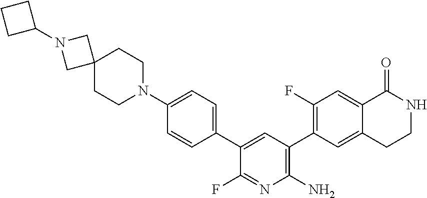 [SMILES: Nc1nc(F)c(-c2ccc(N3CCC4(CC3)CN(C3CCC3)C4)cc2)cc1-c1cc2c(cc1F)C(=O)NCC2]

Prepared from 6-(2-amino-5-bromo-6-fluoropyridin-3-yl)-3,4-dihydroisoquinolin-1(2H)-one (38.9 mg, 0.110 mmol) and 2-cyclobutyl-7-(4-(4,4,5,5-tetramethyl-1,3,2-dioxaborolan-2-yl)phenyl)-2,7-diazaspiro[3.5]nonane (38.2 mg, 0.10 mmol) to give the title compound (25 mg, 60% yield). ¹H NMR (500 MHz, DMSO-d₆) δ=8.09 (br s, 1H), 7.59 (dd, J=10.1, 17.6 Hz, 2H), 7.42 (d, J=7.0 Hz, 1H), 7.36 (br d, J=8.3 Hz, 2H), 6.96 (d, J=8.8 Hz, 2H), 6.15 (s, 2H), 3.40 (br dd, J=4.0, 6.1 Hz, 2H), 3.17-3.05 (m, 5H), 2.91 (br s, 6H), 1.93-1.83 (m, 2H), 1.81-1.70 (m, 6H), 1.60 (s, 2H); LCMS: [M+H]⁺=530.44.

### Example 395: 6-(2-amino-6-fluoro-5-(4-(4-isopropylpiperazin-1-yl)phenyl)pyridin-3-yl)-4,4-difluoro-3-methoxy-3-methyl-3,4-dihydroisoquinolin-1(2H)-one (I-450)

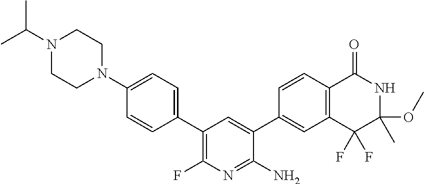 [SMILES: COC1(C)NC(=O)c2ccc(-c3cc(-c4ccc(N5CCN(C(C)C)CC5)cc4)c(F)nc3N)cc2C1(F)F]

Step 1: 6-bromo-4,4-difluoro-3-methoxy-3-methyl-3,4-dihydroisoquinolin-1(2H)-one

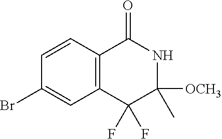 [SMILES: COC1(C)NC(=O)c2ccc(Br)cc2C1(F)F]

A vial was charged with 6-bromo-3-methyl-2H-isoquinolin-1-one (300 mg, 1.260 mmol) and Selectfluor™ fluorinating reagent >95% in F+active (536 mg, 1.512 mmol). Methanol (6 ml) and acetonitrile (6 ml) were added, and the reaction was heated at 50° C. overnight. The mixture was purified by flash chromatography (Biotage, silica gel) eluting with 0-70% EtOAc/Hexanes. The desired fractions were collected, concentrated and dried under vacuum to give the product (81 mg, 21% yield) as a white solid. ¹H NMR (500 MHz, DMSO-d₆) δ ppm 9.43 (br. s., 1H), 7.91-7.95 (m, 2H), 7.86-7.89 (m, 1H), 3.13 (s, 3H), 1.54 (d, J=0.98 Hz, 3H); LCMS: [M+H]⁺=306.11.

Step 2: (4,4-difluoro-3-methoxy-3-methyl-1-oxo-1,2,3,4-tetrahydroisoquinolin-6-yl)boronic acid

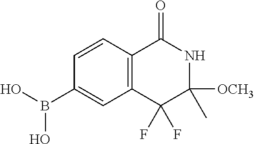 [SMILES: COC1(C)NC(=O)c2ccc(B(O)O)cc2C1(F)F]

Prepared from 6-bromo-4,4-difluoro-3-methoxy-3-methyl-3,4-dihydroisoquinolin-1(2H)-one (40 mg, 0.131 mmol) to give a mixture of boronate and boronic acid which was used in the next step without further purification. LCMS: [M+H]⁺=272.23 Boronic acid, 354.29 Boronate.

Step 3: 6-(2-amino-6-fluoro-5-(4-(4-isopropylpiperazin-1-yl)phenyl)pyridin-3-yl)-4,4-difluoro-3-methoxy-3-methyl-3,4-dihydroisoquinolin-1(2H)-one

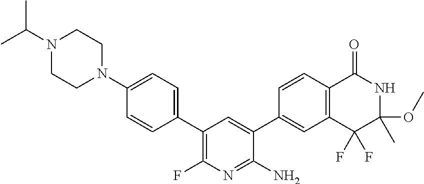 [SMILES: COC1(C)NC(=O)c2ccc(-c3cc(-c4ccc(N5CCN(C(C)C)CC5)cc4)c(F)nc3N)cc2C1(F)F]

Prepared from (4,4-difluoro-3-methoxy-3-methyl-1-oxo-1,2,3,4-tetrahydroisoquinolin-6-yl)boronic acid (34.7 mg, 0.128 mmol) and 3-bromo-6-fluoro-5-(4-(4-isopropylpiperazin-1-yl)phenyl)pyridin-2-amine (42 mg, 0.107 mmol) to give the title compound (39.3 mg, 68.2% yield) as a yellow solid. ¹H NMR (500 MHz, DMSO-d₆) δ ppm 9.32 (d, J=2.69 Hz, 1H), 8.01 (d, J=7.95 Hz, 1H), 7.83 (d, J=8.07 Hz, 1H), 7.81 (s, 1H), 7.64 (d, J=10.03 Hz, 1H), 7.42 (d, J=7.95 Hz, 2H), 6.96 (d, J=8.80 Hz, 2H), 6.25 (s, 2H), 3.15 (s, 3H), 3.12-3.15 (m, 4H), 2.68 (br. s., 1H), 2.58 (br. s., 4H), 1.56 (s, 3H), 1.01 (d, J=6.48 Hz, 6H); LCMS [M+H]⁺ 540.35.

### Example 396: 6-(2-amino-5-(4-(4-cyclopentylpiperazin-1-yl)-3-((dimethylamino)methyl)phenyl)-6-fluoropyridin-3-yl)-3,4-dihydroisoquinolin-1(2H)-one (1-451)

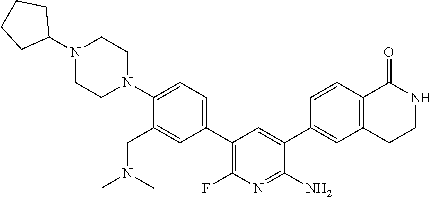 [SMILES: CN(C)Cc1cc(-c2cc(-c3ccc4c(c3)CCNC4=O)c(N)nc2F)ccc1N1CCN(C2CCCC2)CC1]

Step 1: 5-bromo-2-(4-cyclopentylpiperazin-1-yl)benzaldehyde

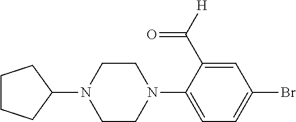 [SMILES: O=Cc1cc(Br)ccc1N1CCN(C2CCCC2)CC1]

Prepared from 5-bromo-2-fluorobenzaldehyde (0.8 g, 3.94 mmol) and 1-cyclopentylpiperazine (0.912 g, 5.91 mmol) to give the product as a pale-yellow solid (881 mg, 66% yield). LCMS: [M+H]⁺=337.26 Step 2: 1-(5-bromo-2-(4-cyclopentylpiperazin-1-yl)phenyl)-N,N-dimethylmethanamine

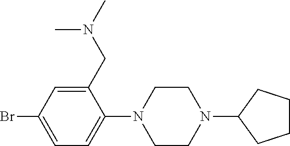 [SMILES: CN(C)Cc1cc(Br)ccc1N1CCN(C2CCCC2)CC1]

Prepared from 5-bromo-2-(4-cyclopentylpiperazin-1-yl)benzaldehyde (423 mg, 1.254 mmol) and dimethylamine, 2.0M solution in THF (5.02 ml, 10.03 mmol) as the amine to give the product was isolated as a pale-yellow solid (436 mg, 95% yield). LCMS: [M +H]⁺=366.36

Step 3: 6-(2-amino-5-(4-(4-cyclopentylpiperazin-1-yl)-3-((dimethylamino)methyl)phenyl)-6-fluoropyridin-3-yl)-3,4-dihydroisoquinolin-1(2H)-one

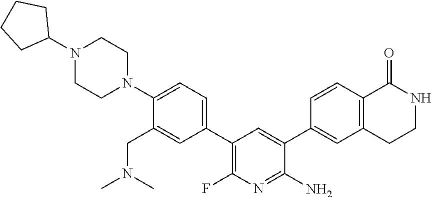 [SMILES: CN(C)Cc1cc(-c2cc(-c3ccc4c(c3)CCNC4=O)c(N)nc2F)ccc1N1CCN(C2CCCC2)CC1]

Prepared from 1-(5-bromo-2-(4-cyclopentylpiperazin-1-yl)phenyl)-N,N-dimethylmethanamine (60 mg, 0.164 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (82 mg, 0.213 mmol) to yield the title compound as a beige solid (78 mg, 83% yield). ¹H NMR (500 MHz, METHANOL-d₄) δ ppm 8.06 (d, J=7.95 Hz, 1H), 7.71 (d, J=9.78 Hz, 1H), 7.60 (s, 1H), 7.52-7.55 (m, 1H), 7.53 (dd, J=8.01, 1.28 Hz, 1H), 7.45-7.50 (m, 2H), 7.21-7.26 (m, 1H), 3.61-3.66 (m, 2H), 3.57 (t, J=6.66 Hz, 2H), 3.08 (t, J=6.60 Hz, 2H), 3.00 (t, J=4.58 Hz, 4 H), 2.67-2.88 (m, 4H), 2.58-2.65 (m, 1H), 2.29 (s, 6H), 1.98 (d, J=5.99 Hz, 2H), 1.73-1.81 (m, 2H), 1.61-1.68 (m, 2H), 1.45-1.53 (m, 2H); LCMS: [M+H]⁺=543.54

### Example 397: 6-(2-amino-5-(4-(4-cyclopentylpiperazin-1-yl)-3-((dimethylamino)methyl)phenyl)-6-fluoropyridin-3-yl)-7-fluoro-3,4-dihydroisoquinolin-1(2H)-one (I-452)

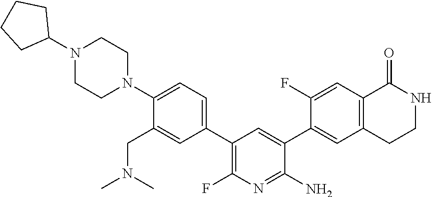 [SMILES: CN(C)Cc1cc(-c2cc(-c3cc4c(cc3F)C(=O)NCC4)c(N)nc2F)ccc1N1CCN(C2CCCC2)CC1]

Prepared from 1-(5-bromo-2-(4-cyclopentylpiperazin-1-yl)phenyl)-N,N-dimethylmethanamine (60 mg, 0.164 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (85 mg, 0.213 mmol) to give the title compound as a white solid (36 mg, 37% yield). 1H NMR (500 MHz, METHANOL-d4) δ ppm 7.62-7.67 (m, 1H), 7.58 (d, J=9.78 Hz, 1H), 7.47 (s, 1H), 7.35 (d, J=8.31 Hz, 1H), 7.30 (d, J=6.72 Hz, 1H), 7.12 (d, J=8.44 Hz, 1H), 3.53 (s, 2H), 3.44 (t, J=6.66 Hz, 2H), 2.92 (t, J=6.60 Hz, 2H), 2.88 (t, J=4.52 Hz, 4H), 2.55-2.76 (m, 4 H), 2.45-2.54 (m, 1H), 2.18 (s, 6H), 1.83-1.90 (m, 2H), 1.59-1.68 (m, 2H), 1.48-1.56 (m, 2H), 1.33-1.42 (m, 2H); LCMS: [M+H]⁺=561.53

### Example 398: 6-(2-amino-5-(4-(4-(4,4-difluorobutyl)piperazin-1-yl)-3-((dimethylamino)methyl)phenyl)-6-fluoropyridin-3-yl)isoquinolin-1(2H)-one (I-453)

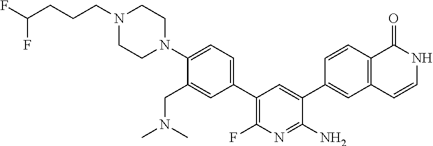 [SMILES: CN(C)Cc1cc(-c2cc(-c3ccc4c(=O)[nH]ccc4c3)c(N)nc2F)ccc1N1CCN(CCCC(F)F)CC1]

Step 1: -butyl 4-(2-((dimethylamino)methyl)-4-(4,4,5,5-tetramethyl-1,3,2-dioxaborolan-2-yl)phenyl)piperazine-1-carboxylate

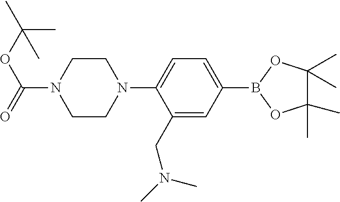 [SMILES: CN(C)Cc1cc(B2OC(C)(C)C(C)(C)O2)ccc1N1CCN(C(=O)OC(C)(C)C)CC1]

Prepared from tert-butyl 4-(4-bromo-2-((dimethylamino)methyl)phenyl)piperazine-1-carboxylate (80 mg, 0.201 mmol) using similar procedures as described previously to afford the product as a black solid (89 mg, 97% yield) which was taken to the next step without any purification. LCMS: [M+H]⁺=446.40 Step 2: tert-butyl 4-(4-(6-amino-2-fluoro-5-(1-oxo-1,2-dihydroisoquinolin-6-yl)pyridin-3-yl)-2-((dimethylamino)methyl)phenyl)piperazine-1-carboxylate

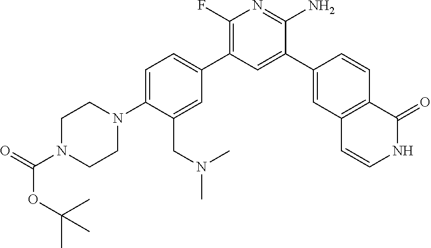 [SMILES: CN(C)Cc1cc(-c2cc(-c3ccc4c(=O)[nH]ccc4c3)c(N)nc2F)ccc1N1CCN(C(=O)OC(C)(C)C)CC1]

Prepared from tert-butyl 4-(2-((dimethylamino)methyl)-4-(4,4,5,5-tetramethyl-1,3,2-dioxaborolan-2-yl)phenyl)piperazine-1-carboxylate (85 mg, 0.185 mmol) and 6-(2-amino-5-bromo-6-fluoropyridin-3-yl)isoquinolin-1(2H)-one (49.5 mg, 0.148 mmol) to give the product as a brown solid (25 mg, 23% yield). LCMS: [M+H]⁺=573.44

Step 3: 6-(2-amino-5-(3-((dimethylamino)methyl)-4-(piperazin-1-yl)phenyl)-6-fluoropyridin-3-yl)isoquinolin-1(2H)-one

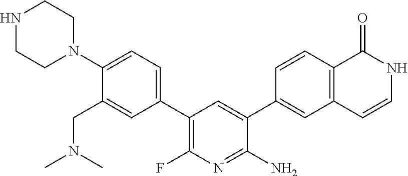 [SMILES: CN(C)Cc1cc(-c2cc(-c3ccc4c(=O)[nH]ccc4c3)c(N)nc2F)ccc1N1CCNCC1]

Prepared from tert-butyl 4-(4-(6-amino-2-fluoro-5-(1-oxo-1,2-dihydroisoquinolin-6-yl)pyridin-3-yl)-2-((dimethylamino)methyl)phenyl)piperazine-1-carboxylate (25 mg, 0.034 mmol) with TFA (0.259 mL, 3.38 mmol) to give the title compound as a brown solid (12 mg, 75% yield); LCMS: [M+H]⁺=475.34.

Step 4: 6-(2-amino-5-(4-(4-(4,4-difluorobutyl)piperazin-1-yl)-3-((dimethylamino)methyl)phenyl)-6-fluoropyridin-3-yl)isoquinolin-1(2H)-one

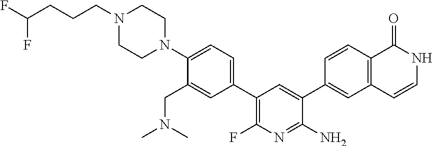 [SMILES: CN(C)Cc1cc(-c2cc(-c3ccc4c(=O)[nH]ccc4c3)c(N)nc2F)ccc1N1CCN(CCCC(F)F)CC1]

Prepared from 6-(2-amino-5-(3-((dimethylamino)methyl)-4-(piperazin-1-yl)phenyl)-6-fluoropyridin-3-yl)isoquinolin-1(2H)-one (12 mg, 0.025 mmol) and 4-bromo-1,1-difluorobutane (8.79 mg, 0.051 mmol) to give the title compound as a beige solid (6 mg, 38% yield). 1H NMR (500 MHz, METHANOL-d₄) δ ppm 8.42 (d, J=8.31 Hz, 1H), 7.82 (s, 1H), 7.77 (d, J=9.66 Hz, 1H), 7.68 (d, J=8.31 Hz, 1H), 7.63 (s, 1H), 7.53-7.57 (m, 1H), 7.55 (d, J=8.31 Hz, 1H), 7.27-7.35 (m, 1H), 7.19-7.27 (m, 1H), 6.66-6.80 (m, 1H), 5.84-6.10 (m, 1H), 3.72-3.88 (m, 2H), 3.01 (t, J=4.46 Hz, 4H), 2.71 (br. s., 4H), 2.54 (d, J=7.34 Hz, 2H), 2.43 (s, 6H), 1.87-1.96 (m, 2H), 1.71-1.78 (m, 2H); LCMS: [M+H]⁺=565.54.

### Example 399: 6-(2-amino-5-(4-(4-(cyclopropylmethyl)piperazin-1-yl)phenyl)-6-fluoropyridin-3-yl)-8-fluoro-3-methylisoquinolin-1(2H)-one (I-454)

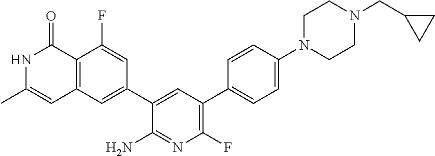 [SMILES: Cc1cc2cc(-c3cc(-c4ccc(N5CCN(CC6CC6)CC5)cc4)c(F)nc3N)cc(F)c2c(=O)[nH]1]

Step 1: 6-bromo-8-fluoro-3-methylisoquinolin-1(2H)-one

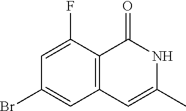

To an RBF was added copper(I) bromide (0.209 g, 1.455 mmol), cesium carbonate (9.48 g, 29.1 mmol), 2,4-dibromo-6-fluorobenzamide (4.32 g, 14.55 mmol), propan-2-one (5.34 ml, 72.7 mmol) and dimethylsulfoxide (DMSO) (150 ml). The reaction was stirred and heated at 80° C. overnight. The reaction mixture was partitioned between brine (200 ml) and DCM (200 ml). The organic layer was separated, and the aqueous layer washed with DCM (2×100 ml). The combined organic layers were washed with brine, dried over anhydrous Na₂SO₄, and concentrated onto celite. The mixture was purified by flash chromatography (Biotage, silica gel) eluting with 0-100% EtOAc/Hexanes. The desired fractions were collected, concentrated and dried under vacuum to afford the product (2.41 g, 64.7% yield) as a pale-yellow solid. ¹H NMR (500 MHz, DMSO-d₆) δ ppm 11.39 (br. s., 1H), 7.62 (s, 1H), 7.38 (d, J=11.00 Hz, 1H), 6.30 (s, 1H), 2.18 (s, 3H); LCMS: [M+H]⁺=256.08.

Step 2: (8-fluoro-3-methyl-1-oxo-1,2-dihydroisoquinolin-6-yl)boronic acid

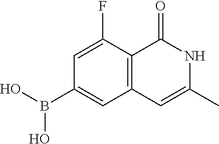 [SMILES: Cc1cc2cc(B(O)O)cc(F)c2c(=O)[nH]1]

Prepared from 6-bromo-8-fluoro-3-methylisoquinolin-1(2H)-one (31 mg, 0.121 mmol) to give the boronic acid intermediate. LCMS: [M+H]⁺=222.35.

Step 3: 6-(2-amino-5-(4-(4-(cyclopropylmethyl)piperazin-1-yl)phenyl)-6-fluoropyridin-3-yl)-8-fluoro-3-methylisoquinolin-1(2H)-one

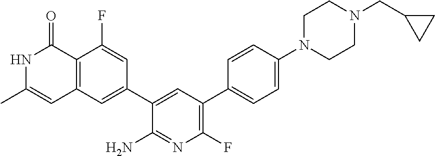 [SMILES: Cc1cc2cc(-c3cc(-c4ccc(N5CCN(CC6CC6)CC5)cc4)c(F)nc3N)cc(F)c2c(=O)[nH]1]

Prepared from (8-fluoro-3-methyl-1-oxo-1,2-dihydroisoquinolin-6-yl)boronic acid (31.6 mg, 0.143 mmol) and 3-chloro-5-(4-(4-(cyclopropylmethyl)piperazin-1-yl)phenyl)-6-fluoropyridin-2-amine (43 mg, 0.119 mmol) to give the title compound (16.2 mg, 27.1% yield) as a pale yellow solid. ¹H NMR (500 MHz, DMSO-d₆) δ ppm 11.24 (s, 1H), 7.65 (d, J=10.15 Hz, 1H), 7.44 (s, 1H), 7.42 (d, J=8.19 Hz, 2H), 7.22 (d, J=12.47 Hz, 1H), 6.98 (d, J=8.44 Hz, 2H), 6.34 (s, 1H), 6.29 (br. s., 2H), 3.17 (br. s., 4H), 2.59 (br. s., 4H), 2.23 (br. s., 2H), 2.19 (s, 3H), 0.81-0.94 (m, 1H), 0.42-0.61 (m, 2H), 0.04-0.23 (m, 2H); LCMS: [M+H]⁺=502.37.

### Example 400: 6-(2-amino-5-(4-(4-(cyclopropylmethyl)piperazin-1-yl)phenyl)-6-fluoropyridin-3-yl)-4-fluoroisoquinolin-1(2H)-one (I-455)

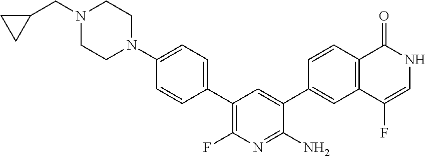 [SMILES: Nc1nc(F)c(-c2ccc(N3CCN(CC4CC4)CC3)cc2)cc1-c1ccc2c(=O)[nH]cc(F)c2c1]

Step 1: 6-bromo-4-fluoroisoquinolin-1(2H)-one

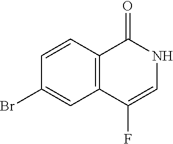 [SMILES: O=c1[nH]cc(F)c2cc(Br)ccc12]

A vial was charged with 6-bromo-2H-isoquinolin-1-one (1000 mg, 4.46 mmol) and Selectfluor™ (1897 mg, 5.36 mmol). Methanol (10 ml) and acetonitrile (10 ml) were added, and the reaction was heated to 50° C. for 1 h. The reaction was concentrated in vacuo, dissolved in 1,2-dichloroethane (20 ml), and phosphorous (V) oxychloride (0.835 ml, 8.93 mmol) was added. The reaction was stirred at 50° C. for 1 h. The mixture was poured into a RBF and concentrated in vacuo to remove the volatiles then the aqueous suspension was filtered, washing with water. The collected solid was dried under vacuum to give the product (1.1 g, quantitative) as a light pink solid. The material was used in the next step as-is. LCMS: [M+H]⁺=242.14.

Step 2: (4-fluoro-1-oxo-1,2-dihydroisoquinolin-6-yl)boronic acid

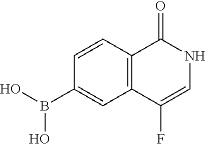 [SMILES: O=c1[nH]cc(F)c2cc(B(O)O)ccc12]

The title compound was prepared from 6-bromo-4-fluoroisoquinolin-1(2H)-one (920 mg, 3.80 mmol) to give a mixture of boronate and boronic acid which was used in the next step without further purification. LCMS: [M+H]⁺=290.31 Boronate, 208.21 Boronic acid.

Step 3: 6-(2-amino-5-bromo-6-fluoropyridin-3-yl)-4-methylisoquinolin-1(2H)-one

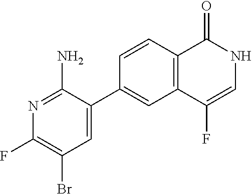 [SMILES: Nc1nc(F)c(Br)cc1-c1ccc2c(=O)[nH]cc(F)c2c1]

Prepared from 5-bromo-6-fluoro-3-iodopyridin-2-amine (500 mg, 1.578 mmol) and (4-fluoro-1-oxo-1,2-dihydroisoquinolin-6-yl)boronic acid (392 mg, 1.893 mmol) to give the product (544 mg, 88% yield) as a beige solid. LCMS [M+H]⁺=352.19.

Step 4: 1-(4-bromophenyl)-4-(cyclopropylmethyl)piperazine

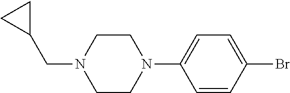 [SMILES: Brc1ccc(N2CCN(CC3CC3)CC2)cc1]

To a solution of 1-(4-bromophenyl)piperazine (1781 mg, 7.39 mmol) and N,N-diisopropylethylamine (6.43 ml, 36.9 mmol) in N,N-dimethylformamide (10 ml) was added (bromomethyl)cyclopropane (1.433 ml, 14.77 mmol) then stirred at RT overnight. The mixture was diluted with water and extracted with DCM (3x). The combined organic layers were washed with brine, dried over anhydrous MgSO₄ and concentrated onto celite. The crude material was purified by flash chromatography (Biotage, silica gel) eluting with 0-100% EtOAc/Hexanes. The desired fractions were collected, concentrated and dried under vacuum to afford the product (2038 mg, 93% yield) as an off-white solid. LCMS[M+H]⁺ 295.37.

Step 5: 1-(cyclopropylmethyl)-4-(4-(4,4,5,5-tetramethyl-1,3,2-dioxaborolan-2-yl)phenyl)piperazine

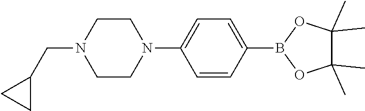 [SMILES: CC1(C)OB(c2ccc(N3CCN(CC4CC4)CC3)cc2)OC1(C)C]

Prepared from 1-(4-bromophenyl)-4-(cyclopropylmethyl)piperazine (1.88 g, 6.37 mmol) to give the boronate which was used in the next step without further purification. LCMS: [M+H]⁺=343.58.

Step 6: 6-(2-amino-5-(4-(4-(cyclopropylmethyl)piperazin-1-yl)phenyl)-6-fluoropyridin-3-yl)-4-fluoroisoquinolin-1(2H)-one

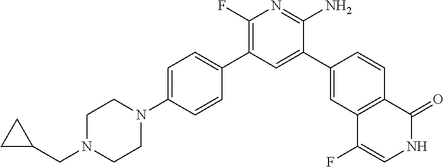 [SMILES: Nc1nc(F)c(-c2ccc(N3CCN(CC4CC4)CC3)cc2)cc1-c1ccc2c(=O)[nH]cc(F)c2c1]

Prepared from 6-(2-amino-5-bromo-6-fluoropyridin-3-yl)-4-fluoroisoquinolin-1(2H)-one (30 mg, 0.085 mmol) and 1-(cyclopropylmethyl)-4-(4-(4,4,5,5-tetramethyl-1,3,2-dioxaborolan-2-yl)phenyl)piperazine (35.0 mg, 0.102 mmol) to give the title compound (10.1 mg, 24% yield) as a beige solid. ¹H NMR (500 MHz, DMSO-d₆) δ ppm 11.15 (d, J=4.89 Hz, 1H), 8.27 (dd, J=8.31, 1.47 Hz, 1H), 7.82 (s, 1H), 7.73 (d, J=8.31 Hz, 1H), 7.67 (d, J=10.15 Hz, 1H), 7.43 (br. s., 1H), 7.41 (s, 2H), 6.98 (d, J=8.56 Hz, 2H), 6.28 (br. s., 2H), 3.17 (br. s., 4H), 2.58 (br. s., 4H), 2.23 (br. s., 2H), 0.87 (br. s., 1H), 0.49 (d, J=5.50 Hz, 2H), 0.11 (br. s., 2H); LCMS: [M+H]⁺ 488.39.

### Example 401: 6-(2-amino-5-(4-(4-(cyclopropylmethyl)piperazin-1-yl)phenyl)-6-fluoropyridin-3-yl)-4-fluoro-3-methylisoquinolin-1(2H)-one (I-456)

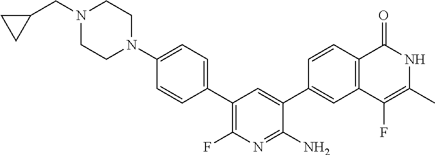 [SMILES: Cc1[nH]c(=O)c2ccc(-c3cc(-c4ccc(N5CCN(CC6CC6)CC5)cc4)c(F)nc3N)cc2c1F]

Step 1: 4-fluoro-3-methyl-6-(4,4,5,5-tetramethyl-1,3,2-dioxaborolan-2-yl)isoquinolin-1(2H)-one

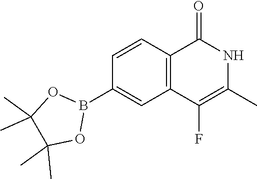

Prepared from 6-bromo-4-fluoro-3-methylisoquinolin-1(2H)-one (72 mg, 0.281 mmol) to give a mixture of boronate and boronic acid which was used in the next step without further purification. LCMS: [M+H]⁺=222.23 Boronic acid, 304.29 Boronate.

Step 2: 3-chloro-5-(4-(4-(cyclopropylmethyl)piperazin-1-yl)phenyl)-6-fluoropyridin-2-amine

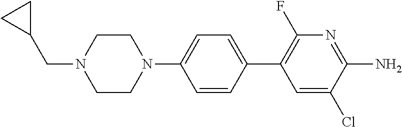 [SMILES: Nc1nc(F)c(-c2ccc(N3CCN(CC4CC4)CC3)cc2)cc1Cl]

To a vial was added 5-bromo-3-chloro-6-fluoropyridin-2-amine (1200 mg, 5.32 mmol), 1-(cyclopropylmethyl)-4-(4-(4,4,5,5-tetramethyl-1,3,2-dioxaborolan-2-yl)phenyl)piperazine (2186 mg, 6.39 mmol), [1,1′-bis(diphenylphosphino)ferrocene]dichloropalladium(II) (389 mg, 0.532 mmol), Cs₂CO₃ (4336 mg, 13.31 mmol), 1,2-dimethoxyethane (12 mL) and water (3 mL) (4:1 DME/H₂O). The vial was flushed with nitrogen, then heated at 90° C. for 7 hours. The reaction was concentrated onto celite and purified by flash chromatography (Biotage, silica gel) eluting with 0-10% MeOH/DCM+1% NH₄OH, followed by flash chromatography eluting with 0-100% EtOAc/Hexanes. The desired fractions were collected, concentrated and dried under vacuum afford the product (1392 mg, 72.5% yield) as a beige solid. LCMS: [M+H]⁺=361.50.

Step 3: 6-(2-amino-5-(4-(4-(cyclopropylmethyl)piperazin-1-yl)phenyl)-6-fluoropyridin-3-yl)-4-fluoro-3-methylisoquinolin-1(2H)-one

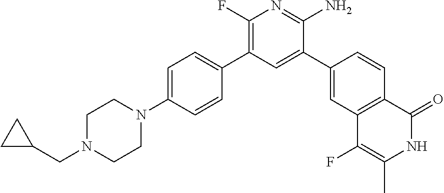 [SMILES: Cc1[nH]c(=O)c2ccc(-c3cc(-c4ccc(N5CCN(CC6CC6)CC5)cc4)c(F)nc3N)cc2c1F]

Prepared from 4-fluoro-3-methyl-6-(4,4,5,5-tetramethyl-1,3,2-dioxaborolan-2-yl)isoquinolin-1(2H)-one (40.7 mg, 0.134 mmol) and 3-chloro-5-(4-(4-(cyclopropylmethyl)piperazin-1-yl)phenyl)-6-fluoropyridin-2-amine (44 mg, 0.122 mmol) to give the title compound (15.8 mg, 25.8% yield) as a white solid. ¹H NMR (500 MHz, DMSO-d₆) δ ppm 11.25 (br. s., 1H), 8.18-8.24 (m, 1H), 7.75 (s, 1H), 7.62-7.68 (m, 2H), 7.42 (d, J=8.19 Hz, 2H), 6.99 (d, J=8.19 Hz, 2H), 6.26 (br. s., 2H), 3.17 (br. s., 4H), 2.62 (d, J=19.44 Hz, 2H), 2.24 (d, J=2.81 Hz, 3H), 0.88 (br. s., 1H), 0.50 (br. s., 2H), 0.12 (br. s., 2H); LCMS: [M+H]⁺=502.49.

### Example 402: 6-(2-amino-5-(4-(4-(cyclopropylmethyl)piperazin-1-yl)phenyl)-6-fluoropyridin-3-yl)-4-methylisoquinolin-1(2H)-one (I-457)

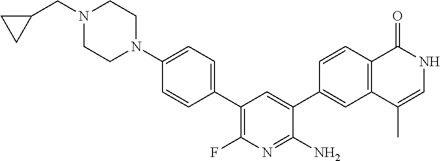 [SMILES: Cc1c[nH]c(=O)c2ccc(-c3cc(-c4ccc(N5CCN(CC6CC6)CC5)cc4)c(F)nc3N)cc12]

Prepared from 6-(2-amino-5-bromo-6-fluoropyridin-3-yl)-4-methylisoquinolin-1(2H)-one (40 mg, 0.115 mmol) and 1-(cyclopropylmethyl)-4-(4-(4,4,5,5-tetramethyl-1,3,2-dioxaborolan-2-yl)phenyl)piperazine (47.2 mg, 0.138 mmol) to give the title compound (11.4 mg, 20.5% yield) as a yellow solid. ¹H NMR (500 MHz, DMSO-d₆) δ ppm 11.08 (d, J=5.26 Hz, 1H), 8.28 (d, J=8.31 Hz, 1H), 7.72 (s, 1H), 7.66 (d, J=10.15 Hz, 1H), 7.62 (d, J=8.19 Hz, 1H), 7.41 (d, J=8.19 Hz, 2H), 7.04 (d, J=5.26 Hz, 1H), 6.98 (d, J=8.68 Hz, 2H), 6.22 (s, 2H), 3.17 (br. s., 4H), 2.59 (br. s., 4H), 2.24 (s, 5H), 0.86 (d, J=5.62 Hz, 1H), 0.46-0.51 (m, 2H), 0.06-0.13 (m, 2H); LCMS [M+H]⁺ 484.32.

### Example 403: 2-(2-amino-6-fluoro-5-(4-(4-isopropylpiperazin-1-yl)phenyl)pyridin-3-yl)-7,8-dihydropyrido[4,3-d]pyrimidin-5(6H)-one (I-458)

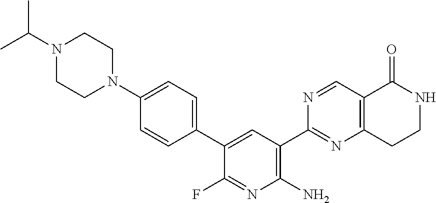 [SMILES: CC(C)N1CCN(c2ccc(-c3cc(-c4ncc5c(n4)CCNC5=O)c(N)nc3F)cc2)CC1]

Step 1: tert-butyl 2-chloro-5-oxo-7,8-dihydropyrido[4,3-d]pyrimidine-6(5H)-carboxylate

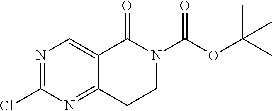 [SMILES: CC(C)(C)OC(=O)N1CCc2nc(Cl)ncc2C1=O]

To a solution of tert-butyl 2-chloro-7,8-dihydropyrido[4,3-d]pyrimidine-6(5H)-carboxylate (500 mg, 1.854 mmol), ruthenium(III) chloride hydrate 99.98% (125 mg, 0.556 mmol), sodium (meta)periodate (1189 mg, 5.56 mmol), CH₂Cl₂ (2 mL), and water (2 mL) was added 2 drops of acetonitrile. The reaction was stirred at RT overnight. The reaction was quenched with isopropanol (1 mL), filtered over celite and the filter cake was washed with DCM (20 mL). Water was added and the aqueous layer was extracted with CH₂Cl₂ (2×20 mL), and the combined organics were dried over anhydrous Na₂SO₄, then filtered and concentrated. The residue was purified by flash chromatography over silica gel (0-50% EtOAc/hexane eluent) to give the product (399 mg, 76% yield) as an off-white solid. LCMS: [M+H-tBu]⁺=228.18.

Step 2: tert-butyl2-(2-amino-6-fluoro-5-(4-(4-isopropylpiperazin-1-yl)phenyl)pyridin-3-yl)-5-oxo-7,8-dihydropyrido[4, 3-d]pyrimidine-6(5H)-carboxylate

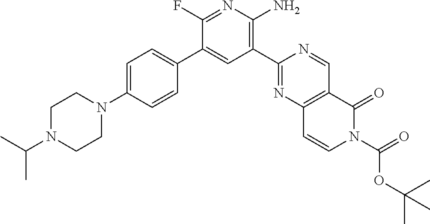 [SMILES: CC(C)N1CCN(c2ccc(-c3cc(-c4ncc5c(=O)n(C(=O)OC(C)(C)C)ccc5n4)c(N)nc3F)cc2)CC1]

Prepared from tert-butyl 2-chloro-5-oxo-7,8-dihydropyrido[4,3-d]pyrimidine-6(5H)-carboxylate (69.6 mg, 0.245 mmol) and 6-fluoro-5-(4-(4-isopropylpiperazin-1-yl)phenyl)-3-(4,4,5,5-tetramethyl-1,3,2-dioxaborolan-2-yl)pyridin-2-amine (90 mg, 0.204 mmol) to give the product (50.2 mg, 44% yield) as a yellow solid. LCMS: [M+H]⁺=562.34.

Step 3: 2-(2-amino-6-fluoro-5-(4-(4-isopropylpiperazin-1-yl)phenyl)pyridin-3-yl)-7,8-dihydropyrido[4,3-d]pyrimidin-5(6H)-one, trifluoroacetate

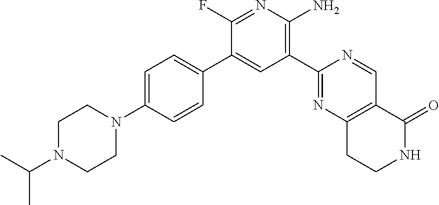 [SMILES: CC(C)N1CCN(c2ccc(-c3cc(-c4ncc5c(n4)CCNC5=O)c(N)nc3F)cc2)CC1]

Prepared from 2-(2-amino-6-fluoro-5-(4-(4-isopropylpiperazin-1-yl)phenyl)pyridin-3-yl)-5-oxo-7,8-dihydropyrido[4,3-d]pyrimidine-6(5H)-carboxylate (50.2 mg, 0.089 mmol) and TFA (0.137 mL, 1.788 mmol) to give the product (47.4 mg, 92% yield) as an orange solid. ¹H NMR (500 MHz, DMSO-d₆) δ ppm 9.31 (br. s., 1H), 9.11 (s, 1H), 8.90 (d, J=10.52 Hz, 1H), 8.23 (br. s., 1H), 7.46 (d, J=8.07 Hz, 2H), 7.12 (d, J=8.80 Hz, 2H), 3.95 (d, J=13.20 Hz, 2H), 3.52-3.60 (m, 5H), 3.18-3.24 (m, 2H), 3.12-3.17 (m, 2H), 3.01 (t, J=12.23 Hz, 2H), 1.31 (d, J=6.60 Hz, 6H); LCMS: [M+H]⁺=462.32.

### Example 404: 6-(2-amino-5-(3-((dimethylamino)methyl)-4-methoxyphenyl)-6-fluoropyridin-3-yl)-3,4-dihydroisoquinolin-1(2H)-one (I-459)

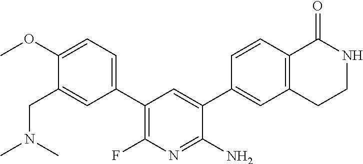 [SMILES: COc1ccc(-c2cc(-c3ccc4c(c3)CCNC4=O)c(N)nc2F)cc1CN(C)C]

Prepared from 1-(5-bromo-2-methoxyphenyl)-N,N-dimethylmethanamine (50 mg, 0.205 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (102 mg, 0.266 mmol) afford the title compound as a peach colored solid (62 mg, 68% yield). ¹H NMR ((500 MHz, METHANOL-d₄) δ ppm 8.05 (d, J=7.95 Hz, 1H), 7.65-7.70 (m, 1H), 7.52 (dd, J=8.07, 0.98 Hz, 1H), 7.50 (s, 1H), 7.43-7.48 (m, 2H), 7.47 (d, J=5.87 Hz, 2H), 7.06 (d, J=8.56 Hz, 1H), 3.83-3.92 (m, 3H), 3.58-3.62 (m, 2H), 3.56 (t, J=6.66 Hz, 2H), 3.07 (t, J=6.60 Hz, 2H), 2.30 (s, 6H); LCMS: [M+H]⁺=421.33.

### Example 405: 6-(2-amino-5-(3-((dimethylamino)methyl)-4-methoxyphenyl)-6-fluoropyridin-3-yl)-7-fluoro-3,4-dihydroisoquinolin-1(2H)-one (I-460)

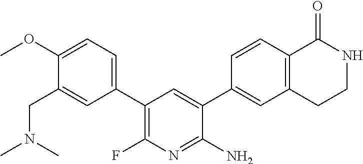 [SMILES: COc1ccc(-c2cc(-c3ccc4c(c3)CCNC4=O)c(N)nc2F)cc1CN(C)C]

Prepared from 1-(5-bromo-2-methoxyphenyl)-N,N-dimethylmethanamine (60 mg, 0.246 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (128 mg, 0.319 mmol) to afford the title compound as a white solid (36 mg, 32% yield). ¹H NMR ((500 MHz, METHANOL-d₄) δ ppm 7.76 (d, J=10.03 Hz, 1H), 7.66 (d, J=9.90 Hz, 1H), 7.48 (d, J=8.68 Hz, 1H), 7.45 (s, 1 H), 7.41-7.44 (m, 1H), 7.42 (d, J=6.85 Hz, 1H), 7.07 (d, J=8.44 Hz, 1H), 3.89 (s, 3H), 3.62 (s, 2H), 3.56 (t, J=6.66 Hz, 2H), 3.04 (t, J=6.60 Hz, 2H), 2.31 (s, 6H); LCMS: [M+H]+=432.32.

### Example 406: 6-(2-amino-5-(4-(4-cyclobutylpiperazin-1-yl)-3-((dimethylamino)methyl)phenyl)-6-fluoropyridin-3-yl)-3,4-dihydroisoquinolin-1(2H)-one (I-461)

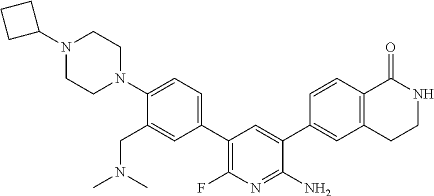 [SMILES: CN(C)Cc1cc(-c2cc(-c3ccc4c(c3)CCNC4=O)c(N)nc2F)ccc1N1CCN(C2CCC2)CC1]

Step 1: 5-bromo-2-(4-cyclobutylpiperazin-1-yl)benzaldehyde

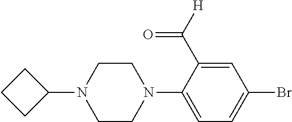 [SMILES: O=Cc1cc(Br)ccc1N1CCN(C2CCC2)CC1]

Prepared from 5-bromo-2-fluorobenzaldehyde (0.45 g, 2.217 mmol) and 1-cyclobutylpiperazine (0.497 g, 3.55 mmol). The title compound was isolated as a pale-yellow solid (241 mg, 34% yield). LCMS: [M+H]⁺=323.29 Step 2: 1-(5-bromo-2-(4-cyclobutylpiperazin-1-yl)phenyl)-N,N-dimethylmethanamine

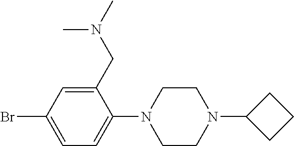 [SMILES: CN(C)Cc1cc(Br)ccc1N1CCN(C2CCC2)CC1]

Prepared from 5-bromo-2-(4-cyclobutylpiperazin-1-yl)benzaldehyde (241 mg, 0.746 mmol) and dimethylamine, 2.0M solution in THF (1.491 ml, 2.98 mmol) as the amine to give the product as an off white solid (251 mg, 96% yield). LCMS: [M+H]⁺=352.31

Step 3: 6-(2-amino-5-(4-(4-cyclobutylpiperazin-1-yl)-3-((dimethylamino)methyl)phenyl)-6-fluoropyridin-3-yl)-3,4-dihydroisoquinolin-1(2H)-one

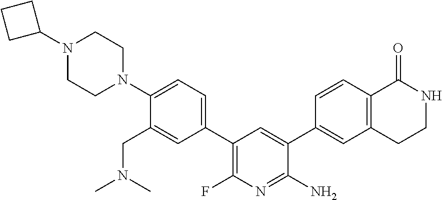 [SMILES: CN(C)Cc1cc(-c2cc(-c3ccc4c(c3)CCNC4=O)c(N)nc2F)ccc1N1CCN(C2CCC2)CC1]

Prepared from 1-(5-bromo-2-(4-cyclobutylpiperazin-1-yl)phenyl)-N,N-dimethylmethanamine (50 mg, 0.142 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (70.7 mg, 0.184 mmol) to yield the title compound as a peach colored solid (54 mg, 68% yield). ¹H NMR (500 MHz, METHANOL-d₄) δ ppm 8.05 (d, J=7.95 Hz, 1H), 7.70 (d, J=9.66 Hz, 1H), 7.59 (s, 1H), 7.52 (d, J=7.95 Hz, 1H), 7.44-7.50 (m, 2H), 7.47 (br. s., 2H), 7.24 (d, J=8.31 Hz, 1H), 3.62 (s, 2H), 3.56 (t, J=6.60 Hz, 2H), 3.07 (t, J=6.48 Hz, 2H), 2.98 (br. s., 4H), 2.87-2.94 (m, 1H), 2.41-2.77 (m, 4H), 2.28 (s, 6H), 2.12 (d, J=2.81 Hz, 2H), 1.97 (quin, J=9.69 Hz, 2H), 1.75-1.85 (m, 2H); LCMS: [M+H]⁺=529.44.

### Example 407: 6-(2-amino-5-(4-(4-cyclobutylpiperazin-1-yl)-3-((dimethylamino)methyl)phenyl)-6-fluoropyridin-3-yl)-7-fluoro-3,4-dihydroisoquinolin-1(2H)-one (I-462)

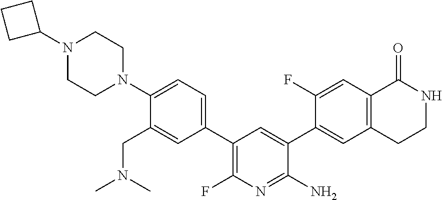 [SMILES: CN(C)Cc1cc(-c2cc(-c3cc4c(cc3F)C(=O)NCC4)c(N)nc2F)ccc1N1CCN(C2CCC2)CC1]

Prepared from 1-(5-bromo-2-(4-cyclobutylpiperazin-1-yl)phenyl)-N,N-dimethylmethanamine (50 mg, 0.142 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (74.0 mg, 0.184 mmol to yield the title compound as a white solid (36 mg, 44% yield). ¹H NMR (500 MHz, METHANOL-d₄) δ ppm 7.75 (d, J=10.03 Hz, 1H), 7.69 (d, J=9.78 Hz, 1H), 7.58 (s, 1 H), 7.45 (d, J=8.31 Hz, 1H), 7.41 (d, J=6.85 Hz, 1H), 7.23 (d, J=8.31 Hz, 1H), 3.61 (s, 2H), 3.56 (t, J=6.66 Hz, 2H), 3.03 (t, J=6.54 Hz, 2H), 2.98 (t, J=4.40 Hz, 4H), 2.90 (t, J=7.83 Hz, 1H), 2.42-2.72 (m, 4H), 2.27 (s, 6H), 2.09-2.16 (m, 2H), 1.93-2.01 (m, 2H), 1.74-1.84 (m, 2H), 1.74-1.84 (m, 2H); LCMS: [M+H]⁺=547.43

### Example 408: 6-(2-amino-6-fluoro-5-(2-methyl-1,2,3,4-tetrahydroisoquinolin-5-yl)pyridin-3-yl)-3,4-dihydroisoquinolin-1(2H)-one (I-463)

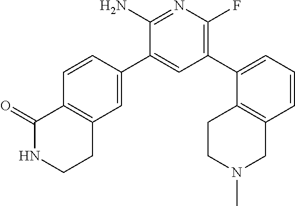 [SMILES: CN1CCc2c(cccc2-c2cc(-c3ccc4c(c3)CCNC4=O)c(N)nc2F)C1]

Step 1: 5-bromo-2-methyl-1,2,3,4-tetrahydroisoquinoline

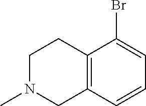

Prepared from 5-bromo-1,2,3,4-tetrahydroisoquinoline (750 mg, 3.54 mmol) and formaldehyde solution, 37% wt in water (0.790 ml, 10.61 mmol) to give the product as a colorless oil (794 mg, 99% yield). LCMS: [M+H]⁺=225.81

Step 2: 6-(2-amino-6-fluoro-5-(2-methyl-1,2,3,4-tetrahydroisoquinolin-5-yl)pyridin-3-yl)-3,4-dihydroisoquinolin-1(2H)-one

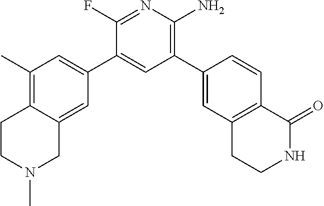 [SMILES: Cc1cc(-c2cc(-c3ccc4c(c3)CCNC4=O)c(N)nc2F)cc2c1CCN(C)C2]

Prepared as per the procedure analogous to that of Example 2, step 1, by coupling 5-bromo-2-methyl-1,2,3,4-tetrahydroisoquinoline (60 mg, 0.265 mmol) from Example 114 Step 1 with 6-(2-amino-6-fluoro-5-(4,4,5,5-tetramethyl-1,3,2-dioxaborolan-2-yl)pyridin-3-yl)-3,4-dihydroisoquinolin-1(2H)-one (132 mg, 0.345 mmol) to afford the title compound as a peach colored solid (92 mg, 82% yield). ¹H NMR (500 MHz, METHANOL-d₄) δ ppm 8.03 (d, J=7.95 Hz, 1H), 7.48 (d, J=7.83 Hz, 1H), 7.44 (s, 1H), 7.39 (d, J=9.41 Hz, 1H), 7.19-7.25 (m, 1H), 7.10 (t, J=6.97 Hz, 2H), 3.68 (br. s., 2H), 3.55 (t, J=6.54 Hz, 2H), 3.05 (t, J=6.42 Hz, 2H), 2.77 (br. s., 2H), 2.64-2.73 (m, 2H), 2.46 (s, 3H); LCMS: [M +H]⁺=403.40

### Example 409: 6-(2-amino-6-fluoro-5-(2-methyl-1,2,3,4-tetrahydroisoquinolin-5-yl)pyridin-3-yl)-7-fluoro-3,4-dihydroisoquinolin-1(2H)-one (I-464)

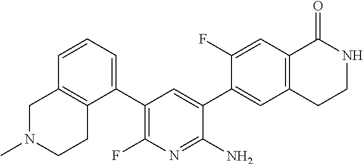 [SMILES: CN1CCc2c(cccc2-c2cc(-c3cc4c(cc3F)C(=O)NCC4)c(N)nc2F)C1]

Prepared from 5-bromo-2-methyl-1,2,3,4-tetrahydroisoquinoline (60 mg, 0.265 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (138 mg, 0.345 mmol) to afford the title compound as a white solid (49 mg, 42% yield). ¹H NMR (500 MHz, METHANOL-d₄) δ ppm 7.74 (d, J=10.03 Hz, 1H), 7.35-7.43 (m, 2H), 7.19-7.25 (m, 1H), 7.10 (t, J=7.95 Hz, 2H), 3.68 (s, 2H), 3.54 (t, J=6.66 Hz, 2H), 3.02 (t, J=6.54 Hz, 2H), 2.77 (d, J=3.79 Hz, 2H), 2.73 (d, J=5.14 Hz, 2H), 2.46 (s, 3H); LCMS: [M+H]⁺=421.39

### Example 411: 3-amino-7-(2-amino-6-fluoro-5-(4-(4-isopropylpiperazin-1-yl)phenyl)pyridin-3-yl)-2-methylquinazolin-4(3H)-one (I-465)

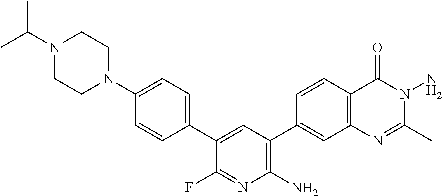 [SMILES: Cc1nc2cc(-c3cc(-c4ccc(N5CCN(C(C)C)CC5)cc4)c(F)nc3N)ccc2c(=O)n1N]

Prepared from 3-amino-7-chloro-2-methyl-3,4-dihydroquinazolin-4-one (30 mg, 0.143 mmol) and 6-fluoro-5-(4-(4-isopropylpiperazin-1-yl)phenyl)-3-(4,4,5,5-tetramethyl-1,3,2-dioxaborolan-2-yl)pyridin-2-amine (76 mg, 0.172 mmol) to give the title compound (26.3 mg, 37.7% yield) as a pale yellow solid. ¹H NMR (500 MHz, DMSO-d₆) δ ppm 8.15 (d, J=8.19 Hz, 1H), 7.70 (s, 1H), 7.64 (d, J=10.15 Hz, 1H), 7.60 (d, J=8.19 Hz, 1H), 7.42 (d, J=8.07 Hz, 2H), 6.96 (d, J=8.68 Hz, 2H), 6.21 (s, 2H), 5.85 (s, 2H), 3.12-3.17 (m, 4H), 2.63-2.70 (m, 1H), 2.61 (s, 3H), 2.56-2.59 (m, 4H), 1.00 (d, J=6.48 Hz, 6H); LCMS: [M+H]⁺=488.45.

### Example 412: 6-(2-amino-6-fluoro-5-(4-(4-isopropylpiperazin-1-yl)phenyl)pyridin-3-yl)-4-chloroisoquinolin-1(2H)-one (I-466)

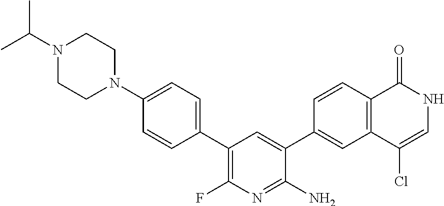 [SMILES: CC(C)N1CCN(c2ccc(-c3cc(-c4ccc5c(=O)[nH]cc(Cl)c5c4)c(N)nc3F)cc2)CC1]

Step 1: 6-bromo-4-chloroisoquinolin-1(2H)-one

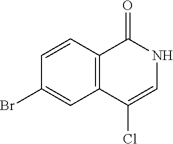 [SMILES: O=c1[nH]cc(Cl)c2cc(Br)ccc12]

To a solution of 6-bromo-2H-isoquinolin-1-one (600 mg, 2.68 mmol) in N,N-dimethylacetamide (14 ml) was added N-chlorosuccinimide (429 mg, 3.21 mmol). The mixture was stirred at 50° C. for 1 hour. The reaction was cooled to RT and diluted with water. The white suspension was filtered, washed with water and dried under vacuum to obtain 6-bromo-4-chloroisoquinolin-1(2H)-one (615 mg, 1.927 mmol, 72.0% yield, Purity (UV 254) 81%) as a white solid. The material was used in the next step without further purification. LCMS[M+H]⁺ 258.15.

Step 2: 4-chloro-6-(4,4,5,5-tetramethyl-1,3,2-dioxaborolan-2-yl)isoquinolin-1(2H)-one

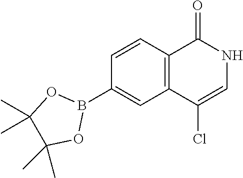 [SMILES: CC1(C)OB(c2ccc3c(=O)[nH]cc(Cl)c3c2)OC1(C)C]

Prepared from 6-bromo-4-chloroisoquinolin-1(2H)-one (132 mg, 0.511 mmol) to give a mixture of boronate and boronic acid which was used in the next step without further purification. LCMS[M+H]⁺ 306.24 Boronate, 224.17 Boronic acid.

Step 3: 6-(2-amino-6-fluoro-5-(4-(4-isopropylpiperazin-1-yl)phenyl)pyridin-3-yl)-4-chloroisoquinolin-1(2H)-one

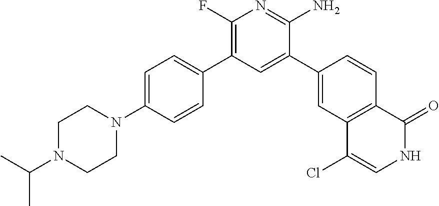 [SMILES: CC(C)N1CCN(c2ccc(-c3cc(-c4ccc5c(=O)[nH]cc(Cl)c5c4)c(N)nc3F)cc2)CC1]

Prepared from 4-chloro-6-(4,4,5,5-tetramethyl-1,3,2-dioxaborolan-2-yl)isoquinolin-1(2H)-one (74.6 mg, 0.110 mmol) and 3-bromo-6-fluoro-5-(4-(4-isopropylpiperazin-1-yl)phenyl)pyridin-2-amine (36 mg, 0.092 mmol) to give the title compound (10.5 mg, 23.3% yield) as a pale yellow solid. ¹H NMR (500 MHz, DMSO-d₆) b ppm 11.56 (br. s., 1H), 8.30 (d, J=8.31 Hz, 1H), 7.87 (s, 1H), 7.72 (d, J=8.19 Hz, 1H), 7.66 (d, J=10.03 Hz, 1H), 7.52 (d, J=5.14 Hz, 1H), 7.42 (d, J=7.70 Hz, 2H), 6.99 (d, J=6.36 Hz, 2H), 6.26 (br. s., 2H), 3.14 (br. s., 4H), 2.67-2.73 (m, 1H), 2.54-2.61 (m, 4H), 1.03 (br. s., 6H); LCMS: [M+H]⁺=492.21.

### Example 413: 7-(2-amino-5-(4-(4-(4,4-difluorobutyl)piperazin-1-yl)phenyl)-6-fluoropyridin-3-yl)-5-fluoro-2-methylquinazolin-4(3H)-one (I-467)

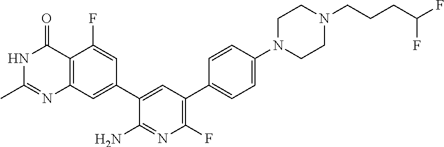 [SMILES: Cc1nc2cc(-c3cc(-c4ccc(N5CCN(CCCC(F)F)CC5)cc4)c(F)nc3N)cc(F)c2c(=O)[nH]1]

Step 1: 7-(2-amino-6-fluoro-5-(4-(piperazin-1-yl)phenyl)pyridin-3-yl)-5-fluoro-2-methylquinazolin-4(3H)-one

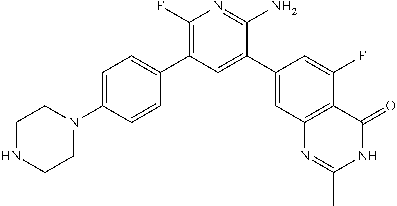 [SMILES: Cc1nc2cc(-c3cc(-c4ccc(N5CCNCC5)cc4)c(F)nc3N)cc(F)c2c(=O)[nH]1]

Prepared from 7-(2-amino-5-bromo-6-fluoropyridin-3-yl)-5-fluoro-2-methylquinazolin-4(3H)-one (60 mg, 0.163 mmol) and 4-piperazinylphenylboronic acid, pinacol ester (61.2 mg, 0.212 mmol) to give the title compound as a beige solid (35 mg, 48% yield). LCMS: [M+H]⁺=449.34.

Step 2: 7-(2-amino-5-(4-(4-(4,4-difluorobutyl)piperazin-1-yl)phenyl)-6-fluoropyridin-3-yl)-5-fluoro-2-methylquinazolin-4(3H)-one

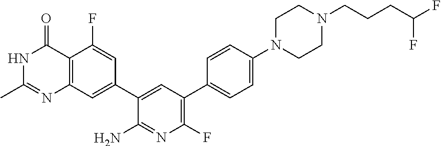 [SMILES: Cc1nc2cc(-c3cc(-c4ccc(N5CCN(CCCC(F)F)CC5)cc4)c(F)nc3N)cc(F)c2c(=O)[nH]1]

Prepared as per the procedure analogous to that of Example 86, step 3, by heating the intermediate from Example 91, step 1, 7-(2-amino-6-fluoro-5-(4-(piperazin-1-yl)phenyl)pyridin-3-yl)-5-fluoro-2-methylquinazolin-4(3H)-one (35 mg, 0.078 mmol) with 4-bromo-1,1-difluorobutane (27.0 mg, 0.156 mmol). The title compound was isolated as a pale-yellow solid (17 mg, 38%). ¹H NMR (500 MHz, METHANOL-d₄) δ ppm 7.69 (d, J=9.66 Hz, 1H), 7.56 (s, 1H), 7.46 (d, J=8.19 Hz, 2H), 7.35 (d, J=11.49 Hz, 1H), 7.05 (d, J=8.68 Hz, 2 H), 5.83-6.09 (m, 1H), 3.27 (d, J=5.01 Hz, 4H), 2.66-2.71 (m, 4H), 2.48-2.52 (m, 2H), 2.46 (s, 3H), 1.85-1.96 (m, 2H), 1.71-1.78 (m, 2H); LCMS: [M+H]⁺=541.41

### Example 414: 7-(2-amino-6-fluoro-5-(4-((1S,5R)-3-methyl-3-azabicyclo[3.1.0]hexan-1-yl)phenyl)pyridin-3-yl)-2-methylquinazolin-4(3H)-one (I-468)

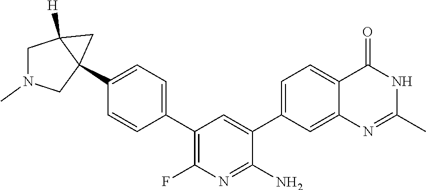

Prepared from 7-(2-amino-5-bromo-6-fluoropyridin-3-yl)-2-methylquinazolin-4(3H)-one (40 mg, 0.115 mmol) and (1S,5R)-3-methyl-1-(4-(4,4,5,5-tetramethyl-1,3,2-dioxaborolan-2-yl)phenyl)-3-azabicyclo[3.1.0]hexane (43.2 mg, 0.115 mmol) to give the title compound (43.6 mg, 86% yield). ¹H NMR (500 MHz, DMSO-d6) δ=12.22 (s, 1H), 8.13 (d, J=8.2 Hz, 1H), 7.73-7.64 (m, 2H), 7.58 (br d, J=8.6 Hz, 1H), 7.53-7.43 (m, 2H), 7.19 (br d, J=7.8 Hz, 2H), 6.33 (s, 2H), 3.35-3.27 (m, 1H), 3.10-2.96 (m, 1H), 0.68-2.56 (m, 1H), 2.41-2.28 (m, 7H), 1.88-1.77 (m, 1H), 1.36 (t, J=4.0 Hz, 1H), 0.89-0.71 (m, 1H). LCMS: [M+H]⁺=443.39.

### Example 415: 7-(2-amino-6-fluoro-5-(4-((1S,5R)-3-methyl-3-azabicyclo[3.1.0]hexan-1-yl)phenyl)pyridin-3-yl)-5-fluoro-2-methylquinazolin-4(3H)-one (I-469)

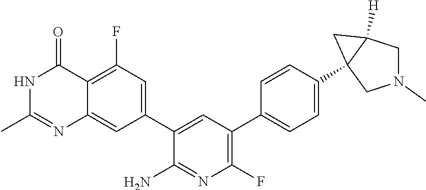

Prepared from 7-(2-amino-5-bromo-6-fluoropyridin-3-yl)-5-fluoro-2-methylquinazolin-4(3H)-one (40 mg, 0.109 mmol) and (1S,5R)-3-methyl-1-(4-(4,4,5,5-tetramethyl-1,3,2-dioxaborolan-2-yl)phenyl)-3-azabicyclo[3.1.0]hexane (41.1 mg, 0.109 mmol) to give the title compound (26.3 mg, 52.4% yield). ¹H NMR (500 MHz, DMSO-d₆) δ=12.25 (s, 1H), 7.69 (d, J=10.0 Hz, 1H), 7.53-7.44 (m, 3H), 7.33 (br d, J=11.2 Hz, 1H), 7.19 (br d, J=6.8 Hz, 2H), 6.42 (s, 2H), 3.32 (br dd, J=2.3, 4.3 Hz, 1H), 3.15-2.86 (m, 1H), 2.66-2.58 (m, 1H) 2.46-2.20 (m, 7H), 1.89-1.73 (m, 1H), 1.36 (t, J=4.0 Hz, 1H), 0.92-0.67 (m, 1H); [M+H]⁺=460.38;−111.13.

### Example 416: 6-(2-amino-6-fluoro-5-(4-((1S,5R)-3-methyl-3-azabicyclo[3.1.0]hexan-1-yl)phenyl)pyridin-3-yl)-4-fluoroisoquinolin-1(2H)-one (I-470)

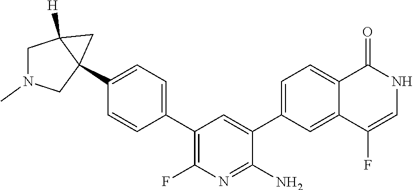 [SMILES: CN1C[C@@H]2C[C@]2(c2ccc(-c3cc(-c4ccc5c(=O)[nH]cc(F)c5c4)c(N)nc3F)cc2)C1]

Prepared from 6-(2-amino-5-bromo-6-fluoropyridin-3-yl)-4-fluoroisoquinolin-1(2H)-one (40 mg, 0.114 mmol) reacted with (1 S,5R)-3-methyl-1-(4-(4,4,5,5-tetramethyl-1,3,2-dioxaborolan-2-yl)phenyl)-3-azabicyclo[3.1.0]hexane (42.8 mg, 0.114 mmol), to give the title compound (14.7 mg, 28.9% yield). ¹H NMR (500 MHz, DMSO-d₆) δ=11.15 (br s, 1H), 8.27 (br d, J=8.2 Hz, 1H), 7.82 (s, 1H), 7.72 (br t, J=9.8 Hz, 2H), 7.48 (br d, J=7.8 Hz, 2H), 7.41 (br s, 1H), 7.18 (br d, J=8.1 Hz, 2H), 6.38 (s, 2H), 3.39-3.29 (m, 1H), 3.01 (br d, J=1.7 Hz, 1H), 2.64-2.2.4 (m, 2H), 2.32 (br s, 3H), 1.81 (br s, 1H), 1.40-1.33 (m, 1H), 0.78 (br d, J=2.1 Hz, 1H). LCMS: [M+H]⁺=445.08.

### Example 417: 6-(2-amino-6-fluoro-5-(4-((1S,5R)-3-methyl-3-azabicyclo[3.1.0]hexan-1-yl)phenyl)pyridin-3-yl)-4-methylisoquinolin-1(2H)-one (I-471)

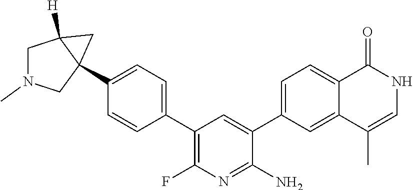 [SMILES: Cc1c[nH]c(=O)c2ccc(-c3cc(-c4ccc([C@]56C[C@H]5CN(C)C6)cc4)c(F)nc3N)cc12]

Prepared from 6-(2-amino-5-bromo-6-fluoropyridin-3-yl)-4-methylisoquinolin-1(2H)-one (40 mg, 0.115 mmol) and (1S,5R)-3-methyl-1-(4-(4,4,5,5-tetramethyl-1,3,2-dioxaborolan-2-yl)phenyl)-3-azabicyclo[3.1.0]hexane (43.3 mg, 0.115 mmol) to give the title compound (6.6 mg, 12.9% yield). ¹H NMR (500 MHz, DMSO-d₆) δ=11.09 (br d, J=5.4 Hz, 1H), 8.29 (d, J=8.2 Hz, 1H), 7.72 (s, 1H), 7.68 (d, J=9.9 Hz, 1H), 7.62 (br d, J=8.3 Hz, 1H), 7.46 (br d, J=7.7 Hz, 2H), 7.19 (br d, J=8.1 Hz, 2H), 7.04 (s, 1H), 3.39-3.29 (m, 1H), 3.13-3.00 (m, 1H), 2.71-2.49 (m, 2H), 2.42-2.30 (m, 3H), 2.23 (s, 3H), 1.90-1.74 (m, 1H), 1.33 (t, J=4.2 Hz, 1H), 0.90-0.72 (m, 1H). LCMS: [M+H]⁺=441.38,

### Example 418: 7-(2-amino-5-(4-(4-cyclobutylpiperazin-1-yl)phenyl)-6-fluoropyridin-3-yl)-2-methylquinazolin-4(3H)-one (I-472)

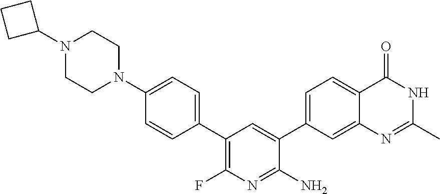 [SMILES: Cc1nc2cc(-c3cc(-c4ccc(N5CCN(C6CCC6)CC5)cc4)c(F)nc3N)ccc2c(=O)[nH]1]

Prepared from 7-(2-amino-5-bromo-6-fluoropyridin-3-yl)-2-methylquinazolin-4(3H)-one (50 mg, 0.115 mmol) and 1-cyclobutyl-4-(4-(4,4,5,5-tetramethyl-1,3,2-dioxaborolan-2-yl)phenyl)piperazine (39.2 mg, 0.115 mmol, in turn made from 4-piperazinylphenylboronic acid, pinacol ester and cyclobutanone in a manner similar to previous examples) to give the title compound (21.8 mg, 39.1% yield). ¹H NMR (500 MHz, DMSO-d₆) δ=12.20 (br s, 1H), 8.12 (d, J=8.2 Hz, 1H), 7.67 (s, 1H), 7.63 (d, J=10.1 Hz, 1H), 7.57 (br d, J=8.1 Hz, 1H), 7.45-7.37 (m, 2H), 6.96 (br d, J=8.8 Hz, 2H), 6.20 (s, 2H), 3.19-3.08 (m, 4H), 2.73 (quin, J=7.7 Hz, 1H), 2.42-2.32 (m, 7H), 2.05-1.93 (m, 2H), 1.86-1.75 (m, 2H), 1.71-1.58 (m, 2H). LCMS: [M+H]⁺=485.45.

### Example 419: 7-(2-amino-5-(4-(4-cyclobutylpiperazin-1-yl)phenyl)-6-fluoropyridin-3-yl)-5-fluoro-2-methylquinazolin-4(3H)-one (I-473)

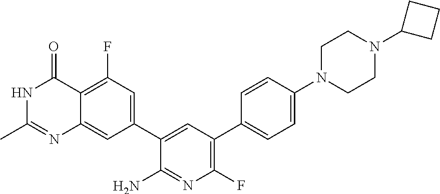 [SMILES: Cc1nc2cc(-c3cc(-c4ccc(N5CCN(C6CCC6)CC5)cc4)c(F)nc3N)cc(F)c2c(=O)[nH]1]

Prepared from 7-(2-amino-5-bromo-6-fluoropyridin-3-yl)-5-fluoro-2-methylquinazolin-4(3H)-one (40 mg, 0.109 mmol) and 1-cyclobutyl-4-(4-(4,4,5,5-tetramethyl-1,3,2-dioxaborolan-2-yl)phenyl)piperazine (37.3 mg, 0.109 mmol) to give the title compound (19.4 mg, 34.8% yield). ¹H-NMR (500 MHz, DMSO-d₆) δ=12.24 (br s, 1H), 7.66 (d, J=10.0 Hz, 1H), 7.50 (s, 1H), 7.43 (br d, J=8.3 Hz, 2H), 7.34 (br d, J=11.7 Hz, 1H), 6.97 (d, J=8.7 Hz, 2H), 6.31 (s, 2H), 3.23-3.09 (m, 4H), 2.74 (quin, J=7.6 Hz, 1H), 2.43-2.37 (m, 4H), 2.35 (s, 3H), 2.04-1.95 (m, 2H), 1.88-1.77 (m, 2H), 1.70-1.61 (m, 2H). LCMS: [M+H]⁺=503.37.

### Example 420: 7-(2-amino-5-(3-((dimethylamino)methyl)-4-morpholinophenyl)-6-fluoropyridin-3-yl)-2-methylquinazolin-4(3H)-one (I-474)

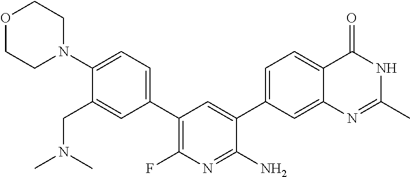 [SMILES: Cc1nc2cc(-c3cc(-c4ccc(N5CCOCC5)c(CN(C)C)c4)c(F)nc3N)ccc2c(=O)[nH]1]

Prepared from 7-(2-amino-5-bromo-6-fluoropyridin-3-yl)-2-methylquinazolin-4(3H)-one (50 mg, 0.115 mmol) and N,N-dimethyl-1-(2-morpholino-5-(4,4,5,5-tetramethyl-1,3,2-dioxaborolan-2-yl)phenyl)methanamine (39.7 mg, 0.115 mmol), to afford the title compound (21.5 mg, 37.8% yield). 1 H NMR (500 MHz, DMSO-d6) δ=12.21 (br s, 1H), 8.12 (d, J=8.2 Hz, 1H), 7.67 (s, 1H), 7.63 (d, J=10.1 Hz, 1H), 7.57 (br d, J=8.1 Hz, 1H), 7.53 (s, 1H), 7.43 (br d, J=8.4 Hz, 1H), 7.13 (d, J=8.3 Hz, 1H), 6.28 (s, 2H), 3.74 (br d, J=4.2 Hz, 4H), 3.45 (s, 2H), 2.94 (br s, 4H), 2.37 (s, 3H), 2.19 (s, 6H). LCMS: [M+H]⁺=489.46;

### Example 421: 7-(2-amino-5-(3-((dimethylamino)methyl)-4-morpholinophenyl)-6-fluoropyridin-3-yl)-5-fluoro-2-methylquinazolin-4(3H)-one (I-475)

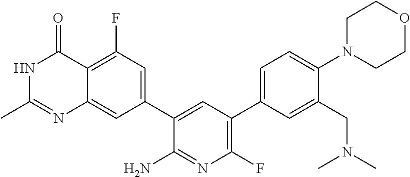 [SMILES: Cc1nc2cc(-c3cc(-c4ccc(N5CCOCC5)c(CN(C)C)c4)c(F)nc3N)cc(F)c2c(=O)[nH]1]

Prepared from 7-(2-amino-5-bromo-6-fluoropyridin-3-yl)-5-fluoro-2-methylquinazolin-4(3H)-one (40 mg, 0.109 mmol) and N,N-dimethyl-1-(2-morpholino-5-(4,4,5,5-tetramethyl-1,3,2-dioxaborolan-2-yl)phenyl)methanamine (37.7 mg, 0.109 mmol) to give the title compound (14.8 mg, 26.6% yield). ¹H NMR (500 MHz, DMSO-d₆) δ=12.24 (br s, 1H), 7.67 (br d, J=10.0 Hz, 1H), 7.54 (br s, 1H), 7.49 (s, 1H), 7.45 (br d, J=7.1 Hz, 1H), 7.33 (brd, J=11.5 Hz, 1H), 7.14 (br d, J=7.3 Hz, 1H), 6.39 (br s, 2H), 3.75 (br s, 4H), 3.58-3.41 (m, 2H), 2.93 (br s, 4H), 2.34 (s, 3H), 2.21 (s, 6H); LCMS: [M+H]⁺=507.44.

### Example 423: 6-(2-amino-5-(4-(4-(3,3-difluoropropyl)piperazin-1-yl)phenyl)-6-fluoropyridin-3-yl)-3,4-dihydroisoquinolin-1(2H)-one (I-476)

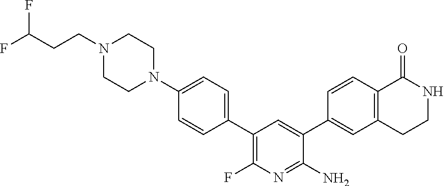 [SMILES: Nc1nc(F)c(-c2ccc(N3CCN(CCC(F)F)CC3)cc2)cc1-c1ccc2c(c1)CCNC2=O]

Prepared from 1-(4-bromophenyl)-4-(3,3-difluoropropyl)piperazine (50 mg, 0.157 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (78 mg, 0.204 mmol) to give the title compound as a white solid (58 mg, 71% yield). ¹H NMR (500 MHz, DMSO-d₆) δ ppm 7.85-7.98 (m, 2H), 7.59 (d, J=10.15 Hz, 1H), 7.36-7.50 (m, 4H), 6.98 (d, J=8.56 Hz, 2H), 5.96-6.28 (m, 3H), 3.41 (d, J=3.91 Hz, 2H), 3.17 (br. s., 4H), 2.96 (t, J=6.30 Hz, 2H), 2.54 (d, J=4.28 Hz, 4H), 2.47 (br. s., 2H), 1.98-2.12 (m, 2H); LCMS: [M+H]⁺=496.41.

### Example 424: 6-(2-amino-5-(4-(4-(3,3-difluoropropyl)piperazin-1-yl)phenyl)-6-fluoropyridin-3-yl)-7-fluoro-3,4-dihydroisoquinolin-1(2H)-one (I-477)

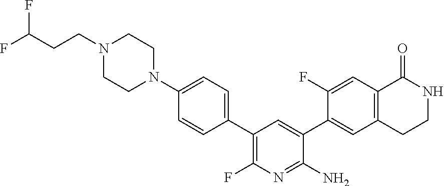 [SMILES: Nc1nc(F)c(-c2ccc(N3CCN(CCC(F)F)CC3)cc2)cc1-c1cc2c(cc1F)C(=O)NCC2]

Step 1: 1-(4-bromophenyl)-4-(3,3-dimethoxypropyl)piperazine

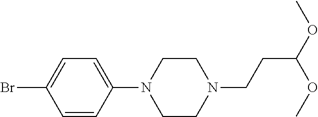 [SMILES: COC(CCN1CCN(c2ccc(Br)cc2)CC1)OC]

To a solution of 1-(4-bromophenyl)piperazine (15 g, 54.034 mmol) in ACN (150 mL), 3-bromo-1,1-dimethoxypropane (14.83 g, 81.05 mmol) and K₂CO₃ (26.09 g, 189.12 mmol) were added at room temperature and the reaction mass was stirred at RT for 16 h. The reaction mixture was poured into water (400 mL) and extracted with EtOAc (3×400 mL). The combined organic was dried over Na₂SO₄ and concentrated under vacuum to afford the crude which was purified by column chromatography in neutral alumina and the product was eluted in neat DCM to afford the product (12 g, 56.2% yield) as a white solid. LCMS: [M+2]⁺=345.2.

Step 2: 1-(4-bromophenyl)-4-(3,3-difluoropropyl)piperazine

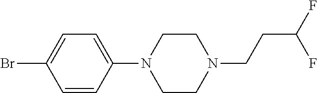 [SMILES: FC(F)CCN1CCN(c2ccc(Br)cc2)CC1]

To a solution of 1-(4-bromophenyl)-4-(3,3-dimethoxypropyl)piperazine(12 g, 34.15 mmol) in THF (120 mL), 10% dil. HCl (10 V) was added dropwise at room temperature. After completion of addition, reaction was stirred at 80° C. for 30 min. After completion of the reaction, the reaction mass was poured into sat. NaHCO₃ solution (400 mL) and extracted with DCM (2×100 mL). The combined organic layer was dried over anhydrous sodium sulfate filtered and cooled to −78° C., to this under nitrogen atmosphere, DAST (39.05 g, 242.26 mmol) was added dropwise. The reaction mixture was further stirred at same temperature for 3 h. After completion of reaction, reaction mass was poured into sat. NaHCO₃ solution (400 mL) and extracted with DCM (3×100 mL). The combine organic layer was washed with water (2×300 mL), brine (2×300 mL), dried over Na₂SO₄ and concentrated. The resulting crude product was purified by column chromatography in neutral alumina and the product was eluted in 14% EtOAc in hexanes to afford the title compound (1.7 g, 13.2% yield) as yellow solid. LCMS: [M+2]=321.05.

Step 3: 6-(2-amino-5-(4-(4-(3,3-difluoropropyl)piperazin-1-yl)phenyl)-6-fluoropyridin-3-1)-7-fluoro-3,4-dihydroisoquinolin-1(2H)-one

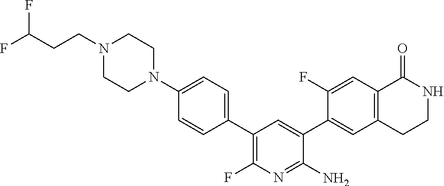 [SMILES: Nc1nc(F)c(-c2ccc(N3CCN(CCC(F)F)CC3)cc2)cc1-c1cc2c(cc1F)C(=O)NCC2]

Prepared from 1-(4-bromophenyl)-4-(3,3-difluoropropyl)piperazine (60 mg, 0.188 mmol) with 6-(2-amino-6-fluoro-5-(4,4,5,5-tetramethyl-1,3,2-dioxaborolan-2-yl)pyridin-3-yl)-3,4-dihydroisoquinolin-1(2H)-one (98 mg, 0.244 mmol) to give the title compound as a white solid (82 mg, 76% yield). ¹H NMR (500 MHz, DMSO-d₆) δ ppm 8.10 (br. s., 1H), 7.60 (dd, J=13.14, 10.21 Hz, 2H), 7.43 (d, J=6.85 Hz, 1H), 7.39 (d, J=8.31 Hz, 2H), 6.98 (d, J=8.56 Hz, 2H), 6.38-6.38 (m, 1H), 6.00-6.26 (m, 3H), 3.39-3.44 (m, 2H), 3.17 (d, J=4.40 Hz, 4H), 2.89-2.96 (m, 2H), 2.54 (d, J=4.89 Hz, 4H), 2.47 (br. s., 2H), 1.98-2.12 (m, 2H); LCMS: [M+H]⁺=514.21

### Example 425: 6-(2-amino-5-(4-(3,6-dihydro-2H-pyran-4-yl)-2-(5-oxopyrrolidin-3-yl)phenyl)-6-fluoropyridin-3-yl)-7-fluoro-3,4-dihydroisoquinolin-1(2H)-one (I-478)

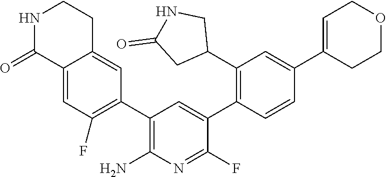 [SMILES: Nc1nc(F)c(-c2ccc(C3=CCOCC3)cc2C2CNC(=O)C2)cc1-c1cc2c(cc1F)C(=O)NCC2]

Step 1: 2-bromo-5-(3,6-dihydro-2H-pyran-4-yl)benzaldehyde

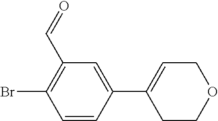 [SMILES: O=Cc1cc(C2=CCOCC2)ccc1Br]

2-Bromo-5-iodobenzaldehyde (7 g, 22.51 mmol), 3,6-dihydro-2H-pyran-4-boronic acid, pinacol ester (5.20 g, 24.77 mmol), tetrakis(triphenylphosphine)palladium(0) (1.301 g, 1.126 mmol) and K₂CO₃ (6.22 g, 6.43 mmol) were mixed together in 1,4-dioxane (70 mL) and water (14 mL) under vacuum. The reaction mixture was then stirred at 85° C. overnight. Additional tetrakis(triphenylphosphine)Palladium(0) (0.65 g, 0.563 mmol) was added and stirred at 85° C. for an additional 24 h. The mixture was filtered, rinsed with EtOAc. The filtrate was concentrated with silica gel and purified by column chromatography, eluting with 0-5% EtOAc in hexanes to give the product (4.13 g, 68.7% yield).

Step 2: Ethyl (E)-3-(2-bromo-5-(3,6-dihydro-2H-pyran-4-yl)phenyl)acrylate

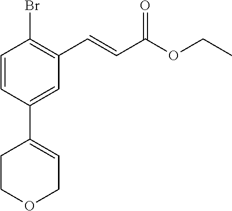 [SMILES: CCOC(=O)/C=C/c1cc(C2=CCOCC2)ccc1Br]

(Carbethoxymethylene)triphenyl phosphorane (7.00 g, 20.10 mmol) was mixed with 2-bromo-5-(3,6-dihydro-2H-pyran-4-yl)benzaldehyde (4.13 g, 15.46 mmol) in DCM (80 mL) under ice-water bath and stirred at r.t. overnight The reaction mixture was concentrated with silica gel, purified by column chromatography, eluted with 0-10% EtOac in hexanes to give ethyl (E)-3-(2-bromo-5-(3,6-dihydro-2H-pyran-4-yl)phenyl)acrylate 4.33 g. Yield=83%.

Step 3: Ethyl 3-(2-bromo-5-(3,6-dihydro-2H-pyran-4-yl)phenyl)-4-nitrobutanoate

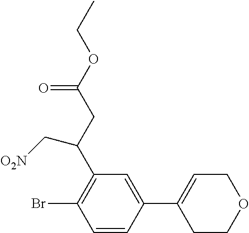 [SMILES: CCOC(=O)CC(C[N+](=O)[O-])c1cc(C2=CCOCC2)ccc1Br]

Ethyl (E)-3-(2-bromo-5-(3,6-dihydro-2H-pyran-4-yl)phenyl)acrylate (4.30 g, 12.75 mmol) was mixed with nitromethane (7.78 g, 128 mmol) in acetonitrile (50 ml) under ice water bath, then 1,8-diazabicyclo [5.4.0]undec-7-ene (1.941 g, 12.75 mmol) was added. After being stirred at r.t. overnight, the reaction mixture was concentrated and the residue was diluted with EtOAc, concentrated onto silica gel, purified by column chromatography, eluted with 0-30% EtOAc in hexanes to give the product (3.897 g, 77% yield) as colorless sticky oil.

Step 4: 4-(2-bromo-5-(3,6-dihydro-2H-pyran-4-yl)phenyl)pyrrolidin-2-one

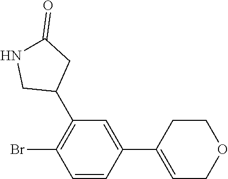 [SMILES: O=C1CC(c2cc(C3=CCOCC3)ccc2Br)CN1]

Ethyl 3-(2-bromo-5-(3,6-dihydro-2H-pyran-4-yl)phenyl)-4-nitrobutanoate (3.89 g, 9.77 mmol) was mixed with ammonium chloride (15.67 g, 293 mmol) in ethanol (75 ml) and water (35 ml) under 60° C. oil bath, then iron powder (5.45 g, 98 mmol) was added. The reaction mixture was stirred at 60° C. for 45 minutes. NaHCO₃ (24.62 g, 293 mmol) was added in portions and the reaction mixture was stirred under 70° C. oil bath overnight. The mixture was filtered, rinsed with 95% EtOH. The filtrate was concentrated by Rotavapor to remove EtOH, extracted with EtOAc twice. The organic layer was dried over MgSO₄, filtered, concentrated. The residue was triturated with ether to give the product (2.30 g, 73% yield) as off-white solid. LCMS: [M+H]⁺=322.22.

Step 5: 4-(5-(3,6-dihydro-2H-pyran-4-yl)-2-(4,4,5,5-tetramethyl-1,3,2-dioxaborolan-2-yl)phenyl)pyrrolidin-2-one

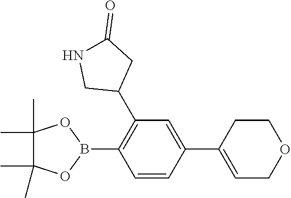 [SMILES: CC1(C)OB(c2ccc(C3=CCOCC3)cc2C2CNC(=O)C2)OC1(C)C]

4-(2-bromo-5-(3,6-dihydro-2H-pyran-4-yl)phenyl)pyrrolidin-2-one (210 mg, 0.652 mmol), bis(pinacolato)diboron (199 mg, 0.782 mmol), [1,1′-bis(diphenylphosphino)ferrocene] dichloropalladium(II) (47.7 mg, 0.065 mmol) and potassium acetate (128 mg, 1.304 mmol) were mixed together in 1,4-dioxane (12 mL) under vacuum and stirred under 85° C. oil bath overnight. Additional [1,1′-bis(diphenylphosphino)ferrocene] dichloropalladium(II) (30 mg, 0.041 mmol) and bis(pinacolato)diboron (50 mg, 0.197 mmol) were added, and stirred under 90 oC oil bath overnight again. The reaction mixture was filtered and the filtrate was concentrated purified by column chromatography, eluted with 0-100% EtOAc in hexanes to give the product (63 mg, 26.2% yield). LCMS: [M+H]⁺=370.49

Step 6: 6-(2-amino-5-(4-(3,6-dihydro-2H-pyran-4-yl)-2-(5-oxopyrrolidin-3-yl)phenyl)-6-fluoropyridin-3-yl)-7-fluoro-3,4-dihydroisoquinolin-1(2H)-one

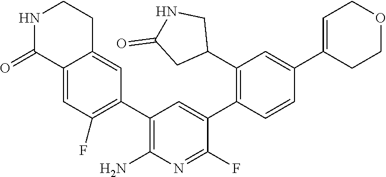 [SMILES: Nc1nc(F)c(-c2ccc(C3=CCOCC3)cc2C2CNC(=O)C2)cc1-c1cc2c(cc1F)C(=O)NCC2]

Prepared from 6-(2-amino-5-bromo-6-fluoropyridin-3-yl)-7-fluoro-3,4-dihydroisoquinolin-1(2H)-one (55.2 mg, 0.156 mmol) and 4-(5-(3,6-dihydro-2H-pyran-4-yl)-2-(4,4,5,5-tetramethyl-1,3,2-dioxaborolan-2-yl)phenyl)pyrrolidin-2-one (57.6 mg, 0.156 mmol) to give the title compound (030 mg, 36.6% yield)¹H NMR (500 MHz, METHANOL-d₄) 5=7.74 (d, J=10.0 Hz, 1H), 7.54 (s, 1H), 7.40 (br dd, J=7.3, 13.5 Hz, 3H), 7.22 (d, J=7.9 Hz, 1H), 6.25 (br s, 1H), 4.31 (br d, J=2.6 Hz, 2H), 3.94 (t, J=5.4 Hz, 2H), 3.77-3.67 (m, 2H), 3.53 (t, J=6.6 Hz, 2H), 3.01 (br t, J=6.5 Hz, 2H), 2.79-2.29 (m, 5H); LCMS: [M+H]⁺=517.41.

### Example 426: 6-(2-amino-6-fluoro-5-(2-(5-oxopyrrolidin-3-yl)-4-(tetrahydro-2H-pyran-4-yl)phenyl)pyridin-3-yl)-7-fluoro-3,4-dihydroisoquinolin-1(2H)-one (I-479)

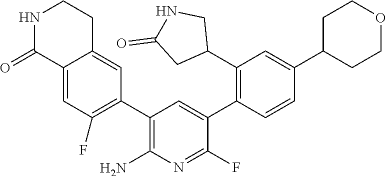 [SMILES: Nc1nc(F)c(-c2ccc(C3CCOCC3)cc2C2CNC(=O)C2)cc1-c1cc2c(cc1F)C(=O)NCC2]

6-(2-Amino-5-(4-(3,6-dihydro-2H-pyran-4-yl)-2-(5-oxopyrrolidin-3-yl)phenyl)-6-fluoropyridin-3-yl)-7-fluoro-3,4-dihydroisoquinolin-1(2H)-one (15 mg, 0.029 mmol) was mixed with 10% Palladium on carbon wet (3.09 mg, 0.029 mmol) in methanol(5 ml) and stirred under hydrogen balloon for 30 mins. The reaction mixture was filtered, and the filtrate was concentrated by Rotavapor. The residue was triturated with MeOH-ether to give the title compound (6.2 mg, 40.1% yield) as off-white solid. ¹H NMR (500 MHz, METHANOL-d₄) δ=7.73 (br d, J=10.0 Hz, 1H), 7.47-7.33 (m, 3H), 7.28-7.13 (m, 2H), 4.05 (br d, J=10.1 Hz, 2H), 3.78-3.65 (m, 2H), 3.64-3.50 (m, 5H), 3.01 (br t, J=6.3 Hz, 2H), 2.94-2.80 (m, 1H), 2.78-2.31 (m, 2H), 1.90-1.73 (m, 4H), LCMS: [M+H]⁺=519.48;

### Example 427: 6-(2-amino-5-(3-((dimethylamino)methyl)-4-morpholinophenyl)-6-fluoropyridin-3-yl)-4-fluoroisoquinolin-1(2H)-one (I-480)

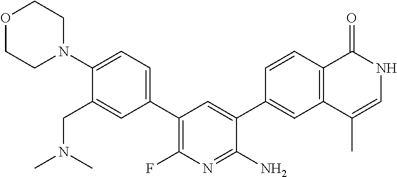 [SMILES: Cc1c[nH]c(=O)c2ccc(-c3cc(-c4ccc(N5CCOCC5)c(CN(C)C)c4)c(F)nc3N)cc12]

Prepared from 6-(2-amino-5-bromo-6-fluoropyridin-3-yl)-4-fluoroisoquinolin-1(2H)-one (45 mg, 0.128 mmol) and N,N-dimethyl-1-(2-morpholino-5-(4,4,5,5-tetramethyl-1,3,2-dioxaborolan-2-yl)phenyl)methanamine (53.1 mg, 0.153 mmol) to give the title compound (5.1 mg, 10.38 μmol, 8.1% yield) as a beige solid. ¹H NMR (500 MHz, DMSO-d₆) b ppm 11.16 (br. s., 1H), 8.27 (d, J=8.19 Hz, 1H), 7.82 (s, 1H), 7.73 (d, J=8.31 Hz, 1H), 7.68 (d, J=10.15 Hz, 1H), 7.53 (br. s., 1H), 7.40-7.46 (m, 2H), 7.14 (d, J=8.31 Hz, 1H), 6.36 (s, 2H), 3.74 (br. s., 4H), 3.47 (br. s., 2H), 2.93 (br. s., 4H), 2.20 (br. s., 6H); LCMS: [M+H]⁺=492.53.

### Example 428: 6-(2-amino-6-fluoro-5-(4-(4-isopropylpiperazin-1-yl)phenyl)pyridin-3-yl)-4,8-difluoro-3-methylisoquinolin-1(2H)-one (I-481)

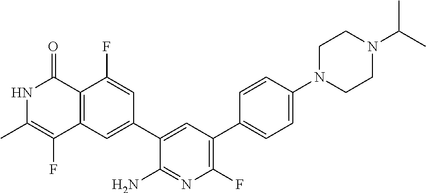 [SMILES: Cc1[nH]c(=O)c2c(F)cc(-c3cc(-c4ccc(N5CCN(C(C)C)CC5)cc4)c(F)nc3N)cc2c1F]

Step 1: 6-bromo-4,8-difluoro-3-methylisoquinolin-1(2H)-one

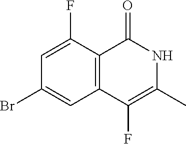

A vial was charged with 6-bromo-8-fluoro-3-methylisoquinolin-1(2H)-one (1.13 g, 4.41 mmol) and Selectfluor™ fluorinating reagent (1.641 g, 4.63 mmol). Methanol (10 mL) and acetonitrile (10 ml) were added, and the reaction was stirred at room temperature for 5 days. The white suspension was filtered, washed with water, and dried under vacuum to afford the product (1.01 g, 91% yield) as a white solid. LCMS: [M+H]⁺=274.14.

Step 2: (8-fluoro-3-methyl-1-oxo-1,2-dihydroisoquinolin-6-yl)boronic acid

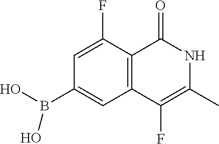 [SMILES: Cc1[nH]c(=O)c2c(F)cc(B(O)O)cc2c1F]

Prepared from 6-bromo-4,8-difluoro-3-methylisoquinolin-1(2H)-one (33 mg, 0.120 mmol) to give the boronic acid which was used in the next step without further purification. LCMS: [M+H]⁺=240.28.

Step 3: 6-(2-amino-6-fluoro-5-(4-(4-isopropylpiperazin-1-yl)phenyl)pyridin-3-yl)-4,8-difluoro-3-methylisoquinolin-1(2H)-one

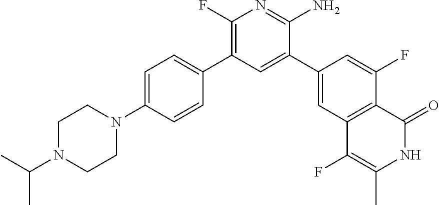 [SMILES: Cc1[nH]c(=O)c2c(F)cc(-c3cc(-c4ccc(N5CCN(C(C)C)CC5)cc4)c(F)nc3N)cc2c1F]

Prepared from (4,8-difluoro-3-methyl-1-oxo-1,2-dihydroisoquinolin-6-yl)boronic acid (25.3 mg, 0.106 mmol) and 3-bromo-6-fluoro-5-(4-(4-isopropylpiperazin-1-yl)phenyl)pyridin-2-amine (22 mg, 0.056 mmol) to give the title compound (4.8 mg, 16.9% yield) as a yellow solid. ¹H NMR (500 MHz, DMSO-d₆) δ ppm 11.24 (br. s., 1H), 7.68 (d, J=10.03 Hz, 1H), 7.53 (s, 1H), 7.37-7.45 (m, 3H), 6.97 (d, J=8.56 Hz, 2H), 6.35 (s, 2H), 3.15 (br. s., 4H), 2.67 (br. s., 1H), 2.58 (br. s., 4H), 2.22 (d, J=2.81 Hz, 3H), 1.01 (d, J=6.11 Hz, 6H); LCMS: [M+H]⁺=508.45.

### Example 429: 6-(2-amino-6-fluoro-5-(4-(4-isopropylpiperazin-1-yl)phenyl)pyridin-3-yl)-4,8-difluoroisoquinolin-1(2H)-one (I-482)

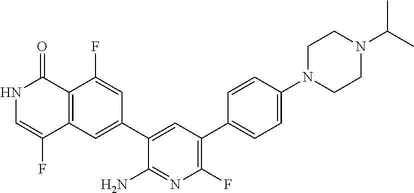 [SMILES: CC(C)N1CCN(c2ccc(-c3cc(-c4cc(F)c5c(=O)[nH]cc(F)c5c4)c(N)nc3F)cc2)CC1]

Step 1: 6-bromo-4,8-difluoroisoquinolin-1(2H)-one

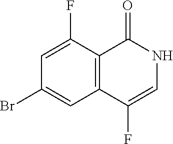 [SMILES: O=c1[nH]cc(F)c2cc(Br)cc(F)c12]

A vial was charged with 6-bromo-8-fluoro-1,2-dihydroisoquinolin-1-one (300 mg, 1.239 mmol) and Selectfluor™ fluorinating reagent (527 mg, 1.487 mmol). Methanol (3 mL) and acetonitrile (3 ml) were added, and the reaction was heated at 50° C. for 1 h. The reaction was concentrated in vacuo, dissolved in 1,2-dichloroethane (6 ml), and phosphorous (V) oxychloride (0.232 mL, 2.479 mmol) was added. The reaction was stirred at 50° C. for 1 h. The reaction was quenched into ice water and extracted with DCM (2x). The combined organic layers were dried with anhydrous sodium sulfate and concentrated to afford the product (306 mg, 95%) as a beige solid. The material was used in the next step without further purification. LCMS: [M+H]⁺=260.16.

Step 2: (4,8-difluoro-1-oxo-1,2-dihydroisoquinolin-6-yl)boronic acid

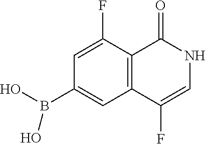 [SMILES: O=c1[nH]cc(F)c2cc(B(O)O)cc(F)c12]

Prepared from 6-bromo-4,8-difluoroisoquinolin-1(2H)-one (120 mg, 0.461 mmol) to give the boronic acid which was used in the next step without further purification. LCMS: [M+H]+=226.11.

Step 3: 6-(2-amino-6-fluoro-5-(4-(4-isopropylpiperazin-1-yl)phenyl)pyridin-3-yl)-4,8-difluoroisoquinolin-1(2H)-one

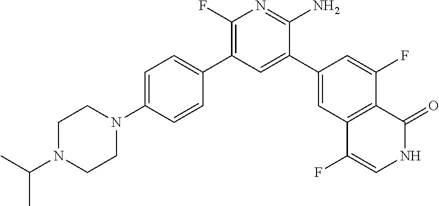 [SMILES: CC(C)N1CCN(c2ccc(-c3cc(-c4cc(F)c5c(=O)[nH]cc(F)c5c4)c(N)nc3F)cc2)CC1]

Prepared from (4,8-difluoro-1-oxo-1,2-dihydroisoquinolin-6-yl)boronic acid (33.5 mg, 0.149 mmol) and 3-bromo-6-fluoro-5-(4-(4-isopropylpiperazin-1-yl)phenyl)pyridin-2-amine (45 mg, 0.114 mmol) to give the title compound (20.2 mg, 35.8% yield) as a grey solid. ¹H NMR (500 MHz, DMSO-d6) δ ppm 11.14 (br. s., 1H), 7.71 (d, J=10.15 Hz, 1H), 7.61 (s, 1H), 7.49 (d, J=12.59 Hz, 1H), 7.46 (d, J=5.50 Hz, 1H), 7.42 (d, J=8.31 Hz, 2H), 6.97 (d, J=8.68 Hz, 2H), 6.39 (s, 2H), 3.13-3.16 (m, 4H), 2.65-2.70 (m, 1H), 2.56-2.59 (m, 4H), 1.00 (d, J=6.36 Hz, 6H); LCMS: [M+H]⁺=494.28.

### Example 430: 6-(2-amino-5-(4-(4-(cyclopropylmethyl)piperazin-1-yl)phenyl)-6-fluoropyridin-3-yl)-4,8-difluoroisoquinolin-1(2H)-one (I-483)

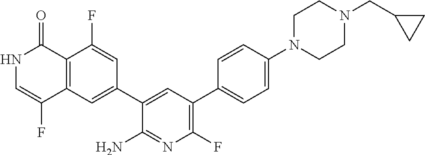 [SMILES: Nc1nc(F)c(-c2ccc(N3CCN(CC4CC4)CC3)cc2)cc1-c1cc(F)c2c(=O)[nH]cc(F)c2c1]

Prepared from (4,8-difluoro-1-oxo-1,2-dihydroisoquinolin-6-yl)boronic acid (34.0 mg, 0.151 mmol) and 3-chloro-5-(4-(4-(cyclopropylmethyl)piperazin-1-yl)phenyl)-6-fluoropyridin-2-amine (42 mg, 0.116 mmol) to give the title compound (17.9 mg, 30.4% yield) as a yellow solid. ¹H NMR (500 MHz, DMSO-d₆) δ ppm 11.14 (br. s., 1H), 7.71 (d, J=10.15 Hz, 1H), 7.61 (s, 1H), 7.49 (d, J=12.59 Hz, 1H), 7.46 (d, J=5.75 Hz, 1H), 7.42 (d, J=8.31 Hz, 2H), 6.98 (d, J=8.68 Hz, 2H), 6.39 (s, 2H), 3.14-3.20 (m, 4H), 2.55-2.60 (m, 4H), 2.22 (d, J=6.60 Hz, 2H), 0.81-0.90 (m, 1H), 0.46-0.50 (m, 2H), 0.10 (q, J=4.81 Hz, 2H); LCMS: [M+H]⁺=506.44.

### Example 431: 7-(2-amino-5-(4-(4-(4,4-difluorobutyl)piperazin-1-yl)phenyl)-6-fluoropyridin-3-yl)-2-methylquinazolin-4(3H)-one (I-484)

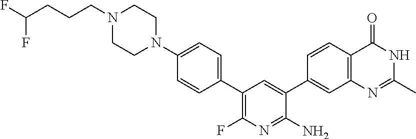 [SMILES: Cc1nc2cc(-c3cc(-c4ccc(N5CCN(CCCC(F)F)CC5)cc4)c(F)nc3N)ccc2c(=O)[nH]1]

Step 1: 7-(2-amino-6-fluoro-5-(4-(piperazin-1-yl)phenyl)pyridin-3-yl)-2-methylquinazolin-4(3H)-one

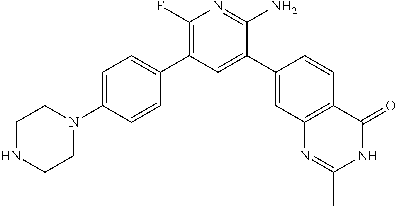 [SMILES: Cc1nc2cc(-c3cc(-c4ccc(N5CCNCC5)cc4)c(F)nc3N)ccc2c(=O)[nH]1]

Prepared from 7-(2-amino-6-fluoro-5-(4-(piperazin-1-yl)phenyl)pyridin-3-yl)-2-methylquinazolin-4(3H)-one (45 mg, 0.105 mmol) and 4-piperazinylphenylboronic acid, pinacol ester (64.4 mg, 0.223 mmol) to give the product as a yellow solid (45 mg, 61%); LCMS: [M+H]⁺=431.36

Step 2: 7-(2-amino-5-(4-(4-(4,4-difluorobutyl)piperazin-1-yl)phenyl)-6-fluoropyridin-3-yl)-2-methylquinazolin-4(3H)-one

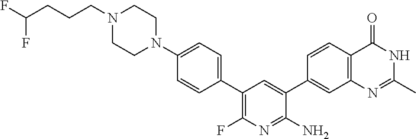 [SMILES: Cc1nc2cc(-c3cc(-c4ccc(N5CCN(CCCC(F)F)CC5)cc4)c(F)nc3N)ccc2c(=O)[nH]1]

Prepared from, 7-(2-amino-6-fluoro-5-(4-(piperazin-1-yl)phenyl)pyridin-3-yl)-2-methylquinazolin-4(3H)-one (45 mg, 0.105 mmol) and 4-bromo-1,1-difluorobutane (36.2 mg, 0.209 mmol) to give the title compound as a pale-yellow solid (30 mg, 52% yield). ¹H NMR (500 MHz, METHANOL-d₄) δ ppm 8.18 (d, J=8.19 Hz, 1H), 7.64 (s, 1H), 7.56 (dd, J=15.16, 9.05 Hz, 2H), 7.34 (d, J=8.31 Hz, 2H), 6.93 (d, J=8.68 Hz, 2H), 5.56-6.09 (m, 1 H), 3.16 (br. s., 4H), 2.57 (d, J=4.89 Hz, 4H), 2.35-2.42 (m, 5H), 1.72-1.84 (m, 2H), 1.62 (quin, J=7.55 Hz, 2H); LCMS: [M+H]⁺=523.46.

### Example 432: 7-(2-amino-5-(4-(4-(3,3-difluoropropyl)piperazin-1-yl)phenyl)-6-fluoropyridin-3-yl)-5-fluoro-2-methylquinazolin-4(3H)-one (I-485)

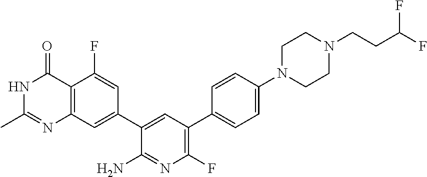 [SMILES: Cc1nc2cc(-c3cc(-c4ccc(N5CCN(CCC(F)F)CC5)cc4)c(F)nc3N)cc(F)c2c(=O)[nH]1]

Step 1: 1-(3,3-difluoropropyl)-4-(4-(4,4,5,5-tetramethyl-1,3,2-dioxaborolan-2-yl)phenyl)piperazine

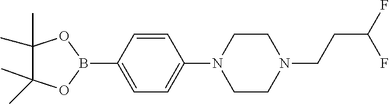 [SMILES: CC1(C)OB(c2ccc(N3CCN(CCC(F)F)CC3)cc2)OC1(C)C]

Prepared from 1-(4-bromophenyl)-4-(3,3-difluoropropyl)piperazine (225 mg, 0.705 mmol) and the resulting black solid was taken to the next step without any purification. (245 mg, 95% yield); LCMS: [M+H]⁺=367.42

Step 2: 7-(2-amino-5-(4-(4-(3,3-difluoropropyl)piperazin-1-yl)phenyl)-6-fluoropyridin-3-yl)-5-fluoro-2-methylquinazolin-4(3H)-one

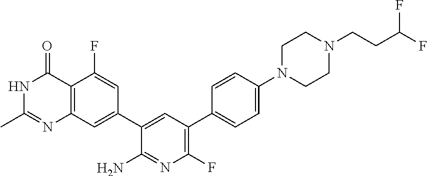 [SMILES: Cc1nc2cc(-c3cc(-c4ccc(N5CCN(CCC(F)F)CC5)cc4)c(F)nc3N)cc(F)c2c(=O)[nH]1]

Prepared from 1-(3,3-difluoropropyl)-4-(4-(4,4,5,5-tetramethyl-1,3,2-dioxaborolan-2-yl)phenyl)piperazine (59.9 mg, 0.163 mmol) and 7-(2-amino-5-bromo-6-fluoropyridin-3-yl)-5-fluoro-2-methylquinazolin-4(3H)-one (50 mg, 0.136 mmol) to yield the title compound as a pale-yellow solid (11.5 mg, 15% yield). ¹H NMR (500 MHz, METHANOL-d₄) δ ppm 7.58 (d, J=9.66 Hz, 1H), 7.45 (s, 1H), 7.34 (d, J=8.19 Hz, 2H), 7.23 (d, J=11.49 Hz, 1H), 6.82-7.01 (m, 2H), 5.73-6.04 (m, 1H), 3.11-3.17 (m, 4H), 2.54-2.63 (m, 4H), 2.50 (t, J=7.58 Hz, 2H), 2.34 (s, 3H), 1.95-2.06 (m, 2H); LCMS: [M+H]⁺=527.50.

### Example 433: 6-(2-amino-5-(3-((dimethylamino)methyl)-4-morpholinophenyl)-6-fluoropyridin-3-yl)-8-fluoro-3,4-dihydroisoquinolin-1(2H)-one (I-486)

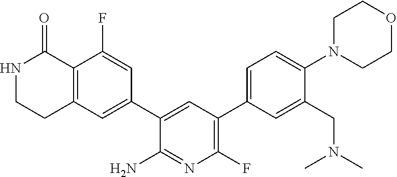 [SMILES: CN(C)Cc1cc(-c2cc(-c3cc(F)c4c(c3)CCNC4=O)c(N)nc2F)ccc1N1CCOCC1]

Step 1: N,N-dimethyl-1-(2-morpholino-5-(4,4,5,5-tetramethyl-1,3,2-dioxaborolan-2-yl)phenyl)methanamine

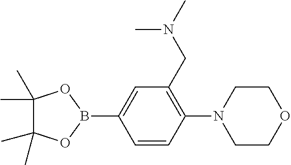 [SMILES: CN(C)Cc1cc(B2OC(C)(C)C(C)(C)O2)ccc1N1CCOCC1]

Prepared from 1-(5-bromo-2-morpholinophenyl)-N,N-dimethylmethanamine (120 mg, 0.401 mmol) to afford the product as a brown solid which was used without further purification (139 mg, 90% yield); LCMS: [M+H]⁺=347.37

Step 2: 6-(2-amino-5-(3-((dimethylamino)methyl)-4-morpholinophenyl)-6-fluoropyridin-3-yl)-8-fluoro-3,4-dihydroisoquinolin-1(2H)-one

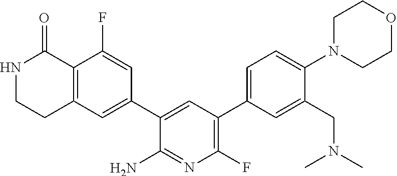 [SMILES: CN(C)Cc1cc(-c2cc(-c3cc(F)c4c(c3)CCNC4=O)c(N)nc2F)ccc1N1CCOCC1]

Prepared from N,N-dimethyl-1-(2-morpholino-5-(4,4,5,5-tetramethyl-1,3,2-dioxaborolan-2-yl)phenyl)methanamine (64 mg, 0.184 mmol) and 6-(2-amino-5-bromo-6-fluoropyridin-3-yl)-8-fluoro-3,4-dihydroisoquinolin-1(2H)-one (50 mg, 0.141 mmol) to afford the title compound as a beige solid (29 mg, 40% yield). ¹H NMR (500 MHz, DMSO-d₆) b ppm 8.00 (br. s., 1H), 7.63 (d, J=10.03 Hz, 1H), 7.52 (s, 1H), 7.43 (d, J=8.07 Hz, 1H), 7.30 (s, 1H), 7.26 (d, J=12.10 Hz, 1H), 7.14 (d, J=8.31 Hz, 1H), 6.38 (s, 2H), 3.75 (br. s., 4H), 3.45 (s, 2H), 3.25-3.32 (m, 2H), 2.94 (br. s., 6H), 2.19 (s, 6H); LCMS: [M+H]⁺=494.41.

### Example 434: 6-(2-amino-5-(3-((dimethylamino)methyl)-4-thiomorpholinophenyl)-6-fluoropyridin-3-yl)-3,4-dihydroisoquinolin-1(2H)-one (I-487)

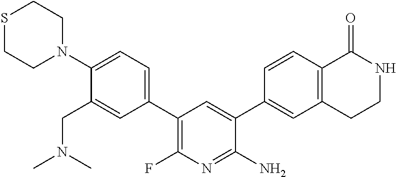 [SMILES: CN(C)Cc1cc(-c2cc(-c3ccc4c(c3)CCNC4=O)c(N)nc2F)ccc1N1CCSCC1]

Step 1: 5-bromo-2-thiomorpholinobenzaldehyde

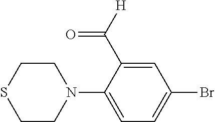 [SMILES: O=Cc1cc(Br)ccc1N1CCSCC1]

Prepared as per the procedure analogous to that of Example 33, step 1, by the reaction between 5-Bromo-2-fluorobenzaldehyde (5 g, 24.63 mmol) and Thiomorpholine (3.56 g, 34.5 mmol). The title compound was isolated as a pale-yellow solid (3.171 g, 45% yield). LCMS: [M+H]⁺=286.11.

Step 2: 1-(5-bromo-2-thiomorpholinophenyl)-N,N-dimethylmethanamine

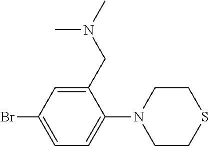 [SMILES: CN(C)Cc1cc(Br)ccc1N1CCSCC1]

Prepared from 5-bromo-2-thiomorpholinobenzaldehyde (2.305 g, 8.05 mmol) and dimethylamine, 2.0M solution in THF (16.11 ml, 32.2 mmol) as the amine. The title compound was isolated as a beige solid (2.51 g, 99% yield). LCMS: [M+H]⁺=315.27

Step 3: 6-(2-amino-5-(3-((dimethylamino)methyl)-4-thiomorpholinophenyl)-6-fluoropyridin-3-yl)-3,4-dihydroisoquinolin-1(2H)-one

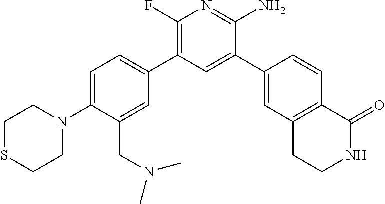 [SMILES: CN(C)Cc1cc(-c2cc(-c3ccc4c(c3)CCNC4=O)c(N)nc2F)ccc1N1CCSCC1]

Prepared from 1-(5-bromo-2-thiomorpholinophenyl)-N,N-dimethylmethanamine (50 mg, 0.159 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (79 mg, 0.206 mmol) to give the title compound as an off white solid (33 mg, 40% yield). ¹H NMR (500 MHz, DMSO-d₆) δ ppm 7.95 (br. s., 1H), 7.91 (d, J=7.82 Hz, 1H), 7.59 (d, J=10.03 Hz, 1H), 7.51 (s, 1H), 7.43-7.49 (m, 2H), 7.41 (d, J=8.07 Hz, 1H), 7.14 (d, J=8.31 Hz, 1H), 6.27 (s, 2 H), 3.39-3.47 (m, 4H), 3.16 (d, J=5.38 Hz, 4H), 2.95 (t, J=6.30 Hz, 2H), 2.77 (br. s., 4H), 2.19 (s, 6H); LCMS: [M+H]⁺=492.40

### Example 435: 6-(2-amino-5-(3-((dimethylamino)methyl)-4-thiomorpholinophenyl)-6-fluoropyridin-3-yl)-7-fluoro-3,4-dihydroisoquinolin-1(2H)-one (I-488)

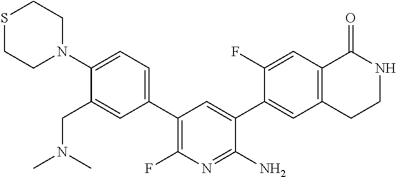 [SMILES: CN(C)Cc1cc(-c2cc(-c3cc4c(cc3F)C(=O)NCC4)c(N)nc2F)ccc1N1CCSCC1]

Prepared from 1-(5-bromo-2-thiomorpholinophenyl)-N,N-dimethylmethanamine (50 mg, 0.159 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (83 mg, 0.206 mmol) to give the title compound as a peach colored solid (39 mg, 46% yield). ¹H NMR (500 MHz, DMSO-d₆) δ ppm 8.11 (br. s., 1H), 7.60 (t, J=11.31 Hz, 2H), 7.49 (br. s., 1 H), 7.36-7.45 (m, 2H), 7.13 (d, J=8.19 Hz, 1H), 6.27 (br. s., 2H), 3.40-3.45 (m, 4H), 3.42 (br. s., 4H), 3.16 (br. s., 4H), 2.92 (t, J=6.05 Hz, 2H), 2.76 (br. s., 4H), 2.19 (s, 6H); LCMS: [M+H]⁺=510.39

### Example 436: 6-(2-amino-6-fluoro-5-(4-(4-isopropylpiperazin-1-yl)phenyl)pyridin-3-yl)-4-chloro-8-fluoroisoquinolin-1(2H)-one (I-489)

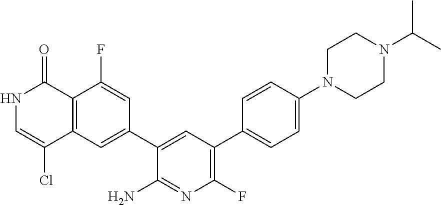 [SMILES: CC(C)N1CCN(c2ccc(-c3cc(-c4cc(F)c5c(=O)[nH]cc(Cl)c5c4)c(N)nc3F)cc2)CC1]

Step 1: 6-bromo-4-chloro-8-fluoroisoquinolin-1(2H)-one

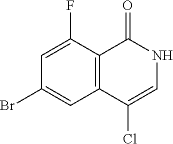 [SMILES: O=c1[nH]cc(Cl)c2cc(Br)cc(F)c12]

To a solution of 6-bromo-8-fluoro-1,2-dihydroisoquinolin-1-one (120 mg, 0.496 mmol) in N,N-dimethylacetamide (2 ml) was added N-chlorosuccinimide (79 mg, 0.595 mmol). The mixture was stirred at 50° C. for 1 hour then cooled to RT and diluted with water. The beige suspension was filtered, washed with water, and dried under vacuum to obtain the product (111 mg, 0.401 mmol, 81% yield) as a beige solid. The material was used at ˜80% purity (UV 254) in the next step without further purification. LCMS: [M+H]⁺=276.15.

Step 2: (4-chloro-8-fluoro-1-oxo-1,2-dihydroisoquinolin-6-yl)boronic acid

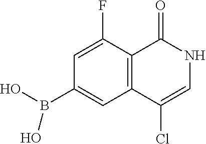 [SMILES: O=c1[nH]cc(Cl)c2cc(B(O)O)cc(F)c12]

Prepared from 6-bromo-4-chloro-8-fluoroisoquinolin-1(2H)-one (110 mg, 0.398 mmol) to give the boronic acid which was used in the next step without further purification. LCMS: [M+H]⁺=242.29.

Step 3: 6-(2-amino-6-fluoro-5-(4-(4-isopropylpiperazin-1-yl)phenyl)pyridin-3-yl)-4-chloro-8-fluoroisoquinolin-1(2H)-one

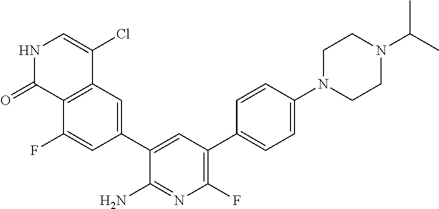 [SMILES: CC(C)N1CCN(c2ccc(-c3cc(-c4cc(F)c5c(=O)[nH]cc(Cl)c5c4)c(N)nc3F)cc2)CC1]

Prepared from (4-chloro-8-fluoro-1-oxo-1,2-dihydroisoquinolin-6-yl)boronic acid (31.9 mg, 0.132 mmol) and 3-bromo-6-fluoro-5-(4-(4-isopropylpiperazin-1-yl)phenyl)pyridin-2-amine (40 mg, 0.102 mmol) to give the title compound (5.3 mg, 10.2% yield) as a beige solid. ¹H NMR (500 MHz, DMSO-d₆) δ ppm 11.56 (br. s., 1H), 7.66-7.72 (m, 2H), 7.55 (d, J=2.81 Hz, 1H), 7.49 (d, J=12.35 Hz, 1H), 7.41 (d, J=8.07 Hz, 2H), 6.97 (d, J=8.80 Hz, 2H), 6.37 (s, 2H), 3.15 (br. s., 4H), 2.67 (br. s., 1H), 2.58 (br. s., 4H), 1.01 (d, J=6.36 Hz, 6H); LCMS: [M+H]⁺=510.45.

### Example 437: 7-(2-amino-5-(3-((dimethylamino)methyl)-4-(tetrahydro-2H-pyran-4-yl)phenyl)-6-fluoropyridin-3-yl)-5-fluoro-2-methylquinazolin-4(3H)-one (I-490)

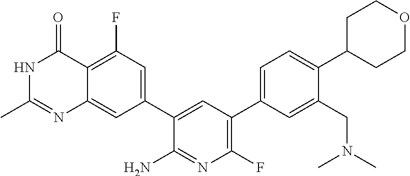 [SMILES: Cc1nc2cc(-c3cc(-c4ccc(C5CCOCC5)c(CN(C)C)c4)c(F)nc3N)cc(F)c2c(=O)[nH]1]

Prepared from 7-(2-amino-5-bromo-6-fluoropyridin-3-yl)-5-fluoro-2-methylquinazolin-4(3H)-one (40 mg, 0.109 mmol) and N,N-dimethyl-1-(2-(tetrahydro-2H-pyran-4-yl)-5-(4,4,5,5-tetramethyl-1,3,2-dioxaborolan-2-yl)phenyl)methanamine (37.6 mg, 0.109 mmol) to give the title compound (25.6 mg, 42.5% yield). ¹H NMR (500 MHz, METHANOL-d₄) δ=7.93 (d, J=9.7 Hz, 1H), 7.79 (s, 1H), 7.71 (br d, J=8.2 Hz, 1H), 7.67 (s, 1H), 7.62 (d, J=11.2 Hz, 1H), 7.56 (d, J=8.3 Hz, 1H), 4.52 (s, 2H), 4.06 (dd, J=3.7, 11.2 Hz, 2H), 3.68 (br t, J=11.2 Hz, 2H), 3.21-3.13 (m, 1H), 2.94 (s, 6H), 2.72 (s, 3H), 1.90 (dq, J=4.4, 12.5 Hz, 2H), 1.71 (br d, J=12.7 Hz, 2H); [M−HCl+H]⁺=506.50.

### Example 438: 6-(2-amino-6-fluoro-5-(4-morpholino-3-(pyrrolidin-1-ylmethyl)phenyl)pyridin-3-yl)-3,4-dihydroisoquinolin-1(2H)-one (I-491)

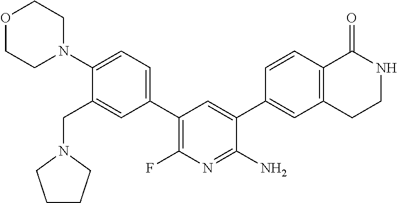 [SMILES: Nc1nc(F)c(-c2ccc(N3CCOCC3)c(CN3CCCC3)c2)cc1-c1ccc2c(c1)CCNC2=O]

Step 1: 4-(4-bromo-2-(pyrrolidin-1-ylmethyl)phenyl)morpholine

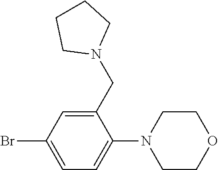 [SMILES: Brc1ccc(N2CCOCC2)c(CN2CCCC2)c1]

Prepared from 5-bromo-2-morpholinobenzaldehyde (400 mg, 1.481 mmol) and pyrrolidine 99% (0.486 ml, 5.92 mmol) to afford the product as an off white solid (508 mg, 95% yield). LCMS: [M+H]⁺=325.28.

Step 2: 6-(2-amino-6-fluoro-5-(4-morpholino-3-(pyrrolidin-1-ylmethyl)phenyl)pyridin-3-yl)-3,4-dihydroisoquinolin-1(2H)-one

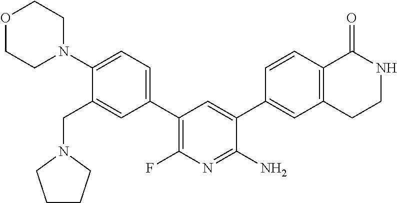 [SMILES: Nc1nc(F)c(-c2ccc(N3CCOCC3)c(CN3CCCC3)c2)cc1-c1ccc2c(c1)CCNC2=O]

Prepared from 4-(4-bromo-2-(pyrrolidin-1-ylmethyl)phenyl)morpholine (60 mg, 0.166 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (83 mg, 0.216 mmol) to give the title compound as a beige solid (67 mg, 76% yield). ¹H NMR (500 MHz, DMSO-d₆) δ ppm 7.94 (br. s., 1H), 7.88-7.93 (m, 1H), 7.59 (d, J=10.15 Hz, 1H), 7.52 (s, 1H), 7.47 (d, J=8.07 Hz, 1H), 7.45 (s, 1H), 7.42-7.42 (m, 1H), 7.42-7.42 (m, 1H), 7.40 (d, J=8.19 Hz, 1H), 7.11 (d, J=8.31 Hz, 1H), 6.25 (s, 2H), 3.73-3.82 (m, 4H), 3.65 (s, 2H), 3.40-3.45 (m, 2H), 2.91-3.01 (m, 6H), 2.47-2.50 (m, 4H), 1.68 (br. s., 4H); LCMS: [M+H]⁺=502.62

### Example 439: 6-(2-amino-6-fluoro-5-(4-morpholino-3-(pyrrolidin-1-ylmethyl)phenyl)pyridin-3-yl)-7-fluoro-3,4-dihydroisoquinolin-1(2H)-one (I-492)

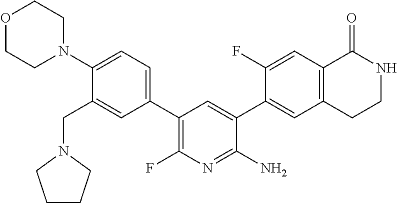 [SMILES: Nc1nc(F)c(-c2ccc(N3CCOCC3)c(CN3CCCC3)c2)cc1-c1cc2c(cc1F)C(=O)NCC2]

Prepared from 4-(4-bromo-2-(pyrrolidin-1-ylmethyl)phenyl)morpholine (60 mg, 0.166 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (87 mg, 0.216 mmol) to yield the title compound as a white solid (38 mg, 42% yield). ¹H NMR (500 MHz, DMSO-d₆) δ ppm 8.11 (br. s., 1H), 7.60 (t, J=11.00 Hz, 2H), 7.51 (s, 1H), 7.43 (d, J=7.09 Hz, 1H), 7.38 (d, J=8.19 Hz, 1H), 7.11 (d, J=8.31 Hz, 1H), 6.26 (s, 2H), 3.72-3.79 (m, 4H), 3.65 (s, 2H), 3.40-3.43 (m, 2H), 2.91-3.00 (m, 6H), 2.53-2.62 (m, 4H), 1.68 (br.s., 4H); LCMS: [M+H]⁺=520.54.

### Example 440: 6-(2-amino-5-(3-((dimethylamino)methyl)-4-(1,1-dioxidothiomorpholino)phenyl)-6-fluoropyridin-3-yl)-3,4-dihydroisoquinolin-1(2H)-one (1-493)

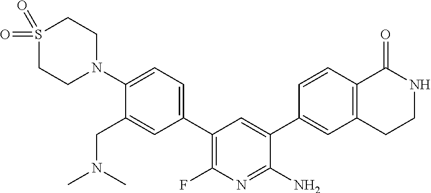 [SMILES: CN(C)Cc1cc(-c2cc(-c3ccc4c(c3)CCNC4=O)c(N)nc2F)ccc1N1CCS(=O)(=O)CC1]

Step 1: 4-(4-bromo-2-((dimethylamino)methyl)phenyl)thiomorpholine 1,1-dioxide

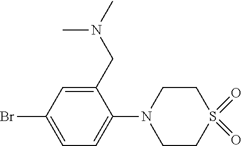 [SMILES: CN(C)Cc1cc(Br)ccc1N1CCS(=O)(=O)CC1]

3-Chloroperbenzoic acid (159 mg, 0.923 mmol) was added to a solution of 1-(5-bromo-2-thiomorpholinophenyl)-N,N-dimethylmethanamine (150 mg, 0.462 mmol) in CH₂Cl₂ (10 mL), cooled in an ice bath, and the reaction mixture was continuously stirred at the same temperature for 30 min. The reaction mixture was quenched with water (10 mL) and then extracted with CHCl₃/IPA (4:1) (3×10 mL). The combined organic layer was dried over sodium sulfate, filtered, concentrated and purified by silica gel chromatography, eluting with dichloromethane containing 0-10% MeOH and 0-1% NH₄OH to isolate the title product as a beige solid (93 mg, 58% yield). LCMS: [M+H]⁺=347.24.

Step 2: 6-(2-amino-5-(3-((dimethylamino)methyl)-4-(1,1-dioxidothiomorpholino)phenyl)-6-fluoropyridin-3-yl)-3,4-dihydroisoquinolin-1(2H)-one

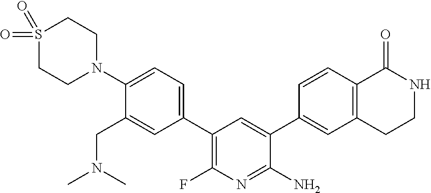 [SMILES: CN(C)Cc1cc(-c2cc(-c3ccc4c(c3)CCNC4=O)c(N)nc2F)ccc1N1CCS(=O)(=O)CC1]

Prepared from 4-(4-bromo-2-((dimethylamino)methyl)phenyl)thiomorpholine 1,1-dioxide (46 mg, 0.132 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (66 mg, 0.172 mmol) yield the title compound as a beige solid (46 mg, 63% yield). ¹H NMR (500 MHz, DMSO-d₆) δ ppm 8.04 (d, J=7.95 Hz, 1H), 7.91 (s, 1H), 7.73 (dd, J=14.86, 9.23 Hz, 2H), 7.51 (dd, J=8.07, 5.14 Hz, 2H), 7.46 (s, 1H), 4.60 (s, 2H), 3.62-3.70 (m, 2H), 3.56 (t, J=6.60 Hz, 2 H), 3.26 (t, J=11.19 Hz, 2H), 3.18 (s, 6H), 3.02-3.12 (m, 6H); LCMS: [M+H]⁺=524.49

### Example 441: 6-(2-amino-5-(4-(4-cyclopropylpiperazin-1-yl)-3-((dimethylamino)methyl)phenyl)-6-fluoropyridin-3-yl)-7-fluoro-3,4-dihydroisoquinolin-1(2H)-one (I-494)

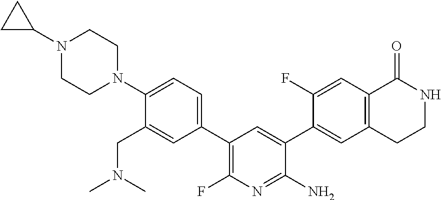 [SMILES: CN(C)Cc1cc(-c2cc(-c3cc4c(cc3F)C(=O)NCC4)c(N)nc2F)ccc1N1CCN(C2CC2)CC1]

Step 1: 5-bromo-2-(4-cyclopropylpiperazin-1-yl)benzaldehyde

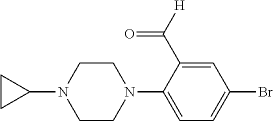 [SMILES: O=Cc1cc(Br)ccc1N1CCN(C2CC2)CC1]

Prepared as per the procedure analogous to that of Example 33, step 1, by the reaction between 5-Bromo-2-fluorobenzaldehyde (2.5 g, 12.31 mmol) and 1-cyclopropyl-piperazine (1.554 g, 12.31 mmol). The title compound was isolated as a beige solid (3.595 mg, 80% yield, 85% purity). LCMS: [M+H]⁺=309.25

Step 2: 1-(5-bromo-2-(4-cyclopropylpiperazin-1-yl)phenyl)-N,N-dimethylmethanamine

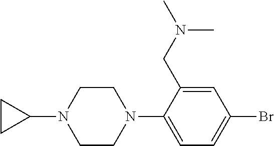 [SMILES: CN(C)Cc1cc(Br)ccc1N1CCN(C2CC2)CC1]

Prepared using 5-bromo-2-(4-cyclopropylpiperazin-1-yl)benzaldehyde (500 mg, 1.374 mmol) and dimethylamine, 2.0M solution in THF (2.75 ml, 5.50 mmol) to give the product as a beige solid (477 mg, 97% yield). LCMS: [M+H]⁺=338.34

Step 3: 6-(2-amino-5-(4-(4-cyclopropylpiperazin-1-yl)-3-((dimethylamino)methyl)phenyl)-6-fluoropyridin-3-yl)-7-fluoro-3,4-dihydroisoquinolin-1(2H)-one

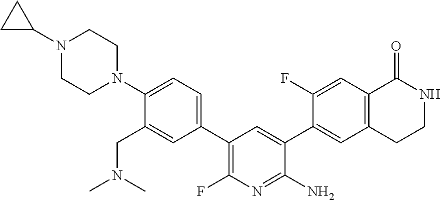 [SMILES: CN(C)Cc1cc(-c2cc(-c3cc4c(cc3F)C(=O)NCC4)c(N)nc2F)ccc1N1CCN(C2CC2)CC1]

Prepared from 1-(5-bromo-2-(4-cyclopropylpiperazin-1-yl)phenyl)-N,N-dimethylmethanamine (60 mg, 0.177 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (93 mg, 0.231 mmol) to yield the title compound as a beige solid (34.5 mg, 35% yield). ¹H NMR (500 MHz, METHANOL-d₄) δ ppm 7.75 (d, J=10.03 Hz, 1H), 7.69 (d, J=9.54 Hz, 1H), 7.58 (s, 1 H), 7.43 (dd, J=14.24, 7.52 Hz, 2H), 7.21 (d, J=8.31 Hz, 1H), 3.61-3.67 (m, 2H), 3.56 (t, J=6.66 Hz, 2H), 3.03 (t, J=6.42 Hz, 2H), 2.95 (br. s., 4H), 2.84 (br. s., 4H), 2.29 (s, 6H), 1.76-1.83 (m, 1H), 0.53-0.59 (m, 2H), 0.43-0.51 (m, 2H); LCMS: [M+H]⁺=533.58.

### Example 442: 6-(2-amino-5-(4-(4-cyclopropylpiperazin-1-yl)-3-((dimethylamino)methyl)phenyl)-6-fluoropyridin-3-yl)-3,4-dihydroisoquinolin-1(2H)-one (I-495)

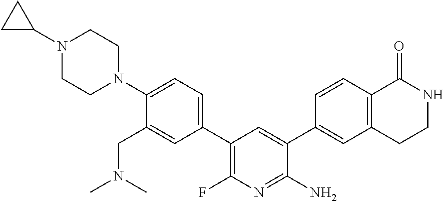 [SMILES: CN(C)Cc1cc(-c2cc(-c3ccc4c(c3)CCNC4=O)c(N)nc2F)ccc1N1CCN(C2CC2)CC1]

Prepared from 1-(5-bromo-2-(4-cyclopropylpiperazin-1-yl)phenyl)-N,N-dimethylmethanamine (50 mg, 0.148 mmol) from Example 99, step 2 with 6-(2-amino-6-fluoro-5-(4,4,5,5-tetramethyl-1,3,2-dioxaborolan-2-yl)pyridin-3-yl)-3,4-dihydroisoquinolin-1(2H)-one (73.6 mg, 0.192 mmol) to give the title compound as a beige solid (59 mg, 74% yield). ¹H NMR (500 MHz, METHANOL-d₄) δ ppm 8.01-8.08 (m, 1H), 7.66-7.73 (m, 1H), 7.58 (s, 1H), 7.50-7.54 (m, 1H), 7.42-7.48 (m, 2H), 7.17-7.24 (m, 1H), 3.61-3.66 (m, 2H), 3.56 (t, J=6.60 Hz, 2H), 3.06 (t, J=6.60 Hz, 2H), 2.93 (d, J=4.16 Hz, 4H), 2.83 (br. s., 4H), 2.25-2.33 (m, 6H), 1.78 (tt, J=6.71, 3.56 Hz, 1H), 0.52-0.58 (m, 2H), 0.44-0.51 (m, 2H); LCMS: [M+H]⁺=515.59

### Example 443: 6-(2-amino-6-fluoro-5-(4-morpholino-3-(morpholinomethyl)phenyl)pyridin-3-yl)-3,4-dihydroisoquinolin-1(2H)-one (I-496)

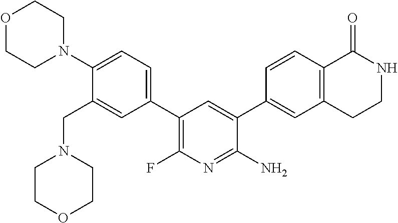 [SMILES: Nc1nc(F)c(-c2ccc(N3CCOCC3)c(CN3CCOCC3)c2)cc1-c1ccc2c(c1)CCNC2=O]

Prepared from 4-(4-bromo-2-(morpholinomethyl)phenyl)morpholine (50 mg, 0.147 mmol, in turn made from 5-bromo-2-morpholinobenzaldehyde and morpholine analogous to previous procedures) and 6-(2-amino-6-fluoro-5-(4,4,5,5-tetramethyl-1,3,2-dioxaborolan-2-yl)pyridin-3-yl)-3,4-dihydroisoquinolin-1(2H)-one (73.0 mg, 0.190 mmol) to afford the title compound as a white solid (52 mg, 65% yield). ¹H NMR (500 MHz, DMSO-d₆) δ ppm 7.87-8.00 (m, 2H), 7.60 (d, J=10.03 Hz, 1H), 7.53 (s, 1H), 7.38-7.49 (m, 3H), 7.16 (d, J=8.31 Hz, 1H), 6.27 (s, 2H), 3.75 (br. s., 4H), 3.54 (br. s., 6H), 3.40 (d, J=4.16 Hz, 2H), 2.96 (br. s., 6H), 2.42 (br. s., 4H); LCMS: [M+H]⁺=518.654

### Example 444: 6-(2-amino-6-fluoro-5-(4-morpholino-3-(morpholinomethyl)phenyl)pyridin-3-yl)-7-fluoro-3,4-dihydroisoquinolin-1(2H)-one (I-497)

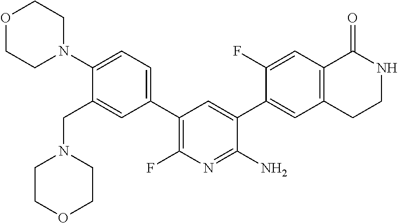 [SMILES: Nc1nc(F)c(-c2ccc(N3CCOCC3)c(CN3CCOCC3)c2)cc1-c1cc2c(cc1F)C(=O)NCC2]

Prepared from 4-(4-bromo-2-(pyrrolidin-1-ylmethyl)phenyl)morpholine (60 mg, 0.166 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (92 mg, 0.229 mmol) to give the title compound as a white solid (67 mg, 68% yield). ¹H NMR (500 MHz, DMSO-d₆) δ ppm 8.12 (br. s., 1H), 7.57-7.66 (m, 2H), 7.56-7.57 (m, 1H), 7.51 (s, 1H), 7.35-7.46 (m, 2H), 7.42 (dd, J=11.86, 7.83 Hz, 2H), 7.08-7.21 (m, 1H), 6.28 (s, 2H), 3.74 (d, J=4.16 Hz, 4H), 3.49-3.58 (m, 6H), 3.39-3.45 (m, 2H), 2.90-3.00 (m, 6H), 2.36-2.46 (m, 4H), 2.36-2.46 (m, 4H); LCMS: [M+H]⁺=536.46

### Example 445: 6-(2-amino-5-(3-((dimethylamino)methyl)-4-(1,1-dioxidothiomorpholino)phenyl)-6-fluoropyridin-3-yl)-7-fluoro-3,4-dihydroisoquinolin-1(2H)-one (I-498)

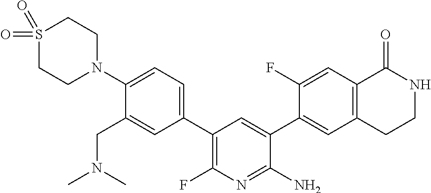 [SMILES: CN(C)Cc1cc(-c2cc(-c3cc4c(cc3F)C(=O)NCC4)c(N)nc2F)ccc1N1CCS(=O)(=O)CC1]

Prepared from 4-(4-bromo-2-((dimethylamino)methyl)phenyl)thiomorpholine 1,1-dioxide (46 mg, 0.132 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (69 mg, 0.172 mmol) to give the title compound as an off white solid (34 mg, 45% yield). ¹H NMR (500 MHz, DMSO-d₆) δ ppm 8.17 (br. s., 1H), 7.93 (br. s., 1H), 7.56-7.66 (m, 3H), 7.34-7.47 (m, 2H), 6.33 (br. s., 2H), 4.35 (br. s., 2H), 3.43-3.51 (m, 4H), 3.14 (br. s., 2H), 2.99 (br. s., 6H), 2.92 (d, J=8.80 Hz, 6H), 1.39 (br. s., 1H); LCMS: [M+H]⁺=542.35

### Example 446: 6-(2-amino-6-fluoro-5-(4-((3R,5S)-3,4,5-trimethylpiperazin-1-yl)phenyl)pyridin-3-yl)-4-fluoroisoquinolin-1(2H)-one (I-499)

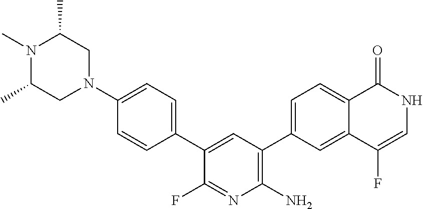 [SMILES: C[C@@H]1CN(c2ccc(-c3cc(-c4ccc5c(=O)[nH]cc(F)c5c4)c(N)nc3F)cc2)C[C@H](C)N1C]

Prepared from 6-(2-amino-5-bromo-6-fluoropyridin-3-yl)-4-fluoroisoquinolin-1(2H)-one (60 mg, and (2S,6R)-1,2,6-trimethyl-4-(4-(4,4,5,5-tetramethyl-1,3,2-dioxaborolan-2-yl)phenyl)piperazine (113 mg, 0.341 mmol to give the title compound as a grey solid (0.062 mmol, 36% yield). ¹H NMR (500 MHz, DMSO-d₆) δ 11.79-10.41 (m, 1H), 8.26 (dd, J=8.2, 1.7 Hz, 1H), 7.82 (d, J=0.9 Hz, 1H), 7.72 (dd, J=8.3, 1.3 Hz, 1H), 7.67 (d, J=10.1 Hz, 1H), 7.44-7.37 (m, 3H), 6.97 (d, J=8.8 Hz, 2H), 6.28 (s, 2H), 3.59 (br d, J=11.1 Hz, 2H), 2.40 (t, J=11.3 Hz, 2H), 2.28-2.20 (m, 2H), 2.18 (s, 3H), 1.07 (d, J=6.1 Hz, 6H); LCMS: [M+1]+=476.09.

### Example 447: 7-(2-amino-6-fluoro-5-(4-((3R,5S)-3,4,5-trimethylpiperazin-1-yl)phenyl)pyridin-3-yl)-2-methylquinazolin-4(3H)-one (I-500)

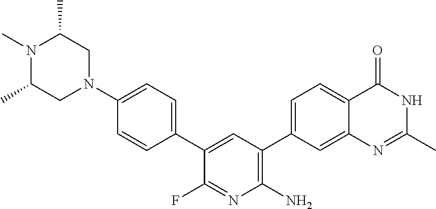

Prepared from 7-(2-amino-5-bromo-6-fluoropyridin-3-yl)-2-methylquinazolin-4(3H)-one (60 mg, 0.172 mmol) and (2S,6R)-1,2,6-trimethyl-4-(4-(4,4,5,5-tetramethyl-1,3,2-dioxaborolan-2-yl)phenyl)piperazine (114 mg, 0.344 mmol), ¹H NMR (500 MHz, DMSO-d₆) δ 12.64-11.87 (m, 1H), 8.12 (d, J=8.2 Hz, 1H), 7.66 (d, J=1.1 Hz, 1H), 7.62 (d, J=10.1 Hz, 1H), 7.57 (dd, J=8.1, 1.4 Hz, 1H), 7.40 (d, J=8.1 Hz, 2H), 6.97 (d, J=8.9 Hz, 2H), 6.21 (s, 2H), 3.59 (br d, J=11.1 Hz, 2H), 2.43-2.37 (m, 2H), 2.37 (s, 3H), 2.27-2.21 (m, 2H), 2.18 (s, 3H), 1.07 (d, J=6.1 Hz, 6H); LCMS: [M+1]⁺=473.46.

### Example 448: 7-(2-amino-6-fluoro-5-(4-((3R,5S)-3,4,5-trimethylpiperazin-1-yl)phenyl)pyridin-3-yl)-5-fluoro-2-methylquinazolin-4(3H)-one (I-501)

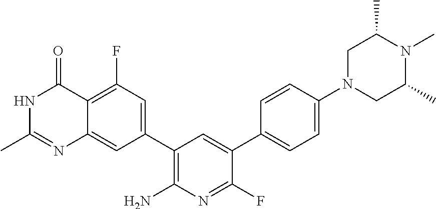

Prepared from 7-(2-amino-5-bromo-6-fluoropyridin-3-yl)-5-fluoro-2-methylquinazolin-4(3H)-one (60 mg, 0.163 mmol) and (2S,6R)-1,2,6-trimethyl-4-(4-(4,4,5,5-tetramethyl-1,3,2-dioxaborolan-2-yl)phenyl)piperazine (108 mg, 0.327 mmol), to give the title compound as a grey solid, (45 mg, 55.6% yield). ¹H NMR (500 MHz, DMSO-d₆) δ 12.59-11.82 (m, 1H), 7.65 (d, J=10.0 Hz, 1H), 7.48 (d, J=1.1 Hz, 1H), 7.41 (d, J=7.9 Hz, 2H), 7.33 (dd, J=11.7, 1.2 Hz, 1H), 6.97 (d, J=8.9 Hz, 2H), 6.31 (s, 2H), 3.59 (br d, J=11.0 Hz, 2H), 2.39 (t, J=11.3 Hz, 2H), 2.34 (s, 3H), 2.27-2.20 (m, 2H), 2.18 (s, 3H), 1.07 (d, J=6.1 Hz, 6H); LCMS: [M+1]⁺=491.44.

### Example 449: 6-(2-amino-5-(3-((dimethylamino)methyl)-4-morpholinophenyl)-6-fluoropyridin-3-yl)-4-methylisoquinolin-1(2H)-one (I-502)

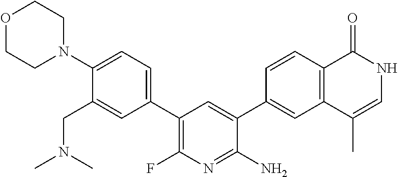 [SMILES: Cc1c[nH]c(=O)c2ccc(-c3cc(-c4ccc(N5CCOCC5)c(CN(C)C)c4)c(F)nc3N)cc12]

Step 1: N,N-dimethyl-1-(2-morpholino-5-(4,4,5,5-tetramethyl-1,3,2-dioxaborolan-2-yl)phenyl)methanamine

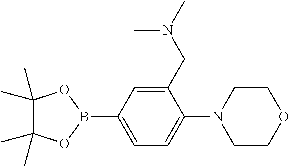 [SMILES: CN(C)Cc1cc(B2OC(C)(C)C(C)(C)O2)ccc1N1CCOCC1]

Prepared from 1-(5-bromo-2-morpholinophenyl)-N,N-dimethylmethanamine (132 mg, 0.441 mmol) to give a mixture of the boronate and boronic acid which was used in the next step without further purification. LCMS [M+H]+ 347.30 Boronate, 265.36 Boronic acid.

Step 2: 6-(2-amino-5-(3-((dimethylamino)methyl)-4-morpholinophenyl)-6-fluoropyridin-3-yl)-4-methylisoquinolin-1(2H)-one

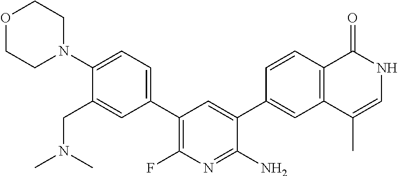 [SMILES: Cc1c[nH]c(=O)c2ccc(-c3cc(-c4ccc(N5CCOCC5)c(CN(C)C)c4)c(F)nc3N)cc12]

Prepared from 6-(2-amino-5-bromo-6-fluoropyridin-3-yl)-4-methylisoquinolin-1(2H)-one (30 mg, 0.086 mmol) and N,N-dimethyl-1-(2-morpholino-5-(4,4,5,5-tetramethyl-1,3,2-dioxaborolan-2-yl)phenyl)methanamine (35.8 mg, 0.103 mmol) to give the title compound (6.6 mg, 15.7% yield) as a white solid. ¹H NMR (500 MHz, DMSO-d₆) δ ppm 11.10 (br. s., 1H), 8.28 (d, J=8.19 Hz, 1H), 7.72 (s, 1H), 7.67 (d, J=10.15 Hz, 1H), 7.62 (d, J=8.19 Hz, 1H), 7.52 (s, 1H), 7.42 (d, J=8.44 Hz, 1H), 7.13 (d, J=8.31 Hz, 1H), 7.04 (s, 1H), 6.31 (s, 2H), 3.72-3.77 (m, 4H), 3.45 (s, 2H), 2.91-2.96 (m, 4H), 2.24 (s, 3H), 2.18 (s, 6H); LCMS: [M+H]⁺=488.45.

### Example 450: 6-(2-amino-5-(3-((dimethylamino)methyl)-4-(tetrahydro-2H-pyran-4-yl)phenyl)-6-fluoropyridin-3-yl)-4-fluoroisoquinolin-1(2H)-one (I-503)

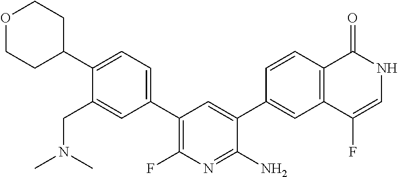 [SMILES: CN(C)Cc1cc(-c2cc(-c3ccc4c(=O)[nH]cc(F)c4c3)c(N)nc2F)ccc1C1CCOCC1]

Prepared from 6-(2-amino-5-bromo-6-fluoropyridin-3-yl)-4-fluoroisoquinolin-1(2H)-one (40 mg, 0.114 mmol) and N,N-dimethyl-1-(2-(tetrahydro-2H-pyran-4-yl)-5-(4,4,5,5-tetramethyl-1,3,2-dioxaborolan-2-yl)phenyl)methanamine (47.1 mg, 0.136 mmol) to give the title compound (23.6 mg, 42.4% yield) as a beige solid. ¹H NMR (500 MHz, DMSO-d₆) δ ppm 11.17 (br. s., 1H), 8.27 (dd, J=8.31, 1.83 Hz, 1H), 7.82 (d, J=1.22 Hz, 1H), 7.69-7.75 (m, 2H), 7.39-7.47 (m, 3H), 7.34 (d, J=8.07 Hz, 1H), 6.39 (s, 2H), 3.95 (dd, J=10.82, 3.48 Hz, 2H), 3.41-3.46 (m, 4H), 3.16-3.23 (m, 1H), 2.16 (s, 6H), 1.70 (qd, J=12.25, 4.10 Hz, 2H), 1.58-1.64 (m, 2H); LCMS: [M+H]⁺=491.46.

### Example 451: 6-(2-amino-5-(3-((dimethylamino)methyl)-4-(tetrahydro-2H-pyran-4-yl)phenyl)-6-fluoropyridin-3-yl)-8-fluoro-3-methylisoquinolin-1(2H)-one(I-504)

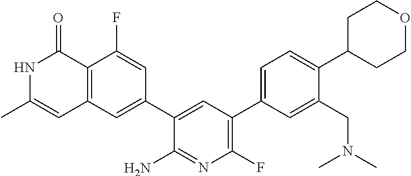 [SMILES: Cc1cc2cc(-c3cc(-c4ccc(C5CCOCC5)c(CN(C)C)c4)c(F)nc3N)cc(F)c2c(=O)[nH]1]

Step 1: 6-(2-amino-5-bromo-6-fluoropyridin-3-yl)-8-fluoro-3-methylisoquinolin-1(2H)-one

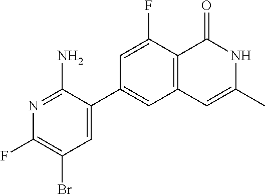 [SMILES: Cc1cc2cc(-c3cc(Br)c(F)nc3N)cc(F)c2c(=O)[nH]1]

Prepared from 5-bromo-6-fluoro-3-iodopyridin-2-amine (450 mg, 1.420 mmol) and (8-fluoro-3-methyl-1-oxo-1,2-dihydroisoquinolin-6-yl)boronic acid (408 mg, 1.846 mmol) to give the product (120 mg, 13.6% yield) as a white solid. The material contains de-brominated product, used as-is in the next step. LCMS: [M+H]⁺=366.23.

Step 2: 6-(2-amino-5-(3-((dimethylamino)methyl)-4-(tetrahydro-2H-pyran-4-yl)phenyl)-6-fluoropyridin-3-yl)-8-fluoro-3-methylisoquinolin-1(2H)-one AMC-4019-0489-08

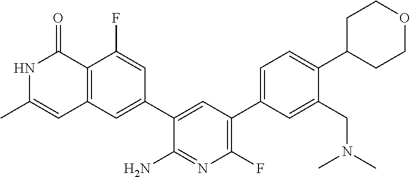 [SMILES: Cc1cc2cc(-c3cc(-c4ccc(C5CCOCC5)c(CN(C)C)c4)c(F)nc3N)cc(F)c2c(=O)[nH]1]

Prepared from 6-(2-amino-5-bromo-6-fluoropyridin-3-yl)-8-fluoro-3-methylisoquinolin-1(2H)-one (35 mg, 0.057 mmol) and N,N-dimethyl-1-(2-(tetrahydro-2H-pyran-4-yl)-5-(4,4,5,5-tetramethyl-1,3,2-dioxaborolan-2-yl)phenyl)methanamine (40.1 mg, 0.116 mmol) to give the title compound (22.7 mg, 46.5% yield) as a beige solid. 1H NMR (500 MHz, DMSO-d₆) δ ppm 11.25 (s, 1H), 7.68 (d, J=10.15 Hz, 1H), 7.42-7.46 (m, 2H), 7.39 (s, 1H), 7.34 (d, J=8.19 Hz, 1H), 7.22 (d, J=12.47 Hz, 1H), 6.39 (s, 2H), 6.34 (s, 1H), 3.95 (dd, J=10.70, 3.61 Hz, 2H), 3.41-3.47 (m, 4H), 3.16-3.23 (m, 1H), 2.19 (s, 3H), 2.16 (s, 6H), 1.66-1.76 (m, 2H), 1.58-1.65 (m, 2H); LCMS: [M+H]⁺=505.50.

### Example 452: 6-(2-amino-5-(3-((dimethylamino)methyl)-4-morpholinophenyl)-6-fluoropyridin-3-yl)-8-fluoro-3-methylisoquinolin-1(2H)-one (I-505)

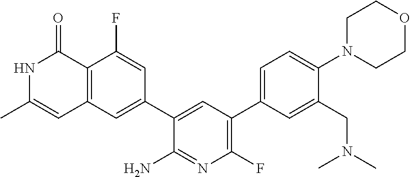 [SMILES: Cc1cc2cc(-c3cc(-c4ccc(N5CCOCC5)c(CN(C)C)c4)c(F)nc3N)cc(F)c2c(=O)[nH]1]

Prepared from 6-(2-amino-5-bromo-6-fluoropyridin-3-yl)-8-fluoro-3-methylisoquinolin-1(2H)-one (35 mg, 0.057 mmol) and N,N-dimethyl-1-(2-morpholino-5-(4,4,5,5-tetramethyl-1,3,2-dioxaborolan-2-yl)phenyl)methanamine (36.8 mg, 0.106 mmol) to give the title compound (19.1 mg, 39% yield) as a white solid. 1H NMR (500 MHz, DMSO-d₆) δ ppm 11.25 (s, 1H), 7.64 (d, J=10.03 Hz, 1H), 7.52 (s, 1H), 7.40-7.46 (m, 2H), 7.19-7.25 (m, 1H), 7.13 (d, J=8.44 Hz, 1H), 6.37 (s, 2H), 6.34 (s, 1H), 3.72-3.78 (m, 4H), 3.44 (s, 2H), 2.90-2.97 (m, 4H), 2.19 (s, 3H), 2.18 (s, 6H); LCMS: [M+H]⁺=506.44.

### Example 453: 6-(2-amino-5-(4-(cyclopropylmethoxy)-3-((dimethylamino)methyl)phenyl)-6-fluoropyridin-3-yl)-3,4-dihydroisoquinolin-1(2H)-one (1-506)

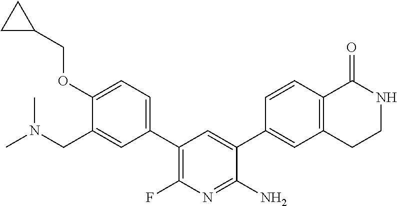 [SMILES: CN(C)Cc1cc(-c2cc(-c3ccc4c(c3)CCNC4=O)c(N)nc2F)ccc1OCC1CC1]

Prepared from 1-(5-bromo-2-(cyclopropylmethoxy)phenyl)-N,N-dimethylmethanamine (50 mg, 0.150 mmol, in turn made from 5-bromo-2-(cyclopropylmethoxy)benzaldehyde and dimethylamine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (74.5 mg, 0.194 mmol) afford the title compound as a beige solid (67 mg, 92% yield). ¹H NMR (500 MHz, METHANOL-d₄) δ ppm 8.02-8.09 (m, 1H), 7.67 (d, J=9.78 Hz, 1H), 7.52 (dd, J=8.01, 1.28 Hz, 1H), 7.42-7.50 (m, 3H), 7.01 (d, J=8.56 Hz, 1H), 3.91 (d, J=6.72 Hz, 2H), 3.60-3.67 (m, 2H), 3.56 (t, J=6.66 Hz, 2H), 3.07 (t, J=6.66 Hz, 2H), 2.27-2.35 (m, 6H), 1.30-1.35 (m, 1H), 0.62-0.67 (m, 2H), 0.38-0.42 (m, 2H); LCMS: [M+H]⁺=461.44

### Example 454: 6-(2-amino-5-(4-(cyclopropylmethoxy)-3-((dimethylamino)methyl)phenyl)-6-fluoropyridin-3-yl)-7-fluoro-3,4-dihydroisoquinolin-1(2H)-one (I-507)

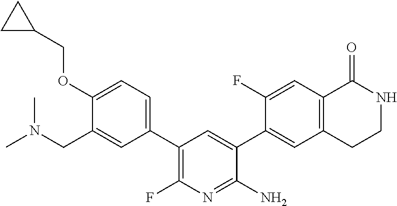 [SMILES: CN(C)Cc1cc(-c2cc(-c3cc4c(cc3F)C(=O)NCC4)c(N)nc2F)ccc1OCC1CC1]

Prepared from 1-(5-bromo-2-(cyclopropylmethoxy)phenyl)-N,N-dimethylmethanamine (50 mg, 0.150 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (78 mg, 0.194 mmol) to afford the title compound as a white solid (56 mg, 74% yield). ¹H NMR (500 MHz, METHANOL-d₄) δ ppm 7.75 (d, J=10.03 Hz, 1H), 7.64 (d, J=9.54 Hz, 1H), 7.39-7.48 (m, 3H), 6.97-7.02 (m, 1H), 3.90 (d, J=6.72 Hz, 2H), 3.62 (d, J=2.93 Hz, 2H), 3.55 (t, J=6.66 Hz, 2H), 3.03 (t, J=6.05 Hz, 2H), 2.24-2.41 (m, 6H), 1.30-1.35 (m, 1H), 0.59-0.69 (m, 2H), 0.35-0.45 (m, 2H); LCMS: [M+H]⁺=479.37

### Example 455: 6-(2-amino-5-(4-(cyclopropylmethoxy)-3-((4-methoxypiperidin-1-yl)methyl)phenyl)-6-fluoropyridin-3-yl)-3,4-dihydroisoquinolin-1(2H)-one (I-508)

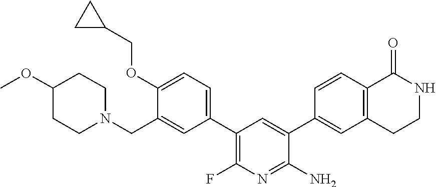 [SMILES: COC1CCN(Cc2cc(-c3cc(-c4ccc5c(c4)CCNC5=O)c(N)nc3F)ccc2OCC2CC2)CC1]

Prepared from 1-(5-bromo-2-(cyclopropylmethoxy)benzyl)-4-methoxypiperidine (45 mg, 0.127 mmol, in turn made from 5-bromo-2-(cyclopropylmethoxy)benzaldehyde and 4-methoxypiperidine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (63.3 mg, 0.165 mmol) to afford the title compound as a beige solid (36 mg, 51% yield). ¹H NMR (500 MHz, METHANOL-d₄) δ ppm 7.99-8.09 (m, 1H), 7.60-7.69 (m, 1H), 7.47-7.53 (m, 2H), 7.45 (s, 1H), 7.41 (d, J=8.31 Hz, 1H), 6.98 (dd, J=8.56, 1.71 Hz, 1H), 3.89 (d, J=6.72 Hz, 2H), 3.66 (s, 2H), 3.56 (t, J=6.60 Hz, 2H), 3.33 (s, 3H), 3.23-3.30 (m, 1H), 3.06 (t, J=6.42 Hz, 2H), 2.85 (br. s., 2H), 2.34 (t, J=9.17 Hz, 2H), 1.92 (d, J=10.64 Hz, 2H), 1.54-1.67 (m, 2H), 1.29-1.34 (m, 1H), 0.60-0.68 (m, 2H), 0.36-0.42 (m, 2H); LCMS: [M+H]⁺=531.51

### Example 456: 6-(2-amino-5-(4-(cyclopropylmethoxy)-3-((4-methoxypiperidin-1-yl)methyl)phenyl)-6-fluoropyridin-3-yl)-7-fluoro-3,4-dihydroisoquinolin-1(2H)-one (I-509)

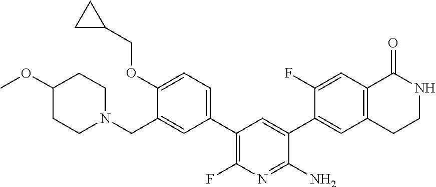 [SMILES: COC1CCN(Cc2cc(-c3cc(-c4cc5c(cc4F)C(=O)NCC5)c(N)nc3F)ccc2OCC2CC2)CC1]

Prepared from 1-(5-bromo-2-(cyclopropylmethoxy)benzyl)-4-methoxypiperidine (45 mg, 0.127 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (66.2 mg, 0.165 mmol) afford the title compound as a white solid (30 mg, 41% yield). ¹H NMR (500 MHz, METHANOL-d₄) δ ppm 7.75 (d, J=10.03 Hz, 1H), 7.64 (d, J=9.78 Hz, 1H), 7.48 (s, 1 H), 7.41 (d, J=7.09 Hz, 2H), 6.98 (d, J=8.56 Hz, 1H), 3.89 (d, J=6.72 Hz, 2H), 3.64-3.68 (m, 2H), 3.55 (t, J=6.66 Hz, 2H), 3.33 (s, 3H), 3.23-3.30 (m, 1H), 3.03 (t, J=6.48 Hz, 2H), 2.84 (br. s., 2H), 2.33 (t, J=9.60 Hz, 2H), 1.92 (d, J=10.88 Hz, 2H), 1.54-1.66 (m, 2H), 1.29-1.34 (m, 1H), 0.62-0.69 (m, 2H), 0.38 (q, J=4.93 Hz, 2H); LCMS: [M+H]⁺=549.56.

### Example 457: 6-(2-amino-6-fluoro-5-(3-((4-methoxypiperidin-1-yl)methyl)-4-morpholinophenyl)pyridin-3-yl)-3,4-dihydroisoquinolin-1(2H)-one (I-510)

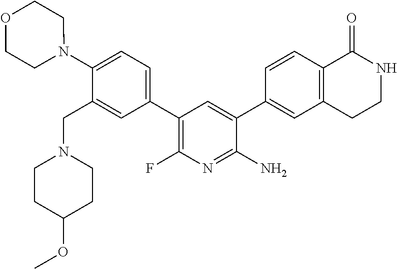 [SMILES: COC1CCN(Cc2cc(-c3cc(-c4ccc5c(c4)CCNC5=O)c(N)nc3F)ccc2N2CCOCC2)CC1]

Prepared from 4-(4-bromo-2-((4-methoxypiperidin-1-yl)methyl)phenyl)morpholine (60 mg, 0.162 mmol, in turn made from 5-bromo-2-morpholinobenzaldehyde and 4-methoxypiperidine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (81 mg, 0.211 mmol) give the title compound as an off white solid (58 mg, 62% yield). 1 H NMR (500 MHz, DMSO-d₆) δ ppm 7.95 (br. s., 1H), 7.91 (d, J=7.95 Hz, 1H), 7.60 (d, J=10.15 Hz, 1H), 7.52 (s, 1H), 7.47 (d, J=7.95 Hz, 1H), 7.45 (s, 1H), 7.41 (d, J=8.44 Hz, 1H), 7.14 (d, J=8.31 Hz, 1H), 6.26 (s, 2H), 3.73-3.79 (m, 4H), 3.51 (s, 2H), 3.40-3.44 (m, 2H), 3.21 (s, 3H), 3.17 (d, J=5.14 Hz, 1H), 2.93-2.99 (m, 6H), 2.67-2.75 (m, 2H), 2.14 (t, J=9.66 Hz, 2H), 1.80 (d, J=9.90 Hz, 2H), 1.32-1.43 (m, 2H); LCMS: [M+H]⁺=546.55.

### Example 458: 6-(2-amino-6-fluoro-5-(3-((4-methoxypiperidin-1-yl)methyl)-4-morpholinophenyl)pyridin-3-yl)-7-fluoro-3,4-dihydroisoquinolin-1(2H)-one (I-511)

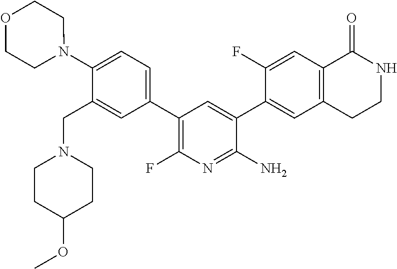 [SMILES: COC1CCN(Cc2cc(-c3cc(-c4cc5c(cc4F)C(=O)NCC5)c(N)nc3F)ccc2N2CCOCC2)CC1]

Prepared from 4-(4-bromo-2-((4-methoxypiperidin-1-yl)methyl)phenyl)morpholine (60 mg, 0.162 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (85 mg, 0.211 mmol) to yield the title compound as a white solid (42 mg, 44% yield). ¹H NMR (500 MHz, CHLOROFORM-d) δ ppm 7.90 (d, J=9.90 Hz, 1H), 7.60 (d, J=9.54 Hz, 1 H), 7.57 (s, 1H), 7.42 (d, J=8.19 Hz, 1H), 7.30 (d, J=6.60 Hz, 1H), 7.13 (d, J=8.31 Hz, 1H), 6.57 (br. s., 1H), 3.82-3.87 (m, 4H), 3.63 (td, J=6.57, 2.75 Hz, 2H), 3.57 (s, 2H), 3.32-3.36 (m, 3H), 3.20-3.26 (m, 1H), 3.03 (t, J=4.46 Hz, 6H), 2.76-2.85 (m, 2H), 2.20 (t, J=9.72 Hz, 2H), 1.84-1.92 (m, 2H), 1.50-1.58 (m, 2H); LCMS: [M+H]⁺=564.54.

### Example 459: 6-(2-amino-6-fluoro-5-(3-((4-methoxypiperidin-1-yl)methyl)-4-thiomorpholinophenyl)pyridin-3-yl)-3,4-dihydroisoquinolin-1(2H)-one (I-512)

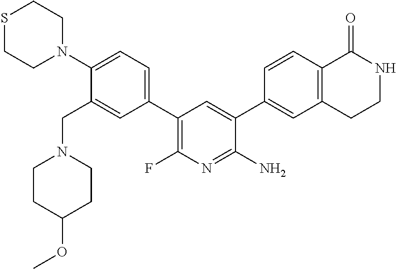 [SMILES: COC1CCN(Cc2cc(-c3cc(-c4ccc5c(c4)CCNC5=O)c(N)nc3F)ccc2N2CCSCC2)CC1]

Prepared from 4-(4-bromo-2-((4-methoxypiperidin-1-yl)methyl)phenyl)thiomorpholine (58 mg, 0.151 mmol, in turn made using 5-bromo-2-thiomorpholinobenzaldehyde and 4-methoxypiperidine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (75 mg, 0.196 mmol) to give the title compound as a white solid (68 mg, 76% yield). ¹H NMR (500 MHz, DMSO-d₆) δ ppm 7.95 (br. s., 1H), 7.91 (d, J=7.95 Hz, 1H), 7.60 (d, J=10.15 Hz, 1H), 7.50 (s, 1H), 7.46-7.49 (m, 1H), 7.45 (s, 1H), 7.41 (d, J=8.31 Hz, 1H), 7.14 (d, J=8.31 Hz, 1H), 6.27 (s, 2H), 3.47 (s, 2H), 3.40-3.43 (m, 2H), 3.20-3.22 (m, 3H), 3.17 (t, J=5.50 Hz, 5H), 2.95 (t, J=6.54 Hz, 2H), 2.73-2.78 (m, 4H), 2.67-2.73 (m, 2H), 2.14 (t, J=9.78 Hz, 2 H), 1.81 (d, J=10.03 Hz, 2H), 1.37 (d, J=9.41 Hz, 2H); LCMS: [M+H]⁺=562.53

### Example 460: 6-(2-amino-6-fluoro-5-(3-((4-methoxypiperidin-1-yl)methyl)-4-thiomorpholinophenyl)pyridin-3-yl)-7-fluoro-3,4-dihydroisoquinolin-1(2H)-one (I-513)

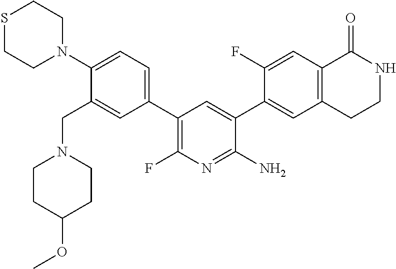 [SMILES: COC1CCN(Cc2cc(-c3cc(-c4cc5c(cc4F)C(=O)NCC5)c(N)nc3F)ccc2N2CCSCC2)CC1]

Prepared from 4-(4-bromo-2-((4-methoxypiperidin-1-yl)methyl)phenyl)thiomorpholine (58 mg, 0.151 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (79 mg, 0.196 mmol) to yield the title compound as a white solid (36 mg, 35% yield). 1H NMR (500 MHz, DMSO-d₆) δ ppm 8.11 (br. s., 1H), 7.55-7.65 (m, 2H), 7.48 (s, 1H), 7.43 (d, J=7.09 Hz, 1H), 7.40-7.40 (m, 1H), 7.39 (d, J=8.31 Hz, 1H), 7.08-7.20 (m, 1H), 6.14-6.38 (m, 2H), 3.47 (s, 2H), 3.40-3.43 (m, 2H), 3.21 (s, 3H), 3.17 (br. s., 5H), 2.92 (t, J=6.42 Hz, 2H), 2.73-2.79 (m, 4H), 2.68-2.73 (m, 2H), 2.13 (t, J=9.54 Hz, 2H), 1.80 (d, J=9.54 Hz, 2H), 1.37 (d, J=9.29 Hz, 2H); LCMS: [M+H]⁺=580.58.

### Example 461: 2-(6-amino-2-fluoro-5-(7-fluoro-1-oxo-1,2,3,4-tetrahydroisoquinolin-6-yl)pyridin-3-yl)-5-(3,6-dihydro-2H-pyran-4-yl)benzaldehyde (1-514)

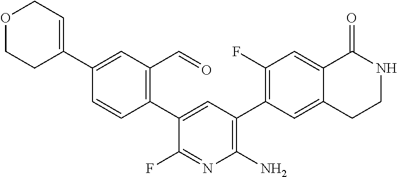 [SMILES: Nc1nc(F)c(-c2ccc(C3=CCOCC3)cc2C=O)cc1-c1cc2c(cc1F)C(=O)NCC2]

Prepared from 5-(3,6-dihydro-2H-pyran-4-yl)-2-(4,4,5,5-tetramethyl-1,3,2-dioxaborolan-2-yl)benzaldehyde (767 mg, 2.441 mmol) reacted with 6-(2-amino-5-bromo-6-fluoropyridin-3-yl)-7-fluoro-3,4-dihydroisoquinolin-1(2H)-one (908 mg, 2.56 mmol) to give the title compound (960 mg, 85% yield). ¹H NMR (500 MHz, DMSO-d₆) δ=9.98 (d, J=2.8 Hz, 1H), 8.12 (br s, 1H), 7.93 (d, J=2.0 Hz, 1H), 7.83 (dd, J=2.1, 8.2 Hz, 1H), 7.61 (dd, J=10.0, 13.3 Hz, 2H), 7.54-7.48 (m, 2H), 7.47-7.43 (m, 2H), 7.42-7.36 (m, 1H), 6.49 (s, 2H), 6.45 (br s, 1H), 4.27 (br d, J=2.7 Hz, 2H), 3.86 (t, J=5.4 Hz, 2H), 3.46-3.38 (m, 2H), 3.31 (br, s, 2H), 2.92 (br t, J=6.5 Hz, 2H); LCMS: [M+H]⁺=462.44.

### Example 462: 6-(2-amino-5-(3-((dimethylamino)methyl)-4-morpholinophenyl)-6-fluoropyridin-3-yl)-4-fluoro-3-methylisoquinolin-1(2H)-one (I-515)

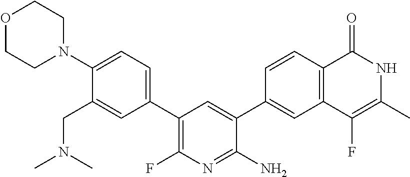 [SMILES: Cc1[nH]c(=O)c2ccc(-c3cc(-c4ccc(N5CCOCC5)c(CN(C)C)c4)c(F)nc3N)cc2c1F]

Step 1: 6-(2-amino-5-bromo-6-fluoropyridin-3-yl)-4-fluoro-3-methylisoquinolin-1(2H)-one

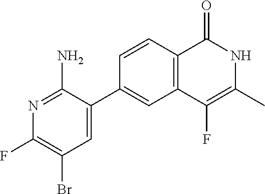 [SMILES: Cc1[nH]c(=O)c2ccc(-c3cc(Br)c(F)nc3N)cc2c1F]

Prepared from 5-bromo-6-fluoro-3-iodopyridin-2-amine (430 mg, 1.357 mmol) and 4-fluoro-3-methyl-6-(4,4,5,5-tetramethyl-1,3,2-dioxaborolan-2-yl)isoquinolin-1 (2H)-one (494 mg, 1.628 mmol) to give the product (443 mg, 89% yield) as a beige solid. LCMS: [M+H]⁺=366.23.

Step 2: 6-(2-amino-5-(3-((dimethylamino)methyl)-4-morpholinophenyl)-6-fluoropyridin-3-yl)-4-fluoro-3-methylisoquinolin-1(2H)-one

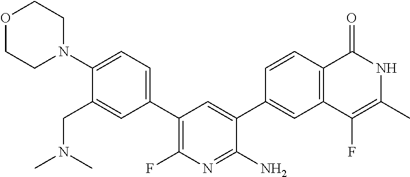 [SMILES: Cc1[nH]c(=O)c2ccc(-c3cc(-c4ccc(N5CCOCC5)c(CN(C)C)c4)c(F)nc3N)cc2c1F]

Prepared from 6-(2-amino-5-bromo-6-fluoropyridin-3-yl)-4-fluoro-3-methylisoquinolin-1(2H)-one (35 mg, 0.096 mmol) and N,N-dimethyl-1-(2-morpholino-5-(4,4,5,5-tetramethyl-1,3,2-dioxaborolan-2-yl)phenyl)methanamine (36.4 mg, 0.105 mmol) to give the title compound (9.4 mg, 19.5% yield) as a beige solid. ¹H NMR (500 MHz, DMSO-d₆) δ ppm 11.26 (br. s., 1H), 8.22 (dd, J=8.19, 1.59 Hz, 1H), 7.74 (s, 1H), 7.58-7.69 (m, 4 H), 7.54 (br. s., 2H), 7.44 (d, J=6.24 Hz, 1H), 7.14 (d, J=8.07 Hz, 1H), 6.35 (br. s., 2H), 3.75 (d, J=3.67 Hz, 4H), 3.47 (br. s., 2H), 2.93 (br. s., 4H), 2.24 (d, J=2.81 Hz, 3H), 2.20 (br. s., 3H); LCMS: [M+H]⁺=506.44.

### Example 463: 6-(2-amino-6-fluoro-5-(4-methoxy-3-((3-methoxyazetidin-1-yl)methyl)phenyl)pyridin-3-yl)-3,4-dihydroisoquinolin-1(2H)-one (I-516)

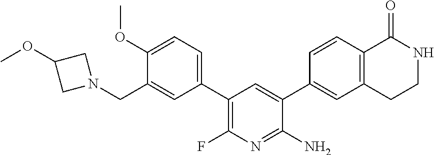 [SMILES: COc1ccc(-c2cc(-c3ccc4c(c3)CCNC4=O)c(N)nc2F)cc1CN1CC(OC)C1]

Prepared from 1-(5-bromo-2-methoxybenzyl)-3-methoxyazetidine (60 mg, 0.185 mmol, in turn made using 5-bromo-2-methoxybenzaldehyde and 3-methoxyazetidine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (92 mg, 0.240 mmol) to afford the title compound as an off white solid (53.5 mg, 60% yield). ¹H NMR ((500 MHz, METHANOL-d₄) ppm 8.06 (d, J=7.95 Hz, 1H), 7.66 (d, J=9.78 Hz, 1H), 7.53 (dd, J=7.95, 1.59 Hz, 1H), 7.44-7.49 (m, 2H), 7.44-7.49 (m, 2H), 7.42 (s, 1H), 7.02-7.09 (m, 1H), 4.01-4.09 (m, 1H), 3.87-3.92 (m, 3H), 3.74 (s, 2H), 3.61-3.67 (m, 2H), 3.54-3.60 (m, 2H), 3.22-3.29 (m, 3H), 3.22-3.29 (m, 3H), 3.10-3.15 (m, 2H), 3.08 (t, J=6.66 Hz, 2H); LCMS: [M+H]⁺=463.51.

### Example 464: 6-(2-amino-6-fluoro-5-(4-methoxy-3-((3-methoxyazetidin-1-yl)methyl)phenyl)pyridin-3-yl)-7-fluoro-3,4-dihydroisoquinolin-1(2H)-one (I-517)

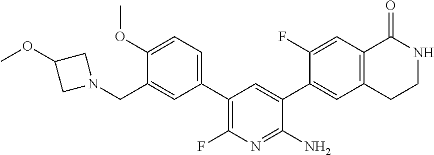 [SMILES: COc1ccc(-c2cc(-c3cc4c(cc3F)C(=O)NCC4)c(N)nc2F)cc1CN1CC(OC)C1]

Prepared from 1-(5-bromo-2-methoxybenzyl)-3-methoxyazetidine (60 mg, 0.185 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (96 mg, 0.240 mmol) to afford the title compound as a white solid (36.5 mg, 39% yield). ¹H NMR ((500 MHz, METHANOL-d₄) b ppm 7.71-7.79 (m, 1H), 7.60-7.66 (m, 1H), 7.36-7.45 (m, 3H), 6.98-7.07 (m, 1H), 3.99-4.07 (m, 1H), 3.84-3.90 (m, 3H), 3.72 (s, 2H), 3.59-3.64 (m, 2H), 3.52-3.58 (m, 2H), 3.21-3.27 (m, 3H), 3.07-3.12 (m, 2H), 2.99-3.06 (m, 2H); LCMS: [M+H]⁺=481.50.

### Example 465: 6-(2-amino-6-fluoro-5-(4-methoxy-3-((4-methoxypiperidin-1-yl)methyl)phenyl)pyridin-3-yl)-3,4-dihydroisoquinolin-1(2H)-one (I-518)

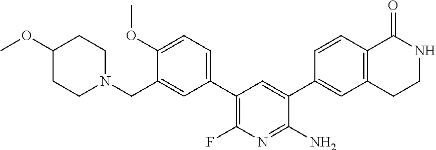 [SMILES: COc1ccc(-c2cc(-c3ccc4c(c3)CCNC4=O)c(N)nc2F)cc1CN1CCC(OC)CC1]

Prepared from 1-(5-bromo-2-methoxybenzyl)-4-methoxypiperidine (60 mg, 0.179 mmol, in turn made from 5-bromo-2-methoxybenzaldehyde and 4-methoxypiperidine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (89 mg, 0.233 mmol) to afford the title compound as an off white solid (66 mg, 71% yield). ¹H NMR ((500 MHz, METHANOL-d₄) δ ppm 7.96-8.08 (m, 1H), 7.59-7.66 (m, 1H), 7.47-7.51 (m, 1H), 7.46 (s, 1H), 7.40-7.45 (m, 2H), 7.01 (d, J=8.56 Hz, 1H), 3.82-3.89 (m, 3H), 3.58 (s, 2H), 3.54 (t, J=6.66 Hz, 2H), 3.31 (s, 3H), 3.25 (dt, J=8.16, 4.29 Hz, 1H), 3.04 (t, J=6.66 Hz, 2H), 2.71-2.88 (m, 2H), 2.19-2.36 (m, 2H), 1.84-1.94 (m, 2H), 1.52-1.62 (m, 2H); LCMS: [M+H]⁺=491.52.

### Example 466: 6-(2-amino-6-fluoro-5-(4-methoxy-3-((4-methoxypiperidin-1-yl)methyl)phenyl)pyridin-3-yl)-7-fluoro-3,4-dihydroisoquinolin-1(2H)-one (I-519)

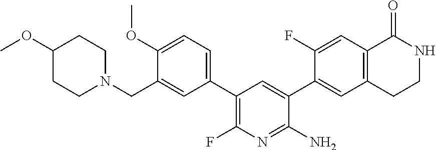 [SMILES: COc1ccc(-c2cc(-c3cc4c(cc3F)C(=O)NCC4)c(N)nc2F)cc1CN1CCC(OC)CC1]

Prepared from 1-(5-bromo-2-methoxybenzyl)-4-methoxypiperidine (60 mg, 0.179 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (94 mg, 0.233 mmol) to afford the title compound as a white solid (36 mg, 37.5% yield). ¹H NMR ((500 MHz, METHANOL-d₄) b ppm 7.76 (d, J=10.03 Hz, 1H), 7.66 (d, J=9.78 Hz, 1H), 7.45-7.51 (m, 2H), 7.43 (d, J=6.85 Hz, 1H), 7.05 (d, J=8.44 Hz, 1H), 3.87-3.93 (m, 3H), 3.61-3.66 (m, 2H), 3.56 (t, J=6.66 Hz, 2H), 3.32-3.32 (m, 3H), 3.32 (br. s., 3H), 3.28 (td, J=7.86, 3.36 Hz, 1H), 3.04 (t, J=6.66 Hz, 2H), 2.82 (br. s., 2H), 2.31 (br. s., 2H), 1.88-1.97 (m, 2H), 1.56-1.65 (m, 2H); LCMS: [M+H]⁺=509.51.

### Example 467: 6-(2-amino-5-(4-((1S,5R)-3-cyclobutyl-3-azabicyclo[3.1.0]hexan-1-yl)phenyl)-6-fluoropyridin-3-yl)-4-fluoroisoquinolin-1(2H)-one (I-520)

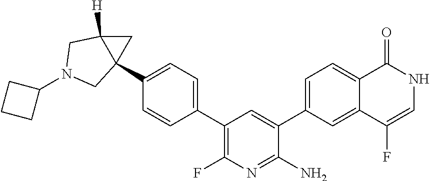 [SMILES: Nc1nc(F)c(-c2ccc([C@]34C[C@H]3CN(C3CCC3)C4)cc2)cc1-c1ccc2c(=O)[nH]cc(F)c2c1]

Prepared from 6-(2-amino-5-bromo-6-fluoropyridin-3-yl)-4-fluoroisoquinolin-1(2H)-one (40 mg, 0.114 mmol) and (1S,5R)-3-cyclobutyl-1-(4-(4,4,5,5-tetramethyl-1,3,2-dioxaborolan-2-yl)phenyl)-3-azabicyclo[3.1.0]hexane (38.5 mg, 0.114 mmol) to give the title compound (9.70 mg, 17.3% yield). ¹H NMR (500 MHz, METHANOL-d₄) 5=8.42 (dd, J=1.5, 8.4 Hz, 1H), 7.95 (d, J=1.2 Hz, 1H), 7.77 (dd, J=1.6, 8.3 Hz, 1H), 7.71 (d, J=9.8 Hz, 1H), 7.48 (br d, J=7.2 Hz, 2H), 7.27 (d, J=5.5 Hz, 1H), 7.24 (d, J=8.3 Hz, 2H), 3.37 (br d, J=9.3 Hz, 1H), 3.25-3.16 (m, 1H), 3.12 (br d, J=9.2 Hz, 1H), 2.80-2.65 (m, 2H), 2.13-1.96 (m, 4H), 1.86 (td, J=3.8, 7.9 Hz, 1H), 1.82-1.69 (m, 2H), 1.40 (t, J=4.5 Hz, 1H), 0.91 (br dd, J=4.7, 7.8 Hz, 1H); LCMS: [M+H]⁺=485.45.

### Example 468: 6-(2-amino-5-(3-((dimethylamino)methyl)-4-(tetrahydro-2H-pyran-4-yl)phenyl)-6-fluoropyridin-3-yl)-4-fluoro-3-methylisoquinolin-1(2H)-one (I-521)

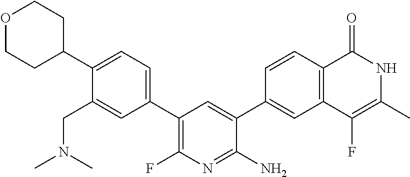 [SMILES: Cc1[nH]c(=O)c2ccc(-c3cc(-c4ccc(C5CCOCC5)c(CN(C)C)c4)c(F)nc3N)cc2c1F]

Prepared from 6-(2-amino-5-bromo-6-fluoropyridin-3-yl)-4-fluoro-3-methylisoquinolin-1(2H)-one (40 mg, 0.109 mmol) and N,N-dimethyl-1-(2-(tetrahydro-2H-pyran-4-yl)-5-(4,4,5,5-tetramethyl-1,3,2-dioxaborolan-2-yl)phenyl)methanamine (45.3 mg, 0.131 mmol) to give the title compound (9.4 mg, 17.1% yield) as a white solid. ¹H NMR (500 MHz, DMSO-d₆) δ ppm 11.26 (s, 1H), 8.22 (dd, J=8.25, 1.77 Hz, 1H), 7.75 (d, J=1.22 Hz, 1 H), 7.69 (d, J=10.03 Hz, 1H), 7.64 (dd, J=8.25, 1.53 Hz, 1H), 7.45 (d, J=7.82 Hz, 1H), 7.40 (br. s., 1H), 7.34 (d, J=8.19 Hz, 1H), 6.37 (s, 2H), 3.95 (dd, J=10.64, 3.55 Hz, 2H), 3.40-3.48 (m, 4H), 3.16-3.25 (m, 1H), 2.24 (d, J=3.06 Hz, 3H), 2.16 (br. s., 6H), 1.66-1.76 (m, 2H), 1.58-1.65 (m, 2H); LCMS [M+H]⁺=505.44.

### Example 469: 6-(2-amino-6-fluoro-5-(3-((3-methoxyazetidin-1-yl)methyl)-4-morpholinophenyl)pyridin-3-yl)-3,4-dihydroisoquinolin-1(2H)-one (I-522)

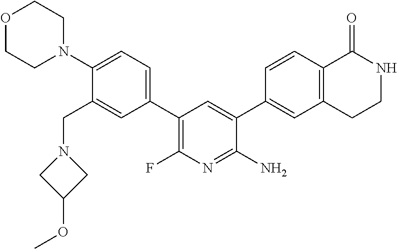 [SMILES: COC1CN(Cc2cc(-c3cc(-c4ccc5c(c4)CCNC5=O)c(N)nc3F)ccc2N2CCOCC2)C1]

Prepared from 4-(4-bromo-2-((3-methoxyazetidin-1-yl)methyl)phenyl)morpholine (55 mg, 0.153 mmol, in turn made from 5-bromo-2-morpholinobenzaldehyde and 3-methoxyazetidine) from and 6-(2-amino-6-fluoro-5-(4,4,5,5-tetramethyl-1,3,2-dioxaborolan-2-yl)pyridin-3-yl)-3,4-dihydroisoquinolin-1(2H)-one (76 mg, 0.199 mmol) to give the title compound as an off white solid (62 mg, 74%). ¹H NMR (500 MHz, DMSO-d₆) δ ppm 7.95 (br. s., 1H), 7.91 (d, J=7.95 Hz, 1H), 7.59 (d, J=10.15 Hz, 1H), 7.43-7.51 (m, 3H), 7.39 (d, J=8.31 Hz, 1H), 7.11 (d, J=8.31 Hz, 1H), 6.21-6.35 (m, 2H), 3.97 (quin, J=5.69 Hz, 1H), 3.72-3.80 (m, 4H), 3.66 (s, 2H), 3.50 (t, J=6.72 Hz, 2H), 3.40-3.43 (m, 2H), 3.14 (s, 3H), 2.96 (t, J=6.42 Hz, 2H), 2.86-2.93 (m, 6H); LCMS: [M+H]⁺=518.54.

### Example 470: 6-(2-amino-6-fluoro-5-(3-((3-methoxyazetidin-1-yl)methyl)-4-morpholinophenyl)pyridin-3-yl)-7-fluoro-3,4-dihydroisoquinolin-1(2H)-one (I-523)

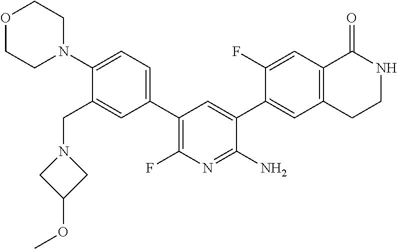 [SMILES: COC1CN(Cc2cc(-c3cc(-c4cc5c(cc4F)C(=O)NCC5)c(N)nc3F)ccc2N2CCOCC2)C1]

Prepared from 4-(4-bromo-2-((3-methoxyazetidin-1-yl)methyl)phenyl)morpholine (55 mg, 0.153 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (80 mg, 0.199 mmol) to yield the title compound as a white solid (25 mg, 29% yield). ¹H NMR (500 MHz, DMSO-d₆) δ ppm 8.12 (br. s., 1H), 7.61 (dd, J=12.23, 10.27 Hz, 2H), 7.47 (s, 1H), 7.43 (d, J=7.09 Hz, 1H), 7.33-7.40 (m, 1H), 7.37 (d, J=8.31 Hz, 1H), 7.06-7.15 (m, 1H), 6.27 (s, 2H), 3.96 (quin, J=5.69 Hz, 1H), 3.71-3.80 (m, 4H), 3.63-3.70 (m, 2H), 3.48-3.55 (m, 2H), 3.40-3.44 (m, 2H), 3.10-3.16 (m, 3H), 2.86-2.98 (m, 8H), 2.86-2.98 (m, 8H); LCMS: [M+H]⁺=536.59.

### Example 471: 6-(2-amino-5-(4-(4,4-difluorobutoxy)-3-((dimethylamino)methyl)phenyl)-6-uoropyridin-3-yl)-3,4-dihydroisoquinolin-1(2H)-one (I-524)

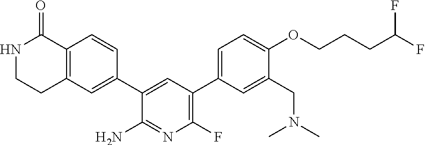 [SMILES: CN(C)Cc1cc(-c2cc(-c3ccc4c(c3)CCNC4=O)c(N)nc2F)ccc1OCCCC(F)F]

Step 1: 5-bromo-2-(4,4-difluorobutoxy)benzaldehyde

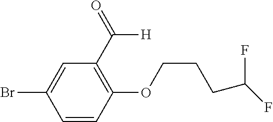 [SMILES: O=Cc1cc(Br)ccc1OCCCC(F)F]

A mixture of 5-bromosalicylaldehyde (0.2 g, 0.995 mmol), 4-bromo-1,1-difluorobutane (0.172 g, 0.995 mmol) and K₂CO₃ (0.275 g, 1.990 mmol) in DMF (4 ml) was stirred overnight at RT. The reaction mixture was partitioned between ethyl acetate (15 mL) and water (50 mL). The organic phase was separated, the aqueous layer was extracted with ethyl acetate (2×15 mL), the combined organic layers were washed with brine (2×50 mL), dried over Na₂SO₄ and evaporated in vacuo to get the desired product as a white solid (301 mg, 98%); LCMS: [M+H]⁺=293.14.

Step 2: 1-(5-bromo-2-(4,4-difluorobutoxy)phenyl)-N,N-dimethylmethanamine

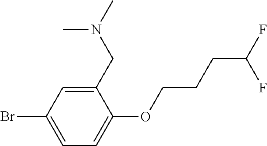 [SMILES: CN(C)Cc1cc(Br)ccc1OCCCC(F)F]

Prepared from 5-bromo-2-(4,4-difluorobutoxy)benzaldehyde (160 mg, 0.519 mmol) and dimethylamine to give the product (110 mg, 62.5% yield). LCMS: [M+H]⁺=322.29

Step 3: 6-(2-amino-5-(4-(4,4-difluorobutoxy)-3-((dimethylamino)methyl)phenyl)-6-fluoropyridin-3-yl)-3,4-dihydroisoquinolin-1(2H)-one

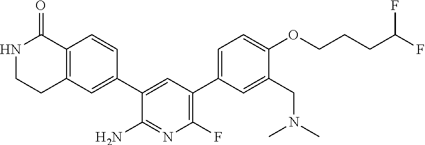 [SMILES: CN(C)Cc1cc(-c2cc(-c3ccc4c(c3)CCNC4=O)c(N)nc2F)ccc1OCCCC(F)F]

Prepared from 1-(5-bromo-2-(4,4-difluorobutoxy)phenyl)-N,N-dimethylmethanamine (55 mg, 0.162 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (81 mg, 0.211 mmol) to afford the title compound as a beige solid (51 mg, 60% yield). ¹H NMR (500 MHz, METHANOL-d₄) δ ppm 8.05 (d, J=7.95 Hz, 1H), 7.64-7.68 (m, 1H), 7.51 (dd, J=8.01, 1.65 Hz, 1H), 7.44-7.49 (m, 3H), 7.04 (d, J=8.19 Hz, 1H), 5.90-6.16 (m, 1H), 4.10 (t, J=5.99 Hz, 2H), 3.54-3.60 (m, 4H), 3.06 (t, J=6.66 Hz, 2H), 2.29-2.35 (m, 6H), 1.99-2.13 (m, 4H); LCMS: [M+H]⁺=499.55.

### Example 472: 6-(2-amino-5-(4-(4,4-difluorobutoxy)-3-((dimethylamino)methyl)phenyl)-6-fluoropyridin-3-yl)-7-fluoro-3,4-dihydroisoquinolin-1(2H)-one (I-525)

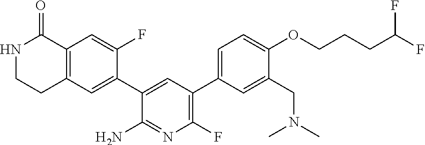 [SMILES: CN(C)Cc1cc(-c2cc(-c3cc4c(cc3F)C(=O)NCC4)c(N)nc2F)ccc1OCCCC(F)F]

Prepared from 1-(5-bromo-2-(4,4-difluorobutoxy)phenyl)-N,N-dimethylmethanamine (55 mg, 0.162 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (85 mg, 0.211 mmol) to afford the title compound as a beige solid (40 mg, 45% yield). ¹H NMR (500 MHz, METHANOL-d₄) δ ppm 7.74 (d, J=10.03 Hz, 1H), 7.64-7.68 (m, 1H), 7.52-7.58 (m, 2H), 7.41 (d, J=6.85 Hz, 1H), 7.09-7.16 (m, 1H), 5.91-6.17 (m, 1H), 4.16 (t, J=5.99 Hz, 2H), 3.90-4.03 (m, 2H), 3.53-3.60 (m, 2H), 3.03 (t, J=6.60 Hz, 2H), 2.54-2.67 (m, 6H), 2.01-2.15 (m, 4H); LCMS: [M+H]⁺=517.47

### Example 473: 6-(2-amino-5-(4-(4,4-difluorobutoxy)-3-((4-methoxypiperidin-1-yl)methyl)phenyl)-6-fluoropyridin-3-yl)-3,4-dihydroisoquinolin-1(2H)-one (I-526)

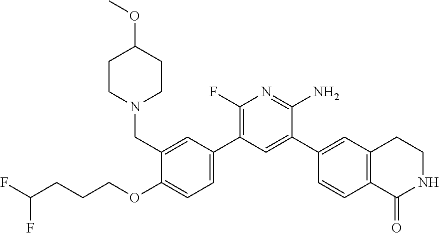 [SMILES: COC1CCN(Cc2cc(-c3cc(-c4ccc5c(c4)CCNC5=O)c(N)nc3F)ccc2OCCCC(F)F)CC1]

Prepared from 1-(5-bromo-2-(4,4-difluorobutoxy)benzyl)-4-methoxypiperidine (55 mg, 0.133 mmol, in turn made using 5-bromo-2-(4,4-difluorobutoxy)benzaldehyde and 4-methoxypiperidine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (66.4 mg, 0.173 mmol) to give the title compound as a white solid (62 mg, 78% yield). 1H NMR (500 MHz, METHANOL-d₄) δ ppm 8.03 (d, J=7.95 Hz, 1H), 7.62-7.67 (m, 1H), 7.41-7.52 (m, 4H), 7.00 (d, J=8.56 Hz, 1H), 5.89-6.15 (m, 1H), 4.08 (t, J=5.93 Hz, 2H), 3.59-3.65 (m, 2H), 3.54 (t, J=6.60 Hz, 2H), 3.31-3.33 (m, 3H), 3.21-3.28 (m, 1H), 3.05 (t, J=6.60 Hz, 2 H), 2.74-2.91 (m, 2H), 2.29 (t, J=8.93 Hz, 2H), 1.95-2.09 (m, 4H), 1.85-1.93 (m, 2H), 1.57 (d, J=9.29 Hz, 2H); LCMS: [M+H]⁺=569.61.

### Example 474: 6-(2-amino-5-(4-(4,4-difluorobutoxy)-3-((4-methoxypiperidin-1-yl)methyl)phenyl)-6-fluoropyridin-3-yl)-7-fluoro-3,4-dihydroisoquinolin-1(2H)-one (I-527)

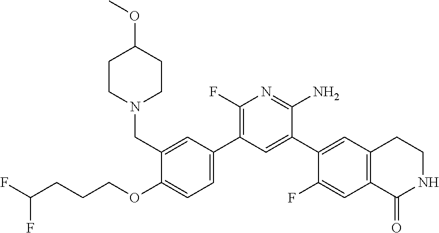 [SMILES: COC1CCN(Cc2cc(-c3cc(-c4cc5c(cc4F)C(=O)NCC5)c(N)nc3F)ccc2OCCCC(F)F)CC1]

Prepared from 1-(5-bromo-2-(4,4-difluorobutoxy)benzyl)-4-methoxypiperidine (55 mg, 0.133 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (69.5 mg, 0.173 mmol) to afford the title compound as an off white solid (31 mg, 38% yield). ¹H NMR (500 MHz, METHANOL-d₄) δ ppm 7.75 (d, J=10.03 Hz, 1H), 7.65 (d, J=8.80 Hz, 1H), 7.48 (s, 1H), 7.39-7.45 (m, 2H), 7.39-7.45 (m, 2 H), 7.01-7.05 (m, 1H), 5.91-6.17 (m, 1H), 4.10 (t, J=5.69 Hz, 2H), 3.62 (s, 2H), 3.56 (t, J=6.66 Hz, 2H), 3.32-3.33 (m, 3H), 3.23-3.30 (m, 1H), 3.03 (t, J=6.48 Hz, 2H), 2.83 (br. s., 2H), 2.26-2.37 (m, 2H), 1.98-2.13 (m, 4H), 1.91 (d, J=11.37 Hz, 2H), 1.59 (d, J=9.29 Hz, 2H); LCMS: [M+H]⁺=587.54

### Example 475: 6-(2-amino-5-(4-(3,6-dihydro-2H-pyran-4-yl)-2-(hydroxymethyl)phenyl)-6-fluoropyridin-3-yl)-7-fluoro-3,4-dihydroisoquinolin-1(2H)-one (I-528)

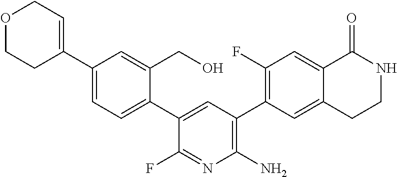 [SMILES: Nc1nc(F)c(-c2ccc(C3=CCOCC3)cc2CO)cc1-c1cc2c(cc1F)C(=O)NCC2]

To a solution of 2-(6-amino-2-fluoro-5-(7-fluoro-1-oxo-1,2,3,4-tetrahydroisoquinolin-6-yl)pyridin-3-yl)-5-(3,6-dihydro-2H-pyran-4-yl)benzaldehyde (70 mg, 0.152 mmol) in THF (3 ml) at r.t., NaBH₄ (57.4 mg, 1.517 mmol) was added. The reaction mixture was stirred for 5 min, quenched with MeOH (3 mL) and stirred at r.t. overnight. The reaction mixture was concentrated and the residue was diluted with water, the resulting precipitate was filtered, rinsed with water and ether to give the title compound (60 mg, 84% yield). ¹H NMR (500 MHz, METHANOL-d₄) δ=7.75 (d, J=10.0 Hz, 1H), 7.69 (d, J=1.7 Hz, 1H), 7.50 (d, J=9.4 Hz, 1H), 7.46-7.40 (m, 2H), 7.25 (d, J=7.9 Hz, 1H), 6.29 (td, J=1.4, 2.9 Hz, 1H), 4.56 (s, 2H), 4.34 (q, J=2.7 Hz, 2H), 3.97 (t, J=5.5 Hz, 2H), 3.55 (t, J=6.7 Hz, 2H), 3.03 (t, J=6.6 Hz, 2H), 2.62-2.56 (m, 2H); [M+H]⁺=464.45.

### Example 476: 6-(2-amino-5-(4-(4-(cyclopropylmethyl)piperazin-1-yl)phenyl)-6-fluoropyridin-3-yl)-4,8-difluoro-3-methylisoquinolin-1(2H)-one (l-529)

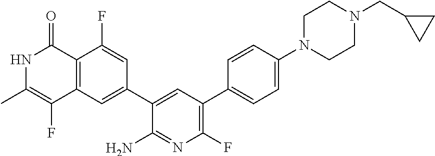 [SMILES: Cc1[nH]c(=O)c2c(F)cc(-c3cc(-c4ccc(N5CCN(CC6CC6)CC5)cc4)c(F)nc3N)cc2c1F]

Step 1: 6-(2-amino-5-bromo-6-fluoropyridin-3-yl)-4,8-difluoro-3-methylisoquinolin-1(2H)-one

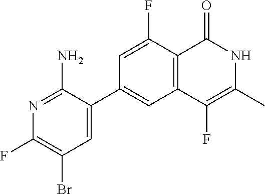 [SMILES: Cc1[nH]c(=O)c2c(F)cc(-c3cc(Br)c(F)nc3N)cc2c1F]

Prepared from 5-bromo-6-fluoro-3-iodopyridin-2-amine (220 mg, 0.694 mmol) and (4,8-difluoro-3-methyl-1-oxo-1,2-dihydroisoquinolin-6-yl)boronic acid (199 mg, 0.833 mmol) to give the product (187 mg, 70% yield) as a grey solid. ¹H NMR (500 MHz, DMSO-d₆) δ ppm 11.27 (br. s., 1H), 7.82 (d, J=8.93 Hz, 1H), 7.46 (d, J=0.98 Hz, 1H), 7.31 (d, J=12.47 Hz, 1H), 6.58 (s, 2H), 2.22 (d, J=3.06 Hz, 3H); LCMS: [M+H]⁺=384.22.

Step 2: 6-(2-amino-5-(4-(4-(cyclopropylmethyl)piperazin-1-yl)phenyl)-6-fluoropyridin-3-yl)-4,8-difluoro-3-methylisoquinolin-1(2H)-one

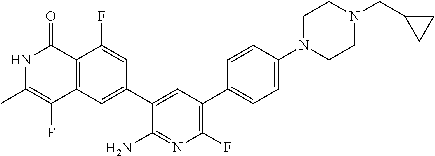 [SMILES: Cc1[nH]c(=O)c2c(F)cc(-c3cc(-c4ccc(N5CCN(CC6CC6)CC5)cc4)c(F)nc3N)cc2c1F]

Prepared from 6-(2-amino-5-bromo-6-fluoropyridin-3-yl)-4,8-difluoro-3-methylisoquinolin-1(2H)-one (35 mg, 0.091 mmol) and 1-(cyclopropylmethyl)-4-(4-(4,4,5,5-tetramethyl-1,3,2-dioxaborolan-2-yl)phenyl)piperazine (37.4 mg, 0.109 mmol) to give the title compound (16.3 mg, 34.4% yield) as a pale yellow solid. 1H NMR (500 MHz, DMSO-d₆) b ppm 11.25 (br. s., 1H), 7.69 (d, J=10.15 Hz, 1H), 7.53 (d, J=0.98 Hz, 1H), 7.37-7.45 (m, 3 H), 6.98 (d, J=8.93 Hz, 2H), 6.36 (s, 2H), 3.14-3.20 (m, 4H), 2.56-2.59 (m, 4H), 2.20-2.25 (m, 5H), 0.82-0.89 (m, 1H), 0.46-0.50 (m, 2H), 0.08-0.12 (m, 2H); LCMS: [M+H]⁺=520.54.

### Example 477: 6-(2-amino-5-(3-((dimethylamino)methyl)-4-((tetrahydro-2H-pyran-4-yl)oxy)phenyl)-6-fluoropyridin-3-yl)-3,4-dihydroisoquinolin-1(2H)-one (I-530)

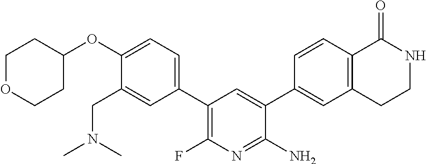 [SMILES: CN(C)Cc1cc(-c2cc(-c3ccc4c(c3)CCNC4=O)c(N)nc2F)ccc1OC1CCOCC1]

Step 1: 1-(5-bromo-2-(cyclopropylmethoxy)benzyl)-4-methoxypiperidine

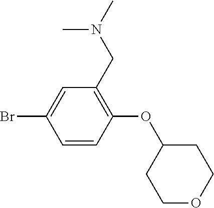 [SMILES: CN(C)Cc1cc(Br)ccc1OC1CCOCC1]

Prepared from 5-bromo-2-((tetrahydro-2H-pyran-4-yl)oxy)benzaldehyde (150 mg, 0.526 mmol) and dimethylamine, 2.0M solution in THF (1.052 ml, 2.104 mmol) to give the product (169 mg, 97% yield). LCMS: [M+H]⁺=314.26.

Step 2: 6-(2-amino-5-(3-((dimethylamino)methyl)-4-((tetrahydro-2H-pyran-4-yl)oxy)phenyl)-6-fluoropyridin-3-yl)-3,4-dihydroisoquinolin-1(2H)-one

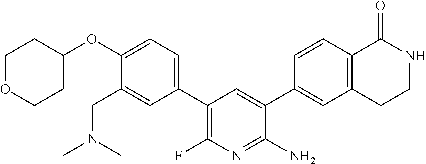 [SMILES: CN(C)Cc1cc(-c2cc(-c3ccc4c(c3)CCNC4=O)c(N)nc2F)ccc1OC1CCOCC1]

Prepared from 1-(5-bromo-2-((tetrahydro-2H-pyran-4-yl)oxy)phenyl)-N,N-dimethylmethanamine (55 mg, 0.175 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (87 mg, 0.228 mmol) to afford the title compound as a beige solid (63 mg, 70% yield). ¹H NMR (500 MHz, DMSO-d₆) δ ppm 7.94 (br. s., 1H), 7.91 (d, J=7.95 Hz, 1H), 7.56-7.61 (m, 1H), 7.43-7.48 (m, 3 H), 7.38 (d, J=8.68 Hz, 1H), 7.05-7.12 (m, 1H), 6.22 (s, 2H), 4.64 (tt, J=7.64, 3.73 Hz, 1 H), 3.81-3.90 (m, 2H), 3.52 (ddd, J=11.40, 8.28, 3.06 Hz, 2H), 3.43 (s, 2H), 3.39-3.42 (m, 2H), 3.40 (dd, J=6.54, 2.51 Hz, 2H), 2.96 (t, J=6.54 Hz, 2H), 2.15-2.23 (m, 6H), 1.93-2.00 (m, 2H), 1.59-1.69 (m, 2H); LCMS: [M+H]⁺=491.59.

### Example 478: 6-(2-amino-5-(3-((dimethylamino)methyl)-4-((tetrahydro-2H-pyran-4-yl)oxy)phenyl)-6-fluoropyridin-3-yl)-7-fluoro-3,4-dihydroisoquinolin-1(2H)-one (I-531)

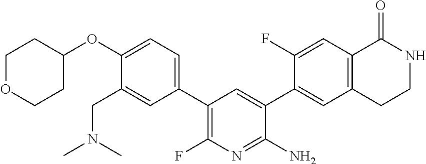 [SMILES: CN(C)Cc1cc(-c2cc(-c3cc4c(cc3F)C(=O)NCC4)c(N)nc2F)ccc1OC1CCOCC1]

Prepared from (5-bromo-2-((tetrahydro-2H-pyran-4-yl)oxy)phenyl)-N,N-dimethylmethanamine (55 mg, 0.175 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (91 mg, 0.228 mmol) to afford the title compound as an off white solid (31 mg, 33% yield). ¹H NMR (500 MHz, DMSO-d₆) δ ppm 8.11 (br. s., 1H), 7.60 (dd, J=18.77, 10.09 Hz, 2H), 7.41-7.47 (m, 2H), 7.36 (d, J=8.44 Hz, 1H), 7.08 (d, J=8.68 Hz, 1H), 6.23 (s, 2H), 4.63 (dt, J=7.55, 3.87 Hz, 1H), 3.81-3.87 (m, 2H), 3.51-3.56 (m, 2H), 3.43 (s, 2H), 3.40-3.41 (m, 2H), 2.92 (t, J=6.36 Hz, 2H), 2.18 (s, 6H), 1.93-2.00 (m, 2H), 1.59-1.68 (m, 2H); LCMS: [M+H]⁺=509.51.

### Example 479: 6-(2-amino-5-(3-((dimethylamino)methyl)-4-((tetrahydro-2H-pyran-4-yl)methoxy)phenyl)-6-fluoropyridin-3-yl)-3,4-dihydroisoquinolin-1(2H)-one (I-532)

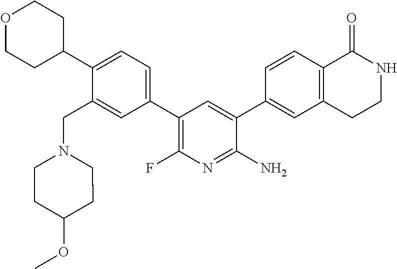 [SMILES: COC1CCN(Cc2cc(-c3cc(-c4ccc5c(c4)CCNC5=O)c(N)nc3F)ccc2C2CCOCC2)CC1]

Prepared from 1-(5-bromo-2-((tetrahydro-2H-pyran-4-yl)methoxy)phenyl)-N,N-dimethylmethanamine (80 mg, 0.171 mmol, in turn made from 5-bromo-2-((tetrahydro-2H-pyran-4-yl)methoxy)benzaldehyde and dimethylamine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (85 mg, 0.222 mmol) to afford the title compound as an off white solid (67.5 mg, 75% yield). ¹H NMR (500 MHz, DMSO-d₆) δ ppm 7.95 (br. s., 1H), 7.91 (d, J=7.95 Hz, 1H), 7.57 (d, J=10.03 Hz, 1H), 7.42-7.49 (m, 3H), 7.39 (d, J=8.44 Hz, 1H), 7.01 (d, J=8.44 Hz, 1H), 6.22 (br. s., 2H), 3.88-3.93 (m, 2H), 3.86 (d, J=5.99 Hz, 2H), 3.42 (br. s., 4H), 3.38 (br. s., 2H), 2.96 (t, J=6.36 Hz, 2H), 2.17 (s, 6H), 2.03 (d, J=4.65 Hz, 1H), 1.70 (d, J=12.59 Hz, 2 H), 1.39 (qd, J=12.17, 4.34 Hz, 2H); LCMS: [M+H]⁺=505.50.

### Example 480 6-(2-amino-5-(3-((dimethylamino)methyl)-4-((tetrahydro-2H-pyran-4-yl)methoxy)phenyl)-6-fluoropyridin-3-yl)-7-fluoro-3,4-dihydroisoquinolin-1(2H)-one (I-533)

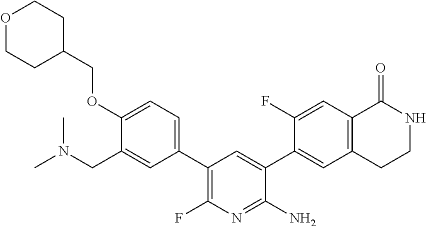 [SMILES: CN(C)Cc1cc(-c2cc(-c3cc4c(cc3F)C(=O)NCC4)c(N)nc2F)ccc1OCC1CCOCC1]

Prepared from 1-(5-bromo-2-((tetrahydro-2H-pyran-4-yl)methoxy)phenyl)-N,N-dimethylmethanamine (80 mg, 0.171 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (89 mg, 0.222 mmol) to afford the title compound as a white solid (29 mg, 31% yield). ¹H NMR (500 MHz, DMSO-d₆) δ ppm 8.11 (br. s., 1H), 7.56-7.65 (m, 2H), 7.44 (br. s., 2H), 7.37 (d, J=8.44 Hz, 1H), 7.01 (d, J=8.44 Hz, 1H), 6.23 (br. s., 2H), 3.90 (d, J=9.54 Hz, 2H), 3.86 (d, J=5.75 Hz, 2H), 3.41 (br. s., 4H), 2.93 (br. s., 2H), 2.17 (s, 6H), 2.02 (br. s., 1H), 1.70 (d, J=12.72 Hz, 2H), 1.33-1.44 (m, 2H); LCMS: [M+H]⁺=523.61

### Example 481: 6-(2-amino-5-(3-((dimethylamino)methyl)-4-morpholinophenyl)-6-fluoropyridin-3-yl)-4,8-difluoro-3-methylisoquinolin-1(2H)-one (I-534)

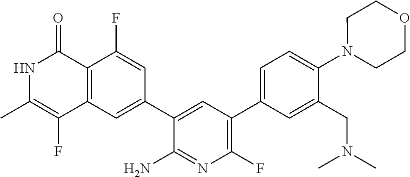 [SMILES: Cc1[nH]c(=O)c2c(F)cc(-c3cc(-c4ccc(N5CCOCC5)c(CN(C)C)c4)c(F)nc3N)cc2c1F]

Prepared from 6-(2-amino-5-bromo-6-fluoropyridin-3-yl)-4,8-difluoro-3-methylisoquinolin-1(2H)-one (35 mg, 0.091 mmol) and N,N-dimethyl-1-(2-morpholino-5-(4,4,5,5-tetramethyl-1,3,2-dioxaborolan-2-yl)phenyl)methanamine (37.9 mg, 0.109 mmol) to give the title compound (5.7 mg, 12% yield) as a beige solid. ¹H NMR (500 MHz, DMSO-d₆) b ppm 11.25 (br. s., 1H), 7.69 (d, J=10.15 Hz, 1H), 7.53 (s, 2H), 7.37-7.45 (m, 2H), 7.13 (d, J=8.31 Hz, 1H), 6.43 (s, 2H), 3.71-3.77 (m, 4H), 3.44 (s, 2H), 2.91-2.97 (m, 4H), 2.22 (d, J=2.81 Hz, 3H), 2.18 (s, 6H); LCMS: [M+H]⁺=524.46.

### Example 482: 6-(2-amino-5-(3-((dimethylamino)methyl)-4-(tetrahydro-2H-pyran-4-yl)phenyl)-6-fluoropyridin-3-yl)-4,8-difluoro-3-methylisoquinolin-1(2H)-one (I-535)

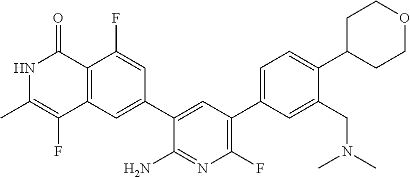 [SMILES: Cc1[nH]c(=O)c2c(F)cc(-c3cc(-c4ccc(C5CCOCC5)c(CN(C)C)c4)c(F)nc3N)cc2c1F]

Prepared from 6-(2-amino-5-bromo-6-fluoropyridin-3-yl)-4,8-difluoro-3-methylisoquinolin-1(2H)-one (35 mg, 0.091 mmol) and N,N-dimethyl-1-(2-(tetrahydro-2H-pyran-4-yl)-5-(4,4,5,5-tetramethyl-1,3,2-dioxaborolan-2-yl)phenyl)methanamine (37.8 mg, 0.109 mmol) to give the title compound (15.8 mg, 33.2% yield) as a beige solid. 1H NMR (500 MHz, DMSO-d₆) δ ppm 11.26 (br. s., 1H), 7.73 (d, J=10.03 Hz, 1H), 7.59-7.64 (m, 1 H), 7.54-7.58 (m, 1H), 7.53 (s, 1H), 7.34-7.41 (m, 2H), 6.47 (br. s., 2H), 3.95 (dd, J=10.70, 3.24 Hz, 2H), 3.44 (t, J=10.58 Hz, 4H), 3.14-3.24 (m, 1H), 2.22 (d, J=2.81 Hz, 3H), 2.17 (br. s., 6H), 1.66-1.76 (m, 2H), 1.57-1.65 (m, 2H); LCMS: [M+H]⁺=523.41.

### Example 483: 6-(2-amino-6-fluoro-5-(3-(hydroxymethyl)-4-methoxyphenyl)pyridin-3-yl)-3,4-dihydroisoquinolin-1(2H)-one (I-536)

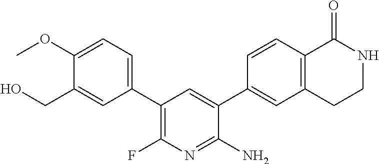 [SMILES: COc1ccc(-c2cc(-c3ccc4c(c3)CCNC4=O)c(N)nc2F)cc1CO]

Prepared from 5-bromo-2-methoxyphenyl)methanol (60 mg, 0.235 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (117 mg, 0.305 mmol) to afford the title compound as an off white solid (74.5 mg, 77% yield). 1H NMR ((500 MHz, METHANOL-d₄) δ=8.10-8.01 (m, 1H), 7.69-7.62 (m, 1H), 7.59-7.55 (m, 1H), 7.54-7.50 (m, 1H), 7.49-7.46 (m, 1H), 7.46-7.42 (m, 1H), 7.06-6.96 (m, 1H), 4.72-4.67 (m, 2H), 3.92-3.86 (m, 3H), 3.60-3.53 (m, 2H), 3.11-3.05 (m, 2H); LCMS: [M+H]⁺=394.37.

### Example 484: 6-(2-amino-6-fluoro-5-(4-(4-isopropylpiperazin-1-yl)phenyl)pyridin-3-yl)-4-ethoxyisoquinolin-1(2H)-one (I-537)

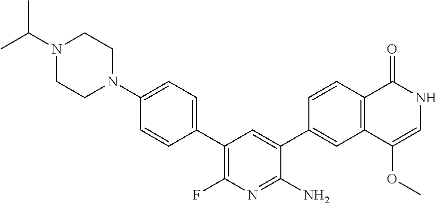 [SMILES: COc1c[nH]c(=O)c2ccc(-c3cc(-c4ccc(N5CCN(C(C)C)CC5)cc4)c(F)nc3N)cc12]

Step 1: 6-bromo-4-methoxyisoquinolin-1(2H)-one

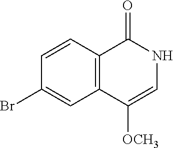 [SMILES: COc1c[nH]c(=O)c2ccc(Br)cc12]

A vial was charged with 6-bromo-2H-isoquinolin-1-one (300 mg, 1.339 mmol), methanol (2 ml) and methanesulfonic acid (0.087 ml, 1.339 mmol) then cooled to 0° C. A solution of iodobenzene diacetate (474 mg, 1.473 mmol) in methanol (2 ml) was added slowly. The reaction was warmed to room temperature and stirred for 1 hour then heated at 50° C. overnight. The reaction was concentrated in vacuo and triturated with isopropanol. The filtered solid was vacuum dried to afford 6-bromo-4-methoxyisoquinolin-1(2H)-one (257 mg, 1.011 mmol, 76% yield) as a pink solid. LCMS [M+H]⁺=254.20.

Step 2: 4-methoxy-6-(4,4,5,5-tetramethyl-1,3,2-dioxaborolan-2-yl)isoquinolin-1(2H)-one

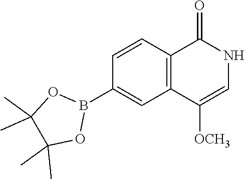 [SMILES: COc1c[nH]c(=O)c2ccc(B3OC(C)(C)C(C)(C)O3)cc12]

Prepared from 6-bromo-4-methoxyisoquinolin-1(2H)-one (70 mg, 0.276 mmol) to give the boronate which was used in the next step without further purification. LCMS: [M+H]⁺=302.23.

Step 3: 6-(2-amino-6-fluoro-5-(4-(4-isopropylpiperazin-1-yl)phenyl)pyridin-3-yl)-4-methoxyisoquinolin-1(2H)-one

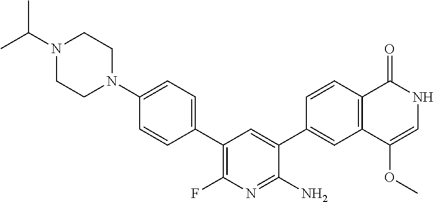 [SMILES: COc1c[nH]c(=O)c2ccc(-c3cc(-c4ccc(N5CCN(C(C)C)CC5)cc4)c(F)nc3N)cc12]

Prepared from 4-methoxy-6-(4,4,5,5-tetramethyl-1,3,2-dioxaborolan-2-yl)isoquinolin-1(2H)-one (36.8 mg, 0.122 mmol) and 3-bromo-6-fluoro-5-(4-(4-isopropylpiperazin-1-yl)phenyl)pyridin-2-amine (40 mg, 0.102 mmol) to give the title compound (23.8 mg, 48.0% yield) as a beige solid. ¹H NMR (500 MHz, DMSO-d₆) δ ppm 10.95 (d, J=4.89 Hz, 1H), 8.25 (d, J=8.31 Hz, 1H), 7.90 (d, J=1.22 Hz, 1H), 7.67 (dd, J=8.25, 1.53 Hz, 1H), 7.62 (d, J=10.15 Hz, 1H), 7.40 (d, J=7.95 Hz, 2H), 6.96 (d, J=8.80 Hz, 2H), 6.77 (d, J=5.38 Hz, 1H), 6.20 (s, 2H), 3.79 (s, 3H), 3.12-3.17 (m, 4H), 2.67 (dt, J=13.11, 6.59 Hz, 1H), 2.56-2.59 (m, 4H), 1.00 (d, J=6.48 Hz, 6H); LCMS: [M+H]⁺=488.50.

### Example 485: 6-(2-amino-6-fluoro-5-(4-(4-isopropylpiperazin-1-yl)phenyl)pyridin-3-yl)-4-chloro-3-methylisoquinolin-1(2H)-one (I-538)

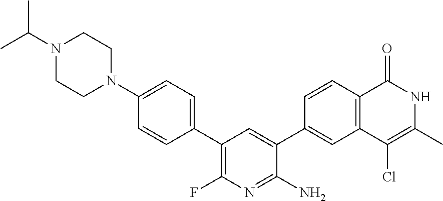 [SMILES: Cc1[nH]c(=O)c2ccc(-c3cc(-c4ccc(N5CCN(C(C)C)CC5)cc4)c(F)nc3N)cc2c1Cl]

Step 1: 6-bromo-4-chloro-3-methylisoquinolin-1(2H)-one

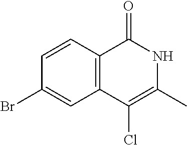

To a solution of 6-bromo-3-methyl-2H-isoquinolin-1-one (1.0 g, 4.20 mmol) in N,N-dimethylacetamide (14 ml) was added N-chlorosuccinimide (0.673 g, 5.04 mmol). The mixture was stirred at 50° C. for 8 hours. The reaction was cooled to RT and diluted with water. The suspension was filtered and washed with water. The solid was further triturated from MeOH and dried under vacuum to give the product (1.12 g, 98% yield) as a white solid. LCMS: [M+H]⁺=272.20.

Step 2: (4-chloro-3-methyl-1-oxo-1,2-dihydroisoquinolin-6-yl)boronic acid

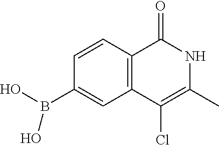 [SMILES: Cc1[nH]c(=O)c2ccc(B(O)O)cc2c1Cl]

Prepared from 6-bromo-4-chloro-3-methylisoquinolin-1(2H)-one (1.1 g, 4.04 mmol) to give a mixture of boronate and boronic acid which was used in the next step without further purification. LCMS: [M+H]⁺=238.34 Boronic acid, 320.34 Boronate.

Step 3: 6-(2-amino-5-bromo-6-fluoropyridin-3-yl)-4-chloro-3-methylisoquinolin-1(2H)-one

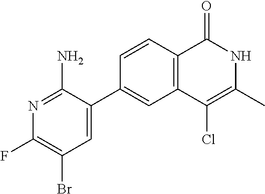 [SMILES: Cc1[nH]c(=O)c2ccc(-c3cc(Br)c(F)nc3N)cc2c1Cl]

Prepared from 5-bromo-6-fluoro-3-iodopyridin-2-amine (425 mg, 1.341 mmol) and (4-chloro-3-methyl-1-oxo-1,2-dihydroisoquinolin-6-yl)boronic acid (446 mg, 1.878 mmol) to give the product (112 mg, 21.8% yield) as a beige solid. LCMS [M+H]⁺=382.21.

Step 4: 6-(2-amino-6-fluoro-5-(4-(4-isopropylpiperazin-1-yl)phenyl)pyridin-3-yl)-4-chloro-3-methylisoquinolin-1(2H)-one

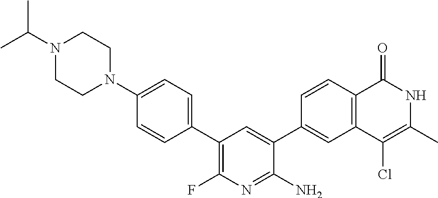 [SMILES: Cc1[nH]c(=O)c2ccc(-c3cc(-c4ccc(N5CCN(C(C)C)CC5)cc4)c(F)nc3N)cc2c1Cl]

Prepared from 6-(2-amino-5-bromo-6-fluoropyridin-3-yl)-4-chloro-3-methylisoquinolin-1(2H)-one (30 mg, 0.078 mmol) and 4-(4-isopropylpiperazinyl)phenylboronic acid, pinacol ester (31.1 mg, 0.094 mmol) to give the title compound (18.3 mg, 46% yield) as a pale yellow solid. ¹H NMR (500 MHz, DMSO-d₆) δ ppm 11.67 (br. s., 1H), 8.25 (d, J=8.19 Hz, 1H), 7.87 (s, 1H), 7.62-7.67 (m, 2H), 7.40 (d, J=8.07 Hz, 2H), 6.97 (d, J=8.80 Hz, 2H), 6.24 (s, 2H), 3.12-3.17 (m, 4H), 2.64-2.70 (m, 1H), 2.55-2.59 (m, 4H), 2.38 (s, 3H), 1.00 (d, J=6.60 Hz, 6H); LCMS: [M+H]⁺=506.56.

### Example 486: 6-(2-amino-6-fluoro-5-(4-(8-methyl-5-oxa-2,8-diazaspiro[3.5]nonan-2-yl)phenyl)pyridin-3-yl)-7-fluoro-3,4-dihydroisoquinolin-1(2H)-one (I-539)

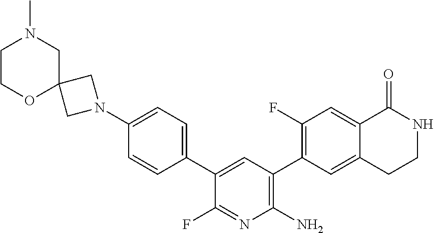 [SMILES: CN1CCOC2(C1)CN(c1ccc(-c3cc(-c4cc5c(cc4F)C(=O)NCC5)c(N)nc3F)cc1)C2]

Prepared from 6-(2-amino-5-bromo-6-fluoropyridin-3-yl)-7-fluoro-3,4-dihydroisoquinolin-1(2H)-one (30.0 mg, 0.085 mmol) and 8-methyl-2-(4-(4,4,5,5-tetramethyl-1,3,2-dioxaborolan-2-yl)phenyl)-5-oxa-2,8-diazaspiro[3.5]nonane (40 mg, 0.085 mmol) to give the title compound (12.3 mg, 29.9% yield). ¹H NMR (500 MHz, METHANOL-d₄) δ=7.75 (d, J=10.0 Hz, 1H), 7.59 (d, J=9.8 Hz, 1H), 7.41 (d, J=6.8 Hz, 1H), 7.38 (d, J=8.2 Hz, 2H), 6.58 (d, J=8.4 Hz, 2H), 3.91 (d, J=7.9 Hz, 2H), 3.76 (t, J=4.6 Hz, 2H), 3.70 (d, J=7.8 Hz, 2H), 3.56 (t, J=6.7 Hz, 2H), 3.03 (t, J=6.6 Hz, 2H), 2.70 (br s, 2H), 2.48 (br s, 2H), 2.38 (s, 3H); LCMS: [M+H]⁺=492.46;

### Example 487: 6-(2-amino-6-fluoro-5-(3-((4-methoxypiperidin-1-yl)methyl)-4-(tetrahydro-2H-pyran-4-yl)phenyl)pyridin-3-yl)-3,4-dihydroisoquinolin-1(2H)-one (I-540)

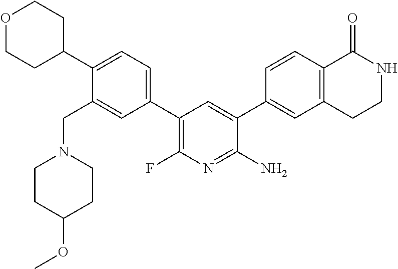 [SMILES: COC1CCN(Cc2cc(-c3cc(-c4ccc5c(c4)CCNC5=O)c(N)nc3F)ccc2C2CCOCC2)CC1]

Prepared from 1-(5-bromo-2-(tetrahydro-2H-pyran-4-yl)benzyl)-4-methoxypiperidine (55 mg, 0.142 mmol, in turn made from 5-bromo-2-(tetrahydro-2H-pyran-4-yl)benzaldehyde and 4-methoxypiperidine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (70.7 mg, 0.184 mmol) to give the title compound as a beige solid (27 mg, 33% yield). ¹H NMR (500 MHz, DMSO-d₆) δ ppm 7.95 (br. s., 1H), 7.91 (d, J=7.95 Hz, 1H), 7.63 (d, J=10.03 Hz, 1H), 7.39-7.50 (m, 3H), 7.31-7.38 (m, 2H), 6.29 (s, 2H), 3.92-4.02 (m, 2H), 3.50 (s, 2H), 3.39-3.46 (m, 4H), 3.14-3.24 (m, 5H), 2.95 (t, J=6.42 Hz, 2H), 2.64 (br. s., 2H), 2.10 (t, J=9.48 Hz, 2H), 1.75-1.85 (m, 2H), 1.67-1.75 (m, 2H), 1.59-1.66 (m, 2H), 1.33 (d, J=9.41 Hz, 2H); LCMS: [M+H]⁺=545.53.

### Example 488: 6-(2-amino-6-fluoro-5-(3-((4-methoxypiperidin-1-yl)methyl)-4-(tetrahydro-2H-pyran-4-yl)phenyl)pyridin-3-yl)-7-fluoro-3,4-dihydroisoquinolin-1(2H)-one (I-541)

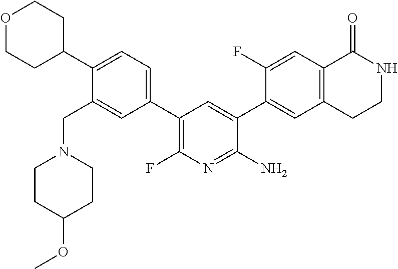 [SMILES: COC1CCN(Cc2cc(-c3cc(-c4cc5c(cc4F)C(=O)NCC5)c(N)nc3F)ccc2C2CCOCC2)CC1]

Prepared from 1-(5-bromo-2-(tetrahydro-2H-pyran-4-yl)benzyl)-4-methoxypiperidine (55 mg, 0.142 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (74 mg, 0.184 mmol) to give the title compound as a white solid (23 mg, 27% yield). ¹H NMR (500 MHz, DMSO-d₆ b ppm 8.12 (br. s., 1H), 7.58-7.67 (m, 2H), 7.42 (t, J=7.58 Hz, 2H), 7.34 (d, J=7.34 Hz, 2H), 6.29 (s, 2H), 3.92-4.03 (m, 2H), 3.49 (s, 2H), 3.39-3.46 (m, 4H), 3.39-3.46 (m, 4H), 3.13-3.22 (m, 5H), 2.90-2.97 (m, 2H), 2.64 (br. s., 2H), 2.10 (t, J=8.93 Hz, 2H), 1.79 (d, J=9.90 Hz, 2H), 1.68-1.76 (m, 2H), 1.61-1.67 (m, 2H), 1.32 (d, J=8.80 Hz, 2H); LCMS: [M+H]⁺=563.60.

### Example 489: 6-(2-amino-6-fluoro-5-(3-((3-methoxypyrrolidin-1-yl)methyl)-4-morpholinophenyl)pyridin-3-yl)-3,4-dihydroisoquinolin-1(2H)-one (I-542)

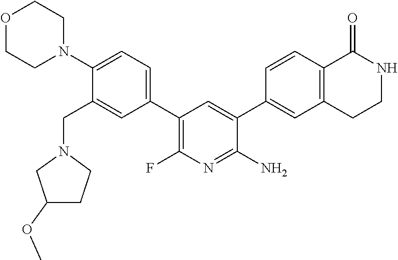 [SMILES: COC1CCN(Cc2cc(-c3cc(-c4ccc5c(c4)CCNC5=O)c(N)nc3F)ccc2N2CCOCC2)C1]

Prepared from 4-(4-bromo-2-((3-methoxypyrrolidin-1-yl)methyl)phenyl)morpholine (60 mg, 0.169 mmol, in turn made from 5-bromo-2-morpholinobenzaldehyde and 3-methoxypyrrolidine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (84 mg, 0.220 mmol) to give the title compound as a white solid (48 mg, 51% yield). ¹H NMR (500 MHz, DMSO-d₆) δ ppm 5 7.9-8.0 (m, 1H), 7.91 (d, 1H, J=7.9 Hz), 7.6-7.6 (m, 1H), 7.5-7.5 (m, 1H), 7.4-7.5 (m, 2H), 7.4-7.4 (m, 1H), 7.1-7.2 (m, 1H), 6.2-6.3 (m, 2H), 3.8-3.9 (m, 1H), 3.7-3.8 (m, 4H), 3.6-3.7 (m, 2H), 3.4-3.4 (m, 2H), 3.1-3.2 (m, 3H), 2.9-3.0 (m, 6H), 2.7-2.7 (m, 1H), 2.6-2.7 (m, 1H), 2.55 (br d, 1H, J=1.7 Hz), 2.4-2.5 (m, 1H), 1.9-2.0 (m, 1H), 1.6-1.7 (m, 1H); LCMS: [M+H]⁺=532.58.

### Example 490: 6-(2-amino-6-fluoro-5-(3-((3-methoxypyrrolidin-1-yl)methyl)-4-morpholinophenyl)pyridin-3-yl)-7-fluoro-3,4-dihydroisoquinolin-1(2H)-one (I-543)

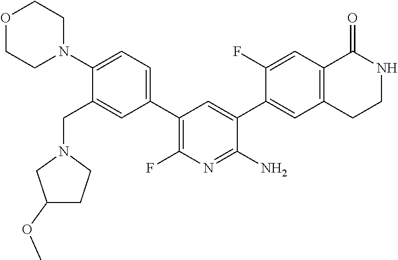 [SMILES: COC1CCN(Cc2cc(-c3cc(-c4cc5c(cc4F)C(=O)NCC5)c(N)nc3F)ccc2N2CCOCC2)C1]

Prepared from 4-(4-bromo-2-((3-methoxypyrrolidin-1-yl)methyl)phenyl)morpholine (60 mg, 0.169 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (84 mg, 0.220 mmol) to give the title compound as a white solid (48 mg, 51% yield). 1H NMR (500 MHz, DMSO-d₆) δ ppm 5 7.9-8.0 (m, 1H), 7.91 (d, 1H, J=7.9 Hz), 7.6-7.6 (m, 1H), 7.5-7.5 (m, 1H), 7.4-7.5 (m, 2H), 7.4-7.4 (m, 1H), 7.1-7.2 (m, 1H), 6.2-6.3 (m, 2H), 3.8-3.9 (m, 1H), 3.7-3.8 (m, 4H), 3.6-3.7 (m, 2H), 3.4-3.4 (m, 2H), 3.1-3.2 (m, 3H), 2.9-3.0 (m, 6H), 2.7-2.7 (m, 1H), 2.6-2.7 (m, 1H), 2.55 (br d, 1H, J=1.7 Hz), 2.4-2.5 (m, 1H), 1.9-2.0 (m, 1H), 1.6-1.7 (m, 1H); LCMS: [M+H]⁺=532.58.

### Example 491 6-(2-amino-5-(4-(4-(4,4-difluorobutyl)piperazin-1-yl)phenyl)-6-fluoropyridin-3-yl)-4-fluoroisoquinolin-1(2H)-one (I-544))

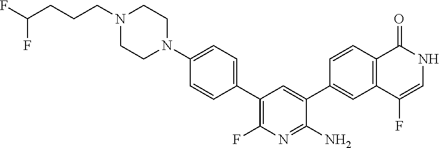 [SMILES: Nc1nc(F)c(-c2ccc(N3CCN(CCCC(F)F)CC3)cc2)cc1-c1ccc2c(=O)[nH]cc(F)c2c1]

Step 1: 6-(2-amino-6-fluoro-5-(4-(piperazin-1-yl)phenyl)pyridin-3-yl)-4-fluoroisoquinolin-1(2H)-one

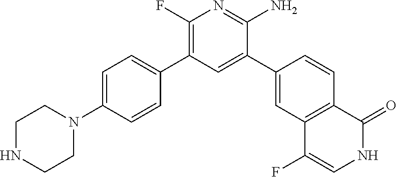 [SMILES: Nc1nc(F)c(-c2ccc(N3CCNCC3)cc2)cc1-c1ccc2c(=O)[nH]cc(F)c2c1]

Prepared from 6-(2-amino-5-bromo-6-fluoropyridin-3-yl)-4-fluoroisoquinolin-1(2H)-one (50 mg, 0.142 mmol), and 4-piperazinylphenylboronic acid, pinacol ester (53.2 mg, 0.185 mmol) to give the title compound as a beige solid (30 mg, 49%); LCMS: [M+H]⁺=434.43.

Step 2: 6-(2-amino-5-(4-(4-(4,4-difluorobutyl)piperazin-1-yl)phenyl)-6-fluoropyridin-3-yl)-4-fluoroisoquinolin-1(2H)-one

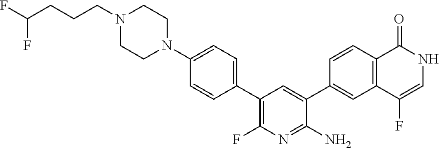 [SMILES: Nc1nc(F)c(-c2ccc(N3CCN(CCCC(F)F)CC3)cc2)cc1-c1ccc2c(=O)[nH]cc(F)c2c1]

Prepared from 6-(2-amino-6-fluoro-5-(4-(piperazin-1-yl)phenyl)pyridin-3-yl)-4-fluoroisoquinolin-1(2H)-one (30 mg, 0.069 mmol) and 4-bromo-1,1-difluorobutane (23.95 mg, 0.138 mmol) to give the title compound as a pale-yellow solid (10 mg, 26% yield). ¹H NMR (500 MHz, DMSO-d₆) δ=11.34-11.10 (m, 1H), 8.30-8.21 (m, 1H), 7.92-7.82 (m, 1H), 7.76-7.66 (m, 2H), 7.43 (br d, J=7.3 Hz, 3H), 7.02-6.91 (m, 2H), 6.32-6.24 (m, 2H), 6.24-5.97 (m, 1H), 3.17 (br s, 4H), 2.58-2.52 (m, 4H), 2.39-2.33 (m, 2H), 1.92-1.79 (m, 2H), 1.63-1.53 (m, 2H); LCMS: [M+H]⁺=526.50.

### Example 492: 6-(2-amino-5-(4-(4-cyclobutylpiperazin-1-yl)phenyl)-6-fluoropyridin-3-yl)-4-methylisoquinolin-1(2H)-one Example 366: one (I-545)

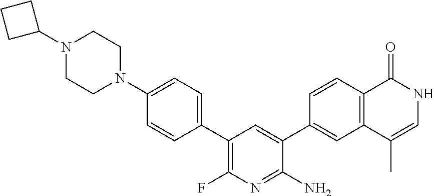 [SMILES: Cc1c[nH]c(=O)c2ccc(-c3cc(-c4ccc(N5CCN(C6CCC6)CC5)cc4)c(F)nc3N)cc12]

Prepared from 6-(2-amino-5-bromo-6-fluoropyridin-3-yl)-4-methylisoquinolin-1(2H)-one (35 mg, 0.101 mmol) and 1-cyclobutyl-4-(4-(4,4,5,5-tetramethyl-1,3,2-dioxaborolan-2-yl)phenyl)piperazine (41.3 mg, 0.121 mmol) to give the product (22.8 mg, 46.9% yield) as a white solid. ¹H NMR (500 MHz, DMSO-d₆) δ ppm 11.09 (d, J=5.01 Hz, 1 H), 8.27 (d, J=8.19 Hz, 1H), 7.72 (s, 1H), 7.60-7.69 (m, 2H), 7.41 (d, J=8.07 Hz, 2H), 7.04 (d, J=4.89 Hz, 1H), 6.97 (d, J=8.68 Hz, 2H), 6.24 (s, 2H), 3.15 (d, J=4.28 Hz, 4H), 2.73 (quin, J=7.64 Hz, 1H), 2.37 (d, J=4.52 Hz, 4H), 2.24 (s, 3H), 1.93-2.03 (m, 2H), 1.81 (quin, J=9.45 Hz, 2H), 1.59-1.71 (m, 2H); LCMS: [M+H]⁺=484.51.

### Example 493: 6-(2-amino-5-(4-(4-cyclobutylpiperazin-1-yl)phenyl)-6-fluoropyridin-3-yl)-4-fluoroisoquinolin-1(2H)-one (I-546)

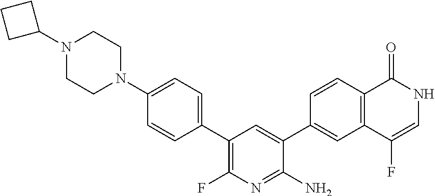 [SMILES: Nc1nc(F)c(-c2ccc(N3CCN(C4CCC4)CC3)cc2)cc1-c1ccc2c(=O)[nH]cc(F)c2c1]

Prepared from 6-(2-amino-5-bromo-6-fluoropyridin-3-yl)-4-fluoroisoquinolin-1(2H)-one (35 mg, 0.099 mmol) and 1-cyclobutyl-4-(4-(4,4,5,5-tetramethyl-1,3,2-dioxaborolan-2-yl)phenyl)piperazine (40.8 mg, 0.119 mmol) to give the title compound (16.7 mg, 34.5% yield) as a grey solid. ¹H NMR (500 MHz, DMSO-d₆) δ ppm 11.16 (br. s., 1H), 8.26 (dd, J=8.31, 1.71 Hz, 1H), 7.82 (d, J=0.98 Hz, 1H), 7.73 (dd, J=8.31, 1.35 Hz, 1H), 7.67 (d, J=10.15 Hz, 1H), 7.42 (d, J=7.83 Hz, 3H), 6.97 (d, J=8.80 Hz, 2H), 6.29 (s, 2H), 3.11-3.18 (m, 4H), 2.73 (quin, J=7.76 Hz, 1H), 2.35-2.41 (m, 4H), 1.95-2.02 (m, 2H), 1.77-1.86 (m, 2H), 1.60-1.70 (m, 2H); LCMS: [M+H]⁺=488.45.

### Example 494: 6-(2-amino-6-fluoro-5-(4-(4-isopropylpiperazin-1-yl)phenyl)pyridin-3-yl)-4,7-difluoroisoquinolin-1(2H)-one (I-547)

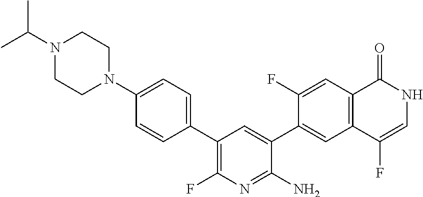 [SMILES: CC(C)N1CCN(c2ccc(-c3cc(-c4cc5c(F)c[nH]c(=O)c5cc4F)c(N)nc3F)cc2)CC1]

Step 1: 6-bromo-4,7-difluoroisoquinolin-1(2H)-one

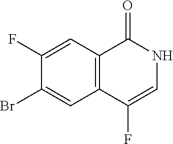 [SMILES: O=c1[nH]cc(F)c2cc(Br)c(F)cc12]

A RBF was charged with 6-bromo-7-fluoro-2H-isoquinolin-1-one (800 mg, 3.31 mmol) and Selectfluor™ fluorinating reagent (1405 mg, 3.97 mmol). Methanol (8 ml) and acetonitrile (8 ml) were added, and the reaction was heated to 50° C. and stirred for 1 h. The reaction was concentrated in vacuo, dissolved in 1,2-dichloroethane (16 mL), and phosphorous (V) oxychloride (0.618 ml, 6.61 mmol) was added. The reaction was stirred at 50° C. for 1 h then quenched into ice water and extracted with CH₂Cl₂ (2x). The combined organic layers were dried with anhydrous Na₂SO₄ and concentrated to afford the product (710 mg, 83%) as a white solid. The material was used in the next step without further purification. LCMS: [M−H]−=258.09.

Step 2: (4,7-difluoro-1-oxo-1,2-dihydroisoquinolin-6-yl)boronic acid

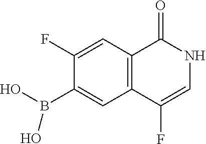 [SMILES: O=c1[nH]cc(F)c2cc(B(O)O)c(F)cc12]

Prepared from 6-bromo-4,7-difluoroisoquinolin-1(2H)-one (710 mg, 2.73 mmol) to give the boronic acid which was used in the next step without further purification. LCMS: [M+H]+ 226.12.

Step 3: 6-(2-amino-5-bromo-6-fluoropyridin-3-yl)-4,7-difluoroisoquinolin-1(2H)-one

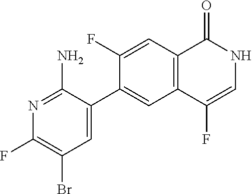 [SMILES: Nc1nc(F)c(Br)cc1-c1cc2c(F)c[nH]c(=O)c2cc1F]

Prepared from 5-bromo-6-fluoro-3-iodopyridin-2-amine (352 mg, 1.111 mmol) and (4,7-difluoro-1-oxo-1,2-dihydroisoquinolin-6-yl)boronic acid (300 mg, 1.333 mmol) to give the product (246 mg, 59.8% yield) as a beige solid. LCMS: [M+H]⁺=370.18.

Step 4: 6-(2-amino-6-fluoro-5-(4-(4-isopropylpiperazin-1-yl)phenyl)pyridin-3-yl)-4,7-difluoroisoquinolin-1(2H)-one

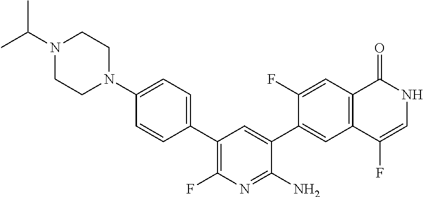 [SMILES: CC(C)N1CCN(c2ccc(-c3cc(-c4cc5c(F)c[nH]c(=O)c5cc4F)c(N)nc3F)cc2)CC1]

Prepared from 6-(2-amino-5-bromo-6-fluoropyridin-3-yl)-4,7-difluoroisoquinolin-1(2H)-one (35 mg, 0.095 mmol) and 4-(4-isopropylpiperazinyl)phenylboronic acid, pinacol ester (37.5 mg, 0.113 mmol) to give the title compound (17.2 mg, 36.9% yield) as a grey solid. 1H NMR (500 MHz, DMSO-d6) δ ppm 11.29 (br. s., 1H), 7.96 (d, J=9.29 Hz, 1H), 7.81 (d, J=6.72 Hz, 1H), 7.68 (d, J=10.15 Hz, 1 H), 7.36-7.44 (m, 3H), 6.96 (d, J=8.68 Hz, 2H), 6.28 (s, 2H), 3.12-3.16 (m, 4H), 2.64-2.70 (m, 1H), 2.56-2.59 (m, 4H), 1.00 (d, J=6.48 Hz, 6H); LCMS [M+H]+ 494.53.

### Example 495: 6-(2-amino-5-(4-(4-(cyclopropylmethyl)piperazin-1-yl)phenyl)-6-fluoropyridin-3-yl)-4,7-difluoroisoquinolin-1(2H)-one (I-548)

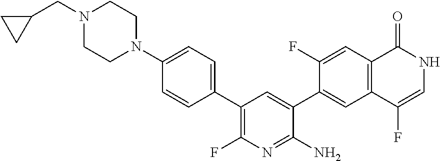 [SMILES: Nc1nc(F)c(-c2ccc(N3CCN(CC4CC4)CC3)cc2)cc1-c1cc2c(F)c[nH]c(=O)c2cc1F]

Prepared from 6-(2-amino-5-bromo-6-fluoropyridin-3-yl)-4,7-difluoroisoquinolin-1(2H)-one (35 mg, 0.095 mmol) and 1-(cyclopropylmethyl)-4-(4-(4,4,5,5-tetramethyl-1,3,2-dioxaborolan-2-yl)phenyl)piperazine (38.8 mg, 0.113 mmol) to give the title compound (8.2 mg, 17% yield) as a beige solid. ¹H NMR (500 MHz, DMSO-d₆) δ ppm 11.27 (br. s., 1H), 7.94-7.98 (m, 1H), 7.81 (d, J=6.85 Hz, 1H), 7.68 (d, J=10.15 Hz, 1H), 7.37-7.43 (m, 3H), 6.97 (d, J=8.93 Hz, 2H), 6.28 (s, 2H), 3.15-3.20 (m, 4H), 2.56-2.59 (m, 4 H), 2.22 (d, J=6.60 Hz, 2H), 0.81-0.91 (m, 1H), 0.45-0.51 (m, 2H), 0.07-0.12 (m, 2H); LCMS: [M+H]⁺=506.50.

### Example 496: 6-(2-amino-5-(3-(azetidin-1-ylmethyl)-4-morpholinophenyl)-6-fluoropyridin-3-yl)-3,4-dihydroisoquinolin-1(2H)-one (I-549)

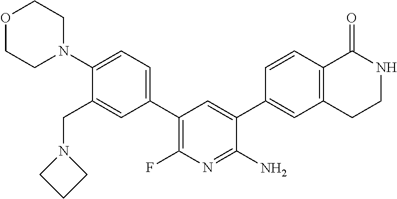 [SMILES: Nc1nc(F)c(-c2ccc(N3CCOCC3)c(CN3CCC3)c2)cc1-c1ccc2c(c1)CCNC2=O]

Prepared from 4-(2-(azetidin-1-ylmethyl)-4-bromophenyl)morpholine (50 mg, 0.153 mmol, in turn made from 5-bromo-2-morpholinobenzaldehyde and azetidine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (76 mg, 0.198 mmol) to yield the title compound as a white solid (67 mg, 86% yield). 1H NMR (500 MHz, DMSO-d₆) δ=8.00-7.93 (m, 1H), 7.93-7.87 (m, 1H), 7.61-7.55 (m, 1H), 7.50-7.43 (m, 3H), 7.41-7.34 (m, 1H), 7.13-7.07 (m, 1H), 6.26 (s, 2H), 3.79-3.72 (m, 4H), 3.63-3.57 (m, 2H), 3.44-3.39 (m, 2H), 3.20-3.13 (m, 4H), 2.98-2.94 (m, 2H), 2.94-2.89 (m, 4H), 2.04-1.91 (m, 2H); LCMS: [M+H]⁺=488.58.

### Example 497: 6-(2-amino-5-(3-(azetidin-1-ylmethyl)-4-morpholinophenyl)-6-fluoropyridin-3-yl)-7-fluoro-3,4-dihydroisoquinolin-1(2H)-one (I-550)

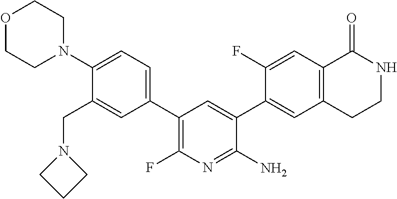 [SMILES: Nc1nc(F)c(-c2ccc(N3CCOCC3)c(CN3CCC3)c2)cc1-c1cc2c(cc1F)C(=O)NCC2]

Prepared from 4-(2-(azetidin-1-ylmethyl)-4-bromophenyl)morpholine (50 mg, 0.153 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (80 mg, 0.198 mmol) to yield the title compound as a white solid (42 mg, 52% yield). ¹H NMR (500 MHz, DMSO-d₆) δ=8.24-8.05 (m, 1H), 7.69-7.55 (m, 2H), 7.50-7.45 (m, 1H), 7.45-7.39 (m, 1H), 7.39-7.32 (m, 1H), 7.16-7.05 (m, 1H), 6.34-6.22 (m, 2H), 3.74 (br s, 4H), 3.59 (br s, 2H), 3.46-3.40 (m, 2H), 3.16 (br s, 4H), 2.92 (br s, 6H), 1.98 (br s, 2H); LCMS: [M+H]⁺=506.50

### Example 498: 6-(6′-amino-6-((dimethylamino)methyl)-2′-fluoro-5-morpholino-[2,3′-bipyridin]-5′-yl)-3,4-dihydroisoquinolin-1(2H)-one (I-551)

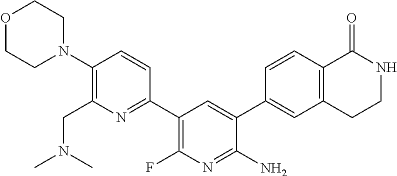 [SMILES: CN(C)Cc1nc(-c2cc(-c3ccc4c(c3)CCNC4=O)c(N)nc2F)ccc1N1CCOCC1]

Step 1: 6-bromo-3-morpholinopicolinaldehyde

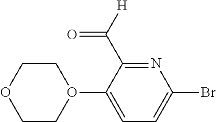

Prepared aby the reaction between 2-bromo-5-fluoro-6-formylpyridine (2.5 g, 12.25 mmol) and morpholine (1.269 ml, 14.71 mmol) at 85° C. for 6 h. The title compound was isolated as a pale-yellow oil (2.747 g, 79% yield). LCMS: [M+H]⁺=271.18

Step 2: 1-(6-bromo-3-morpholinopyridin-2-yl)-N,N-dimethylmethanamine

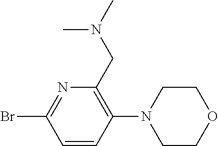 [SMILES: CN(C)Cc1nc(Br)ccc1N1CCOCC1]

Prepared using 6-bromo-3-morpholinopicolinaldehyde (500 mg, 1.752 mmol) and Dimethylamine, 2.0M solution in THF (0.876 ml, 1.752 mmol). Silica gel chromatography (eluting with CH2Cl2 containing 0-2% MeOH and 0-0.2% NH4OH) afforded the title product as a cream colored solid (336 mg, 62% yield). LCMS: [M+H]⁺=300.35

Step 3: 6-(6′-amino-6-((dimethylamino)methyl)-2′-fluoro-5-morpholino-[2,3′-bipyridin]-5′-yl)-3,4-dihydroisoquinolin-1(2H)-one

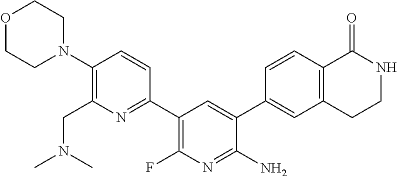 [SMILES: CN(C)Cc1nc(-c2cc(-c3ccc4c(c3)CCNC4=O)c(N)nc2F)ccc1N1CCOCC1]

Prepared from 1-(6-bromo-3-morpholinopyridin-2-yl)-N,N-dimethylmethanamine (50 mg, 0.162 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (81 mg, 0.211 mmol) to yield the title compound as a white solid (61 mg, 75% yield). ¹H NMR (500 MHz, DMSO-d₆) δ=8.12-8.03 (m, 1H), 7.99-7.94 (m, 1H), 7.94-7.89 (m, 1H), 7.68-7.60 (m, 1H), 7.58-7.51 (m, 1H), 7.45-7.37 (m, 2H), 6.55-6.34 (m, 2H), 3.81-3.72 (m, 4H), 3.56-3.51 (m, 2H), 3.44-3.40 (m, 2H), 3.11-3.01 (m, 4H), 2.96 (br t, J=6.4 Hz, 2H), 2.30-2.16 (m, 6H); LCMS: [M+H]⁺=477.49.

### Example 499: 6-(2-amino-5-(3-((ethyl(methyl)amino)methyl)-4-(tetrahydro-2H-pyran-4-yl)phenyl)-6-fluoropyridin-3-yl)-7-fluoro-3,4-dihydroisoquinolin-1(2H)-one (I-552)

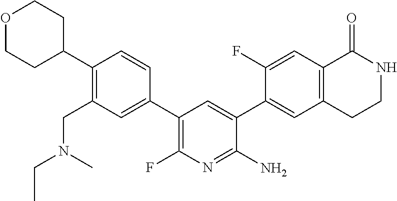 [SMILES: CCN(C)Cc1cc(-c2cc(-c3cc4c(cc3F)C(=O)NCC4)c(N)nc2F)ccc1C1CCOCC1]

Prepared from N-(5-bromo-2-(tetrahydro-2H-pyran-4-yl)benzyl)-N-methylethanamine (55 mg, 0.167 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (87 mg, 0.218 mmol) to yield the title compound as a white solid (47 mg, 53% yield). ¹H NMR (500 MHz, DMSO-d₆) δ=8.17-8.07 (m, 1H), 7.65-7.58 (m, 2H), 7.45-7.40 (m, 2H), 7.39-7.36 (m, 1H), 7.35-7.32 (m, 1H), 6.43-6.23 (m, 2H), 4.00-3.94 (m, 2H), 3.51-3.47 (m, 2H), 3.46-3.40 (m, 4H), 3.27-3.19 (m, 1H), 2.94-2.89 (m, 2H), 2.40 (q, J=7.1 Hz, 2H), 2.13-2.07 (m, 3H), 1.76-1.67 (m, 2H), 1.65-1.60 (m, 2H), 1.08-1.02 (m, 3H); LCMS: [M+H]⁺=507.51.

### Example 500: 6-(2-amino-6-fluoro-5-(3-(((2-methoxyethyl)(methyl)amino)methyl)-4-morpholinophenyl)pyridin-3-yl)-3,4-dihydroisoquinolin-1(2H)-one (I-553)

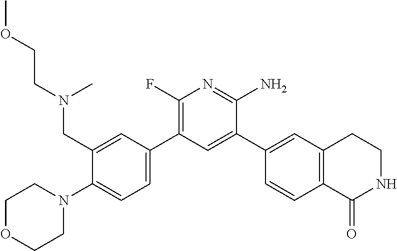 [SMILES: COCCN(C)Cc1cc(-c2cc(-c3ccc4c(c3)CCNC4=O)c(N)nc2F)ccc1N1CCOCC1]

Prepared from N-(5-bromo-2-morpholinobenzyl)-2-methoxy-N-methylethan-1-amine (60 mg, 0.175 mmol, in turn made from 5-bromo-2-morpholinobenzaldehyde and N-(2-methoxyethyl)methylamine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (87 mg, 0.227 mmol) to yield the title compound as a white solid (68 mg, 71% yield). ¹H NMR (500 MHz, DMSO-d₆) δ=7.98-7.93 (m, 1H), 7.93-7.89 (m, 1H), 7.61-7.56 (m, 2H), 7.49-7.39 (m, 3H), 7.18-7.10 (m, 1H), 6.30-6.19 (m, 2H), 3.77-3.71 (m, 4H), 3.60-3.53 (m, 2H), 3.45-3.42 (m, 2H), 3.28 (s, 2H), 3.19-3.14 (m, 3H), 2.98-2.94 (m, 2H), 2.94-2.89 (m, 4H), 2.56-2.52 (m, 2H), 2.25-2.19 (m, 3H); LCMS: [M+H]⁺=520.61.

### Example 501: 6-(2-amino-6-fluoro-5-(3-(((2-methoxyethyl)(methyl)amino)methyl)-4-morpholinophenyl)pyridin-3-yl)-7-fluoro-3,4-dihydroisoquinolin-1(2H)-one (I-554)

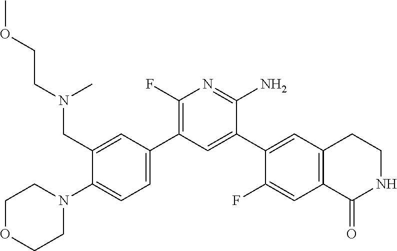 [SMILES: COCCN(C)Cc1cc(-c2cc(-c3cc4c(cc3F)C(=O)NCC4)c(N)nc2F)ccc1N1CCOCC1]

Prepared from N-(5-bromo-2-morpholinobenzyl)-2-methoxy-N-methylethan-1-amine (60 mg, 0.175 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (87 mg, 0.227 mmol) to yield the title compound as a white solid (68 mg, 71% yield). ¹H NMR (500 MHz, DMSO-d₆) b=7.98-7.93 (m, 1H), 7.93-7.89 (m, 1H), 7.61-7.56 (m, 2H), 7.49-7.39 (m, 3H), 7.18-7.10 (m, 1H), 6.30-6.19 (m, 2H), 3.77-3.71 (m, 4H), 3.60-3.53 (m, 2H), 3.45-3.42 (m, 2H), 3.28 (s, 2H), 3.19-3.14 (m, 3H), 2.98-2.94 (m, 2H), 2.94-2.89 (m, 4H), 2.56-2.52 (m, 2H), 2.25-2.19 (m, 3H); LCMS: [M+H]⁺=520.61.

### Example 502: 6-(2-amino-5-(4-(2,2-difluoroethoxy)-3-((dimethylamino)methyl)phenyl)-6-fluoropyridin-3-yl)-3,4-dihydroisoquinolin-1(2H)-one (I-555)

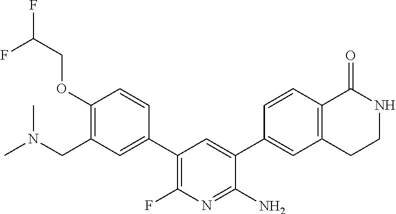 [SMILES: CN(C)Cc1cc(-c2cc(-c3ccc4c(c3)CCNC4=O)c(N)nc2F)ccc1OCC(F)F]

Prepared from 1-(5-bromo-2-(2,2-difluoroethoxy)phenyl)-N,N-dimethylmethanamine (55 mg, 0.178 mmol, in turn made from 5-bromo-2-(2,2-difluoroethoxy)benzaldehyde and dimethylamine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (88 mg, 0.231 mmol) to afford the title compound as an off white solid (69 mg, 78% yield). ¹H NMR (500 MHz, DMSO-d₆) δ=7.99-7.94 (m, 1H), 7.93-7.90 (m, 1H), 7.63-7.58 (m, 1H), 7.50-7.46 (m, 2H), 7.46-7.41 (m, 2H), 7.13-7.08 (m, 1H), 6.54-6.30 (m, 1H), 6.29-6.22 (m, 2H), 4.42-4.29 (m, 2H), 3.47-3.43 (m, 2H), 3.42-3.40 (m, 2H), 2.98-2.94 (m, 2H), 2.21-2.15 (m, 6H); LCMS: [M+H]⁺=471.53.

### Example 503: 6-(2-amino-5-(4-(2,2-difluoroethoxy)-3-((dimethylamino)methyl)phenyl)-6-fluoropyridin-3-yl)-7-fluoro-3,4-dihydroisoquinolin-1(2H)-one (I-556)

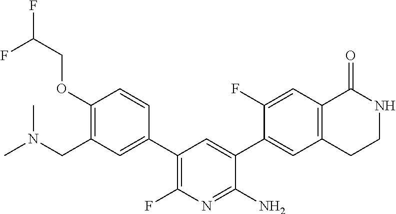 [SMILES: CN(C)Cc1cc(-c2cc(-c3cc4c(cc3F)C(=O)NCC4)c(N)nc2F)ccc1OCC(F)F]

Prepared from 1-(5-bromo-2-(2,2-difluoroethoxy)phenyl)-N,N-dimethylmethanamine (55 mg, 0.178 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (93 mg, 0.231 mmol) to afford the title compound as a white solid (45 mg, 49% yield). ¹H NMR (500 MHz, DMSO-d₆) δ=8.17-8.05 (m, 1H), 7.66-7.55 (m, 2H), 7.50-7.45 (m, 1H), 7.45-7.42 (m, 1H), 7.41 (br d, J=8.7 Hz, 1H), 7.16-7.03 (m, 1H), 6.52-6.29 (m, 1H), 6.26 (s, 1H), 4.39-4.28 (m, 2H), 3.46-3.43 (m, 2H), 3.42-3.39 (m, 2H), 2.96-2.89 (m, 2H), 2.23-2.11 (m, 6H); LCMS: [M+H]⁺=489.52.

### Example 504: 6-(2-amino-6-fluoro-5-(3-(pyrrolidin-1-ylmethyl)-4-(tetrahydro-2H-pyran-4-yl)phenyl)pyridin-3-yl)-3,4-dihydroisoquinolin-1(2H)-one (I-557)

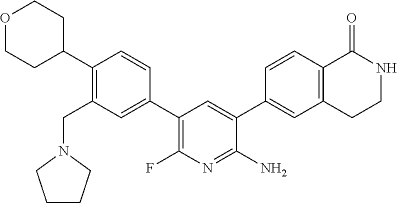 [SMILES: Nc1nc(F)c(-c2ccc(C3CCOCC3)c(CN3CCCC3)c2)cc1-c1ccc2c(c1)CCNC2=O]

Prepared from 1-(5-bromo-2-(tetrahydro-2H-pyran-4-yl)benzyl)pyrrolidine (60 mg, 0.148 mmol, in turn made from 5-bromo-2-(tetrahydro-2H-pyran-4-yl)benzaldehyde and pyrrolidine (0.275 ml, 3.34 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (73.7 mg, 0.192 mmol) to yield the title compound as an off white solid (67 mg, 86% yield). ¹H NMR (500 MHz, DMSO-d₆) δ=8.00-7.93 (m, 1H), 7.91 (br d, J=7.8 Hz, 1H), 7.67-7.60 (m, 1H), 7.50-7.40 (m, 4H), 7.33 (br d, J=7.8 Hz, 1H), 6.36-6.20 (m, 2H), 3.96 (br d, J=8.6 Hz, 2H), 3.69-3.64 (m, 2H), 3.48-3.40 (m, 4H), 3.21-3.15 (m, 1H), 2.99-2.92 (m, 2H), 2.45 (br s, 4H), 1.75-1.62 (m, 8H); LCMS: [M+H]⁺=501.62.

### Example 505: 6-(2-amino-6-fluoro-5-(3-(pyrrolidin-1-ylmethyl)-4-(tetrahydro-2H-pyran-4-yl)phenyl)pyridin-3-yl)-7-fluoro-3,4-dihydroisoquinolin-1(2H)-one (I-558)

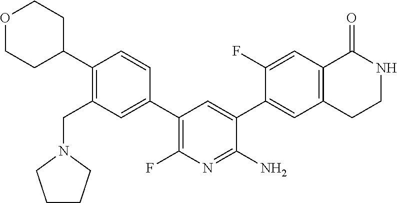 [SMILES: Nc1nc(F)c(-c2ccc(C3CCOCC3)c(CN3CCCC3)c2)cc1-c1cc2c(cc1F)C(=O)NCC2]

Prepared from 1-(5-bromo-2-(tetrahydro-2H-pyran-4-yl)benzyl)pyrrolidine (60 mg, 0.148 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (77 mg, 0.192 mmol) to yield the title compound as an off white solid (42 mg, 52% yield). ¹H NMR (500 MHz, DMSO-d₆) δ=8.11 (br s, 1H), 7.66-7.58 (m, 2H), 7.45-7.38 (m, 3H), 7.35-7.30 (m, 1H), 6.35-6.23 (m, 2H), 3.99-3.92 (m, 2H), 3.65 (s, 2H), 3.48-3.40 (m, 4H), 3.23-3.16 (m, 1H), 2.95-2.90 (m, 2H), 2.45 (br s, 4H), 1.72-1.60 (m, 8H); LCMS: [M+H]⁺=519.60.

### Example 506: 6-(2-amino-6-fluoro-5-(4-morpholino-3-(piperidin-1-ylmethyl)phenyl)pyridin-3-yl)-3,4-dihydroisoquinolin-1(2H)-one (I-559)

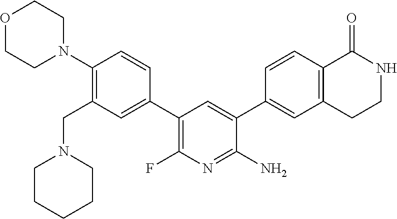 [SMILES: Nc1nc(F)c(-c2ccc(N3CCOCC3)c(CN3CCCCC3)c2)cc1-c1ccc2c(c1)CCNC2=O]

Prepared from 4-(4-bromo-2-(piperidin-1-ylmethyl)phenyl)morpholine (60 mg, 0.150 mmol, in turn made using 5-bromo-2-morpholinobenzaldehyde and piperidine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (74.9 mg, 0.195 mmol) to yield the title compound as a white solid (54 mg, 66% yield). ¹H NMR (500 MHz, DMSO-d₆) δ=7.98-7.93 (m, 1H), 7.93-7.89 (m, 1H), 7.63-7.57 (m, 1H), 7.55-7.50 (m, 1H), 7.49-7.46 (m, 1H), 7.45-7.43 (m, 1H), 7.43-7.38 (m, 1H), 7.18-7.11 (m, 1H), 6.30-6.21 (m, 2H), 3.78-3.71 (m, 4H), 3.51-3.46 (m, 2H), 3.45-3.39 (m, 2H), 3.01-2.92 (m, 6H), 2.45-2.33 (m, 4H), 1.51-1.43 (m, 4H), 1.42-1.33 (m, 2H); LCMS: [M+H]⁺=516.59.

### Example 507: 6-(2-amino-6-fluoro-5-(4-morpholino-3-(piperidin-1-ylmethyl)phenyl)pyridin-3-yl)-7-fluoro-3,4-dihydroisoquinolin-1(2H)-one (I-560)

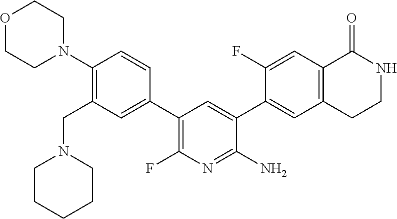 [SMILES: Nc1nc(F)c(-c2ccc(N3CCOCC3)c(CN3CCCCC3)c2)cc1-c1cc2c(cc1F)C(=O)NCC2]

Prepared from 4-(4-bromo-2-(piperidin-1-ylmethyl)phenyl)morpholine (60 mg, 0.150 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (78 mg, 0.195 mmol) to yield the title compound as a white solid (31 mg, 37% yield). ¹H NMR (500 MHz, DMSO-d₆) δ=8.17-8.04 (m, 1H), 7.66-7.55 (m, 2H), 7.53-7.48 (m, 1H), 7.46-7.41 (m, 1H), 7.41-7.36 (m, 1H), 7.18-7.10 (m, 1H), 6.45-6.09 (m, 2H), 3.82-3.71 (m, 4H), 3.48 (s, 2H), 3.44-3.39 (m, 2H), 2.99-2.87 (m, 6H), 2.43-2.27 (m, 4H), 1.55-1.43 (m, 4H), 1.42-1.35 (m, 2H); LCMS: [M+H]⁺=534.58.

### Example 508: 6-(2-amino-5-(3-((dimethylamino)methyl)-4-morpholinophenyl)-6-fluoropyridin-3-yl)-4,7-difluoroisoquinolin-1(2H)-one (I-561)

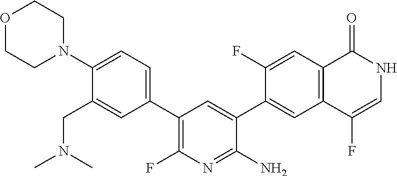 [SMILES: CN(C)Cc1cc(-c2cc(-c3cc4c(F)c[nH]c(=O)c4cc3F)c(N)nc2F)ccc1N1CCOCC1]

Prepared from 6-(2-amino-5-bromo-6-fluoropyridin-3-yl)-4,7-difluoroisoquinolin-1(2H)-one (35 mg, 0.095 mmol) and N,N-dimethyl-1-(2-morpholino-5-(4,4,5,5-tetramethyl-1,3,2-dioxaborolan-2-yl)phenyl)methanamine (39.3 mg, 0.113 mmol) to give the product (9.1 mg, 19% yield) as a grey solid. ¹H NMR (500 MHz, DMSO-d₆) δ ppm 11.29 (br. s., 1H), 7.97 (d, J=9.90 Hz, 1H), 7.82 (d, J=6.72 Hz, 1H), 7.69 (d, J=10.03 Hz, 1 H), 7.52 (br. s., 1H), 7.42 (d, J=5.26 Hz, 2H), 7.13 (d, J=8.19 Hz, 1H), 6.37 (br. s., 2H), 3.74 (br. s., 4H), 3.45 (br. s., 2H), 2.93 (br. s., 4H), 2.19 (br. s., 6H); LCMS: [M+H]⁺=510.58.

### Example 509 6-(2-amino-5-(3-((dimethylamino)methyl)-4-(tetrahydro-2H-pyran-4-yl)phenyl)-6-fluoropyridin-3-yl)-4,7-difluoroisoquinolin-1(2H)-one (I-562)

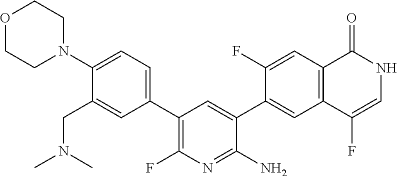 [SMILES: CN(C)Cc1cc(-c2cc(-c3cc4c(F)c[nH]c(=O)c4cc3F)c(N)nc2F)ccc1N1CCOCC1]

Prepared from 6-(2-amino-5-bromo-6-fluoropyridin-3-yl)-4,7-difluoroisoquinolin-1(2H)-one (35 mg, 0.095 mmol) and N,N-dimethyl-1-(2-(tetrahydro-2H-pyran-4-yl)-5-(4,4,5,5-tetramethyl-1,3,2-dioxaborolan-2-yl)phenyl)methanamine (39.2 mg, 0.113 mmol) to give the product (7.4 mg, 15% yield) as an off-white solid. ¹H NMR (500 MHz, DMSO-d₆) δ ppm 11.29 (d, J=5.50 Hz, 1H), 7.96 (d, J=10.03 Hz, 1H), 7.82 (d, J=6.72 Hz, 1 H), 7.73 (d, J=10.03 Hz, 1H), 7.37-7.46 (m, 3H), 7.34 (d, J=8.19 Hz, 1H), 6.39 (br. s., 2 H), 3.95 (dd, J=10.88, 3.18 Hz, 2H), 3.43 (d, J=10.39 Hz, 4H), 3.19 (t, J=11.68 Hz, 1H), 2.16 (br. s., 6H), 1.66-1.75 (m, 2H), 1.58-1.65 (m, 2H); LCMS: [M+H]⁺=509.57.

### Example 510 (R)-6-(2-amino-6-fluoro-5-(3-((3-methoxypyrrolidin-1-yl)methyl)-4-morpholinophenyl)pyridin-3-yl)-3,4-dihydroisoquinolin-1(2H)-one (I-563)

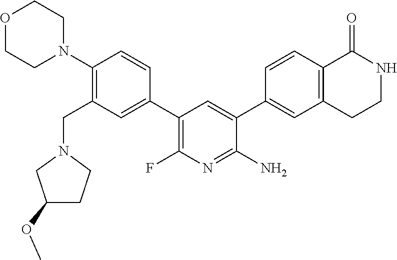 [SMILES: CO[C@@H]1CCN(Cc2cc(-c3cc(-c4ccc5c(c4)CCNC5=O)c(N)nc3F)ccc2N2CCOCC2)C1]

Prepared from (R)-4-(4-bromo-2-((3-methoxypyrrolidin-1-yl)methyl)phenyl)morpholine (60 mg, 0.160 mmol, in turn made from 5-bromo-2-morpholinobenzaldehyde and (R)-3-methoxy-pyrrolidine hydrochloride) and 6-(2-amino-6-fluoro-5-(4,4,5,5-tetramethyl-1,3,2-dioxaborolan-2-yl)pyridin-3-yl)-3,4-dihydroisoquinolin-1(2H)-one (80 mg, 0.209 mmol) to yield the title compound as a white solid (55 mg, 61% yield). ¹H NMR (500 MHz, DMSO-d₆) δ=7.99-7.93 (m, 1H), 7.91 (d, J=7.8 Hz, 1H), 7.63-7.57 (m, 1H), 7.55-7.49 (m, 1H), 7.47 (br d, J=7.9 Hz, 1H), 7.45 (s, 1H), 7.41 (br d, J=7.6 Hz, 1H), 7.18-7.07 (m, 1H), 6.44-6.18 (m, 2H), 3.92-3.85 (m, 1H), 3.79-3.73 (m, 4H), 3.64 (br s, 2H), 3.43-3.41 (m, 2H), 3.16-3.12 (m, 3H), 2.98-2.92 (m, 6H), 2.72-2.62 (m, 4H), 2.03-1.93 (m, 1H), 1.70-1.58 (m, 1H); LCMS: [M+H]⁺=532.64.

### Example 511 (R)-6-(2-amino-6-fluoro-5-(3-((3-methoxypyrrolidin-1-yl)methyl)-4-morpholinophenyl)pyridin-3-yl)-7-fluoro-3,4-dihydroisoquinolin-1(2H)-one (I-564)

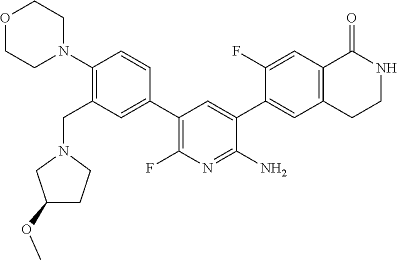 [SMILES: CO[C@@H]1CCN(Cc2cc(-c3cc(-c4cc5c(cc4F)C(=O)NCC5)c(N)nc3F)ccc2N2CCOCC2)C1]

Prepared from (R)-4-(4-bromo-2-((3-methoxypyrrolidin-1-yl)methyl)phenyl)morpholine (60 mg, 0.160 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (84 mg, 0.209 mmol) to yield the title compound as a white solid (40 mg, 43% yield). ¹H NMR (500 MHz, DMSO-d₆) δ=8.17-8.06 (m, 1H), 7.66-7.56 (m, 2H), 7.51 (br s, 1H), 7.46-7.42 (m, 1H), 7.41-7.33 (m, 1H), 7.19-7.05 (m, 1H), 6.36-6.14 (m, 2H), 3.91-3.84 (m, 1H), 3.75 (br s, 4H), 3.64 (br s, 2H), 3.44-3.40 (m, 2H), 3.14 (br s, 3H), 2.93 (br d, J=5.1 Hz, 6H), 2.74-2.58 (m, 4H), 2.04-1.94 (m, 1H), 1.73-1.59 (m, 1H); LCMS: [M+H]⁺=550.62.

### Example 512 (S)-6-(2-amino-6-fluoro-5-(3-((3-methoxypyrrolidin-1-yl)methyl)-4-morpholinophenyl)pyridin-3-yl)-3,4-dihydroisoquinolin-1(2H)-one (I-565)

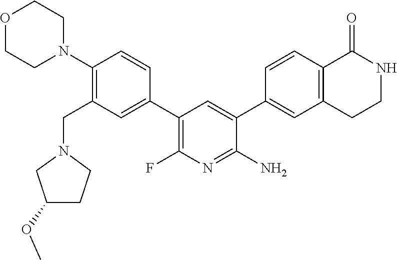 [SMILES: CO[C@H]1CCN(Cc2cc(-c3cc(-c4ccc5c(c4)CCNC5=O)c(N)nc3F)ccc2N2CCOCC2)C1]

Prepared from (S)-4-(4-bromo-2-((3-methoxypyrrolidin-1-yl)methyl)phenyl)morpholine (60 mg, 0.160 mmol, in turn made from 5-bromo-2-morpholinobenzaldehyde and (3S)-3-methoxypyrrolidine hydrochloride) and 6-(2-amino-6-fluoro-5-(4,4,5,5-tetramethyl-1,3,2-dioxaborolan-2-yl)pyridin-3-yl)-3,4-dihydroisoquinolin-1(2H)-one (80 mg, 0.209 mmol) to yield the title compound (62 mg, 69% yield). ¹H NMR (500 MHz, DMSO-d₆) δ 7.9-8.0 (m, 1H), 7.9-7.9 (m, 1H), 7.6-7.6 (m, 1H), 7.5-7.6 (m, 1H), 7.5-7.5 (m, 1H), 7.4-7.5 (m, 1H), 7.4-7.4 (m, 1H), 7.1-7.2 (m, 1H), 6.2-6.3 (m, 2H), 3.87 (br s, 1H), 3.75 (br s, 4H), 3.64 (br s, 2H), 3.42 (br s, 2H), 3.14 (br s, 3H), 2.9-3.0 (m, 6H), 2.6-2.8 (m, 4H), 1.9-2.1 (m, 1H), 1.5-1.8 (m, 1H); LCMS: [M+H]⁺=532.58.

### Example 513: (S)-6-(2-amino-6-fluoro-5-(3-((3-methoxypyrrolidin-1-yl)methyl)-4-morpholinophenyl)pyridin-3-yl)-7-fluoro-3,4-dihydroisoquinolin-1(2H)-one (I-566)

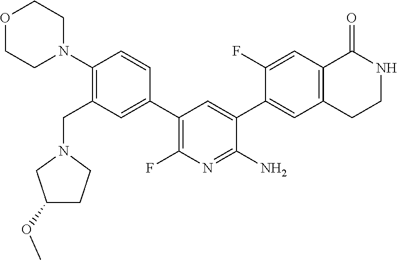 [SMILES: CO[C@H]1CCN(Cc2cc(-c3cc(-c4cc5c(cc4F)C(=O)NCC5)c(N)nc3F)ccc2N2CCOCC2)C1]

Prepared from (S)-4-(4-bromo-2-((3-methoxypyrrolidin-1-yl)methyl)phenyl)morpholine (60 mg, 0.160 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (84 mg, 0.209 mmol) to yield the title compound as a white solid (42 mg, 45% yield). ¹H NMR (500 MHz, DMSO-d₆) δ 8.1-8.2 (m, 1H), 7.6-7.6 (m, 2H), 7.5-7.6 (m, 1H), 7.4-7.4 (m, 1H), 7.39 (br d, 1H, J=5.7 Hz), 7.1-7.2 (m, 1H), 6.27 (br s, 2H), 3.8-3.9 (m, 1H), 3.75 (br s, 4H), 3.64 (br s, 2H), 3.41 (dt, 2H, J=2.7, 6.4 Hz), 3.14 (br s, 3H), 2.9-3.0 (m, 6H), 2.6-2.7 (m, 2H), 2.47 (br d, 2H, J=8.7 Hz), 1.97 (br d, 1H, J=6.2 Hz), 1.5-1.8 (m, 1H); LCMS: [M+H]⁺=550.62.

### Example 514: 6-(2-amino-5-(4-(4-(4,4-difluorobutyl)piperazin-1-yl)phenyl)-6-fluoropyridin-3-yl)-8-fluoro-3-methylisoquinolin-1(2H)-one (I-567)

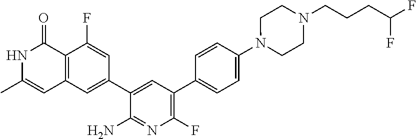 [SMILES: Cc1cc2cc(-c3cc(-c4ccc(N5CCN(CCCC(F)F)CC5)cc4)c(F)nc3N)cc(F)c2c(=O)[nH]1]

Step 1: 6-(2-amino-6-fluoro-5-(4-(piperazin-1-yl)phenyl)pyridin-3-yl)-8-fluoro-3-methylisoquinolin-1(2H)-one

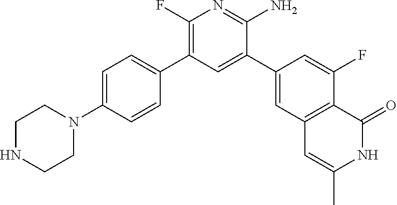 [SMILES: Cc1cc2cc(-c3cc(-c4ccc(N5CCNCC5)cc4)c(F)nc3N)cc(F)c2c(=O)[nH]1]

Prepared from 6-(2-amino-5-bromo-6-fluoropyridin-3-yl)-8-fluoro-3-methylisoquinolin-1(2H)-one (100 mg, 0.273 mmol), and 4-piperazinylphenylboronic acid, pinacol ester (102 mg, 0.355 mmol) as a beige solid (111 mg, 91%); LCMS: [M+H]+=448.47.

Step 2: 6-(2-amino-5-(4-(4-(4,4-difluorobutyl)piperazin-1-yl)phenyl)-6-fluoropyridin-3-yl)-8-fluoro-3-methylisoquinolin-1(2H)-one

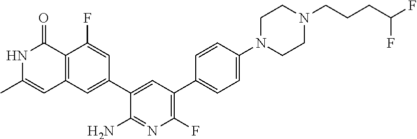 [SMILES: Cc1cc2cc(-c3cc(-c4ccc(N5CCN(CCCC(F)F)CC5)cc4)c(F)nc3N)cc(F)c2c(=O)[nH]1]

Prepared from 6-(2-amino-6-fluoro-5-(4-(piperazin-1-yl)phenyl)pyridin-3-yl)-8-fluoro-3-methylisoquinolin-1(2H)-one (50 mg, 0.112 mmol) and 4-bromo-1,1-difluorobutane (38.7 mg, 0.223 mmol) to give the title compound was isolated as an off white solid (38 mg, 60%). ¹H NMR (500 MHz, METHANOL-d₄) δ=7.72-7.66 (m, 1H), 7.51-7.47 (m, 1H), 7.45 (d, J=7.8 Hz, 2H), 7.28-7.20 (m, 1H), 7.08-7.02 (m, 2H), 6.52-6.45 (m, 1H), 6.09-5.81 (m, 1H), 3.29-3.24 (m, 4H), 2.70-2.64 (m, 4H), 2.52-2.46 (m, 2H), 2.36-2.29 (m, 3H), 1.96-1.83 (m, 2H), 1.77-1.68 (m, 2H); LCMS: [M+H]⁺=540.53.

### Example 515: 6-(5-(4-((1-acetylpiperidin-4-yl)oxy)-3-((dimethylamino)methyl)phenyl)-2-amino-6-fluoropyridin-3-yl)-3,4-dihydroisoquinolin-1(2H)-one (I-568)

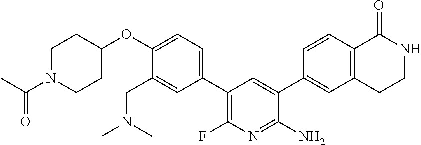 [SMILES: CC(=O)N1CCC(Oc2ccc(-c3cc(-c4ccc5c(c4)CCNC5=O)c(N)nc3F)cc2CN(C)C)CC1]

Step 1: tert-butyl 4-(4-bromo-2-formylphenoxy)piperidine-1-carboxylate

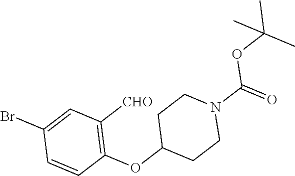 [SMILES: CC(C)(C)OC(=O)N1CCC(Oc2ccc(Br)cc2C=O)CC1]

To a mixture of 5-bromosalicylaldehyde (1 g, 4.97 mmol), KI (0.041 g, 0.249 mmol) and K₂CO₃ (2.063 g, 14.92 mmol) in DMF (10 mL) was added 1-Boc-4-(tosyloxy)piperidine (2.65 g, 7.46 mmol) and the reaction mixture was heated at 90° C. for 2.5 h. The reaction mixture was cooled to room temperature and partitioned between EtOAc and water. The organic layer was separated, washed with I N NaOH (15 mL), water (20 ml), dried over anhydrous Na₂SO₄, and concentrated to yield the product (1.625 g, 81% yield). LCMS: [M+H]⁺=328.24 (-tBu)

Step 2: tert-butyl 4-(4-bromo-2-((dimethylamino)methyl)phenoxy)piperidine-1-carboxylate

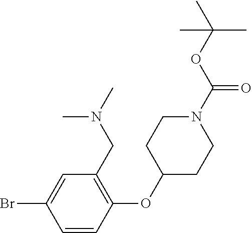 [SMILES: CN(C)Cc1cc(Br)ccc1OC1CCN(C(=O)OC(C)(C)C)CC1]

Prepared using tert-butyl 4-(4-bromo-2-formylphenoxy)piperidine-1-carboxylate (350 mg, 0.911 mmol) and dimethylamine, 2.0M solution in THF (1.822 mL, 3.64 mmol) to give the product as a pale-yellow oil (401 mg, quantitative yield). LCMS: [M+H]⁺=413.36

Step 3: tert-butyl 4-(4-(6-amino-2-fluoro-5-(1-oxo-1,2,3,4-tetrahydroisoquinolin-6-yl)pyridin-3-yl)-2-((dimethylamino)methyl)phenoxy)piperidine-1-carboxylate

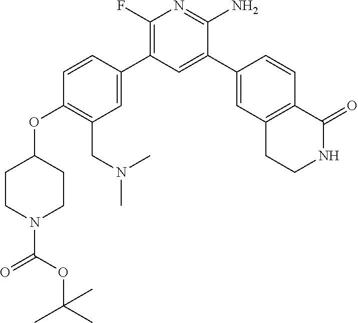 [SMILES: CN(C)Cc1cc(-c2cc(-c3ccc4c(c3)CCNC4=O)c(N)nc2F)ccc1OC1CCN(C(=O)OC(C)(C)C)CC1]

Prepared from tert-butyl 4-(4-bromo-2-((dimethylamino)methyl)phenoxy)piperidine-1-carboxylate (60 mg, 0.138 mmol) Example 146, step 2 with 6-(2-amino-6-fluoro-5-(4,4,5,5-tetramethyl-1,3,2-dioxaborolan-2-yl)pyridin-3-yl)-3,4-dihydroisoquinolin-1(2H)-one (68.7 mg, 0.179 mmol) to afford the title compound as a beige solid (60 mg, 61% yield). LCMS: [M+H]⁺=590.61.

Step 4: 6-(2-amino-5-(3-((dimethylamino)methyl)-4-(piperidin-4-yloxy)phenyl)-6-fluoropyridin-3-yl)-3,4-dihydroisoquinolin-1(2H)-one

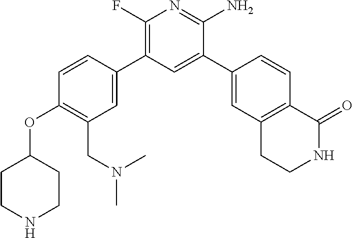 [SMILES: CN(C)Cc1cc(-c2cc(-c3ccc4c(c3)CCNC4=O)c(N)nc2F)ccc1OC1CCNCC1]

Prepared from tert-butyl 4-(4-(6-amino-2-fluoro-5-(1-oxo-1,2,3,4-tetrahydroisoquinolin-6-yl)pyridin-3-yl)-2-((dimethylamino)methyl)phenoxy)piperidine-1-carboxylate (60 mg, 0.084 mmol) and TFA (0.579 ml, 7.56 mmol) to give the product was isolated as a yellow solid (42 mg, 97%); LCMS: [M+H]⁺=490.58.

Step 5: 6-(5-(4-((1-acetylpiperidin-4-yl)oxy)-3-((dimethylamino)methyl)phenyl)-2-amino-6-fluoropyridin-3-yl)-3,4-dihydroisoquinolin-1(2H)-one

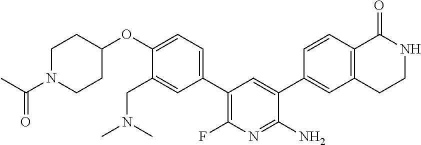 [SMILES: CC(=O)N1CCC(Oc2ccc(-c3cc(-c4ccc5c(c4)CCNC5=O)c(N)nc3F)cc2CN(C)C)CC1]

Prepared from 4 6-(2-amino-5-(3-((dimethylamino)methyl)-4-(piperidin-4-yloxy)phenyl)-6-fluoropyridin-3-yl)-3,4-dihydroisoquinolin-1(2H)-one (42 mg, 0.086 mmol) and Ac₂0 (8.11 μl, 0.086 mmol) to give the title compound as an off white solid (24 mg, 50%). ¹H NMR (500 MHz, METHANOL-d₄) δ=8.08-8.02 (m, 1H), 7.71-7.65 (m, 1H), 7.55-7.50 (m, 2H), 7.50-7.45 (m, 2H), 7.14-7.09 (m, 1H), 4.79-4.73 (m, 1H), 3.87-3.77 (m, 2H), 3.65-3.59 (m, 3H), 3.59-3.52 (m, 3H), 3.11-3.05 (m, 2H), 2.35-2.28 (m, 6H), 2.18-2.14 (m, 3H), 2.13-2.05 (m, 1H), 2.04-1.96 (m, 1H), 1.94-1.85 (m, 1H), 1.84-1.77 (m, 1H); LCMS: [M+H]⁺=532.58.

### Example 516: 6-(5-(4-((1-acetylpiperidin-4-yl)oxy)-3-((dimethylamino)methyl)phenyl)-2-amino-6-fluoropyridin-3-yl)-7-fluoro-3,4-dihydroisoquinolin-1(2H)-one (I-569)

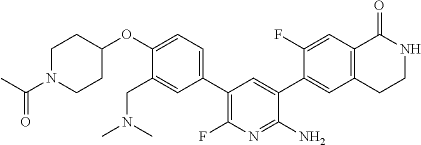 [SMILES: CC(=O)N1CCC(Oc2ccc(-c3cc(-c4cc5c(cc4F)C(=O)NCC5)c(N)nc3F)cc2CN(C)C)CC1]

Prepared from 2 6-(2-amino-5-(3-((dimethylamino)methyl)-4-(piperidin-4-yloxy)phenyl)-6-fluoropyridin-3-yl)-7-fluoro-3,4-dihydroisoquinolin-1(2H)-one (21 mg, 0.041 mmol) using a set of procedures analogous to the foregoing example to give the title compound as a white solid (15 mg, 63% yield). ¹H NMR (500 MHz, METHANOL-d₄) δ=7.79-7.73 (m, 1H), 7.70-7.65 (m, 1H), 7.52-7.49 (m, 1H), 7.48-7.44 (m, 1H), 7.44-7.40 (m, 1H), 7.14-7.10 (m, 1H), 4.78-4.73 (m, 1H), 3.87-3.77 (m, 2H), 3.66-3.60 (m, 3H), 3.58-3.53 (m, 3H), 3.07-3.01 (m, 2H), 2.34-2.29 (m, 6H), 2.17-2.14 (m, 3H), 2.12-2.06 (m, 1H), 2.05-1.98 (m, 1H), 1.93-1.86 (m, 1H), 1.84-1.78 (m, 1H); LCMS: [M+H]⁺=550.62.

### Example 517: 6-(2-amino-6-fluoro-5-(4-(4-isopropylpiperazin-1-yl)phenyl)pyridin-3-yl)-4-chloro-2,7-naphthyridin-1(2H)-one (I-570)

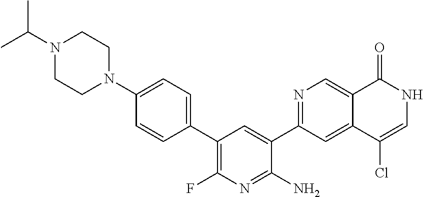 [SMILES: CC(C)N1CCN(c2ccc(-c3cc(-c4cc5c(Cl)c[nH]c(=O)c5cn4)c(N)nc3F)cc2)CC1]

Step 1: 6-bromo-4-chloro-2,7-naphthyridin-1(2H)-one

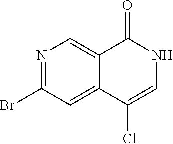 [SMILES: O=c1[nH]cc(Cl)c2cc(Br)ncc12]

To a solution of 6-bromo-2,7-naphthyridin-1(2H)-one (80 mg, 0.355 mmol) in N,N-dimethylacetamide (2 ml) was added N-chlorosuccinimide (57.0 mg, 0.427 mmol). The mixture was stirred at 50° C. for 1 hour. The reaction was cooled to RT and diluted with water. The white suspension was filtered, washed with water followed by methanol, and dried under vacuum to obtain 6-bromo-4-chloro-2,7-naphthyridin-1(2H)-one (61 mg, 66.1% yield) as a beige solid. LCMS: [M+H]⁺=259.22.

Step 2: 6-(2-amino-6-fluoro-5-(4-(4-isopropylpiperazin-1-yl)phenyl)pyridin-3-yl)-4-chloro-2,7-naphthyridin-1(2H)-one, hydrochloride

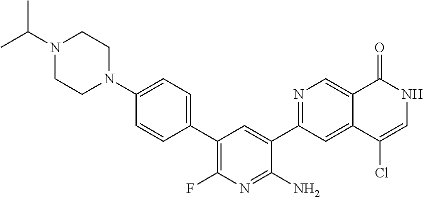 [SMILES: CC(C)N1CCN(c2ccc(-c3cc(-c4cc5c(Cl)c[nH]c(=O)c5cn4)c(N)nc3F)cc2)CC1]

Prepared from 6-bromo-4-chloro-2,7-naphthyridin-1(2H)-one (30 mg, 0.116 mmol) and 6-fluoro-5-(4-(4-isopropylpiperazin-1-yl)phenyl)-3-(4,4,5,5-tetramethyl-1,3,2-dioxaborolan-2-yl)pyridin-2-amine (61.1 mg, 0.139 mmol) to give the free base (4.3 mg) which was taken up in methanol (1 ml) and hydrochloric acid, 4.0 M in dioxane (0.032 ml, 0.127 mmol) to afford the title compound (3.1 mg, 7.5% overall yield) as a bright yellow solid. ¹H NMR (500 MHz, DMSO-d₆) δ ppm 11.93 (d, J=6.24 Hz, 1H), 10.34 (br. s., 1H), 9.40 (s, 1H), 8.27 (d, J=10.03 Hz, 1H), 8.02 (s, 1H), 7.77 (d, J=6.24 Hz, 1H), 7.52 (d, J=7.95 Hz, 2 H), 7.10 (d, J=8.80 Hz, 2H), 3.89-3.94 (m, 2H), 3.48-3.55 (m, 4H), 3.14-3.20 (m, 4H), 1.32 (d, J=6.60 Hz, 6H); LCMS: [M+H]⁺=493.47.

### Example 518: 6-(2-amino-6-fluoro-5-(4-(4-isopropylpiperazin-1-yl)phenyl)pyridin-3-yl)-8-fluoro-4-methylisoquinolin-1(2H)-one (I-571)

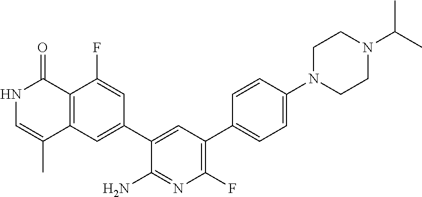 [SMILES: Cc1c[nH]c(=O)c2c(F)cc(-c3cc(-c4ccc(N5CCN(C(C)C)CC5)cc4)c(F)nc3N)cc12]

Step 1: (E)-3-(3-bromo-5-fluorophenyl)but-2-enoic acid

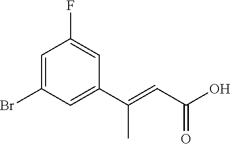

To THF (75 mL) at 0° C. under nitrogen atmosphere, sodium hydride (60%, 1.38 g, 34.56 mmol) was add portion wise. Triethyl phosphonoacetate (6.85 mL, 34.56 mmol) was added dropwise and stirred at 0° C. for 20 min. The resulting reaction mass was added dropwise to a solution of 1-(3-bromo-5-fluorophenyl)ethan-1-one (5 g, 23.04 mmol) in THF (50 mL) and the mixture was refluxed for 16 h. The mixture was then diluted with water (200 mL) and extracted with DCM (3×150 mL). The organic layer was dried over Na₂SO₄ and concentrated under vacuum to afford a yellow oil which was dissolved in MeOH (25 mL) and NaOH (4.6 g, 226.13 mmol) and water (15 mL) were added and the reaction was heated at 50° C. for 2 h. The mixture was concentrated and the resulting aqueous solution was acidified by aqueous 2M HCl and extracted with EtOAc (2×150 mL). The combined organic layers were dried over anhydrous Na₂SO₄ and concentrated under vacuum to afford the product (3.5 g, 58.6% yield) as white solid. LCMS: [M+H]⁺=258.9.

Step 2: (E)-3-(3-bromo-5-fluorophenyl)but-2-enoyl chloride

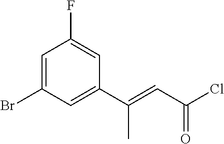

To a cooled solution of (E)-3-(3-bromo-5-fluorophenyl)but-2-enoic acid (3.5 g, 13.58 mmol) in DCM (35 mL) and DMF (0.2 mL) at 0° C., oxalyl chloride (2.05 g, 16.21 mmol) was added dropwise and the reaction was allowed to warm to RT and stirred for 3 h. The mixture was concentrated and azeotroped with toluene (2×20 mL) and DCM (2×20 mL) to give the crude product (3.5 g,) as white solid which was used directly in the next step without analysis.

Step 3: (E)-3-(3-bromo-5-fluorophenyl)but-2-enoyl azide

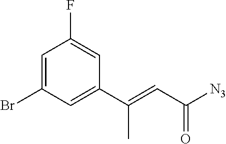 [SMILES: C/C(=C\C(=O)N=[N+]=[N-])c1cc(F)cc(Br)c1]

To a cooled solution of the (E)-3-(3-bromo-5-fluorophenyl)but-2-enoyl chloride (3.5 g, 12.61 mmol) in 1,4 dioxane (35 mL) at 0° C., a suspension of sodium azide (1.47 g, 22.7 mmol) in 1:1 mixture of 1,4-dioxane and water (15 mL) was added and the mixture was gradually warmed to room temperature and stirred for 1.5 h. The mixture was then diluted with water (30 mL) and extracted with diethyl ether (2×100 mL). The combined organic layer was back washed with saturated aqueous NaHCO₃ solution (3×100 mL) and water (3×100 mL) and dried over Na₂SO₄. The organic layer was directly used for next step.

Step 4: 6-bromo-8-fluoro-4-methylisoquinolin-1(2H)-one

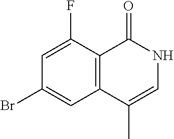

To the ether layer of (E)-3-(3-bromo-5-fluorophenyl)but-2-enoyl azide was treated with 1,2 dichlorobenzene (30 mL) and the ether was removed under vacuum to give a solution of (E)-3-(3-bromo-5-fluorophenyl)but-2-enoyl azide in 1,2 dichlorobenzene. The acyl azide solution in 1,2 dichlorobenzene was added dropwise over 30 min to a solution of iodine (0.4 g) in 1,2 dichlorobenzene (30 mL) at 120° C. The mixture was then stirred at 190° C. for 16 h, and allowed to cool room temperature and added to hexane (1000 mL). The suspension was stirred for 1 h and the resulting solid was filtered, washed with EtOAc (50 mL) and DCM (50 mL) and dried under vacuum to give the title compound (0.4 g, 12.7% yield) as pale yellow solid. LCMS: [M+H]⁺=258.

Step 5: (8-fluoro-4-methyl-1-oxo-1,2-dihydroisoquinolin-6-yl)boronic acid

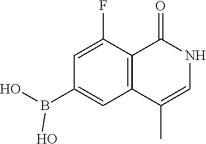 [SMILES: Cc1c[nH]c(=O)c2c(F)cc(B(O)O)cc12]

Prepared from 6-bromo-8-fluoro-4-methylisoquinolin-1(2H)-one (400 mg, 1.562 mmol) to give a mixture of the boronic acid and boronate which was used in the next step without further purification. LCMS: [M+H]+ 222.29 boronic acid, 304.42 boronate.

Step 6: 6-(2-amino-5-bromo-6-fluoropyridin-3-yl)-8-fluoro-4-methylisoquinolin-1(2H)-one

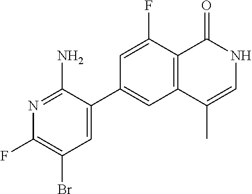 [SMILES: Cc1c[nH]c(=O)c2c(F)cc(-c3cc(Br)c(F)nc3N)cc12]

Prepared from 5-bromo-6-fluoro-3-iodopyridin-2-amine (200 mg, 0.631 mmol) and (8-fluoro-4-methyl-1-oxo-1,2-dihydroisoquinolin-6-yl)boronic acid (167 mg, 0.757 mmol) to give the product (90 mg, 38.9% yield) as a beige solid. LCMS: [M+H]⁺=366.29.

Step 7: 6-(2-amino-6-fluoro-5-(4-(4-isopropylpiperazin-1-yl)phenyl)pyridin-3-yl)-8-fluoro-4-methylisoquinolin-1(2H)-one

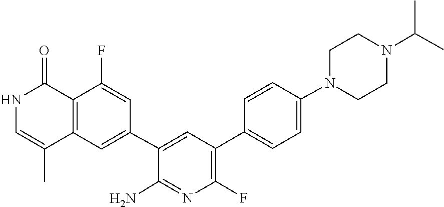 [SMILES: Cc1c[nH]c(=O)c2c(F)cc(-c3cc(-c4ccc(N5CCN(C(C)C)CC5)cc4)c(F)nc3N)cc12]

Prepared from 6-(2-amino-5-bromo-6-fluoropyridin-3-yl)-8-fluoro-4-methylisoquinolin-1(2H)-one (30 mg, 0.082 mmol) and 4-(4-isopropylpiperazinyl)phenylboronic acid, pinacol ester (32.5 mg, 0.098 mmol) to give the title compound (15.4 mg, 38.4% yield) as a beige solid. ¹H NMR (500 MHz, DMSO-d₆) δ ppm 11.08 (br. s., 1H), 7.69 (d, J=10.15 Hz, 1H), 7.50 (s, 1H), 7.41 (d, J=7.95 Hz, 2H), 7.36 (d, J=12.35 Hz, 1H), 7.06 (s, 1H), 6.97 (d, J=8.80 Hz, 2H), 6.33 (s, 2H), 3.10-3.18 (m, 4H), 2.63-2.70 (m, 1H), 2.55-2.60 (m, 4H), 2.21 (s, 3H), 1.00 (d, J=6.60 Hz, 6H); LCMS: [M +H]⁺=490.46.

### Example 519: 6-(2-amino-6-fluoro-5-(4-(4-isopropylpiperazin-1-yl)phenyl)pyridin-3-yl)-4,7-difluoro-3-methylisoquinolin-1(2H)-one (I-572)

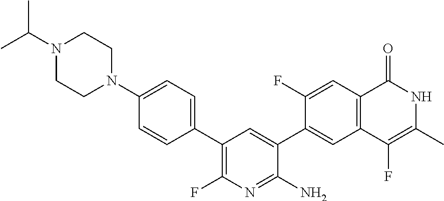 [SMILES: Cc1[nH]c(=O)c2cc(F)c(-c3cc(-c4ccc(N5CCN(C(C)C)CC5)cc4)c(F)nc3N)cc2c1F]

Step 1: 6-bromo-7-fluoro-3-methylisoquinolin-1(2H)-one

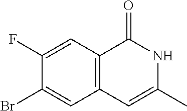

To a RBF was added copper(I) bromide (0.068 g, 0.472 mmol), Cs₂CO₃ (3.07 g, 9.43 mmol), 2,4-dibromo-5-fluorobenzamide (1.40 g, 4.72 mmol), propan-2-one (1.731 mL, 23.58 mmol) and dimethylsulfoxide (20 ml). The reaction was stirred and heated at 80° C. overnight. The reaction mixture was partitioned between brine (200 ml) and DCM (200 ml). The organic layer was separated, and the aqueous layer washed with DCM (2×100 mL). The combined organic layers were washed with brine, dried over anhydrous Na₂SO₄, and concentrated onto celite. The mixture was purified by flash chromatography eluting with 0-10% MeOH/DCM+1% NH₄OH. The desired fractions were collected, concentrated and dried under vacuum to afford the product (489 mg, 40.5% yield) as a beige solid. LCMS: [M+H]⁺=256.21.

Step 2: 6-bromo-4,7-difluoro-3-methylisoquinolin-1(2H)-one

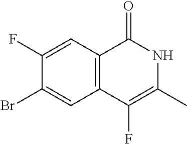

A vial was charged with 6-bromo-7-fluoro-3-methylisoquinolin-1(2H)-one (189 mg, 0.738 mmol) and Selectfluor™ fluorinating reagent (275 mg, 0.775 mmol). Methanol (10 ml) and acetonitrile (10 ml) were added, and the reaction was stirred at room temperature for 5 days. The white suspension was filtered, washed with water, and dried under vacuum to afford the product (158 mg, 78% yield) as a white solid. The material was used in the next step without further purification. LCMS [M+H]⁺=274.27.

Step 3: (4,7-difluoro-3-methyl-1-oxo-1,2-dihydroisoquinolin-6-yl)boronic acid

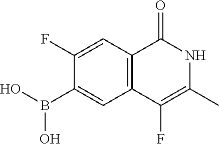 [SMILES: Cc1[nH]c(=O)c2cc(F)c(B(O)O)cc2c1F]

Prepared from 6-bromo-4,7-difluoro-3-methylisoquinolin-1(2H)-one (115 mg, 0.420 mmol) to give the boronic acid which was used in the next step without further purification. LCMS: [M+H]⁺=240.28.

Step 4: 6-(2-amino-6-fluoro-5-(4-(4-isopropylpiperazin-1-yl)phenyl)pyridin-3-yl)-4,7-difluoro-3-methylisoquinolin-1(2H)-one

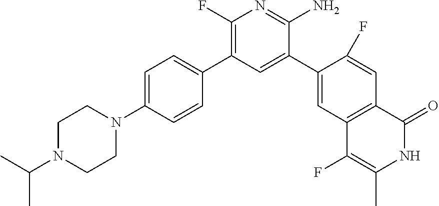 [SMILES: Cc1[nH]c(=O)c2cc(F)c(-c3cc(-c4ccc(N5CCN(C(C)C)CC5)cc4)c(F)nc3N)cc2c1F]

Prepared from (4,7-difluoro-3-methyl-1-oxo-1,2-dihydroisoquinolin-6-yl)boronic acid (32.8 mg, 0.137 mmol) and 3-bromo-6-fluoro-5-(4-(4-isopropylpiperazin-1-yl)phenyl)pyridin-2-amine (45 mg, 0.114 mmol) to give the title compound (6.2 mg, 10.7% yield) as a beige solid. ¹H NMR (500 MHz, DMSO-d₆) δ ppm 11.36 (br. s., 1H), 7.90 (d, J=9.90 Hz, 1H), 7.73 (d, J=6.72 Hz, 1H), 7.66 (d, J=10.15 Hz, 1H), 7.39 (d, J=8.07 Hz, 2 H), 6.96 (d, J=8.93 Hz, 2H), 6.26 (s, 2H), 3.12-3.17 (m, 4H), 2.64-2.70 (m, 1H), 2.56-2.58 (m, 4H), 2.23 (d, J=2.81 Hz, 3H), 1.00 (d, J=6.48 Hz, 6H); LCMS: [M+H]⁺=508.57.

### Example 520: 6-(2-amino-6-fluoro-5-(3-(((2-methoxyethyl)(methyl)amino)methyl)-4-(tetrahydro-2H-pyran-4-yl)phenyl)pyridin-3-yl)-3,4-dihydroisoquinolin-1(2H)-one (I-573)

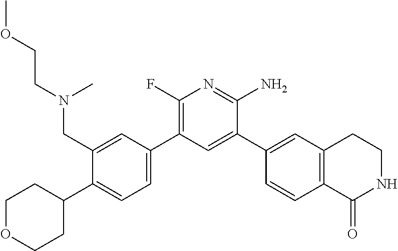 [SMILES: COCCN(C)Cc1cc(-c2cc(-c3ccc4c(c3)CCNC4=O)c(N)nc2F)ccc1C1CCOCC1]

Prepared from N-(5-bromo-2-(tetrahydro-2H-pyran-4-yl)benzyl)-2-methoxy-N-methylethan-1-amine (50 mg, 0.137 mmol, in turn made from bromo-2-(tetrahydro-2H-pyran-4-yl)benzaldehyde and N-(2-methoxyethyl)methylamine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (68.2 mg, 0.178 mmol) to yield the title compound as an off white solid (47 mg, 63% yield). ¹H NMR (500 MHz, DMSO-d₆) δ 7.9-8.0 (m, 1H), 7.91 (d, 1H, J=7.8 Hz), 7.6-7.7 (m, 1H), 7.5-7.5 (m, 1H), 7.4-7.5 (m, 2H), 7.4-7.4 (m, 1H), 7.3-7.4 (m, 1H), 6.1-6.4 (m, 2H), 3.9-4.0 (m, 2H), 3.5-3.6 (m, 2H), 3.4-3.5 (m, 4H), 3.4-3.4 (m, 2H), 3.2-3.3 (m, 1H), 3.2-3.2 (m, 3H), 2.9-3.0 (m, 2H), 2.5-2.6 (m, 2H), 2.1-2.2 (m, 3H), 1.7-1.8 (m, 2H), 1.6-1.7 (m, 2H); LCMS: [M+H]⁺=519.54

### Example 521: 6-(2-amino-6-fluoro-5-(3-(((2-methoxyethyl)(methyl)amino)methyl)-4-(tetrahydro-2H-pyran-4-yl)phenyl)pyridin-3-yl)-7-fluoro-3,4-dihydroisoquinolin-1(2H)-one (I-574)

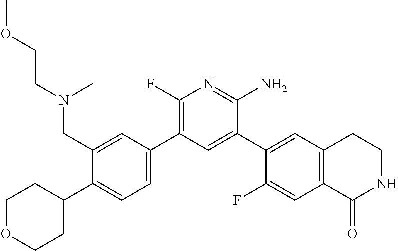 [SMILES: COCCN(C)Cc1cc(-c2cc(-c3cc4c(cc3F)C(=O)NCC4)c(N)nc2F)ccc1C1CCOCC1]

Prepared from N-(5-bromo-2-(tetrahydro-2H-pyran-4-yl)benzyl)-2-methoxy-N-methylethan-1-amine (55 mg, 0.151 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (79 mg, 0.196 mmol) to yield the title compound as a white solid (37 mg, 44% yield). ¹H NMR (500 MHz, DMSO-d₆) δ 8.1-8.1 (m, 1H), 7.6-7.7 (m, 2H), 7.4-7.4 (m, 2H), 7.4-7.4 (m, 1H), 7.3-7.4 (m, 1H), 6.2-6.3 (m, 2H), 3.9-4.0 (m, 2H), 3.5-3.6 (m, 2H), 3.4-3.5 (m, 4H), 3.4-3.4 (m, 2H), 3.2-3.3 (m, 1H), 3.2-3.2 (m, 3H), 2.92 (br t, 2H, J=6.4 Hz), 2.5-2.6 (m, 2H), 2.1-2.2 (m, 3H), 1.7-1.7 (m, 2H), 1.6-1.7 (m, 2H); LCMS: [M+H]⁺=537.59.

### Example 522: (R)-6-(2-amino-5-(3-((dimethylamino)methyl)-4-(2-methylmorpholino)phenyl)-6-fluoropyridin-3-yl)-3,4-dihydroisoquinolin-1(2H)-one (I-575)

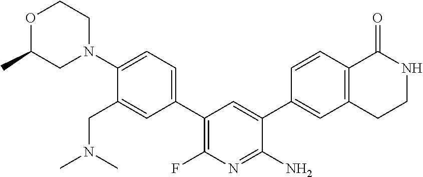 [SMILES: C[C@@H]1CN(c2ccc(-c3cc(-c4ccc5c(c4)CCNC5=O)c(N)nc3F)cc2CN(C)C)CCO1]

Step 1: (R)-5-bromo-2-(2-methylmorpholino)benzaldehyde

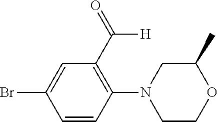 [SMILES: C[C@@H]1CN(c2ccc(Br)cc2C=O)CCO1]

A vial containing 5-bromo-2-fluorobenzaldehyde (5 g, 24.63 mmol), (R)-2-methyl-morpholine hydrochloride (3.39 g, 24.63 mmol) and K₂CO₃ (8.51 g, 61.6 mmol) in dry DMF (25 ml) was heated for 8 h in a in an oil bath at 120° C. The reaction mixture was partitioned between 450 ml water and 100 mL EtOAc, the organic phase was separated, aqueous phase was extracted with EtOAc (3×100 mL), the combined org phase was washed with water (1×250 ml), brine (1×250 ml), dried over Na₂SO₄ and concentrated. The crude product was purified by silica gel chromatography (eluting with hexanes containing 0-40% EtOAc) to afford the title compound as a pale-yellow oil (5.517 g, 79%). LCMS: [M+H]⁺=284.36.

Step 2: (R)-1-(5-bromo-2-(2-methylmorpholino)phenyl)-N,N-dimethylmethanamine

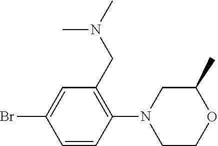 [SMILES: C[C@@H]1CN(c2ccc(Br)cc2CN(C)C)CCO1]

Prepared from (R)-5-bromo-2-(2-methylmorpholino)benzaldehyde (1 g, 3.52 mmol) and dimethylamine, 2.0M solution in THF (5.28 ml, 10.56 mmol) to give product as a colorless oil (1.118 g, 96% yield). LCMS: [M+H]⁺=313.32.

Step 3: (R)-6-(2-amino-5-(3-((dimethylamino)methyl)-4-(2-methylmorpholino)phenyl)-6-fluoropyridin-3-yl)-3,4-dihydroisoquinolin-1(2H)-one

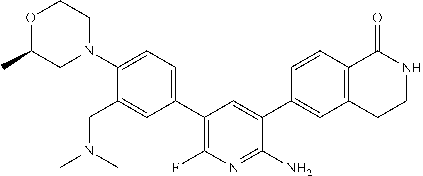 [SMILES: C[C@@H]1CN(c2ccc(-c3cc(-c4ccc5c(c4)CCNC5=O)c(N)nc3F)cc2CN(C)C)CCO1]

Prepared from (R)-1-(5-bromo-2-(2-methylmorpholino)phenyl)-N,N-dimethylmethanamine (50 mg, 0.160 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (80 mg, 0.208 mmol) to yield the title compound as a white solid (60 mg, 73% yield). ¹H NMR (500 MHz, DMSO-d₆) δ 7.94 (br s, 1H), 7.9-7.9 (m, 1H), 7.6-7.6 (m, 1H), 7.5-7.5 (m, 1H), 7.5-7.5 (m, 1H), 7.4-7.5 (m, 1H), 7.41 (br d, 1H, J=8.4 Hz), 7.1-7.1 (m, 1H), 6.2-6.3 (m, 2H), 3.8-3.9 (m, 1H), 3.7-3.8 (m, 2H), 3.4-3.5 (m, 2H), 3.4-3.4 (m, 2H), 3.1-3.2 (m, 1H), 3.0-3.1 (m, 1H), 2.9-3.0 (m, 2H), 2.7-2.8 (m, 1H), 2.4-2.5 (m, 1H), 2.2-2.2 (m, 6H), 1.1-1.2 (m, 3H); LCMS: [M+H]⁺=490.52.

### Example 523: (R)-6-(2-amino-5-(3-((dimethylamino)methyl)-4-(2-methylmorpholino)phenyl)-6-fluoropyridin-3-yl)-7-fluoro-3,4-dihydroisoquinolin-1(2H)-one (I-576)

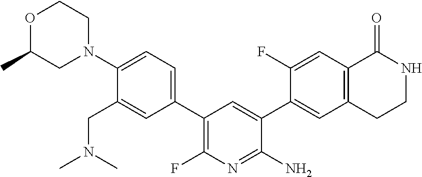 [SMILES: C[C@@H]1CN(c2ccc(-c3cc(-c4cc5c(cc4F)C(=O)NCC5)c(N)nc3F)cc2CN(C)C)CCO1]

Prepared in an analogous manner as the previous example from (R)-1-(5-bromo-2-(2-methylmorpholino)phenyl)-N,N-dimethylmethanamine (50 mg, 0.160 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (83 mg, 0.208 mmol) to yield the title compound as a white solid (24 mg, 28% yield). ¹H NMR (500 MHz, DMSO-d₆) δ 8.1-8.2 (m, 1H), 7.5-7.6 (m, 2H), 7.5-7.5 (m, 1H), 7.4-7.5 (m, 1H), 7.4-7.4 (m, 1H), 7.1-7.1 (m, 1H), 6.2-6.3 (m, 2H), 3.8-3.9 (m, 1H), 3.7-3.8 (m, 2H), 3.4-3.5 (m, 2H), 3.4-3.4 (m, 2H), 3.1-3.2 (m, 1H), 3.0-3.1 (m, 1H), 2.9-3.0 (m, 2H), 2.7-2.8 (m, 1H), 2.4-2.5 (m, 1H), 2.1-2.2 (m, 6H), 1.1-1.2 (m, 3H); LCMS: [M+H]⁺=508.57.

### Example 524: 6-(2-amino-6-fluoro-5-(4-(4-isopropylpiperazin-1-yl)phenyl)pyridin-3-yl)-7-fluoro-4-methylisoquinolin-1(2H)-one (I-577)

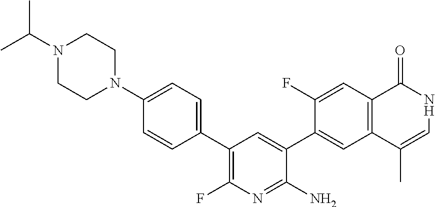 [SMILES: Cc1c[nH]c(=O)c2cc(F)c(-c3cc(-c4ccc(N5CCN(C(C)C)CC5)cc4)c(F)nc3N)cc12]

Step 1: (E)-3-(3-bromo-4-fluorophenyl)but-2-enoic acid

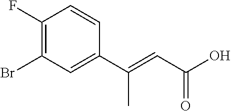

To THF (75 mL) at 0° C. under nitrogen atmosphere, sodium hydride (60%, 1.38 g, 34.56 mmol) was added portion wise. Triethyl phosphonoacetate (6.85 mL, 34.56 mmol) was added dropwise and stirred at 0° C. for 20 min and the resulting mixture was added dropwise to a solution of 1-(3-bromo-4-fluorophenyl)ethan-1-one (5 g, 23.04 mmol) in THF (50 mL) and the resulting mixture was refluxed for 16 h. The mixture was then diluted with water (200 mL) and extracted with DCM (3×150 mL). The organic layer was dried over Na₂SO₄ and concentrated undervacuum to afford a yellow oil. The oil was dissolved in MeOH (25 mL) and NaH (4.6 g, 226.13 mmol) and water (15 mL) were added and the reaction mass was heated at 50° C. for 2 h. The reaction mass was concentrated to evaporate the organics and the resulting aqueous solution was acidified by aqueous 2M HCl and extracted with EtOAc (2×150 mL). The combined organic layers were dried over Na₂SO₄ and concentrated under vacuum to afford the product (3.5 g, 58.6% yield) as white solid. LCMS: [M+H]⁺=258.9.

Step 2: (E)-3-(3-bromo-4-fluorophenyl)but-2-enoyl chloride

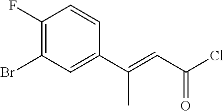

To a cooled solution of (E)-3-(3-bromo-4-fluorophenyl)but-2-enoic acid (3.5 g, 13.58 mmol) in DCM (35 mL) and DMF (0.2 mL) at 0° C., oxalyl chloride (2.05 g, 16.21 mmol) was added dropwise and the reaction mass was allowed to warm to RT and stirred at same temperature for 3 h. After completion the reaction, reaction mass was concentrated and azeotrope with toluene (2×20 mL) and DCM (2×20 mL) to give the crude product (3.5 g) as white solid which was used as such in the next step without analysis and further purification.

Step 3: (E)-3-(3-bromo-4-fluorophenyl)but-2-enoyl azide

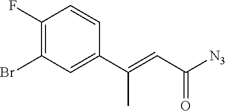 [SMILES: C/C(=C\C(=O)N=[N+]=[N-])c1ccc(F)c(Br)c1]

To a cooled solution of crude of (E)-3-(3-bromo-4-fluorophenyl)but-2-enoyl chloride (3.5 g, 12.61 mmol) in 1,4 dioxane (35 mL) at 0° C., a suspension of sodium azide (1.47 g, 22.70 mmol) in 1:1 mixture of 1,4-dioxane and water (15 mL) was added and the reaction mas was gradually warm to room temperature and stirred at same temperature for a 1.5 h. After completion reaction, reaction mass was diluted with water (30 mL) and extracted with diethyl ether (2×100 mL). The combined organic layers were back washed with saturated aq. NaHCO₃ (3×100 mL) and water (3×100 mL) and dried over Na₂SO₄ and organic layer was directly used for next step.

Step 4: 6-bromo-7-fluoro-4-methylisoquinolin-1(2H)-one

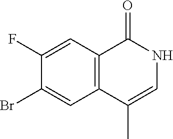

The ether layer of (E)-3-(3-bromo-4-fluorophenyl)but-2-enoyl azide was treated with 1,2 dichlorobenzene (30 mL) and the ether was removed under vacuum to give a solution of (E)-3-(3-bromo-4-fluorophenyl)but-2-enoyl azide in 1,2 dichlorobenzene. The acyl azide solution in 1,2 dichlorobenzene was added dropwise over 30 min to a solution of iodine (0.4 g) in 1,2 dichlorobenzene (30 mL) at 120° C. The mixture was then stirred at 190° C. for 16 h, allowed to cool room temperature and added to hexane (1000 mL). The suspension was stirred for 1 h and the resulting solid was washed with EtOAc (50 mL) and DCM (50 mL) and dried under vacuum to give the product (0.8 g, 25.36%) as pale-yellow solid. LCMS: [M+2]⁺=258.

Step 5: (7-fluoro-4-methyl-1-oxo-1,2-dihydroisoquinolin-6-yl)boronic acid

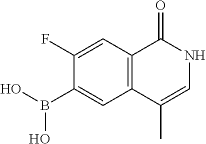 [SMILES: Cc1c[nH]c(=O)c2cc(F)c(B(O)O)cc12]

Prepared from 6-bromo-7-fluoro-4-methylisoquinolin-1(2H)-one (800 mg, 3.12 mmol) in a manner analogous to previous examples to give the boronic acid which was used in the next step without further purification. LCMS [M+H]⁺=222.29.

Step 6: 6-(2-amino-5-bromo-6-fluoropyridin-3-yl)-7-fluoro-4-methylisoquinolin-1(2H)-one

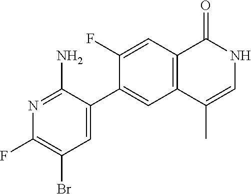 [SMILES: Cc1c[nH]c(=O)c2cc(F)c(-c3cc(Br)c(F)nc3N)cc12]

Prepared from 5-bromo-6-fluoro-3-iodopyridin-2-amine (400 mg, 1.262 mmol) and (7-fluoro-4-methyl-1-oxo-1,2-dihydroisoquinolin-6-yl)boronic acid (335 mg, 1.515 mmol) to give the product (357 mg, 77% yield) as a beige solid. LCMS [M+H]⁺=366.36.

Step 7: 6-(2-amino-6-fluoro-5-(4-(4-isopropylpiperazin-1-yl)phenyl)pyridin-3-yl)-7-fluoro-4-methylisoquinolin-1(2H)-one

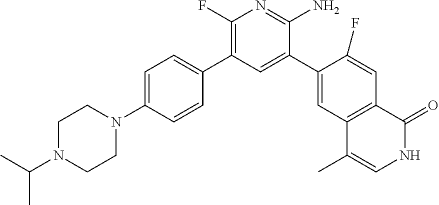 [SMILES: Cc1c[nH]c(=O)c2cc(F)c(-c3cc(-c4ccc(N5CCN(C(C)C)CC5)cc4)c(F)nc3N)cc12]

Prepared from 6-(2-amino-5-bromo-6-fluoropyridin-3-yl)-7-fluoro-4-methylisoquinolin-1(2H)-one (40 mg, 0.109 mmol) and 4-(4-isopropylpiperazinyl)phenylboronic acid, pinacol ester (43.3 mg, 0.131 mmol) to give the title compound (20.2 mg, 37.8% yield) as a grey solid. ¹H NMR (500 MHz, DMSO-d₆) 0 ppm 11.21 (br. s., 1H), 7.94 (d, J=10.27 Hz, 1H), 7.71 (d, J=6.85 Hz, 1H), 7.66 (d, J=10.03 Hz, 1H), 7.39 (d, J=8.19 Hz, 2H), 7.02 (br. s., 1H), 6.96 (d, J=8.68 Hz, 2H), 6.23 (s, 2H), 3.14 (br. s., 4H), 2.63-2.71 (m, 1H), 2.57 (d, J=4.03 Hz, 4H), 2.23 (s, 3H), 1.00 (d, J=6.48 Hz, 6 H); LCMS [M+H]⁺=490.40.

### Example 525: 6-(6′-amino-6-(azetidin-1-ylmethyl)-2-fluoro-5-morpholino-[2,3′-bipyridin]-5′-yl)-3,4-dihydroisoquinolin-1(2H)-one (I-578)

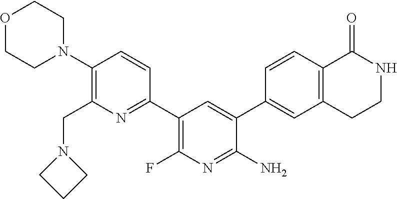 [SMILES: Nc1nc(F)c(-c2ccc(N3CCOCC3)c(CN3CCC3)n2)cc1-c1ccc2c(c1)CCNC2=O]

Prepared from 4-(2-(azetidin-1-ylmethyl)-6-bromopyridin-3-yl)morpholine (60 mg, 0.173 mmol, in turn made from 6-bromo-3-morpholinopicolinaldehyde and azetidine using procedures analogous to earlier examples) and 6-(2-amino-6-fluoro-5-(4,4,5,5-tetramethyl-1,3,2-dioxaborolan-2-yl)pyridin-3-yl)-3,4-dihydroisoquinolin-1(2H)-one (86 mg, 0.225 mmol) to yield the title compound as a white solid (28 mg, 32% yield). ¹H NMR (500 MHz, DMSO-d₆) δ=8.18-8.07 (m, 1H), 8.02-7.98 (m, 1H), 7.97-7.91 (m, 1H), 7.61-7.55 (m, 1H), 7.58-7.51 (m, 1H), 7.48-7.41 (m, 2H), 6.56-6.39 (m, 2H) 3.80-3.74 (m, 4H), 3.73-3.64 (m, 2H), 3.46-3.40 (m, 2H), 3.39-3.34 (m, 4H), 3.03-2.94 (m, 6H), 2.02-1.93 (m, 2H). LCMS: [M+H]⁺=489.64.

### Example 526: 6-(6′-amino-6-(azetidin-1-ylmethyl)-2-fluoro-5-morpholino-[2,3′-bipyridin]-5′-yl)-7-fluoro-3,4-dihydroisoquinolin-1(2H)-one (I-579)

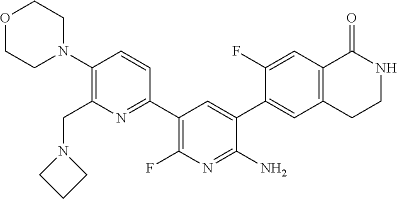 [SMILES: Nc1nc(F)c(-c2ccc(N3CCOCC3)c(CN3CCC3)n2)cc1-c1cc2c(cc1F)C(=O)NCC2]

Prepared from 4-(2-(azetidin-1-ylmethyl)-6-bromopyridin-3-yl)morpholine (60 mg, 0.173 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (90 mg, 0.225 mmol) to yield the title compound as an off white solid (32 mg, 35% yield). ¹H NMR (500 MHz, DMSO-d₆) δ=8.18-8.07 (m, 2H), 7.68-7.59 (m, 2H), 7.55-7.49 (m, 1H), 7.45-7.38 (m, 1H), 6.56-6.39 (m, 2H), 3.87-3.69 (m, 6H), 3.47-3.39 (m, 4H), 3.39-3.37 (m, 2H), 3.03-2.96 (m, 4H), 2.95-2.91 (m, 2H), 2.13-1.91 (m, 2H); LCMS: [M+H]⁺=507.57.

### Example 527: 6-(6′-amino-6-((ethyl(methyl)amino)methyl)-2′-fluoro-5-morpholino-[2,3′-bipyridin]-5′-yl)-3,4-dihydroisoquinolin-1(2H)-one (I-580)

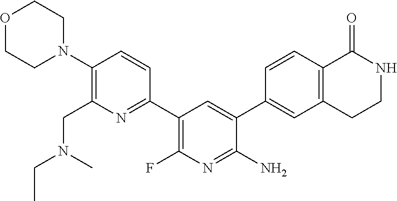 [SMILES: CCN(C)Cc1nc(-c2cc(-c3ccc4c(c3)CCNC4=O)c(N)nc2F)ccc1N1CCOCC1]

Prepared from N-((6-bromo-3-morpholinopyridin-2-yl)methyl)-N-methylethanamine (60 mg, 0.173 mmol, in turn made from 6-bromo-3-morpholinopicolinaldehyde and N-ethylmethylamine analogous to earlier examples) and 6-(2-amino-6-fluoro-5-(4,4,5,5-tetramethyl-1,3,2-dioxaborolan-2-yl)pyridin-3-yl)-3,4-dihydroisoquinolin-1(2H)-one (86 mg, 0.223 mmol) to yield the title compound as an off white solid (80 mg, 90% yield). ¹H NMR (500 MHz, DMSO-d₆) δ 8.0-8.1 (m, 1H), 7.9-8.0 (m, 2H), 7.6-7.7 (m, 1H), 7.5-7.6 (m, 1H), 7.4-7.4 (m, 2H), 6.4-6.5 (m, 2H), 3.7-3.8 (m, 4H), 3.6-3.7 (m, 2H), 3.4-3.5 (m, 2H), 3.0-3.1 (m, 4H), 2.9-3.0 (m, 2H), 2.55 (br s, 2H), 2.24 (br s, 3H), 0.9-1.0 (m, 3H); LCMS: [M+H]⁺=491.59.

### Example 528: 6-(6′-amino-6-((ethyl(methyl)amino)methyl)-2′-fluoro-5-morpholino-[2,3′-bipyridin]-5′-yl)-7-fluoro-3,4-dihydroisoquinolin-1(2H)-one (I-581)

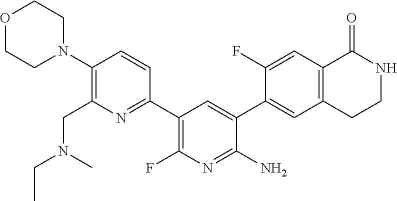 [SMILES: CCN(C)Cc1nc(-c2cc(-c3cc4c(cc3F)C(=O)NCC4)c(N)nc2F)ccc1N1CCOCC1]

Prepared from N-((6-bromo-3-morpholinopyridin-2-yl)methyl)-N-methylethanamine (60 mg, 0.172 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (90 mg, 0.223 mmol) to yield the title compound as a beige solid (68 mg, 74% yield). ¹H NMR (500 MHz, DMSO-d₆) δ=8.1-8.2 (m, 1H), 8.0-8.1 (m, 1H), 7.6-7.7 (m, 2H), 7.5-7.6 (m, 1H), 7.4-7.4 (m, 1H), 6.4-6.5 (m, 2H), 3.7-3.8 (m, 4H), 3.62 (br s, 2H), 3.4-3.5 (m, 2H), 3.0-3.1 (m, 4H), 2.9-3.0 (m, 2H), 2.5-2.6 (m, 2H), 2.1-2.3 (m, 3H), 0.9-1.0 (m, 3H); LCMS: [M+H]⁺=509.57.

### Example 529: 6-(6′-amino-2′-fluoro-6-(((2-methoxyethyl)(methyl)amino)methyl)-5-morpholino-[2,3′-bipyridin]-5′-yl)-3,4-dihydroisoquinolin-1(2H)-one (I-582)

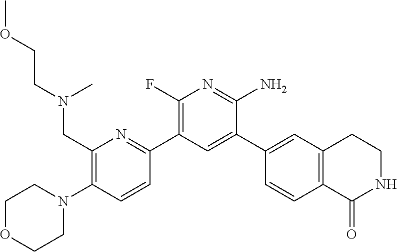 [SMILES: COCCN(C)Cc1nc(-c2cc(-c3ccc4c(c3)CCNC4=O)c(N)nc2F)ccc1N1CCOCC1]

Prepared from N-((6-bromo-3-morpholinopyridin-2-yl)methyl)-2-methoxy-N-methylethan-1-amine (60 mg, 0.157 mmol, in turn made from 6-bromo-3-morpholinopicolinaldehyde and N-(2-methoxyethyl)methylamine as per previous examples) and 6-(2-amino-6-fluoro-5-(4,4,5,5-tetramethyl-1,3,2-dioxaborolan-2-yl)pyridin-3-yl)-3,4-dihydroisoquinolin-1(2H)-one (78 mg, 0.204 mmol) to yield the title compound as an off white solid (45 mg, 52% yield). ¹H NMR (500 MHz, DMSO-d₆) δ 8.1-8.1 (m, 1H), 7.9-8.0 (m, 2H), 7.6-7.7 (m, 1H), 7.5-7.6 (m, 1H), 7.4-7.5 (m, 2H), 6.4-6.5 (m, 2H), 3.7-3.8 (m, 4H), 3.69 (s, 2H), 3.4-3.4 (m, 4H), 3.1-3.2 (m, 3H), 3.0-3.1 (m, 4H), 2.9-3.0 (m, 2H), 2.7-2.7 (m, 2H), 2.3-2.3 (m, 3H); LCMS: [M+H]⁺=521.67.

### Example 530: 6-(6′-amino-2′-fluoro-6-(((2-methoxyethyl)(methyl)amino)methyl)-5-morpholino-[2,3′-bipyridin]-5′-yl)-7-fluoro-3,4-dihydroisoquinolin-1(2H)-one (I-583)

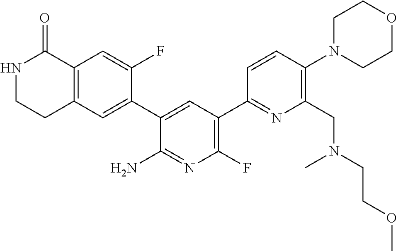 [SMILES: COCCN(C)Cc1nc(-c2cc(-c3cc4c(cc3F)C(=O)NCC4)c(N)nc2F)ccc1N1CCOCC1]

Prepared from N-((6-bromo-3-morpholinopyridin-2-yl)methyl)-2-methoxy-N-methylethan-1-amine (60 mg, 0.157 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (82 mg, 0.204 mmol) to yield the title compound as a pale-yellow solid (52 mg, 59% yield). ¹H NMR (500 MHz, DMSO-d₆) δ=8.1-8.2 (m, 1H), 8.1-8.1 (m, 1H), 7.6-7.7 (m, 2H), 7.5-7.6 (m, 1H), 7.4-7.4 (m, 1H), 6.4-6.6 (m, 2H), 3.7-3.8 (m, 4H), 3.68 (br s, 2H), 3.4-3.4 (m, 4H), 3.1-3.2 (m, 3H), 3.0-3.1 (m, 4H), 2.9-3.0 (m, 2H), 2.6-2.7 (m, 2H), 2.2-2.3 (m, 3H); LCMS: [M+H]⁺=539.66.

### Example 531: (S)-6-(6′-amino-2-fluoro-6-((3-methoxypyrrolidin-1-yl)methyl)-5-morpholino-[2,3′-bipyridin]-5′-yl)-3,4-dihydroisoquinolin-1(2H)-one (I-584)

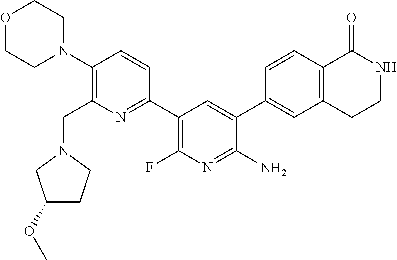 [SMILES: CO[C@H]1CCN(Cc2nc(-c3cc(-c4ccc5c(c4)CCNC5=O)c(N)nc3F)ccc2N2CCOCC2)C1]

Prepared from (S)-4-(6-bromo-2-((3-methoxypyrrolidin-1-yl)methyl)pyridin-3-yl)morpholine and 6-(2-amino-6-fluoro-5-(4,4,5,5-tetramethyl-1,3,2-dioxaborolan-2-yl)pyridin-3-yl)-3,4-dihydroisoquinolin-1(2H)-one to yield the title compound as a white solid (73% yield). ¹H NMR (500 MHz, DMSO-d₆) δ=8.14-8.07 (m, 1H), 7.99-7.95 (m, 1H), 7.94-7.90 (m, 1H), 7.66-7.61 (m, 1H), 7.55-7.50 (m, 1H), 7.45-7.38 (m, 2H), 6.53-6.32 (m, 2H), 3.86-3.81 (m, 1H), 3.80-3.75 (m, 4H), 3.75-3.65 (m, 2H), 3.44-3.39 (m, 2H), 3.12-3.07 (m, 3H), 3.07-2.99 (m, 4H), 2.96 (br t, J=6.5 Hz, 2H), 2.88-2.79 (m, 1H), 2.75-2.68 (m, 1H), 2.67-2.63 (m, 1H), 2.48-2.45 (m, 1H), 1.97-1.87 (m, 1H), 1.65-1.56 (m, 1H); LCMS: [M+H]⁺=533.58.

### Example 532: (S)-6-(6′-amino-2-fluoro-6-((3-methoxypyrrolidin-1-yl)methyl)-5-morpholino-[2,3′-bipyridin]-5′-yl)-7-fluoro-3,4-dihydroisoquinolin-1(2H)-one (I-585)

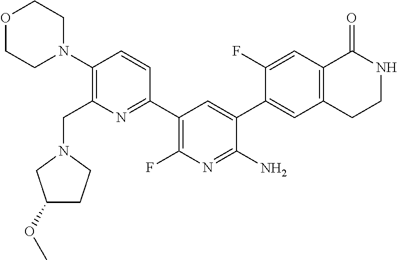 [SMILES: CO[C@H]1CCN(Cc2nc(-c3cc(-c4cc5c(cc4F)C(=O)NCC5)c(N)nc3F)ccc2N2CCOCC2)C1]

Prepared from (S)-4-(6-bromo-2-((3-methoxypyrrolidin-1-yl)methyl)pyridin-3-yl)morpholine (60 mg, 0.152 mmol) from Example 57, step 1 with 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (79 mg, 0.197 mmol) to yield the title compound (77 mg, 88% yield). ¹H NMR (500 MHz, DMSO-d₆) δ=8.16-8.11 (m, 1H), 8.10-8.05 (m, 1H), 7.65-7.60 (m, 2H), 7.55-7.50 (m, 1H), 7.44-7.39 (m, 1H), 6.53-6.41 (m, 2H), 3.84-3.80 (m, 1H), 3.79-3.75 (m, 4H), 3.74-3.69 (m, 2H), 3.41 (dt, J=2.6, 6.4 Hz, 2H), 3.10-3.07 (m, 3H), 3.06-2.98 (m, 4H), 2.94-2.90 (m, 2H), 2.83-2.78 (m, 1H), 2.71-2.65 (m, 1H), 2.62 (br dd, J=3.1, 10.3 Hz, 1H), 2.57-2.54 (m, 1H), 1.95-1.87 (m, 1H), 1.62-1.54 (m, 1H); LCMS: [M+H]⁺=551.63.

### Example 533: 6-(2-amino-6-fluoro-5-(4-(tetrahydro-2H-pyran-4-yl)phenyl)pyridin-3-yl)-4-methylisoquinolin-1(2H)-one (I-586)

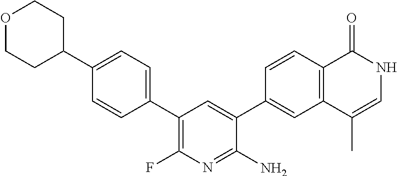 [SMILES: Cc1c[nH]c(=O)c2ccc(-c3cc(-c4ccc(C5CCOCC5)cc4)c(F)nc3N)cc12]

Prepared from 6-(2-amino-5-bromo-6-fluoropyridin-3-yl)-4-methylisoquinolin-1(2H)-one (50 mg, 0.144 mmol) and 4-(4-tetrahydropyranyl)phenylboronic acid pinacol ester (49.7 mg, 0.172 mmol) to give the title compound (54.2 mg, 88% yield) as a white solid. ¹H NMR (500 MHz, DMSO-d₆) δ ppm 11.09 (d, J=5.62 Hz, 1H), 8.28 (d, J=8.31 Hz, 1H), 7.69-7.74 (m, 2H), 7.63 (dd, J=8.31, 1.35 Hz, 1H), 7.50 (d, J=7.46 Hz, 2H), 7.31 (d, J=8.31 Hz, 2H), 7.04 (d, J=5.26 Hz, 1H), 6.34 (s, 2H), 3.92-3.98 (m, 2H), 3.44 (td, J=10.97, 3.48 Hz, 2H), 2.79 (tt, J=10.42, 5.23 Hz, 1H), 2.22-2.25 (m, 3H), 1.63-1.74 (m, 4H); LCMS: [M+H]⁺=430.42.

### Example 534: 6-(2-amino-6-fluoro-5-(4-(4-isopropylpiperazin-1-yl)phenyl)pyridin-3-yl)-7-fluoro-3-methylisoquinolin-1(2H)-one (I-587)

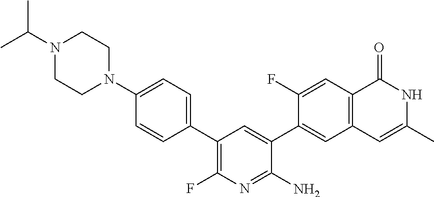 [SMILES: Cc1cc2cc(-c3cc(-c4ccc(N5CCN(C(C)C)CC5)cc4)c(F)nc3N)c(F)cc2c(=O)[nH]1]

Step 1: (7-fluoro-3-methyl-1-oxo-1,2-dihydroisoquinolin-6-yl)boronic acid

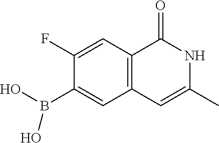 [SMILES: Cc1cc2cc(B(O)O)c(F)cc2c(=O)[nH]1]

Prepared from 6-bromo-7-fluoro-3-methylisoquinolin-1(2H)-one (700 mg, 2.73 mmol) to give the boronic acid which was used in the next step without further purification. LCMS: [M−H]−=220.10.

Step 2: 6-(2-amino-5-bromo-6-fluoropyridin-3-yl)-7-fluoro-3-methylisoquinolin-1(2H)-one

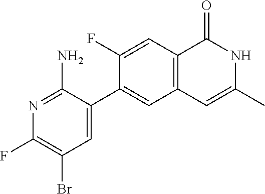 [SMILES: Cc1cc2cc(-c3cc(Br)c(F)nc3N)c(F)cc2c(=O)[nH]1]

Prepared from 5-bromo-6-fluoro-3-iodopyridin-2-amine (350 mg, 1.104 mmol) and (7-fluoro-3-methyl-1-oxo-1,2-dihydroisoquinolin-6-yl)boronic acid (293 mg, 1.325 mmol) to the product (99 mg, 24.5% yield) as a beige solid. LCMS: [M+H]⁺=366.29.

Step 3: 6-(2-amino-6-fluoro-5-(4-(4-isopropylpiperazin-1-yl)phenyl)pyridin-3-yl)-7-fluoro-3-methylisoquinolin-1(2H)-one

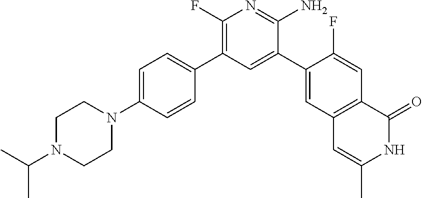 [SMILES: Cc1cc2cc(-c3cc(-c4ccc(N5CCN(C(C)C)CC5)cc4)c(F)nc3N)c(F)cc2c(=O)[nH]1]

Prepared from 6-(2-amino-5-bromo-6-fluoropyridin-3-yl)-7-fluoro-3-methylisoquinolin-1(2H)-one (30 mg, 0.082 mmol) and 4-(4-isopropylpiperazinyl)phenylboronic acid, pinacol ester (32.5 mg, 0.098 mmol) to give the title compound (15.8 mg, 39.4% yield) as a beige solid. ¹H NMR (500 MHz, DMSO-d₆) δ ppm 11.38 (s, 1H), 7.84 (d, J=10.27 Hz, 1H), 7.65 (d, J=6.97 Hz, 1H), 7.61 (d, J=10.15 Hz, 1H), 7.38 (d, J=8.31 Hz, 2H), 6.96 (d, J=8.68 Hz, 2H), 6.37 (s, 1H), 6.19 (s, 2H), 3.11-3.17 (m, 4H), 2.66 (dd, J=13.27, 6.79 Hz, 1H), 2.55-2.59 (m, 4H), 2.22 (s, 3H), 1.00 (d, J=6.48 Hz, 6H); LCMS: [M+H]⁺=490.58.

### Example 535: 6-(6′-amino-6-((dimethylamino)methyl)-2′-fluoro-5-morpholino-[2,3′-bipyridin]-5′-yl)-4-methylisoquinolin-1(2H)-one (I-588)

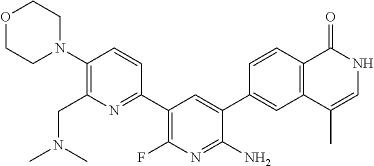 [SMILES: Cc1c[nH]c(=O)c2ccc(-c3cc(-c4ccc(N5CCOCC5)c(CN(C)C)n4)c(F)nc3N)cc12]

Step 1: 6-(2-amino-6-fluoro-5-(4,4,5,5-tetramethyl-1,3,2-dioxaborolan-2-yl)pyridin-3-yl)-4-methylisoquinolin-1(2H)-one

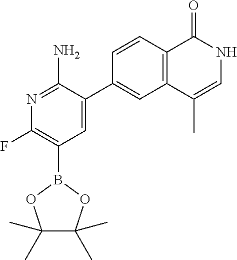 [SMILES: Cc1c[nH]c(=O)c2ccc(-c3cc(B4OC(C)(C)C(C)(C)O4)c(F)nc3N)cc12]

Prepared from 6-(2-amino-5-bromo-6-fluoropyridin-3-yl)-4-methylisoquinolin-1(2H)-one (53 mg, 0.152 mmol) to give a mixture of the boronate and boronic acid which was used in the next step without further purification. LCMS [M+H]+ 270.38 Boronic acid, Boronate 396.44.

Step 2: 6-(6′-amino-6-((dimethylamino)methyl)-2′-fluoro-5-morpholino-[2,3′-bipyridin]-5′-yl)-4-methylisoquinolin-1(2H)-one

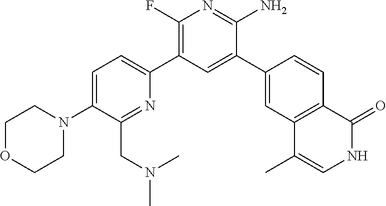 [SMILES: Cc1c[nH]c(=O)c2ccc(-c3cc(-c4ccc(N5CCOCC5)c(CN(C)C)n4)c(F)nc3N)cc12]

Prepared from 6-(2-amino-6-fluoro-5-(4,4,5,5-tetramethyl-1,3,2-dioxaborolan-2-yl)pyridin-3-yl)-4-methylisoquinolin-1(2H)-one (59.9 mg, 0.152 mmol) and 1-(6-bromo-3-morpholinopyridin-2-yl)-N,N-dimethylmethanamine (35 mg, 0.117 mmol) to give the title compound (11.6 mg, 20.4% yield) as a beige solid. ¹H NMR (500 MHz, DMSO-d₆) δ ppm 11.11 (d, J=5.38 Hz, 1H), 8.30 (d, J=8.19 Hz, 1H), 8.15 (d, J=10.03 Hz, 1H), 7.69 (s, 1H), 7.65 (d, J=8.19 Hz, 1H), 7.59 (d, J=8.31 Hz, 1H), 7.54 (d, J=8.56 Hz, 1H), 7.05 (d, J=5.50 Hz, 1H), 6.51 (s, 2H), 3.77 (br. s., 4H), 3.52 (s, 2H), 3.05 (br. s., 4H), 2.24 (s, 6H), 2.23 (s, 3H); LCMS: [M+H]⁺=489.46.

### Example 536: 6-(6′-amino-6-((dimethylamino)methyl)-2′-fluoro-5-morpholino-[2,3′-bipyridin]-5′-yl)-4-fluoroisoquinolin-1(2H)-one (I-589)

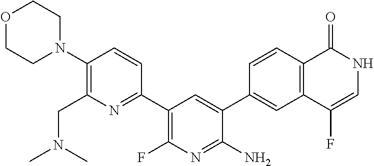 [SMILES: CN(C)Cc1nc(-c2cc(-c3ccc4c(=O)[nH]cc(F)c4c3)c(N)nc2F)ccc1N1CCOCC1]

Step 1: 6-(2-amino-6-fluoro-5-(4,4,5,5-tetramethyl-1,3,2-dioxaborolan-2-yl)pyridin-3-yl)-4-fluoroisoquinolin-1(2H)-one

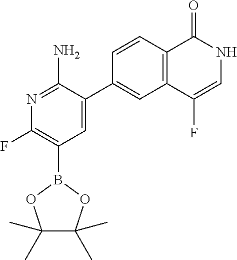 [SMILES: CC1(C)OB(c2cc(-c3ccc4c(=O)[nH]cc(F)c4c3)c(N)nc2F)OC1(C)C]

Prepared from 6-(2-amino-5-bromo-6-fluoropyridin-3-yl)-4-fluoroisoquinolin-1(2H)-one (53 mg, 0.151 mmol) to give a mixture of the boronate and boronic acid which was used in the next step without further purification. LCMS: [M+H]+ 318.34 Boronic acid, Boronate 400.45.

Step 2: 6-(6′-amino-6-((dimethylamino)methyl)-2′-fluoro-5-morpholino-[2,3′-bipyridin]-5′-yl)-4-fluoroisoquinolin-1(2H)-one

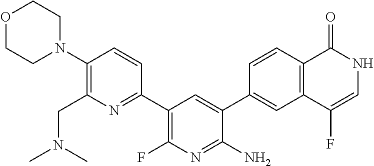 [SMILES: CN(C)Cc1nc(-c2cc(-c3ccc4c(=O)[nH]cc(F)c4c3)c(N)nc2F)ccc1N1CCOCC1]

Prepared from 6-(2-amino-6-fluoro-5-(4,4,5,5-tetramethyl-1,3,2-dioxaborolan-2-yl)pyridin-3-yl)-4-fluoroisoquinolin-1(2H)-one (60.5 mg, 0.152 mmol) and 1-(6-bromo-3-morpholinopyridin-2-yl)-N,N-dimethylmethanamine (35 mg, 0.117 mmol) to give the title compound (13.6 mg, 23.7% yield) as a beige solid. ¹H NMR (500 MHz, DMSO-d₆) δ ppm 11.17 (br. s., 1H), 8.29 (d, J=8.19 Hz, 1H), 8.13 (d, J=9.90 Hz, 1H), 7.77 (s, 1H), 7.68 (d, J=8.19 Hz, 1H), 7.65 (d, J=8.07 Hz, 1H), 7.54 (d, J=8.44 Hz, 1H), 7.43 (br. s., 1H), 6.55 (s, 2H), 3.77 (br. s., 4H), 3.52 (s, 2H), 3.05 (br. s., 4H), 2.24 (s, 6H); LCMS: [M+H]⁺=493.53.

### Example 537: tert-butyl4-(6′-amino-2′-fluoro-5′-(1-oxo-1,2,3,4-tetrahydroisoquinolin-6-yl)-[2,3′-bipyridin]-5-yl) piperazine-1-carboxylate (1-590)

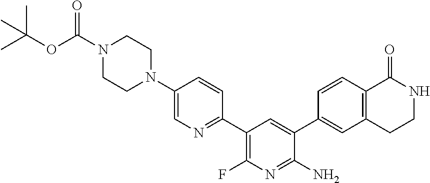 [SMILES: CC(C)(C)OC(=O)N1CCN(c2ccc(-c3cc(-c4ccc5c(c4)CCNC5=O)c(N)nc3F)nc2)CC1]

A microwave vial containing a mixture of XPhos Pd G2 (14.80 mg, 0.019 mmol), tert-butyl 4-(6-chloropyridin-3-yl)piperazine-1-carboxylate (80 mg, 0.269 mmol), 6-(2-amino-6-fluoro-5-(4,4,5,5-tetramethyl-1,3,2-dioxaborolan-2-yl)pyridin-3-yl)-3,4-dihydroisoquinolin-1(2H)-one (124 mg, 0.322 mmol) and K₂CO₃ (114 mg, 0.537 mmol) in t-butanol (3.5 ml) was flushed with Argon. Water (1. ml) was added and the reaction mixture was heated at 1000C in an oil bath at 100° C. The reaction mixture was concentrated onto celite and purified by silica gel chromatograpy, eluting with dichloromethane containing 0-1.5% MeOH and 0-0.15% NH4OH to afford the title compound as a cream colored solid. (123 mg, 84% yield). ¹H NMR (500 MHz, DMSO-d₆) δ=8.40-8.34 (m, 1H), 8.12-8.05 (m, 1H), 7.98-7.94 (m, 1H), 7.94-7.90 (m, 1H), 7.66-7.58 (m, 1H), 7.46-7.37 (m, 3H), 6.53-6.26 (m, 2H), 3.56-3.48 (m, 4H), 3.44-3.41 (m, 2H), 3.24-3.16 (m, 4H), 3.00-2.94 (m, 2H), 1.47-1.40 (m, 9H); LCMS: [M+H]⁺=519.54

### Example 538: 6-(5-(4-acetylpiperazin-1-yl)-6′-amino-2′-fluoro-[2,3′-bipyridin]-5′-yl)-3,4-dihydroisoquinolin-1(2H)-one (I-591)

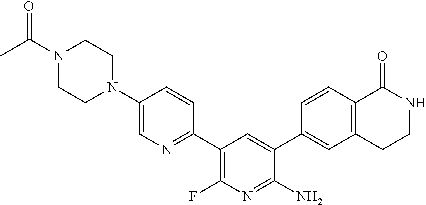 [SMILES: CC(=O)N1CCN(c2ccc(-c3cc(-c4ccc5c(c4)CCNC5=O)c(N)nc3F)nc2)CC1]

Step 1: 6-(6′-amino-2′-fluoro-5-(piperazin-1-yl)-[2,3′-bipyridin]-5′-yl)-3,4-dihydroisoquinolin-9(2H)-one

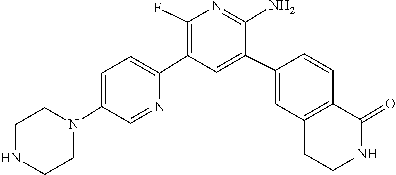 [SMILES: Nc1nc(F)c(-c2ccc(N3CCNCC3)cn2)cc1-c1ccc2c(c1)CCNC2=O]

Prepared by deprotecting tert-butyl 4-(6′-amino-2′-fluoro-5′-(1-oxo-1,2,3,4-tetrahydroisoquinolin-6-yl)-[2,3′-bipyridin]-5-yl)piperazine-1-carboxylate (112 mg, 0.216 mmol) with TFA (1.488 ml, 19.44 mmol). The title compound was isolated as a cream colored solid (84 mg, 93% yield); LCMS: [M+H]⁺=419.45

Step 2: 6-(5-(4-acetylpiperazin-1-yl)-6′-amino-2′-fluoro-[2,3′-bipyridin]-5′-yl)-3,4-dihydroisoquinolin-1(2H)-one

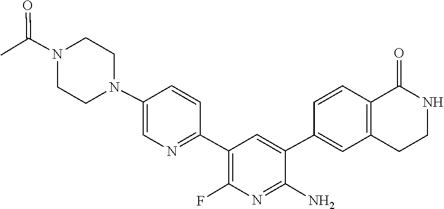 [SMILES: CC(=O)N1CCN(c2ccc(-c3cc(-c4ccc5c(c4)CCNC5=O)c(N)nc3F)nc2)CC1]

To a suspension of 6-(6′-amino-2′-fluoro-5-(piperazin-1-yl)-[2,3′-bipyridin]-5′-yl)-3,4-dihydroisoquinolin-1(2H)-one (45 mg, 0.108 mmol) and N,N-Diisopropylethylamine (0.037 ml, 0.215 mmol) in dichloromethane (5 ml) at RT, was added acetic anhydride (10.16 μl, 0.108 mmol) and the reaction mixture was stirred at RT for 60 minutes. The solid was filtered, the filter cake was washed with small volume of DCM (x3) and dried to isolate the title compound as a pale-yellow solid (41 mg, 79% yield). ¹H NMR (500 MHz, DMSO-d₆) δ 8.3-8.4 (m, 1H), 8.0-8.1 (m, 1H), 7.9-8.0 (m, 1H), 7.9-7.9 (m, 1H), 7.6-7.7 (m, 1H), 7.4-7.5 (m, 3H), 6.3-6.5 (m, 2H), 3.6-3.7 (m, 4H), 3.4-3.4 (m, 2H), 3.2-3.3 (m, 2H), 3.2-3.2 (m, 2H), 2.97 (br t, 2H, J=6.4 Hz), 2.05 (s, 3H); LCMS: [M+H]⁺=461.44

### Example 539: 6-(5-(4-acetylpiperazin-1-yl)-6′-amino-2′-fluoro-[2,3′-bipyridin]-5′-yl)-7-fluoro-3,4-dihydroisoquinolin-1(2H)-one (I-592)

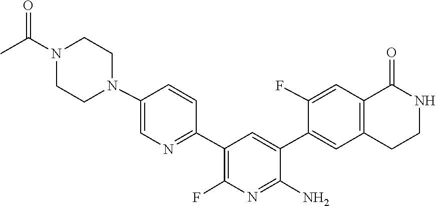 [SMILES: CC(=O)N1CCN(c2ccc(-c3cc(-c4cc5c(cc4F)C(=O)NCC5)c(N)nc3F)nc2)CC1]

Step 1: tert-butyl 4-(6′-amino-2′-fluoro-5′-(7-fluoro-1-oxo-1,2,3,4-tetrahydroisoquinolin-6-yl)-[2,3′-bipyridin]-5-yl)piperazine-1-carboxylate

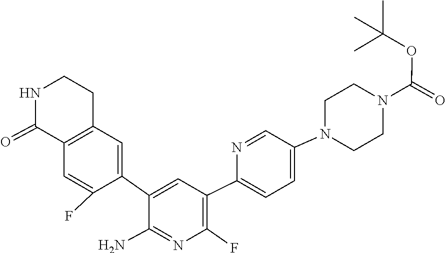 [SMILES: CC(C)(C)OC(=O)N1CCN(c2ccc(-c3cc(-c4cc5c(cc4F)C(=O)NCC5)c(N)nc3F)nc2)CC1]

Prepared from tert-butyl 4-(6-chloropyridin-3-yl)piperazine-1-carboxylate (80 mg, 0.269 mmol) with 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (129 mg, 0.322 mmol) to give the product (54 mg, 22.5% yield). LCMS: [M+H]⁺=537.53

Step 2: 6-(6′-amino-2′-fluoro-5-(piperazin-1-yl)-[2,3′-bipyridin]-5′-yl)-7-fluoro-3,4-dihydroisoquinolin-1(2H)-one

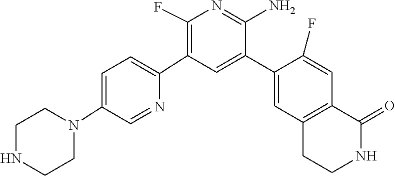 [SMILES: Nc1nc(F)c(-c2ccc(N3CCNCC3)cn2)cc1-c1cc2c(cc1F)C(=O)NCC2]

Prepared by deprotecting tert-butyl 4-(6′-amino-2′-fluoro-5′-(7-fluoro-1-oxo-1,2,3,4-tetrahydroisoquinolin-6-yl)-[2,3′-bipyridin]-5-yl)piperazine-1-carboxylate (54 mg, 0.101 mmol) with TFA (0.694 ml, 9.06 mmol) to give product (34 mg, 53% yield) LCMS: [M +H]⁺=437.37

Step 3: 6-(5-(4-acetylpiperazin-1-yl)-6′-amino-2′-fluoro-[2,3′-bipyridin]-5′-yl)-7-fluoro-3,4-dihydroisoquinolin-1(2H)-one

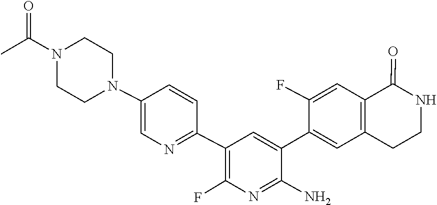 [SMILES: CC(=O)N1CCN(c2ccc(-c3cc(-c4cc5c(cc4F)C(=O)NCC5)c(N)nc3F)nc2)CC1]

Prepared from 6-(6′-amino-2′-fluoro-5-(piperazin-1-yl)-[2,3′-bipyridin]-5′-yl)-7-fluoro-3,4-dihydroisoquinolin-1(2H)-one (39 mg, 0.062 mmol) and acetic anhydride (5.83 μl, 0.062 mmol) to afford title compound as an off white solid (23 mg, 74% yield). ¹H NMR (500 MHz, DMSO-d₆) δ 8.3-8.4 (m, 1H), 8.1-8.2 (m, 1H), 8.0-8.1 (m, 1H), 7.6-7.7 (m, 2H), 7.4-7.5 (m, 2H), 6.3-6.5 (m, 2H), 3.6-3.7 (m, 4H), 3.4-3.5 (m, 2H), 3.2-3.3 (m, 2H), 3.19 (br d, 2H, J=2.6 Hz), 2.94 (br d, 2H, J=4.0 Hz), 2.05 (br d, 3H, J=4.2 Hz); LCMS: [M+H]⁺=479.49

### Example 540: 6-(6′-amino-5-(4-(4,4-difluorobutyl)piperazin-1-yl)-2′-fluoro-[2,3′-bipyridin]-5′-yl)-3,4-dihydroisoquinolin-1(2H)-one (I-593)

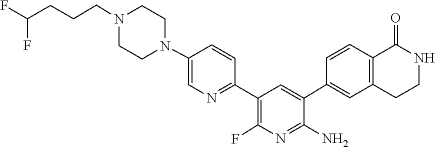 [SMILES: Nc1nc(F)c(-c2ccc(N3CCN(CCCC(F)F)CC3)cn2)cc1-c1ccc2c(c1)CCNC2=O]

Prepared from 6-(6′-amino-2′-fluoro-5-(piperazin-1-yl)-[2,3′-bipyridin]-5′-yl)-3,4-dihydroisoquinolin-1(2H)-one (39 mg, 0.093 mmol) and 4-bromo-1,1-difluorobutane (32.2 mg, 0.186 mmol) to give the title compound as a pale-yellow solid (39 mg, 78% yield). ¹H NMR (DMSO-d₆, 500 MHz) δ 8.3-8.4 (m, 1H), 8.1-8.1 (m, 1H), 7.9-8.0 (m, 2H), 7.6-7.6 (m, 1H), 7.4-7.5 (m, 1H), 7.4-7.4 (m, 1H), 7.4-7.4 (m, 1H), 6.3-6.4 (m, 2H), 6.0-6.2 (m, 1H), 3.4-3.5 (m, 2H), 3.2-3.3 (m, 4H), 3.0-3.0 (m, 2H), 2.5-2.5 (m, 4H), 2.3-2.4 (m, 2H), 1.8-1.9 (m, 2H), 1.5-1.7 (m, 2H); LCMS: [M+H]⁺=511.45.

### Example 541: 6-(2-amino-5-(3-((dimethylamino)methyl)phenyl)-6-fluoropyridin-3-yl)-3,4-dihydroisoquinolin-1(2H)-one (I-594)

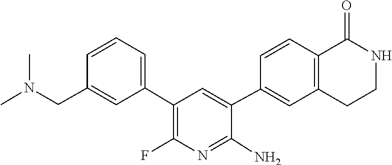 [SMILES: CN(C)Cc1cccc(-c2cc(-c3ccc4c(c3)CCNC4=O)c(N)nc2F)c1]

Prepared from (3-bromobenzyl)dimethylamine (50 mg, 0.234 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (116 mg, 0.304 mmol) to afford the title compound as a beige solid (43 mg, 45% yield). ¹H NMR (500 MHz, DMSO-d₆) δ ppm 7.95 (br. s., 1H), 7.92 (d, J=7.95 Hz, 1H), 7.64 (d, J=10.03 Hz, 1H), 7.42-7.51 (m, 4H), 7.37 (t, J=7.52 Hz, 1H), 7.23 (d, J=7.34 Hz, 1H), 6.19-6.42 (m, 2H), 3.42 (br. s., 2H), 3.38-3.41 (m, 2H), 2.96 (t, J=6.24 Hz, 2H), 2.16 (s, 6H); LCMS: [M+H]⁺=391.43.

### Example 542: 7-(2-amino-5-(3-((dimethylamino)methyl)-4-((2R,6S)-2,6-dimethylmorpholino)phenyl)-6-fluoropyridin-3-yl)-2-methylquinazolin-4(3H)-one (I-595)

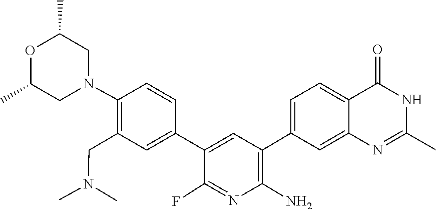

Prepared from 7-(2-amino-5-bromo-6-fluoropyridin-3-yl)-2-methylquinazolin-4(3H)-one (40 mg, 0.115 mmol) and 1-(2-(cis-2,6-dimethylmorpholino)-5-(4,4,5,5-tetramethyl-1,3,2-dioxaborolan-2-yl)phenyl)-N,N-dimethylmethanamine hydrochloride (47.1 mg, 0.115 mmol) to give the title compound (41 mg 68.5% yield). ¹H NMR (500 MHz, DMSO-d₆) δ=12.23 (s, 1H), 8.19-8.06 (m, 1H), 7.66 (s, 1H), 7.64 (d, J=10.0 Hz, 1H), 7.57 (d, J=8.3 Hz, 1H), 7.53 (s, 1H), 7.45 (br d, J=8.8 Hz, 1H), 7.11 (br d, J=8.4 Hz, 1H), 6.30 (s, 2H), 3.82-3.73 (m, 2H), 3.12 (br d, J=11.4 Hz, 2H), 2.39-2.31 (m, 5H), 2.25 (br s, 6H), 1.10 (d, J=6.2 Hz, 6H); LCMS: [M+H]⁺=517.60.

### Example 543: 6-(2-amino-5-(3-((dimethylamino)methyl)-4-((2R,6S)-2,6-dimethylmorpholino)phenyl)-6-fluoropyridin-3-yl)-8-fluoro-3,4-dihydroisoquinolin-1(2H)-one (I-596)

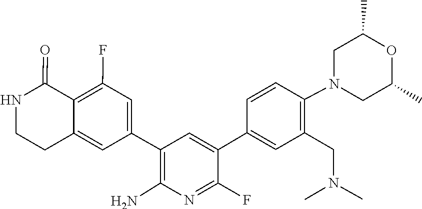 [SMILES: C[C@@H]1CN(c2ccc(-c3cc(-c4cc(F)c5c(c4)CCNC5=O)c(N)nc3F)cc2CN(C)C)C[C@H](C)O1]

Prepared from 6-(2-amino-5-bromo-6-fluoropyridin-3-yl)-8-fluoro-3,4-dihydroisoquinolin-1(2H)-one (40 mg, 0.113 mmol) and 1-(2-((2S,6R)-2,6-dimethylmorpholino)-5-(4,4,5,5-tetramethyl-1,3,2-dioxaborolan-2-yl)phenyl)-N,N-dimethylmethanamine hydrochloride (46.4 mg, 0.113 mmol) to give the title compound (25.3 mg, 42.4% yield). ¹H NMR (500 MHz, DMSO-d₆) δ=7.99 (br. s., 1H), 7.63 (d, J=10.03 Hz, 1H), 7.53 (br. s., 1H), 7.43 (br. s., 1H), 7.28 (s, 1H), 7.25 (d, J=12.10 Hz, 1H), 7.12 (br. s, 1H), 6.37 (br. s., 2H), 3.73-3.82 (m, 2H), 3.30-3.55 (m, 4H), 3.10 (d, J=8.44 Hz, 2H), 2.93 (t, J=6.11 Hz, 2H), 2.12-2.44 (m, 8H), 1.11 (d, J=6.11 Hz, 6H); LCMS: [M+H]⁺=522.55.

### Example 544: 6-(2-amino-5-(3-((dimethylamino)methyl)-4-((2R,6S)-2,6-dimethylmorpholino)phenyl)-6-fluoropyridin-3-yl)-7-fluoro-3,4-dihydroisoquinolin-1(2H)-one (I-597)

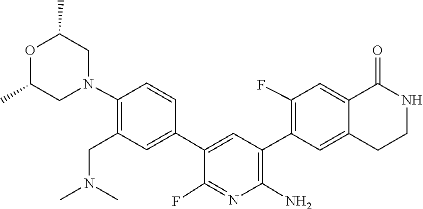 [SMILES: C[C@@H]1CN(c2ccc(-c3cc(-c4cc5c(cc4F)C(=O)NCC5)c(N)nc3F)cc2CN(C)C)C[C@H](C)O1]

Prepared from 6-(2-amino-5-bromo-6-fluoropyridin-3-yl)-7-fluoro-3,4-dihydroisoquinolin-1(2H)-one (40 mg, 0.113 mmol) and 1-(2-((2S,6R)-2,6-dimethylmorpholino)-5-(4,4,5,5-tetramethyl-1,3,2-dioxaborolan-2-yl)phenyl)-N,N-dimethylmethanamine hydrochloride (46.4 mg, 0.113 mmol) to give the product (22 mg, 37.1% yield). ¹H NMR (500 MHz, DMSO-d₆) δ=8.12 (br. s., 1H), 7.62 (d, J=9.90 Hz, 2H), 7.53 (br. s., 1H), 7.43 (d, J=6.85 Hz, 2H), 7.15 (br. s., 1H), 6.28 (br. s., 2H), 3.75-3.85 (m, 2H), 3.30-3.55 (m, 4H), 3.09 (br. s., 2H), 2.92 (t, J=6.30 Hz, 2H), 2.12-2.45 (m, 8H), 1.11 (d, J=6.24 Hz, 6H); LCMS: [M+H]⁺=522.55.

### Example 545: (S)-6-(2-amino-5-(3-(azetidin-1-ylmethyl)-4-(2-methylmorpholino)phenyl)-6-fluoropyridin-3-yl)-3,4-dihydroisoquinolin-1(2H)-one (I-598)

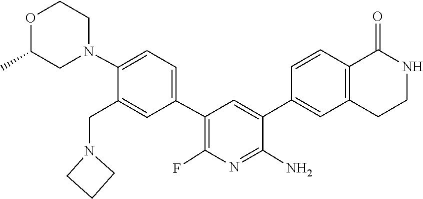 [SMILES: C[C@H]1CN(c2ccc(-c3cc(-c4ccc5c(c4)CCNC5=O)c(N)nc3F)cc2CN2CCC2)CCO1]

Prepared from (S)-4-(2-(azetidin-1-ylmethyl)-4-bromophenyl)-2-methylmorpholine (50 mg, 0.138 mmol, in turn made from 5-bromo-2-fluorobenzaldehyde and (S)-2-methyl-morpholine through an analogous sequence as earlier examples) and 6-(2-amino-6-fluoro-5-(4,4,5,5-tetramethyl-1,3,2-dioxaborolan-2-yl)pyridin-3-yl)-3,4-dihydroisoquinolin-1(2H)-one (68.9 mg, 0.180 mmol) to yield the title compound as an off white solid (35 mg, 48% yield). ¹H NMR (500 MHz, DMSO-d6) δ ppm 7.95 (br. s., 1H), 7.91 (d, J=7.95 Hz, 1H), 7.58 (d, J=10.03 Hz, 1H), 7.44-7.50 (m, 3H), 7.39 (d, J=8.07 Hz, 1H), 7.09 (d, J=8.31 Hz, 1H), 6.25 (s, 2H), 3.86 (d, J=10.88 Hz, 1H), 3.68-3.75 (m, 2H), 3.62 (br. s., 2H), 3.41 (br. s., 2H), 3.12-3.29 (m, 4H), 3.09 (d, J=11.37 Hz, 1H), 3.02 (d, J=11.37 Hz, 1H), 2.96 (t, J=6.48 Hz, 2H), 2.68-2.75 (m, 1H), 2.43 (t, J=10.64 Hz, 1H), 1.99 (t, J=6.72 Hz, 2H), 1.12 (d, J=6.11 Hz, 3H); LCMS: [M+H]⁺=502.43.

### Example 546: (S)-6-(2-amino-5-(3-(azetidin-1-ylmethyl)-4-(2-methylmorpholino)phenyl)-6-fluoropyridin-3-yl)-7-fluoro-3,4-dihydroisoquinolin-1(2H)-one (I-599)

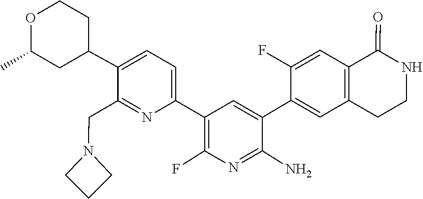 [SMILES: C[C@H]1CC(c2ccc(-c3cc(-c4cc5c(cc4F)C(=O)NCC5)c(N)nc3F)nc2CN2CCC2)CCO1]

Prepared from (S)-4-(2-(azetidin-1-ylmethyl)-4-bromophenyl)-2-methylmorpholine (50 mg, 0.138 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (72.2 mg, 0.180 mmol) to yield the title compound as an off white solid (20 mg, 26% yield). ¹H NMR (500 MHz, DMSO-d₆) δ ppm 8.11 (br. s., 1H), 7.60 (dd, J=17.42, 10.09 Hz, 2H), 7.47 (s, 1 H), 7.43 (d, J=6.97 Hz, 1H), 7.36 (d, J=8.19 Hz, 1H), 7.08 (d, J=8.31 Hz, 1H), 6.26 (s, 2H), 3.86 (d, J=10.76 Hz, 1H), 3.68-3.75 (m, 2H), 3.61 (br. s., 2H), 3.41 (br. s., 2H), 3.12-3.26 (m, 4H), 3.09 (d, J=11.37 Hz, 1H), 3.02 (d, J=11.49 Hz, 1H), 2.93 (t, J=6.36 Hz, 2H), 2.67-2.75 (m, 1H), 2.43 (t, J=10.64 Hz, 1H), 1.99 (quin, J=6.69 Hz, 2H), 1.12 (d, J=6.24 Hz, 3 H); LCMS: [M+H]⁺=520.54.

### Example 547: 6-(6′-amino-6-((dimethylamino)methyl)-2′-fluoro-5-(tetrahydro-2H-pyran-4-yl)-[2,3′-bipyridin]-5′-yl)-3,4-dihydroisoquinolin-1(2H)-one (I-600)

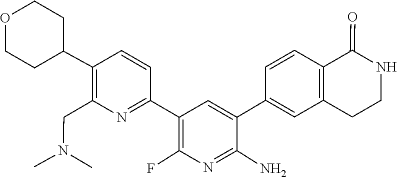 [SMILES: CN(C)Cc1nc(-c2cc(-c3ccc4c(c3)CCNC4=O)c(N)nc2F)ccc1C1CCOCC1]

Step 1: Methyl 6-amino-3-(tetrahydro-2H-pyran-4-yl)picolinate

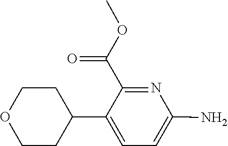 [SMILES: COC(=O)c1nc(N)ccc1C1CCOCC1]

In an autoclave, 10% Pd/C (50% moist, w/w, 5 g) and methyl 6-amino-3-(3,6-dihydro-2H-pyran-4-yl)picolinate (5 g, 21.34 mmol) were added in EtOAc (50 mL). The reaction mass was stirred at 80° C. with 20 Kg/cm³ pressure of H₂ for 36 h. After completion of reaction, reaction mass was filtered through celite bed and the filtrate was concentrated under vacuum to afford product (4.2 g, 83.3% yield) as brown solid used in next step without further purification. LCMS: [M+H]⁺=237.2.

Step 2: Methyl 6-bromo-3-(tetrahydro-2H-pyran-4-yl)picolinate

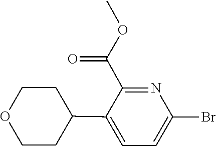 [SMILES: COC(=O)c1nc(Br)ccc1C1CCOCC1]

To a cooled solution of methyl 6-amino-3-(tetrahydro-2H-pyran-4-yl) picolinate (5 g, 21.18 mmol) in 47% HBr in water (35 mL) at 0° C. Br2 (20.3 g, 126.97 mmol) was added dropwise and the reaction mass was stirred at same temperature for 10 minutes. To this, NaNO₂ (8 g, 116.39 mmol) in water (20 mL) was added dropwise at the same temperature and the reaction mass was slowly allowed to warm at room temperature and stirred for 72 h. After completion of reaction, the reaction mass was diluted with water (200 mL) and extracted with EtOAc (3×80 mL). The combined organics was washed with sodium thiosulphate solution (300 mL), dried over Na₂SO₄, concentrate under vacuum to afford crude. The crude was triturated with diethyl ether to afford the product (4 g, 63% yield) as a yellow solid. LCMS: [M+2]⁺=302.

Step 3: (6-bromo-3-(tetrahydro-2H-pyran-4-yl)pyridin-2-yl)methanol

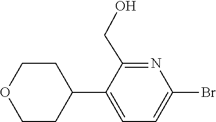 [SMILES: OCc1nc(Br)ccc1C1CCOCC1]

To a cooled solution of methyl 6-bromo-3-(tetrahydro-2H-pyran-4-yl)picolinate (5.5 g, 18.32 mmol) in dry THF (55 mL) at 0° C., LAH (1 M in THF, 18.3 mL, 18.32 mmol) was added dropwise at 0° C. The reaction mass was stirred at RT for 3 h. After completion of reaction, reaction mass was quenched with the very dil. HCl (50 mL) and extracted with ethyl acetate (3×60 mL). The combined organic was dried over anhydrous sodium sulfate, concentrate under vacuum to afford the product (4.5 g, 90.2%) as pale-yellow liquid. LCMS: [M+2]+=274.1.

Step 4: 6-bromo-3-(tetrahydro-2H-pyran-4-yl)picolinaldehyde

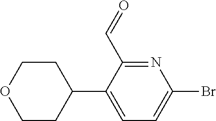 [SMILES: O=Cc1nc(Br)ccc1C1CCOCC1]

To a cooled solution of (6-bromo-3-(tetrahydro-2H-pyran-4-yl)pyridin-2-yl)methanol (4.4 g, 16.16 mmol) in DCM (45 mL) and Dess Martin periodinone (10.3 g, 24.25 mmol) was added at 0° C. portioned wise and the reaction mass was slowly warmed to room temperature and stirred for 16 h. The mixture was diluted with water (150 mL) and extracted with DCM (3×80 mL). The combined organic phase was dried over Na₂SO₄ and concentrated under vacuum to afford the crude was which purified by column chromatography eluting with 15% EtOAc in hexanes and triturating with hexanes to afford the product (2.3 g, 52.7%) as light yellow. LCMS: [M+2]⁺=272.

Step 5: 1-(6-bromo-3-(tetrahydro-2H-pyran-4-yl)pyridin-2-yl)-N,N-dimethylmethanamine

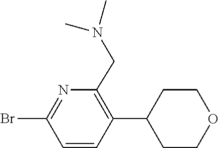 [SMILES: CN(C)Cc1nc(Br)ccc1C1CCOCC1]

Prepared from 6-bromo-3-(tetrahydro-2H-pyran-4-yl)picolinaldehyde (135 mg, 0.500 mmol) and dimethylamine, 2.0M solution in THF (1 mL, 1.999 mmol) in a manner similar to previous examples. The title compound was isolated as an off white solid (149 mg, 95% yield, 95% purity). LCMS: [M+H]⁺=299.34.

Step 6: 6-(6′-amino-6-((dimethylamino)methyl)-2′-fluoro-5-(tetrahydro-2H-pyran-4-yl)-[2,3′-bipyridin]-5′-yl)-3,4-dihydroisoquinolin-1(2H)-one

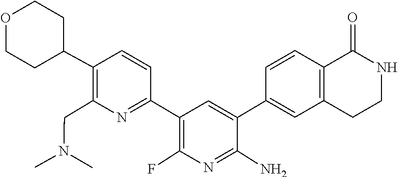 [SMILES: CN(C)Cc1nc(-c2cc(-c3ccc4c(c3)CCNC4=O)c(N)nc2F)ccc1C1CCOCC1]

Prepared from 1-(6-bromo-3-(tetrahydro-2H-pyran-4-yl)pyridin-2-yl)-N,N-dimethylmethanamine (70 mg, 0.222 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (111 mg, 0.289 mmol) to yield the title compound as a white solid (91.5 mg, 82% yield). ¹H NMR (500 MHz, DMSO-d₆) δ ppm 8.09 (d, J=9.90 Hz, 1H), 7.96 (br. s., 1H), 7.93 (d, J=7.95 Hz, 1H), 7.78 (d, J=8.19 Hz, 1H), 7.64 (d, J=7.70 Hz, 1H), 7.38-7.45 (m, 2H), 6.46 (br. s., 2H), 3.97 (d, J=10.27 Hz, 2H), 3.59 (s, 2H), 3.40-3.47 (m, 4H), 3.20-3.28 (m, 1H), 2.96 (t, J=6.42 Hz, 2H), 2.16 (s, 6H), 1.63-1.75 (m, 4H); LCMS: [M+H]⁺=476.42.

### Example 548: 6-(6′-amino-6-((dimethylamino)methyl)-2′-fluoro-5-(tetrahydro-2H-pyran-4-yl)-[2,3′-bipyridin]-5′-yl)-7-fluoro-3,4-dihydroisoquinolin-1(2H)-one (I-601)

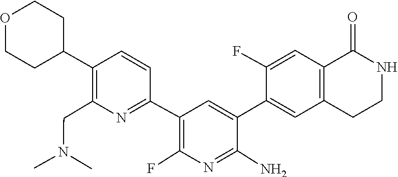 [SMILES: CN(C)Cc1nc(-c2cc(-c3cc4c(cc3F)C(=O)NCC4)c(N)nc2F)ccc1C1CCOCC1]

Prepared from 1-(6-bromo-3-(tetrahydro-2H-pyran-4-yl)pyridin-2-yl)-N,N-dimethylmethanamine (70 mg, 0.222 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (116 mg, 0.289 mmol) to yield the title compound as a white solid (55 mg, 48% yield). ¹H NMR (500 MHz, DMSO-d₆) δ ppm 8.12 (br. s., 1H), 8.08 (d, J=9.90 Hz, 1H), 7.78 (d, J=8.19 Hz, 1H), 7.58-7.67 (m, 2H), 7.41 (d, J=6.97 Hz, 1H), 6.49 (br. s., 2H), 3.97 (d, J=10.15 Hz, 2H), 3.58 (s, 2H), 3.40-3.48 (m, 4H), 3.25 (t, J=11.31 Hz, 1H), 2.93 (t, J=6.24 Hz, 2H), 2.15, (s, 6H), 1.61-1.74 (m, 4H); LCMS: [M+H]⁺=494.47.

### Example 549: 6-(6′-amino-6-(azetidin-1-ylmethyl)-2′-fluoro-5-(tetrahydro-2H-pyran-4-yl)-[2,3′-bipyridin]-5′-yl)-7-fluoro-3,4-dihydroisoquinolin-1(2H)-one (I-602)

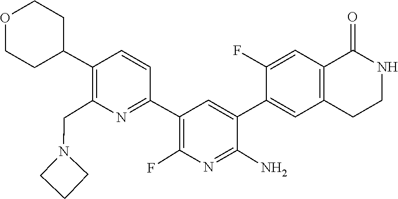 [SMILES: Nc1nc(F)c(-c2ccc(C3CCOCC3)c(CN3CCC3)n2)cc1-c1cc2c(cc1F)C(=O)NCC2]

Prepared from 2-(azetidin-1-ylmethyl)-6-bromo-3-(tetrahydro-2H-pyran-4-yl)pyridine (80 mg, 0.231 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (134 mg, 0.334 mmol) to yield the title compound as an off white solid (62 mg, 45% yield). ¹H NMR (500 MHz, DMSO-d₆) δ ppm 8.07-8.17 (m, 2H), 7.74 (d, J=8.31 Hz, 1H), 7.63 (t, J=9.96 Hz, 2 H), 7.42 (d, J=6.97 Hz, 1H), 6.51 (br. s., 2H), 3.97 (d, J=10.51 Hz, 2H), 3.78 (br. s., 2H), 3.46-3.51 (m, 2H), 3.44 (br. s., 2H), 3.09-3.24 (m, 5H), 2.94 (t, J=6.36 Hz, 2H), 1.90-2.00 (m, 2H), 1.64-1.74 (m, 4H); LCMS: [M+H]⁺=506.44.

### Example 550: 6-(6′-amino-6-((ethyl(methyl)amino)methyl)-2′-fluoro-5-(tetrahydro-2H-pyran-4-yl)-[2,3′-bipyridin]-5′-yl)-3,4-dihydroisoquinolin-1(2H)-one (I-603)

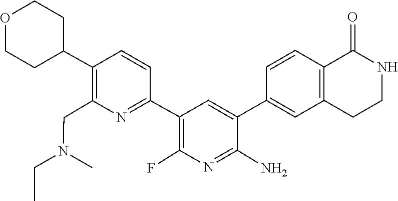 [SMILES: CCN(C)Cc1nc(-c2cc(-c3ccc4c(c3)CCNC4=O)c(N)nc2F)ccc1C1CCOCC1]

Prepared from N-((6-bromo-3-(tetrahydro-2H-pyran-4-yl)pyridin-2-yl)methyl)-N-methylethanamine (70 mg, 0.201 mmol, in turn made from 6-bromo-3-(tetrahydro-2H-pyran-4-yl)picolinaldehyde and N-ethylmethylamine in a manner similar to previous examples) and 6-(2-amino-6-fluoro-5-(4,4,5,5-tetramethyl-1,3,2-dioxaborolan-2-yl)pyridin-3-yl)-3,4-dihydroisoquinolin-1(2H)-one (100 mg, 0.261 mmol) to yield the title compound as a white solid (91 mg, 88% yield). ¹H NMR (500 MHz, DMSO-d₆) δ ppm 8.09 (d, J=9.90 Hz, 1 H), 7.96 (br. s., 1H), 7.93 (d, J=7.82 Hz, 1H), 7.77 (d, J=8.19 Hz, 1H), 7.64 (d, J=7.70 Hz, 1H), 7.37-7.45 (m, 2H), 6.47 (br. s., 2H), 3.98 (d, J=10.15 Hz, 2H), 3.66 (s, 2H), 3.40-3.47 (m, 4H), 3.28 (br. s., 1H), 2.96 (t, J=6.17 Hz, 2H), 2.42 (q, J=6.93 Hz, 2H), 2.08 (s, 3 H), 1.63-1.76 (m, 4H), 1.03 (t, J=6.97 Hz, 3H); LCMS: [M+H]⁺=490.52.

### Example 551: 6-(6′-amino-6-((ethyl(methyl)amino)methyl)-2′-fluoro-5-(tetrahydro-2H-pyran-4-yl)-[2,3′-bipyridin]-5′-yl)-7-fluoro-3,4-dihydroisoquinolin-1(2H)-one (I-604)

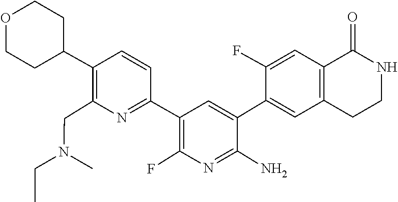 [SMILES: CCN(C)Cc1nc(-c2cc(-c3cc4c(cc3F)C(=O)NCC4)c(N)nc2F)ccc1C1CCOCC1]

Prepared from N-((6-bromo-3-(tetrahydro-2H-pyran-4-yl)pyridin-2-yl)methyl)-N-methylethanamine (70 mg, 0.201 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (82 mg, 0.205 mmol) to yield the title compound as an off white solid (55 mg, 65% yield). ¹H NMR (500 MHz, DMSO-d₆) δ ppm 8.12 (br. s., 1H), 8.09 (d, J=9.90 Hz, 1H), 7.77 (d, J=8.19 Hz, 1H), 7.60-7.68 (m, 2H), 7.41 (d, J=6.97 Hz, 1H), 6.50 (br. s., 2H), 3.98 (d, J=10.15 Hz, 2 H), 3.65 (s, 2H), 3.38-3.47 (m, 4H), 3.25-3.31 (m, 1H), 2.93 (t, J=6.30 Hz, 2H), 2.41 (q, J=6.72 Hz, 2H), 2.07 (s, 3H), 1.63-1.76 (m, 4H), 1.02 (t, J=7.03 Hz, 3H); LCMS: [M+H]⁺=508.51.

### Example 552: 6-(6′-amino-6-(azetidin-1-ylmethyl)-2′-fluoro-5-(tetrahydro-2H-pyran-4-yl)-[2,3′-bipyridin]-5′-yl)-3,4-dihydroisoquinolin-1(2H)-one (I-605)

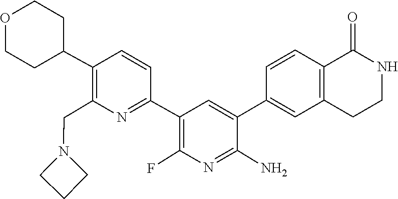 [SMILES: Nc1nc(F)c(-c2ccc(C3CCOCC3)c(CN3CCC3)n2)cc1-c1ccc2c(c1)CCNC2=O]

Prepared from 2-(azetidin-1-ylmethyl)-6-bromo-3-(tetrahydro-2H-pyran-4-yl)pyridine (80 mg, 0.231 mmol, in turn made from 6-bromo-3-(tetrahydro-2H-pyran-4-yl)picolinaldehyde and azetidine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (115 mg, 0.301 mmol) to yield the title compound as a beige solid (41 mg, 35% yield). ¹H NMR (500 MHz, DMSO-d₆) b 8.1-8.2 (m, 1H), 8.0-8.0 (m, 1H), 7.9-8.0 (m, 1H), 7.7-7.8 (m, 1H), 7.6-7.7 (m, 1H), 7.4-7.5 (m, 2H), 6.3-6.6 (m, 2H), 3.9-4.0 (m, 2H), 3.7-3.8 (m, 2H), 3.4-3.5 (m, 4H), 3.1-3.2 (m, 5H), 2.97 (br t, 2H, J=6.4 Hz), 1.9-2.0 (m, 2H), 1.6-1.8 (m, 4H); LCMS: [M+H]⁺=488.45.

### Example 553: 6-(2-amino-5-(3-((dimethylamino)methyl)-2-fluoro-4-morpholinophenyl)-6-fluoropyridin-3-yl)-3,4-dihydroisoquinolin-1(2H)-one (I-606)

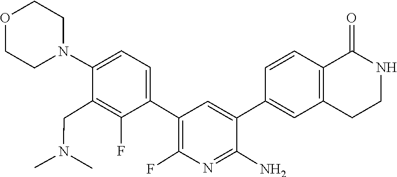 [SMILES: CN(C)Cc1c(N2CCOCC2)ccc(-c2cc(-c3ccc4c(c3)CCNC4=O)c(N)nc2F)c1F]

Prepared from 1-(3-bromo-2-fluoro-6-morpholinophenyl)-N,N-dimethylmethanamine (60 mg, 0.180 mmol, in turn made using a synthetic sequence starting from 2,6-difluorobenzaldehyde and morpholine analogous to previous examples) and 6-(2-amino-6-fluoro-5-(4,4,5,5-tetramethyl-1,3,2-dioxaborolan-2-yl)pyridin-3-yl)-3,4-dihydroisoquinolin-1(2H)-one (90 mg, 0.234 mmol) to yield the title compound as a beige solid (87 mg, 93% yield). ¹H NMR (500 MHz, DMSO-d₆) δ=8.00-7.92 (m, 1H), 7.92-7.87 (m, 1H), 7.54-7.49 (m, 1H), 7.48-7.42 (m, 2H), 7.42-7.34 (m, 1H), 7.07-6.94 (m, 1H), 6.35 (br s, 2H), 3.83-3.73 (m, 4H), 3.58-3.44 (m, 2H), 3.44-3.39 (m, 2H), 3.14-3.03 (m, 4H), 2.97-2.90 (m, 2H), 2.34-2.08 (m, 6H); LCMS: [M+H]⁺=494.37.

### Example 554: 6-(2-amino-5-(3-((dimethylamino)methyl)-2-fluoro-4-morpholinophenyl)-6-fluoropyridin-3-yl)-7-fluoro-3,4-dihydroisoquinolin-1(2H)-one (I-607)

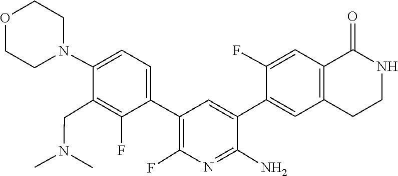 [SMILES: CN(C)Cc1c(N2CCOCC2)ccc(-c2cc(-c3cc4c(cc3F)C(=O)NCC4)c(N)nc2F)c1F]

Prepared from 1-(3-bromo-2-fluoro-6-morpholinophenyl)-N,N-dimethylmethanamine (60 mg, 0.180 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (94 mg, 0.234 mmol) yield the title compound as a beige solid (42 mg, 43% yield). ¹H NMR (500 MHz, DMSO-d₆) δ=8.19-8.04 (m, 1H), 7.61 (d, J=10.1 Hz, 1H), 7.53-7.47 (m, 1H), 7.43-7.38 (m, 1H), 7.37-7.31 (m, 1H), 7.03-6.97 (m, 1H), 6.35 (br s, 2H), 3.76 (br d, J=4.0 Hz, 4H), 3.51-3.46 (m, 2H), 3.40 (br dd, J=4.0, 6.1 Hz, 2H), 3.09 (br s, 4H), 2.92 (br t, J=6.3 Hz, 2H), 2.25-2.18 (m, 6H); LCMS: [M+H]⁺=512.39.

### Example 555: 6-(2-amino-5-(3-(azetidin-1-ylmethyl)-2-fluoro-4-morpholinophenyl)-6-fluoropyridin-3-yl)-7-fluoro-3,4-dihydroisoquinolin-1(2H)-one(I-608)

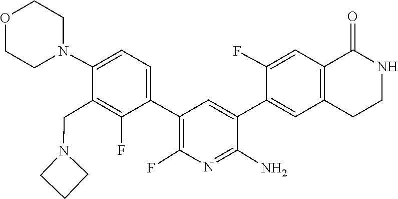 [SMILES: Nc1nc(F)c(-c2ccc(N3CCOCC3)c(CN3CCC3)c2F)cc1-c1cc2c(cc1F)C(=O)NCC2]

Prepared from 4-(2-(azetidin-1-ylmethyl)-4-bromo-3-fluorophenyl)morpholine (60 mg, 0.173 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (90 mg, 0.225 mmol) to yield the title compound as a beige solid (45 mg, 47% yield). 1H NMR (500 MHz, DMSO-d₆) δ ppm =8.16-8.08 (m, 1H), 7.61 (d, J=10.0 Hz, 1H), 7.52-7.46 (m, 1H), 7.41 (d, J=7.0 Hz, 1H), 7.34-7.25 (m, 1H), 6.97-6.92 (m, 1H), 6.43-6.29 (m, 2H), 3.79-3.72 (m, 4H), 3.58 (br s, 2H), 3.44-3.38 (m, 2H), 3.24-3.18 (m, 4H), 3.06 (br s, 4H), 2.92 (br t, J=6.4 Hz, 2H), 1.93-1.86 (m, 2H); LCMS: [M+H]⁺=524.43.

### Example 556: 6-(2-amino-5-(3-(azetidin-1-ylmethyl)-2-fluoro-4-morpholinophenyl)-6-fluoropyridin-3-yl)-3,4-dihydroisoquinolin-1(2H)-one (I-609)

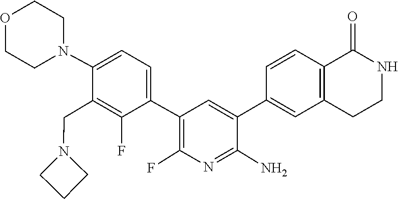 [SMILES: Nc1nc(F)c(-c2ccc(N3CCOCC3)c(CN3CCC3)c2F)cc1-c1ccc2c(c1)CCNC2=O]

Prepared from 4-(2-(azetidin-1-ylmethyl)-4-bromo-3-fluorophenyl)morpholine (60 mg, 0.173 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (86 mg, 0.225 mmol) to yield the title compound as a white solid (30 mg, 33% yield). ¹H NMR (500 MHz, DMSO-d₆) δ ppm 7.94 (br s, 1H), 7.9-7.9 (m, 1H), 7.50 (br d, 1H, J=9.5 Hz), 7.4-7.5 (m, 2H), 7.3-7.4 (m, 1H), 6.9-7.0 (m, 1H), 6.34 (br s, 2H), 3.75 (br s, 4H), 3.59 (br s, 2H), 3.40 (br s, 2H), 3.21 (br t, 4H, J=6.5 Hz), 3.06 (br s, 4H), 2.95 (br t, 2H, J=6.2 Hz), 1.90 (quin, 2H, J=6.5 Hz); LCMS: [M+H]+=506.38.

### Example 557: 6-(2-amino-5-(3-((ethyl(methyl)amino)methyl)-2-fluoro-4-morpholinophenyl)-6-fluoropyridin-3-yl)-3,4-dihydroisoquinolin-1(2H)-one (I-610)

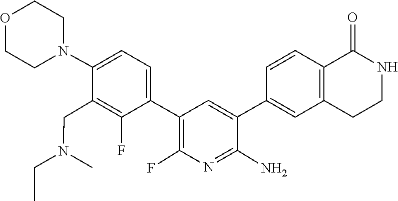 [SMILES: CCN(C)Cc1c(N2CCOCC2)ccc(-c2cc(-c3ccc4c(c3)CCNC4=O)c(N)nc2F)c1F]

Prepared from N-(3-bromo-2-fluoro-6-morpholinobenzyl)-N-methylethanamine (70 mg, 0.173 mmol, in turn made from 3-bromo-2-fluoro-6-morpholinobenzaldehyde and N-ethylmethylamine in a manner analogous to previous examples) and 6-(2-amino-6-fluoro-5-(4,4,5,5-tetramethyl-1,3,2-dioxaborolan-2-yl)pyridin-3-yl)-3,4-dihydroisoquinolin-1(2H)-one (86 mg, 0.225 mmol) to yield the title compound as a white solid (27 mg, 29% yield). ¹H NMR (500 MHz, DMSO-d₆) δ ppm 7.9-8.0 (m, 1H), 7.8-7.9 (m, 1H), 7.5-7.5 (m, 1H), 7.4-7.5 (m, 2H), 7.3-7.4 (m, 1H), 7.0-7.0 (m, 1H), 6.3-6.4 (m, 2H), 3.75 (br s, 4H), 3.54 (br s, 2H), 3.40 (br s, 2H), 3.09 (br s, 4H), 2.95 (br t, 2H, J=6.2 Hz), 2.4-2.5 (m, 2H), 2.15 (s, 3H), 1.0-1.1 (m, 3H); LCMS: [M+H]⁺=508.38.

### Example 558: 6-(2-amino-5-(3-((ethyl(methyl)amino)methyl)-2-fluoro-4-morpholinophenyl)-6-fluoropyridin-3-yl)-7-fluoro-3,4-dihydroisoquinolin-1(2H)-one (I-611)

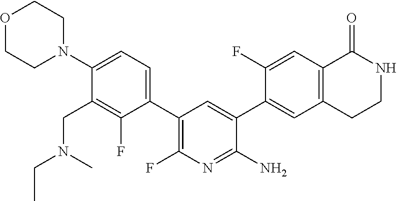 [SMILES: CCN(C)Cc1c(N2CCOCC2)ccc(-c2cc(-c3cc4c(cc3F)C(=O)NCC4)c(N)nc2F)c1F]

Prepared from N-(3-bromo-2-fluoro-6-morpholinobenzyl)-N-methylethanamine (70 mg, 0.173 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (90 mg, 0.225 mmol) to yield the title compound as an off white solid (58 mg, 61% yield). ¹H NMR (500 MHz, DMSO-d₆) δ 8.1-8.2 (m, 1H), 7.6-7.6 (m, 1H), 7.5-7.5 (m, 1H), 7.42 (br d, 1H, J=6.8 Hz), 7.3-7.4 (m, 1H), 6.9-7.1 (m, 1H), 6.36 (br s, 2H), 3.75 (br s, 4H), 3.54 (br s, 2H), 3.4-3.4 (m, 2H), 3.0-3.2 (m, 4H), 2.91 (br t, 2H, J=6.0 Hz), 2.47 (br d, 2H, J=6.4 Hz), 2.0-2.2 (m, 3H), 0.9-1.1 (m, 3H); LCMS: [M+H]⁺=526.50.

### Example 559: 6-(6-amino-5′-((dimethylamino)methyl)-2-fluoro-6′-morpholino-[3,3′-bipyridin]-5-yl)-3,4-dihydroisoquinolin-1(2H)-one (I-612)

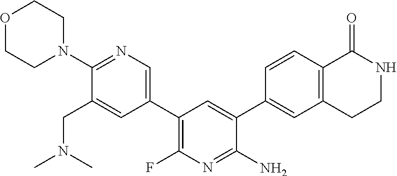 [SMILES: CN(C)Cc1cc(-c2cc(-c3ccc4c(c3)CCNC4=O)c(N)nc2F)cnc1N1CCOCC1]

Step 1: 5-bromo-2-morpholinonicotinaldehyde

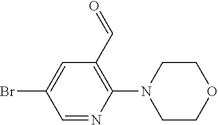 [SMILES: O=Cc1cc(Br)cnc1N1CCOCC1]

To a solution of 5-bromo-2-fluoropyridine-3-carboxaldehyde (1.0 g, 4.90 mmol) and K₂CO₃ (1.355 g, 9.80 mmol) in N,N-dimethylformamide (5 ml) was added morpholine (0.444 ml, 5.15 mmol). The mixture was stirred at 80° C. for 2 h then partitioned between water (100 mL) and EtOAc (20 mL). The organic phase was separated, and the aqueous phase was washed with EtOAc (20 mL). The combined organic phases were washed with water, followed by brine, dried over Na₂SO₄, and concentrated. The crude was purified by flash sgc eluting with 0-30% EtOAc/Hexanes. The desired fractions were collected, concentrated and dried under vacuum to afford the product (895 mg, 67% yield) as a yellow solid. LCMS [M+H]⁺=271.10.

Step 2: 1-(5-bromo-2-morpholinopyridin-3-yl)-N,N-dimethylmethanamine

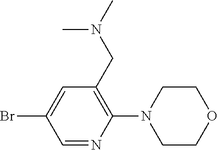 [SMILES: CN(C)Cc1cc(Br)cnc1N1CCOCC1]

Prepared from 5-bromo-2-morpholinonicotinaldehyde (250 mg, 0.922 mmol) and dimethylamine, 2.0M solution in THF (1.383 mL, 2.77 mmol) to give the product (266 mg, 0.886 mmol, 96% yield) as an off-white solid. LCMS: [M+H]⁺=300.28.

Step 3: (5-((dimethylamino)methyl)-6-morpholinopyridin-3-yl)boronic acid

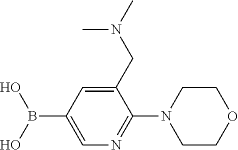 [SMILES: CN(C)Cc1cc(B(O)O)cnc1N1CCOCC1]

Prepared from 1-(5-bromo-2-morpholinopyridin-3-yl)-N,N-dimethylmethanamine (150 mg, 0.500 mmol) to give the boronic acid which was used in the next step without further purification. LCMS [M+H]⁺=266.37.

Step 4: 6-(2-amino-5-(3-((dimethylamino)methyl)-4-morpholinophenyl)-6-fluoropyridin-3-yl)-4-methylisoquinolin-1(2H)-one

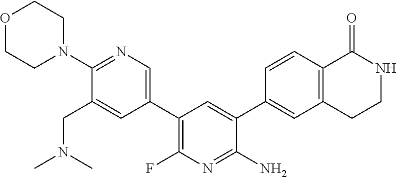 [SMILES: CN(C)Cc1cc(-c2cc(-c3ccc4c(c3)CCNC4=O)c(N)nc2F)cnc1N1CCOCC1]

Prepared from 6-(2-amino-5-bromo-6-fluoropyridin-3-yl)-3,4-dihydroisoquinolin-1(2H)-one (40 mg, 0.119 mmol) and (5-((dimethylamino)methyl)-6-morpholinopyridin-3-yl)boronic acid (37.9 mg, 0.143 mmol) to give the title compound (29.7 mg, 52.4% yield) as a light grey solid. ¹H NMR (500 MHz, DMSO-d₆) δ ppm 8.36 (s, 1H), 7.94 (br. s., 1H), 7.91 (d, J=7.83 Hz, 1H), 7.85 (s, 1H), 7.67 (d, J=10.03 Hz, 1H), 7.47 (d, J=8.19 Hz, 1H), 7.45 (s, 1H), 6.33 (br. s., 2H), 3.71-3.78 (m, 4H), 3.40 (s, 4H), 3.14-3.20 (m, 4H), 2.95 (t, J=6.42 Hz, 2H), 2.19 (s, 6H); LCMS [M+H]⁺ 477.42.

### Example 560: 6-(6′-amino-4-((dimethylamino)methyl)-2′-fluoro-5-morpholino-[2,3′-bipyridin]-5′-yl)-3,4-dihydroisoquinolin-1(2H)-one (I-613)

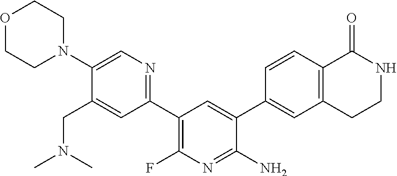 [SMILES: CN(C)Cc1cc(-c2cc(-c3ccc4c(c3)CCNC4=O)c(N)nc2F)ncc1N1CCOCC1]

Prepared from 6-(2-amino-6-fluoro-5-(4,4,5,5-tetramethyl-1,3,2-dioxaborolan-2-yl)pyridin-3-yl)-3,4-dihydroisoquinolin-1(2H)-one (79 mg, 0.206 mmol, prepared from a sequence starting from 2-bromo-5-fluoro-4-formylpyridine and N,N-diisopropylethylamine (1.708 mL, 9.80 mmol) and morpholine) and 1-(2-bromo-5-morpholinopyridin-4-yl)-N,N-dimethylmethanamine (120 mg, 0.172 mmol) to give the title compound (35.7 mg, 43.6% yield) as a white solid. ¹H NMR (500 MHz, DMSO-d₆) δ ppm 8.35 (s, 1H), 8.11 (d, J=10.03 Hz, 1H), 7.94 (br. s., 1H), 7.92 (d, J=7.95 Hz, 1H), 7.81 (s, 1H), 7.43 (d, J=8.07 Hz, 1H), 7.41 (s, 1H), 6.47 (br. s., 2H), 3.74 (d, J=4.03 Hz, 4H), 3.48 (s, 2H), 3.39-3.45 (m, 2H), 2.94-3.02 (m, 6H), 2.21 (s, 6H); LCMS: [M+H]⁺=477.42.

### Example 561: 6-(2-amino-6-fluoro-5-(3-((isopropyl(methyl)amino)methyl)-4-morpholinophenyl)pyridin-3-yl)-3,4-dihydroisoquinolin-1(2H)-one (I-614)

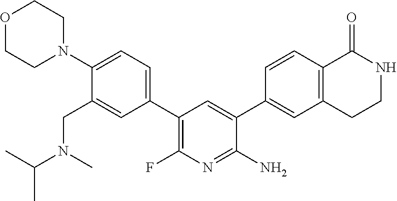 [SMILES: CC(C)N(C)Cc1cc(-c2cc(-c3ccc4c(c3)CCNC4=O)c(N)nc2F)ccc1N1CCOCC1]

Prepared from N-(5-bromo-2-morpholinobenzyl)-N-methylpropan-2-amine (40 mg, 0.122 mmol, in turn prepared using a sequence starting from 2-bromo-5-fluoro-4-formylpyridine and morpholine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (60.9 mg, 0.159 mmol) to yield the title compound as a white solid (23 mg, 36% yield). ¹H NMR (500 MHz, DMSO-d₆) δ 8.0-8.1 (m, 1H), 7.9-8.0 (m, 1H), 7.6-7.7 (m, 2H), 7.5-7.5 (m, 2H), 7.45 (br d, 1H, J=7.8 Hz), 7.19 (br d, 1H, J=8.2 Hz), 6.31 (br s, 2H), 3.8-3.8 (m, 4H), 3.63 (br s, 2H), 3.5-3.5 (m, 2H), 3.0-3.0 (m, 6H), 2.9-2.9 (m, 1H), 2.1-2.2 (m, 3H), 1.0-1.1 (m, 6H); LCMS: [M+H]⁺=504.44.

### Example 562: 6-(2-amino-5-(3-((diethylamino)methyl)-4-morpholinophenyl)-6-fluoropyridin-3-yl)-3,4-dihydroisoquinolin-1(2H)-one (I-615)

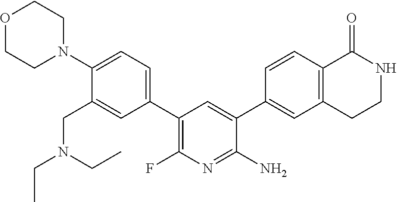 [SMILES: CCN(CC)Cc1cc(-c2cc(-c3ccc4c(c3)CCNC4=O)c(N)nc2F)ccc1N1CCOCC1]

Prepared from N-(5-bromo-2-morpholinobenzyl)-N-ethylethanamine (45 mg, 0.138 mmol, prepared from a sequence starting from 5-bromo-2-morpholinobenzaldehyde and diethylamine, analogous to previous examples) and 6-(2-amino-6-fluoro-5-(4,4,5,5-tetramethyl-1,3,2-dioxaborolan-2-yl)pyridin-3-yl)-3,4-dihydroisoquinolin-1(2H)-one (68.5 mg, 0.179 mmol) to yield the title compound as a beige solid (37 mg, 51% yield). ¹H NMR (500 MHz, DMSO-d₆) δ 7.9-8.0 (m, 1H), 7.9-7.9 (m, 1H), 7.63 (br s, 1H), 7.58 (br d, 1H, J=10.0 Hz), 7.4-7.5 (m, 2H), 7.3-7.4 (m, 1H), 7.1-7.2 (m, 1H), 6.2-6.3 (m, 2H), 3.73 (br s, 4H), 3.60 (br s, 2H), 3.52 (br s, 2H), 2.9-3.0 (m, 2H), 2.89 (br s, 4H), 2.4-2.5 (m, 4H), 0.9-1.0 (m, 6H); LCMS: [M+H]⁺=504.44.

### Example 563: 6-(2-amino-6-fluoro-5-(3-((methyl((tetrahydrofuran-2-yl)methyl)amino)methyl)-4-morpholinophenyl)pyridin-3-yl)-3,4-dihydroisoquinolin-1(2H)-one (I-616)

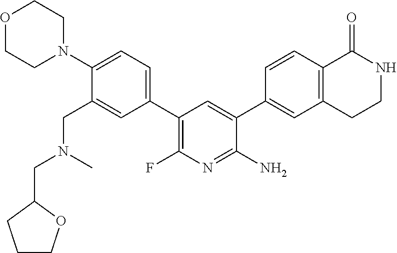 [SMILES: CN(Cc1cc(-c2cc(-c3ccc4c(c3)CCNC4=O)c(N)nc2F)ccc1N1CCOCC1)CC1CCCO1]

Prepared from N-(5-bromo-2-morpholinobenzyl)-N-methyl-1-(tetrahydrofuran-2-yl)methanamine (35 mg, 0.095 mmol, prepared from a sequence starting from 5-bromo-2-morpholinobenzaldehyde and methyl(oxaolan-2-ylmethyl)amine) and 6-(2-amino-6-fluoro-5-(4,4,5,5-tetramethyl-1,3,2-dioxaborolan-2-yl)pyridin-3-yl)-3,4-dihydroisoquinolin-1(2H)-one (47.2 mg, 0.123 mmol) to yield the title compound as a white solid (22.5 mg, 41% yield). ¹H NMR (500 MHz, DMSO-d₆) δ=7.98-7.93 (m, 1H), 7.91 (d, J=7.9 Hz, 1H), 7.65-7.58 (m, 2H), 7.48-7.45 (m, 1H), 7.44 (s, 1H), 7.43-7.39 (m, 1H), 7.16-7.11 (m, 1H), 6.32-6.19 (m, 2H), 3.98-3.90 (m, 1H), 3.79-3.72 (m, 4H), 3.65 (q, J=7.2 Hz, 1H), 3.62-3.56 (m, 2H), 3.56-3.51 (m, 1H), 3.45-3.38 (m, 2H), 2.99-2.89 (m, 6H), 2.47-2.38 (m, 2H), 2.28-2.20 (m, 3H), 1.93-1.84 (m, 1H), 1.77-1.69 (m, 2H), 1.49-1.39 (m, 1H); LCMS: [M+H]⁺=546.43.

### Example 564: 6-(2-amino-5-(3-((dimethylamino)methyl)-4-fluorophenyl)-6-fluoropyridin-3-yl)-3,4-dihydroisoquinolin-1(2H)-one (I-617)

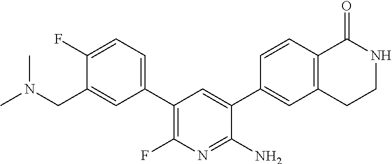 [SMILES: CN(C)Cc1cc(-c2cc(-c3ccc4c(c3)CCNC4=O)c(N)nc2F)ccc1F]

Prepared from 1-(5-bromo-2-fluorophenyl)-N,N-dimethylmethanamine (60 mg, 0.230 mmol, in turn prepared from 5-bromo-2-fluorobenzaldehyde and Dimethylamine, 2.0M solution in THF as per previous procedures) and 6-(2-amino-6-fluoro-5-(4,4,5,5-tetramethyl-1,3,2-dioxaborolan-2-yl)pyridin-3-yl)-3,4-dihydroisoquinolin-1(2H)-one (115 mg, 0.299 mmol) to afford the title compound as a white solid (45 mg, 46% yield). ¹H NMR (500 MHz, DMSO-d₆) δ 7.9-8.0 (m, 1H), 7.9-7.9 (m, 1H), 7.6-7.7 (m, 1H), 7.5-7.6 (m, 1H), 7.4-7.5 (m, 3H), 7.2-7.2 (m, 1H), 6.33 (s, 2H), 3.5-3.5 (m, 2H), 3.4-3.4 (m, 2H), 2.9-3.0 (m, 2H), 2.1-2.2 (m, 6H); LCMS: [M+H]⁺=409.35.

### Example 565: 6-(2-amino-5-(3-((dimethylamino)methyl)-4-fluorophenyl)-6-fluoropyridin-3-yl)-7-fluoro-3,4-dihydroisoquinolin-1(2H)-one (I-618)

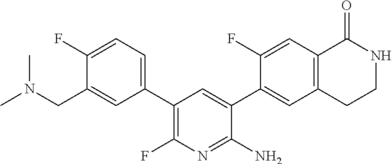 [SMILES: CN(C)Cc1cc(-c2cc(-c3cc4c(cc3F)C(=O)NCC4)c(N)nc2F)ccc1F]

Prepared from 1-(5-bromo-2-fluorophenyl)-N,N-dimethylmethanamine (60 mg, 0.230 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (120 mg, 0.299 mmol) to afford the title compound as a white solid (23.5 mg, 23% yield). ¹H NMR (500 MHz, DMSO-d₆) δ 8.1-8.2 (m, 1H), 7.6-7.7 (m, 2H), 7.5-7.6 (m, 1H), 7.5-7.5 (m, 1H), 7.4-7.5 (m, 1H), 7.2-7.3 (m, 1H), 6.3-6.4 (m, 2H), 3.5-3.5 (m, 2H), 3.4-3.4 (m, 2H), 2.9-3.0 (m, 2H), 2.1-2.2 (m, 6H). LCMS: [M+H]⁺=427.28.

### Example 566: 6-(2-amino-6-fluoro-5-(3-((methyl((tetrahydrofuran-2-yl)methyl)amino)methyl)-4-morpholinophenyl)pyridin-3-yl)-7-fluoro-3,4-dihydroisoquinolin-1(2H)-one (I-619)

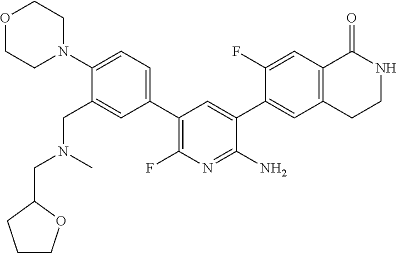 [SMILES: CN(Cc1cc(-c2cc(-c3cc4c(cc3F)C(=O)NCC4)c(N)nc2F)ccc1N1CCOCC1)CC1CCCO1]

Prepared from N-(5-bromo-2-morpholinobenzyl)-N-methyl-1-(tetrahydrofuran-2-yl)methanamine (35 mg, 0.095 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (49.4 mg, 0.123 mmol) to yield the title compound as a white solid (7.6 mg, 13% yield). ¹H NMR (500 MHz, DMSO-d₆) δ 8.0-8.1 (m, 1H), 7.5-7.6 (m, 3H), 7.33 (br dd, 2H, J=7.4, 16.8 Hz), 7.0-7.1 (m, 1H), 6.0-6.3 (m, 2H), 3.8-3.9 (m, 1H), 3.6-3.7 (m, 4H), 3.5-3.6 (m, 1H), 3.4-3.5 (m, 3H), 3.3-3.4 (m, 2H), 2.8-2.9 (m, 6H), 2.35 (br d, 2H, J=6.2 Hz), 2.16 (br s, 3H), 1.7-1.8 (m, 1H), 1.6-1.7 (m, 2H), 1.3-1.4 (m, 1H); LCMS: [M+H]⁺=564.41.

### Example 567: 6-(2-amino-5-(3-((dimethylamino)methyl)-4-(tetrahydrofuran-3-yl)phenyl)-6-fluoropyridin-3-yl)-3,4-dihydroisoquinolin-1(2H)-one (I-620)

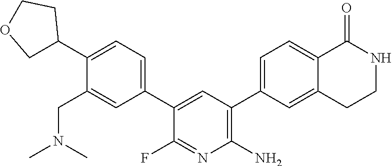 [SMILES: CN(C)Cc1cc(-c2cc(-c3ccc4c(c3)CCNC4=O)c(N)nc2F)ccc1C1CCOC1]

Step 1: 3-(prop-2-yn-1-yloxy)prop-1-ene

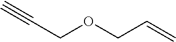

60% NaH in mineral oil (30 g, 750 mmol) was washed with hexanes (3×200 mL) and diethyl ether (100 mL) under nitrogen atmosphere and diethyl ether (500 mL) was added to it; followed by dropwise addition of prop-2-en-1-ol (36 g, 620 mmol) (vigorous gas evolution and reaction mass thickening was observed). After Completion of the addition, the mixture was heated at 30° C. for 1 h and propargyl bromide (178 g, 750 mmol) was added dropwise at RT. The reaction mass was stirred for 16 h at 35-40° C. After 16 h, the mixture was cooled to RT and quenched with water (250 mL) and layers were separated. The organic phase was used as such for the next step without any analysis.

Step 2: 2-(3-(allyloxy)prop-1-en-2-yl)-4,4,5,5-tetramethyl-1,3,2-dioxaborolane

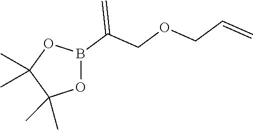 [SMILES: C=CCOCC(=C)B1OC(C)(C)C(C)(C)O1]

To a diethyl ether solution of 3-(prop-2-yn-1-yloxy)prop-1-ene (48 g, 500 mmol) in toluene (1.5 L) was added CuCl (4.95 g, 50 mmol), bis(pinacolato)diboron (139 g, 550 mmol), (t-Bu)3P.HBF4 (17.46 g, 60 mmol) and t-BuONa (14.4 g, 150 mmol) at RT under nitrogen atmosphere. After 5 min, methanol (32 mL, 1000 mmol) was dropwise added at RT and the reaction mass was heated up to 80° C. and allowed to stir at RT for 16 h. The mixture was passed through silica and eluted in hexane to afford the product (15 g, 13.4%) as a light yellow to green liquid. ¹H NMR (400 MHz, CDCl3) δ 5.99-5.92 (t, J=13.6 Hz, 2H), 5.30 (d, J=12.4 Hz, 1H), 5.18 (d, J=14 Hz, 1H), 4.09 (s, 2H), 4.03 (d, J=5.6 Hz, 1H), 1.29 (s, 12H)

Step 3: 2-(2,5-dihydrofuran-3-yl)-4,4,5,5-tetramethyl-1,3,2-dioxaborolane

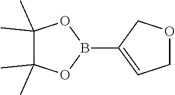

A solution 2-(3-(allyloxy)prop-1-en-2-yl)-4,4,5,5-tetramethyl-1,3,2-dioxaborolane (15 g, 6.69 mmol) in DCM (1.5 L) was heated at 50° C. for 1 h under nitrogen atmosphere. After 1 h, the reaction mass was cooled to room temperature and 2nd Generation Grubss catalyst (2.1 g, 3.35 mmol), was added to and the reaction mass was stirred at same temperature for 16 h. After 16 h, the reaction mass was evaporated and purified by column purification and product was eluted 10% ethyl acetate in hexanes to afford the product (3.4 g, 25.9% yield) as a yellow liquid. ¹H NMR (400 MHz, CDCl₃) δ 6.58 (s, 1H), 4.78-4.77 (m, 2H), 4.72-4.70 (m, 2H), 1.30 (s, 15H).

Step 4: methyl 2-(2,5-dihydrofuran-3-yl)-5-nitrobenzoate

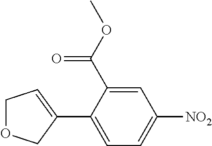 [SMILES: COC(=O)c1cc([N+](=O)[O-])ccc1C1=CCOC1]

To a degassed solution of methyl 2-iodo-5-nitrobenzoate (25 g, 96.1 mmol), 2-(2,5-dihydrofuran-3-yl)-4,4,5,5-tetramethyl-1,3,2-dioxaborolane (19 g, 96.1 mmol, impure) and Na₂CO₃ (3.6 g, 288.4 mmol) in dioxane: water (2: 1, 370 mL) was added Pd(dppf)Cl₂.DCM (7.85 g, 9.61 mmol) and the reaction mass was stirred at 90° C. for 3 h. The mixture was then diluted with water (400 mL) and extracted with EtOAc (3×300 mL). The crude was purified by column chromatography and the product was eluted in 20% EtOAc in hexanes to afford the product (8 g, 39.4% yield) as light yellow semi-solid. ¹H NMR (400 MHz, Chloroform-d) δ 8.68 (d, J=2 Hz, 1H), 8.47 (s, 1H), 8.33 (dd, J=8.4, 2.4 Hz, 1H), 7.50 (d, J=8.4 Hz, 1H), 6.02 (s, 1H), 4.90 (s, 4H), 3.96 (s, 3H).

Step 5: Methyl 5-amino-2-(tetrahydrofuran-3-yl)benzoate

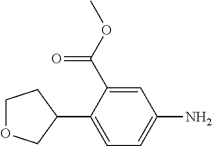 [SMILES: COC(=O)c1cc(N)ccc1C1CCOC1]

In a 3 neck RBF, 10% Pd/C (50% moist, 8 g) and methyl 2-(2,5-dihydrofuran-3-yl)-5-nitrobenzoate (8 g, 32.08 mmol) were added in EtOAc (400 mL) and MeOH (8 mL). The reaction mass was stirred at 80° C. with 20 Kg/cm₂ pressure of hydrogen for 16 h. The mixture was then filtered through celite bed and the filtrate was concentrated under vacuum to afford the product (6.5 g, 31.5% yield) as an yellow semi solid. LCMS: [M+H]⁺=222.1.

Step 6: Methyl 5-bromo-2-(tetrahydrofuran-3-yl)benzoate

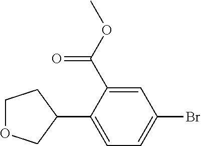 [SMILES: COC(=O)c1cc(Br)ccc1C1CCOC1]

To a solution of methyl 5-amino-2-(tetrahydrofuran-3-yl)benzoate (6 g, 27.1 mmol) in bromoform (18 mL) was dropwise added tert-butylnitrite (12 mL) at room temperature and the reaction mass was stirred for 30 min. The crude product was purified by column chromatography and the product was eluted in 10% EtOAc in hexanes to afford the product (3 g, 38.8% yield) as a yellow semi-solid. LCMS: [M+2]⁺=287.1.

Step 7: 5-bromo-2-(tetrahydrofuran-3-yl)benzoate

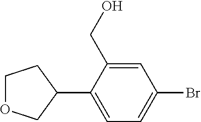 [SMILES: OCc1cc(Br)ccc1C1CCOC1]

To a cooled solution of methyl 5-bromo-2-(tetrahydrofuran-3-yl)benzoate (3.5 g, 12.28 mmol) in dry THF (70 mL) was added LAH (1 M in THF, 18.42 mL, 18.42 mmol) dropwise at 0° C. and the reaction mass was stirred at RT for 3 h. The mixture was quenched with the dil. HCl (50 mL) and extracted with EtOAc (3×100 mL). The combined organics were dried over Na₂SO₄, and concentrated under vacuum to afford the product (2.5 g, 79.21%) as pale yellow liquid. LCMS: [M-18]+2=241.1.

Step 8: 5-bromo-2-(tetrahydrofuran-3-yl)benzaldehyde

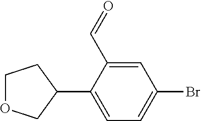 [SMILES: O=Cc1cc(Br)ccc1C1CCOC1]

To a solution of methyl 5-bromo-2-(tetrahydrofuran-3-yl)benzoate (2.5 g, 9.72 mmol) in DCM (50 mL) was added MnO2 (8.5 g, 97.2 mmol) at room temperature and the reaction mass was stirred at room temperature for 48 h. After completion of reaction, reaction mass was filtered through celite bed and the filtrate was concentrate under vacuum to afford crude product. The crude was purified by column chromatography and the product was eluted in 5-10% EtOAc in hexanes to afford the product(1 g, 41% yield) as light yellow semi-solid. LCMS: [M+2]=257.2.

Step 9: 1-(5-bromo-2-(tetrahydrofuran-3-yl)phenyl)-N,N-dimethylmethanamine

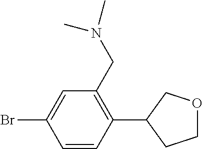 [SMILES: CN(C)Cc1cc(Br)ccc1C1CCOC1]

Prepared from 5-bromo-2-(tetrahydrofuran-3-yl)benzaldehyde (155 mg, 0.608 mmol) and dimethylamine, 2.0M solution in THF (1.215 ml, 2.430 mmol) to give the product as a pale-yellow oil (147 mg, 81% yield). LCMS: [M+H]⁺=284.30 Step 10: 6-(2-amino-5-(3-((dimethylamino)methyl)-4-(tetrahydrofuran-3-yl)phenyl)-6-fluoropyridin-3-yl)-3,4-dihydroisoquinolin-1(2H)-one)

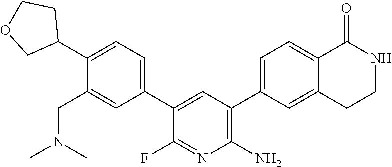 [SMILES: CN(C)Cc1cc(-c2cc(-c3ccc4c(c3)CCNC4=O)c(N)nc2F)ccc1C1CCOC1]

Prepared from 1-(5-bromo-2-(tetrahydrofuran-3-yl)phenyl)-N,N-dimethylmethanamine (54 mg, 0.181 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (90 mg, 0.235 mmol) to yield the title compound as an off white solid (44.5 mg, 51% yield). ¹H NMR (500 MHz, DMSO-d₆) δ 7.9-8.0 (m, 1H), 7.9-7.9 (m, 1H), 7.6-7.6 (m, 1H), 7.4-7.5 (m, 3H), 7.3-7.4 (m, 2H), 6.1-6.4 (m, 2H), 3.9-4.0 (m, 2H), 3.8-3.9 (m, 2H), 3.5-3.6 (m, 1H), 3.5-3.5 (m, 1H), 3.4-3.4 (m, 3H), 2.9-3.0 (m, 2H), 2.3-2.3 (m, 1H), 2.15 (s, 6H), 1.9-2.0 (m, 1H); LCMS: [M+H]+=461.44.

### Example 568: 6-(2-amino-5-(3-((dimethylamino)methyl)-4-(tetrahydrofuran-3-yl)phenyl)-6-fluoropyridin-3-yl)-7-fluoro-3,4-dihydroisoquinolin-1(2H)-one (I-621)

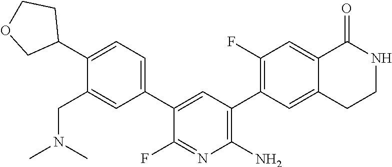 [SMILES: CN(C)Cc1cc(-c2cc(-c3cc4c(cc3F)C(=O)NCC4)c(N)nc2F)ccc1C1CCOC1]

Prepared from 1-(5-bromo-2-(tetrahydrofuran-3-yl)phenyl)-N,N-dimethylmethanamine (50 mg, 0.176 mmol) and 6-(2-amino-6-fluoro-5-(4,4,5,5-tetramethyl-1,3,2-dioxaborolan-2-yl)pyridin-3-yl)-7-fluoro-3,4-dihydroisoquinolin-1(2H)-one (92 mg, 0.229 mmol) to yield the title compound as an off white solid (16 mg, 18% yield). ¹H NMR (500 MHz, DMSO-d₆) δ 8.0-8.2 (m, 1H), 7.6-7.7 (m, 2H), 7.4-7.5 (m, 2H), 7.3-7.4 (m, 2H), 6.2-6.4 (m, 2H), 3.9-4.0 (m, 2H), 3.8-3.9 (m, 2H), 3.5-3.6 (m, 1H), 3.5-3.5 (m, 1H), 3.4-3.4 (m, 3H), 2.93 (br t, 2H, J=6.3 Hz), 2.3-2.3 (m, 1H), 2.1-2.2 (m, 6H), 1.9-2.0 (m, 1H); LCMS: [M+H]⁺=479.43.

### Example 569: 6-(2-amino-5-(5-((dimethylamino)methyl)-2-fluoro-4-morpholinophenyl)-6-fluoropyridin-3-yl)-3,4-dihydroisoquinolin-1(2H)-one (I-622)

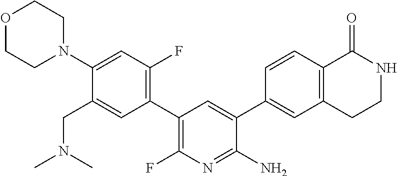 [SMILES: CN(C)Cc1cc(-c2cc(-c3ccc4c(c3)CCNC4=O)c(N)nc2F)c(F)cc1N1CCOCC1]

Prepared from 6-(2-amino-5-bromo-6-fluoropyridin-3-yl)-3,4-dihydroisoquinolin-1(2H)-one (40 mg, 0.119 mmol) and (5-((dimethylamino)methyl)-2-fluoro-4-morpholinophenyl)boronic acid (40.3 mg, 0.143 mmol, prepared starting from 5-bromo-2,4-difluorobenzaldehyde and morpholine using a sequence analogous to that described earlier) to give the title compound (6.7 mg, 11% yield) as an off-white solid. ¹H NMR (500 MHz, DMSO-d₆) δ 7.94 (br. s., 1H), 7.90 (d, J=7.83 Hz, 1H), 7.49 (d, J=9.54 Hz, 1H), 7.36-7.46 (m, 3H), 6.97 (d, J=12.10 Hz, 1H), 6.34 (br. s., 2H), 3.74 (br. s., 4H), 3.39 (br. s., 4H), 2.91-3.01 (m, 6H), 2.16 (s, 6H); LCMS: [M+H]⁺=494.41.

### Example 570: 6-(2-amino-5-(5-((dimethylamino)methyl)-2,3-difluoro-4-morpholinophenyl)-6-fluoropyridin-3-yl)-3,4-dihydroisoquinolin-1(2H)-one (I-623)

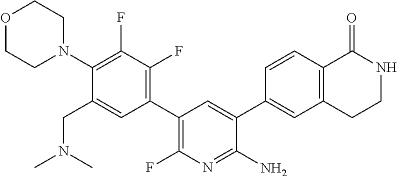 [SMILES: CN(C)Cc1cc(-c2cc(-c3ccc4c(c3)CCNC4=O)c(N)nc2F)c(F)c(F)c1N1CCOCC1]

Prepared from 6-(2-amino-5-bromo-6-fluoropyridin-3-yl)-3,4-dihydroisoquinolin-1(2H)-one (35 mg, 0.104 mmol) and (5-((dimethylamino)methyl)-2,3-difluoro-4-morpholinophenyl)boronic acid (37.5 mg, 0.125 mmol, prepared starting from 5-bromo-2,3,4-trifluorobenzaldehyde and morpholine using methods described previously) to give the title compound (10.7 mg, 20% yield) as an off-white solid. ¹H NMR (500 MHz, DMSO-d₆) δ 7.95 (br. s., 1H), 7.90 (d, J=7.95 Hz, 1H), 7.54 (d, J=9.54 Hz, 1H), 7.41-7.47 (m, 2H), 7.23 (d, J=7.70 Hz, 1H), 6.47 (br. s., 2H), 3.71 (br. s., 4H), 3.46 (s, 2H), 3.39 (td, J=3.18, 6.36 Hz, 2H), 3.09 (br. s., 4H), 2.94 (t, J=6.48 Hz, 2H), 2.16 (s, 6H); LCMS: [M+H]⁺=512.33.

### Example 571: 6-(2-amino-5-(3-(1-(dimethylamino)ethyl)-4-hydroxyphenyl)-6-fluoropyridin-3-yl)-3,4-dihydroisoquinolin-1(2H)-one (I-624)

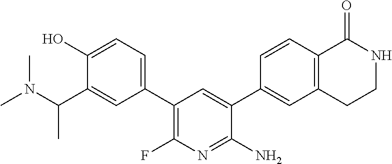 [SMILES: CC(c1cc(-c2cc(-c3ccc4c(c3)CCNC4=O)c(N)nc2F)ccc1O)N(C)C]

Step 1: 4-bromo-2-(1-(dimethylamino)ethyl)phenol

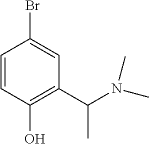

Prepared using procedures similar to earlier examples from 5′-bromo-2′-hydroxyacetophenone 98% (1000 mg, 4.65 mmol) and dimethylamine, 2.0M solution in THF (9.30 ml, 18.60 mmol) to give the product (684 mg, 88% purity). LCMS: [M+H]⁺=244.29.

Step 2: 6-(2-amino-5-(3-(1-(dimethylamino)ethyl)-4-hydroxyphenyl)-6-fluoropyridin-3-yl)-3,4-dihydroisoquinolin-1(2H)-one

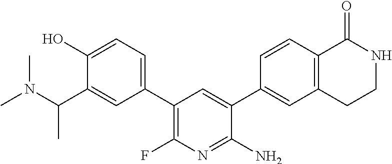 [SMILES: CC(c1cc(-c2cc(-c3ccc4c(c3)CCNC4=O)c(N)nc2F)ccc1O)N(C)C]

Prepared from 4-bromo-2-(1-(dimethylamino)ethyl)phenol (50 mg, 0.205 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (102 mg, 0.266 mmol) to afford the title compound as an off white solid (62 mg, 68% yield). 1H NMR (500 MHz, DMSO-d₆) δ 12.60 (s, 1H), 7.9-8.0 (m, 1H), 7.91 (d, 1H, J=7.9 Hz), 7.6-7.6 (m, 1H), 7.5-7.5 (m, 1H), 7.4-7.5 (m, 1H), 7.3-7.3 (m, 1H), 7.2-7.2 (m, 1H), 6.7-6.8 (m, 1H), 6.1-6.2 (m, 2H), 3.6-3.7 (m, 1H), 3.4-3.4 (m, 2H), 2.9-3.0 (m, 2H), 2.2-2.3 (m, 6H), 1.3-1.3 (m, 3H); LCMS: [M+H]⁺=421.40.

### Example 572: 6-(2-amino-5-(3-(2-aminopropan-2-yl)-4-fluorophenyl)-6-fluoropyridin-3-yl)-3,4-dihydroisoquinolin-1(2H)-one (I-625)

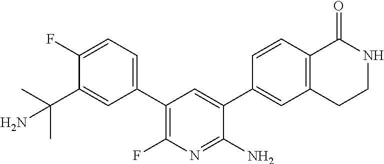 [SMILES: CC(C)(N)c1cc(-c2cc(-c3ccc4c(c3)CCNC4=O)c(N)nc2F)ccc1F]

Step 1: 2-(5-bromo-2-fluorophenyl)propan-2-o

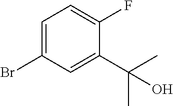

To a 0° C. solution of methyl 5-bromo-2-fluorobenzoate (6.0 g, 25.7 mmol) in anhydrous tetrahydrofuran (50 ml) was added MeMgCl, 3M in THF (21.46 ml, 64.4 mmol) dropwise. The reaction was warmed to room temperature and stirred overnight. The solution was poured into an aqueous saturated solution of ammonium chloride and the organic material was extracted with EtOAc (2×). The combined organic layers were dried over anhydrous Na₂SO₄, filtered and concentrated. The crude material was carried onto the next step without further characterization.

Step 2: N-(2-(5-bromo-2-fluorophenyl)propan-2-yl)-2-chloroacetamide

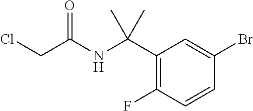 [SMILES: CC(C)(NC(=O)CCl)c1cc(Br)ccc1F]

To a solution of 2-(5-bromo-2-fluorophenyl)propan-2-ol (6.00 g, 25.7 mmol) in chloroacetonitrile 99% (48.9 mL, 772 mmol) was added acetic acid (4.42 mL, 77 mmol). The reaction was cooled to 0° C. and sulfuric acid (4.41 mL, 82 mmol) was added dropwise. The reaction was warmed to room temperature and stirred overnight. The mixture was then poured into ice and extracted with EtOAc. The organic layer was washed with aqueous 1N NaOH(aq) solution and brine, dried over anhydrous Na₂SO₄, and concentrated to give the crude product (7.94 g, 100%) which was carried onto the next step. LCMS: [M+H]⁺=308.03.

Step 3: 2-(5-bromo-2-fluorophenyl)propan-2-amine

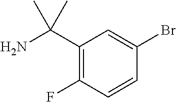

To a solution of N-(2-(5-bromo-2-fluorophenyl)propan-2-yl)-2-chloroacetamide (5.0 g, 16.20 mmol) in ethanol (30 mL) was added concentrated HCl (9.91 ml, 405 mmol) and refluxed overnight. The reaction was concentrated in vacuo and partitioned between DCM and 1 N NaOH(aq). The organic layer was removed, and the aqueous layer was further washed with DCM (2x). The organic layers were combined, dried over Na₂SO₄, concentrated, and dried under vacuum to obtain the product (1.54 g, 41% yield) as a yellow oil. LCMS: [M+H]⁺=232.11.

Step 4: (3-(2-aminopropan-2-yl)-4-fluorophenyl)boronic acid

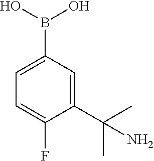

Prepared from 2-(5-bromo-2-fluorophenyl)propan-2-amine (200 mg, 0.862 mmol) to give the boronic acid which was used in the next step without further purification. LCMS: [M+H]⁺=198.21.

Step 5: 6-(2-amino-5-(3-(2-aminopropan-2-yl)-4-fluorophenyl)-6-fluoropyridin-3-yl)-3,4-dihydroisoquinolin-1(2H)-one

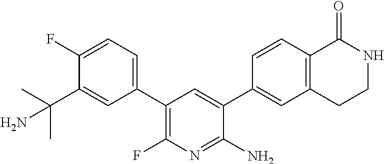 [SMILES: CC(C)(N)c1cc(-c2cc(-c3ccc4c(c3)CCNC4=O)c(N)nc2F)ccc1F]

Prepared from 6-(2-amino-5-bromo-6-fluoropyridin-3-yl)-3,4-dihydroisoquinolin-1(2H)-one (40 mg, 0.119 mmol) and (3-(2-aminopropan-2-yl)-4-fluorophenyl)boronic acid (70.3 mg, 0.357 mmol) to give the title compound (31.5 mg, 64.8% yield) as white solid. ¹H NMR (500 MHz, DMSO-d₆) δ 7.95 (br. s., 1H), 7.91 (d, J=7.95 Hz, 1H), 7.73 (dd, J=1.90, 8.13 Hz, 1H), 7.68 (d, J=10.15 Hz, 1H), 7.46 (d, J=7.95 Hz, 1H), 7.43 (s, 1H), 7.41 (dt, J=2.26, 4.13 Hz, 1H), 7.14 (dd, J=8.44, 12.23 Hz, 1H), 6.29 (s, 2H), 3.40 (dt, J=2.69, 6.42 Hz, 2H), 2.95 (t, J=6.48 Hz, 2H), 2.30 (br. s., 2H), 1.43 (s, 6H); LCMS: [M +H]⁺=409.29.

### Example 573: 6-(2-amino-5-(3-((dimethylamino)methyl)-4-fluorophenyl)-6-fluoropyridin-3-yl)-4-methylisoquinolin-1(2H)-one (I-626)

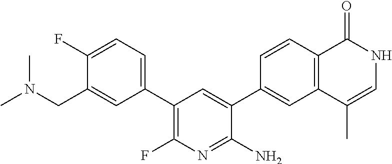 [SMILES: Cc1c[nH]c(=O)c2ccc(-c3cc(-c4ccc(F)c(CN(C)C)c4)c(F)nc3N)cc12]

Prepared from 6-(2-amino-5-bromo-6-fluoropyridin-3-yl)-4-methylisoquinolin-1(2H)-one (50.1 mg, 0.144 mmol) and 1-(2-fluoro-5-(4,4,5,5-tetramethyl-1,3,2-dioxaborolan-2-yl)phenyl)-N,N-dimethylmethanamine (58 mg, 187 mmol, prepared from 1-(5-bromo-2-fluorophenyl)-N,N-dimethylmethanamine) to afford the title compound as an off white solid (34 mg, 53% yield). ¹H NMR (500 MHz, DMSO-d₆)=11.16-11.06 (m, 1H), 8.33-8.24 (m, 1H), 7.76-7.72 (m, 1H), 7.72-7.69 (m, 1H), 7.65-7.61 (m, 1H), 7.57-7.53 (m, 1H), 7.53-7.47 (m, 1H), 7.27-7.19 (m, 1H), 7.09-7.01 (m, 1H), 6.47-6.34 (m, 2H), 3.50-3.44 (m, 2H), 2.28-2.23 (m, 3H), 2.20-2.13 (m, 6H); LCMS: [M+H]⁺=421.28.

### Example 574: 6-(2-amino-5-(3-((dimethylamino)methyl)phenyl)-6-fluoropyridin-3-yl)-4-methylisoquinolin-1(2H)-one (I-627)

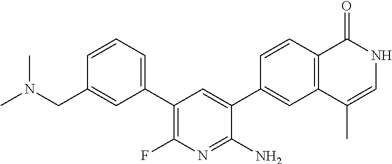 [SMILES: Cc1c[nH]c(=O)c2ccc(-c3cc(-c4cccc(CN(C)C)c4)c(F)nc3N)cc12]

Prepared from N,N-dimethyl-1-(3-(4,4,5,5-tetramethyl-1,3,2-dioxaborolan-2-yl)phenyl)methanamine (55 mg, 0.190 mmol) and 6-(2-amino-5-bromo-6-fluoropyridin-3-yl)-4-methylisoquinolin-1(2H)-one (50.8 mg, 0.146 mmol) to afford the title compound as a beige solid (31.5 mg, 51% yield). ¹H NMR (500 MHz, DMSO-d₆) δ=11.09 (br d, J=5.3 Hz, 1H), 8.34-8.24 (m, 1H), 7.78-7.69 (m, 2H), 7.66-7.58 (m, 1H), 7.48-7.42 (m, 2H), 7.40-7.32 (m, 1H), 7.25-7.19 (m, 1H), 7.07-7.01 (m, 1H), 6.44-6.30 (m, 2H), 3.47-3.39 (m, 2H), 2.29-2.22 (m, 3H), 2.19-2.13 (m, 6H); LCMS: [M+H]⁺=403.41.

### Example 575: (R)-6-(2-amino-5-(4-(dimethylamino)chroman-6-yl)-6-fluoropyridin-3-yl)-4-methylisoquinolin-1(2H)-one (I-628)

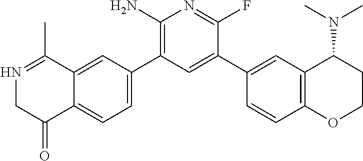 [SMILES: CC1=NCC(=O)c2ccc(-c3cc(-c4ccc5c(c4)[C@H](N(C)C)CCO5)c(F)nc3N)cc21]

Step 1 (S,E)-N-(6-bromochroman-4-ylidene)-2-methylpropane-2-sulfinamide

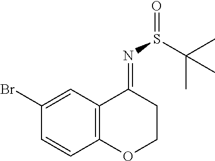 [SMILES: CC(C)(C)[S@](=O)/N=C1\CCOc2ccc(Br)cc21]

6-Bromo-2,3-dihydro-4h-chromen-4-one (5 g, 22.02 mmol) was mixed with (S)-(−)-2-Methyl-2-propanesulfinamide (6.67 g, 55.1 mmol) in Toluene (50 ml), then titanium(IV) ethoxide (5.54 ml, 26.4 mmol) was added. The reaction mixture was stirred at 90° C. for 2 hrs and quenched with ice-water and EtOAc. a lot of solid came out and filtered. The solid was rinsed with EtOAc several time. The organic layer of the filtrate was dried with MgSO4, concentrated with silica gel purified by column chromatography, eluted with 0-30% EtOAc in hexanes to give the product (6.697 g, 92% yield) as a yellow solid. LCMS: [M+H]⁺=330.19.

Step 2: (S)—N—((S)-6-bromochroman-4-yl)-2-methylpropane-2-sulfinamide and (S)—N—((R)-6-bromochroman-4-yl)-2-methylpropane-2-sulfinamide

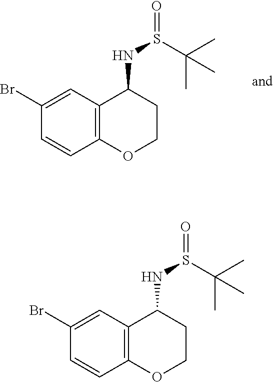 [SMILES: CC(C)(C)[S@](=O)N[C@H]1CCOc2ccc(Br)cc21 ; CC(C)(C)[S@](=O)N[C@@H]1CCOc2ccc(Br)cc21]

(S,E)-N-(6-bromochroman-4-ylidene)-2-methylpropane-2-sulfinamide (3.3 g, 9.99 mmol) was mixed with THF (40 mL) and water (0.6 ml), then was cooled with dry ice-acetone bath (<−50° C.). Sodium borohydride (1.134 g, 30.0 mmol) was added and kept stirring 30 min, then slowly warmed to r.t. for 2 hrs. The reaction mixture quenched with sat-NH₄Cl and extracted with EtOAc. The organic layer was washed with brine, concentrated with silica gel. Purification column chromatography. eluted with 20-25% EtOAc in hexanes to give the product (2.557 g, 77% yield) 890 mg as a white solid. LCMS: [M+H]⁺=332.06.

Step 3: (R)-6-bromochroman-4-amine hydrochloride

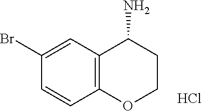 [SMILES: N[C@@H]1CCOc2ccc(Br)cc21 ; Cl]

(R)-6-bromochroman-4-amine hydrochloride (395 mg, 117% yield) was prepared from (S)—N—((R)-6-bromochroman-4-yl)-2-methylpropane-2-sulfinamide (423 mg, 1.273 mmol) reacted with 4.0 M hydrochloric acid in dioxane (3.18 mL, 12.73 mmol) in methanol (5 mL) at r.t. for 90 min. LCMS: [M−HCl—NH₃+H]⁺=211.18.

Step 4: (R)-6-bromo-N,N-dimethylchroman-4-amine

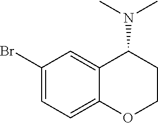

Prepared from (R)-6-bromochroman-4-amine hydrochloride (395 mg, 1.493 mmol) reacted with 37% formaldehyde solution in water (485 mg, 5.97 mmol), sodium acetate (122 mg, 1.493 mmol) and sodium triacetoxyborohydride (949 mg, 4.48 mmol) in methanol (2 ml) and dichloromethane (DCM) (8 ml) at r.t. For 1 h to give the product (374 mg, 98% yield). [M+H]⁺=256.20.

Step 5: (R)—N,N-dimethyl-6-(4,4,5,5-tetramethyl-1,3,2-dioxaborolan-2-yl)chroman-4-amine hydrochloride

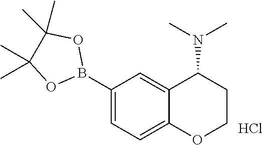

Prepared from (R)-6-bromo-N,N-dimethylchroman-4-amine to give the product (87% yield). LCMS=[M−HCl+H]⁺=304.36.

Step 6: (R)-6-(2-amino-5-(4-(dimethylamino)chroman-6-yl)-6-fluoropyridin-3-yl)-4-methylisoquinolin-1(2H)-one

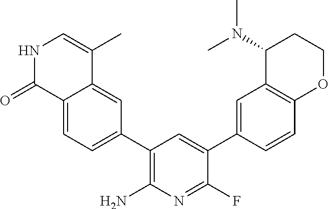 [SMILES: Cc1c[nH]c(=O)c2ccc(-c3cc(-c4ccc5c(c4)[C@H](N(C)C)CCO5)c(F)nc3N)cc12]

Prepared from 6-(2-amino-5-bromo-6-fluoropyridin-3-yl)-4-methylisoquinolin-1(2H)-one (35.9 mg, 0.103 mmol) to give the title compound (23.5 mg, 51.0% yield). ¹H NMR (500 MHz, DMSO-d₆) δ=11.09 (br d, J=5.3 Hz, 1H), 8.28 (d, J=8.2 Hz, 1H), 7.72 (s, 1H), 7.66-7.59 (m, 2H), 7.55 (s, 1H), 7.31 (br d, J=8.6 Hz, 1H), 7.04 (s, 1H), 6.79 (d, J=8.4 Hz, 1H), 6.27 (s, 2H), 4.39-4.28 (m, 1H), 4.16-4.06 (m, 1H), 3.80 (dd, J=5.6, 8.3 Hz, 1H), 2.24 (s, 3H), 2.21 (s, 6H), 2.02-1.87 (m, 2H); [M+H]⁺=445.35.

### Example 576: (S)-6-(2-amino-5-(3-(dimethylamino)-2,3-dihydrobenzofuran-5-yl)-6-fluoropyridin-3-yl)-4-methylisoquinolin-1(2H)-one (I-629)

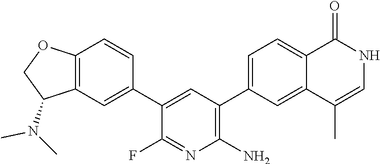 [SMILES: Cc1c[nH]c(=O)c2ccc(-c3cc(-c4ccc5c(c4)[C@H](N(C)C)CO5)c(F)nc3N)cc12]

Step 1: (S)-6-(2-amino-5-(3-(dimethylamino)-2,3-dihydrobenzofuran-5-yl)-6-fluoropyridin-3-yl)-4-methylisoquinolin-1(2H)-one

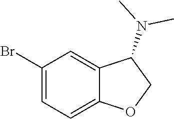

In a similar manner, (S)-5-bromo-N,N-dimethyl-2,3-dihydrobenzofuran-3-amine (0.99 g, 93% yield) was prepared from (S)-5-Bromo-2,3-dihydro-benzofuran-3-ylamine hydrochloride (1.1012 g, 4.40 mmol) and 37% formaldehyde solution in water (1.427 g, 17.58 mmol), sodium acetate (0.361 g, 4.40 mmol) and sodium triacetoxyborohydride (2.79 g, 13.19 mmol) in dichloromethane (20 mL) and methanol (5 mL) at r.t. for 30 min to give the product (0.99 g, 93% yield). LCMS: [M+H]⁺=242.23

Step 2: (S)—N,N-dimethyl-5-(4,4,5,5-tetramethyl-1,3,2-dioxaborolan-2-yl)-2,3-dihydrobenzofuran-3-amine

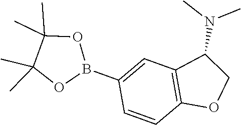

Prepared from (S)-5-bromo-N,N-dimethyl-2,3-dihydrobenzofuran-3-amine (0.99 g, 4.09 mmol) and bis(pinacolato)diboron (1.246 g, 4.91 mmol), potassium acetate (1.204 g, 12.27 mmol), and [1,1′-bis(diphenylphosphino)ferrocene] dichloropalladium(II) (0.299 g, 0.409 mmol) in 1,4-dioxane (20 mL) at 90-95° C. overnight to give the product (0.94 g, 79% yield). LCMS: [M+H]⁺=290.31

Step 3: (S)-6-(2-amino-5-(3-(dimethylamino)-2,3-dihydrobenzofuran-5-yl)-6-fluoropyridin-3-yl)-4-methylisoquinolin-1(2H)-one

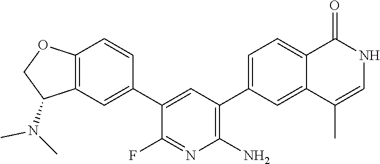 [SMILES: Cc1c[nH]c(=O)c2ccc(-c3cc(-c4ccc5c(c4)[C@H](N(C)C)CO5)c(F)nc3N)cc12]

Prepared from 6-(2-amino-5-bromo-6-fluoropyridin-3-yl)-4-methylisoquinolin-1(2H)-one (42.1 mg, 0.121 mmol) and (S)—N,N-dimethyl-5-(4,4,5,5-tetramethyl-1,3,2-dioxaborolan-2-yl)-2,3-dihydrobenzofuran-3-amine (35 mg, 0.121 mmol) to give the title compound (21.7 mg, 42% yield). ¹H NMR (500 MHz, DMSO-d₆) δ=11.09 (br d, J=5.5 Hz, 1H), 8.28 (d, J=8.3 Hz, 1H), 7.74 (s, 1H), 7.67 (d, J=10.3 Hz, 1H), 7.65-7.61 (m, 1H), 7.48 (s, 1H), 7.40 (d, J=8.4 Hz, 1H), 7.08-7.02 (m, 1H), 6.86 (d, J=8.3 Hz, 1H), 6.28 (s, 2H), 4.54 (dd, J=3.1, 9.9 Hz, 1H), 4.50-4.44 (m, 1H), 4.42-4.34 (m, 1H), 2.24 (s, 3H), 2.12 (s, 6H). LCMS: [M+H]⁺=431.43.

### Example 577: (S)-6-(2-amino-5-(3-(dimethylamino)-2,3-dihydrobenzofuran-5-yl)-6-fluoropyridin-3-yl)-4-fluoroisoquinolin-1(2H)-one (I-630)

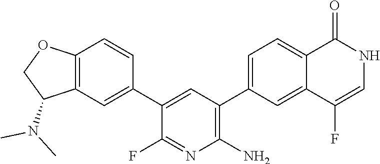 [SMILES: CN(C)[C@@H]1COc2ccc(-c3cc(-c4ccc5c(=O)[nH]cc(F)c5c4)c(N)nc3F)cc21]

Prepared from 6-(2-amino-5-bromo-6-fluoropyridin-3-yl)-4-fluoroisoquinolin-1(2H)-one (37.8 mg, 0.107 mmol) and (S)—N,N-dimethyl-5-(4,4,5,5-tetramethyl-1,3,2-dioxaborolan-2-yl)-2,3-dihydrobenzofuran-3-amine hydrochloride (35 mg, 0.107 mmol) to give the title compound (21.5 mg, 46.0% yield). ¹H NMR (500 MHz, DMSO-d₆) δ=11.17 (br s, 1H), 8.27 (dd, J=1.7, 8.3 Hz, 1H), 7.84 (d, J=1.1 Hz, 1H), 7.75 (dd, J=1.5, 8.3 Hz, 1H), 7.69 (d, J=10.1 Hz, 1H), 7.50 (s, 1H), 7.46-7.37 (m, 2H), 6.87 (d, J=8.4 Hz, 1H), 6.33 (s, 2H), 4.55 (dd, J=3.1, 9.9 Hz, 1H), 4.50-4.45 (m, 1H), 4.43-4.34 (m, 1H), 2.12 (s, 6H); LCMS: [M+H]⁺=435.38.

### Example 578: (R)-6-(2-amino-5-(4-(dimethylamino)chroman-6-yl)-6-fluoropyridin-3-yl)-4-fluoroisoquinolin-1(2H)-one (I-631)

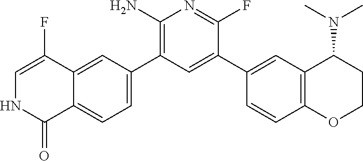 [SMILES: CN(C)[C@@H]1CCOc2ccc(-c3cc(-c4ccc5c(=O)[nH]cc(F)c5c4)c(N)nc3F)cc21]

Prepared from 6-(2-amino-5-bromo-6-fluoropyridin-3-yl)-4-fluoroisoquinolin-1(2H)-one (36.3 mg, 0.103 mmol) and (R)—N,N-dimethyl-6-(4,4,5,5-tetramethyl-1,3,2-dioxaborolan-2-yl)chroman-4-amine hydrochloride (35 mg, 0.103 mmol) to give the title compound (15.3 mg, 32% yield). ¹H NMR (500 MHz, DMSO-d₆) δ=11.15 (br s, 1H), 8.26 (dd, J=1.7, 8.3 Hz, 1H), 7.81 (s, 1H), 7.72 (dd, J=1.2, 8.3 Hz, 1H), 7.63 (d, J=10.1 Hz, 1H), 7.55 (s, 1H), 7.42 (br d, J=5.4 Hz, 1H), 7.31 (br d, J=8.3 Hz, 1H), 6.78 (d, J=8.4 Hz, 1H), 6.32 (s, 2H), 4.37-4.28 (m, 1H), 4.17-4.06 (m, 1H), 3.80 (dd, J=5.6, 8.3 Hz, 1H), 2.20 (s, 6H), 2.03-1.86 (m, 2H); LCMS: [M+H]⁺=449.30.

### Example 579: 5-(6-amino-2-fluoro-5-(1-oxo-1,2,3,4-tetrahydroisoquinolin-6-yl)pyridin-3-yl)-2-methoxybenzonitrile (I-632)

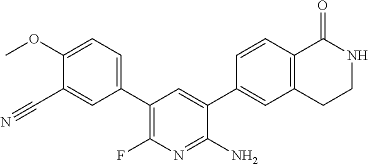 [SMILES: COc1ccc(-c2cc(-c3ccc4c(c3)CCNC4=O)c(N)nc2F)cc1C#N]

Prepared from 6-(2-amino-5-bromo-6-fluoropyridin-3-yl)-3,4-dihydroisoquinolin-1(2H)-one (40 mg, 0.119 mmol) and 2-methoxy-5-(4,4,5,5-tetramethyl-1,3,2-dioxaborolan-2-yl)benzonitrile (37 mg, 0.143 mmol, prepared from 5-bromo-2-methoxybenzonitrile using similar procedures to those described earlier) to give the title compound (39 mg, 84% yield) as a white solid. ¹H NMR (500 MHz, DMSO-d₆) δ 7.94 (d, J=2.20 Hz, 2H), 7.91 (d, J=7.95 Hz, 1H), 7.86 (d, J=8.80 Hz, 1H), 7.71 (d, J=10.27 Hz, 1H), 7.49 (dd, J=1.47, 7.95 Hz, 1H), 7.46 (s, 1H), 7.30 (d, J=9.05 Hz, 1H), 6.38 (s, 2H), 3.94 (s, 3H), 3.40 (dt, J=2.63, 6.51 Hz, 2H), 2.95 (t, J=6.54 Hz, 2H); LCMS: [M+H]⁺=389.31.

### Example 580: 6-(2-amino-5-(3-((ethyl(methyl)amino)methyl)-4-methoxyphenyl)-6-fluoropyridin-3-yl)-4-fluoroisoquinolin-1(2H)-one (I-633)

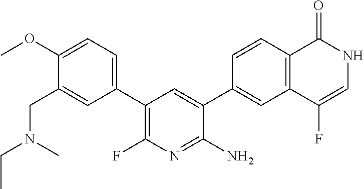 [SMILES: CCN(C)Cc1cc(-c2cc(-c3ccc4c(=O)[nH]cc(F)c4c3)c(N)nc2F)ccc1OC]

Step 1: N-(5-bromo-2-methoxybenzyl)-N-methylethanamine

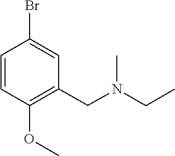

Prepared using 5-bromo-2-methoxybenzaldehyde (335 mg, 1.558 mmol) and N-ethylmethylamine (0.535 ml, 6.23 mmol) to give the product as a colorless oil (347 mg, 82% yield). LCMS: [M+H]⁺=258.27.

Step 2: N-(2-methoxy-5-(4,4,5,5-tetramethyl-1,3,2-dioxaborolan-2-yl)benzyl)-N-methylethanamine

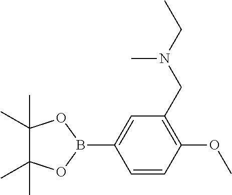

Prepared from N-(5-bromo-2-methoxybenzyl)-N-methylethanamine (240 mg, 0.837 mmol) to give the product as a black solid which was taken to the next step without any purification. (255 mg, 90% yield); LCMS: [M+H]⁺=306.36.

Step 3: 6-(2-amino-5-(3-((ethyl(methyl)amino)methyl)-4-methoxyphenyl)-6-fluoropyridin-3-yl)-4-fluoroisoquinolin-1(2H)-one

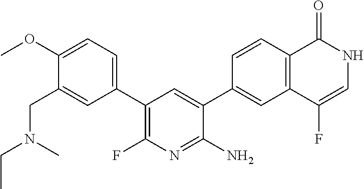 [SMILES: CCN(C)Cc1cc(-c2cc(-c3ccc4c(=O)[nH]cc(F)c4c3)c(N)nc2F)ccc1OC]

Prepared from 6-(2-amino-5-bromo-6-fluoropyridin-3-yl)-4-fluoroisoquinolin-1(2H)-one (51.9 mg, 0.147 mmol) and N-(2-methoxy-5-(4,4,5,5-tetramethyl-1,3,2-dioxaborolan-2-yl)benzyl)-N-methylethanamine (60 mg, 0.177 mmol) to afford the title compound as a beige solid (28 mg, 40% yield). ¹H NMR (500 MHz, DMSO-d₆) δ ppm 11.17 (br. s., 1H), 8.28 (d, J=8.07 Hz, 1H), 7.83 (s, 1H), 7.74 (d, J=8.31 Hz, 1H), 7.67 (d, J=10.03 Hz, 1H), 7.50 (br. s., 1H), 7.43 (br. s., 2H), 7.03 (d, J=8.44 Hz, 1H), 6.34 (br. s., 2H), 3.81 (s, 3H), 3.48 (br. s., 2H), 2.37-2.45 (m, 2H), 2.07-2.21 (m, 3H), 1.03 (br. s., 3H); LCMS: [M+H]⁺=451.55

### Example 581: 6-(2-amino-5-(3-((ethyl(methyl)amino)methyl)-4-methoxyphenyl)-6-fluoropyridin-3-yl)-4-methylisoquinolin-1(2H)-one (I-634)

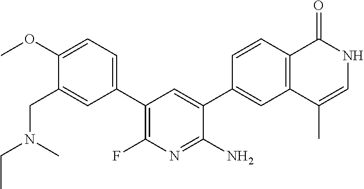 [SMILES: CCN(C)Cc1cc(-c2cc(-c3ccc4c(=O)[nH]cc(C)c4c3)c(N)nc2F)ccc1OC]

Prepared from 6-(2-amino-5-bromo-6-fluoropyridin-3-yl)-4-methylisoquinolin-1(2H)-one (51.3 mg, 0.147 mmol) and N-(2-methoxy-5-(4,4,5,5-tetramethyl-1,3,2-dioxaborolan-2-yl)benzyl)-N-methylethanamine (60 mg, 0.177 mmol) to afford the title compound as a beige solid (28 mg, 40% yield). ¹H NMR (500 MHz, DMSO-d₆) ppm 11.10 (br. s., 1H), 8.29 (d, J=8.19 Hz, 1H), 7.73 (s, 1H), 7.67 (d, J=9.90 Hz, 1H), 7.63 (d, J=8.07 Hz, 1H), 7.36-7.56 (m, 1H), 7.36-7.56 (m, 2H), 7.05 (br. s., 2H), 6.29 (br. s., 2H), 3.81 (br. s., 3H), 3.48 (br. s., 2H), 2.36-2.44 (m, 2H), 2.25 (s, 3H), 2.03-2.23 (m, 3H), 0.94-1.10 (m, 3H); LCMS: [M+H]⁺=447.35

### Example 582: 8-(2-amino-5-(4-(4-(cyclopropylmethyl)piperazin-1-yl)phenyl)-6-fluoropyridin-3-yl)-6-fluoro-3,4-dihydrobenzo[f][1,4]oxazepin-5(2H)-one (I-635)

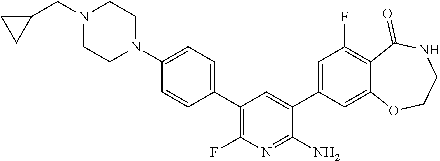 [SMILES: Nc1nc(F)c(-c2ccc(N3CCN(CC4CC4)CC3)cc2)cc1-c1cc(F)c2c(c1)OCCNC2=O]

To a degassed suspension of 8-bromo-6-fluoro-3,4-dihydrobenzo[f][1,4]oxazepin-5(2H)-one (43 mg, 0.165 mmol), 5-(4-(4-(cyclopropylmethyl)piperazin-1-yl)phenyl)-6-fluoro-3-(4,4,5,5-tetramethyl-1,3,2-dioxaborolan-2-yl)pyridin-2-amine (90 mg, 0.198 mmol), Potassium phosphate tribasic (105 mg, 0.496 mmol) and XPhos Pd G2 (15.61 mg, 0.020 mmol) was added a 5:1 mixture of Acetonitrile (4 ml): Water (0.800 ml). The system was flushed with nitrogen then heated at 90° C. overnight on the stir plate. The reaction was concentrated onto celite and purified by silica gel chromatography to give the title compound (3.2 mg, 3.83% yield) as an off-white solid. ¹H NMR (500 MHz, DMSO-d₆) δ ppm 8.47 (t, J=5.87 Hz, 1H), 7.60 (d, J=10.15 Hz, 1 H), 7.40 (d, J=8.07 Hz, 2H), 7.20 (d, J=10.64 Hz, 1H), 7.04 (s, 1H), 6.97 (d, J=8.80 Hz, 2 H), 6.29 (s, 2H), 4.22 (t, J=5.32 Hz, 2H), 3.16-3.20 (m, 4H), 2.56-2.60 (m, 4H), 2.22 (d, J=6.60 Hz, 2H), 0.86 (d, J=3.42 Hz, 1H), 0.45-0.50 (m, 2H), 0.10 (q, J=4.65 Hz, 2H); LCMS: [M+H]⁺=506.66.

### Example 583: 8-(2-amino-6-fluoro-5-(4-(4-isopropylpiperazin-1-yl)phenyl)pyridin-3-yl)-6-fluoro-3,4-dihydrobenzo[f][1,4]oxazepin-5(2H)-one (I-636)

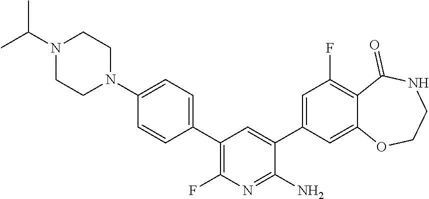 [SMILES: CC(C)N1CCN(c2ccc(-c3cc(-c4cc(F)c5c(c4)OCCNC5=O)c(N)nc3F)cc2)CC1]

Prepared from 8-bromo-6-fluoro-3,4-dihydrobenzo[f][1,4]oxazepin-5(2H)-one (39.5 mg, 0.152 mmol) and 6-fluoro-5-(4-(4-isopropylpiperazin-1-yl)phenyl)-3-(4,4,5,5-tetramethyl-1,3,2-dioxaborolan-2-yl)pyridin-2-amine (80 mg, 0.182 mmol) to give 8-(2-amino-6-fluoro-5-(4-(4-isopropylpiperazin-1-yl)phenyl)pyridin-3-yl)-6-fluoro-3,4-dihydrobenzo[f][1,4]oxazepin-5(2H)-one (15.6 mg, 20.81% yield) as beige solid. ¹H NMR (500 MHz, DMSO-d₆) δ ppm 8.47 (t, J=5.93 Hz, 1H), 7.60 (d, J=10.15 Hz, 1H), 7.40 (d, J=8.19 Hz, 2H), 7.19 (d, J=10.64 Hz, 1H), 7.04 (s, 1H), 6.96 (d, J=8.68 Hz, 2H), 6.29 (s, 2 H), 4.22 (t, J=5.32 Hz, 2H), 3.28-3.31 (m, 2H), 3.15 (br. s., 4H), 2.68 (br. s., 1H), 2.59 (br. s., 4H), 1.01 (d, J=6.24 Hz, 6H); LCMS: [M+H]⁺=494.63.

### Example 584: 5-(2-amino-6-fluoro-5-(4-(4-isopropylpiperazin-1-yl)phenyl)pyridin-3-yl)-7-fluoroisoindolin-1-one (I-637)

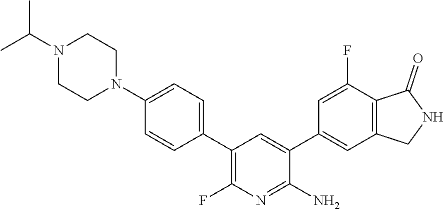 [SMILES: CC(C)N1CCN(c2ccc(-c3cc(-c4cc(F)c5c(c4)CNC5=O)c(N)nc3F)cc2)CC1]

Prepared from 5-bromo-7-fluoroisoindolin-1-one (35 mg, 0.152 mmol) and 6-fluoro-5-(4-(4-isopropylpiperazin-1-yl)phenyl)-3-(4,4,5,5-tetramethyl-1,3,2-dioxaborolan-2-yl)pyridin-2-amine (80 mg, 0.183 mmol) to give 5-(2-amino-6-fluoro-5-(4-(4-isopropylpiperazin-1-yl)phenyl)pyridin-3-yl)-7-fluoroisoindolin-1-one (1.5 mg, 2.1% yield) as an off-white solid. ¹H NMR (500 MHz, DMSO-d₆) δ ppm 8.57 (br. s., 1H), 7.63 (d, J=10.03 Hz, 1H), 7.50 (s, 1H), 7.42 (d, J=7.70 Hz, 2H), 7.34 (d, J=10.39 Hz, 1H), 6.99 (d, J=7.70 Hz, 2H), 6.28 (br. s., 2H), 4.41 (br. s., 2H), 3.11-3.23 (m, 4H), 2.81-2.90 (m, 1H), 2.59-2.79 (m, 4H), 1.08 (br. s., 6H); LCMS: [M+H]⁺=464.55.

### Example 585: 6-(2-amino-6-fluoro-5-(3-(3-((4-methyl-4H-1,2,4-triazol-3-yl)methyl)oxetan-3-yl)phenyl)pyridin-3-yl)-3,4-dihydroisoquinolin-1(2H)-one I-638

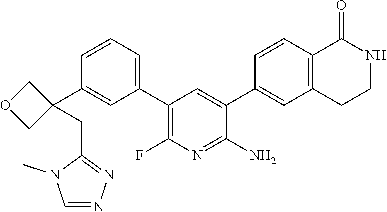 [SMILES: Cn1cnnc1CC1(c2cccc(-c3cc(-c4ccc5c(c4)CCNC5=O)c(N)nc3F)c2)COC1]

Step 1: ethyl 2-(3-(3-nitrophenyl)oxetan-3-yl)acetate

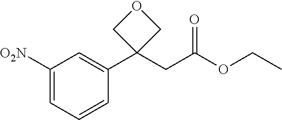 [SMILES: CCOC(=O)CC1(c2cccc([N+](=O)[O-])c2)COC1]

To a stirred solution of [Rh(COD)Cl]₂ (1.4 g, 0.04 mmol) in 1,4-Dioxane (100 mL) was added 1.5M KOH solution (47 mL, 70.4 mmol) stirred for 15 mins and then were added 3-nitrophenylboronic acid (10 g, 70.4 mmol) and ethyl 2-(oxetan-3-ylidene)acetate (23.5 g, 140.9 mmol) at RT for 16 h. The reaction mixture was quenched with ice water (200 mL) and extracted with ethyl acetate (2×200 mL), the combined organic layers were dried over Na₂SO₄ and concentrated under reduced pressure gave the crude product which was purified by sgc (100-200 mesh) eluting with 15-20% ethyl acetate in pet ether to afford the product (6 g, 32%) as a pale brown sticky solid. LCMS: [M+H]⁺=266.12

Step 2: 2-(3-(3-nitrophenyl)oxetan-3-yl)acetohydrazide

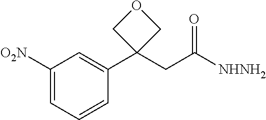 [SMILES: NNC(=O)CC1(c2cccc([N+](=O)[O-])c2)COC1]

To a stirred solution of ethyl 2-(3-(3-nitrophenyl)oxetan-3-yl)acetate (6 g, 22.6 mmol) in ethanol (60 mL) was added hydrazine hydrate (7.1 mL, 226.4 mmol) at RT. The reaction mixture was stirred at 85° C. for 16 h before cooling to RT. The reaction mixture was concentrated under reduced pressure to give crude compound. Which was washed with 10% ethyl acetate in pet ether (50 mL) to afford the product (6 g, 92%) as a brown sticky solid. TLC system: 10% MeOH in DCM; R_{f}=0.7.

Step 3: N-methyl-2-(2-(3-(3-nitrophenyl)oxetan-3-yl)acetyl)hydrazine-1-carbothioamide

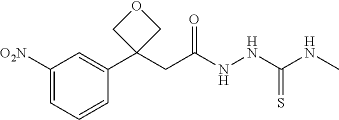 [SMILES: CNC(=S)NNC(=O)CC1(c2cccc([N+](=O)[O-])c2)COC1]

To a stirred solution of 2-(3-(3-nitrophenyl)oxetan-3-yl)acetohydrazide (6 g, 23.9 mmol) in THF (120 mL) was added isothiocyanatomethane (3.5 g. 47.8 mmol) and TEA (3.2 mL, 23.9 mmol) at 0° C. The reaction mixture was stirred at RT for 16 h. The precipitate was filtered and washed with THF (30 mL) and dried under vacuum to afford the product (4 g, 52%) as a pale brown solid. LCMS: [M+H]⁺=325.17.

Step 4: 4-methyl-5-((3-(3-nitrophenyl)oxetan-3-yl)methyl)-4H-1,2,4-triazole-3-thiol

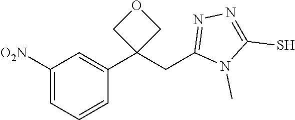 [SMILES: Cn1c(S)nnc1CC1(c2cccc([N+](=O)[O-])c2)COC1]

To a stirred solution of N-methyl-2-(2-(3-(3-nitrophenyl)oxetan-3-yl)acetyl)hydrazine-1-carbothioamide (4 g, 12.3 mmol) in 1M Aq. NaOH solution (185 mL, 185.2 mmol) at RT stirred for 16 h. The reaction mixture was acidified with 1N HCl pH up to 5.0 at 15 to 20° C. and stirred for 30 min. Solid compound was filtered and washed with water (30 mL) and dried under vacuum to afford the product (3 g, 80%) as a pale brown solid. LCMS: [M+H]⁺=307.11

Step 5: 4-methyl-3-((3-(3-nitrophenyl)oxetan-3-yl)methyl)-4H-1,2,4-triazole

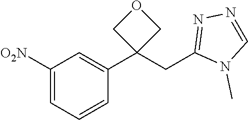 [SMILES: Cn1cnnc1CC1(c2cccc([N+](=O)[O-])c2)COC1]

To a solution of 4-methyl-5-((3-(3-nitrophenyl)oxetan-3-yl)methyl)-4H-1,2,4-triazole-3-thiol (3 g, 9.8 mmol) in water (30 mL) was added NaNO₂ (6.8 g, 98.0 mmol) at RT followed by addition of HNO₃ (4.1 mL, 98.0 mmol) dropwise using an addition funnel for 15 min at 0° C. and stirred for 1 h at same temperature. The reaction mass was basified with saturated NaHCO₃ solution pH up to 8.0. The reaction mass was extracted with EtOAc (2×200 mL), the combined organic layers was dried over Na₂SO₄ and concentrated to afford the product (2.4 g, 89%) as a pale brown solid. LCMS: [M+H]⁺=275.09.

Step 6: 3-(3-((4-methyl-4H-1,2,4-triazol-3-yl)methyl)oxetan-3-yl)aniline

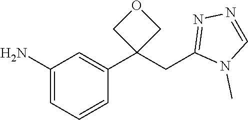 [SMILES: Cn1cnnc1CC1(c2cccc(N)c2)COC1]

To a solution of 4-methyl-3-((3-(3-nitrophenyl)oxetan-3-yl)methyl)-4H-1,2,4-triazole (2.4 g, 8.8 mmol) in ethanol (48 mL) were added Fe (2.45 g, 43.8 mmol) and NH₄Cl (2.34 g, 43.8 mmol) at RT. The reaction mass was stirred at 80° C. for 2 h before cooling to RT. The reaction mixture was filtered through celite pad and washed with ethanol (2×100 mL). The filtrate was concentrated under reduced pressure to give crude compound. Which was purified by column chromatography (silica gel, 100-200 mesh), using 6 to 10% MeOH in DCM as an eluent to afford the product (1.8 g, 84% yield) as a light brown solid. LCMS: [M +H]⁺=245.34.

Step 7: 3-((3-(3-bromophenyl)oxetan-3-yl)methyl)-4-methyl-4H-1,2,4-triazole

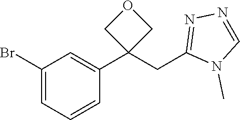 [SMILES: Cn1cnnc1CC1(c2cccc(Br)c2)COC1]

To a solution of 3-(3-((4-methyl-4H-1,2,4-triazol-3-yl)methyl)oxetan-3-yl)aniline (4.2 g, 15.3 mmol) in ACN (42 mL) were added CuBr₂ (3.42 g, 15.3 mmol) and t-BuNO₂ (4.55 mL, 38.3 mmol) at 0° C. The reaction mass was stirred at 70° C. for 5 h before cooling to RT. The reaction mixture was quenched with ice cold water (200 mL). Then, the reaction mixture was filtered through celite pad and washed with ethyl acetate (2×100 mL). The filtrate was extracted with ethyl acetate (2×100 mL). The combined organic layer was dried with sodium sulphate and concentrated under reduced pressure to give crude compound. Which was purified by column chromatography (silica gel, 100-200 mesh), by using 5 to 7% MeOH in DCM as an eluent to afford the crude product (1.6 g, 70% pure by LCMS) as a light green sticky solid. It was again purified by reverse phase column by using 0 to 30% 0.1% TFA in water: ACN as an eluent to afford the purified product (1.3 g, 28%) as an off white solid.; LCMS: [M+H]⁺=308.12.

Step 8: 6-(2-amino-6-fluoro-5-(3-(3-((4-methyl-4H-1,2,4-triazol-3-yl)methyl)oxetan-3-yl)phenyl)pyridin-3-yl)-3,4-dihydroisoquinolin-1(2H)-one I-638

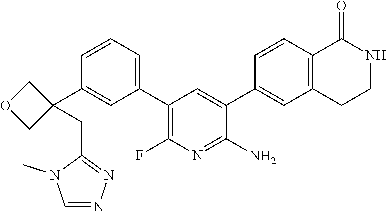 [SMILES: Cn1cnnc1CC1(c2cccc(-c3cc(-c4ccc5c(c4)CCNC5=O)c(N)nc3F)c2)COC1]

Prepared from 6-(2-amino-6-fluoro-5-(4,4,5,5-tetramethyl-1,3,2-dioxaborolan-2-yl)pyridin-3-yl)-3,4-dihydroisoquinolin-1(2H)-one (22.38 mg, 0.058 mmol) and 3-((3-(3-bromophenyl)oxetan-3-yl)methyl)-4-methyl-4H-1,2,4-triazole (15 mg, 0.049 mmol) to give 6-(2-amino-6-fluoro-5-(3-(3-((4-methyl-4H-1,2,4-triazol-3-yl)methyl)oxetan-3-yl)phenyl)pyridin-3-yl)-3,4-dihydroisoquinolin-1(2H)-one (15.3 mg, 0.032 mmol, 64.9% yield) as a white solid. ¹H NMR (500 MHz, DMSO-d₆) δ ppm 8.19 (s, 1H), 7.94 (br. s., 1H), 7.92 (d, J=7.95 Hz, 1H), 7.52 (d, J=10.03 Hz, 1H), 7.45 (d, J=7.95 Hz, 1H), 7.39-7.44 (m, 2H), 7.32 (t, J=7.70 Hz, 1H), 7.01 (s, 1H), 6.86 (d, J=7.70 Hz, 1H), 6.32 (s, 2H), 4.93 (d, J=6.11 Hz, 2H), 4.88 (d, J=6.11 Hz, 2H), 3.49 (s, 2H), 3.41 (td, J=6.48, 2.57 Hz, 2H), 2.97 (t, J=6.48 Hz, 2H), 2.84 (s, 3H); LCMS [M+H]⁺ 485.26.

### Example 586: 6-((6-fluoro-5-(4-(4-isopropylpiperazin-1-yl)phenyl)pyridin-2-yl)amino)-3,4-dihydroisoquinolin-1(2H)-one (I-639)

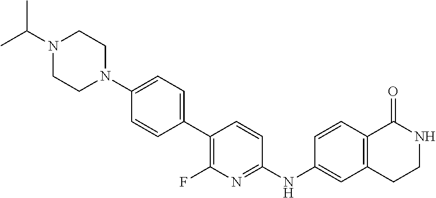 [SMILES: CC(C)N1CCN(c2ccc(-c3ccc(Nc4ccc5c(c4)CCNC5=O)nc3F)cc2)CC1]

Step 1: 6-((5-bromo-6-fluoropyridin-2-yl)amino)-3,4-dihydroisoquinolin-1(2H)-one

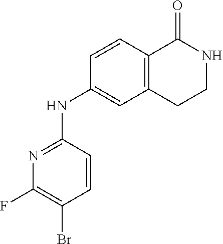 [SMILES: O=C1NCCc2cc(Nc3ccc(Br)c(F)n3)ccc21]

To a vial containing 6-bromo-3,4-dihydro-2h-isoquinolin-1-one (100 mg, 0.442 mmol), 6-amino-3-bromo-2-fluoropyridine (93 mg, 0.487 mmol), tris(dibenzylideneacetone)dipalladium (0) (20.25 mg, 0.022 mmol), Xantphos (25.6 mg, 0.044 mmol), and potassium phosphate tribasic (235 mg, 1.106 mmol) was added 1,4-dioxane (6 ml). The system was flushed with nitrogen and heated at 100° C. overnight. The reaction was concentrated onto celite and purified by flash chromatography (silica gel) eluting with 0-100% EtOAc/Hexanes. The desired fractions were collected, concentrated and dried under vacuum to afford 6-((5-bromo-6-fluoropyridin-2-yl)amino)-3,4-dihydroisoquinolin-1(2H)-one (131 mg, 0.390 mmol, 88% yield) as a yellow solid. LCMS [M+H]⁺ 336.08.

Step 2: 6-((6-fluoro-5-(4-(4-isopropylpiperazin-1-yl)phenyl)pyridin-2-yl)amino)-3,4-dihydroisoquinolin-1(2H)-one, hydrochloride

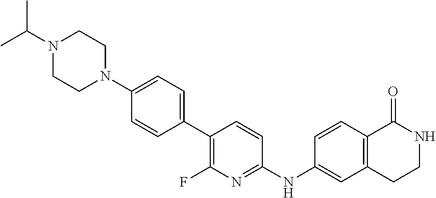 [SMILES: CC(C)N1CCN(c2ccc(-c3ccc(Nc4ccc5c(c4)CCNC5=O)nc3F)cc2)CC1]

The free base of the product was prepared from 6-((5-bromo-6-fluoropyridin-2-yl)amino)-3,4-dihydroisoquinolin-1(2H)-one (45 mg, 0.134 mmol) and 4-(4-isopropylpiperazinyl)phenylboronic acid, pinacol ester (53.1 mg, 0.161 mmol) to give 6-((6-fluoro-5-(4-(4-isopropylpiperazin-1-yl)phenyl)pyridin-2-yl)amino)-3,4-dihydroisoquinolin-1(2H)-one (18.5 mg, 30.1% yield) as a white solid. To a suspension of 6-((6-fluoro-5-(4-(4-isopropylpiperazin-1-yl)phenyl)pyridin-2-yl)amino)-3,4-dihydroisoquinolin-1(2H)-one (18.5 mg, 0.040 mmol) in THF (1 ml) was added hydrochloric acid, 4.0 M in dioxane (0.067 ml, 0.268 mmol). The reaction turned to a solution immediately and was stirred at room temperature for 1 hour. The reaction was concentrated in vacuo and dried on the lyophilizer to afford product as the hydrochloride salt (15.7 mg, 23.6% yield) as a pale-yellow solid. ¹H NMR (500 MHz, DMSO-d₆) δ ppm 10.84 (br. s., 1H), 9.83 (br. s., 1H), 7.86-7.94 (m, 1H), 7.77 (d, J=8.80 Hz, 1H), 7.67 (br. s., 1H), 7.56-7.63 (m, 2H), 7.47 (d, J=7.95 Hz, 2H), 7.09 (d, J=8.44 Hz, 2H), 6.94 (d, J=8.07 Hz, 1H), 3.90 (d, J=12.35 Hz, 2H), 3.50 (m, J=11.00 Hz, 3H), 3.36 (br. s., 2H), 3.20-3.29 (m, 2H), 3.10-3.19 (m, 2H), 2.87 (t, J=6.05 Hz, 2H), 1.33 (d, J=6.48 Hz, 6H); LCMS [M+H]⁺=460.06.

### Example 587: 6-(2-amino-5-(4-(4-((2,2-difluorocyclopropyl)methyl)piperazin-1-yl)phenyl)-6-fluoropyridin-3-yl)-3,4-dihydroisoquinolin-1(2H)-one (I-640)

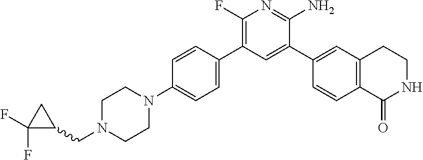 [SMILES: Nc1nc(F)c(-c2ccc(N3CCN(CC4CC4(F)F)CC3)cc2)cc1-c1ccc2c(c1)CCNC2=O]

Step 1: tert-butyl 4-(4-(6-amino-2-fluoro-5-(1-oxo-1,2,3,4-tetrahydroisoquinolin-6-yl)pyridin-3-yl)phenyl)piperazine-1-carboxylate

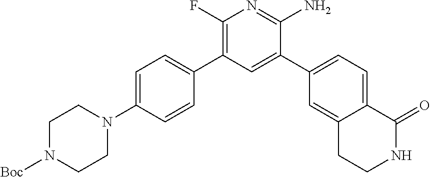 [SMILES: CC(C)(C)OC(=O)N1CCN(c2ccc(-c3cc(-c4ccc5c(c4)CCNC5=O)c(N)nc3F)cc2)CC1]

Prepared from 6-(2-amino-5-bromo-6-fluoropyridin-3-yl)-3,4-dihydroisoquinolin-1(2H)-one (700 mg, 2.082 mmol) and tert-butyl 4-[4-(4,4,5,5-tetramethyl-1,3,2-dioxaborolan-2-yl)phenyl]tetrahydro-1(2H)-pyrazinecarboxylate (1011 mg, 2.60 mmol) to afford the product as a beige solid (676 mg, yield 61%) LCMS: [M+H]⁺=518.47

Step 2: 6-(2-amino-6-fluoro-5-(4-(piperazin-1-yl)phenyl)pyridin-3-yl)-3,4-dihydroisoquinolin-1(2H)-one, Trifluoroacetic Acid salt

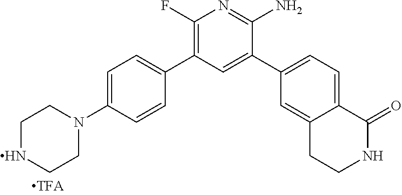

To a solution of the tert-butyl 4-(4-(6-amino-2-fluoro-5-(1-oxo-1,2,3,4-tetrahydroisoquinolin-6-yl)pyridin-3-yl)phenyl)piperazine-1-carboxylate (676 mg, 1.306 mmoln DCM (2 ml), was added TFA (9.00 ml, 118 mmol) at RT and the reaction mixture was continuously stirred at RT for 1.5 h The reaction mixture was concentrated, and triturated from Methanol to yield the title compound as a beige solid (636 mg, 92% yield). LCMS: [M+H]⁺=418.26.

Step 3: Preparation of 6-(2-amino-5-(4-(4-((2,2-difluorocyclopropyl)methyl)piperazin-1-yl)phenyl)-6-fluoropyridin-3-yl)-3,4-dihydroisoquinolin-1(2H)-one

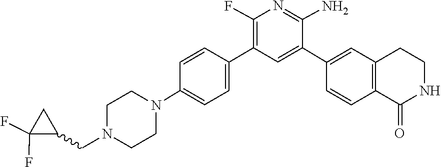 [SMILES: Nc1nc(F)c(-c2ccc(N3CCN(CC4CC4(F)F)CC3)cc2)cc1-c1ccc2c(c1)CCNC2=O]

To a slurry of the product of the foregoing procedure (75 mg, 0.141 mmol) in DMF (1.5 mL) at RT, was added N,N-diisopropylethylamine (0.246 ml, 1.411 mmol). After 5 min of stirring, 1-bromomethyl-2,2-difluorocyclopropane (72.4 mg, 0.423 mmol) was added and the reaction mixture was stirred at 60° C. for 5 h. The reaction mixture was partitioned between water (6 mL) and DCM (3 mL), the organic phase was separated, aqueous phase was extracted with DCM (1×3 ml), the combined organic phase was dried over Na₂SO₄ and concentrated. It was purified by silica gel chromatography, eluting with dichloromethane containing 0-4% Methanol and 0-0.4% NH₄OH to afford the title compound as a beige solid (49 mg, 65%). ¹H NMR (500 MHz, DMSO-d6) δ ppm 7.83-8.01 (m, 2H), 7.58 (d, J=10.15 Hz, 1H), 7.46 (d, J=8.07 Hz, 1H), 7.44 (s, 1H), 7.40 (d, J=8.07 Hz, 2H), 6.98 (d, J=8.68 Hz, 2H), 6.09-6.26 (m, 2H), 3.52-3.62 (m, 2H), 3.12-3.19 (m, 4H), 3.18 (br. s., 4H), 2.95 (t, J=6.36 Hz, 2H), 2.53-2.69 (m, 5H), 2.53-2.69 (m, 5H), 2.36 (dd, J=12.78, 8.13 Hz, 1H), 1.79-1.95 (m, 1H), 1.60 (tdd, J=11.97, 11.97, 7.67, 4.34 Hz, 1H), 1.16-1.26 (m, 1H); LCMS: [M+H]⁺=508.45

### Example 588: 6-(2-amino-5-(4-(4-(4,4-difluorobutyl)piperazin-1-yl)phenyl)-6-fluoropyridin-3-yl)-3,4-dihydroisoquinolin-1(2H)-one (I-641)

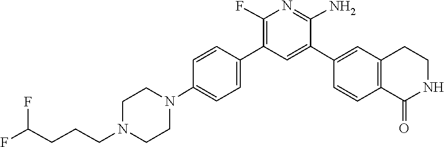 [SMILES: Nc1nc(F)c(-c2ccc(N3CCN(CCCC(F)F)CC3)cc2)cc1-c1ccc2c(c1)CCNC2=O]

Prepared as per the procedure analogous to that of Example 86, step 3, by heating the intermediate of 6-(2-amino-6-fluoro-5-(4-(piperazin-1-yl)phenyl)pyridin-3-yl)-3,4-dihydroisoquinolin-1(2H)-one, trifluoroacetic acid salt, (75 mg, 0.141 mmol) from Example 86, step 2 and N,N-diisopropylethylamine (0.246 ml, 1.411 mmol) to afford the title compound as a white solid (43 mg, 57% yield). ¹H NMR (500 MHz, DMSO-d₆) δ ppm 7.83-8.00 (m, 2H), 7.59 (d, J=10.15 Hz, 1H), 7.44-7.50 (m, 2H), 7.41 (d, J=8.19 Hz, 2H), 6.98 (d, J=8.56 Hz, 2H), 5.98-6.27 (m, 3H), 5.98-6.27 (m, 3H), 5.98-6.27 (m, 3H), 3.41 (d, J=4.03 Hz, 2H), 3.17 (br. s., 4H), 2.96 (t, J=6.30 Hz, 2H), 2.52-2.59 (m, 4H), 2.37 (d, J=6.48 Hz, 2 H), 1.79-1.94 (m, 2H), 1.59 (quin, J=7.31 Hz, 2H); LCMS: [M+H]⁺=510.39 Example 589: (S)-6-(2-amino-6-fluoro-5-(3-(1-methylpyrrolidin-2-yl)-4-(tetrahydro-2H-pyran-4-yl)phenyl)pyridin-3-yl)-3,4-dihydroisoquinolin-1(2H)-one (I-642)

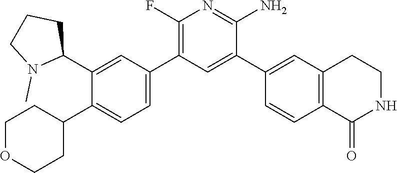 [SMILES: CN1CCC[C@H]1c1cc(-c2cc(-c3ccc4c(c3)CCNC4=O)c(N)nc2F)ccc1C1CCOCC1]

Step 1: (S)-2-(5-bromo-2-(tetrahydro-2H-pyran-4-yl)phenyl)-1-methylpyrrolidine

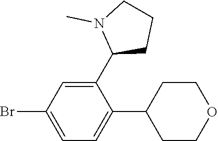 [SMILES: CN1CCC[C@H]1c1cc(Br)ccc1C1CCOCC1]

tert-Butyl (S)-2-(5-bromo-2-(tetrahydro-2H-pyran-4-yl)phenyl)pyrrolidine-1-carboxylate (100 mg, 0.244 mmol) (prepared as per patent WO 2020/193511, PCT/EP2020/058081) was deprotected as described in earlier examples to isolate (S)-2-(5-bromo-2-(tetrahydro-2H-pyran-4-yl)phenyl)pyrrolidine (83 mg, 99% yield) as a beige solid which was taken up in acetonitrile (3 mL) and Iodomethane (0.02 ml, 0.253 mmol) was added at 0-5° C. The reaction mixture was stirred overnight at RT, concentrated, and partitioned between water (4 ml) and dichloromethane (3 mL). The organic phase was separated, the aqueous phase was extracted with dichloromethane (3 mL), the combined org phase was washed with brine, dried over Na2SO4 and concentrated to yield the title compound as a beige solid (66 mg, 72% yield, 85% purity). LCMS: [M+H]⁺=324.29.

Step 2: (S)-6-(2-amino-6-fluoro-5-(3-(1-methylpyrrolidin-2-yl)-4-(tetrahydro-2H-pyran-4-yl)phenyl)pyridin-3-yl)-3,4-dihydroisoquinolin-1(2H)-one

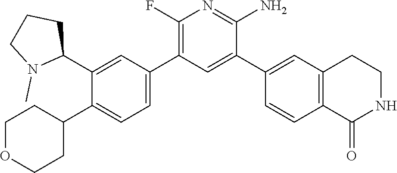 [SMILES: CN1CCC[C@H]1c1cc(-c2cc(-c3ccc4c(c3)CCNC4=O)c(N)nc2F)ccc1C1CCOCC1]

Prepared from (S)-2-(5-bromo-2-(tetrahydro-2H-pyran-4-yl)phenyl)-1-methylpyrrolidine 6-(2-amino-6-fluoro-5-(4,4,5,5-tetramethyl-1,3,2-dioxaborolan-2-yl)pyridin-3-yl)-3,4-dihydroisoquinolin-1(2H)-one and 6-(2-amino-5-bromo-6-fluoropyridin-3-yl)-3,4-dihydroisoquinolin-1(2H)-one to yield the title compound as a beige solid (54 mg, 59% yield with >95% purity). ¹H NMR (500 MHz, Methanol-d4) δ 7.98-7.91 (m, 1H), 7.70-7.64 (m, 1H), 7.62 (s, 1H), 7.59-7.54 (m, 1H), 7.48-7.43 (m, 1H), 7.43-7.40 (m, 1H), 7.39 (s, 1H), 4.85-4.78 (m, 1H), 4.03-3.92 (m, 2H), 3.82-3.70 (m, 1H), 3.62-3.53 (m, 2H), 3.49-3.42 (m, 2H), 3.34-3.25 (m, 1H), 3.13-3.04 (m, 1H), 3.00-2.92 (m, 2H), 2.76-2.63 (m, 3H), 2.54-2.45 (m, 1H), 2.31-2.07 (m, 3H), 1.98-1.86 (m, 1H), 1.78-1.67 (m, 1H), 1.66-1.60 (m, 1H), 1.58-1.49 (m, 1H); LCMS: [M+H]⁺=501.36.

### Example 590: (S)-7-(2-amino-6-fluoro-5-(3-(pyrrolidin-2-yl)-4-(tetrahydro-2H-pyran-4-yl)phenyl)pyridin-3-yl)-2-methylquinazolin-4(3H)-one (I-643)

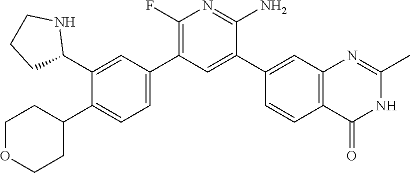 [SMILES: Cc1nc2cc(-c3cc(-c4ccc(C5CCOCC5)c([C@@H]5CCCN5)c4)c(F)nc3N)ccc2c(=O)[nH]1]

Prepared from 7-(2-amino-6-fluoro-5-(4,4,5,5-tetramethyl-1,3,2-dioxaborolan-2-yl)pyridin-3-yl)-2-methylquinazolin-4(3H)-one and tert-butyl (S)-2-(5-bromo-2-(tetrahydro-2H-pyran-4-yl)phenyl)pyrrolidine-1-carboxylate (prepared as per patent WO 2020/193511, PCT/EP2020/058081) (52 mg, 0.126 mmol) followed by deprotection to give the title compound as an off white solid (21 mg, 69% yield with >95% purity). ¹H NMR (500 MHz, Methanol-d4) δ 8.21-8.13 (m, 1H), 7.69-7.66 (m, 1H), 7.66-7.62 (m, 1H), 7.56-7.51 (m, 2H), 7.39-7.35 (m, 1H), 7.30-7.26 (m, 1H), 4.47-4.40 (m, 1H), 3.99-3.93 (m, 2H), 3.56-3.49 (m, 2H), 3.18-3.13 (m, 1H), 3.13-3.05 (m, 1H), 2.99-2.90 (m, 1H), 2.39-2.34 (m, 3H), 2.23-2.14 (m, 1H), 1.98-1.84 (m, 2H), 1.82-1.75 (m, 2H), 1.69-1.61 (m, 2H), 1.60-1.55 (m, 1H). LCMS: [M+H]⁺=500.30.

### Example 591: 6-(2-amino-5-(4-(4-(2-(2,2-difluorocyclopropyl)ethyl)piperazin-1-yl)phenyl)-6-fluoropyridin-3-yl)-3,4-dihydroisoquinolin-1(2H)-one(1-644)

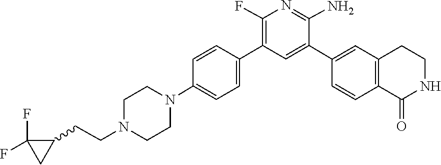 [SMILES: Nc1nc(F)c(-c2ccc(N3CCN(CCC4CC4(F)F)CC3)cc2)cc1-c1ccc2c(c1)CCNC2=O]

Prepared from 6-(2-amino-6-fluoro-5-(4-(piperazin-1-yl)phenyl)pyridin-3-yl)-3,4-dihydroisoquinolin-1(2H)-one TFA salt (75 mg, 0.141 mmol) and 2-(2-bromoethyl)-1,1-difluorocyclopropane (78 mg, 0.423 mmol) to afford the title compound as a beige solid (44 mg, 59% yield with >95% purity). 1H NMR (500 MHz, DMSO-d6) δ ppm 7.85-8.02 (m, 2 H), 7.55-7.65 (m, 1H), 7.34-7.52 (m, 4H), 6.98 (d, J=8.68 Hz, 2H), 6.17 (s, 2H), 3.42-3.46 (m, 2H), 3.13-3.19 (m, 4H), 2.96 (t, J=6.30 Hz, 2H), 2.55 (br. s., 4H), 2.42 (t, J=6.91 Hz, 2H), 1.63-1.76 (m, 2H), 1.47-1.62 (m, 2H), 1.13 (ddt, J=9.92, 6.74, 3.53, 3.53 Hz, 1 H); LCMS: [M+H]⁺=522.49.

### Example 592: 6-(2-amino-5-(3-((dimethylamino)methyl)-4-(tetrahydro-2H-pyran-4-yl)phenyl)-6-fluoropyridin-3-yl)-3,4-dihydroisoquinolin-1(2H)-one (I-645)

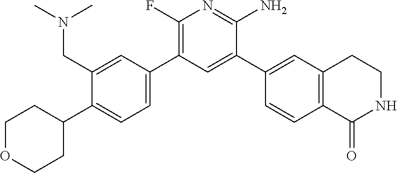 [SMILES: CN(C)Cc1cc(-c2cc(-c3ccc4c(c3)CCNC4=O)c(N)nc2F)ccc1C1CCOCC1]

Prepared from 1-(5-bromo-2-(tetrahydro-2H-pyran-4-yl)phenyl)-N,N-dimethylmethanamine (60 mg, 0.157 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (78 mg, 0.204 mmol) to yield the title compound as an off white solid (28 mg, 37% yield). ¹H NMR (500 MHz, METHANOL-d₄) δ ppm 8.06 (d, J=7.95 Hz, 1H), 7.69 (d, J=9.78 Hz, 1H), 7.53 (dd, J=8.01, 1.53 Hz, 1H), 7.44-7.50 (m, 3H), 7.39 (d, J=8.19 Hz, 1H), 4.04-4.13 (m, 2H), 3.51-3.66 (m, 7H), 3.08 (t, J=6.60 Hz, 2H), 2.29 (s, 6H), 1.83-1.93 (m, 2H), 1.73 (d, J=12.47 Hz, 2 H)LCMS: [M+H]⁺=475.35.

### Example 593: (S)-6-(2-amino-6-fluoro-5-(3-(pyrrolidin-2-yl)phenyl)pyridin-3-yl)-3,4-dihydroisoquinolin-1(2H)-one (I-646)

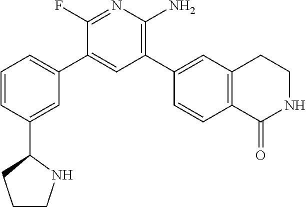 [SMILES: Nc1nc(F)c(-c2cccc([C@@H]3CCCN3)c2)cc1-c1ccc2c(c1)CCNC2=O]

Prepared from (2S)-2-(3-bromophenyl)pyrrolidine (39 mg, 0.172 mmol) and 6-(2-amino-6-fluoro-5-(4,4,5,5-tetramethyl-1,3,2-dioxaborolan-2-yl)pyridin-3-yl)-3,4-dihydroisoquinolin-1(2H)-one (83 mg, 0.216 mmol) to give the title compound as a beige solid. (55 mg, 75% yield). ¹H NMR (500 MHz, Methanol-d₄) δ ppm 8.09-8.03 (m, 1H), 7.73-7.67 (m, 1H), 7.57-7.51 (m, 2H), 7.49-7.43 (m, 2H), 7.42-7.37 (m, 1H), 7.36-7.32 (m, 1H), 4.13-4.07 (m, 1H), 3.60-3.55 (m, 2H), 3.24-3.17 (m, 1H), 3.11-3.05 (m, 2H), 3.01-2.93 (m, 1H), 2.32-2.22 (m, 1H), 2.05-1.97 (m, 1H), 1.96-1.87 (m, 1H), 1.82-1.72 (m, 1H); LCMS: [M+H]⁺=403.40.

### B. Preparation of Comparative Compounds

### Example C-1: 6-(2-amino-5-(4-(piperidin-4-yl)phenyl)pyridin-3-yl)-3,4-dihydroisoquinolin-1(2H)-one, Comparator 1

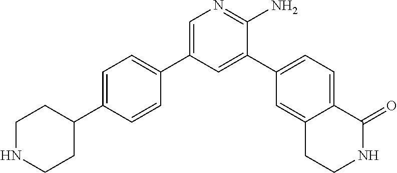 [SMILES: Nc1ncc(-c2ccc(C3CCNCC3)cc2)cc1-c1ccc2c(c1)CCNC2=O]

Step 1: 6-(4,4,5,5-tetramethyl-1,3,2-dioxaborolan-2-yl)-3,4-dihydroisoquinolin-1(2H)-one

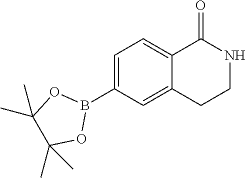 [SMILES: CC1(C)OB(c2ccc3c(c2)CCNC3=O)OC1(C)C]

To a degassed suspension of 6-bromo-3,4-dihydroisoquinolin-1(2H)-one (3 g, 13.3 mmol), 4,4,4′,4′,5,5,5′,5′-octamethyl-2,2′-bi(1,3,2-dioxaborolane) (5.07 g, 20 mmol), KOAc (3.92 g, 40 mmol) in 1,4-dioxane (30 mL) was added a Pd(dppf)Cl₂-DCM (1.08 g, 1.3 mmol). The reaction mixture was heated at 85° C. for 16 h under argon atmosphere before cooling to RT. The reaction mixture was filtered through a Celite® bed, which was washed in with ethyl acetate (100 mL). The combined filtrate was concentrated under reduced pressure and the residue obtained was purified by column chromatography (silica gel 100-200 mesh), eluting with 50-70% EtOAc in pet ether to afford the product (3 g, 82%) as a pale yellow solid.

Step 2. 6-(2-amino-5-bromopyridin-3-yl)-3,4-dihydroisoquinolin-1(2H)-one

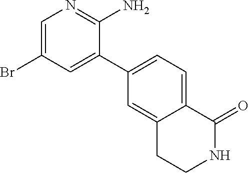 [SMILES: Nc1ncc(Br)cc1-c1ccc2c(c1)CCNC2=O]

To a degassed suspension of 6-(4,4,5,5-tetramethyl-1,3,2-dioxaborolan-2-yl)-3,4-dihydroisoquinolin-1(2H)-one (2.5 g, 9.1 mmol) 5-bromo-3-iodopyridin-2-amine (2.72 g, 9.1 mmol) and K₂CO₃ (2.52 g, 18.3 mmol) in DMF: H₂O (40:5 mL) was added Pd(PPh₃)₂Cl₂ (0.64 g, 0.9 mmol) at RT−65° C. over 16 h before cooling to RT. The reaction mixture was poured into ice water (100 mL) extracted with CH₂Cl₂ (2×100 mL) washed with water (50 mL), followed by brine (50 mL). The separated organic layer was dried over Na₂SO₄ and concentrated under reduced pressure and the residue obtained was purified by column chromatography (silica gel 100-200 mesh), eluting with 0-10% MeOH in DCM to afford the product (1.3 g, 44%) as a pale yellow solid. LCMS: [M+H]⁺=318.18.

Step 3: tert-butyl 4-(4-(6-amino-5-(1-oxo-1,2,3,4-tetrahydroisoquinolin-6-yl)pyridin-3-yl)phenyl)piperidine-1-carboxylate

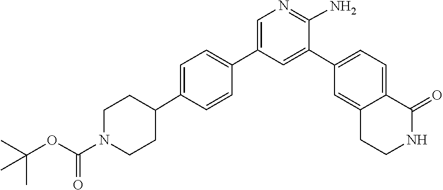 [SMILES: CC(C)(C)OC(=O)N1CCC(c2ccc(-c3cnc(N)c(-c4ccc5c(c4)CCNC5=O)c3)cc2)CC1]

A mixture of 6-(2-amino-5-bromopyridin-3-yl)-3,4-dihydroisoquinolin-1(2H)-one (125 mg, 0.393 mmol), Cs₂CO₃ (384 mg, 1.18 mmol), 4-(N-Boc-piperidino)phenylboronic acid pinacol ester (183 mg, 0.471 mmol) and [1,1′-bis(diphenylphosphino)ferrocene]dichloropalladium(II) (34.5 mg, 0.047 mmol) in DME (4.0 mL) and H₂O (0.800 mL) in a microwave vial was flushed with argon. The mixture was heated in a microwave reactor at 80° C. for 2 h. The reaction mixture was partitioned between EtOAc and brine, the organic phase was separated and the aqueous phase was extracted with CH₂Cl₂. The combined organic layers were dried over Na₂SO₄ and concentrated onto Celite®. Purification by silica gel chromatography (4 g), eluting with CH₂Cl₂ containing 0-2% MeOH and 0-0.2% NH₄OH afforded the product as a beige solid (137 mg, 70%). LCMS: [M +H]⁺=499.71

Step 4: 6-(2-amino-5-(4-(piperidin-4-yl)phenyl)pyridin-3-yl)-3,4-dihydroisoquinolin-1(2H)-one

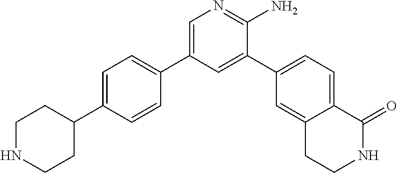 [SMILES: Nc1ncc(-c2ccc(C3CCNCC3)cc2)cc1-c1ccc2c(c1)CCNC2=O]

To a slurry of tert-butyl 4-(4-(6-amino-5-(1-oxo-1,2,3,4-tetrahydroisoquinolin-6-yl)pyridin-3-yl)phenyl)piperidine-1-carboxylate (137 mg, 0.275 mmol) in CH₂Cl₂ (3 mL) was added HCl in dioxane (3.43 mL of a 4.0 N solution, 13.74 mmol) and the mixture was stirred at RT for 1 h. The mixture was concentrated to dryness, dissolved in MeOH, passed through a porapak cartridge to collect, after removing the solvent, the free base as an off-white solid (101 mg, 88%)¹H NMR (500 MHz, DMSO-d₆) δ 8.34-8.24 (m, 1H), 7.99-7.90 (m, 2H), 7.67-7.61 (m, 1H), 7.60-7.54 (m, 2H), 7.53-7.46 (m, 2H), 7.31-7.24 (m, 2H), 5.83 (s, 2H), 3.45-3.40 (m, 2H), 3.08-3.00 (m, 2H), 2.99-2.94 (m, 2H), 2.65-2.56 (m, 3H), 1.74-1.66 (m, 2H), 1.58-1.48 (m, 2H). LCMS: [M+H]⁺=399.63.

### Example C-2: 6-(2-amino-5-(1-(tetrahydro-2H-pyran-4-yl)-1H-pyrazol-4-yl)pyridin-3-yl)-3,4-dihydroisoquinolin-1(2H)-one, Comparator 2

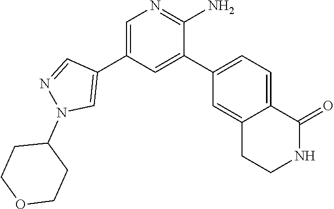 [SMILES: Nc1ncc(-c2cnn(C3CCOCC3)c2)cc1-c1ccc2c(c1)CCNC2=O]

Step 1: 5-bromo-3-chloropyridin-2-amine

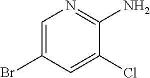

To a solution of 3-chloropyridin-2-amine (4.0 g, 31.1 mmol) in CH₃CN (100 mL) was added N-bromosuccinimide (5.54 g, 31.1 mmol) in several portions at 0° C. The reaction was allowed to warm slowly and stirred at RT overnight. The reaction mixture was stored at −20° C. for 5 d. Light orange crystals (plates) that formed were collected and rinsed with small amount of MeCN to afford the product (5.6 g, 87%) as a light orange solid. ¹H NMR (500 MHz, CDCl₃) δ 8.40-8.74 (m, 0.5H), 8.03 (d, J=2.1 Hz, 1H), 7.63 (d, J=2.1 Hz, 1H), 4.98 (br s, 2H), 2.78 (s, 2H).

Step 2: 3-chloro-5-(1-(tetrahydro-2H-pyran-4-yl)-1H-pyrazol-4-yl)pyridin-2-amine

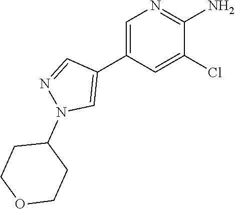 [SMILES: Nc1ncc(-c2cnn(C3CCOCC3)c2)cc1Cl]

A vial charged with 5-bromo-3-chloropyridin-2-amine (100 mg, 0.482 mmol), Cs₂CO₃ (471 mg, 1.45 mmol), 1-(tetrahydro-pyran-4-yl)-1H-pyrazole-4-boronic acid pinacol ester (147 mg, 0.530 mmol) and [1,1′-bis(diphenylphosphino)ferrocene]dichloropalladium(II) (35.4 mg, 0.048 mmol) was suspended in water (2 mL). The reaction was degassed, and DME (4 mL) was added. The reaction was degassed again by evacuation-refill with argon then heated sealed under microwave irradiation at 100° C. for 3 h. The reaction mixture was concentrated under reduced pressure, deposited on Celite® and purified by flash silica gel chromatography, eluting with 8% MeOH in CH₂Cl₂, to afford the product as a tan solid (95 mg, 71%). LCMS: [M+H]⁺=278.99.

Step 3: 6-(2-amino-5-(1-(tetrahydro-2H-pyran-4-yl)-1H-pyrazol-4-yl)pyridin-3-yl)-3,4-dihydroisoquinolin-1(2H)-one

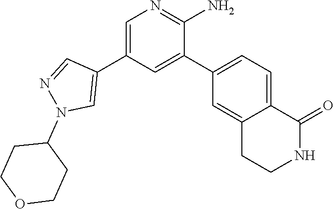 [SMILES: Nc1ncc(-c2cnn(C3CCOCC3)c2)cc1-c1ccc2c(c1)CCNC2=O]

To a mixture of 6-(4,4,5,5-tetramethyl-1,3,2-dioxaborolan-2-yl)-3,4-dihydroisoquinolin-1(2H)-one (37.6 mg, 0.138 mmol), 3-chloro-5-(1-(tetrahydro-2H-pyran-4-yl)-1H-pyrazol-4-yl)pyridin-2-amine (32 mg, 0.115 mmol), K₃PO₄ (97 mg, 0.459 mmol), palladium(II) acetate (2.58 mg, 0.011 mmol) and XPhos (13.68 mg, 0.029 mmol) under an atmosphere of argon before water (2 mL) and CH₃CN (3 mL) were added. The mixture was degassed with argon and heated in a microwave reactor at 100° C. for 2 h. The reaction mixture was concentrated under reduced pressure, deposited on Celite® and purified by preparative HPLC (30 g Biotage® SNAP KP-C18-HS, MeOH in (H₂O+0.05% TFA)) eluting at 30% MeOH, pooled fractions: 17-25 to afford the TFA salt of the title compound as a white solid (19.4 mg, 33%). LCMS: [M+H]⁺=390.43. ¹H NMR (500 MHz, MeOD-d₄) δ 8.21 (s, 1H), 8.17 (br d, J=9.7 Hz, 2H), 8.11 (d, J=8.0 Hz, 1H), 7.93 (s, 1H), 7.54 (br d, J=8.0 Hz, 1H), 7.50 (s, 1H), 4.39-4.50 (m, 1H), 4.04-4.10 (m, 2H), 3.52-3.63 (m, 4H), 3.05-3.12 (m, 2H), 2.03-2.14 (m, 4H).

### Example C-3: 6-(2-amino-5-(4-(pyrrolidin-1-yl)phenyl)pyridin-3-yl)-3,4-dihydroisoquinolin-1(2H)-one, Comparator 3

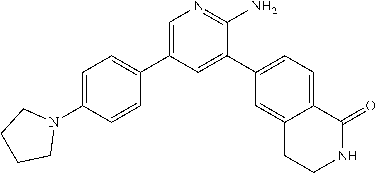 [SMILES: Nc1ncc(-c2ccc(N3CCCC3)cc2)cc1-c1ccc2c(c1)CCNC2=O]

Step 1: 3-chloro-5-(4-(pyrrolidin-1-yl)phenyl)pyridin-2-amine

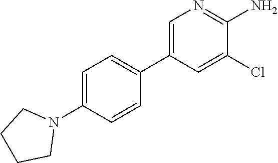 [SMILES: Nc1ncc(-c2ccc(N3CCCC3)cc2)cc1Cl]

A 30 mL vial was charged with 5-bromo-3-chloropyridin-2-amine (0.047 g, 0.228 mmol), 1-(4-(4,4,5,5-tetramethyl-1,3,2-dioxaborolan-2-yl)phenyl)pyrrolidine (0.052 g, 0.190 mmol), [1,1′-bis(diphenylphosphino)ferrocene]dichloropalladium(II) (0.014 g, 0.019 mmol) and Cs₂CO₃ (0.155 g, 0.476 mmol). After the vial was sealed with a cap and septum the reaction vessel was evacuated and backfilled with nitrogen. 1,4-Dioxane (2 mL) and water (1 mL) were added via syringe and the vessel was evacuated and backfilled with nitrogen an additional time. The reaction was heated to 80° C. for 16 h, concentrated onto Celite® and purified by flash silica gel chromatography (0.5-10% MeOH/DCM+0.5% NH₄OH) to afford the product (45 mg, 86% yield). LCMS: [M+H]⁺=317.3.

Step 2: 6-(2-amino-5-(4-(pyrrolidin-1-yl)phenyl)pyridin-3-yl)-3,4-dihydroisoquinolin-1(2H)-one

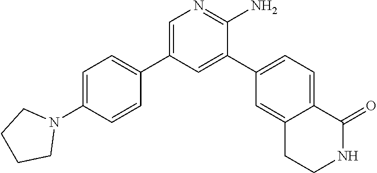 [SMILES: Nc1ncc(-c2ccc(N3CCCC3)cc2)cc1-c1ccc2c(c1)CCNC2=O]

A 30 mL vial with magnetic stir bar was charged 3-chloro-5-(4-(pyrrolidin-1-yl)phenyl)pyridin-2-amine (0.0225 g, 0.082 mmol), 3,4-dihydro-1(2H)-isoquinolinone-6-boronic acid pinacol ester (0.027 g, 0.099 mmol) and XPhos Pd G2 (6.47 mg, 8.22 μmol). The vial was sealed with a cap and septum and the reaction vessel was evacuated and backfilled with nitrogen. Butan-1-ol (1 mL) and aqueous K₃PO₄ (0.158 mL, 0.205 mmol) were added and the reaction vessel was evacuated and backfilled with nitrogen an additional time. The reaction mixture was heated at 90° C. for 18 h in an aluminum block, allowed to cool to RT and concentrated onto Celite. Purification by flash chromatography (eluting with 0.5-10% MeOH/DCM+0.5% NH₄OH) followed by reverse phase flash chromatography (eluting with 5-60% MeCN/water+0.1% formic acid) afforded the title compound (15 mg, 48%) as a yellow solid. ¹H NMR (500 MHz, DMSO-d₆) δ 8.22 (d, J=2.3 Hz, 1H), 7.9-8.0 (m, 2H), 7.55 (d, J=2.3 Hz, 1H), 7.4-7.5 (m, 4H), 6.58 (br d, J=8.7 Hz, 2H), 5.65 (s, 2H), 3.4-3.4 (m, 2H), 3.24 (br t, J=6.4 Hz, 4H), 2.96 (br t, J=6.4 Hz, 2H), 1.9-2.0 (m, 4H). LCMS: [M+H]⁺=385.26.

### Example C-4: 5-(2-amino-5-(4-(piperidin-1-yl)phenyl)pyridin-3-yl)isoindolin-1-one, Comparator 4

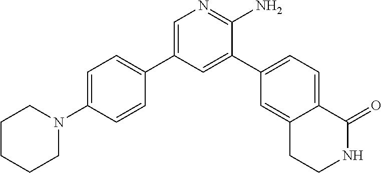 [SMILES: Nc1ncc(-c2ccc(N3CCCCC3)cc2)cc1-c1ccc2c(c1)CCNC2=O]

Step 1: 1-(4-chlorophenyl)piperidine

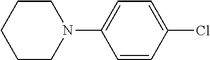 [SMILES: Clc1ccc(N2CCCCC2)cc1]

A 30 mL vial was charged with 1-chloro-4-iodobenzene (0.250 g, 1.048 mmol), tris(dibenzylideneacetone)dipalladium (0) (0.029 g, 0.031 mmol), 4,5-bis(diphenylphosphino)-9,9-dimethylxanthene (0.055 g, 0.094 mmol) and potassium tert-butoxide (0.353 g, 3.15 mmol). The vial was sealed with a cap and septum and then the reaction vessel was evacuated and backfilled with nitrogen. Toluene (5 mL) and piperidine (0.104 mL, 1.05 mmol) were added and the reaction vessel was evacuated and backfilled with nitrogen an additional time. The reaction mixture was heated at 100° C. for 18 h, allowed to cool to RT, loaded onto Celite® and purified by flash silica gel chromatography (1-15% EtOAc/hexanes) to afford the product (159 mg, 77%). LCMS: [M+H]⁺=196.27.

Step 2: 1-(4-(4,4,5,5-tetramethyl-1,3,2-dioxaborolan-2-yl)phenyl)piperidine

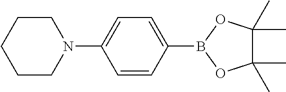 [SMILES: CC1(C)OB(c2ccc(N3CCCCC3)cc2)OC1(C)C]

A 30 mL vial was charged with 1-(4-chlorophenyl)piperidine (0.159 g, 0.813 mmol), bis(pinacolato)diboron (0.309 g, 1.22 mmol), KOAc (0.159 g, 1.63 mmol) and XPhos Pd G2 (0.048 g, 0.061 mmol). The vial was evacuated and backfilled with nitrogen. 1,4-Dioxane (5 mL) was added and the vial was evacuated and backfilled an additional time. The reaction mixture was then heated to 110° C. in an aluminum block for 18 h, concentrated onto Celite® and purified by flash (1-10% EtOAc/hexanes) to afford the product (140 mg, 60%). LCMS: [M+H]⁺=287.93.

Step 3: 3-chloro-5-(4-(piperidin-1-yl)phenyl)pyridin-2-amine

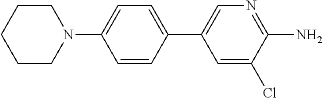 [SMILES: Nc1ncc(-c2ccc(N3CCCCC3)cc2)cc1Cl]

A 30 mL vial was charged with 5-bromo-3-chloropyridin-2-amine (0.121 g, 0.585 mmol), 1-(4-(4,4,5,5-tetramethyl-1,3,2-dioxaborolan-2-yl)phenyl)piperidine (0.14 g, 0.487 mmol), [1,1′-bis(diphenylphosphino)ferrocene]dichloropalladium(II) (0.036 g, 0.049 mmol) and Cs₂CO₃ (0.397 g, 1.22 mmol). After the vial was sealed with a cap and septum the reaction vessel was evacuated and backfilled with nitrogen. 1,4-Dioxane (4 mL) and water (2 mL) were added via syringe, the vessel was evacuated and backfilled with nitrogen an additional time. The reaction was heated to 80° C. overnight, concentrated onto Celite® and purified by flash chromatography (0.5-10% MeOH/DCM+0.5% NH₄OH) to afford the product (105 mg, 75%). LCMS: [M+H]⁺=288.01.

Step 4: 5-(2-amino-5-(4-(piperidin-1-yl)phenyl)pyridin-3-yl)isoindolin-1-one

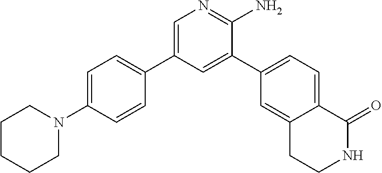 [SMILES: Nc1ncc(-c2ccc(N3CCCCC3)cc2)cc1-c1ccc2c(c1)CCNC2=O]

In a 30 mL vial with magnetic stir bar was charged 3-chloro-5-(4-(piperidin-1-yl)phenyl)pyridin-2-amine (0.050 g, 0.174 mmol), 3,4-dihydro-1(2H)-isoquinolinone-6-boronic acid pinacol ester (0.057 g, 0.208 mmol) and XPhos Pd G2 (0.014 g, 0.017 mmol). The vial was sealed with a cap and septum and the reaction vessel was evacuated and backfilled with nitrogen. Butan-1-ol (2 ml) and aqueous K₃PO₄ (0.334 mL of a 1.3 M solution, 0.434 mmol) were added and the reaction vessel was evacuated and backfilled with nitrogen an additional time. The reaction mixture was heated at 90° C. for 18 h in an aluminum block, concentrated onto Celite® and purified by flash silica gel chromatography (0.5-10% MeOH/DCM+0.5% NH₄OH) followed by reverse phase flash chromatography (5-60% MeCN/water+0.1% formic acid) to afford the title compound (24 mg, 35%) as a pale yellow solid. ¹H NMR (500 MHz, DMSO-d₆) δ 8.25 (d, 1H, J=2.3 Hz), 7.9-8.0 (m, 2H), 7.58 (d, 1H, J=2.3 Hz), 7.5-7.5 (m, 4H), 6.97 (br d, 2H, J=8.8 Hz), 5.73 (s, 2H), 3.4-3.4 (m, 2H), 3.1-3.2 (m, 4H), 2.97 (br t, J=6.3 Hz, 2H), 1.63 (br s, 4H), 1.55 (br d, J=4.4 Hz, 2H). LCMS: [M+H]⁺=399.18.

### Example C-5: 6-(2-amino-5-(4-((3aR,6aS)-tetrahydro-1H-furo[3,4-c]pyrrol-5(3H)-yl)phenyl)pyridin-3-yl)-3,4-dihydroisoquinolin-1(2H)-one, Comparator 5

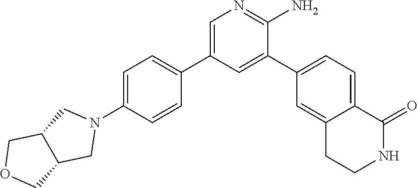 [SMILES: Nc1ncc(-c2ccc(N3C[C@H]4COC[C@H]4C3)cc2)cc1-c1ccc2c(c1)CCNC2=O]

Step 1: 6-(2-amino-5-(4,4,5,5-tetramethyl-1,3,2-dioxaborolan-2-yl)pyridin-3-yl)-3,4-dihydroisoquinolin-1(2H)-one

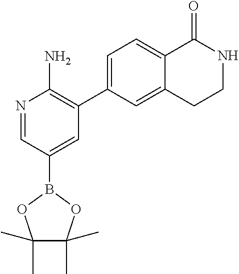 [SMILES: CC1(C)OB(c2cnc(N)c(-c3ccc4c(c3)CCNC4=O)c2)OC1(C)C]

To a degassed suspension of 6-(2-amino-5-bromopyridin-3-yl)-3,4-dihydroisoquinolin-1(2H)-one (2.5 g, 7.8 mmol, prepared according to the procedure described in Example 1), 4,4,4′,4′,5,5,5′,5′-octamethyl-2,2′-bi(1,3,2-dioxaborolane (3 g, 7.8 mmol), KOAc (2.32 g, 23.6 mmol) in 1,4-dioxane (40 mL) was added a Pd(dppf)Cl₂. CH₂Cl₂ (0.64 g, 0.7 mmol) and then, the reaction mixture was again degassed with argon for 10 min. The reaction mixture was heated to 85° C. for 16 h under argon atmosphere. The reaction mixture was filtered through a Celite® bed, which was washed with EtOAc (50 mL). The combined filtrate was concentrated under reduced pressure and the residue was purified by washed with n-pentane followed by diethyl ether to afford the product (2 g, 69%) as a brown solid. TLC system: 5% MeOH: CH₂Cl₂; R_{f}=0.3.

Step 2: (3aR,6aS)-5-(4-iodophenyl)hexahydro-1H-furo[3,4-c]pyrrole

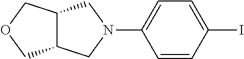 [SMILES: Ic1ccc(N2C[C@H]3COC[C@H]3C2)cc1]

A sealed mixture of hexahydro-1H-furo[3,4-c]pyrrole hydrochloride (0.564 g, 3.77 mmol) and K₃PO₄ (1.60 g, 7.54 mmol) in DMF (20 mL) was stirred at RT for 16 h. The mixture was then degassed with a stream of N₂ and then copper (I) iodide (0.144 g, 0.754 mmol), (R)-(+)-1,1′-bi-2-naphthol (0.216 g, 0.754 mmol) and 1,4-diiodobenzene (1.24 g, 3.77 mmol) were added as solids. Then degassing step was repeated. The mixture was then stirred sealed at 40° C. for 12 h. The reaction mixture was diluted with EtOAc and filtered through Celite®. The reaction mixture was concentrated under reduced pressure, and purified by flash silica gel chromatography, eluting with CH₂Cl₂, to afford the product as a yellow solid (659.0 mg, 50% based on purity of 90%); LCMS: [M+H]⁺=315.99.

Step 3: 6-(2-amino-5-(4-((3aR,6aS)-tetrahydro-1H-furo[3,4-c]pyrrol-5(3H)-yl)phenyl)pyridin-3-yl)-3,4-dihydroisoquinolin-1(2H)-one

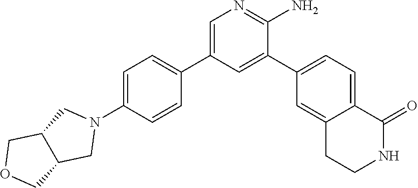 [SMILES: Nc1ncc(-c2ccc(N3C[C@H]4COC[C@H]4C3)cc2)cc1-c1ccc2c(c1)CCNC2=O]

(3aR,6aS)-5-(4-Iodophenyl)hexahydro-1H-furo[3,4-c]pyrrole (67.1 mg, 0.192 mmol), [1,1′-bis(diphenylphosphino)ferrocene]dichloropalladium(II) (14.02 mg, 0.019 mmol), 6-(2-amino-5-(4,4,5,5-tetramethyl-1,3,2-dioxaborolan-2-yl)pyridin-3-yl)-3,4-dihydroisoquinolin-1(2H)-one (70 mg, 0.192 mmol) and Cs₂CO₃ (187 mg, 0.575 mmol) were suspended in water (2 mL), degassed with N₂ and DME (4 mL) was added. Degassing was repeated and the reaction mix was heated under microwave irradiation at 90° C. for 1 h. The reaction was treated with 1 mL of 1 M aq HCl and filtered through a Waters PoraPak™ CX column (0.5 g), rinsing with MeOH and eluting with 2 M NH₃ in MeOH and then CH₂Cl₂. The reaction mixture was concentrated under reduced pressure and purified by flash chromatography (25 g SiO₂ Biotage® cartridge, using MeOH in CH₂Cl₂ eluting at 6% MeOH, to afford the title compound (23 mg 28%) as a light yellow solid. ¹H NMR (500 MHz, DMSO-d₆) δ 8.23 (d, J=2.5 Hz, 1H), 7.94 (br s, 1H), 7.92 (d, J=7.8 Hz, 1H), 7.56 (d, J=2.3 Hz, 1H), 7.43-7.52 (m, 4H), 6.68 (d, J=8.7 Hz, 2H), 5.68 (s, 2H), 3.85 (br dd, J=8.7, 6.7 Hz, 2H), 3.55 (dd, J=8.8, 3.6 Hz, 2H), 3.34-3.44 (m, 4H), 3.14-3.23 (m, 2H), 2.93-3.04 (m, 4 H); LCMS: [M+H]⁺=427.47.

### Example C-6: 6-(2-amino-5-(4-((3aS,6aS)-hexahydro-5H-furo[2,3-c]pyrrol-5-yl)phenyl)pyridin-3-yl)-3,4-dihydroisoquinolin-1(2H)-one, Comparator 6

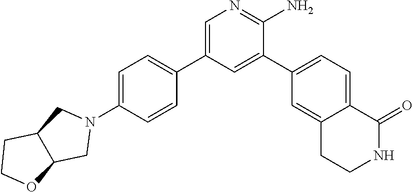 [SMILES: Nc1ncc(-c2ccc(N3C[C@@H]4CCO[C@@H]4C3)cc2)cc1-c1ccc2c(c1)CCNC2=O]

Step 1: (3aS,6aS)-5-(4-iodophenyl)hexahydro-2H-furo[2,3-c]pyrrole

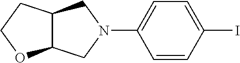 [SMILES: Ic1ccc(N2C[C@@H]3CCO[C@@H]3C2)cc1]

A sealed mixture of 1,4-diiodobenzene (2 g, 6.06 mmol), (3aS,6aS)-hexahydro-2H-furo[2,3-c]pyrrole hydrochloride (0.907 g, 6.06 mmol) and K₃PO₄ (2.57 g, 12.1 mmol) in DMF (20 mL) was stirred at RT overnight. The mixture was then degassed with a stream of N₂ and then copper (I) iodide (0.231 g, 1.21 mmol) and a (R)-(+)-1,1′-bi-2-naphthol (0.347 g, 1.21 mmol) were added as solids. Then degassing step was repeated. The mixture was then sealed, stirred at RT for 30 min and then at 40° C. for 4 d. The reaction mixture was concentrated under reduced pressure, and purified by flash silica gel chromatography (eluting with MeOH in CH₂Cl₂ eluting at 0-1% MeOH, to afford the product as a yellow-grey solid (940 mg, 48%) LCMS: [M+H]⁺=316.14.

Step 2: 6-(2-amino-5-(4-((3aS,6aS)-hexahydro-5H-furo[2,3-c]pyrrol-5-yl)phenyl)pyridin-3-yl)-3,4-dihydroisoquinolin-1(2H)-one

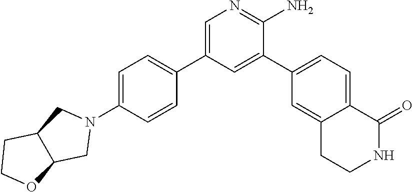 [SMILES: Nc1ncc(-c2ccc(N3C[C@@H]4CCO[C@@H]4C3)cc2)cc1-c1ccc2c(c1)CCNC2=O]

(3aS,6aS)-5-(4-Iodophenyl)hexahydro-2H-furo[2,3-c]pyrrole (62.3 mg, 0.192 mmol), [1,1′-bis(diphenylphosphino)ferrocene]dichloropalladium(II) (14.06 mg, 0.019 mmol), 6-(2-amino-5-(4,4,5,5-tetramethyl-1,3,2-dioxaborolan-2-yl)pyridin-3-yl)-3,4-dihydroisoquinolin-1(2H)-one (70 mg, 0.192 mmol) and Cs₂CO₃ (187 mg, 0.575 mmol) were suspended in water (2 mL). The mixture was degassed with N₂ and DME (4 mL) was added. Degassing was repeated and the mixture was heated under microwave irradiation at 90° C. for 1 h, concentrated under reduced pressure and purified by flash silica gel chromatography (eluting with eluting at 6% MeOH in CH₂Cl₂. The crude material was repurified by preparative HPLC (30 g Biotage® SNAP KP-C₁₈-HS, MeOH in (H₂O no acid)) eluting at 70% MeOH. The collected material was then filtered through a Isolute SCX-2 column (0.5 g), rinsing with MeOH and eluting with 2 M NH₃ in MeOH to afford the title compound as a light tan solid (13 mg, 15%). ¹H NMR (500 MHz, CDCl₃) δ 8.23 (d, J=2.3 Hz, 1H), 8.11 (d, J=8.0 Hz, 1H), 7.50 (d, J=2.3 Hz, 1H), 7.44 (dd, J=8.0, 1.6 Hz, 1H), 7.33-7.39 (m, 2H), 7.31 (s, 1H), 6.62 (d, J=7.6 Hz, 2H), 5.93 (br s, 1H), 4.62 (t, J=5.6 Hz, 1H), 4.51 (s, 2H), 3.87-3.94 (m, 1H), 3.79 (td, J=8.0, 5.1 Hz, 1H), 3.48-3.60 (m, 3H), 3.28-3.39 (m, 2H), 3.19 (dd, J=9.7, 4.8 Hz, 1H), 2.92-3.04 (m, 3H), 2.13 (dq, J=12.4, 7.7 Hz, 1H), 1.68-1.86 (m, 1H); LCMS: [M +H]⁺=427.39

### Example C-7: 6-(2-amino-5-(4-(piperazin-1-yl)-3-(trifluoromethyl)phenyl)pyridin-3-yl)-3,4-dihydroisoquinolin-1(2H)-one, Comparator 7

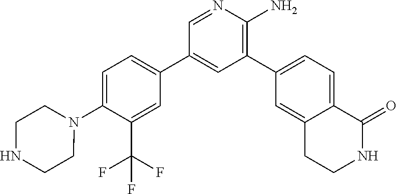 [SMILES: Nc1ncc(-c2ccc(N3CCNCC3)c(C(F)(F)F)c2)cc1-c1ccc2c(c1)CCNC2=O]

Step 1: tert-butyl 4-(4-(6-amino-5-(1-oxo-1,2,3,4-tetrahydroisoquinolin-6-yl)pyridin-3-yl)-2-(trifluoromethyl)phenyl)piperazine-1-carboxylate

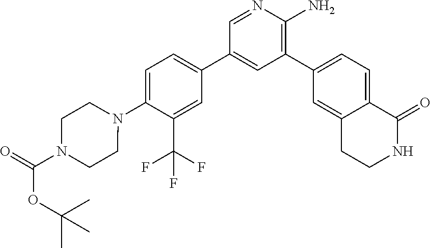 [SMILES: CC(C)(C)OC(=O)N1CCN(c2ccc(-c3cnc(N)c(-c4ccc5c(c4)CCNC5=O)c3)cc2C(F)(F)F)CC1]

To a solution of tert-butyl 4-(4-(4,4,5,5-tetramethyl-1,3,2-dioxaborolan-2-yl)-2-(trifluoromethyl)phenyl)piperazine-1-carboxylate (452.3 mg, 0.991 mmol) and 6-(2-amino-5-bromopyridin-3-yl)-3,4-dihydroisoquinolin-1(2H)-one (303.8 mg, 0.955 mmol) in 1,4-dioxane (6 ml) was added a solution of Cs₂CO₃ (933 mg, 2.86 mmol) in water (1 mL) at RT. The reaction mixture was degassed under argon for 15 min before adding [1,12-bis(diphenylphosphino)ferrocene]dichloropalladium(II) (105 mg, 0.143 mmol). The reaction mixture was heated at 90° C. for overnight until completion of the starting material. Then the reaction mixture was dry loaded onto Celite® and purified by flash silica gel chromatography (eluting with 0-30% CH₂Cl₂ in MeOH) to afford the product (461.7 mg, 85%) as a dark beige powder. LCMS: [M+H]⁺=568.30.

Step 2: 6-(2-amino-5-(4-(piperazin-1-yl)-3-(trifluoromethyl)phenyl)pyridin-3-yl)-3,4-dihydroisoquinolin-1(2H)-one

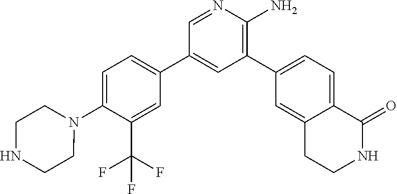 [SMILES: Nc1ncc(-c2ccc(N3CCNCC3)c(C(F)(F)F)c2)cc1-c1ccc2c(c1)CCNC2=O]

To a solution of tert-butyl 4-(4-(6-amino-5-(1-oxo-1,2,3,4-tetrahydroisoquinolin-6-yl)pyridin-3-yl)-2-(trifluoromethyl)phenyl)piperazine-1-carboxylate (461.7 mg, 0.813 mmol) in CH₂Cl₂ (4 mL) was added TFA (2 mL, 26.1 mmol) at RT. The reaction mixture was stirred at RT for 30 min. The solvents were removed and the crude mixture was dissolved in MeOH and passed through a PoraPak Rxn CX (15cc-2g) cartridge in a catch & elute method. The cartridge was washed with MeOH (15 mL), then the solution of product in MeOH was added onto the cartridge. The cartridge was rinsed with MeOH (3×15 mL) and the product was released with a solution of NH3 in MeOH (15 mL® 3N). After removing the solvents under vacuum the product was dry loaded and purified by flash silica gel chromatography (0-30%, MeOH/CH₂Cl₂) to afford the title compound (218.3 mg, 55%) as a beige powder. ¹H NMR (500 MHz, DMSO-d₆) δ 8.33 (d, J=2.4 Hz, 1H), 7.98-7.94 (m, 1H), 7.94-7.89 (m, 2H), 7.84 (d, J=2.1 Hz, 1H), 7.70 (d, J=2.4 Hz, 1H), 7.56-7.49 (m, 2H), 7.48 (s, 1H), 5.95 (s, 2H), 3.41 (dt, J=6.5, 2.6 Hz, 2H), 3.17 (s, 1H), 2.96 (t, J=6.5 Hz, 2H), 2.87-2.79 (m, 8H); LCMS: [M+H]⁺=468.33.

### Example C-8: 6-(2-amino-5-(3,4-dihydro-2H-benzo[b][1,4]dioxepin-7-yl)pyridin-3-yl)-3,4-dihydroisoquinolin-1(2H)-one, Comparator 8

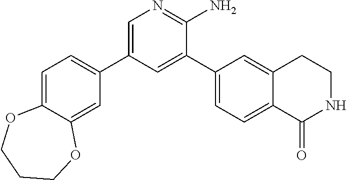 [SMILES: Nc1ncc(-c2ccc3c(c2)OCCCO3)cc1-c1ccc2c(c1)CCNC2=O]

A procedure similar to Example 7 was followed using 3,4-dihydro-2H-1,5-benzodioxepin-7-ylboronic acid (41.8 mg, 0.215 mmol), 6-(2-amino-5-bromopyridin-3-yl)-3,4-dihydroisoquinolin-1(2H)-one (52.7 mg, 0.166 mmol) and DME (3 mL) Cs₂CO₃ (162 mg, 0.497 mmol) in water (1 mL) and [1,1′-bis(diphenylphosphino)ferrocene]dichloropalladium(II) (18.18 mg, 0.025 mmol) afforded after purification by flash silica gel chromatography (eluting with 0-15% CH₂Cl₂ in MeOH) the title compound (47 mg, 73%) as a beige powder. ¹H NMR (500 MHz, DMSO-d₆) δ 8.25 (d, J=2.4 Hz, 1H), 7.94 (br s, 1H), 7.91 (d, J=7.8 Hz, 1H), 7.59 (d, J=2.4 Hz, 1H), 7.51 (dd, J=1.6, 7.9 Hz, 1H), 7.48 (s, 1H), 7.25 (d, J=2.3 Hz, 1H), 7.22 (dd, J=2.3, 8.3 Hz, 1H), 6.99 (d, J=8.3 Hz, 1H), 5.83 (s, 2H), 4.14 (td, J=5.4, 8.7 Hz, 4H), 3.41 (dt, J=2.8, 6.5 Hz, 2H), 2.96 (t, J=6.5 Hz, 2H), 2.11 (quin, J=5.4 Hz, 2H), 0.02-0.01 (m, 1H); LCMS: [M+H]⁺=388.42.

### Example C-9: 6-(2-amino-5-(4-(hexahydropyrrolo[1,2-a]pyrazin-2(1H)-yl)phenyl)pyridin-3-yl)-3,4-dihydroisoquinolin-1(2H)-one, Comparator 9

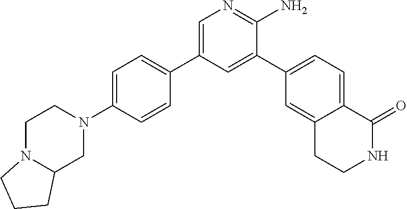 [SMILES: Nc1ncc(-c2ccc(N3CCN4CCCC4C3)cc2)cc1-c1ccc2c(c1)CCNC2=O]

Step 1: 2-(4-iodophenyl)octahydropyrrolo[1,2-a]pyrazine

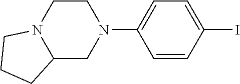 [SMILES: Ic1ccc(N2CCN3CCCC3C2)cc1]

A sealed mixture of octahydropyrrolo[1,2-a]piperazine (509 mg, 4.03 mmol) and K₃PO₄ (2566 mg, 12.09 mmol) in DMF (20 mL) was stirred at RT for 15 min. 1,4-diiodobenzene (1.33 g, 4.03 mmol) was added as a solid. The mixture was then degassed with a stream of N₂ and copper (I) iodide (154 mg, 0.806 mmol) and (R)-(+)-1,1′-bi-2-naphthol (231 mg, 0.806 mmol) were added. The degassing step was repeated. The mixture was then stirred sealed at 40° C. h for 2 d, then diluted with EtOAc, filtered through Celite® and concentrated under reduced pressure. Purification by flash silica gel chromatography (eluting with 8% MeOH in CH₂Cl₂ afforded the product as a light pink solid (825 mg, 62%). LCMS: [M +H]⁺=329.18.

Step 2: 6-(2-amino-5-(4-(hexahydropyrrolo[1,2-a]pyrazin-2(1H)-yl)phenyl)pyridin-3-yl)-3,4-dihydroisoquinolin-1(2H)-one

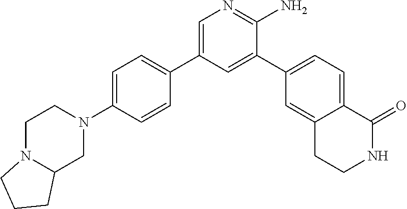 [SMILES: Nc1ncc(-c2ccc(N3CCN4CCCC4C3)cc2)cc1-c1ccc2c(c1)CCNC2=O]

2-(4-Iodophenyl)octahydropyrrolo[1,2-a]pyrazine (62.9 mg, 0.192 mmol), [1,12-bis(diphenylphosphino)ferrocene]dichloropalladium(II) (14.02 mg, 0.019 mmol), 6-(2-amino-5-(4,4,5,5-tetramethyl-1,3,2-dioxaborolan-2-yl)pyridin-3-yl)-3,4-dihydroisoquinolin-1(2H)-one (70 mg, 0.192 mmol) and Cs₂CO₃ (187 mg, 0.575 mmol) were suspended in Water (2 mL). The mixture was degassed (evac-refill with N₂). DME (4 mL) was added, degassing was repeated and the reaction mixture was heated under microwave irradiation at 90° C. for 1 h. The reaction mixture was concentrated under reduced pressure, deposited on Celite® and purified by flash chromatography (eluting with 8% MeOH in CH₂Cl₂ and rinsing with MeOH and eluting with 2 M NH₃ in MeOH afforded the title compound (19.0 mg, 22% yield based on purity of 96%). LCMS: [M+H]⁺=440.56. ¹H NMR (500 MHz, DMSO-d₆) b 8.25 (d, J=2.5 Hz, 1H), 7.88-7.98 (m, 2H), 7.58 (d, J=2.3 Hz, 1H), 7.31-7.52 (m, 4H), 6.99 (d, J=8.8 Hz, 2H), 5.72 (s, 2H), 3.80 (br d, J=9.8 Hz, 1H), 3.65 (br d, J=11.7 Hz, 1H), 3.39-3.44 (m, 2H), 2.93-3.08 (m, 4H), 2.74 (td, J=11.6, 3.2 Hz, 1H), 2.33-2.47 (m, 1H), 2.23 (td, J=11.1, 3.1 Hz, 1H), 1.92-2.10 (m, 2H), 1.64-1.87 (m, 3H), 1.37 (qd, J=11.00, 7.0 Hz, 1H).

### Example C-10: 6-(2-amino-5-(4-(hexahydro-1H-pyrrolo[1,2-a][1,4]diazepin-2(3H)-yl)phenyl)pyridin-3-yl)-3,4-dihydroisoquinolin-1(2H)-one, Comparator 10

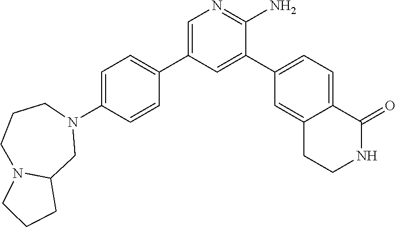 [SMILES: Nc1ncc(-c2ccc(N3CCCN4CCCC4C3)cc2)cc1-c1ccc2c(c1)CCNC2=O]

Step 1: 2-(4-bromophenyl)octahydro-1H-pyrrolo[1,2-a][1,4]diazepine

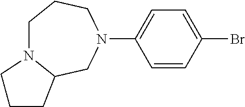 [SMILES: Brc1ccc(N2CCCN3CCCC3C2)cc1]

To a 30 mL vial was added 1-bromo-4-iodobenzene (509 mg, 1.80 mmol), octahydro-1H-pyrrolo[1,2-a][1,4]diazepine (378 mg, 2.70 mmol), tris(dibenzylideneacetone)dipalladium (0) (49.4 mg, 0.054 mmol), 4,5-bis(diphenylphosphino)-9,9-dimethylxanthene (94 mg, 0.162 mmol) and Cs₂CO₃ (1759 mg, 5.40 mmol). The vial was sealed with a cap and septum and then the reaction vessel was evacuated and backfilled with nitrogen. Toluene (4 mL) was added and the reaction vessel was evacuated and backfilled with nitrogen an additional time. The reaction was then heated at 100° C. for 16 h, concentrated onto Celite® and purified by flash silica gel chromatography (eluting with 100% CH₂Cl₂ to 95-5-1 CH₂Cl₂/MeOH/NH₄OH) to give the product (161 mg, 30%) as a brown oil. LCMS: [M+H]⁺=297.07.

Step 2: 6-(2-amino-5-(4-(hexahydro-1H-pyrrolo[1,2-a][1,4]diazepin-2(3H)-yl)phenyl)pyridin-3-yl)-3,4-dihydroisoquinolin-1(2H)-one

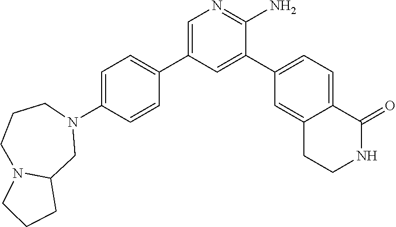 [SMILES: Nc1ncc(-c2ccc(N3CCCN4CCCC4C3)cc2)cc1-c1ccc2c(c1)CCNC2=O]

In a microwave vial with magnetic stir bar was placed 6-(2-amino-5-(4,4,5,5-tetramethyl-1,3,2-dioxaborolan-2-yl)pyridin-3-yl)-3,4-dihydroisoquinolin-1(2H)-one (119 mg, 0.325 mmol), 2-(4-bromophenyl)octahydro-1H-pyrrolo[1,2-a][1,4]diazepine (80 mg, 0.271 mmol), [1,12-bis(diphenylphosphino)ferrocene]dichloropalladium(II) (19.83 mg, 0.027 mmol), K₃PO₄ (0.417 mL, 0.542 mmol), DME (4 mL) and water (2 mL). The flask was sealed, then heated to 90° C. for 1 h in a microwave apparatus, loaded onto Celite® and purified by flash silica gel chromatography (eluting with 100% CH₂Cl₂ to 95-5-1 CH₂Cl₂/MeOH/NH₄OH), followed by purification by reverse phase chromatography (C₁₈ column, MeCN—H₂O) to give the product (49 mg, 38%) as a tan solid. ¹H NMR (500 MHz, DMSO-d₆) δ 8.21 (d, J=2.3 Hz, 1H), 7.97-7.89 (m, 2H), 7.54 (d, J=2.4 Hz, 1H), 7.49 (dd, J=7.9, 1.5 Hz, 1H), 7.46 (s, 1H), 7.42 (d, J=8.8 Hz, 2H), 6.74 (d, J=8.9 Hz, 2H), 5.64 (s, 2H), 3.81 (dd, J=13.9, 2.1 Hz, 1H), 3.66 (dt, J=14.5, 5.5 Hz, 1H), 3.51-3.44 (m, 2H), 2.98-2.91 (m, 4H), 2.48-2.45 (m, 1H), 2.24 (q, J=8.9 Hz, 1H), 2.04 (td, J=12.0, 4.2 Hz, 1H), 1.98-1.88 (m, 2H), 1.81-1.72 (m, 1H), 1.72-1.58 (m, 2H), 1.45-1.35 (m, 1H); LCMS: [M+H]⁺=454.42.

### Example C-11: 6-(2-amino-5-(4-(piperidin-4-yl)phenyl)pyridin-3-yl)-7-fluoro-3,4-dihydroisoquinolin-1(2H)-one, Comparator 11

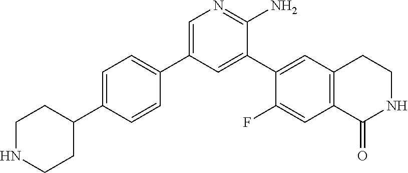 [SMILES: Nc1ncc(-c2ccc(C3CCNCC3)cc2)cc1-c1cc2c(cc1F)C(=O)NCC2]

A mixture of 6-(2-amino-5-(4,4,5,5-tetramethyl-1,3,2-dioxaborolan-2-yl)pyridin-3-yl)-7-fluoro-3,4-dihydroisoquinolin-1(2H)-one (383 mg, 0.999 mmol), Cs₂CO₃ (814 mg, 2.499 mmol), 4-(4-bromophenyl)piperidine (200 mg, 0.833 mmol) and [1,1′-bis(diphenylphosphino)ferrocene]dichloropalladium(II) (73.1 mg, 0.100 mmol) in DME (10 mL) and water (2.0 mL) in a microwave vial was flushed with argon and heated in a microwave reactor at 85° C. for 2 h. The reaction mixture was then partitioned between brine and EtOAc, the organic phase was separated, the aqueous phase was extracted with CHCl₃/IPA(4:1) (x 3), the combined organic layers were dried over Na₂SO₄ and concentrated onto Celite®. Purification using silica gel chromatography eluting with CH₂Cl₂ containing 0-7% MeOH and 0-0.7% NH₄OH afforded the title compound as a beige solid (216 mg, 59%). ¹H NMR: (500 MHz, DMSO-d₆) δ 8.37-8.28 (m, 1H), 8.14-8.06 (m, 1H), 7.67-7.59 (m, 2H), 7.58-7.52 (m, 2H), 7.47-7.40 (m, 1H), 7.29-7.21 (m, 2H), 5.89-5.77 (m, 2H), 4.20-3.96 (m, 1H), 3.46-3.40 (m, 2H), 3.08-3.00 (m, 2H), 2.98-2.91 (m, 2H), 2.65-2.54 (m, 3H), 1.74-1.65 (m, 2H), 1.60-1.44 (m, 2H); LCMS: [M+H]⁺=417.55

### C: Biological Assays

### (a) HPK1 Human STE Kinase Enzymatic Radiometric Assay

A stock solution of 10 mM of test compound is prepared in DMSO. The compound plate was prepared by 3-fold and 9-point serial dilutions. Recombinant (1-346) HPK1 (h) is incubated with 8 mM MOPS pH 7.0, 0.2 mM EDTA, 0.33 mg/mL myelin basic protein, 10 mM MgAcetate and [gamma-33P]-ATP (specific activity and concentration as required). The reaction is initiated by The addition of the Mg/ATP mix. After incubation for 40 minutes at RT, the reaction is stopped by the addition of phosphoric acid to a concentration of 0.5%. 10 uL of the reaction is then spotted onto a P30 filtermat and washed four times for 4 minutes in 0.425% phosphoric acid and once in methanol prior to drying and scintillation counting. For more details of kinase assay protocols, see: Gao, Y. et. al.; Biochem J.451 (2): 313-328, 2013. Compounds of the application showed activity as inhibitors of HPK1 having IC₅₀'s in the following ranges: A: 0.1-10 nM; B: 11-100 nM; C: 101-1000 nM; D: >1000 nM. Specific ranges for exemplary compounds of Formula (I) are shown in Table 2.

### (b) Lck Human STE Kinase Enzymatic Radiometric Assay

Lck (h) is incubated with 50 mM Tris pH 7.5, 0.1 mM EGTA, 0.1 mM Na3VO4, 250 uM KVEKIGEGTYGVVYK (Cdc2 peptide), 10 mM MgAcetate and [gamma-33P]-ATP (specific activity and concentration as required). The reaction is initiated by the addition of the Mg/ATP mix. After incubation for 40 minutes at RT, the reaction is stopped by the addition of phosphoric acid to a concentration of 0.5%. 10 uL of the reaction is then spotted onto a P30 filtermat and washed four times for 4 minutes in 0.425% phosphoric acid and once in methanol prior to drying and scintillation counting. For more details on the kinase assay procedures see: Gao, Y. et. al.; Biochem J., 451 (2): 313-328, 2013. Generally, and advantageously, compounds of the application showed significantly less inhibition of Lck compared with inhibition of HPK1 (see Table 2 where IC₀o's are reported the following ranges: A: 0.1-10 nM; B: 11-100 nM; C: 101-1000 nM; D: >1000 nM for the compounds of Formula (I)).

In some embodiments the presence of a halo substituent on the amino pyridine ring provides compounds with improved selectivity for inhibition of HPK1 vs Lck (see, for example, Table 3 and Table 4). (c) Human Jurkat T Lymphocyte Anti-proliferation Assay

Assay principle: Jurkat cells are incubated with various concentrations of test compounds for 72 h, and cell proliferation/cytotoxicity is measured via detection of ATP production.

Literature: See Cree, I A et. al.; Toxicol In Vitro., 11 (5): 553-556, 1997 for additional information of ATP detection.

Assay Procedure: Jurkat cells (cultured in a-MEM media with 10% FBS) are seeded at 2000 cells/well (50 μL) in a 384-well well black culture plate (Perkin Elmer). Test compounds (in DMSO) are added to cells using the HP digital dispenser, and incubated at 37° C., 5% CO₂ for 72 hours. ATP production is measured by adding 40 μL/well of ATPLite-1-Step reagent (Perkin Elmer), incubating for 5 min at RT with shaking followed by detection of luminescent signal using an Envision plate reader (Perkin Elmer). Data is normalized to untreated cells, and plotted using XLFit. IC₅₀ values are calculated using a 4 parameter dose-response equation by fitting the curve of % inhibition versus Log of compound concentration.

Results: In an embodiment, exemplary compounds of the application had IC₅₀'s in the range of 0.1 to >10 μM in this assay. In an embodiment, exemplary compounds of the application had IC₅₀'s in the range of 1.0 to >10 uM in this assay.

### (d) Human Jurkat T Lymphocyte IL-2 Release Assay

Assay principle: Stimulation of TCR signaling via anti-CD3/CD28 antibody treatment in Jurkat T cells will lead to IL-2 secretion into the culture media, detected by a luminescent energy transfer bead immunoassay (IL-2 AlphaLISA kit).

Literature: See Cauchon, E. et. al.; Analytical Biochemistry., 388 (1): 134-139, 2009 for additional information on AlphaLISA detection.

Assay procedure: Jurkat cells (cultured in a-MEM media with 10% FBS) are seeded at 0.2×10⁶ cells/well (100 μL) in a 96-well round bottom culture plate (Greiner). Test compounds (in DMSO) are added to cells using an HP digital dispenser, and incubated for 15 mins at RT. Cells are stimulated with 15 μL/mL (v/v) of soluble CD3/CD28 antibodies in a-MEM media (Stem Cell Technologies; 50 μL/well, 150 μL final assay volume), and incubated at 37° C., 5% CO₂ for 4 hours. Cells are centrifuged at 1500 rpm for 5 mins at RT, and 5 μL of culture media is transferred to a 96 well % area white plate (Perkin Elmer). IL-2 is detected by adding 20 μL/well of a mixture of both Acceptor beads and Biotin anti-IL-2 antibody (1:200 dilution of each), and incubated for 1 hour at RT with shaking. 25 μL/well of Donor beads (1:63 dilution) are then added and incubated in the dark for 30 mins at RT with shaking, followed by detection of luminescent signal using an Envision plate reader (Perkin Elmer). Data is normalized to untreated/stimulated cells, and plotted using XLFit. EC₅₀ values are calculated using a 4 parameter dose-response equation by fitting the curve of % stimulation versus Log of compound concentration.

Results: In an embodiment, exemplary compounds of the application had EC₁₅₀'s in the range of 0.01-5 uM in this assay. In an embodiment, exemplary compounds of the application had EC₁₅₀'s in the range of 0.01-1.3 μM in this assay.

(e) p-SLP76 S376 Phosphorylation Inhibition Assay

Assay principle: Stimulation of TCR signaling via anti-CD3/CD28 antibody treatment in Jurkat T cells will lead to phosphorylation of HPK1 at Serine 376, detected by a luminescent energy transfer bead immunoassay (p-SPL76 AlphaLISA kit).

Literature: See Cauchon, E. et. al.; Analytical Biochemistry., 388 (1): 134-139, 2009 for additional information on AlphaLISA detection.

Assay procedure: Jurkat cells (cultured in a-MEM media with 10% FBS) are collected and centrifuged, and seeded in a 96-well % area white plate (Perkin Elmer) at 0.1×10⁶ cells/well in HBSS (25 μL). Test compounds (in DMSO) are added to cells using an HP digital dispenser, and incubated for 15 mins at RT. Cells are stimulated with 15 μl/mL (v/v) of soluble CD3/CD28 antibodies in HBSS (Stem Cell Technologies; 25 μL, 50 μL final assay volume), and incubated at 37° C., 5% CO₂ for 1 hour. Cells are lysed with 10 μL/well of lysis buffer followed by incubation at RT for 10 mins with shaking. p-SLP76 S376 is detected by adding 15 μl/well of Acceptor beads (1:50 dilution), and incubated for 1 hour at RT with shaking. 15 μL/well of Donor beads (1:50 dilution) are then added and incubated at RT in the dark for 1 hour with shaking, followed by detection of luminescent signal using an Envision plate reader (Perkin Elmer). Data is normalized to untreated/stimulated cells, and plotted using Graph Pad Prism. IC₅₀ values are calculated using a 4 parameter dose-response equation by fitting the curve of % inhibition versus Log of compound concentration. (e) Glucose kinase (GIk) Assay

GLK(h) (MAP4K3(h)) is incubated with 8 mM MOPS pH 7.0, 0.2 mM EDTA, 250 μM RLGRDKYKTLRQIRQ, 10 mM Magnesium acetate and [9-33P-ATP] (specific activity and concentration as required). The reaction is initiated by the addition of the Mg/ATP mix. After incubation for 40 minutes at room temperature, the reaction is stopped by the addition of phosphoric acid to a concentration of 0.5%. 10 μl of the reaction is then spotted onto a P30 filtermat and washed four times for 4 minutes in 0.425% phosphoric acid and once in methanol prior to drying and scintillation counting. For more details on the kinase assay procedures see: Gao, Y. et. al.; Biochem J., 451 (2): 313-328, 2013.

Results: In an embodiment, exemplary compounds of the application had IC₅₀'s in the range of greater than 10× greater than the value for HPK1 in this assay. In an embodiment, exemplary compounds of the application had IC₅₀'s in the range of at least 20× greater than HPK1. in this assay.

While the present application has been described with reference to what are presently considered to be the preferred examples, it is to be understood that the application is not limited to the disclosed examples. To the contrary, the present application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 2

| | | | HPK1 | Lck |
|---|---|---|---|---|
| Com- | | | IC₅₀ | IC₅₀ |
| pound | | | nM | nM |
| I.D | IUPAC Chemical Name | Structures | Range* | Range* |
| I-1 | 6-(2-amino-4-fluoro-5-(4- (4-methylpiperazin-1- yl)phenyl)pyridin-3-yl)- 3,4-dihydroisoquinolin- 1(2H)-one | 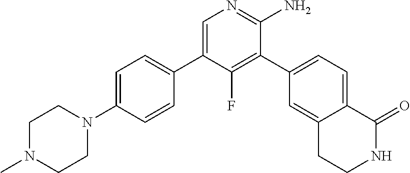 [SMILES: CN1CCN(c2ccc(-c3cnc(N)c(-c4ccc5c(c4)CCNC5=O)c3F)cc2)CC1] | B | C |
| I-2 | 6-(2-amino-4-fluoro-5-(4- morpholinophenyl)pyridin- 3-yl)-3,4- dihydroisoquinolin-1(2H)-one | 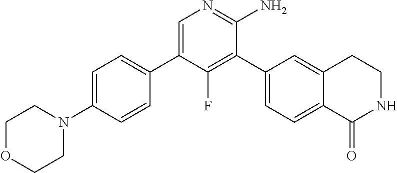 [SMILES: Nc1ncc(-c2ccc(N3CCOCC3)cc2)c(F)c1-c1ccc2c(c1)CCNC2=O] | B | D |
| I-3 | 6-(2-amino-4-fluoro-5-(4- (isopropylsulfonyl)phenyl) pyridin-3-yl)-3,4- dihydroisoquinolin- 1(2H)-one | 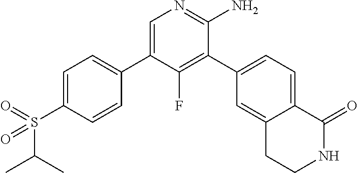 [SMILES: CC(C)S(=O)(=O)c1ccc(-c2cnc(N)c(-c3ccc4c(c3)CCNC4=O)c2F)cc1] | B | D |
| I-4 | (R)-6-(2-amino-4-fluoro- 5-(4-(2- isopropylmorpholino)phe- nyl)pyridin-3-yl)-3,4- dihydroisoquinolin- 1(2H)-one | 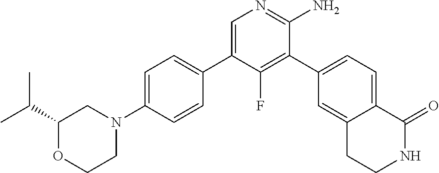 [SMILES: CC(C)[C@@H]1CN(c2ccc(-c3cnc(N)c(-c4ccc5c(c4)CCNC5=O)c3F)cc2)CCO1] | B | D |
| I-5 | (S)-6-(2-amino-4-fluoro- 5-(4-(2- isopropylmorpholino)phe- nyl)pyridin-3-yl)-3,4- dihydroisoquinolin- 1(2H)-one | 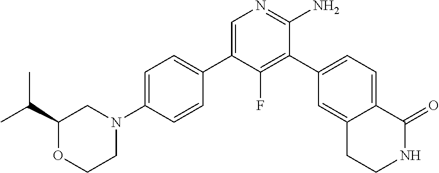 [SMILES: CC(C)[C@H]1CN(c2ccc(-c3cnc(N)c(-c4ccc5c(c4)CCNC5=O)c3F)cc2)CCO1] | B | D |
| I-6 | (R)-6-(2-amino-4-fluoro- 5-(2-fluoro-4-(2- isopropylmorpholino)phe- nyl)pyridin-3-yl)-3,4- dihydroisoquinolin- 1(2H)-one | 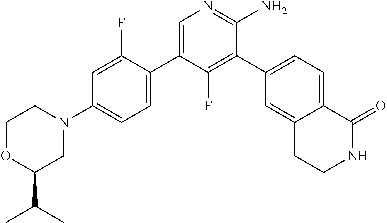 [SMILES: CC(C)[C@@H]1CN(c2ccc(-c3cnc(N)c(-c4ccc5c(c4)CCNC5=O)c3F)c(F)c2)CCO1] | B | D |
| I-7 | (S)-6-(2-amino-4-fluoro- 5-(2-fluoro-4-(2- isopropylmorpholino)phe- nyl)pyridin-3-yl)-3,4- dihydroisoquinolin- 1(2H)-one | 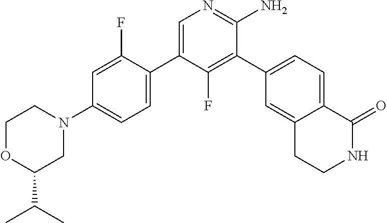 [SMILES: CC(C)[C@H]1CN(c2ccc(-c3cnc(N)c(-c4ccc5c(c4)CCNC5=O)c3F)c(F)c2)CCO1] | C | D |
| I-8 | 6-(2-amino-6-fluoro-5-(4- (4-methylpiperazin-1- yl)phenyl)pyridin-3-yl)- 3,4-dihydroisoquinolin- 1(2H)-one | 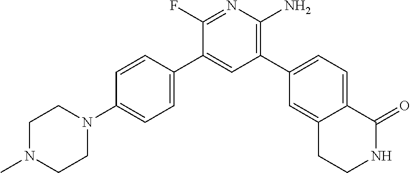 [SMILES: CN1CCN(c2ccc(-c3cc(-c4ccc5c(c4)CCNC5=O)c(N)nc3F)cc2)CC1] | A | D |
| I-9 | (R)-6-(2-amino-6-fluoro- 5-(4-(2- isopropylmorpholino)phe- nyl)pyridin-3-yl)-3,4- dihydroisoquinolin- 1(2H)-one | 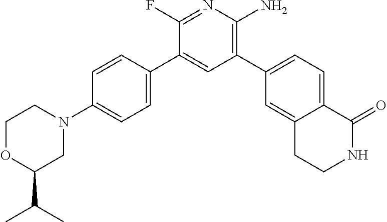 [SMILES: CC(C)[C@@H]1CN(c2ccc(-c3cc(-c4ccc5c(c4)CCNC5=O)c(N)nc3F)cc2)CCO1] | A | D |
| I-10 | (S)-6-(2-amino-6-fluoro- 5-(4-(2- isopropylmorpholino)phe- nyl)pyridin-3-yl)-3,4- dihydroisoquinolin- 1(2H)-one | 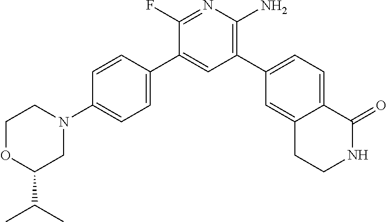 [SMILES: CC(C)[C@H]1CN(c2ccc(-c3cc(-c4ccc5c(c4)CCNC5=O)c(N)nc3F)cc2)CCO1] | B | D |
| I-11 | (R)-6-(2-amino-6-fluoro- 5-(2-fluoro-4-(2- isopropylmorpholino)phe- nyl)pyridin-3-yl)-3,4- dihydroisoquinolin- 1(2H)-one | 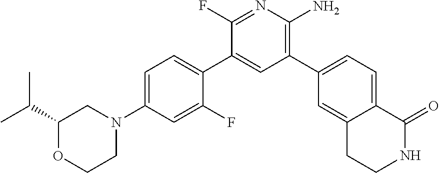 [SMILES: CC(C)[C@@H]1CN(c2ccc(-c3cc(-c4ccc5c(c4)CCNC5=O)c(N)nc3F)c(F)c2)CCO1] | A | D |
| I-12 | (S)-6-(2-amino-6-fluoro- 5-(2-fluoro-4-(2- isopropylmorpholino)phe- nyl)pyridin-3-yl)-3,4- dihydroisoquinolin- 1(2H)-one | 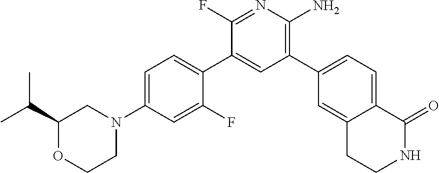 [SMILES: CC(C)[C@H]1CN(c2ccc(-c3cc(-c4ccc5c(c4)CCNC5=O)c(N)nc3F)c(F)c2)CCO1] | B | D |
| I-13 | 6-(2-amino-6-fluoro-5-(4- ((1R,5S)-8-methyl-3,8- diazabicyclo[3.2.1]octan- 3-yl)phenyl)pyridin-3-yl)- 3,4-dihydroisoquinolin- 1(2H)-one | 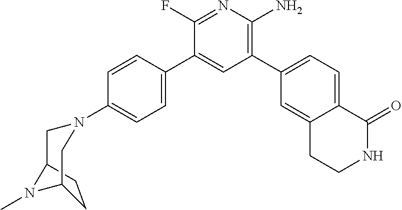 [SMILES: CN1C2CCC1CN(c1ccc(-c3cc(-c4ccc5c(c4)CCNC5=O)c(N)nc3F)cc1)C2] | A | C |
| I-14 | 6-(2-amino-6-fluoro-5-(4- ((1R,5S)-3-methyl-3- azabicyclo[3.1.0]hexan- 1-yl)phenyl)pyridin-3-yl)- 3,4-dihydroisoquinolin- 1(2H)-one | 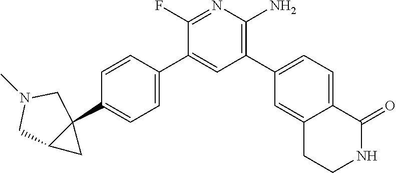 [SMILES: CN1C[C@H]2C[C@@]2(c2ccc(-c3cc(-c4ccc5c(c4)CCNC5=O)c(N)nc3F)cc2)C1] | A | D |
| I-15 | 6-(2-amino-6-fluoro-5-(4- (isopropylsulfonyl)phenyl) pyridin-3-yl)-3,4- dihydroisoquinolin- 1(2H)-one | 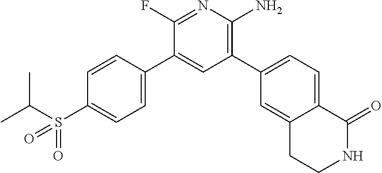 [SMILES: CC(C)S(=O)(=O)c1ccc(-c2cc(-c3ccc4c(c3)CCNC4=O)c(N)nc2F)cc1] | A | D |
| I-16 | 6-(2-amino-6-fluoro-5-(4- morpholinophenyl)pyridin- 3-yl)-3,4- dihydroisoquinolin- 1(2H)-one | 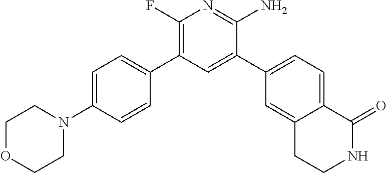 [SMILES: Nc1nc(F)c(-c2ccc(N3CCOCC3)cc2)cc1-c1ccc2c(c1)CCNC2=O] | A | D |
| I-17 | 6-(2-amino-6-fluoro-5-(4- (piperazin-1- yl)phenyl)pyridin-3-yl)- 3,4-dihydroisoquinolin- 1(2H)-one | 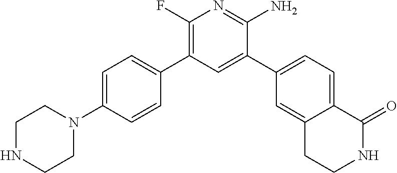 [SMILES: Nc1nc(F)c(-c2ccc(N3CCNCC3)cc2)cc1-c1ccc2c(c1)CCNC2=O] | A | D |
| I-18 | 6-(2-amino-6-fluoro-5-(4- (5-methyl-2,5- diazabicyclo[2.2.1]heptan- 2-yl)phenyl)pyridin-3- yl)-3,4- dihydroisoquinolin- 1(2H)-one | 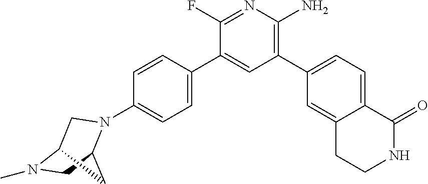 [SMILES: CN1C[C@@H]2C[C@H]1CN2c1ccc(-c2cc(-c3ccc4c(c3)CCNC4=O)c(N)nc2F)cc1] | A | D |
| I-19 | 6-(2-amino-6-fluoro-5-(4- morpholinophenyl)pyridin- 3-yl)-7-fluoro-3,4- dihydroisoquinolin- 1(2H)-one | 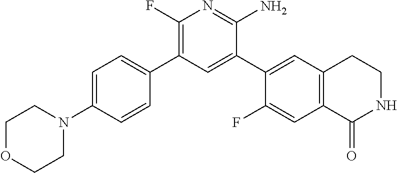 [SMILES: Nc1nc(F)c(-c2ccc(N3CCOCC3)cc2)cc1-c1cc2c(cc1F)C(=O)NCC2] | A | D |
| I-20 | 6-(2-amino-6-fluoro-5-(4- (4-(2- fluoroethyl)piperazin-1- yl)phenyl)pyridin-3-yl)- 3,4-dihydroisoquinolin- 1(2H)-one | 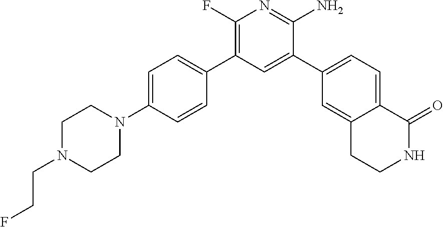 [SMILES: Nc1nc(F)c(-c2ccc(N3CCN(CCF)CC3)cc2)cc1-c1ccc2c(c1)CCNC2=O] | A | D |
| I-21 | 6-(2-amino-5-(4-(4-(2,2- difluoroethyl)piperazin-1- yl)phenyl)-6- fluoropyridin-3-yl)-3,4- dihydroisoquinolin- 1(2H)-one | 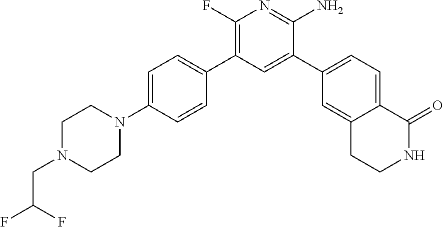 [SMILES: Nc1nc(F)c(-c2ccc(N3CCN(CC(F)F)CC3)cc2)cc1-c1ccc2c(c1)CCNC2=O] | A | D |
| I-22 | 6-(2-amino-6-fluoro-5-(4- (piperidin-4- yloxy)phenyl)pyridin-3- yl)-3,4- dihydroisoquinolin- 1(2H)-one | 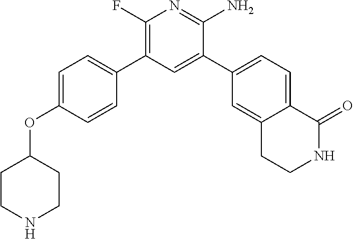 [SMILES: Nc1nc(F)c(-c2ccc(OC3CCNCC3)cc2)cc1-c1ccc2c(c1)CCNC2=O] | A | D |
| I-23 | 6-(2-amino-6-fluoro-5-(4- (hexahydropyrazino[2,1- c][1,4]oxazin-8(1H)- yl)phenyl)pyridin-3-yl)- 3,4-dihydroisoquinolin- 1(2H)-one | 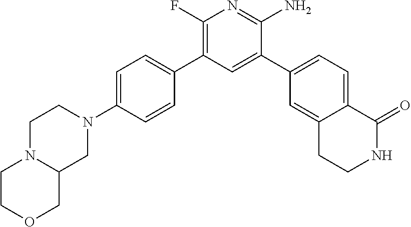 [SMILES: Nc1nc(F)c(-c2ccc(N3CCN4CCOCC4C3)cc2)cc1-c1ccc2c(c1)CCNC2=O] | A | D |
| I-24 | (S)-6-(2-amino-6-fluoro- 5-(4-(4-(2- methoxypropyl)piperazin- 1-yl)phenyl)pyridin-3-yl)- 3,4-dihydroisoquinolin- 1(2H)-one | 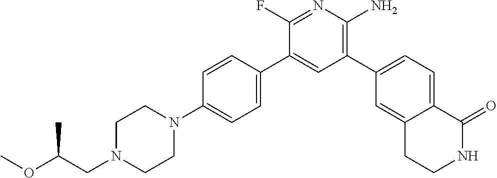 [SMILES: CO[C@@H](C)CN1CCN(c2ccc(-c3cc(-c4ccc5c(c4)CCNC5=O)c(N)nc3F)cc2)CC1] | A | D |
| I-25 | (R)-6-(2-amino-6-fluoro- 5-(4-(4-(2- methoxypropyl)piperazin- 1-yl)phenyl)pyridin-3-yl)- 3,4-dihydroisoquinolin- 1(2H)-one | 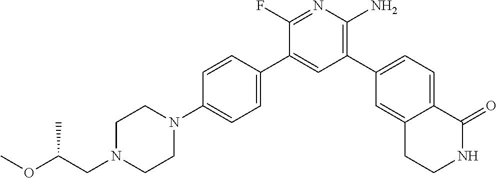 [SMILES: CO[C@H](C)CN1CCN(c2ccc(-c3cc(-c4ccc5c(c4)CCNC5=O)c(N)nc3F)cc2)CC1] | A | D |
| I-26 | 6-(6′-amino-2′-fluoro-5- morpholino-[2,3′- bipyridin]-5′-yl)-3,4- dihydroisoquinolin- 1(2H)-one | 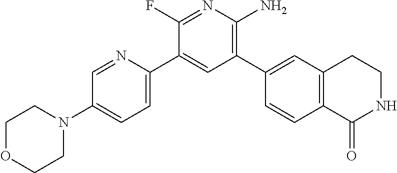 [SMILES: Nc1nc(F)c(-c2ccc(N3CCOCC3)cn2)cc1-c1ccc2c(c1)CCNC2=O] | A | D |
| I-27 | 6-(6′-amino-2′-fluoro-5- (4-methylpiperazin-1-yl)- [2,3′-bipyridin]-5′-yl)-3,4- dihydroisoquinolin- 1(2H)-one acetate | 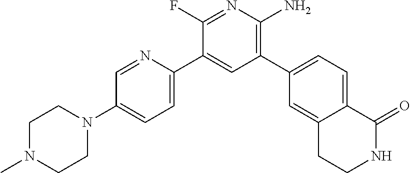 [SMILES: CN1CCN(c2ccc(-c3cc(-c4ccc5c(c4)CCNC5=O)c(N)nc3F)nc2)CC1] | A | D |
| I-28 | 6-(2-amino-6-fluoro-5-(4- (hydroxydimethylsilyl)phe- nyl)pyridin-3-yl)-3,4- dihydroisoquinolin- 1(2H)-one | 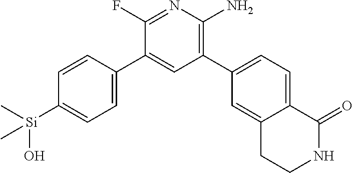 [SMILES: C[Si](C)(O)c1ccc(-c2cc(-c3ccc4c(c3)CCNC4=O)c(N)nc2F)cc1] | B | D |
| I-29 | 6-(2-amino-6-fluoro-5-(4- (4-(2- methoxyethyl)piperazin- 1-yl)phenyl)pyridin-3-yl)- 3,4-dihydroisoquinolin- 1(2H)-one | 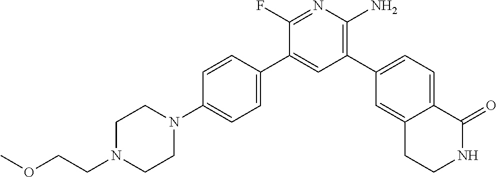 [SMILES: COCCN1CCN(c2ccc(-c3cc(-c4ccc5c(c4)CCNC5=O)c(N)nc3F)cc2)CC1] | A | D |
| I-30 | 6-(2-amino-6-fluoro-5-(4- (piperidin-4- yl)phenyl)pyridin-3-yl)- 3,4-dihydroisoquinolin- 1(2H)-one | 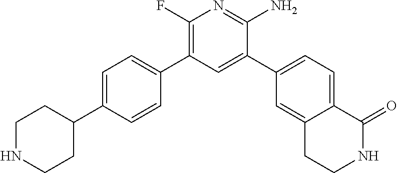 [SMILES: Nc1nc(F)c(-c2ccc(C3CCNCC3)cc2)cc1-c1ccc2c(c1)CCNC2=O] | A | D |
| I-31 | 6-(2-amino-6-fluoro-5-(4- (1-methyl-5,6-dihydro- 1,2,4-triazin-4(1H)- yl)phenyl)pyridin-3-yl)- 3,4-dihydroisoquinolin- 1(2H)-one | 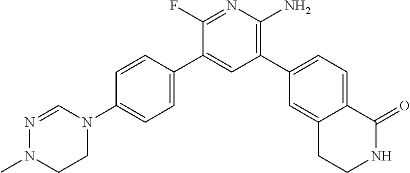 [SMILES: CN1CCN(c2ccc(-c3cc(-c4ccc5c(c4)CCNC5=O)c(N)nc3F)cc2)C=N1] | A | D |
| I-32 | 5-(2-amino-6-fluoro-5-(4- (4-methylpiperazin-1- yl)phenyl)pyridin-3- yl)isoindolin-1-one | 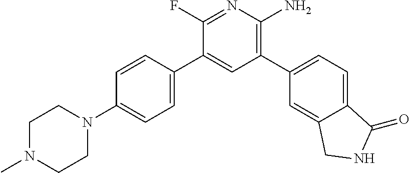 [SMILES: CN1CCN(c2ccc(-c3cc(-c4ccc5c(c4)CNC5=O)c(N)nc3F)cc2)CC1] | B | D |
| I-33 | 5-(2-amino-6-fluoro-5-(4- (4-methylpiperazin-1- yl)phenyl)pyridin-3-yl)- 3,3-dimethylisoindolin-1- one | 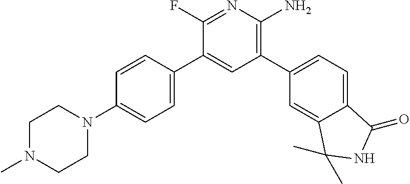 [SMILES: CN1CCN(c2ccc(-c3cc(-c4ccc5c(c4)C(C)(C)NC5=O)c(N)nc3F)cc2)CC1] | A | D |
| I-34 | 6-(5-(4-((1- acetylpiperidin-4- yl)oxy)phenyl)-2-amino- 6-fluoropyridin-3-yl)-3,4- dihydroisoquinolin- 1(2H)-one | 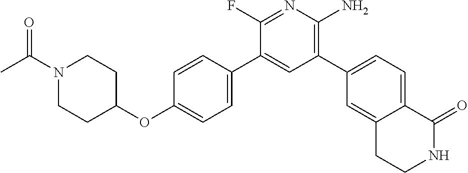 [SMILES: CC(=O)N1CCC(Oc2ccc(-c3cc(-c4ccc5c(c4)CCNC5=O)c(N)nc3F)cc2)CC1] | A | D |
| I-35 | 6-(2-amino-6-fluoro-5-(4- ((tetrahydro-2H-pyran-4- yl)oxy)phenyl)pyridin-3- yl)-3,4- dihydroisoquinolin- 1(2H)-one | 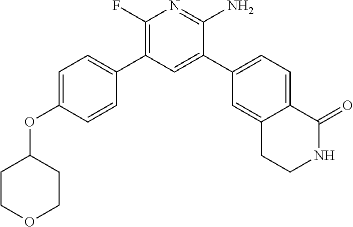 [SMILES: Nc1nc(F)c(-c2ccc(OC3CCOCC3)cc2)cc1-c1ccc2c(c1)CCNC2=O] | A | D |
| I-36 | 5-(2-amino-6-fluoro-5-(4- ((1R,5S)-3-methyl-3- azabicyclo[3.1.0]hexan- 1-yl)phenyl)pyridin-3-yl)- 3,3-dimethylisoindolin-1- one | 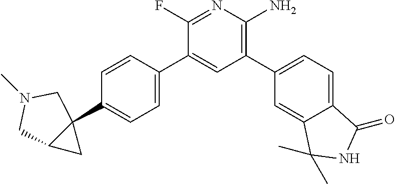 [SMILES: CN1C[C@H]2C[C@@]2(c2ccc(-c3cc(-c4ccc5c(c4)C(C)(C)NC5=O)c(N)nc3F)cc2)C1] | A | D |
| I-37 | 5-(2-amino-6-fluoro-5-(4- (1-methyl-5,6-dihydro- 1,2,4-triazin-4(1H)- yl)phenyl)pyridin-3-yl)- 3,3-dimethylisoindolin-1- one 2,2,2-trifluoroacetate | 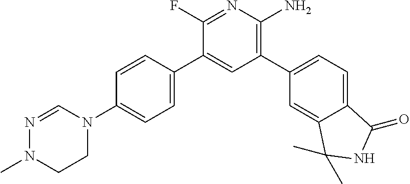 [SMILES: CN1CCN(c2ccc(-c3cc(-c4ccc5c(c4)C(C)(C)NC5=O)c(N)nc3F)cc2)C=N1] | B | D |
| I-38 | (R)-6-(2-amino-6-fluoro- 5-(4- (hexahydropyrazino[2,1- c][1,4]oxazin-8(1H)- yl)phenyl)pyridin-3-yl)- 3,4-dihydroisoquinolin- 1(2H)-one | 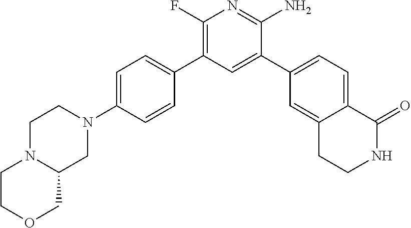 [SMILES: Nc1nc(F)c(-c2ccc(N3CCN4CCOC[C@H]4C3)cc2)cc1-c1ccc2c(c1)CCNC2=O] | A | D |
| I-39 | (S)-6-(2-amino-6-fluoro- 5-(4- (hexahydropyrazino[2,1- c][1,4]oxazin-8(1H)- yl)phenyl)pyridin-3-yl)- 3,4-dihydroisoquinolin- 1(2H)-one | 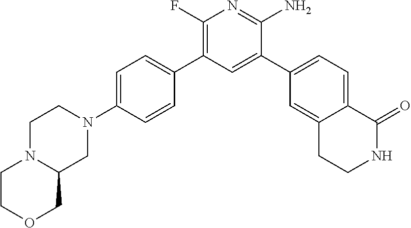 [SMILES: Nc1nc(F)c(-c2ccc(N3CCN4CCOC[C@@H]4C3)cc2)cc1-c1ccc2c(c1)CCNC2=O] | A | D |
| I-40 | 6-(2-amino-6-fluoro-5-(3- fluoro-4-(4- methylpiperazin-1- yl)phenyl)pyridin-3-yl)- 3,4-dihydroisoquinolin- 1(2H)-one | 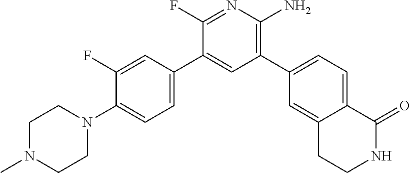 [SMILES: CN1CCN(c2ccc(-c3cc(-c4ccc5c(c4)CCNC5=O)c(N)nc3F)cc2F)CC1] | A | D |
| I-41 | 6-(2-amino-5-(4-(4- ethylpiperazin-1- yl)phenyl)-6- fluoropyridin-3-yl)-3,4- dihydroisoquinolin- 1(2H)-one | 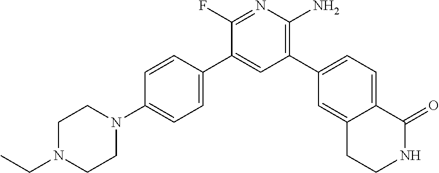 [SMILES: CCN1CCN(c2ccc(-c3cc(-c4ccc5c(c4)CCNC5=O)c(N)nc3F)cc2)CC1] | A | D |
| I-42 | 6-(2-amino-6-fluoro-5-(4- (4-isopropylpiperazin-1- yl)phenyl)pyridin-3-yl)- 3,4-dihydroisoquinolin- 1(2H)-one | 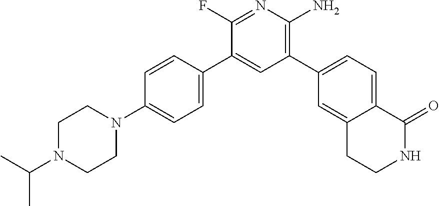 [SMILES: CC(C)N1CCN(c2ccc(-c3cc(-c4ccc5c(c4)CCNC5=O)c(N)nc3F)cc2)CC1] | A | D |
| I-43 | 6-(2-amino-5-(4-(1-(2,2- difluoroethyl)piperidin-4- yl)phenyl)-6- fluoropyridin-3-yl)-3,4- dihydroisoquinolin- 1(2H)-one | 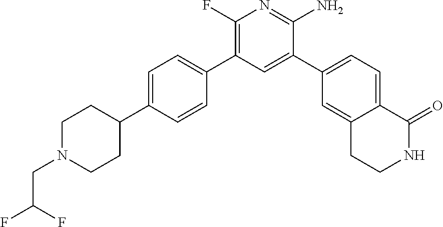 [SMILES: Nc1nc(F)c(-c2ccc(C3CCN(CC(F)F)CC3)cc2)cc1-c1ccc2c(c1)CCNC2=O] | A | D |
| I-44 | 6-(2-amino-6-fluoro-5-(4- ((1-methylpiperidin-4- yl)oxy)phenyl)pyridin-3- yl)-3,4- dihydroisoquinolin- 1(2H)-one | 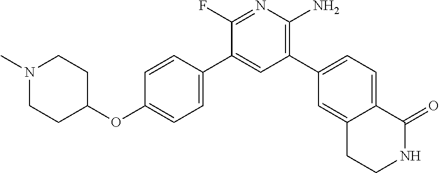 [SMILES: CN1CCC(Oc2ccc(-c3cc(-c4ccc5c(c4)CCNC5=O)c(N)nc3F)cc2)CC1] | A | D |
| I-45 | 6-(2-amino-5-(4-((2- (dimethylamino)ethyl)(meth- yl)amino)phenyl)-6- fluoropyridin-3-yl)-3,4- dihydroisoquinolin- 1(2H)-one | 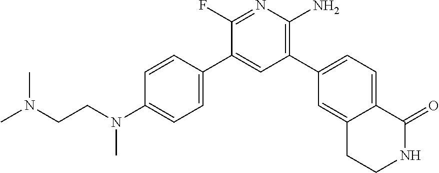 [SMILES: CN(C)CCN(C)c1ccc(-c2cc(-c3ccc4c(c3)CCNC4=O)c(N)nc2F)cc1] | A | D |
| I-46 | (R)-6-(2-amino-6-fluoro- 5-(4-(2-methylpyrrolidin- 1-yl)phenyl)pyridin-3-yl)- 3,4-dihydroisoquinolin- 1(2H)-one | 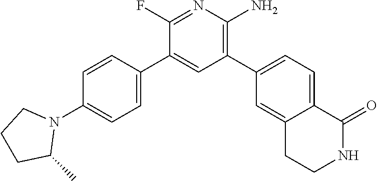 [SMILES: C[C@@H]1CCCN1c1ccc(-c2cc(-c3ccc4c(c3)CCNC4=O)c(N)nc2F)cc1] | B | D |
| I-47 | (R)-6-(2-amino-6-fluoro- 5-(2-fluoro-4-(2- methylpyrrolidin-1- yl)phenyl)pyridin-3-yl)- 3,4-dihydroisoquinolin- 1(2H)-one | 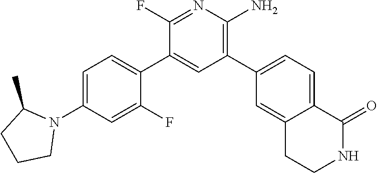 [SMILES: C[C@@H]1CCCN1c1ccc(-c2cc(-c3ccc4c(c3)CCNC4=O)c(N)nc2F)c(F)c1] | B | D |
| I-48 | (R)-6-(2-amino-6-fluoro- 5-(4-(3-isopropyl-4- methylpiperazin-1- yl)phenyl)pyridin-3-yl)- 3,4-dihydroisoquinolin- 1(2H)-one | 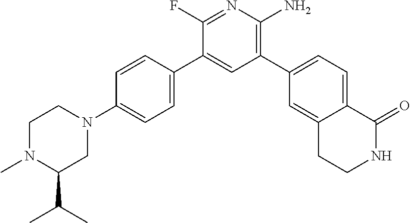 [SMILES: CC(C)[C@@H]1CN(c2ccc(-c3cc(-c4ccc5c(c4)CCNC5=O)c(N)nc3F)cc2)CCN1C] | A | D |
| I-49 | (R)-6-(2-amino-6-fluoro- 5-(2-fluoro-4-(3- isopropyl-4- methylpiperazin-1- yl)phenyl)pyridin-3-yl)- 3,4-dihydroisoquinolin- 1(2H)-one | 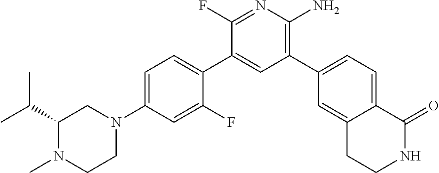 [SMILES: CC(C)[C@@H]1CN(c2ccc(-c3cc(-c4ccc5c(c4)CCNC5=O)c(N)nc3F)c(F)c2)CCN1C] | A | D |
| I-50 | (S)-6-(2-amino-6-fluoro- 5-(4-(3-isopropyl-4- methylpiperazin-1- yl)phenyl)pyridin-3-yl)- 3,4-dihydroisoquinolin- 1(2H)-one | 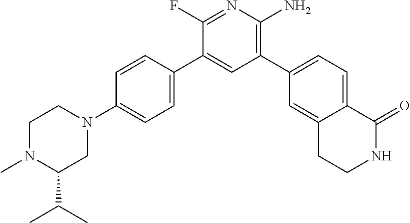 [SMILES: CC(C)[C@H]1CN(c2ccc(-c3cc(-c4ccc5c(c4)CCNC5=O)c(N)nc3F)cc2)CCN1C] | A | D |
| I-51 | (S)-6-(2-amino-6-fluoro- 5-(2-fluoro-4-(3- isopropyl-4- methylpiperazin-1- yl)phenyl)pyridin-3-yl)- 3,4-dihydroisoquinolin- 1(2H)-one | 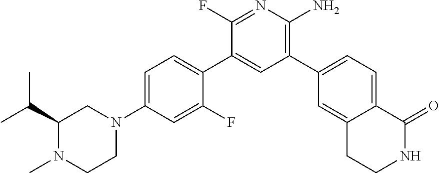 [SMILES: CC(C)[C@H]1CN(c2ccc(-c3cc(-c4ccc5c(c4)CCNC5=O)c(N)nc3F)c(F)c2)CCN1C] | A | D |
| I-52 | 6-(2-amino-5-(4-(1- ethylpiperidin-4- yl)phenyl)-6- fluoropyridin-3-yl)-3,4- dihydroisoquinolin- 1(2H)-one | 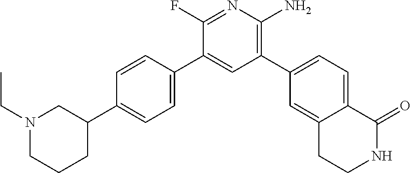 [SMILES: CCN1CCCC(c2ccc(-c3cc(-c4ccc5c(c4)CCNC5=O)c(N)nc3F)cc2)C1] | A | D |
| I-53 | 6-(2-amino-5-(4-(1- ethylpiperidin-4- yl)phenyl)-6- fluoropyridin-3-yl)-3,4- dihydroisoquinolin- 1(2H)-one | 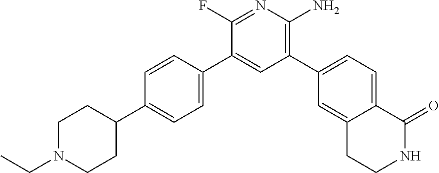 [SMILES: CCN1CCC(c2ccc(-c3cc(-c4ccc5c(c4)CCNC5=O)c(N)nc3F)cc2)CC1] | A | D |
| I-54 | 6-(2-amino-6-fluoro-5-(4- ((1- (methylsulfonyl)piperidin- 4-yl)oxy)phenyl)pyridin- 3-yl)-3,4- dihydroisoquinolin- 1(2H)-one | 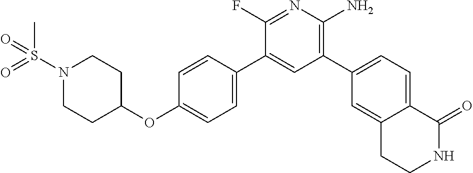 [SMILES: CS(=O)(=O)N1CCC(Oc2ccc(-c3cc(-c4ccc5c(c4)CCNC5=O)c(N)nc3F)cc2)CC1] | A | D |
| I-55 | 6-(2-amino-6-fluoro-5-(1- (tetrahydro-2H-pyran-4- yl)-1H-pyrazol-4- yl)pyridin-3-yl)-3,4- dihydroisoquinolin- 1(2H)-one | 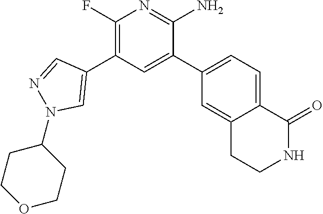 [SMILES: Nc1nc(F)c(-c2cnn(C3CCOCC3)c2)cc1-c1ccc2c(c1)CCNC2=O] | A | D |
| I-56 | tert-butyl 4-(4-(6-amino- 2-fluoro-5-(1-oxo- 1,2,3,4- tetrahydroisoquinolin-6- yl)pyridin-3-yl)phenyl)- 3,6-dihydropyridine- 1(2H)-carboxylate | 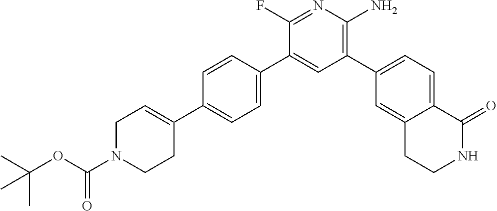 [SMILES: CC(C)(C)OC(=O)N1CC=C(c2ccc(-c3cc(-c4ccc5c(c4)CCNC5=O)c(N)nc3F)cc2)CC1] | B | D |
| I-57 | 6-(2-amino-6-fluoro-5-(4- (1,2,3,6- tetrahydropyridin-4- yl)phenyl)pyridin-3-yl)- 3,4-dihydroisoquinolin- 1(2H)-one 2,2,2- trifluoroacetate | 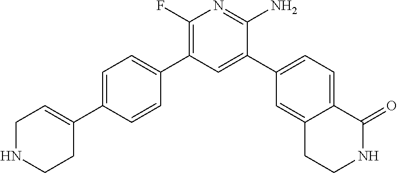 [SMILES: Nc1nc(F)c(-c2ccc(C3=CCNCC3)cc2)cc1-c1ccc2c(c1)CCNC2=O] | A | D |
| I-58 | 6-(2-amion-6-fluoro-5-(4- methyl-3,4-dihydro-2H- benzo[b][1,4]oxazin-7- yl)pyridin-3-yl)-3,4- dihydroisoquinolin- 1(2H)-one | 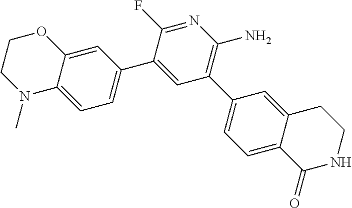 [SMILES: CN1CCOc2cc(-c3cc(-c4ccc5c(c4)CCNC5=O)c(N)nc3F)ccc21] | A | D |
| I-59 | 6-(2-amino-6-fluoro-5-(4- (pyrrolidin-1- yl)phenyl)pyridin-3-yl)- 3,4-dihydroisoquinolin- 1(2H)-one | 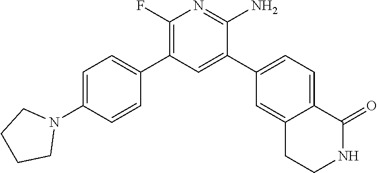 [SMILES: Nc1nc(F)c(-c2ccc(N3CCCC3)cc2)cc1-c1ccc2c(c1)CCNC2=O] | B | D |
| I-60 | 6-(2-amino-6-fluoro-5-(4- (piperidin-1- yl)phenyl)pyridin-3-yl)- 3,4-dihydroisoquinolin- 1(2H)-one | 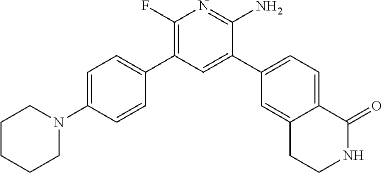 [SMILES: Nc1nc(F)c(-c2ccc(N3CCCCC3)cc2)cc1-c1ccc2c(c1)CCNC2=O] | A | D |
| I-61 | 6-(2-amino-5-(2-chloro- 4-(4-methylpiperazin-1- yl)phenyl)-6- fluoropyridin-3-yl)-3,4- dihydroisoquinolin- 1(2H)-one | 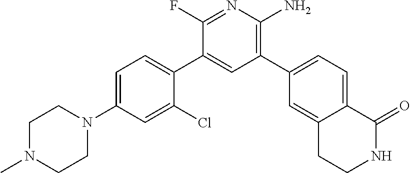 [SMILES: CN1CCN(c2ccc(-c3cc(-c4ccc5c(c4)CCNC5=O)c(N)nc3F)c(Cl)c2)CC1] | A | D |
| I-62 | 6-(2-amino-6-fluoro-5-(4- (methylsulfonyl)phenyl)py- ridin-3-yl)-3,4- dihydroisoquinolin- 1(2H)-one | 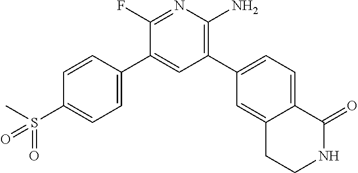 [SMILES: CS(=O)(=O)c1ccc(-c2cc(-c3ccc4c(c3)CCNC4=O)c(N)nc2F)cc1] | A | D |
| I-63 | 6-(2-amino-5-(4- (cyclopropylsulfonyl)phe- nyl)-6-fluoropyridin-3-yl)- 3,4-dihydroisoquinolin- 1(2H)-one | 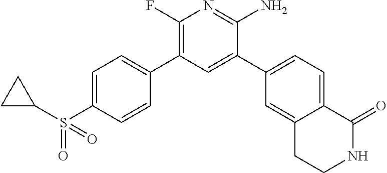 [SMILES: Nc1nc(F)c(-c2ccc(S(=O)(=O)C3CC3)cc2)cc1-c1ccc2c(c1)CCNC2=O] | A | D |
| I-64 | 4-(6-amino-2-fluoro-5-(1- oxo-1,2,3,4- tetrahydroisoquinolin-6- yl)pyridin-3-yl)-N,N- dimethylbenzenesulfona- mide | 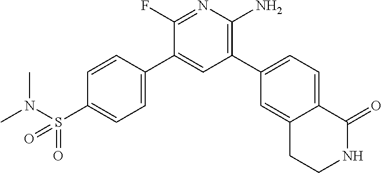 [SMILES: CN(C)S(=O)(=O)c1ccc(-c2cc(-c3ccc4c(c3)CCNC4=O)c(N)nc2F)cc1] | A | D |
| I-65 | 4-(6-amino-2-fluoro-5-(1- oxo-1,2,3,4- tetrahydroisoquinolin-6- yl)pyridin-3-yl)-N,N- diethylbenzenesulfonamide | 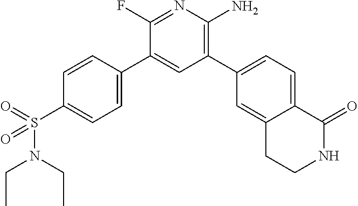 [SMILES: CCN(CC)S(=O)(=O)c1ccc(-c2cc(-c3ccc4c(c3)CCNC4=O)c(N)nc2F)cc1] | B | D |
| I-66 | 6-(2-amino-6-fluoro-5-(4- ((1R,5S)-3-(tetrahydro- 2H-pyran-4-yl)-3- azabicyclo[3.1.0]hexan- 1-yl)phenyl)pyridin-3-yl)- 3,4-dihydroisoquinolin- 1(2H)-one | 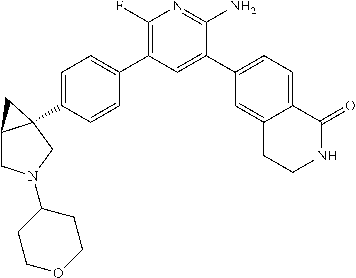 [SMILES: Nc1nc(F)c(-c2ccc([C@@]34C[C@@H]3CN(C3CCOCC3)C4)cc2)cc1-c1ccc2c(c1)CCNC2=O] | A | D |
| I-67 | 7-(2-amino-5-(4-(4- ehtylpiperazin-1- yl)phenyl)-6- fluoropyridin-3-yl)- 2,3,4,5-tetrahydro-1H- benzo[c]azepin-1-one | 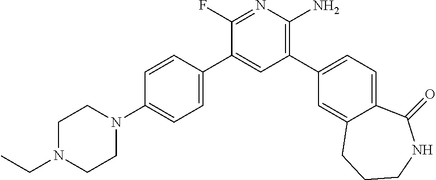 [SMILES: CCN1CCN(c2ccc(-c3cc(-c4ccc5c(c4)CCCNC5=O)c(N)nc3F)cc2)CC1] | B | D |
| I-68 | 6-(2-amino-6-fluoro-5-(4- (4-methylpiperazin-1-yl)- 3- (trifluoromethyl)phenyl)py- ridin-3-yl)-3,4- dihydroisoquinolin- 1(2H)-one | 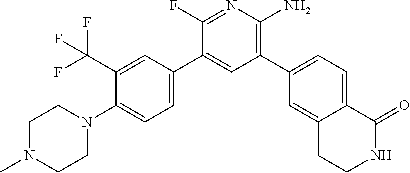 [SMILES: CN1CCN(c2ccc(-c3cc(-c4ccc5c(c4)CCNC5=O)c(N)nc3F)cc2C(F)(F)F)CC1] | A | D |
| I-69 | 6-(2-amino-5-(4-(4- ethylpiperazin-1- yl)phenyl)-6- fluoropyridin-3-yl)-4,4- dimethyl-3,4- dihydroisoquinolin- 1(2H)-one | 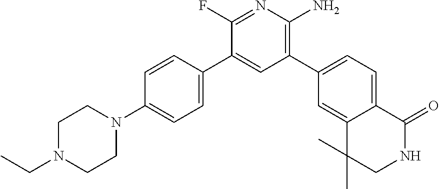 [SMILES: CCN1CCN(c2ccc(-c3cc(-c4ccc5c(c4)C(C)(C)CNC5=O)c(N)nc3F)cc2)CC1] | A | C |
| I-70 | 6-(2-amino-5-(4-(4- ethylpiperazin-1- yl)phenyl)-6- fluoropyridin-3-yl)-3,3- dimethyl-3,4- dihydroisoquinolin- 1(2H)-one 2,2,2- trifluoroacetate | 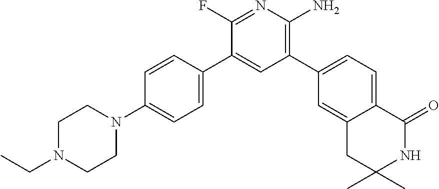 [SMILES: CCN1CCN(c2ccc(-c3cc(-c4ccc5c(c4)CC(C)(C)NC5=O)c(N)nc3F)cc2)CC1] | B | D |
| I-71 | 6-(2-amino-5-(4- ((1R,5S)-3-ethyl-3- azabicyclo[3.1.0]hexan- 1-yl)phenyl)-6- fluoropyridin-3-yl)-3,4- dihydroisoquinolin- 1(2H)-one | 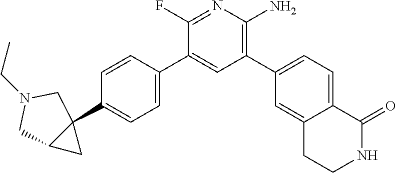 [SMILES: CCN1C[C@H]2C[C@@]2(c2ccc(-c3cc(-c4ccc5c(c4)CCNC5=O)c(N)nc3F)cc2)C1] | A | D |
| I-72 | 6-(2-amino-5-(4- ((1R,5S)-3-(2,2 difluoroethyl)-3- azabicyclo[3.1.0]hexan- 1-yl)phenyl)-6- fluoropyridin-3-yl)-3,4- dihydroisoquinolin- 1(2H)-one | 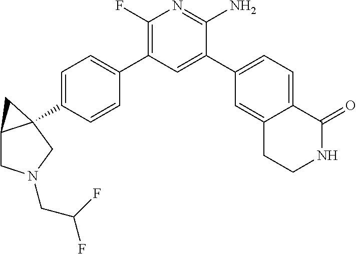 [SMILES: Nc1nc(F)c(-c2ccc([C@@]34C[C@@H]3CN(CC(F)F)C4)cc2)cc1-c1ccc2c(c1)CCNC2=O] | B | D |
| I-73 | 6-(2-amino-5-(4-(1-(2,2- difluoroethyl)-1,2,3,6- tetrahydropyridin-4- yl)phenyl)-6- fluoropyridin-3-yl)-3,4- dihydroisoquinolin- 1(2H)-one | 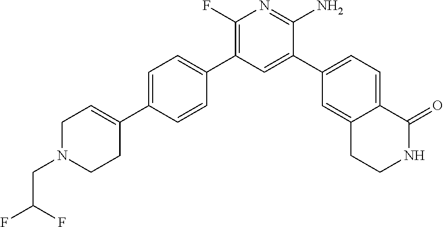 [SMILES: Nc1nc(F)c(-c2ccc(C3=CCN(CC(F)F)CC3)cc2)cc1-c1ccc2c(c1)CCNC2=O] | A | D |
| I-74 | 6-(2-amino-6-chloro-5- (4-(4-methylpiperazin-1- yl)phenyl)pyridin-3-yl)- 3,4-dihydroisoquinolin- 1(2H)-one | 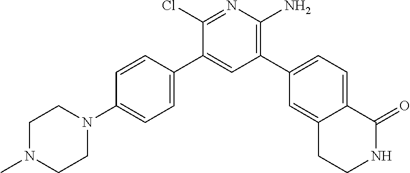 [SMILES: CN1CCN(c2ccc(-c3cc(-c4ccc5c(c4)CCNC5=O)c(N)nc3Cl)cc2)CC1] | B | D |
| I-75 | 6-(2-amino-6-chloro-5- (4-(4-ethylpiperazin-1- yl)phenyl)pyridin-3-yl)- 3,4-dihydroisoquinolin- 1(2H)-one | 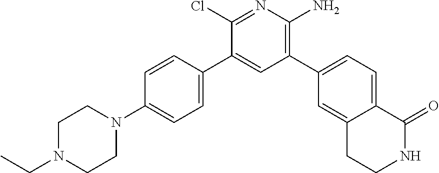 [SMILES: CCN1CCN(c2ccc(-c3cc(-c4ccc5c(c4)CCNC5=O)c(N)nc3Cl)cc2)CC1] | B | D |
| I-76 | 6-(2-amino-5-(4-(1-ethyl- 1,2,3,6- tetrahydropyridin-4- yl)phenyl)-6- fluoropyridin-3-yl)-3,4- dihydroisoquinolin- 1(2H)-one | 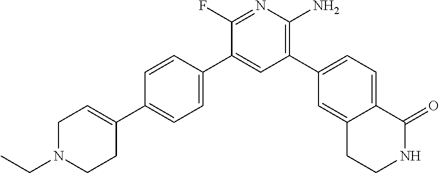 [SMILES: CCN1CC=C(c2ccc(-c3cc(-c4ccc5c(c4)CCNC5=O)c(N)nc3F)cc2)CC1] | A | D |
| I-77 | (S)-6-(2-amino-6-fluoro- 5-(4-(2- isopropylmorpholino)phe- nyl)pyridin-3-yl)-3,4- dihydro-2,7-naphthyridin- 1(2H)-one 2,2,2- trifluoroacetate | 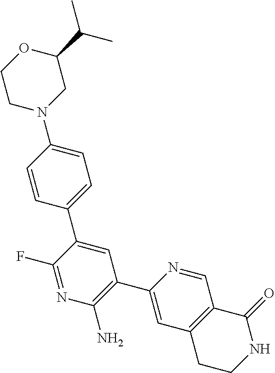 [SMILES: CC(C)[C@H]1CN(c2ccc(-c3cc(-c4cc5c(cn4)C(=O)NCC5)c(N)nc3F)cc2)CCO1] | A | D |
| I-78 | 6-(2-amino-5-(4-(1-(2,2- difluoroethyl)piperidin-3- yl)phenyl)-6- fluoropyridin-3-yl)-3,4- dihydroisoquinolin- 1(2H)-one | 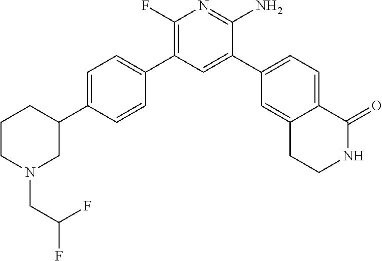 [SMILES: Nc1nc(F)c(-c2ccc(C3CCCN(CC(F)F)C3)cc2)cc1-c1ccc2c(c1)CCNC2=O] | A | D |
| I-79 | 6-(2-amino-6-fluoro-5-(4- (1-isopropylpiperidin-4- yl)phenyl)pyridin-3-yl)- 3,4-dihydroisoquinolin- 1(2H)-one | 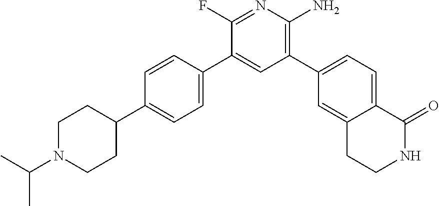 [SMILES: CC(C)N1CCC(c2ccc(-c3cc(-c4ccc5c(c4)CCNC5=O)c(N)nc3F)cc2)CC1] | A | D |
| I-80 | 6-(2-amino-6-fluoro-5-(4- (1-isopropylpiperidin-3- yl)phenyl)pyridin-3-yl)- 3,4-dihydroisoquinolin- 1(2H)-one | 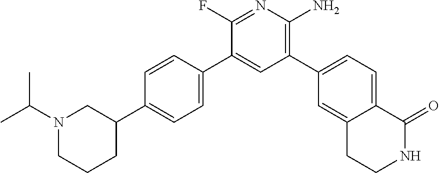 [SMILES: CC(C)N1CCCC(c2ccc(-c3cc(-c4ccc5c(c4)CCNC5=O)c(N)nc3F)cc2)C1] | A | D |
| I-81 | 6-(2-amino-6-fluoro-5-(4- (1-isopropyl-1,2,3,6- tetrahydropyridin-4- yl)phenyl)pyridin-3-yl)- 3,4-dihydroisoquinolin- 1(2H)-one | 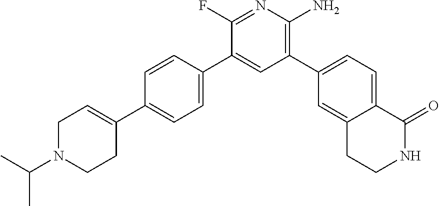 [SMILES: CC(C)N1CC=C(c2ccc(-c3cc(-c4ccc5c(c4)CCNC5=O)c(N)nc3F)cc2)CC1] | A | D |
| I-82 | 6-(2-amino-6-fluoro-5-(4- (1-(2- hydroxyethyl)piperidin-3- yl)phenyl)pyridin-3-yl)- 3,4-dihydroisoquinolin- 1(2H)-one | 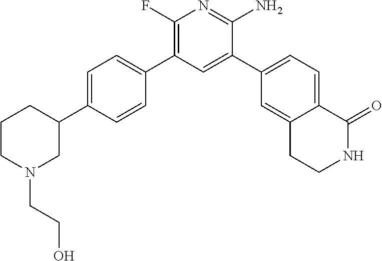 [SMILES: Nc1nc(F)c(-c2ccc(C3CCCN(CCO)C3)cc2)cc1-c1ccc2c(c1)CCNC2=O] | A | D |
| I-83 | tert-butyl 4-((4-(6-amino- 2-fluoro-5-(1-oxo- 1,2,3,4- tetrahydroisoquinolin-6- yl)pyridin-3- yl)phenyl)sulfonyl)piperidine- 1-carboxylate | 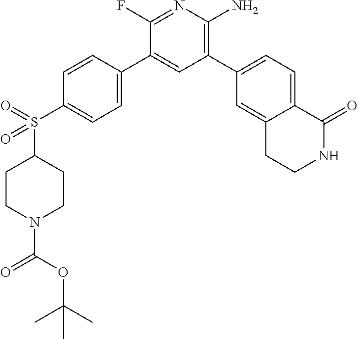 [SMILES: CC(C)(C)OC(=O)N1CCC(S(=O)(=O)c2ccc(-c3cc(-c4ccc5c(c4)CCNC5=O)c(N)nc3F)cc2)CC1] | B | D |
| I-84 | 6-(2-amino-5-(4- (azetidin-3- ylsulfonyl)phenyl)-6- fluoropyridin-3-yl)-3,4- dihydroisoquinolin- 1(2H)-one | 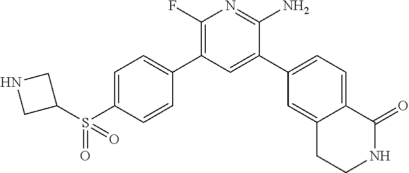 [SMILES: Nc1nc(F)c(-c2ccc(S(=O)(=O)C3CNC3)cc2)cc1-c1ccc2c(c1)CCNC2=O] | B | D |
| I-85 | 6-(2-amino-5-(4- (ethylsulfonimidoyl)phenyl)- 6-fluoropyridin-3-yl)- 3,4-dihydroisoquinolin- 1(2H)-one | 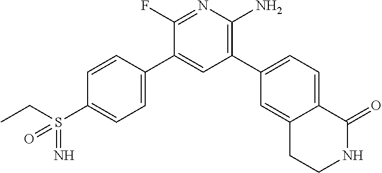 [SMILES: CCS(=N)(=O)c1ccc(-c2cc(-c3ccc4c(c3)CCNC4=O)c(N)nc2F)cc1] | B | D |
| I-86 | 6-(2-amino-6-fluoro-5-(4- (S- methylsulfonimidoyl)phe- nyl)pyridin-3-yl)-3,4- dihydroisoquinolin- 1(2H)-one | 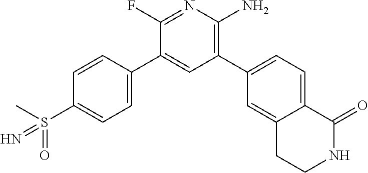 [SMILES: CS(=N)(=O)c1ccc(-c2cc(-c3ccc4c(c3)CCNC4=O)c(N)nc2F)cc1] | A | D |
| I-87 | 6-(2-amino-6-fluoro-5-(4- ((trifluoromethyl)sulfonyl) phenyl)pyridin-3-yl)-3,4- dihydroisoquinolin- 1(2H)-one | 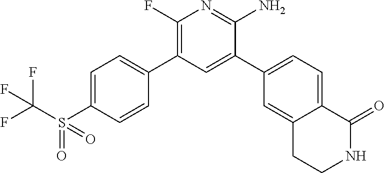 [SMILES: Nc1nc(F)c(-c2ccc(S(=O)(=O)C(F)(F)F)cc2)cc1-c1ccc2c(c1)CCNC2=O] | B | D |
| I-88 | 6-(2-amino-5-(4-(3,4- dimethylpiperazin-1- yl)phenyl)-6- fluoropyridin-3-yl)-3,4- dihydroisoquinolin- 1(2H)-one | 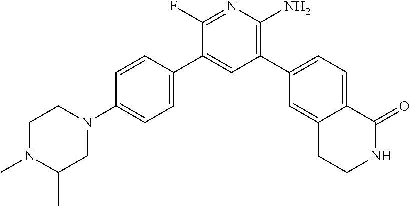 [SMILES: CC1CN(c2ccc(-c3cc(-c4ccc5c(c4)CCNC5=O)c(N)nc3F)cc2)CCN1C] | A | D |
| I-89 | 6-(2-amino-6-fluoro-5-(4- (1-(2,2,2- trifluoroethyl)piperidin-3- yl)phenyl)pyridin-3-yl)- 3,4-dihydroisoquinolin- 1(2H)-one | 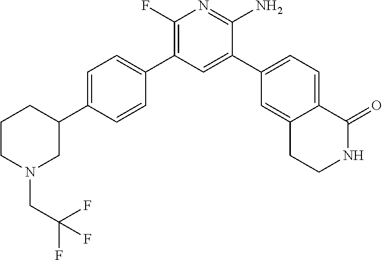 [SMILES: Nc1nc(F)c(-c2ccc(C3CCCN(CC(F)(F)F)C3)cc2)cc1-c1ccc2c(c1)CCNC2=O] | B | D |
| I-90 | 6-(2-amino-6-fluoro-5-(4- (4-(2,2,2- trifluoroethyl)morpholin- 2-yl)phenyl)pyridin-3-yl)- 3,4-dihydroisoquinolin- 1(2H)-one | 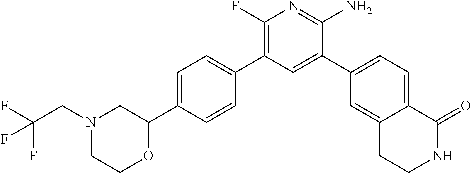 [SMILES: Nc1nc(F)c(-c2ccc(C3CN(CC(F)(F)F)CCO3)cc2)cc1-c1ccc2c(c1)CCNC2=O] | B | D |
| I-91 | 6-(2-amino-6-fluoro-5-(4- ((3aR,6aS)-tetrahydro- 1H-furo[3,4-c]pyrrol- 5(3H)-yl)phenyl)pyridin- 3-yl)-3,4- dihydroisoquinolin- 1(2H)-one | 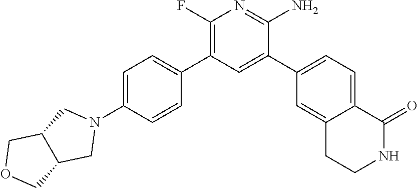 [SMILES: Nc1nc(F)c(-c2ccc(N3C[C@H]4COC[C@H]4C3)cc2)cc1-c1ccc2c(c1)CCNC2=O] | B | D |
| I-92 | 6-(2-amino-6-fluoro-5-(4- ((3aS,6aS)-hexahydro- 5H-furo[2,3-c]pyrrol-5- yl)phenyl)pyridin-3-yl)- 3,4-dihydroisoquinolin- 1(2H)-one | 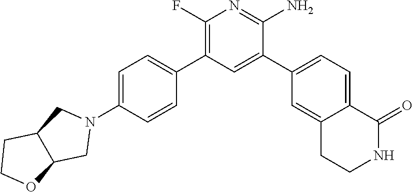 [SMILES: Nc1nc(F)c(-c2ccc(N3C[C@@H]4CCO[C@@H]4C3)cc2)cc1-c1ccc2c(c1)CCNC2=O] | A | D |
| I-93 | 6-(2-amino-6-fluoro-5-(4- (1-(2,2,2- trifluoroethyl)piperidin-2- yl)phenyl)pyridin-3-yl)- 3,4-dihydroisoquinolin- 1(2H)-one | 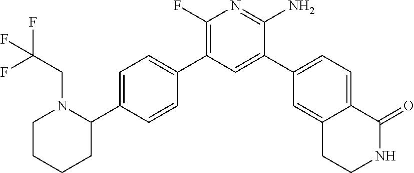 [SMILES: Nc1nc(F)c(-c2ccc(C3CCCCN3CC(F)(F)F)cc2)cc1-c1ccc2c(c1)CCNC2=O] | B | D |
| I-94 | (S)-6-(2-amino-6-fluoro- 5-(4-(piperidin-3- yloxy)phenyl)pyridin-3- yl)-3,4- dihydroisoquinolin- 1(2H)-one | 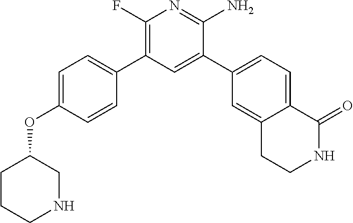 [SMILES: Nc1nc(F)c(-c2ccc(O[C@H]3CCCNC3)cc2)cc1-c1ccc2c(c1)CCNC2=O] | A | D |
| I-95 | 6-(2-amino-6-fluoro-5-(4- (4-isopropylpiperazin-1- yl)phenyl)pyridin-3-yl)-7- fluoro-3,4- dihydroisoquinolin- 1(2H)-one | 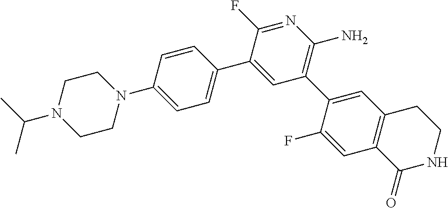 [SMILES: CC(C)N1CCN(c2ccc(-c3cc(-c4cc5c(cc4F)C(=O)NCC5)c(N)nc3F)cc2)CC1] | A | D |
| I-96 | 6-(2-amino-6-fluoro-5-(4- ((3R,5S)-3,4,5- trimethylpiperazin-1- yl)phenyl)pyridin-3-yl)- 3,4-dihydroisoquinolin- 1(2H)-one | 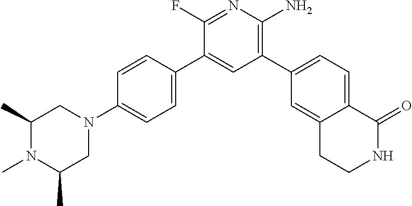 [SMILES: C[C@@H]1CN(c2ccc(-c3cc(-c4ccc5c(c4)CCNC5=O)c(N)nc3F)cc2)C[C@H](C)N1C] | A | D |
| I-97 | 6-(2-amino-6-fluoro-5-(4- ((3S,5S)-3,4,5- trimethylpiperazin-1- yl)phenyl)pyridin-3-yl)- 3,4-dihydroisoquinolin- 1(2H)-one | 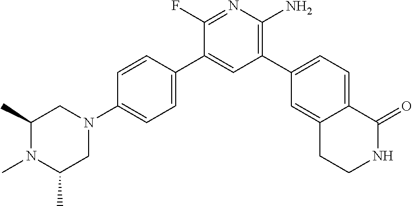 [SMILES: C[C@H]1CN(c2ccc(-c3cc(-c4ccc5c(c4)CCNC5=O)c(N)nc3F)cc2)C[C@H](C)N1C] | A | D |
| I-98 | 6-(2-amino-6-fluoro-5-(4- (morpholinosulfonyl)phe- nyl)pyridin-3-yl)-3,4- dihydroisoquinolin- 1(2H)-one | 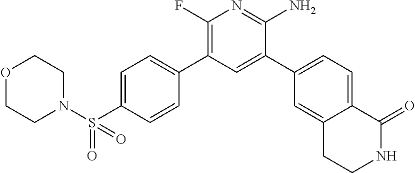 [SMILES: Nc1nc(F)c(-c2ccc(S(=O)(=O)N3CCOCC3)cc2)cc1-c1ccc2c(c1)CCNC2=O] | B | D |
| I-99 | 6-(2-amino-6-fluoro-5-(4- (pyrrolidin-1- ylsulfonyl)phenyl)pyridin- 3-yl)-3,4- dihydroisoquinolin- 1(2H)-one | 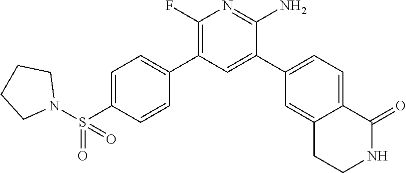 [SMILES: Nc1nc(F)c(-c2ccc(S(=O)(=O)N3CCCC3)cc2)cc1-c1ccc2c(c1)CCNC2=O] | B | D |
| I-100 | 6-(2-amino-5-(4-(1- ethylpiperidin-2- yl)phenyl)-6- fluoropyridin-3-yl-3,4- dihydroisoquinolin- 1(2H)-one | 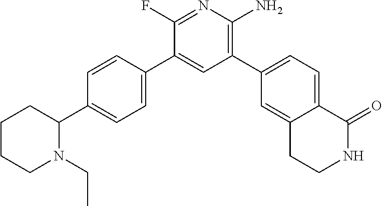 [SMILES: CCN1CCCCC1c1ccc(-c2cc(-c3ccc4c(c3)CCNC4=O)c(N)nc2F)cc1] | A | D |
| I-101 | 6-(2-amino-5-(4-(1-(2,2- difluoroethyl)piperidin-2- yl)phenyl)-6- fluoropyridin-3-yl)-3,4- dihydroisoquinolin- 1(2H)-one | 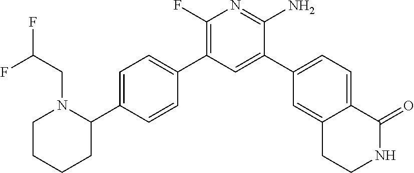 [SMILES: Nc1nc(F)c(-c2ccc(C3CCCCN3CC(F)F)cc2)cc1-c1ccc2c(c1)CCNC2=O] | B | D |
| I-102 | 6-(2-amino-5-(4-(4- ethylmorpholin-2- yl)phenyl)-6- fluoropyridin-3-yl)-3,4- dihydroisoquinolin- 1(2H)-one | 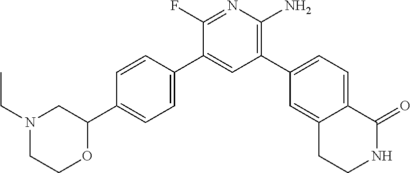 [SMILES: CCN1CCOC(c2ccc(-c3cc(-c4ccc5c(c4)CCNC5=O)c(N)nc3F)cc2)C1] | B | D |
| I-103 | 6-(2-amino-5-(4-(4-(2,2- difluoroethyl)morpholin- 2-yl)phenyl)-6- fluoropyriidn-3-yl)-3,4- dihydroisoquinolin- 1(2H)-one | 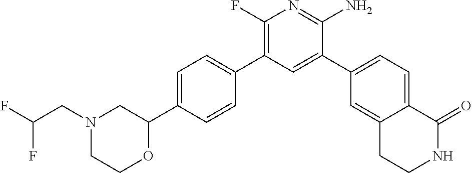 [SMILES: Nc1nc(F)c(-c2ccc(C3CN(CC(F)F)CCO3)cc2)cc1-c1ccc2c(c1)CCNC2=O] | B | D |
| I-104 | 6-(2-amino-6-fluoro-5-(4- (1-isopropylpiperidin-2- yl)phenyl)pyridin-3-yl)- 3,4-dihydroisoquinolin- 1(2H)-one | 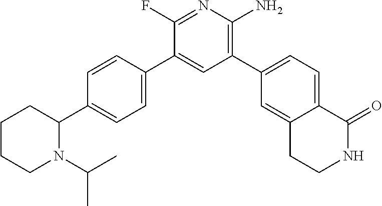 [SMILES: CC(C)N1CCCCC1c1ccc(-c2cc(-c3ccc4c(c3)CCNC4=O)c(N)nc2F)cc1] | A | D |
| I-105 | 6-(2-amino-6-fluoro-5-(4- (4-isopropylmorpholin-2- yl)phenyl)pyridin-3-yl)- 3,4-dihydroisoquinolin- 1(2H)-one | 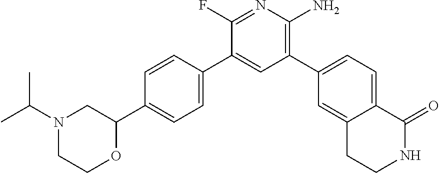 [SMILES: CC(C)N1CCOC(c2ccc(-c3cc(-c4ccc5c(c4)CCNC5=O)c(N)nc3F)cc2)C1] | A | D |
| I-106 | 6-(2-amino-6-fluoro-5-(4- ((1S,4R)-3-methyl-3- azabicyclo[3.1.0]hexan- 1-yl)phenyl)pyridin-3-yl)- 3,4-dihydroisoquinolin- 1(2H)-one | 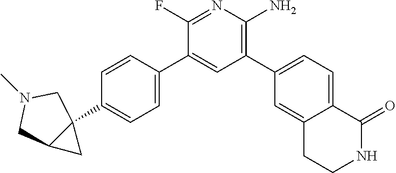 [SMILES: CN1C[C@@H]2C[C@]2(c2ccc(-c3cc(-c4ccc5c(c4)CCNC5=O)c(N)nc3F)cc2)C1] | A | D |
| I-107 | 6-(2-amino-6-fluoro-5-(4- (1-(2- methoxyethyl)piperidin- 4-yl)phenyl)pyridin-3-yl)- 3,4-dihydroisoquinolin- 1(2H)-one | 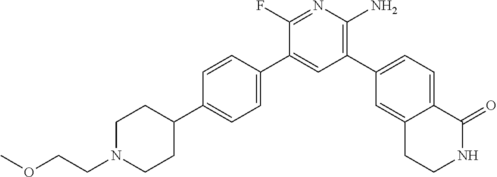 [SMILES: COCCN1CCC(c2ccc(-c3cc(-c4ccc5c(c4)CCNC5=O)c(N)nc3F)cc2)CC1] | A | D |
| I-108 | (R)-6-(2-amino-6-fluoro- 5-(2-fluoro-4-(3- methylmorpholino)phenyl) pyridin-3-yl)-3,4- dihydroisoquinolin- 1(2H)-one | 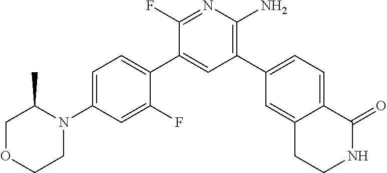 [SMILES: C[C@@H]1COCCN1c1ccc(-c2cc(-c3ccc4c(c3)CCNC4=O)c(N)nc2F)c(F)c1] | B | D |
| I-109 | 6-(2-amino-5-(4-(2,5- dihydro-1H-pyrrol-3- yl)phenyl)-6- fluoropyridin-3-yl)-3,4- dihydroisoquinolin- 1(2H)-one 2,2,2- trifluoroacetate | 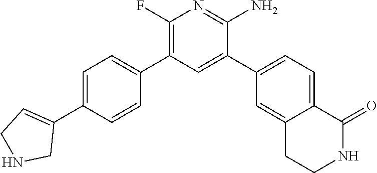 [SMILES: Nc1nc(F)c(-c2ccc(C3=CCNC3)cc2)cc1-c1ccc2c(c1)CCNC2=O] | A | D |
| I-110 | 6-(2-amino-6-fluoro-5-(4- ((1R,5S)-3-isopropyl-3- azabicyclo[3.1.0]hexan- 1-yl)phenyl)pyridin-3-yl)- 3,4-dihydroisoquinolin- 1(2H)-one | 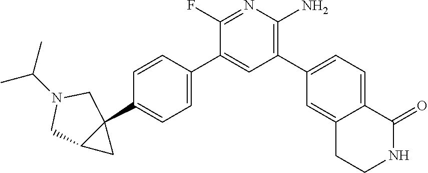 [SMILES: CC(C)N1C[C@H]2C[C@@]2(c2ccc(-c3cc(-c4ccc5c(c4)CCNC5=O)c(N)nc3F)cc2)C1] | A | D |
| I-111 | 6-(2-amino-6-fluoro-5-(4- ((1S,5R)-3-isopropyl-3- azabicyclo[3.1.0]hexan- 1-yl)phenyl)pyridin-3-yl)- 3,4-dihydroisoquinolin- 1(2H)-one 2,2,2- trifluoroacetate | 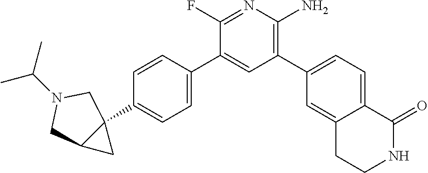 [SMILES: CC(C)N1C[C@@H]2C[C@]2(c2ccc(-c3cc(-c4ccc5c(c4)CCNC5=O)c(N)nc3F)cc2)C1] | A | D |
| I-112 | (R)-6-(2-amino-6-fluoro- 5-(4-(3-fluoropyrrolidin-1- yl)phenyl)pyridin-3-yl)- 3,4-dihydroisoquinolin- 1(2H)-one | 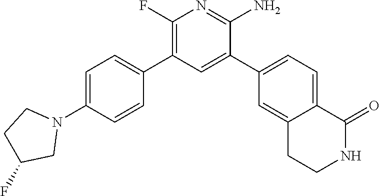 [SMILES: Nc1nc(F)c(-c2ccc(N3CC[C@@H](F)C3)cc2)cc1-c1ccc2c(c1)CCNC2=O] | B | D |
| I-113 | (S)-6-(2-amino-6-fluoro- 5-(4-(3-fluoropyrrolidin-1- yl)phenyl)pyridin-3-yl)- 3,4-dihydroisoquinolin- 1(2H)-one | 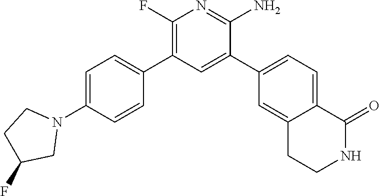 [SMILES: Nc1nc(F)c(-c2ccc(N3CC[C@H](F)C3)cc2)cc1-c1ccc2c(c1)CCNC2=O] | B | D |
| I-114 | (R)-6-(2-amino-6-fluoro- 5-(4-(3- methoxypyrrolidin-1- yl)phenyl)pyridin-3-yl)- 3,4-dihydroisoquinolin- 1(2H)-one | 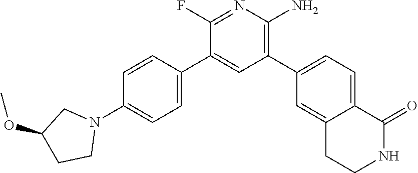 [SMILES: CO[C@@H]1CCN(c2ccc(-c3cc(-c4ccc5c(c4)CCNC5=O)c(N)nc3F)cc2)C1] | B | D |
| I-115 | (S)-6-(2-amino-6-fluoro- 5-(4-(3- methoxypyrrolidin-1- yl)phenyl)pyridin-3-yl)- 3,4-dihydroisoquinolin- 1(2H)-one | 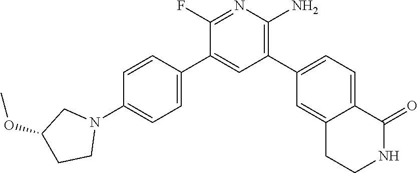 [SMILES: CO[C@H]1CCN(c2ccc(-c3cc(-c4ccc5c(c4)CCNC5=O)c(N)nc3F)cc2)C1] | A | D |
| I-116 | (R)-6-(2-amino-6-fluoro- 5-(4-(3- (methylsulfonyl)pyrrolidin- 1-yl)phenyl)pyridin-3-yl)- 3,4-dihydroisoquinolin- 1(2H)-one | 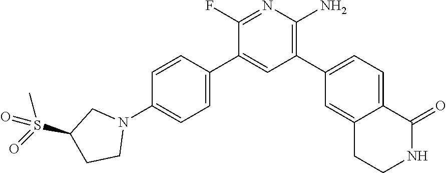 [SMILES: CS(=O)(=O)[C@@H]1CCN(c2ccc(-c3cc(-c4ccc5c(c4)CCNC5=O)c(N)nc3F)cc2)C1] | A | D |
| I-117 | (S)-6-(2-amino-6-fluoro- 5-(4-(3- (methylsulfonyl)pyrrolidin- 1-yl)phenyl)pyridin-3-yl)- 3,4-dihydroisoquinolin- 1(2H)-one | 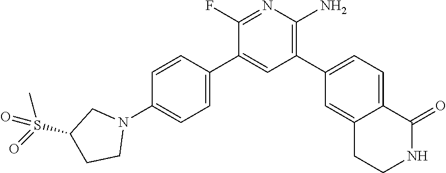 [SMILES: CS(=O)(=O)[C@H]1CCN(c2ccc(-c3cc(-c4ccc5c(c4)CCNC5=O)c(N)nc3F)cc2)C1] | A | D |
| I-118 | 6-(2-amino-6-fluoro-5-(4- (4-methyl-5,6-dihydro- 1,2,4-triazin-1(4H)- yl)phenyl)pyridin-3-yl)- 3,4-dihydroisoquinolin- 1(2H)-one | 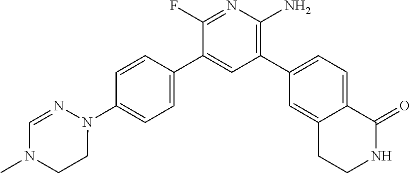 [SMILES: CN1C=NN(c2ccc(-c3cc(-c4ccc5c(c4)CCNC5=O)c(N)nc3F)cc2)CC1] | A | D |
| I-119 | 6-(2-amino-6-fluoro-5-(4- (1-(2- hydroxyethyl)piperidin-4- yl)phenyl)pyridin-3-yl)- 3,4-dihydroisoquinolin- 1(2H)-one | 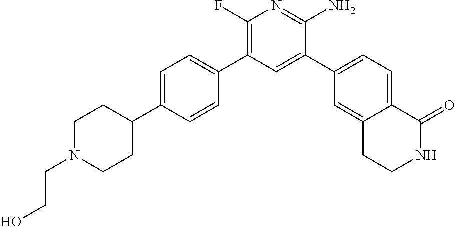 [SMILES: Nc1nc(F)c(-c2ccc(C3CCN(CCO)CC3)cc2)cc1-c1ccc2c(c1)CCNC2=O] | A | D |
| I-120 | 6-(2-amino-5-(4-(1-ethyl- 2,5-dihydro-1H-pyrrol-3- yl)phenyl)-6- fluoropyridin-3-yl)-3,4- dihydroisoquinolin- 1(2H)-one | 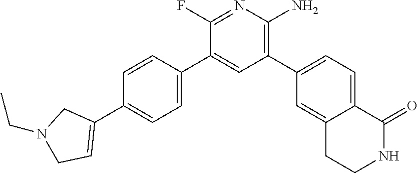 [SMILES: CCN1CC=C(c2ccc(-c3cc(-c4ccc5c(c4)CCNC5=O)c(N)nc3F)cc2)C1] | A | D |
| I-121 | 6-(2-amino-6-fluoro-5-(4- (1-isopropyl-2,5-dihydro- 1H-pyrrol-3- yl)phenyl)pyridin-3-yl)- 3,4-dihydroisoquinolin- 1(2H)one | 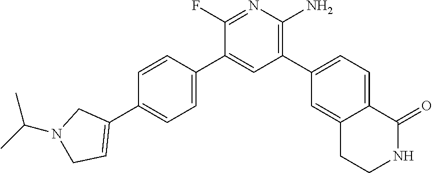 [SMILES: CC(C)N1CC=C(c2ccc(-c3cc(-c4ccc5c(c4)CCNC5=O)c(N)nc3F)cc2)C1] | A | D |
| I-122 | 6-(2-amino-6-fluoro-5-(4- (1-(2- methoxyethyl)piperidin- 3-yl)phenyl)pyridin-3-yl)- 3,4-dihydroisoquinolin- 1(2H)-one | 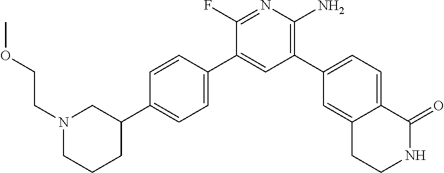 [SMILES: COCCN1CCCC(c2ccc(-c3cc(-c4ccc5c(c4)CCNC5=O)c(N)nc3F)cc2)C1] | A | D |
| I-123 | 6-(2-amino-6-fluoro-5-(4- (1-(2-hydroxyethyl)-2,5- dihydro-1H-pyrrol-3- yl)phenyl)pyridin-3-yl)- 3,4-dihydroisoquinolin- 1(2H)-one | 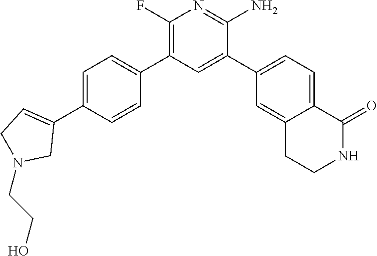 [SMILES: Nc1nc(F)c(-c2ccc(C3=CCN(CCO)C3)cc2)cc1-c1ccc2c(c1)CCNC2=O] | A | D |
| I-124 | 6-(2-amino-6-fluoro-5-(4- (1-(2-methoxyethyl)-2,5- dihydro-1H-pyrrol-3- yl)phenyl)pyridin-3-yl)- 3,4-dihydroisoquinolin- 1(2H)-one | 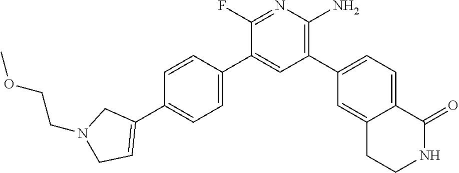 [SMILES: COCCN1CC=C(c2ccc(-c3cc(-c4ccc5c(c4)CCNC5=O)c(N)nc3F)cc2)C1] | A | D |
| I-125 | 6-(2-amino-6-fluoro-5-(4- ((1R,5S)-3-(2-hydroxy-2- methylpropyl)-3- azabicyclo[3.1.0]hexan- 1-yl)phenyl)pyridin-3-yl)- 3,4-dihydroisoquinolin- 1(2H)-one | 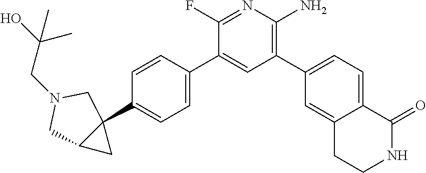 [SMILES: CC(C)(O)CN1C[C@H]2C[C@@]2(c2ccc(-c3cc(-c4ccc5c(c4)CCNC5=O)c(N)nc3F)cc2)C1] | A | D |
| I-126 | 6-(2-amino-6-fluoro-5-(4- ((1S,5R)-3-(2-hydroxy-2- methylpropyl)-3- azabicyclo[3.1.0]hexan- 1-yl)phenyl)pyridin-3-yl)- 3,4-dihydroisoquinolin- 1(2H)-one | 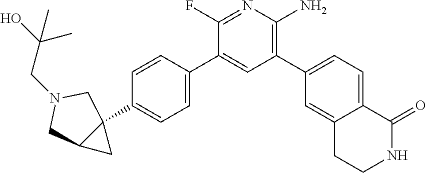 [SMILES: CC(C)(O)CN1C[C@@H]2C[C@]2(c2ccc(-c3cc(-c4ccc5c(c4)CCNC5=O)c(N)nc3F)cc2)C1] | A | D |
| I-127 | 6-(2-amino-5-(4-(1-(2,2- difluoroethyl)-2,5- dihydro-1H-pyrrol-3- yl)phenyl)-6- fluoropyridin-3-yl)-3,4- dihydroisoquinolin- 1(2H)-one | 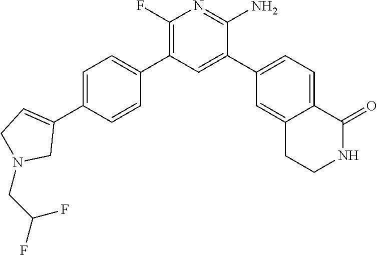 [SMILES: Nc1nc(F)c(-c2ccc(C3=CCN(CC(F)F)C3)cc2)cc1-c1ccc2c(c1)CCNC2=O] | A | D |
| I-128 | 6-(2-amino-6-fluoro-5-(4- (piperidin-4- ylsulfonyl)phenyl)pyridin- 3-yl)-3,4- dihydroisoquinolin- 1(2H)-one | 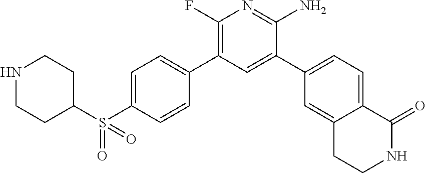 [SMILES: Nc1nc(F)c(-c2ccc(S(=O)(=O)C3CCNCC3)cc2)cc1-c1ccc2c(c1)CCNC2=O] | B | D |
| I-129 | 6-(2-amino-6-fluoro-5-(1- (1-methylpiperidin-4-yl)- 1H-pyrazol-4-yl)pyridin- 3-yl)-7-fluoro-3,4- dihydroisoquinolin- 1(2H)-one | 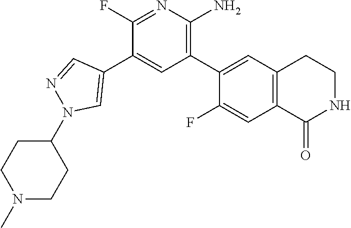 [SMILES: CN1CCC(n2cc(-c3cc(-c4cc5c(cc4F)C(=O)NCC5)c(N)nc3F)cn2)CC1] | A | C |
| I-130 | (S)-6-(2-amino-6-fluoro- 5-(4-(2- isopropylmorpholino)-3- (trifluoromethyl)phenyl)py- ridin-3-yl)-3,4- dihydroisoquinolin- 1(2H)-one | 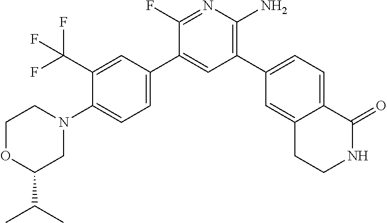 [SMILES: CC(C)[C@H]1CN(c2ccc(-c3cc(-c4ccc5c(c4)CCNC5=O)c(N)nc3F)cc2C(F)(F)F)CCO1] | B | D |
| I-131 | 6-(2-amino-5-(4-(4- ethylmorpholin-3- yl)phenyl)-6- fluoropyridin-3-yl)-3,4- dihydroisoquinolin- 1(2H)-one | 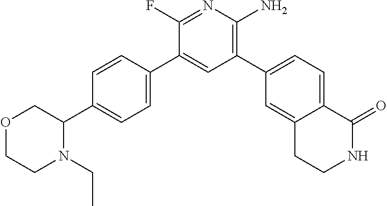 [SMILES: CCN1CCOCC1c1ccc(-c2cc(-c3ccc4c(c3)CCNC4=O)c(N)nc2F)cc1] | A | D |
| I-132 | 6-(2-amino-6-fluoro-5-(4- (4-isopropylmorpholin-3- yl)phenyl)pyridin-3-yl)- 3,4-dihydroisoquinolin- 1(2H)-one | 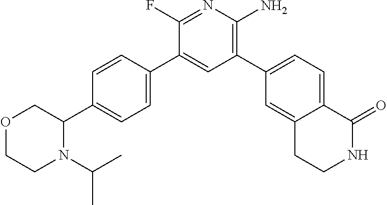 [SMILES: CC(C)N1CCOCC1c1ccc(-c2cc(-c3ccc4c(c3)CCNC4=O)c(N)nc2F)cc1] | A | D |
| I-133 | 6-(2-amino-5-(4-(4-(2,2- difluoroethyl)morpholin- 3-yl)phenyl)-6- fluoropyridin-3-yl)-3,4- dihydroisoquinolin- 1(2H)-one | 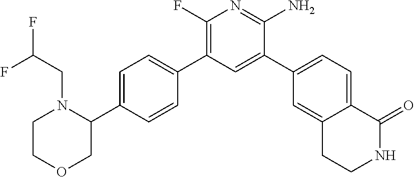 [SMILES: Nc1nc(F)c(-c2ccc(C3COCCN3CC(F)F)cc2)cc1-c1ccc2c(c1)CCNC2=O] | A | D |
| I-134 | 6-(2-amino-6-fluoro-5-(4- (4-(2- methoxyethyl)morpholin- 3-yl)phenyl)pyridin-3-yl)- 3,4-dihydroisoquinolin- 1(2H)-one | 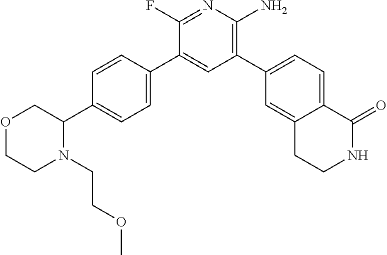 [SMILES: COCCN1CCOCC1c1ccc(-c2cc(-c3ccc4c(c3)CCNC4=O)c(N)nc2F)cc1] | A | D |
| I-135 | 6-(5-(4-((1R,5S)-3- azabicyclo[3.1.0]hexan- 1-yl)phenyl)-2-amino-6- fluoropyridin-3-yl)-3,4- dihydroisoquinolin- 1(2H)-one | 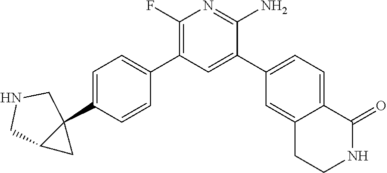 [SMILES: Nc1nc(F)c(-c2ccc([C@]34CNC[C@H]3C4)cc2)cc1-c1ccc2c(c1)CCNC2=O] | A | D |
| I-136 | (S)-6-(2-amino-6-chloro- 5-(4-(2- isopropylmorpholino)phe- nyl)pyridin-3-yl)-3,4- dihydroisoquinolin- 1(2H)-one | 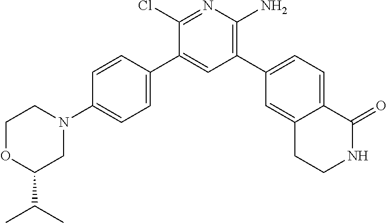 [SMILES: CC(C)[C@H]1CN(c2ccc(-c3cc(-c4ccc5c(c4)CCNC5=O)c(N)nc3Cl)cc2)CCO1] | C | D |
| I-137 | (R)-6-(2-amino-6-chloro- 5-(2-fluoro-4-(2- isopropylmorpholino)phe- nyl)pyridin-3-yl)-3,4- dihydroisoquinolin- 1(2H)-one | 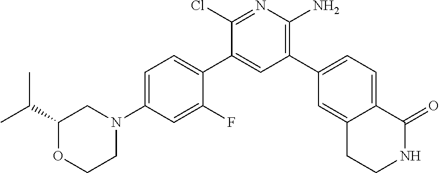 [SMILES: CC(C)[C@@H]1CN(c2ccc(-c3cc(-c4ccc5c(c4)CCNC5=O)c(N)nc3Cl)c(F)c2)CCO1] | B | D |
| I-138 | 6-(2-amino-6-fluoro-5-(4- (1-(2-hydroxyethyl)- 1,2,3,6- tetrahydropyridin-4- yl)phenyl)pyridin-3-yl)- 3,4-dihydroisoquinolin- 1(2H)-one | 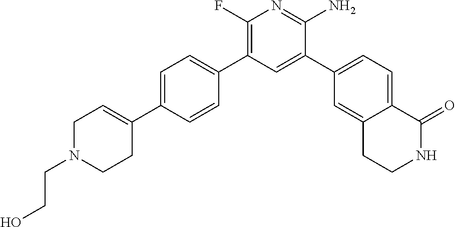 [SMILES: Nc1nc(F)c(-c2ccc(C3=CCN(CCO)CC3)cc2)cc1-c1ccc2c(c1)CCNC2=O] | A | D |
| I-139 | 6-(2-amino-6-fluoro-5-(4- (1-(2-methoxyethyl)- 1,2,3,6- tetrahydropyridin-4- yl)phenyl)pyridin-3-yl)- 3,4-dihydroisoquinolin- 1(2H)-one | 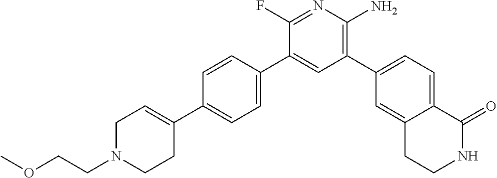 [SMILES: COCCN1CC=C(c2ccc(-c3cc(-c4ccc5c(c4)CCNC5=O)c(N)nc3F)cc2)CC1] | A | D |
| I-140 | 6-(2-amino-6-fluoro-5-(4- ((1R,5S)-3-(2- methoxyethyl)-3- azabicyclo[3.1.0]hexan- 1-yl)phenyl)pyridin-3-yl)- 3,4-dihydroisoquinolin- 1(2H)-one | 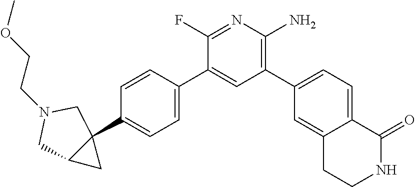 [SMILES: COCCN1C[C@H]2C[C@@]2(c2ccc(-c3cc(-c4ccc5c(c4)CCNC5=O)c(N)nc3F)cc2)C1] | A | D |
| I-141 | 6-(2-amino-6-fluoro-5-(4- ((1R,5S)-3-isopopyl-3- azabicyclo[3.1.0]hexan- 1-yl)phenyl)pyridin-3-yl)- 7-fluoro-3,4- dihydroisoquinolin- 1(2H)-one | 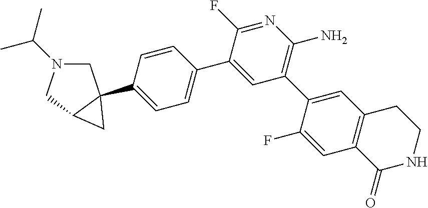 [SMILES: CC(C)N1C[C@H]2C[C@@]2(c2ccc(-c3cc(-c4cc5c(cc4F)C(=O)NCC5)c(N)nc3F)cc2)C1] | A | D |
| I-142 | 6-(2-amino-6-fluoro-5-(4- ((1S,5R)-3-isopropyl-3- azabicyclo[3.1.0]hexan- 1-yl)phenyl)pyridin-3-yl)- 7-fluoro-3,4- dihydroisoquinolin- 1(2H)-one | 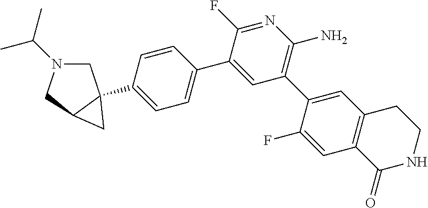 [SMILES: CC(C)N1C[C@@H]2C[C@]2(c2ccc(-c3cc(-c4cc5c(cc4F)C(=O)NCC5)c(N)nc3F)cc2)C1] | A | D |
| I-143 | 6-(2-amino-6-fluoro-5-(4- ((1R,5S)-3-isobutyryl-3- azabicyclo[3.1.0]hexan- 1-yl)phenyl)pyridin-3-yl)- 3,4-dihydroisoquinolin- 1(2H)-one | 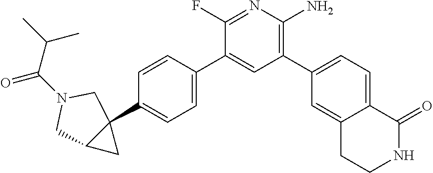 [SMILES: CC(C)C(=O)N1C[C@H]2C[C@@]2(c2ccc(-c3cc(-c4ccc5c(c4)CCNC5=O)c(N)nc3F)cc2)C1] | A | D |
| I-144 | 6-(2-amino-5-(4- (((2S,6S)-2,6- dimethylpiperidin-4- yl)oxy)phenyl)-6- fluoropyridin-3-yl)-3,4- dihydroisoquinolin- 1(2H)-one | 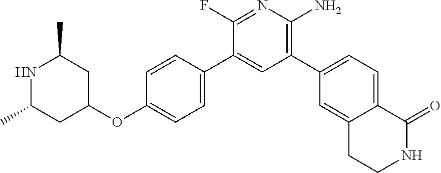 [SMILES: C[C@H]1CC(Oc2ccc(-c3cc(-c4ccc5c(c4)CCNC5=O)c(N)nc3F)cc2)C[C@H](C)N1] | A | C |
| I-145 | 6-(2-amino-6-fluoro-5-(7- (piperidin-4- yloxy)benzo[d][1,3]dioxol- 4-yl)pyridin-3-yl)-3,4- dihydroisoquinolin- 1(2H)-one | 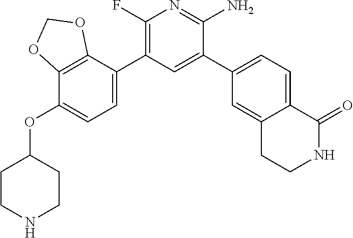 [SMILES: Nc1nc(F)c(-c2ccc(OC3CCNCC3)c3c2OCO3)cc1-c1ccc2c(c1)CCNC2=O] | A | D |
| I-146 | 6-(2-amino-5-(4- (azetidin-3- yloxy)phenyl)-6- fluoropyridin-3-yl)-3,4- dihydroisoquinolin- 1(2H)-one | 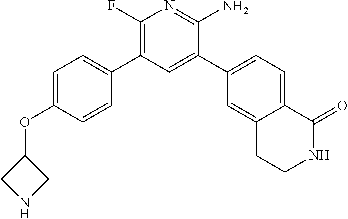 [SMILES: Nc1nc(F)c(-c2ccc(OC3CNC3)cc2)cc1-c1ccc2c(c1)CCNC2=O] | B | D |
| I-147 | 6-(2-amino-5-(4-((2,2- dimethylpiperidin-4- yl)oxy)phenyl)-6- fluoropyridin-3-yl)-3,4- dihydroisoquinolin- 1(2H)-one | 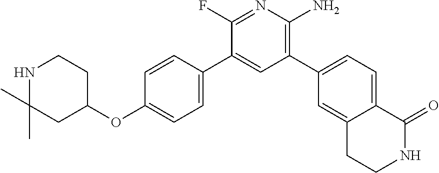 [SMILES: CC1(C)CC(Oc2ccc(-c3cc(-c4ccc5c(c4)CCNC5=O)c(N)nc3F)cc2)CCN1] | A | D |
| I-148 | 6-(2-amino-5-(2,3- difluoro-4-(piperidin-4- ylxoy)phenyl)-6- fluoropyridin-3-yl)-3,4- dihydroisoquinolin- 1(2H)-one | 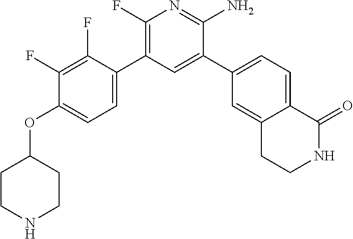 [SMILES: Nc1nc(F)c(-c2ccc(OC3CCNCC3)c(F)c2F)cc1-c1ccc2c(c1)CCNC2=O] | B | D |
| I-149 | 6-(2-amino-5-(2,5- difluoro-4-(piperidin-4- yloxy)phenyl)-6- fluoropyridin-3-yl)-3,4- dihydroisoquinolin- 1(2H)-one | 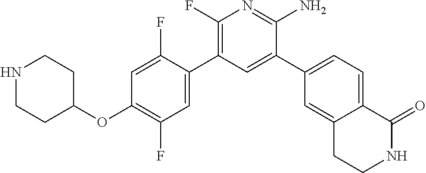 [SMILES: Nc1nc(F)c(-c2cc(F)c(OC3CCNCC3)cc2F)cc1-c1ccc2c(c1)CCNC2=O] | B | D |
| I-150 | 6-(2-amino-6-fluoro-5-(2- methoxy-4-(piperidin-4- yloxy)phenyl)pyridin-3- yl)-3,4- dihydroisoquinolin- 1(2H)-one | 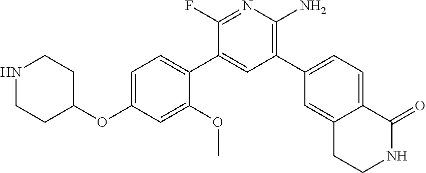 [SMILES: COc1cc(OC2CCNCC2)ccc1-c1cc(-c2ccc3c(c2)CCNC3=O)c(N)nc1F] | B | D |
| I-151 | 6-(2-amino-6-fluoro-5-(3- methoxy-4-(piperidin-4- yloxy)phenyl)pyridin-3- yl)-3,4- dihydroisoquinolin- 1(2H)-one | 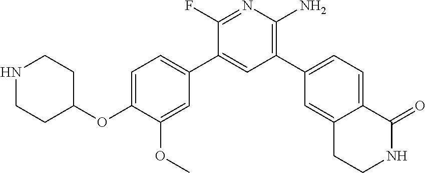 [SMILES: COc1cc(-c2cc(-c3ccc4c(c3)CCNC4=O)c(N)nc2F)ccc1OC1CCNCC1] | B | D |
| I-152 | 6-(2-amino-6-fluoro-5-(4- (1-(oxetan-3-yl)piperidin- 4-yl)phenyl)pyridin-3-yl)- 3,4-dihydroisoquinolin- 1(2H)-one | 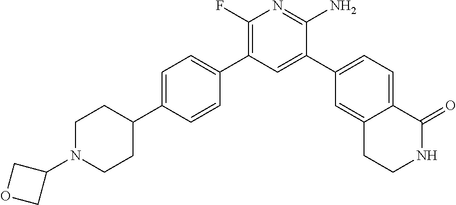 [SMILES: Nc1nc(F)c(-c2ccc(C3CCN(C4COC4)CC3)cc2)cc1-c1ccc2c(c1)CCNC2=O] | A | D |
| I-153 | 6-(2-amino-6-fluoro-5-(4- (1-(oxetan-3-yl)piperidin- 2-yl)phenyl)pyridin-3-yl)- 3,4-dihydroisoquinolin- 1(2H)-one | 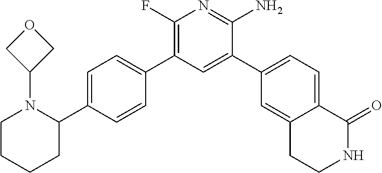 [SMILES: Nc1nc(F)c(-c2ccc(C3CCCCN3C3COC3)cc2)cc1-c1ccc2c(c1)CCNC2=O] | B | D |
| I-154 | 6-(2-amino-6-fluoro-5-(4- (4-(oxetan-3- yl)morpholin-3- yl)phenyl)pyridin-3-yl)- 3,4-dihydroisoquinolin- 1(2H)-one | 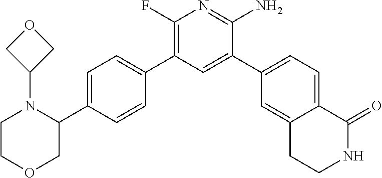 [SMILES: Nc1nc(F)c(-c2ccc(C3COCCN3C3COC3)cc2)cc1-c1ccc2c(c1)CCNC2=O] | B | D |
| I-155 | 6-(2-amino-6-fluoro-5-(4- (1-(oxetan-3-yl)-1,2,3,6- tetrahydropyridin-4- yl)phenyl)pyridin-3-yl)- 3,4-dihydroisoquinolin- 1(2H)-one | 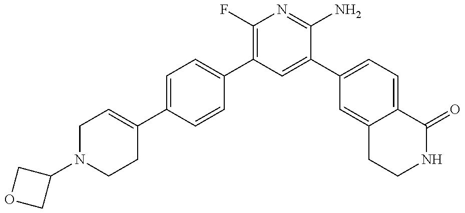 [SMILES: Nc1nc(F)c(-c2ccc(C3=CCN(C4COC4)CC3)cc2)cc1-c1ccc2c(c1)CCNC2=O] | A | D |
| I-156 | 6-(2-amino-6-fluoro-5-(4- ((1R,5S)-3-(oxetan-3-yl)- 3- azabicyclo[3.1.0]hexan- 1-yl)phenyl)pyridin-3-yl)- 3,4-dihydroisoquinolin- 1(2H)-one | 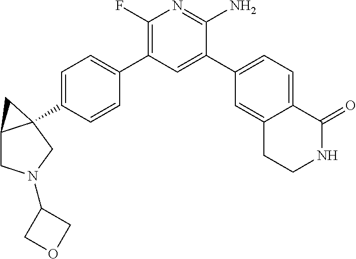 [SMILES: Nc1nc(F)c(-c2ccc([C@@]34C[C@@H]3CN(C3COC3)C4)cc2)cc1-c1ccc2c(c1)CCNC2=O] | A | D |
| I-157 | 6-(2-amino-5-(4-(3- (dimethylamino)pyrrolidin- 1-yl)phenyl)-6- fluoropyridin-3-yl)-3,4- dihydroisoquinolin- 1(2H)-one | 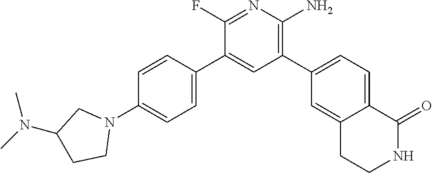 [SMILES: CN(C)C1CCN(c2ccc(-c3cc(-c4ccc5c(c4)CCNC5=O)c(N)nc3F)cc2)C1] | A | D |
| I-158 | 6-(2-amino-5-(4-(3- (ethyl(methyl)amino)pyrro- lidin-1-yl)phenyl)-6- fluoropyridin-3-yl)-3,4- dihydroisoquinolin- 1(2H)-one | 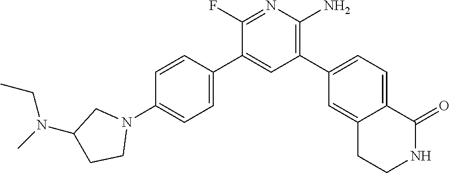 [SMILES: CCN(C)C1CCN(c2ccc(-c3cc(-c4ccc5c(c4)CCNC5=O)c(N)nc3F)cc2)C1] | A | C |
| I-159 | 6-(2-amino-6-fluoro-5-(4- (3-(methyl(2,2,2- trifluoroethyl)amino)pyrro- lidin-1-yl)phenyl)pyridin- 3-yl)-3,4- dihydroisoquinolin- 1(2H)-one | 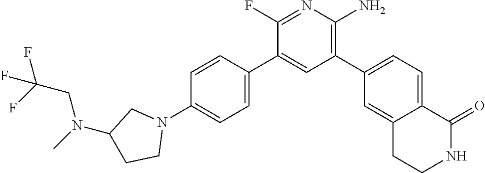 [SMILES: CN(CC(F)(F)F)C1CCN(c2ccc(-c3cc(-c4ccc5c(c4)CCNC5=O)c(N)nc3F)cc2)C1] | A | D |
| I-160 | (R)-6-(2-amino-6-fluoro- 5-(4-(3-((2- methoxyethyl)(methyl)a- mino)pyrrolidin-1- yl)phenyl)pyridin-3-yl)- 3,4-dihydroisoquinolin- 1(2H)-one | 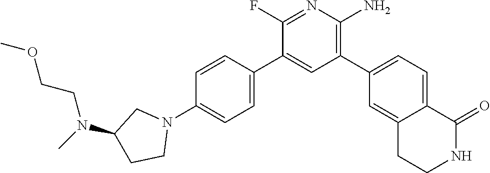 [SMILES: COCCN(C)[C@@H]1CCN(c2ccc(-c3cc(-c4ccc5c(c4)CCNC5=O)c(N)nc3F)cc2)C1] | A | D |
| I-161 | (R)-6-(2-amino-5-(4-(3- ((cyclopropylmethyl)(meth- yl)amino)pyrrolidin-1- yl)phenyl)-6- fluoropyridin-3-yl)-3,4- dihydroisoquinolin- 1(2H)-one | 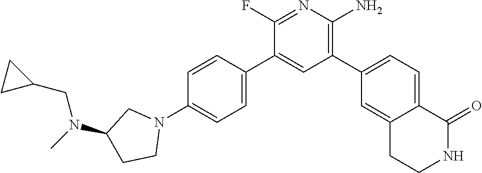 [SMILES: CN(CC1CC1)[C@@H]1CCN(c2ccc(-c3cc(-c4ccc5c(c4)CCNC5=O)c(N)nc3F)cc2)C1] | A | C |
| I-162 | 6-(2-amino-6-fluoro-5-(4- (3- (isobutyl(methyl)amino)pyr- rolidin-1- yl)phenyl)pyridin-3-yl)- 3,4-dihydroisoquinolin- 1(2H)-one | 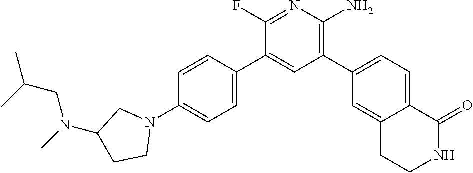 [SMILES: CC(C)CN(C)C1CCN(c2ccc(-c3cc(-c4ccc5c(c4)CCNC5=O)c(N)nc3F)cc2)C1] | A | D |
| I-163 | 6-(2-amino-6-fluoro-5-(4- (4-fluoropiperidin-1- yl)phenyl)pyridin-3-yl)- 3,4-dihydroisoquinolin- 1(2H)-one | 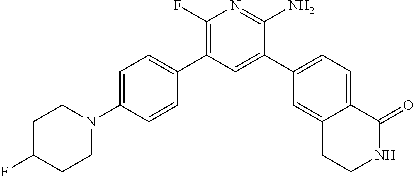 [SMILES: Nc1nc(F)c(-c2ccc(N3CCC(F)CC3)cc2)cc1-c1ccc2c(c1)CCNC2=O] | A | D |
| I-164 | 6-(2-amino-6-fluoro-5-(4- (1-isopropyl-3- methylpyrrolidin-3- yl)phenyl)pyridin-3-yl)- 3,4-dihydroisoquinolin- 1(2H)-one | 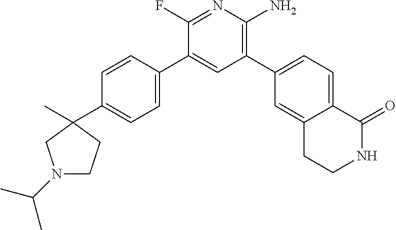 [SMILES: CC(C)N1CCC(C)(c2ccc(-c3cc(-c4ccc5c(c4)CCNC5=O)c(N)nc3F)cc2)C1] | A | D |
| I-165 | 6-(2-amino-6-fluoro-5-(4- ((1S,5R)-3-(2- methoxyethyl)-3- azabicyclo[3.1.0]hexan- 1-yl)phenyl)pyridin-3-yl)- 3,4-dihydroisoquinolin- 1(2H)-one | 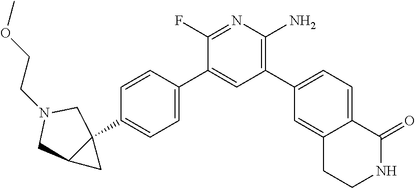 [SMILES: COCCN1C[C@@H]2C[C@]2(c2ccc(-c3cc(-c4ccc5c(c4)CCNC5=O)c(N)nc3F)cc2)C1] | A | D |
| I-166 | 6-(2-amino-6-fluoro-5- (4-(1-(2- methoxyethyl)piperidin- 4-yl)phenyl)pyridin-3-yl)- 3,4-dihydroisoquinolin- 1(2H)-one | 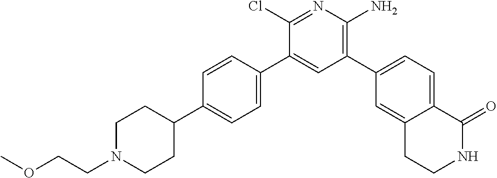 [SMILES: COCCN1CCC(c2ccc(-c3cc(-c4ccc5c(c4)CCNC5=O)c(N)nc3Cl)cc2)CC1] | B | D |
| I-167 | 6-(2-amino-6-fluoro-5-(4- (4-hydroxy-1-(2- methoxyethyl)piperidin- 4-yl)phenyl)pyridin-3-yl)- 3,4-dihydroisoquinolin- 1(2H)-one | 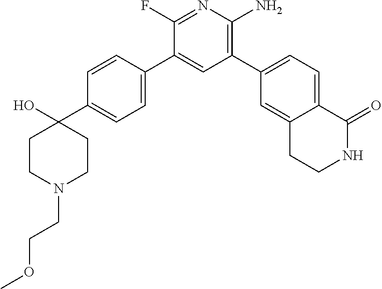 [SMILES: COCCN1CCC(O)(c2ccc(-c3cc(-c4ccc5c(c4)CCNC5=O)c(N)nc3F)cc2)CC1] | A | D |
| I-168 | 6-(2-amino-6-chloro-5- (4-(4-(2,2- difluoroethyl)piperazin-1- yl)phenyl)pyridin-3-yl)- 3,4-dihydroisoquinolin- 1(2H)-one | 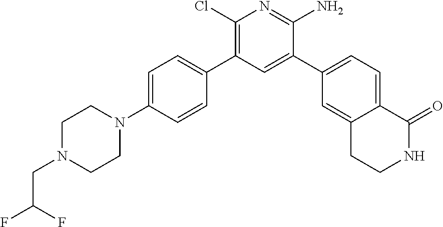 [SMILES: Nc1nc(Cl)c(-c2ccc(N3CCN(CC(F)F)CC3)cc2)cc1-c1ccc2c(c1)CCNC2=O] | B | D |
| I-169 | 6-(2-amino-6-chloro-5- (4-(4-isopropylpiperazin- 1-yl)phenyl)pyridin-3-yl)- 3,4-dihydroisoquinolin- 1(2H)-one | 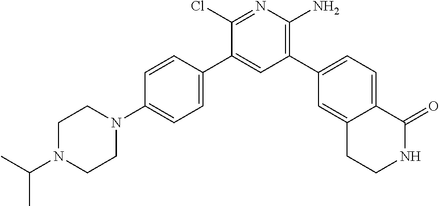 [SMILES: CC(C)N1CCN(c2ccc(-c3cc(-c4ccc5c(c4)CCNC5=O)c(N)nc3Cl)cc2)CC1] | B | D |
| I-170 | 6-(6′-amino-2′-fluoro-5- (4-isopropylpiperazin-1- yl)-[2,3′-bipyridin]-5′-yl)- 7-fluoro-3,4- dihydroisoquinolin- 1(2H)-one | 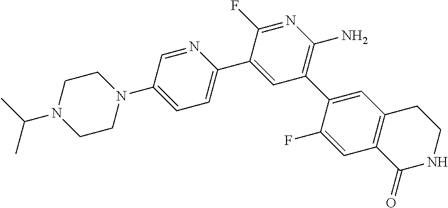 [SMILES: CC(C)N1CCN(c2ccc(-c3cc(-c4cc5c(cc4F)C(=O)NCC5)c(N)nc3F)nc2)CC1] | A | D |
| I-171 | 6-(2-amino-5-(4-(6,6- difluoro-3-isopropyl-3- azabicyclo[3.1.0]hexan- 1-yl)phenyl)-6- fluoropyridin-3-yl)-3,4- dihydroisoquinolin- 1(2H)-one | 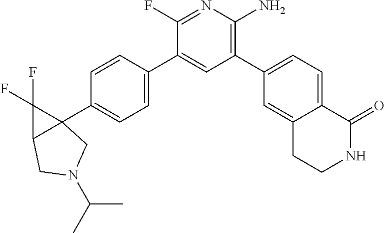 [SMILES: CC(C)N1CC2C(F)(F)C2(c2ccc(-c3cc(-c4ccc5c(c4)CCNC5=O)c(N)nc3F)cc2)C1] | A | D |
| I-172 | 6-(2-amino-6-fluoro-5-(4- (piperazin-1- yl)phenyl)pyridin-3-yl)-7- fluoro-3,4- dihydroisoquinolin- 1(2H)-one 2,2,2- trifluoroacetate | 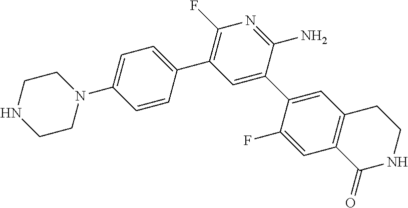 [SMILES: Nc1nc(F)c(-c2ccc(N3CCNCC3)cc2)cc1-c1cc2c(cc1F)C(=O)NCC2] | A | D |
| I-173 | tert-butyl 6-(6-amino-2- fluoro-5-(1-oxo-1,2,3,4- tetrahydroisoquinolin-6- yl)pyridin-3-yl)-4H- spiro[benzo[d][1,3]dioxine- 2,4′-piperidine]-1′- carboxylate | 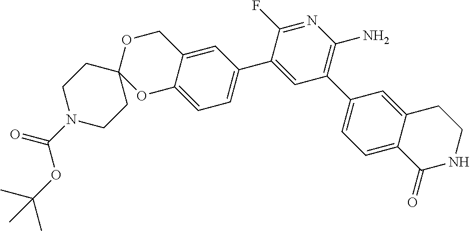 [SMILES: CC(C)(C)OC(=O)N1CCC2(CC1)OCc1cc(-c3cc(-c4ccc5c(c4)CCNC5=O)c(N)nc3F)ccc1O2] | B | D |
| I-174 | tert-butyl 6-(6-amino-2- fluoro-5-(1-oxo-1,2,3,4- tetrahydroisoquinolin-6- yl)pyridin-3-yl)-4- oxospiro[chromane-2,4′- piperidine]-1′-carboxylate | 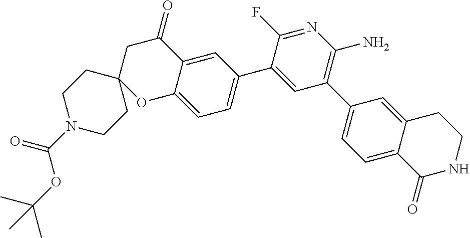 [SMILES: CC(C)(C)OC(=O)N1CCC2(CC1)CC(=O)c1cc(-c3cc(-c4ccc5c(c4)CCNC5=O)c(N)nc3F)ccc1O2] | B | D |
| I-175 | tert-butyl 6-(6-amino-2- fluoro-5-(1-oxo-1,2,3,4- tetrahydroisoquinolin-6- yl)pyridin-3- yl)spiro[chromane-2,4′- piperidine]-1′-carboxylate | 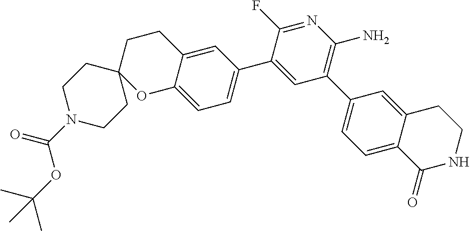 [SMILES: CC(C)(C)OC(=O)N1CCC2(CCc3cc(-c4cc(-c5ccc6c(c5)CCNC6=O)c(N)nc4F)ccc3O2)CC1] | C | D |
| I-176 | tert-butyl 4-(4-(6-amino- 2-fluoro-5-(1-oxo- 1,2,3,4- tetrahydroisoquinolin-6- yl)pyridin-3-yl)-2- cyanophenoxy)piperidine- 1-carboxylate | 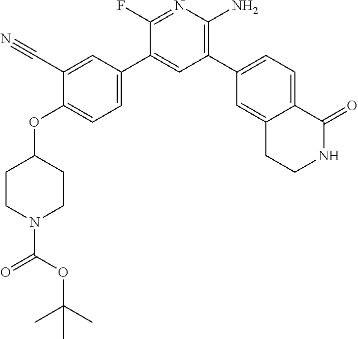 [SMILES: CC(C)(C)OC(=O)N1CCC(Oc2ccc(-c3cc(-c4ccc5c(c4)CCNC5=O)c(N)nc3F)cc2C#N)CC1] | B | D |
| I-177 | tert-butyl 4-(4-(6-amino- 2-fluoro-5-(1-oxo- 1,2,3,4- tetrahydroisoquinolin-6- yl)pyridin-3-yl)-2- (trifluoromethyl)phenyl)pi- perazine-1-carboxylate | 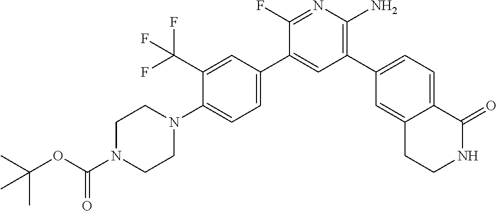 [SMILES: CC(C)(C)OC(=O)N1CCN(c2ccc(-c3cc(-c4ccc5c(c4)CCNC5=O)c(N)nc3F)cc2C(F)(F)F)CC1] | C | D |
| I-178 | 6-(2-amino-5-(4- (((2S,4s,6R)-2,6- dimethylpiperidin-4- yl)oxy)phenyl)-6- fluoropyridin-3-yl)-3,4- dihydroisoquinolin- 1(2H)-one | 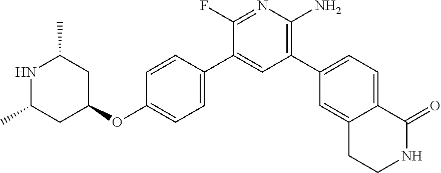 [SMILES: C[C@@H]1C[C@@H](Oc2ccc(-c3cc(-c4ccc5c(c4)CCNC5=O)c(N)nc3F)cc2)C[C@H](C)N1] | A | D |
| I-179 | 6-(2-amino-6-fluoro-5-(3- fluoro-4-(piperidin-4- ylxoy)phenyl)pyridin-3- yl)-3,4- dihydroisoquinolin- 1(2H)-one | 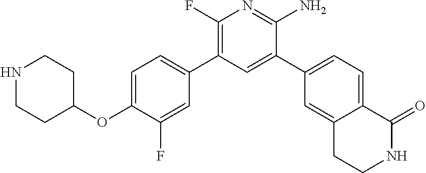 [SMILES: Nc1nc(F)c(-c2ccc(OC3CCNCC3)c(F)c2)cc1-c1ccc2c(c1)CCNC2=O] | A | D |
| I-180 | (R)-6-(2-amino-6-fluoro- 5-(4-(piperidin-3- yloxy)phenyl)pyridin-3- yl)-7-fluoro-3,4- dihydroisoquinolin- 1(2H)-one | 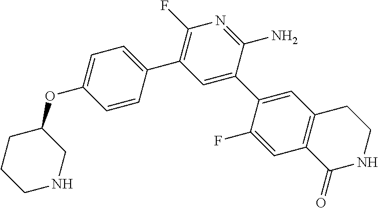 [SMILES: Nc1nc(F)c(-c2ccc(O[C@@H]3CCCNC3)cc2)cc1-c1cc2c(cc1F)C(=O)NCC2] | B | D |
| I-181 | 6-(2-amino-6-fluoro-5- (4H- spiro[benzo[d][1,3]dioxine- 2,4′-piperidin]-6- yl)pyridin-3-yl)-3,4- dihydroisoquinolin- 1(2H)-one | 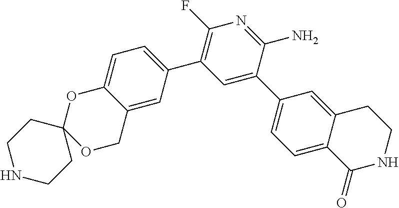 [SMILES: Nc1nc(F)c(-c2ccc3c(c2)COC2(CCNCC2)O3)cc1-c1ccc2c(c1)CCNC2=O] | A | D |
| I-182 | 6-(6-amino-2-fluoro-5-(1- oxo-1,2,3,4- tetrahydroisoquinolin-6- yl)pyridin-3- yl)spiro[chromane-2,4′- piperidin]-4-one 2,2,2- trifluoroacetate | 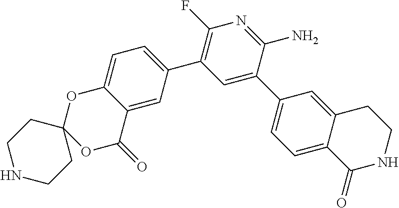 [SMILES: Nc1nc(F)c(-c2ccc3c(c2)C(=O)OC2(CCNCC2)O3)cc1-c1ccc2c(c1)CCNC2=O] | A | D |
| I-183 | 6-(2-amino-6-fluoro-5- (spiro[chromane-2,4′- piperidin]-6-yl)pyridin-3- yl)-3,4- dihydroisoquinolin- 1(2H)-one | 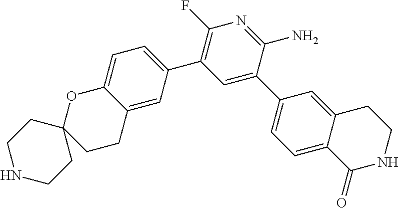 [SMILES: Nc1nc(F)c(-c2ccc3c(c2)CCC2(CCNCC2)O3)cc1-c1ccc2c(c1)CCNC2=O] | A | D |
| I-184 | 5-(6-amino-2-fluoro-5-(1- oxo-1,2,3,4- tetrahydroisoquinolin-6- yl)pyridin-3-yl)-2- (piperidin-4- yloxy)benzonitrile | 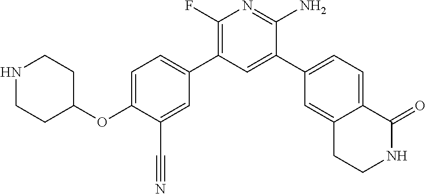 [SMILES: N#Cc1cc(-c2cc(-c3ccc4c(c3)CCNC4=O)c(N)nc2F)ccc1OC1CCNCC1] | B | D |
| I-185 | 6-(2-amino-6-fluoro-5-(4- (piperazin-1-yl)-3- (trifluoromethyl)phenyl)py- ridin-3-yl)-3,4- dihydroisoquinolin- 1(2H)-one | 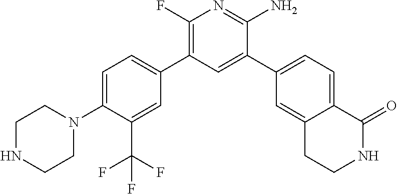 [SMILES: Nc1nc(F)c(-c2ccc(N3CCNCC3)c(C(F)(F)F)c2)cc1-c1ccc2c(c1)CCNC2=O] | A | D |
| I-186 | 6-(2-amino-6-fluoro-5-(4- ((1-(2,2,2- trifluoroethyl)piperidin-4- yl)oxy)phenyl)pyridin-3- yl)-3,4- dihydroisoquinolin- 1(2H)-one | 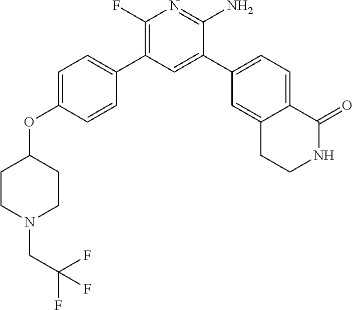 [SMILES: Nc1nc(F)c(-c2ccc(OC3CCN(CC(F)(F)F)CC3)cc2)cc1-c1ccc2c(c1)CCNC2=O] | B | D |
| I-187 | 6-(2-amino-6-fluoro-5-(4- ((1-(3,3,3- trifluoropropyl)piperidin- 4-yl)oxy)phenyl)pyridin- 3-yl)-3,4- dihydroisoquinolin- 1(2H)-one | 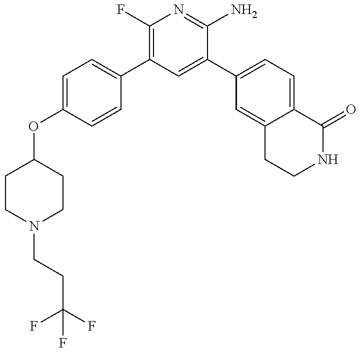 [SMILES: Nc1nc(F)c(-c2ccc(OC3CCN(CCC(F)(F)F)CC3)cc2)cc1-c1ccc2c(c1)CCNC2=O] | A | D |
| I-188 | 6-(2-amino-6-fluoro-5-(4- ((1-(4,4,4- trifluorobutyl)piperidin-4- yl)oxy)phenyl)pyridin-3- yl)-3,4- dihydroisoquinolin- 1(2H)-one | 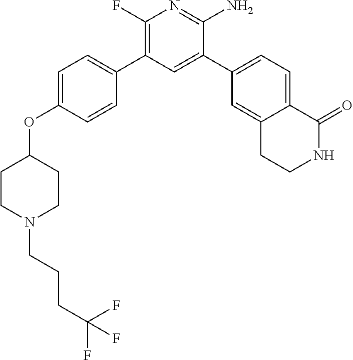 [SMILES: Nc1nc(F)c(-c2ccc(OC3CCN(CCCC(F)(F)F)CC3)cc2)cc1-c1ccc2c(c1)CCNC2=O] | A | D |
| I-189 | 6-(2-amino-6-fluoro-5-(4- ((1-(2- methoxyethyl)piperidin- 4-yl)oxy)phenyl)pyridin- 3-yl)-3,4- dihydroisoquinolin- 1(2H)-one | 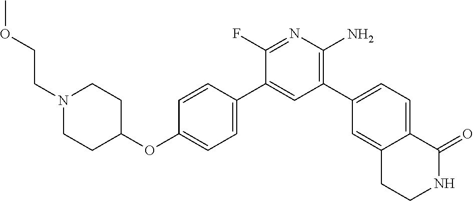 [SMILES: COCCN1CCC(Oc2ccc(-c3cc(-c4ccc5c(c4)CCNC5=O)c(N)nc3F)cc2)CC1] | A | D |
| I-190 | 6-(2-amino-6-fluoro-5-(4- (piepridin-4-yloxy)-3- (trifluoromethyl)phenyl)py- ridin-3-yl)-3,4- dihydroisoquinolin- 1(2H)-one | 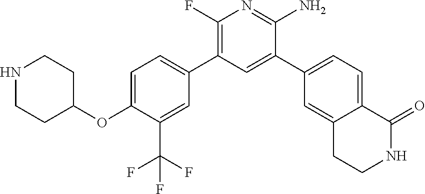 [SMILES: Nc1nc(F)c(-c2ccc(OC3CCNCC3)c(C(F)(F)F)c2)cc1-c1ccc2c(c1)CCNC2=O] | B | |
| I-191 | 6-(5-(1′-acetyl-4H- spiro[benzo[d][1,3]dioxine- 2,4′-piperidin]-6-yl)-2- amino-6-fluoropyridin-3- yl)-3,4- dihydroisoquinolin- 1(2H)-one | 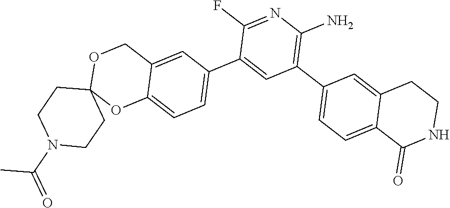 [SMILES: CC(=O)N1CCC2(CC1)OCc1cc(-c3cc(-c4ccc5c(c4)CCNC5=O)c(N)nc3F)ccc1O2] | A | D |
| I-192 | 1′-acetyl-6-(6-amino-2- fluoro-5-(1-oxo-1,2,3,4- tetrahydroisoquinolin-6- yl)pyridin-3- yl)spiro[chromane-2,4′- piepridin]-4-one | 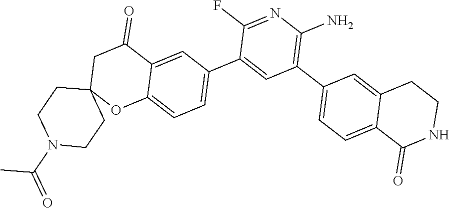 [SMILES: CC(=O)N1CCC2(CC1)CC(=O)c1cc(-c3cc(-c4ccc5c(c4)CCNC5=O)c(N)nc3F)ccc1O2] | A | D |
| I-193 | 6-(5-(1′- acetylspiro[chromane- 2,4′-piperidin]-6-yl)-2- amino-6-fluoropyridin-3- yl)-3,4- dihydroisoquinolin- 1(2H)-one | 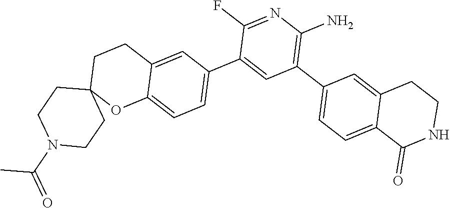 [SMILES: CC(=O)N1CCC2(CCc3cc(-c4cc(-c5ccc6c(c5)CCNC6=O)c(N)nc4F)ccc3O2)CC1] | A | D |
| I-194 | 2-((1-acetylpiperidin-4- yl)oxy)-5-(6-amino-2- fluoro-5-(1-oxo-1,2,3,4- tetrahydroisoquinolin-6- yl)pyridine-3- yl)benzonitrile | 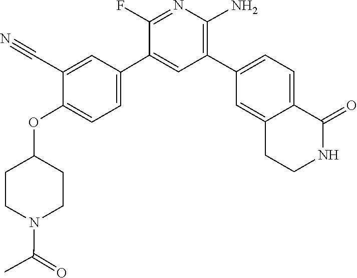 [SMILES: CC(=O)N1CCC(Oc2ccc(-c3cc(-c4ccc5c(c4)CCNC5=O)c(N)nc3F)cc2C#N)CC1] | A | D |
| I-195 | 6-(2-amino-6-fluoro-5- (1′-(2,2,2-trifluoroethyl)- 4H- spiro[benzo[d][1,3]dioxine- 2,4′-piperidin]-6- yl)pyridin-3-yl)-3,4- dihydroisoquinolin- 1(2H)-one | 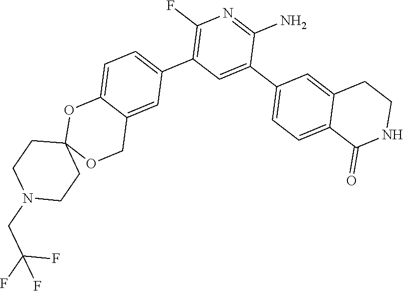 [SMILES: Nc1nc(F)c(-c2ccc3c(c2)COC2(CCN(CC(F)(F)F)CC2)O3)cc1-c1ccc2c(c1)CCNC2=O] | B | D |
| I-196 | 6-(6-amino-2-fluoro-5-(1- oxo-1,2,3,4- tetrahydroisoquinolin-6- yl)pyridin-3-yl)-1′-(2,2,2- trifluoroethyl)spiro[chromane- 2,4′-piperidin]-4-one | 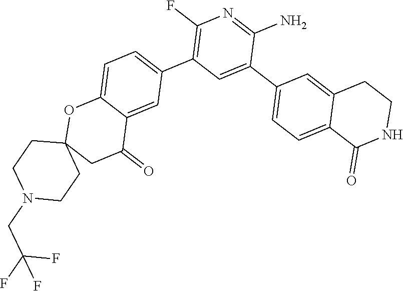 [SMILES: Nc1nc(F)c(-c2ccc3c(c2)C(=O)CC2(CCN(CC(F)(F)F)CC2)O3)cc1-c1ccc2c(c1)CCNC2=O] | A | D |
| I-197 | 6-(2-amino-6-fluoro-5- (1′-(2,2,2- trifluoroethyl)spiro[chromane- 2,4′-piperidin]-6- yl)pyridin-3-yl)-3,4- dihydroisoquinolin- 1(2H)-one | 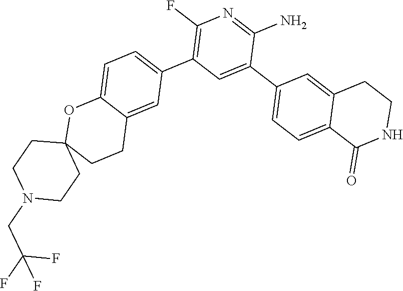 [SMILES: Nc1nc(F)c(-c2ccc3c(c2)CCC2(CCN(CC(F)(F)F)CC2)O3)cc1-c1ccc2c(c1)CCNC2=O] | B | D |
| I-198 | 5-(6-amino-2-fluoro-5-(1- oxo-1,2,3,4- tetrahydroisoquinolin-6- yl)pyridin-3-yl)-2-((1- (2,2,2- trifluoroethyl)piperidin-4- yl)oxy)benzonitrile | 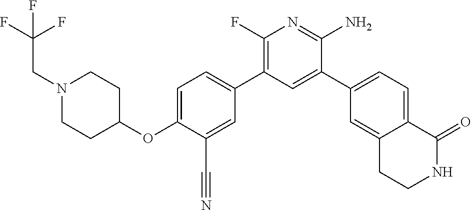 [SMILES: N#Cc1cc(-c2cc(-c3ccc4c(c3)CCNC4=O)c(N)nc2F)ccc1OC1CCN(CC(F)(F)F)CC1] | B | D |
| I-199 | 6-(5-(4-((1- acetylpiperidin-4-yl)oxy)- 3- (trifluoromethyl)phenyl)- 2-amino-6-fluoropyridin- 3-yl)-3,4- dihydroisoquinolin- 1(2H)-one | 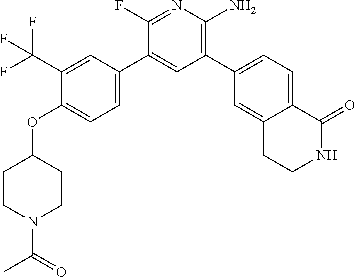 [SMILES: CC(=O)N1CCC(Oc2ccc(-c3cc(-c4ccc5c(c4)CCNC5=O)c(N)nc3F)cc2C(F)(F)F)CC1] | B | D |
| I-200 | 6-(5-(4-(4- acetylpiperazin-1-yl)-3- (trifluoromethyl)phenyl)- 2-amino-6-fluoropyridin- 3-yl)-3,4- dihydroisoquinolin- 1(2H)-one | 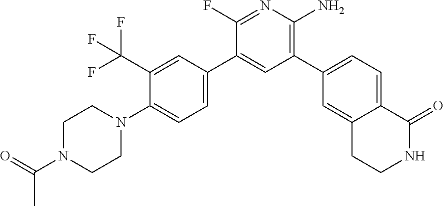 [SMILES: CC(=O)N1CCN(c2ccc(-c3cc(-c4ccc5c(c4)CCNC5=O)c(N)nc3F)cc2C(F)(F)F)CC1] | A | D |
| I-201 | 6-(2-amino-6-fluoro-5-(4- ((1-(2,2,2- trifluoroethyl)piperidin-4- yl)oxy)-3- (trifluoromethyl)phenyl)py- ridin-3-yl)-3,4- dihydroisoquinolin- 1(2H)-one | 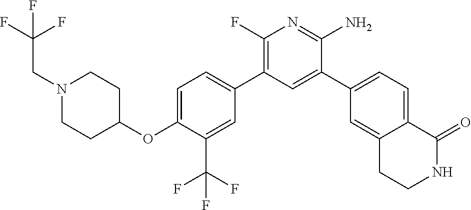 [SMILES: Nc1nc(F)c(-c2ccc(OC3CCN(CC(F)(F)F)CC3)c(C(F)(F)F)c2)cc1-c1ccc2c(c1)CCNC2=O] | C | D |
| I-202 | 6-(2-amino-6-fluoro-5-(4- (4-(2,2,2- trifluoroethyl)piperazin-1- yl)-3- (trifluoromethyl)phenyl)py- ridin-3-yl)-3,4- dihydroisoquinolin- 1(2H)-one | 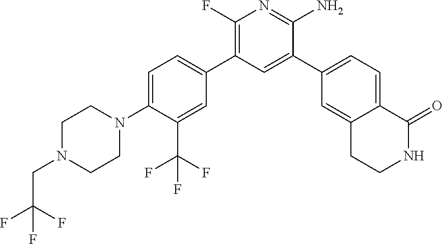 [SMILES: Nc1nc(F)c(-c2ccc(N3CCN(CC(F)(F)F)CC3)c(C(F)(F)F)c2)cc1-c1ccc2c(c1)CCNC2=O] | B | D |
| I-203 | 6-(2-amino-5-(4-((1- (cyclopropanecarbonyl)pi- peridin-4-yl)oxy)phenyl)- 6-fluoropyridin-3-yl)-3,4- dihydroisoquinolin- 1(2H)-one | 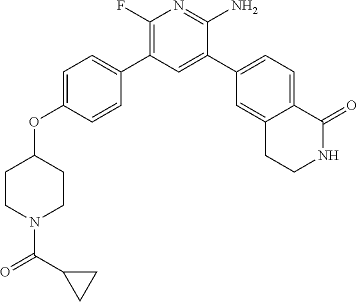 [SMILES: Nc1nc(F)c(-c2ccc(OC3CCN(C(=O)C4CC4)CC3)cc2)cc1-c1ccc2c(c1)CCNC2=O] | A | D |
| I-204 | 6-(2-amino-5-(3,4- dihydro-2H- benzo[b][1,4]dioxepin-7- yl)-6-fluoropyridin-3-yl)- 3,4-dihydroisoquinolin- 1(2H)-one | 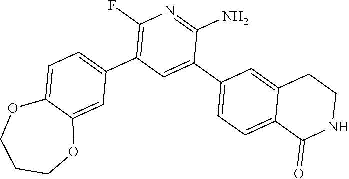 [SMILES: Nc1nc(F)c(-c2ccc3c(c2)OCCCO3)cc1-c1ccc2c(c1)CCNC2=O] | A | D |
| I-205 | 6-(2-amino-5-(3,4- dihydro-2H- benzo[b][1,4]dioxepin-7- yl)-6-fluoropyridin-3-yl)- 7-fluoro-3,4- dihydroisoquinolin- 1(2H)-one | 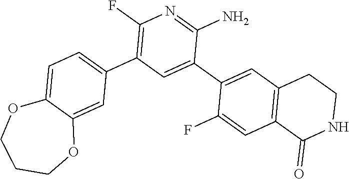 [SMILES: Nc1nc(F)c(-c2ccc3c(c2)OCCCO3)cc1-c1cc2c(cc1F)C(=O)NCC2] | A | D |
| I-206 | 6-(2-amino-6-fluoro-5-(4- ((4-methyl-1,4-diazepan- 1- yl)sulfonyl)phenyl)pyridin- 3-yl)-3,4- dihydroisoquinolin- 1(2H)-one | 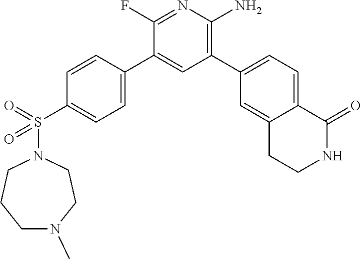 [SMILES: CN1CCCN(S(=O)(=O)c2ccc(-c3cc(-c4ccc5c(c4)CCNC5=O)c(N)nc3F)cc2)CC1] | A | D |
| I-207 | 6-(2-amino-6-fluoro-5-(4- ((4-methylpiperazin-1- yl)sulfonyl)phenyl)pyridin- 3-yl)-3,4- dihydroisoquinolin- 1(2H)-one | 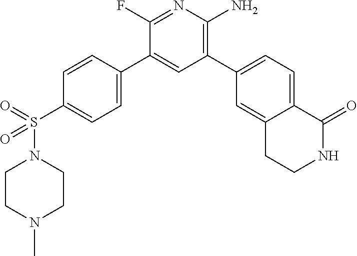 [SMILES: CN1CCN(S(=O)(=O)c2ccc(-c3cc(-c4ccc5c(c4)CCNC5=O)c(N)nc3F)cc2)CC1] | A | D |
| I-208 | 6-(2-amino-6-fluoro-5-(4- (4-methoxy-1-(2- methoxyethyl)piperidin- 4-yl)phenyl)pyridin-3-yl)- 3,4-dihydroisoquinolin- 1(2H)-one | 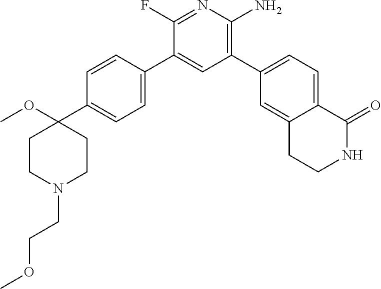 [SMILES: COCCN1CCC(OC)(c2ccc(-c3cc(-c4ccc5c(c4)CCNC5=O)c(N)nc3F)cc2)CC1] | A | D |
| I-209 | 6-(2-amino-6-chloro-5- (4-(4-isopropylpiperazin- 1-yl)phenyl)pyriidn-3-yl)- 7-fluoro-3,4- dihydroisoquinolin- 1(2H)-one | 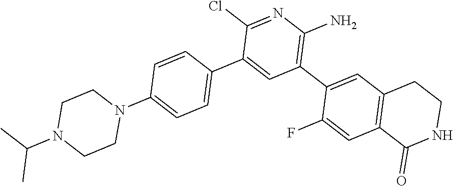 [SMILES: CC(C)N1CCN(c2ccc(-c3cc(-c4cc5c(cc4F)C(=O)NCC5)c(N)nc3Cl)cc2)CC1] | A | D |
| I-210 | 6-(2-amino-5-(4-(4- (cyclopropylmethyl)piper- azin-1-yl)phenyl)-6- fluoropyridin-3-yl)-3,4- dihydroisoquinolin- 1(2H)-one | 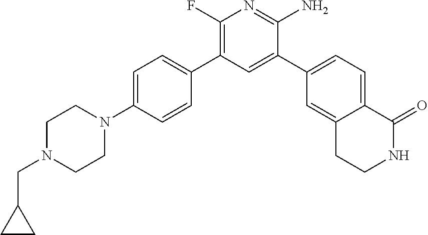 [SMILES: Nc1nc(F)c(-c2ccc(N3CCN(CC4CC4)CC3)cc2)cc1-c1ccc2c(c1)CCNC2=O] | A | C |
| I-211 | 6-(2-amino-5-(4-(4- ethylpiperazin-1- yl)phenyl)-6- fluoropyridin-3-yl)-7- fluoro-3,4- dihydroisoquinolin- 1(2H)-one | 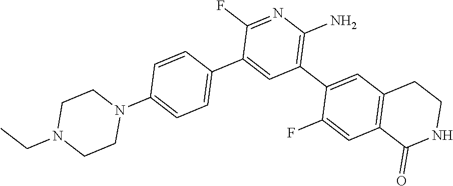 [SMILES: CCN1CCN(c2ccc(-c3cc(-c4cc5c(cc4F)C(=O)NCC5)c(N)nc3F)cc2)CC1] | A | D |
| I-212 | 6-(2-amino-6-fluoro-5-(4- (4-(2- methoxyethyl)piperazin- 1-yl)phenyl)pyridin-3-yl)- 7-fluoro-3,4- dihydroisoquinolin- 1(2H)-one | 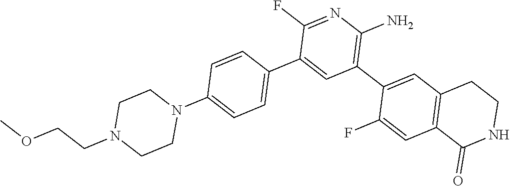 [SMILES: COCCN1CCN(c2ccc(-c3cc(-c4cc5c(cc4F)C(=O)NCC5)c(N)nc3F)cc2)CC1] | A | D |
| I-213 | 6-(2-amino-5-(4-(4- (cyclopropylmethyl)piper- azin-1-yl)phenyl)-6- fluoropyridin-3-yl)-7- fluoro-3,4- dihydroisoquinolin- 1(2H)-one | 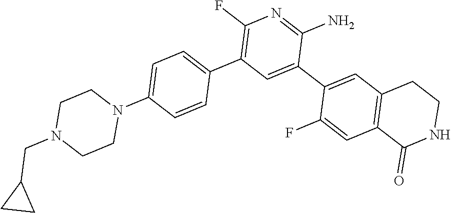 [SMILES: Nc1nc(F)c(-c2ccc(N3CCN(CC4CC4)CC3)cc2)cc1-c1cc2c(cc1F)C(=O)NCC2] | A | D |
| I-214 | 6-(2-amino-6-fluoro-5- (1′-(2-methoxyethyl)-4H- spiro[benzo[d][1,3]dioxine- 2,4′-piperidin]-6- yl)pyridin-3-yl)-3,4- dihydroisoquinolin- 1(2H)-one | 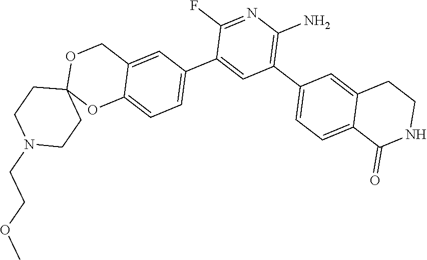 [SMILES: COCCN1CCC2(CC1)OCc1cc(-c3cc(-c4ccc5c(c4)CCNC5=O)c(N)nc3F)ccc1O2] | A | D |
| I-215 | 6-(6-amino-2-fluoro-5-(1- oxo-1,2,3,4- tetrahydroisoquinolin-6- yl)pyridin-3-yl)-1′-(2- methoxyethyl)spiro[chro- mane-2,4′-piperidin]-4- one | 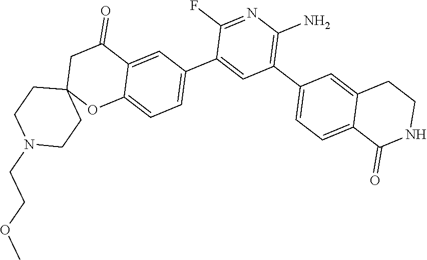 [SMILES: COCCN1CCC2(CC1)CC(=O)c1cc(-c3cc(-c4ccc5c(c4)CCNC5=O)c(N)nc3F)ccc1O2] | A | D |
| I-216 | 6-(2-amio-6-fluoro-5- (1′-(2- methoxyethyl)spiro[chro- mane-2,4′-piperidin]-6- yl)pyridin-3-yl)-3,4- dihydroisoquinolin- 1(2H)-one | 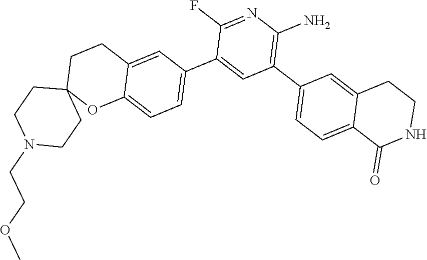 [SMILES: COCCN1CCC2(CCc3cc(-c4cc(-c5ccc6c(c5)CCNC6=O)c(N)nc4F)ccc3O2)CC1] | A | D |
| I-217 | 5-(6-amino-2-fluoro-5-(1- oxo-1,2,3,4- tetrahydroisoquinolin-6- yl)pyridin-3-yl)-2-((1-(2- methoxyethyl)piperidin- 4-yl)oxy)benzonitrile | 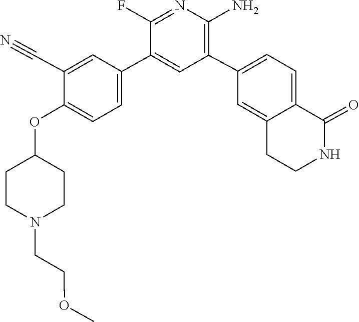 [SMILES: COCCN1CCC(Oc2ccc(-c3cc(-c4ccc5c(c4)CCNC5=O)c(N)nc3F)cc2C#N)CC1] | A | D |
| I-218 | 6-(2-amino-6-fluoro-5- (1′-isopropyl-4H- spiro[benzo[d][1,3]dioxine- 2,4′-piperidin]-6- yl)pyridin-3-yl)-3,4- dihydroisoquinolin- 1(2H)-one | 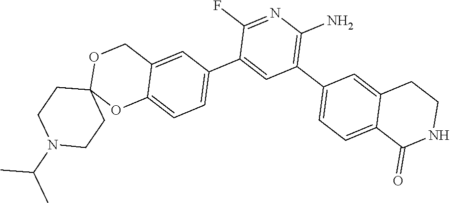 [SMILES: CC(C)N1CCC2(CC1)OCc1cc(-c3cc(-c4ccc5c(c4)CCNC5=O)c(N)nc3F)ccc1O2] | A | D |
| I-219 | 6-(2-amino-6-fluoro-5-(4- (piperazin-1-yl)-3- (trifluoromethyl)phenyl)py- ridin-3-yl)-7-fluoro-3,4- dihydroisoquinolin- 1(2H)-one | 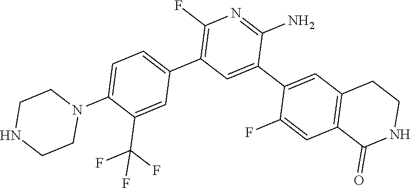 [SMILES: Nc1nc(F)c(-c2ccc(N3CCNCC3)c(C(F)(F)F)c2)cc1-c1cc2c(cc1F)C(=O)NCC2] | A | D |
| I-220 | 6-(2-amino-6-fluoro-5-(4- ((1R,5S)-3-(2- methoxyacetyl)-3- azabicyclo[3.1.0]hexan- 1-yl)phenyl)pyridin-3-yl)- 3,4-dihydroisoquinolin- 1(2H)-one | 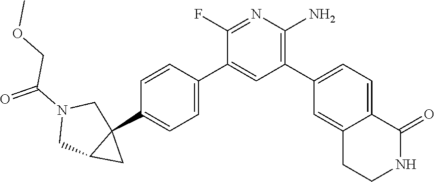 [SMILES: COCC(=O)N1C[C@H]2C[C@@]2(c2ccc(-c3cc(-c4ccc5c(c4)CCNC5=O)c(N)nc3F)cc2)C1] | A | D |
| I-221 | 6-(6′-(4-acetylpiperazin- 1-yl)-6-amino-2-fluoro- [3,3′-bipyridin]-5-yl)-7- fluoro-3,4- dihydroisoquinolin- 1(2H)-one 2,2,2- trifluoroacetate | 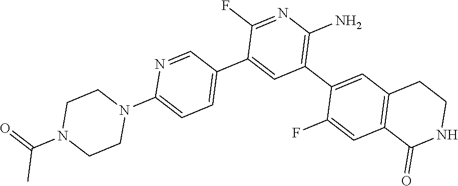 [SMILES: CC(=O)N1CCN(c2ccc(-c3cc(-c4cc5c(cc4F)C(=O)NCC5)c(N)nc3F)cn2)CC1] | B | D |
| I-222 | 6-(6-amino-2-fluoro-5-(1- oxo-1,2,3,4- tetrahydroisoquinolin-6- yl)pyridin-3-yl)-1′- isopropylspiro[chromane- 2,4′-piperidin]-4-one | 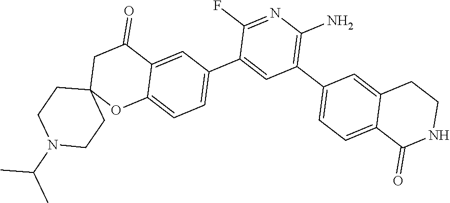 [SMILES: CC(C)N1CCC2(CC1)CC(=O)c1cc(-c3cc(-c4ccc5c(c4)CCNC5=O)c(N)nc3F)ccc1O2] | A | D |
| I-223 | 6-(2-amino-6-fluoro-5- (1′- isopropylspiro[chromane- 2,4′-piperidin]-6- yl)pyridin-3-yl)-3,4- dihydroisoquinolin- 1(2H)-one | 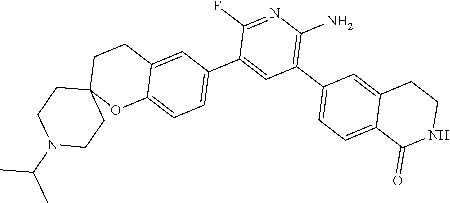 [SMILES: CC(C)N1CCC2(CCc3cc(-c4cc(-c5ccc6c(c5)CCNC6=O)c(N)nc4F)ccc3O2)CC1] | A | D |
| I-224 | 5-(6-amino-2-fluoro-5-(1- oxo-1,2,3,4- tetrahydroisoquinolin-6- yl)pyridin-3-yl)-2-((1- isopropylpiperidin-4- yl)oxy)benzonitrile | 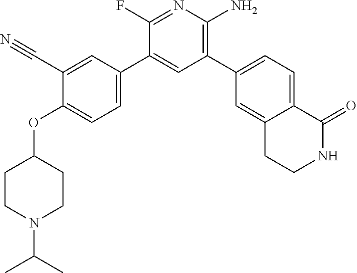 [SMILES: CC(C)N1CCC(Oc2ccc(-c3cc(-c4ccc5c(c4)CCNC5=O)c(N)nc3F)cc2C#N)CC1] | A | D |
| I-225 | 6-(2-amino-5-(4- ((1R,5S)-3- (dimethylglycyl)-3- azabicyclo[3.1.0]hexan- 1-yl)phenyl)-6- fluoropyridin-3-yl)-3,4- dihydroisoquinolin- 1(2H)-one | 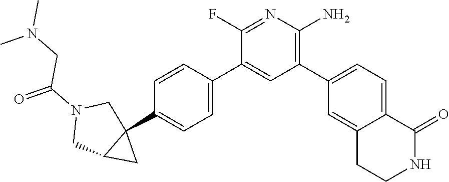 [SMILES: CN(C)CC(=O)N1C[C@H]2C[C@@]2(c2ccc(-c3cc(-c4ccc5c(c4)CCNC5=O)c(N)nc3F)cc2)C1] | A | D |
| I-226 | 6-(6-amino-2-fluoro-6′- (4-isopropyylpiperazin-1- yl)-[3,3′-bipyridin]-5-yl)-7- fluoro-3,4- dihydroisoquinolin- 1(2H)-one | 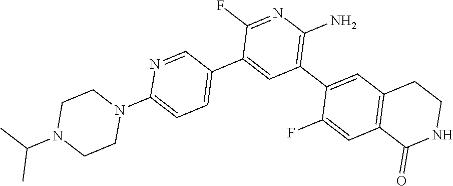 [SMILES: CC(C)N1CCN(c2ccc(-c3cc(-c4cc5c(cc4F)C(=O)NCC5)c(N)nc3F)cn2)CC1] | A | D |
| I-227 | 6-(2-amino-5-(3-amino- 8-chloro-7- fluoroisoquinolin-6-yl)-6- fluoropyridin-3-yl)-3,4- dihydroisoquinolin- 1(2H)-one | 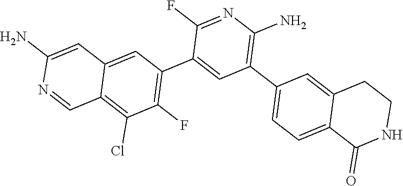 [SMILES: Nc1cc2cc(-c3cc(-c4ccc5c(c4)CCNC5=O)c(N)nc3F)c(F)c(Cl)c2cn1] | B | D |
| I-228 | 2-(6-amino-2-fluoro-5-(1- oxo-1,2,3,4- tetrahydroisoquinolin-6- yl)pyridin-3-yl)-6- isopropyl-5,6-dihydro- 4H-pyrazolo[1,5- d][1,4]diazepin-7(8H)- one | 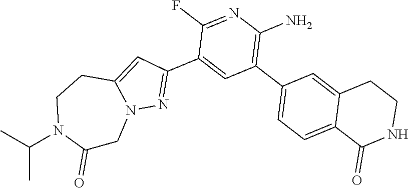 [SMILES: CC(C)N1CCc2cc(-c3cc(-c4ccc5c(c4)CCNC5=O)c(N)nc3F)nn2CC1=O] | B | D |
| I-229 | 2-(6-amino-2-fluoro-5-(1- oxo-1,2,3,4- tetrahydroisoquinolin-6- yl)pyridin-3-yl)-5,6- dihydro-4H-pyrazolo[1,5- d][1,4]diazepin-7(8H)- one | 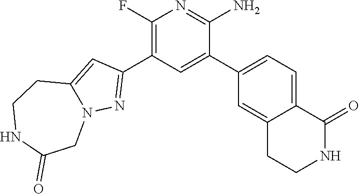 [SMILES: Nc1nc(F)c(-c2cc3n(n2)CC(=O)NCC3)cc1-c1ccc2c(c1)CCNC2=O] | B | D |
| I-230 | 6-(2-amino-6-chloro-5- (4-(1-methyl-1,2,3,6- tetrahydropyridin-4- yl)phenyl)pyridin-3-yl)- 3,4-dihydroisoquinolin- 1(2H)-one | 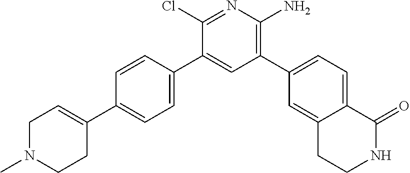 [SMILES: CN1CC=C(c2ccc(-c3cc(-c4ccc5c(c4)CCNC5=O)c(N)nc3Cl)cc2)CC1] | B | D |
| I-231 | 6-(2-amino-6-chloro-5- (4-(1-methylpiperidin-4- yl)phenyl)pyridin-3-yl)- 3,4-dihydroisoquinolin- 1(2H)-one | 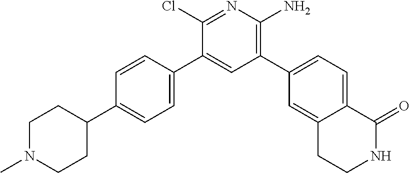 [SMILES: CN1CCC(c2ccc(-c3cc(-c4ccc5c(c4)CCNC5=O)c(N)nc3Cl)cc2)CC1] | B | D |
| I-232 | 6-(2-amino-6-chloro-5- (4-(1-methyl-1,2,3,6- tetrahydropyridin-4- yl)phenyl)pyridin-3-yl)-7- fluoro-3,4- dihydroisoquinolin- 1(2H)-one | 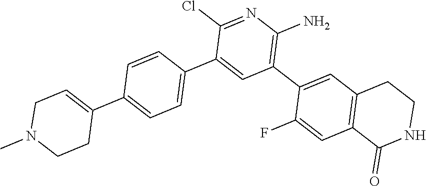 [SMILES: CN1CC=C(c2ccc(-c3cc(-c4cc5c(cc4F)C(=O)NCC5)c(N)nc3Cl)cc2)CC1] | B | D |
| I-233 | 6-(2-amino-6-chloro-5- (4-(1-methylpiperidin-4- yl)phenyl)pyridin-3-yl)-7- fluoro-3,4- dihydroisoquinolin- 1(2H)-one | 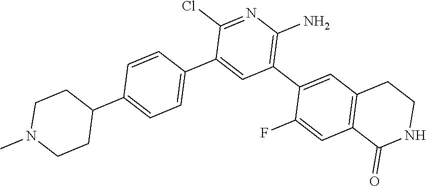 [SMILES: CN1CCC(c2ccc(-c3cc(-c4cc5c(cc4F)C(=O)NCC5)c(N)nc3Cl)cc2)CC1] | B | D |
| I-234 | 6-(5′6-diamino-2-fluoro- 4′-methyl-[3,3′-bipyridin]- 5-yl)-3,4- dihydroisoquinolin- 1(2H)-one 2,2,2- trifluoroacetate | 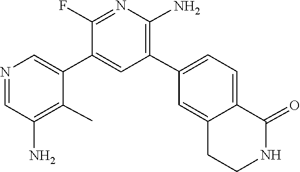 [SMILES: Cc1c(N)cncc1-c1cc(-c2ccc3c(c2)CCNC3=O)c(N)nc1F] | D | D |
| I-235 | 6-(2-amino-5-(4-(1- ethylpyrrolidin-3- yl)phenyl)-6- fluoropyridin-3-yl)-3,4- dihydroisoquinolin- 1(2H)-one | 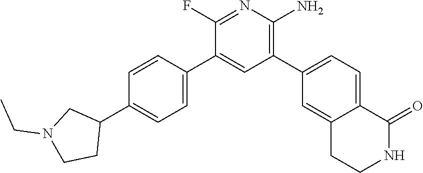 [SMILES: CCN1CCC(c2ccc(-c3cc(-c4ccc5c(c4)CCNC5=O)c(N)nc3F)cc2)C1] | A | D |
| I-236 | 6-(2-amino-5-(4-(4-(2,2- difluoroethyl)piperazin-1- yl)phenyl)-6- fluoropyridin-3-yl)-7- fluoro-3,4- dihydroisoquinolin- 1(2H)-one | 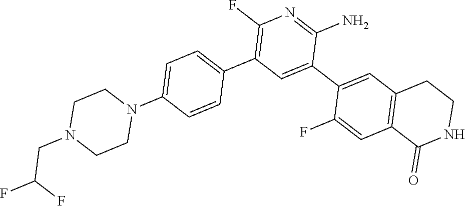 [SMILES: Nc1nc(F)c(-c2ccc(N3CCN(CC(F)F)CC3)cc2)cc1-c1cc2c(cc1F)C(=O)NCC2] | A | D |
| I-237 | 6-(2-amino-6-fluoro-5-(4- (4-(2,2,2- trifluoroethyl)piperazin-1- yl)phenyl)pyridin-3-yl)-7- fluoro-3,4- dihydroisoquinolin- 1(2H)-one | 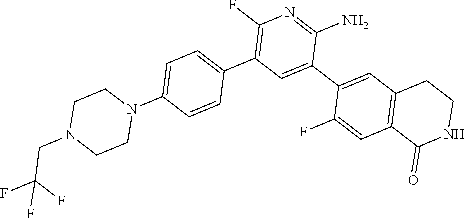 [SMILES: Nc1nc(F)c(-c2ccc(N3CCN(CC(F)(F)F)CC3)cc2)cc1-c1cc2c(cc1F)C(=O)NCC2] | A | D |
| I-238 | 6-(2-amino-6-fluoro-5-(4- (piperidin-4- yl)phenyl)pyridin-3-yl)-7- fluoro-3,4- dihydroisoquinolin- 1(2H)-one 2,2,2- trifluoroacetate | 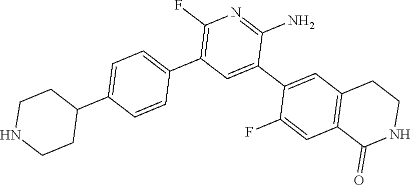 [SMILES: Nc1nc(F)c(-c2ccc(C3CCNCC3)cc2)cc1-c1cc2c(cc1F)C(=O)NCC2] | A | D |
| I-239 | 6-(2-amino-6-fluoro-5-(4- (4-fluoro-1- isopropylpiperidin-4- yl)phenyl)pyridin-3-yl)- 3,4-dihydroisoquinolin- 1(2H)-one | 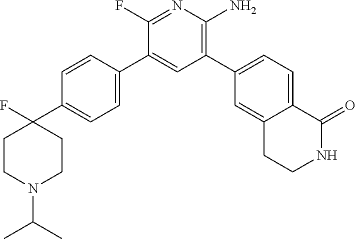 [SMILES: CC(C)N1CCC(F)(c2ccc(-c3cc(-c4ccc5c(c4)CCNC5=O)c(N)nc3F)cc2)CC1] | A | D |
| I-240 | 6-(2-amino-6-fluoro-5-(4- (4-(3,3,3- trifluoropropyl)piperazin- 1-yl)phenyl)pyridin-3-yl)- 3,4-dihydroisoquinolin- 1(2H)-one | 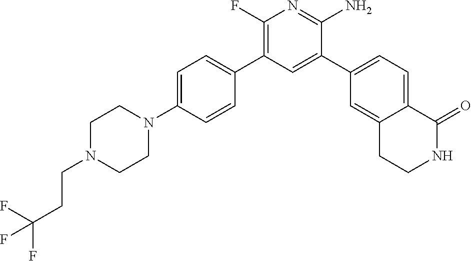 [SMILES: Nc1nc(F)c(-c2ccc(N3CCN(CCC(F)(F)F)CC3)cc2)cc1-c1ccc2c(c1)CCNC2=O] | A | D |
| I-241 | 6-(2-amino-6-fluoro-5-(4- (1-(2- methoxyethyl)piperidin- 4-yl)phenyl)pyridin-3-yl)- 7-fluoro-3,4- dihydroisoquinolin- 1(2H)-one | 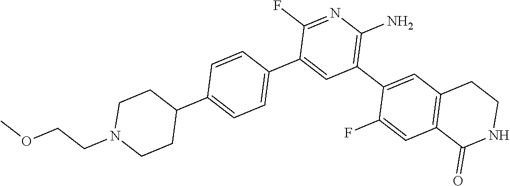 [SMILES: COCCN1CCC(c2ccc(-c3cc(-c4cc5c(cc4F)C(=O)NCC5)c(N)nc3F)cc2)CC1] | A | D |
| I-242 | 6-(2-amino-5-(4-(1- (cyclopropylmethyl)piperi- din-4-yl)phenyl)-6- fluoropyridin-3-yl)-7- fluoro-3,4- dihydroisoquinolin- 1(2H)-one | 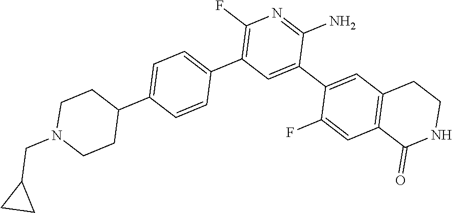 [SMILES: Nc1nc(F)c(-c2ccc(C3CCN(CC4CC4)CC3)cc2)cc1-c1cc2c(cc1F)C(=O)NCC2] | A | D |
| I-243 | 6-(2-amino-6-fluoro-5-(4- (4-(3,3,3- trifluoropropyl)piperazin- 1-yl)phenyl)pyridin-3-yl)- 7-fluoro-3,4- dihydroisoquinolin- 1(2H)-one | 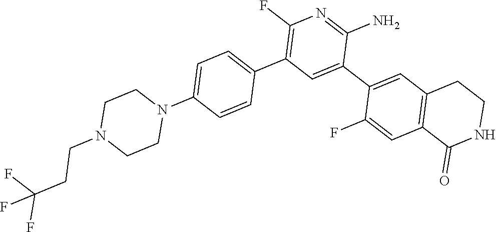 [SMILES: Nc1nc(F)c(-c2ccc(N3CCN(CCC(F)(F)F)CC3)cc2)cc1-c1cc2c(cc1F)C(=O)NCC2] | A | D |
| I-244 | 6-(2-amino-6-fluoro-5-(4- (1-isopropylpiperidin-4- yl)phenyl)pyridin-3-yl)-7- fluoro-3,4- dihydroisoquinolin- 1(2H)-one | 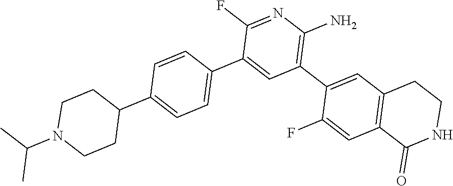 [SMILES: CC(C)N1CCC(c2ccc(-c3cc(-c4cc5c(cc4F)C(=O)NCC5)c(N)nc3F)cc2)CC1] | A | D |
| I-245 | 6-(2-amino-5-(4-(1- ethylpiperidin-4- yl)phenyl)-6- fluoropyridin-3-yl)-7- fluoro-3,4- dihydroisoquinolin- 1(2H)-one | 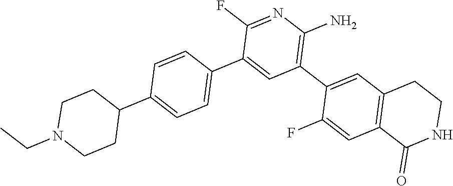 [SMILES: CCN1CCC(c2ccc(-c3cc(-c4cc5c(cc4F)C(=O)NCC5)c(N)nc3F)cc2)CC1] | A | D |
| I-246 | 6-(2-amino-6-fluoro-5-(4- (4-methylpiperazin-1- yl)phenyl)pyridin-3-yl)-7- fluoro-3,4- dihydroisoquinolin- 1(2H)-one | 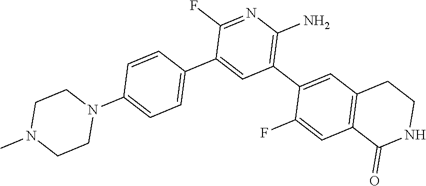 [SMILES: CN1CCN(c2ccc(-c3cc(-c4cc5c(cc4F)C(=O)NCC5)c(N)nc3F)cc2)CC1] | A | D |
| I-247 | 6-(2-amino-6-fluoro-5-(3- fluoro-4-(4- methylpiperazin-1- yl)phenyl)pyridin-3-yl)-7- fluoro-3,4- dihydroisoquinolin- 1(2H)-one | 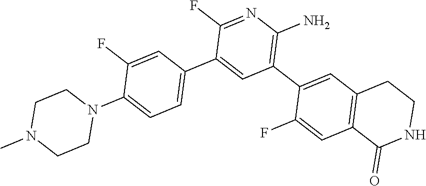 [SMILES: CN1CCN(c2ccc(-c3cc(-c4cc5c(cc4F)C(=O)NCC5)c(N)nc3F)cc2F)CC1] | A | D |
| I-248 | 6-(2-amino-6-fluoro-5-(4- (1-(2,2,2- trifluoroethyl)piperidin-4- yl)phenyl)pyridin-3-yl)-7- fluoro-3,4- dihydroisoquinolin- 1(2H)-one | 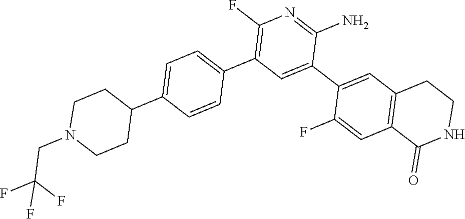 [SMILES: Nc1nc(F)c(-c2ccc(C3CCN(CC(F)(F)F)CC3)cc2)cc1-c1cc2c(cc1F)C(=O)NCC2] | B | D |
| I-249 | 6-(2-amino-6-fluoro-5-(4- (4-methylpiperazin-1-yl)- 3- (trifluoromethyl)phenyl)py- ridin-3-yl)-7-fluoro-3,4- dihydroisoquinolin- 1(2H)-one | 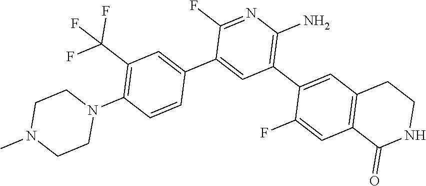 [SMILES: CN1CCN(c2ccc(-c3cc(-c4cc5c(cc4F)C(=O)NCC5)c(N)nc3F)cc2C(F)(F)F)CC1] | A | 381 |
| I-250 | 6-(2-amino-6-fluoro-5-(2- fluoro-5-nitro-4-((3R,5S)- 3,4,5-trimethylpiperazin- 1-yl)phenyl)pyriidn-3-yl)- 3,4-dihydroisoquinolin- 1(2H)-one formate | 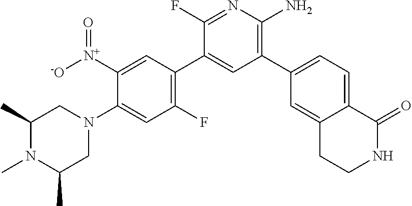 | B | D |
| I-251 | 6-(2-amino-6-fluoro-5-(4- (4-fluoro-1-(2- methoxyethyl)piperidin- 4-yl)phenyl)pyridin-3-yl)- 3,4-dihydroisoquinolin- 1(2H)-one | 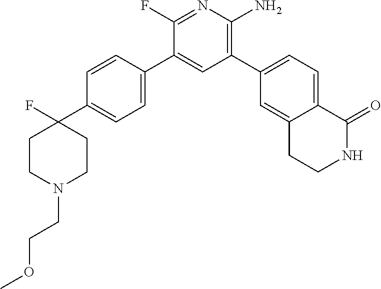 [SMILES: COCCN1CCC(F)(c2ccc(-c3cc(-c4ccc5c(c4)CCNC5=O)c(N)nc3F)cc2)CC1] | A | D |
| I-252 | 6-(2-amino-5-(2-chloro- 4-(4-methylpiperazin-1- yl)phenyl)-6- fluoropyridin-3-yl)-7- fluoro-3,4- dihydroisoquinolin- 1(2H)-one | 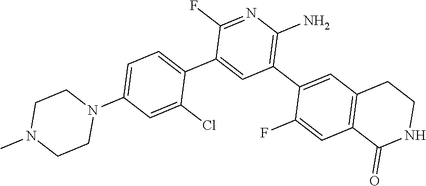 [SMILES: CN1CCN(c2ccc(-c3cc(-c4cc5c(cc4F)C(=O)NCC5)c(N)nc3F)c(Cl)c2)CC1] | A | D |
| I-253 | 6-(2-amino-6-fluoro-5-(1- methylindolin-5- yl)pyridin-3-yl)-3,4- dihydroisoquinolin- 1(2H)-one | 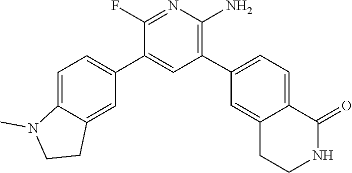 [SMILES: CN1CCc2cc(-c3cc(-c4ccc5c(c4)CCNC5=O)c(N)nc3F)ccc21] | B | D |
| I-254 | 6-(2-amino-6-fluoro-5-(2- methyl-1,2,3,4- tetrahydroisoquinolin-6- yl)pyridin-3-yl)-3,4- dihydroisoquinolin- 1(2H)-one | 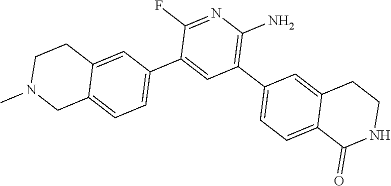 [SMILES: CN1CCc2cc(-c3cc(-c4ccc5c(c4)CCNC5=O)c(N)nc3F)ccc2C1] | B | D |
| I-255 | 6-(2-amino-6-fluoro-5- (1,2,3,4- tetrahydroisoquinolin-6- yl)pyridin-3-yl)-3,4- dihydroisoquinolin- 1(2H)-one 2,2,2- trifluoroacetate | 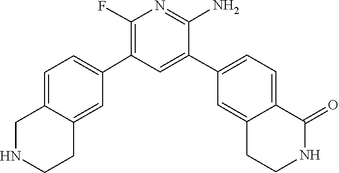 [SMILES: Nc1nc(F)c(-c2ccc3c(c2)CCNC3)cc1-c1ccc2c(c1)CCNC2=O] | A | D |
| I-256 | 6-(2-amino-5-(4-(1-(2,2- difluoroethyl)piperidin-4- yl)phenyl)-6- fluoropyridin-3-yl)-7- fluoro-3,4- dihydroisoquinolin- 1(2H)-one | 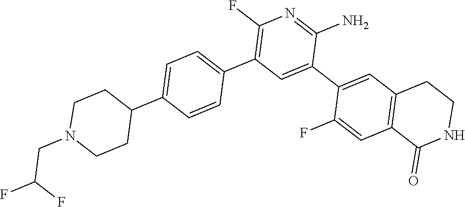 [SMILES: Nc1nc(F)c(-c2ccc(C3CCN(CC(F)F)CC3)cc2)cc1-c1cc2c(cc1F)C(=O)NCC2] | A | D |
| I-257 | 6-(2-amino-6-fluoro-5-(4- (morpholinomethyl)phenyl) pyridin-3-yl)-3,4- dihydroisoquinolin- 1(2H)-one | 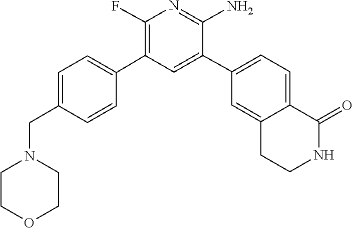 [SMILES: Nc1nc(F)c(-c2ccc(CN3CCOCC3)cc2)cc1-c1ccc2c(c1)CCNC2=O] | B | D |
| I-258 | 6-(2-amino-6-fluoro-5-(4- ((4-methylpiperazin-1- yl)methyl)phenyl)pyridin- 3-yl)-3,4- dihydroisoquinolin- 1(2H)-one | 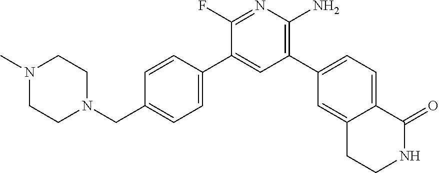 [SMILES: CN1CCN(Cc2ccc(-c3cc(-c4ccc5c(c4)CCNC5=O)c(N)nc3F)cc2)CC1] | B | D |
| I-259 | 6-(2-amino-6-fluoro-5-(4- (4-methylpiperazine-1- carbonyl)phenyl)pyridin- 3-yl)-3,4- dihydroisoquinolin- 1(2H)-one | 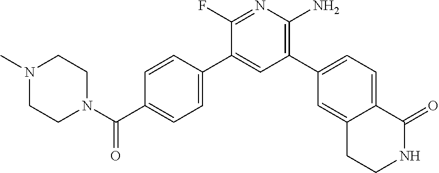 [SMILES: CN1CCN(C(=O)c2ccc(-c3cc(-c4ccc5c(c4)CCNC5=O)c(N)nc3F)cc2)CC1] | B | D |
| I-260 | 6-(2-amino-6-fluoro-5-(4- (1-(4-methylpiperazin-1- yl)cyclopropyl)phenyl)pyri- din-3-yl)-3,4- dihydroisoquinolin- 1(2H)-one | 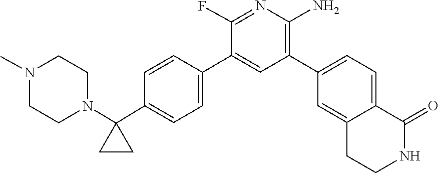 [SMILES: CN1CCN(C2(c3ccc(-c4cc(-c5ccc6c(c5)CCNC6=O)c(N)nc4F)cc3)CC2)CC1] | B | D |
| I-261 | 6-(2-amino-6-fluoro-5-(4- ((1-isopropylpiperidin-4- yl)oxy)-3- (trifluoromethyl)phenyl)py- ridin-3-yl)-3,4- dihydroisoquinolin- 1(2H)-one | 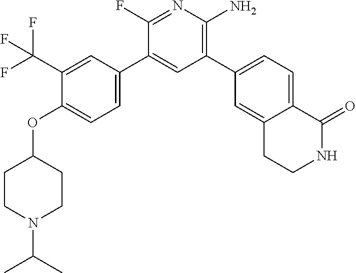 [SMILES: CC(C)N1CCC(Oc2ccc(-c3cc(-c4ccc5c(c4)CCNC5=O)c(N)nc3F)cc2C(F)(F)F)CC1] | B | D |
| I-262 | 6-(2-amino-6-fluoro-5-(4- ((1-(2- methoxyethyl)piperidin- 4-yl)oxy)-3- (trifluoromethyl)phenyl)py- ridin-3-yl)-3,4- dihydroisoquinolin- 1(2H)-one | 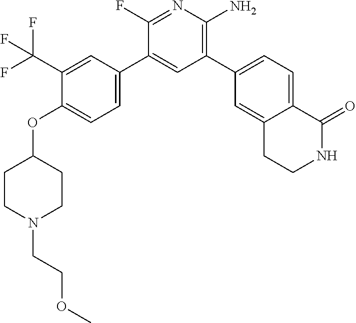 [SMILES: COCCN1CCC(Oc2ccc(-c3cc(-c4ccc5c(c4)CCNC5=O)c(N)nc3F)cc2C(F)(F)F)CC1] | A | D |
| I-263 | 6-(2-amino-6-fluoro-5-(4- (4-isopropylpiperazin-1- yl)-3- (trifluoromethyl)phenyl)py- ridin-3-yl)-7-fluoro-3,4- dihydroisoquinolin- 1(2H)-one | 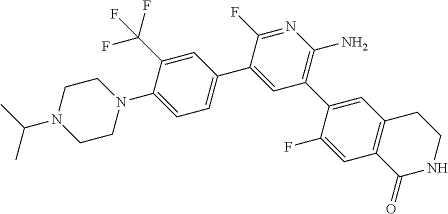 [SMILES: CC(C)N1CCN(c2ccc(-c3cc(-c4cc5c(cc4F)C(=O)NCC5)c(N)nc3F)cc2C(F)(F)F)CC1] | A | D |
| I-264 | 6-(2-amino-6-fluoro-5-(4- (4-(2- methoxyethyl)piperazin- 1-yl)-3- (trifluoromethyl)phenyl)py- riidn-3-yl)-7-fluoro-3,4- dihydroisoquinolin- 1(2H)-one | 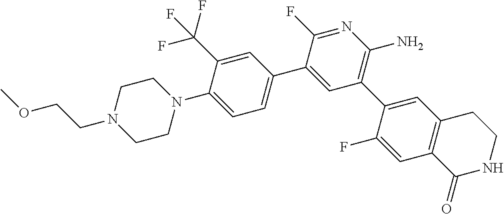 [SMILES: COCCN1CCN(c2ccc(-c3cc(-c4cc5c(cc4F)C(=O)NCC5)c(N)nc3F)cc2C(F)(F)F)CC1] | A | D |
| I-265 | 6-(2-amnio-6-fluoro-5-(3- (piperazin-1- ylmethyl)phenyl)pyridin- 3-yl)-3,4- dihydroisoquinolin- 1(2H)-one formate | 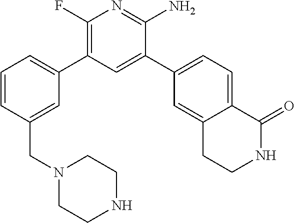 [SMILES: Nc1nc(F)c(-c2cccc(CN3CCNCC3)c2)cc1-c1ccc2c(c1)CCNC2=O] | B | D |
| I-266 | 6-(2-amino-6-fluoro-5-(2- (2-methoxyethyl)- 1,2,3,4- tetrahydroisoquinolin-6- yl)pyridin-3-yl)-3,4- dihydroisoquinolin- 1(2H)-one | 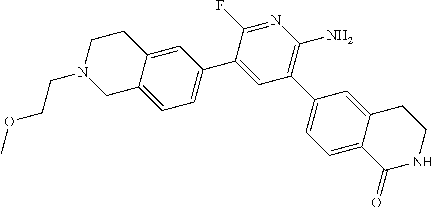 [SMILES: COCCN1CCc2cc(-c3cc(-c4ccc5c(c4)CCNC5=O)c(N)nc3F)ccc2C1] | B | D |
| I-267 | 6-(2-amino-6-fluoro-5-(4- ((1-isopropylpiperidin-4- yl)oxy)phenyl)pyridin-3- yl)-3,4- dihydroisoquinolin- 1(2H)-one | 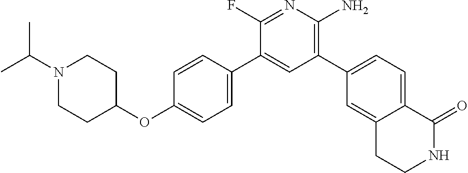 [SMILES: CC(C)N1CCC(Oc2ccc(-c3cc(-c4ccc5c(c4)CCNC5=O)c(N)nc3F)cc2)CC1] | A | D |
| I-268 | 6-(2-amino-6-fluoro-5-(3- fluoro-4-((1- isopropylpiperidin-4- yl)oxy)phenyl)pyridin-3- yl)-3,4- dihydroisoquinolin- 1(2H)-one | 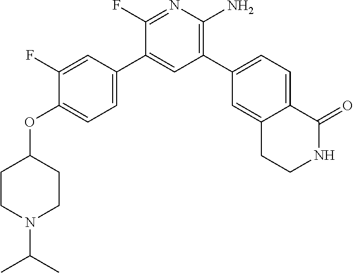 [SMILES: CC(C)N1CCC(Oc2ccc(-c3cc(-c4ccc5c(c4)CCNC5=O)c(N)nc3F)cc2F)CC1] | A | D |
| I-269 | 6-(2-amino-6-fluoro-5-(3- fluoro-4-((1-(2- methoxyethyl)piperidin- 4-yl)oxy)phenyl)pyridin- 3-yl)-3,4- dihydroisoquinolin- 1(2H)-one | 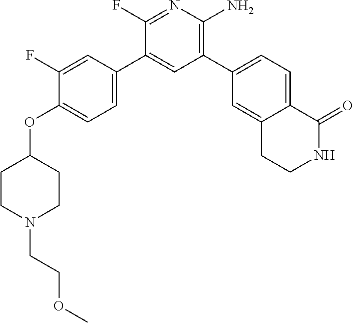 [SMILES: COCCN1CCC(Oc2ccc(-c3cc(-c4ccc5c(c4)CCNC5=O)c(N)nc3F)cc2F)CC1] | A | D |
| I-270 | 6-(2-amino-5-(2,3- difluoro-4-((1- isopropylpiperidin-4- yl)oxy)phenyl)-6- fluoropyridin-3-yl)-3,4- dihydroisoquinolin- 1(2H)-one | 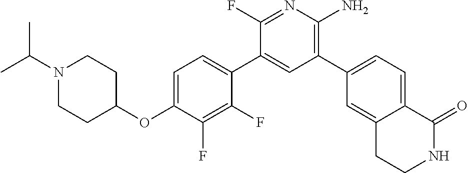 [SMILES: CC(C)N1CCC(Oc2ccc(-c3cc(-c4ccc5c(c4)CCNC5=O)c(N)nc3F)c(F)c2F)CC1] | B | D |
| I-271 | 6-(2-amino-5-(2,5- difluoro-4-((1- isopropylpiperidin-4- yl)oxy)phenyl)-6- fluoropyridin-3-yl)-3,4- dihydroisoquinolin- 1(2H)-one | 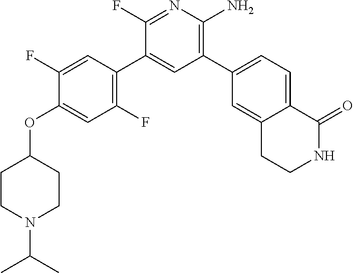 [SMILES: CC(C)N1CCC(Oc2cc(F)c(-c3cc(-c4ccc5c(c4)CCNC5=O)c(N)nc3F)cc2F)CC1] | B | D |
| I-272 | 6-(2-amino-6-fluoro-5-(4- ((1-isopropylpiperidin-4- yl)oxy)-2- methoxyphenyl)pyridin- 3-yl)-3,4- dihydroisoquinolin- 1(2H)-one | 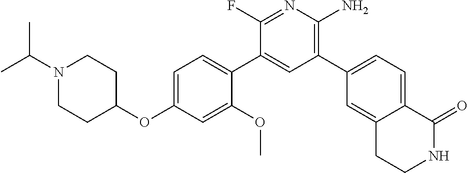 [SMILES: COc1cc(OC2CCN(C(C)C)CC2)ccc1-c1cc(-c2ccc3c(c2)CCNC3=O)c(N)nc1F] | A | D |
| I-273 | 6-(2-amino-6-fluoro-5-(4- ((1-isopropylpiperidin-4- yl)oxy)-3- methoxyphenyl)pyridin- 3-yl)-3,4- dihydroisoquinolin- 1(2H)-one | 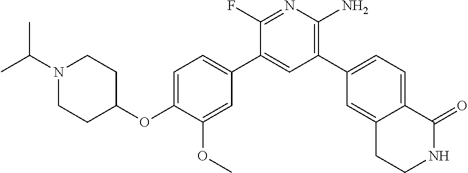 [SMILES: COc1cc(-c2cc(-c3ccc4c(c3)CCNC4=O)c(N)nc2F)ccc1OC1CCN(C(C)C)CC1] | A | D |
| I-274 | (S)-6-(2-amino-6-fluoro- 5-(4-((1- isopropylpiperidin-3- yl)oxy)phenyl)pyridin-3- yl)-3,4- dihydroisoquinolin- 1(2H)-one | 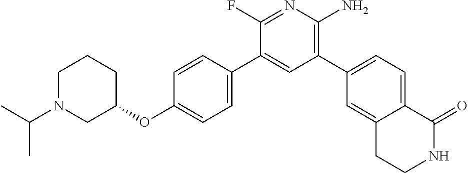 [SMILES: CC(C)N1CCC[C@H](Oc2ccc(-c3cc(-c4ccc5c(c4)CCNC5=O)c(N)nc3F)cc2)C1] | A | D |
| I-275 | 6-(2-amino-6-fluoro-5-(7- ((1-isopropylpiperidin-4- yl)oxy)benzo[d][1,3]dioxol- 4-yl)pyridin-3-yl)-3,4- dihydroisoquinolin- 1(2H)-one | 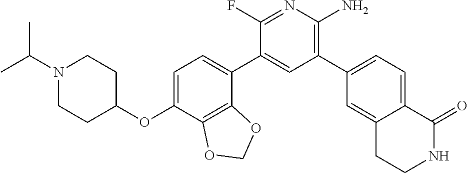 [SMILES: CC(C)N1CCC(Oc2ccc(-c3cc(-c4ccc5c(c4)CCNC5=O)c(N)nc3F)c3c2OCO3)CC1] | A | D |
| I-276 | 6-(6-amino-2-fluoro-5-(1- oxo-1,2,3,4- tetrahydroisoquinolin-6- yl)pyridin-3-yl)-3- ((methylamino)methyl)- 3,4-dihydroisoquinolin- 1(2H)-one | 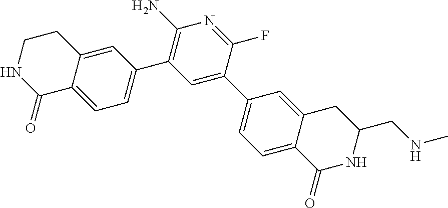 [SMILES: CNCC1Cc2cc(-c3cc(-c4ccc5c(c4)CCNC5=O)c(N)nc3F)ccc2C(=O)N1] | B | D |
| I-277 | 6-(2-amino-6-fluoro-5-(4- (4-methyl-1,4-diazepan- 1-yl)phenyl)pyridin-3-yl)- 3,4-dihydroisoquinolin- 1(2H)-one | 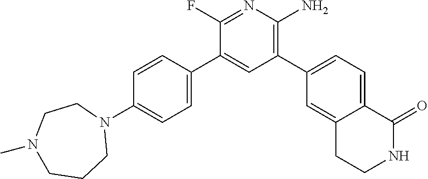 [SMILES: CN1CCCN(c2ccc(-c3cc(-c4ccc5c(c4)CCNC5=O)c(N)nc3F)cc2)CC1] | A | D |
| I-278 | 6-(2-amino-6-fluoro-5-(4- (4-isopropyl-3- methylpiperazin-1- yl)phenyl)pyridin-3-yl)- 3,4-dihydroisoquinolin- 1(2H)-one | 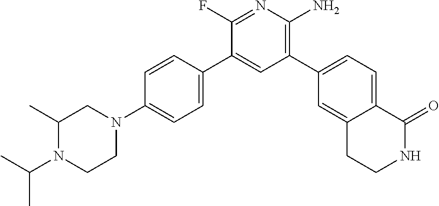 [SMILES: CC(C)N1CCN(c2ccc(-c3cc(-c4ccc5c(c4)CCNC5=O)c(N)nc3F)cc2)CC1C] | A | D |
| I-279 | 6-(2-amino-6-fluoro-5-(4- (4-isopropyl-1,4- diazepan-1- yl)phenyl)pyridin-3-yl)- 3,4-dihydroisoquinolin- 1(2H)-one | 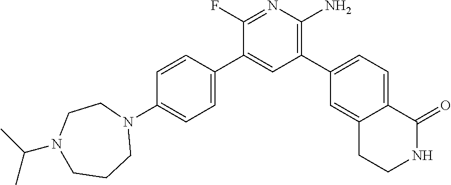 [SMILES: CC(C)N1CCCN(c2ccc(-c3cc(-c4ccc5c(c4)CCNC5=O)c(N)nc3F)cc2)CC1] | A | D |
| I-280 | 6-(2-amino-6-fluoro-5-(4- (hexahydropyrrolo[1,2- a]pyrazin-2(1H)- yl)phenyl)pyridin-3-yl)- 3,4-dihydroisoquinolin- 1(2H)-one | 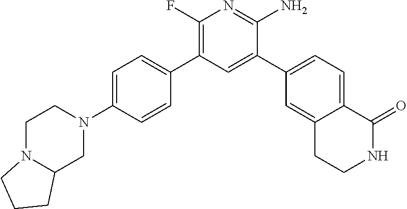 [SMILES: Nc1nc(F)c(-c2ccc(N3CCN4CCCC4C3)cc2)cc1-c1ccc2c(c1)CCNC2=O] | A | D |
| I-281 | 6-(2-amino-6-fluoro-5-(4- (hexahydro-1H- pyrrolo[1,2- a][1,4]diazepin-2(3H)- yl)phenyl)pyridin-3-yl)- 3,4-dihydroisoquinolin- 1(2H)-one | 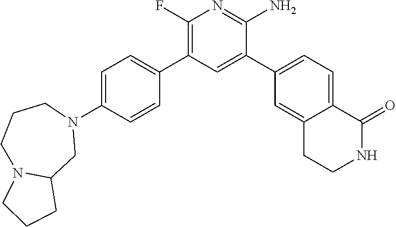 [SMILES: Nc1nc(F)c(-c2ccc(N3CCCN4CCCC4C3)cc2)cc1-c1ccc2c(c1)CCNC2=O] | A | D |
| I-282 | (S)-6-(2-amino-5-(4-(2,4- dimethylpiperazin-1- yl)phenyl)-6- fluoropyridin-3-yl)-3,4- dihydroisoquinolin- 1(2H)-one | 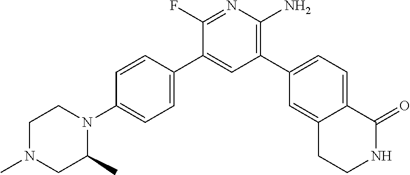 [SMILES: C[C@H]1CN(C)CCN1c1ccc(-c2cc(-c3ccc4c(c3)CCNC4=O)c(N)nc2F)cc1] | A | D |
| I-283 | 6-(2-amino-5-(4-(4- (dimethylamino)piperidin- 1-yl)phenyl)-6- fluoropyridin-3-yl)-3,4- dihydroisoquinolin- 1(2H)-one | 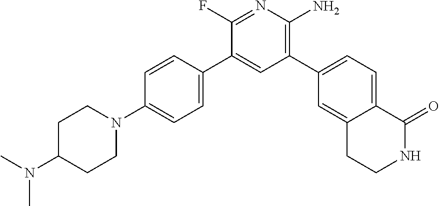 [SMILES: CN(C)C1CCN(c2ccc(-c3cc(-c4ccc5c(c4)CCNC5=O)c(N)nc3F)cc2)CC1] | A | D |
| I-284 | tert-butyl (2R,6S)-4-(4- (6-amino-2-fluoro-5-(1- oxo-1,2,3,4- tetrahydroisoquinolin-6- yl)pyridin-3-yl)phenyl)- 2,6-dimethylpiperazine- 1-carboxylate | 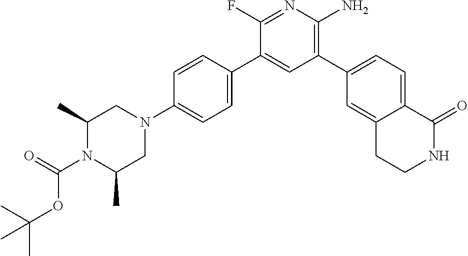 [SMILES: C[C@@H]1CN(c2ccc(-c3cc(-c4ccc5c(c4)CCNC5=O)c(N)nc3F)cc2)C[C@H](C)N1C(=O)OC(C)(C)C] | B | D |
| I-285 | 6-(2-amino-6-fluoro-5-(4- morpholinophenyl)pyridin- 3-yl)-3- ((methylamino)methyl)- 3,4-dihydroisoquinolin- 1(2H)-one | 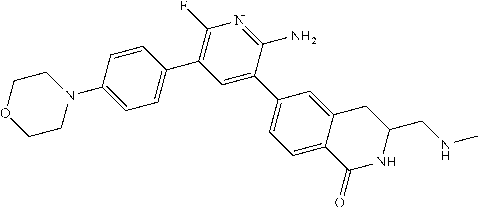 [SMILES: CNCC1Cc2cc(-c3cc(-c4ccc(N5CCOCC5)cc4)c(F)nc3N)ccc2C(=O)N1] | B | D |
| I-286 | 6-(2-amino-6-fluoro-5-(4- (4-isopropylpiperazin-1- yl)phenyl)pyridin-3-yl)-3- ((methylamino)methyl)- 3,4-dihydroisoquinolin- 1(2H)-one | 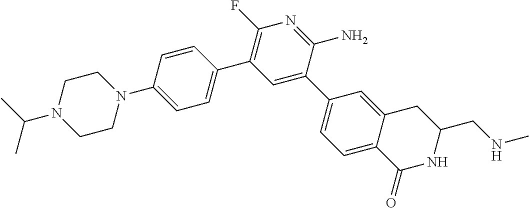 [SMILES: CNCC1Cc2cc(-c3cc(-c4ccc(N5CCN(C(C)C)CC5)cc4)c(F)nc3N)ccc2C(=O)N1] | A | D |
| I-287 | 6-(2-amino-6-fluoro-5-(4- (piperidin-4- yl)phenyl)pyridin-3-yl)-7- fluoro-3,4- dihydroisoquinolin-1(2H)- one | 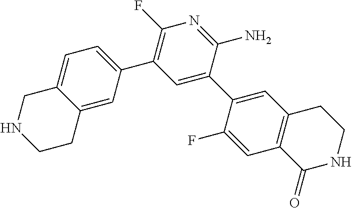 [SMILES: Nc1nc(F)c(-c2ccc3c(c2)CCNC3)cc1-c1cc2c(cc1F)C(=O)NCC2] | A | D |
| I-288 | 6-(2-amino-6-fluoro-5-(4- ((1-isopropylazetidin-3- yl)oxy)phenyl)pyridin-3- yl)-3,4- dihydroisoquinolin- 1(2H)-one | 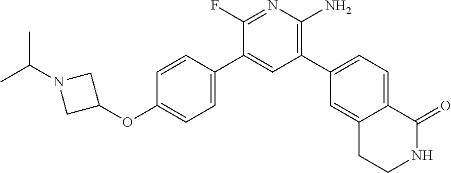 [SMILES: CC(C)N1CC(Oc2ccc(-c3cc(-c4ccc5c(c4)CCNC5=O)c(N)nc3F)cc2)C1] | A | D |
| I-289 | 5-(6-amino-2-fluoro-5-(7- fluoro-1-oxo-1,2,3,4- tetrahydroisoquinolin-6- yl)pyridin-3-yl)-2- (piperidin-4- yloxy)benzonitrile | 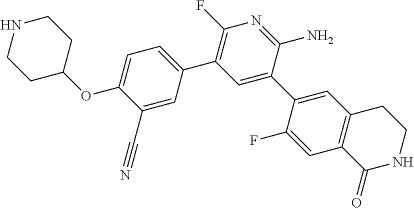 [SMILES: N#Cc1cc(-c2cc(-c3cc4c(cc3F)C(=O)NCC4)c(N)nc2F)ccc1OC1CCNCC1] | B | D |
| I-290 | 6-(5-(4-((1- acetylpiperidin-4-yl)oxy)- 3-fluorophenyl)-2-amino- 6-fluoropyridin-3-yl)-3,4- dihydroisoquinolin- 1(2H)-one | 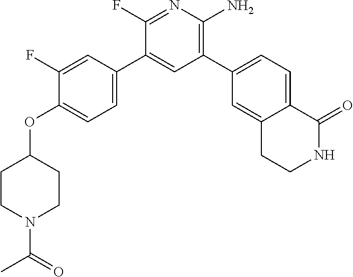 [SMILES: CC(=O)N1CCC(Oc2ccc(-c3cc(-c4ccc5c(c4)CCNC5=O)c(N)nc3F)cc2F)CC1] | A | A |
| I-291 | 2-((1-acetylpiperidin-4- yl)oxy)-5-(6-amino-2- fluoro-5-(7-fluoro-1-oxo- 1,2,3,4- tetrahydroisoquinolin-6- yl)pyridin-3- yl)benzonitrile | 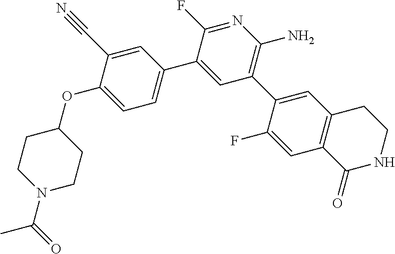 [SMILES: CC(=O)N1CCC(Oc2ccc(-c3cc(-c4cc5c(cc4F)C(=O)NCC5)c(N)nc3F)cc2C#N)CC1] | A | |
| I-292 | 5-(6-amino-2-fluoro-5-(7- fluoro-1-oxo-1,2,3,4- tetrahydroisoquinolin-6- yl)pyridin-3-yl)-2-((1- isopropylpiperidin-4- yl)oxy)benzonitrile | 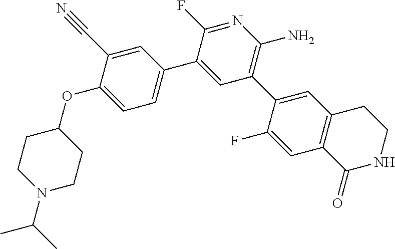 [SMILES: CC(C)N1CCC(Oc2ccc(-c3cc(-c4cc5c(cc4F)C(=O)NCC5)c(N)nc3F)cc2C#N)CC1] | A | D |
| I-293 | 5-(6-amino-2-fluoro-5-(7- fluoro-1-oxo-1,2,3,4- tetrahydroisoquinolin-6- yl)pyridin-3-yl)-2-((1- (2,2,2,- trifluoroethyl)piperidin-4- yl)oxy)benzonitrile | 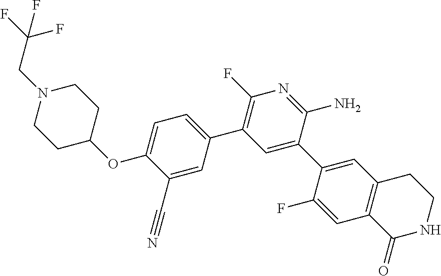 [SMILES: N#Cc1cc(-c2cc(-c3cc4c(cc3F)C(=O)NCC4)c(N)nc2F)ccc1OC1CCN(CC(F)(F)F)CC1] | B | D |
| I-294 | 6-(2-amino-6-fluoro-5-(4- (1-isopropyl-4- methoxypiperidin-4- yl)phenyl)pyridin-3-yl)- 3,4-dihydroisoquinolin- 1(2H)-one | 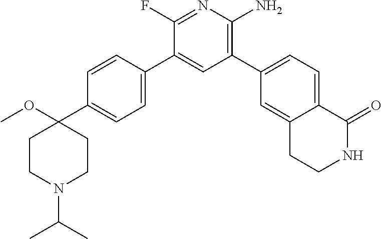 [SMILES: COC1(c2ccc(-c3cc(-c4ccc5c(c4)CCNC5=O)c(N)nc3F)cc2)CCN(C(C)C)CC1] | A | D |
| I-295 | 6-(2-amino-6-fluoro-5-(4- (1-isopropyl-1,2,3,6- tetrahydropyridin-4- yl)phenyl)pyridin-3-yl)-7- fluoro-3,4- dihydroisoquinolin- 1(2H)-one | 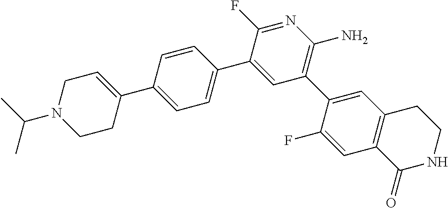 [SMILES: CC(C)N1CC=C(c2ccc(-c3cc(-c4cc5c(cc4F)C(=O)NCC5)c(N)nc3F)cc2)CC1] | A | D |
| I-296 | 6-(2-amino-5-(4-(1-ethyl- 1,2,3,6- tetrahydropyridin-4- yl)phenyl)-6- fluoropyridin-3-yl)-7- fluoro-3,4- dihydroisoquinolin- 1(2H)-one | 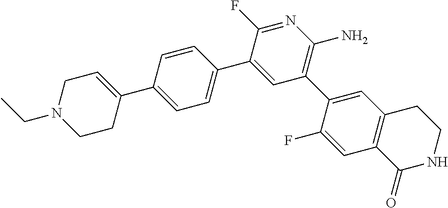 [SMILES: CCN1CC=C(c2ccc(-c3cc(-c4cc5c(cc4F)C(=O)NCC5)c(N)nc3F)cc2)CC1] | A | D |
| I-297 | 6-(2-amino-5-(4-(1- (cyclopropylmethyl)- 1,2,3,6- tetrahydropyridin-4- yl)phenyl)-6- fluoropyridin-3-yl)-7- fluoro-3,4- dihydroisoquinolin- 1(2H)-one | 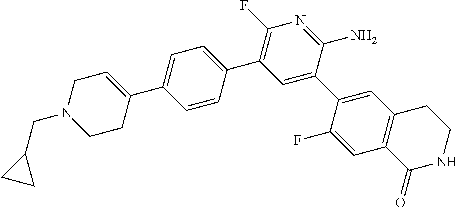 [SMILES: Nc1nc(F)c(-c2ccc(C3=CCN(CC4CC4)CC3)cc2)cc1-c1cc2c(cc1F)C(=O)NCC2] | A | D |
| I-298 | 6-(2-amino-5-(4- chlorophenyl)-6- fluoropyridin-3-yl)-3- ((methylamino)methyl)- 3,4-dihydroisoquinolin- 1(2H)-one | 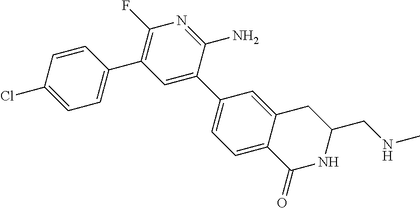 [SMILES: CNCC1Cc2cc(-c3cc(-c4ccc(Cl)cc4)c(F)nc3N)ccc2C(=O)N1] | B | D |
| I-299 | 6-(2-amino-6-fluoro-5-(2- isopropyl-1,2,3,4- tetrahydroisoquinolin-6- yl)pyridin-3-yl)-7-fluoro- 3,4-dihydroisoquinolin- 1(2H)-one | 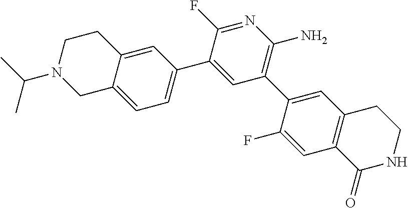 [SMILES: CC(C)N1CCc2cc(-c3cc(-c4cc5c(cc4F)C(=O)NCC5)c(N)nc3F)ccc2C1] | A | D |
| I-300 | 6-(2-amino-6-fluoro-5-(2- (2-methoxyethyl)- 1,2,3,4- tetrahydroisoquinolin-6- yl)pyridin-3-yl)-7-fluoro- 3,4-dihydroisoquinolin- 1(2H)-one | 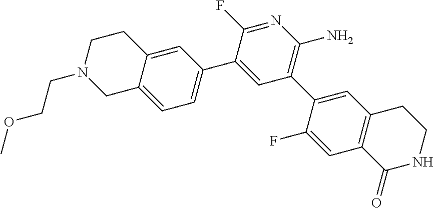 [SMILES: COCCN1CCc2cc(-c3cc(-c4cc5c(cc4F)C(=O)NCC5)c(N)nc3F)ccc2C1] | A | D |
| I-301 | 6-(2-amino-6-fluoro-5-(4- ((1R,5S)-3-isopropyl-3- azabicyclo[3.1.0]hexan- 1-yl)phenyl)pyridin-3-yl)- 8-fluoro-3,4- dihydroisoquinolin- 1(2H)-one | 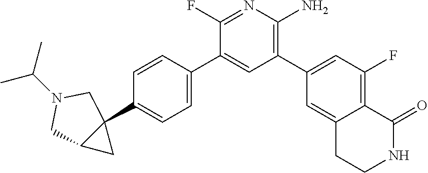 [SMILES: CC(C)N1C[C@H]2C[C@@]2(c2ccc(-c3cc(-c4cc(F)c5c(c4)CCNC5=O)c(N)nc3F)cc2)C1] | A | D |
| I-302 | 6-(2-amino-6-fluoro-5-(4- (1-isopropylpiperidin-4- yl)phenyl)pyridin-3-yl)-8- fluoro-3,4- dihydroisoquinolin- 1(2H)-one | 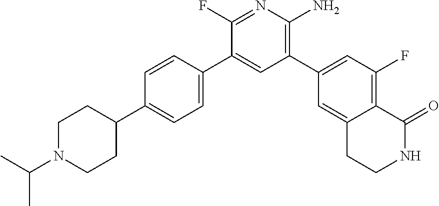 [SMILES: CC(C)N1CCC(c2ccc(-c3cc(-c4cc(F)c5c(c4)CCNC5=O)c(N)nc3F)cc2)CC1] | A | C |
| I-303 | 5-(6-amino-2-fluoro-5-(7- fluoro-1-oxo-1,2,3,4- tetrahydroisoquinolin-6- yl)pyridin-3-yl)-2-((1-(2- methoxyethyl)piperidin- 4-yl)oxy)benzonitrile | 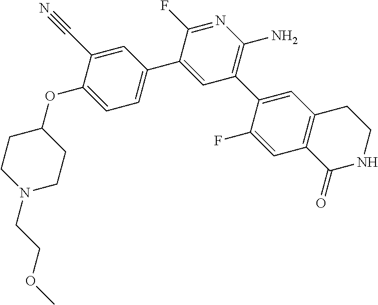 [SMILES: COCCN1CCC(Oc2ccc(-c3cc(-c4cc5c(cc4F)C(=O)NCC5)c(N)nc3F)cc2C#N)CC1] | B | D |
| I-304 | 6-(5-(4-((1- acetylpiperidin-4-yl)oxy)- 2-methoxyphenyl)-2- amino-6-fluoropyridin-3- yl)-3,4- dihydroisoquinolin- 1(2H)-one | 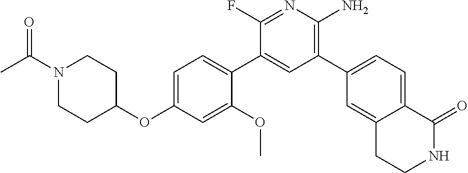 [SMILES: COc1cc(OC2CCN(C(C)=O)CC2)ccc1-c1cc(-c2ccc3c(c2)CCNC3=O)c(N)nc1F] | A | D |
| I-305 | 6-(5-(4-((1- acetylpiperidin-4-yl)oxy)- 3-methoxyphenyl)-2- amino-6-fluoropyridin-3- yl)-3,4- dihydroisoquinolin- 1(2H)-one | 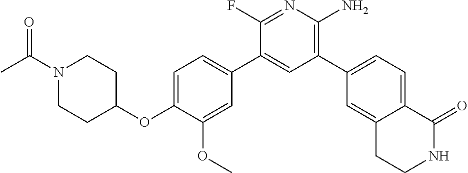 [SMILES: COc1cc(-c2cc(-c3ccc4c(c3)CCNC4=O)c(N)nc2F)ccc1OC1CCN(C(C)=O)CC1] | A | D |
| I-306 | 6-(5-(7-((1- acetylpiperidin-4- yl)oxy)benzo[d][1,3]dioxol- 4-yl)-2-amino-6- fluoropyridin-3-yl)-3,4- dihydroisoquinolin- 1(2H)-one | 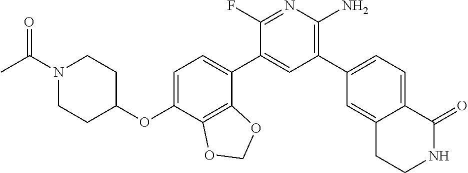 [SMILES: CC(=O)N1CCC(Oc2ccc(-c3cc(-c4ccc5c(c4)CCNC5=O)c(N)nc3F)c3c2OCO3)CC1] | A | D |
| I-307 | 6-(2-amino-5-(4-(4- (cyclopropylmethyl)piper- azin-1-yl)phenyl)-6- fluoropyridin-3-yl)-8- fluoro-3,4- dihydroisoquinolin- 1(2H)-one | 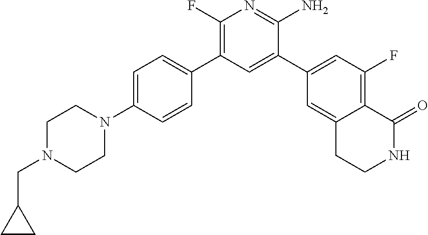 [SMILES: Nc1nc(F)c(-c2ccc(N3CCN(CC4CC4)CC3)cc2)cc1-c1cc(F)c2c(c1)CCNC2=O] | A | D |
| I-308 | 6-(2-amino-5-(4-(1- (cyclopropylmethyl)piperi- din-4-yl)phenyl)-6- fluoropyridin-3-yl)-8- fluoro-3,4- dihydroisoquinolin- 1(2H)-one | 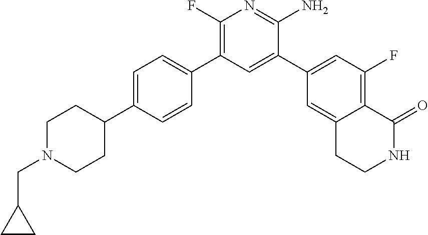 [SMILES: Nc1nc(F)c(-c2ccc(C3CCN(CC4CC4)CC3)cc2)cc1-c1cc(F)c2c(c1)CCNC2=O] | A | D |
| I-309 | 6-(2-amino-6-fluoro-5-(4- morpholinophenyl)pyridin- 3-yl)-8-fluoro-3,4- dihydroisoquinolin- 1(2H)-on | 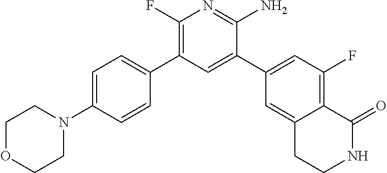 [SMILES: Nc1nc(F)c(-c2ccc(N3CCOCC3)cc2)cc1-c1cc(F)c2c(c1)CCNC2=O] | A | D |
| I-310 | 6-(2-amino-6-fluoro-5-(4- (4-isopropylpiperazin-1- yl)phenyl)pyridin-3-yl)-3- ((dimethylamino)methyl)- 3,4-dihydroisoquinolin- 1(2H)-one | 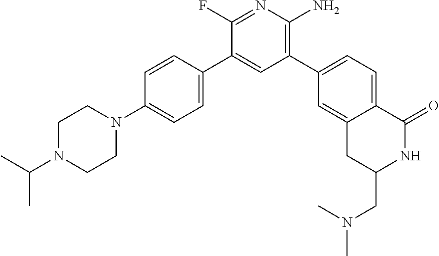 [SMILES: CC(C)N1CCN(c2ccc(-c3cc(-c4ccc5c(c4)CC(CN(C)C)NC5=O)c(N)nc3F)cc2)CC1] | B | D |
| I-311 | 6-(2-amino-6-fluoro-5-(4- morpholinophenyl)pyridin- 3-yl)-3- ((dimethylamino)methyl)- 3,4-dihydroisoquinolin- 1(2H)-one | 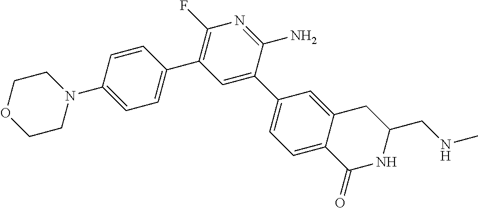 [SMILES: CNCC1Cc2cc(-c3cc(-c4ccc(N5CCOCC5)cc4)c(F)nc3N)ccc2C(=O)N1] | B | D |
| I-312 | 6-(2-amino-5-(4- chlorophenyl)-6- fluoropyridin-3-yl)-3- ((dimethylamino)methyl)- 3,4-dihydroisoquinolin- 1(2H)-one | 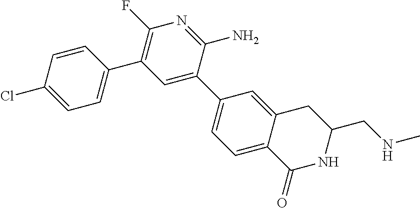 [SMILES: CNCC1Cc2cc(-c3cc(-c4ccc(Cl)cc4)c(F)nc3N)ccc2C(=O)N1] | C | D |
| I-313 | 6-(6-amino-2-fluoro-2′- methyl-[3,4′-bipyridin]-5- yl)-7-fluoro-3,4- dihydroisoquinolin- 1(2H)-one | 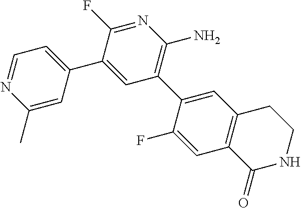 [SMILES: Cc1cc(-c2cc(-c3cc4c(cc3F)C(=O)NCC4)c(N)nc2F)ccn1] | B | D |
| I-314 | 6-(6-amino-2′- (cyclopropylmethoxy)-2- fluoro-[3,4′-bipyridin]-5- yl)-7-fluoro-3,4- dihydroisoquinolin- 1(2H)-one | 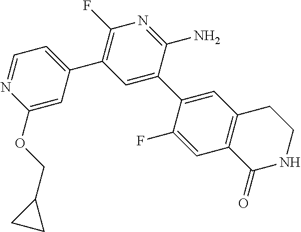 [SMILES: Nc1nc(F)c(-c2ccnc(OCC3CC3)c2)cc1-c1cc2c(cc1F)C(=O)NCC2] | C | D |
| I-315 | 6-(2-amino-5-(3- (cyclopropylmethoxy)phe- nyl)-6-fluoropyridin-3-yl)- 7-fluoro-3,4- dihydroisoquinolin- 1(2H)-one | 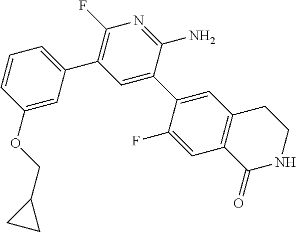 [SMILES: Nc1nc(F)c(-c2cccc(OCC3CC3)c2)cc1-c1cc2c(cc1F)C(=O)NCC2] | C | D |
| I-316 | 6-(2-amino-5-(4- (cyclopropylmethoxy)phe- nyl)-6-fluoropyridin-3-yl)- 7-fluoro-3,4- dihydroisoquinolin- 1(2H)-one | 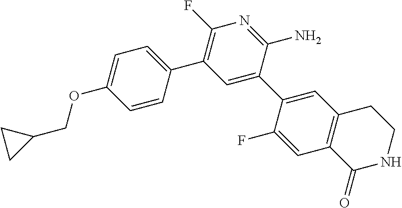 [SMILES: Nc1nc(F)c(-c2ccc(OCC3CC3)cc2)cc1-c1cc2c(cc1F)C(=O)NCC2] | B | D |
| I-317 | 6-(2-amino-5-(4-((1-(2- cyclopropylacetyl)azetidin- 3-yl)oxy)phenyl)-6- fluoropyridin-3-yl)-3,4- dihydroisoquinolin- 1(2H)-one | 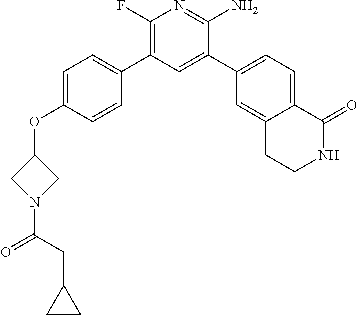 [SMILES: Nc1nc(F)c(-c2ccc(OC3CN(C(=O)CC4CC4)C3)cc2)cc1-c1ccc2c(c1)CCNC2=O] | A | D |
| I-318 | 6-(2-amino-6-fluoro-5-(4- ((1-(oxetan-3-yl)azetidin- 3-yl)oxy)phenyl)pyridin- 3 yl)-3,4- dihydroisoquinolin- 1(2H)-one | 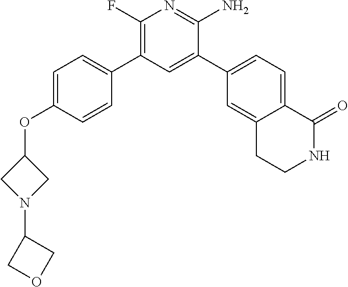 [SMILES: Nc1nc(F)c(-c2ccc(OC3CN(C4COC4)C3)cc2)cc1-c1ccc2c(c1)CCNC2=O] | B | D |
| I-319 | 6-(2-amino-5-(4- ((3R,5S)-3,5- dimethylpiperazin-1- yl)phenyl)-6- fluoropyridin-3-yl)-3,4- dihydroisoquinolin- 1(2H)-one | 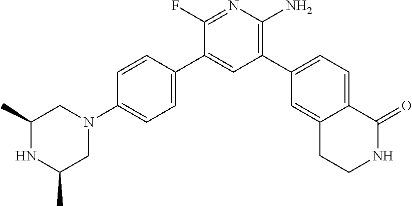 [SMILES: C[C@@H]1CN(c2ccc(-c3cc(-c4ccc5c(c4)CCNC5=O)c(N)nc3F)cc2)C[C@H](C)N1] | A | D |
| I-320 | 6-(2-amino-5-(4- ((1S,5R)-3- (cyclopropylmethyl)-3- azabicyclo[3.1.0]hexan- 1-yl)phenyl)-6- fluoropyridin-3-yl)-8- fluoro-3,4- dihydroisoquinolin- 1(2H)-one formate | 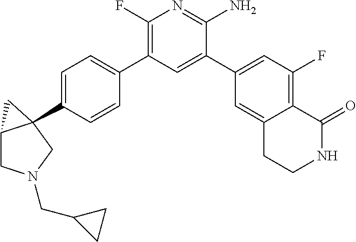 [SMILES: Nc1nc(F)c(-c2ccc([C@]34C[C@H]3CN(CC3CC3)C4)cc2)cc1-c1cc(F)c2c(c1)CCNC2=O] | A | C |
| I-321 | 6-(2-amino-5-(4-((1- (cyclopropylmethyl)azeti- din-3-yl)oxy)phenyl)-6- fluoropyridin-3-yl)-3,4- dihydroisoquinolin- 1(2H)-one | 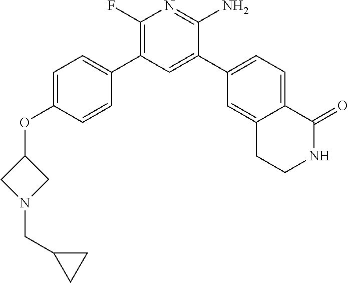 [SMILES: Nc1nc(F)c(-c2ccc(OC3CN(CC4CC4)C3)cc2)cc1-c1ccc2c(c1)CCNC2=O] | A | D |
| I-322 | 6-(2-amino-5-(4-((1- (cyclopropylmethyl)piperi- din-4-yl)oxy)phenyl)-6- fluoropyridin-3-yl)-3,4- dihydroisoquinoin- 1(2H)-one | 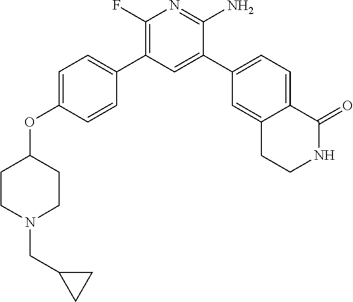 [SMILES: Nc1nc(F)c(-c2ccc(OC3CCN(CC4CC4)CC3)cc2)cc1-c1ccc2c(c1)CCNC2=O] | A | D |
| I-323 | 6-(2-amino-5-(4-((1-(2- cyclopropylacetyl)piperidin- 4-yl)oxy)phenyl)-6- fluoropyridin-3-yl)-7- fluoro-3,4- dihydroisoquinolin- 1(2H)-one | 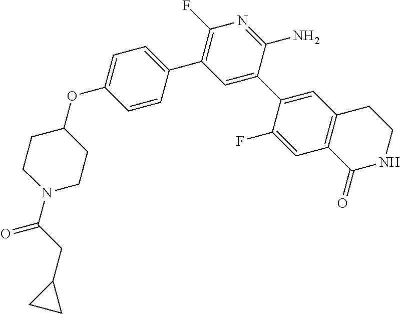 [SMILES: Nc1nc(F)c(-c2ccc(OC3CCN(C(=O)CC4CC4)CC3)cc2)cc1-c1cc2c(cc1F)C(=O)NCC2] | A | D |
| I-324 | 6-(2-amino-5-(4-((1- (cyclopropylmethyl)piperi- din-4-yl)oxy)phenyl)-6- fluoropyridin-3-yl)-7- fluoro-3,4- dihydroisoquinolin- 1(2H)-one | 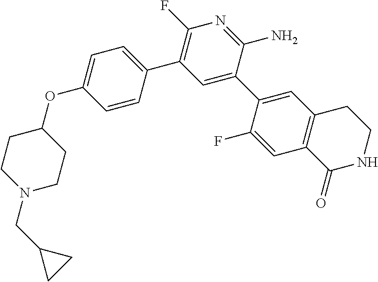 [SMILES: Nc1nc(F)c(-c2ccc(OC3CCN(CC4CC4)CC3)cc2)cc1-c1cc2c(cc1F)C(=O)NCC2] | A | D |
| I-325 | 6-(2-amino-6-fluoro-5-(4- ((1-(oxetan-3- yl)piperiidn-4- yl)oxy)phenyl)pyridin-3- yl)-7-fluoro-3,4- dihydroisoqinolin- 1(2H)-one | 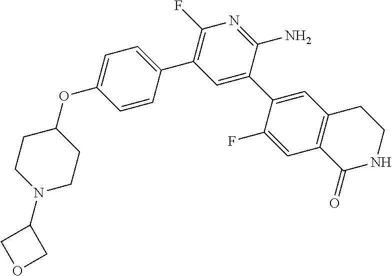 [SMILES: Nc1nc(F)c(-c2ccc(OC3CCN(C4COC4)CC3)cc2)cc1-c1cc2c(cc1F)C(=O)NCC2] | B | D |
| I-326 | 6-(5-(4-((1- acetylacetidin-3- yl)oxy)phenyl)-2-amino- 6-fluoropyridin-3-yl)-3,4- dihydroisoquinolin- 1(2H)-one | 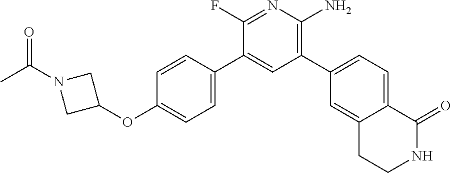 [SMILES: CC(=O)N1CC(Oc2ccc(-c3cc(-c4ccc5c(c4)CCNC5=O)c(N)nc3F)cc2)C1] | A | D |
| I-327 | 6-(5-(4-((1- acetylpiperidin-4- yl)oxy)phenyl)-2-amino- 6-fluoropyridin-3-yl)-7- fluoro-3,4- dihydroisoquinolin- 1(2H)-one | 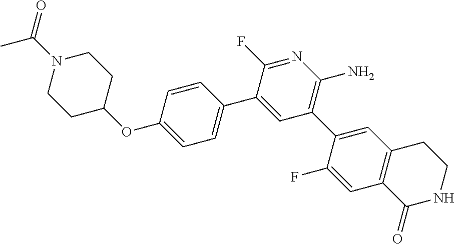 [SMILES: CC(=O)N1CCC(Oc2ccc(-c3cc(-c4cc5c(cc4F)C(=O)NCC5)c(N)nc3F)cc2)CC1] | A | D |
| I-328 | 6-(2-amino-6-fluoro-5-(2- isopropyl-1,2,3,4- tetrahydroisoquinolin-6- yl)pyridin-3-yl)-3,4- dihydroisoquinolin- 1(2H)-one | 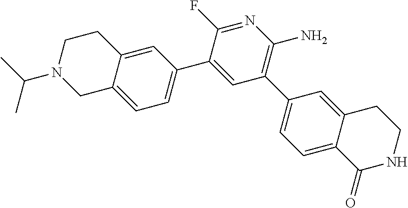 [SMILES: CC(C)N1CCc2cc(-c3cc(-c4ccc5c(c4)CCNC5=O)c(N)nc3F)ccc2C1] | B | D |
| I-329 | 6-(2-amino-6-fluoro-5-(3- ((4-(2- methoxyethyl)piperazin- 1- yl)methyl)phenyl)pyridin- 3-yl)-3,4- dihydroisoquinolin- 1(2H)-one | 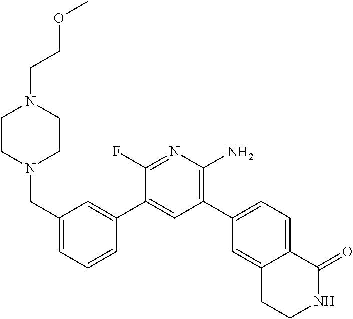 [SMILES: COCCN1CCN(Cc2cccc(-c3cc(-c4ccc5c(c4)CCNC5=O)c(N)nc3F)c2)CC1] | C | D |
| I-330 | 6-(2-amino-6-fluoro-5-(3- ((4-isopropylpiperazin-1- yl)methyl)phenyl)pyridin- 3-yl)-3,4- dihydroisoquinolin- 1(2H)-one | 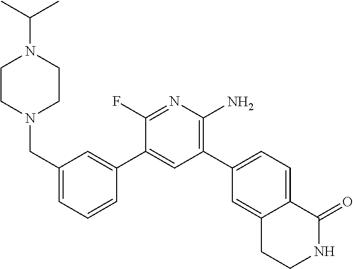 [SMILES: CC(C)N1CCN(Cc2cccc(-c3cc(-c4ccc5c(c4)CCNC5=O)c(N)nc3F)c2)CC1] | B | D |
| I-331 | 6-(2-amino-6-fluoro-5-(3- (4-methylpiperazine-1- carbonyl)phenyl)pyridin- 3-yl)-3,4- dihydroisoquinolin- 1(2H)-one | 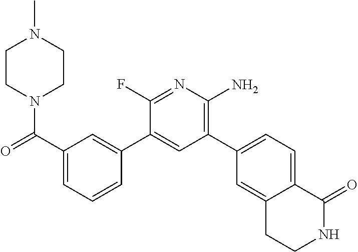 [SMILES: CN1CCN(C(=O)c2cccc(-c3cc(-c4ccc5c(c4)CCNC5=O)c(N)nc3F)c2)CC1] | C | D |
| I-332 | 6-(2-amino-5-(3-amino- 4-(4-methylpiperazin-1- yl)phenyl)-6- fluoropyridin-3-yl)-3,4- dihydroisoquinolin- 1(2H)-one | 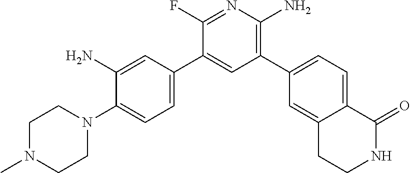 [SMILES: CN1CCN(c2ccc(-c3cc(-c4ccc5c(c4)CCNC5=O)c(N)nc3F)cc2N)CC1] | A | D |
| I-333 | 6,6′-(2-amino-6- fluoropyridine-3,5- diyl)bis(3,4- dihydroisoquinolin- 1(2H)-one) | 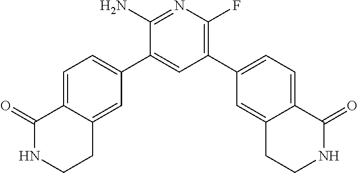 [SMILES: Nc1nc(F)c(-c2ccc3c(c2)CCNC3=O)cc1-c1ccc2c(c1)CCNC2=O] | A | D |
| I-334 | 6-(2-amino-6-fluoro-5- (1,2,3,4- tetrahydroisoquinolin-7- yl)pyridin-3-yl)-3,4- dihydroisoquinolin- 1(2H)-one 2,2,2- trifluoroacetate | 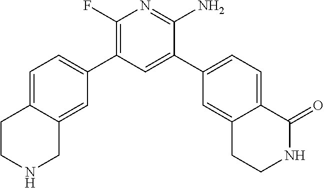 [SMILES: Nc1nc(F)c(-c2ccc3c(c2)CNCC3)cc1-c1ccc2c(c1)CCNC2=O] | B | D |
| I-335 | 6-(2-amino-6-fluoro-5-(2- isopropyl-1,2,3,4- tetrahydroisoquinolin-7- yl)pyridin-3-yl)-3,4- dihydroisoquinolin- 1(2H)-one | 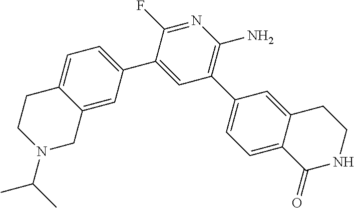 [SMILES: CC(C)N1CCc2ccc(-c3cc(-c4ccc5c(c4)CCNC5=O)c(N)nc3F)cc2C1] | B | D |
| I-336 | 6-(2-amino-6-fluoro-5-(4- (4-isopropylpiperazin-1- yl)phenyl)pyridin-3-yl)-8- fluoro-3,4- dihydroisoquinolin- 1(2H)-one | 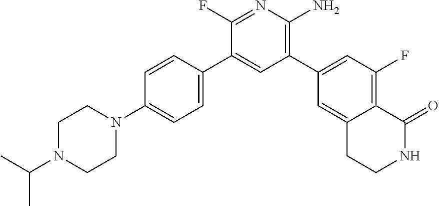 [SMILES: CC(C)N1CCN(c2ccc(-c3cc(-c4cc(F)c5c(c4)CCNC5=O)c(N)nc3F)cc2)CC1] | A | C |
| I-337 | 6-(2-amino-5-(4-((1-(2- cyclopropylacetyl)piperidin- 4-yl)oxy)phenyl)-6- fluoropyridin-3-yl)-3,4- dihydroisoquinolin- 1(2H)-one | 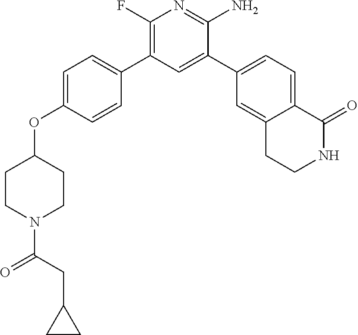 [SMILES: Nc1nc(F)c(-c2ccc(OC3CCN(C(=O)CC4CC4)CC3)cc2)cc1-c1ccc2c(c1)CCNC2=O] | B | D |
| I-338 | 6-(2-amino-6-fluoro-5-(4- ((1-(oxetan-3- yl)piperidin-4- yl)oxy)phenyl)pyridin-3- yl)-3,4- dihydroisoquinolin- 1(2H)-one | 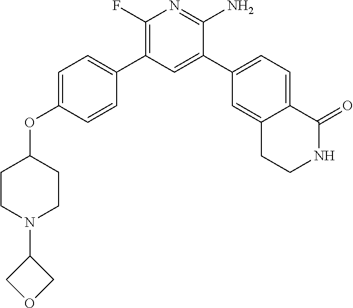 [SMILES: Nc1nc(F)c(-c2ccc(OC3CCN(C4COC4)CC3)cc2)cc1-c1ccc2c(c1)CCNC2=O] | B | D |
| I-339 | 6-(2-amino-5-(4- (azetidin-3- yloxy)phenyl)-6- fluoropyridin-3-yl)-7- fluoro-3,4- dihydroisoquinolin- 1(2H)-one 2,2,2- trifluoroacetate | 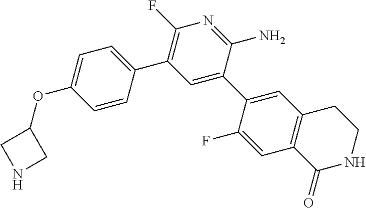 [SMILES: Nc1nc(F)c(-c2ccc(OC3CNC3)cc2)cc1-c1cc2c(cc1F)C(=O)NCC2] | B | D |
| I-340 | 6-(2-amino-6-fluoro-5-(4- (3-fluoroazetidin-3- yl)phenyl)pyridin-3-yl)- 3,4-dihydroisoquinolin- 1(2H)-one 2,2,2- trifluoroacetate | 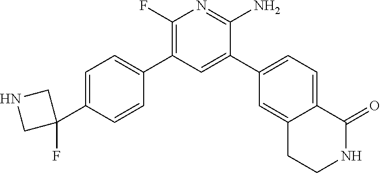 [SMILES: Nc1nc(F)c(-c2ccc(C3(F)CNC3)cc2)cc1-c1ccc2c(c1)CCNC2=O] | A | D |
| I-341 | 6-(5-(4-((1- acetylazetidin-3- yl)oxy)phenyl)-2-amino- 6-fluoropyridin-3-yl)-7- fluoro-3,4- dihydroisoquinolin- 1(2H)-one | 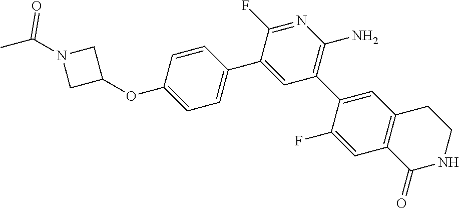 [SMILES: CC(=O)N1CC(Oc2ccc(-c3cc(-c4cc5c(cc4F)C(=O)NCC5)c(N)nc3F)cc2)C1] | B | D |
| I-342 | 6-(5-(4-(1-acetyl-3- fluoroazetidin-3- yl)phenyl)-2-amino-6- fluoropyridin-3-yl)-3,4- dihydroisoquinolin- 1(2H)-one | 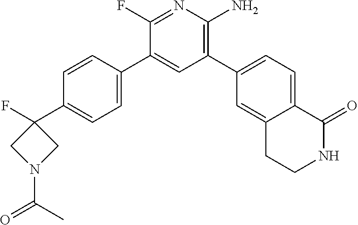 [SMILES: CC(=O)N1CC(F)(c2ccc(-c3cc(-c4ccc5c(c4)CCNC5=O)c(N)nc3F)cc2)C1] | A | D |
| I-343 | 6-(2-amino-6-fluoro-5-(4- ((1R,5S)-3-methyl-3- azabicyclo[3.1.0]hexan- 1-yl)phenyl)pyridin-3-yl)- 7-fluoro-3,4- dihydroisoquinolin- 1(2H)-one | 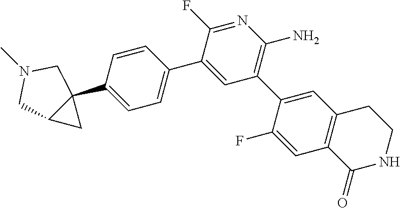 [SMILES: CN1C[C@H]2C[C@@]2(c2ccc(-c3cc(-c4cc5c(cc4F)C(=O)NCC5)c(N)nc3F)cc2)C1] | A | D |
| I-344 | 6-(2-amino-6-fluoro-5-(4- ((1S,5R)-3-methyl-3- azabicyclo[3.1.0]hexan- 1-yl)phenyl)pyridin-3-yl)- 7-fluoro-3,4- dihydroisoquinolin- 1(2H)-one | 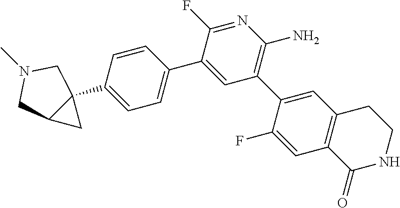 [SMILES: CN1C[C@@H]2C[C@]2(c2ccc(-c3cc(-c4cc5c(cc4F)C(=O)NCC5)c(N)nc3F)cc2)C1] | A | D |
| I-345 | 6-(2-amino-5-(2,3- difluoro-4-(4- isopropylpiperazin-1- yl)phenyl)-6- fluoropyridin-3-yl)-3,4- dihydroisoquinolin- 1(2H)-one | 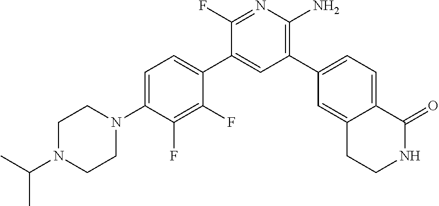 [SMILES: CC(C)N1CCN(c2ccc(-c3cc(-c4ccc5c(c4)CCNC5=O)c(N)nc3F)c(F)c2F)CC1] | A | D |
| I-346 | 6-(2-amino-5-(4-(1,1- dioxidothiomorpholino)phe- nyl)-6-fluoropyridin-3- yl)-7-fluoro-3,4- dihydroisoquinolin- 1(2H)-one | 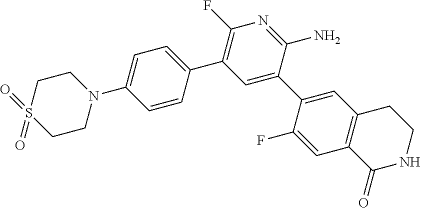 [SMILES: Nc1nc(F)c(-c2ccc(N3CCS(=O)(=O)CC3)cc2)cc1-c1cc2c(cc1F)C(=O)NCC2] | A | D |
| I-347 | 6-(2-amino-6-fluoro-5-(4- thiomorpholinophenyl)py- ridin-3-yl)-7-fluoro-3,4- dihydroisoquinolin- 1(2H)-one | 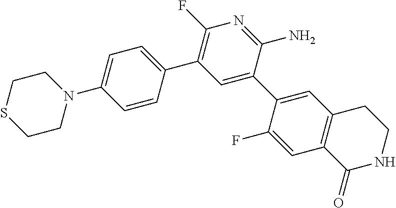 [SMILES: Nc1nc(F)c(-c2ccc(N3CCSCC3)cc2)cc1-c1cc2c(cc1F)C(=O)NCC2] | A | D |
| I-348 | (3aS,4S,6aR)-4-(5-(4-(4- (6-amino-2-fluoro-5-(1- oxo-1,2,3,4- tetrahydroisoquinolin-6- yl)pyridin-3- yl)phenoxy)piperidin-1- yl)-5- oxopentyl)tetrahydro-1H- thieno[3,4-d]imidazol- 2(3H)-one | 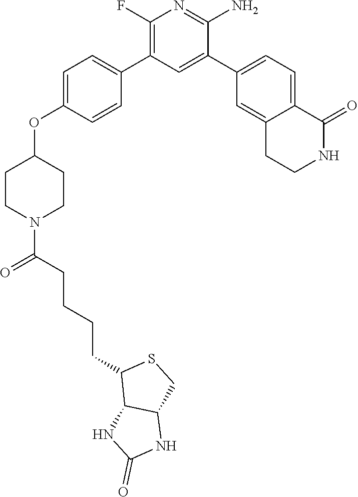 | A | D |
| I-349 | 6-(2-amino-6-fluoro-5-(4- (4-isopropylpiperazin-1- yl)phenyl)pyriidn-3-yl)-7- chloro-3,4- dihydroisoquinolin- 1(2H)-one | 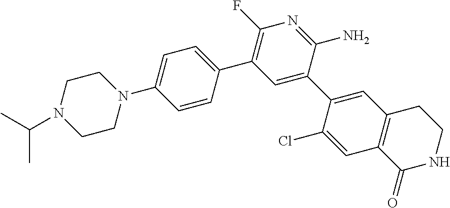 [SMILES: CC(C)N1CCN(c2ccc(-c3cc(-c4cc5c(cc4Cl)C(=O)NCC5)c(N)nc3F)cc2)CC1] | A | D |
| I-350 | 6-(2-amino-6-fluoro-5-(4- (1-(2- methoxyethyl)piperidin- 4-yl)phenyl)pyridin-3-yl)- 8-fluoro-3,4- dihydroisoquinolin- 1(2H)-one | 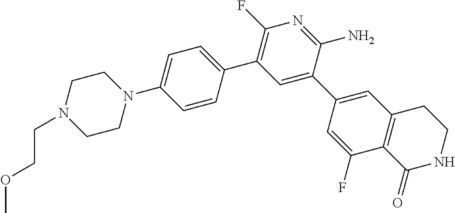 [SMILES: COCCN1CCN(c2ccc(-c3cc(-c4cc(F)c5c(c4)CCNC5=O)c(N)nc3F)cc2)CC1] | A | C |
| I-351 | (R)-6-(2-amino-6-fluoro- 5-(4-(2- isopropylmorpholino)phe- nyl)pyridin-3-yl)-8-fluoro- 3,4-dihydroisoquinolin- 1(2H)-one | 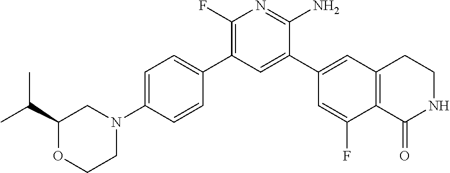 [SMILES: CC(C)[C@H]1CN(c2ccc(-c3cc(-c4cc(F)c5c(c4)CCNC5=O)c(N)nc3F)cc2)CCO1] | A | D |
| I-352 | 6-(2-amino-6-fluoro-5-(4- ((1R,5S)-3-methyl-3- azabicyclo[3.1.0]hexan- 1-yl)phenyl)pyridin-3-yl)- 8-fluoro-3,4- dihydroisoquinolin- 1(2H)-one | 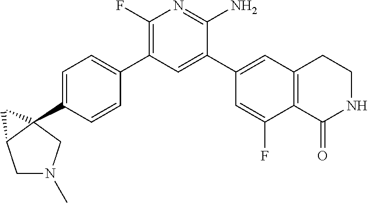 | A | D |
| I-353 | 6-(2-amino-6-fluoro-5-(4- ((1S,5R)-3-methyl-3- azabicyclo[3.1.0]hexan- 1-yl)phenyl)pyridin-3-yl)- 8-fluoro-3,4- dihydroisoquinolin- 1(2H)-one | 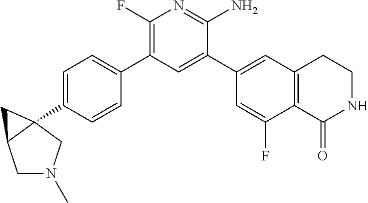 [SMILES: CN1C[C@H]2C[C@@]2(c2ccc(-c3cc(-c4cc(F)c5c(c4)CCNC5=O)c(N)nc3F)cc2)C1] | A | C |
| I-354 | 6-(2-amino-6-fluoro-5-(4- (4-isopropylpiperazin-1- yl)phenyl)pyridin-3-yl)-8- methoxy-3,4- dihydroisoquinolin- 1(2H)-one | 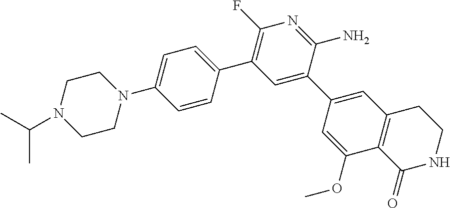 [SMILES: COc1cc(-c2cc(-c3ccc(N4CCN(C(C)C)CC4)cc3)c(F)nc2N)cc2c1C(=O)NCC2] | C | B |
| I-355 | 6-(2-amino-6-fluoro-5-(4- (4-isopropylpiperazin-1- yl)phenyl)pyridin-3-yl)-8- (dimethylamino)-3,4- dihydroisoquinolin- 1(2H)-one | 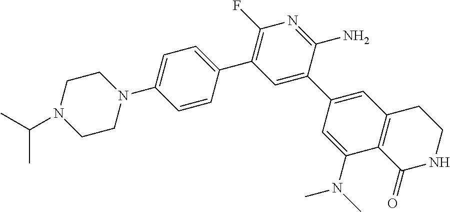 [SMILES: CC(C)N1CCN(c2ccc(-c3cc(-c4cc5c(c(N(C)C)c4)C(=O)NCC5)c(N)nc3F)cc2)CC1] | C | D |
| I-356 | 6-(2-amino-6-fluoro-5-(4- (4-isopropylpiperazin-1- yl)phenyl)pyridin-3-yl)-8- (methylamino)-3,4- dihydroisoquinolin- 1(2H)-one | 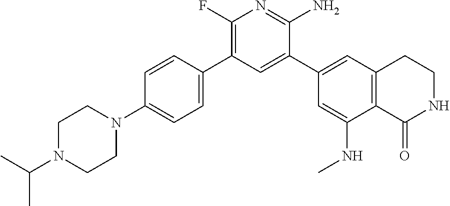 [SMILES: CNc1cc(-c2cc(-c3ccc(N4CCN(C(C)C)CC4)cc3)c(F)nc2N)cc2c1C(=O)NCC2] | B | C |
| I-357 | 8-amino-6-(2-amino-6- fluoro-5-(4-(4- isopropylpiperazin-1- yl)phenyl)pyridin-3-yl)- 3,4-dihydroisoquinolin- 1(2H)-one | 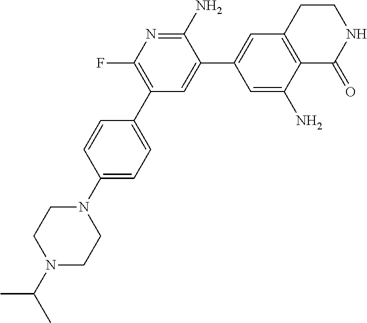 [SMILES: CC(C)N1CCN(c2ccc(-c3cc(-c4cc(N)c5c(c4)CCNC5=O)c(N)nc3F)cc2)CC1] | C | C |
| I-358 | 8-(2-amino-6-fluoro-5-(4- (4-isopropylpiperazin-1- yl)phenyl)pyridin-3-yl)- 3,4- dihydrobenzo[f][1,4]oxaze- pin-5(2H)-one | 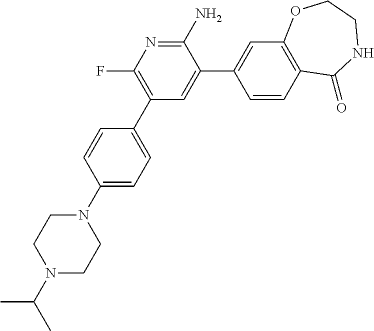 [SMILES: CC(C)N1CCN(c2ccc(-c3cc(-c4ccc5c(c4)OCCNC5=O)c(N)nc3F)cc2)CC1] | A | D |
| I-359 | 6-(2-amino-5-(4-(1,1- dioxidothiomorpholino)phe- nyl)-6-fluoropyridin-3- yl)-8-fluoro-3,4- dihydroisoquinolin- 1(2H)-one | 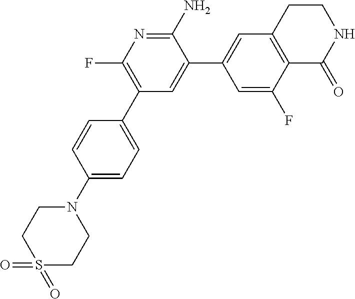 [SMILES: Nc1nc(F)c(-c2ccc(N3CCS(=O)(=O)CC3)cc2)cc1-c1cc(F)c2c(c1)CCNC2=O] | A | D |
| I-360 | 6-(2-amino-6-fluoro-5-(4- (morpholinomethyl)phenyl) pyridin-3-yl)-8-fluoro- 3,4-dihydroisoquinoin- 1(2H)-one | 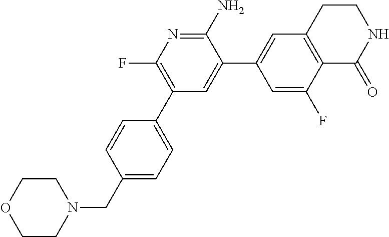 [SMILES: Nc1nc(F)c(-c2ccc(CN3CCOCC3)cc2)cc1-c1cc(F)c2c(c1)CCNC2=O] | A | |
| I-361 | 6-(2-amino-6-fluoro-5-(3- fluoro-4- morpholinophenyl)pyridin- 3-yl)-8-fluoro-3,4- dihydroisoquinolin- 1(2H)-one | 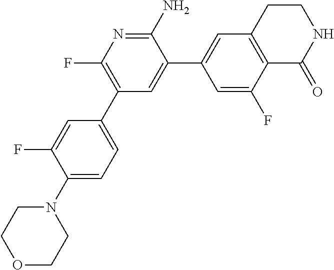 [SMILES: Nc1nc(F)c(-c2ccc(N3CCOCC3)c(F)c2)cc1-c1cc(F)c2c(c1)CCNC2=O] | A | |
| I-362 | 7-(2-amino-6-fluoro-5-(4- (4-isopropylpiperazin-1- yl)phenyl)pyridin-3- yl)quinazolin-4(3H)-one | 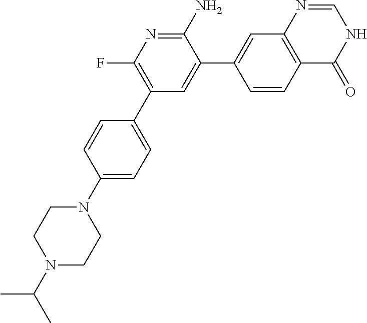 [SMILES: CC(C)N1CCN(c2ccc(-c3cc(-c4ccc5c(=O)[nH]cnc5c4)c(N)nc3F)cc2)CC1] | A | D |
| I-363 | 6-(2-amino-6-fluoro-5-(4- (4-isopropylpiperazin-1- yl)phenyl)pyridin-3- yl)phthalazin-1(2H)-one | 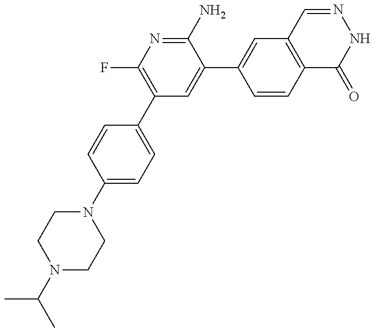 [SMILES: CC(C)N1CCN(c2ccc(-c3cc(-c4ccc5c(=O)[nH]ncc5c4)c(N)nc3F)cc2)CC1] | B | D |
| I-364 | 6-(2-amino-6-fluoro-5-(4- (4-isopropylpiperazin-1- yl)phenyl)pyridin-3- yl)isoquinolin-1(2H)-one | 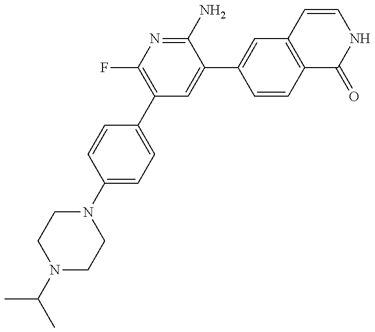 [SMILES: CC(C)N1CCN(c2ccc(-c3cc(-c4ccc5c(=O)[nH]ccc5c4)c(N)nc3F)cc2)CC1] | A | D |
| I-367 | (S)-6-(2-amino-6-fluoro- 5-(3-(pyrrolidin-2-yl)-4- (tetrahydro-2H-pyran-4- yl)phenyl)pyridin-3-yl)- 3,4-dihydroisoquinolin- 1(2H)-one | 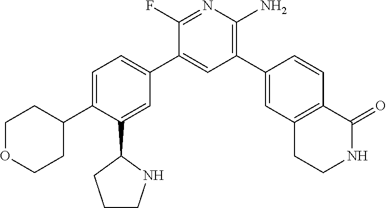 [SMILES: Nc1nc(F)c(-c2ccc(C3CCOCC3)c([C@@H]3CCCN3)c2)cc1-c1ccc2c(c1)CCNC2=O] | A | C |
| I-368 | (S)-6-(2-amino-6-fluoro- 5-(3-(pyrrolidin-2-yl)-4- (tetrahydro-2H-pyran-4- yl)phenyl)pyridin-3-yl)-7- fluoro-3,4- dihydroisoquinolin- 1(2H)-one | 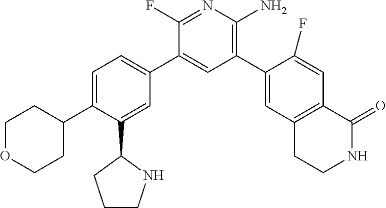 [SMILES: Nc1nc(F)c(-c2ccc(C3CCOCC3)c([C@@H]3CCCN3)c2)cc1-c1cc2c(cc1F)C(=O)NCC2] | A | B |
| I-369 | (S)-6-(2-amino-6-fluoro- 5-(3-(pyrrolidin-2-yl)-4- (tetrahydro-2H-pyran-4- yl)phenyl)pyridin-3-yl)-8- fluoro-3,4- dihydroisoquinolin- 1(2H)-one | 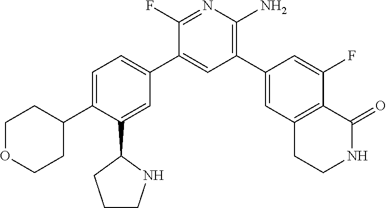 [SMILES: Nc1nc(F)c(-c2ccc(C3CCOCC3)c([C@@H]3CCCN3)c2)cc1-c1cc(F)c2c(c1)CCNC2=O] | A | D |
| I-370 | (S)-6-(2-amino-6-fluoro- 5-(3-(pyrrolidin-2-yl)-4- (tetrahydro-2H-pyran-4- yl)phenyl)pyridin-3- y)isoquinolin-1(2H)-one | 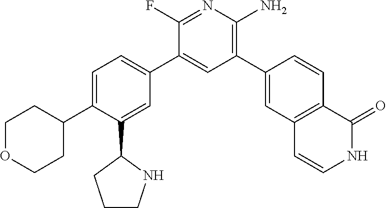 [SMILES: Nc1nc(F)c(-c2ccc(C3CCOCC3)c([C@@H]3CCCN3)c2)cc1-c1ccc2c(=O)[nH]ccc2c1] | A | D |
| I-372 | 6-(2-amino-6-fluoro-5-(4- (4-isopropylpiperazin-1- yl)phenyl)pyridin-3-yl)-8- fluoroisoquinolin-1(2H)- one | 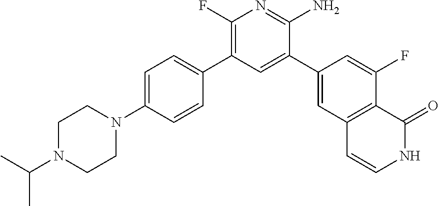 [SMILES: CC(C)N1CCN(c2ccc(-c3cc(-c4cc(F)c5c(=O)[nH]ccc5c4)c(N)nc3F)cc2)CC1] | A | C |
| I-373 | 6-(2-amino-6-fluoro-5-(4- (4-isopropylpiperazin-1- yl)phenyl)pyridin-3-yl)-7- fluoroisoquinolin-1(2H)- one | 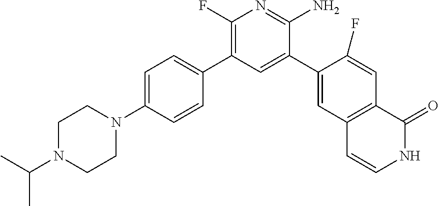 [SMILES: CC(C)N1CCN(c2ccc(-c3cc(-c4cc5cc[nH]c(=O)c5cc4F)c(N)nc3F)cc2)CC1] | A | B |
| I-374 | 6-(2-amino-5-(4-(4- (cyclopropylmethyl)piper- azin-1-yl)phenyl)-6- fluoropyridin-3- yl)isoquinolin-1(2H)-one | 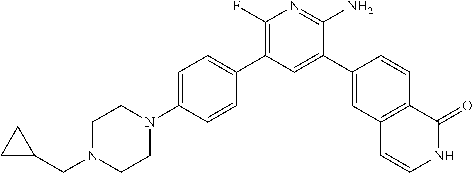 [SMILES: Nc1nc(F)c(-c2ccc(N3CCN(CC4CC4)CC3)cc2)cc1-c1ccc2c(=O)[nH]ccc2c1] | A | D |
| I-375 | 6-(2-amino-5-(4-(4- (cyclopropylmethyl)piper- azin-1-yl)phenyl)-6- fluoropyridin-3-yl)-8- fluoroisoquinolin-1(2H)- one | 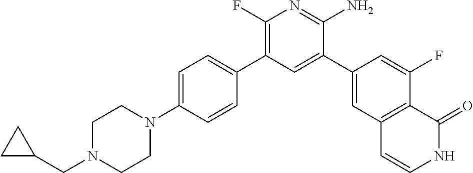 [SMILES: Nc1nc(F)c(-c2ccc(N3CCN(CC4CC4)CC3)cc2)cc1-c1cc(F)c2c(=O)[nH]ccc2c1] | A | D |
| I-376 | 6-(2-amino-5-(4-(4- (cyclopropylmethyl)piper- azin-1-yl)phenyl)-6- fluoropyridin-3-yl)-7- fluoroisoquinolin-1(2H)- one | 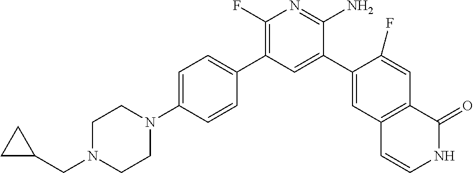 [SMILES: Nc1nc(F)c(-c2ccc(N3CCN(CC4CC4)CC3)cc2)cc1-c1cc2cc[nH]c(=O)c2cc1F] | A | D |
| I-377 | 6-(2-amino-6-fluoro-5-(4- (4-isoporpylpiperazin-1- yl)phenyl)pyridin-3-yl)-3- methylisoquinolin-1(2H)- one | 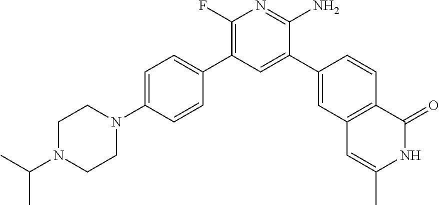 [SMILES: Cc1cc2cc(-c3cc(-c4ccc(N5CCN(C(C)C)CC5)cc4)c(F)nc3N)ccc2c(=O)[nH]1] | A | C |
| I-378 | 6-(2-amino-6-fluoro-5-(4- (4-isopropylpiperazin-1- yl)phenyl)pyridin-3-yl)-4- methylisoquinolin-1(2H)- one | 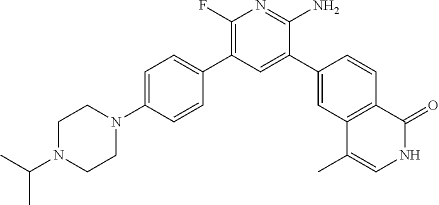 [SMILES: Cc1c[nH]c(=O)c2ccc(-c3cc(-c4ccc(N5CCN(C(C)C)CC5)cc4)c(F)nc3N)cc12] | A | D |
| I-379 | 6-(2-amino-6-fluoro-5-(4- (4-isopropylpiperazin-1- yl)phenyl)pyridin-3-yl)-4- fluoroisoquinolin-1(2H)- one | 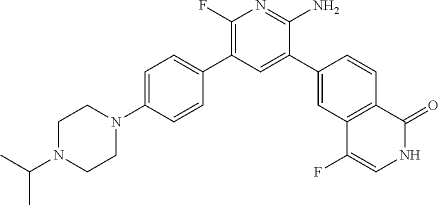 [SMILES: CC(C)N1CCN(c2ccc(-c3cc(-c4ccc5c(=O)[nH]cc(F)c5c4)c(N)nc3F)cc2)CC1] | A | D |
| I-380 | 6-(2-amino-6-fluoro-5-(4- (4-isopropylpiperazin-1- yl)phenyl)pyridin-3-yl)-8- fluoro-3- methylisoquinolin-1(2H)- one | 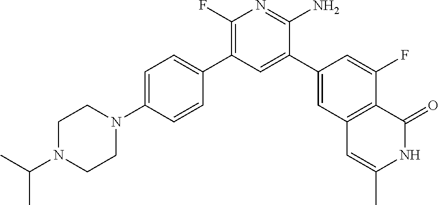 [SMILES: Cc1cc2cc(-c3cc(-c4ccc(N5CCN(C(C)C)CC5)cc4)c(F)nc3N)cc(F)c2c(=O)[nH]1] | A | D |
| I-381 | 6-(2-amino-6-fluoro-5-(4- (4-isopropylpiperazin-1- yl)phenyl)pyridin-3-yl)-4- fluoro-3- methylisoquinolin-1(2H)- one | 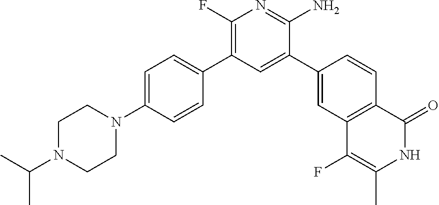 [SMILES: Cc1[nH]c(=O)c2ccc(-c3cc(-c4ccc(N5CCN(C(C)C)CC5)cc4)c(F)nc3N)cc2c1F] | C | D |
| I-384 | 6-(2-amino-5-(4-(4-(2- cyclopropylethyl)piperazin- 1-yl)phenyl)-6- fluoropyridin-3-yl)-8- fluoro-3,4- dihydroisoquinolin- 1(2H)-one | 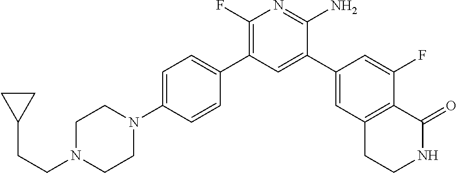 [SMILES: Nc1nc(F)c(-c2ccc(N3CCN(CCC4CC4)CC3)cc2)cc1-c1cc(F)c2c(c1)CCNC2=O] | A | C |
| I-385 | 6-(2-amino-5-(4-(4- (cyclopropylmethyl)piper- azin-1-yl)-3- fluorophenyl)-6- fluoropyridin-3-yl)-8- fluoro-3,4- dihydroisoquinolin- 1(2H)-one | 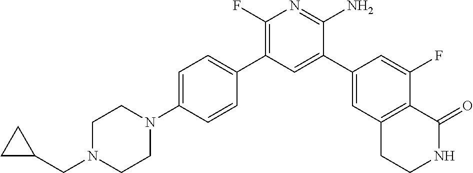 [SMILES: Nc1nc(F)c(-c2ccc(N3CCN(CC4CC4)CC3)cc2)cc1-c1cc(F)c2c(c1)CCNC2=O] | A | D |
| I-386 | 6-(2-amino-6-fluoro-5-(3- fluoro-4-(4- isopropylpiperazin-1- yl)phenyl)pyridin-3-yl)-8- fluoro-3,4- dihydroisoquinolin- 1(2H)-one | 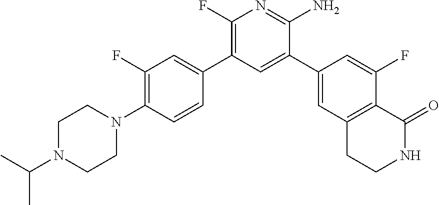 [SMILES: CC(C)N1CCN(c2ccc(-c3cc(-c4cc(F)c5c(c4)CCNC5=O)c(N)nc3F)cc2F)CC1] | A | A |
| I-387 | 6-(2-amino-5-(4-(4-(2- cyclopropylethyl)piperazin- 1-yl)phenyl)-6- fluoropyridin-3-yl)-8- fluoroisoquinolin-1(2H)- one | 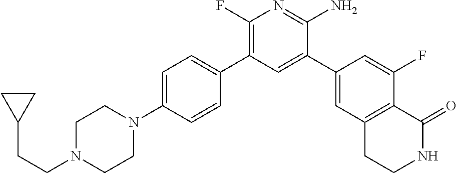 [SMILES: Nc1nc(F)c(-c2ccc(N3CCN(CCC4CC4)CC3)cc2)cc1-c1cc(F)c2c(c1)CCNC2=O] | A | D |
| I-388 | 6-(2-amino-5-(4-(4-(2- cyclopropylethyl)piperazin- 1-yl)phenyl)-6- fluoropyridin-3-yl)-7- fluroisoquinolin-1(2H)- one | 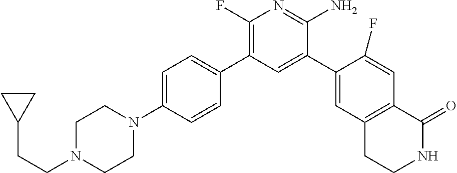 [SMILES: Nc1nc(F)c(-c2ccc(N3CCN(CCC4CC4)CC3)cc2)cc1-c1cc2c(cc1F)C(=O)NCC2] | A | D |
| I-389 | 6-(2-amino-5-(4-(4-(2- cyclopropylethyl)piperazin- 1-yl)phenyl)-6- fluoropyridin-3- yl)isoquinolin-1(2H)-one | 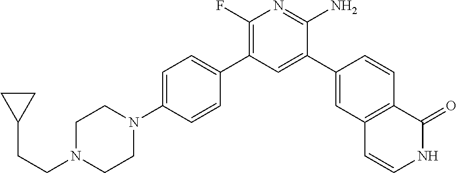 [SMILES: Nc1nc(F)c(-c2ccc(N3CCN(CCC4CC4)CC3)cc2)cc1-c1ccc2c(=O)[nH]ccc2c1] | A | D |
| I-390 | (R)-6-(2-amino-5-(4-(4- (1- cyclopropylethyl)piperazin- 1-yl)phenyl)-6- fluoropyridin-3-yl)-8- fluoro-3,4- dihydroisoquinolin- 1(2H)-one | 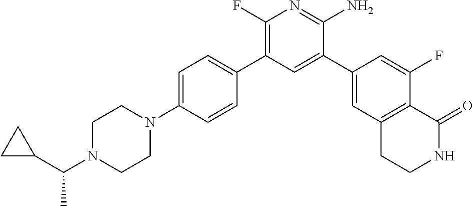 [SMILES: C[C@H](C1CC1)N1CCN(c2ccc(-c3cc(-c4cc(F)c5c(c4)CCNC5=O)c(N)nc3F)cc2)CC1] | A | C |
| I-391 | (R)-6-(2-amino-5-(4-(4- (1- cyclopropylethyl)piperazin- 1-yl)phenyl)-6- fluoropyridin-3-yl)-8- fluoroisoquinolin-1(2H)- one | 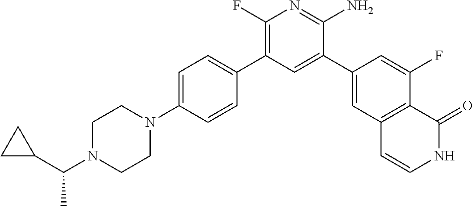 [SMILES: C[C@H](C1CC1)N1CCN(c2ccc(-c3cc(-c4cc(F)c5c(=O)[nH]ccc5c4)c(N)nc3F)cc2)CC1] | A | C |
| I-392 | (S)-6-(2-amino-5-(4-(4- (1- cyclopropylethyl)piperazin- 1-yl)phenyl)-6- fluoropyridin-3-yl)-8- fluoro-3,4- dihydroisoquinolin- 1(2H)-one | 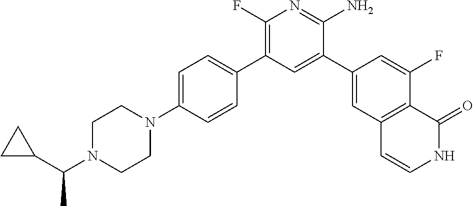 [SMILES: C[C@@H](C1CC1)N1CCN(c2ccc(-c3cc(-c4cc(F)c5c(=O)[nH]ccc5c4)c(N)nc3F)cc2)CC1] | A | C |
| I-393 | (S)-6-(2-amino-5-(4-(4- (1- cyclopropylethyl)piperazin- 1-yl)phenyl)-6- fluoropyridin-3-yl)-8- fluoroisoquinolin-1(2H)- one | 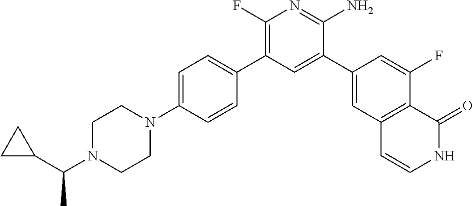 [SMILES: C[C@@H](C1CC1)N1CCN(c2ccc(-c3cc(-c4cc(F)c5c(=O)[nH]ccc5c4)c(N)nc3F)cc2)CC1] | A | C |
| I-394 | 6-(2-amino-5-(4-(4- ethylpiperazin-1- yl)phenyl)-6- fluoropyridin-3-yl)-8- fluoroisoquinolin-1(2H)- one | 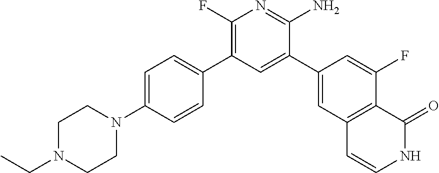 [SMILES: CCN1CCN(c2ccc(-c3cc(-c4cc(F)c5c(=O)[nH]ccc5c4)c(N)nc3F)cc2)CC1] | A | C |
| I-396 | 6-(2-amino-6-fluoro-5-(4- morpholinophenyl)pyridin- 3-yl)-8- fluoroisoquinolin-1(2H)- one | 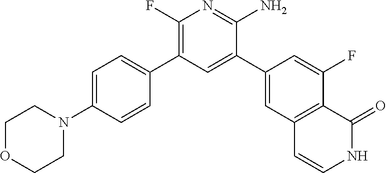 [SMILES: Nc1nc(F)c(-c2ccc(N3CCOCC3)cc2)cc1-c1cc(F)c2c(=O)[nH]ccc2c1] | A | D |
| I-397 | (S)-6-(2-amino-6-fluoro- 5-(4-(2- isopropylmorpholino)phe- nyl)pyridin-3-yl)-3,4- dihydro-2,7-naphthyridin- 1(2H)-one | 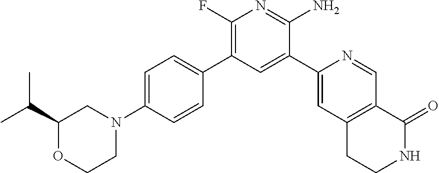 [SMILES: CC(C)[C@H]1CN(c2ccc(-c3cc(-c4cc5c(cn4)C(=O)NCC5)c(N)nc3F)cc2)CCO1] | A | D |
| I-398 | 6-(2-amino-6-fluoro-5-(4- (4-isopropylpiperazin-1- yl)phenyl)pyridin-3-yl)- 3,4-dihydro-2,7- naphthyridin-1(2H)-one | 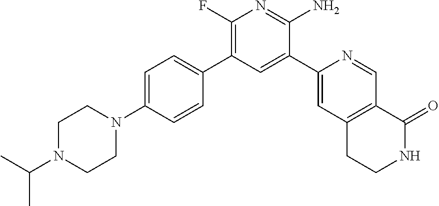 [SMILES: CC(C)N1CCN(c2ccc(-c3cc(-c4cc5c(cn4)C(=O)NCC5)c(N)nc3F)cc2)CC1] | A | C |
| I-399 | 3-(2-amino-6-fluoro-5-(4- morpholinophenyl)pyridin- 3-yl)-1,7-naphthyridin- 8(7H)-one | 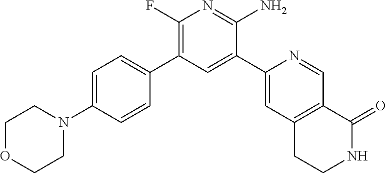 [SMILES: Nc1nc(F)c(-c2ccc(N3CCOCC3)cc2)cc1-c1cc2c(cn1)C(=O)NCC2] | B | D |
| I-400 | 6-(2-amino-5-(4-(4- (cyclopropylmethyl)piper- azin-1-yl)phenyl)-6- fluoropyridin-3-yl)-3,4- dihydro-2,7-naphthyridin- 1(2H)-one | 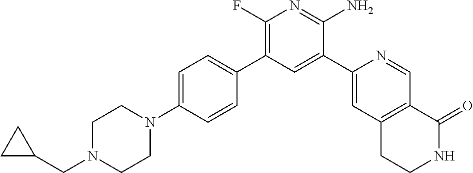 [SMILES: Nc1nc(F)c(-c2ccc(N3CCN(CC4CC4)CC3)cc2)cc1-c1cc2c(cn1)C(=O)NCC2] | A | C |
| I-401 | 6-(2-amino-6-fluoro-5-(4- (4-isopropylpiperazin-1- yl)phenyl)pyridin-3-yl)- 2,7-naphthyridin-1(2H)- one | 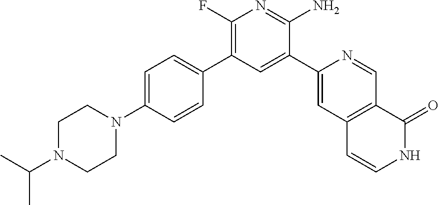 [SMILES: CC(C)N1CCN(c2ccc(-c3cc(-c4cc5cc[nH]c(=O)c5cn4)c(N)nc3F)cc2)CC1] | A | D |
| I-402 | 3-(2-amino-6-fluoro-5-(4- (4-isopropylpiperazin-1- yl)phenyl)pyridin-3-yl)- 1,7-naphthyridin-8(7H)- one | 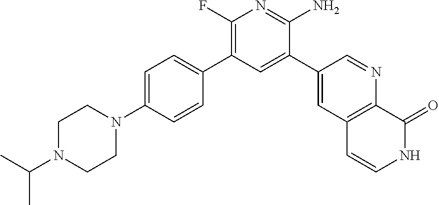 [SMILES: CC(C)N1CCN(c2ccc(-c3cc(-c4cnc5c(=O)[nH]ccc5c4)c(N)nc3F)cc2)CC1] | A | D |
| I-405 | 7-(2-amino-6-fluoro-5-(4- (4-isopropylpiperazin-1- y)phenyl)pyridin-3-yl)-5- fluoroquinazolin-4(3H)- one | 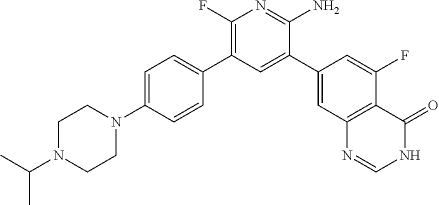 [SMILES: CC(C)N1CCN(c2ccc(-c3cc(-c4cc(F)c5c(=O)[nH]cnc5c4)c(N)nc3F)cc2)CC1] | A | D |
| I-406 | 7-(2-amino-6-fluoro-5-(4- (4-isopropylpiperazin-1- yl)phenyl)pyridin-3-yl)-6- fluoroquinazolin-4(3H)- one | 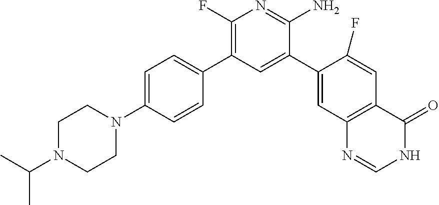 [SMILES: CC(C)N1CCN(c2ccc(-c3cc(-c4cc5nc[nH]c(=O)c5cc4F)c(N)nc3F)cc2)CC1] | A | D |
| I-407 | 7-(2-amino-5-(4-(4- (cyclopropylmethyl)piper- azin-1-yl)phenyl)-6- fluoropyridin-3-yl)-5- fluoroquinazolin-4(3H)- one | 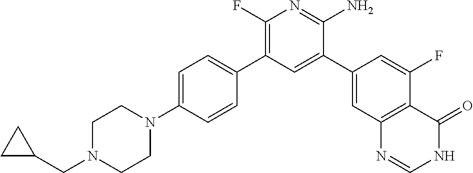 [SMILES: Nc1nc(F)c(-c2ccc(N3CCN(CC4CC4)CC3)cc2)cc1-c1cc(F)c2c(=O)[nH]cnc2c1] | A | D |
| I-408 | 7-(2-amino-5-(4-(4- (cyclopropylmethyl)piper- azin-1-yl)phenyl)-6- fluoropyridin-3-yl)-6- fluoroquinazolin-4(3H)- one | 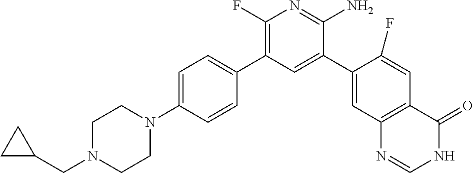 [SMILES: Nc1nc(F)c(-c2ccc(N3CCN(CC4CC4)CC3)cc2)cc1-c1cc2nc[nH]c(=O)c2cc1F] | A | D |
| I-409 | 7-(2-amino-6-fluoro-5-(4- (4-isopropylpiperazin-1- yl)phenyl)pyridin-3-yl)-2- methylquinazolin-4(3H)- one | 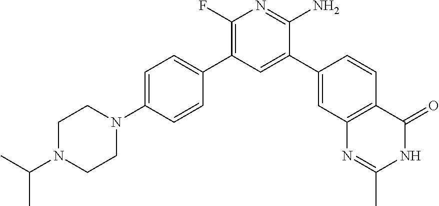 [SMILES: Cc1nc2cc(-c3cc(-c4ccc(N5CCN(C(C)C)CC5)cc4)c(F)nc3N)ccc2c(=O)[nH]1] | A | D |
| I-410 | 7-(2-amino-6-fluoro-5-(4- (4-isopropylpiperazin-1- yl)phenyl)pyridin-3-yl)-2- ((methylamino)methyl)quin- azolin-4(3H)-one | 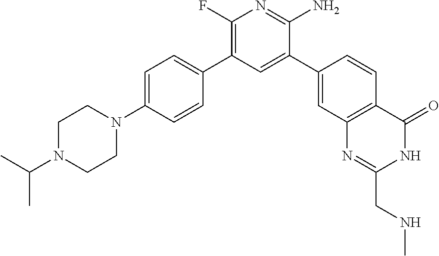 [SMILES: CNCc1nc2cc(-c3cc(-c4ccc(N5CCN(C(C)C)CC5)cc4)c(F)nc3N)ccc2c(=O)[nH]1] | B | D |
| I-411 | 7-(2-amino-5-(4-(4- (cyclopropylmethyl)piper- azin-1-yl)phenyl)-6- fluoropyridin-3-yl)-2- methylquinazolin-4(3H)- one | 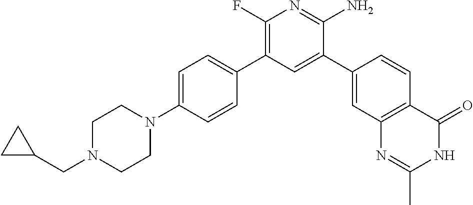 [SMILES: Cc1nc2cc(-c3cc(-c4ccc(N5CCN(CC6CC6)CC5)cc4)c(F)nc3N)ccc2c(=O)[nH]1] | B | D |
| I-412 | 7-(2-amino-6-fluoro-5-(4- (4-isoopropylpiperazin-1- yl)phenyl)pyridin-3-yl)-2- cyclopropylquinazolin- 4(3H)-one | 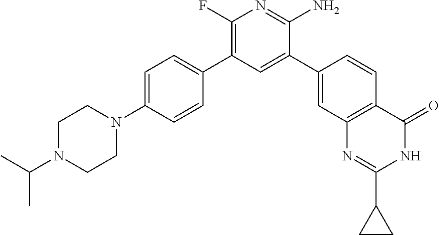 [SMILES: CC(C)N1CCN(c2ccc(-c3cc(-c4ccc5c(=O)[nH]c(C6CC6)nc5c4)c(N)nc3F)cc2)CC1] | C | |
| I-413 | 7-(2-amino-6-fluoro-5-(4- (4-isopropylpiperazin-1- yl)phenyl)pyridin-3-yl)-2- cyclobutylquinazolin- 4(3H)-one | 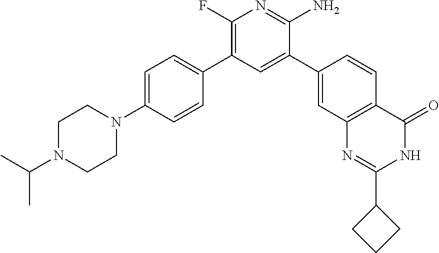 [SMILES: CC(C)N1CCN(c2ccc(-c3cc(-c4ccc5c(=O)[nH]c(C6CCC6)nc5c4)c(N)nc3F)cc2)CC1] | C | |
| I-414 | 7-(2-amino-6-fluoro-5-(4- (4-isopropylpiperazin-1- yl)phenyl)pyridin-3-yl)-5- fluoro-2- methylquinazolin-4(3H)- one | 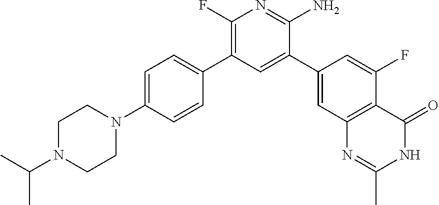 [SMILES: Cc1nc2cc(-c3cc(-c4ccc(N5CCN(C(C)C)CC5)cc4)c(F)nc3N)cc(F)c2c(=O)[nH]1] | A | D |
| I-416 | 7-(2-amino-5-(4-(4- (cyclopropylmethyl)piper- azin-1-yl)phenyl)-6- fluoropyridin-3-yl)-5- fluoro-2- methylquinazolin-4(3H)- one | 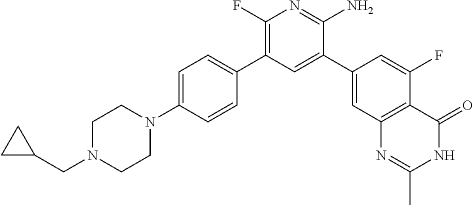 [SMILES: Cc1nc2cc(-c3cc(-c4ccc(N5CCN(CC6CC6)CC5)cc4)c(F)nc3N)cc(F)c2c(=O)[nH]1] | A | D |
| I-417 | 7-(2-amino-5-(4-(4-(2- cyclopropylethyl)piperazin- 1-yl)phenyl)-6- fluoropyridin-3-yl)-5- fluoro-2- methylquinazolin-4(3H)- one | 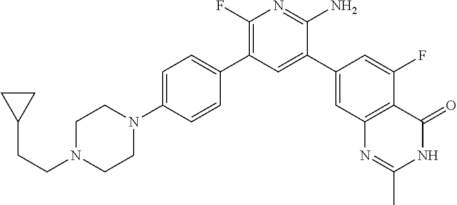 [SMILES: Cc1nc2cc(-c3cc(-c4ccc(N5CCN(CCC6CC6)CC5)cc4)c(F)nc3N)cc(F)c2c(=O)[nH]1] | A | D |
| I-418 | 7-(2-amino-6-fluoro-5-(4- (4-isopropylpiperazin-1- yl)phenyl)pyridin-3-yl)-2- chloroquinazolin-4(3H)- one | 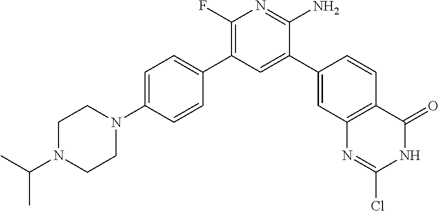 [SMILES: CC(C)N1CCN(c2ccc(-c3cc(-c4ccc5c(=O)[nH]c(Cl)nc5c4)c(N)nc3F)cc2)CC1] | C | |
| I-419 | 7-(2-amino-6-fluoro-5-(4- (4-isopropylpiperazin-1- yl)phenyl)pyridin-3-yl)-2- methoxyquinazolin- 4(3H)-one | 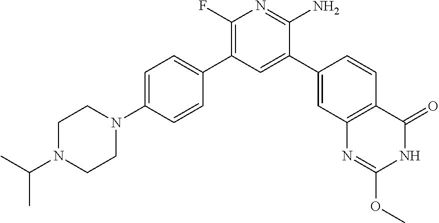 [SMILES: COc1nc2cc(-c3cc(-c4ccc(N5CCN(C(C)C)CC5)cc4)c(F)nc3N)ccc2c(=O)[nH]1] | B | |
| I-420 | 7-(2-amino-6-fluoro-5-(4- (4-isopropylpiperazin-1- yl)phenyl)pyridin-3-yl)-2- (dimethylamino)quinazolin- 4(3H)-one | 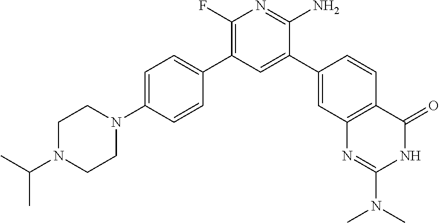 [SMILES: CC(C)N1CCN(c2ccc(-c3cc(-c4ccc5c(=O)[nH]c(N(C)C)nc5c4)c(N)nc3F)cc2)CC1] | C | |
| I-421 | 7-(2-amino-6-fluoro-5-(4- (4-isopropylpiperazin-1- yl)phenyl)pyridin-3-yl)-2- (methylamino)quinazolin- 4(3H)-one | 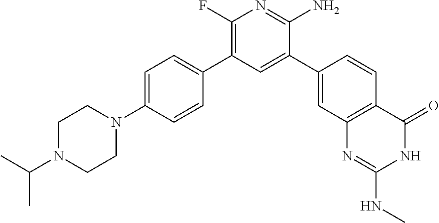 [SMILES: CNc1nc2cc(-c3cc(-c4ccc(N5CCN(C(C)C)CC5)cc4)c(F)nc3N)ccc2c(=O)[nH]1] | B | |
| I-422 | 2-amino-7-(2-amino-6- fluoro-5-(4-(4- isopropylpiperazin-1- yl)phenyl)pyridin-3- yl)quinazolin-4(3H)-one | 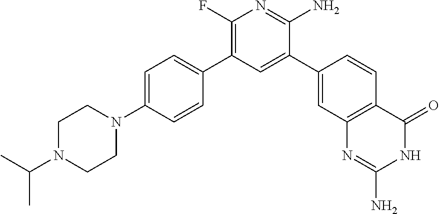 [SMILES: CC(C)N1CCN(c2ccc(-c3cc(-c4ccc5c(=O)[nH]c(N)nc5c4)c(N)nc3F)cc2)CC1] | A | |
| I-424 | 7-(2-amino-6-fluoro-5-(4- (4-isopropylpiperazin-1- yl)phenyl)pyridin-3-yl)- 2,3-dihydro-4H- benzo[e][1,3]thiazin-4- one 1,1-dioxide | 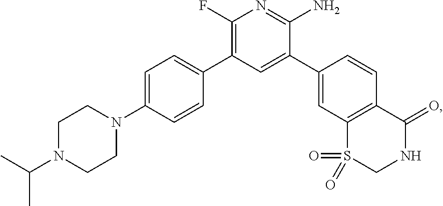 [SMILES: CC(C)N1CCN(c2ccc(-c3cc(-c4ccc5c(c4)S(=O)(=O)CNC5=O)c(N)nc3F)cc2)CC1] | B | |
| | | and | | |
| I-426 | 6-(2-amino-5-(3- ((dimethylamino)methyl)- 4-(tetrahydro-2H-pyran- 4-yl)phenyl)-6- fluoropyridin-3-yl)-4- methylisoquinolin-1(2H)- one | 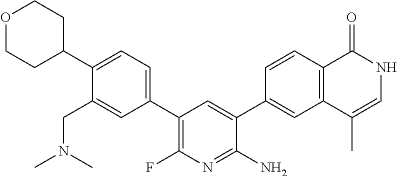 [SMILES: Cc1c[nH]c(=O)c2ccc(-c3cc(-c4ccc(C5CCOCC5)c(CN(C)C)c4)c(F)nc3N)cc12] | A | D |
| I-427 | 6-(6′-amino-6- ((dimethylamino)methyl)- 2′-fluoro-5-morpholino- [2,3′-bipyridin]-5′-yl)-7- fluoro-3,4- dihydroisoquinolin- 1(2H)-one | 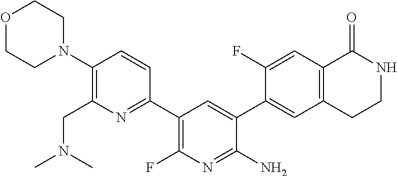 [SMILES: CN(C)Cc1nc(-c2cc(-c3cc4c(cc3F)C(=O)NCC4)c(N)nc2F)ccc1N1CCOCC1] | A | D |
| I-428 | 6-(2-amino-5-(3- ((ethyl(methyl)amio)meth- yl)-4-(tetrahydro-2H- pyran-4-yl)phenyl)-6- fluoropyridin-3-yl)-3,4- dihydroisoquinolin- 1(2H)-one | 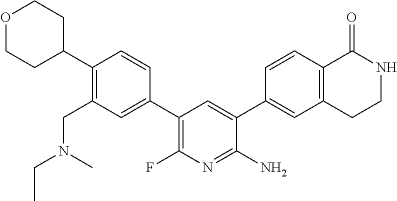 [SMILES: CCN(C)Cc1cc(-c2cc(-c3ccc4c(c3)CCNC4=O)c(N)nc2F)ccc1C1CCOCC1] | B | |
| I-429 | 6-(2-amino-5-(3- ((dimethylamino)methyl)- 4-(tetrahydro-2H-pyran- 4-yl)phenyl)-6- fluoropyridin-3-yl)-7- fluoro-3,4- dihydroisoquinolin- 1(2H)-one | 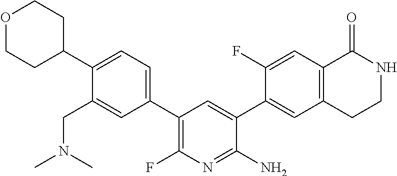 [SMILES: CN(C)Cc1cc(-c2cc(-c3cc4c(cc3F)C(=O)NCC4)c(N)nc2F)ccc1C1CCOCC1] | A | D |
| I-430 | 6-(2-amino-6-fluoro-5-(4- (2-methyl-2,7- diazaspiro[3.5]nonan-7- yl)phenyl)pyridin-3-yl)- 3,4-dihydroisoquinolin- 1(2H)-one | 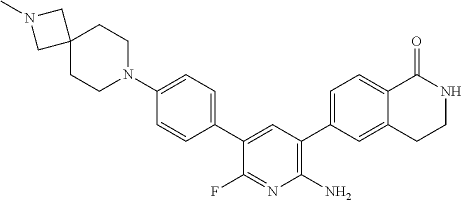 [SMILES: CN1CC2(CCN(c3ccc(-c4cc(-c5ccc6c(c5)CCNC6=O)c(N)nc4F)cc3)CC2)C1] | A | |
| I-431 | 6-(2-amino-6-fluoro-5-(5- methyl-1,2,3,4- tetrahydroisoquinolin-7- yl)pyridin-3-yl)-3,4- dihydroisoquinolin- 1(2H)-one | 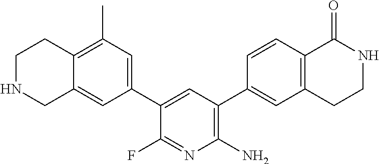 [SMILES: Cc1cc(-c2cc(-c3ccc4c(c3)CCNC4=O)c(N)nc2F)cc2c1CCNC2] | A | D |
| I-432 | 6-(2-amino-5-(3- ((dimethylamino)methyl)- 4-morpholinophenyl)-6- fluoropyridin-3-yl)-3,4- dihydroisoquinolin- 1(2H)-one | 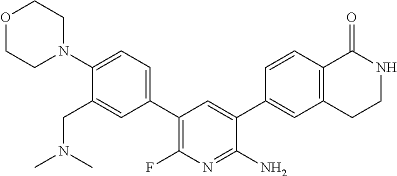 [SMILES: CN(C)Cc1cc(-c2cc(-c3ccc4c(c3)CCNC4=O)c(N)nc2F)ccc1N1CCOCC1] | A | D |
| I-433 | 6-(2-amino-5-(3- ((dimethylamino)methyl)- 4-morpholinophenyl)-6- fluoropyridin-3-yl)-7- fluoro-3,4- dihydroisoquinolin- 1(2H)-one | 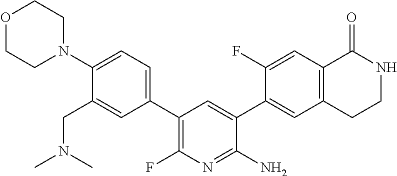 [SMILES: CN(C)Cc1cc(-c2cc(-c3cc4c(cc3F)C(=O)NCC4)c(N)nc2F)ccc1N1CCOCC1] | A | |
| I-434 | 6-(2-amino-5-(2,5- dimethyl-1,2,3,4- tetrahydroisoquinolin-7- yl)-6-fluoropyridin-3-yl)- 3,4-dihydroisoquinolin- 1(2H)-one | 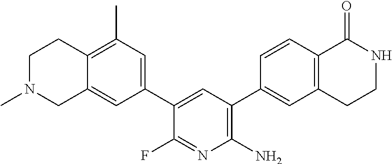 [SMILES: Cc1cc(-c2cc(-c3ccc4c(c3)CCNC4=O)c(N)nc2F)cc2c1CCN(C)C2] | A | D |
| I-435 | 7-(2-amino-6-fluoro-5-(4- (4-isopropylpiperazin-1- yl)phenyl)pyridin-3-yl)-2- (trifluoromethyl)quinazolin- 4(3H)-one | 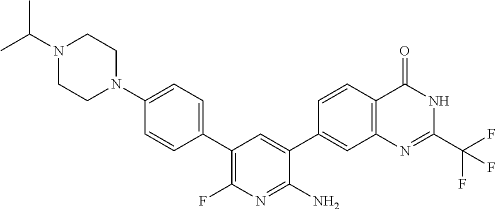 [SMILES: CC(C)N1CCN(c2ccc(-c3cc(-c4ccc5c(=O)[nH]c(C(F)(F)F)nc5c4)c(N)nc3F)cc2)CC1] | C | D |
| I-436 | 6-(2-amino-5-(3- ((dimethylamino)methyl)- 4-(tetrahydro-2H-pyran- 4-yl)phenyl)-6- fluoropyridin-3-yl)-8- fluoro-3,4- dihydroisoquinolin- 1(2H)-one | 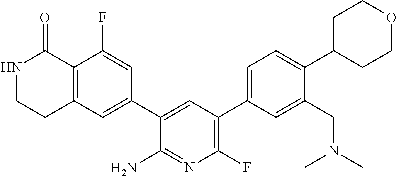 [SMILES: CN(C)Cc1cc(-c2cc(-c3cc(F)c4c(c3)CCNC4=O)c(N)nc2F)ccc1C1CCOCC1] | A | D |
| I-437 | 7-(2-amino-5-(3- ((dimethylamino)methyl)- 4-(tetrahydro-2H-pyran- 4-yl)phenyl)-6- fluoropyridin-3-yl)-2- methylquinazolin-4(3H)- one | 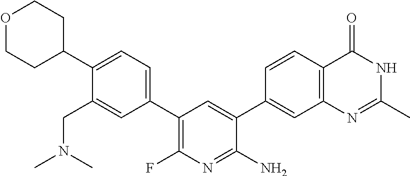 [SMILES: Cc1nc2cc(-c3cc(-c4ccc(C5CCOCC5)c(CN(C)C)c4)c(F)nc3N)ccc2c(=O)[nH]1] | B | D |
| I-438 | 6-(2-amino-6-fluoro-5-(6- methoxy-2-methyl- 1,2,3,4- tetrahydroisoquinolin-7- yl)pyridin-3-yl)-3,4- dihydroisoquinolin- 1(2H)-one | 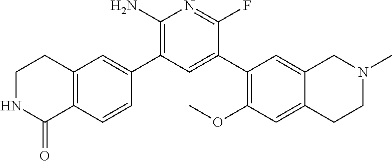 [SMILES: COc1cc2c(cc1-c1cc(-c3ccc4c(c3)CCNC4=O)c(N)nc1F)CN(C)CC2] | B | |
| I-439 | 7-(2-amino-6-fluoro-5-(4- (4-isopropylpiperazin-1- yl)phenyl)pyridin-3-yl)-6- fluoro-2- methylquinazolin-4(3H)- one | 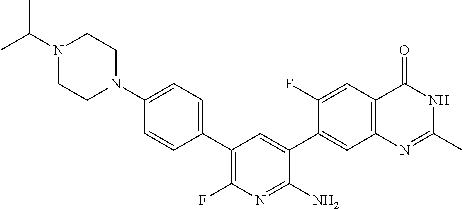 [SMILES: Cc1nc2cc(-c3cc(-c4ccc(N5CCN(C(C)C)CC5)cc4)c(F)nc3N)c(F)cc2c(=O)[nH]1] | B | D |
| I-440 | 6-(2-amino-5-(4-(4-(4,4- difluorobutyl)piperazin-1- yl)phenyl)-6- fluoropyridin-3-yl)-8- fluoro-3,4- dihydroisoquinolin- 1(2H)-one | 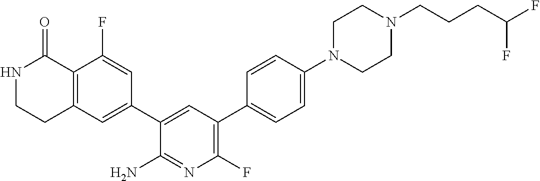 [SMILES: Nc1nc(F)c(-c2ccc(N3CCN(CCCC(F)F)CC3)cc2)cc1-c1cc(F)c2c(c1)CCNC2=O] | A | D |
| I-441 | 6-(2-amino-5-(4-(4-(4,4- difluorobutyl)piperazin-1- yl)phenyl)-6- fluoropyridin-3-yl)-7- fluoro-3,4- dihydroisoquinolin- 1(2H)-one | 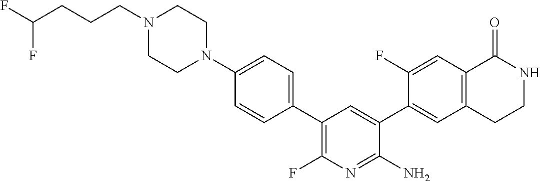 [SMILES: Nc1nc(F)c(-c2ccc(N3CCN(CCCC(F)F)CC3)cc2)cc1-c1cc2c(cc1F)C(=O)NCC2] | Q | D |
| I-442 | tert-butyl 4-(4-(6-amino- 2-fluoro-5-(1-oxo- 1,2,3,4- tetrahydroisoquinolin-6- yl)pyridin-3-yl)-2- ((dimethylamino)methyl) phenyl)piperazine-1- carboxylate | 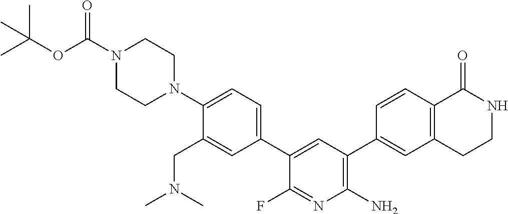 [SMILES: CN(C)Cc1cc(-c2cc(-c3ccc4c(c3)CCNC4=O)c(N)nc2F)ccc1N1CCN(C(=O)OC(C)(C)C)CC1] | A | |
| I-443 | (S)-6-(2-amino-6-fluoro- 5-(3-(1-methylpyrrolidin- 2-yl)phenyl)pyridin-3-yl)- 3,4-dihydroisoquinolin- 1(2H)-one | 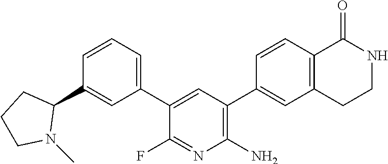 [SMILES: CN1CCC[C@H]1c1cccc(-c2cc(-c3ccc4c(c3)CCNC4=O)c(N)nc2F)c1] | C | |
| I-444 | (S)-6-(2-amino-6-fluoro- 5-(3-(1-methylpyrrolidin- 2-yl)phenyl)pyridin-3-yl)- 7-fluoro-3,4- dihydroisoquinolin- 1(2H)-one | 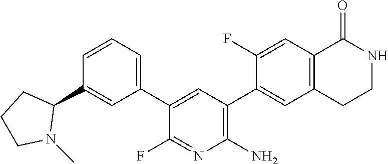 [SMILES: CN1CCC[C@H]1c1cccc(-c2cc(-c3cc4c(cc3F)C(=O)NCC4)c(N)nc2F)c1] | B | |
| I-445 | 6-(2-amino-5-(4-(4-(4,4- difluorobutyl)piperazin-1- yl)-3- ((dimethylamino)methyl) phenyl)-6-fluoropyridin- 3-yl)-3,4- dihydroisoquinolin- 1(2H)-one | 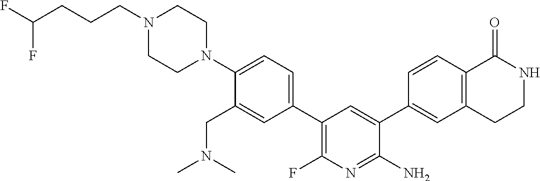 [SMILES: CN(C)Cc1cc(-c2cc(-c3ccc4c(c3)CCNC4=O)c(N)nc2F)ccc1N1CCN(CCCC(F)F)CC1] | A | D |
| I-446 | 6-(2-amino-5-(2-(4,4- difluorobutyl)-5-methyl- 1,2,3,4- tetrahydroisoquinolin-7- yl)-6-fluoropyridin-3-yl)- 3,4-dihydroisoquinolin- 1(2H)-one | 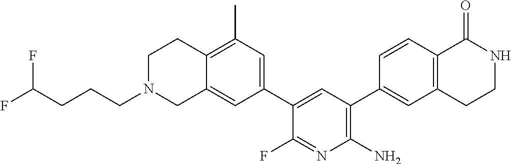 [SMILES: Cc1cc(-c2cc(-c3ccc4c(c3)CCNC4=O)c(N)nc2F)cc2c1CCN(CCCC(F)F)C2] | A | D |
| I-447 | 6-(2-amino-5-(4-(4-(4,4- difluorobutyl)piperazin-1- yl)-3- ((dimethylamino)methyl) phenyl)-6-fluoropyridin- 3-yl)-7-fluroo-3,4- dihydroisoquinolin- 1(2H)-one | 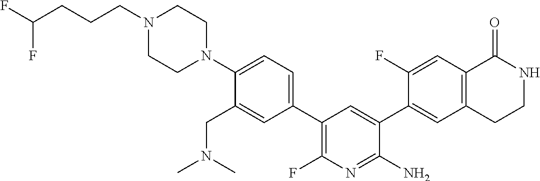 [SMILES: CN(C)Cc1cc(-c2cc(-c3cc4c(cc3F)C(=O)NCC4)c(N)nc2F)ccc1N1CCN(CCCC(F)F)CC1] | A | |
| I-448 | 6-(2-amino-6-fluoro-5-(2- (2-methoxyethyl)-5- methyl-1,2,3,4- tetrahydroisoquinolin-7- yl)pyridin-3-yl)-3,4- dihydroisoquinolin- 1(2H)-one | 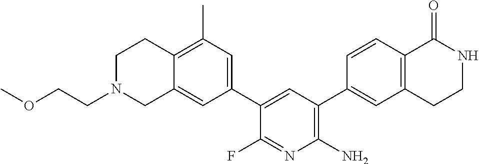 [SMILES: COCCN1CCc2c(C)cc(-c3cc(-c4ccc5c(c4)CCNC5=O)c(N)nc3F)cc2C1] | A | |
| I-449 | 6-(2-amino-5-(4-(2- cyclobutyl-2,7- diazaspiro[3.5]nonan-7- yl)phenyl)-6- fluoropyridin-3-yl)-7- fluoro-3,4- dihydroisoquinolin- 1(2H)-one | 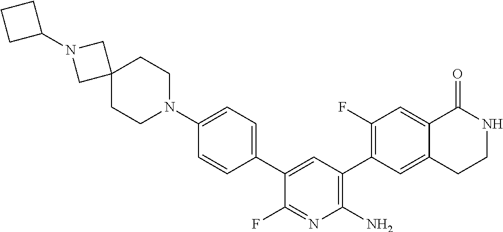 [SMILES: Nc1nc(F)c(-c2ccc(N3CCC4(CC3)CN(C3CCC3)C4)cc2)cc1-c1cc2c(cc1F)C(=O)NCC2] | A | D |
| I-450 | 6-(2-amino-6-fluoro-5-(4- (4-isopropylpiperazin-1- yl)phenyl)pyridin-3-yl)- 4,4-difluoro-3-methoxy- 3-methyl-3,4- dihydroisoquinolin- 1(2H)-one | 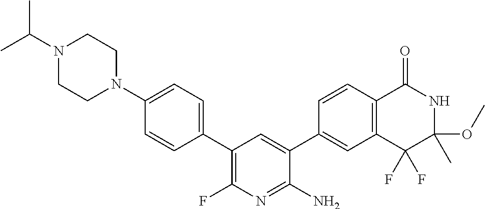 [SMILES: COC1(C)NC(=O)c2ccc(-c3cc(-c4ccc(N5CCN(C(C)C)CC5)cc4)c(F)nc3N)cc2C1(F)F] | B | |
| I-451 | 6-(2-amino-5-(4-(4- cyclopentylpiperazin-1- yl)-3- ((dimethylamino)methyl) phenyl)-6-fluoropyridin- 3-yl)-3,4- dihydroisoquinolin- 1(2H)-one | 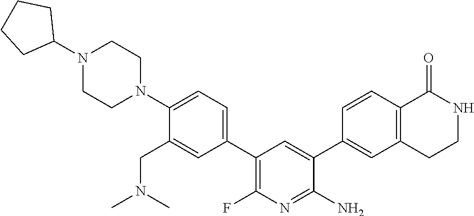 [SMILES: CN(C)Cc1cc(-c2cc(-c3ccc4c(c3)CCNC4=O)c(N)nc2F)ccc1N1CCN(C2CCCC2)CC1] | A | D |
| I-452 | 6-(2-amino-5-(4-(4- cyclopentylpiperazin-1- yl)-3- ((dimethylamino)methyl) phenyl)-6-fluoropyridin- 3-yl)-7-fluoro-3,4- dihydroisoquinolin- 1(2H)-one | 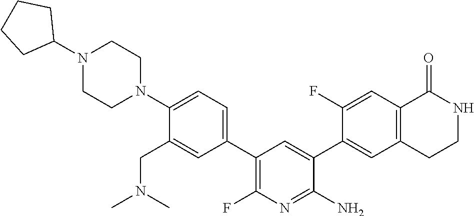 [SMILES: CN(C)Cc1cc(-c2cc(-c3cc4c(cc3F)C(=O)NCC4)c(N)nc2F)ccc1N1CCN(C2CCCC2)CC1] | A | |
| I-453 | 6-(2-amino-5-(4-(4-(4,4- difluorobutyl)piperazin-1- yl)-3- ((dimethylamino)methyl) phenyl)-6-fluoropyridin- 3-yl)isoquinolin-1(2H)- one | 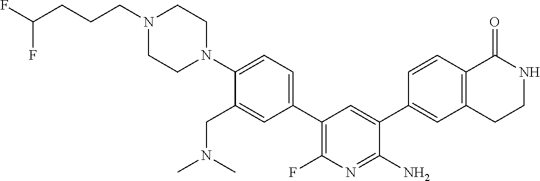 [SMILES: CN(C)Cc1cc(-c2cc(-c3ccc4c(c3)CCNC4=O)c(N)nc2F)ccc1N1CCN(CCCC(F)F)CC1] | A | |
| I-454 | 6-(2-amino-5-(4-(4- (cyclopropylmethyl)piper- azin-1-yl)phenyl)-6- fluoropyridin-3-yl)-8- fluoro-3- methylisoquinolin-1(2H)- one | 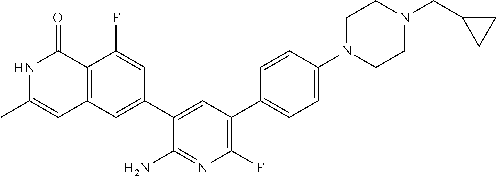 [SMILES: Cc1cc2cc(-c3cc(-c4ccc(N5CCN(CC6CC6)CC5)cc4)c(F)nc3N)cc(F)c2c(=O)[nH]1] | A | D |
| I-455 | 6-(2-amino-5-(4-(4- (cyclopropylmethyl)piper- azin-1-yl)phenyl)-6- fluoropyridin-3-yl)-4- fluoroisoquinolin-1(2H)- one | 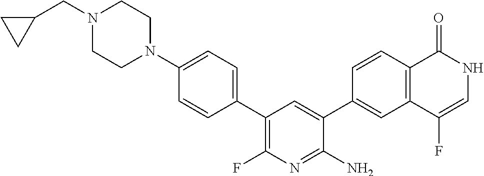 [SMILES: Nc1nc(F)c(-c2ccc(N3CCN(CC4CC4)CC3)cc2)cc1-c1ccc2c(=O)[nH]cc(F)c2c1] | A | D |
| I-456 | 6-(2-amion-5-(4-(4- (cyclopropylmethyl)piper- azin-1-yl)phenyl)-6- fluoropyridin-3-yl)-4- fluoro-3- methylisoquinolin-1(2H)- one | 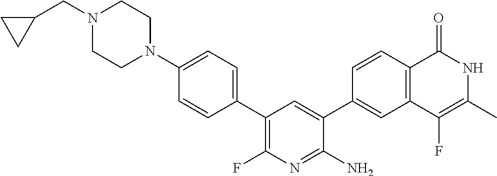 [SMILES: Cc1[nH]c(=O)c2ccc(-c3cc(-c4ccc(N5CCN(CC6CC6)CC5)cc4)c(F)nc3N)cc2c1F] | A | |
| I-457 | 6-(2-amino-5-(4-(4- (cyclopropylmethyl)piper- azin-1-yl)phenyl)-6- fluoropyridin-3-yl)-4- methylisoquinolin-1(2H)- one | 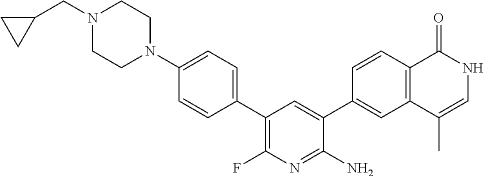 [SMILES: Cc1c[nH]c(=O)c2ccc(-c3cc(-c4ccc(N5CCN(CC6CC6)CC5)cc4)c(F)nc3N)cc12] | A | D |
| I-458 | 2-(2-amino-6-fluoro-5-(4- (4-isopropylpiperazin-1- yl)phenyl)pyridin-3-yl)- 7,8-dihydropyrido[4,3- d]pyrimidin-5(6H)-one | 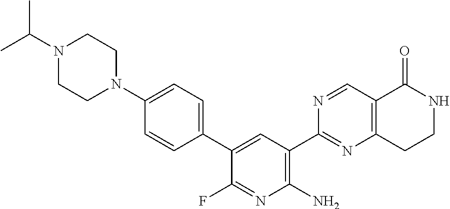 [SMILES: CC(C)N1CCN(c2ccc(-c3cc(-c4ncc5c(n4)CCNC5=O)c(N)nc3F)cc2)CC1] | A | |
| I-459 | 6-(2-amino-5-(3- ((dimethylamino)methyl)- 4-methoxyphenyl)-6- fluoropyridin-3-yl)-3,4- dihydroisoquinolin- 1(2H)-one | 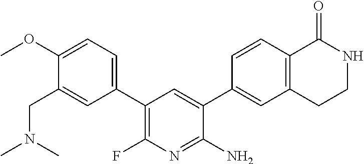 [SMILES: COc1ccc(-c2cc(-c3ccc4c(c3)CCNC4=O)c(N)nc2F)cc1CN(C)C] | B | |
| I-460 | 6-(2-amino-5-(3- ((dimethylamino)methyl)- 4-methoxyphenyl)-6- fluoropyridin-3-yl)-7- fluoro-3,4- dihydroisoquinolin- 1(2H)-one | 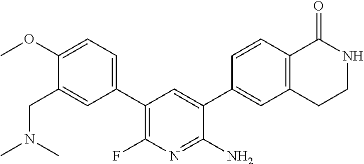 [SMILES: COc1ccc(-c2cc(-c3ccc4c(c3)CCNC4=O)c(N)nc2F)cc1CN(C)C] | B | |
| I-461 | 6-(2-amino-5-(4-(4- cyclobutylpiperazin-1-yl)- 3- ((dimethylamino)methyl) phenyl)-6-fluoropyridin- 3-yl)-3,4- dihydroisoquinolin- 1(2H)-one | 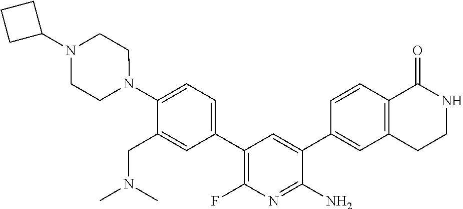 [SMILES: CN(C)Cc1cc(-c2cc(-c3ccc4c(c3)CCNC4=O)c(N)nc2F)ccc1N1CCN(C2CCC2)CC1] | A | |
| I-462 | 6-(2-amino-5-(4-(4- cyclobutylpiperazin-1-yl)- 3- ((dimethylamino)methyl) phenyl)-6-fluoropyridin- 3-yl)-7-fluoro-3,4- dihydroisoquinolin- 1(2H)-one | 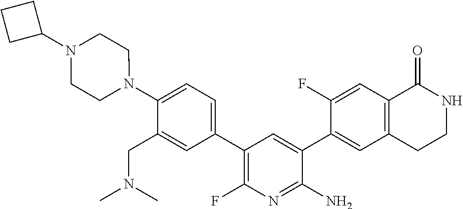 [SMILES: CN(C)Cc1cc(-c2cc(-c3cc4c(cc3F)C(=O)NCC4)c(N)nc2F)ccc1N1CCN(C2CCC2)CC1] | A | |
| I-463 | 6-(2-amino-6-fluoro-5-(2- methyl-1,2,3,4- tetrahydroisoquinolin-5- yl)pyridin-3-yl)-3,4- dihydroisoquinolin- 1(2H)-one | 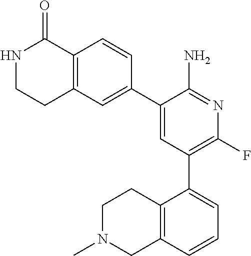 [SMILES: CN1CCc2c(cccc2-c2cc(-c3ccc4c(c3)CCNC4=O)c(N)nc2F)C1] | D | |
| I-464 | 6-(2-amino-6-fluoro-5-(2- methyl-1,2,3,4- tetrahydroisoquinolin-5- yl)pyridin-3-yl)-7-fluoro- 3,4-dihydroisoquinolin- 1(2H)-one | 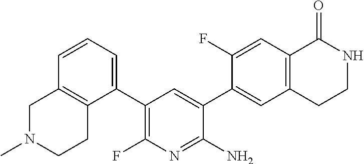 [SMILES: CN1CCc2c(cccc2-c2cc(-c3cc4c(cc3F)C(=O)NCC4)c(N)nc2F)C1] | D | |
| I-465 | 3-amino-7-(2-amino-6- fluoro-5-(4-(4- isopropylpiperazin-1- yl)phenyl)pyridin-3-yl)-2- methylquinazolin-4(3H)- one | 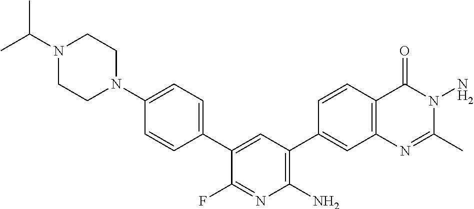 [SMILES: Cc1nc2cc(-c3cc(-c4ccc(N5CCN(C(C)C)CC5)cc4)c(F)nc3N)ccc2c(=O)n1N] | C | |
| I-466 | 6-(2-amino-6-fluoro-5-(4- (4-isopropylpiperazin-1- yl)phenyl)pyridin-3-yl)-4- chloroisoquinolin-1(2H)- one | 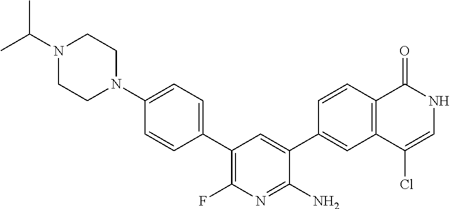 [SMILES: CC(C)N1CCN(c2ccc(-c3cc(-c4ccc5c(=O)[nH]cc(Cl)c5c4)c(N)nc3F)cc2)CC1] | B | |
| I-467 | 7-(2-amino-5-(4-(4-(4,4- difluorobutyl)piperazin-1- yl)phenyl)-6- fluoropyridin-3-yl)-5- fluoro-2- methylquinazolin-4(3H)- one | 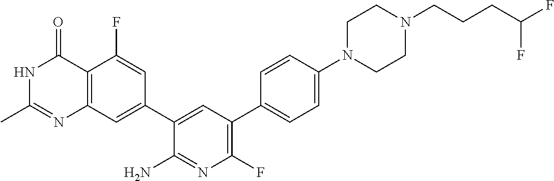 [SMILES: Cc1nc2cc(-c3cc(-c4ccc(N5CCN(CCCC(F)F)CC5)cc4)c(F)nc3N)cc(F)c2c(=O)[nH]1] | A | |
| I-468 | 7-(2-amino-6-fluoro-5-(4- ((1S,5R)-3-methyl-3- azabicyclo[3.1.0]hexan- 1-yl)phenyl)pyridin-3-yl)- 2-methylquinazolin- 4(3H)-one | 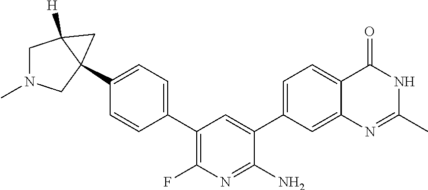 | B | |
| I-469 | 7-(2-amino-6-fluoro-5-(4- ((1S,5R)-3-methyl-3- azabicyclo[3.1.0]hexan- 1-yl)phenyl)pyridin-3-yl)- 5-fluoro-2- methylquinazolin-4(3H)- one | 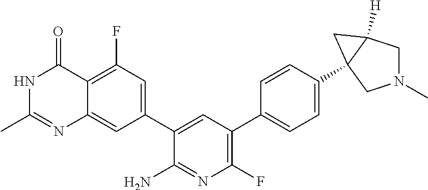 | B | |
| I-470 | 6-(2-amino-6-fluoro-5-(4- ((1S,5R)-3-methyl-3- azabicyclo[3.1.0]hexan- 1-yl)phenyl)pyridin-3-yl)- 4-fluoroisoquinolin- 1(2H)-one | 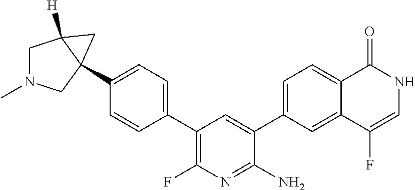 [SMILES: CN1C[C@@H]2C[C@]2(c2ccc(-c3cc(-c4ccc5c(=O)[nH]cc(F)c5c4)c(N)nc3F)cc2)C1] | A | |
| I-471 | 6-(2-amino-6-fluoro-5-(4- ((1S,5R)-3-methyl-3- azabicyclo[3.1.0]hexan- 1-yl)phenyl)pyridin-3-yl)- 4-methylisoquinolin- 1(2H)-one | 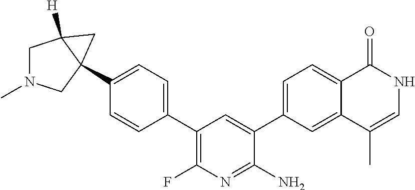 [SMILES: Cc1c[nH]c(=O)c2ccc(-c3cc(-c4ccc([C@]56C[C@H]5CN(C)C6)cc4)c(F)nc3N)cc12] | A | |
| I-472 | 7-(2-amino-5-(4-(4- cyclobutylpiperazin-1- yl)phenyl)-6- fluoropyridin-3-yl)-2- methylquinazolin-4(3H)- one | 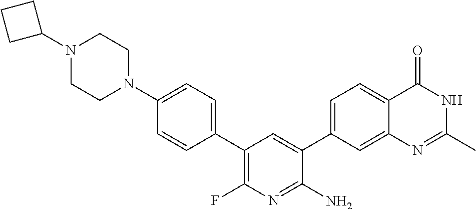 [SMILES: Cc1nc2cc(-c3cc(-c4ccc(N5CCN(C6CCC6)CC5)cc4)c(F)nc3N)ccc2c(=O)[nH]1] | A | |
| I-473 | 7-(2-amino-5-(4-(4- cyclobutylpiperazin-1- yl)phenyl)-6- fluoropyridin-3-yl)-5- fluoro-2- methylquinazolin-4(3H)- one | 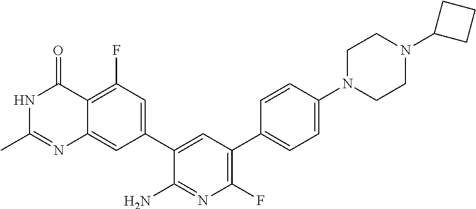 [SMILES: Cc1nc2cc(-c3cc(-c4ccc(N5CCN(C6CCC6)CC5)cc4)c(F)nc3N)cc(F)c2c(=O)[nH]1] | A | |
| I-474 | 7-(2-amino-5-(3- ((dimethylamino)methyl)- 4-morpholinophenyl)-6- fluoropyridin-3-yl)-2- methylquinazolin-4(3H)- one | 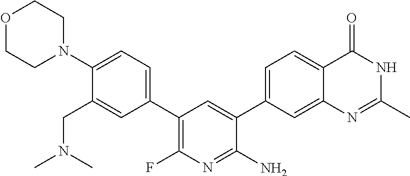 [SMILES: Cc1nc2cc(-c3cc(-c4ccc(N5CCOCC5)c(CN(C)C)c4)c(F)nc3N)ccc2c(=O)[nH]1] | B | |
| I-475 | 7-(2-amino-5-(3- ((dimethylamino)methyl)- 4-morpholinpophenyl)-6- fluoropyridin-3-yl)-5- fluoro-2- methylquinazolin-4(3H)- one | 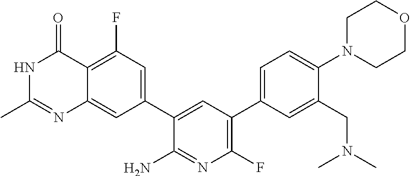 [SMILES: Cc1nc2cc(-c3cc(-c4ccc(N5CCOCC5)c(CN(C)C)c4)c(F)nc3N)cc(F)c2c(=O)[nH]1] | B | |
| I-476 | 6-(2-amino-5-(4-(4-(3,3- difluoropropyl)piperazin- 1-yl)phenyl)-6- fluoropyridin-3-yl)-3,4- dihydroisoquinolin- 1(2H)-one | 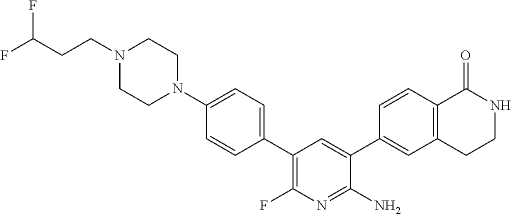 [SMILES: Nc1nc(F)c(-c2ccc(N3CCN(CCC(F)F)CC3)cc2)cc1-c1ccc2c(c1)CCNC2=O] | A | |
| I-477 | 6-(2-amino-5-(4-(4-(3,3- difluoropropyl)piperazin- 1-yl)phenyl)-6- fluoropyridin-3-yl)-7- fluoro-3,4- dihydroisoquinolin- 1(2H)-one | 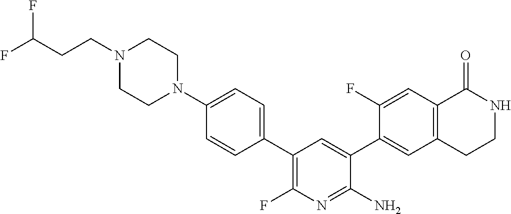 [SMILES: Nc1nc(F)c(-c2ccc(N3CCN(CCC(F)F)CC3)cc2)cc1-c1cc2c(cc1F)C(=O)NCC2] | A | |
| I-478 | 6-(2-amino-5-(4-(3,6- dihydro-2H-pyran-4-yl)- 2-(5-oxopyrrolidin-3- yl)phenyl)-6- fluoropyridin-3-yl)-7- fluoro-3,4- dihydroisoquinolin- 1(2H)-one | 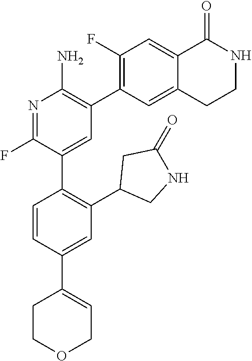 [SMILES: Nc1nc(F)c(-c2ccc(C3=CCOCC3)cc2C2CNC(=O)C2)cc1-c1cc2c(cc1F)C(=O)NCC2] | D | |
| I-479 | 6-(2-amino-6-fluoro-5-(2- (5-oxopyrrolidin-3-yl)-4- (tetrahydro-2H-pyran-4- yl)phenyl)pyridin-3-yl)-7- fluoro-3,4- dihydroisoquinolin- 1(2H)-one | 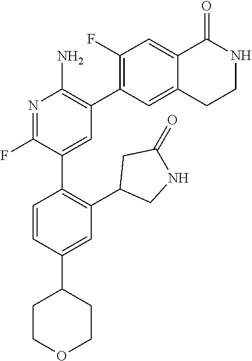 [SMILES: Nc1nc(F)c(-c2ccc(C3CCOCC3)cc2C2CNC(=O)C2)cc1-c1cc2c(cc1F)C(=O)NCC2] | D | |
| I-480 | 6-(2-amino-5-(3- ((dimethyalmino)methyl)- 4-morpholinophenyl)-6- fluoropyridin-3-yl)-4- fluoroisoquinolin-1(2H)- one | 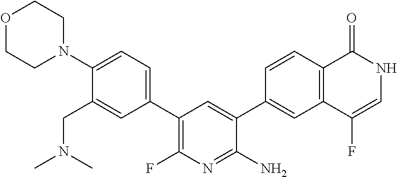 [SMILES: CN(C)Cc1cc(-c2cc(-c3ccc4c(=O)[nH]cc(F)c4c3)c(N)nc2F)ccc1N1CCOCC1] | B | |
| I-481 | 6-(2-amino-6-fluoro-5-(4- (4-isopropylpiperazin-1- yl)phenyl)pyridin-3-yl)- 4,8-difluoro-3- methylisoquinolin-1(2H)- one | 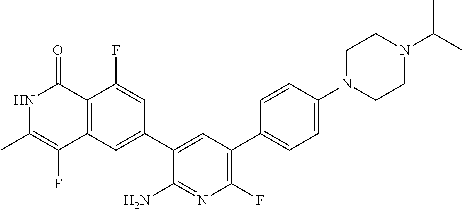 [SMILES: Cc1[nH]c(=O)c2c(F)cc(-c3cc(-c4ccc(N5CCN(C(C)C)CC5)cc4)c(F)nc3N)cc2c1F] | A | D |
| I-482 | 6-(2-amino-6-fluoro-5-(4- (4-isopropylpiperazin-1- yl)phenyl)pyridin-3-yl)- 4,8-difluoroisoquinolin- 1(2H)-one | 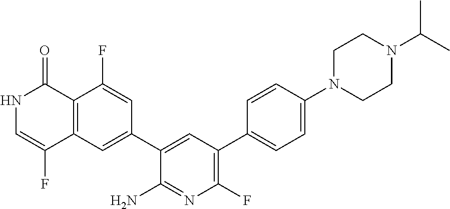 [SMILES: CC(C)N1CCN(c2ccc(-c3cc(-c4cc(F)c5c(=O)[nH]cc(F)c5c4)c(N)nc3F)cc2)CC1] | A | |
| I-483 | 6-(2-amino-5-(4-(4- (cyclopropylmethyl)piper- azin-1-yl)phenyl)-6- fluoropyridin-3-yl)-4,8- difluoroisoquinolin- 1(2H)-one | 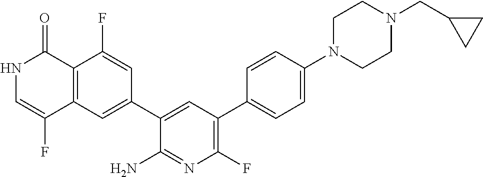 [SMILES: Nc1nc(F)c(-c2ccc(N3CCN(CC4CC4)CC3)cc2)cc1-c1cc(F)c2c(=O)[nH]cc(F)c2c1] | A | |
| I-484 | 7-(2-amino-5-(4-(4-(4,4- difluorobutyl)piperazin-1- yl)phenyl)-6- fluoropyridin-3-yl)-2- methylquinazolin-4(3H)- one | 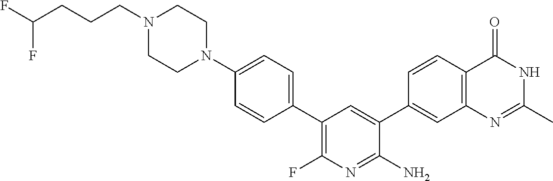 [SMILES: Cc1nc2cc(-c3cc(-c4ccc(N5CCN(CCCC(F)F)CC5)cc4)c(F)nc3N)ccc2c(=O)[nH]1] | A | |
| I-485 | 7-(2-amino-5-(4-(4-(3,3- difluoropropyl)piperazin- 1-yl)phenyl)-6- fluoropyridin-3-yl)-5- fluoro-2- methylquinazolin-4(3H)- one | 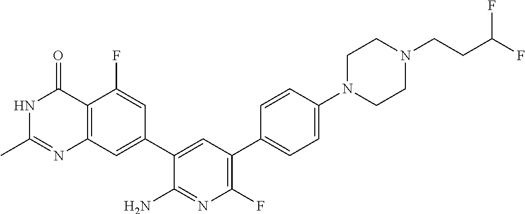 [SMILES: Cc1nc2cc(-c3cc(-c4ccc(N5CCN(CCC(F)F)CC5)cc4)c(F)nc3N)cc(F)c2c(=O)[nH]1] | B | |
| I-486 | 6-(2-amino-5-(3- ((dimethylamino)methyl)- 4-morpholinophenyl)-6- fluoropyridin-3-yl)-8- fluoro-3,4- dihydroisoquinolin- 1(2H)-one | 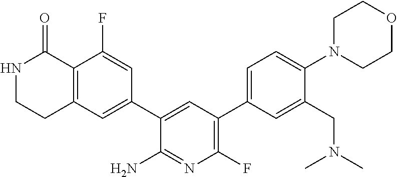 [SMILES: CN(C)Cc1cc(-c2cc(-c3cc(F)c4c(c3)CCNC4=O)c(N)nc2F)ccc1N1CCOCC1] | A | |
| I-487 | 6-(2-amino-5-(3- ((dimethylamino)methyl)- 4-thiomorpholinophenyl)- 6-fluoropyridin-3-yl)-3,4- dihydroisoquinolin- 1(2H)-one | 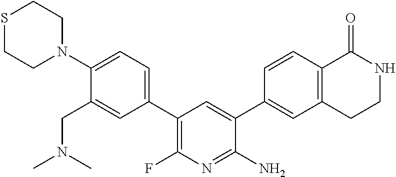 [SMILES: CN(C)Cc1cc(-c2cc(-c3ccc4c(c3)CCNC4=O)c(N)nc2F)ccc1N1CCSCC1] | A | |
| I-488 | 6-(2-amino-5-(3- ((dimethylamino)methyl)- 4-thiomorpholinophenyl)- 6-fluoropyridin-3-yl)-7- fluoro-3,4- dihydroisoquinolin- 1(2H)-one | 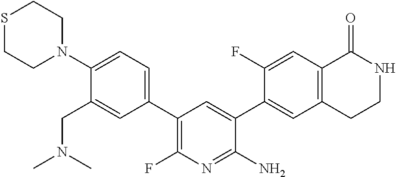 [SMILES: CN(C)Cc1cc(-c2cc(-c3cc4c(cc3F)C(=O)NCC4)c(N)nc2F)ccc1N1CCSCC1] | A | |
| I-489 | 6-(2-amino-6-fluoro-5-(4- (4-isopropylpiperazin-1- yl)phenyl)pyridin-3-yl)-4- chloro-8- fluoroisoquinolin-1(2H)- one | 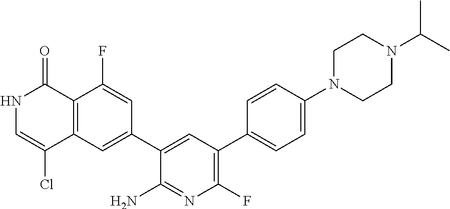 [SMILES: CC(C)N1CCN(c2ccc(-c3cc(-c4cc(F)c5c(=O)[nH]cc(Cl)c5c4)c(N)nc3F)cc2)CC1] | A | |
| I-490 | 7-(2-amino-5-(3- ((dimethylamino)methyl)- 4-(tetrahydro-2H-pyran- 4-yl)phenyl)-6- fluoropyridin-3-yl)-5- fluoro-2- methylquinazolin-4(3H)- one | 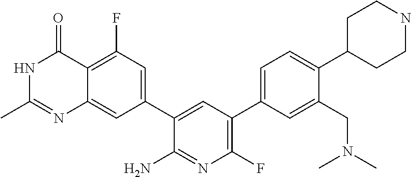 [SMILES: Cc1nc2cc(-c3cc(-c4ccc(C5CCNCC5)c(CN(C)C)c4)c(F)nc3N)cc(F)c2c(=O)[nH]1] | B | |
| I-491 | 6-(2-amino-6-fluoro-5-(4- morpholino-3-(pyrrolidin- 1- ylmethyl)phenyl)pyridin- 3-yl)-3,4- dihydroisoquinolin- 1(2H)-one | 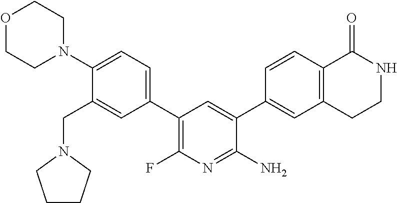 [SMILES: Nc1nc(F)c(-c2ccc(N3CCOCC3)c(CN3CCCC3)c2)cc1-c1ccc2c(c1)CCNC2=O] | B | |
| I-492 | 6-(2-amino-6-fluoro-5-(4- morpholino-3-(pyrrolidin- 1-ylmethyl)phenyl)pyridin- 3-yl)-7-fluoro-3,4- dihydroisoquinolin-1(2H)- one | 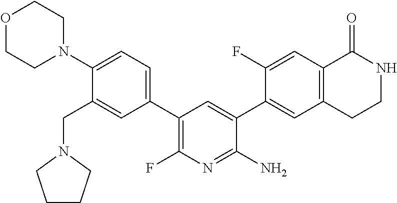 [SMILES: Nc1nc(F)c(-c2ccc(N3CCOCC3)c(CN3CCCC3)c2)cc1-c1cc2c(cc1F)C(=O)NCC2] | B | |
| I-493 | 6-(2-amino-5-(3- ((dimethylamino)methyl)- 4-(1,1- dioxidothiomorpholino)phe- nyl)-6-fluoropyridin-3- yl)-3,4- dihydroisoquinolin- 1(2H)-one | 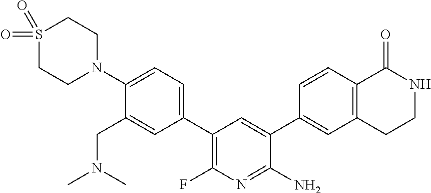 [SMILES: CN(C)Cc1cc(-c2cc(-c3ccc4c(c3)CCNC4=O)c(N)nc2F)ccc1N1CCS(=O)(=O)CC1] | B | |
| I-494 | 6-(2-amino-5-(4-(4- cyclopropylpiperazin-1- yl)-3- ((dimethylamino)methyl) phenyl)-6-fluoropyridin- 3-yl)-7-fluoro-3,4- dihydroisoquinolin- 1(2H)-one | 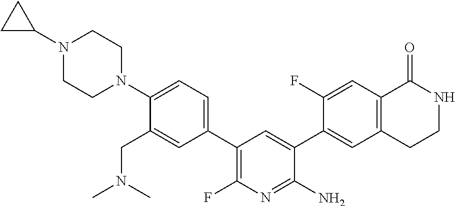 [SMILES: CN(C)Cc1cc(-c2cc(-c3cc4c(cc3F)C(=O)NCC4)c(N)nc2F)ccc1N1CCN(C2CC2)CC1] | B | |
| I-495 | 6-(2-amino-5-(4-(4- cyclopropylpiperazin-1- yl)-3- ((dimethylamino)methyl) phenyl)-6-fluoropyridin- 3-yl)-3,4- dihydroisoquinolin- 1(2H)-one | 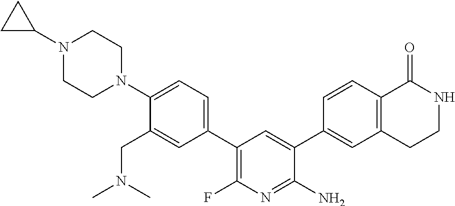 [SMILES: CN(C)Cc1cc(-c2cc(-c3ccc4c(c3)CCNC4=O)c(N)nc2F)ccc1N1CCN(C2CC2)CC1] | B | |
| I-496 | 6-(2-amino-6-fluoro-5-(4- morpholino-3- (morpholinomethyl)phenyl) pyridin-3-yl)-3,4- dihydroisoquinolin- 1(2H)-one | 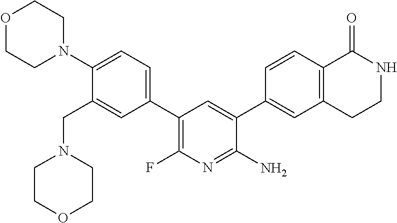 [SMILES: Nc1nc(F)c(-c2ccc(N3CCOCC3)c(CN3CCOCC3)c2)cc1-c1ccc2c(c1)CCNC2=O] | B | |
| I-497 | 6-(2-amino-6-fluoro-5-(4- morpholino-3- (morpholinomethyl)phenyl) pyridin-3-yl)-7-fluoro- 3,4-dihydroisoquinolin- 1(2H)-one | 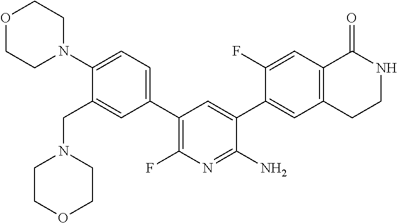 [SMILES: Nc1nc(F)c(-c2ccc(N3CCOCC3)c(CN3CCOCC3)c2)cc1-c1cc2c(cc1F)C(=O)NCC2] | B | |
| I-498 | 6-(2-amino-5-(3- ((dimethylamino)methyl)- 4-(1,1- dioxidothiomorpholino)phe- nyl)-6-fluoropyridin-3- yl)-7-fluoro-3,4- dihydroisoquinolin- 1(2H)-one | 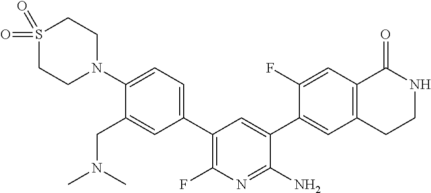 [SMILES: CN(C)Cc1cc(-c2cc(-c3cc4c(cc3F)C(=O)NCC4)c(N)nc2F)ccc1N1CCS(=O)(=O)CC1] | B | |
| I-499 | 6-(2-amino-6-fluoro-5-(4- ((3R,5S)-3,4,5- trimethylpiperazin-1- yl)phenyl)pyridin-3-yl)-4- fluoroisoquinolin-1(2H)- one | 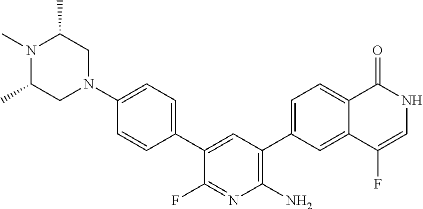 [SMILES: C[C@@H]1CN(c2ccc(-c3cc(-c4ccc5c(=O)[nH]cc(F)c5c4)c(N)nc3F)cc2)C[C@H](C)N1C] | A | |
| I-500 | 7-(2-amino-6-fluoro-5-(4- ((3R,5S)-3,4,5- trimethylpiperazin-1- yl)phenyl)pyridin-3-yl)-2- methylquinazolin-4(3H)- one | 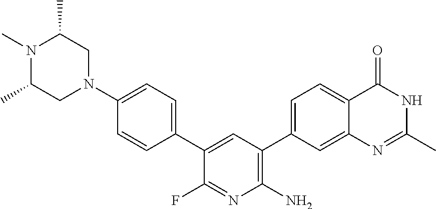 | B | |
| I-501 | 7-(2-amino-6-fluoro-5-(4- ((3R,5S)-3,4,5- trimethylpiperazin-1- yl)phenyl)pyridin-3-yl)-5- fluoro-2- methylquinazolin-4(3H)- one | 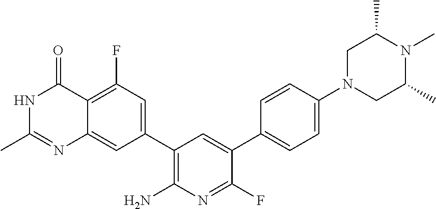 | B | |
| I-502 | 6-(2-amino-5-(3- ((dimethylamino)methyl)- 4-morpholinophenyl)-6- fluoropyridin-3-yl)-4- methylisoquinolin-1(2H)- one | 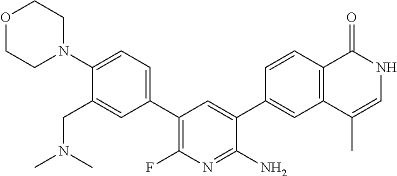 [SMILES: Cc1c[nH]c(=O)c2ccc(-c3cc(-c4ccc(N5CCOCC5)c(CN(C)C)c4)c(F)nc3N)cc12] | A | |
| I-503 | 6-(2-amino-5-(3- ((dimethylamino)methyl)- 4-(tetrahydro-2H-pyran- 4-yl)phenyl)-6- fluoropyridin-3-yl)-4- fluoroisoquinolin-1(2H)- one | 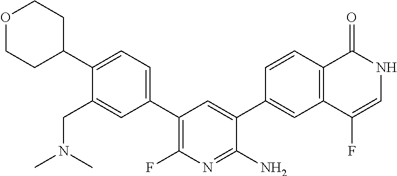 [SMILES: CN(C)Cc1cc(-c2cc(-c3ccc4c(=O)[nH]cc(F)c4c3)c(N)nc2F)ccc1C1CCOCC1] | A | D |
| I-504 | 6-(2-amino-5-(3- ((dimethylamino)methyl)- 4-(tetrahydro-2H-pyran- 4-yl)phenyl)-6- fluoropyriidn-3-yl)-8- fluoro-3- methylisoquinolin-1(2H)- one | 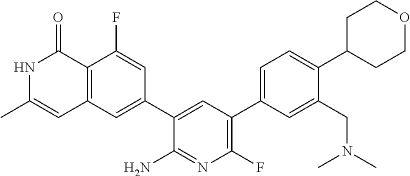 [SMILES: Cc1cc2cc(-c3cc(-c4ccc(C5CCOCC5)c(CN(C)C)c4)c(F)nc3N)cc(F)c2c(=O)[nH]1] | B | |
| I-505 | 6-(2-amino-5-(3- ((dimethylamino)methyl)- 4-morpholinophenyl)-6- fluoropyridin-3-yl)-8- fluoro-3- methylisoquinolin-1(2H)- one | 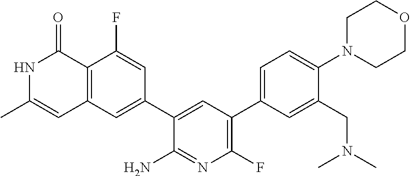 [SMILES: Cc1cc2cc(-c3cc(-c4ccc(N5CCOCC5)c(CN(C)C)c4)c(F)nc3N)cc(F)c2c(=O)[nH]1] | B | |
| I-506 | 6-(2-amino-5-(4- (cyclopropylmethoxy)-3- ((dimethylamino)methyl) phenyl)-6-fluoropyridin- 3-yl)-3,4- dihydroisoquinolin- 1(2H)-one | 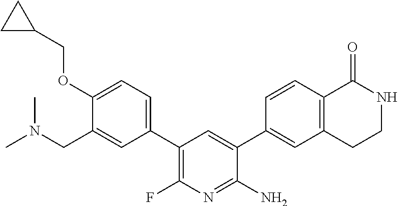 [SMILES: CN(C)Cc1cc(-c2cc(-c3ccc4c(c3)CCNC4=O)c(N)nc2F)ccc1OCC1CC1] | B | |
| I-507 | 6-(2-amino-5-(4- (cyclopropylmethoxy)-3- ((dimethylamino)methyl) phenyl)-6-fluoropyridin- 3-yl)-7-fluoro-3,4- dihydroisoquinolin- 1(2H)-one | 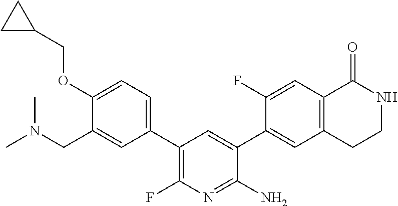 [SMILES: CN(C)Cc1cc(-c2cc(-c3cc4c(cc3F)C(=O)NCC4)c(N)nc2F)ccc1OCC1CC1] | B | |
| I-508 | 6-(2-amino-5-(4- (cyclopropylmethoxy)-3- ((4-methoxypiperidin-1- yl)methyl)phenyl)-6- fluoropyridin-3-yl)-3,4- dihydroisoquinolin- 1(2H)-one | 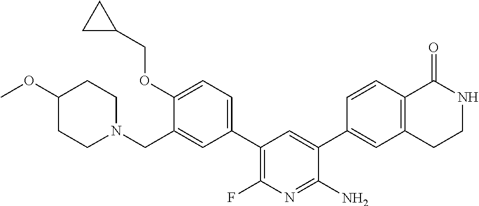 [SMILES: COC1CCN(Cc2cc(-c3cc(-c4ccc5c(c4)CCNC5=O)c(N)nc3F)ccc2OCC2CC2)CC1] | B | |
| I-509 | 6-(2-amino-5-(4- (cyclopropylmethoxy)-3- ((4-methoxypiperidin-1- yl)methyl)phenyl)-6- fluoropyridin-3-yl)-7- fluoro-3,4- dihydroisoquinolin- 1(2H)-one | 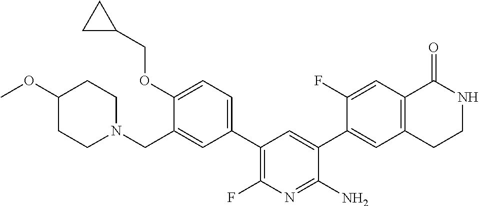 [SMILES: COC1CCN(Cc2cc(-c3cc(-c4cc5c(cc4F)C(=O)NCC5)c(N)nc3F)ccc2OCC2CC2)CC1] | B | |
| I-510 | 6-(2-amino-6-fluoro-5-(3- ((4-methoxypiperidin-1- yl)methyl)-4- morpholinophenyl)pyridin- 3-yl)-3,4- dihydroisoquinolin- 1(2H)-one | 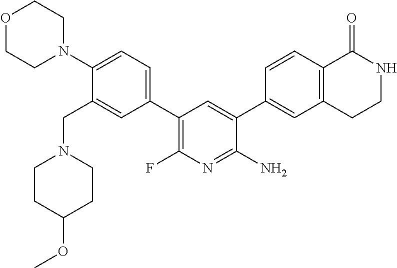 [SMILES: COC1CCN(Cc2cc(-c3cc(-c4ccc5c(c4)CCNC5=O)c(N)nc3F)ccc2N2CCOCC2)CC1] | B | |
| I-511 | 6-(2-amino-6-fluoro-5-(3- ((4-methoxypiperidin-1- yl)methyl)-4- morpholinophenyl)pyridin- 3-yl)-7-fluoro-3,4- dihydroisoquinolin- 1(2H)-one | 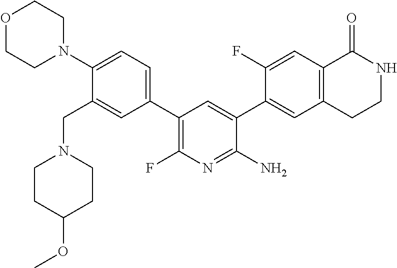 [SMILES: COC1CCN(Cc2cc(-c3cc(-c4cc5c(cc4F)C(=O)NCC5)c(N)nc3F)ccc2N2CCOCC2)CC1] | B | |
| I-512 | 6-(2-amino-6-fluoro-5-(3- ((4-methoxypiperidin-1- yl)methyl)-4- thiomorpholinophenyl)py- ridin-3-yl)-3,4- dihydroisoquinolin- 1(2H)-one | 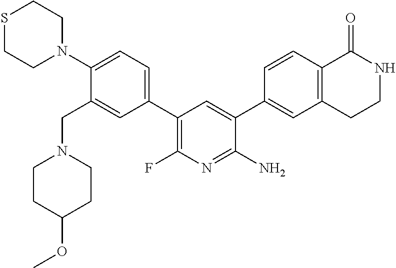 [SMILES: COC1CCN(Cc2cc(-c3cc(-c4ccc5c(c4)CCNC5=O)c(N)nc3F)ccc2N2CCSCC2)CC1] | B | |
| I-513 | 6-(2-amino-6-fluoro-5-(3- ((4-methoxypiperidin-1- yl)methyl)-4- thiomorpholinophenyl)py- ridin-3-yl)-7-fluoro-3,4- dihydroisoquinolin- 1(2H)-one | 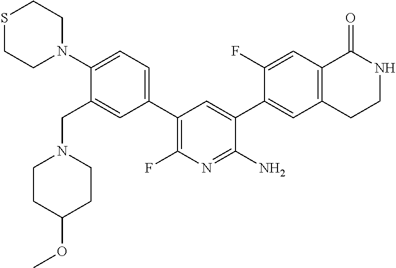 [SMILES: COC1CCN(Cc2cc(-c3cc(-c4cc5c(cc4F)C(=O)NCC5)c(N)nc3F)ccc2N2CCSCC2)CC1] | B | |
| I-514 | 2-(6-amino-2-fluoro-5-(7- fluoro-1-oxo-1,2,3,4- tetrahydroisoquinolin-6- yl)pyridin-3-yl)-5-(3,6- dihydro-2H-pyran-4- y)benzaldehyde | 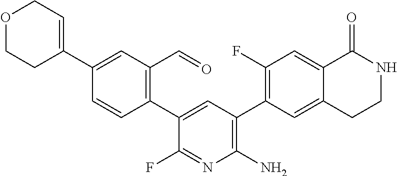 [SMILES: Nc1nc(F)c(-c2ccc(C3=CCOCC3)cc2C=O)cc1-c1cc2c(cc1F)C(=O)NCC2] | C | |
| I-515 | 6-(2-amino-5-(3- ((dimethylamino)methyl)- 4-morpholinophenyl)-6- fluoropyridin-3-yl)-4- fluoro-3- methylisoquinolin-1(2H)- one | 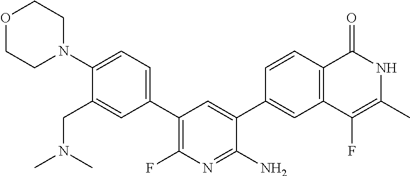 [SMILES: Cc1[nH]c(=O)c2ccc(-c3cc(-c4ccc(N5CCOCC5)c(CN(C)C)c4)c(F)nc3N)cc2c1F] | B | |
| I-516 | 6-(2-amino-6-fluoro-5-(4- methoxy-3-((3- methoxyazetidin-1- yl)methyl)phenyl)pyridin- 3-yl)-3,4- dihydroisoquinolin- 1(2H)-one | 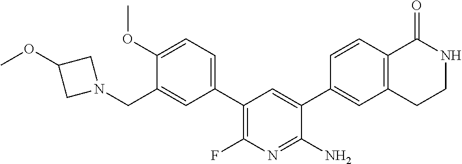 [SMILES: COc1ccc(-c2cc(-c3ccc4c(c3)CCNC4=O)c(N)nc2F)cc1CN1CC(OC)C1] | B | |
| I-517 | 6-(2-amino-6-fluoro-5-(4- methoxy-3-((3- methoxyazetidin-1- yl)methyl)phenyl)pyridin- 3-yl)-7-fluoro-3,4- dihydroisoquinolin- (2H)-one | 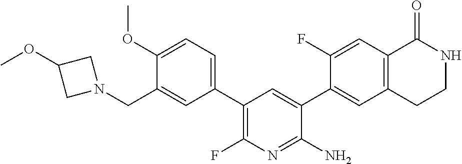 [SMILES: COc1ccc(-c2cc(-c3cc4c(cc3F)C(=O)NCC4)c(N)nc2F)cc1CN1CC(OC)C1] | B | |
| I-518 | 6-(2-amino-6-fluoro-5-(4- methoxy-3-((4- methoxypiperidin-1- yl)methyl)phenyl)pyridin- 3-yl)-3,4- dihydroisoquinolin- 1(2H)-one | 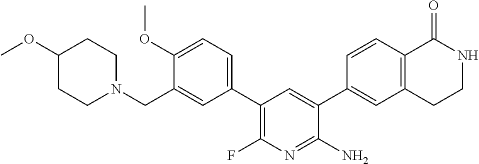 [SMILES: COc1ccc(-c2cc(-c3ccc4c(c3)CCNC4=O)c(N)nc2F)cc1CN1CCC(OC)CC1] | B | |
| I-519 | 6-(2-amino-6-fluoro-5-(4- methoxy-3-((4- methoxypiperidin-1- yl)methyl)phenyl)pyridin- 3-yl)-7-fluoro-3,4- dihydroisoquinolin- 1(2H)-one | 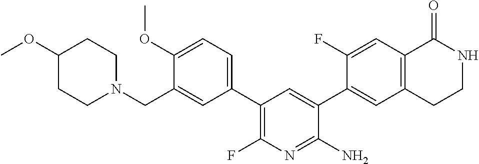 [SMILES: COc1ccc(-c2cc(-c3cc4c(cc3F)C(=O)NCC4)c(N)nc2F)cc1CN1CCC(OC)CC1] | B | |
| I-520 | 6-(2-amino-5-(4- ((1S,5R)-3-cyclobutyl-3- azabicyclo[3.1.0]hexan- 1-yl)phenyl)-6- fluoropyridin-3-yl)-4- fluoroisoquinolin-1(2H)- one | 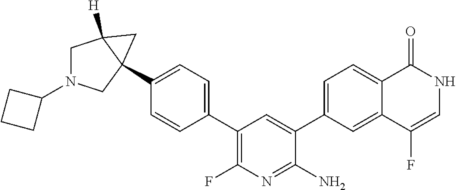 [SMILES: Nc1nc(F)c(-c2ccc([C@]34C[C@H]3CN(C3CCC3)C4)cc2)cc1-c1ccc2c(=O)[nH]cc(F)c2c1] | A | |
| I-521 | 6-(2-amino-5-(3- ((dimethylamino)methyl)- 4-(tetrahydro-2H-pyran- 4-yl)phenyl)-6- fluoropyridin-3-yl)-4- fluoro-3- methylisoquinolin-1(2H)- one | 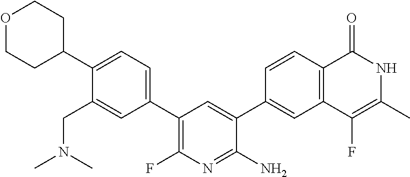 [SMILES: Cc1[nH]c(=O)c2ccc(-c3cc(-c4ccc(C5CCOCC5)c(CN(C)C)c4)c(F)nc3N)cc2c1F] | B | |
| I-522 | 6-(2-amino-6-fluoro-5-(3- ((3-methoxyazetidin-1- yl)methyl)-4- morpholinophenyl)pyridin- 3-yl)-3,4- dihydroisoquinolin- 1(2H)-one | 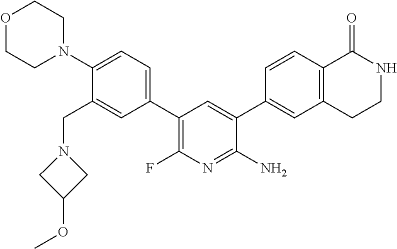 [SMILES: COC1CN(Cc2cc(-c3cc(-c4ccc5c(c4)CCNC5=O)c(N)nc3F)ccc2N2CCOCC2)C1] | B | |
| I-523 | 6-(2-amino-6-fluoro-5-(3- ((3-methoxyazetidin-1- yl)methyl)-4- morpholinophenyl)pyridin- 3-yl)-7-fluoro-3,4- dihydroisoquinolin- 1(2H)-one | 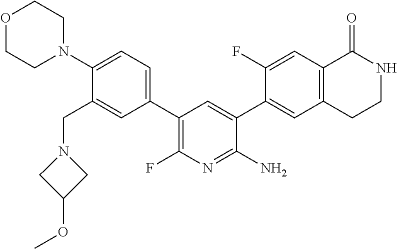 [SMILES: COC1CN(Cc2cc(-c3cc(-c4cc5c(cc4F)C(=O)NCC5)c(N)nc3F)ccc2N2CCOCC2)C1] | B | |
| I-524 | 6-(2-amino-5-(4-(4,4- difluorobutoxy)-3- ((dimethylamino)methyl) phenyl)-6-fluoropyridin- 3-yl)-3,4- dihydroisoquinolin- 1(2H)-one | 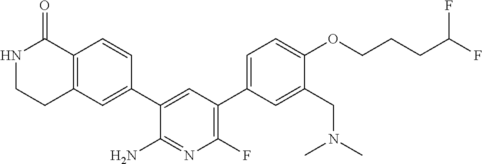 [SMILES: CN(C)Cc1cc(-c2cc(-c3ccc4c(c3)CCNC4=O)c(N)nc2F)ccc1OCCCC(F)F] | A | |
| I-525 | 6-(2-amino-5-(4-(4,4- difluorobutoxy)-3- ((dimethylamino)methyl) phenyl)-6-fluoropyridin- 3-yl)-7-fluoro-3,4- dihydroisoquinolin- 1(2H)-one | 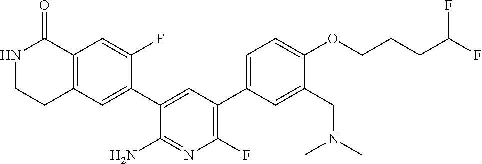 [SMILES: CN(C)Cc1cc(-c2cc(-c3cc4c(cc3F)C(=O)NCC4)c(N)nc2F)ccc1OCCCC(F)F] | B | |
| I-526 | 6-(2-amino-5-(4-(4,4- difluorobutoxy)-3-((4- methoxypiperidin-1- yl)methyl)phenyl)-6- fluoropyridin-3-yl)-3,4- dihydroisoquinolin- 1(2H)-one | 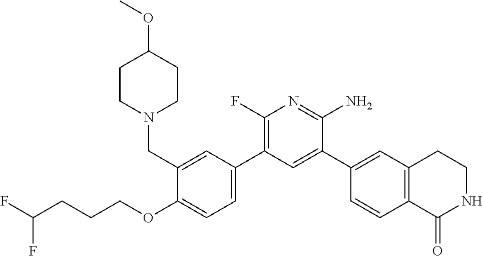 [SMILES: COC1CCN(Cc2cc(-c3cc(-c4ccc5c(c4)CCNC5=O)c(N)nc3F)ccc2OCCCC(F)F)CC1] | B | |
| I-527 | 6-(2-amino-5-(4-(4,4- difluorobutoxy)-3-((4- methoxypiperidin-1- yl)methyl)phenyl)-6- fluoropyridin-3-yl)-7- fluoro-3,4- dihydroisoquinolin- 1(2H)-one | 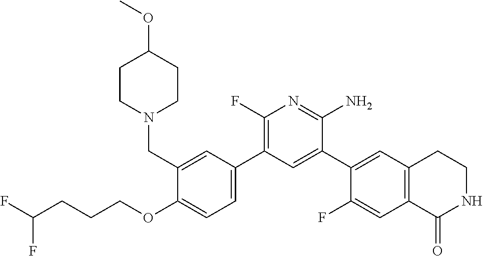 [SMILES: COC1CCN(Cc2cc(-c3cc(-c4cc5c(cc4F)C(=O)NCC5)c(N)nc3F)ccc2OCCCC(F)F)CC1] | B | |
| I-528 | 6-(2-amino-5-(4-(3,6- dihydro-2H-pyran-4-yl)- 2- (hydroxymethyl)phenyl)- 6-fluoropyridin-3-yl)-7- fluoro-3,4- dihydroisoquinolin- 1(2H)-one | 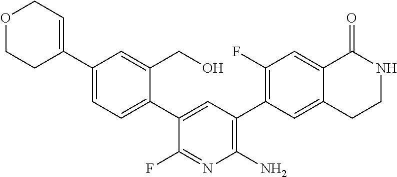 [SMILES: Nc1nc(F)c(-c2ccc(C3=CCOCC3)cc2CO)cc1-c1cc2c(cc1F)C(=O)NCC2] | C | |
| I-529 | 6-(2-amino-5-(4-(4- (cyclopropylmethyl)piper- azin-1-yl)phenyl)-6- fluoropyridin-3-yl)-4,8- difluoro-3- methylisoquinolin-1(2H)- one | 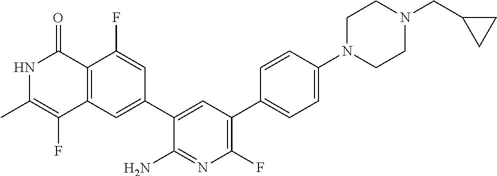 [SMILES: Cc1[nH]c(=O)c2c(F)cc(-c3cc(-c4ccc(N5CCN(CC6CC6)CC5)cc4)c(F)nc3N)cc2c1F] | A | |
| I-530 | 6-(2-amino-5-(3- ((dimethylamino)methyl)- 4-((tetrahydro-2H-pyran- 4-yl)oxy)phenyl)-6- fluoropyriidn-3-yl)-3,4- dihydroisoquinolin- 1(2H)-one | 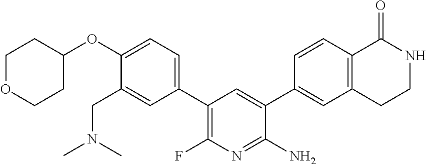 [SMILES: CN(C)Cc1cc(-c2cc(-c3ccc4c(c3)CCNC4=O)c(N)nc2F)ccc1OC1CCOCC1] | B | |
| I-531 | 6-(2-amino-5-(3- ((dimethylamino)methyl)- 4-((tetrahydro-2H-pyran- 4-yl)oxy)phenyl)-6- fluoropyridin-3-yl)-7- fluoro-3,4- dihydroisoquinolin- 1(2H)-one | 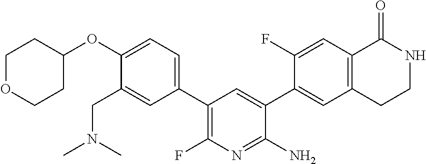 [SMILES: CN(C)Cc1cc(-c2cc(-c3cc4c(cc3F)C(=O)NCC4)c(N)nc2F)ccc1OC1CCOCC1] | B | |
| I-532 | 6-(2-amino-5-(3- ((dimethylamino)methyl)- 4-((tetrahydro-2H-pyran- 4-yl)methoxy)phenyl)-6- fluoropyridin-3-yl)-3,4- dihydroisoquinolin- 1(2H)-one | 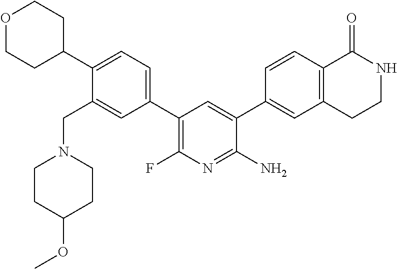 [SMILES: COC1CCN(Cc2cc(-c3cc(-c4ccc5c(c4)CCNC5=O)c(N)nc3F)ccc2C2CCOCC2)CC1] | B | |
| I-533 | 6-(2-amino-5-(3- ((dimethylamino)methyl)- 4-((tetrahydro-2H-pyran- 4-yl)methoxy)phenyl)-6- fluoropyridin-3-yl)-7- fluoro-3,4- dihydroisoquinolin- 1(2H)-one | 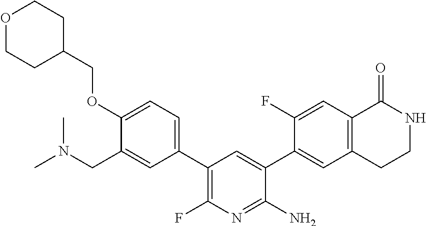 [SMILES: CN(C)Cc1cc(-c2cc(-c3cc4c(cc3F)C(=O)NCC4)c(N)nc2F)ccc1OCC1CCOCC1] | A | |
| I-534 | 6-(2-amino-5-(3- ((dimethylamino)methyl)- 4-morpholinophenyl)-6- fluoropyridin-3-yl)-5,8- difluoro-3- mehylisoquinolin-1(2H)- one | 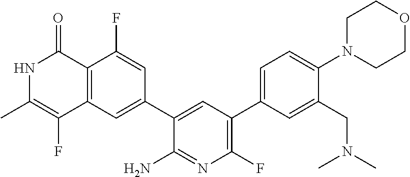 [SMILES: Cc1[nH]c(=O)c2c(F)cc(-c3cc(-c4ccc(N5CCOCC5)c(CN(C)C)c4)c(F)nc3N)cc2c1F] | B | |
| I-535 | 6-(2-amino-5-(3- ((dimethylamino)methyl)- 4-(tetrahydro-2H-pyran- 4-yl)phenyl)-6- fluoropyridin-3-yl)-4,8- difluoro-3- methylisoquinolin-1(2H)- one | 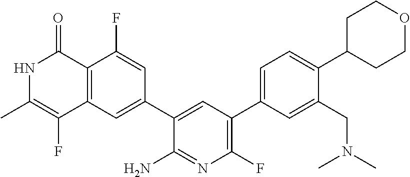 [SMILES: Cc1[nH]c(=O)c2c(F)cc(-c3cc(-c4ccc(C5CCOCC5)c(CN(C)C)c4)c(F)nc3N)cc2c1F] | B | |
| I-536 | 6-(2-amino-6-fluoro-5-(3- (hydroxymethyl)-4- methoxyphenyl)pyridin- 3-yl)-3,4- dihydroisoquinolin- 1(2H)-one | 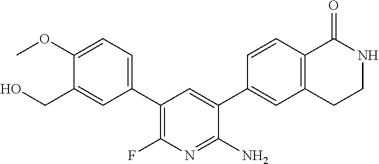 [SMILES: COc1ccc(-c2cc(-c3ccc4c(c3)CCNC4=O)c(N)nc2F)cc1CO] | B | |
| I-537 | 6-(2-amino-6-fluoro-5-(4- (4-isoproopylpiperazin-1- yl)phenyl)pyridin-3-yl)-4- methoxyisoquinolin- 1(2H)-one | 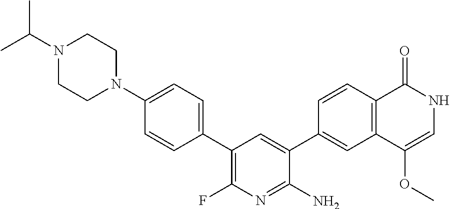 [SMILES: COc1c[nH]c(=O)c2ccc(-c3cc(-c4ccc(N5CCN(C(C)C)CC5)cc4)c(F)nc3N)cc12] | A | |
| I-538 | 6-(2-amino-6-fluoro-5-(4- (4-isopropylpiperazin-1- yl)phenyl)pyridin-3-yl)-4- chloro-3- methylisoquinolin-1(2H)- one | 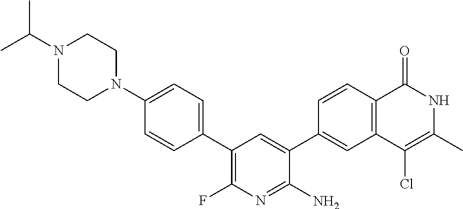 [SMILES: Cc1[nH]c(=O)c2ccc(-c3cc(-c4ccc(N5CCN(C(C)C)CC5)cc4)c(F)nc3N)cc2c1Cl] | A | |
| I-539 | 6-(2-amino-6-fluoro-5-(4- (8-methyl-5-oxa-2,8- diazaspiro[3.5]nonan-2- yl)phenyl)pyridin-3-yl)-7- fluoro-3,4- dihydroisoquinolin- 1(2H)-one | 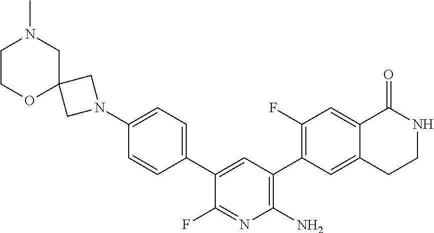 [SMILES: CN1CCOC2(C1)CN(c1ccc(-c3cc(-c4cc5c(cc4F)C(=O)NCC5)c(N)nc3F)cc1)C2] | A | |
| I-540 | 6-(2-amino-6-fluoro-5-(3- ((4-methoxypiperidin-1- yl)methyl)-4-(tetrahydro- 2H-pyran-4- yl)phenyl)pyridin-3-yl)- 3,4-dihydroisoquinolin- 1(2H)-one | 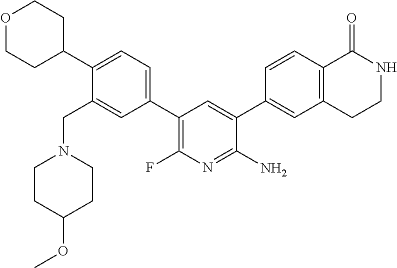 [SMILES: COC1CCN(Cc2cc(-c3cc(-c4ccc5c(c4)CCNC5=O)c(N)nc3F)ccc2C2CCOCC2)CC1] | B | |
| I-541 | 6-(2-amino-6-fluoro-5-(3- ((4-methoxypiperidin-1- yl)methyl)-4-(tetrahydro- 2H-pyran-4- yl)phenyl)pyridin-3-yl)-7- fluoro-3,4- dihydroisoquinolin- 1(2H)-one | 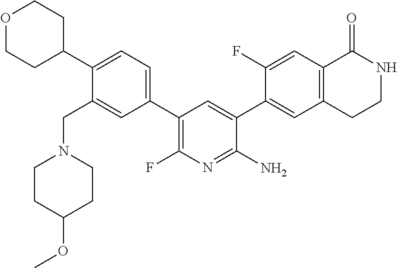 [SMILES: COC1CCN(Cc2cc(-c3cc(-c4cc5c(cc4F)C(=O)NCC5)c(N)nc3F)ccc2C2CCOCC2)CC1] | B | |
| I-542 | 6-(2-amino-6-fluoro-5-(3- ((3-methoxypyrrolidin-1- yl)methyl)-4- morpholinophenyl)pyridin- 3-yl)-3,4- dihydroisoquinolin- 1(2H)-one | 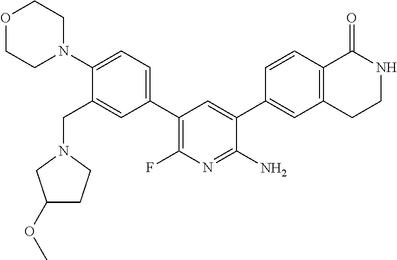 [SMILES: COC1CCN(Cc2cc(-c3cc(-c4ccc5c(c4)CCNC5=O)c(N)nc3F)ccc2N2CCOCC2)C1] | B | |
| I-543 | 6-(2-amino-6-fluoro-5-(3- ((3-methoxypyrrolidin-1- yl)methyl)-4- morpholinophenyl)pyridin- 3-yl)-7-fluoro-3,4- dihydroisoquinolin- 1(2H)-one | 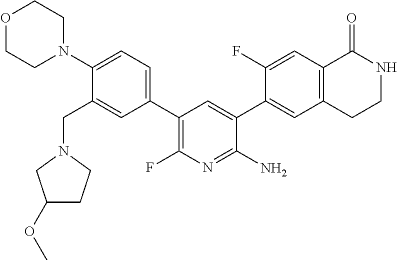 [SMILES: COC1CCN(Cc2cc(-c3cc(-c4cc5c(cc4F)C(=O)NCC5)c(N)nc3F)ccc2N2CCOCC2)C1] | A | B |
| I-544 | 6-(2-amino-5-(4-(4-(4,4- difluorobutyl)piperazin-1- yl)phenyl)-6- fluoropyridin-3-yl)-4- fluoroisoquinolin-1(2H)- one | 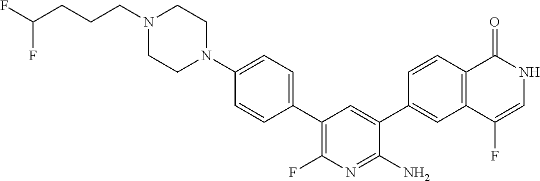 [SMILES: Nc1nc(F)c(-c2ccc(N3CCN(CCCC(F)F)CC3)cc2)cc1-c1ccc2c(=O)[nH]cc(F)c2c1] | A | |
| I-545 | 6-(2-amino-5-(4-(4- cyclobutylpiperazin-1- yl)phenyl)-6- fluoropyridin-3-yl)-4- methylisoquinolin-1(2H)- one | 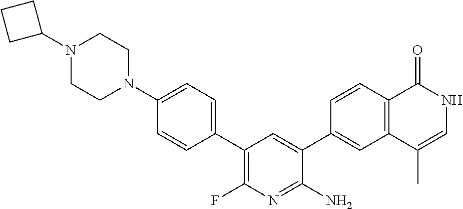 [SMILES: Cc1c[nH]c(=O)c2ccc(-c3cc(-c4ccc(N5CCN(C6CCC6)CC5)cc4)c(F)nc3N)cc12] | A | |
| I-546 | 6-(2-amino-5-(4-(4- cyclobutylpiperazin-1- yl)phenyl)-6- fluoropyridin-3-yl)-4- fluoroisoquinolin-1(2H)- one | 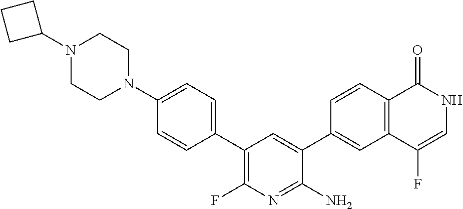 [SMILES: Nc1nc(F)c(-c2ccc(N3CCN(C4CCC4)CC3)cc2)cc1-c1ccc2c(=O)[nH]cc(F)c2c1] | A | |
| I-547 | 6-(2-amino-6-fluoro-5-(4- (4-isopropylpiperazin-1- yl)phenyl)pyridin-3-yl)- 4,7-difluoroisoquinolin- 1(2H)-one | 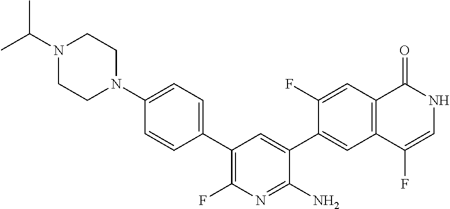 [SMILES: CC(C)N1CCN(c2ccc(-c3cc(-c4cc5c(F)c[nH]c(=O)c5cc4F)c(N)nc3F)cc2)CC1] | A | |
| I-548 | 6-(2-amino-5-(4-(4- (cyclopropylmethyl)piper- azin-1-yl)phenyl)-6- fluoropyridin-3-yl)-4,7- difluoroisoquinolin- 1(2H)-one | 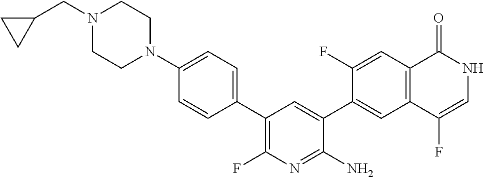 [SMILES: Nc1nc(F)c(-c2ccc(N3CCN(CC4CC4)CC3)cc2)cc1-c1cc2c(F)c[nH]c(=O)c2cc1F] | A | D |
| I-549 | 6-(2-amino-5-(3- (azetidin-1-ylmethyl)-4- morpholinophenyl)-6- fluoropyridin-3-yl)-3,4- dihydroisoquinolin- 1(2H)-one | 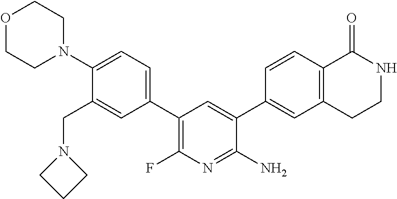 [SMILES: Nc1nc(F)c(-c2ccc(N3CCOCC3)c(CN3CCC3)c2)cc1-c1ccc2c(c1)CCNC2=O] | B | |
| I-550 | 6-(2-amino-5-(3- (azetidin-1-ylmethyl)-4- morpholinophenyl)-6- fluoropyridin-3-yl)-7- fluoro-3,4- dihydroisoquinolin- 1(2H)-one | 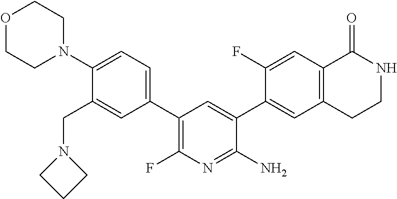 [SMILES: Nc1nc(F)c(-c2ccc(N3CCOCC3)c(CN3CCC3)c2)cc1-c1cc2c(cc1F)C(=O)NCC2] | B | |
| I-551 | 6-(6′-amino-6- ((dimethylamino)methyl)- 2′-fluoro-5-morpholino- [2,3′-bipyridin]-5′-yl)-3,4- dihydroisoquinolin- 1(2H)-one | 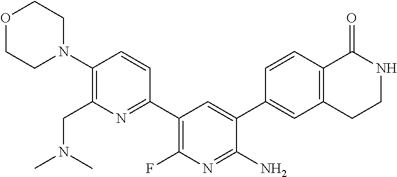 [SMILES: CN(C)Cc1nc(-c2cc(-c3ccc4c(c3)CCNC4=O)c(N)nc2F)ccc1N1CCOCC1] | A | D |
| I-552 | 6-(2-amino-5-(3- ((ethyl(methyl)amino)meth- yl)-4-(tetrahydro-2H- pyran-4-yl)phenyl)-6- fluoropyridin-3-yl)-7- fluoro-3,4- dihydroisoquinolin- 1(2H)-one | 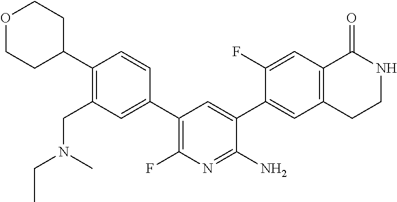 [SMILES: CCN(C)Cc1cc(-c2cc(-c3cc4c(cc3F)C(=O)NCC4)c(N)nc2F)ccc1C1CCOCC1] | B | |
| I-553 | 6-(2-amino-6-fluoro-5-(3- (((2- methoxyethyl)(methyl)a- mino)methyl)-4- morpholinophenyl)pyridin- 3-yl)-3,4- dihydroisoquinolin- 1(2H)-one | 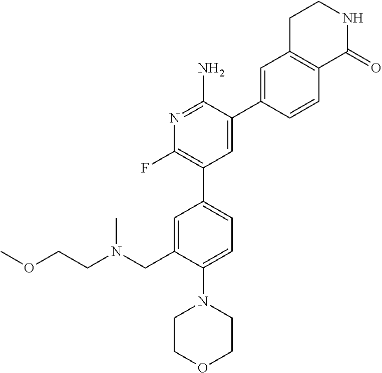 [SMILES: COCCN(C)Cc1cc(-c2cc(-c3ccc4c(c3)CCNC4=O)c(N)nc2F)ccc1N1CCOCC1] | A | |
| I-554 | 6-(2-amino-6-fluoro-5-(3- (((2- methoxyethyl)(methyl)a- mino)methyl)-4- morpholinophenyl)pyridin- 3-yl)-7-fluoro-3,4- dihydroisoquinolin- 1(2H)-one | 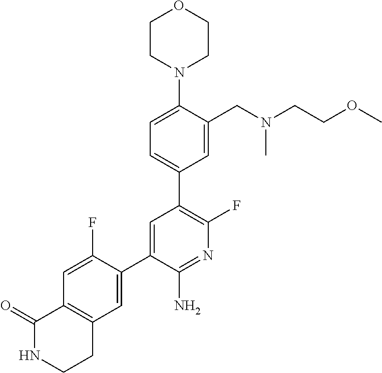 [SMILES: COCCN(C)Cc1cc(-c2cc(-c3cc4c(cc3F)C(=O)NCC4)c(N)nc2F)ccc1N1CCOCC1] | B | |
| I-555 | 6-(2-amino-5-(4-(2,2- difluoroethoxy)-3- ((dimethylamino)methyl) phenyl)-6-fluoropyridin- 3-yl)-3,4- dihydroisoquinolin- 1(2H)-one | 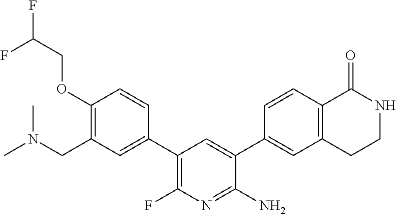 [SMILES: CN(C)Cc1cc(-c2cc(-c3ccc4c(c3)CCNC4=O)c(N)nc2F)ccc1OCC(F)F] | B | |
| I-556 | 6-(2-amino-5-(4-(2,2- difluoroethoxy)-3- ((dimethylamino)methyl) phenyl)-6-fluoropyridin- 3-yl)-7-fluoro-3,4- dihydroisoquinolin- 1(2H)-one | 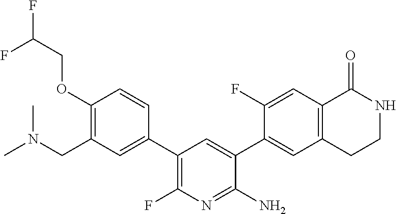 [SMILES: CN(C)Cc1cc(-c2cc(-c3cc4c(cc3F)C(=O)NCC4)c(N)nc2F)ccc1OCC(F)F] | B | |
| I-557 | 6-(2-amino-6-fluoro-5-(3- (pyrrolidin-1-ylmethyl)-4- (tetrahydro-2H-pyran-4- y)phenyl)pyridin-3-yl)- 3,4-dihydroisoquinolin- 1(2H)-one | 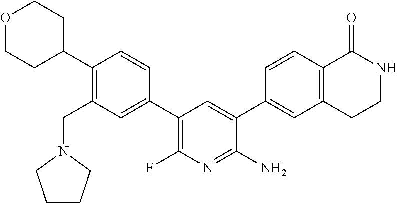 [SMILES: Nc1nc(F)c(-c2ccc(C3CCOCC3)c(CN3CCCC3)c2)cc1-c1ccc2c(c1)CCNC2=O] | B | |
| I-558 | 6-(2-amino-6-fluoro-5-(3- (pyrrolidin-1-ylmethyl)-4- (tetrahydro-2H-pyran-4- yl)phenyl)pyridin-3-yl)-7- fluoro-3,4- dihydroisoquinolin- 1(2H)-one | 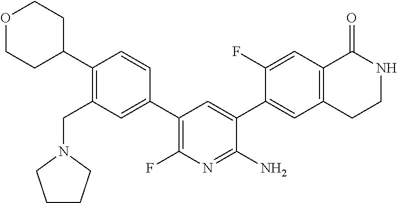 [SMILES: Nc1nc(F)c(-c2ccc(C3CCOCC3)c(CN3CCCC3)c2)cc1-c1cc2c(cc1F)C(=O)NCC2] | B | |
| I-559 | 6-(2-amino-6-fluoro-5-(4- morpholino-3-(piperidin- 1- ylmethyl)phenyl)pyridin- 3-yl)-3,4- dihydroisoquinolin- 1(2H)-one | 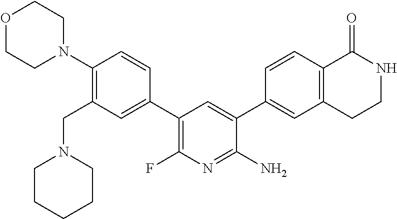 [SMILES: Nc1nc(F)c(-c2ccc(N3CCOCC3)c(CN3CCCCC3)c2)cc1-c1ccc2c(c1)CCNC2=O] | B | |
| I-560 | 6-(2-amino-6-fluoro-5-(4- morpholino-3-(piperidin- 1- ylmethyl)phenyl)pyridin- 3-yl)-7-fluoro-3,4- dihydroisoquinolin- 1(2H)-one | 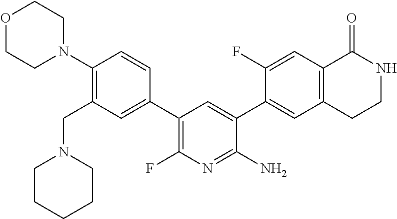 [SMILES: Nc1nc(F)c(-c2ccc(N3CCOCC3)c(CN3CCCCC3)c2)cc1-c1cc2c(cc1F)C(=O)NCC2] | B | |
| I-561 | 6-(2-amino-5-(3- ((dimethylamino)methyl)- 4-morpholinophenyl)-6- fluoropyridin-3-yl)-4,7- difluoroisoquinoiln- 1(2H)-one | 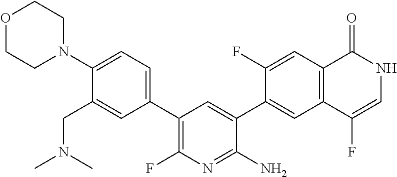 [SMILES: CN(C)Cc1cc(-c2cc(-c3cc4c(F)c[nH]c(=O)c4cc3F)c(N)nc2F)ccc1N1CCOCC1] | A | |
| I-562 | 6-(2-amino-5-(3- ((dimethylamino)methyl)- 4-(tetrahydro-2H-pyran- 4-yl)phenyl)-6- fluoropyridin-3-yl)-4,7- difluoroisoquinolin- 1(2H)-one | 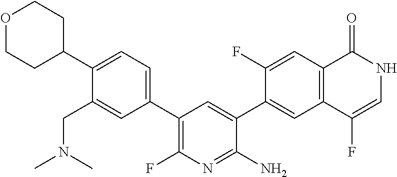 [SMILES: CN(C)Cc1cc(-c2cc(-c3cc4c(F)c[nH]c(=O)c4cc3F)c(N)nc2F)ccc1C1CCOCC1] | A | |
| I-563 | (R)-6-(2-amino-6-fluoro- 5-(3-((3- methoxypyrrolidin-1- yl)methyl)-4- morpholinophenyl)pyridin- 3-yl)-3,4- dihydroisoquinolin- 1(2H)-one | 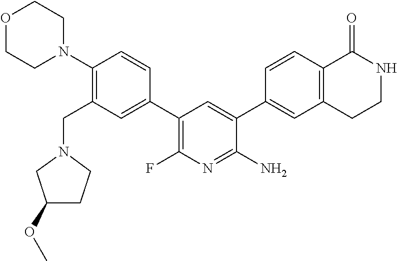 [SMILES: CO[C@@H]1CCN(Cc2cc(-c3cc(-c4ccc5c(c4)CCNC5=O)c(N)nc3F)ccc2N2CCOCC2)C1] | B | |
| I-564 | (R)-6-(2-amino-6-fluoro- 5-(3-((3- methoxypyrrolidin-1- yl)methyl)-4- morpholinophenyl)pyridin- 3-yl)-7-fluoro-3,4- dihydroisoquinolin- 1(2H)-one | 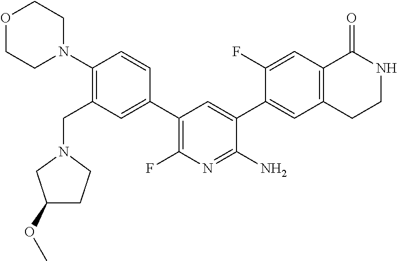 [SMILES: CO[C@@H]1CCN(Cc2cc(-c3cc(-c4cc5c(cc4F)C(=O)NCC5)c(N)nc3F)ccc2N2CCOCC2)C1] | B | |
| I-565 | (S)-6-(2-amino-6-fluoro- 5-(3-((3- methoxypyrrolidin-1- yl)methyl)-4- morpholinophenyl)pyridin- 3-yl)-3,4- dihydroisoquinolin- 1(2H)-one | 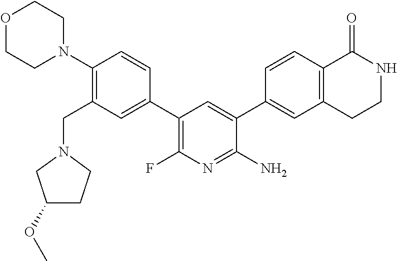 [SMILES: CO[C@H]1CCN(Cc2cc(-c3cc(-c4ccc5c(c4)CCNC5=O)c(N)nc3F)ccc2N2CCOCC2)C1] | A | |
| I-566 | (S)-6-(2-amino-6-fluoro- 5-(3-((3- methoxypyrrolidin-1- yl)methyl)-4- morpholinophenyl)pyridin- 3-yl)-7-fluoro-3,4- dihydroisoquinolin- 1(2H)-one | 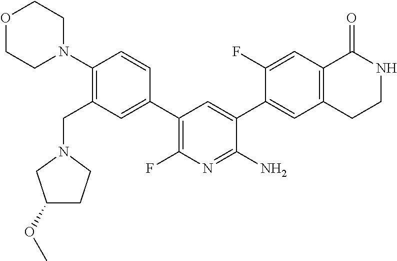 [SMILES: CO[C@H]1CCN(Cc2cc(-c3cc(-c4cc5c(cc4F)C(=O)NCC5)c(N)nc3F)ccc2N2CCOCC2)C1] | A | |
| I-567 | 6-(2-amino-5-(4-(4-(4,4- difluorobutyl)piperazin-1- yl)phenyl)-6- fluoropyridin-3-yl)-8- fluoro-3- methylisoquinolin-1(2H)- one | 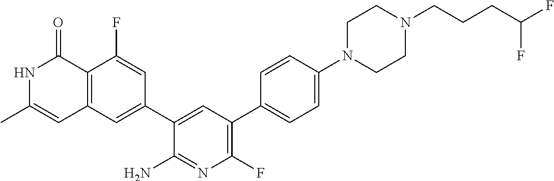 [SMILES: Cc1cc2cc(-c3cc(-c4ccc(N5CCN(CCCC(F)F)CC5)cc4)c(F)nc3N)cc(F)c2c(=O)[nH]1] | A | |
| I-568 | 6-(5-(4-((1- acetylpiperidin-4-yl)oxy)- 3- ((dimethylamino)methyl) phenyl)-2-amino-6- fluoropyridin-3-yl)-3,4- dihydroisoquinolin- 1(2H)-one | 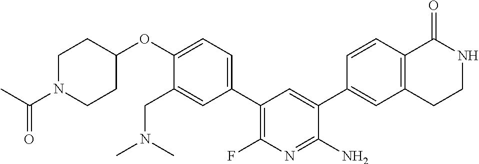 [SMILES: CC(=O)N1CCC(Oc2ccc(-c3cc(-c4ccc5c(c4)CCNC5=O)c(N)nc3F)cc2CN(C)C)CC1] | A | |
| I-569 | 6-(5-(4-((1- acetylpiperidin-4-yl)oxy)- 3- ((dimethylamino)methyl) phenyl)-2-amino-6- fluoropyridin-3-yl)-7- fluoro-3,4- dihydroisoquinolin- 1(2H)-one | 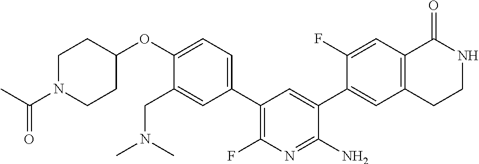 [SMILES: CC(=O)N1CCC(Oc2ccc(-c3cc(-c4cc5c(cc4F)C(=O)NCC5)c(N)nc3F)cc2CN(C)C)CC1] | B | |
| I-570 | 6-(2-amino-6-fluoro-5-(4- (4-isopropylpiperazin-1- yl)phenyl)pyridin-3-yl)-4- chloro-2,7-naphthyridin- 1(2H)-one | 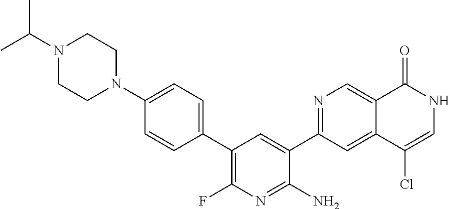 [SMILES: CC(C)N1CCN(c2ccc(-c3cc(-c4cc5c(Cl)c[nH]c(=O)c5cn4)c(N)nc3F)cc2)CC1] | A | |
| I-571 | 6-(2-amino-6-fluoro-5-(4- (4-isopropylpiperazin-1- yl)phenyl)pyridin-3-yl)-8- fluoro-4- methylisoquinolin-1(2H)- one | 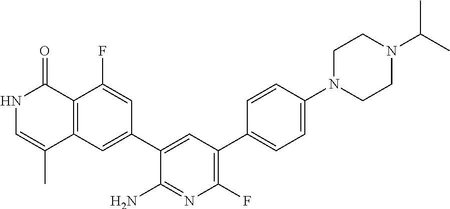 [SMILES: Cc1c[nH]c(=O)c2c(F)cc(-c3cc(-c4ccc(N5CCN(C(C)C)CC5)cc4)c(F)nc3N)cc12] | A | |
| I-572 | 6-(2-amino-6-fluoro-5-(4- (4-isopropylpiperazin-1- yl)phenyl)pyridin-3-yl)- 4,7-difluoro-3- methylisoquinolin-1(2H)- one | 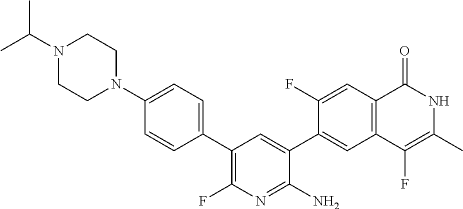 [SMILES: Cc1[nH]c(=O)c2cc(F)c(-c3cc(-c4ccc(N5CCN(C(C)C)CC5)cc4)c(F)nc3N)cc2c1F] | A | |
| I-573 | 6-(2-amino-6-fluoro-5-(3- (((2- methoxyethyl)(methyl)a- mino)methyl)-4- (tetrahydro-2H-pyran-4- yl)phenyl)pyridin-3-yl)- 3,4-dihydroisoquinolin- 1(2H)-one | 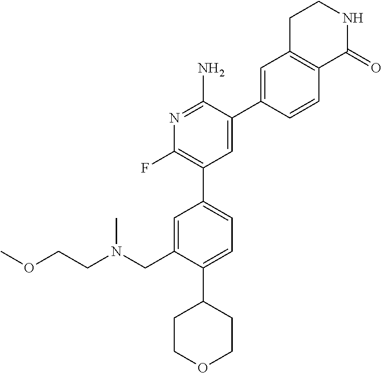 [SMILES: COCCN(C)Cc1cc(-c2cc(-c3ccc4c(c3)CCNC4=O)c(N)nc2F)ccc1C1CCOCC1] | A | |
| I-574 | 6-(2-amino-6-fluoro-5-(3- (((2- methoxyethyl)(methyl)a- mino)methyl)-4- (tetrahydro-2H-pyran-4- yl)phenyl)pyridin-3-yl)-7- fluoro-3,4- dihydroisoquinolin- 1(2H)-one | 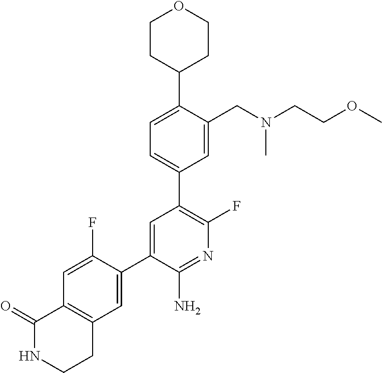 [SMILES: COCCN(C)Cc1cc(-c2cc(-c3cc4c(cc3F)C(=O)NCC4)c(N)nc2F)ccc1C1CCOCC1] | A | |
| I-575 | (R)-6-(2-amino-5-(3- ((dimethylamino)methyl)- 4-(2- methylmorpholino)phenyl)- 6-fluoropyridin-3-yl)- 3,4-dihydroisoquinolin- 1(2H)-one | 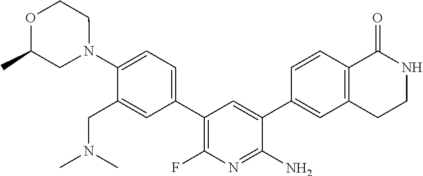 [SMILES: C[C@@H]1CN(c2ccc(-c3cc(-c4ccc5c(c4)CCNC5=O)c(N)nc3F)cc2CN(C)C)CCO1] | B | |
| I-576 | (R)-6-(2-amino-5-(3- ((dimethylamino)methyl)- 4-(2- methylmorpholino)phenyl)- 6-fluoropyridin-3-yl)-7- fluoro-3,4- dihydroisoquinolin- 1(2H)-one | 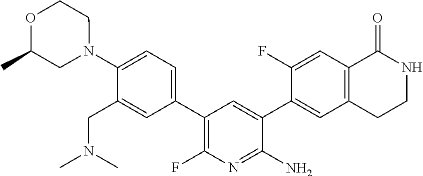 [SMILES: C[C@@H]1CN(c2ccc(-c3cc(-c4cc5c(cc4F)C(=O)NCC5)c(N)nc3F)cc2CN(C)C)CCO1] | A | |
| I-577 | 6-(2-amino-6-fluoro-5-(4- (4-isopropylpiperazin-1- yl)phenyl)pyridin-3-yl)-7- fluoro-4- methylisoquinolin-1(2H)- one | 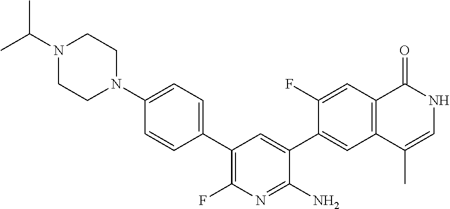 [SMILES: Cc1c[nH]c(=O)c2cc(F)c(-c3cc(-c4ccc(N5CCN(C(C)C)CC5)cc4)c(F)nc3N)cc12] | A | |
| I-578 | 6-(6′-amino-6-(azetidin- 1-ylmethyl)-2′-fluoro-5- morpholino-[2,3′- bipyridin]-5′-yl)-3,4- dihydroisoquinolin- 1(2H)-one | 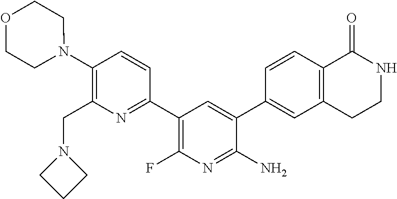 [SMILES: Nc1nc(F)c(-c2ccc(N3CCOCC3)c(CN3CCC3)n2)cc1-c1ccc2c(c1)CCNC2=O] | A | |
| I-579 | 6-(6′-amino-6-(azetidin- 1-ylmethyl)-2′-fluoro-5- morpholino-[2,3′- bipyridin]-5′-yl)-7-fluoro- 3,4-dihydroisoquinolin- 1(2H)-one | 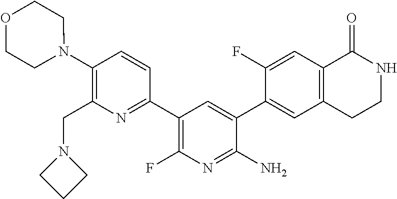 [SMILES: Nc1nc(F)c(-c2ccc(N3CCOCC3)c(CN3CCC3)n2)cc1-c1cc2c(cc1F)C(=O)NCC2] | A | |
| I-580 | 6-(6′-amino-6- ((ethyl(methyl)amino)meth- yl)-2′-fluoro-5- morpholino-[2,3′- bipyridin]-5′-yl)-3,4- dihydroisoquinolin- 1(2H)-one | 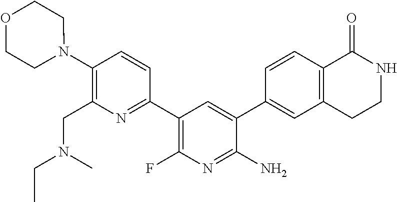 [SMILES: CCN(C)Cc1nc(-c2cc(-c3ccc4c(c3)CCNC4=O)c(N)nc2F)ccc1N1CCOCC1] | A | D |
| I-581 | 6-(6′-amino-6- ((ethyl(methyl)amino)meth- yl)-2′-fluoro-5- morpholino-[2,3′- bipyridin]-5′-yl)-7-fluoro- 3,4-dihydroisoquinolin- 1(2H)-one | 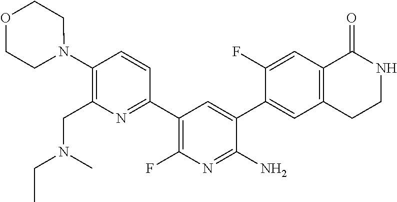 [SMILES: CCN(C)Cc1nc(-c2cc(-c3cc4c(cc3F)C(=O)NCC4)c(N)nc2F)ccc1N1CCOCC1] | A | |
| I-582 | 6-(6′-amino-2′-fluoro-6- (((2- methoxyethyl)(methyl)a- mino)methyl)-5- morpholino-[2,3′- bipyridin]-5′-yl)-3,4- dihydroisoquinolin- 1(2H)-one | 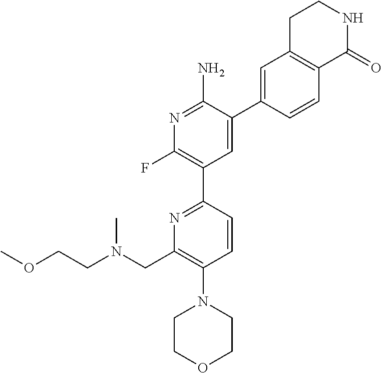 [SMILES: COCCN(C)Cc1nc(-c2cc(-c3ccc4c(c3)CCNC4=O)c(N)nc2F)ccc1N1CCOCC1] | A | |
| I-583 | 6-(6′-amino-2′-fluoro-6- (((2- methoxyethyl)(methyl)a- mino)methyl)-5- morpholino-[2,3′- bipyridin]-5′-yl)-7-fluoro- 3,4-dihydroisoquinolin- 1(2H)-one | 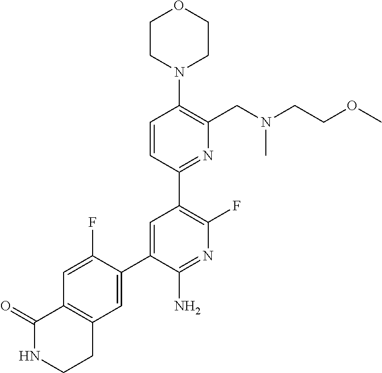 [SMILES: COCCN(C)Cc1nc(-c2cc(-c3cc4c(cc3F)C(=O)NCC4)c(N)nc2F)ccc1N1CCOCC1] | A | |
| I-584 | (S)-6-(6′-amino-2′-fluoro- 6-((3-methoxypyrrolidin- 1-yl)methyl)-5- morpholino-[2,3′- bipyridin]-5′-yl)-3,4- dihydroisoquinolin- 1(2H)-one | 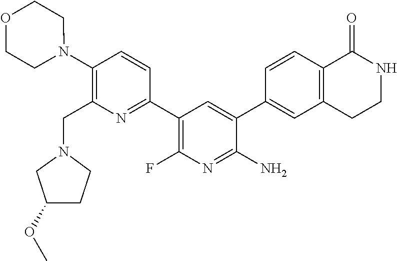 [SMILES: CO[C@H]1CCN(Cc2nc(-c3cc(-c4ccc5c(c4)CCNC5=O)c(N)nc3F)ccc2N2CCOCC2)C1] | A | |
| I-585 | (S)-6-(6′-amino-2′-fluroo- 6-((3-methoxypyrrolidin- 1-yl)methyl)-5- morpholino-[2,3′- bipyridin]-5′-yl)-7-fluoro- 3,4-dihydroisoquinolin- 1(2H)-one | 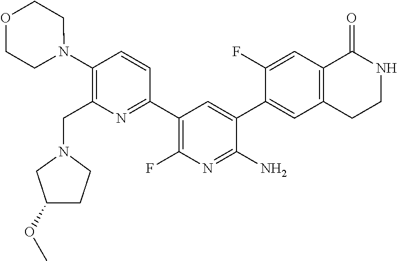 [SMILES: CO[C@H]1CCN(Cc2nc(-c3cc(-c4cc5c(cc4F)C(=O)NCC5)c(N)nc3F)ccc2N2CCOCC2)C1] | A | |
| I-586 | 6-(2-amino-6-fluoro-5-(4- (tetrahydro-2H-pyran-4- yl)phenyl)pyridin-3-yl)-4- methylisoquinolin-1(2H)- one | 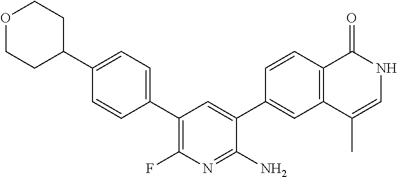 [SMILES: Cc1c[nH]c(=O)c2ccc(-c3cc(-c4ccc(C5CCOCC5)cc4)c(F)nc3N)cc12] | B | |
| I-587 | 6-(2-amino-6-fluoro-5-(4- (4-isopropylpiperazin-1- yl)phenyl)pyridin-3-yl)-7- fluoro-3- methylisoquinolin-1(2H)- one | 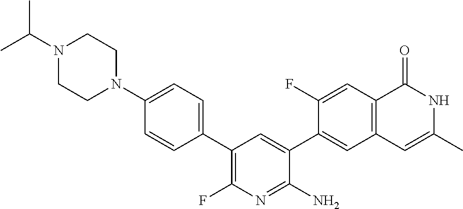 [SMILES: Cc1cc2cc(-c3cc(-c4ccc(N5CCN(C(C)C)CC5)cc4)c(F)nc3N)c(F)cc2c(=O)[nH]1] | B | |
| I-588 | 6-(6′-amino-6- ((dimethylamino)methyl)- 2′-fluoro-5-morpholino- [2,3′-bipyridin]-5′-yl)-4- methylisoquinolin-1(2H)- one | 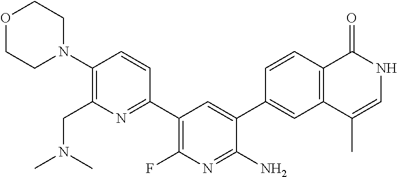 [SMILES: Cc1c[nH]c(=O)c2ccc(-c3cc(-c4ccc(N5CCOCC5)c(CN(C)C)n4)c(F)nc3N)cc12] | A | |
| I-589 | 6-(6′-amino-6- ((dimethylamino)methyl)- 2′-fluoro-5-morpholino- [2,3′-bipyridin]-5′-yl)-4- fluoroisoquinolin-1(2H)- one | 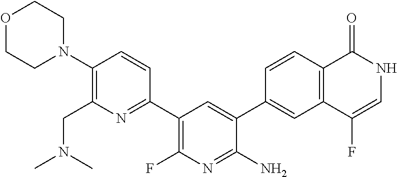 [SMILES: CN(C)Cc1nc(-c2cc(-c3ccc4c(=O)[nH]cc(F)c4c3)c(N)nc2F)ccc1N1CCOCC1] | A | |
| I-590 | tert-butyl 4-(6′-amino-2′- fluoro-5′-(1-oxo-1,2,3,4- tetrahydroisoquinolin-6- yl)-[2,3′-bipyridin]-5-yl) piperazine-1-carboxylate | 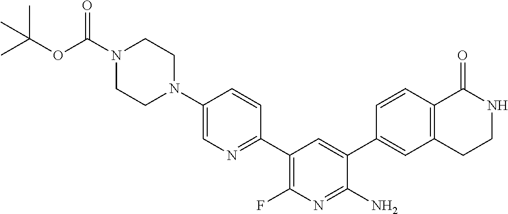 [SMILES: CC(C)(C)OC(=O)N1CCN(c2ccc(-c3cc(-c4ccc5c(c4)CCNC5=O)c(N)nc3F)nc2)CC1] | A | |
| I-591 | 6-(5-(4-acetylpiperazin- 1-yl)-6′-amino-2′-fluoro- [2,3′-bipyridin]-5′-yl)-3,4- dihydroisoquinolin- 1(2H)-one | 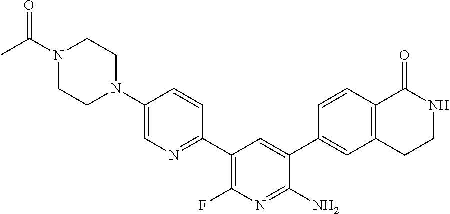 [SMILES: CC(=O)N1CCN(c2ccc(-c3cc(-c4ccc5c(c4)CCNC5=O)c(N)nc3F)nc2)CC1] | A | D |
| I-592 | 6-(5-(4-acetylpiperazin- 1-yl)-6′-amino-2′-fluoro- [2,3′-bipyridin]-5′-yl)-7- fluoro-3,4- dihydroisoquinolin- 1(2H)-one | 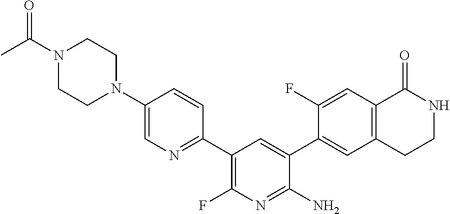 [SMILES: CC(=O)N1CCN(c2ccc(-c3cc(-c4cc5c(cc4F)C(=O)NCC5)c(N)nc3F)nc2)CC1] | A | |
| I-593 | 6-(6′-amino-5-(4-(4,4- difluorobutyl)piperazin-1- yl)-2′-fluoro-[2,3′- bipyridin]-5′-yl)-3,4- dihydroisoquinolin- 1(2H)-one | 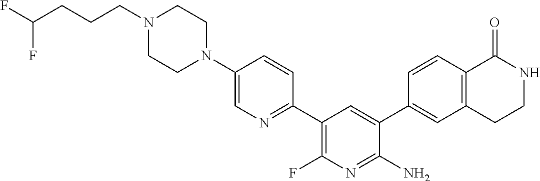 [SMILES: Nc1nc(F)c(-c2ccc(N3CCN(CCCC(F)F)CC3)cn2)cc1-c1ccc2c(c1)CCNC2=O] | A | |
| I-594 | 6-(2-amino-5-(3- ((dimethylamino)methyl) phenyl)-6-fluoropyridin- 3-yl)-3,4- dihydroisoquinolin- 1(2H)-one | 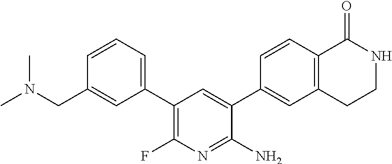 [SMILES: CN(C)Cc1cccc(-c2cc(-c3ccc4c(c3)CCNC4=O)c(N)nc2F)c1] | B | |
| I-595 | 7-(2-amino-5-(3- ((dimethylamino)methyl)- 4-((2R,6S)-2,6- dimethylmorpholino)phe- nyl)-6-fluoropyridin-3-yl)- 2-methylquinazolin- 4(3H)-one | 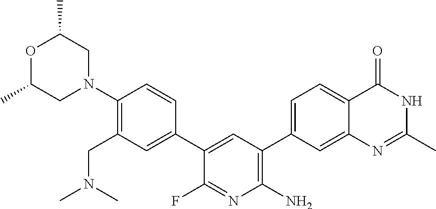 | B | |
| I-596 | 6-(2-amino-5-(3- ((dimethylamino)methyl)- 4-((2R,6S)-2,6- dimethylmorpholino)phe- nyl)-6-fluoropyridin-3-yl)- 8-fluoro-3,4- dihydroisoquinolin- 1(2H)-one | 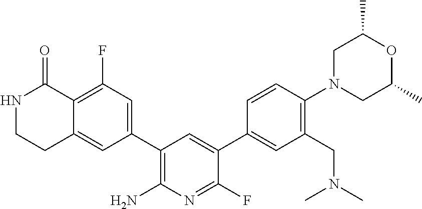 [SMILES: C[C@@H]1CN(c2ccc(-c3cc(-c4cc(F)c5c(c4)CCNC5=O)c(N)nc3F)cc2CN(C)C)C[C@H](C)O1] | B | |
| I-597 | 6-(2-amino-5-(3- ((dimethylamino)methyl)- 4-((2R,6S)-2,6- dimethylmorpholino)phe- nyl)-6-fluoropyridin-3-yl)- 7-fluoro-3,4- dihydroisoquinolin- 1(2H)-one | 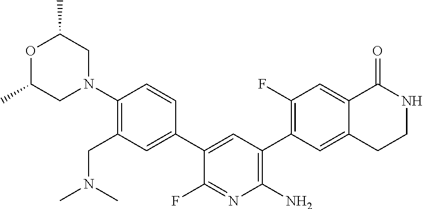 [SMILES: C[C@@H]1CN(c2ccc(-c3cc(-c4cc5c(cc4F)C(=O)NCC5)c(N)nc3F)cc2CN(C)C)C[C@H](C)O1] | B | |
| I-598 | (S)-6-(2-amino-5-(3- (azetidin-1-ylmethyl)-4- (2- methylmorpholino)phenyl)- 6-fluoropyridin-3-yl)- 3,4-dihydroisoquinolin- 1(2H)-one | 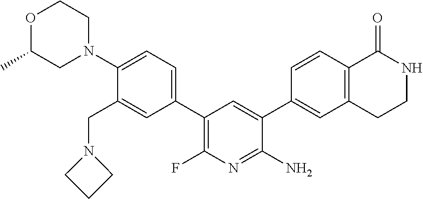 [SMILES: C[C@H]1CN(c2ccc(-c3cc(-c4ccc5c(c4)CCNC5=O)c(N)nc3F)cc2CN2CCC2)CCO1] | B | |
| I-599 | (S)-6-(2-amino-5-(3- (azetidin-1-ylmethyl)-4- (2- methylmorpholino)phenyl)- 6-fluoropyridin-3-yl)-7- fluoro-3,4- dihydroisoquinolin- 1(2H)-one | 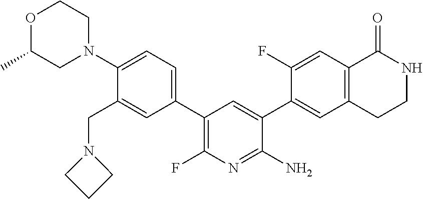 [SMILES: C[C@H]1CN(c2ccc(-c3cc(-c4cc5c(cc4F)C(=O)NCC5)c(N)nc3F)cc2CN2CCC2)CCO1] | B | |
| I-600 | 6-(6′-amino-6- ((dimethylamino)methyl)- 2′-fluoro-5-(tetrahydro- 2H-pyran-4-yl)-[2,3′- bipyridin]-5′-yl)-3,4- dihydroisoquinolin- 1(2H)-one | 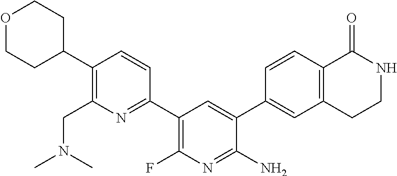 [SMILES: CN(C)Cc1nc(-c2cc(-c3ccc4c(c3)CCNC4=O)c(N)nc2F)ccc1C1CCOCC1] | A | |
| I-601 | 6-(6′-amino-6- ((dimethylamino)methyl)- 2′-fluoro-5-(tetrahydro- 2H-pyran-4-yl)-[2,3′- bipyridin]-5′-yl)-7-fluoro- 3,4-dihydroisoquinolin- 1(2H)-one | 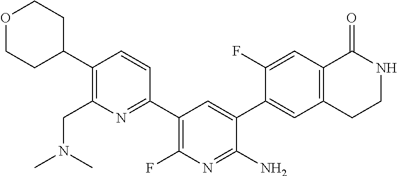 [SMILES: CN(C)Cc1nc(-c2cc(-c3cc4c(cc3F)C(=O)NCC4)c(N)nc2F)ccc1C1CCOCC1] | A | |
| I-602 | 6-(6′-amino-6-(azetidin- 1-ylmethyl)-2′-fluoro-5- (tetrahydro-2H-pyran-4- yl)-[2,3′-bipyridin]-5′-yl)- 7-fluoro-3,4- dihydroisoquinolin- 1(2H)-one | 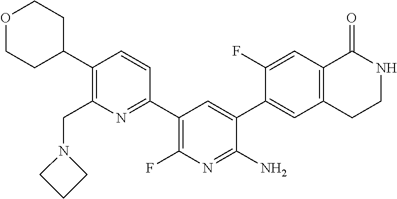 [SMILES: Nc1nc(F)c(-c2ccc(C3CCOCC3)c(CN3CCC3)n2)cc1-c1cc2c(cc1F)C(=O)NCC2] | A | |
| I-603 | 6-(6′-amino-6- ((ethyl(methyl)amino)meth- yl)-2′-fluoro-5- (tetrahydro-2H-pyran-4- yl)-[2,3′-bipyridin]-5′-yl)- 3,4-dihydroisoquinolin- 1(2H)-one | 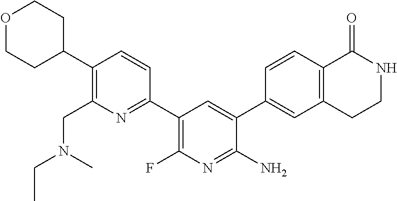 [SMILES: CCN(C)Cc1nc(-c2cc(-c3ccc4c(c3)CCNC4=O)c(N)nc2F)ccc1C1CCOCC1] | A | |
| I-604 | 6-(6′-amino-6- ((ethyl(methyl)amino)meth- yl)-2′-fluoro-5- (tetrahydro-2H-pyran-4- yl)-[2,3′-bipyridin]-5′-yl)- 7-fluoro-3,4- dihydroisoquinolin- 1(2H)-one | 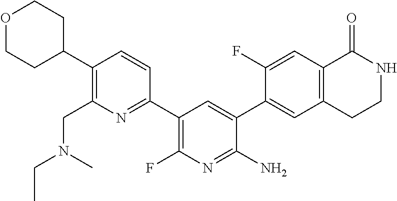 [SMILES: CCN(C)Cc1nc(-c2cc(-c3cc4c(cc3F)C(=O)NCC4)c(N)nc2F)ccc1C1CCOCC1] | A | |
| I-605 | 6-(6′-amino-6-(azetidin- 1-ylmethyl)-2′-fluoro-5- (tetrahydro-2H-pyran-4- yl)-[2,3′-bipyridin]-5′-yl)- 3,4-dihydroisoquinolin- 1(2H)-one | 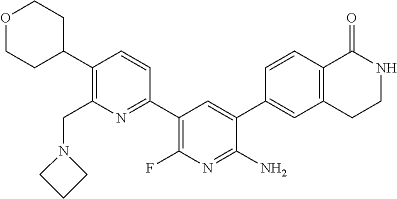 [SMILES: Nc1nc(F)c(-c2ccc(C3CCOCC3)c(CN3CCC3)n2)cc1-c1ccc2c(c1)CCNC2=O] | A | |
| I-606 | 6-(2-amino-5-(3- ((dimethylamino)methyl)- 2-fluoro-4- morpholinophenyl)-6- fluoropyridin-3-yl)-3,4- dihydroisoquinolin- 1(2H)-one | 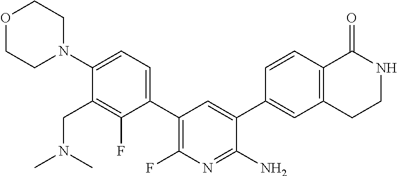 [SMILES: CN(C)Cc1c(N2CCOCC2)ccc(-c2cc(-c3ccc4c(c3)CCNC4=O)c(N)nc2F)c1F] | B | |
| I-607 | 6-(2-amino-5-(3- ((dimethylamino)methyl)- 2-fluoro-4- morpholinophenyl)-6- fluoropyridin-3-yl)-7- fluoro-3,4- dihydroisoquinolin- 1(2H)-one | 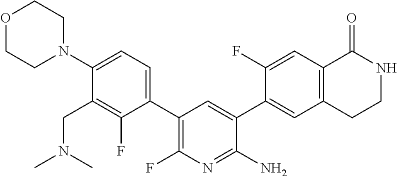 [SMILES: CN(C)Cc1c(N2CCOCC2)ccc(-c2cc(-c3cc4c(cc3F)C(=O)NCC4)c(N)nc2F)c1F] | B | |
| I-608 | 6-(2-amino-5-(3- (azetidin-1-ylmethyl)-2- fluoro-4- morpholinophenyl)-6- fluoropyridin-3-yl)-7- fluoro-3,4- dihydroisoquinolin- 1(2H)-one | 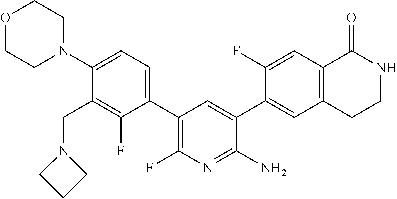 [SMILES: Nc1nc(F)c(-c2ccc(N3CCOCC3)c(CN3CCC3)c2F)cc1-c1cc2c(cc1F)C(=O)NCC2] | B | |
| I-60 | 6-(2-amino-5-(3- (azetidin-1-ylmethyl)-2- fluoro-4- morpholinophenyl)-6- fluoropyridin-3-yl)-3,4- dihydroisoquinolin- 1(2H)-one | 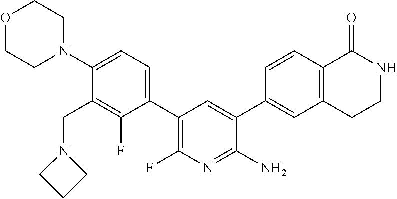 [SMILES: Nc1nc(F)c(-c2ccc(N3CCOCC3)c(CN3CCC3)c2F)cc1-c1ccc2c(c1)CCNC2=O] | B | D |
| I-610 | 6-(2-amino-5-(3- ((ethyl(methyl)amino)meth- yl)-2-fluoro-4- morpholinophenyl)-6- fluoropyridin-3-yl)-3,4- dihydroisoquinolin- 1(2H)-one | 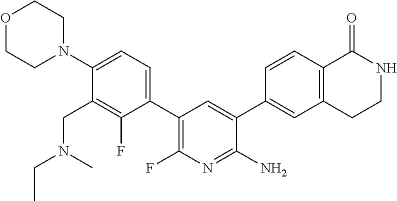 [SMILES: CCN(C)Cc1c(N2CCOCC2)ccc(-c2cc(-c3ccc4c(c3)CCNC4=O)c(N)nc2F)c1F] | B | |
| I-611 | 6-(2-amino-5-(3- ((ethyl(methyl)amino)meth- yl)-2-fluoro-4- morpholinophenyl)-6- fluoropyridin-3-yl)-7- fluoro-3,4- dihydroisoquinolin- 1(2H)-one | 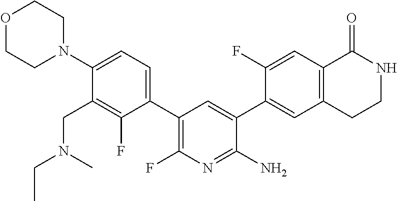 [SMILES: CCN(C)Cc1c(N2CCOCC2)ccc(-c2cc(-c3cc4c(cc3F)C(=O)NCC4)c(N)nc2F)c1F] | B | |
| I-612 | 6-(6-amino-5′- ((dimethylamino)methyl)- 2-fluoro-6′-morpholino- [3,3′-bipyridin]-5-yl)-3,4- dihydroisoquinolin- 1(2H)-one | 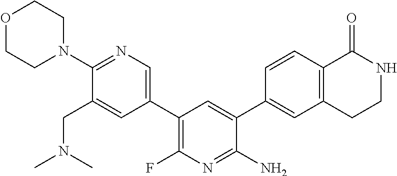 [SMILES: CN(C)Cc1cc(-c2cc(-c3ccc4c(c3)CCNC4=O)c(N)nc2F)cnc1N1CCOCC1] | B | |
| I-613 | 6-(6′-amino-4- ((dimethylamino)methyl)- 2′-fluoro-5-morpholino- [2,3′-bipyridin]-5′-yl)-3,4- dihydroisoquinolin- 1(2H)-one | 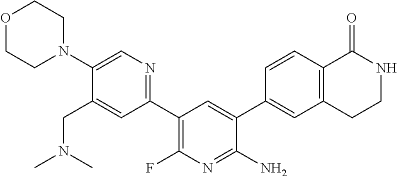 [SMILES: CN(C)Cc1cc(-c2cc(-c3ccc4c(c3)CCNC4=O)c(N)nc2F)ncc1N1CCOCC1] | B | |
| I-614 | 6-(2-amino-6-fluoro-5-(3- ((isopropyl(methyl)amino) methyl)-4- morpholinophenyl)pyridin- 3-yl)-3,4- dihydroisoquinolin- 1(2H)-one | 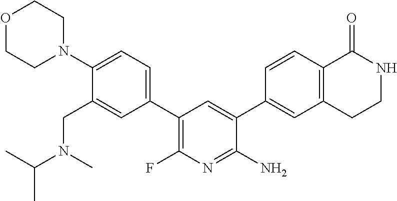 [SMILES: CC(C)N(C)Cc1cc(-c2cc(-c3ccc4c(c3)CCNC4=O)c(N)nc2F)ccc1N1CCOCC1] | B | |
| I-615 | 6-(2-amino-5-(3- ((diethylamino)methyl)-4- morpholinophenyl)-6- fluoropyridin-3-yl)-3,4- dihydroisoquinolin- 1(2H)-one | 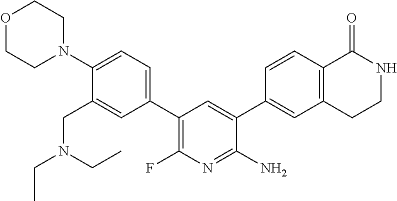 [SMILES: CCN(CC)Cc1cc(-c2cc(-c3ccc4c(c3)CCNC4=O)c(N)nc2F)ccc1N1CCOCC1] | A | |
| I-16 | 6-(2-amino-6-fluoro-5-(3- ((methyl((tetrahydrofuran- 2- yl)methyl)amino)methyl)- 4- morpholinophenyl)pyridin- 3-yl)-3,4- dihydroisoquinolin- 1(2H)-one | 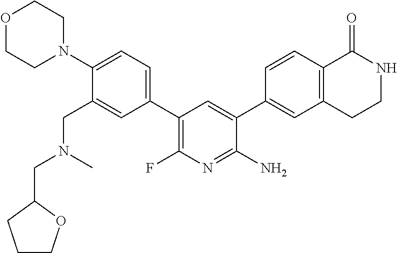 [SMILES: CN(Cc1cc(-c2cc(-c3ccc4c(c3)CCNC4=O)c(N)nc2F)ccc1N1CCOCC1)CC1CCCO1] | A | |
| I-617 | 6-(2-amino-5-(3- ((dimethylamino)methyl)- 4-fluorophenyl)-6- fluoropyridin-3-yl)-3,4- dihydroisoquinolin- 1(2H)-one | 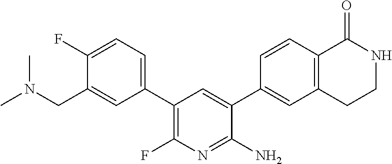 [SMILES: CN(C)Cc1cc(-c2cc(-c3ccc4c(c3)CCNC4=O)c(N)nc2F)ccc1F] | B | |
| I-618 | 6-(2-amino-5-(3- ((dimethylamino)methyl)- 4-fluorophenyl)-6- fluoropyridin-3-yl)-7- fluoro-3,4- dihydroisoquinolin- 1(2H)-one | 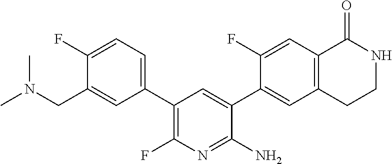 [SMILES: CN(C)Cc1cc(-c2cc(-c3cc4c(cc3F)C(=O)NCC4)c(N)nc2F)ccc1F] | B | |
| I-619 | 6-(2-amino-6-fluoro-5-(3- ((methyl((tetrahydrofuran- 2- yl)methyl)amino)methyl)- 4- morpholinophenyl)pyridin- 3-yl)-7-fluoro-3,4- dihydroisoquinolin- 1(2H)-one | 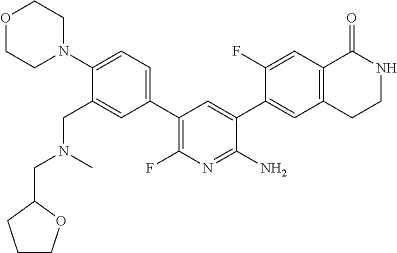 [SMILES: CN(Cc1cc(-c2cc(-c3cc4c(cc3F)C(=O)NCC4)c(N)nc2F)ccc1N1CCOCC1)CC1CCCO1] | B | |
| I-620 | 6-(2-amino-5-(3- ((dimethylamino)methyl)- 4-(tetrahydrofuran-3- yl)phenyl)-6- fluoropyridin-3-yl)-3,4- dihydroisoquinolin- 1(2H)-one | 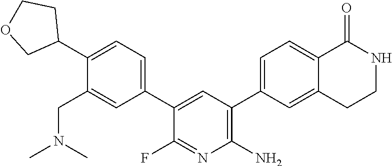 [SMILES: CN(C)Cc1cc(-c2cc(-c3ccc4c(c3)CCNC4=O)c(N)nc2F)ccc1C1CCOC1] | A | |
| I-621 | 6-(2-amino-5-(3- ((dimethylamino)methyl)- 4-(tetrahydrofuran-3- yl)phenyl)-6- fluoropyridin-3-yl)-7- fluoro-3,4- dihydroisoquinolin- 1(2H)-one | 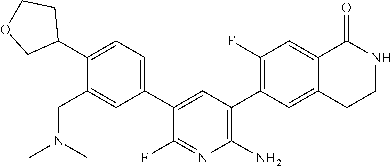 [SMILES: CN(C)Cc1cc(-c2cc(-c3cc4c(cc3F)C(=O)NCC4)c(N)nc2F)ccc1C1CCOC1] | B | |
| I-622 | 6-(2-amino-5-(5- ((dimethylamino)methyl)- 2-fluoro-4- morpholinophenyl)-6- fluoropyridin-3-yl)-3,4- dihydroisoquinolin- 1(2H)-one | 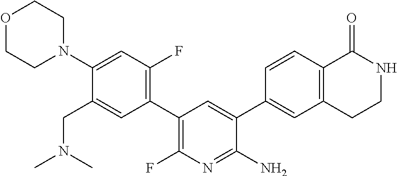 [SMILES: CN(C)Cc1cc(-c2cc(-c3ccc4c(c3)CCNC4=O)c(N)nc2F)c(F)cc1N1CCOCC1] | B | |
| I-623 | 6-(2-amino-5-(5- ((dimethylamino)methyl)- 2,3-difluoro-4- morpholinophenyl)-6- fluoropyridin-3-yl)-3,4- dihydroisoquinolin- 1(2H)-one | 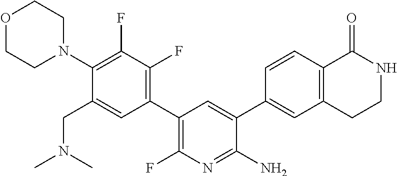 [SMILES: CN(C)Cc1cc(-c2cc(-c3ccc4c(c3)CCNC4=O)c(N)nc2F)c(F)c(F)c1N1CCOCC1] | B | |
| I-624 | 6-(2-amino-5-(3-(1- (dimethylamino)ethyl)-4- hydroxyphenyl)-6- fluoropyridin-3-yl)-3,4- dihydroisoquinolin- 1(2H)-one | 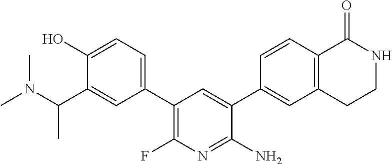 [SMILES: CC(c1cc(-c2cc(-c3ccc4c(c3)CCNC4=O)c(N)nc2F)ccc1O)N(C)C] | B | |
| I-625 | 6-(2-amino-5-(3-(2- aminopropan-2-yl)-4- fluorophenyl)-6- fluoropyridin-3-yl)-3,4- dihydroisoquinolin- 1(2H)-one | 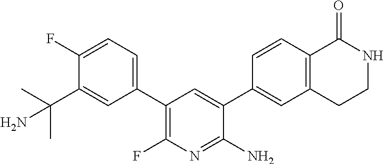 [SMILES: CC(C)(N)c1cc(-c2cc(-c3ccc4c(c3)CCNC4=O)c(N)nc2F)ccc1F] | C | |
| I-626 | 6-(2-amino-5-(3- ((dimethylamino)methyl)- 4-fluorophenyl)-6- fluoropyridin-3-yl)-4- methylisoquinolin-1(2H)- one | 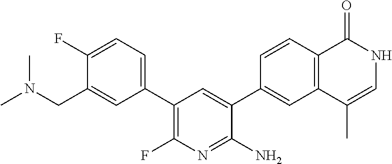 [SMILES: Cc1c[nH]c(=O)c2ccc(-c3cc(-c4ccc(F)c(CN(C)C)c4)c(F)nc3N)cc12] | C | |
| I-627 | 6-(2-amino-5-(3- ((dimethylamino)methyl) phenyl)-6-fluoropyridin- 3-yl)-4- methylisoquinolin-1(2H)- one | 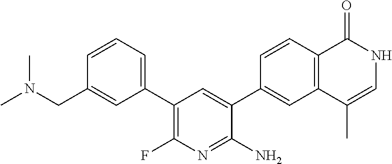 [SMILES: Cc1c[nH]c(=O)c2ccc(-c3cc(-c4cccc(CN(C)C)c4)c(F)nc3N)cc12] | C | |
| I-628 | (R)-6-(2-amino-5-(4- (dimethylamino)chroman- 6-yl)-6-fluoropyridin-3- yl)-4-methylisoquinolin- 1(2H)-one | 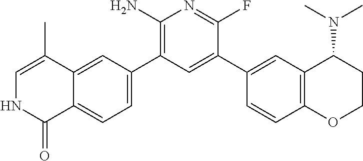 [SMILES: Cc1c[nH]c(=O)c2ccc(-c3cc(-c4ccc5c(c4)[C@H](N(C)C)CCO5)c(F)nc3N)cc12] | B | |
| I-629 | (S)-6-(2-amino-5-(3- (dimethylamino)-2,3- dihydrobenzofuran-5-yl)- 6-fluoropyridin-3-yl)-4- methylisoquinolin-1(2H)- one | 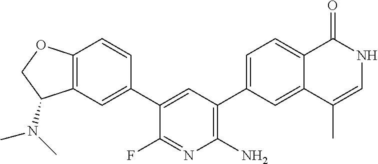 [SMILES: Cc1c[nH]c(=O)c2ccc(-c3cc(-c4ccc5c(c4)[C@H](N(C)C)CO5)c(F)nc3N)cc12] | C | |
| I-630 | (S)-6-(2-amino-5-(3- (dimethylamino)-2,3- dihydrobenzofuran-5-yl)- 6-fluoropyridin-3-yl)-4- fluoroisoquinolin-1(2H)- one | 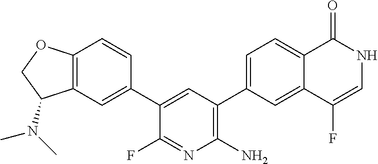 [SMILES: CN(C)[C@@H]1COc2ccc(-c3cc(-c4ccc5c(=O)[nH]cc(F)c5c4)c(N)nc3F)cc21] | B | |
| I-631 | (R)-6-(2-amino-5-(4- (dimethylamino)chroman- 6-yl)-6-fluoropyridin-3- yl)-4-fluoroisoquinolin- 1(2H)-one | 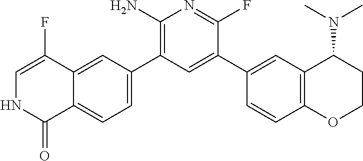 [SMILES: CN(C)[C@@H]1CCOc2ccc(-c3cc(-c4ccc5c(=O)[nH]cc(F)c5c4)c(N)nc3F)cc21] | B | |
| I-632 | 5-(6-amino-2-fluoro-5-(1- oxo-1,2,3,4- tetrahydroisoquinolin-6- yl)pyridin-3-yl)-2- methoxybenzonitrile | 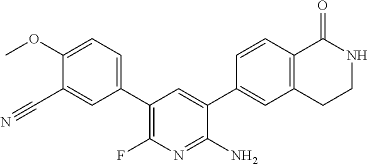 [SMILES: COc1ccc(-c2cc(-c3ccc4c(c3)CCNC4=O)c(N)nc2F)cc1C#N] | B | |
| I-633 | 6-(2-amino-5-(3- ((ethyl(methyl)amino)meth- yl)-4-methoxyphenyl)-6- fluoropyridin-3-yl)-4- fluoroisoquinolin-1(2H)- one | 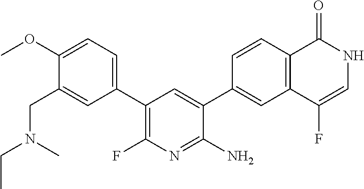 [SMILES: CCN(C)Cc1cc(-c2cc(-c3ccc4c(=O)[nH]cc(F)c4c3)c(N)nc2F)ccc1OC] | B | |
| I-634 | 6-(2-amino-5-(3- ((ethyl(methyl)amino)meth- yl)-4-methoxyphenyl)-6- fluoropyridin-3-yl)-4- methylisoquinolin-1(2H)- one | 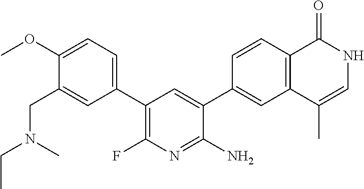 [SMILES: CCN(C)Cc1cc(-c2cc(-c3ccc4c(=O)[nH]cc(C)c4c3)c(N)nc2F)ccc1OC] | B | |
| I-635 | 8-(2-amino-5-(4-(4- (cyclopropylmethyl)piper- azin-1-yl)phenyl)-6- fluoropyridin-3-yl)-6- fluoro-3,4- dihydrobenzo[f][1,4]oxaze- pin-5(2H)-one | 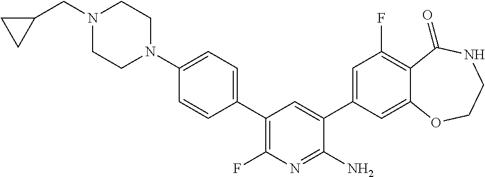 [SMILES: Nc1nc(F)c(-c2ccc(N3CCN(CC4CC4)CC3)cc2)cc1-c1cc(F)c2c(c1)OCCNC2=O] | A | |
| I-636 | 8-(2-amino-6-fluoro-5-(4- (4-isopropylpiperazin-1- yl)phenyl)pyridin-3-yl)-6- fluoro-3,4- dihydrobenzo[f][1,4]oxaze- pin-5(2H)-one | 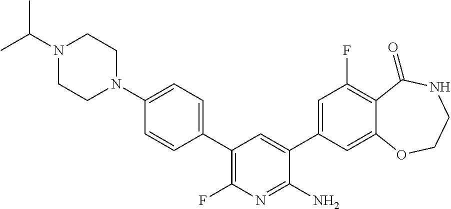 [SMILES: CC(C)N1CCN(c2ccc(-c3cc(-c4cc(F)c5c(c4)OCCNC5=O)c(N)nc3F)cc2)CC1] | A | |
| I-637 | 5-(2-amino-6-fluoro-5-(4- (4-isopropylpiperazin-1- yl)phenyl)pyridin-3-yl)-7- fluoroisoindolin-1-one | 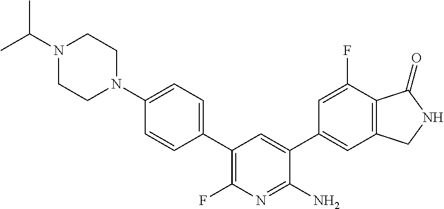 [SMILES: CC(C)N1CCN(c2ccc(-c3cc(-c4cc(F)c5c(c4)CNC5=O)c(N)nc3F)cc2)CC1] | B | |
| I-638 | 6-(2-amino-6-fluoro-5-(3- (3-((4-methyl-4H-1,2,4- triazol-3- yl)methyl)oxetan-3- yl)phenyl)pyridin-3-yl)- 3,4-dihydroisoquinolin- 1(2H)-one | 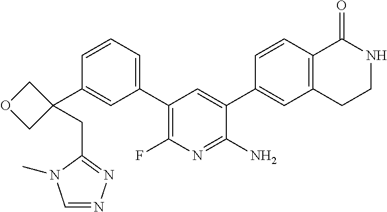 [SMILES: Cn1cnnc1CC1(c2cccc(-c3cc(-c4ccc5c(c4)CCNC5=O)c(N)nc3F)c2)COC1] | C | |
| I-639 | 6-((6-fluoro-5-(4-(4- isopropylpiperazin-1- yl)phenyl)pyridin-2- yl)amino)-3,4- dihydroisoquinolin- 1(2H)-one | 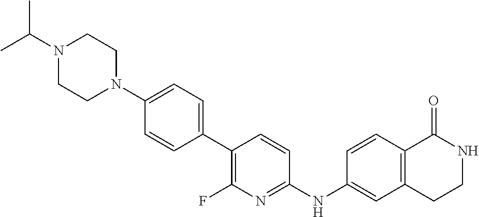 [SMILES: CC(C)N1CCN(c2ccc(-c3ccc(Nc4ccc5c(c4)CCNC5=O)nc3F)cc2)CC1] | D | |
| I-640 | 6-(2-amino-5-(4-(4-((2,2- difluorocyclopropyl)meth- yl)piperazin-1-yl)phenyl)- 6-fluoropyridin-3-yl)-3,4- dihydroisoquinolin- 1(2H)-one | 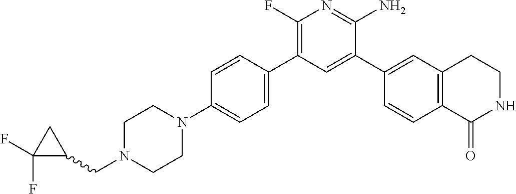 [SMILES: Nc1nc(F)c(-c2ccc(N3CCN(CC4CC4(F)F)CC3)cc2)cc1-c1ccc2c(c1)CCNC2=O] | A | |
| I-641 | 6-(2-amino-5-(4-(4-(4,4- difluorobutyl)piperazin-1- yl)phenyl)-6- fluoropyridin-3-yl)-3,4- dihydroisoquinolin- 1(2H)-one | 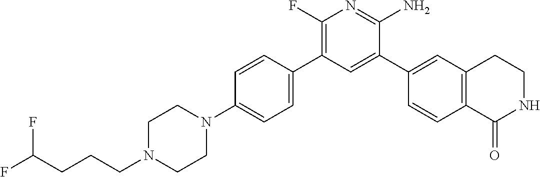 [SMILES: Nc1nc(F)c(-c2ccc(N3CCN(CCCC(F)F)CC3)cc2)cc1-c1ccc2c(c1)CCNC2=O] | A | |
| I-642 | (S)-6-(2-amino-6-fluoro- 5-(3-(1-methylpyrrolidin- 2-yl)-4-(tetrahydro-2H- pyran-4- yl)phenyl)pyridin-3-yl)- 3,4-dihydroisoquinolin- 1(2H)-on | 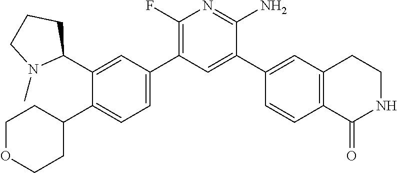 [SMILES: CN1CCC[C@H]1c1cc(-c2cc(-c3ccc4c(c3)CCNC4=O)c(N)nc2F)ccc1C1CCOCC1] | B | |
| I-643 | (S)-7-(2-amino-6-fluoro- 5-(3-(pyrrolidin-2-yl)-4- (tetrahydro-2H-pyran-4- yl)phenyl)pyridin-3-yl)-2- methylquinazolin-4(3H)- one | 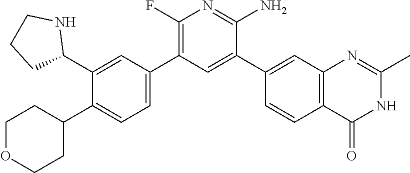 [SMILES: Cc1nc2cc(-c3cc(-c4ccc(C5CCOCC5)c([C@@H]5CCCN5)c4)c(F)nc3N)ccc2c(=O)[nH]1] | B | |
| I-644 | 6-(2-amino-5-(4-(4-(2- (2,2- difluorocyclopropyl)ethyl) piperazin-1-yl)phenyl)-6- fluoropyridin-3-yl)-3,4- dihydroisoquinolin- 1(2H)-one | 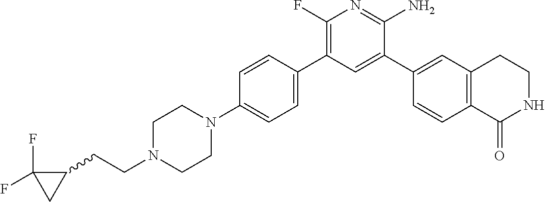 [SMILES: Nc1nc(F)c(-c2ccc(N3CCN(CCC4CC4(F)F)CC3)cc2)cc1-c1ccc2c(c1)CCNC2=O] | A | |
| I-645 | 6-(2-amino-5-(3- ((dimethylamino)methyl)- 4-(tetrahydro-2H-pyran- 4-yl)phenyl)-6- fluoropyridin-3-yl)-3,4- dihydroisoquinolin- 1(2H)-one | 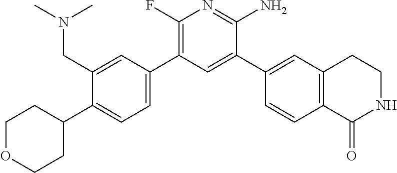 [SMILES: CN(C)Cc1cc(-c2cc(-c3ccc4c(c3)CCNC4=O)c(N)nc2F)ccc1C1CCOCC1] | A | |
| I-646 | (S)-6-(2-amino-6-fluoro- 5-(3-(pyrrolidin-2- yl)phenyl)pyridin-3-yl)- 3,4-dihydroisoquinolin- 1(2H)-one | 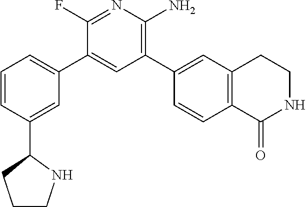 [SMILES: Nc1nc(F)c(-c2cccc([C@@H]3CCCN3)c2)cc1-c1ccc2c(c1)CCNC2=O] | A | |

A: 0.1-10 nM;
B: 11-100 nM;
C: 101-1000 nM;
D > 1000 nM

TABLE 3
Comparative Compounds

| Comparative | | |
|---|---|---|
| Compound I.D. | IUPAC chemical name | Structure |
| Comparator 1 | 6-(2-amino-5-(4-(piperidin-4- yl)phenyl)pyridin-3-yl)-3,4- dihydroisoquinolin-1(2H)-one | 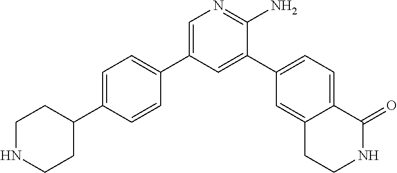 [SMILES: Nc1ncc(-c2ccc(C3CCNCC3)cc2)cc1-c1ccc2c(c1)CCNC2=O] |
| Comparator 2 | 6-(2-amino-5-(1-(tetrahydro-2H- pyran-4-yl)-1H-pyrazol-4- yl)pyridin-3-yl)-3,4- dihydroisoquinolin-1(2H)-one | 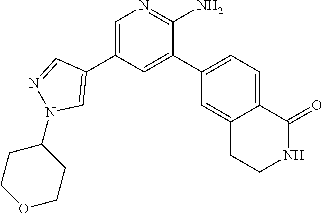 [SMILES: Nc1ncc(-c2cnn(C3CCOCC3)c2)cc1-c1ccc2c(c1)CCNC2=O] |
| Comparator 3 | 6-(2-amino-5-(4-(pyrrolidin-1- yl)phenyl)pyridin-3-yl)-3,4- dihydroisoquinolin-1(2H)-one | 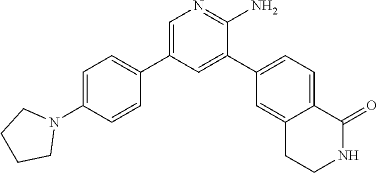 [SMILES: Nc1ncc(-c2ccc(N3CCCC3)cc2)cc1-c1ccc2c(c1)CCNC2=O] |
| Comparator 4 | 5-(2-amino-5-(4-(piperidin-1- yl)phenyl)pyridin-3-yl)isoindolin- 1-one | 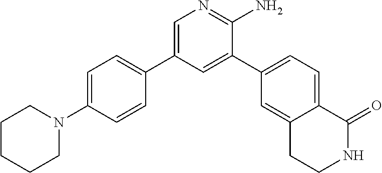 [SMILES: Nc1ncc(-c2ccc(N3CCCCC3)cc2)cc1-c1ccc2c(c1)CCNC2=O] |
| Comparator 5 | 6-(2-amino-5-(4-((3aR,6aS)- tetrahydro-1H-furo[3,4-c]pyrrol- 5(3H)-yl)phenyl)pyridin-3-yl)-3,4- dihydroisoquinolin-1(2H)-one | 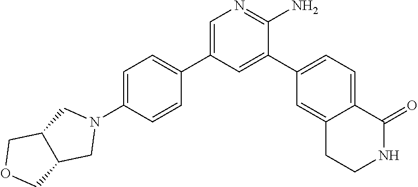 [SMILES: Nc1ncc(-c2ccc(N3C[C@H]4COC[C@H]4C3)cc2)cc1-c1ccc2c(c1)CCNC2=O] |
| Comparator 6 | 6-(2-amino-5-(4-((3aS,6aS)- hexahydro-5H-furo[2,3-c]pyrrol- 5-yl)phenyl)pyridin-3-yl)-3,4- dihydroisoquinolin-1(2H)-one | 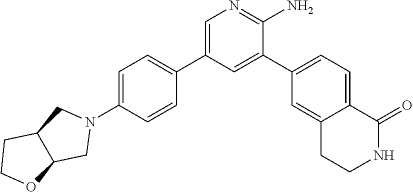 [SMILES: Nc1ncc(-c2ccc(N3C[C@@H]4CCO[C@@H]4C3)cc2)cc1-c1ccc2c(c1)CCNC2=O] |
| Comparator 7 | 6-(2-amino-5-(4-(piperazin-1-yl)-3- (trifluoromethyl)phenyl)pyridin-3-yl)-3,4- dihydroisoquinolin-1(2H)-one | 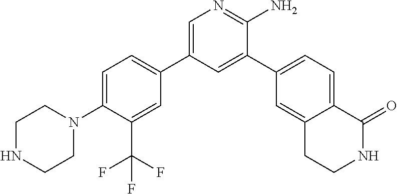 [SMILES: Nc1ncc(-c2ccc(N3CCNCC3)c(C(F)(F)F)c2)cc1-c1ccc2c(c1)CCNC2=O] |
| Comparator 8 | 6-(2-amino-5-(3,4-dihydro-2H- benzo[b][1,4]dioxepin-7- yl)pyridin-3-yl)-3,4- dihydroisoquinolin-1(2H)-one | 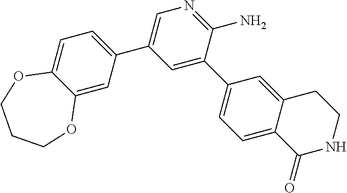 [SMILES: Nc1ncc(-c2ccc3c(c2)OCCCO3)cc1-c1ccc2c(c1)CCNC2=O] |
| Comparator 9 | 6-(2-amino-5-(4- (hexahydropyrrolo[1,2-a]pyrazin- 2(1H)-yl)phenyl)pyridin-3-yl)-3,4- dihydroisoquinolin-1(2H)-one | 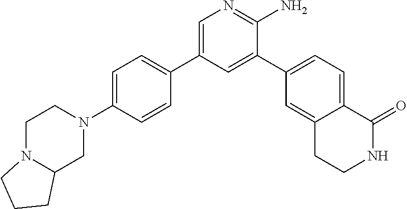 [SMILES: Nc1ncc(-c2ccc(N3CCN4CCCC4C3)cc2)cc1-c1ccc2c(c1)CCNC2=O] |
| Comparator 10 | 6-(2-amino-5-(4-(hexahydro-1H-pyrrolo[1,2- a][1,4]diazepin-2(3H)-yl)phenyl)pyridin-3-yl)- 3,4-dihydroisoquinolin-1(2H)-one | 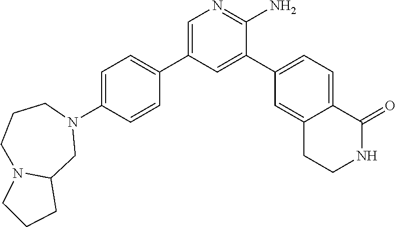 [SMILES: Nc1ncc(-c2ccc(N3CCCN4CCCC4C3)cc2)cc1-c1ccc2c(c1)CCNC2=O] |
| Comparator 11 | 6-(2-amino-5-(4-(piperidin-4- yl)phenyl)pyridin-3-yl)-7-fluoro- 3,4-dihydroisoquinolin-1(2H)-one | 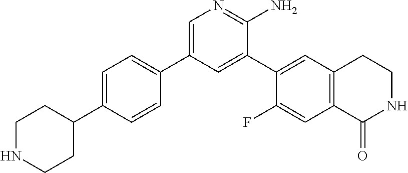 [SMILES: Nc1ncc(-c2ccc(C3CCNCC3)cc2)cc1-c1cc2c(cc1F)C(=O)NCC2] |

TABLE 4
LcK IC₅₀/HPK IC₅₀ selectivity

| Structure | Selectivity | Structure | Selectivity |
|---|---|---|---|
| 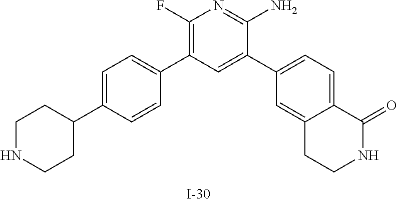 | 1603 | 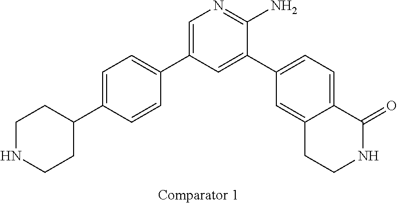 [SMILES: Nc1ncc(-c2ccc(C3CCNCC3)cc2)cc1-c1ccc2c(c1)CCNC2=O] | 45 |
| 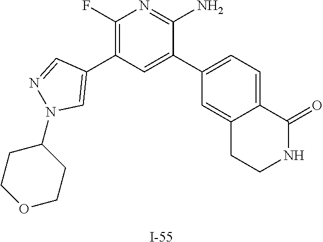 | 2000 | 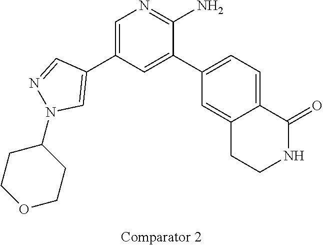 [SMILES: Nc1ncc(-c2cnn(C3CCOCC3)c2)cc1-c1ccc2c(c1)CCNC2=O] | 0.2 |
| 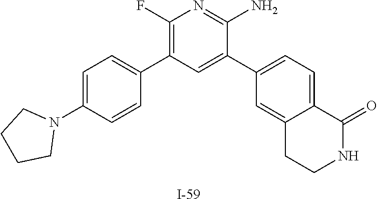 | 769 | 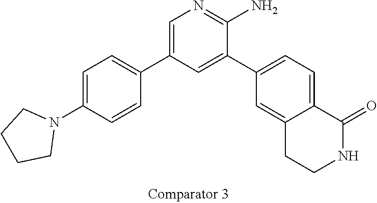 [SMILES: Nc1ncc(-c2ccc(N3CCCC3)cc2)cc1-c1ccc2c(c1)CCNC2=O] | 69 |
| 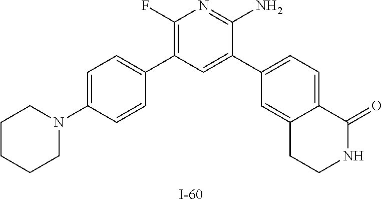 | 1250 | 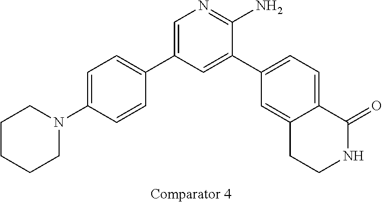 [SMILES: Nc1ncc(-c2ccc(N3CCCCC3)cc2)cc1-c1ccc2c(c1)CCNC2=O] | 79 |
| 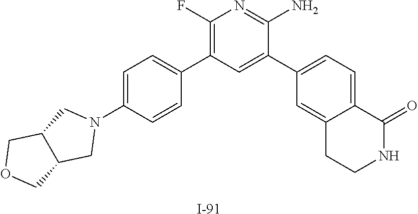 | 323 | 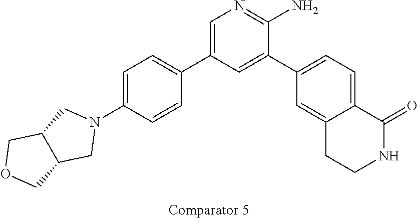 [SMILES: Nc1ncc(-c2ccc(N3C[C@H]4COC[C@H]4C3)cc2)cc1-c1ccc2c(c1)CCNC2=O] | 51 |
| 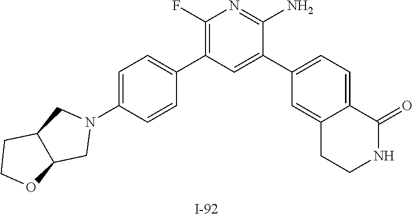 | 1429 | 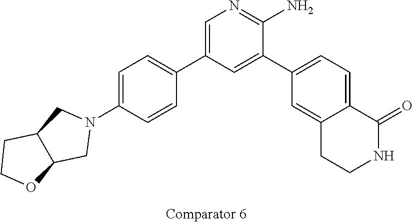 [SMILES: Nc1ncc(-c2ccc(N3C[C@@H]4CCO[C@@H]4C3)cc2)cc1-c1ccc2c(c1)CCNC2=O] | 34 |
| 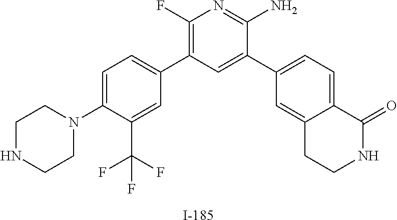 [SMILES: Nc1nc(F)c(-c2ccc(N3CCNCC3)c(C(F)(F)F)c2)cc1-c1ccc2c(c1)CCNC2=O ; "I-185"] | 2580 | 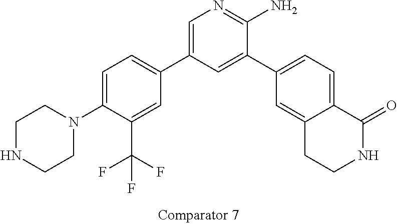 [SMILES: Nc1ncc(-c2ccc(N3CCNCC3)c(C(F)(F)F)c2)cc1-c1ccc2c(c1)CCNC2=O] | 145 |
| 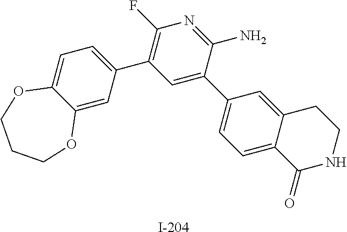 [SMILES: Nc1nc(F)c(-c2ccc3c(c2)OCCCO3)cc1-c1ccc2c(c1)CCNC2=O ; "I-204"] | 1429 | 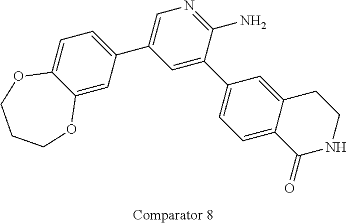 [SMILES: Nc1ncc(-c2ccc3c(c2)OCCCO3)cc1-c1ccc2c(c1)CCNC2=O] | 485 |
| 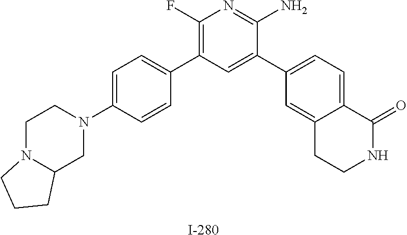 [SMILES: Nc1nc(F)c(-c2ccc(N3CCN4CCCC4C3)cc2)cc1-c1ccc2c(c1)CCNC2=O ; "I-280"] | 1480 | 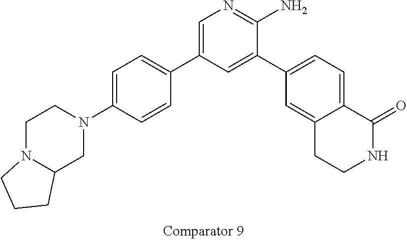 [SMILES: Nc1ncc(-c2ccc(N3CCN4CCCC4C3)cc2)cc1-c1ccc2c(c1)CCNC2=O] | 20 |
| 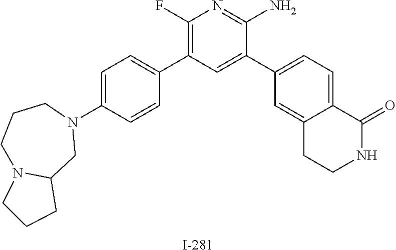 [SMILES: Nc1nc(F)c(-c2ccc(N3CCCN4CCCC4C3)cc2)cc1-c1ccc2c(c1)CCNC2=O ; "I-281"] | 1073 | 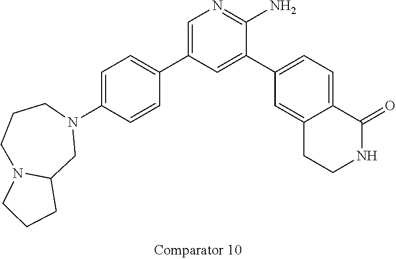 [SMILES: Nc1ncc(-c2ccc(N3CCCN4CCCC4C3)cc2)cc1-c1ccc2c(c1)CCNC2=O] | 92 |
| 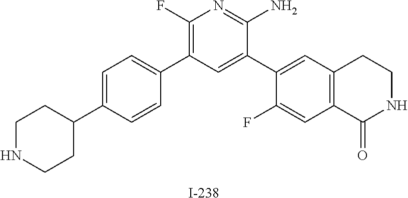 [SMILES: Nc1nc(F)c(-c2ccc(C3CCNCC3)cc2)cc1-c1cc2c(cc1F)C(=O)NCC2 ; "I-238"] | 704 | 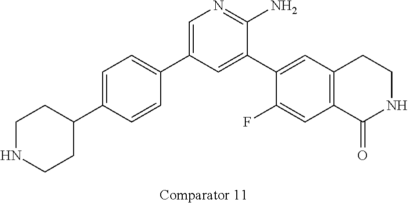 [SMILES: Nc1ncc(-c2ccc(C3CCNCC3)cc2)cc1-c1cc2c(cc1F)C(=O)NCC2] | 27 |

1. A compound of Formula (I) or an enantiomer thereof, or a pharmaceutically acceptable salt, solvate and/or prodrug thereof, 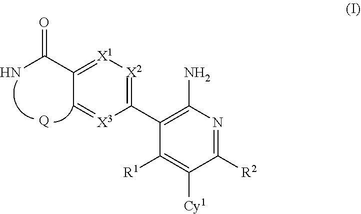
wherein:
X¹ is selected from N and CR³;
X² and X³ are independently selected from N and CR⁴;
Q is C₁₋₄alkylene optionally interrupted by a heteromoiety selected from O, S, S(O), SO₂, and NR⁵ and/or optionally substituted with one or more of R⁶ and/or optionally disubstituted on one carbon with R^{6a} and R^{6b}; provided that when Q comprises the heteromoiety the heteromoiety is separated from the ring amide NH by other than methylene; or
Q is C₂₋₄alkenylene optionally substituted with one or more of R^{6c}; or
Q is selected from C═N and N═C, and is optionally substituted with R^{6c};
R¹ and R² are independently selected from H and halo, provided at least one of R¹ and R² is halo;
R³ is selected from H, halo, OR^{5a}, NR⁷R⁸, C₁₋₆alkyleneNR⁷R⁸ and C₁₋₆alkyl;
each R⁴ is independently selected from H, halo, and C₁₋₆alkyl;
R⁵ and R^{5a} are independently selected from H and C₁₋₆alkyl;
each R⁶ is independently selected from ═O, halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR^{7a}R^{8a}, and C₁₋₆alkyleneNR^{7a}R^{8a};
R^{6a} and R^{6b} are joined to form, together with the carbon atom therebetween, a 3- to 6-, saturated or unsaturated ring optionally containing one heteromoiety selected from N, NH, NC₁₋₆alkyl, O, S, S(O), and SO₂ and optionally substituted with one or more of halo and C₁₋₆alkyl;
each R^{6c} is independently selected from halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl NR^{7a}R^{8a}, and C₁₋₆alkyleneNR^{7a}R^{8a};
R⁷, R^{7a}, R⁸, and R^{8a} are each independently selected from H and C₁₋₆alkyl; or
R⁷ and R⁸ or R^{7a} and R^{8a} are joined to form, together with the nitrogen atom therebetween, a 3- to 7-membered saturated or unsaturated ring, optionally containing one additional heteromoiety selected from N, NH, NC₁₋₆alkyl, O, S, S(O), and SO₂ and optionally substituted with one or more of halo and C₁₋₆alkyl;
Cy¹ is C₆₋₂₀aryl or C₅₋₂₀heteroaryl, and Cy¹ is unsubstituted or is substituted with one or more of R⁹, or
Cy¹ is substituted with Z-Cy², or
Cy¹ is substituted with Z-Cy² and one or more of R⁹;
each R⁹ is independently selected from halo, ═O, CN, NO₂, C₁₋₆alkyl, C₂₋₆alkenyl, C₂₋₆alkynyl, OR¹⁰, C(O)R¹⁰, CO₂R¹⁰, P(O)R¹⁰R¹¹, P(O)(OR¹⁰)(OR¹¹), SR¹⁰, S(O)R¹⁰, SO₂R¹⁰, S(O)(═NR¹¹)R¹⁰, SO₂NR¹⁰R¹¹, SiR¹²R^{12a}R^{12b}, C₁₋₆alkyleneOR¹⁰, OC₁₋₆alkyleneOR¹⁰, C₁₋₆alkyleneNR^{10a}R^{11a}, OC₁₋₆alkyleneNR¹⁰R¹¹, NR¹¹C₁₋₆alkyleneNR¹⁰R¹¹, NR¹¹C₁₋₆alkyleneOR¹⁰, NR^{10a}R^{11a}, NR¹¹COR¹⁰, NR¹¹CO₂R¹⁰, NR¹¹SO₂R¹⁰, C₃₋₇cycloalkyl, C₃₋₇heterocycloalkyl, C₁₋₆alkyleneC₃₋₇cycloalkyl and C₁₋₆alkyleneC₃₋₇heterocycloalkyl, the latter four groups being optionally substituted with one or more of R¹³;
R¹⁰ is selected from H, C₁₋₆alkyl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneOR¹⁴, and C₁₋₆alkyleneNR^{14a}R^{14b}, and all alkyl, alkenyl, alkynyl, alkylene, heterocycloalkyl and cycloalkyl groups of R¹⁰ are optionally substituted with one or more of R¹⁵;
R¹¹ is selected from H and C₁₋₆alkyl; or
R¹⁰ and R¹¹ are joined to form, together with the atom(s) therebetween, a 4- to 6-membered saturated or unsaturated ring, optionally containing one additional heteromoiety selected from N, NR¹⁶, O, S, S(O), and SO₂, and optionally substituted with one or more of R¹⁵;
R^{10a} is selected from H, C₁₋₆alkyl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneOR¹⁴, and C₁₋₆alkyleneNR¹¹R¹⁴, and all alkyl, alkenyl, alkynyl, alkylene, heterocycloalkyl and cycloalkyl groups of R^{10a} are optionally substituted with one or more of R¹⁵;
R^{11a} is selected from H and C₁₋₆alkyl;
R¹², R^{12a} and R^{12b} are independently selected from OR¹⁷, C₁₋₆alkyl, C₃₋₁₁cycloalkyl, C₃₋₁₀heterocycloalkyl, C₁₋₆alkyleneC₃₋₁₀cycloalkyl, and C₁₋₆alkyleneC₃₋₁₀heterocycloalkyl;
each R¹³ is independently selected from halo, OH, C₁₋₆alkyl, OC₁₋₆alkyl, CN and NR^{13a}R^{13b};
R^{13a} and R^{13b} are each independently selected from H and C₁₋₆alkyl;
R¹⁴, R^{14a} and R^{14b} is selected from H and C₁₋₆alkyl;
each R¹⁵ is independently selected from halo, C₁₋₆alkyl, CN and NR^{15a}R^{15b};
R^{15a} and R^{15b} are each independently selected from H and C₁₋₆alkyl;
R¹⁶ is selected from H and C₁₋₆alkyl;
R¹⁷ is selected from H, C₁₋₆alkyl, C₃₋₁₁cycloalkyl, C₃₋₁₀heterocycloalkyl, C₁₋₆alkyleneC₃₋₁₀cycloalkyl, C₁₋₆alkyleneC₃₋₁₀heterocycloalkyl C₁₋₆alkyleneOR¹⁸, and C₁₋₆alkyleneNR¹⁸R¹⁹;
R¹⁸ and R¹⁹ are independently selected from H and C₁₋₆alkyl;
Z is absent, or is selected from C₁₋₆alkylene, O, C(O), CO₂, S, S(O), SO₂, S(O)(═NR^{11b}), NR^{11b}, C₁₋₆alkyleneO, C₁₋₆alkyleneC(O), C₁₋₆alkyleneCO₂, C₁₋₆alkyleneS, C₁₋₆alkyleneS(O), C₁₋₆alkyleneSO₂, C₁₋₆alkyleneS(O)(═NR^{11b}) C₁₋₆alkyleneNR^{11b} OC₁₋₆alkylene, C(O)C₁₋₆alkylene, CO₂C₁₋₆alkylene, SC₁₋₆alkylene, S(O)C₁₋₆alkylene, SO₂C₁₋₆alkylene, S(O)(═NR^{11b})C₁₋₆alkylene and NR^{11b}C₁₋₆alkylene;
R^{11b} is selected from H and C₁₋₆alkyl;
Cy² is selected from C₃₋₁₄cycloalkyl and C₃₋₁₄heterocycloalkyl, and Cy² is unsubstituted or substituted with one or more of R²⁰;
each R²⁰ is independently selected from halo, ═O, CN, OH, C₁₋₆alkyl, C₂₋₆alkenyl, C₂₋₆alkynyl, C₃₋₁₀cycloalkyl, C₃₋₁₀heterocycloalkyl, C₆₋₁₁aryl, C₅₋₁₄heteroaryl, C₁₋₆alkyleneC₃₋₁₀cycloalkyl, C₁₋₆alkyleneC₃₋₁₀heterocycloalkyl, C₁₋₆alkyleneC₆₋₁₁aryl, C₁₋₆alkyleneC₅₋₁₄heteroaryl, OC₁₋₆alkyl, OC₂₋₆alkenyl, OC₂₋₆alkynyl, C₁₋₆alkyleneOR²¹, OC₁₋₆alkyleneOR²¹, C₁₋₆alkyleneNR²²R²³, OC₁₋₆alkyleneNR²²R²³, SC₁₋₆alkyl, SC₂₋₆alkenyl, SC₂₋₆alkynyl, C(O)C₁₋₆alkyl, C(O)C₂₋₆alkenyl, C(O)C₂₋₆alkynyl, C(O)C₃₋₁₀cycloalkyl, C(O)C₃₋₁₀heterocycloalkyl, C(O)C₆₋₁₁aryl, C(O)C₅₋₁₄heteroaryl, C(O)C₁₋₆alkyleneC₃₋₁₀cycloalkyl, C(O)C₁₋₆alkyleneC₃₋₁₀heterocycloalkyl, C(O)C₁₋₆alkyleneC₆₋₁₁aryl, C(O)C₁₋₆alkyleneC₅₋₁₄heteroaryl, C(O)C₁₋₆alkylenylOR²¹, C(O)C₁₋₆alkyleneNR²²R²³, C(O)C₁₋₆alkyleneOC₁₋₆alkyleneNR²²R²³, C(O)NR²²R²³, CO₂C₁₋₆alkyl, CO₂C₂₋₆alkenyl, CO₂C₂₋₆alkynyl, CO₂C₁₋₆alkyleneOR²¹, CO₂C₁₋₆alkyleneOC₁₋₆alkyleneNR²²R²³, NR²²R²³, NR²⁴C₃₋₁₀cycloalkyl, NR²⁴C₃₋₁₀heterocycloalkyl, NR²⁴C₆₋₁₁aryl, NR²⁴C₅₋₁₄heteroaryl, NR²⁴C₁₋₆alkyleneOR²¹, NR²⁴C₁₋₆alkyleneNR²²R²³, NR²⁴C₁₋₆alkyleneC₃₋₁₀cycloalkyl, NR²⁴C₁₋₆alkyleneC₃₋₁₀heterocycloalkyl, NR²⁴C₁₋₆alkyleneC₆₋₁₁aryl, NR²⁴C₁₋₆alkyleneC₅₋₁₄heteroaryl, NR²⁴SO₂C₁₋₆alkyl, SO₂C₁₋₆alkyl, SO₂C₂₋₆alkenyl, SO₂C₂₋₆alkynyl, and SO₂NR²²R²³, and alkyl, alkenyl, alkynyl, alkylene, aryl, heteroaryl, heterocycloalkyl and cycloalkyl groups of R²⁰ are optionally substituted with one or more of R²⁵;
R²¹ is selected from H, C₁₋₆alkyl, C₁₋₆alkyleneOC₁₋₆alkyl, C₃₋₁₁cycloalkyl, C₃₋₁₀heterocycloalkyl, C₆₋₁₁aryl, C₅₋₁₄heteroaryl C₂₋₆alkenyl, C₂₋₆alkynyl, C₁₋₆alkyleneC₃₋₁₀cycloalkyl, and C₁₋₆alkyleneC₃₋₁₀heterocycloalkyl, C₁₋₆alkyleneC₆₋₁₁aryl and C₁₋₆alkyleneC₅₋₁₄heteroaryl;
R²² is selected from H and C₁₋₆alkyl;
R²³ is selected from H, C₁₋₆alkyl, C₁₋₆alkyleneOC₁₋₆alkyl, C₃₋₁₀cycloalkyl, C₃₋₁₀heterocycloalkyl, C₁₋₆alkyleneC₃₋₁₀cycloalkyl, C₆₋₁₁aryl, C₅₋₁₄heteroaryl C₁₋₆alkyleneC₃₋₁₀heterocycloalkyl, C₁₋₆alkyleneC₆₋₁₁aryl and C₁₋₆alkyleneC₅₋₁₄heteroaryl, or
R²² and R²³ are joined to form, together with the nitrogen atom therebetween, a 4- to 6-membered saturated or unsaturated ring, optionally containing one additional heteromoiety selected from N, NR²⁶, O, S, S(O), and SO₂, and optionally substituted with one or more of halo and C₁₋₆alkyl;
R²⁴ is selected from H and C₁₋₆alkyl;
each R²⁵ is selected from halo, C₁₋₆alkyl, CN and NR^{25a}R^{25b};
R^{25a} and R^{25b} are each independently selected from H and C₁₋₆alkyl; and
R²⁶ is selected from H and C₁₋₆alkyl;
wherein all available hydrogen atoms are optionally substituted with a fluorine atom.

2. The compound of claim 1, wherein X¹ is N, or wherein X¹ is CR³ and R³ is selected from H, F, Cl, OR^{5a}, NR⁷R⁸, C₁₋₄alkyleneNR^{7a}R^{8a} and C₁₋₄alkyl and all available hydrogen atoms are optionally substituted with a fluorine atom.

3. (canceled)

4. (canceled)

5. The compound of claim 1, wherein Q is C₁₋₃alkylene and optionally substituted with one to three of R⁶, each R⁶ is independently selected from ═O, F, Cl, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC₁₋₄alkyl, NR^{7a}R^{8a}, and C₁₋₄alkyleneNR^{7a}R₈ₐ, or
Q is C═C optionally substituted with one or two of R^{6c} independently selected from F, Cl, C₁₋₄alkyl and OC₁₋₄alkyl; or
Q is selected from C═N and N═C and is optionally substituted with R^{6c} , independently selected from F, Cl, OH, OC₁₋₄alkyl and NR^{7a}R^{8a}; or
Q is C₁₋₃alkylene optionally interrupted by NR⁵, or
Q is C₁₋₃alkylene and is disubstituted on one carbon with R^{6a} and R^{6b},
R⁵ is selected from H and C₁₋₄alkyl;
R^{6a} and R^{6b} are joined to form, together with the carbon atom therebetween, a 3- to 6-membered saturated or unsaturated ring optionally containing one heteromoiety selected from N, NH, NC₁₋₆alkyl, O, S, S(O), and SO₂ and optionally substituted with one to four of halo and C₁₋₄alkyl;
R^{7a} and R^{8a} are each independently selected from H and C₁₋₄alkyl; and
all available hydrogen atoms are optionally substituted with a fluorine atom.

6.-18. (canceled)

19. The compound of claim 1 wherein one of X² and X³ is N and the other is CR⁴, or X² and X³ are both CR⁴ or both N, each R⁴ is independently selected from H and F.

20.-25. (canceled)

26. The compound of claim 1, wherein R¹ is H and R² is F.

27. The compound of claim 1, wherein Cy¹ is phenyl, and Cy¹ is unsubstituted, or is substituted with one to three of R⁹, or is substituted with Z-Cy², or is substituted with Z-Cy² and one to three of R⁹; or
Cy¹ is a bicyclic C₉₋₁₁aryl wherein the aryl is fused to a heterocycloalkyl, and Cy¹ is unsubstituted, or is substituted with one to three of R⁹, or is substituted with Z-Cy², or is substituted with Z-Cy² and one to three of R⁹; or
Cy¹ is a tricyclic C₁₁₋₁₇aryl wherein the aryl group is fused and/or spiro fused to one or two heterocycloalkyl groups, and Cy¹ is unsubstituted, or is substituted with one to three of R⁹, or is substituted with Z-Cy², or is substituted with Z-Cy² and one to three of R⁹, or
Cy¹ is C₅₋₆heteroaryl, and Cy¹ is unsubstituted, or is substituted with one to three of R⁹, or is substituted with Z-Cy², or is substituted with Z-Cy² and one or more of R⁹, or
Cy¹ is bicyclic C₉₋₁₁heteroaryl, and Cy¹ is unsubstituted, or is substituted with one to three of R⁹, or is substituted with Z-Cy², or is substituted with Z-Cy² and one or more of R⁹.

28.-40. (canceled)

41. The compound of claim 27, wherein each R⁹ is independently selected from halo, ═O, CN, NO₂, C₁₋₆alkyl, C₂₋₆alkenyl, C₂₋₆alkynyl, OR¹⁰, C(O)R¹⁰, CO₂R¹⁰, P(O)R¹⁰R¹¹, P(O)(OR¹⁰)(OR¹¹), SR¹⁰, S(O)R¹⁰, SO₂R¹⁰, S(O)(═NR¹¹)R¹⁰, SO₂NR¹⁰R¹¹, SiR¹²R^{12a}R^{12a}, C₁₋₆alkyleneOR¹⁰, OC₁₋₆alkyleneOR¹⁰, C₁₋₆alkyleneNR^{10a}R^{11a}, OC₁₋₆alkyleneNR¹⁰R¹¹, NR¹¹C₁₋₆alkyleneNR¹⁰R¹¹, NR¹¹C₁₋₆alkyleneOR¹⁰, NR^{10a}R^{11a} NR¹¹COR¹⁰, NR¹¹CO₂R¹⁰, NR¹¹SO₂R¹⁰, C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to three of R¹³ and all available hydrogen atoms are optionally substituted with a fluorine atom.

42. The compound of claim 41, wherein one to three R⁹ are selected from F, Cl, CN, CH₃, CH₂CH₃, CH(CH₃)₂, CH₂CH₂CH₃, CH₂CH₂CH₂CH₃, CF₂H, CF₃, CFH₂, CH₂CF₂H, CH₂CF₂H, CH₂CH₂F₂H, CH₂CH₂CH₂F₂H and CH(CH₃)₃.

43. (canceled)

44. (canceled)

45. The compound of claim 41, wherein each R⁹ is independently selected from F, Cl, CN, NO₂, C₁₋₄alkyl, OR¹⁰, C(O)R¹⁰, CO₂R¹⁰, P(O)R¹⁰R¹¹, P(O)(OR¹⁰)(OR¹¹), SR¹⁰, S(O)R¹⁰, SO₂R¹⁰, S(O)(═NR¹¹)R¹⁰, SO₂NR¹⁰R11, SiR¹²R^{12a}R^{12b}, C₁₋₄alkyleneOR¹⁰, OC₁₋₄alkyleneOR¹⁰, C₁₋₄alkyleneNR^{10a}R^{11a}, OC₁₋₄alkyleneNR¹⁰R¹¹, NR¹¹C₁₋₄alkyleneNR¹⁰R¹¹, NR¹¹C₁₋₄alkyleneOR¹⁰, NR^{10a}R^{11a} NR¹¹COR¹⁰, NR¹¹CO₂R¹⁰, NR¹¹SO₂R¹⁰, C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to three of R¹³ and all available hydrogen atoms groups are optionally substituted with a fluorine atom.

46. (canceled)

47. (canceled)

48. The compound of claim 45, wherein one or two R⁹ are selected from C₁₋₄alkyleneNR^{10a}R^{11a} NR^{10a}R^{11a} and NR¹¹C₁₋₆alkyleneNR¹⁰R¹¹,
wherein R^{10a} is selected from H and C₁₋₄alkyl wherein C₁₋₄alkyl is optionally substituted with one to three of R¹⁵, and R^{11a} is selected from H and C₁₋₄alkyl; or
wherein R^{10a} is selected from C₁₋₆alkyleneOR¹⁴ and C₁₋₆alkyleneNR^{14a}R^{14b} and all alkylene groups in R^{10a} are optionally substituted with one to three of R¹⁵, each R¹⁵ is independently selected from F, Cl, CN, C₁₋₄alkyl and NR^{15a}R^{15b} and R^{15a} and R^{15b} are independently selected from H and C₁₋₄alkyl; or
wherein R^{10a} is C₁₋₆alkyleneC₃₋₇heterocycloalkyl and R^{11a} is selected from H and C₁₋₄alkyl;
and all available hydrogen atoms are optionally substituted with a fluorine atom.

49.-57. (canceled)

58. The compound of claim 45, wherein one or two of R⁹ are selected from C₃₋₇cycloalkyl, C₃₋₇heterocycloalkyl, C₁₋₄alkyleneC₃₋₇cycloalkyl and C₁₋₄alkyleneC₃₋₇heterocycloalkyl, each of which is optionally substituted with one to four of R¹³; and
each R¹³ is independently selected from F, Cl, CN, OH, C₁₋₄alkyl, OC₁₋₄alkyl and NR^{13a}R^{13b} and R^{13a} and R^{13b} are each independently selected from H and C₁₋₄alkyl;
and all available hydrogen atoms are optionally substituted with a fluorine atom.

59.-97. (canceled)

98. The compound of claim 1, wherein Z is absent or wherein Z is selected from C₁₋₄alkylene, O, C(O), CO₂, SO₂, S(O)(═NR^{11b}), and NR^{11b}, and R^{11b} is selected from H and C₁₋₄alkyl, and all available hydrogen atoms are optionally substituted with a fluorine atom.

99.-102. (canceled)

103. The compound of claim 1, wherein Z is selected from C₁₋₄alkyleneO, C₁₋₄alkyleneC(O), C₁₋₆alkyleneCO₂, C₁₋₄alkyleneS, C₁₋₄alkyleneS(O), C₁₋₄alkyleneSO₂, C₁₋₄alkyleneNR^{11b}, OC₁₋₄alkylene, C(O)C₁₋₄alkylene, CO₂C₁₋₄alkylene, SC₁₋₄alkylene, S(O)C₁₋₄alkylene, SO₂C₁₋₄alkylene, and NR^{11b}C₁₋₆alkylene, and R^{11b} is selected from H and C₁₋₄alkyl, and all available hydrogen atoms are optionally substituted with a fluorine atom.

104. (canceled)

105. (canceled)

106. The compound of claim 1, wherein Cy² is C₃₋₁₁cycloalkyl and Cy² is unsubstituted or substituted with one to three of R²⁰, or
wherein Cy² is a monocyclic C₃₋₇heterocycloalkyl and Cy² is unsubstituted or substituted with one to three of R²⁰, or
wherein Cy² is a bicyclic heterocycle and Cy² is unsubstituted or substituted with one to four of R²⁰.

107.-120. (canceled)

121. The compound of claim 106, wherein each R²⁰ is independently selected from F, Cl, ═O, CN, OH, C₁₋₄alkyl, C₂₋₄alkenyl, C₂₋₄alkynyl, C₃₋₁₀cycloalkyl, C₃₋₁₀heterocycloalkyl, C₆₋₁₁aryl, C₅₋₁₄heteroaryl, C₁₋₄alkyleneC₃₋₁₀cycloalkyl, C₁₋₄alkyleneC₃₋₁₀heterocycloalkyl, C₁₋₄alkyleneC₆₋₁₁aryl, C₁₋₄alkyleneC₅₋₁₄heteroaryl, OC₁₋₄alkyl, OC₂₋₄alkenyl, OC₂₋₄alkynyl, C₁₋₄alkyleneOR²¹, OC₁₋₄alkyleneOR²¹, C₁₋₄alkyleneNR²²R²³, OC₁₋₄alkyleneNR²²R²³, SC₁₋₄alkyl, SC₂₋₄alkenyl, SC₂₋₄alkynyl, C(O)C₁₋₄alkyl, C(O)C₂₋₄alkenyl, C(O)C₂₋₄alkynyl, C(O)C₃₋₁₀cycloalkyl, C(O)C₃₋₁₀heterocycloalkyl, C(O)C₆₋₁₁aryl, C(O)C₅₋₁₄heteroaryl, C(O)C₁₋₄alkyleneC₃₋₁₀cycloalkyl, C(O)C₁₋₄alkyleneC₃₋₁₀heterocycloalkyl, C(O)C₁₋₄alkyleneC₆₋₁₁aryl, C(O)C₁₋₄alkyleneC₅₋₁₄heteroaryl, C(O)C₁₋₄alkylenylOR²¹, C(O)C₁₋₄alkyleneNR²²R²³, C(O)C₁₋₄alkyleneOC₁₋₄alkyleneNR²²R²³, C(O)NR²²R²³, CO₂C₁₋₆alkyl, CO₂C₂₋₄alkenyl, CO₂C₂₋₄alkynyl, CO₂C₁₋₄alkyleneOR²¹, CO₂C₁₋₄alkyleneOC₁₋₄alkyleneNR²²R²³, NR²²R²³, NR²⁴C₃₋₁₀cycloalkyl, NR²⁴C₃₋₁₀heterocycloalkyl, NR²⁴C₆₋₁₁aryl, NR²⁴C₅₋₁₄heteroaryl NR²⁴C₁₋₄alkyleneC₃₋₁₀cycloalkyl, NR²⁴C₁₋₄alkyleneC₃₋₁₀heterocycloalkyl, NR²⁴C₁₋₄alkyleneC₆₋₁₁aryl, NR²⁴C₁₋₄alkyleneC₅₋₁₄heteroaryl NR²⁴C₁₋₄alkyleneOR²¹, NR²⁴SO₂C₁₋₄alkyl, SO₂C₁₋₆alkyl, SO₂C₂₋₄alkenyl, SO₂C₂₋₄alkynyl, and SO₂NR²²R²³, and alkyl, alkenyl, alkynyl, alkylene, aryl, heteroaryl, heterocycloalkyl, and cycloalkyl groups of R²⁰ are optionally substituted with one to four of R²⁵,
wherein R²² is selected from H and C₁₋₄alkyl, and
R²³ is selected from H, C₁₋₄alkyl, C₁₋₄alkyleneOC₁₋₄alkyl, C₃₋₁₀cycloalkyl, C₃₋₁₀heterocycloalkyl, C₆₋₁₁aryl, C₅₋₁₄heteroaryl, C₁₋₄alkyleneC₃₋₁₀cycloalkyl, C₁₋₄alkyleneC₃₋₁₀heterocycloalkyl, C₁₋₆alkyleneC₆₋₁₁aryl and C₁₋₆alkyleneC₅₋₁₄heteroaryl; or
R²² and R²³ are joined to form, together with the nitrogen atom therebetween, a 4- to 6-membered saturated or unsaturated ring, optionally containing one additional heteromoiety selected from N, NR²⁶, O, S, S(O), and SO₂ and optionally substituted with one to three of halo and C₁₋₆alkyl, and
each R²⁵ is independently selected from F, Cl, CN, C₁₋₄alkyl and NR^{25a}R^{25b}, and R^{25a} and R^{25b} are independently selected from H and C₁₋₄alkyl, and
R²⁶ is selected from H and C₁₋₄alkyl;
and all available hydrogen atoms are optionally substituted with a fluorine atom.

122.-129. (canceled)

130. The compound of claim 121, wherein each R²⁰ is independently selected from F, Cl, ═O, CN, OH, CH₃, CH₂CH₃, CH(CH₃)₂, C(CH₃)₃, CF₂H, CF₃, CFH₂, CH₂CFH₂, CH₂CF₂H, CH₂CF₃, CH₂CH₂CF₂H, CH₂CH₂CH₂CF₂H, CH₂CH₂CF₃, CH₂CH₂CH₂CF₃, C₃₋₆cycloalkyl, C₃₋₆heterocycloalkyl, phenyl, C₅₋₁₄heteroaryl, C₁₋₄alkyleneC₃₋₆cycloalkyl, C₁₋₄alkyleneC₃₋₆heterocycloalkyl, C₁₋₄alkylenephenyl, C₁₋₄alkyleneC₅₋₁₄heteroaryl, OC₁₋₄alkyl, C₁₋₄alkyleneOH, C₁₋₄alkyleneOCH₃, C(O)C₁₋₄alkyl, C(O)C₃₋₆cycloalkyl, C(O)C₃₋₆heterocycloalkyl, C(O)C₁₋₂alkyleneC₃₋₆cycloalkyl, C(O)C₁₋₂alkyleneC₃₋₁₀heterocycloalkyl, C(O)C₁₋₄alkyleneOCH₃, C(O)C₁₋₄alkyleneOH, C(O)C₁₋₄alkyleneNH₂, C(O)C₁₋₄alkyleneN(CH₃)₂, C(O)C₁₋₄alkyleneOC₁₋₄alkyleneN(CH₃)₂, CO₂C₁₋₆alkyl, CO₂C₁₋₄alkyleneOC₁₋₆alkyl, NHC₁₋₄alkyl, NC₁₋₄alkylC₁₋₄alkyl, NHC₁₋₂alkyleneC₃₋₁₀cycloalkyl, NCH₃C₁₋₂alkyleneC₃₋₁₀cycloalkyl, NCH₃C₁₋₂alkyleneC₃₋₁₀heterocycloalkyl, NHC₁₋₂alkyleneC₃₋₁₀heterocycloalkyl, NR²⁴C₁₋₄alkyleneOH, NR²⁴C₁₋₄alkyleneOCH₃, NHSO₂C₁₋₄alkyl, NCH₃SO₂C₁₋₄alkyl, and SO₂C₁₋₆alkyl, and alkyl, alkylene, aryl, heteroaryl, heterocycloalkyl and cycloalkyl groups of R²⁰ are optionally substituted with one to three of R²⁵, and R²⁵ is independently selected from one or more of F, Cl, and C₁₋₄alkyl, and all available hydrogen atoms are optionally substituted with a fluorine atom.

131. (canceled)

132. The compound of claim 1, wherein the compound of Formula (I) is selected from the compounds listed in Table 1,
or a pharmaceutically acceptable salt, solvate and/or prodrug thereof.

133.-135. (canceled)

136. A method of treating cancer comprising administering a therapeutically effective amount of one or more compounds of claim 1, and/or a pharmaceutically acceptable salt, solvate and/or prodrug thereof, to a subject in need thereof.

137. (canceled)

138. A method of inhibiting proliferative activity in a cell, comprising administering an effective amount of one or more compounds of claim 1, and/or a pharmaceutically acceptable salt, solvate and/or prodrug thereof, to the cell.

139. (canceled)

140. (canceled)

141. A pharmaceutical composition comprising one or more compounds of claim 1, and/or a pharmaceutically acceptable salt, and/or solvate thereof, and a pharmaceutically acceptable carrier and/or diluent.

142. (canceled)
